(19) United States
(12) Patent Application Publication
(10) Pub. No.: US 2025/0154109 A1
(43) Pub. Date: May 15, 2025

(54) SODIUM CHANNEL BLOCKING COMPOUNDS, DERIVATIVES THEREOF, AND METHODS OF THEIR USE

(71) Applicant: LATIGO BIOTHERAPEUTICS, INC, Thousand Oak, CA (US)

(72) Inventors: Ashok Bajji, Salt Lake City, UT (US); Michael D. Kaufman, Thousand Oaks, CA (US); Bryan Moyer, Thousand Oaks, CA (US); James Michael Balkovec, Martinsville, NJ (US); Andrew Stewart Tasker, Simi Valley, CA (US)

(21) Appl. No.: 18/833,138

(22) PCT Filed: Feb. 2, 2023
(86) PCT No.: PCT/US2023/012172

§ 371 Date: Jul. 25, 2024

Related U.S. Application Data
(60) Provisional application No. 63/354,509, filed on Jun. 22, 2022.
(60) Provisional application No. 63/306,695, filed on Feb. 4, 2022.

(51) Int. Cl. C07D 223/06; A61K 31/4545; A61K 31/5375; A61K 31/5377; A61K 31/55; A61K 31/551; C07D 211/34; C07D 211/38; C07D 213/82; C07D 237/24; C07D 265/30; C07D 401/04; C07D 401/12; C07D 401/14; C07D 403/10; C07D 403/14; C07D 405/14; C07D 409/04; C07D 409/14; C07D 413/04; C07D 413/14; C07D 417/14; C07D 491/10

(52) U.S. Cl. CPC C07D 223/06; A61K 31/4545; A61K 31/5375; A61K 31/5377; A61K 31/55; A61K 31/551; C07D 211/34; C07D 211/38; C07D 213/82; C07D 237/24; C07D 265/30; C07D 401/04; C07D 401/12; C07D 401/14; C07D 403/10; C07D 403/14; C07D 405/14; C07D 409/04; C07D 409/14; C07D 413/04; C07D 413/14; C07D 417/14; C07D 491/10

(57) ABSTRACT

The invention provides compounds that are useful for treatment of conditions associated with aberrant activity of voltage gated sodium channel Nav1.8, and methods of treating a subject with those compounds for conditions such as pain, itch, and cough.

### FIELD OF THE INVENTION

The application relates generally to sodium channel blocking compounds, derivatives thereof, and the use of such compounds as pharmacological agents.

### BACKGROUND

Millions of people suffer from conditions associated with pain, itch, and/or cough. Pain can be a symptom or cause of conditions such as neuropathy, hyperalgesia, and opioid use disorders. In many cases, drugs used to treat such condition fail to provide relief or produce intolerable side effects. Therefore, existing treatments are inadequate for many patients who suffer from a variety of conditions.

### SUMMARY

The invention provides compounds that are useful for treatment of conditions associated with aberrant activity of voltage gated sodium channel Nav1.8, such as pain, itch, and cough.

In one aspect, the invention provides a compound of Formula (I):

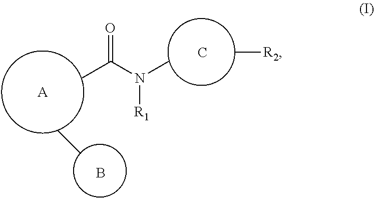

wherein:
   A is an aryl or heteroaryl ring containing one or more heteroatoms independently selected from O, S, and N, wherein A is unsubstituted or substituted with one or more substituents selected from a group consisting of:
      H, halo, C₁-C₆ alkyl, C₁-C₆ branched alkyl, C₁-C₆ alkenyl, C₁-C₆ alkynyl, C₁-C₆ haloalkyl, C₁-C₆ alkoxy, C₁-C₆ cycloalkoxy, C₁-C₆ haloalkoxy, nitro, cyano, —CH₂—(C₁-C₆)-cycloalkyl, —CF₂-cycloalkyl, —CH(CH₃)-cycloalkyl, —CH₂-aryl, —CF₂-aryl, —CH(—CH₃)-aryl, C(═O)—(C₁-C₆)-alkyl, —C(═O)-cycloalkyl, —C(═O)—NH-alkyl, —C(═O)NH₂, hydroxy, —COOH (and ester thereof), alkylsulfonyl, arylsulfonyl, sulfonamide, amino, —NR′R″, —NHSO₂R₁, —NHC(═O)-alkyl —NH(C═O)NR′R″, trifluoromethyl, bromo, chloro, fluoro, iodo, cyclopropylmethyl, sulfonylmethyl; 3-6-membered cycloalkyl; 3-6-membered heterocycloalkyl, 5-7 membered aryl, or 5-7 membered heteroaryl, any of which may have one or more substituents listed herein, wherein the 3-6 membered heterocycloalkyl or 5-7 membered heteroaryls comprise at least one heteroatom independently selected from O, S, and N;
   B is selected from the group consisting of a monocyclic, bicyclic, or spirocyclic cycloalkyl ring, and a monocyclic, bicyclic, or spirocyclic cycloheteroalkyl ring, wherein the cycloheteroalkyl contains 1-4 heteroatoms independently selected from N, O, and S, and wherein one or more carbons of the cycloalkyl or cycloheteroalkyl ring may be optionally substituted with one or more halogens or one or more haloalkyl groups; C is selected from the group consisting of aryl, heteroaryl, heterocyclyl, cycloalkyl, and bridged cycloalkyl, wherein said aryl, heteroaryl, heterocyclyl, and bridged cycloalkyl comprises one or more substitutions (in addition to the R₂ group) selected from a group consisting of H, halo, alkyl, cyano, haloalkyl, and nitro;
   R₁ is selected from H and C₁-C₃ alkyl;
   R₂ is Formula (II):

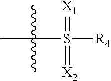

wherein:
X₁ is O and X₂ is NH or NR′;
X₁ is O and X₂ is NR₃; or
X₁ and X₂ are independently selected from NH or NR′;
wherein,
R₃ is independently selected from —(C═O)—(CH₂)ₙRₐ, —(C═O)—(CH₂)ₙ—(OCH₂CH₂)ₙRₐ, —(C═O)—(CH₂)ₙ—(OCH₂CH₂O)ₙRₐ, —(C═O)—(CH₂)ₙ—(NHCH₂CH₂)ₙRₐ, —(C═O)—(CH₂)ₙ—(NHCH₂CH₂O)ₙRₐ, —(CH₂)ₙRₐ, —(CH₂)ₙ(CR_{b}R_{c})Rₐ, —(C═O)—(NHCH₂CH₂NH)ₙRₐ, —(C═O)—(CH R_{b})Rₐ, —(CH₂)ₙ(CR_{b}R_{c}) (CH₂)ₙRₐ, or —(C═O)—(CR_{b}R_{c})Rₐ;
Rₐ is H, C₁-C₆-alkyl, branched alkyl, cycloakyl, aryl, heteroaryl, alkenyl, alkynyl, haloalkyl, alkoxy, cycloalkoxy, haloalkoxy, —CF₂-cycloalky, —CH(CH₃)-cycloalkyl, —CF₂-aryl, —NH₂, —NR′R″, amino cycloalkyl, 4 to 7 member heterocyclyl, —OH, —COONH₂, —COOH (and ester thereof), alkylsulfonyl, arylsulfonyl, sulfonamide, or amino;
R_{b} is selected from F, C₁-C₆-alkyl, haloalkyl, branched alkyl, aryl, heteroaryl, 3-7 membered carbocylyl, 4-7 membered heterocyclyl with one or more heteroatoms;
R_{c} is C₁-C₆-alkyl or F;
R_{c} R_{b} together with the carbon atom to which attached form a 3-6 membered carbocyclic ring or 4-6 membered heterocyclic ring with one or more hetero atoms;
R_{b} is selected from F, C₁-C₆-alkyl, haloalkyl, branched alkyl, aryl, heteroaryl, 3-7 membered carbocylyl, 4-7 membered heterocyclyl with one or more heteroatoms;
R_{c} is C₁-C₆-alkyl or F;
R_{c} R_{b} together with the carbon atom to which attached form a 3-6 membered carbocyclic ring or 4-6 member heterocyclic ring with one or more hetero atoms;
R₄ is selected from C₁-C₃ alkyl, C₃-C₄ cycloalkyl, haloalkyl, halocycloalkyl, aryl, heteroaryl, or heterocyclyl; and
n=0, 1, 2, 3, 4, 5, 6.

In certain embodiments, R₁ is selected from H and C₁-C₃ alkyl

In certain embodiments, R₁ is H.

In certain embodiments, R₂ is H.

In various embodiments, if the A ring is a heteroaryl, it contains one or more nitrogen atom in a ring. In certain embodiments, the A ring contains one nitrogen atom. In certain embodiments, such nitrogen atom may be in the form of an N-oxide, wherein the N-oxide is selected from a group consisting of pyridyl N-oxide, pyrazinyl N-oxide, and pyrimidinyl N-oxide, pyridazinyl N-oxide. In certain embodiments, the A ring may include further substituents.

In various embodiments, ring C is optionally fused to at least one ring selected from the group consisting of: an optionally saturated carbocyclyl containing 5-6 ring members or an optionally saturated heterocyclyl containing 5-6 ring members and 1-4 optionally charged heteroatoms.

In certain embodiments, R₂ is selected from H or formula (II):

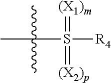

wherein:
m and p are either 0 or 1; and
X₁ is O and X₂ is either NH or NR′; or
X₁ is O and X₂ is NR₃
R₃ is defined above.

In various embodiments, R₂ is selected from a group consisting of: H, —C(═O)NH₂, —C(═O)NHR′, —C(═O)NR′R″, —C(═O)OH, alkylamino, and —S(═O)R′.

In various embodiments, R₂ is selected from H or Formula (II):

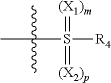

wherein:
m and p are either 0 or 1; and
X₁ is O, NH, NR′ and X₂ is either NH or NR′, and R₄ is described above.

In certain embodiments, R₂ is represented by Formula (II):

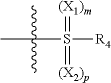

wherein:
m and p are either 0 or 1;
X₁ and X₂ are both O or NH; and R₄ is described above.

In certain embodiments, R₂ is represented by Formula (II) (described above), wherein X₁ is not O and/or X₂ is not O.

In certain embodiments, R₂ is represented by Formula (II) (described above), wherein X₁ is not NH or NR′ and X₂ is not NH or NR′.

In certain embodiments, ring A is an optionally substituted aryl.

In certain embodiments, ring A is an optionally substituted heteroaryl with one heteroatom.

In certain embodiments, ring A is an optionally substituted heteroaryl with two heteroatoms.

In certain embodiments, ring A is substituted with at least trifluoromethyl.

In certain embodiments, trifluoromethyl is the only substitution on ring A.

In certain embodiments, ring A is substituted with at least cyclopropylmethyl.

In certain embodiments, A is represented by the following Formula(s):

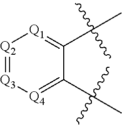

wherein each of Q₁, Q₂, Q₃ and Q₄ is independently N, N—O, or CR₅;
wherein R₅ is H, hydroxyl, halogen, —CD₃, alkyl, haloalkyl, alkoxy, haloalkoxy, alkyl sulfonyl, alkyl sulfoximinyl, alkyl sulfonamide, cyano, —CF₃, —OCF₃, heterocyclyl in which each ring has 4 to 6 members, heteroaryl having a 5 or 6 ring membered, saturated heterocyclyl, or partially unsaturated heterocyclyl, O-aryl in which each ring has 5 or 6 members, O-heteroaryl in which each ring has 5 or 6 members, O-cycloalkyl, O-cycloheteroalkyl, each of which is optionally substituted.

In certain embodiments, ring A is an optionally substituted five (5) or more membered saturated or partially unsaturated heterocyclic ring having one or two heteroatoms independently selected from N, O, or S.

In certain embodiments, ring A is represented by the following Formula(s):

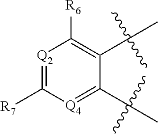

wherein:
Q₂ and Q₄ is N, N—O;
Q₂ is N, N—O; Q₄ is CR₅;
Q₂ is CR₅; Q₄ is N, N—O;
R₆ is H, halogen, —CD₃, C₁-C₆ alkyl, branched alkyl, alkenyl, alkynyl, haloalkyl, alkoxy, cycloalkyl, heterocyclyl, NH₂, NHR′, NR′R″, NHC(═O)R′, NHSO₂R, —C(═O)R′, —C(═O)NHR′, —C(═O)NR′R″, —O—R′, —O—C(═O)—R′, aryl, heteroaryl, —CF₂CH₃, —CF₂CF₃;
R₇ is H, hydroxyl group, halogen, —CD₃, C₁-C₆ alkyl, branched alkyl, allyl, alkenyl, alkynyl, haloalkyl, alkoxy, cycloalkoxy, haloalkoxy, nitro, cyano, —CH₂-cycloalkyl, —CF₂CH₃, —CF₂CF₃, CH₂CF₂, —CF₂-cycloalky, —CH(CH₃)-cycloalkyl, —CH₂-aryl, —CF₂-heteroaryl, —CF₂-heterocyclyl, —CH(—CH₃)-aryl, C(═O)-alkyl, —C(═O)cycloalkyl, —C(═O)—NH-alkyl, —C(═O)NH₂, —C(═O)NHR′, —C(═O)NR′R″, hydroxy, —COOH (and ester thereof), alkylsulfonyl, arylsulfonyl, sulfonamide, amino, NR′R″, —NHSO₂R′, —NHC(═O)-alkyl —NH(C═O)NR′R″, trifluoromethyl, cyclopropylmethyl, methylsulfonyl, 3-6 membered cycloalkyl; 3-6 membered heterocycloalkyl, any of which may have one or more substituents, wherein the 3-6 membered heterocycloalkyl comprises at least one heteroatom independently selected from O, S, and N;
wherein not more than two of Q₁, Q₂, Q₃ and Q₄ are N or N—O.

In certain embodiments, ring A is represented by the following Formula(s):

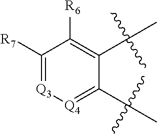

wherein:
Q₃ and Q₄ are N; wherein Q₃ is N, N—O; Q₄ is CR₅; or wherein Q₃ is CR₅, Q₄ is N, N—O; R₅ is defined above; and
R₆ and R₇ are defined above.

In certain embodiments, ring A is represented by the following Formula(s):

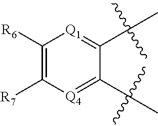

wherein Q₁ and Q₄ are N, N—O; Q₁ is N, N—O; Q₄ is CR₅; Q₁ is CR₅, Q₄ is N, N—O; or Q₁ and Q₄ are CR₅; and wherein R₆ and R₇ are defined above.

In certain embodiments, ring A is represented by the following Formula(s):

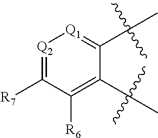

wherein:
   Q₁ and Q₂ are N; and
   R₆ and R₇ are defined above.

In certain embodiments, ring A is represented by the following Formula(s):

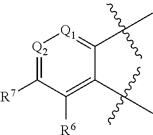

wherein:
   Q₁ is CR₅, and Q₂ is N;
   R₆ and R₇ are defined above.

In certain embodiments, ring A is represented by the following Formula(s):

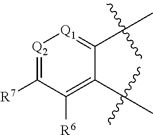

wherein Q₁ is N; and Q₂ is CR₅; and R₆ and R₇ are defined above.

In certain embodiments, ring A is selected from the group consisting of

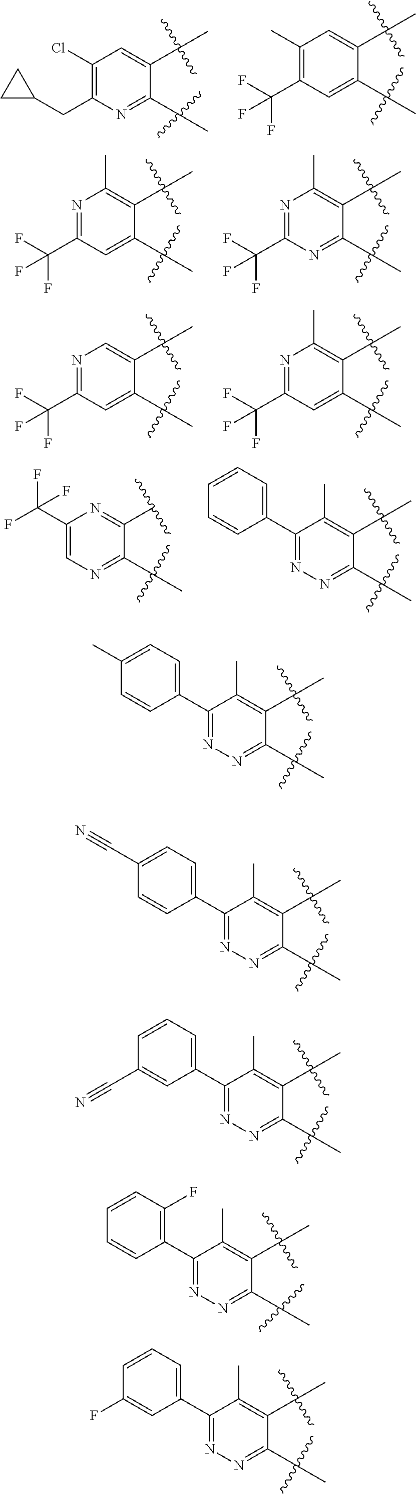

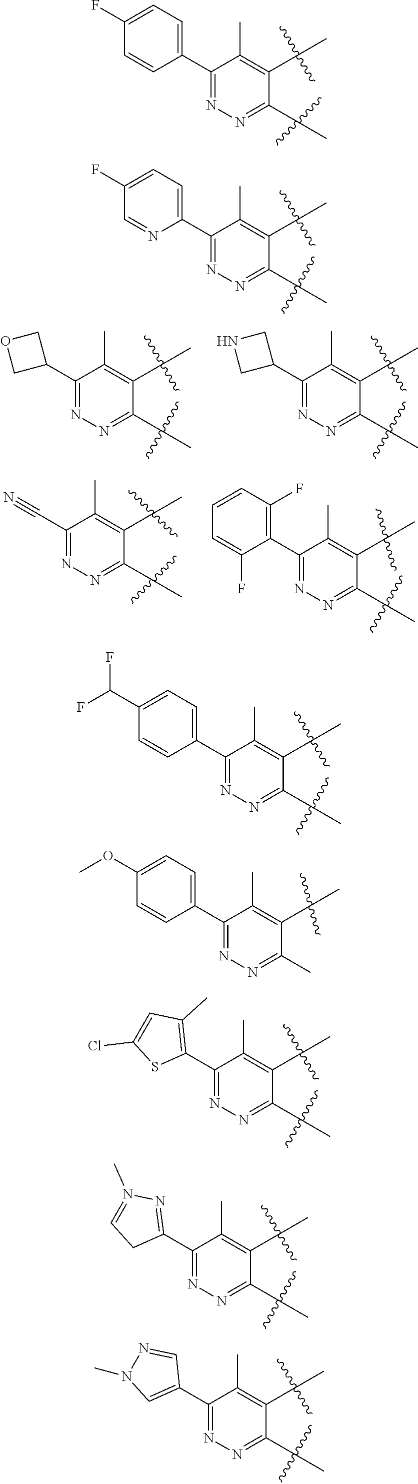

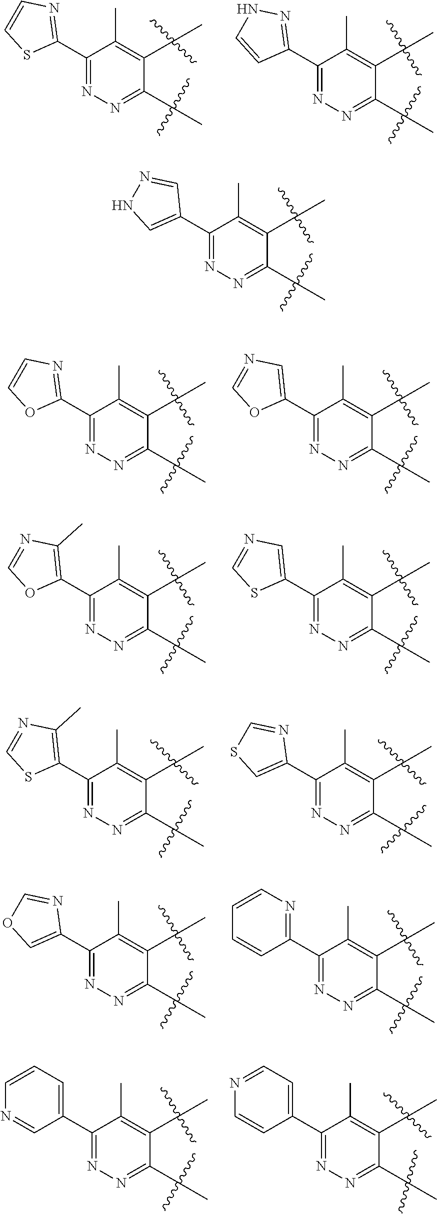

In certain embodiments, ring B is selected from the group consisting of pyrrolidine, azetidine, piperidine, piperazine, azepane, azocane, morpholine, thiomorpholine, oxazepane, isoindoline, dihydroisoquinoline, octahydroisoindole, azabicyclo[2.2.1]heptane, azabicyclo[3.1.1]heptane, azabicyclo[4.1.0]heptane, azabicyclo[3.2.1]octane, diazabicyclo-[3.2.1]octane, azabicyclo[3.2.0]heptane, oxa-azabicyclo[3.2.1]octane, azaspiro[2.5]octane, azaspiro[2.6]nonane, azaspiro[3.5]nonane, oxa-azaspiro[3.5]nonane, oxa-azaspiro[4.5]decane, dihydrothieno[3,2-c]pyridine, dihydrothiazolo[4,5-c]pyridine, dihydrooxazolo[4,5-c]pyridine, dihydroimidazo[1,2-a]pyrazine, hexahydrofuro[3,2-b]pyrrole, hexahydrocyclopenta[c]pyrrole, and azatricyclo[4.3.1.13,8]undecane.

In certain embodiments, ring C is phenyl.

In certain embodiments, ring C is pyridyl.

In certain embodiments, in Formula (II), X₁ and X₂ are O.

In certain embodiments, in Formula (II), X₁ is O and X₂ is NH.

In certain embodiments, the compound of Formula (I) is further described as Formula (III):

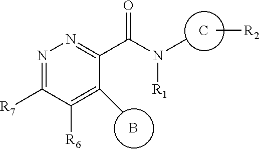

wherein:
   A, B, C, R₁, and R₂ are defined above, and R₆ and R₇ are defined above.

In certain embodiments, substituted or unsubstituted B is selected from a group consisting of:

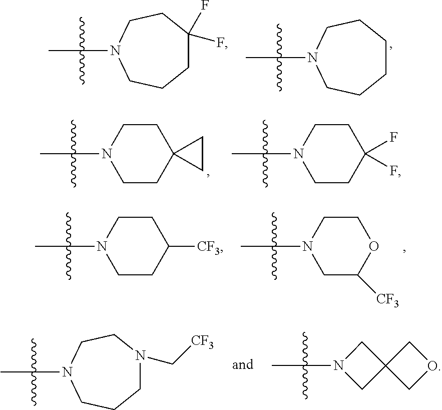

In certain embodiments, substituted or unsubstituted C is selected from a group consisting of:

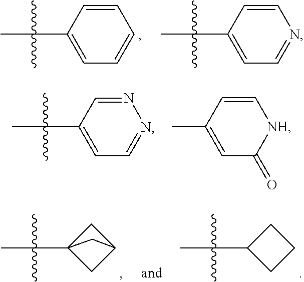

In certain embodiments, R₁ is H.

In certain embodiments, R₂ is selected from a group consisting of: H,

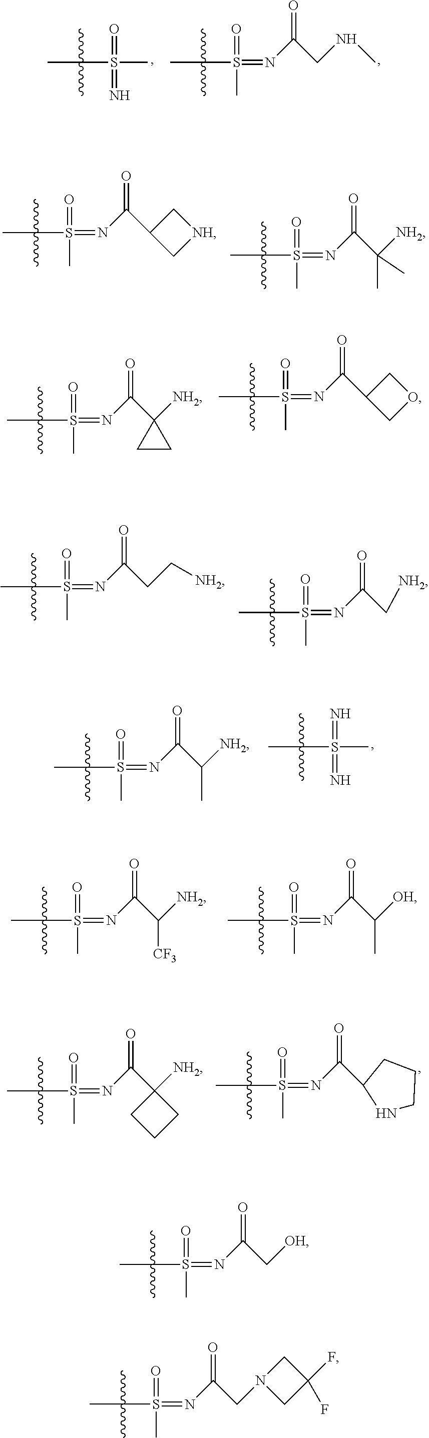

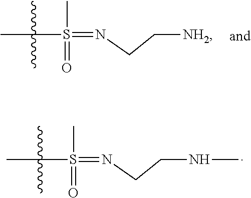

In certain embodiments, R₂ is selected from a group consisting of: H,

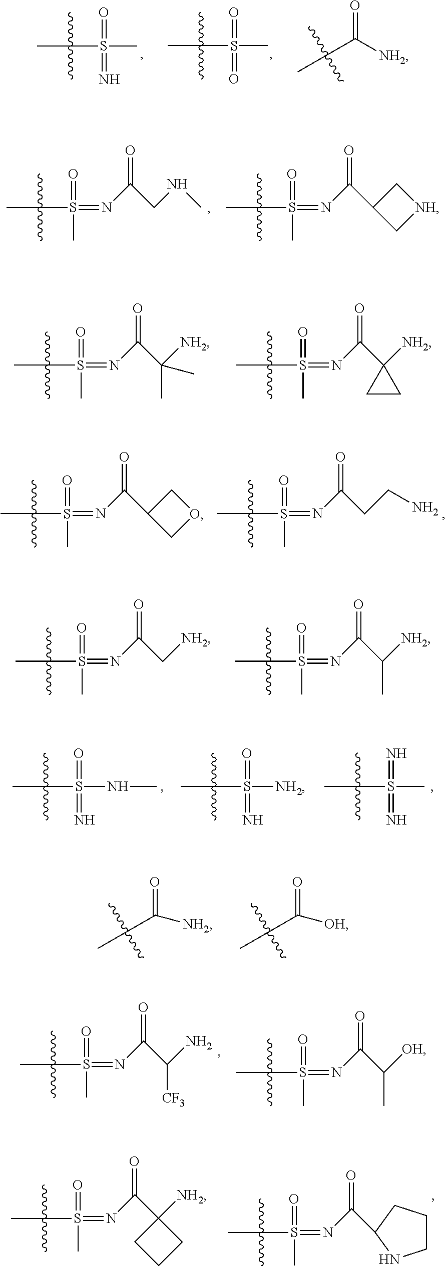

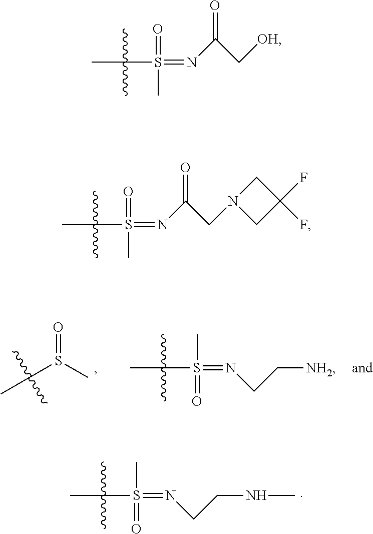

In certain embodiments, R₂ is:

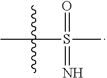 [SMILES: *S(C)(=N)=O]

In certain embodiments, ring C comprises additional substitutions, i.e., substitutions in wherein the substitutions are selected from a group consisting of H, halo, and alkyl. In certain embodiments, the said halo substitution in ring C is F.

In certain embodiments, the one or more substitutions on the ring A ring are selected from a group consisting of: halo, cyano, haloalkyl, cyanoalkyl, substituted or unsubstituted C₁-C₆ alkyl, aryl, C₃-C₆ cycloalkyl, C₃-C₆ heterocycloalkyl, C₃-C₆ heteroaryl, and a combination thereof, wherein the heterocycloalkyl and heteroaryl comprise one or more hetero atoms selected from a group consisting of: N, O, or S.

In certain embodiments, the one or more substitutions on the ring A are selected from a group consisting of: methyl, trifluoromethyl, chloro, fluoro, bromo, C₁-C₆ alkyl, phenyl, cycloalkyl, methyl pyrazole, and fused 1,4-dioxane.

In certain embodiments, the one or more substitutions on the ring A are selected from a group consisting of: —CH₃, —CF₃, —Cl, —Br, —F, —CH₂—CH₂—CH═CH₂, phenyl, —CH₂—CN, —C(O)—NH₂,

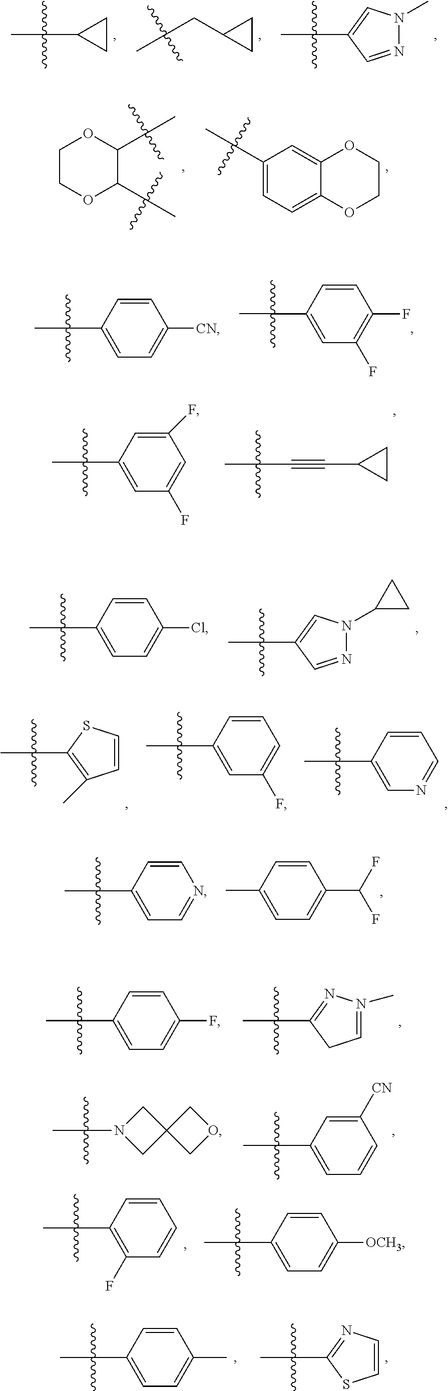

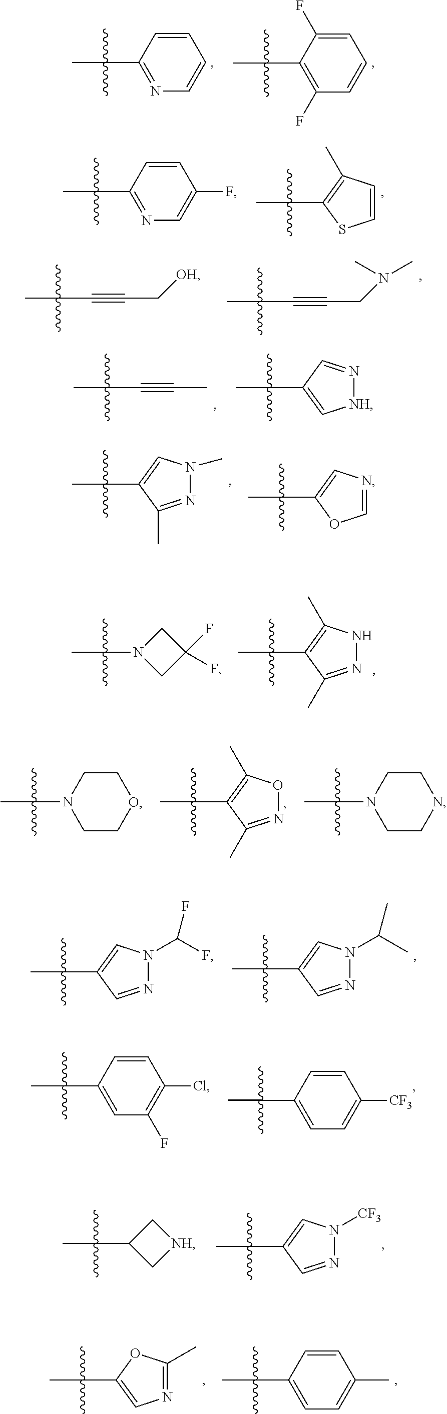

and any combination thereof.

In certain embodiments, the compound of Formula (I) is selected from the group consisting of:

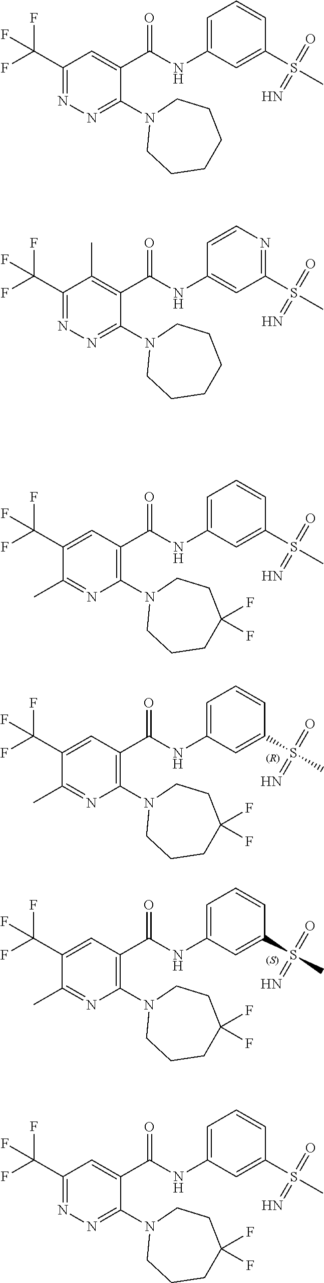

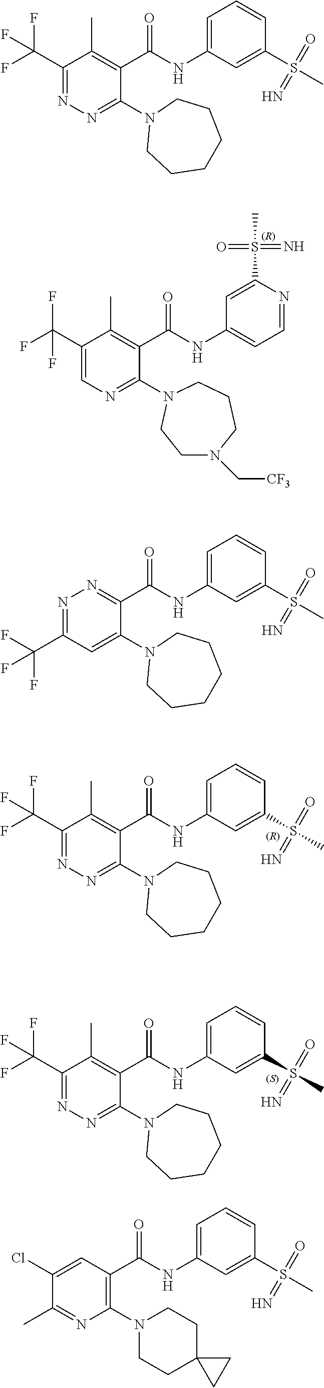

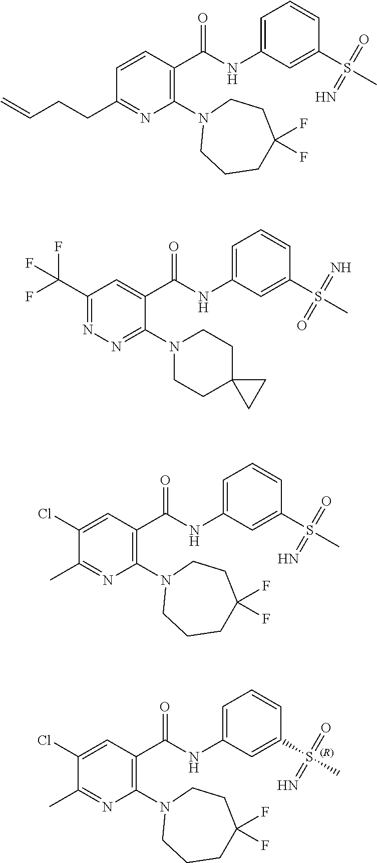

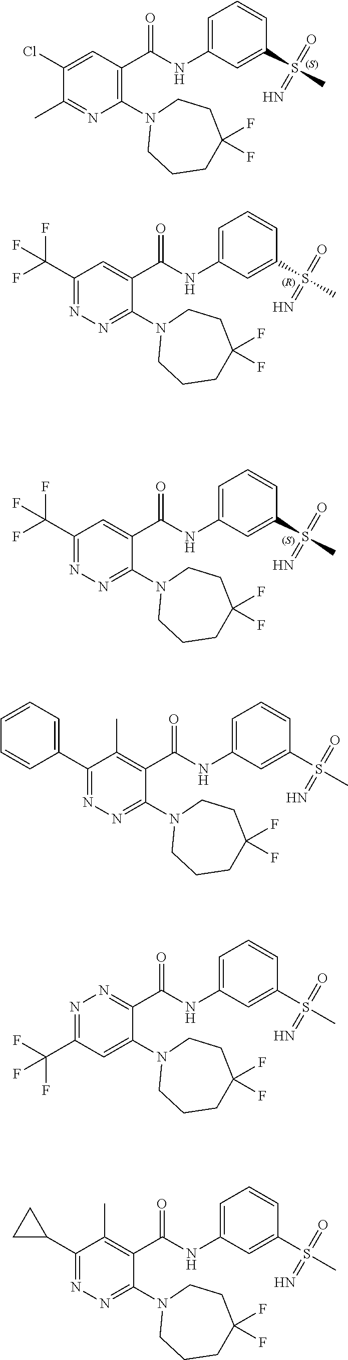

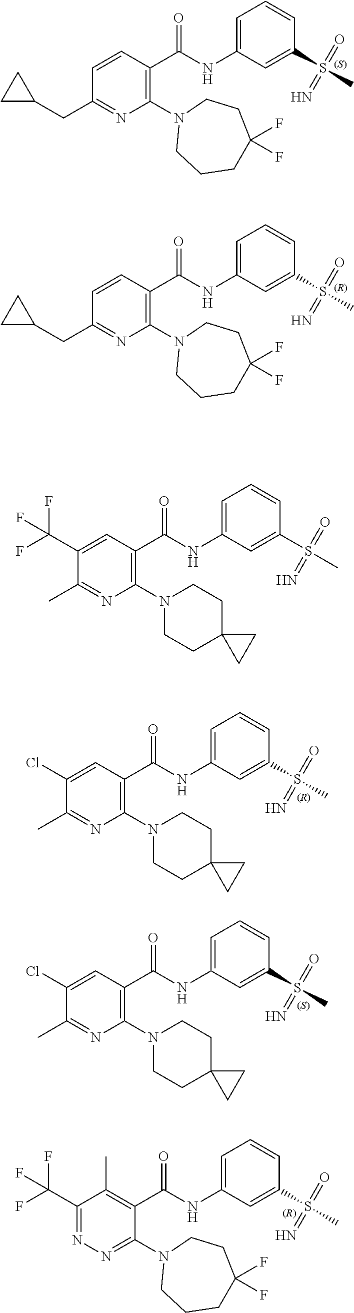

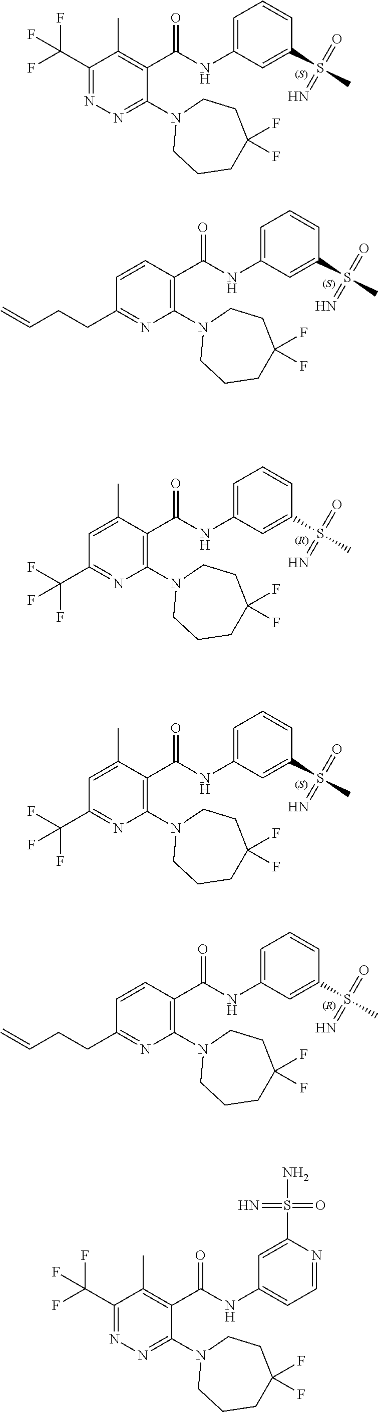

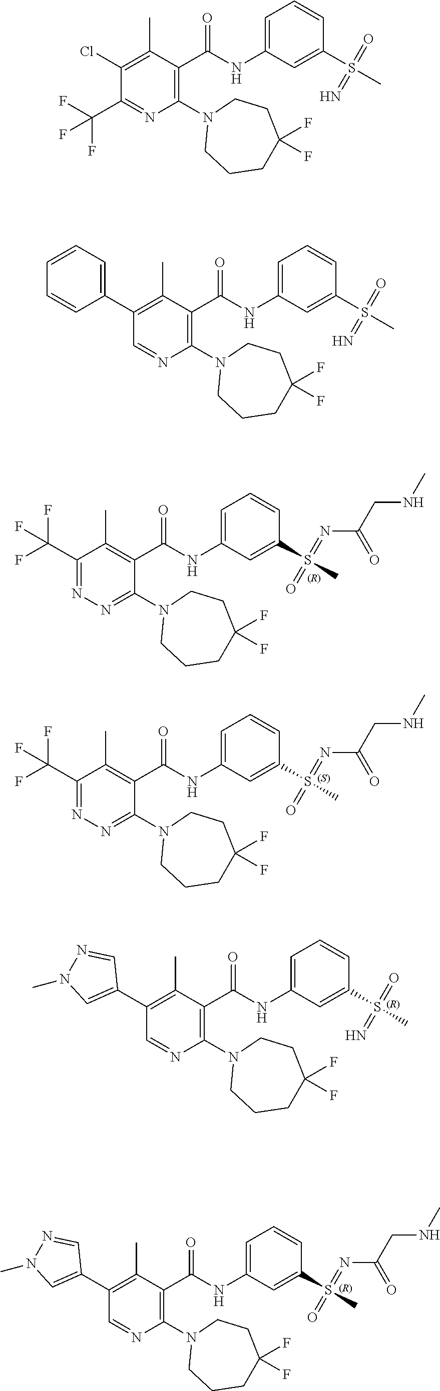

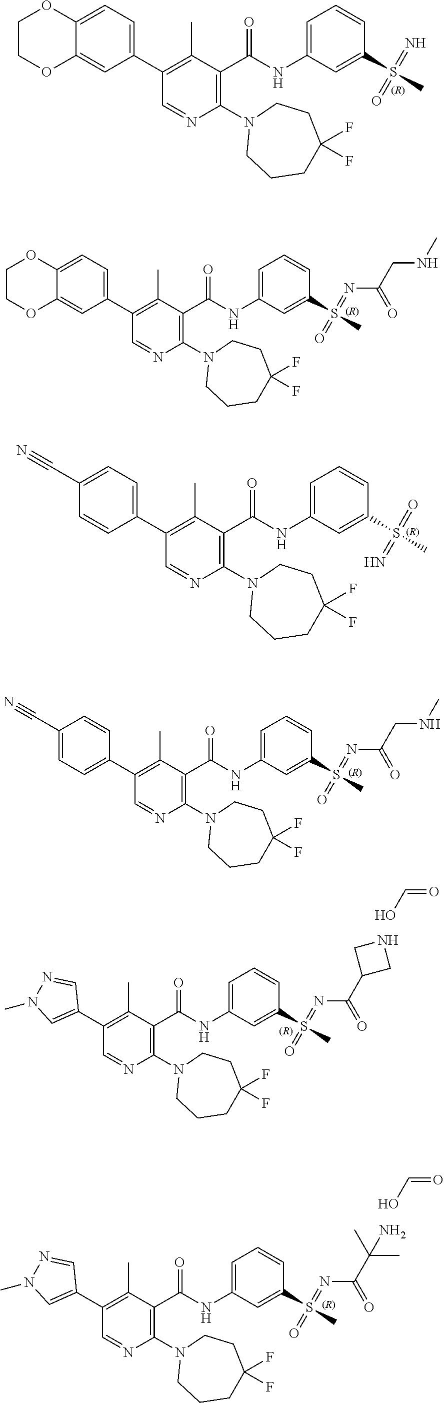

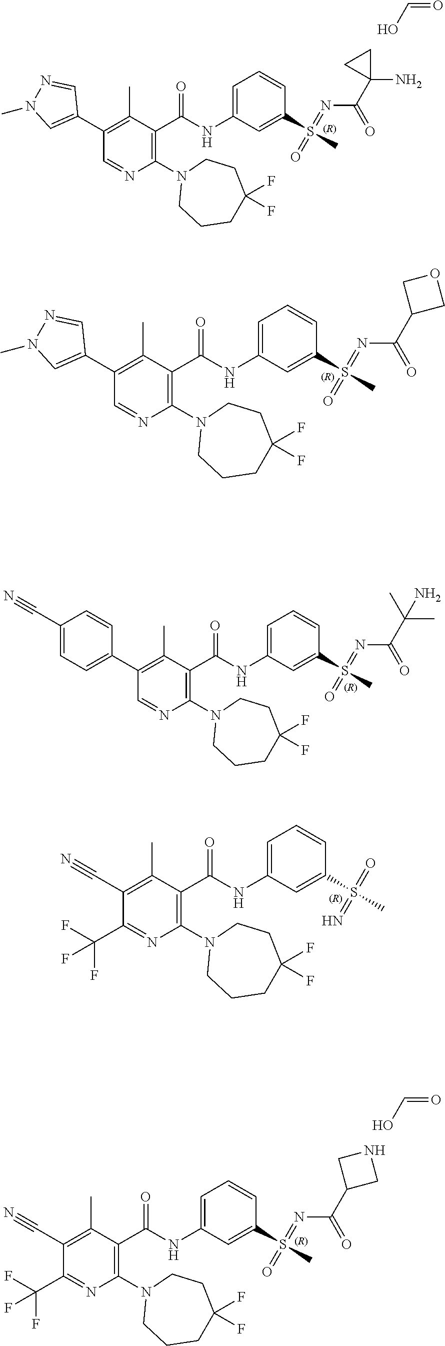

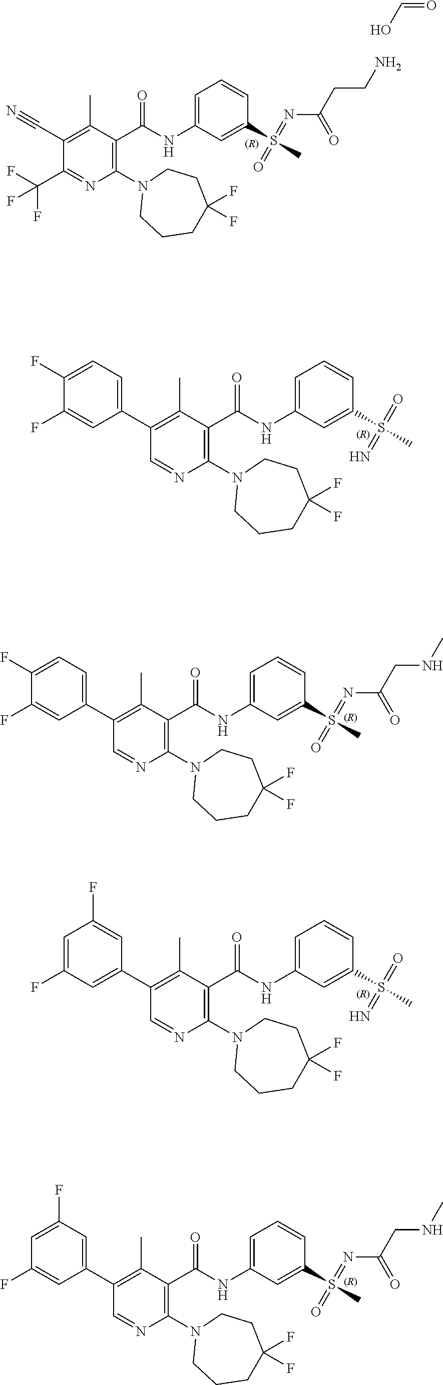

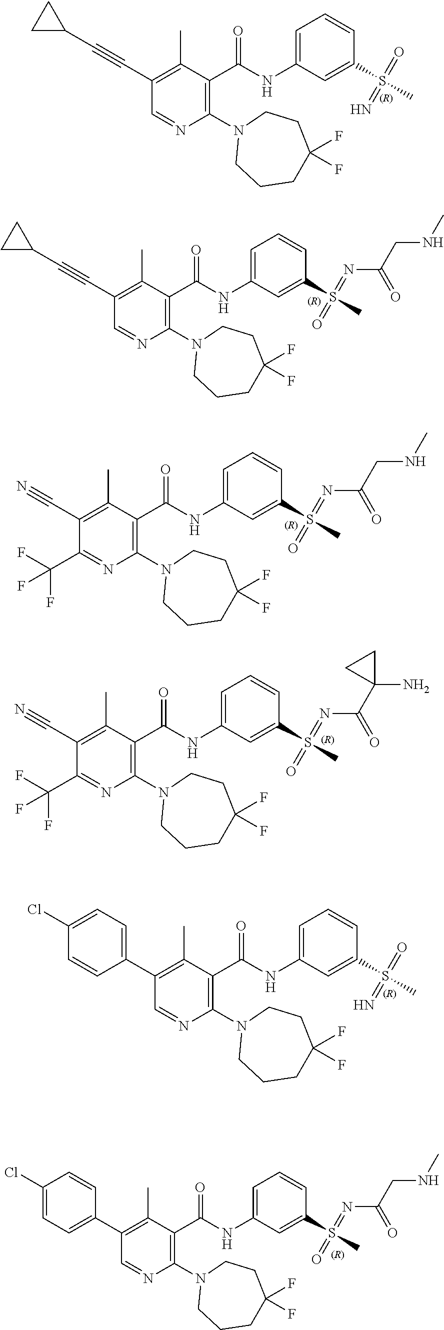

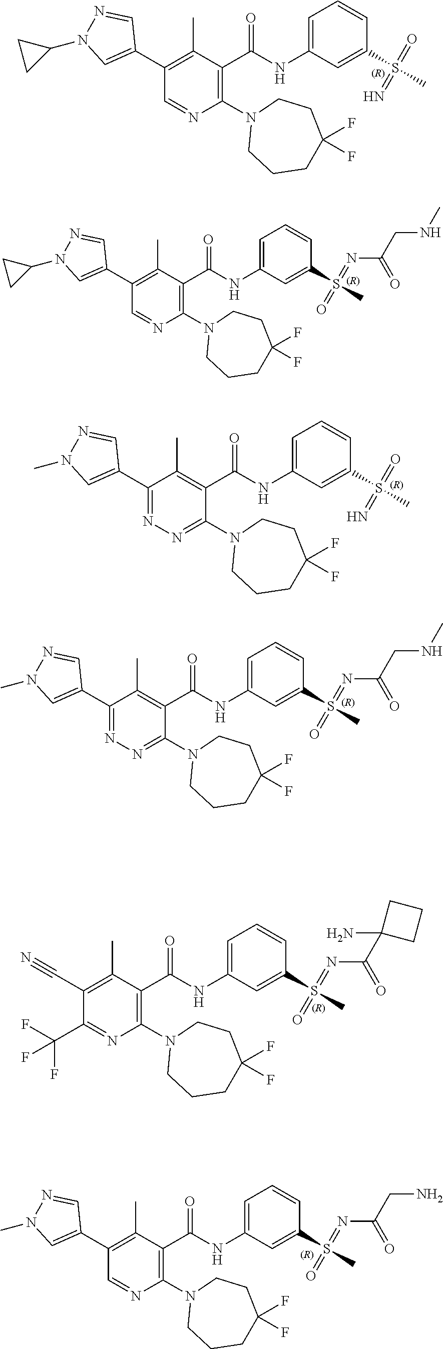

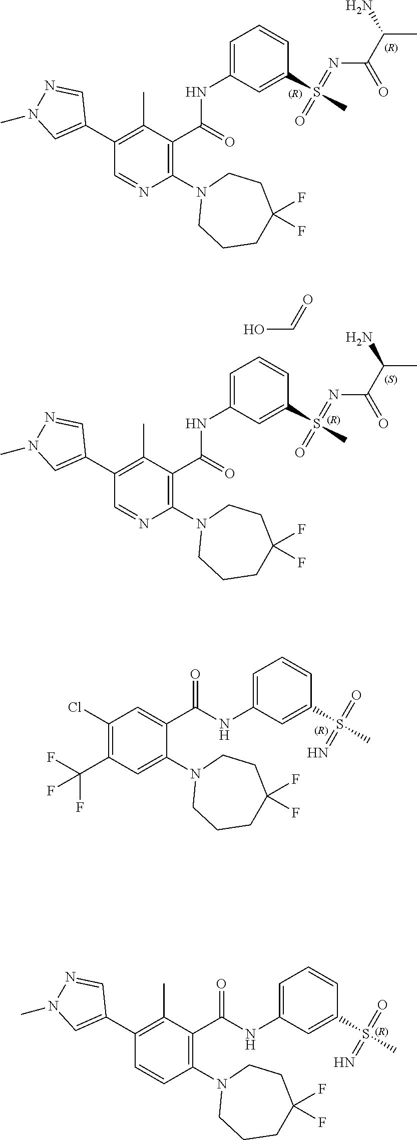

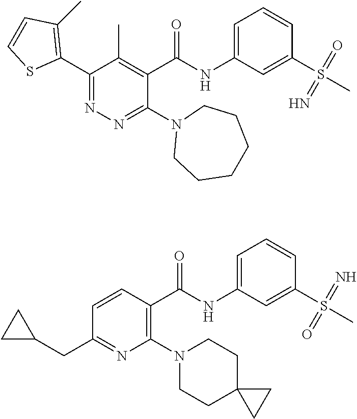 [SMILES: Cc1ccsc1-c1nnc(N2CCCCCC2)c(C(=O)Nc2cccc(S(C)(=N)=O)c2)c1C ; CS(=N)(=O)c1cccc(NC(=O)c2ccc(CC3CC3)nc2N2CCC3(CC2)CC3)c1]

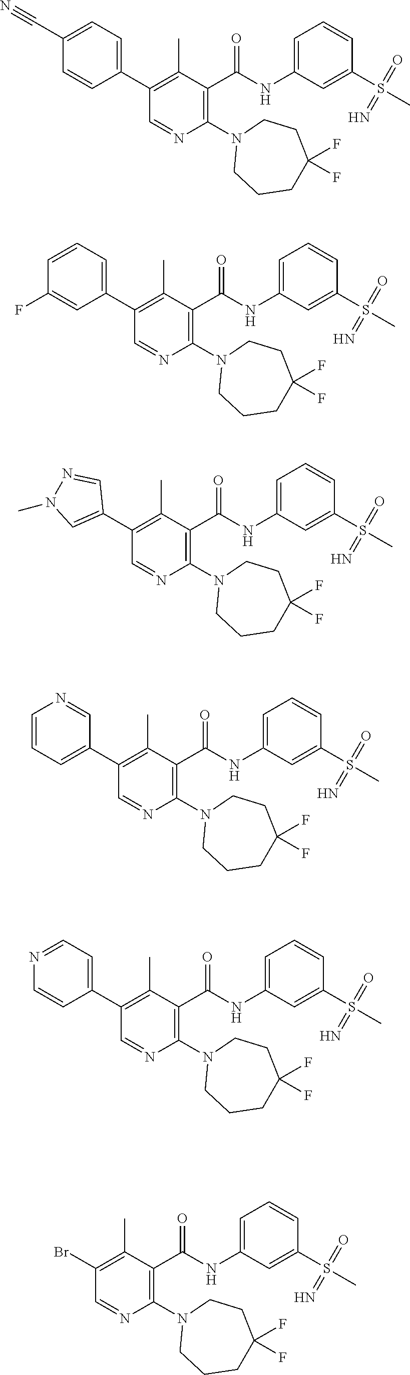

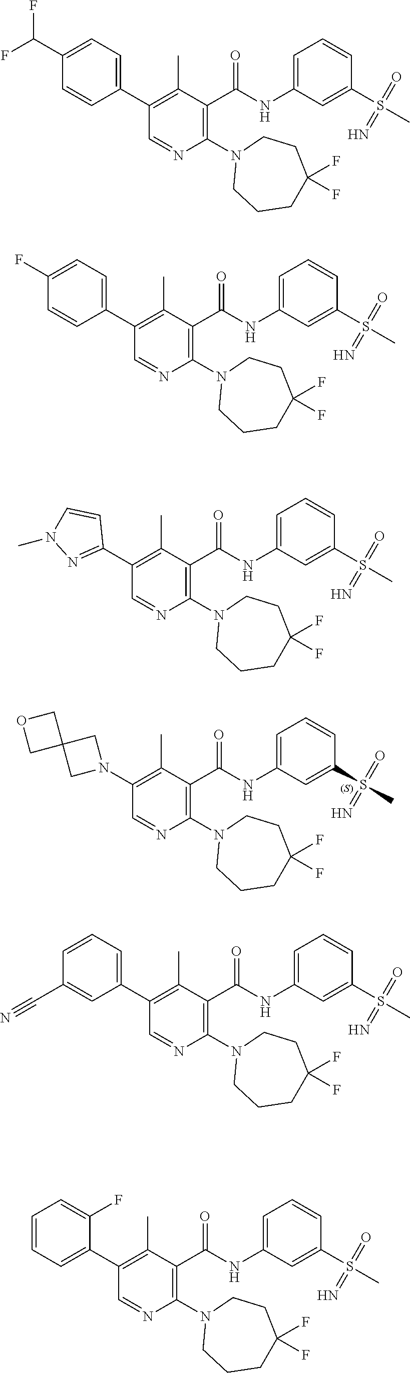

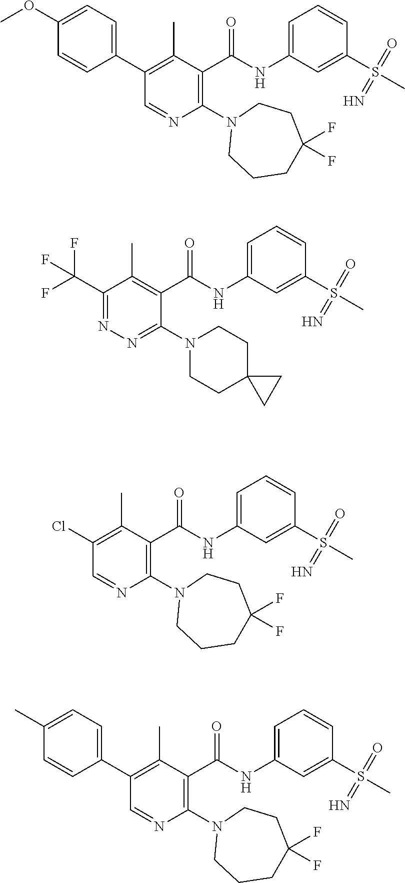

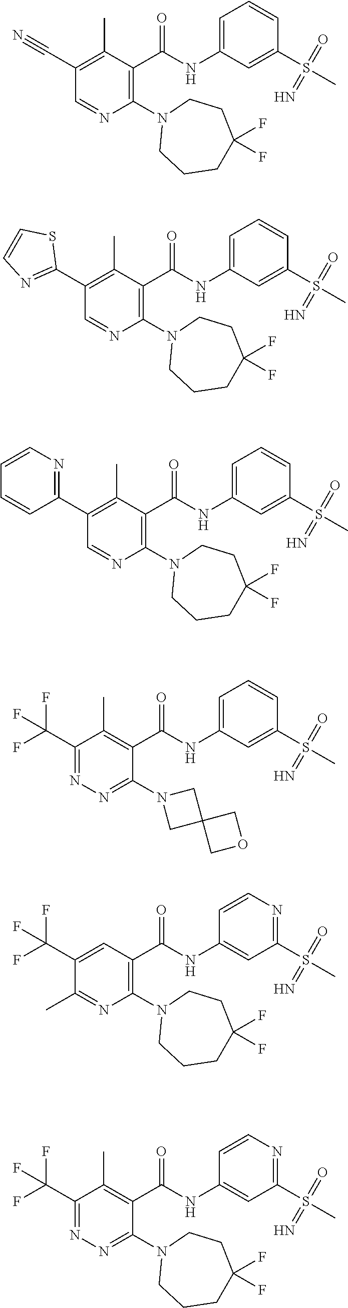 [SMILES: Cc1c(C#N)cnc(N2CCCC(F)(F)CC2)c1C(=O)Nc1cccc(S(C)(=N)=O)c1 ; Cc1c(-c2nccs2)cnc(N2CCCC(F)(F)CC2)c1C(=O)Nc1cccc(S(C)(=N)=O)c1 ; Cc1c(-c2ccccn2)cnc(N2CCCC(F)(F)CC2)c1C(=O)Nc1cccc(S(C)(=N)=O)c1 ; Cc1c(C(F)(F)F)nnc(N2CC3(COC3)C2)c1C(=O)Nc1cccc(S(C)(=N)=O)c1 ; Cc1nc(N2CCCC(F)(F)CC2)c(C(=O)Nc2ccnc(S(C)(=N)=O)c2)cc1C(F)(F)F ; Cc1c(C(F)(F)F)nnc(N2CCCC(F)(F)CC2)c1C(=O)Nc1ccnc(S(C)(=N)=O)c1]

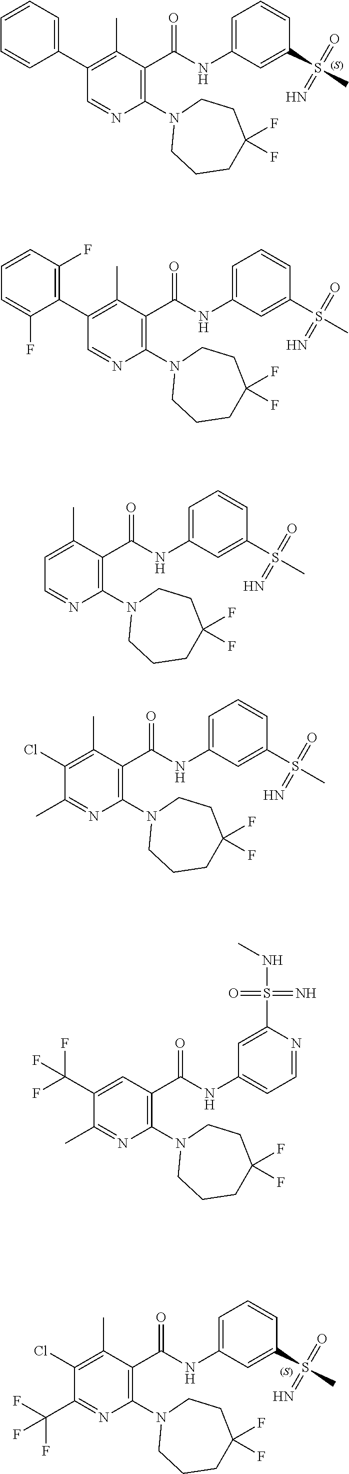

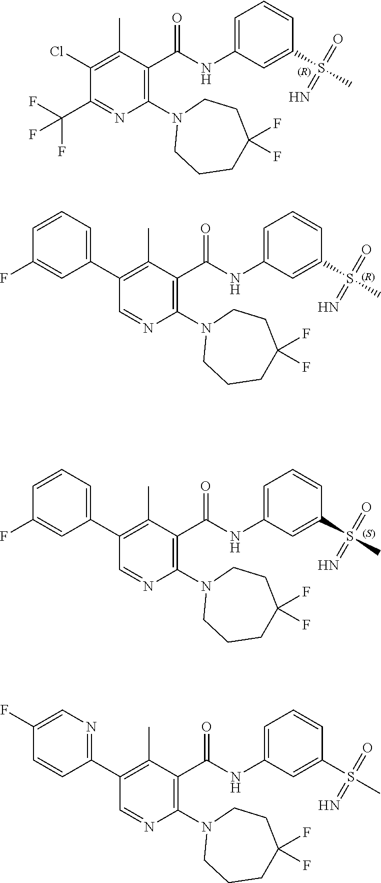

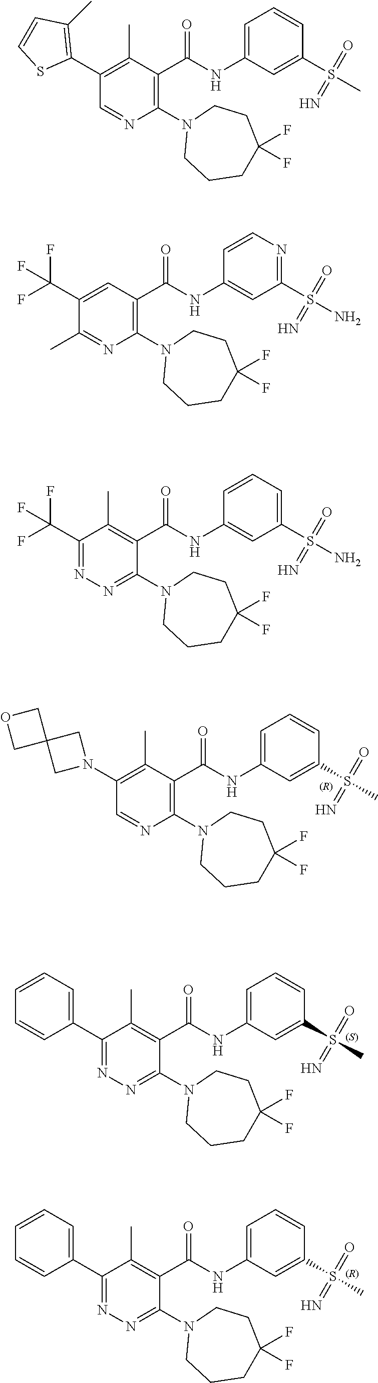

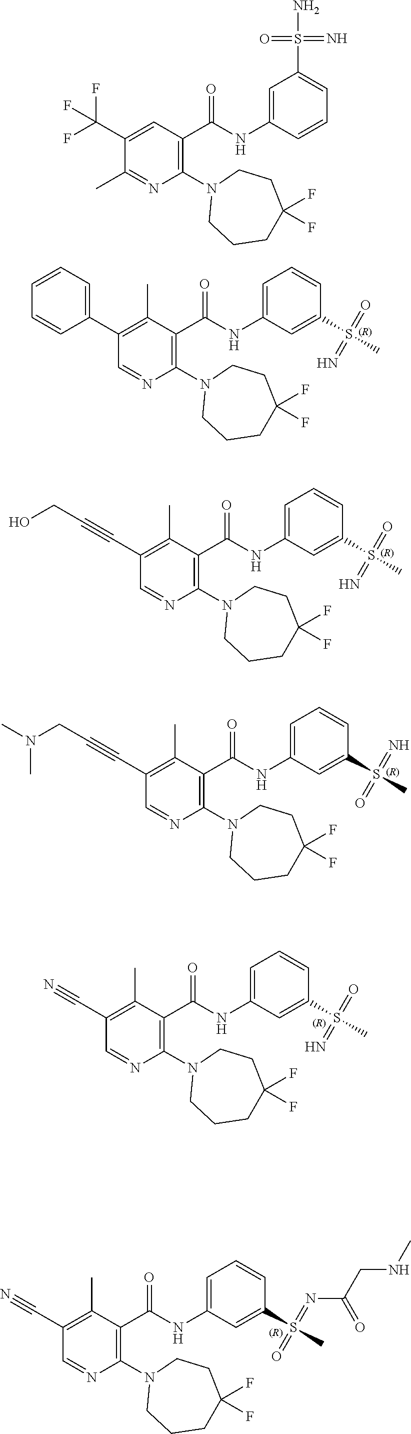

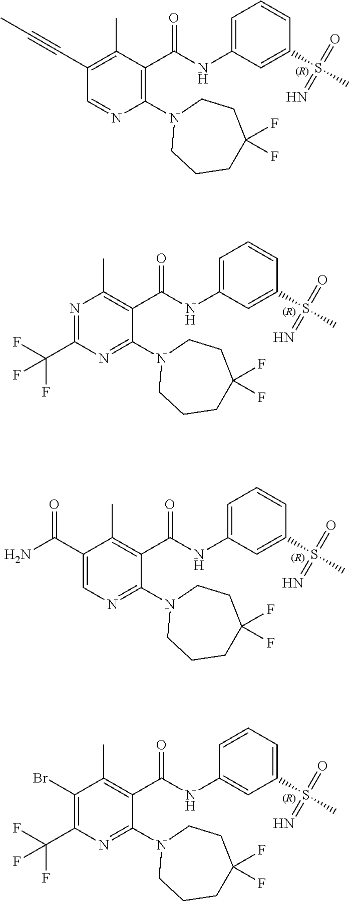

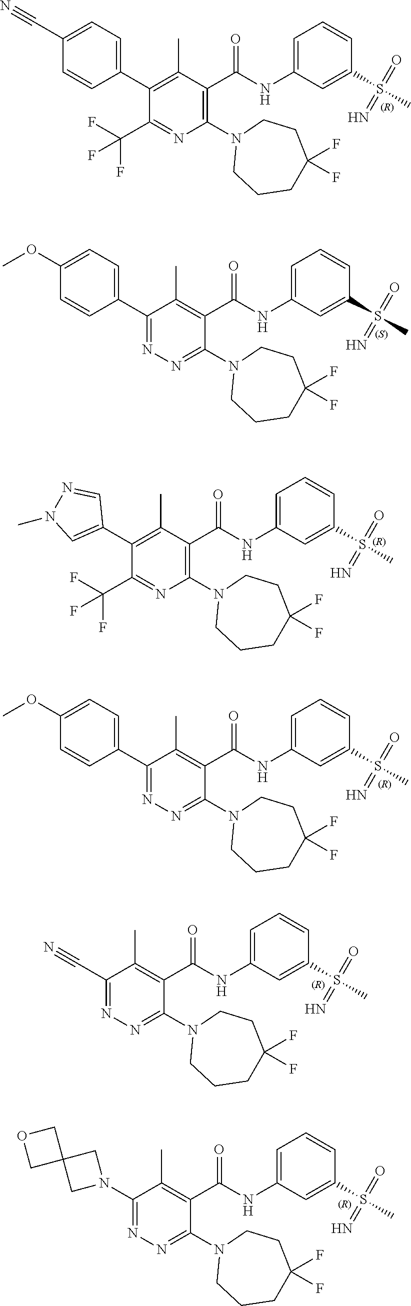

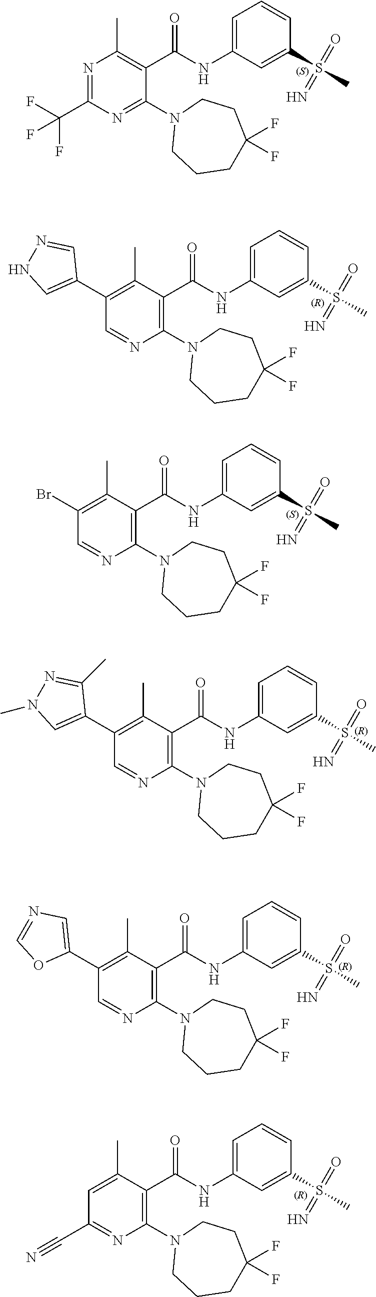

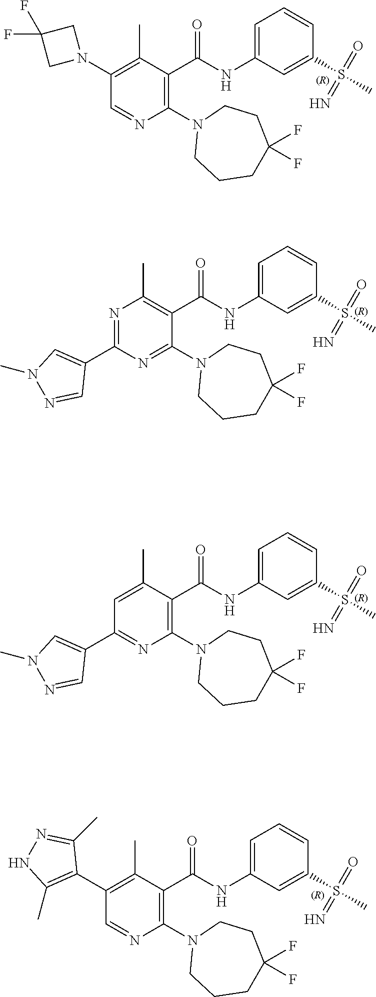

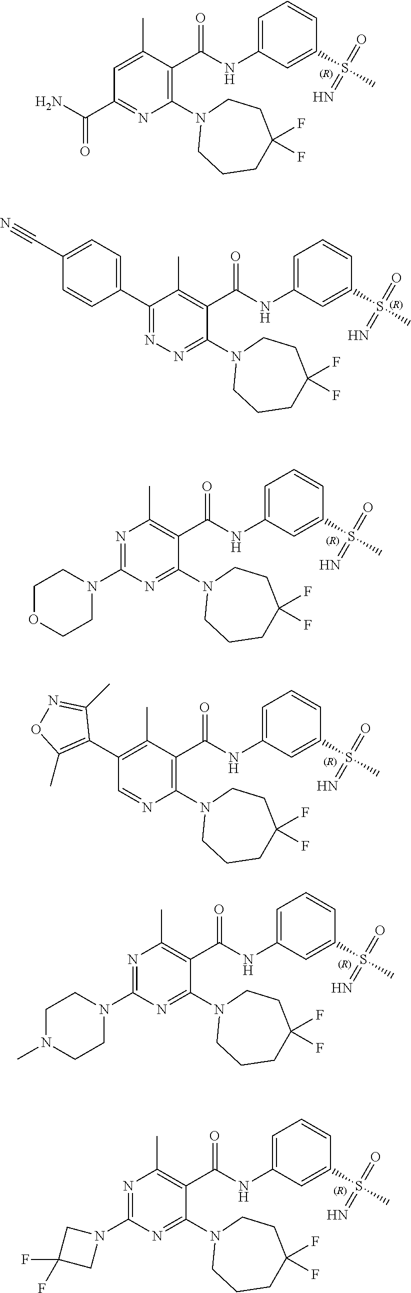

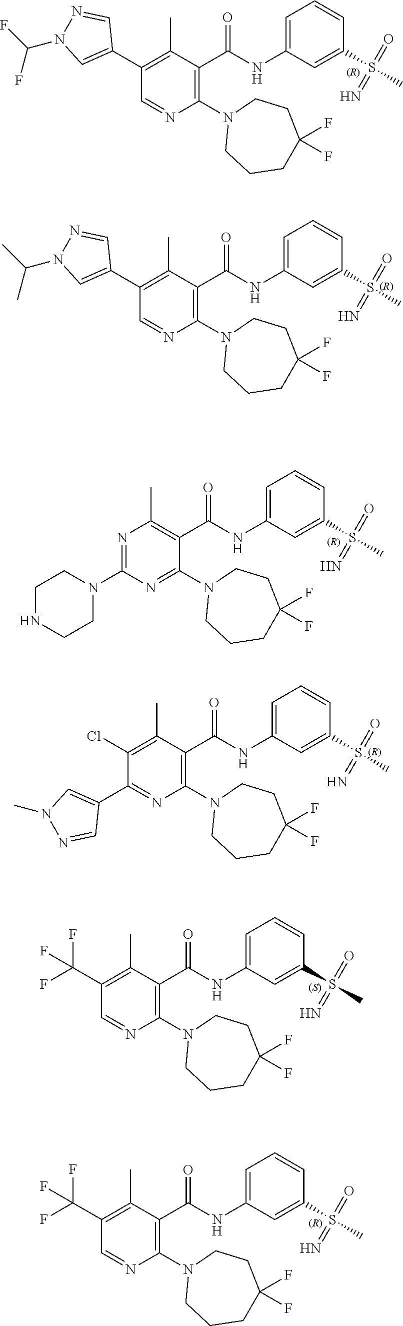

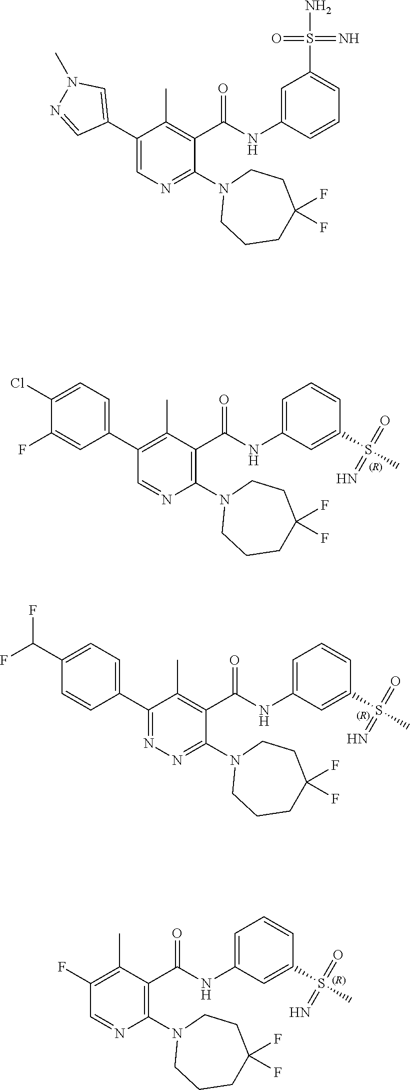

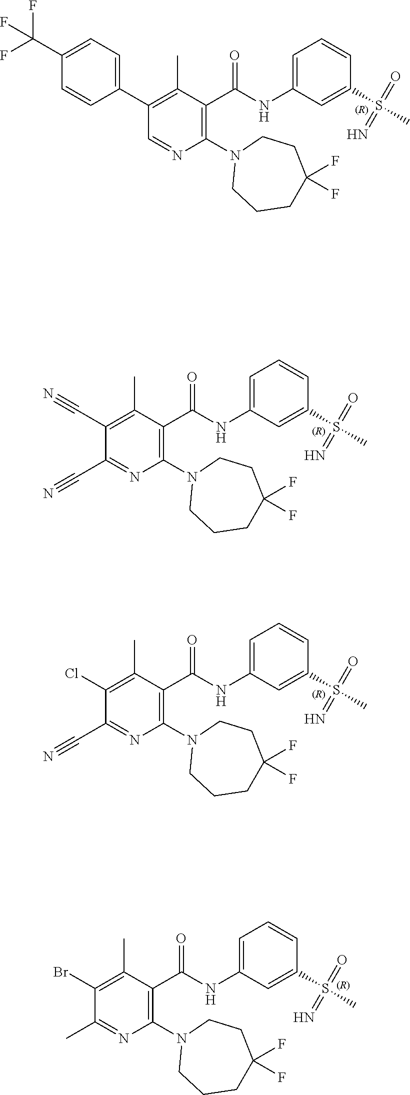

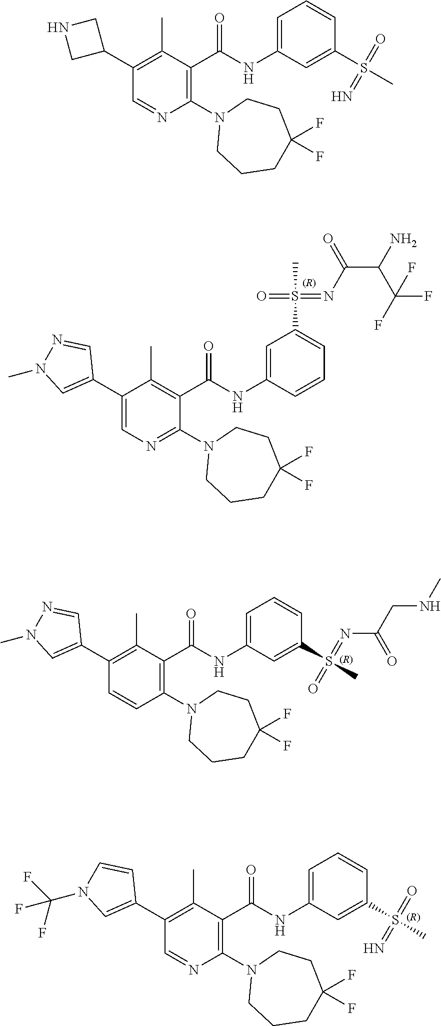

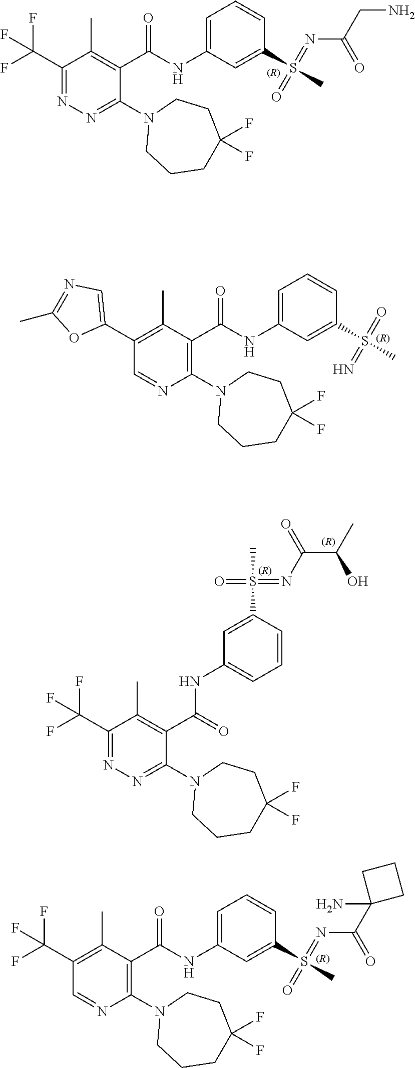

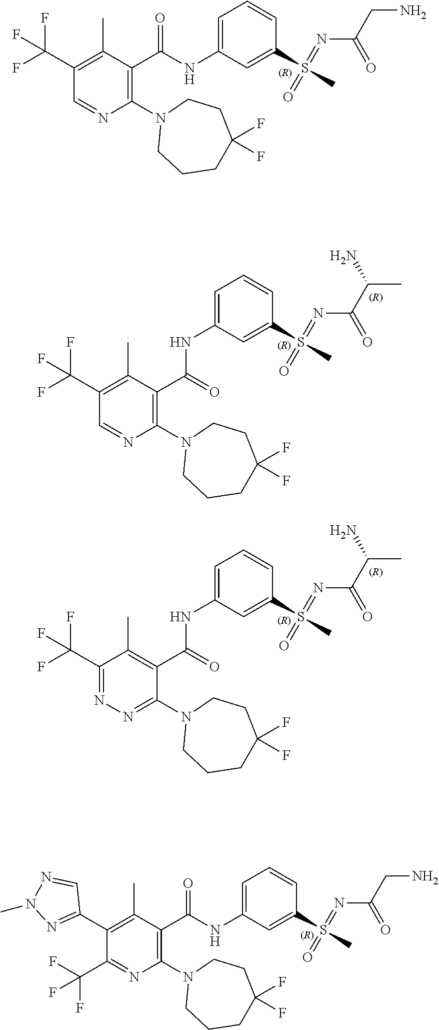

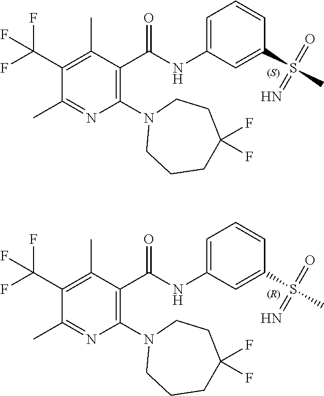

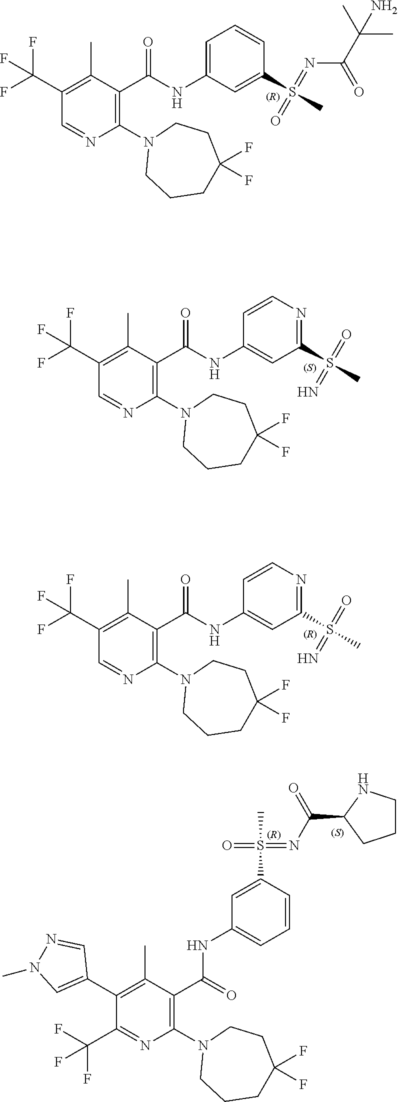

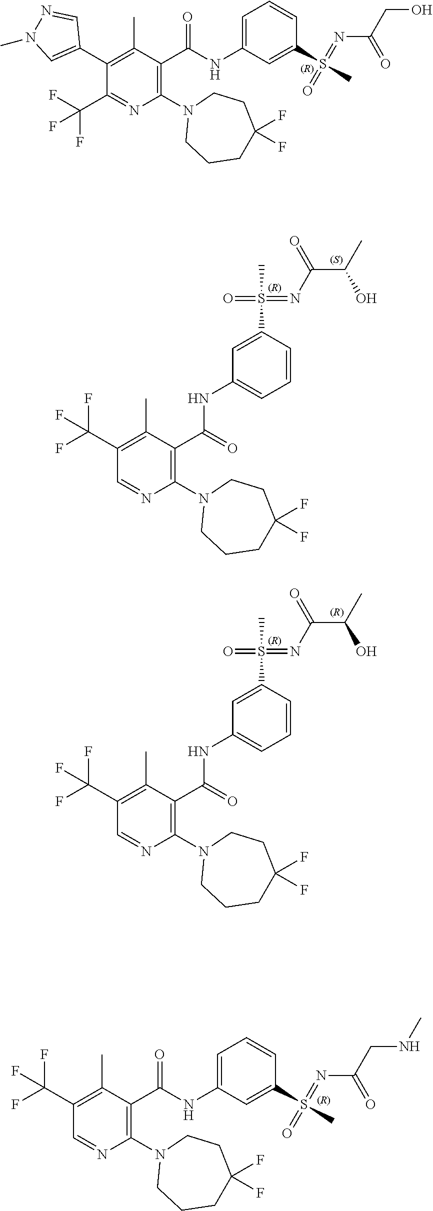

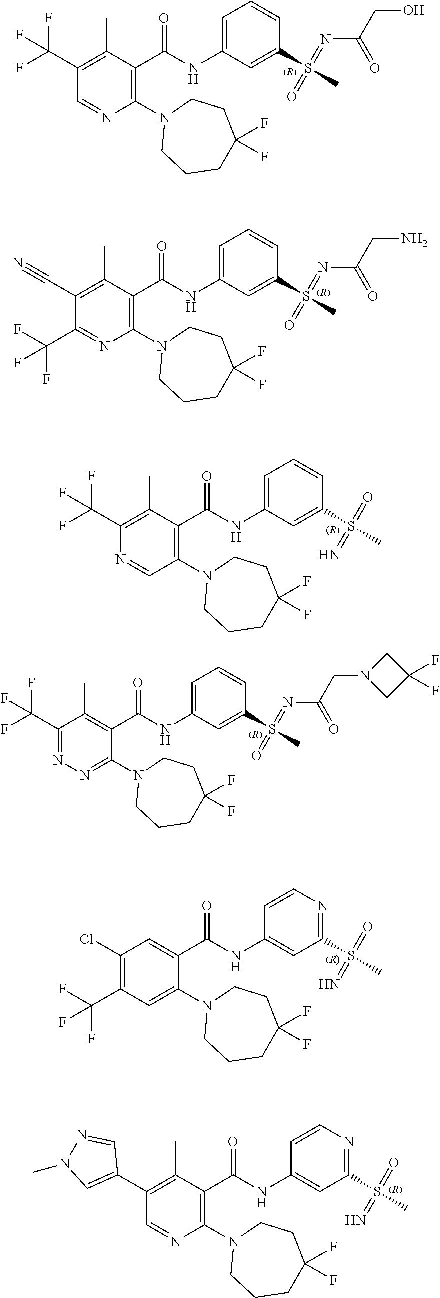

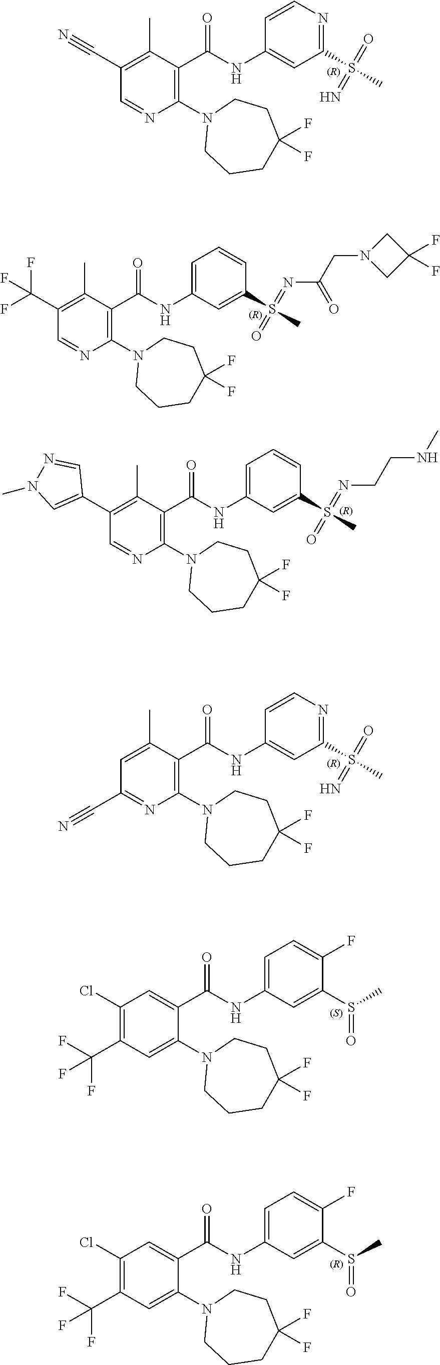

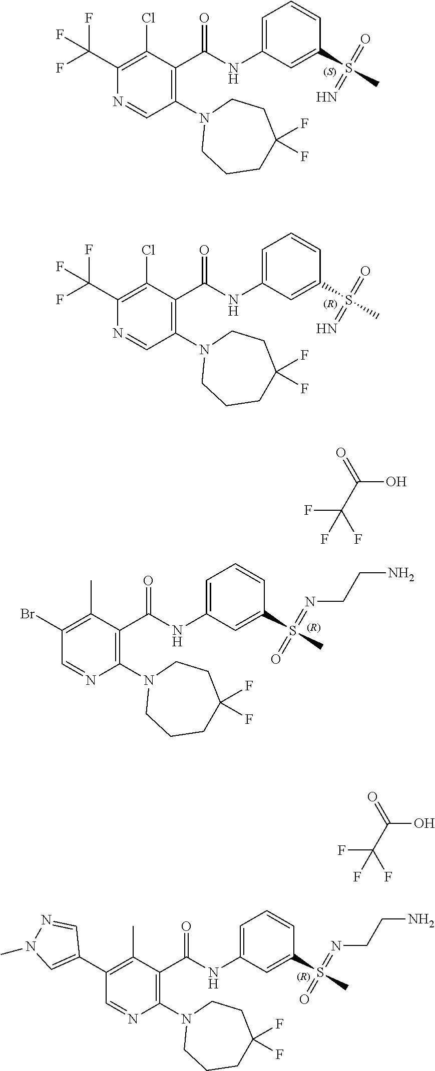

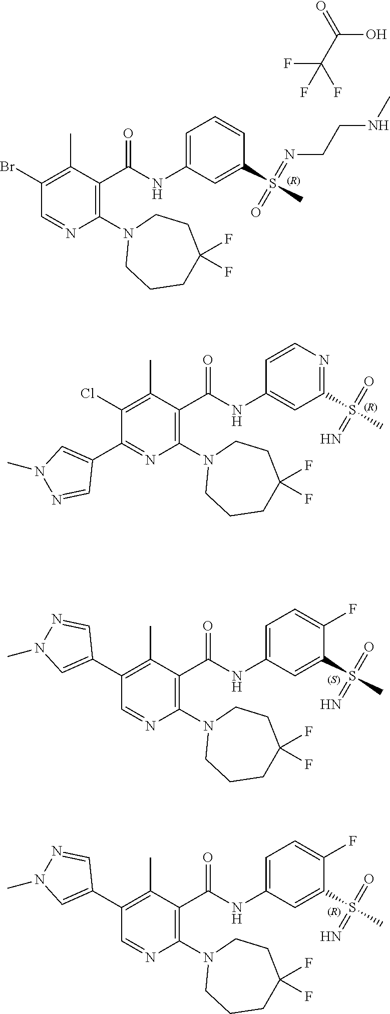

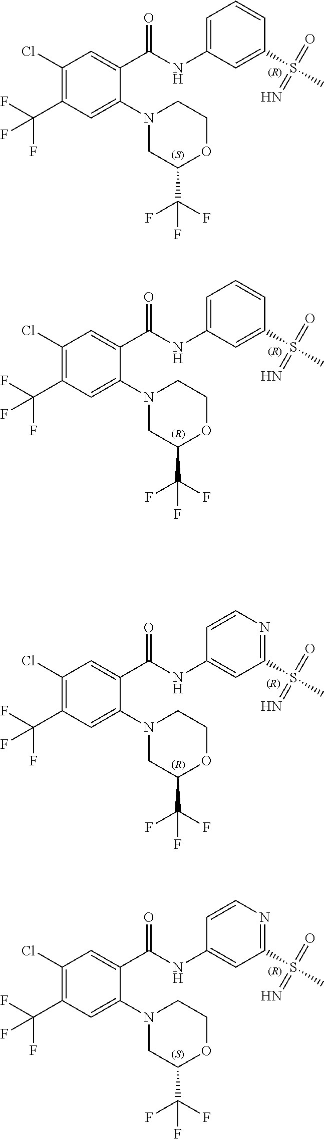

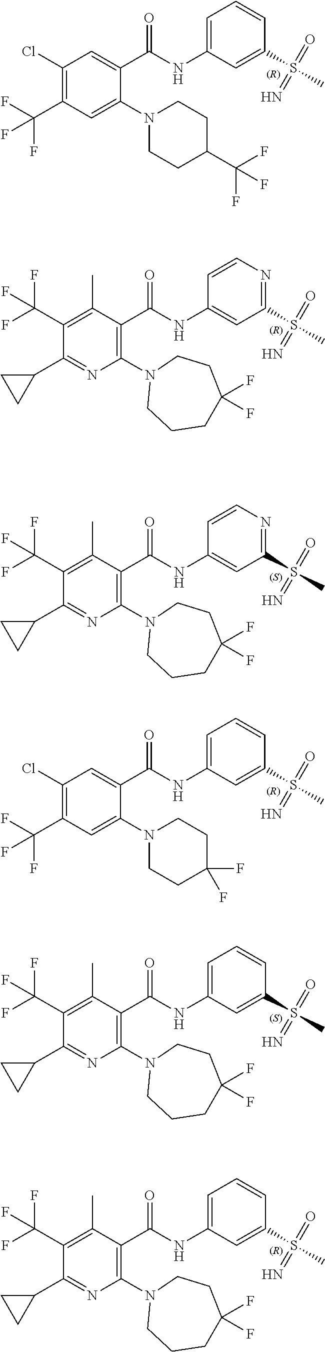

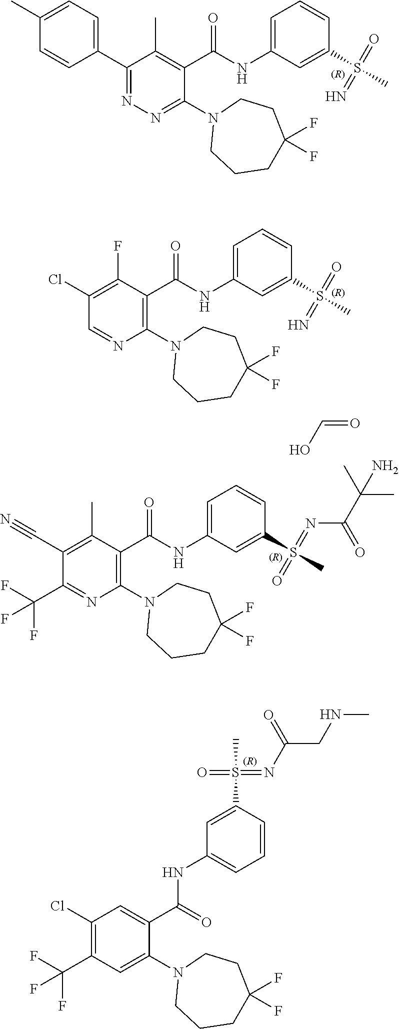

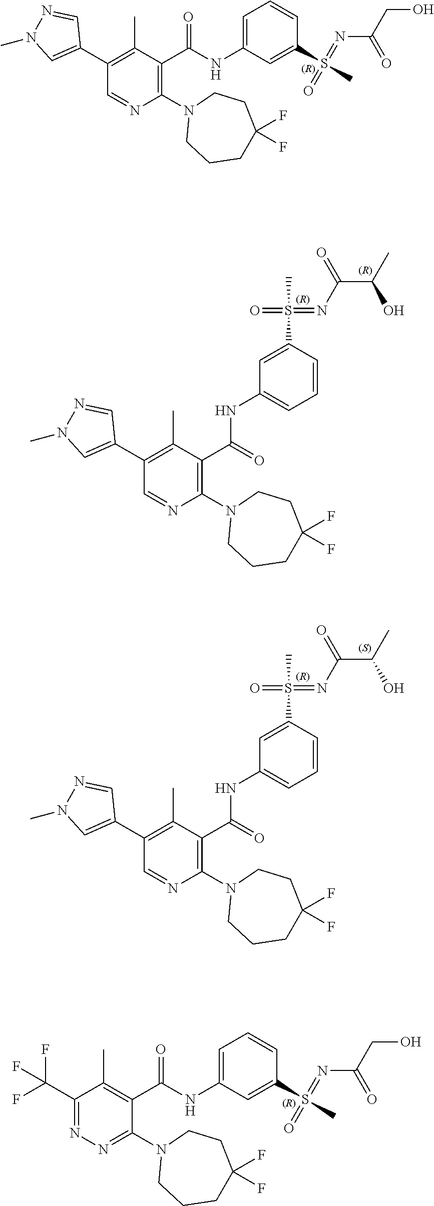

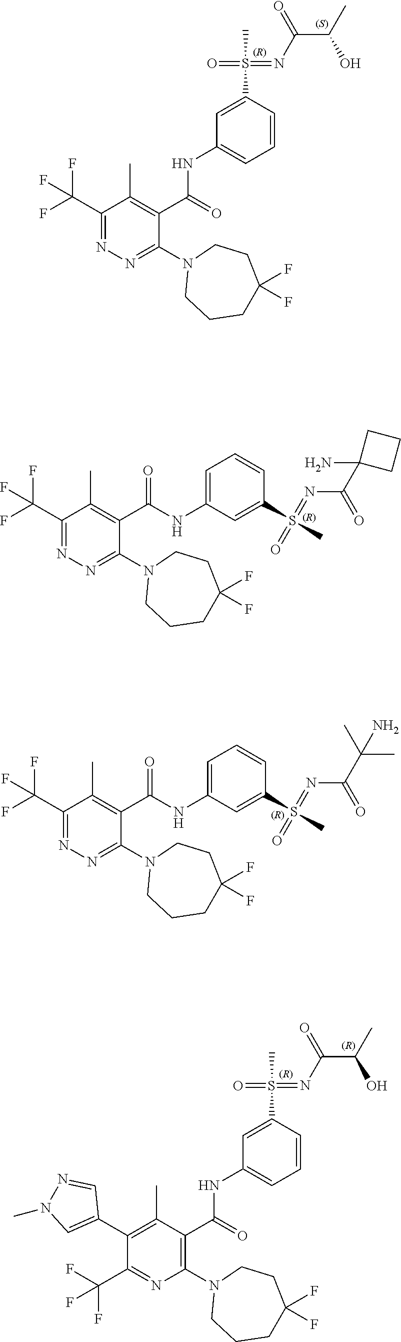

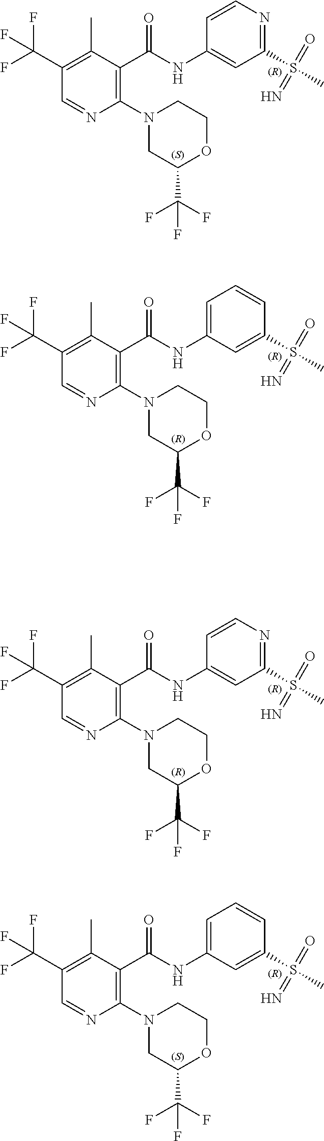

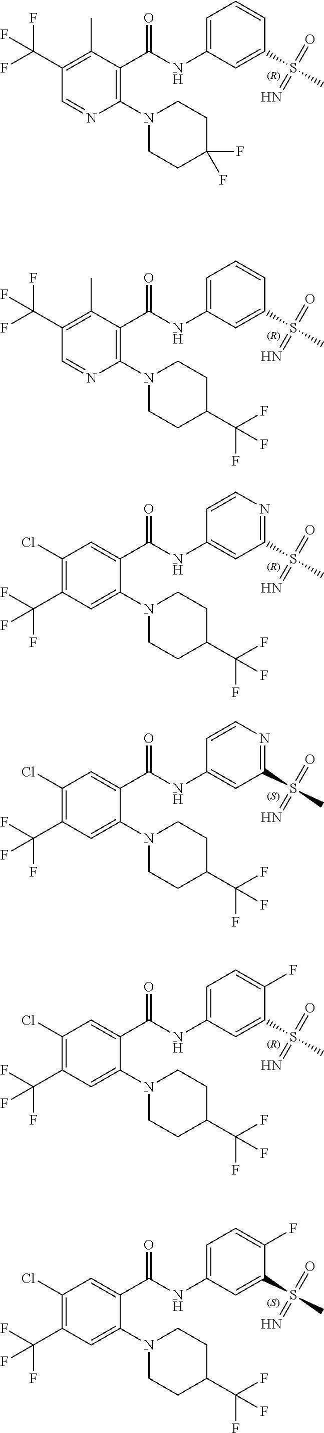

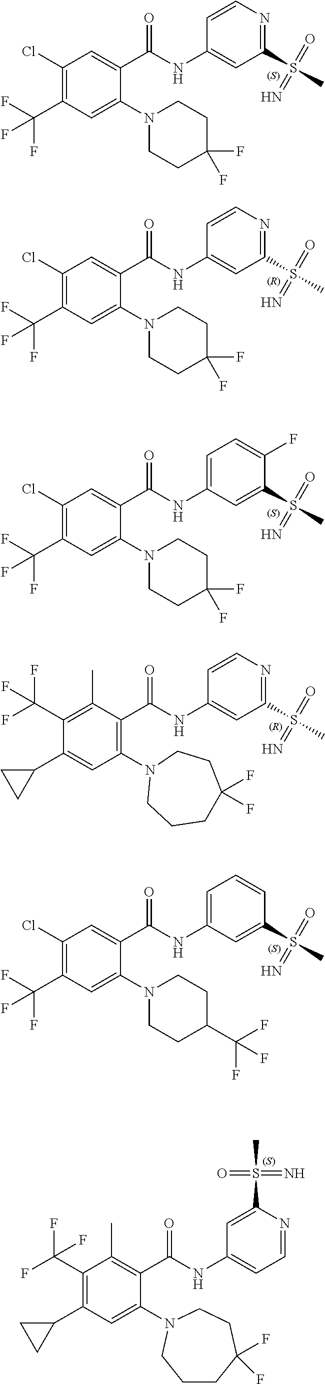

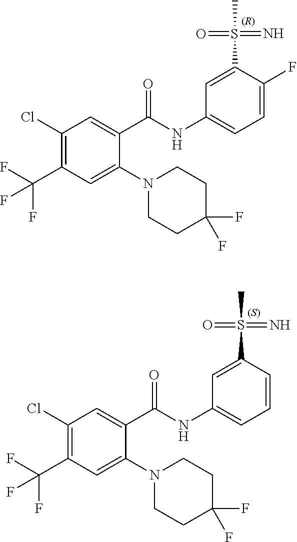

In certain embodiments, the compound of the invention is selected from a group consisting of:

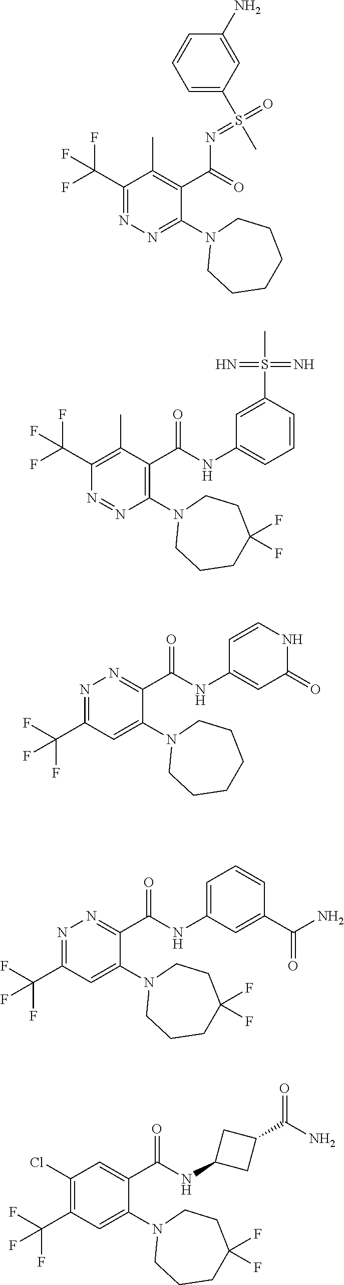

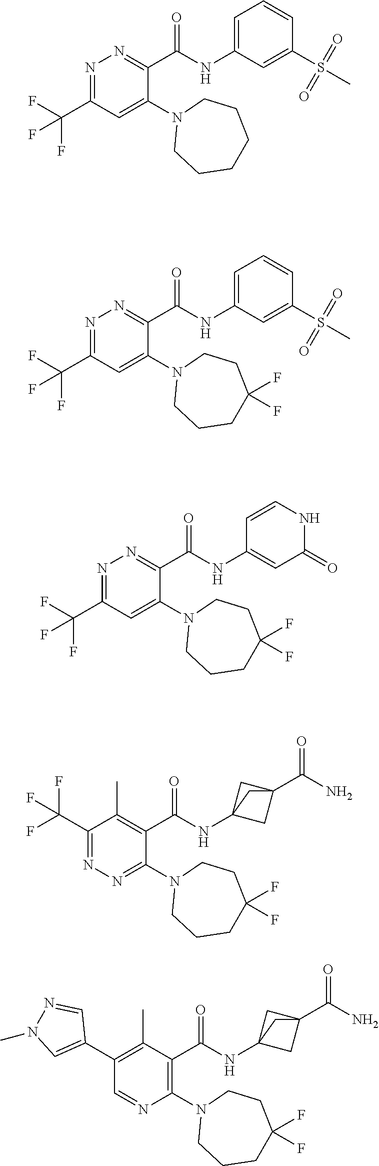

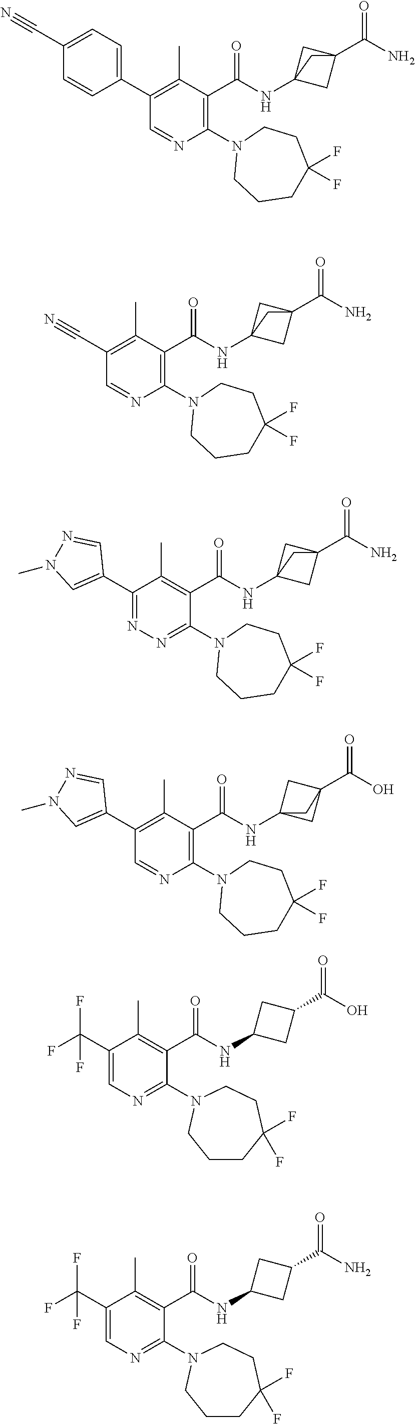

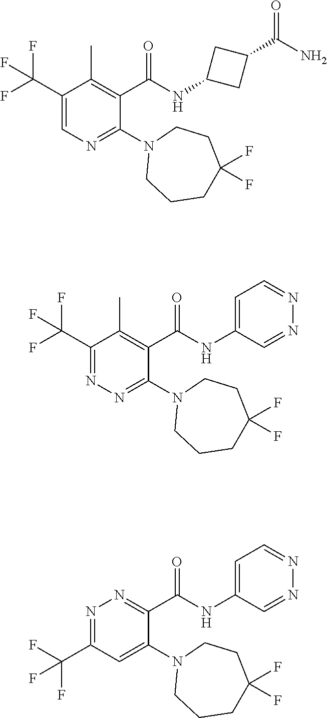 [SMILES: Cc1c(C(F)(F)F)cnc(N2CCCC(F)(F)CC2)c1C(=O)N[C@H]1C[C@@H](C(N)=O)C1 ; Cc1c(C(F)(F)F)nnc(N2CCCC(F)(F)CC2)c1C(=O)Nc1ccnnc1 ; O=C(Nc1ccnnc1)c1nnc(C(F)(F)F)cc1N1CCCC(F)(F)CC1]

In certain embodiments, the compound of the invention is selected from a group consisting of:

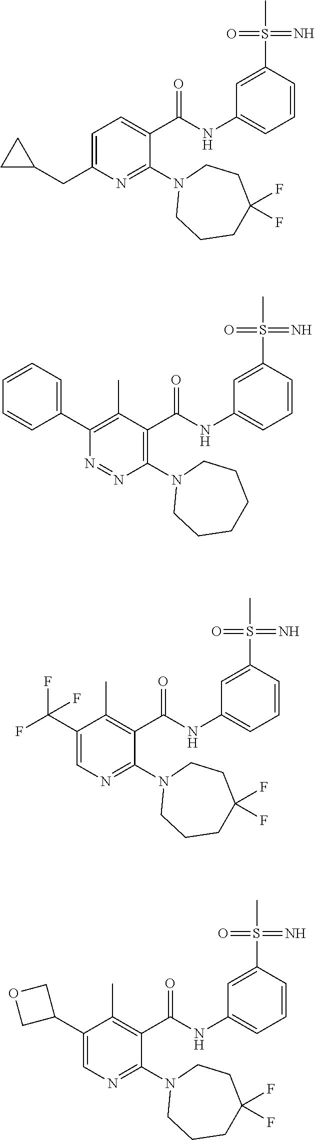 [SMILES: CS(=N)(=O)c1cccc(NC(=O)c2ccc(CC3CC3)nc2N2CCCC(F)(F)CC2)c1 ; Cc1c(-c2ccccc2)nnc(N2CCCCCC2)c1C(=O)Nc1cccc(S(C)(=N)=O)c1 ; Cc1c(C(F)(F)F)cnc(N2CCCC(F)(F)CC2)c1C(=O)Nc1cccc(S(C)(=N)=O)c1 ; Cc1c(C2COC2)cnc(N2CCCC(F)(F)CC2)c1C(=O)Nc1cccc(S(C)(=N)=O)c1]

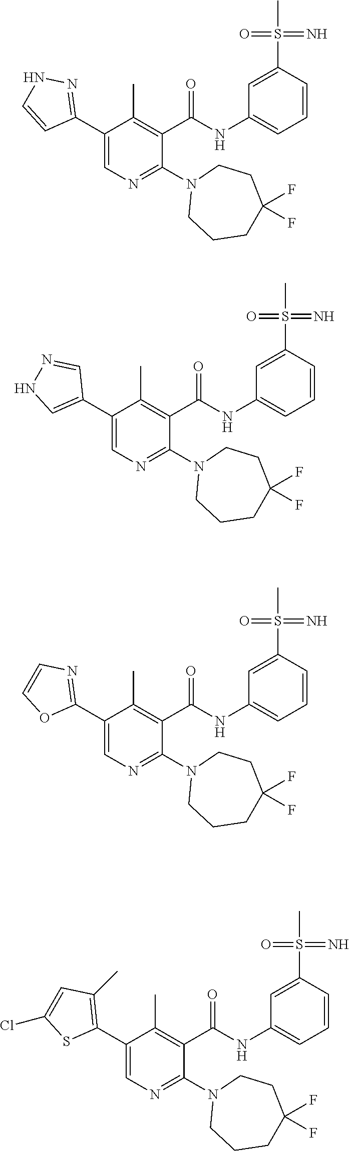 [SMILES: Cc1c(-c2cc[nH]n2)cnc(N2CCCC(F)(F)CC2)c1C(=O)Nc1cccc(S(C)(=N)=O)c1 ; Cc1c(-c2cn[nH]c2)cnc(N2CCCC(F)(F)CC2)c1C(=O)Nc1cccc(S(C)(=N)=O)c1 ; Cc1c(-c2ncco2)cnc(N2CCCC(F)(F)CC2)c1C(=O)Nc1cccc(S(C)(=N)=O)c1 ; Cc1cc(Cl)sc1-c1cnc(N2CCCC(F)(F)CC2)c(C(=O)Nc2cccc(S(C)(=N)=O)c2)c1C]

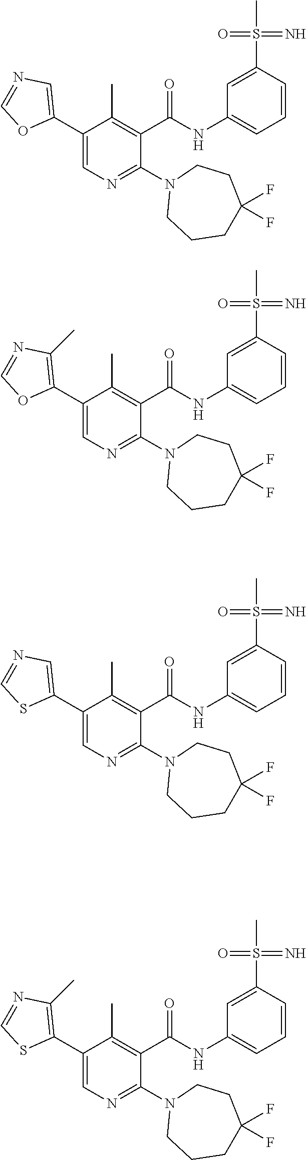 [SMILES: Cc1c(-c2cnco2)cnc(N2CCCC(F)(F)CC2)c1C(=O)Nc1cccc(S(C)(=N)=O)c1 ; Cc1ncoc1-c1cnc(N2CCCC(F)(F)CC2)c(C(=O)Nc2cccc(S(C)(=N)=O)c2)c1C ; Cc1c(-c2cncs2)cnc(N2CCCC(F)(F)CC2)c1C(=O)Nc1cccc(S(C)(=N)=O)c1 ; Cc1ncsc1-c1cnc(N2CCCC(F)(F)CC2)c(C(=O)Nc2cccc(S(C)(=N)=O)c2)c1C]

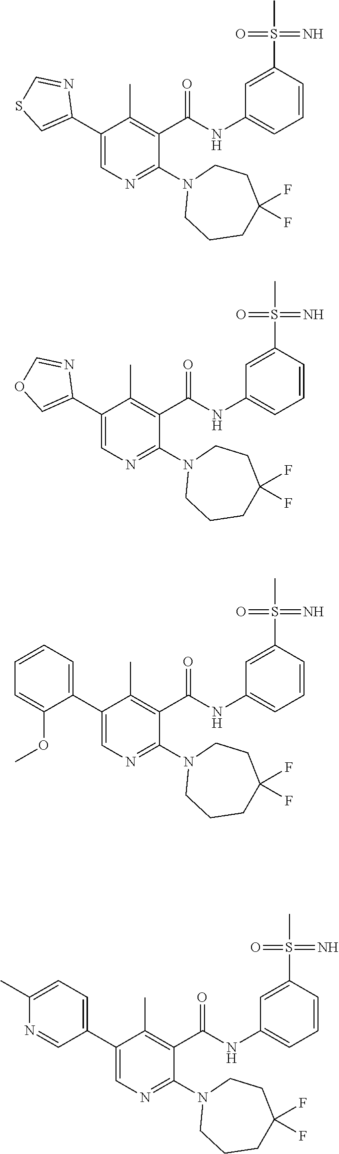 [SMILES: Cc1c(-c2cscn2)cnc(N2CCCC(F)(F)CC2)c1C(=O)Nc1cccc(S(C)(=N)=O)c1 ; Cc1c(-c2cocn2)cnc(N2CCCC(F)(F)CC2)c1C(=O)Nc1cccc(S(C)(=N)=O)c1 ; COc1ccccc1-c1cnc(N2CCCC(F)(F)CC2)c(C(=O)Nc2cccc(S(C)(=N)=O)c2)c1C ; Cc1ccc(-c2cnc(N3CCCC(F)(F)CC3)c(C(=O)Nc3cccc(S(C)(=N)=O)c3)c2C)cn1]

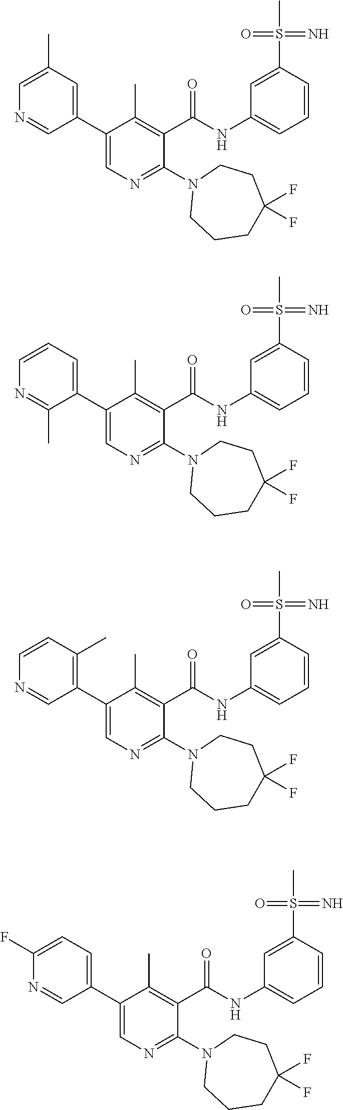 [SMILES: Cc1cncc(-c2cnc(N3CCCC(F)(F)CC3)c(C(=O)Nc3cccc(S(C)(=N)=O)c3)c2C)c1 ; Cc1ncccc1-c1cnc(N2CCCC(F)(F)CC2)c(C(=O)Nc2cccc(S(C)(=N)=O)c2)c1C ; Cc1ccncc1-c1cnc(N2CCCC(F)(F)CC2)c(C(=O)Nc2cccc(S(C)(=N)=O)c2)c1C ; Cc1c(-c2ccc(F)nc2)cnc(N2CCCC(F)(F)CC2)c1C(=O)Nc1cccc(S(C)(=N)=O)c1]

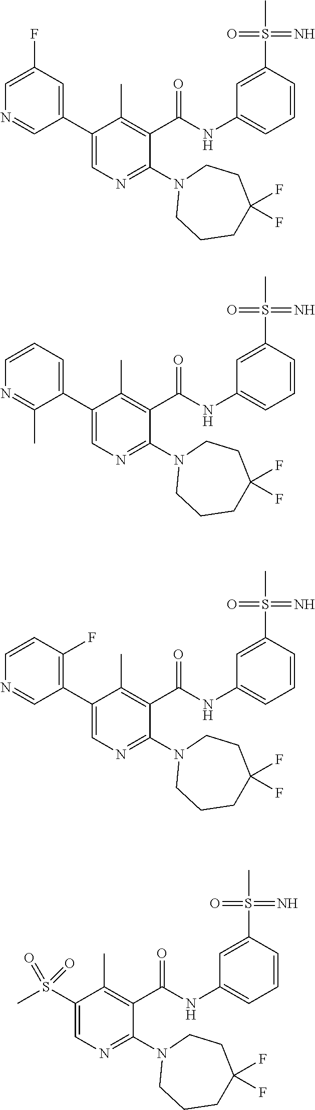 [SMILES: Cc1c(-c2cncc(F)c2)cnc(N2CCCC(F)(F)CC2)c1C(=O)Nc1cccc(S(C)(=N)=O)c1 ; Cc1ncccc1-c1cnc(N2CCCC(F)(F)CC2)c(C(=O)Nc2cccc(S(C)(=N)=O)c2)c1C ; Cc1c(-c2cnccc2F)cnc(N2CCCC(F)(F)CC2)c1C(=O)Nc1cccc(S(C)(=N)=O)c1 ; Cc1c(S(C)(=O)=O)cnc(N2CCCC(F)(F)CC2)c1C(=O)Nc1cccc(S(C)(=N)=O)c1]

In another aspect, the invention provides inhibitors of a voltage gated sodium channel Nav1.8. The inhibitors may have a defined chemical structure, such as the structure of any of the compounds described above.

In another aspect, the invention provides methods of treating a condition in a subject by providing to a subject having a condition a compound of the invention, such as any of those described above.

The condition may be associated with aberrant activity of voltage gated sodium channels.

The condition may be abdominal cancer pain, acute cough, acute idiopathic transverse myelitis, acute itch, acute pain, acute pain in major trauma/injury, airways hyperreactivity, allergic dermatitis, allergies, ankylosing spondylitis, asthma, atopy, Behcets disease, bladder pain syndrome, bone cancer pain, brachial plexus injury, burn injury, burning mouth syndrome, calcium pyrophosphate deposition disease, cervicogenic headache, Charcot neuropathic osteoarthropathy, chemotherapy-induced oral mucositis, chemotherapy-induced peripheral neuropathy, cholestasis, chronic cough, chronic itch, chronic low back pain, chronic pain, chronic pancreatitis, chronic post-traumatic headache, chronic widespread pain, cluster headache, complex regional pain syndrome, complex regional pain syndromes, constant unilateral facial pain with additional attacks, contact dermatitis, cough, dental pain, diabetic neuropathy, diabetic peripheral neuropathy, diffuse idiopathic skeletal hyperostosis, disc degeneration pain, distal sensory polyneuropathy (DSP) associated with highly active antiretroviral therapy (HAART), Ehlers-Danlos syndrome, endometriosis, epidermolysis bullosa, epilepsy, erythromelalgia, Fabry [symbol] disease, facet joint syndrome, failed back surgery syndrome, familial hemiplegic migraine, fibromyalgia, glossopharyngeal neuralgia, glossopharyngeal neuropathic pain, gout, head and neck cancer pain, inflammatory bowel disease, inflammatory pain, inherited erythromelalgia, irritable bowel syndrome, irritable bowel syndrome, itch, juvenile idiopathic arthritis, mastocytosis, melorheostosis, migraine, multiple sclerosis, musculoskeletal damage, myofascial orofacial pain, neurodegeneration following ischemia, neurofibromatosis type II, neuropathic ocular pain, neuropathic pain, neuropathic pain, nociceptive pain, non-cardiac chest pain, optic neuritis, oral mucosal pain, orofacial pain, osteoarthritis, osteoarthritis, overactive bladder, pachyonychia congenita, pain, pain resulting from cancer, pain resulting from chemotherapy, pain resulting from diabetes, pain syndrome, painful joint arthroplasties, pancreatitis, Parkinson [symbol] disease, paroxysmal extreme pain disorder, pemphigus, perioperative pain, peripheral neuropathy, persistent idiopathic dentoalveolar pain, persistent idiopathic facial pain, phantom limb pain, phantom limb pain, polymyalgia rheumatica, postherpetic neuralgia, post-mastectomy pain syndrome, postoperative pain, post-stroke pain, post-surgical pain, post-thoracotomy pain syndrome, post-traumatic stress disorder, preoperative pain, pruritus, psoriasis, psoriatic arthritis, pudendal neuralgia, pyoderma gangrenosum, radiotherapy-induced peripheral neuropathy, Raynaud [symbol] disease, renal colic, renal colic, renal failure, rheumatoid arthritis, salivary gland pain, sarcoidosis, sciatica, scleroderma, sickle cell disease, small fiber neuropathy, spinal cord injury pain, spondylolisthesis, spontaneous pain, stump pain, subacute cough, temporomandibular joint disorders, tension-type headache, trigeminal neuralgia, vascular leg ulcers, vulvodynia, or whiplash associated disorder. In another aspect, the invention provides methods of making a medicament using a compound of the invention, such as any of those described above.

In another aspect the invention provides products comprising a compound of the invention, such as any of those described above, for treatment of a condition, such as any of those described above, in a subject.

### DETAILED DESCRIPTION

### Definitions

Unless otherwise defined, all technical and scientific terms used herein have the same meaning as commonly understood by one of ordinary skill in the art to which this presently described subject matter belongs. The definitions provided below are intended to supplement and illustrate, not preclude, the definitions that would be apparent to one of ordinary skill in the art upon review of the present disclosure.

Unless otherwise stated, the moieties described below are optionally substituted, i.e., they may be substituted at one or more positions. The terms substituted, whether preceded by the term “optionally” or not, and substituent, as used herein, refer to the ability to change one or more functional groups for another functional group or groups on a molecule, provided that the valency of all atoms is maintained. When more than one position in any given structure may be substituted with more than one substituent selected from a specified group, the substituent may be either the same or different at every position. The substituents also may be further substituted (e.g., an aryl group substituent may have another substituent off it, such as another aryl group, which is further substituted at one or more positions).

When the term “independently selected” is used, the substituents being referred to (e.g., R groups, such as groups R₁, R₂, and the like, or variables, such as “m” and “n”), can be identical or different. For example, both R₁ and R₂ can be substituted alkyls, or R₁ can be hydrogen and R₂ can be a substituted alkyl, and the like.

The terms “a,” “an,” or “a(n),” when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

A named “R” or group will generally have the structure that is recognized in the art as corresponding to a group having that name, unless specified otherwise herein. For the purposes of illustration, certain representative “R” groups as set forth above are defined below.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Unless otherwise explicitly defined, a “substituent group,” as used herein, includes a functional group selected from one or more of the following moieties, which are defined herein.

The term hydrocarbon, as used herein, refers to any chemical group comprising hydrogen and carbon. The hydrocarbon may be substituted or unsubstituted. As would be known to one skilled in the art, all valences must be satisfied in making any substitutions. The hydrocarbon may be unsaturated, saturated, branched, unbranched, cyclic, polycyclic, or heterocyclic. Illustrative hydrocarbons are further defined herein below and include, for example, methyl, ethyl, n-propyl, isopropyl, cyclopropyl, ally 1, vinyl, n-butyl, tert-butyl, ethynyl, cyclohexyl, and the like.

The term “alkyl” by itself or as part of another substituent, means, unless otherwise stated, a straight (i.e., unbranched) or branched chain, acyclic or cyclic saturated hydrocarbon group, or combination thereof, and can include di- and multivalent groups, having the number of carbon atoms designated (e.g., C₁-C₁₀ means one to ten carbons, including 1, 2, 3, 4, 5, 6, 7, 8, 9, and 10 carbons). In particular embodiments, the term “alkyl” refers to C1-20 inclusive, including 1, 2, 3, 4, 5, 6, 7, 8, 9, 10, 11, 12, 13, 14, 15, 16, 17, 18, 19, and 20 carbons, linear (i.e., “straight-chain”), branched, or cyclic saturated hydrocarbon radicals derived from a hydrocarbon moiety containing between one and twenty carbon atoms by removal of a single hydrogen atom.

Representative saturated hydrocarbon groups include, but are not limited to, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, n-octyl, n-decyl, n-undecyl, dodecyl, cyclohexyl, (cyclohexyl)methyl, cyclopropylmethyl, and homologues and isomers thereof.

“Branched” refers to an alkyl group in which a lower alkyl group, such as methyl, ethyl, or propyl, is attached to a linear alkyl chain. “Lower alkyl” refers to an alkyl group having 1 to about 8 carbon atoms (i.e., a C1-8 alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, cyano, and mercapto.

The term “heteroalkyl,” by itself or in combination with another term, means, unless otherwise stated, a stable straight or branched chain having from 1 to 20 carbon atoms or heteroatoms or a cyclic hydrocarbon group having from 3 to 15 carbon atoms or heteroatoms, or combinations thereof, consisting of at least one carbon atom and at least one heteroatom, such as O, N, P, Si or S, and wherein the nitrogen, phosphorus, and sulfur atoms may optionally be oxidized and the nitrogen heteroatom may optionally be quaternized. The heteroatom(s) 0, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two or three heteroatoms may be consecutive, such as, for example, —CH₂—NH—OCH₃ and —CH₂—O—Si(CH₃)₃.

As described above, heteroalkyl groups, as used herein, include those groups that are attached to the remainder of the molecule through a heteroatom, such as —C(O)NR′, —NR′R″, —OR′, —SR, —S(O)R, and/or —S(O₂)R′.

“Cycloalkyl” refers to a saturated monocyclic or multicyclic ring system of from about 3 to about 15 carbon atoms, e.g., 3, 4, 5, 6, 7, 8, 9, or 10 carbon atoms. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, unsubstituted alkyl, substituted alkyl, aryl, or substituted aryl, thus providing a heterocyclic group. Representative monocyclic cycloalkyl rings include cyclopentyl, cyclohexyl, and cycloheptyl. Examples of cycloalkyl include, but are not limited to, cyclopentyl, cyclohexyl, 1-cyclohexenyl, 3-cyeiohexenyl, cycloheptyl, and the like.

The term “cycloalkylalkyl,” as used herein, refers to a cycloalkyl group as defined above, which is attached to the parent molecular moiety through an alkylene moiety, also as defined above, e.g., a C1-20 alkylene moiety. Examples of cycloalkylalkyl groups include cyclopropylmethyl and cyclopentylethyl.

The term “carbocyclyl” refers to a monocyclic or multicyclic ring system of from about 3 to about 15 ring members in which all ring members are carbon atoms. Unless otherwise specified, a carbocyclyl may be saturated, partially saturated (i.e., have one or more double or triple bonds), or aromatic.

The term “heterocyclyl” refers to a monocyclic or multicyclic ring system of from about 3 to about 15 ring members in which at least one ring member is a heteroatom, such as N, O, or S.

Unless otherwise specified, a heterocyclyl may be saturated, partially saturated (i.e., have one or more double or triple bonds), or aromatic. Examples of saturated and partially unsaturated non-aromatic heterocyclic groups include, but are not limited to, 3-oxetanyl, 2-oxetanyl, azetidinyl, thietanyl, tetrahydrofuranyl, tetrahydrothiophenyl, pyrrolidinyl, dihydropyranyl, tetrahydropyranyl, thio-dihydropyranyl, thio-tetrahydropyranyl, piperidinyl, piperazinyl, morpholinyl, 1,3-oxazinanyl, 1,3-thiazinanyl, 4,5,6-tetrahydropyrimidinyl, 2,3-dihydrofuranyl, dihydrothienyl, dihydropyridinyl, tetrahydropyridinyl, isoxazolidinyl, pyrazolidinyl, tetrazolyl, imidazolyl, isothiozolyl, triazolyl, azabicyclo-octanyl, diazabicyclo-octanyl, and all alkyl, alkoxy, haloalkyl and haloalkoxy substituted derivatives of any of the aforementioned groups.

The terms “cycloheteroalkyl” and “heterocycloalkyl” refer to a saturated ring system, such as a 3- to 10-member cycloalkyl ring system, that include one or more heteroatoms. The heteroatoms may be the same or different and may be nitrogen (N), oxygen (O), or sulfur (S).

Examples of heterocycloalkyl include, but are not limited to, 1-(1,2,5,6-tetrahydropyridyi), 1-piperidmyl, 2-piperidinyl, 3-piperidinyl, 4-morpholinyl, 3-morpholinyl, tetrahydrofuran-3-yl, tetrahydrofuran-3-yl, tetrahydrothien-2-yl, tetrahydrothien-3-yl, 1-piperazinyl, 2-piperazinyl, and the like.

The cycloheteroalkyl ring can be optionally fused to or otherwise attached to other cycloheteroalkyl rings and/or non-aromatic hydrocarbon rings. Heterocyclic rings include those having from one to three heteroatoms, such as oxygen, sulfur, and nitrogen, in which the nitrogen and sulfur heteroatoms may optionally be oxidized, and the nitrogen heteroatom may optionally be quaternized. Examples include, but are not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Representative cycloheteroalkyl ring systems include, but are not limited to pyrrolidinyl, pyrrolinyl, imidazolidinyl, imidazolinyl, pyrazolidinyl, pyrazolinyl, piperidinyl, piperazinyl, indolinyl, quinuclidinyl, morpholinyl, thiomorpholinyl, thiadiazinanyl, tetrahydrofuranyl, and the like.

An unsaturated hydrocarbon, carbocyclyl, or heterocyclyl has one or more double bonds or triple bonds. Examples of unsaturated hydrocarbons include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

The term “alkenyl” as used herein refers to a monovalent group derived from a C₂-C₂₀ inclusive straight or branched hydrocarbon moiety having at least one carbon-carbon double bond by the removal of a single hydrogen molecule. Alkenyl groups include, for example, ethenyl (i.e., vinyl), propenyl, butenyl, 1-methyl-2-buten-1-yl, pentenyl, hexenyl, octenyl, allenyl, and butadienyl.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkynyl” as used herein refers to a monovalent group derived from a straight or branched C₂-C₂₀ hydrocarbon of a designed number of carbon atoms containing at least one carbon-carbon triple bond. Examples of “alkynyl” include ethynyl, 2-propynyl (propargyl), 1-propynyl, pentynyl, hexynyl, and heptynyl groups, and the like.

The term “alkylene” by itself or a part of another substituent refers to a straight or branched bivalent aliphatic hydrocarbon group derived from an alkyl group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched, or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (CH₂)₃, cyclohexylene (—C₆H₁₀—, —CH═CH—CH═CH—, —CH═CH—CH₂—, —CH₂CH₂CH₂CH₂CH₂—, —CH₂CH₂CH(CH₂CH₂CH₃)CH₂—, —(CH₂)q-N(R)—(CH₂)r-, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—).

The term “heteroalkylene” by itself or as part of another substituent means a divalent group derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms also can occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—.

The term “spirocyclyl” refers to a polycyclic compound in which two rings have a single atom, e.g., carbon, as the only common member of two rings. Thus, a “spirocycloalkyl” refers to a cycloalkyl group with two rings having a single carbon in common, and a “spiroheterocycloalkyl” or “spiroheterocycloalkyl” refers to a cycloheteroalkyl group with two rings having a single carbon or other atom, e.g., nitrogen, in common.

The term “aryl” means, unless otherwise stated, an aromatic hydrocarbon substituent that can be a single ring or multiple rings (such as from 1 to 3 rings), which are fused together or linked covalently.

The term “heteroaryl” refers to and groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyndyl, 4-pyridyl, 2-pyrimidyl, 4-pyrimidyl, 5-benzoihiazolyl, purinyl, 2-benzimidazolyl, 5-indolyl, 1-isoquinolyl, 5-isoquinolyl, 2-quinoxalinyl, 5-quinoxalinyl, 3-qumolyl, and 6-quinolyl.

Substituents for each of above noted aryl and heteroaryl ring systems are selected from the group of acceptable substituents described below. The terms “arylene” and “heteroarylene” refer to the divalent forms of aryl and heteroaryl, respectively.

Where a heteroalkyl, heterocycloalkyl, or heteroaryl includes a specific number of members (e.g., “3 to 7 membered”), the term “member” refers to a carbon atom or heteroatom.

Each of the above terms is meant to include both substituted and unsubstituted forms of the indicated group. In some instances, the groups are explicitly defined as substituted, for example, “substituted aryl.” The optional substituents are provided below.

Substituents can be one or more of a variety of groups selected from, but not limited to: —OR′, ═O, ═NR′, ═N—OR′, —NR′R″—SR′, -halogen, —SiR′R″R″, —OC(O)R, —C(O)R, —CO₂R—C(O)NR′R″, —OC(O)NR′R″, —NR″C(O)R, —NR′—C(O)NR″R′, —NR″C(O)OR′, —NR—C(NR′R″)═NR″′, —S(O)R, —S(O)₂R′, —S(O)₂NR′R″, —NRSO₂R′, —CN, CF₃, fluorinated C₁-C₄ alkyl, and —NO₂ in a number ranging from zero to (2m′+1), where m′ is the total number of carbon atoms in such groups. R′, R″, R″′ and R″″ each may independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. Other non-limiting examples of substituents include (C₁-C₆)alkyl, (C₂-C₅)alkenyl, (C₃-C₅)alkynyl, halogen, halo(C₁-C₆)alkyl, hydroxy, —O(C₁-C₆)alkyl, halo(C₁-C₆)alkoxy, (C₃-C₅)cycloalkyl, (C₆-C₁₀)aryl, heterocyclyl, heteroaryl, amino, cyano, nitro, (C₁-C₆)alkyl-OH, (C₁-C₆)alkyl-O—(C₁-C₆)alkyl, (C₁-C₆)alkyl(C₆-C₁₀)aryl, —C(O)(C₁-C₆)alkyl, —C(O)NR′R″, —S(O)(C₁-C₆)alkyl, —S(O)NR′R″, —S(O)₂(C₁-C₆)alkyl, —S(O)₂NR′R″, —O(C₁-C₆)alkyl-S(O)(C₁-C₆)alkyl, —O(C₁-C₆)alkyl-S(O)NR′R″, —O(C₁-C₆)alkyl-S(O)₂(C₁-C₆)alkyl, and —O(C₁-C₆)alkyl-S(0)₂NR′R″. As used herein, an “alkoxy” group is an alkyl attached to the remainder of the molecule through a divalent oxygen.

When a compound of the disclosure includes more than one R group, for example, each of the R groups is independently selected as are each R′, R″, R″′ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of ordinary skill in the art will understand that the term “alkyl” is meant to include groups including carbon atoms bound to groups other than hydrogen groups, such as haloalkyl (e. g., —CF₃ and —CH₂CF₃) and acyl (e.g., —C(O)CH₃, —C(O)CF₃, —C(O)CH₂CH₃, and the like).

Two of the substituents on adjacent atoms of aryl or heteroaryl ring may optionally form a ring of the formula -T-C(O)—(CRR′)_{q}—U—, wherein T and U are independently —NR—, —O—, —CRR′— or a single bond, and q is an integer from 0 to 3. Alternatively, two of the substituents on adjacent atoms of aryl or heteroaryl ring may optionally be replaced with a substituent of the formula -A-(CH₂)r-B—, wherein A and B are independently —CRR′—, —O—, —NR—, —S—, —S(O)—, —S(O)₂—, —S(O)₂NR′— or a single bond, and r is an integer of from 1 to 4.

One of the single bonds of the new ring so formed may optionally be replaced with a double bond. Alternatively, two of the substituents on adjacent atoms of aryl or heteroaryl ring may optionally be replaced with a substituent of the formula —(CRR′)s-X′—(C″R″′)d-, where s and d are independently integers of from 0 to 3, and X′ is —O—, —NR′—, —S—, —S(O)—, —S(O)₂—, or —S(O)₂NR′—. The substituents R, R′, R″ and R″ may be independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “acyl” refers to an organic acid group wherein the —OH of the carboxyl group has been replaced with another substituent and has the general formula RC(═O)—, wherein R is an alkyl, alkenyl, alkynyl, aryl, carbocyclic, heterocyclic, or aromatic heterocyclic group as defined herein). As such, the term “acyl” specifically includes aryl acyl groups, such as a 2-(furan-2-yl)acetyl)- and a 2-phenylacetyl group. Specific examples of acyl groups include acetyl and benzoyl. Acyl groups also are intended to include amides, —RC(═O)NR, esters, —RC(═O)OR′, ketones, —RC(═O)R′, and aldehydes, —RC(═O)H.

The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₁-C₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, tert-butoxyl, and n-pentoxyl, neopentoxyl, n-hexoxyl, and the like.

The term “alkoxy alkyl” as used herein refers to an alkyl-O-alkyl ether, for example, a methoxy ethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described and includes substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplar) aralkyloxyl group is benzyloxyl, i.e., C₆H₅CH₂—O—. An aralkyloxyl group can optionally be substituted.

“Alkoxycarbonyl” refers to an alkyl-O—C(═O)— group. Exemplary alkoxy carbonyl groups include methoxycarbonyl, ethoxy carbonyl, butyloxycarbonyl, and tert-butyloxycarbonyl.

“Aryloxycarbonyl” refers to an aryl-O—C(═O)— group. Exemplary aryloxy carbonyl groups include phenoxy- and naphthoxy-carbonyl.

“Aralkoxycarbonyl” refers to an aralkyl —O—C(═O)— group. An exemplary aralkoxycarbonyl group is benzyloxycarbonyl.

“Carbamoyl” refers to an amide group of the formula —C(═O)NH₂.

“Alkylcarbamoyl” refers to a R′RN—C(═O) group wherein one of R and R′ is hydrogen and the other of R and R′ is alkyl and/or substituted alkyl as previously described. “Dialkylcarbamoyl” refers to a R′RN—C(═O)— group wherein each of R and R′ is independently alkyl and/or substituted alkyl as previously described.

The term “carbonyldioxyl,” as used herein, refers to a carbonate group of the formula —OC(═O)—OR.

“Acyloxyl” refers to an acyl-O— group wherein acyl is as previously described.

The term “amino” refers to the —NH₂ group and refers to a nitrogen containing group as is known in the art derived from ammonia by the replacement of one or more hydrogen radicals by organic groups. For example, the terms “acyl amino” and “alkylamino” refer to specific N-substituted organic groups with acyl and alkyl substituent groups respectively.

An “aminoalkyl” as used herein refers to an amino group covalently bound to an alkylene linker. More particularly, the terms alkylamino, dialkylamino, and trialkylamino as used herein refer to one, two, or three, respectively, alkyl groups, as previously defined, attached to the parent molecular moiety through a nitrogen atom. The term alkylamino refers to a group having the structure —NHR′ wherein R′ is an alkyl group, as previously defined; whereas the term dialkylamino refers to a group having the structure —NR′R″, wherein R′ and R″ are each independently selected from the group consisting of alkyl groups. The term trialkylamino refers to a group having the structure —NR′R″R″′, wherein R′, R″, and R″′ are each independently selected from the group consisting of alkyl groups. Additionally, R′, R″, and/or R″′ taken together may optionally be —(CH₂)k where k is an integer from 2 to 6. Examples include, but are not limited to, methylamino, dimethylamino, ethylamino, diethylamino, diethylaminocarbonyl, methylethylamino, isopropyl amino, piperidino, trimethylamino, and propylamine.

The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms alkylthioether and thioalkoxyl refer to a saturated (i.e., alkyl-S—) or unsaturated (i.e., alkenyl-S— and alkynyl-S—) group attached to the parent molecular moiety through a sulfur atom. Examples of thioalkoxyl moieties include, but are not limited to, methylthio, ethylthio, propylthio, isopropylthio, n-butylthio, and the like.

“Acylamino” refers to an acyl-NH— group wherein acyl is as previously described. “Aroylamino” refers to an aroyl-NH— group wherein aroyl is as previously described.

The term “carbonyl” refers to the —C(═O)— group, and can include an aldehyde group represented by the general formula R—C(═O)H.

The term “carboxyl” refers to the COOH group. Such groups also are referred to herein as a “carboxylic acid” moiety.

The term “cyano” refers to the —CN group.

The terms “halo,” “halide,” and “halogen” refer to fluoro, chloro, bromo, and iodo groups.

The term “haloalkyl” refers to an alkyl group substituted with one or more halogens.

Additionally, the term “haloalkyl,” includes monohaloalkyl and polyhaloalkyl. For example, the term “halo(C1-4)alkyl” includes, but is not limited to, trifluoromethyl, 2,2,2-trifluoroethyl, 4-chlorobutyl, 3-bromopropyl, and the like.

The terms “halocycloalky” and “cyclohaloalkyl” refer to a cycloalkly group with one or more halogens.

The term “hydroxyl” refers to the —OH group.

The term “hydroxy alkyl” refers to an alkyl group substituted with an —OH group.

The term “mercapto” refers to the —SH group.

The term “oxo” refers to an oxygen atom that is double bonded to a carbon atom or to another element.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term thiohydroxyl or thiol, as used herein, refers to a group of the formula —SH.

More particularly, the term “sulfide” refers to compound having a group of the formula —SR.

The term “sulfone” refers to compound having a sulfonyl group —S(O₂)R′.

The term “sulfoxide” refers to a compound having a sulfinyl group —S(O)R

The term ureido refers to a urea group of the formula —NH—CO—NH₂.

Throughout the specification and claims, a given chemical formula or name shall encompass all tautomers, congeners, and optical- and stereoisomers, as well as racemic mixtures where such isomers and mixtures exist.

Certain compounds of the present disclosure may possess asymmetric carbon atoms (optical or chiral centers) or double bonds; the enantiomers, racemates, diastereomers, tautomers, geometric isomers, stereoisomeric forms that may be defined, m terms of absolute stereochemistry, as (R)- or (S)- or, as D- or L- for amino acids, and individual isomers are encompassed within the scope of the present disclosure. The compounds of the present disclosure do not include those which are known in art to be too unstable to synthesize and/or isolate. The present disclosure is meant to include compounds in racemic, scalemic, and optically pure forms. Optically active (R)- and (S)-, or D- and L-isomers may be prepared using chiral synthons or chiral reagents or resolved using conventional techniques. When the compounds described herein contain olefenic bonds or other centers of geometric asymmetry, and unless specified otherwise, it is intended that the compounds include both E and Z geometric isomers.

Unless otherwise stated, structures depicted herein are also meant to include all stereochemical forms of the structure, i.e., the R and S configurations for each asymmetric center. Therefore, single stereochemical isomers as well as enantiomeric and diastereomeric mixtures of the present compounds are within the scope of the disclosure.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium, and which are readily converted from one isomeric form to another.

Unless otherwise stated, structures depicted herein are also meant to include compounds which differ only in the presence of one or more isotopically enriched atoms. For example, compounds having the present structures with the replacement of a hydrogen by a deuterium or tritium, or the replacement of a carbon by 13C- or 14C-enriched carbon are within the scope of this disclosure.

The compounds of the present disclosure may also contain unnatural proportions of atomic isotopes at one or more of atoms that constitute such compounds. For example, the compounds may be radiolabeled with radioactive isotopes, such as for example, tritium (3H), iodine-125 (¹²⁵I) or carbon-14 (¹⁴C). All isotopic variations of the compounds of the present disclosure, whether radioactive or not, are encompassed within the scope of the present disclosure.

The compounds of the present disclosure may exist as salts, and particularly as pharmaceutically acceptable salts. The present disclosure includes such salts. Examples of applicable salt forms include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures, succinates, benzoates, and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or m a suitable inert solvent or by ion exchange. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner.

The parent form of the compound differs from the various salt forms in certain physical properties, such as solubility in polar solvents.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

In addition to salt forms, the present disclosure provides compounds that are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

The term “protecting group” refers to chemical moieties that block some or all reactive moieties of a compound and prevent such moieties from participating in chemical reactions until the protective group is removed, for example, those moieties listed and described in T. W. Greene, P. G. M. Wuts, Protective Groups in Organic Synthesis, 3rd ed. John Wiley & Sons (1999). It may be advantageous, where different protecting groups are employed, that each (different) protective group be removable by a different means. Protective groups that are cleaved under totally disparate reaction conditions allow differential removal of such protecting groups.

For example, protective groups can be removed by acid, base, and hydrogenolysis. Groups such as trityl, dimethoxytrityl, acetal and tert-butyldimethylsilyl are acid labile and may be used to protect carboxy and hydroxy reactive moieties in the presence of amino groups protected with Cbz groups, which are removable by hydrogenolysis, and Fmoc groups, which are base labile.

Carboxylic acid and hydroxy reactive moieties may be blocked with base labile groups such as, without limitation, methyl, ethyl, and acetyl in the presence of amines blocked with acid labile groups such as tert-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be blocked with oxidatively-removable protective groups such as 2,4-dimethoxybenzyl, while co existing amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a palladium(O)-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

### Compounds

The invention provides compounds that modulate, e.g., inhibit, the activity of voltage gated sodium channels. In certain embodiments, the compound has the structure of Formula (I):

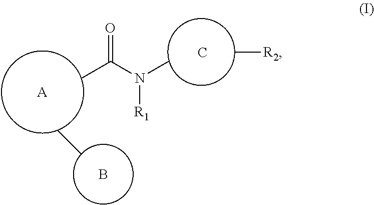

wherein:
   A is an aryl or heteroaryl ring containing one or more heteroatoms independently selected from O, S, and N, wherein A is unsubstituted or substituted with one or more substituents selected from a group consisting of:
   H, halo, C₁-C₆ alkyl, C₁-C₆ branched alkyl, C₁-C₆ alkenyl, C₁-C₆ alkynyl, C₁-C₆ haloalkyl, C₁-C₆ alkoxy, C₁-C₆ cycloalkoxy, C₁-C₆ haloalkoxy, nitro, cyano, —CH₂—(C₁-C₆)-cycloalkyl, —CF₂-cycloalkyl, —CH(CH₃)-cycloalkyl, —CH₂-aryl, —CF₂-aryl, —CH(—CH₃)-aryl, C(═O)—(C₁-C₆)-alkyl, —C(═O)-cycloalkyl, —C(═O)—NH-alkyl, —C(═O)NH₂, hydroxy, —COOH (and ester thereof), alkylsulfonyl, arylsulfonyl, sulfonamide, amino, —NR′R″, —NHSO₂R₁, —NHC(═O)-alkyl —NH(C═O)NR′R″, trifluoromethyl, bromo, chloro, fluoro, iodo, cyclopropylmethyl, sulfonylmethyl; 3-6-membered cycloalkyl; 3-6-membered heterocycloalkyl, 5-7 membered aryl, or 5-7 membered heteroaryl, any of which may have one or more substituents listed herein, wherein the 3-6 membered heterocycloalkyl or 5-7 membered heteroaryls comprise at least one heteroatom independently selected from O, S, and N;
   B is selected from the group consisting of a monocyclic, bicyclic, or spirocyclic cycloalkyl ring, and a monocyclic, bicyclic, or spirocyclic cycloheteroalkyl ring, wherein the cycloheteroalkyl contains 1-4 heteroatoms independently selected from N, O, and S, and wherein one or more carbons of the cycloalkyl or cycloheteroalkyl ring may be optionally substituted with one or more halogens or one or more haloalkyl groups;
   C is selected from the group consisting of aryl, heteroaryl, heterocyclyl, cycloalkyl, and bridged cycloalkyl, wherein said aryl, heteroaryl, heterocyclyl, and bridged cycloalkyl comprises one or more substitutions (in addition to the R₂ group) selected from a group consisting of: H, halo, alkyl, cyano, haloalkyl, and nitro;
   R₁ is selected from H and C₁-C₃ alkyl;
   R₂ is Formula (II):

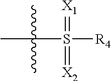

wherein:
X₁ is O and X₂ is NH or NR′;
X₁ is O and X₂ is NR₃; or
X₁ and X₂ are independently selected from NH or NR′;
wherein,
R₃ is independently selected from —(C═O)—(CH₂)ₙRₐ, —(C═O)—(CH₂)ₙ—(OCH₂CH₂)ₙRₐ, —(C═O)—(CH₂)ₙ—(OCH₂CH₂O)ₙRₐ, —(C═O)—(CH₂)ₙ—(NHCH₂CH₂)ₙRₐ, —(C═O)—(CH₂)ₙ—(NHCH₂CH₂O)ₙRₐ, —(CH₂)ₙRₐ, —(CH₂)ₙ(CR_{b}R_{c})Rₐ, —(C═O)—(NHCH₂CH₂NH)ₙRₐ, —(C═O)—(CH R_{b})Rₐ, —(CH₂)ₙ(CR_{b}R_{c}) (CH₂)ₙRₐ, or —(C═O)—(CR_{b}R_{c})Rₐ;
Rₐ is H, C₁-C₆-alkyl, branched alkyl, cycloakyl, aryl, heteroaryl, alkenyl, alkynyl, haloalkyl, alkoxy, cycloalkoxy, haloalkoxy, —CF₂-cycloalky, —CH(CH₃)-cycloalkyl, —CF₂-aryl, —NH₂, —NR′R″, amino cycloalkyl, 4 to 7 member heterocyclyl, —OH, —COONH₂, —COOH (and ester thereof), alkylsulfonyl, arylsulfonyl, sulfonamide, or amino;
R_{b} is selected from F, C₁-C₆-alkyl, haloalkyl, branched alkyl, aryl, heteroaryl, 3-7 membered carbocylyl, 4-7 membered heterocyclyl with one or more heteroatoms;
R_{c} is C₁-C₆-alkyl or F;
R_{c} R_{b} together with the carbon atom to which attached form a 3-6 membered carbocyclic ring or 4-6 membered heterocyclic ring with one or more hetero atoms;
R_{b} is selected from F, C₁-C₆-alkyl, haloalkyl, branched alkyl, aryl, heteroaryl, 3-7 membered carbocylyl, 4-7 membered heterocyclyl with one or more heteroatoms;
R_{c} is C₁-C₆-alkyl or F;
R_{c} R_{b} together with the carbon atom to which attached form a 3-6 membered carbocyclic ring or 4-6 membered heterocyclic ring with one or more hetero atoms;
R₄ is selected from C₁-C₃ alkyl, C₃-C₄ cycloalkyl, haloalkyl, halocycloalkyl, aryl, heteroaryl, or heterocyclyl; and
n=0, 1, 2, 3, 4, 5, 6.

In certain embodiments, R₁ is selected from H and C₁-C₃ alkyl

In certain embodiments, R₁ is H.

In certain embodiments, R₂ is H.

In various embodiments, if the A ring is a heteroaryl, it contains one or more nitrogen atom in a ring. In certain embodiments, the A ring contains one nitrogen atom. In certain embodiments, such nitrogen atom may be in the form of an N-oxide, wherein the N-oxide is selected from a group consisting of pyridyl N-oxide, pyrazinyl N-oxide, and pyrimidinyl N-oxide, pyridazinyl N-oxide. In certain embodiments, the A ring may include further substituents.

In various embodiments, ring C is optionally fused to at least one selected from the group consisting of: an optionally saturated carbocyclyl containing 5-6 ring members or an optionally saturated heterocyclyl containing 5-6 ring members and 1-4 optionally charged heteroatoms.

In certain embodiments, R₂ is selected from H or formula (II):

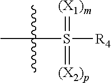

wherein:
m and p are either 0 or 1; and
X₁ is O and X₂ is either NH or NR′; or
X₁ is O and X₂ is NR₃
R₃ is defined above.

In various embodiments, R₂ is selected from a group consisting of: H, —C(═O)NH₂, —C(═O)NHR′, —C(═O)NR′R″, —C(═O)OH, alkylamino, and —S(═O)R′.

In various embodiments, R₂ is selected from H or Formula (II):

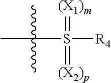

wherein:
m and p are either 0 or 1; and
X₁ is O, NH, NR′ and X₂ is either NH or NR′, and R₄ is described above.

In certain embodiments, R₂ is represented by Formula (II):

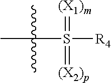

wherein:
m and p are either 0 or 1;
X₁ and X₂ are both O or NH; and R₄ is described above.

In certain embodiments, R₂ is represented by Formula (II) (described above), wherein X₁ is not O and/or X₂ is not O.

In certain embodiments, R₂ is represented by Formula (II) (described above), wherein X₁ is not NH or NR′ and X₂ is not NH or NR′. In certain embodiments, ring A is an optionally substituted aryl.

In certain embodiments, ring A is an optionally substituted heteroaryl with one heteroatom.

In certain embodiments, ring A is an optionally substituted heteroaryl with two heteroatoms.

In certain embodiments, ring A is substituted with at least triflouoromethyl. In certain embodiments, trifluoromethyl is the only substitution on ring A.

In certain embodiments, ring A is substituted with at least cyclopropylmethyl.

In certain embodiments, A is represented by the following Formula(s):

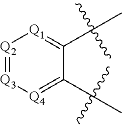

wherein each of Q₁, Q₂, Q₃ and Q₄ is independently N, N—O, or CR₅;
wherein R₅ is H, hydroxyl, halogen, —CD₃, alkyl, haloalkyl, alkoxy, haloalkoxy, alkyl sulfonyl, alkyl sulfoximinyl, alkyl sulfonamide, cyano, —CF₃, —OCF₃, heterocyclyl in which each ring has 4 to 6 members, heteroaryl having a 5 or 6 ring membered, saturated heterocyclyl, or partially unsaturated heterocyclyl, O-aryl in which each ring has 5 or 6 members, O-heteroaryl in which each ring has 5 or 6 members, O-cycloalkyl, O-cycloheteroalkyl, each of which is optionally substituted.

In certain embodiments, ring A is an optionally substituted five (5) or more membered saturated or partially unsaturated heterocyclic ring having one or two heteroatoms independently selected from N, O, or S.

In certain embodiments, ring A is represented by the following Formula(s):

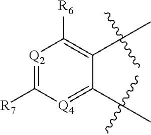

wherein:
Q₂ and Q₄ is N, N—O;
Q₂ is N, N—O; Q₄ is CR₅;
Q₂ is CR₅; Q₄ is N, N—O;
R₆ is H, halogen, —CD₃, C₁-C₆ alkyl, branched alkyl, alkenyl, alkynyl, haloalkyl, alkoxy, cycloalkyl, heterocyclyl, NH₂, NHR′, NR′R″, NHC(═O)R′, NHSO₂R, —C(═O)R′, —C(═O)NHR′, —C(═O)NR′R″, —O—R′, —O—C(═O)—R′, aryl, heteroaryl, —CF₂CH₃, —CF₂CF₃;
R₇ is H, hydroxyl group, halogen, —CD₃, C₁-C₆ alkyl, branched alkyl, allyl, alkenyl, alkynyl, haloalkyl, alkoxy, cycloalkoxy, haloalkoxy, nitro, cyano, —CH₂-cycloalkyl, —CF₂CH₃, —CF₂CF₃, CH₂CF₂, —CF₂-cycloalky, —CH(CH₃)-cycloalkyl, —CH₂-aryl, —CF₂-heteroaryl, —CF₂-heterocyclyl, —CH(—CH₃)-aryl, C(═O)-alkyl, —C(═O)cycloalkyl, —C(═O)—NH-alkyl, —C(═O)NH₂, —C(═O)NHR′, —C(═O)NR′R″, hydroxy, —COOH (and ester thereof), alkylsulfonyl, arylsulfonyl, sulfonamide, amino, NR′R″, —NHSO₂R′, —NHC(═O)-alkyl —NH(C═O)NR′R″, trifluoromethyl, cyclopropylmethyl, methylsulfonyl, 3-6 membered cycloalkyl; 3-6 membered heterocycloalkyl, any of which may have one or more substituents, wherein the 3-6 membered heterocycloalkyl comprises at least one heteroatom independently selected from O, S, and N;
wherein not more than two of Q₁, Q₂, Q₃ and Q₄ are N or N—O.

In certain embodiments, ring A is represented by the following Formula(s):

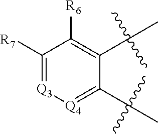

wherein:
Q₃ and Q₄ are N; wherein Q₃ is N, N—O; Q₄ is CR₅; or wherein Q₃ is CR₅, Q₄ is N, N—O;
R₅ is defined above; and
R₆ and R₇ are defined above.

In certain embodiments, ring A is represented by the following Formula(s):

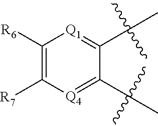

wherein Q₁ and Q₄ are N, N—O; Q₁ is N, N—O; Q₄ is CR₅; Q₁ is CR₅, Q₄ is N, N—O; or Q₁ and Q₄ are CR₅; and wherein R₆ and R₇ are defined above.

In certain embodiments, ring A is represented by the following Formula(s):

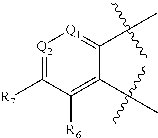

wherein:
   Q₁ and Q₂ are N; and
   R₆ and R₇ are defined above.

In certain embodiments, ring A is represented by the following Formula(s):

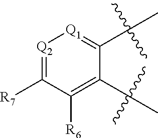

wherein:
   Q₁ is CR₅, and Q₂ is N;
   R₆ and R₇ are defined above.

In certain embodiments, ring A is represented by the following formula(s):

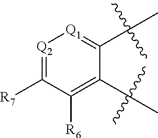

wherein Q₁ is N; and Q₂ is CR₅; and R₆ and R₇ are defined above.

In certain embodiments, ring A is selected from the group consisting of:

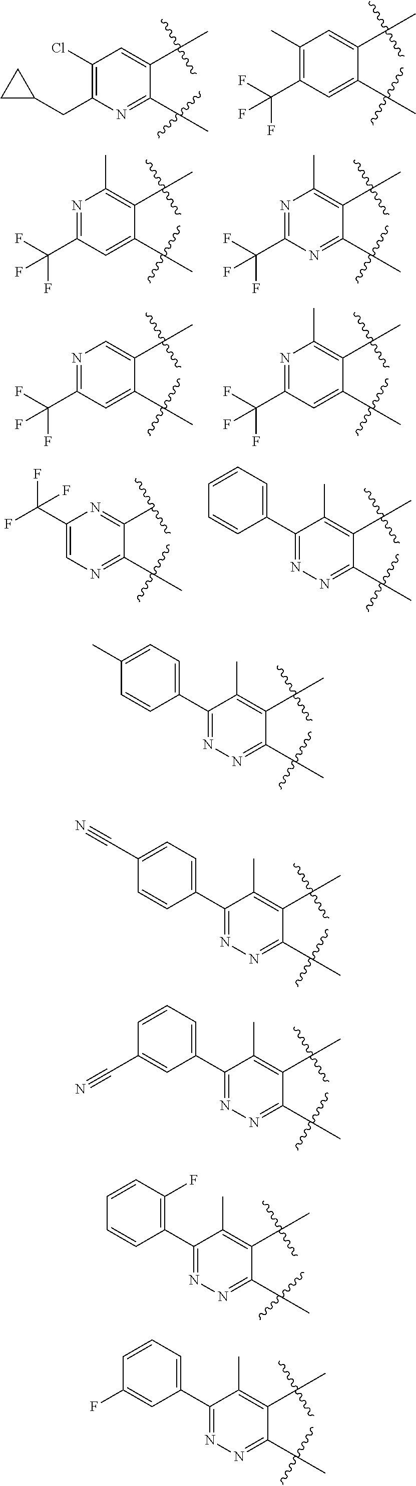

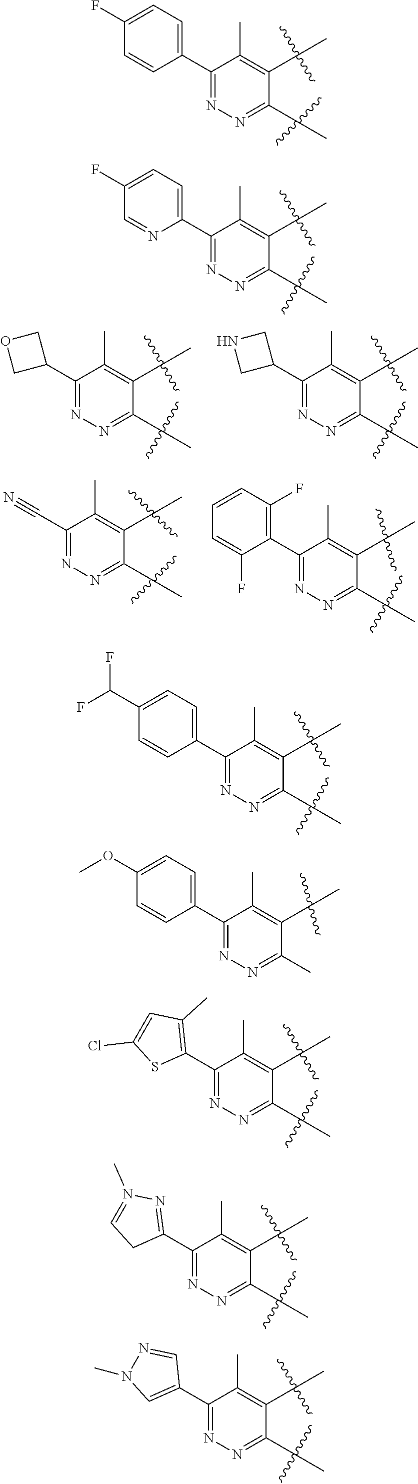

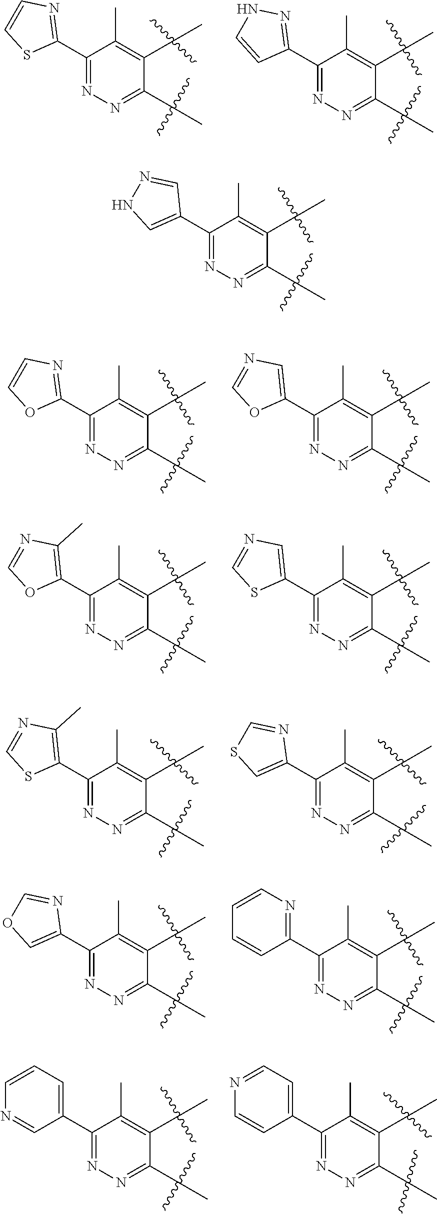

In certain embodiments, ring B is selected from the group consisting of pyrrolidine, azetidine, piperidine, piperazine, azepane, azocane, morpholine, thiomorpholine, oxazepane, isoindoline, dihydroisoquinoline, octahydroisoindole, azabicyclo[2.2.1]heptane, azabicyclo[3.1.1]heptane, azabicyclo[4.1.0]heptane, azabicyclo[3.2.1]octane, diazabicyclo-[3.2.1]octane, azabicyclo[3.2.0]heptane, oxa-azabicyclo[3.2.1]octane, azaspiro[2.5]octane, azaspiro[2.6]nonane, azaspiro[3.5]nonane, oxa-azaspiro[3.5]nonane, oxa-azaspiro[4.5]decane, dihydrothieno[3,2-c]pyridine, dihydrothiazolo[4,5-c]pyridine, dihydrooxazolo[4,5-c]pyridine, dihydroimidazo[1,2-a]pyrazine, hexahydrofuro [3,2-b]pyrrole, hexahydrocyclopenta[c]pyrrole, and azatricyclo[4.3.1.13,8]undecane.

In certain embodiments, ring C is phenyl.

In certain embodiments, ring C is pyridyl.

In certain embodiments, in Formula (II), X₁ and X₂ are O.

In certain embodiments, in Formula (II), X₁ is O and X₂ is NH.

In certain embodiments, the compound of Formula (I) is further described as Formula (III):

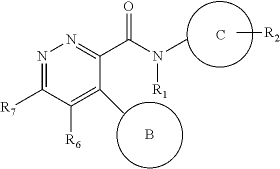

wherein:
   A, B, C, R₁, and R₂ are defined above, and R₆ and R₇ are defined above.

In certain embodiments, substituted or unsubstituted B is selected from a group consisting of:

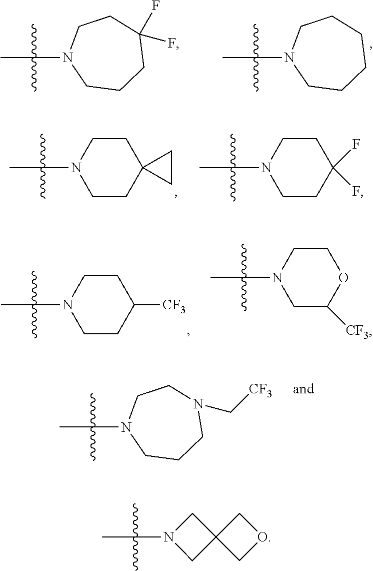

In certain embodiments, substituted or unsubstituted C is selected from a group consisting of:

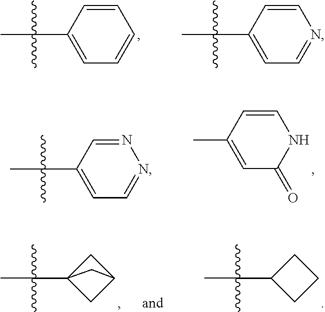

In certain embodiments, R₁ is H.

In certain embodiments, R₂ is selected from a group consisting of: H,

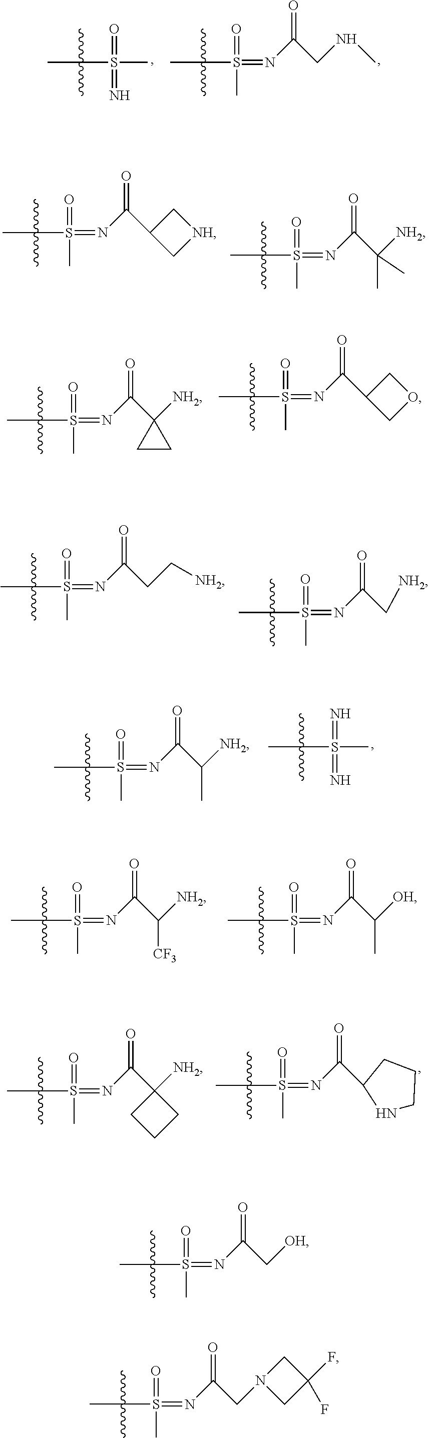

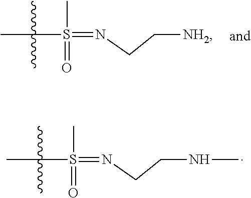

In certain embodiments, R₂ is selected from a group consisting of H,

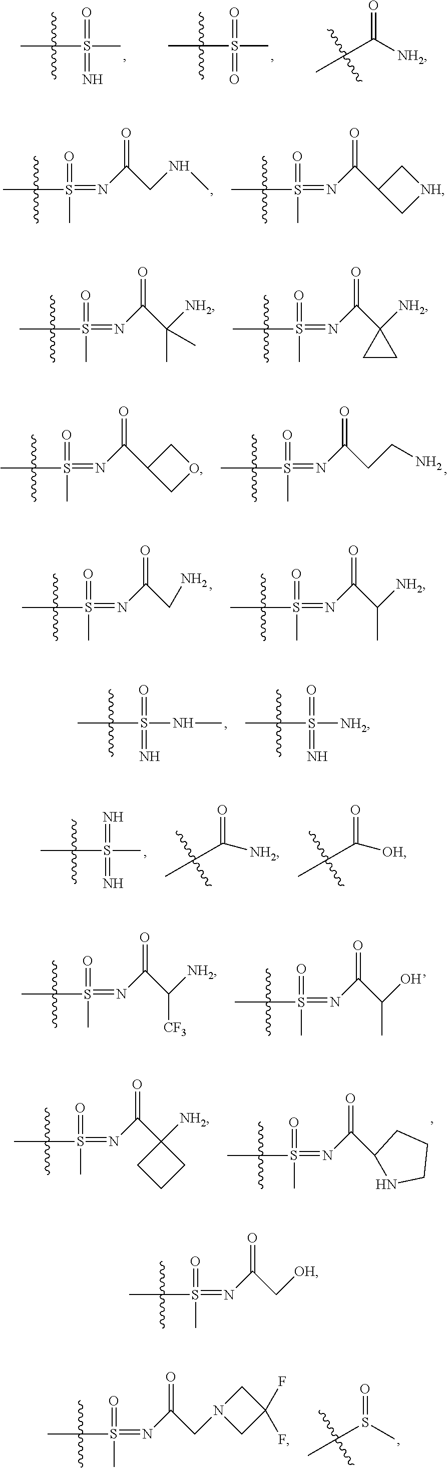

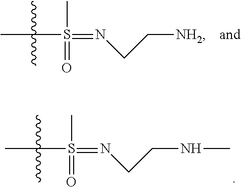

In certain embodiments, R₂ is:

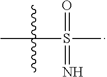 [SMILES: *S(C)(=N)=O]

In certain embodiments, ring C comprises additional substitutions, i.e., substitutions in wherein the substitutions are selected from a group consisting of H, halo, and alkyl. In certain embodiments, the said halo substitution in ring C is F.

In certain embodiments, the one or more substitutions on the ring A ring are selected from a group consisting of: halo, cyano, haloalkyl, cyanoalkyl, substituted or unsubstituted C₁-C₆ alkyl, aryl, C₃-C₆ cycloalkyl, C₃-C₆ heterocycloalkyl, C₃-C₆ heteroaryl, and a combination thereof, wherein the heterocycloalkyl and heteroaryl comprise one or more hetero atoms selected from a group consisting of: N, O, or S.

In certain embodiments, the one or more substitutions on the ring A are selected from a group consisting of: methyl, trifluoromethyl, chloro, fluoro, bromo, C₁-C₆ alkyl, phenyl, cycloalkyl, methyl pyrazole, fused 1,4 dioxane, and methylcyano.

In certain embodiments, the one or more substitutions on the ring A are selected from a group consisting of: —CH₃, —CF₃, —Cl, —Br, —F, —CH₂—CH₂—CH═CH₂, phenyl, —CH₂—CN, —C(═O)—NH₂,

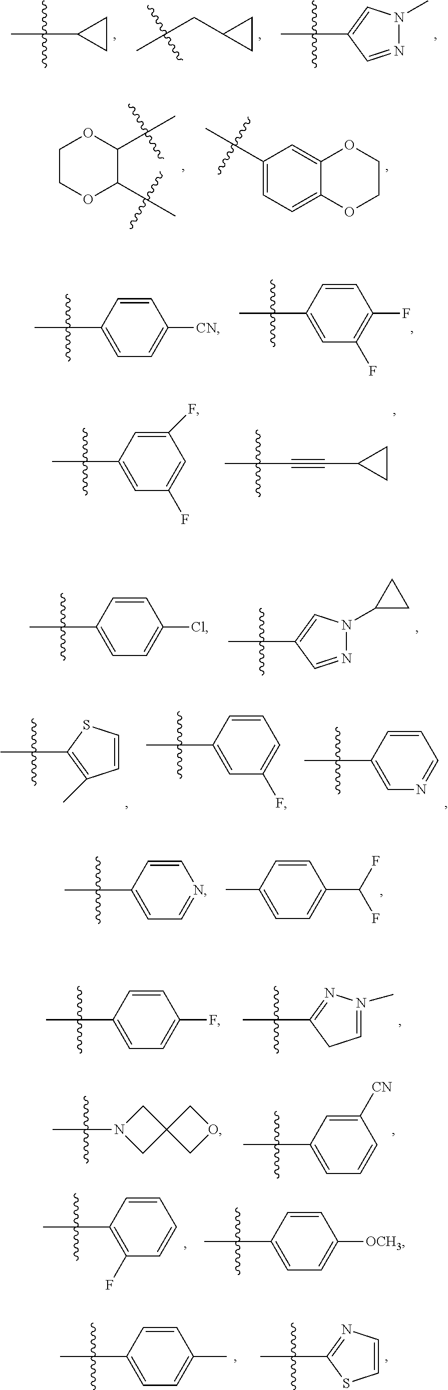

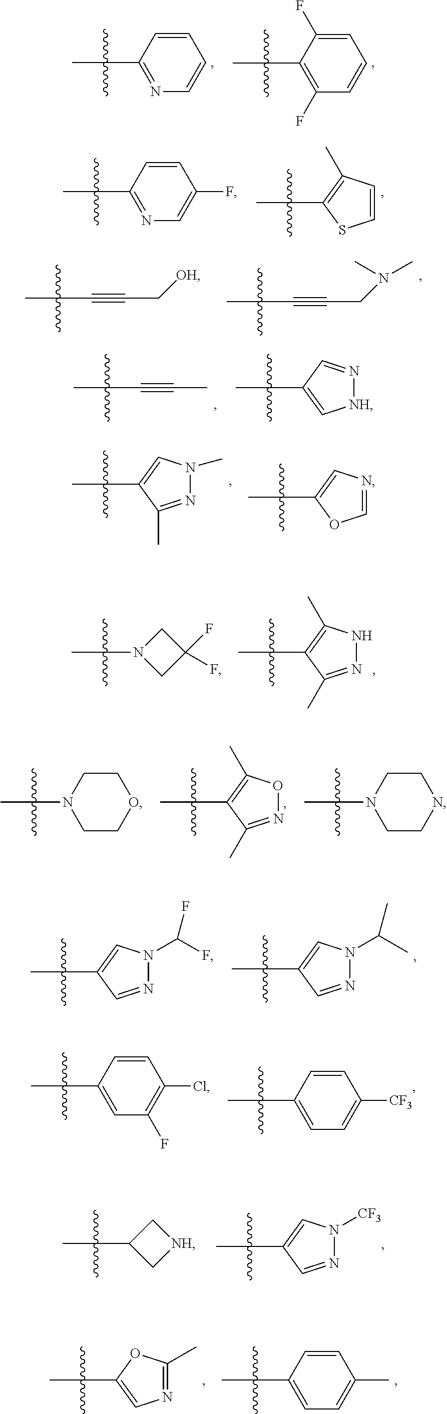

and any combination thereof.

In another aspect, the invention provides inhibitors of a voltage gated sodium channel Nav1.8. The inhibitors may have a defined chemical structure, such as the structure of any of the compounds described above.

In another aspect, the invention provides methods of treating a condition in a subject by providing to a subject having a condition a compound of the invention, such as any of those described above.

In another aspect the invention provides products comprising a compound of the invention, such as any of those described above, for treatment of a condition, such as any of those described above, in a subject.

In certain embodiments, the compound of Formula (I) is selected from the group consisting of:

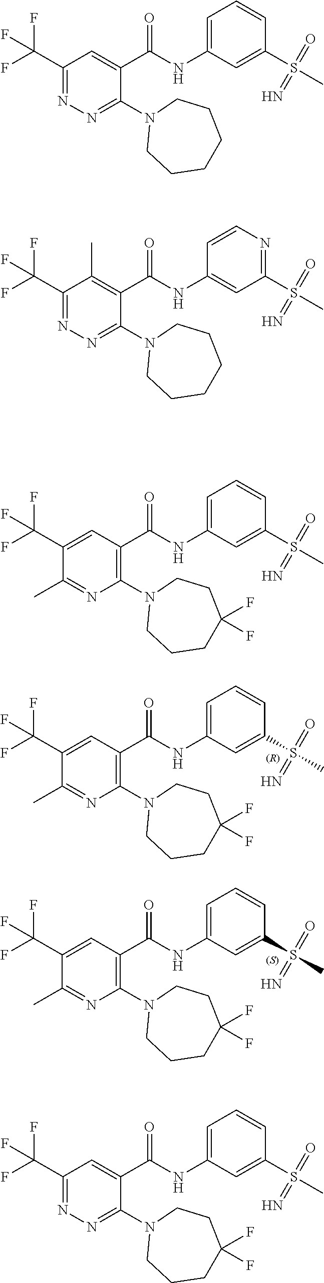

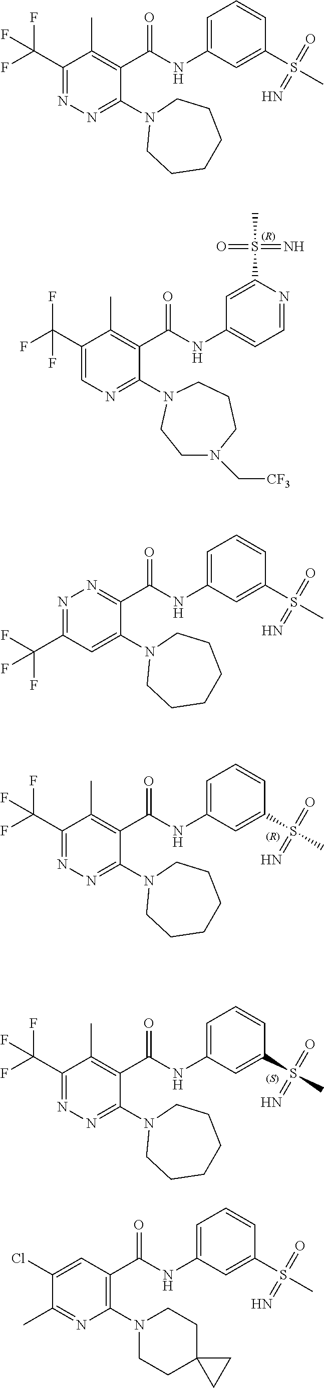

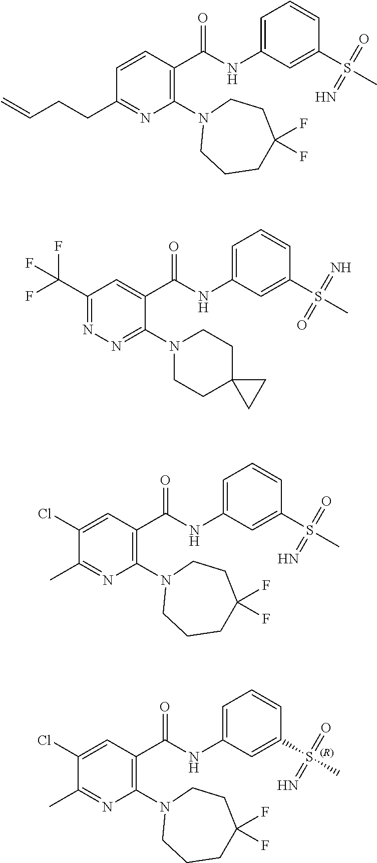

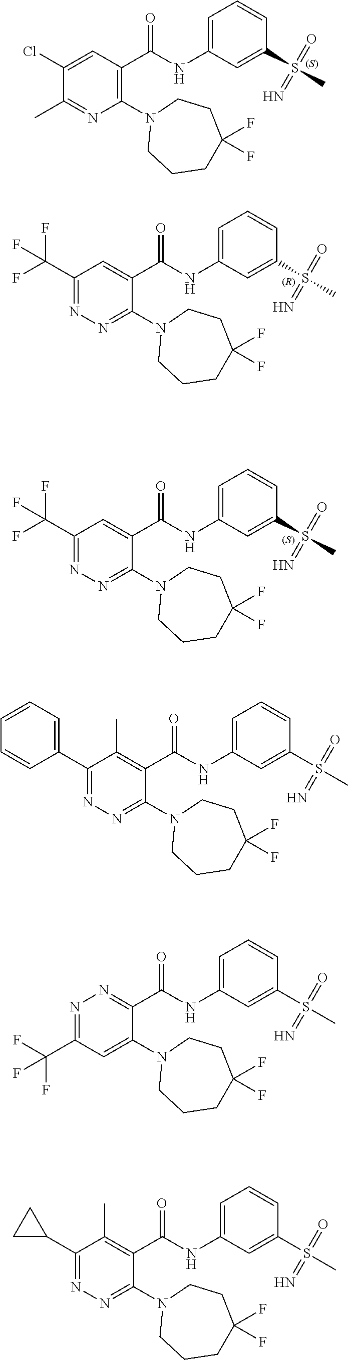

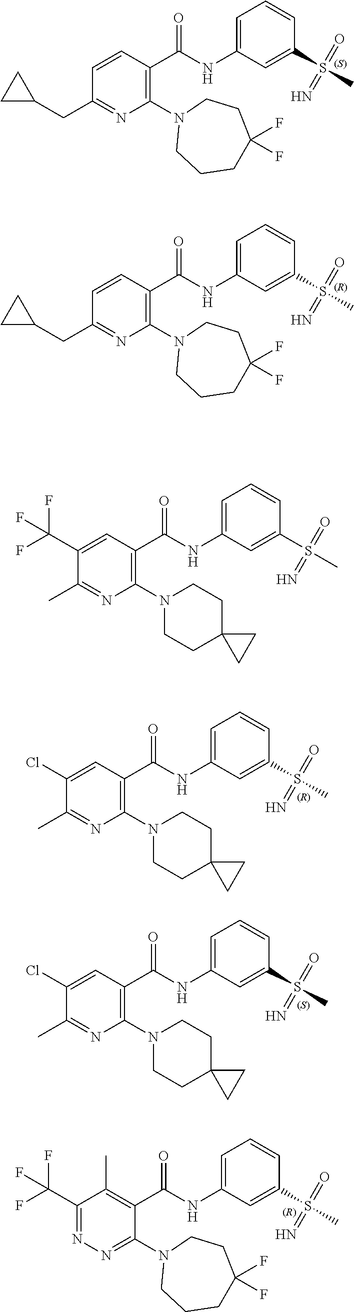

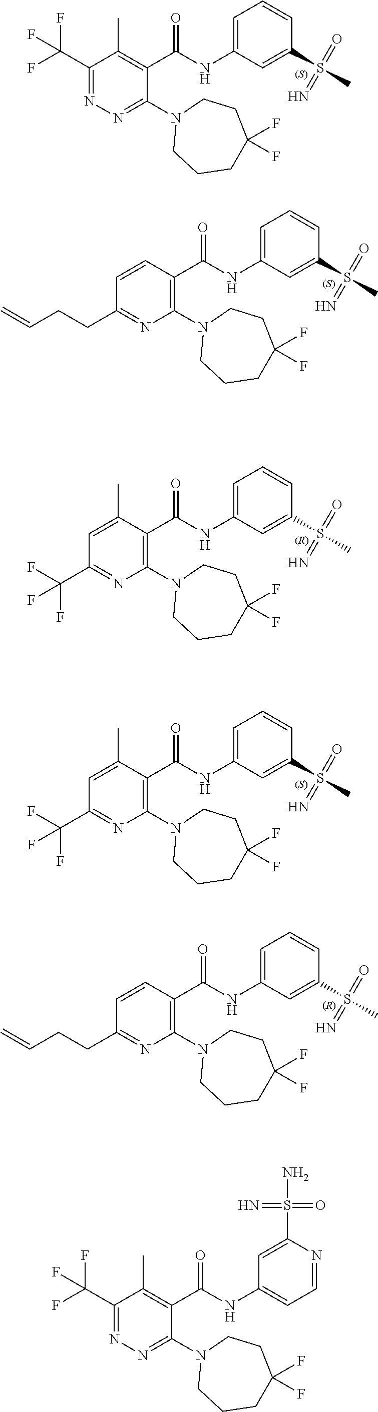

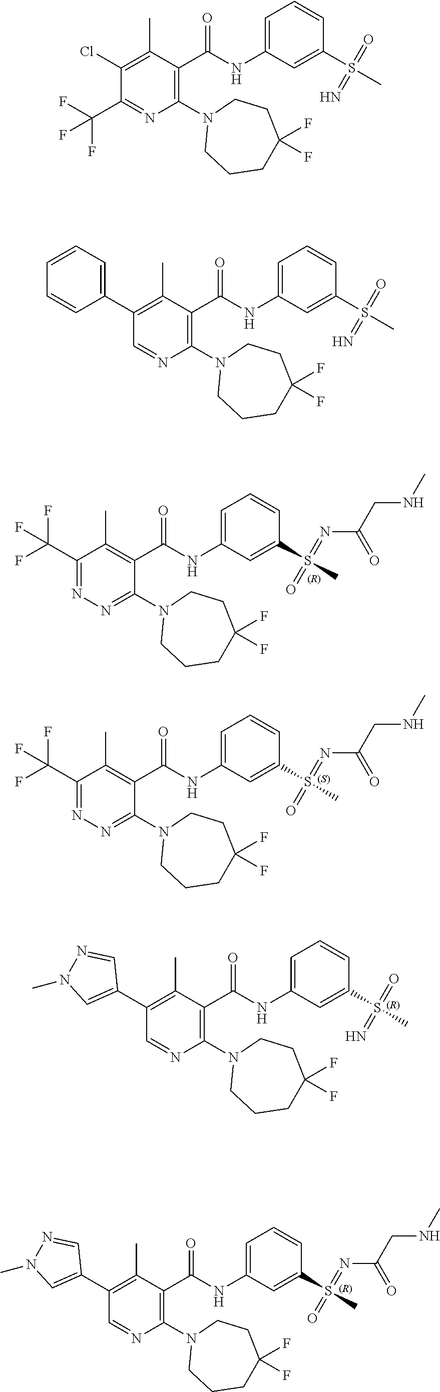

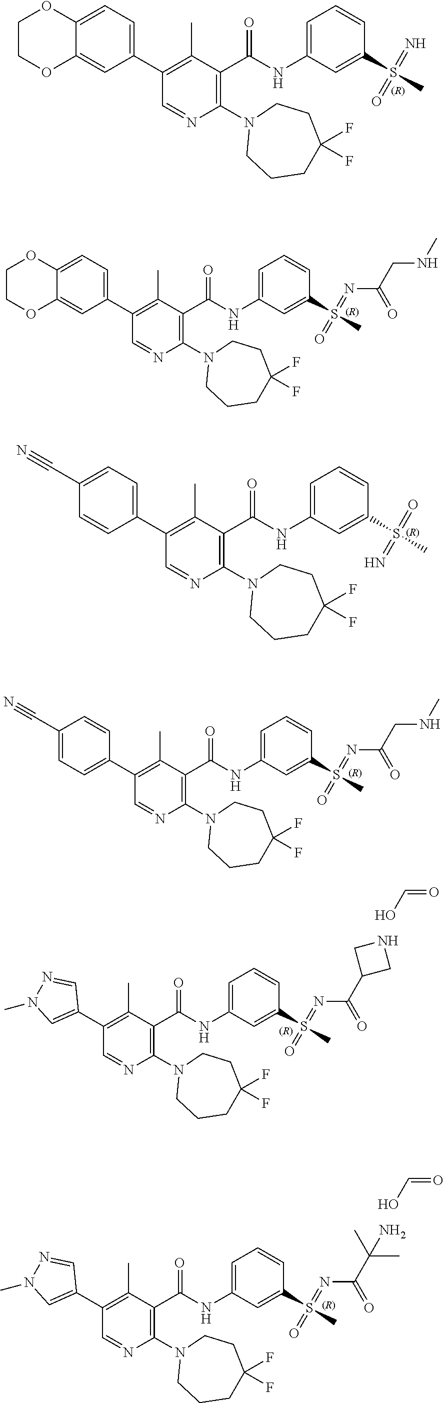

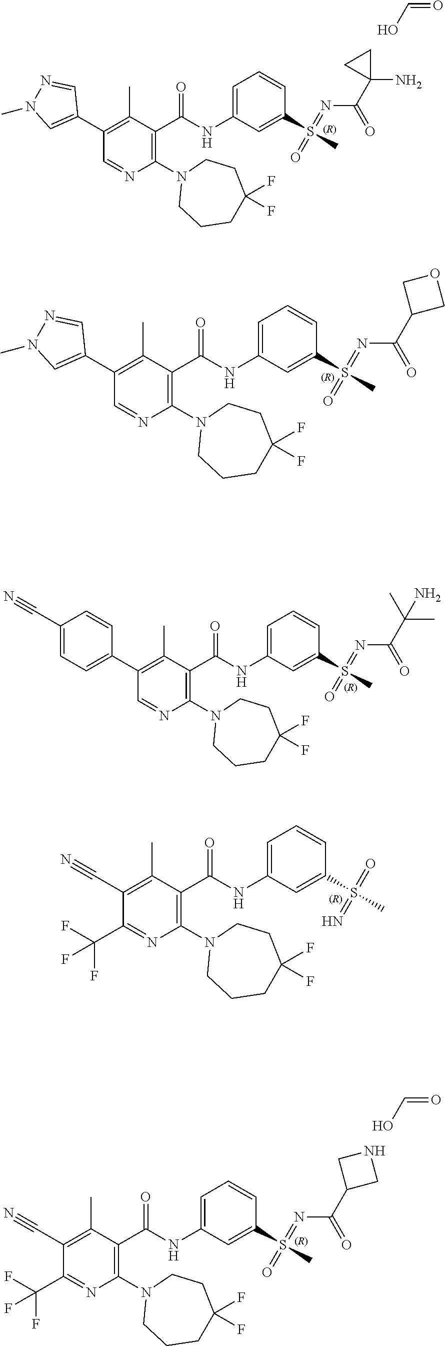

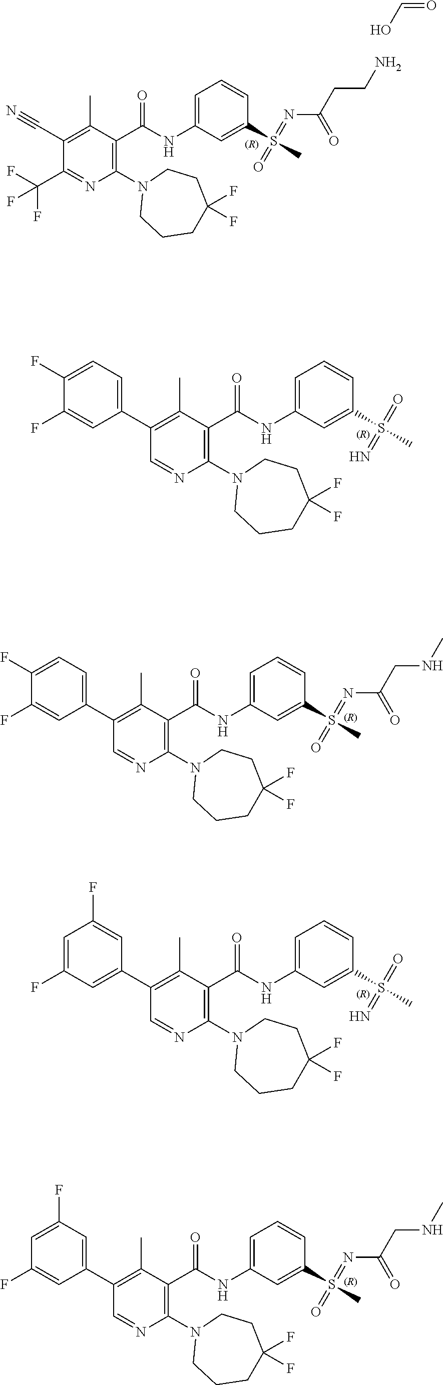

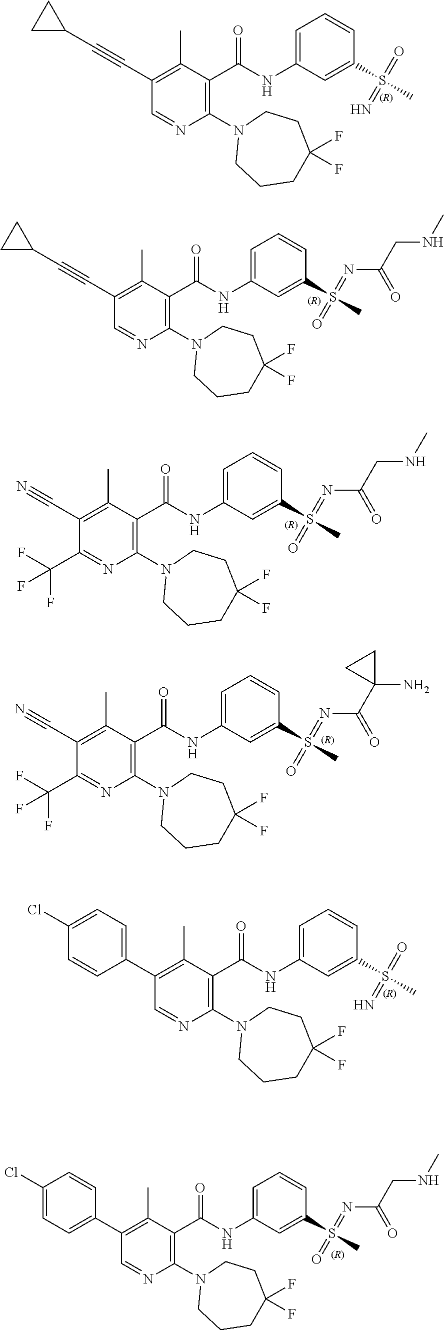

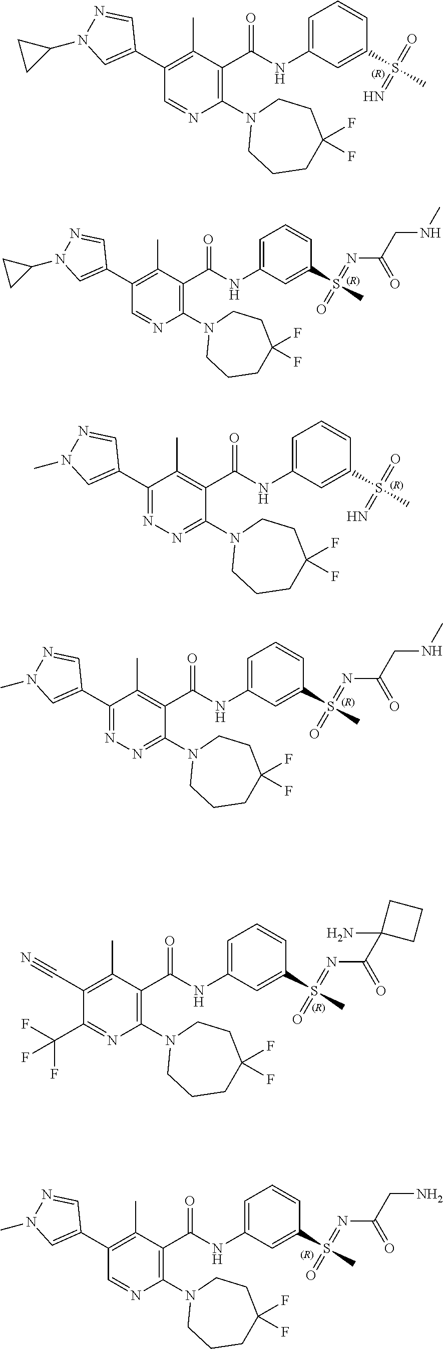

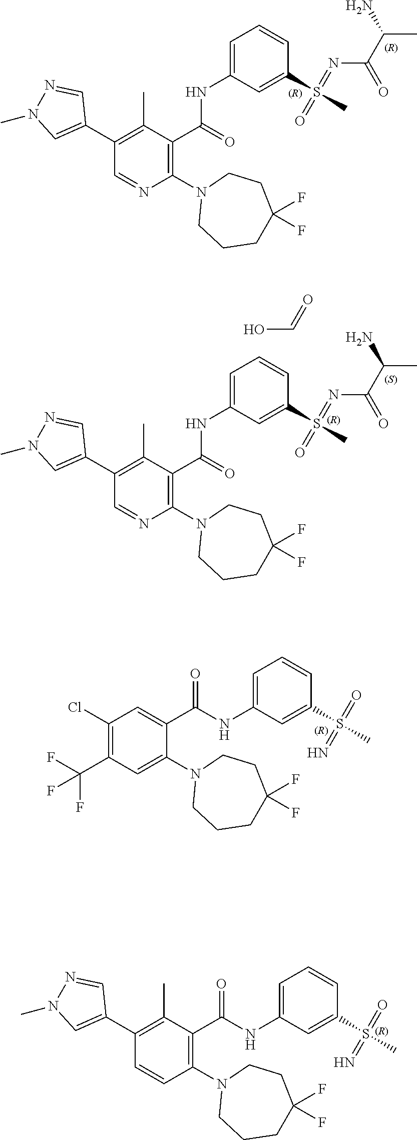

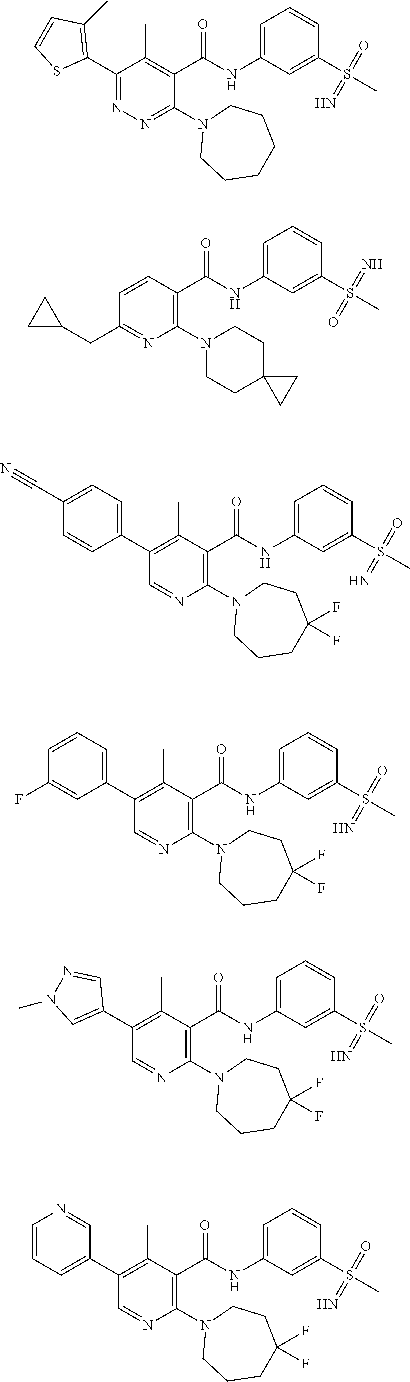

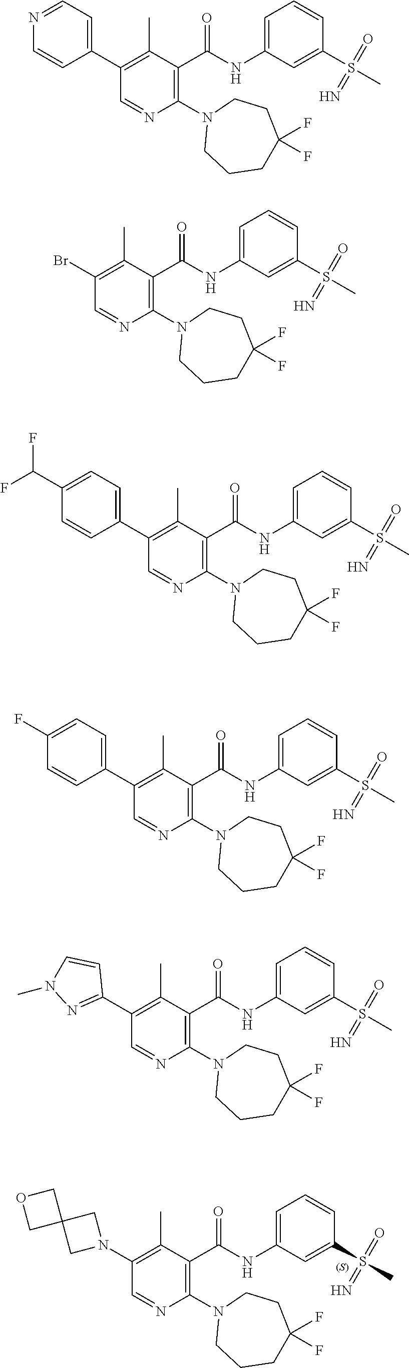

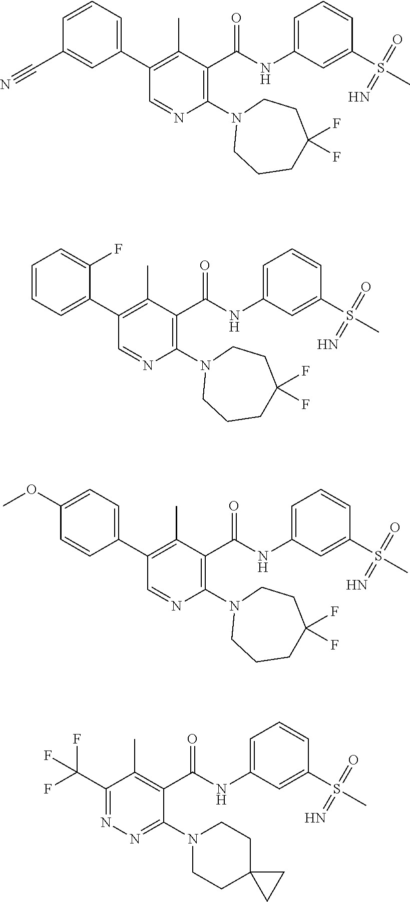

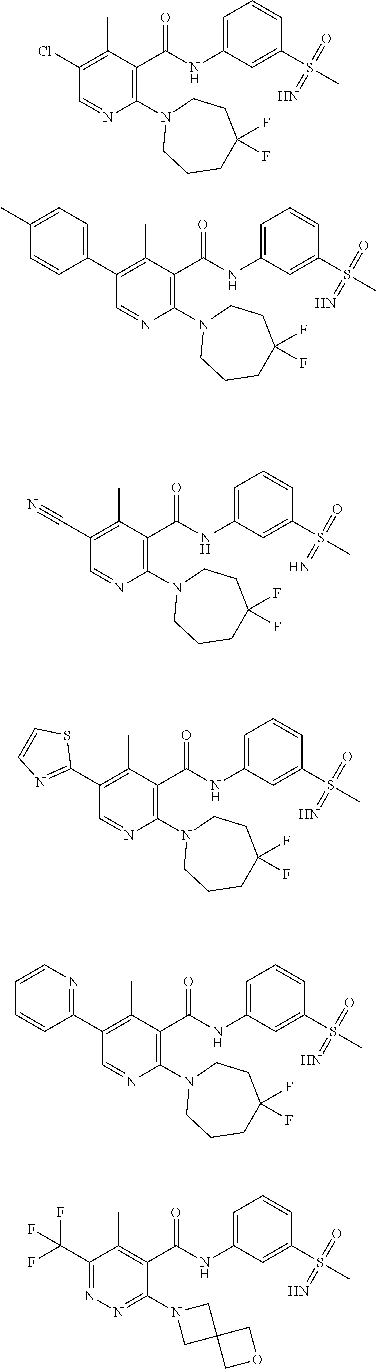

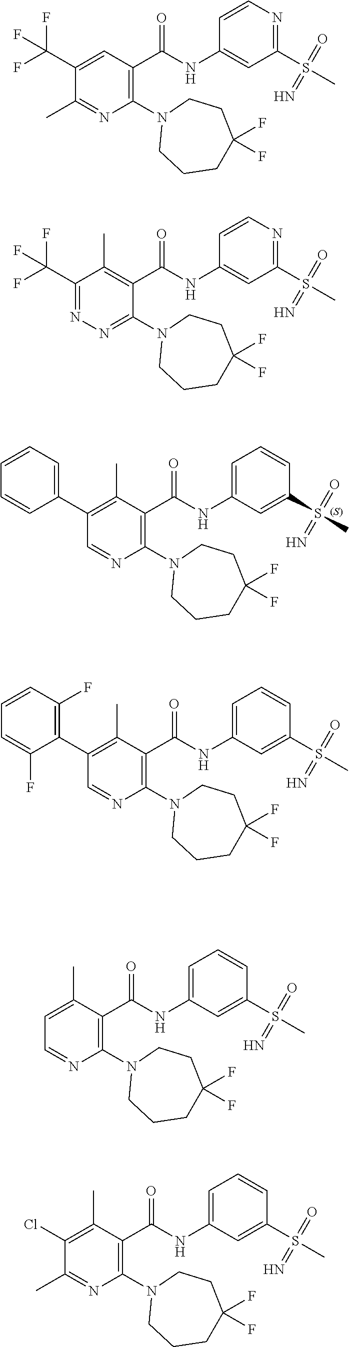

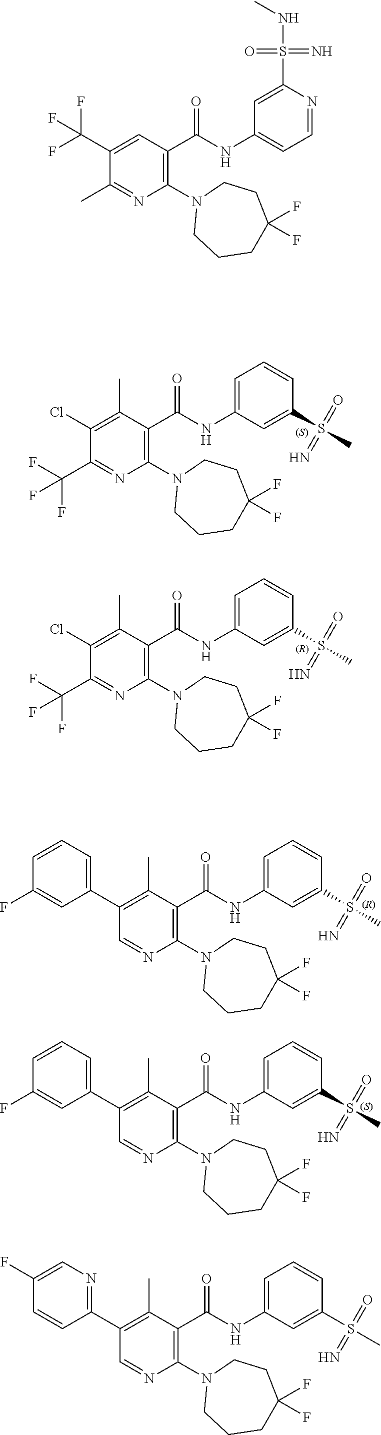

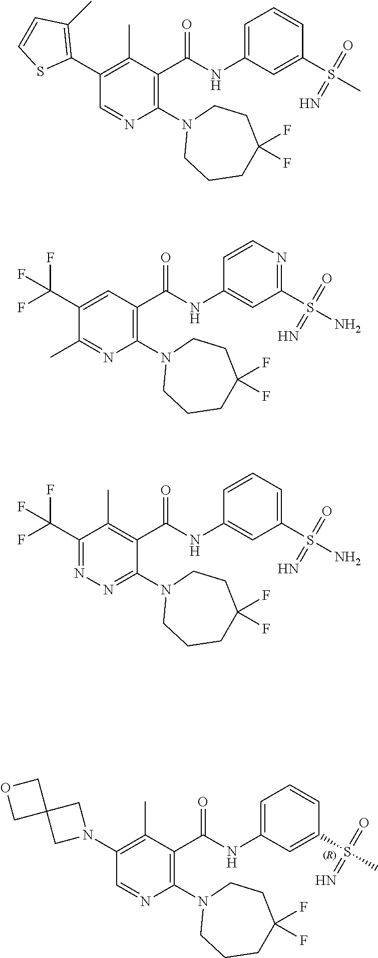

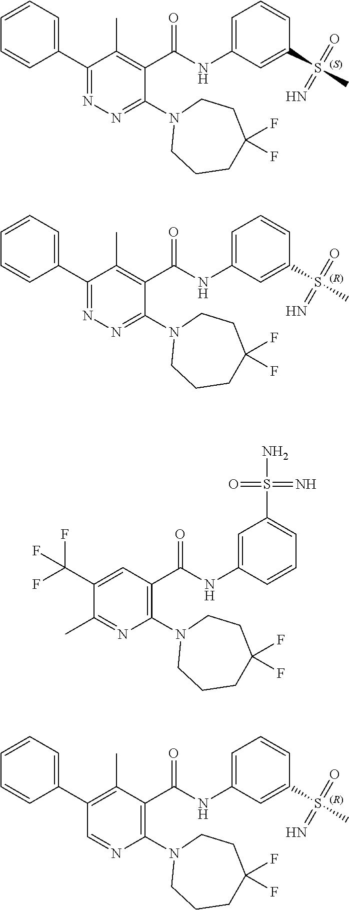

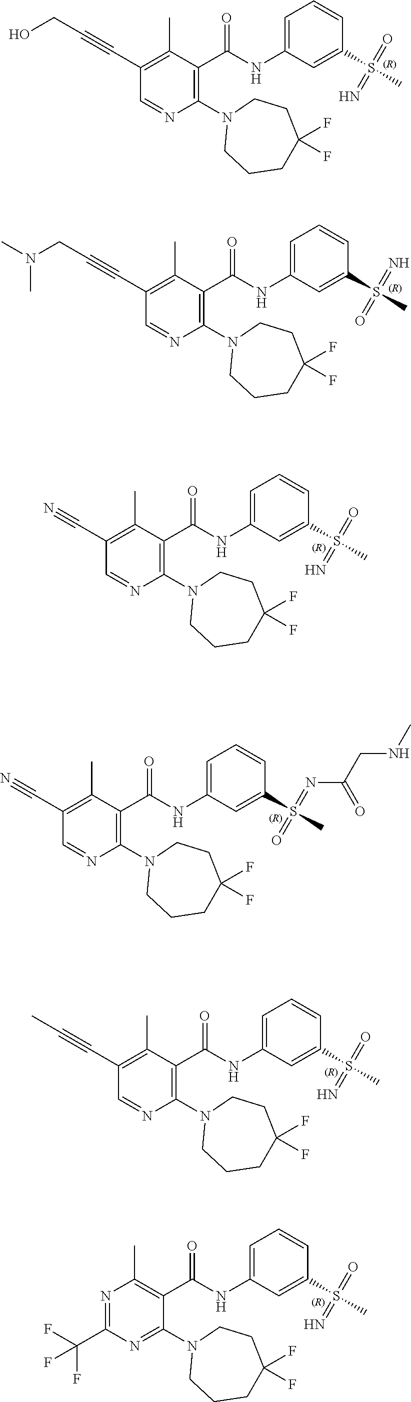

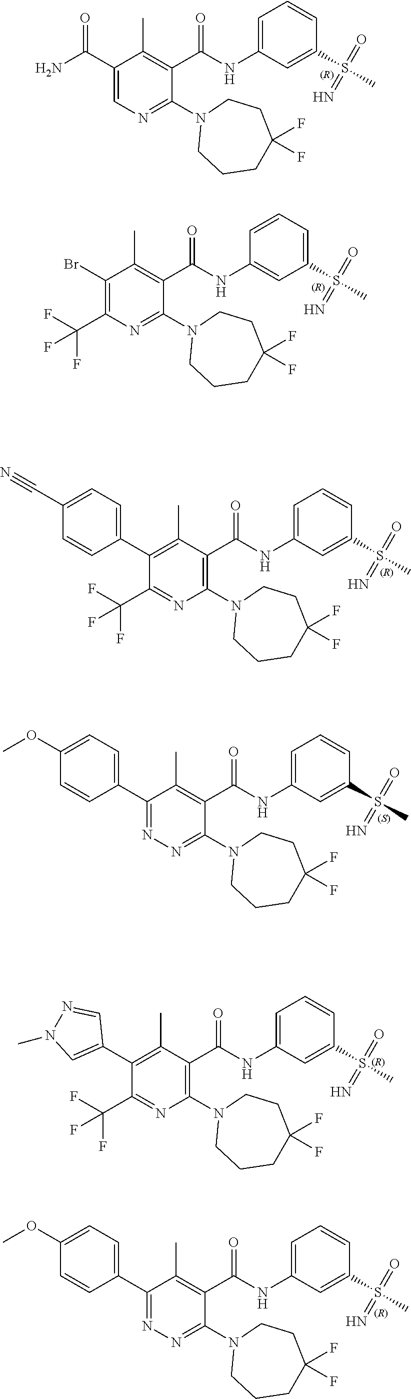

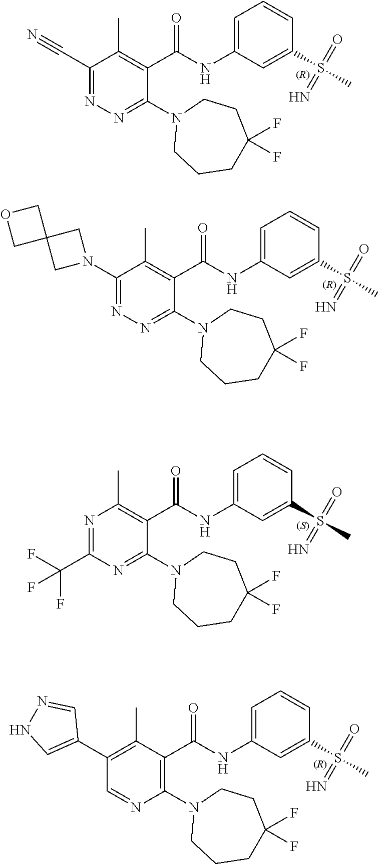

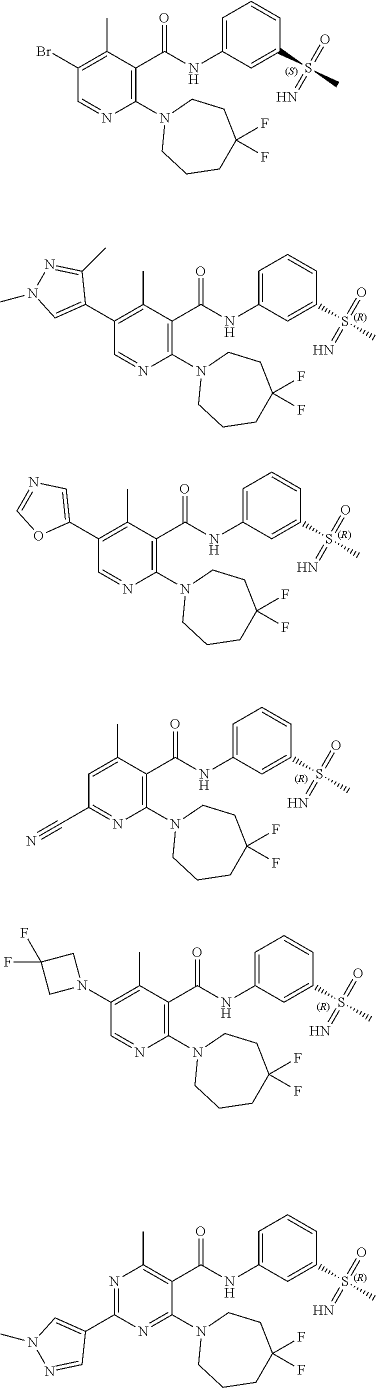

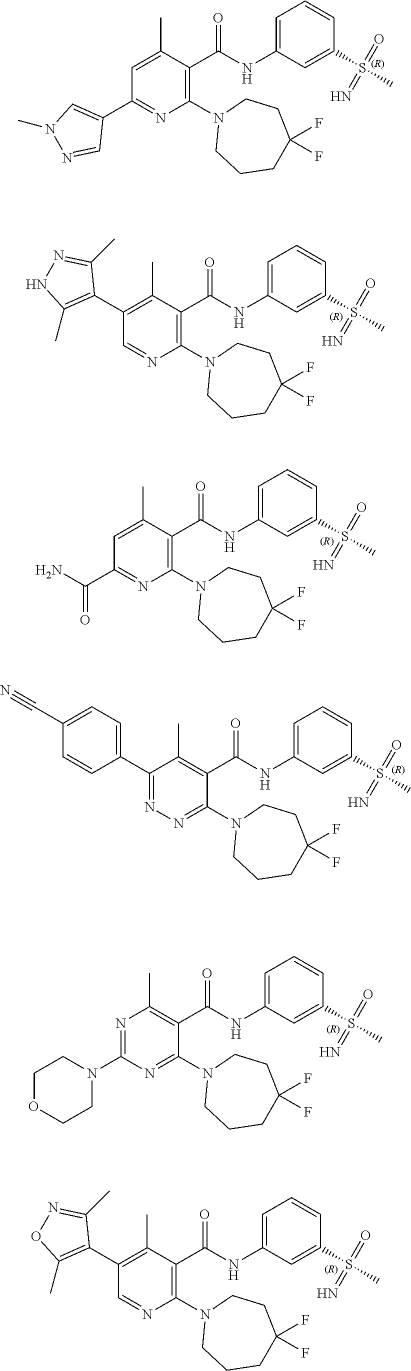

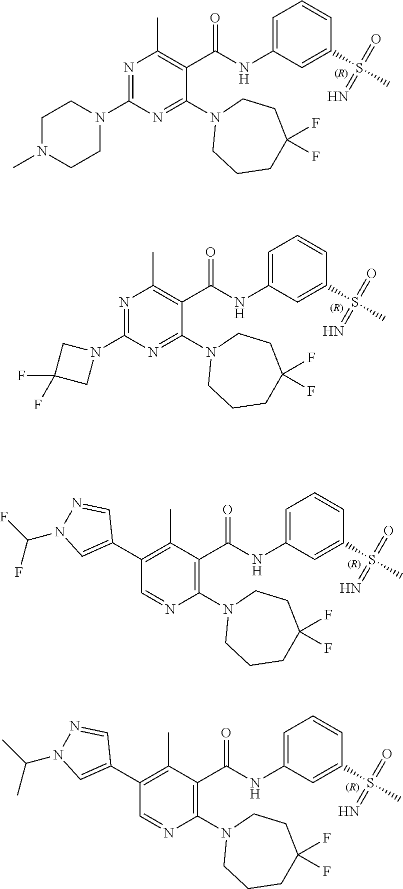

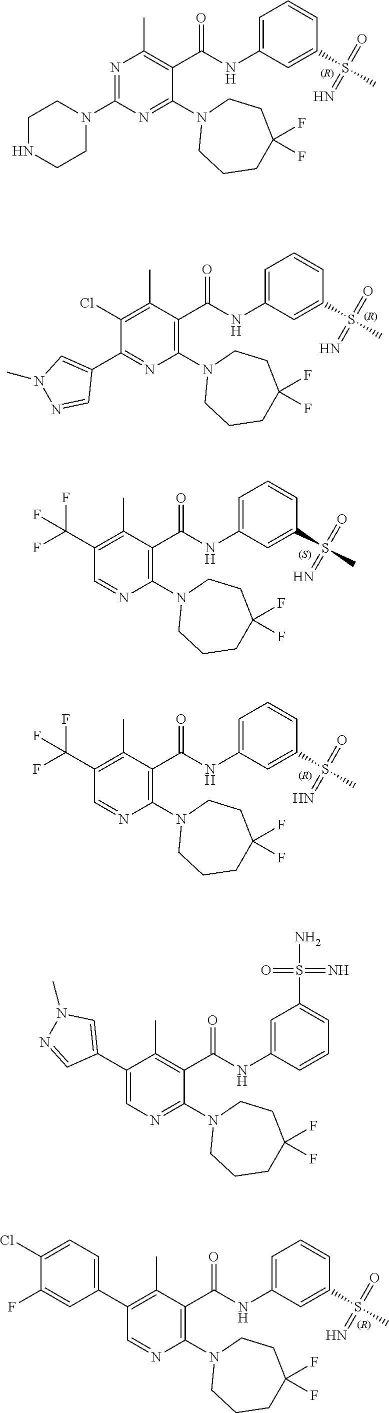

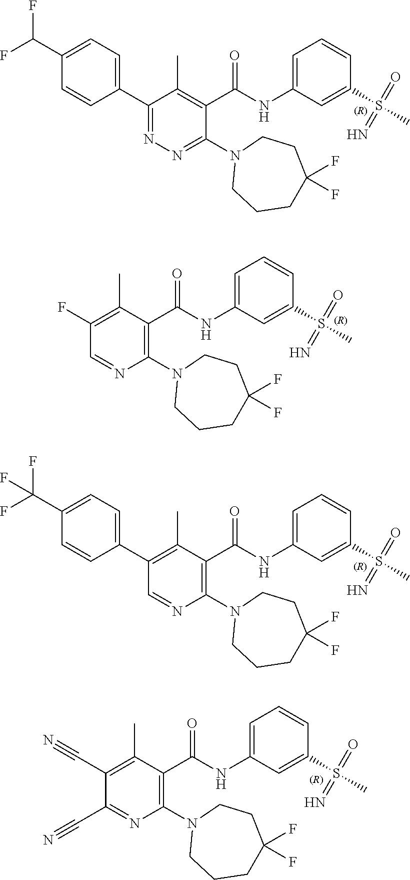

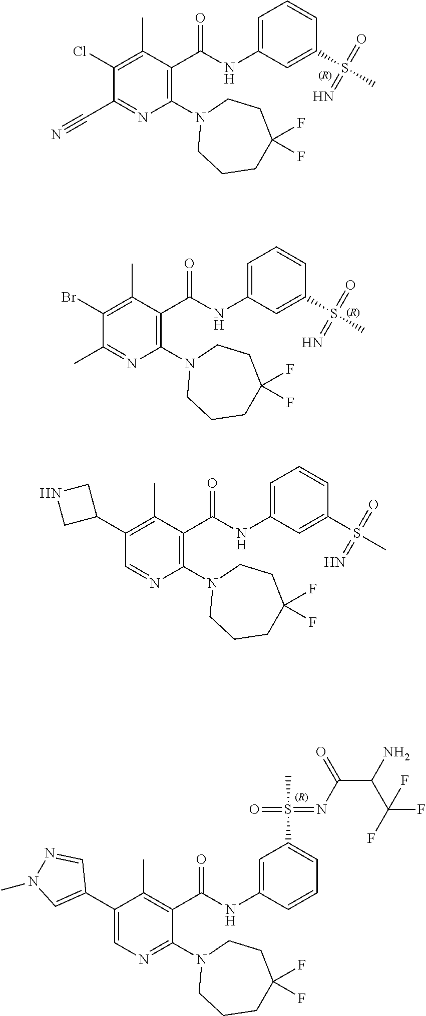

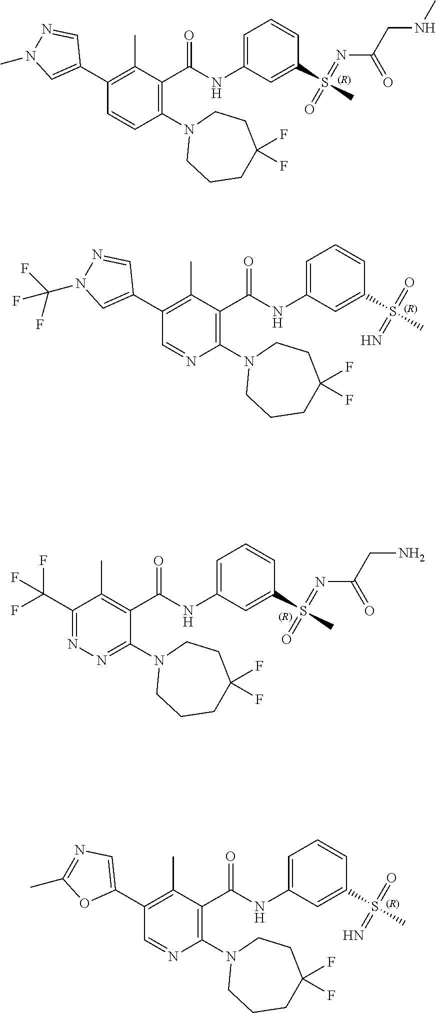

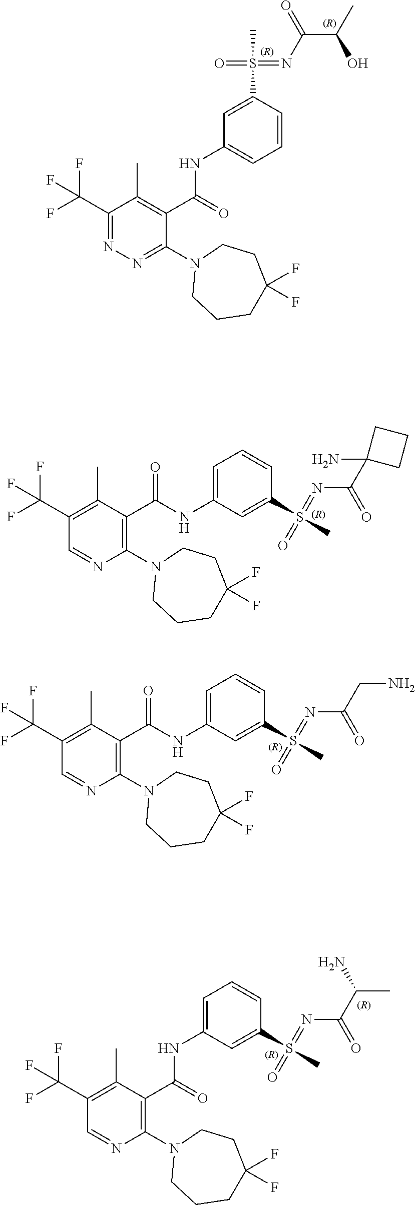

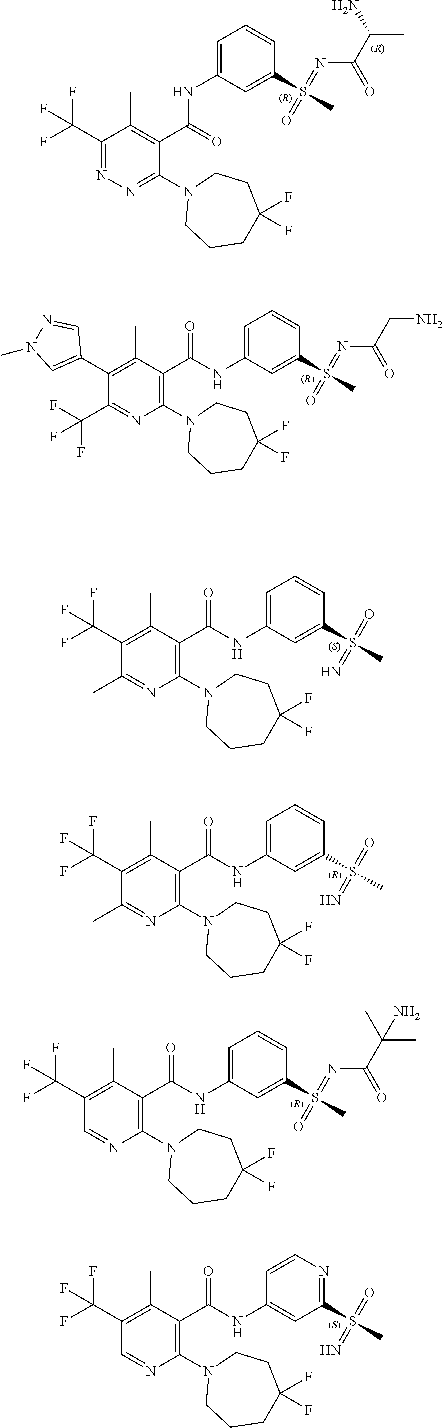

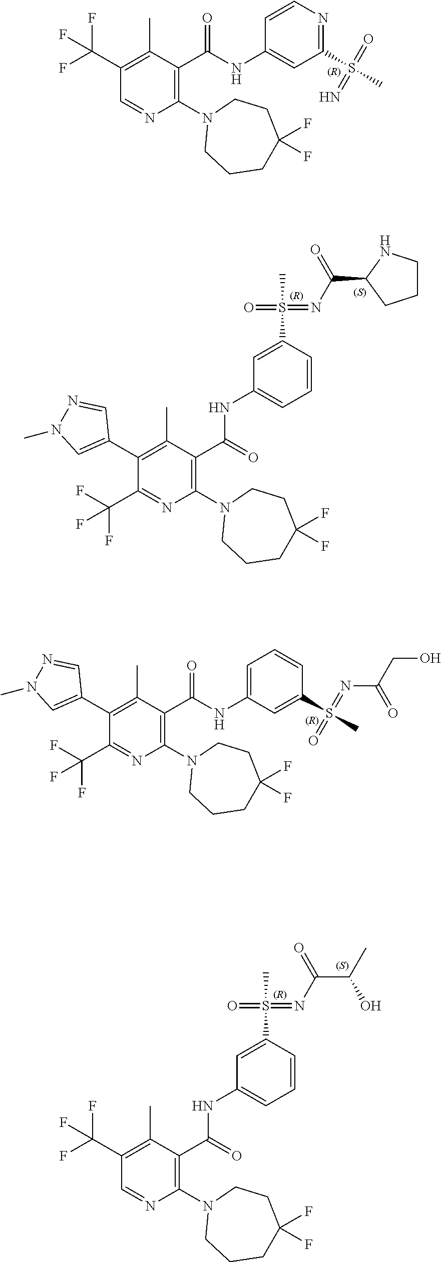

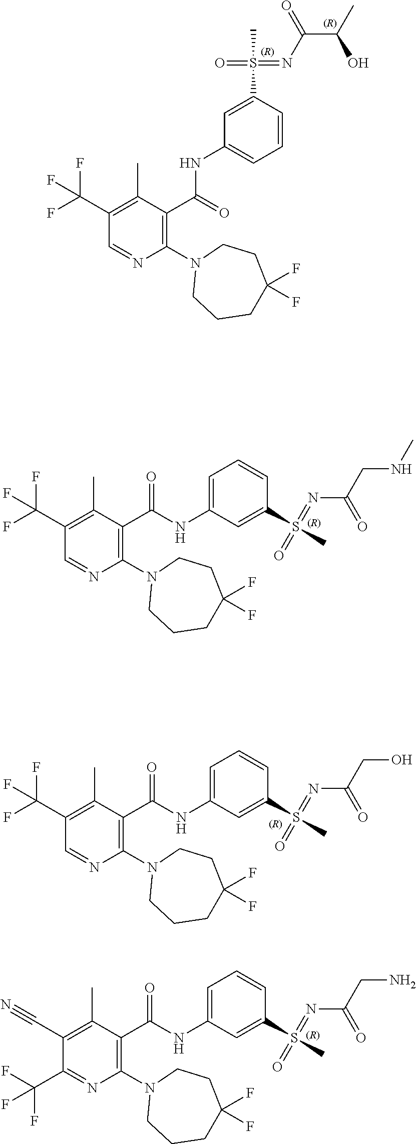

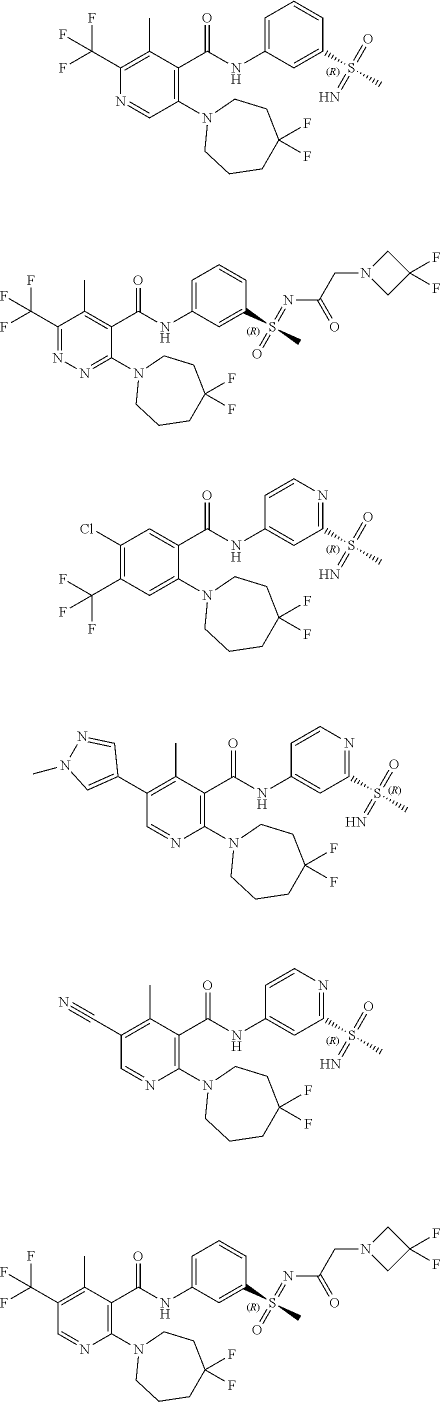

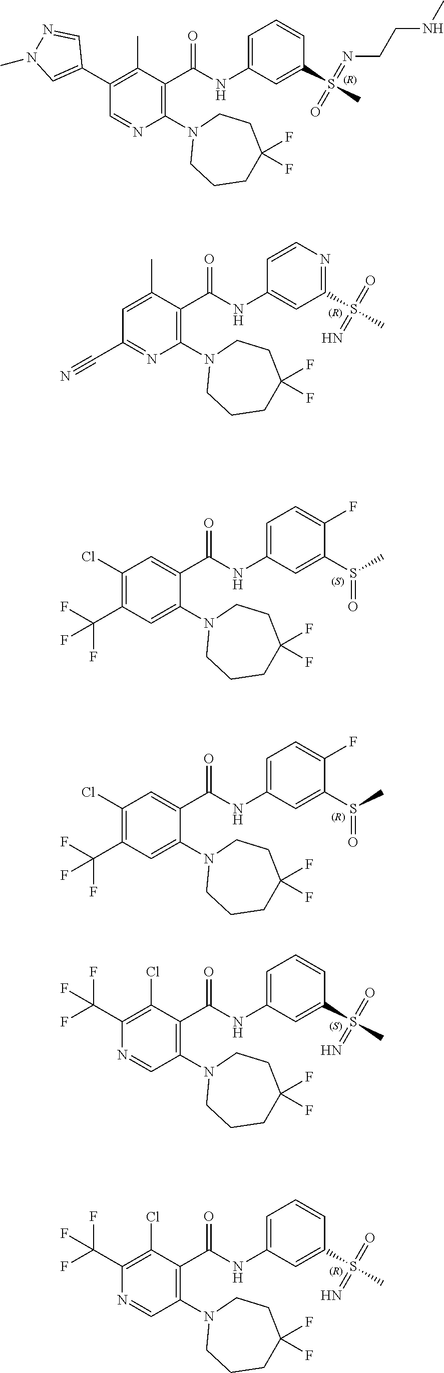

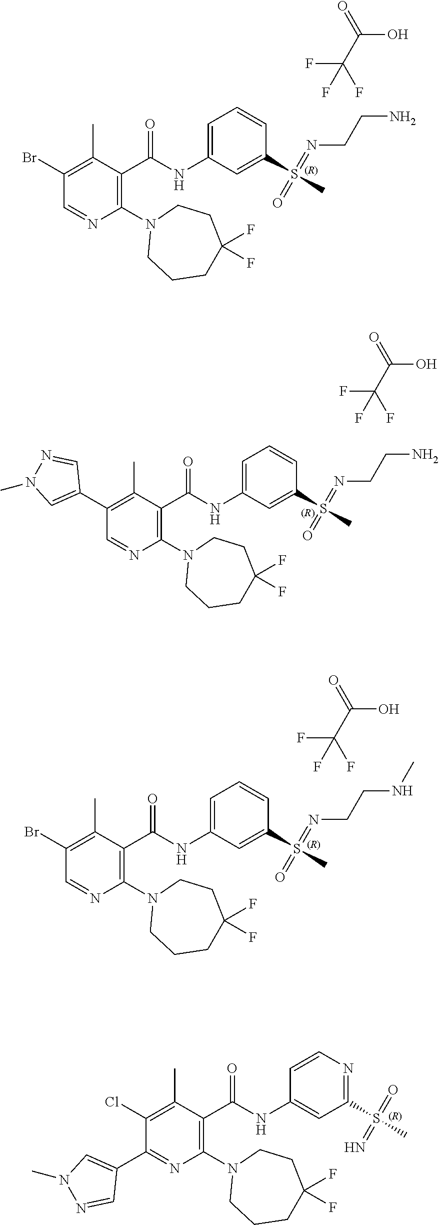

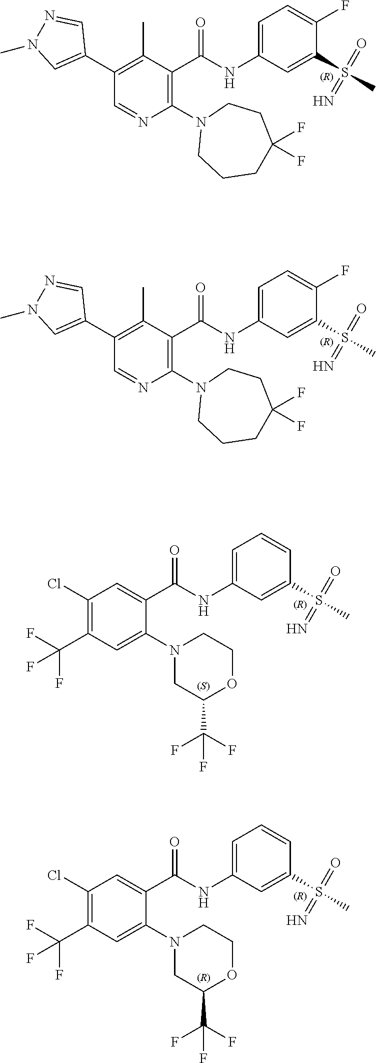

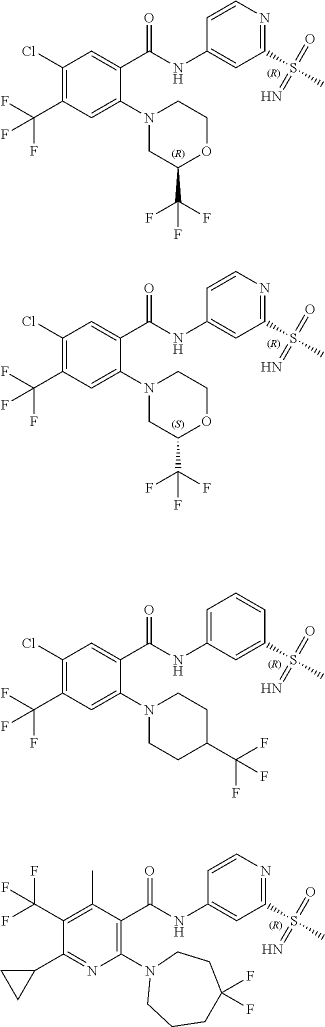

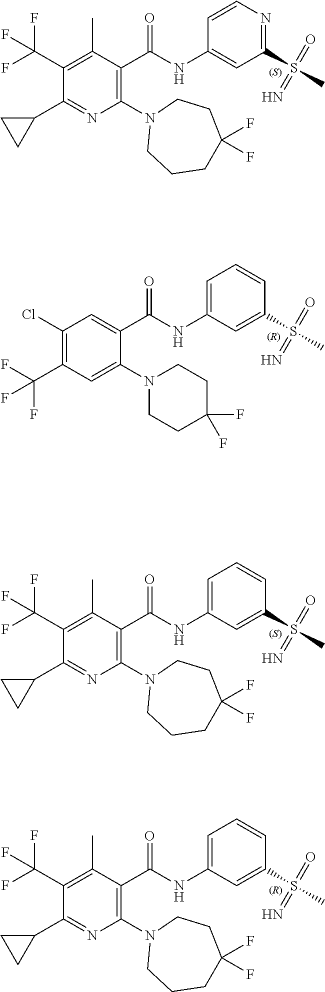

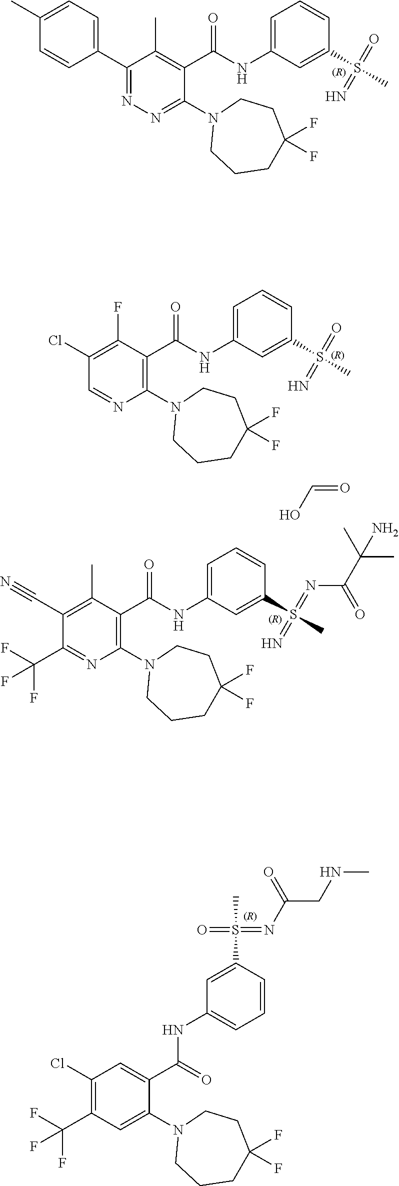

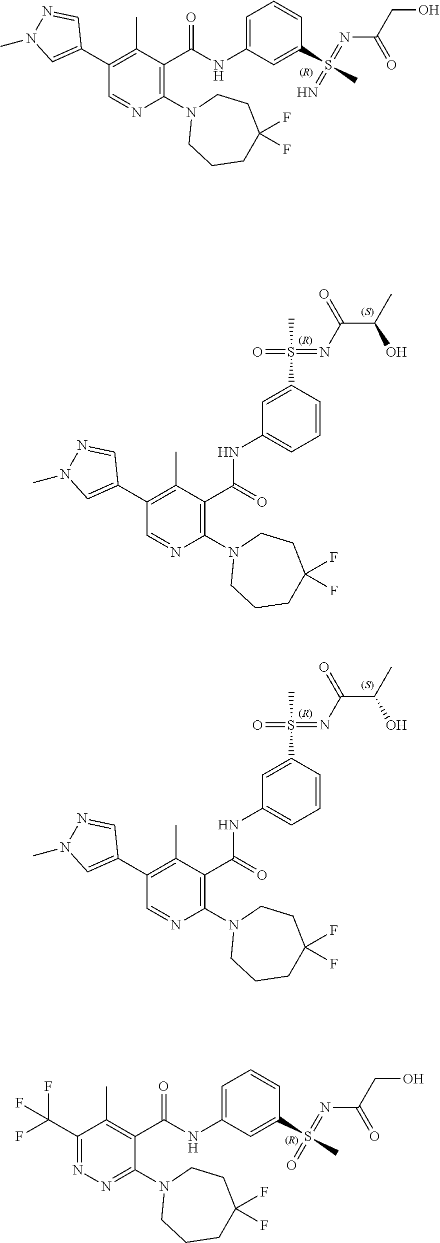

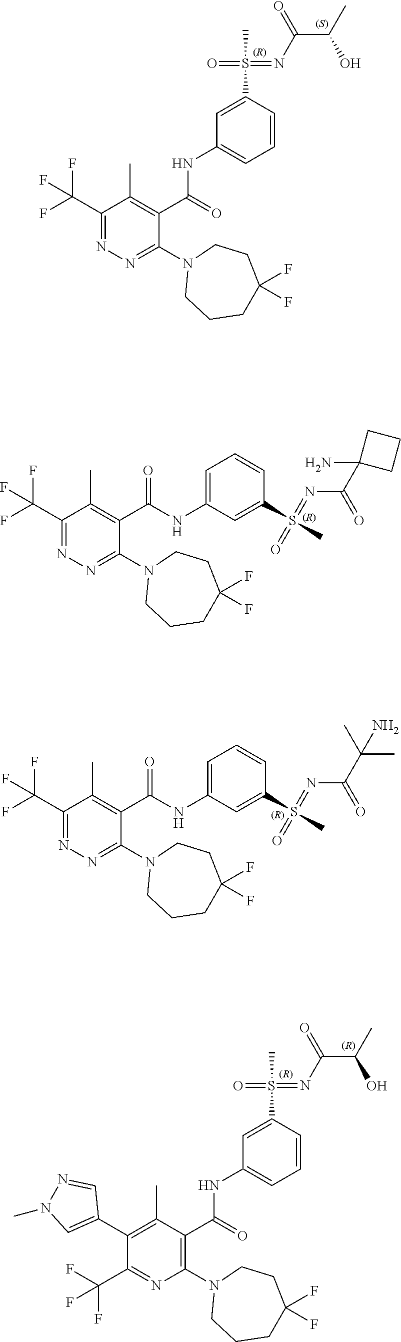

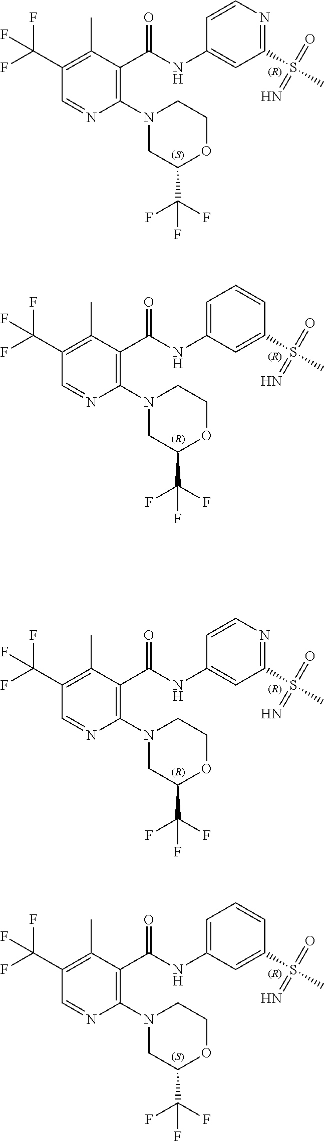

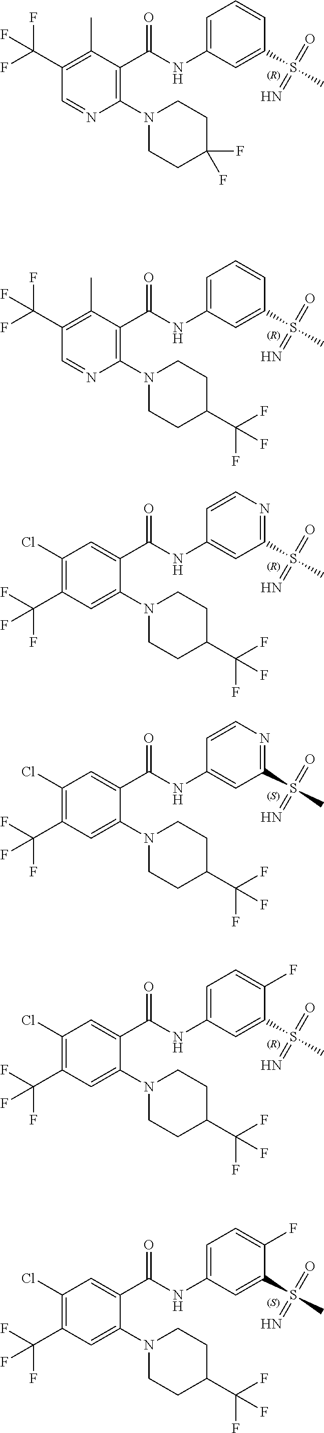

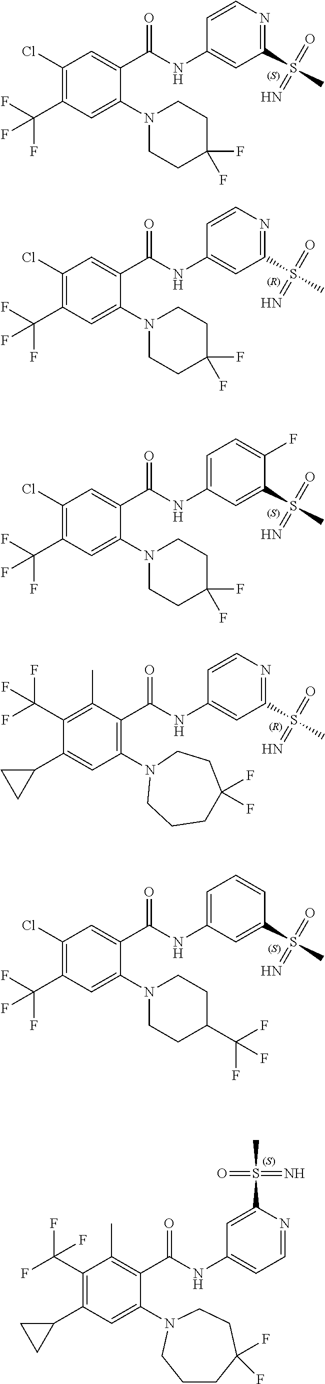

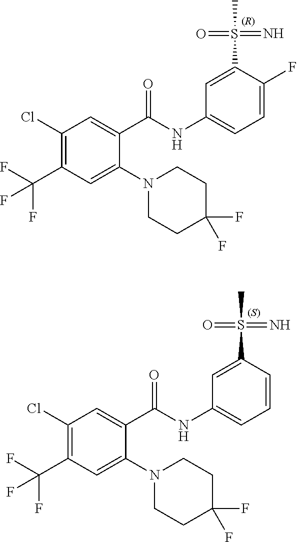

In certain embodiments, the compound of the invention is selected from a group consisting of:

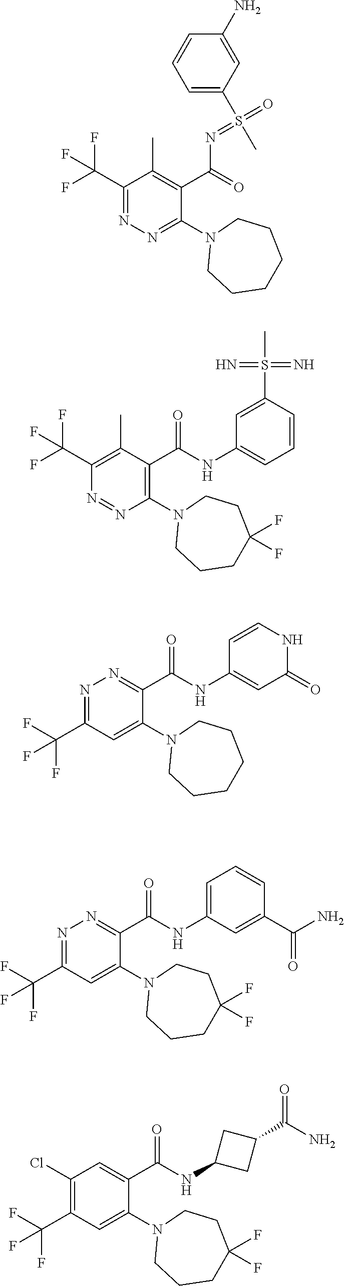

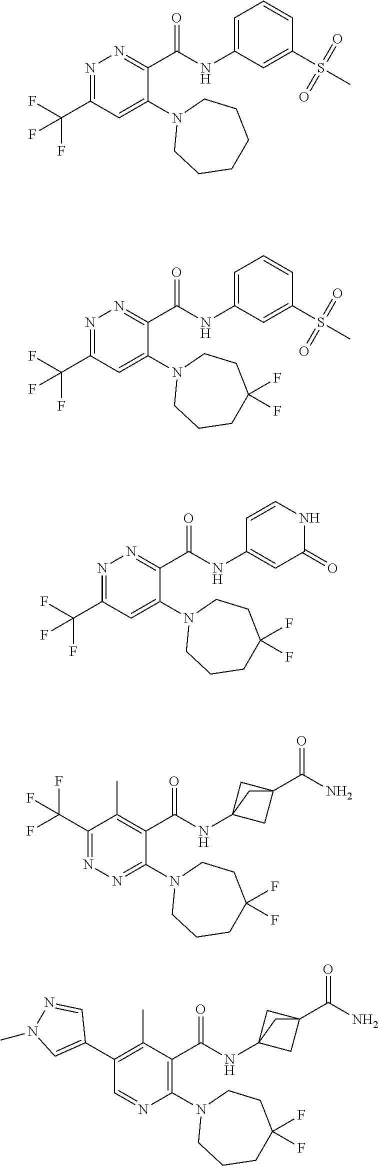

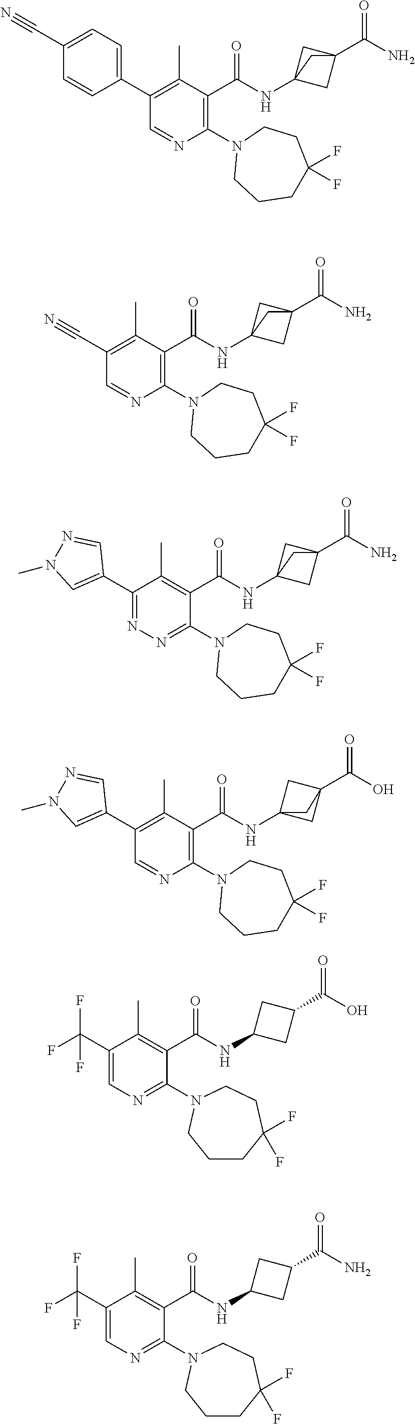

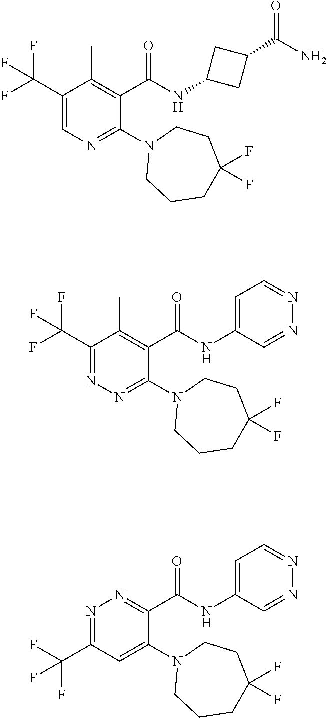 [SMILES: Cc1c(C(F)(F)F)cnc(N2CCCC(F)(F)CC2)c1C(=O)N[C@H]1C[C@@H](C(N)=O)C1 ; Cc1c(C(F)(F)F)nnc(N2CCCC(F)(F)CC2)c1C(=O)Nc1ccnnc1 ; O=C(Nc1ccnnc1)c1nnc(C(F)(F)F)cc1N1CCCC(F)(F)CC1]

In certain embodiments, the compound of the invention is selected from a group consisting of:

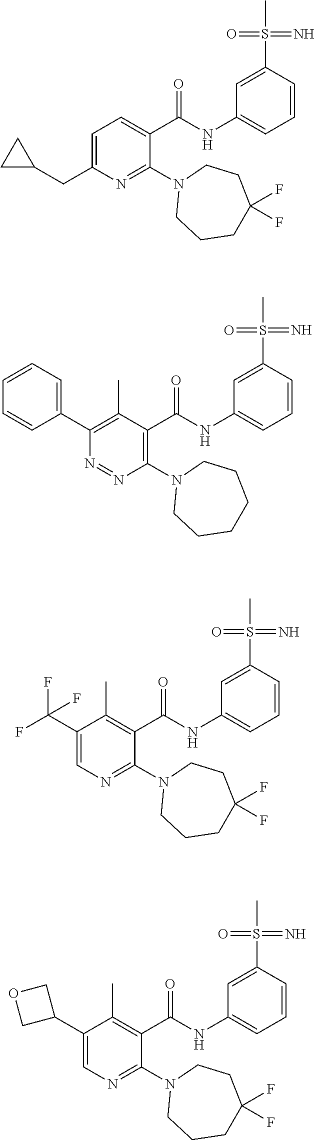 [SMILES: CS(=N)(=O)c1cccc(NC(=O)c2ccc(CC3CC3)nc2N2CCCC(F)(F)CC2)c1 ; Cc1c(-c2ccccc2)nnc(N2CCCCCC2)c1C(=O)Nc1cccc(S(C)(=N)=O)c1 ; Cc1c(C(F)(F)F)cnc(N2CCCC(F)(F)CC2)c1C(=O)Nc1cccc(S(C)(=N)=O)c1 ; Cc1c(C2COC2)cnc(N2CCCC(F)(F)CC2)c1C(=O)Nc1cccc(S(C)(=N)=O)c1]

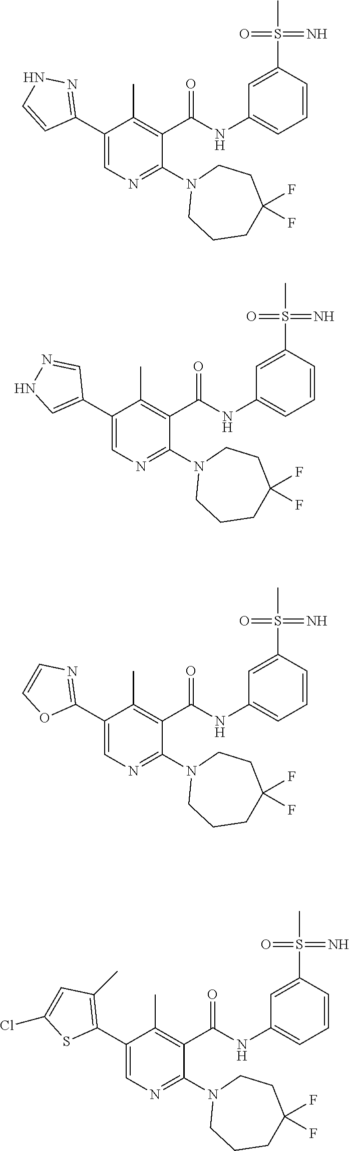 [SMILES: Cc1c(-c2cc[nH]n2)cnc(N2CCCC(F)(F)CC2)c1C(=O)Nc1cccc(S(C)(=N)=O)c1 ; Cc1c(-c2cn[nH]c2)cnc(N2CCCC(F)(F)CC2)c1C(=O)Nc1cccc(S(C)(=N)=O)c1 ; Cc1c(-c2ncco2)cnc(N2CCCC(F)(F)CC2)c1C(=O)Nc1cccc(S(C)(=N)=O)c1 ; Cc1cc(Cl)sc1-c1cnc(N2CCCC(F)(F)CC2)c(C(=O)Nc2cccc(S(C)(=N)=O)c2)c1C]

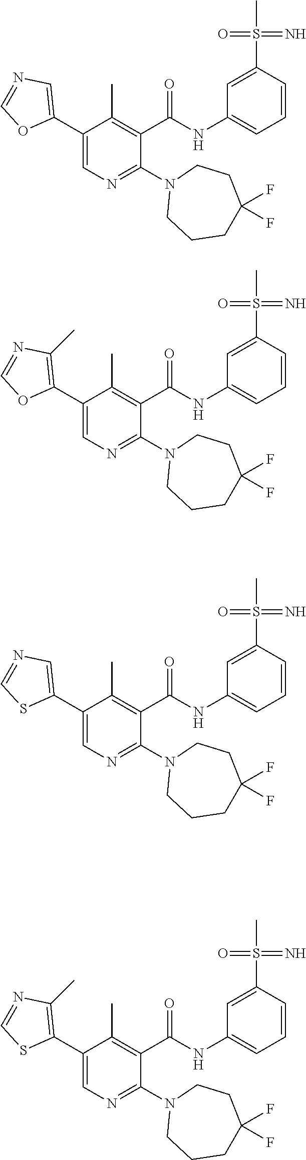 [SMILES: Cc1c(-c2cnco2)cnc(N2CCCC(F)(F)CC2)c1C(=O)Nc1cccc(S(C)(=N)=O)c1 ; Cc1ncoc1-c1cnc(N2CCCC(F)(F)CC2)c(C(=O)Nc2cccc(S(C)(=N)=O)c2)c1C ; Cc1c(-c2cncs2)cnc(N2CCCC(F)(F)CC2)c1C(=O)Nc1cccc(S(C)(=N)=O)c1 ; Cc1ncsc1-c1cnc(N2CCCC(F)(F)CC2)c(C(=O)Nc2cccc(S(C)(=N)=O)c2)c1C]

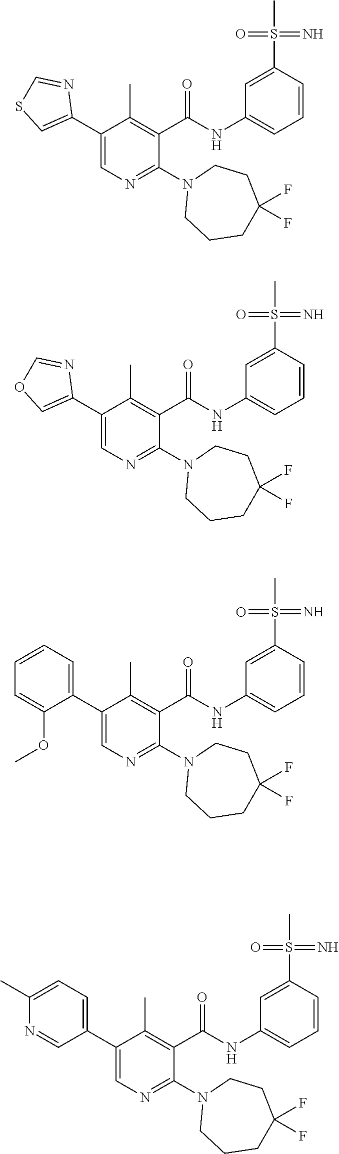 [SMILES: Cc1c(-c2cscn2)cnc(N2CCCC(F)(F)CC2)c1C(=O)Nc1cccc(S(C)(=N)=O)c1 ; Cc1c(-c2cocn2)cnc(N2CCCC(F)(F)CC2)c1C(=O)Nc1cccc(S(C)(=N)=O)c1 ; COc1ccccc1-c1cnc(N2CCCC(F)(F)CC2)c(C(=O)Nc2cccc(S(C)(=N)=O)c2)c1C ; Cc1ccc(-c2cnc(N3CCCC(F)(F)CC3)c(C(=O)Nc3cccc(S(C)(=N)=O)c3)c2C)cn1]

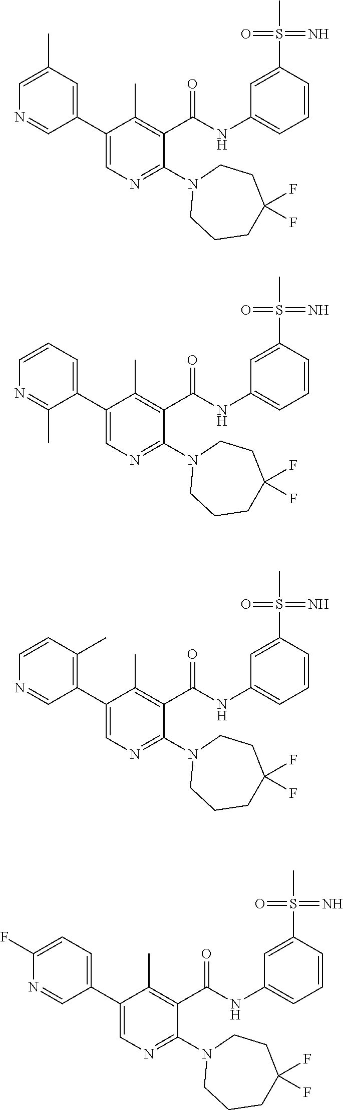 [SMILES: Cc1cncc(-c2cnc(N3CCCC(F)(F)CC3)c(C(=O)Nc3cccc(S(C)(=N)=O)c3)c2C)c1 ; Cc1ncccc1-c1cnc(N2CCCC(F)(F)CC2)c(C(=O)Nc2cccc(S(C)(=N)=O)c2)c1C ; Cc1ccncc1-c1cnc(N2CCCC(F)(F)CC2)c(C(=O)Nc2cccc(S(C)(=N)=O)c2)c1C ; Cc1c(-c2ccc(F)nc2)cnc(N2CCCC(F)(F)CC2)c1C(=O)Nc1cccc(S(C)(=N)=O)c1]

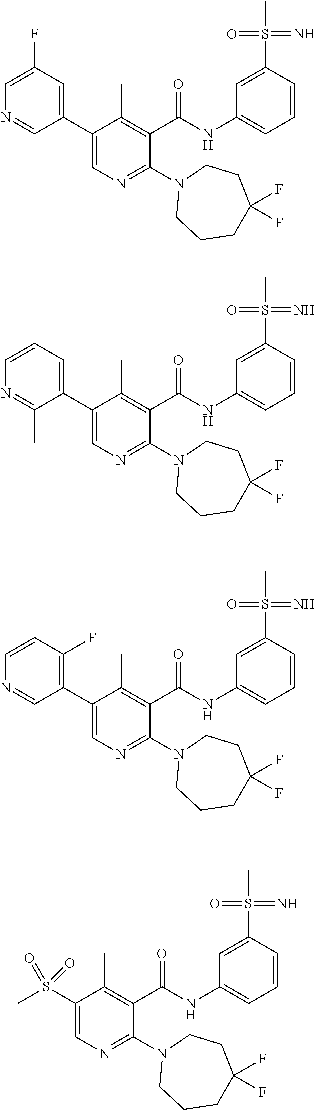 [SMILES: Cc1c(-c2cncc(F)c2)cnc(N2CCCC(F)(F)CC2)c1C(=O)Nc1cccc(S(C)(=N)=O)c1 ; Cc1ncccc1-c1cnc(N2CCCC(F)(F)CC2)c(C(=O)Nc2cccc(S(C)(=N)=O)c2)c1C ; Cc1c(-c2cnccc2F)cnc(N2CCCC(F)(F)CC2)c1C(=O)Nc1cccc(S(C)(=N)=O)c1 ; Cc1c(S(C)(=O)=O)cnc(N2CCCC(F)(F)CC2)c1C(=O)Nc1cccc(S(C)(=N)=O)c1]

### Compositions

The invention provides pharmaceutical compositions containing compounds of the inventions, such as those described above. The pharmaceutical composition may be in a form suitable for oral use, for example, as tablets, troches, lozenges, fast-melts, aqueous or oily suspensions, dispersible powders or granules, emulsions, hard or soft capsules, syrups, or elixirs. Compositions intended for oral use may be prepared according to any method known in the art for the manufacture of pharmaceutical compositions and such compositions may contain one or more agents selected from sweetening agents, flavoring agents, coloring agents, and preserving agents, to provide pharmaceutically elegant and palatable preparations. Tablets contain the compounds in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch, or alginic acid; binding agents, for example starch, gelatin or acacia, and lubricating agents, for example magnesium stearate, stearic acid, or talc. The tablets may be uncoated, or they may be coated by known techniques to delay disintegration in the stomach and absorption lower down in the gastrointestinal tract and thereby provide a sustained action over a longer period. For example, a time delay material such as glyceryl monostearate or glyceryl distearate may be employed. They may also be coated by the techniques described in U.S. Pat. Nos. 4,256,108; 4,166,452; and 4,265,874, the contents of which are incorporated herein by reference, to form osmotic therapeutic tablets for control release. Preparation and administration of compounds is discussed in U.S. Pat. No. 6,214,841 and U.S. Pub. No. 2003/0232877, the contents of which are incorporated herein by reference.

Formulations for oral use may also be presented as hard gelatin capsules in which the compounds are mixed with an inert solid diluent, for example calcium carbonate, calcium phosphate or kaolin, or as soft gelatin capsules in which the compounds are mixed with water or an oil medium, for example peanut oil, liquid paraffin, or olive oil.

An alternative oral formulation, where control of gastrointestinal tract hydrolysis of the compound is sought, can be achieved using a controlled-release formulation, where a compound of the invention is encapsulated in an enteric coating.

Aqueous suspensions may contain the compounds in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents such as a naturally occurring phosphatide, for example lecithin, or condensation products of an alkylene oxide with fatty acids, for example, polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such a polyoxyethylene with partial esters derived from fatty acids and hexitol anhydrides, for example polyoxyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the compounds in a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the compounds in admixture with a dispersing or wetting agent, suspending agent and one or more preservatives. Suitable dispersing or wetting agents and suspending agents are exemplified, for example sweetening, flavoring, and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavoring agents.

Syrups and elixirs may be formulated with sweetening agents, such as glycerol, propylene glycol, sorbitol, or sucrose. Such formulations may also contain a demulcent, a preservative, and agents for flavoring and/or coloring. The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be in a sterile injectable solution or suspension in a non-toxic parenterally acceptable diluent or solvent, for example as a solution in 1,3-butanediol. Among the acceptable vehicles and solvents that may be employed are water, Ringer [symbol] solution, and isotonic sodium chloride solution. In addition, sterile, fixed oils are conventionally employed as a solvent or suspending medium. For this purpose, any bland fixed oil may be employed, including synthetic mono- or di-glycerides. In addition, fatty acids such as oleic acid find use in the preparation of injectables.

In certain embodiments, the formulation is a sustained release formulation. In certain embodiments, the formulation is not a sustained release formulation. In certain embodiments, the formulation is not injectable. In certain embodiments, the formulation does not contain particles having a D₅₀ (volume weighted median diameter) of less than 10 microns. In certain embodiments, the formulation does not contain a polymer surface stabilizer. In certain embodiments, the formulation is not an aqueous suspension.

The composition may be formulated for administration by a particular mechanism. The composition may be formulated for oral, intravenous, enteral, parenteral, dermal, buccal, topical, nasal, or pulmonary administration. The composition may be formulated for administration by injection or on an implantable medical device (e.g., stent or drug-eluting stent or balloon equivalents).

The composition may be formulated a single daily dosage. The composition may be formulated for multiple daily dosages, e.g., two, three, four, five, six or more daily dosages.

### Methods of Treating Conditions

The invention provides methods of treating a condition in a subject using compounds of the invention. The methods are useful for treating any condition associated with aberrant, e.g., increased, activity of voltage gated sodium channel Nav1.8. Conditions associated with increased activity at voltage gated sodium channels and the use of inhibitors to treat such conditions is known in the art and described in, for example, International Patent Publication Nos. WO 2020/014243, WO 2020/014246, WO 2020/092187, the contents of each of which are incorporated herein by reference.

For example and without limitation, the condition may be abdominal cancer pain, acute cough, acute idiopathic transverse myelitis, acute itch, acute pain, acute pain in major trauma/injury, airways hyperreactivity, allergic dermatitis, allergies, ankylosing spondylitis, asthma, atopy, Behcet [symbol] disease, bladder pain syndrome, bone cancer pain, brachial plexus injury, burn injury, burning mouth syndrome, calcium pyrophosphate deposition disease, cervicogenic headache, Charcot neuropathic osteoarthropathy, chemotherapy-induced oral mucositis, chemotherapy-induced peripheral neuropathy, cholestasis, chronic cough, chronic itch, chronic low back pain, chronic pain, chronic pancreatitis, chronic post-traumatic headache, chronic widespread pain, cluster headache, complex regional pain syndrome, complex regional pain syndromes, constant unilateral facial pain with additional attacks, contact dermatitis, cough, dental pain, diabetic neuropathy, diabetic peripheral neuropathy, diffuse idiopathic skeletal hyperostosis, disc degeneration pain, distal sensory polyneuropathy (DSP) associated with highly active antiretroviral therapy (HAART), Ehlers-Danlos syndrome, endometriosis, epidermolysis bullosa, epilepsy, erythromelalgia, Fabry [symbol] disease, facet joint syndrome, failed back surgery syndrome, familial hemiplegic migraine, fibromyalgia, glossopharyngeal neuralgia, glossopharyngeal neuropathic pain, gout, head and neck cancer pain, inflammatory bowel disease, inflammatory pain, inherited erythromelalgia, irritable bowel syndrome, irritable bowel syndrome, itch, juvenile idiopathic arthritis, mastocytosis, melorheostosis, migraine, multiple sclerosis, musculoskeletal damage, myofascial orofacial pain, neurodegeneration following ischemia, neurofibromatosis type II, neuropathic ocular pain, neuropathic pain, neuropathic pain, nociceptive pain, non-cardiac chest pain, optic neuritis, oral mucosal pain, orofacial pain, osteoarthritis, osteoarthritis, overactive bladder, pachyonychia congenita, pain, pain resulting from cancer, pain resulting from chemotherapy, pain resulting from diabetes, pain syndrome, painful joint arthroplasties, pancreatitis, Parkinson [symbol] disease, paroxysmal extreme pain disorder, pemphigus, perioperative pain, peripheral neuropathy, persistent idiopathic dentoalveolar pain, persistent idiopathic facial pain, phantom limb pain, phantom limb pain, polymyalgia rheumatica, postherpetic neuralgia, post-mastectomy pain syndrome, postoperative pain, post-stroke pain, post-surgical pain, post-thoracotomy pain syndrome, post-traumatic stress disorder, preoperative pain, pruritus, psoriasis, psoriatic arthritis, pudendal neuralgia, pyoderma gangrenosum, radiotherapy-induced peripheral neuropathy, Raynaud [symbol] disease, renal colic, renal colic, renal failure, rheumatoid arthritis, salivary gland pain, sarcoidosis, sciatica, scleroderma, sickle cell disease, small fiber neuropathy, spinal cord injury pain, spondylolisthesis, spontaneous pain, stump pain, subacute cough, temporomandibular joint disorders, tension-type headache, trigeminal neuralgia, vascular leg ulcers, vulvodynia, or whiplash associated disorder.

Methods of treating a condition in a subject may include providing a composition of the invention to a subject. The composition may be provided to a subject by any suitable route or mode of administration. For example and without limitation, the composition may be provided buccally, dermally, enterally, intraarterially, intramuscularly, intraocularly, intravenously, nasally, orally, parenterally, pulmonarily, rectally, subcutaneously, topically, transdermally, by injection, or with or on an implantable medical device.

The composition may be provided according to a dosing regimen. A dosing regimen may include one or more of a dosage, a dosing frequency, and a duration.

Doses may be provided at any suitable interval. For example and without limitation, doses may be provided once per day, twice per day, three times per day, four times per day, five times per day, six times per day, eight times per day, once every 48 hours, once every 36 hours, once every 24 hours, once every 12 hours, once every 8 hours, once every 6 hours, once every 4 hours, once every 3 hours, once every two days, once every three days, once every four days, once every five days, once every week, twice per week, three times per week, four times per week, or five times per week.

The dose may be provided in a single dosage, i.e., the dose may be provided as a single tablet, capsule, pill, etc. Alternatively, the dose may be provided in a divided dosage, i.e., the dose may be provided as multiple tablets, capsules, pills, etc.

The dosing may continue for a defined period. For example and without limitation, doses may be provided for at least 1 week, at least 2 weeks, at least 3 weeks, at least 4 weeks, at least 6 weeks, at least 8 weeks, at least 10 weeks, at least 12 weeks, at least 4 months, at least 5 months, at least 6 months, at least 8 months, at least 10 months, at least 12 months or more.

### EXAMPLES

Methods of making the compounds of the present invention, and intermediates used in their synthesis, are provided in the General Synthetic Schemes and Specific Syntheses Procedures below. Chemicals were purchased from standard commercial vendors and used as received unless otherwise noted. Otherwise, their preparation is facile and known to one of ordinary skill in the art, or it is referenced or described herein. Abbreviations are consistent with those in the ACS Style Guide. “dry” glassware means oven/desiccator dried. Solvents were ACS grade unless otherwise noted.

All reactions were performed in flame-dried or oven-dried glassware under a positive pressure of dry nitrogen or dry argon and were stirred magnetically unless otherwise indicated. Chemicals were purchased from standard commercial vendors and used as received unless otherwise noted. Yields are not optimized. The chemical names were generated using the ChemDraw Professional 19.1, available from PerkinElmer or chemAxon.

Reactions were monitored by thin layer chromatography (TLC) using 0.25 mm silica gel 60 F254 plates purchased from EMD MILLIPORE™. Purification was performed with Biotage Isolera One Flash Chromatography Instrument or purified using one of the preparative HPLC methods mentioned below.

### Prep Method 1

Equipment: Shimadzu LCMS 2020 mass-directed preparative HPLC System; column: Gemini 5 um C₁₈ column, 150*21.2 mm; General gradient: 30% to 90% MeCN/H₂O containing 0.1% HCOOH, gradient may be slight adjusted for specific compound; Flow rate: 20 mL/min; Column temperature: ambient temperature; UV Wavelength: 214 and 254 nm;

### Prep Method 2

Equipment: Shimadzu LC-20AP Preparative HPLC System; column: Gemini 5 um C₁₈ column, 150*21.2 mm; General gradient: 30% to 90% MeCN/H₂O containing 0.1% TFA, gradient may be slight adjusted for specific compound; Flow rate: 20 mL/min; Column temperature: ambient temperature; UV Wavelength: 214 and 254 nm.

### Prep Method 3

Equipment: Shimadzu LC-20AP Preparative HPLC System; column: Gemini 5 um C₁₈ column, 150×21.2 mm; General gradient: 30% to 90% MeCN/H₂O containing 0.05% ammonia, gradient may be slight adjusted for specific compound; Flow rate: 20 mL/min; Column temperature: ambient temperature; UV Wavelength: 214 and 254 nm. Analytical LCMC were collected using one of following methods—

### Method 1

Equipment: Shimadzu LCMS 2020 Mass Spectrometer; Column: HALO C₁₈ 2.7 μm, 3.0 mm×30 mm; Mobile Phase: MeCN (0.05% HCOOH)-Water (0.05% HCOOH); Gradient: MeCN from 5% to 95% over 1.4 min, hold 0.6 min, total run time is 2.5 min; Flow rate: 1.8 mL/min; Column temperature: 50° C.; Wavelength: 214 and 254 nm PDA.

### Method 2

Equipment: Shimadzu LCMS 2020 Mass Spectrometer; XBridge BEH C₁₈ 2.5 μm, 3.0 mm×30 mm Mobile Phase: MeCN-Water (0.1% NH₄OH); Gradient: MeCN from 5% to 95% over 1.8 min, hold 0.7 min, total run time is 3.0 min; Flow rate: 1.0 mL/min; Column temperature: 50° C.; Wavelength: 214 and 254 nm PDA.

### Method 3

Equipment: Shimadzu LCMS 2020 Mass Spectrometer; Column: HALO C₁₈ 2.7 μm, 3.0 mm×30 mm Mobile Phase: MeCN (0.05% TFA)-Water (0.05% TFA); Gradient: MeCN from 5% to 95% over 1.4 min, hold 0.6 min, total run time is 2.5 min; Flow rate: 1.8 mL/min; Column temperature: 50° C.; Wavelength: 214 and 254 nm PDA.

### SFC Chiral Resolution was Performed on Shimadzu Nexera UC Preparative SFC System (SFE-30A, LC-30AD_{SF}, SFC-30A) Using Following Methods:

### Method 1

Column: Daicel chiralpak-AS-H 5 μm 250×20 mm; Mobile Phase: CO₂/MeOH [0.1% NH₃ (7M in MeOH)], CO₀₂/MeOH ratio varies for different compounds; Oven temperature: 40° C.; Flow rate: 38 mL/min.

### Method 2

Column: Daicel chiralpak-OJ-H 5 μm 250*20 mm; Mobile Phase: CO₂/MeOH (0.1% HCOOH), CO₀₂/MeOH ratio varies for different compounds; Oven temperature: 40° C.; Flow rate: 38 mL/min.

### Method 3

Column: Daicel chiralpak-OD-H 5 μm 250×20 mm; Mobile Phase: CO₀₂/MeOH, ratio varies for different compounds; Oven temperature: 40° C.; Flow rate: 38 mL/min.

### Method 4

Column: Daicel chiralpak-AD-H 5 μm 250×20 mm; Mobile Phase: CO₀₂/i-PrOH, ratio varies for different compounds; Oven temperature: 40° C.; Flow rate: 38 mL/min.

### Method 5

Column: Daicel chiralpak-IC 5 μm 250×20 mm; Mobile Phase: CO₀₂/EtOH, ratio varies for different compounds; Oven temperature: 40° C.; Flow rate: 38 mL/min.

Unless otherwise stated, ¹H nuclear magnetic resonance spectroscopy (NMR) spectra were recorded on a Bruker AVANCE NEO 400 MHz Digital NMR Spectrometer. Chemical shifts, 6, are quoted in parts per million (ppm) relative to TMS and calibrated using residual un-deuterated solvent as an internal reference. The following abbreviations are used to denote the multiplicities and general assignments: s (singlet), d (doublet), t (triplet), q (quartet), dd (doublet of doublets), ddd (doublet of doublet of doublets), dt (doublet of triplets), dq (doublet of quartets), hep (heptet), m (multiplet), pent (pentet), td (triplet of doublets), qd (quartet of doublets), app. (apparent) and br. (broad). Coupling constants, J, are quoted to the nearest 0.1 Hz.

### Abbreviations and Acronyms

When the following abbreviations are used herein, they have the following meaning:

| | |
|---|---|
| (Boc)₂O | di-tert-butyl dicarbonate |
| (COCl)₂ | oxalyl chloride |
| (Me₃Si)₂NH | hexamethyldisilane |
| (NH₄)₂CO₃ | ammonium carbonate |
| [Ph₂SCF₃]⁺[OTf]⁻ | Yagupolskii-Umemoto reagent |
| Ac2O | acetic anhydride |
| AcOH | acetic acid |
| AIBN | 2,2′-axo bisisobutyronitrile |
| anhy | Anhydrous |
| atm | Atmosphere |
| BH₃-THF | borane-tetrahydrofuran |
| br | broad |
| bs | broad singlet |
| CD₃OD | methanol-d₄ |
| CDCl₃ | chloroform-d |
| Celite ® | diatomaceous earth filter agent, ® Celite Corp. |
| CH₂Cl₂ | methylene chloride |
| CH₃CN | acetonitrile |
| CH₃COCl | acetyl chloride |
| CHCl₃ | chloroform |
| CI-MS | chemical ionization mass spectroscopy |
| CO | carbon monoxide |
| conc | concentrated |
| Cs₂CO₃ | cesium carbonate |
| CsCl | caesium chloride |
| CsF | caesium flouride |
| CsOH | caesium hydroxide |
| Cu | copper |
| CuCN | copper(I) cyanide |
| CuI | copper(I) iodide |
| DBU | 1,8-Diazabicyclo[5.4.0]undec-7-ene |
| DCM | dichloromethane |
| dec | decomposition |
| DIEA | diisopropylethylamine |
| DMA | dimethylacetamide |
| DMAP | N,N-dimethylpyridin-4-amine |
| DME | dimethoxyethane |
| DMF | N,N-dimethylformamide |
| DMSO | dimethylsulfoxide |
| DMSO-d₆ | dimethylsulfoxide-d₆ |
| DPPF | 1,1-bis(diphenylphosphino)ferrocene |
| EDC | N-(3-dimethylaminopropyl)-N′-ethylcarbodiimide hydrochloride |
| ee | Enantiomeric excess |
| EGTA | ethylene glycol-bis(β-aminoethyl ether)-N,N,N′,N′-tetraacetic |
| | acid |
| ELSD | evaporative light scattering device |
| Et₂O | diethyl ether |
| Et₃N | triethylamine |
| Et₃N | triethylamine |
| EtOAc | ethyl acetate |
| EtOAc | ethyl acetate |
| EtOH | ethanol (100%) |
| FA | Formic acid |
| Fe | iron |
| H₂O | water |
| H₂O₂ | hydrogen peroxide |
| H₂SO₄ | sulfuric acid |
| HATU | N-[(dimethylamino)-1H-1,2,3-triazolo-[4,5-b]pyridin-1- |
| | ylmethylene]-N-methylmethanaminium hexafluorophosphate N- |
| | oxide |
| HBr | hydrobromic acid |
| HCl | hydrochloric acid |
| HClO₄ | perchloric acid |
| HEPES | 4-(2-hydroxyethyl)-1-piperazineethanesulfonic acid |
| HI | hydrogen iodide |
| HMPA | hexamethylphosphoramide |
| HNO₃ | nitric acid |
| HPLC ESI-MS | high performance liquid chromatography-electrospray |
| K₂CO₃ | potassium carbonate |
| K₃PO₄ | potassium phosphate |
| KCl | potassium chloride |
| KH | potassium hydride |
| KI | potassium iodide |
| KOAc | potassium acetate |
| KOH | potassium hydroxide |
| LC-MS (ESI) | liquid chromatography-mass spectroscopy (electrospray |
| | ionization) |
| LDA | lithium diisopropylamide |
| LiOH | lithium hydroxide |
| m-CPBA | meta-chloroperoxybenzoic acid |
| Me₃Al | trimethylaluminum |
| MeCN | acetonitrile |
| MeI | iodomethane |
| MeNO₂ | nitromethane |
| MeOH | methanol |
| MgCl₂ | magnesium chloride |
| MgSO₄ | magnesium sulfate |
| MPLC | medium pressure liquid chromatography |
| ms | mass spectroscopy |
| MS | Mass spectra |
| MsCl | Methanesulfonylchloride |
| Na₂CO₃ | sodium carbonate |
| Na₂SO₄ | sodium sulfate |
| NaCl | sodium chloride |
| NaHCO₃ | sodium bicarbonate |
| NaI | sodium iodide |
| NaNO₂ | sodium nitrite |
| NaOAc | sodium acetate |
| NaOH | sodium hydroxide |
| NBS | N-Bromosuccinimide |
| n-BuLi | n-butyllithium solution |
| n-BuOH | n-butanol |
| NCCH₂CN | malononitrile |
| NCS | N-Chlorosuccinimide |
| NH₂CO₂NH₄ | ammonium carbamate |
| NH₃ | ammonia |
| NH₄Cl | ammonium chloride |
| NH₄OH | ammonium hydroxide |
| NIS | N-Iodosuccinimide |
| NMM | 4-methylmorpholine |
| NMP | N-Methyl-2-pyrrolidone |
| NMR | nuclear magnetic resonance spectroscopy |
| P(O)Cl₃ | phosphorous oxychloride |
| Pd(dppf)Cl₂ | [1,1′-bis(diphenylphosphino)ferrocene]dichloropalladium(II) |
| Pd(DTBPF)Cl₂ | [1,1′-bis(di-tert-butylphosphino)ferrocene]dichloropalladium(II) |
| Pd(OAc)₂ | palladium(II) acetate |
| Pd(OAc)₂ | palladium(II) acetate |
| Pd(PPh₃)₄ | tetrakis(triphenylphosphine)palladium(0) |
| Pd₂(dba)₃ | tris(dibenzylideneacetone)dipalladium(0) |
| PdCl₂(PPh₃)₂ | dichlorobis(triphenylphosphine)palladium(II) |
| PE | petroleum ether |
| PG | protecting group |
| Ph₃P | triphenylphosphine |
| Ph₃PCl₂ | p,p-dichlorophenylphosphine |
| PhI(OAc)₂ | (diacetoxyiodo)benzene |
| PhOPOCl₂ | phenyl dichlorophosphate |
| PIDA | (diacetoxyiodo)benzene |
| p-TsOH•H₂O | p-toluenesulfonic acid |
| Rf | TLC retention factor |
| Rt | retention time (HPLC) |
| rt | room temperature |
| Ru-Phos | 2-dicyclohexylphosphino-2′,6′-diisopropoxybiphenyl |
| SFC | supercritical fluid hromatography |
| SiO₂ | silica gel |
| SOCl₂ | thionyl chloride |
| S-Phos | 2-dicyclohexylphosphino-2′,6′-dimethoxybiphenyl |
| TBAI | tetra-n-butylammonium iodide |
| TBSCl | tert-butyldimethylsilyl chloride |
| TBSCl | tert-butyldimethylsilyl chloride |
| t-BuOH | t-butanol |
| tBuOK | potassium tert-butoxide |
| tBuXPhos-Pd-G₃ | [(2-di-tert-butylphosphino-2′,4′,6′-triisopropyl-1,1′-biphenyl)-2- |
| | (2′-amino-1,1′-biphenyl)] palladium(II) methanesulfonate |
| Tf₂O | trifluoromethanesulfonic anhydride |
| TFA | trifluoroacetic acid |
| TFE | tetrafluoroethylene |
| THF | tetrahydrofuran |
| TLC | thin layer chromatography |
| TMSI | trimethylsilyl iodide |
| TOF-MS | time-of-flight-mass spectroscopy |
| TsC1 | p-toluenesulfonyl chloride |
| XantPhos | 4,5-bis(diphenylphosphino)-9,9-dimethylxanthene |
| Xantphos-Pd-G2 | chloro[(4,5-bis(diphenylphosphino)-9,9-dimethylxanthene)-2-(2′- |
| | amino-1,1′-biphenyl)]palladium(II) |
| Zn | zinc |
| Zn(CN)₂ | zinc cyanide |

### General Synthetic Schemes

Several methods for preparing the compounds of this invention are illustrated in the following Schemes and Examples. The present invention further provides processes for the preparation of compounds of structural Formula I as defined above. In some cases, the order of carrying out the foregoing reaction schemes may be varied to facilitate the reaction or to avoid unwanted reaction products. The following examples are provided for the purpose of illustration only and are not to be construed as limitations on the disclosed invention.

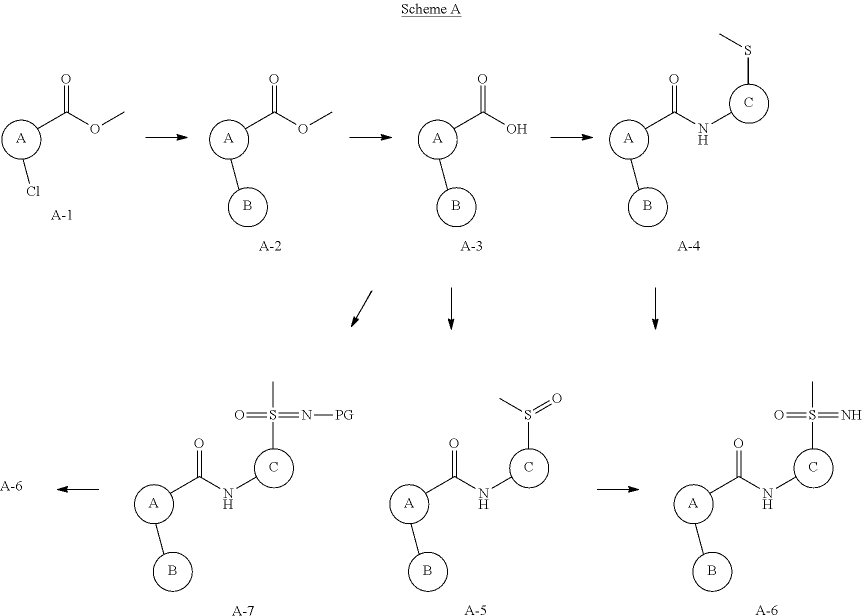

As illustrated in Scheme A, in general, compounds of the invention can be prepared by reacting substituted cyclic amines (B) using base such as DIEA or inorganic base such as K₂CO₃ or Cs₂CO₃ to afford intermediate A-2. Intermediate A-2 can be converted to corresponding acid A-3 by treating A-2 with base such as KOH in aqueous EtOH or MeOH. Intermediates A-4, A-5 and A-7 can be formed either by treating A-3 and amine C, utilizing amide coupling conditions or by activation of appropriately functionalized carboxylic acid A-3 with (COCl)₂ or POCl₃ and with amine C base such as DIEA or pyridine in DCM. Intermediate A-4 can be prepared directly by treating A-2 and amine C in presence of Me₃Al and toluene as a solvent. The compounds of formula A-4 and A-5 are independently treated with ammonium carbonate and PIDA in methanol to deliver compound of formula A-6. The compounds of formula A-6 can also be formed from intermediate A-7 by removing a protecting group, such as Boc under acidic conditions.

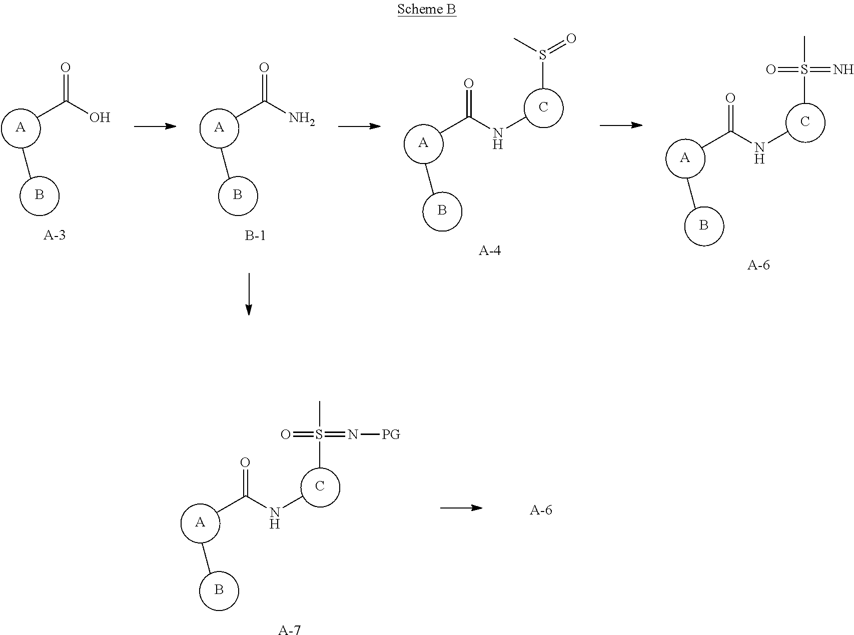

As illustrated in Scheme B, in general, compounds of the invention can be prepared by activation of appropriately functionalized carboxylic acid A-3 in dioxane with either (COCl)₂ or SOCl₂ followed by addition of NH₄OH to afford B-1. Intermediate B-1 can then be brought together with materials of variously substituted Br compounds, utilizing Xantphos-Pd-G2 mediated coupling conditions to deliver intermediate A-4 and A-7. The compounds of formula A-4 treated with ammonium carbonate and PIDA in methanol to deliver compound of formula A-6. The compounds of formula A-6 can also be formed from intermediate A-7 by removing a protecting group, such as Boc under acidic conditions.

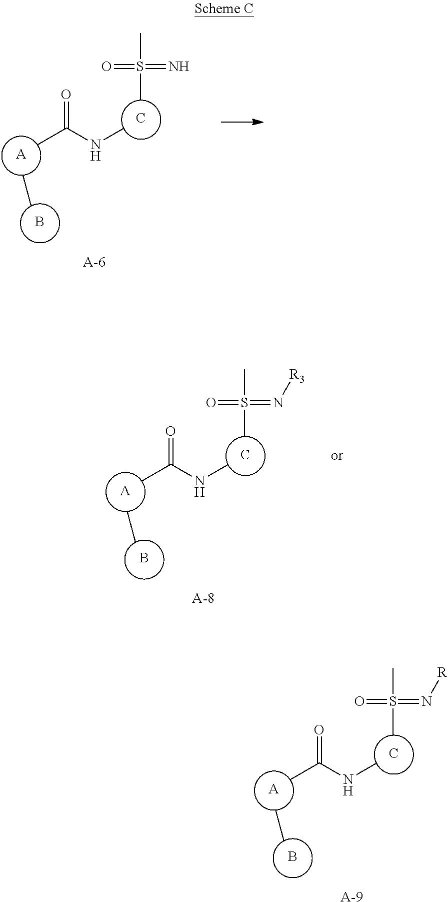

As further illustrated in Scheme C, in general, compounds of the invention can be prepared by derivatization of compounds of formula A-6 with R₃ or R′ substituents. Acylation of A-6 with an activated acid such as an acid chloride and TEA followed by removal of any protecting groups would provide compounds of formula A-8 or A-9. When A-8 or A-9 are an N-acylated sulfoximine, the carbonyl group may be reduced with borane to a methylene group (CH₂) to provide the corresponding N-alkyl derivative. Alternatively, N-alkyl derivatives may be obtained by alkylation with an alkyl halide such as, but not limited to, methyl iodide and a base such as diisopropylethylamineor Pd mediated coupling of A-6 with alkyl boronic acids.

### Specific Synthesis

### Example 1

### 3-(Azepan-1-yl)-N-(3-(S-methylsulfonimidoyl)phenyl)-6-(trifluoromethyl)pyridazine-4-carboxamide

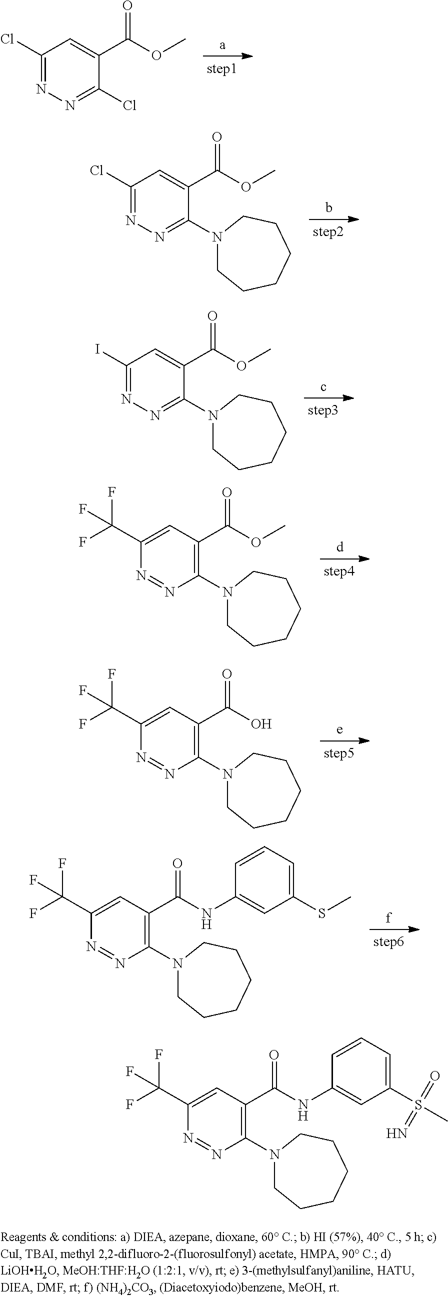

Step 1. methyl 3-(azepan-1-yl)-6-chloropyridazine-4-carboxylate: To a solution of methyl 3,6-dichloropyridazine-4-carboxylate (2.50 g, 12.1 mmol) was in dioxane (20 mL) was added azepane (1.26 g, 12.7 mmol) followed by DIEA (4.21 mL, 24.2 mmol) at rt. The mixture was stirred at rt for further 3 h, at the end of this period solvent was evaporated and the crude was chromatographed over SiO₂ with a gradient of 0-25% EtOAc in DCM to provide methyl 3-(azepan-1-yl)-6-chloropyridazine-4-carboxylate (2.86 g, 87.8%). ¹H NMR (300 MHz, CDCl₃) δ 7.40 (s, 1H), 4.03 (s, 1H), 3.92 (s, 3H), 3.58-3.48 (m, 5H), 1.86 (qd, J=6.2, 5.4, 2.4 Hz, 5H), 1.60-1.47 (m, 5H)

Step 2. methyl 3-(azepan-1-yl)-6-iodopyridazine-4-carboxylate: A mixture of methyl 3-(azepan-1-yl)-6-chloropyridazine-4-carboxylate (3.26 g, 12.1 mmol) and HI (25 mL, 57% v/v) was stirred 50 to 55° C. overnight, cooled to rt and the solid separated was filtered and washed with water (2×20 mL). The filtered cake was dissolved in EtOAc, dried over Na₂SO₄, filtered and the solvent evaporated under reduced pressure to give methyl 3-(azepan-1-yl)-6-iodopyridazine-4-carboxylate (2.16 g, 49.52%). LCMS: m/z 362.1[M+H]⁺

Step 3. methyl 3-(azepan-1-yl)-6-(trifluoromethyl)pyridazine-4-carboxylate: To a mixture of methyl 3-(azepan-1-yl)-6-iodopyridazine-4-carboxylate (2.16 g, 5.98 mmol) in HMPA (25 mL), CuI (1.62 g, 8.49 mmol) was added tetrabutylammoniumiodide (1.10 g, 2.99 mmol) under nitrogen, the reaction mixture was degassed for 5 min to the above mixture methyl 2,2-difluoro-2-(fluorosulfonyl) acetate(7.61 mL, 59.8 mmol) was added slowly and stirred at 90° C. for 1.5 h. The mixture was filtered and washed with EtOAc (3×100 mL), to the filtrate water (50 mL) was added and extracted with EtOAc (3×120 mL). The combined organic layer was washed with brine (50 mL), dried over Na₂SO₄ and filtered. The filtrate was evaporated, and the residue was chromatographed over SiO₂ eluting with a gradient of 0-20% EtOAc in Hexanes to afford methyl 3-(azepan-1-yl)-6-(trifluoromethyl)pyridazine-4-carboxylate (0.667 g, 37%). LCMS: m/z 304.1[M+H]⁺

Step 4: 3-(azepan-1-yl)-6-(trifluoromethyl)pyridazine-4-carboxylic acid: To a solution of methyl 3-(azepan-1-yl)-6-(trifluoromethyl)pyridazine-4-carboxylate in a mixture of MeOH:THF:H₂O (1:2:1, v/v) 20 mL was added LiOH·H₂O at rt. Then the mixture was stirred at 50° C. for 2 h, at the end of this period reaction mixture was cooled to rt and the solvent was evaporated. The solid material was suspended in water (10 mL) and pH was adjusted to 3 using 1N HCl solution. The mixture was extracted with EtOAc (3×50 mL), combined EtOAc was washed with brine (30 mL) and dried over Na₂SO₄, filtered and solvent evaporated to give 3-(azepan-1-yl)-6-(trifluoromethyl)pyridazine-4-carboxylic acid (0.489 g, 77%). 1HNMR (300 MHz, DMSO-d₆) δ 7.86 (s, 1H), 3.63 (t, J=5.6 Hz, 4H), 1.90-1.71 (m, 4H), 1.60-1.36 (m, 4H)

Step 5. 3-(azepan-1-yl)-N-[3-(methylsulfanyl)phenyl]-6-(trifluoromethyl)pyridazine-4-carboxamide: To a mixture of 3-(azepan-1-yl)-6-(trifluoromethyl)pyridazine-4-carboxylic acid (0.100 g, 0.346 mmol), 3-(methylsulfanyl)aniline (0.0529 g, 0.380 mmol) and HATU (0.263 g, 0.691 mmol) in DMF(3 mL) was added DIEA (0.151 mL, 0.864 mmol) at rt and stirring continued further for 6 h. At the end of this period water(5 mL) was added ands extracted with EtOAc (2×25 mL), combined EtOAc layer was washed with 1M LiCl (20 mL) followed by brine(20 mL) and dried over Na₂SO₄, filtered and solvent evaporated under reduced pressure. The crude product was chromatographed over SiO₂ with a gradient of EtOAc in DCM to provide 3-(azepan-1-yl)-N-[3-(methylsulfanyl)phenyl]-6-(trifluoromethyl)pyridazine-4-carboxamide (0.128 g, 90%). LCMS: m/z 411.4[M+H]⁺

Step 6. 3-(azepan-1-yl)-N-{3-[imino(methyl)oxo-λ⁶-sulfanyl]phenyl}-6-(trifluoromethyl)pyridazine-4-carboxamide: To a solution of 3-(azepan-1-yl)-N-[3-(methylsulfanyl)phenyl]-6-(trifluoromethyl)pyridazine-4-carboxamide (0.110 g, 0.268 mmol) in MeOH (5 mL) was added (NH₄)₂CO₃ (0.0386 g, 0.402 mmol) and PIDA (0.199 g, 0.616 mmol) at rt. The mixture was stirred for 2 h at rt. The solvent was evaporated to dryness and the crude was chromatographed over SiO₂ with a gradient of 0-15 MeOH in DCM to provide 3-(azepan-1-yl)-N-{3-[imino(methyl)oxo-λ⁶-sulfanyl]phenyl}-6-(trifluoromethyl)pyridazine-4-carboxamide (15.4 g, 13%). ¹H NMR (300 MHz, CDCl₃) δ 8.83 (s, 1H), 8.22-8.02 (m, 2H), 7.81 (dt, J=8.0, 1.2 Hz, 1H), 7.70 (s, 1H), 7.60 (t, J=8.0 Hz, 1H), 3.68 (t, J=5.7 Hz, 4H), 3.12 (s, 3H), 1.85 (s, 4H), 1.54(bs, 4H). LC-MS: m/z 442.5[M+H]⁺

### Example 2

### 3-(azepan-1-yl)-5-methyl-N-(2-(S-methylsulfonimidoyl)pyridin-4-yl)-6-(trifluoromethyl)pyridazine-4-carboxamide

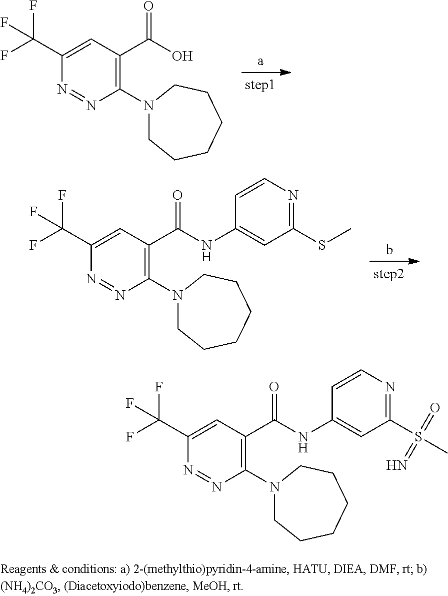

Step 1: 3-(azepan-1-yl)-N-[2-(methylsulfanyl)pyridin-4-yl]-6-(trifluoromethyl)pyridazine-4-carboxamide: To a mixture of 3-(azepan-1-yl)-6-(trifluoromethyl)pyridazine-4-carboxylic acid (0.100 g, 0.346 mmol), HATU(0.263 g, 0.691 mmol) and 2-(methylsulfanyl)pyridin-4-amine (0.0485 g, 0.346 mmol) in DMF(3 mL) was added DIEA(0.151 ml, 0.864 mmol) at rt. The mixture was stirred at rt for 16 h. At the end of this period water (5 mL) was added and extracted with EtOAc (2×25 mL), EtOAc layers were combined and washed with 1M LiCi (10 mL) followed by brine and dried (Na₂SO₄) filtered and the solvent evaporated to give crude product. The crude was chromatographed over SiO₂ with a gradient of 0-20% EtOAc in DCM to provide 3-(azepan-1-yl)-N-[2-(methylsulfanyl)pyridin-4-yl]-6-(trifluoromethyl)pyridazine-4-carboxamide (0.030 g, 21%). LC-MS: m/z 412.2[M+H]⁺

Step 2: 3-(azepan-1-yl)-N-{2-[imino(methyl)oxo-λ⁶-sulfanyl]pyridin-4-yl}-6-(trifluoromethyl)pyridazine-4-carboxamide: To a solution of 3-(azepan-1-yl)-N-[2-(methylsulfanyl)pyridin-4-yl]-6-(trifluoromethyl)pyridazine-4-carboxamide (0.026 g, 0.0632 mmol) in MeOH (5 mL) was added (NH₄)₄CO₃(0.0911 g, 0.0948 mmol) and PIDA (0.0468 g, 0.145 mmol) at rt. The mixture was stirred for 2 h at rt. The solvent was evaporated to dryness and the crude was chromatographed over SiO₂ with a gradient of 0-15 MeOH in DCM to provide 3-(azepan-1-yl)-N-{2-[imino(methyl)oxo-λ⁶-sulfanyl]pyridin-4-yl}-6-(trifluoromethyl)pyridazine-4-carboxamide (0.121 g, 43.3%) ¹H NMR (300 MHz, CDCl₃) δ 9.61 (s, 1H), 8.65 (d, J=5.5 Hz, 1H), 8.20 (dd, J=5.5, 2.0 Hz, 1H), 8.08 (d, J=2.0 Hz, 1H), 7.74 (s, 1H), 3.63 (q, J=4.8, 4.3 Hz, 4H), 3.26 (s, 3H), 1.85 (bs, 4H), 1.54(bs, 4H). LC-MS: m/z 443.5[M+H]⁺

### Example 3

### 2-(4,4-difluoroazepan-1-yl)-6-methyl-N-(2-sulfamoylpyridin-4-yl)-5-(trif luoromethyl)nicotinamide

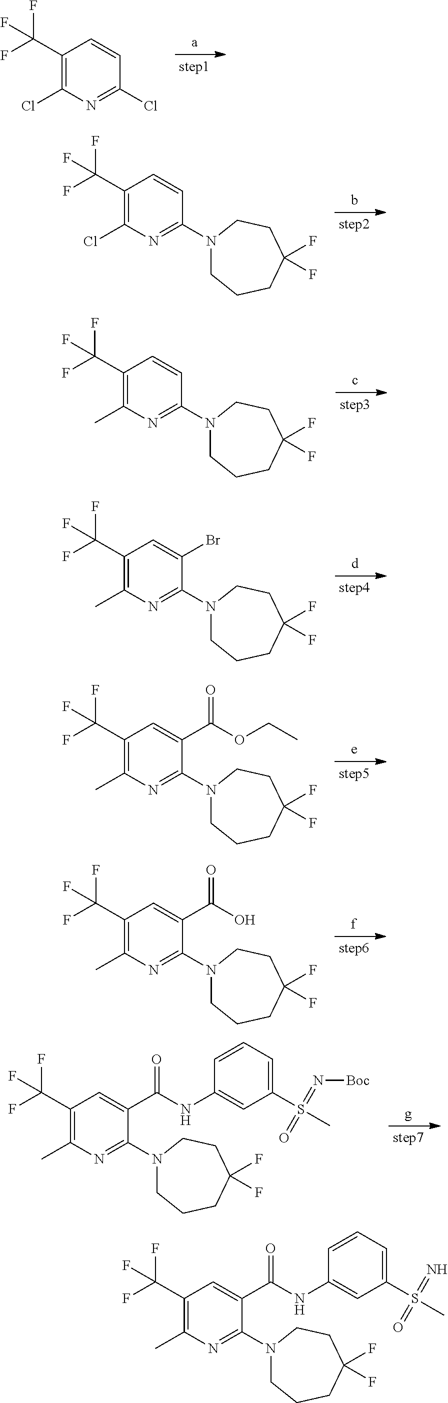

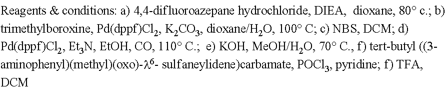

Step 1: 1-(6-chloro-5-(trifluoromethyl)pyridin-2-yl)-4,4-difluoroazepane: A mixture of 2,6-dichloro-3-(trifluoromethyl)pyridine (1.0 g, 4.6 mmol), 4,4-difluoroazepane hydrochloride (0.95 g, 5.5 mmol) and DIEA (1.19 g, 9.2 mmol) in dioxane (20 mL) was heated at 80° C. for 5 hours. Then the mixture was concentrated and residue was purified by flash column chromatography on silica gel (PE/EtOAc=5/1) to give 1-(6-chloro-5-(trifluoromethyl)pyridin-2-yl)-4,4-difluoroazepane (900 mg, 62.5%). LCMS (ESI) calcd. for C₁₂H₁₃ClF₅N₂ [M+H]⁺ m/z 315.07, found 315.05.

Step 2: 4,4-difluoro-1-(6-methyl-5-(trifluoromethyl)pyridin-2-yl)azepane: A mixture of 1-(6-chloro-5-(trifluoromethyl)pyridin-2-yl)-4,4-difluoroazepane (850 mg, 2.7 mmol), trimethylboroxine (3.39 g, 27 mmol), Pd(dppf)Cl₂ (98.73 mg, 0.135 mmol) and K₂CO₃ (1.12 g, 8.1 mmol) in dioxane/H₂O (4/1, 25 mL) was heated at 100° C. for 16 hours under an atmosphere of N₂. After cooling to ambient temperature, the mixture was filtered through celite, and the filtrate was concentrated under vacuum. The residue was diluted with water and extracted with EtOAc. The combined organic phases were washed with water and brine, dried over sodium sulfate, concentrated under vacuum. The residue was purified by flash column chromatography on silica gel (PE/EtOAc=5/1) to give the 4,4-difluoro-1-(6-methyl-5-(trifluoromethyl)pyridin-2-yl)azepane (800 mg, 91%). LCMS (ESI) calcd. for C₁₃H₁₆F5N₂ [M+H]⁺ m/z 295.13, found 295.00.

Step 3: 1-(3-bromo-6-methyl-5-(trifluoromethyl)pyridin-2-yl)-4,4-difluoroazepane: To a solution of 4,4-difluoro-1-(6-methyl-5-(trifluoromethyl)pyridin-2-yl)azepane (700 mg, 2.38 mmol) in DCM (15 mL) was added NBS (508 mg, 2.85 mmol). The mixture was stirred at room temperature for 2 hours. The resulting mixture was concentrated and directly purified by flash column chromatography on silica gel (PE/EtOAc=10/1) to give the 1-(3-bromo-6-methyl-5-(trifluoromethyl)pyridin-2-yl)-4,4-difluoroazepane (650 mg, 65.9%). LCMS (ESI) calcd. for C₁₃H₁₅BrF₅N₂ [M+H]⁺ m/z 373.04, found 372.95.

Step 4: Ethyl 2-(4,4-difluoroazepan-1-yl)-6-methyl-5-(trifluoromethyl)nicotinate: A mixture of 1-(3-bromo-6-methyl-5-(trifluoromethyl)pyridin-2-yl)-4,4-difluoroazepane (600 mg, 1.6 mmol), Pd(dppf)Cl₂ (58.5 mg, 0.08 mmol) and triethylamine (485 mg, 4.8 mmol) in EtOH (10 mL) was heated at 110° C. under an atmosphere of CO (30 atm) for 16 hours in a high-pressure reactor. After cooling to ambient temperature, the mixture was filtered through celite and the filtrate was concentrated under vacuum. The residue was purified by flash column chromatography on silica gel (PE/EtOAc=10/1) to give ethyl 2-(4,4-difluoroazepan-1-yl)-6-methyl-5-(trifluoromethyl)nicotinate (550 mg, 94%). LCMS (ESI) calcd. for C₁₆H₂₀F₅N₂O₂ [M+H]⁺ m/z 367.15, found 367.00.

Step 5: 2-(4,4-difluoroazepan-1-yl)-6-methyl-5-(trifluoromethyl)nicotinic acid: To a solution of ethyl 2-(4,4-difluoroazepan-1-yl)-6-methyl-5-(trifluoromethyl)nicotinate (550 mg, 1.5 mmol) in MeOH/H₂O (1/1, 20 mL) was added KOH (0.84 g, 15 mmol) at room temperature. The mixture was heated at 70° C. for 4 hours. After the reaction was completed, the mixture was cooled to room temperature and concentrated to remove MeOH. The aqueous phase was adjusted to pH=3-4 with 1 N HCl and extracted with EtOAc (50 mL×3). The combined organic phases were washed with brine, dried with Na₂SO₄, and concentrated under reduced pressure to give 2-(4,4-difluoroazepan-1-yl)-6-methyl-5-(trifluoromethyl)nicotinic acid (420 mg, 83%). LCMS (ESI) calcd. for C₁₄H₁₆F₅N₂O₂ [M+H]⁺ m/z 339.12, found 338.95.

Step 6: tert-butyl ((3-(2-(4,4-difluoroazepan-1-yl)-6-methyl-5-(trifluoromethyl)nicotinamido)phenyl)(methyl)(oxo)-λ⁶-sulfaneylidene)carbamate: To a solution of 2-(4,4-difluoroazepan-1-yl)-6-methyl-5-(trifluoromethyl)nicotinic acid (120 mg, 0.35 mmol) and tert-butyl ((3-aminophenyl)(methyl)(oxo)-λ⁶-sulfaneylidene)carbamate Int-3 (115 mg, 0.43 mmol) in pyridine (6 mL) was added POCl₃ (0.3 mL) dropwise at 0° C. The mixture was stirred at room temperature for 2 hours. LCMS showed the reaction was completed. The mixture was quenched with water (60 mL) and extracted with EtOAc (40 mL×3). The combine organic phases were washed with brine, dried over sodium sulfate, concentrated under vacuum. The residue was purified by flash column chromatography on silica gel (PE/EtOAc=1/2) to provide tert-butyl ((3-(2-(4,4-difluoroazepan-1-yl)-6-methyl-5-(trifluoromethyl)nicotinamido)phenyl)(methyl)(oxo)-λ⁶-sulfaneylidene)carbamate (57 mg, 24.4% o) as a yellow oil. LCMS (ESI) calcd. for C₂₆H₃₂F₅N₄O₄S [M+H]⁺ m/z 591.21, found 591.10.

Step 7: 2-(4,4-difluoroazepan-1-yl)-6-methyl-N-(3-(S-methylsulfonimidoyl)phenyl)-5-(trifluoromethyl)nicotinamide: To a solution of tert-butyl ((3-(2-(4,4-difluoroazepan-1-yl)-6-methyl-5-(trifluoromethyl)nicotinamido)phenyl)(methyl)(oxo)-λ⁶-sulfaneylidene)carbamate (57 mg, 0.10 mmol) in DCM (3 mL) was added TFA (1 mL) at room temperature. The mixture was stirred at room temperature for 2 hours. LCMS showed the reaction was completed. The final mixture was concentrated in vacuum and purified by prep-HPLC (Gemini 5 um C₁₈ column, 150×21.2 mm, eluting with 50% to 80% MeCN/H₂O containing 0.1% formic acid) to afford 2-(4,4-difluoroazepan-1-yl)-6-methyl-N-(3-(S-methylsulfonimidoyl)phenyl)-5-(trifluoromethyl) nicotinamide (11.3 mg, 23.2%) as a white solid. LCMS (ESI) calcd. for C₂₁H₂₄F₅N₄O₂S [M+H]⁺ m/z 491.15, found 491.05. ¹H NMR (400 MHz, DMSO-d₆) δ 10.81 (s, 1H), 8.31 (s, 1H), 8.04-7.85 (m, 2H), 7.67 (d, J=7.8 Hz, 1H), 7.60 (t, J=7.9 Hz, 1H), 3.71 (s, 2H), 3.59-3.33 (m, 5H), 3.13 (s, 3H), 2.40-2.67 (m, 2H), 2.03-1.83 (m, 4H).

### Example 4

### (R)-2-(4,4-difluoroazepan-1-yl)-6-methyl-N-(3-(S-methylsulfonimidoyl)phenyl)-5-(trifluoromethyl)nicotinamide

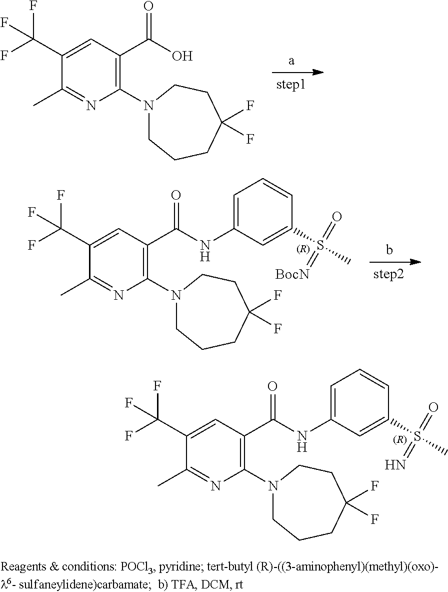

Step 1: tert-butyl (R)-((3-(2-(4,4-difluoroazepan-1-yl)-6-methyl-5-(trifluoromethyl)nicotinamido)phenyl)(methyl)(oxo)-λ⁶-sulfaneylidene)carbamate: A mixture of 2-(4,4-difluoroazepan-1-yl)-6-methyl-5-(trifluoromethyl)nicotinic acid (250 mg, 0.740 mmol) and tert-butyl (R)-((3-aminophenyl)(methyl)(oxo)λ⁶-sulfaneylidene)carbamate (220 mg, 0.814 mmol) in pyridine (4 mL) was added POCl₃ (375 L) dropwise at 50° C. The reaction was monitored by LCMS. After the reaction was completed, the mixture was cooled to room temperature. The resulting solution was diluted with water (15 mL) and extracted with DCM (15 mL×3). The combined organic phases were washed with brine, dried over Na₂SO₄, concentrated under vacuum. The residue was purified by flash column chromatography on silica gel (PE/EtOAc=1/1) to give tert-butyl (R)-((3-(2-(4,4-difluoroazepan-1-yl)-6-methyl-5-(trifluoromethyl)nicotinamido)phenyl)(methyl)(oxo)-λ⁶-sulfaneylidene)carbamate (120 mg, 27.5%) as a yellow oil. LCMS (ESI) calcd. for C₂₆H₃₂F₅N₄O₄S [M+H]⁺ m/z 591.21, found 591.15.

Step 2: (R)-2-(4,4-difluoroazepan-1-yl)-6-methyl-N-(3-(S-methylsulfonimidoyl)phenyl)-5-(trifluoromethyl)nicotinamide: A solution of tert-butyl (R)-((3-(2-(4,4-difluoroazepan-1-yl)-6-methyl-5-(trifluoromethyl)nicotinamido)phenyl)(methyl)(oxo)-λ⁶-sulfaneylidene)carbamate (120 mg, 0.200 mmol) in DCM (3 mL) was added TFA (1 mL) at room temperature. The reaction mixture was stirred at room temperature for 2 hours. After the reaction was completed, the filtrate was diluted with water (5 mL) and extracted with DCM (5 mL×3). The combined organic phases were washed with brine, dried over sodium sulfate, concentrated under vacuum. The residue was purified by prep-HPLC (Gemini 5 um C₁₈ column, 150*21.2 mm, eluting with 30% to 90% ACN-H₂O containing 0.1% formic acid) to obtain (R)-2-(4,4-difluoroazepan-1-yl)-6-methyl-N-(3-(S-methylsulfonimidoyl)phenyl)-5-(trifluoromethyl)nicotinamide (55 mg, 56.12%) as a white solid. ¹H NMR (400 MHz, DMSO-d₆) δ 10.78 (s, 1H), 8.29 (s, 1H), 7.94-7.82 (m, 2H), 7.65 (d, J=8 Hz, 1H), 7.59-7.55 (m, 1H), 4.20 (s, 1H), 3.49-3.47 (m, 2H), 3.33-3.31 (m, 2H), 3.05 (s, 3H), 2.33 (s, 3H), 1.96-1.92 (m, 2H), 1.89-1.86 (m, 4H). LCMS (ESI) calcd. for C₂₁H₂₄F₅N₄O₂S [M+H]⁺ m/z 491.16, found 490.95.

### Example 5

### (S)-2-(4,4-Difluoroazepan-1-yl)-6-methyl-N-(3-(S-methylsulfonimidoyl)phenyl)-5-(trifluoromethyl)nicotinamide

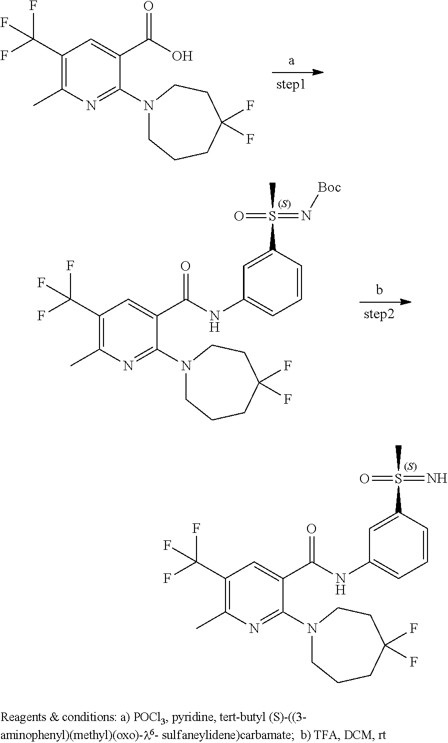

Step 1: tert-butyl (S)-((3-(2-(4,4-difluoroazepan-1-yl)-6-methyl-5-(trifluoromethyl)nicotinamido)phenyl)(methyl)(oxo)-λ⁶-sulfaneylidene)carbamate: A mixture of 2-(4,4-difluoroazepan-1-yl)-6-methyl-5-(trifluoromethyl)nicotinic acid (250 mg, 0.644 mmol) and tert-butyl (S)-((3-aminophenyl)(methyl)(oxo)-λ⁶-sulfaneylidene)carbamate (208.76 mg, 0.773 mmol) in pyridine (4 mL) was added POCl₃ (500 L) dropwise at 50° C. The reaction was monitored by LCMS. After the reaction was completed, the mixture was cooled to room temperature. The resulting solution was diluted with water (20 mL) and extracted with DCM (20 mL×3). The combined organic phases were washed with brine, dried over sodium sulfate, concentrated under vacuum. The residue was purified by flash column chromatography on silica gel (PE/EtOAc=1/1) to give tert-butyl (S)-((3-(2-(4,4-difluoroazepan-1-yl)-6-methyl-5-(trifluoromethyl)nicotinamido)phenyl)(methyl)(oxo)-λ⁶-sulfaneylidene)carbamate (130 mg, 34.21%) as a white oil. LCMS (ESI) calcd. for C₂₆H₃₂F₅N₄O₄S [M+H]⁺ m/z 591.21, found 591.40.

Step 2: (S)-2-(4,4-difluoroazepan-1-yl)-6-methyl-N-(3-(S-methylsulfonimidoyl)phenyl)-5-(trifluoromethyl)nicotinamide: A solution of tert-butyl (S)-((3-(2-(4,4-difluoroazepan-1-yl)-6-methyl-5-(trifluoromethyl)nicotinamido)phenyl)(methyl)(oxo)-λ⁶-sulfaneylidene)carbamate (130 mg, 0.22 mmol) in DCM (3 mL) was added TFA (1 mL) at room temperature. The reaction mixture was stirred at room temperature for 2 hours. The reaction was monitored by LCMS. After the reaction was completed, the mixture was diluted with water (10 mL) and extracted with DCM (10 mL×3). The combined organic phases were washed with brine, dried over sodium sulfate, concentrated under vacuum. The residue was further purified by prep-HPLC (Gemini 5 um C₁₈ column, 150*21.2 mm, eluting with 30% to 90% ACN-H₂O containing 0.1% formic acid) to obtain (S)-2-(4,4-difluoroazepan-1-yl)-6-methyl-N-(3-(S-methylsulfonimidoyl)phenyl)-5-(trifluoromethyl)nicotinamide (62 mg, 57.51%) as a white solid. ¹H NMR (400 MHz, DMSO-d₆) δ 10.79 (s, 1H), 8.29 (s, 1H), 7.95 (d, J=10.5 Hz, 2H), 7.70-7.52 (m, 2H), 4.27 (bs, 1H), 3.71 (s, 2H), 3.47 (t, J=5.5 Hz, 2H), 3.07 (s, 3H), 2.52 (s, 3H), 2.33 (s, 2H), 2.04-1.81 (m, 4H). LCMS (ESI) calcd. for C₂₁H₂₄F₅N₄O₂S [M+H]⁺ m/z 491.16, found 491.20.

### Example 6

### 3-(4,4-difluoroazepan-1-yl)-N-(3-(S-methylsulfonimidoyl)phenyl)-6-(trifluoromethyl)pyridazine-4-carboxamide

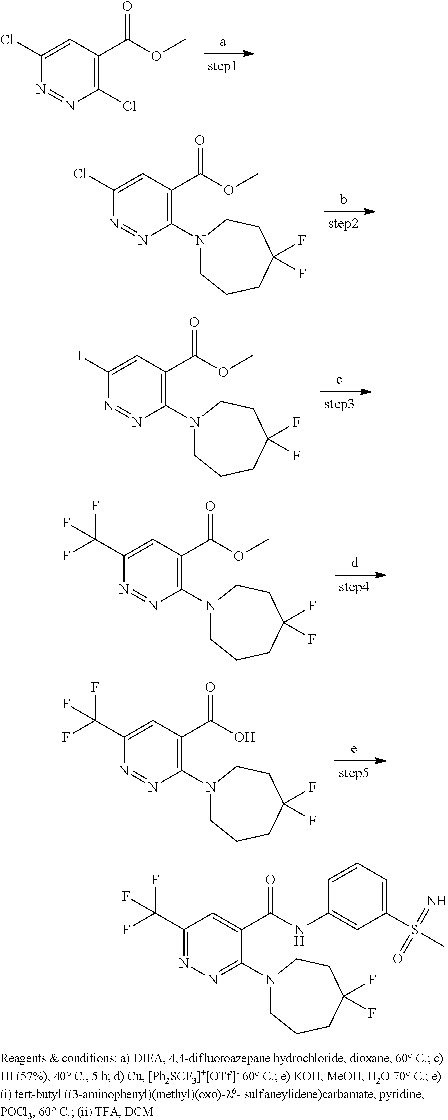

Step 1: methyl 6-chloro-3-(4,4-difluoroazepan-1-yl)pyridazine-4-carboxylate: To a solution of methyl 3,6-dichloropyridazine-4-carboxylate (2 g, 9.7 mmol) and 4,4-difluoroazepane hydrochloride (2.0 g, 11.6 mmol) in 1,4-dioxane (30 mL) was added DIEA (3.75 g, 29.1 mmol). The mixture was heated at 80° C. for 4 hours. LCMS showed the reaction was completed. The mixture was concentrated and purified by flash column chromatography on silica gel (PE/EtOAc=10/1) to give the methyl 6-chloro-3-(4,4-difluoroazepan-1-yl)pyridazine-4-carboxylate (1.6 g, 54.1%) as a yellow solid. LCMS (ESI) calcd. for C₁₂H₁₅ClF₂N₃O₂ [M+H]⁺ m/z 306.08, found 305.90.

Step 2: methyl 3-(4,4-difluoroazepan-1-yl)-6-iodopyridazine-4-carboxylate: A solution of methyl 6-chloro-3-(4,4-difluoroazepan-1-yl)pyridazine-4-carboxylate (1.6 g, 5.2 mmol) in HI (15 mL, 57% aqueous solution) was heated at 40° C. for overnight. After cooling to ambient temperature, the precipitate was collected by filtration, washed with water, and dried under vacuum to give the methyl 3-(4,4-difluoroazepan-1-yl)-6-iodopyridazine-4-carboxylate (1.8 g, 87.4%) as a yellow solid. LCMS (ESI) calcd. for C₁₂H₁₅IF₂N₃O₂ [M+H]⁺ m/z 398.02, found 397.85.

Step 3: methyl 3-(4,4-difluoroazepan-1-yl)-6-(trifluoromethyl)pyridazine-4-carboxylate. A solution of methyl 3-(4,4-difluoroazepan-1-yl)-6-iodopyridazine-4-carboxylate (1.8 g, 4.5 mmol), Cu (0.87 g, 13.6 mmol) and [Ph₂SCF₃]⁺[OTf]⁻ (3.6 g, 9.0 mmol) in DMF (20 mL) was heated at 60° C. for 5 hours. LCMS showed the reaction was completed. Then the mixture was filtered through celite. The filtrate was diluted with water and extracted with EtOAc (80 mL×3). The combine organic phases were washed with brine, dried over sodium sulfate, concentrated, and purified by silica gel column chromatography (eluting with EtOAc/PE=1/4) to give methyl 3-(4,4-difluoroazepan-1-yl)-6-(trifluoromethyl)pyridazine-4-carboxylate (1.2 g, 78.9%) as a yellow solid. LCMS (ESI) calcd. for C₁₂H₁₄F₃F₂N₃O₂ [M+H]⁺ m/z 340.11, found 339.95.

Step 4: 3-(4,4-difluoroazepan-1-yl)-6-(trifluoromethyl)pyridazine-4-carboxylic acid: To a solution of methyl 3-(4,4-difluoroazepan-1-yl)-6-(trifluoromethyl)pyridazine-4-carboxylate (800 mg, 2.35 mmol) in MeOH/H₂O (1/1, 20 mL) was added KOH (1.05 g, 18.8 mmol) at room temperature. The mixture was heated at 70° C. for 5 hours. After the reaction was completed, the mixture was cooled to room temperature and concentrated to remove MeOH. The aqueous phase was adjusted to pH=3-4 with 1N HCl and extracted with EtOAc (20 mL×3). The combined organic phases were washed with brine, dried with Na₂SO₄, and concentrated under reduced pressure to give 3-(4,4-difluoroazepan-1-yl)-6-(trifluoromethyl)pyridazine-4-carboxylic acid (620 mg, 81.2%) as a yellow solid. LCMS (ESI) calcd. for C₁₂H₁₃F₅N₃O₂ [M+H]⁺ m/z 326.09, found 325.95.

Step 5: To a solution of 3-(4,4-difluoroazepan-1-yl)-6-(trifluoromethyl)pyridazine-4-carboxylic acid (80 mg, 0.246 mmol) and tert-butyl ((3-aminophenyl)(methyl)(oxo)-λ⁶-sulfaneylidene)carbamate (66.5 mg, 0.246 mmol) in pyridine (2.5 mL) was added POCl₃ (20 uL) at 0° C. The mixture was stirred at 0° C. for 1 hour. LCMS showed the reaction was completed. The final mixture was quenched with water and extracted with EtOAc. The combined organic phases were washed with water and brine, dried with sodium sulfate, concentrated to give the crude intermediate. The crude intermediate was dissolved in DCM (3 mL) and added TFA (1 mL). The solution was stirred at room temperature for 1 hour then concentrated and purified by Prep-HPLC (Column: Gemini-C₁₈, 150×21.2 mm, 5 um; Mobile Phase: ACN-H₂O (0.1% FA), Gradient: 20%-38%-60%) to give the title compound (18.1 mg, 15.4% yield) as a white solid. ¹H NMR (400 MHz, DMSO-d₆) δ 11.07 (s, 1H), 8.26 (s, 1H), 8.08 (s, 1H), 7.94 (d, J=8.8 Hz, 1H), 7.71 (d, J=7.9 Hz, 1H), 7.62 (t, J=7.9 Hz, 1H), 4.25 (s, 1H), 3.87 (m, 2H), 3.58 (m, 2H), 3.06 (s, 3H), 2.36 (m, 2H), 2.12-1.86 (m, 4H). LCMS (ESI) calcd. for C₁₉H₂₁F₅N₅O₂S [M+H]⁺ m/z 478.13, found 478.05.

### Example 7

### 3-(azepan-1-yl)-5-methyl-N-(3-(S-methylsulfonimidoyl)phenyl)-6-(trifluoromethyl)pyridazine-4-carboxamide

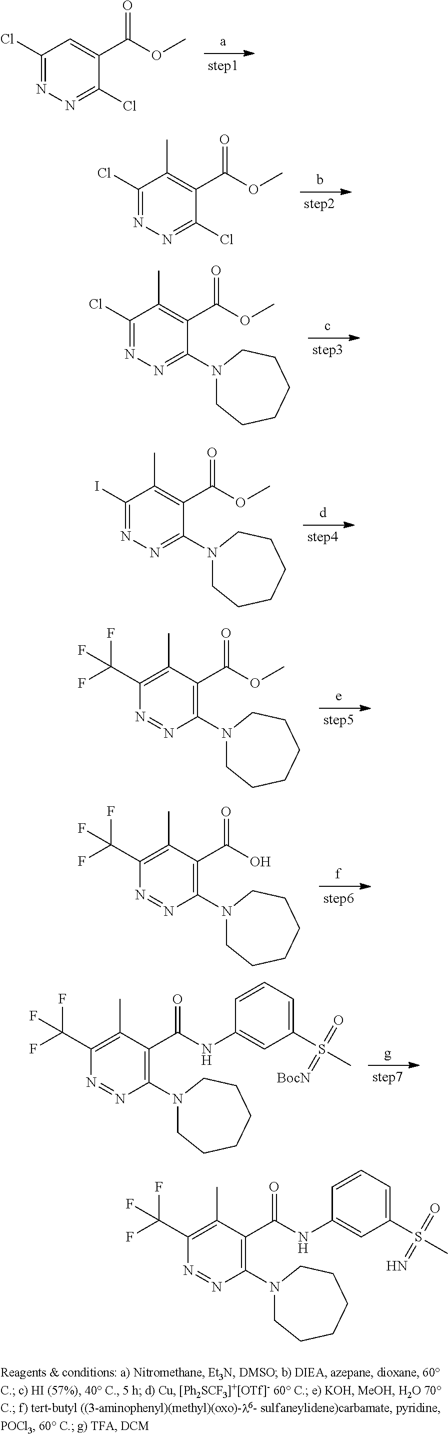

Step 1: methyl 3,6-dichloro-5-methylpyridazine-4-carboxylate: To a solution of methyl 3,6-dichloropyridazine-4-carboxylate (1 g, 4.86 mmol) in DMSO (10 mL) was added MeNO₂(1.48 g, 24.3 mmol). The mixture was stirred at room temperature for 30 min. Then TEA (0.74 g, 7.29 mmol) was added dropwise to the reaction at 0° C. The reaction was stirred at room temperature for 3 h. After the reaction was completed, the mixture was diluted with water (50 mL) and extracted with EtOAc (50 mL×3). The combine organic phases were washed with brine, dried over sodium sulfate, concentrated under vacuum. The residue was purified by flash column chromatography on silica gel (PE/EtOAc=10/1) to give methyl 3,6-dichloro-5-methylpyridazine-4-carboxylate (0.9 g, 84.3%) as a white solid. LCMS (ESI) calcd. for C₇H₇C₁₂N₂O₂ [M+H]⁺ m/z 220.99, found 220.85.

Step 2: methyl 3-(azepan-1-yl)-6-chloro-5-methylpyridazine-4-carboxylate: To a solution of methyl 3,6-dichloro-5-methylpyridazine-4-carboxylate (900 mg, 4.09 mmol) and azepane (484 mg, 4.89 mmol) in 1,4-dioxane (10 mL) was added DIEA (1583 mg, 12.27 mmol). The mixture was heated at 80° C. for 4 hours. LCMS showed the reaction was completed. The mixture was concentrated and purified by flash column chromatography on silica gel (PE/EtOAc=10/1) to give the methyl 3-(azepan-1-yl)-6-chloro-5-methylpyridazine-4-carboxylate (680 mg, 58.8%) as yellow solid. LCMS (ESI) calcd. for C₁₃H₁₉ClN₃O₂ [M+H]⁺ m/z 284.11, found 284.10.

Step 3: methyl 3-(azepan-1-yl)-6-iodo-5-methylpyridazine-4-carboxylate: A solution of methyl 3-(azepan-1-yl)-6-chloro-5-methylpyridazine-4-carboxylate (680 mg, 2.4 mmol) in HI (10 mL, 57% aqueous solution) was heated at 40° C. for overnight. After cooling to ambient temperature, the precipitate was collected by filtration, washed with water, and dried under vacuum to give the methyl 3-(azepan-1-yl)-6-iodo-5-methylpyridazine-4-carboxylate (500 mg, 55.5%) as a yellow solid. LCMS (ESI) calcd. for C₁₃H₁₉IN₃O₂ [M+H]⁺ m/z 376.05, found 376.10.

Step 4: methyl 3-(azepan-1-yl)-5-methyl-6-(trifluoromethyl)pyridazine-4-carboxylate: A solution of methyl 3-(azepan-1-yl)-6-iodo-5-methylpyridazine-4-carboxylate (500 mg, 1.33 mmol), Cu (257 mg, 4.02 mmol) and [Ph₂SCF₃]⁺[OTf]⁻ (1064 mg, 2.66 mmol) in DMF (10 mL) was heated at 60° C. for 5 hours. LCMS showed the reaction was completed. Then the mixture was filtered through celite. The filtrate was diluted with water (40 mL) and extracted with EtOAc (40 mL×3). The combine organic phases were washed with brine, dried over sodium sulfate, concentrated, and purified by silica gel column chromatography (eluting with EtOAc/PE=4/1) to give methyl 3-(azepan-1-yl)-5-methyl-6-(trifluoromethyl)pyridazine-4-carboxylate (350 mg, 82.7%) as a yellow solid. LCMS (ESI) calcd. for C₁₄H₁₉F₃N₃O₂ [M+H]⁺ m/z 318.14, found 318.05.

Step 5: 3-(azepan-1-yl)-5-methyl-6-(trifluoromethyl) pyridazine-4-carboxylic acid: To a solution of methyl 3-(azepan-1-yl)-5-methyl-6-(trifluoromethyl)pyridazine-4-carboxylate (350 mg, 1.1 mmol) in MeOH/H₂O (1/1, 8 mL) was added KOH (580 mg, 8.8 mmol) at room temperature. The mixture was heated at 70° C. for 5 hours. After the reaction was completed, the mixture was cooled to room temperature and concentrated to remove the MeOH. The aqueous phase was adjusted to pH=3-4 with 1N HCl and extracted with EtOAc (20 mL×3). The combined organic phases were washed with brine, dried with Na₂SO₄, and concentrated under reduced pressure to give 3-(azepan-1-yl)-5-methyl-6-(trifluoromethyl)pyridazine-4-carboxylic acid (250 mg, 74.8%) as a yellow solid. LCMS (ESI) calcd. for C₁₃H₁₇F₃N₃O₂ [M+H]⁺ m/z 304.12, found 304.05.

Step 6: tert-butyl ((3-(3-(azepan-1-yl)-5-methyl-6-(trifluoromethyl)pyridazine-4-carboxamido)phenyl)(methyl)(oxo)-λ⁶-sulfaneylidene)carbamate: To a solution of 3-(azepan-1-yl)-5-methyl-6-(trifluoromethyl)pyridazine-4-carboxylic acid (100 mg, 0.33 mmol) and tert-butyl ((3-aminophenyl)(methyl)(oxo)-λ⁶-sulfaneylidene)carbamate (89 mg, 0.33 mmol) in pyridine (4 mL) was added POCl₃ (30 uL) at room temperature. The mixture was heated at 60° C. for 1 hour. LCMS showed the reaction was completed. The final mixture was quenched with water and extracted with EtOAc. The combined organic phases were washed with water and brine, dried with sodium sulfate, concentrated, and purified by silica gel column chromatography (eluting with EtOAc/PE=1/2) to give tert-butyl((3-(3-(azepan-1-yl)-5-methyl-6-(trifluoromethyl) pyridazine-4-carboxamido) phenyl) (methyl)(oxo)-λ⁶-sulfaneylidene) carbamate (20 mg, 10.9%) as a yellow oil. LCMS (ESI) calcd. for C₂₅H₃₂F₃N₅O₄S [M+H]⁺ m/z 556.22, found [M+H]⁺ 556.15.

Step 7: 3-(azepan-1-yl)-5-methyl-N-(3-(S-methylsulfonimidoyl)phenyl)-6-(trifluoromethyl)pyridazine-4-carboxamide: To a solution of tert-butyl ((3-(3-(azepan-1-yl)-5-methyl-6-(trifluoromethyl)pyridazine-4-carboxamido)phenyl)(methyl)(oxo)-λ⁶-sulfaneylidene)carbamate (20 mg, 0.04 mmol) in DCM (2 mL) was added TFA (0.4 mL) at 0° C. The mixture was stirred room temperate for 2 hours. The mixture was concentrated and the residue was purified by Prep-HPLC (Column: Gemini-C18, 150×21.2 mm, 5 um; Mobile Phase: ACN-H₂O (0.1% FA), Gradient: 30%-40%-70%) to give 3-(azepan-1-yl)-5-methyl-N-(3-(S-methylsulfonimidoyl)phenyl)-6-(trifluoromethyl)pyridazine-4-carboxamide (5 mg, 30.5%) as a white solid. ¹H NMR (400 MHz, DMSO-d₆) δ 11.16 (s, 1H), 8.28 (s, 1H), 7.88 (d, J=7.9 Hz, 1H), 7.72 (d, J=8.1 Hz, 1H), 7.62 (t, J=7.9 Hz, 1H), 4.34 (s, 1H), 3.73 (t, J=5.9 Hz, 4H), 3.08 (s, 3H), 2.30 (s, 3H), 1.76 (s, 4H), 1.49 (s, 4H). LCMS (ESI) calcd. for C₂₀H₂₅F₃N₅O₂S [M+H]⁺ m/z 456.17, found 456.05.

### Example 8

### N-((3-aminophenyl)(methyl)(oxo)-λ⁶-sulfaneylidene)-3-(azepan-1-yl)-5-methyl-6-(trifluoromethyl)pyridazine-4-carboxamide

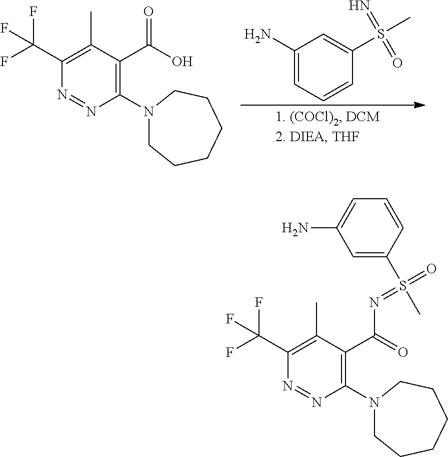 [SMILES: Cc1c(C(F)(F)F)nnc(N2CCCCCC2)c1C(=O)O ; CS(=N)(=O)c1cccc(N)c1 ; Cc1c(C(F)(F)F)nnc(N2CCCCCC2)c1C(=O)N=S(C)(=O)c1cccc(N)c1]

To a solution of 3-(azepan-1-yl)-5-methyl-6-(trifluoromethyl)pyridazine-4-carboxylic acid (100 mg, 0.33 mmol) in DCM (2 mL) was added oxalyl chloride (30 L) at room temperature. The mixture was stirred at room temperature for 30 minutes, then concentrated under reduced pressure. The resulting residue was dissolved in THE (1 mL) and added dropwise to a mixture of (3-aminophenyl)(imino)(methyl)-λ⁶-sulfanone (73 mg, 0.43 mmol) and DIEA (64 mg, 0.49 mmol) in THE (1 mL). The mixture was stirred at 40° C. for 2 h. LCMS showed the reaction was completed. The final mixture was quenched with water and extracted with EtOAc. The combined organic phases were washed with water and brine, dried with sodium sulfate and concentrated. The residue was purified by Prep-HPLC (Column: Gemini-C18, 150×21.2 mm, 5 um; Mobile Phase: ACN-H₂O (0.1% FA), Gradient: 20%-38%-60%) to give N-((3-aminophenyl)(methyl)(oxo)-λ⁶-sulfaneylidene)-3-(azepan-1-yl)-5-methyl-6-(trifluoromethyl)pyridazine-4-carboxamide (10 mg, 6.7%) as a white solid. ¹H NMR (400 MHz, DMSO-d₆) δ 7.31 (t, J=7.9 Hz, 1H), 7.17 (s, 1H), 7.10 (d, J=7.7 Hz, 1H), 6.88 (dd, J=8.1, 1.9 Hz, 1H), 5.79 (s, 2H), 3.75-3.62 (m, 4H), 3.60 (s, 3H), 2.27 (s, 3H), 1.74 (s, 4H), 1.56-1.37 (m, 4H). LCMS (ESI) calcd. for C₂₀H₂₅F₃N₅O₂S [M+H]⁺ m/z 456.17, found 456.00.

### Example 9

### 4-(azepan-1-yl)-N-(3-(S-methylsulfonimidoyl)phenyl)-6-(trifluoromethyl)pyridazine-3-carboxamide

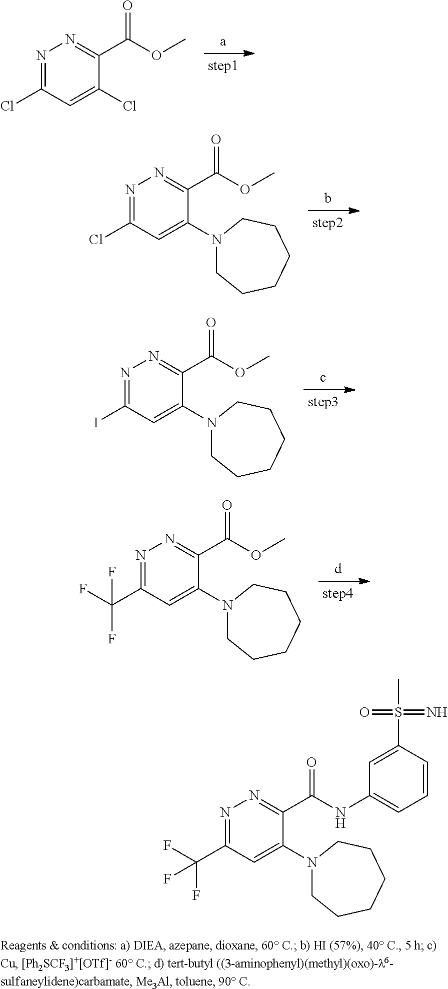

Step 1: methyl 4-(azepan-1-yl)-6-chloropyridazine-3-carboxylate: To a solution of methyl 4,6-dichloropyridazine-3-carboxylate (5 g, 24.3 mmol) and azepane (2.89 g, 29.2 mmol) in 1,4-dioxane (60 mL) was added DIEA (9.4 g, 72.9 mmol). The mixture was heated at 80° C. for 4 hours. LCMS showed the reaction was completed. The mixture was concentrated and purified by flash column chromatography on silica gel (PE/EtOAc=10/1) to give the methyl 4-(azepan-1-yl)-6-chloropyridazine-3-carboxylate (3.5 g, 53.8%) as a yellow solid. LCMS (ESI) calcd. for C₁₂H₁₇ClN₃O₂ [M+H]⁺ m/z 270.10, found 270.05.

Step 2: methyl 4-(azepan-1-yl)-6-iodopyridazine-3-carboxylate: A solution of methyl 4-(azepan-1-yl)-6-chloropyridazine-3-carboxylate (3.5 g, 11.8 mmol) in HI (30 mL, 57% aqueous solution) was stirred at 40° C. for overnight. After cooling to ambient temperature, the precipitate was collected by filtration, washed with water and dried under vacuum to give the methyl 4-(azepan-1-yl)-6-iodopyridazine-3-carboxylate (3.5 g, 82.2%) as a yellow solid. LCMS (ESI) calcd. for C₁₂H₁₇IN₃O₂ [M+H]⁺ m/z 362.03, found 362.00.

Step 3: methyl 4-(azepan-1-yl)-6-(trifluoromethyl)pyridazine-3-carboxylate: A solution of methyl 4-(azepan-1-yl)-6-iodopyridazine-3-carboxylate (2.5 g, 6.9 mmol), Cu (1.35 g, 20.7 mmol) and [Ph₂SCF₃]⁺[OTf]⁻ (5.5 g, 13.8 mmol) in DMF (30 mL) was heated at 60° C. for 5 hours. LCMS showed the reaction was completed. Then the mixture was filtered through celite. The filtrate was diluted with water and extracted with EtOAc (80 mL×3). The combined organic phases were washed with brine, dried over sodium sulfate, concentrated, and purified by silica gel column chromatography (eluting with EtOAc/PE=1/3) to give methyl 4-(azepan-1-yl)-6-(trifluoromethyl)pyridazine-3-carboxylate (1 g, 47.7%) as a yellow solid. LCMS (ESI) calcd. for C₁₃H₁₇F₃N₃O₂ [M+H]⁺ m/z 304.12, found 303.95.

Step 4: 4-(azepan-1-yl)-N-(3-(S-methylsulfonimidoyl)phenyl)-6-(trifluoromethyl)pyridazine-3-carboxamide. To a solution of tert-butyl ((3-aminophenyl)(methyl)(oxo)-λ⁶-sulfaneylidene)carbamate 534 mg, 1.98 mmol) in toluene (5 mL) was added Me₃Al (1M in hexane, 2.18 mL, 2.18 mmol) dropwise at 0° C. The mixture was stirred at 0° C. for 0.5 h. Then a solution of methyl 4-(azepan-1-yl)-6-(trifluoromethyl)pyridazine-3-carboxylate (200 mg, 0.66 mmol) in toluene (3 mL) was added at the same temperature. The resulting mixture was heated at 90° C. for 2 hours. LCMS showed the reaction was completed. The mixture was diluted with water (30 mL) and extracted with EtOAc (20 mL×3). The combined organic phases were washed with brine, dried with Na₂SO₄, concentrated, purified by prep-HPLC (Column: Gemini-C₁₈, 150×21.2 mm, 5 um; Mobile Phase: ACN-H₂O (0.1% FA), Gradient: 20%-38%-60%) to give 4-(azepan-1-yl)-N-(3-(S-methylsulfonimidoyl)phenyl)-6-(trifluoromethyl)pyridazine-3-carboxamide (60 mg, 20.6%) as a white solid. ¹H NMR (400 MHz, DMSO-d₆) δ 11.30 (s, 1H), 8.37 (s, 1H), 7.96 (d, J=8.7 Hz, 1H), 7.70 (d, J=7.9 Hz, 1H), 7.62 (t, J=7.9 Hz, 1H), 7.34 (s, 1H), 4.22 (s, 1H), 3.64-3.48 (m, 4H), 3.07 (s, 3H), 1.74 (s, 4H), 1.48 (s, 4H). LCMS (ESI) calcd. for C₁₉H₂₃F₃N₅O₂S [M+H]⁺ m/z 442.14, found 442.00.

### Example 10

### (R)-3-(azepan-1-yl)-5-methyl-N-(3-(S-methylsulfonimidoyl)phenyl)-6-(trifluoromethyl)pyridazine-4-carboxamide

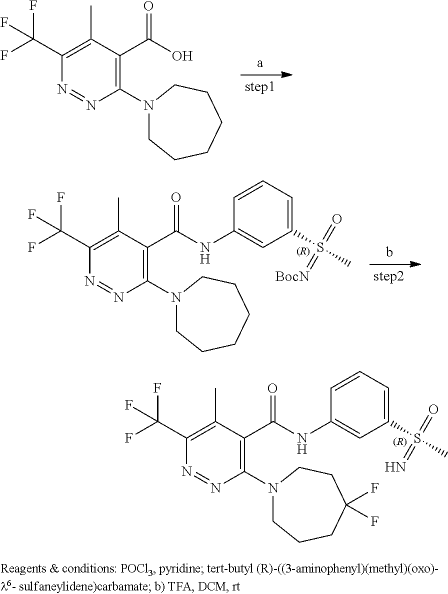

Step 1: tert-butyl (R)-((3-(3-(azepan-1-yl)-5-methyl-6-(trifluoromethyl)pyridazine-4-carboxamido)phenyl)(methyl)(oxo)-λ⁶-sulfaneylidene)carbamate: A mixture of 3-(azepan-1-yl)-5-methyl-6-(trifluoromethyl)pyridazine-4-carboxylic acid (100 mg, 0.330 mmol) and tert-butyl (R)-((3-aminophenyl)(methyl)(oxo)-16-sulfaneylidene)carbamate (133.70 mg, 0.495 mmol) in pyridine (2 mL) was added dropwise added POCl₃ (150 L) at 50° C. The reaction was monitored by LCMS. After the reaction was completed, the mixture was cooled to room temperature. The resulting solution was diluted with water (10 mL) and extracted with DCM (10 mL×3). The combine organic phases were washed with brine, dried over sodium sulfate, concentrated under vacuum. The residue was purified by flash column chromatography on silica gel (PE/EtOAc=1/1) to give tert-butyl (R)-((3-(3-(azepan-1-yl)-5-methyl-6-(trifluoromethyl)pyridazine-4-carboxamido)phenyl)(methyl)(oxo)-λ⁶-sulfaneylidene)carbamate (60 mg, 32.72%) as a yellow oil. LCMS (ESI) calcd. for C₂₅H₃₃F₃N₅O₄S [M+H]⁺ m/z 556.22, found 556.40.

Step 2: (R)-3-(azepan-1-yl)-5-methyl-N-(3-(S-methylsulfonimidoyl)phenyl)-6-(trifluoromethyl)pyridazine-4-carboxamide: A solution of tert-butyl (R)-((3-(3-(azepan-1-yl)-5-methyl-6-(trifluoromethyl)pyridazine-4-carboxamido)phenyl)(methyl)(oxo)-16-sulfaneylidene)carbamate (60 mg, 0.108 mmol) in DCM (1.5 mL) was added TFA (0.5 mL) at room temperature. The reaction mixture was stirred at room temperature for 2 hours. The reaction was monitored by LCMS. After the reaction was completed, the mixture was diluted with water (10 mL) and extracted with DCM (10 mL×3). The combine organic phases were washed with brine, dried over sodium sulfate, concentrated under vacuum. The residue was first purified by flash column chromatography on silica gel (PE/EtOAc=1/1), then further purified by prep-HPLC (Gemini 5 um C₁₈ column, 150×21.2 mm, eluting with 30% to 90% MeCN/H₂O containing 0.1% formic acid) to obtain (R)-3-(azepan-1-yl)-5-methyl-N-(3-(S-methylsulfonimidoyl)phenyl)-6-(trifluoromethyl)pyridazine-4-carboxamide (15 mg, 30%, 96.05% purity, 99% ee) as a white solid. ¹H NMR (400 MHz, DMSO-d₆) δ 11.16 (s, 1H), 8.27 (s, 1H), 7.92-7.85 (m, 1H), 7.72 (d, J=7.9 Hz, 1H), 7.62 (t, J=7.9 Hz, 1H), 4.26 (s, 1H), 3.73 (t, J=5.8 Hz, 4H), 3.07 (s, 3H), 2.30 (s, 3H), 1.76 (s, 4H), 1.49 (s, 4H). LCMS (ESI) calcd. for C₂₀H₂₃F₃N₅O₂S [M−H]⁻ m/z 454.15, found 454.10.

### Example 11

### (S)-3-(azepan-1-yl)-5-methyl-N-(3-(S-methylsulfonimidoyl)phenyl)-6-(trifluoromethyl)pyridazine-4-carboxamide

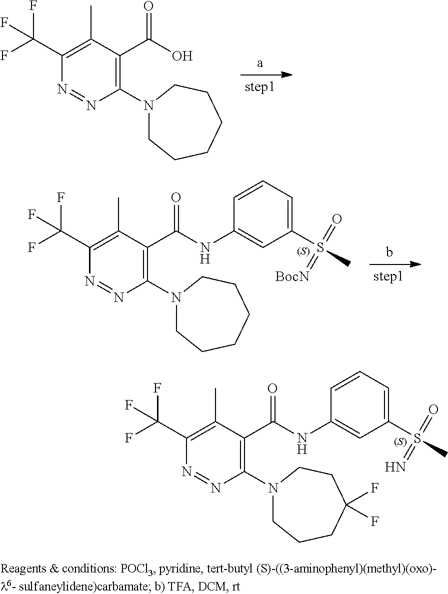

Step 1: tert-butyl (S)-((3-(3-(azepan-1-yl)-5-methyl-6-(trifluoromethyl)pyridazine-4-carboxamido)phenyl)(methyl)(oxo)-16-sulfaneylidene)carbamate: A mixture of 3-(azepan-1-yl)-5-methyl-6-(trifluoromethyl)pyridazine-4-carboxylic acid (100 mg, 0.330 mmol) and tert-butyl (S)-((3-aminophenyl)(methyl)(oxo)-16-sulfaneylidene)carbamate (133.70 mg, 0.495 mmol) in pyridine (2 mL) was added POCl₃ (150 μL) at 50° C. The reaction was monitored by LCMS.

After the reaction was completed, the mixture was cooled to room temperature. The resulting solution was diluted with water (10 mL) and extracted with DCM (10 mL×3). The combine organic phases were washed with brine, dried over Na₂SO₄, concentrated under vacuum. The residue was purified by flash column chromatography on silica gel (PE/EtAOc=1/1) to give tert-butyl (S)-((3-(3-(azepan-1-yl)-5-methyl-6-(trifluoromethyl)pyridazine-4-carboxamido)phenyl)(methyl)(oxo)-λ⁶-sulfaneylidene)carbamate (47 mg, 25.7%) as a yellow oil. LCMS (ESI) calcd. for C₂₅H₃₃F₃N₅O₄S [M+H]⁺ m/z 556.22, found 556.15.

Step 2: (S)-3-(azepan-1-yl)-5-methyl-N-(3-(S-methylsulfonimidoyl)phenyl)-6-(trifluoromethyl)pyridazine-4-carboxamide: A solution of tert-butyl (S)-((3-(3-(azepan-1-yl)-5-methyl-6-(trifluoromethyl)pyridazine-4-carboxamido)phenyl)(methyl)(oxo)-λ⁶-sulfaneylidene)carbamate (47 mg, 0.08 mmol) in DCM (1.5 mL) was added TFA (0.5 mL) at room temperature. The reaction mixture was stirred at room temperature for 2 hours. The reaction was monitored by LCMS. After the reaction was completed, the filtrate was diluted with water (10 mL) and extracted with DCM (10 mL×3). The combine organic phases were washed with brine, dried over sodium sulfate, concentrated under vacuum. The residue was first purified by flash column chromatography on silica gel (PE/EtOAc=1/1), then further purified by prep-HPLC (Gemini 5 um C₁₈ column, 150×21.2 mm, eluting with 30% to 90% MeCN/H₂O containing 0.1% formic acid) to obtain (S)-3-(azepan-1-yl)-5-methyl-N-(3-(S-methylsulfonimidoyl)phenyl)-6-(trifluoromethyl)pyridazine-4-carboxamide (18 mg, 49.45%, 98% purity, 100% ee) as a white solid. ¹H NMR (400 MHz, DMSO-d₆) δ 11.16 (s, 1H), 8.27 (s, 1H), 7.87 (d, J=8.4 Hz, 1H), 7.72 (d, J=7.6 Hz, 1H), 7.64-7.60 (m, 1H), 4.26 (s, 1H), 3.74-3.71 (m, 4H), 3.07 (s, 3H), 2.29 (s, 3H), 1.76 (s, 4H), 1.49 (s, 4H). LCMS (ESI) calcd. for C₂₀H₂₅F₃N₅O₂S [M+H]⁺ m/z 456.17, found 456.10.

### Example 12

### 5-chloro-6-methyl-N-(3-(S-methylsulfonimidoyl)phenyl)-2-(6-azaspiro[2.5]octan-6-yl)nicotinamide

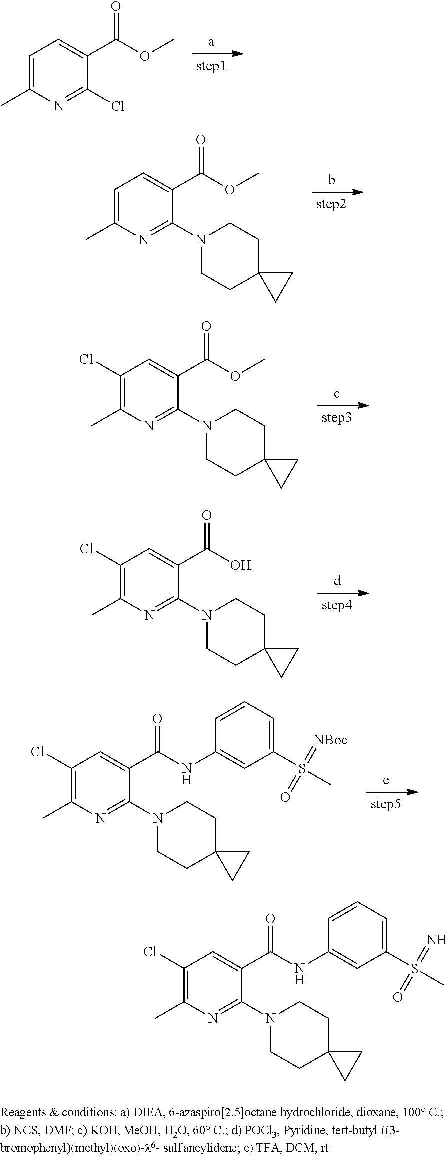

Step 1: methyl 6-methyl-2-(6-azaspiro[2.5]octan-6-yl)nicotinate: A mixture of methyl 2-chloro-6-methylnicotinate (1.3 g, 7 mmol) and 6-azaspiro[2.5]octane hydrochloride (1.03 g, 7 mmol) in 1,4-dioxane (20 mL) was added DIEA (1.82 g, 14 mmol) at room temperature. The reaction mixture was heated at 100° C. for 16 hours. After the reaction was completed, the mixture was cooled to room temperature, diluted with water (40 mL) and extracted with DCM (40 mL×3). The combine organic phases were washed with brine, dried over sodium sulfate, concentrated under vacuum. The residue was purified by flash column chromatography on silica gel (PE/EtOAc=3/1) to provide methyl 6-methyl-2-(6-azaspiro[2.5]octan-6-yl)nicotinate (1 g, 55.6%) as a white solid. LCMS (ESI) calcd. for C₁₅H₂₁N₂O₂ [M+H]⁺ m/z 261.16, found 260.95.

Step 2: methyl 5-chloro-6-methyl-2-(6-azaspiro[2.5]octan-6-yl)nicotinate: To a mixture of methyl 6-methyl-2-(6-azaspiro[2.5]octan-6-yl)nicotinate (0.950 g, 3.64 mmol) in DMF (10 mL) was added NCS (0.534 g, 4 mmol). The reaction mixture was stirred at room temperature for 8 hours. After the reaction was completed, the mixture was diluted with water (20 mL) and extracted with DCM (20 mL×3). The combine organic phases were washed with brine, dried over sodium sulfate, concentrated under vacuum. The residue was purified by flash column chromatography on silica gel (PE/EtOAc=3/1) to give methyl 5-chloro-6-methyl-2-(6-azaspiro[2.5]octan-6-yl)nicotinate (0.750 g, 70.0%) as a white oil. LCMS (ESI) calcd. for C₁₅H₂₀ClN₂O₂ [M+H]⁺ m/z 295.12, found 294.95.

Step 3: 5-chloro-6-methyl-2-(6-azaspiro[2.5]octan-6-yl)nicotinic acid: To a solution methyl 5-chloro-6-methyl-2-(6-azaspiro[2.5]octan-6-yl)nicotinate (0.700 g, 2.5 mmol) in MeOH/H₂O (1/1, 15 mL) was added KOH (0.561 g, 25 mmol) at room temperature. The mixture was heated at 60° C. for 6 hours. After the reaction was completed, the mixture was cooled to room temperature and extracted with DCM (20 mL). Then the aqueous phase was adjusted to pH=3 with 1N HCl and extracted with DCM (20 mL×3). The combined organic phases were dried with Na₂SO₄ and concentrated under reduced pressure to give 5-chloro-6-methyl-2-(6-azaspiro[2.5]octan-6-yl)nicotinic acid (560 mg, 79.6%) as a white solid. LCMS (ESI) calcd. for C₁₄H₁₈ClN₂O₂ [M+H]⁺ m/z 281.10, found 280.95.

Step 4: tert-butyl ((3-(5-chloro-6-methyl-2-(6-azaspiro[2.5]octan-6-yl)nicotinamido)phenyl)(methyl)(oxo)-λ⁶-sulfaneylidene)carbamate: To a mixture of 5-chloro-6-methyl-2-(6-azaspiro[2.5]octan-6-yl)nicotinic acid (500 mg, 1.78 mmol) and tert-butyl ((3-aminophenyl)(methyl)(oxo)-λ⁶-sulfaneylidene)carbamate (723 mg, 2.67 mmol) in pyridine (4 mL) at room temperature was added POCl₃ (750 L). The reaction mixture was stirred at room temperature for 2 hours. Then the mixture was diluted with water (10 mL) and extracted with DCM (10 mL×3). The combine organic phases were washed with brine, dried over sodium sulfate, concentrated under vacuum. The residue was purified by flash column chromatography on silica gel (PE/EtOAc=1/1) to give tert-butyl ((3-(5-chloro-6-methyl-2-(6-azaspiro[2.5]octan-6-yl)nicotinamido)phenyl)(methyl)(oxo)-λ⁶-sulfaneylidene)carbamate (120 mg, 12.64%) as a yellow oil. LCMS (ESI) calcd. for C₂₆H₃₄ClN₄O₄S [M+H]⁺ m/z 533.20, found 533.05.

Step 5: 5-chloro-6-methyl-N-(3-(S-methylsulfonimidoyl)phenyl)-2-(6-azaspiro[2.5]octan-6-yl)nicotinamide: A solution of tert-butyl ((3-(5-chloro-6-methyl-2-(6-azaspiro[2.5]octan-6-yl)nicotinamido)phenyl)(methyl)(oxo)-λ⁶-sulfaneylidene)carbamate (120 mg, 0.23 mmol) in DCM (3 mL) was added TFA (1 mL). The reaction mixture was stirred at room temperature for 2 hours. After the reaction was completed, the solution was diluted with water (10 mL) and extracted with DCM (10 mL×3). The combine organic phases were washed with brine, dried over sodium sulfate and concentrated under vacuum. The residue was first purified by flash column chromatography on silica gel (PE/EtOAc=1/1), then further purified by prep-HPLC (Gemini 5 um C₁₈ column, 150×21.2 mm, eluting with 30% to 90% MeCN/H₂O containing 0.1% formic acid) to afford the title compound (41 mg, 41.44%) as a white solid. ¹H NMR (400 MHz, DMSO-d₆) δ 10.81 (s, 1H), 8.39 (s, 1H), 7.88 (d, J=8.4 Hz, 1H), 7.80 (s, 1H), 7.65 (d, J=8 Hz, 1H), 7.60-7.56 (m, 1H), 4.21 (s, 1H), 3.36-3.32 (m, 4H), 3.05 (s, 3H), 2.46 (s, 3H), 1.38-1.35 (s, 4H), 0.28 (s, 4H). LCMS (ESI) calcd. for C₂₁H₂₆ClN₄O₂S [M+H]⁺ m/z 433.14, found 433.05.

### Example 13

### 6-(3-Buten-1-yl)-2-(4,4-difluoroazepan-1-yl)-N-(3-(S-methylsulfonimidoyl) phenyl)nicotinamide

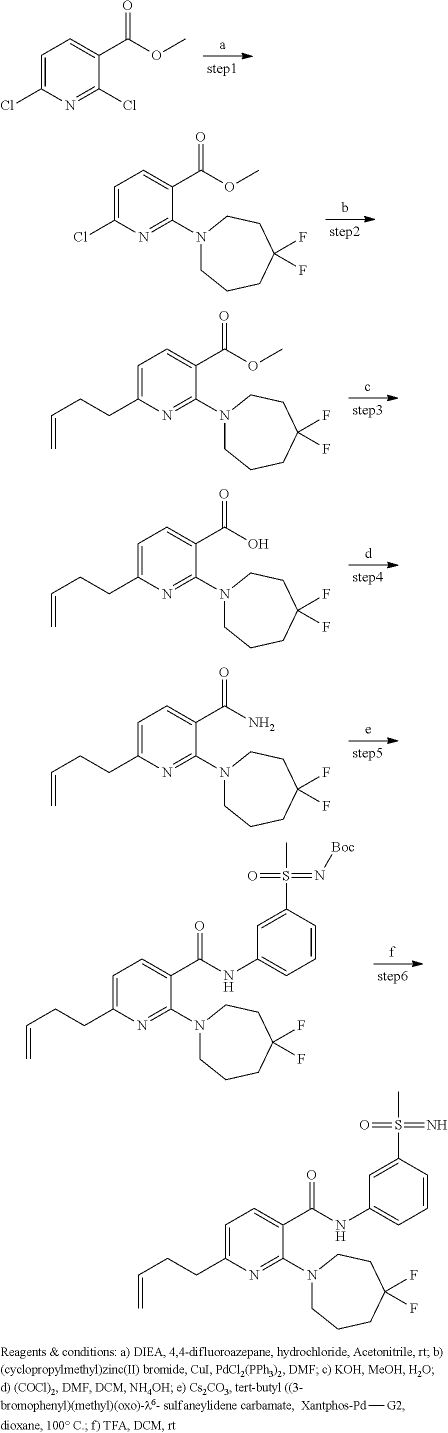

Step 1: methyl 6-chloro-2-(4,4-difluoroazepan-1-yl)nicotinate: A mixture of methyl 2,6-dichloronicotinate 1 (2.0 g, 9.7 mmol), 4,4-difluoroazepane hydrochloride (1.67 g, 9.7 mmol) and DIEA (3.75 g, 29.1 mmol) in dioxane (25 mL) was heated at 80° C. for 5 hours. Then the mixture was concentrated and directly purified by flash column chromatography on silica gel (PE/EtOAc=6/1) to give methyl 6-chloro-2-(4,4-difluoroazepan-1-yl)nicotinate (2.22 g, 67%). LCMS (ESI) calcd. for C₁₃H₁₆ClF₂N₂O₂ [M+H]⁺ m/z 305.09, found 305.90.

Step 2: methyl 6-(3-buten-1-yl)-2-(4,4-difluoroazepan-1-yl)nicotinate: A mixture of Zn power (2.2 g, 33.6 mmol) and 12 (850 mg, 3.36 mmol) in a 250 mL three-necked flask was evacuated and backfilled with N₂ three times and then charged with N₂. The flask was heated carefully with a hot air blower until 12 was sublimated. Then a solution of (bromomethyl)cyclopropane (2.27 g, 16.8 mmol) in DMF (8 mL) was added intermediately via a syringe. The mixture was stirred for 10 minutes while keeping the temperature was about 80° C. Then the heater and stirrer were removed. The upper layer clear solution was added dropwise into a stirred solution of methyl 6-chloro-2-(4,4-difluoroazepan-1-yl)nicotinate (1.7 g, 5.6 mmol), CuI (320 mg, 1.68 mmol) and Pd(pph₃)₂C₁₂ (390 mg, 0.56 mmol) in DMF (4 mL) under an atmosphere of N₂. The mixture was heated at 110° C. for 5 hours. Then the mixture was quenched with water (80 mL) and extracted with EtOAc (80 mL×3). The combined organic layers were washed with water and brine, dried over Na₂SO₄, filtered, concentrated to give the crude. The residue was purified by silica gel column chromatography (PE/EA, 4/1) to afford methyl 6-(3-buten-1-yl)-2-(4,4-difluoroazepan-1-yl)nicotinate (1.3 g, 64%) as a white solid. LCMS (ESI) calcd. for C₁₇H₂₃F₂N₂O₂ [M+H]⁺ m/z 325.17, found 325.25.

Step 3: 6-(3-buten-1-yl)-2-(4,4-difluoroazepan-1-yl)nicotinic acid: To a solution of methyl 6-(3-buten-1-yl)-2-(4,4-difluoroazepan-1-yl)nicotinate (400 mg, 1.23 mmol) in MeOH/H₂O (1/1, 8 mL) was added KOH (0.69 g, 12.3 mmol) at room temperature. The mixture was heated at 70° C. for 4 hours. After the reaction was completed, the mixture was cooled to room temperature and concentrated to remove MeOH. The aqueous phase was adjusted to pH=3-4 with 1N HCl and extracted with EtOAc (50 mL×3). The combined organic phases were washed with brine, dried with Na₂SO₄, and concentrated under reduced pressure to give 6-(3-buten-1-yl)-2-(4,4-difluoroazepan-1-yl)nicotinic acid (360 mg, 84.7%). LCMS (ESI) calcd. for C₁₆H₂₁F₂N₂O₂ [M+H]⁺ m/z 311.16, found 311.00.

Step 4: 6-(3-buten-1-yl)-2-(4,4-difluoroazepan-1-yl)nicotinamide: To a solution of 6-(3-buten-1-yl)-2-(4,4-difluoroazepan-1-yl)nicotinic acid (100 mg, 0.32 mmol) in DCM (5 mL) and DMF (10 L) was added oxalyl chloride (81.84 mg, 0.64 mmol) at 0° C. The mixture was stirred at 0° C. for 1 hour. After the reaction was completed, the mixture was concentrated to remove the solvent. Then the residue was dissolved in THE (5 mL) and was added to vigorously stirred NH₃—H₂O (2 mL). The mixture was stirred at room temperature for 1 hour. The resulting mixture was diluted with water and extracted with EtOAc. The combined organic phases were washed with water and brine, dried over sodium sulfate, and concentrated under vacuum to give crude 6-(3-buten-1-yl)-2-(4,4-difluoroazepan-1-yl)nicotinamide (96 mg, 86.7%) which was used in the next step without further purification. LCMS (ESI) calcd. for C₁₆H₂₂F₂N₃O [M+H]⁺ m/z 310.17, found 309.95.

Step 5: tert-butyl ((3-(6-(3-buten-1-yl)-2-(4,4-difluoroazepan-1-yl)nicotinamido)phenyl)(methyl)(oxo)-λ⁶-sulfaneylidene)carbamate: A solution of 6-(3-buten-1-yl)-2-(4,4-difluoroazepan-1-yl)nicotinamide 5 (90 mg, 0.29 mmol), tert-butyl ((3-bromophenyl)(methyl)(oxo)-λ⁶-sulfaneylidene)carbamate (195 mg, 0.58 mmol), Cs₂CO₃ (285 mg, 0.87 mmol) and Xantphos-Pd-G2 (26 mg, 0.029 mmol) in 1,4-dioxane (5 mL) was heated at 110° C. for overnight under an atmosphere of N₂. LCMS showed the reaction was completed. The mixture was filtered through celite and the filtrate was concentrated under vacuum. The residue was purified by flash column chromatography on silica gel (PE/EtOAc=3/1) to give tert-butyl ((3-(6-(3-buten-1-yl)-2-(4,4-difluoroazepan-1-yl)nicotinamido) phenyl)(methyl)(oxo)-λ⁶-sulfaneylidene)carbamate (50 mg, 27%) as a yellow oil. LCMS (ESI) calcd. for C₂₈H₃₇F₂N₄O₄S [M+H]⁺ m/z 563.25, found 563.20.

Step 6: 6-(3-buten-1-yl)-2-(4,4-difluoroazepan-1-yl)-N-(3-(S-methylsulfonimidoyl)phenyl)nicotinamide: To a solution of tert-butyl ((3-(6-(3-buten-1-yl)-2-(4,4-difluoroazepan-1-yl)nicotinamido)phenyl)(methyl)(oxo)-λ⁶-sulfaneylidene)carbamate (50 mg, 0.089 mmol) in DCM (3 mL) was added TFA (1 mL) at rt. The mixture was stirred room temperature for 2 hours. The resulting mixture was concentrated and the residue was purified by prep-HPLC (Column: Gemini-C18, 150×21.2 mm, 5 um; Mobile Phase: ACN-H₂O (0.1% FA), Gradient: 50%-80%) to give 6-(3-buten-1-yl)-2-(4,4-difluoroazepan-1-yl)-N-(3-(S-methylsulfonimidoyl) phenyl)nicotinamide (15 mg, 35%) as a white solid. ¹H NMR (400 MHz, DMSO-d₆) δ 10.65 (s, 1H), 8.34 (s, 1H), 7.92 (d, J=8.1 Hz, 1H), 7.63 (m, 2H), 7.56-7.61 (t, J=7.9 Hz, 1H), 6.66 (d, J=7.6 Hz, 1H), 5.93-5.81 (m, 1H), 5.08-4.94 (m, 2H), 4.19 (s, 1H), 3.63 (s, 2H), 3.42 (t, J=5.7 Hz, 2H), 3.05 (s, 3H), 2.73 (t, J=7.5 Hz, 2H), 2.48-2.42 (m, 2H), 2.39-2.26 (m, 2H), 2.01-1.80 (m, 4H). LCMS (ESI) calcd. for C₂₃H₂₉F₂N₄O₂S [M+H]⁺ m/z 463.20, found 463.10.

### Example 14

### N-(3-(S-methylsulfonimidoyl)phenyl)-3-(6-azaspiro[2.5]octan-6-yl)-6-(trifluoromethyl)pyridazine-4-carboxamide

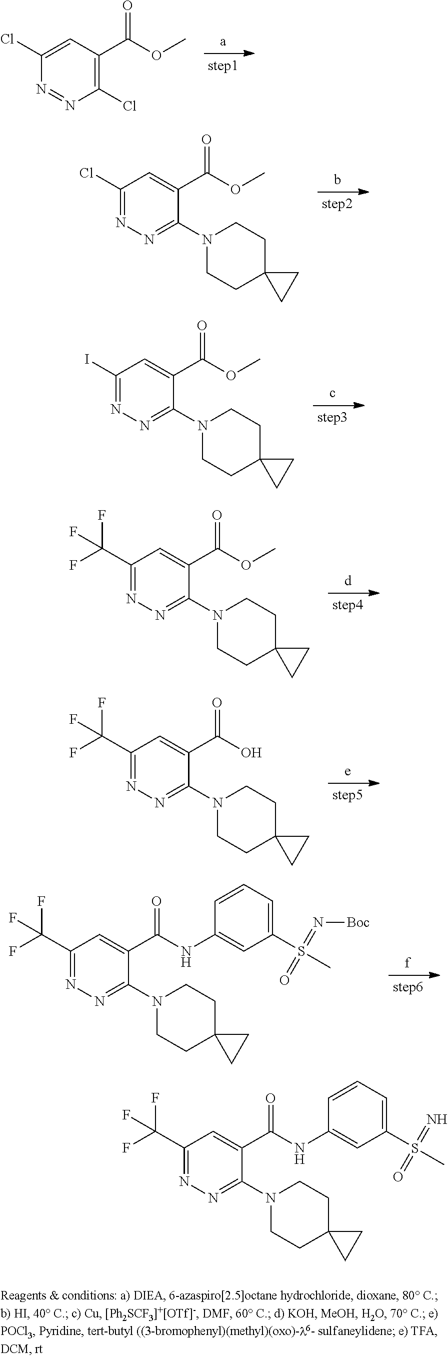

Step 1: methyl 6-chloro-3-(6-azaspiro[2.5]octan-6-yl)pyridazine-4-carboxylate (3): A mixture of methyl 3,6-dichloropyridazine-4-carboxylate (1.5 g, 7.2 mmol) and 6-azaspiro[2.5]octane hydrochloride (1.06 g, 7.2 mmol) in 1,4-dioxane (20 mL) was added DIEA (1.87 g, 14.4 mmol) at room temperature. The reaction mixture was heated at 100° C. for 16 hours. After the reaction was completed, the mixture was cooled to room temperature. The mixture was diluted with water (20 mL) and extracted with DCM (20 mL×3). The combine organic phases were washed with brine, dried over sodium sulfate, concentrated under vacuum. The residue was purified by flash column chromatography on silica gel (PE/EtOAc=3/1) to give methyl 6-chloro-3-(6-azaspiro[2.5]octan-6-yl)pyridazine-4-carboxylate (1.1 g, 54.37%) as a yellow oil. LCMS (ESI) calcd. for C₁₃H₁₇ClN₃O₂ [M+H]⁺ m/z 282.10, found 281.95.

Step 2: methyl 6-iodo-3-(6-azaspiro[2.5]octan-6-yl)pyridazine-4-carboxylate: To a solution of methyl 6-chloro-3-(6-azaspiro[2.5]octan-6-yl)pyridazine-4-carboxylate (1.1 g, 3.91 mmol) was added HI (55%, 10 mL) at room temperature. The mixture was heated at 40° C. for 10 hours. After the reaction was completed, the mixture was cooled to room temperature and extracted with DCM (20 mL×3). The combined organic phases were dried with Na₂SO₄ and concentrated under reduced pressure to give crude methyl 6-iodo-3-(6-azaspiro[2.5]octan-6-yl)pyridazine-4-carboxylate (800 mg, 54.9%) as a yellow solid which was used directly in next Step without further purification. LCMS (ESI) calcd. for C₁₃H₁₇IN₃O₂ [M+H]⁺ m/z 374.04, found 373.90.

Step 3: methyl 3-(6-azaspiro[2.5]octan-6-yl)-6-(trifluoromethyl)pyridazine-4-carboxylate: A mixture of methyl 6-iodo-3-(6-azaspiro[2.5]octan-6-yl)pyridazine-4-carboxylate (600 mg, 1.6 mmol) and Cu (30.7 mg, 4.8 mmol) and [Ph₂SCF₃]⁺[OTf]⁻ (123 mg, 3.2 mmol) in DMF (10 mL) was heated at 60° C. for 2 hours. After the reaction was completed, the mixture was cooled to room temperature, diluted with water (40 mL) and extracted with DCM (40 mL×3). The combined organic phases were dried with Na₂SO₄ and concentrated under reduced pressure. The residue was purified by flash column chromatography on silica gel (PE/EtOAc=1/1) to give methyl 3-(6-azaspiro[2.5]octan-6-yl)-6-(trifluoromethyl)pyridazine-4-carboxylate (330 mg, 65.6%) as a yellow solid. LCMS (ESI) calcd. for C₁₄H₁₇F₃N₃O₂ [M+H]⁺ m/z 316.13, found 316.00.

Step 4: 3-(6-azaspiro[2.5]octan-6-yl)-6-(trifluoromethyl)pyridazine-4-carboxylic acid: To a solution methyl 3-(6-azaspiro[2.5]octan-6-yl)-6-(trifluoromethyl)pyridazine-4-carboxylate (330 mg, 1.05 mmol) in MeOH/H₂O (10 mL) was added KOH (560 mg, 10 mmol) at room temperature. The mixture was heated at 70° C. for 6 hours. After the reaction was completed, the mixture was cooled to room temperature, diluted with water and extracted with DCM (20 mL). The aqueous phase was adjusted to pH=3 with 1N HCl and extracted with DCM (20 mL×3). The combined organic phases were dried with Na₂SO₄ and concentrated under reduced pressure to give 3-(6-azaspiro[2.5]octan-6-yl)-6-(trifluoromethyl)pyridazine-4-carboxylic acid (240 mg, 75.9%) as a white solid. LCMS (ESI) calcd. for C₁₃H₁₅F₃N₃O₂ [M+H]⁺ m/z 302.11, found 301.90.

Step 5: tert-butyl ((3-(3-(6-azaspiro[2.5]octan-6-yl)-6-(trifluoromethyl)pyridazine-4-carboxamido)phenyl)(methyl)(oxo)-16-sulfaneylidene)carbamate: A mixture of 3-(6-azaspiro[2.5]octan-6-yl)-6-(trifluoromethyl)pyridazine-4-carboxylic acid (240 mg, 0.8 mmol) and tert-butyl ((3-aminophenyl)(methyl)(oxo)-λ⁶-sulfaneylidene)carbamate (325 mg, 1.2 mmol) in pyridine (4 mL) was added POCl₃ (350 L) at room temperature. The mixture was stirred at room for 2 hours. Then the mixture was diluted with water (4 mL) and extracted with DCM (4 mL×3). The combine organic phases were washed with brine, dried over sodium sulfate, concentrated under vacuum. The residue was purified by flash column chromatography on silica gel (PE/EtOAc=1/1) to give tert-butyl ((3-(3-(6-azaspiro[2.5]octan-6-yl)-6-(trifluoromethyl)pyridazine-4-carboxamido)phenyl)(methyl)(oxo)--λ⁶-sulfaneylidene)carbamate (85 mg, 19.1%) as a yellow oil. LCMS (ESI) calcd. for C₂₅H₃₁F₃N₅O₄S [M+H]⁺ m/z 554.21, found 554.10.

Step 6: N-(3-(S-methylsulfonimidoyl)phenyl)-3-(6-azaspiro[2.5]octan-6-yl)-6-(trifluoromethyl)pyridazine-4-carboxamide: A solution of tert-butyl ((3-(3-(6-azaspiro[2.5]octan-6-yl)-6-(trifluoromethyl)pyridazine-4-carboxamido)phenyl)(methyl)(oxo)-16-sulfaneylidene)carbamate (85 mg, 0.153 mmol) in DCM (3 mL) was added TFA (1 mL) at room temperature. The reaction mixture was stirred at room temperature for 2 hours. After the reaction was completed, the solution was diluted with water (10 mL) and extracted with DCM (10 mL×3). The combine organic phases were washed with brine, dried over sodium sulfate and concentrated under vacuum. The residue was first purified by flash chromatography on silica gel (PE/EtOAc=1/1), then further purified by prep-HPLC (Gemini 5 um C₁₈ column, 150×21.2 mm, eluting with 30% to 90% MeCN/H₂O containing 0.1% formic acid) to obtain N-(3-(S-methylsulfonimidoyl)phenyl)-3-(6-azaspiro[2.5]octan-6-yl)-6-(trifluoromethyl)pyridazine-4-carboxamide (44 mg, purity 98.3%) as a white solid. ¹H NMR (400 MHz, DMSO-d₆) δ 11.08 (s, 1H), 8.29 (s, 1H), 8.02 (s, 1H), 7.91 (d, J=8.4 Hz, 1H), 7.70 (d, J=7.8 Hz, 1H), 7.62 (t, J=7.9 Hz, 1H), 4.25 (s, 1H), 3.71-3.67 (m, 4H), 3.06 (s, 3H), 1.46-1.42 (m, 4H), 0.35 (s, 4H). LCMS (ESI) calcd. for C₂₀H₂₃F₃N₅O₂S [M+H]⁺ m/z 454.15, found 454.10.

### Example 15

### 5-chloro-2-(4,4-difluoroazepan-1-yl)-6-methyl-N-(3-(S-methylsulfonimidoyl)phenyl)nicotinamide

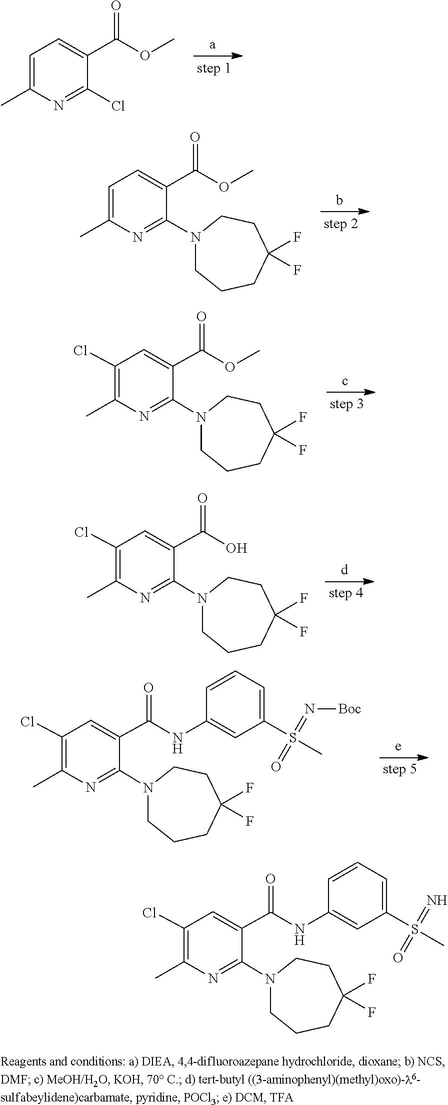

Step 1: methyl 2-(4,4-difluoroazepan-1-yl)-6-methylnicotinate: A mixture of methyl 2-chloro-6-methylnicotinate (1.0 g, 5.4 mmol), 4,4-difluoroazepane hydrochloride (1.11 g, 6.4 mmol) and DIEA (1.39 g, 10.8 mmol) in dioxane (20 mL) was heated at 80° C. for 5 hours. Then the mixture was concentrated, and the residue was purified by flash column chromatography on silica gel (PE/EtOAc=5/1) to give methyl 2-(4,4-difluoroazepan-1-yl)-6-methylnicotinate (720 mg, 42.6%). LCMS (ESI) calcd. for C₁₄H₁₈F₂N₂O₂ [M+H]⁺ m/z 285.14, found 285.15.

Step 2: methyl 5-chloro-2-(4,4-difluoroazepan-1-yl)-6-methylnicotinate: To a solution of methyl 2-(4,4-difluoroazepan-1-yl)-6-methylnicotinate (700 mg, 2.46 mmol) in DMF (10 mL) was added NCS (394 mg, 2.95 mmol). The mixture was stirred for 5 hours at room temperature. Then the mixture was diluted with water (40 mL) and extracted with DCM (30 mL×3). The combined organic layers were washed with water and brine, dried over Na₂SO₄, filtered, concentrated to give the crude. The crude was purified by silica gel column chromatography (PE/EtOAc, 4/1) to afford methyl 5-chloro-2-(4,4-difluoroazepan-1-yl)-6-methylnicotinate (680 mg, 78%) as a yellow solid. LCMS (ESI) calcd. for C₁₄H₁₈ClF₂N₂O₂ [M+H]⁺ m/z 319.10, found 319.00.

Step 3: 5-chloro-2-(4,4-difluoroazepan-1-yl)-6-methylnicotinic acid: To a solution of methyl 5-chloro-2-(4,4-difluoroazepan-1-yl)-6-methylnicotinate (650 mg, 2.03 mmol) in MeOH/H₂O (1/1, 10 mL) was added KOH (1.14 g, 20.4 mmol) at room temperature. The mixture was heated at 70° C. for 4 hours. After the reaction was completed, the mixture was cooled to room temperature and concentrated to remove MeOH. The aqueous phase was adjusted to pH=3-4 with 1N HCl and extracted with EtOAc (50 mL×3). The combined organic phases were washed with brine, dried with Na₂SO₄, and concentrated under reduced pressure to give 5-chloro-2-(4,4-difluoroazepan-1-yl)-6-methylnicotinic acid (600 mg, 86.9%). LCMS (ESI) calcd. for C₁₃H₁₆ClF₂N₂O₂ [M+H]⁺ m/z 305.09, found 304.90.

Step 4: tert-butyl ((3-(5-chloro-2-(4,4-difluoroazepan-1-yl)-6-methylnicotinamido)phenyl)(methyl)(oxo)-λ⁶-sulfaneylidene)carbamate: To a solution of 5-chloro-2-(4,4-difluoroazepan-1-yl)-6-methylnicotinic acid (120 mg, 0.39 mmol) and tert-butyl ((3-aminophenyl)(methyl)(oxo)-λ⁶-sulfaneylidene)carbamate (160 mg, 0.59 mmol) in pyridine (6 mL) was added POCl₃ (0.3 mL) dropwise at 0° C. The mixture was stirred at room temperature for 2 hours. LCMS showed the reaction was completed. The mixture was quenched with water (60 mL) and extracted with EtOAc (40 mL×3). The combine organic phases were washed with brine, dried over sodium sulfate, concentrated under vacuum. The residue was purified by flash column chromatography on silica gel (PE/EtOAc=1/2) to provide tert-butyl ((3-(5-chloro-2-(4,4-difluoroazepan-1-yl)-6-methylnicotinamido)phenyl)(methyl)(oxo)-_⁶-sulfaneylidene)carbamate (65 mg, 27%) as a yellow oil. LCMS (ESI) calcd. for C₂₅H₃₂ClF₂N₄O₄S [M+H]⁺ m/z 557.18, found 557.15.

Step 5: 5-chloro-2-(4,4-difluoroazepan-1-yl)-6-methyl-N-(3-(S-methylsulfonimidoyl)phenyl)nicotinamide: To a solution of tert-butyl ((3-(5-chloro-2-(4,4-difluoroazepan-1-yl)-6-methylnicotinamido) phenyl)(methyl)(oxo)-λ⁶-sulfaneylidene)carbamate (65 mg, 0.12 mmol) in DCM (3 mL) was added TFA (1 mL) at room temperature. The mixture was stirred at room temperature for 2 hours. LCMS showed the reaction was completed. The final mixture was concentrated in vacuum and purified by prep-HPLC (Gemini 5 um C₁₈ column, 150×21.2 mm, eluting with 50% to 80% MeCN/H₂O containing 0.1% formic acid) to afford 5-chloro-2-(4,4-difluoroazepan-1-yl)-6-methyl-N-(3-(S-methylsulfonimidoyl)phenyl)nicotinamide (10.1 mg, 18.8%) as a white solid. LCMS (ESI) calcd. for C₂₀H₂₄ClF₂N₄O₂S [M+H]⁺ m/z 457.13, found 457.00. ¹H NMR (400 MHz, DMSO-d₆) δ 0.74 (s, 1H), 8.32 (s, 1H), 7.94 (d, J=8.2 Hz, 1H), 7.77 (s, 1H), 7.66 (d, J=8.0 Hz, 1H), 7.58 (t, J=7.9 Hz, 1H), 3.64-3.57 (m, 2H), 3.42-3.38 (m, 2H), 3.09 (s, 3H), 2.45 (s, 3H), 2.35-2.25 (m, 2H), 2.02-1.89 (m, 2H), 1.87-1.80 (m, 2H).

### Example 16

### (R)-5-chloro-2-(4,4-difluoroazepan-1-yl)-6-methyl-N-(3-(S-methylsulfonimidoyl)phenyl)nicotinamide

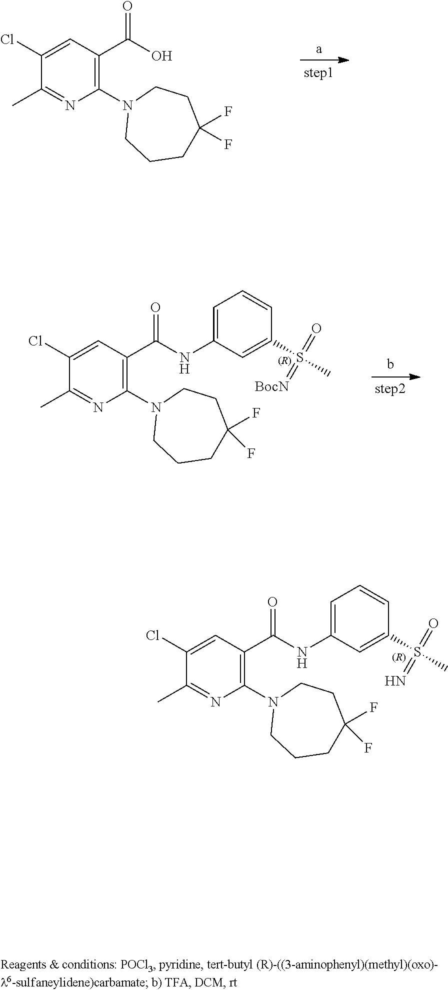

Step 1: tert-butyl (R)-((3-(5-chloro-2-(4,4-difluoroazepan-1-yl)-6-methylnicotinamido)phenyl)(methyl)(oxo)-λ⁶-sulfaneylidene)carbamate: A mixture of 5-chloro-2-(4,4-difluoroazepan-1-yl)-6-methylnicotinic acid (250 mg, 0.820 mmol) and tert-butyl (R)-((3-aminophenyl)(methyl)(oxo)-16-sulfaneylidene)carbamate (244 mg, 0.902 mmol) in pyridine (4 mL) was added POCl₃ (373 L) at 50° C. The reaction was monitored by LCMS. After the reaction was completed, the mixture was cooled to room temperature. The resulting solution was diluted with water (15 mL) and extracted with DCM (15 mL×3). The combined organic phases were washed with brine, dried over Na₂SO₄, concentrated under vacuum. The residue was purified by flash column chromatography on silica gel (PE/EtAOc=1/1) to give tert-butyl (R)-((3-(5-chloro-2-(4,4-difluoroazepan-1-yl)-6-methylnicotinamido)phenyl)(methyl)(oxo)-λ⁶-sulfaneylidene)carbamate (120 mg, 26.3%) as a yellow oil. LCMS (ESI) calcd. for C₂₅H₃₂ClF₂N₄O₄S [M+H]⁺ m/z 557.18, found 557.10.

Step 2: (R)-5-chloro-2-(4,4-difluoroazepan-1-yl)-6-methyl-N-(3-(S-methylsulfonimidoyl)phenyl)nicotinamide: A solution of tert-butyl (R)-((3-(5-chloro-2-(4,4-difluoroazepan-1-yl)-6-methylnicotinamido)phenyl)(methyl)(oxo)-λ⁶-sulfaneylidene)carbamate (120 mg, 0.210 mmol) in DCM (3 mL) was added TFA (1 mL) at room temperature. The reaction mixture was stirred at room temperature for 2 hours. After the reaction was completed, the filtrate was diluted with water (5 mL) and extracted with DCM (5 mL×3). The combined organic phases were washed with brine, dried over sodium sulfate, concentrated under vacuum. The residue was purified by prep-HPLC (Gemini 5 um C₁₈ column, 150×21.2 mm, eluting with 30% to 90% ACN-H₂O containing 0.1% formic acid) to obtain (R)-5-chloro-2-(4,4-difluoroazepan-1-yl)-6-methyl-N-(3-(S-methylsulfonimidoyl)phenyl)nicotinamide (67 mg, 69.8%, 100% ee) as a white solid. ¹H NMR (400 MHz, DMSO-d₆, ppm) δ 10.72 (s, 1H), 8.30 (s, 1H), 7.93 (d, J=8 Hz, 1H), 7.77 (s, 1H), 7.65 (d, J=8 Hz, 1H), 7.59-7.55 (m, 1H), 4.21 (s, 1H), 3.62-3.60 (m, 2H), 3.42-3.39 (m, 2H), 3.05 (s, 3H), 2.28 (s, 3H), 2.29-2.26 (m, 2H), 1.95-1.91 (m, 2H), 1.85-1.73 (m, 2H). LCMS (ESI) calcd. for C₂₀H₂₄ClF₂N₄O₂ [M+H]⁺ m/z 457.13, found 456.90.

### Example 17

### (S)-5-Chloro-2-(4,4-difluoroazepan-1-yl)-6-methyl-N-(3-(S-methylsulfonimidoyl)phenyl)nicotinamide

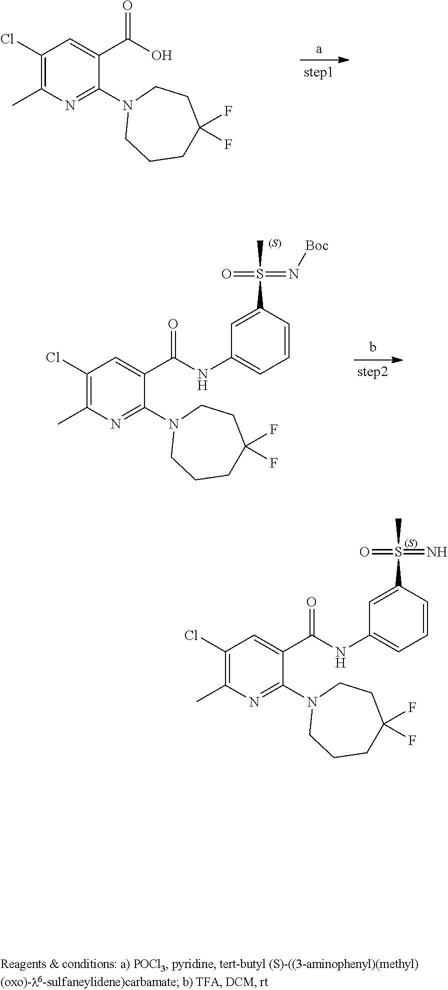

Step 1: tert-butyl (S)-((3-(5-chloro-2-(4,4-difluoroazepan-1-yl)-6-methylnicotinamido)phenyl)(methyl)(oxo)-λ⁶-sulfaneylidene)carbamate: A mixture of 5-chloro-2-(4,4-difluoroazepan-1-yl)-6-methylnicotinic acid (250 mg, 0.8224 mmol) and tert-butyl (S)-((3-aminophenyl)(methyl)(oxo)-λ⁶-sulfaneylidene)carbamate (266.447 mg, 0.9868 mmol) in pyridine (5 mL) was added dropwise added POCl₃ (500 L) dropwise at room temperature. The reaction was monitored by LCMS. After the reaction was completed, the resulting solution was diluted with water (20 mL) and extracted with DCM (20 mL×3). The combined organic phases were washed with brine, dried over sodium sulfate, concentrated under vacuum. The residue was purified by flash column chromatography on silica gel (PE/EtOAc=2/1) to give tert-butyl (S)-((3-(5-chloro-2-(4,4-difluoroazepan-1-yl)-6-methylnicotinamido)phenyl)(methyl)(oxo)-λ⁶-sulfaneylidene)carbamate (150 mg, 32.80%) as a white solid. LCMS (ESI) calcd. for C₂₅H₃₂ClF₂N₄O₄S [M+H]⁺ m/z 557.17, found 557.05.

Step 2: (S)-5-chloro-2-(4,4-difluoroazepan-1-yl)-6-methyl-N-(3-(S-methylsulfonimidoyl)phenyl)nicotinamide: A solution of tert-butyl (S)-((3-(5-chloro-2-(4,4-difluoroazepan-1-yl)-6-methylnicotinamido)phenyl)(methyl)(oxo)-λ⁶-sulfaneylidene)carbamate (150 mg, 0.27 mmol) in DCM (3 mL) was added TFA (1 mL) at room temperature. The reaction mixture was stirred at room temperature for 2 hours. After the reaction was completed, the mixture was diluted with water (10 mL) and extracted with DCM (10 mL×3). The combined organic phases were washed with brine, dried over sodium sulfate, concentrated under vacuum. The residue was purified by prep-HPLC (Gemini 5 um C₁₈ column, 150×21.2 mm, eluting with 30% to 90% ACN-H₂O containing 0.1% formic acid) to give (S)-5-chloro-2-(4,4-difluoroazepan-1-yl)-6-methyl-N-(3-(S-methylsulfonimidoyl)phenyl)nicotinamide (54.5 mg, 44.3%) as a white solid. ¹H NMR (400 MHz, DMSO-d₆) δ 10.74 (s, 1H), 8.31 (s, 1H), 7.94 (d, J=8.4 Hz, 1H), 7.77 (s, 1H), 7.66 (d, J=8.0 Hz, 1H), 7.58 (t, J=7.9 Hz, 1H), 3.61 (dd, J=6.4, 3.6 Hz, 2H), 3.40 (s, 2H), 3.09 (s, 3H), 2.45 (s, 3H), 2.30 (d, J=14.7 Hz, 2H), 1.95 (t, J=12.2 Hz, 2H), 1.88-1.78 (m, 2H). LCMS (ESI) calcd. for C₂₀H₂₄ClF₂N₄O₂S [M+H]⁺ m/z 457.12, found 457.20.

### Example 18

### (R)-3-(4,4-difluoroazepan-1-yl)-N-(3-(S-methylsulfonimidoyl)phenyl)-6-(trifluoromethyl)pyridazine-4-carboxamide

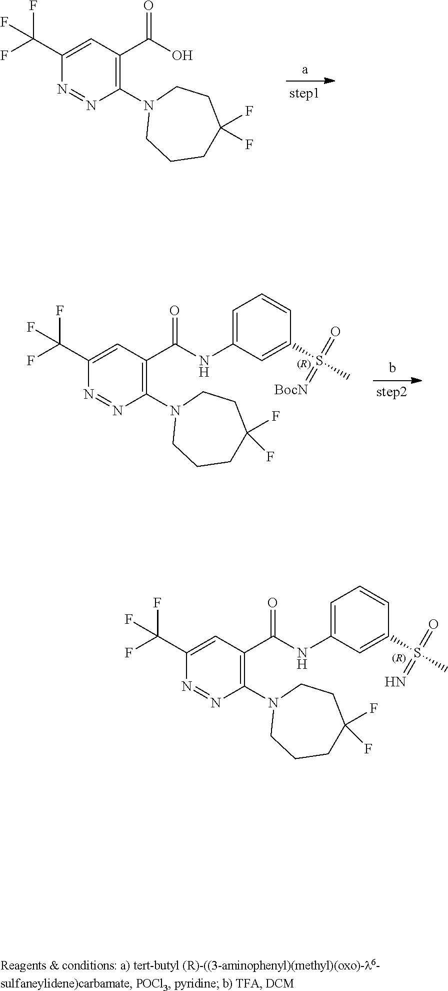

Step 1: tert-butyl (R)-((3-(3-(4,4-difluoroazepan-1-yl)-6-(trifluoromethyl)pyridazine-4-carboxamido)phenyl)(methyl)(oxo)-λ⁶-sulfaneylidene)carbamate: To a solution of 3-(4,4-difluoroazepan-1-yl)-6-(trifluoromethyl)pyridazine-4-carboxylic acid (100 mg, 0.31 mmol) and tert-butyl (R)-((3-aminophenyl)(methyl)(oxo)-λ⁶-sulfaneylidene)carbamate (91 mg, 0.34 mmol) in pyridine (5 mL) was added POCl₃ (50 L) dropwise at 0° C. The mixture was stirred at room temperature for 2 hours. LCMS showed the reaction was completed. The mixture was quenched with water (30 mL) and extracted with EtOAc (30 mL×3). The combine organic phases were washed with brine, dried over sodium sulfate, concentrated under vacuum. The residue was purified by flash column chromatography on silica gel (PE/EtOAc=3/1) to provide tert-butyl (R)-((3-(3-(4,4-difluoroazepan-1-yl)-6-(trifluoromethyl)pyridazine-4-carboxamido)phenyl)(methyl)(oxo)-λ⁶-sulfaneylidene)carbamate (82 mg, 45.8%) as a yellow solid. LCMS (ESI) calcd. for C₂₄H₂₉F₅N₅O₄S [M+H]⁺ m/z 578.19, found 578.05.

Step 2: (R)-3-(4,4-difluoroazepan-1-yl)-N-(3-(S-methylsulfonimidoyl)phenyl)-6-(trifluoromethyl)pyridazine-4-carboxamide: To a solution of tert-butyl (R)-((3-(3-(4,4-difluoroazepan-1-yl)-6-(trifluoromethyl)pyridazine-4-carboxamido)phenyl)(methyl)(oxo)-λ⁶-sulfaneylidene)carbamate (90 mg, 0.16 mmol) in DCM (1.5 mL) was added TFA (0.5 mL) at room temperature. The mixture was stirred at room temperature for 1 hours. LCMS showed the reaction was completed. The final mixture was concentrated in vacuum and purified by prep-HPLC (Gemini 5 um C₁₈ column, 150×21.2 mm, eluting with 30% to 90% MeCN/H₂O containing 0.1% formic acid) to afford (R)-3-(4,4-difluoroazepan-1-yl)-N-(3-(S-methylsulfonimidoyl)phenyl)-6-(trifluoromethyl)pyridazine-4-carboxamide (38 mg, 50%, 99% ee) as a white solid. LCMS (ESI) calcd. for C₁₉H₂₁F₅N₅O₂S [M+H]⁺ m/z 478.14, found 478.25. ¹H NMR (400 MHz, DMSO-d₆) δ 11.07 (s, 1H), 8.26 (s, 1H), 8.08 (s, 1H), 7.94 (d, J=8.0 Hz, 1H), 7.71 (d, J=7.8 Hz, 1H), 7.62 (t, J=7.9 Hz, 1H), 4.25 (s, 1H), 3.87 (s, 2H), 3.58 (t, J=5.7 Hz, 2H), 3.06 (s, 3H), 2.39 (s, 2H), 2.19-1.98 (m, 2H), 1.96-1.85 (d, J=5.1 Hz, 2H).

### Example 19

### (S)-3-(4,4-difluoroazepan-1-yl)-N-(3-(S-methylsulfonimidoyl)phenyl)-6-(trifluoromethyl)pyridazine-4-carboxamide

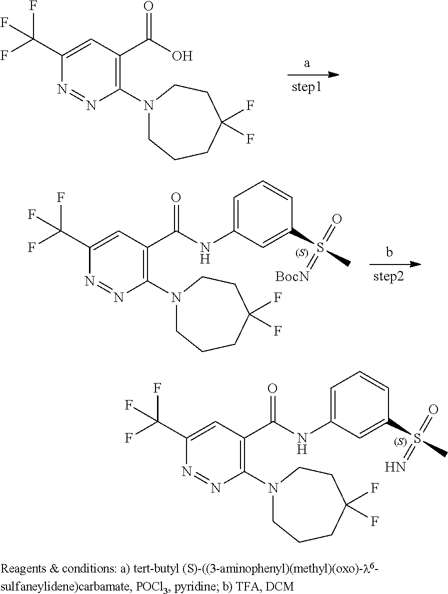

Step 1: tert-butyl (S)-((3-(3-(4,4-difluoroazepan-1-yl)-6-(trifluoromethyl)pyridazine-4-carboxamido)phenyl)(methyl)(oxo)-λ⁶-sulfaneylidene)carbamate To a solution of 3-(4,4-difluoroazepan-1-yl)-6-(trifluoromethyl)pyridazine-4-carboxylic acid (100 mg, 0.31 mmol) and tert-butyl (S)-((3-aminophenyl)(methyl)(oxo)-λ⁶-sulfaneylidene)carbamate (91 mg, 0.34 mmol) in pyridine (5 mL) was added POCl₃ (50 L) dropwise at 0° C. The mixture was stirred at room temperature for 2 hours. LCMS showed the reaction was completed. The mixture was quenched with water (20 mL) and extracted with EtOAc (20 mL×3). The combine organic phases were washed with brine, dried over sodium sulfate, concentrated under vacuum. The residue was purified by flash column chromatography on silica gel (PE/EtOAc=1/2) to provide tert-butyl (S)-((3-(3-(4,4-difluoroazepan-1-yl)-6-(trifluoromethyl)pyridazine-4-carboxamido)phenyl)(methyl)(oxo)-λ⁶-sulfaneylidene)carbamate (90 mg, 50.8%) as a yellow solid. LCMS (ESI) calcd. for C₁₉H₂₁F₅N₅O₂S [M-Boc+H]⁺ m/z 478.14, found 478.05.

Step 2: (S)-3-(4,4-difluoroazepan-1-yl)-N-(3-(S-methylsulfonimidoyl)phenyl)-6-(trifluoromethyl)pyridazine-4-carboxamide: To a solution of tert-butyl (S)-((3-(3-(4,4-difluoroazepan-1-yl)-6-(trifluoromethyl)pyridazine-4-carboxamido)phenyl)(methyl)(oxo)-λ⁶-sulfaneylidene)carbamate (90 mg, 0.16 mmol) in DCM (1.5 mL) was added TFA (0.5 mL) at room temperature. The mixture was stirred at room temperature for 1 hour. LCMS showed the reaction was completed. The final mixture was concentrated in vacuum and purified by prep-HPLC (Gemini 5 um C₁₈ column, 150×21.2 mm, eluting with 30% to 90% MeCN/H₂O containing 0.1% formic acid) to afford (S)-3-(4,4-difluoroazepan-1-yl)-N-(3-(S-methylsulfonimidoyl)phenyl)-6-(trifluoromethyl)pyridazine-4-carboxamide (53 mg, 71.6%) as a white solid. ¹H NMR (400 MHz, DMSO-d₆) δ 11.08 (s, 1H), 8.28 (s, 1H), 8.08 (s, 1H), 7.95 (d, J=7.9 Hz, 1H), 7.72 (d, J=7.8 Hz, 1H), 7.63 (t, J=7.9 Hz, 1H), 3.87 (s, 2H), 3.58 (t, J=5.4 Hz, 2H), 3.10 (s, 3H), 2.45-2.31 (m, 2H), 2.12-2.00 (m, 2H), 1.98-1.88 (m, 2H). LCMS (ESI) calcd. for C₁₉H₂₁F₅N₅O₂S [M+H]⁺ m/z 478.14, found 478.50.

### Example 20

### 3-(4,4-difluoroazepan-1-yl)-5-methyl-N-(3-(S-methylsulfonimidoyl)phenyl)-6-phenylpyridazine-4-carboxamide

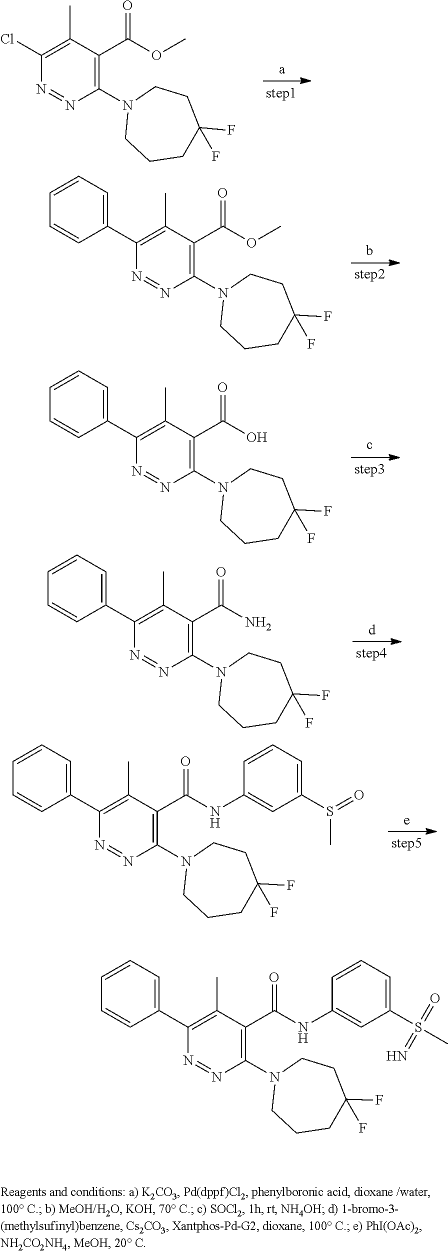

Step 1: methyl 3-(4,4-difluoroazepan-1-yl)-5-methyl-6-phenylpyridazine-4-carboxylate: A mixture of methyl 6-chloro-3-(4,4-difluoroazepan-1-yl)-5-methylpyridazine-4-carboxylate (600 mg, 1.88 mmol) and phenylboronic acid (1.146 g, 9.401 mmol) in 1,4-dioxane/H₂O (4/1, 10 mL), potassium carbonate (779 mg, 5.641 mmol) and [1,1′-bis(diphenylphosphino)ferrocene]dichloropalladium (II) (137 mg, 0.188 mmol) was heated at 100° C. for 6 h under an atmosphere of N₂. The reaction was monitored by LCMS. After the reaction was completed, the mixture was cooled to room temperature. The resulting solution was diluted with water (20 mL) and extracted with DCM (20 mL×3). The combined organic phases were washed with brine, dried over Na₂SO₄, concentrated under vacuum. The residue was purified by flash column chromatography on silica gel (PE/EtAOc=3/1) to give methyl 3-(4,4-difluoroazepan-1-yl)-5-methyl-6-phenylpyridazine-4-carboxylate (600 mg, 88.2%) as a white oil. LCMS (ESI) calcd. for C₁₉H₂₂F₂N₃O₂ [M+H]⁺ m/z 362.17, found 362.00.

Step 2: 3-(4,4-difluoroazepan-1-yl)-5-methyl-6-phenylpyridazine-4-carboxylic acid: To a solution methyl 3-(4,4-difluoroazepan-1-yl)-5-methyl-6-phenylpyridazine-4-carboxylate (0.550 g, 1.519 mmol) in MeOH/H₂O (1/1, 10 mL) was added KOH (0.852 g, 15.19 mmol) at room temperature. The mixture was heated at 70° C. for 6 hours. After the reaction was completed, the mixture was cooled to room temperature and extracted with DCM (10 mL). Then the aqueous phase was adjusted to pH=3 with 1N HCl and extracted with DCM (10 mL×3). The combined organic phases were dried with Na₂SO₄ and concentrated under reduced pressure to give 3-(4,4-difluoroazepan-1-yl)-5-methyl-6-phenylpyridazine-4-carboxylic acid (500 mg, 65.8%) as a white solid. LCMS (ESI) calcd. for C₁₈H₂₀F₂N₃O₂ [M+H]⁺ m/z 348.15, found 348.00.

Step 3: 3-(4,4-difluoroazepan-1-yl)-5-methyl-6-phenylpyridazine-4-carboxamide: A solution of 3-(4,4-difluoroazepan-1-yl)-5-methyl-6-phenylpyridazine-4-carboxylic acid (450 mg, 1.293 mmol) in SOCl₂ (5 mL) was stirred at room temperature for 1 hour. After the reaction was completed, the reaction mixture was concentrated. The residue was diluted with THE and added dropwise to a stirred solution of ammonium hydroxide (5 mL). Then the mixture was stirred at room temperature for 1 hour. After the reaction was completed, the mixture was diluted with water (10 mL) and extracted with DCM (10 mL×3). The combined organic phases were washed with brine, dried over sodium sulfate, concentrated under vacuum. The residue was purified by flash column chromatography on silica gel (PE/EtOAc=1/1) to give 3-(4,4-difluoroazepan-1-yl)-5-methyl-6-phenylpyridazine-4-carboxamide (420 mg, 93.8%) as a white solid. LCMS (ESI) calcd. for C₁₈H₂₁F₂N₄O [M+H]⁺ m/z 347.17, found 347.05.

Step 4. 3-(4,4-difluoroazepan-1-yl)-5-methyl-N-(3-(methylsulfinyl)phenyl)-6-phenylpyridazine-4-carboxamide: A solution of 3-(4,4-difluoroazepan-1-yl)-5-methyl-6-phenylpyridazine-4-carboxamide (400 mg, 1.152 mmol) in dioxane (8 mL) was added 1-bromo-3-(methylsulfinyl)benzene (946 mg, 3.456 mmol), cesium carbonate (975 mg, 2.995 mmol) and Xantphos-Pd-G2 (204 mg, 0.230 mmol) at room temperature. The reaction mixture was stirred at 100° C. for 16 hours under nitrogen. The reaction was monitored by LCMS. After the reaction was completed, the filtrate was diluted with water (20 mL) and extracted with DCM (20 mL×3). The combined organic phases were washed with brine, dried over sodium sulfate, concentrated under vacuum. The residue was purified by flash column chromatography on silica gel (PE/EtOAc=1/1) to give 3-(4,4-difluoroazepan-1-yl)-5-methyl-N-(3-(methylsulfinyl)phenyl)-6-phenylpyridazine-4-carboxamide (120 mg, 21.5%) as a white solid. LCMS (ESI) calcd. for C₂₅H₂₇F₂N₄O₂S [M+H]⁺ m/z 485.18, found 485.10.

Step 5: 3-(4,4-difluoroazepan-1-yl)-5-methyl-N-(3-(S-methylsulfonimidoyl)phenyl)-6-phenylpyridazine-4-carboxamide: To a solution of 3-(4,4-difluoroazepan-1-yl)-5-methyl-N-(3-(methylsulfinyl)phenyl)-6-phenylpyridazine-4-carboxamide (120 mg, 0.247 mmol) in MeOH (5 mL) was added PhI(OAc)₂ (199 mg, 0.618 mmol) and ammonium carbamate (58 mg, 0.741 mmol) at room temperature. The reaction mixture was heated at 70° C. for 1 hour. After the reaction was completed, the mixture was cooled to room temperature, diluted with water (5 mL), and extracted with DCM (5 mL×3). The combined organic phases were washed with brine, dried over sodium sulfate, concentrated under vacuum. The residue was first purified by flash column chromatography on silica gel (PE/EtOAc=1/1), then further purified by prep-HPLC (Gemini 5 um C₁₈ column, 150×21.2 mm, eluting with 30% to 90% ACN-H₂O containing 0.1% formic acid) to afford 3-(4,4-difluoroazepan-1-yl)-5-methyl-N-(3-(S-methylsulfonimidoyl)phenyl)-6-phenylpyridazine-4-carboxamide (28 mg, 20%) as a white solid. ¹H NMR (400 MHz, DMSO-d₆, ppm) δ 11.08 (s, 1H), 8.32 (s, 1H), 7.89 (d, J=8.4 Hz, 1H), 7.70 (d, J=8 Hz, 1H), 7.62 (d, J=8 Hz, 1H), 7.59-7.57 (m, 2H), 7.55-7.51 (m, 2H), 7.49-7.46 (m, 1H), 4.24 (s, 1H), 3.78-3.76 (m, 2H), 3.67-3.64 (m, 2H), 3.06 (s, 3H), 2.35-2.32 (m, 2H), 2.19 (s, 3H), 2.06-2.04 (m, 2H), 1.89-1.87 (m, 2H). LCMS (ESI) calcd. for C₂₅H₂₈F₂N₅O₂S [M+H]⁺ m/z 500.20, found 499.95.

### Example 21

### 4-(4,4-difluoroazepan-1-yl)-N-(3-(S-methylsulfonimidoyl)phenyl)-6-(trifluoromethyl)pyridazine-3-carboxamide

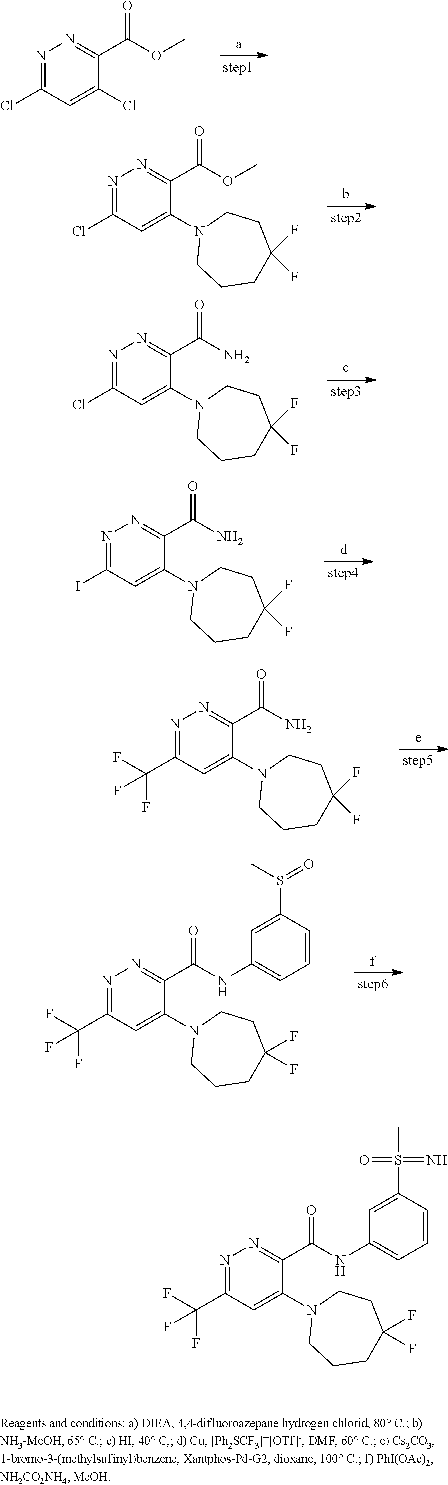

Step 1: methyl 6-chloro-4-(4,4-difluoroazepan-1-yl)pyridazine-3-carboxylate: To a solution of methyl 4,6-dichloropyridazine-3-carboxylate (5.0 g, 24.3 mmol) and 4,4-difluoroazepane hydrogen chloride (4.98 g, 29.2 mmol) in 1,4-dioxane (50 mL) was added DIEA (6.27 g, 48.6 mmol). The mixture was heated at 80° C. for 4 hours. LCMS showed the reaction was completed. The mixture was concentrated and purified by flash column chromatography on silica gel (PE/EtOAc=15/1) to give the methyl 6-chloro-4-(4,4-difluoroazepan-1-yl)pyridazine-3-carboxylate (4.8 g, 64.9%) as a white solid. LCMS (ESI) calcd. for C₁₂H₁₅ClF₂N₃O₂ [M+H]⁺ m/z 306.08, found 305.90.

Step 2: 6-chloro-4-(4,4-difluoroazepan-1-yl)pyridazine-3-carboxamide: A solution of methyl 6-chloro-4-(4,4-difluoroazepan-1-yl)pyridazine-3-carboxylate (4.8 g, 15.7 mmol) in NH₃-MeOH (7M, 15 mL) was heated at 60° C. for 16 hours in a high-pressure reactor. The reaction mixture was concentrated to give crude 6-chloro-4-(4,4-difluoroazepan-1-yl)pyridazine-3-carboxamide (4.8 g) which was used in the next Step without purification. LCMS (ESI) calcd. for C₁₁H₁₄ClF₂N₄O [M+H]⁺ m/z 291.08, found 290.95.

Step 3: 4-(4,4-difluoroazepan-1-yl)-6-iodopyridazine-3-carboxamide: A solution of 6-chloro-4-(4,4-difluoroazepan-1-yl)pyridazine-3-carboxamide (2.5 g, 8.62 mmol) in HI (10 mL, 57% aqueous solution) was heated at 40° C. for 5 hours. After cooling to ambient temperature, the precipitate was collected by filtration, washed with water, and dried under vacuum to give the 4-(4,4-difluoroazepan-1-yl)-6-iodopyridazine-3-carboxamide (2.2 g, 66.8%) as a white solid. LCMS (ESI) calcd. for C₁₁H₁₄F₂IN₄O [M+H]⁺ m/z 383.02, found 382.85.

Step 4: 4-(4,4-difluoroazepan-1-yl)-6-(trifluoromethyl)pyridazine-3-carboxamide: A mixture of 4-(4,4-difluoroazepan-1-yl)-6-iodopyridazine-3-carboxamide (2.2 g, 5.76 mmol), Cu (1.1 g, 17.28 mmol) and [Ph₂SCF₃]⁺[OTf]⁻ (4.6 g, 11.52 mmol) in DMF (15 mL) was heated at 60° C. for 5 hours. LCMS showed the reaction was completed. Then the mixture was filtered through celite. The filtrate was diluted with water (100 mL) and extracted with EtOAc (80 mL×3). The combine organic phases were washed with brine, dried over sodium sulfate, concentrated, and purified by silica gel column chromatography (eluting with EtOAc/PE=5/1) to give 4-(4,4-difluoroazepan-1-yl)-6-(trifluoromethyl)pyridazine-3-carboxamide (1.3 g, 69.5%) as a yellow oil. LCMS (ESI) calcd. for C₁₂H₁₄F₅N₄O [M+H]⁺ m/z 325.11, found 324.95.

Step 5: 4-(4,4-difluoroazepan-1-yl)-N-(3-(methylsulfinyl)phenyl)-6-(trifluoromethyl)pyridazine-3-carboxamide: A solution of 4-(4,4-difluoroazepan-1-yl)-6-(trifluoromethyl)pyridazine-3-carboxamide (200 mg, 0.60 mmol), 1-bromo-3-(methylsulfinyl)benzene (262 mg, 1.2 mmol), Cs₂CO₃ (587 mg, 1.8 mmol) and Xantphos-Pd-G2 (55 mg, 0.06 mmol) in 1,4-dioxane (6 mL) was heated at 100° C. for overnight under an atmosphere of N₂. LCMS showed the reaction was completed. The mixture was filtered through celite and the filtrate was concentrated under vacuum. The residue was purified by flash column chromatography on silica gel (PE/EtOAc=4/1) to give 4-(4,4-difluoroazepan-1-yl)-N-(3-(methylsulfinyl)phenyl)-6-(trifluoromethyl)pyridazine-3-carboxamide (120 mg, 43%) as an white oil. LCMS (ESI) calcd. for C₁₉H₂₀F₅N₄O₂S [M+H]⁺ m/z 463.12, found 463.00.

Step 6: 4-(4,4-difluoroazepan-1-yl)-N-(3-(S-methylsulfonimidoyl)phenyl)-6-(trifluoromethyl)pyridazine-3-carboxamide: To a solution of 4-(4,4-difluoroazepan-1-yl)-N-(3-(methylsulfinyl)phenyl)-6-(trifluoromethyl)pyridazine-3-carboxamide (120 mg, 0.25 mmol) in MeOH (5 mL) was added PhI(OAc)₂ (347.49 mg, 1.08 mmol) and ammonium carbamate (100.98 mg, 1.29 mmol) at room temperature. The reaction mixture was heated at 70° C. for 3 hours. Then the mixture was cooled to room temperature, diluted with water (20 mL) and extracted with DCM (30 mL×3). The combined organic phases were washed with brine, dried over sodium sulfate, concentrated under vacuum. The residue was by prep-HPLC (Gemini 5 um C₁₈ column, 150×21.2 mm, eluting with 50% to 55% ACN-H₂O containing 0.1% FA) to afford 4-(4,4-difluoroazepan-1-yl)-N-(3-(S-methylsulfonimidoyl)phenyl)-6-(trifluoromethyl)pyridazine-3-carboxamide (15 mg, 12.1%) as a white solid. ¹H NMR (400 MHz, DMSO-d₆) δ 11.34 (s, 1H), 8.38 (s, 1H), 7.99 (d, J=8.1 Hz, 1H), 7.71 (d, J=7.8 Hz, 1H), 7.63 (t, J=7.9 Hz, 1H), 7.43 (s, 1H), 4.25 (s, 1H), 3.67 (s, 2H), 3.56 (t, J=5.7 Hz, 2H), 3.07 (s, 3H), 2.37-2.23 (m, 2H), 2.12-1.00 (m, 2H), 1.94-1.84 (s, 2H). LCMS (ESI) calcd. for C₁₉H₂₁F₅N₅O₂S [M+H]⁺ m/z 478.14, found 478.05.

### Example 22

### 6-cyclopropyl-3-(4,4-difluoroazepan-1-yl)-5-methyl-N-(3-(S-methylsulfonimidoyl)phenyl)pyridazine-4-carboxamide

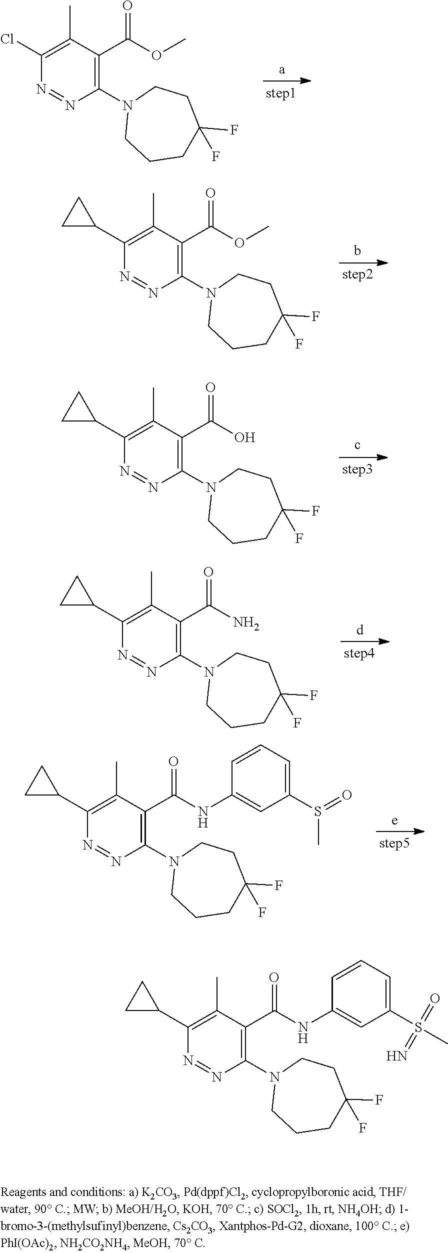

Step 1. methyl 6-cyclopropyl-3-(4,4-difluoroazepan-1-yl)-5-methylpyridazine-4-carboxylate: A mixture of methyl 6-chloro-3-(4,4-difluoroazepan-1-yl)-5-methylpyridazine-4-carboxylate (600 mg, 1.880 mmol) and cyclopropylboronic acid (323 mg, 3.760 mmol) in THE (10 mL) was added potassium phosphate (797 mg, 3.760 mmol) and [1,1′-bis(diphenylphosphino)ferrocene]dichloropalladium(II) (137 mg, 0.188 mmol) under nitrogen at room temperature. After addition, the reaction mixture was heated at 90° C. in a microwave reactor for 1 hour. After the reaction was completed, the mixture was cooled to room temperature. The resulting solution was diluted with water (20 mL) and extracted with DCM (20 mL×3). The combined organic phases were washed with brine, dried over Na₂SO₄, concentrated under vacuum. The residue was purified by flash column chromatography on silica gel (PE/EtAOc=3/1) to give methyl 6-cyclopropyl-3-(4,4-difluoroazepan-1-yl)-5-methylpyridazine-4-carboxylate (550 mg, 90.0%) as a white oil. LCMS (ESI) calcd. for C₁₆H₂₂F₂N₃O₂ [M+H]⁺ m/z 326.17, found 326.00.

Step 2: 6-cyclopropyl-3-(4,4-difluoroazepan-1-yl)-5-methylpyridazine-4-carboxylic acid: To a solution to methyl 6-cyclopropyl-3-(4,4-difluoroazepan-1-yl)-5-methylpyridazine-4-carboxylate (500 mg, 1.538 mmol) in MeOH/H₂O (1/1, 10 mL) was added KOH (861 mg, 15.38 mmol) at room temperature. The mixture was heated at 70° C. for 6 hours. After the reaction was completed, the mixture was cooled to room temperature and extracted with DCM (10 mL). Then the aqueous phase was adjusted to pH=3 with 1N HCl and extracted with DCM (10 mL×3). The combined organic phases were dried with Na₂SO₄ and concentrated under reduced pressure to give 6-cyclopropyl-3-(4,4-difluoroazepan-1-yl)-5-methylpyridazine-4-carboxylic acid (380 mg, 79.5%) as a white solid. LCMS (ESI) calcd. for C₁₅H₂₀F₂N₃O₂ [M+H]⁺ m/z 312.15, found 312.00.

Step 3: 6-cyclopropyl-3-(4,4-difluoroazepan-1-yl)-5-methylpyridazine-4-carboxamide: A solution of 6-cyclopropyl-3-(4,4-difluoroazepan-1-yl)-5-methylpyridazine-4-carboxylic acid (350 mg, 1.125 mmol) in SOCl₂ (5 mL) was stirred at room temperature for 1 hour. After the reaction was completed, the reaction mixture was concentrated under vacuum. The residue was dissolved in THE (3 mL) and added dropwise to a stirred solution of ammonium hydroxide (5 mL). Then the mixture was stirred at room temperature for 1 hour. After the reaction was completed, the filtrate was diluted with water (10 mL) and extracted with DCM (10 mL×3). The combined organic phases were washed with brine, dried over sodium sulfate, concentrated under vacuum. The residue was purified by flash column chromatography on silica gel (PE/EtOAc=1/1) to give 6-cyclopropyl-3-(4,4-difluoroazepan-1-yl)-5-methylpyridazine-4-carboxamide (280 mg, 80.4%) as a white solid. LCMS (ESI) calcd. for C₁₅H₂₁F₂N₄O [M+H]⁺ m/z 311.17, found 311.05.

Step 4: 6-cyclopropyl-3-(4,4-difluoroazepan-1-yl)-5-methyl-N-(3-(methylsulfinyl)phenyl)pyridazine-4-carboxamide: A solution of give 6-cyclopropyl-3-(4,4-difluoroazepan-1-yl)-5-methylpyridazine-4-carboxamide (250 mg, 0.803 mmol) in dioxane (8 mL) was added 1-bromo-3-(methylsulfinyl)benzene (526 mg, 2.411 mmol), cesium carbonate (678 mg, 2.087 mmol) and Xantphos-Pd-G2 (143 mg, 0.161 mmol) at room temperature. The reaction mixture was heated at 100° C. under nitrogen for 16 hours. After the reaction was completed, the mixture was diluted with water (20 mL) and extracted with DCM (20 mL×3). The combined organic phases were washed with brine, dried over sodium sulfate, concentrated under vacuum. The residue was purified by flash column chromatography on silica gel (PE/EtOAc=1/1) to give 6-cyclopropyl-3-(4,4-difluoroazepan-1-yl)-5-methyl-N-(3-(methylsulfinyl)phenyl)pyridazine-4-carboxamide (90 mg, 25.1%) as a white solid. LCMS (ESI) calcd. for C₂₂H₂₇F₂N₄O₂S [M+H]⁺ m/z 449.18, found 450.10.

Step 5: 6-cyclopropyl-3-(4,4-difluoroazepan-1-yl)-5-methyl-N-(3-(S-methylsulfonimidoyl)phenyl)pyridazine-4-carboxamide: To a solution of 6-cyclopropyl-3-(4,4-difluoroazepan-1-yl)-5-methyl-N-(3-(methylsulfinyl)phenyl)pyridazine-4-carboxamide (90 mg, 0.200 mmol) in MeOH (5 mL) was added PhI(OAc)₂ (161 mg, 0.501 mmol) and ammonium carbamate (46 mg, 0.600 mmol) at room temperature. The reaction mixture was heated at 70° C. for 1 hour. After the reaction was completed, the mixture was cooled to room temperature, diluted with water (15 mL), and extracted with DCM (15 mL×3). The combined organic phases were washed with brine, dried over sodium sulfate, concentrated under vacuum. The residue was first purified by flash column chromatography on silica gel (PE/EtOAc=1/1), then further purified by prep-HPLC (Gemini 5 um C₁₈ column, 150×21.2 mm, eluting with 30% to 90% ACN-H₂O containing 0.1% formic acid) to afford 6-cyclopropyl-3-(4,4-difluoroazepan-1-yl)-5-methyl-N-(3-(S-methylsulfonimidoyl)phenyl)pyridazine-4-carboxamide (52 mg, 56%) as a white solid. ¹H NMR (400 MHz, DMSO-d₆) δ 11.11 (s, 1H), 8.39 (s, 1H), 7.92 (d, J=8 Hz, 1H), 7.77 (d, J=7.2 Hz, 1H), 7.70-7.66 (m, 1H), 3.67-3.63 (m, 2H), 3.58-3.55 (m, 2H), 2.50 (s, 3H), 2.33 (s, 3H), 2.29-2.15 (m, 3H), 2.01-2.00 (m, 2H), 1.83-1.81 (m, 2H), 1.01-0.98 (m, 4H). LCMS (ESI) calcd. for C₂₂H₂₈F₂N₅O₂S [M+H]⁺ m/z 464.20, found 464.00.

### Example 23

### (S)-6-(Cyclopropylmethyl)-2-(4,4-difluoroazepan-1-yl)-N-(3-(S-methylsulfonimidoyl)phenyl)nicotinamide

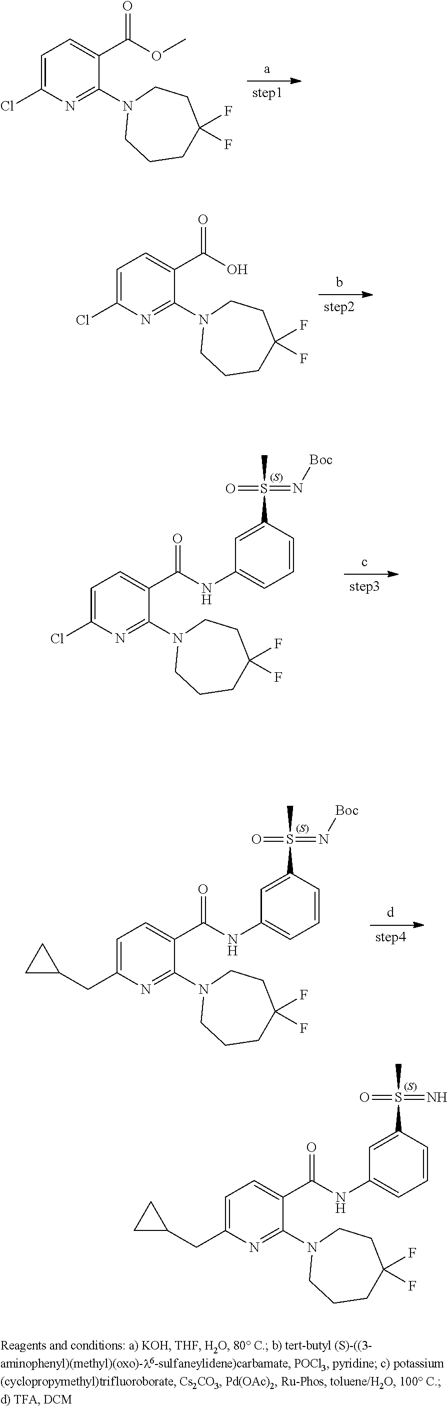

Step 1: 6-chloro-2-(4,4-difluoroazepan-1-yl)nicotinic acid: To a solution of methyl 6-chloro-2-(4,4-difluoroazepan-1-yl)nicotinate (200 mg, 0.66 mmol) in THF/H₂O (1/1, 10 mL) was added KOH (368 mg, 6.6 mmol) at room temperature. The mixture was heated at 80° C. for 16 hours. After the reaction was complete, the mixture was concentrated to remove most of the THF. The aqueous phase was adjusted to pH=3-4 with 1N HCl then extracted with EtOAc (20 mL×3). The combined organic phases were washed with brine, dried with Na₂SO₄, concentrated under reduced pressure to afford 6-chloro-2-(4,4-difluoroazepan-1-yl)nicotinic acid (180 mg, 94.7% yield) as a white solid. LCMS (ESI) calcd. for C₁₂H₁₄ClF₂N₂O₂ [M+H]⁺ m/z 291.07, found 290.95.

Step 2: tert-butyl (S)-((3-(6-chloro-2-(4,4-difluoroazepan-1-yl)nicotinamido)phenyl)(methyl)(oxo)-λ⁶-sulfaneylidene)carbamate: A mixture of 6-chloro-2-(4,4-difluoroazepan-1-yl)nicotinic acid (120 mg, 0.41 mmol) and tert-butyl (S)-((3-aminophenyl)(methyl)(oxo)-λ⁶-sulfaneylidene)carbamate (224 mg, 0.83 mmol) in pyridine (5 mL) was added POCl₃ (100 L) dropwise at 0° C. The reaction solution was stirred at 0° C. for 1 hour. After the reaction was complete, the resulting solution was diluted with water (20 mL) and extracted with DCM (20 mL×3). The combined organic phases were washed with brine, dried over sodium sulfate, concentrated under vacuum. The residue was purified by flash column chromatography on silica gel (PE/EtOAc=2/1) to give tert-butyl (S)-((3-(6-chloro-2-(4,4-difluoroazepan-1-yl)nicotinamido)phenyl)(methyl)(oxo)-λ⁶-sulfaneylidene)carbamate (110 mg, 49.5%) as a yellow oil. LCMS (ESI) calcd. for C₂₄H₂₉ClF₂N₄O₄SNa [M+H]⁺ m/z 565.16, found 565.10.

Step 3: tert-butyl (S)-((3-(6-(cyclopropylmethyl)-2-(4,4-difluoroazepan-1-yl)nicotinamido)phenyl)(methyl)(oxo)-λ⁶-sulfaneylidene)carbamate: A mixture tert-butyl (S)-((3-(6-chloro-2-(4,4-difluoroazepan-1-yl)nicotinamido)phenyl)(methyl)(oxo)-λ⁶-sulfaneylidene)carbamate (90 mg, 0.167 mmol), (cyclopropylmethyl)trifluoroborate potassium (83 mg, 0.52 mmol), cesium carbonate (163 mg, 0.5 mmol), Pd(OAc)₂ (15 mg, 0.07 mmol) and Ru-phos (24 mg, 0.052 mmol) in toluene/H₂O (10/1, 11 mL) was heated at 100° C. for 16 h under an atmosphere of N₂. The resulting solution was diluted with water (20 mL) and extracted with EtOAc (20 mL×3). The combined organic phases were washed with brine, dried over Na₂SO₄, concentrated under vacuum. The residue was purified by flash column chromatography on silica gel (PE/EtOAc=3/1) to give tert-butyl (S)-((3-(6-(cyclopropylmethyl)-2-(4,4-difluoroazepan-1-yl)nicotinamido)phenyl)(methyl)(oxo)-λ⁶-sulfaneylidene)carbamate (50 mg, 53.8%) as a yellow oil. LCMS (ESI) calcd. for C₂₈H₃₇F₂N₄O₄S [M+H]⁺ m/z 563.25, found 563.20.

Step 4: (S)-6-(cyclopropylmethyl)-2-(4,4-difluoroazepan-1-yl)-N-(3-(S-methylsulfonimidoyl)phenyl)nicotinamide: A solution of tert-butyl (S)-((3-(6-(cyclopropylmethyl)-2-(4,4-difluoroazepan-1-yl)nicotinamido)phenyl)(methyl)(oxo)-λ⁶-sulfaneylidene)carbamate (50 mg, 0.09 mmol) in DCM (3 mL) was added TFA (0.3 mL) at room temperature. The reaction mixture was stirred at room temperature for 2 hours. After the reaction was completed, the mixture was concentrated, and the residue was adjusted to pH=8-9 with saturated aqueous NaHCO₃. The aqueous solution was extracted with DCM (30 mL×3). The combined organic phases were washed with brine, dried over sodium sulfate, concentrated under vacuum. The residue was purified by prep-HPLC (Gemini 5 um C₁₈ column, 150*21.2 mm, eluting with 35% to 85% MeCN/H₂O containing 0.1% trifluoroacetic acid) to provide (S)-6-(cyclopropylmethyl)-2-(4,4-difluoroazepan-1-yl)-N-(3-(S-methylsulfonimidoyl)phenyl)nicotinamide (13.3 mg, 32.4%) as a white solid. ¹H NMR (400 MHz, DMSO-d₆) δ 10.64 (s, 1H), 8.34 (s, 1H), 7.91 (d, J=8.1 Hz, 1H), 7.73-7.47 (m, 3H), 6.70 (d, J=7.6 Hz, 1H), 4.18 (s, 1H), 3.63 (s, 2H), 3.42 (t, J=5.8 Hz, 2H), 3.04 (s, 3H), 2.53 (d, J=7.0 Hz, 2H), 2.32 (t, J=10.9 Hz, 2H), 2.03-1.90 (m, 2H), 1.89-1.76 (m, 2H), 1.12-1.02 (m, 1H), 0.58-0.39 (m, 2H), 0.26-0.17 (m, 2H). LCMS (ESI) calcd. for C₂₃H₂₉F₂N₄O₂S [M+H]⁺ m/z 463.20, found 463.10.

### Example 24

### (R)-6-(Cyclopropylmethyl)-2-(4,4-difluoroazepan-1-yl)-N-(3-(S-methylsulfonimidoyl)phenyl)nicotinamide

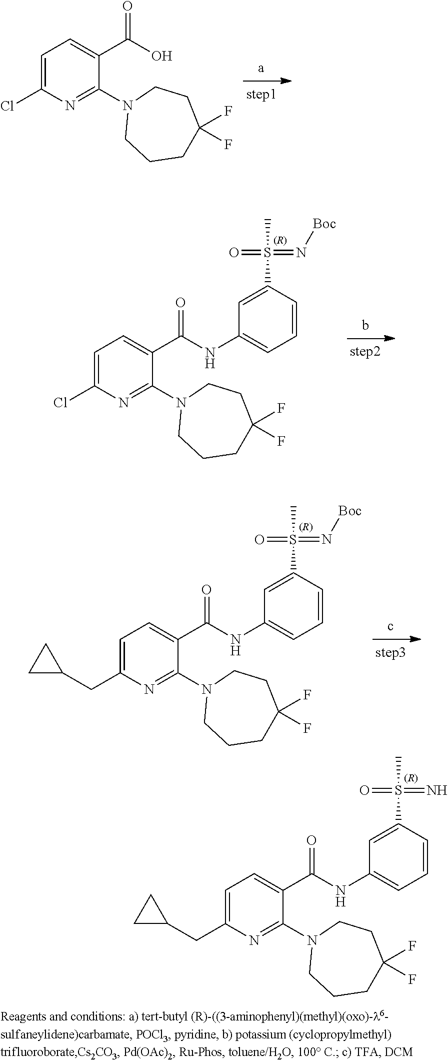

Step 1: tert-butyl (R)-((3-(6-chloro-2-(4,4-difluoroazepan-1-yl)nicotinamido)phenyl)(methyl)(oxo)-λ⁶-sulfaneylidene)carbamate: A mixture of 6-chloro-2-(4,4-difluoroazepan-1-yl)nicotinic acid (150 mg, 0.52 mmol) and tert-butyl (R)-((3-aminophenyl)(methyl)(oxo)-λ⁶-sulfaneylidene)carbamate (279 mg, 1.03 mmol) in pyridine (5 mL) was added POCl₃ (100 L) dropwise at 0° C. The reaction solution was stirred at 0° C. for 1 hour. After the reaction was completed, the resulting solution was diluted with water (20 mL) and extracted with DCM (20 mL×3). The combined organic phases were washed with brine, dried over sodium sulfate, concentrated under vacuum. The residue was purified by flash column chromatography on silica gel (PE/EtOAc=2/1) to give tert-butyl (R)-((3-(6-chloro-2-(4,4-difluoroazepan-1-yl)nicotinamido)phenyl)(methyl)(oxo)-λ⁶-sulfaneylidene)carbamate (140 mg, 49.8%) as a yellow oil. LCMS (ESI) calcd. for C₂₄H₂₉ClF₂N₄O₄SNa [M+Na]⁺ m/z 565.16, found 565.10.

Step 2: tert-butyl (R)-((3-(6-(cyclopropylmethyl)-2-(4,4-difluoroazepan-1-yl)nicotinamido)phenyl)(methyl)(oxo)-λ⁶-sulfaneylidene)carbamate: A mixture tert-butyl (R)-((3-(6-chloro-2-(4,4-difluoroazepan-1-yl)nicotinamido)phenyl)(methyl)(oxo)-λ⁶-sulfaneylidene)carbamate (140 mg, 0.26 mmol), (cyclopropylmethyl)trifluoroborate potassium (83 mg, 0.52 mmol), cesium carbonate (250 mg, 0.78 mmol), Pd(OAc)₂ (15 mg, 0.07 mmol) and Ru-phos (24 mg, 0.052 mmol) in toluene/H₂O (10/1, 11 mL) was heated at 100° C. for 16 h under an atmosphere of N₂. After the reaction was completed, the mixture was cooled to room temperature. The resulting solution was diluted with water (20 mL) and extracted with EtOAc (20 mL×3). The combined organic phases were washed with brine, dried over Na₂SO₄, concentrated under vacuum. The residue was purified by flash column chromatography on silica gel (PE/EtOAc=3/1) to give tert-butyl (R)-((3-(6-(cyclopropylmethyl)-2-(4,4-difluoroazepan-1-yl)nicotinamido)phenyl)(methyl)(oxo)-λ⁶-sulfaneylidene)carbamate (75 mg, 51.3%). LCMS (ESI) calcd. for C₂₈H₃₇F₂N₄O₄S [M+H]⁺ m/z 563.25, found 563.25.

Step 3: (R)-6-(cyclopropylmethyl)-2-(4,4-difluoroazepan-1-yl)-N-(3-(S-methylsulfonimidoyl)phenyl)nicotinamide: A solution of tert-butyl (R)-((3-(6-(cyclopropylmethyl)-2-(4,4-difluoroazepan-1-yl)nicotinamido)phenyl)(methyl)(oxo)-λ⁶-sulfaneylidene)carbamate (75 mg, 0.13 mmol) in DCM (3 mL) was added TFA (0.3 mL) at room temperature. The reaction mixture was stirred at room temperature for 2 hours. After the reaction was completed, the mixture was concentrated and the residue was adjusted to pH=8-9 with saturated aqueous NaHCO₃. The aqueous solution was extracted with DCM (30 mL×3). The combined organic phases were washed with brine, dried over sodium sulfate, concentrated under vacuum. The residue was purified by prep-HPLC (Gemini 5 um C₁₈ column, 150*21.2 mm, eluting with 35% to 85% MeCN/H₂O containing 0.1% trifluoroacetic acid) to provide (R)-6-(cyclopropylmethyl)-2-(4,4-difluoroazepan-1-yl)-N-(3-(S-methylsulfonimidoyl)phenyl)nicotinamide (26.2 mg, 39%) as a white solid. ¹H NMR (400 MHz, DMSO-d₆) δ 10.64 (s, 1H), 8.34 (s, 1H), 7.91 (d, J=8.1 Hz, 1H), 7.73-7.47 (m, 3H), 6.70 (d, J=7.6 Hz, 1H), 4.18 (s, 1H), 3.63 (s, 2H), 3.42 (t, J=5.8 Hz, 2H), 3.04 (s, 3H), 2.53 (d, J=7.0 Hz, 2H), 2.32 (t, J=10.9 Hz, 2H), 2.03-1.90 (m, 2H), 1.89-1.76 (m, 2H), 1.12-1.02 (m, 1H), 0.58-0.39 (m, 2H), 0.26-0.17 (m, 2H). LCMS (ESI) calcd. for C₂₃H₂₉F₂N₄O₂S [M+H]⁺ m/z 463.20, found 463.15.

### Example 25

### 6-Methyl-N-(3-(S-methylsulfonimidoyl)phenyl)-2-(6-azaspiro[2.5]octan-6-yl)-5-(trifluoromethyl)nicotinamide

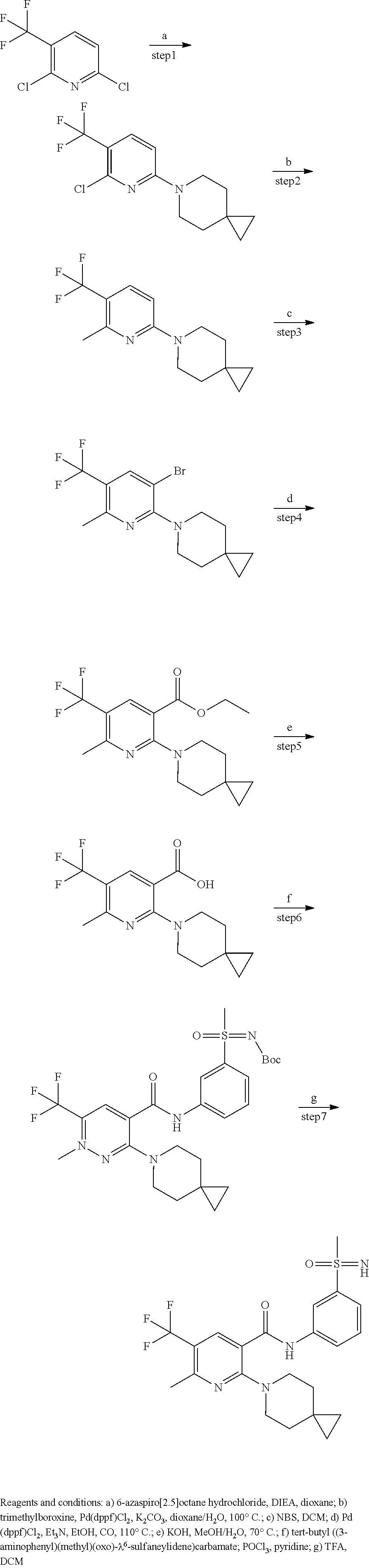

Step 1: 6-(6-chloro-5-(trifluoromethyl)pyridin-2-yl)-6-azaspiro[2.5]octane: A mixture of 2,6-dichloro-3-(trifluoromethyl)pyridine (3 g, 13.95 mmol) and 6-azaspiro[2.5]octane hydrochloride (2.06 g, 13.95 mmol) in 1,4-dioxane (50 mL) was added DIEA (3.60 g, 27.90 mmol) at room temperature. The reaction mixture was heated at 80° C. for 16 hours. After the reaction was completed, the mixture was cooled to room temperature, diluted with water (100 mL) and extracted with DCM (100 mL×3). The combined organic phases were washed with brine, dried over sodium sulfate, concentrated under vacuum. The residue was purified by flash column chromatography on silica gel (PE/EtOAc=20/1) to provide 6-(6-chloro-5-(trifluoromethyl)pyridin-2-yl)-6-azaspiro[2.5]octane (2.5 g, 61.6%) as a yellow oil. LCMS (ESI) calcd. for C₁₃H₁₅ClF₃N₂ [M+H]⁺ m/z 291.09, found 291.00.

Step 2: 6-(6-methyl-5-(trifluoromethyl)pyridin-2-yl)-6-azaspiro[2.5]octane: A mixture of 6-(6-chloro-5-(trifluoromethyl)pyridin-2-yl)-6-azaspiro[2.5]octane (2.5 g, 8.59 mmol), trimethylboroxine (10.78 g, 85.9 mmol), Pd(dppf)Cl₂ (628 mg, 0.86 mmol) and K₂CO₃ (3.55 g, 25.77 mmol) in dioxane/H₂O (4/1, 25 mL) was heated at 100° C. for 16 hours under an atmosphere of N₂. After cooling to ambient temperature, the mixture was filtered through celite and the filtrate was concentrated under vacuum. The residue was diluted with water and extracted with EtOAc. The combined organic phases were washed with water and brine, dried over sodium sulfate, concentrated under vacuum. The residue was purified by flash column chromatography on silica gel (PE/EtOAc=20/1) to give the 6-(6-methyl-5-(trifluoromethyl)pyridin-2-yl)-6-azaspiro[2.5]octane (1.9 g, 81.6%) as a yellow oil. LCMS (ESI) calcd. for C₁₄H₁₈F₃N₂ [M+H]⁺ m/z 271.14, found 271.10.

Step 3: 6-(3-bromo-6-methyl-5-(trifluoromethyl)pyridin-2-yl)-6-azaspiro[2.5]octane: To a solution of 6-(6-methyl-5-(trifluoromethyl)pyridin-2-yl)-6-azaspiro[2.5]octane (1.9 g, 7.01 mmol) in DCM (30 mL) was added NBS (1.37 g, 7.71 mmol). The mixture was stirred at room temperature for 2 hours. The resulting mixture was concentrated and directly purified by flash column chromatography on silica gel (PE/EtOAc=10/1) to give the 6-(3-bromo-6-methyl-5-(trifluoromethyl)pyridin-2-yl)-6-azaspiro[2.5]octane (1.5 g, 65.9%) as a yellow oil. LCMS (ESI) calcd. for C₁₄H₁₇BrF₃N₂ [M+H]⁺ m/z 351.05, found 351.00.

Step 4: ethyl 6-methyl-2-(6-azaspiro[2.5]octan-6-yl)-5-(trifluoromethyl)nicotinate: A mixture of 6-(3-bromo-6-methyl-5-(trifluoromethyl)pyridin-2-yl)-6-azaspiro[2.5]octane (1.5 g, 4.30 mmol), Pd(dppf)Cl₂ (157.16 mg, 0.21 mmol) and triethylamine (1.30 g, 12.9 mmol) in EtOH (10 mL) was heated at 110° C. under an atmosphere of CO (30 atm) for 16 hours in a high-pressure reactor. After cooling to ambient temperature, the mixture was filtered through celite and the filtrate was concentrated under vacuum. The residue was purified by flash column chromatography on silica gel (PE/EtOAc=10/1) to give ethyl 6-methyl-2-(6-azaspiro[2.5]octan-6-yl)-5-(trifluoromethyl)nicotinate (1.3 g, 94%) as a yellow solid. LCMS (ESI) calcd. for C₁₇H₂₂F₃N₂O₂ [M+H]⁺ m/z 343.17, found 343.15.

Step 5: 6-methyl-2-(6-azaspiro[2.5]octan-6-yl)-5-(trifluoromethyl)nicotinic acid: To a solution of ethyl 6-methyl-2-(6-azaspiro[2.5]octan-6-yl)-5-(trifluoromethyl)nicotinate (1.3 g, 3.79 mmol) in MeOH/H₂O (1/1, 40 mL) was added KOH (2.12 g, 37.9 mmol) at room temperature. The mixture was heated at 70° C. for 4 hours. After the reaction was completed, the mixture was cooled to room temperature and concentrated to remove MeOH. The aqueous phase was adjusted to pH=3-4 with 1N HCl and extracted with EtOAc (50 mL×3). The combined organic phases were washed with brine, dried with Na₂SO₄, and concentrated under reduced pressure to give 6-methyl-2-(6-azaspiro[2.5]octan-6-yl)-5-(trifluoromethyl)nicotinic acid (900 mg, 75.4%) as a yellow solid. LCMS (ESI) calcd. for C₁₅H₁₈F₃N₂O₂ [M+H]⁺ m/z 315.13, found 315.15.

Step 6: tert-butyl (methyl(3-(6-methyl-2-(6-azaspiro[2.5]octan-6-yl)-5-(trifluoromethyl)nicotinamido)phenyl)(oxo)-λ⁶-sulfaneylidene)carbamate: To a mixture of 6-methyl-2-(6-azaspiro[2.5]octan-6-yl)-5-(trifluoromethyl)nicotinic acid (250 mg, 0.7936 mmol) and tert-butyl ((3-aminophenyl)(methyl)(oxo)-λ⁶-sulfaneylidene)carbamate (322.62 mg, 1.1904 mmol) in pyridine (8 mL) was added POCl₃ (750 L) at room temperature.

The reaction mixture was stirred at room temperature for 2 hours. Then the mixture was diluted with water (10 mL) and extracted with DCM (10 mL×3). The combined organic phases were washed with brine, dried over sodium sulfate, concentrated under vacuum. The residue was purified by flash column chromatography on silica gel (PE/EtOAc=1/1) to give tert-butyl (methyl(3-(6-methyl-2-(6-azaspiro[2.5]octan-6-yl)-5-(trifluoromethyl)nicotinamido)phenyl)(oxo)-λ⁶-sulfaneylidene)carbamate (190 mg, 42.2%) as a yellow solid. LCMS (ESI) calcd. for C₂₇H₃₄F₃N₄O₄S [M+H]⁺ m/z 567.23, found 567.15.

Step 7: 6-methyl-N-(3-(S-methylsulfonimidoyl)phenyl)-2-(6-azaspiro[2.5]octan-6-yl)-5-(trifluoromethyl)nicotinamide: A solution of tert-butyl (methyl(3-(6-methyl-2-(6-azaspiro[2.5]octan-6-yl)-5-(trifluoromethyl)nicotinamido)phenyl)(oxo)-λ⁶-sulfaneylidene)carbamate (190 mg, 0.3351 mmol) in DCM (6 mL) was added TFA (2 mL). The reaction mixture was stirred at room temperature for 2 hours. After the reaction was completed, the solution was diluted with water (10 mL) and extracted with DCM (10 mL×3). The combined organic phases were washed with brine, dried over sodium sulfate and concentrated under vacuum. The residue was purified by prep-HPLC (Gemini 5 um C₁₈ column, 150×21.2 mm, eluting with 40% to 85% ACN-H₂O containing 0.1% formic acid) to afford (50 mg, 32%) as a white solid. ¹H NMR (400 MHz, DMSO-d₆) δ 10.77 (s, 1H), 8.34 (s, 1H), 7.97-7.86 (m, 2H), 7.65 (d, J=7.8 Hz, 1H), 7.58 (t, J=7.9 Hz, 1H), 3.64-3.52 (m, 4H), 3.07 (s, 3H), 2.50 (s, 3H), 1.41-1.32 (m, 4H), 0.31 (s, 4H). LCMS (ESI) calcd. for C₂₂H₂₆F₃N₄O₂S [M+H]⁺ m/z 467.18, found 467.10.

### Example 26

### (R)-5-Chloro-6-methyl-N-(3-(S-methylsulfonimidoyl)phenyl)-2-(6-azaspiro[2.5]octan-6-yl)nicotinamide

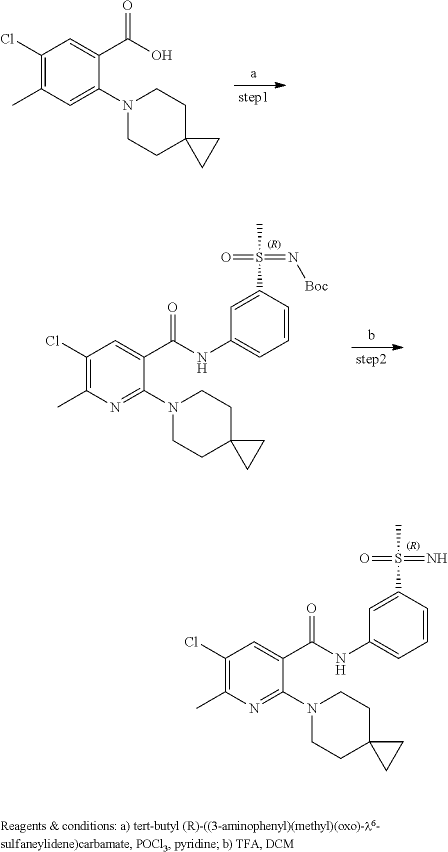

Step 1: tert-butyl (R)-((3-(5-chloro-6-methyl-2-(6-azaspiro[2.5]octan-6-yl)nicotinamido)phenyl)(methyl)(oxo)-16-sulfaneylidene)carbamate: A mixture of 5-chloro-6-methyl-2-(6-azaspiro[2.5]octan-6-yl)nicotinic acid (250 mg, 0.892 mmol) and tert-butyl (R)-((3-aminophenyl)(methyl)(oxo)-λ⁶-sulfaneylidene)carbamate (265 mg, 0.982 mmol) in pyridine (4 mL) was added POCl₃ (375 L) at 50° C. The reaction was monitored by LCMS. After the reaction was completed, the mixture was cooled to room temperature. The resulting solution was diluted with water (15 mL) and extracted with DCM (15 mL×3). The combined organic phases were washed with brine, dried over Na₂SO₄, concentrated under vacuum. The residue was purified by flash column chromatography on silica gel (PE/EtAOc=1/1) to give tert-butyl (R)-((3-(5-chloro-6-methyl-2-(6-azaspiro[2.5]octan-6-yl)nicotinamido)phenyl)(methyl)(oxo)-λ⁶-sulfaneylidene)carbamate (120 mg, 25.3%) as a yellow oil. LCMS (ESI) calcd. for C₂₆H₃₄ClN₄O₄S [M+H]⁺ m/z 533.20, found 533.15.

Step 2: (R)-5-chloro-6-methyl-N-(3-(S-methylsulfonimidoyl)phenyl)-2-(6-azaspiro[2.5]octan-6-yl)nicotinamide: A solution of tert-butyl (R)-((3-(5-chloro-6-methyl-2-(6-azaspiro[2.5]octan-6-yl)nicotinamido)phenyl)(methyl)(oxo)-λ⁶-sulfaneylidene)carbamate (120 mg, 0.225 mmol) in DCM (3 mL) was added TFA (1 mL) at room temperature. The reaction mixture was stirred at room temperature for 2 hours. After the reaction was completed, the solution was diluted with water (10 mL) and extracted with DCM (10 mL×3). The combined organic phases were washed with brine, dried over sodium sulfate, concentrated under vacuum. The residue was purified by prep-HPLC (Gemini 5 um C₁₈ column, 150×21.2 mm, eluting with 30% to 90% ACN-H₂O containing 0.1% formic acid) to obtain (R)-5-chloro-6-methyl-N-(3-(S-methylsulfonimidoyl)phenyl)-2-(6-azaspiro[2.5]octan-6-yl)nicotinamide (70 mg, 71.4%) as a white solid. ¹H NMR (400 MHz, DMSO-d₆, ppm) δ 10.82 (s, 1H), 8.41 (s, 1H), 7.89 (d, J=7.6 Hz, 1H), 7.80 (s, 1H), 7.67 (d, J=8 Hz, 1H), 7.62-7.58 (m, 1H), 3.36-3.33 (m, 4H), 3.11 (s, 3H), 2.46 (s, 3H), 1.38-1.36 (m, 4H), 0.29 (s, 4H). LCMS (ESI) calcd. for C₂₁H₂₆ClN₄O₂S [M+H]⁺ m/z 433.15, found 433.05.

### Example 27

### (S)-5-Chloro-6-methyl-N-(3-(S-methylsulfonimidoyl)phenyl)-2-(6-azaspiro[2.5]octan-6-yl)nicotinamide

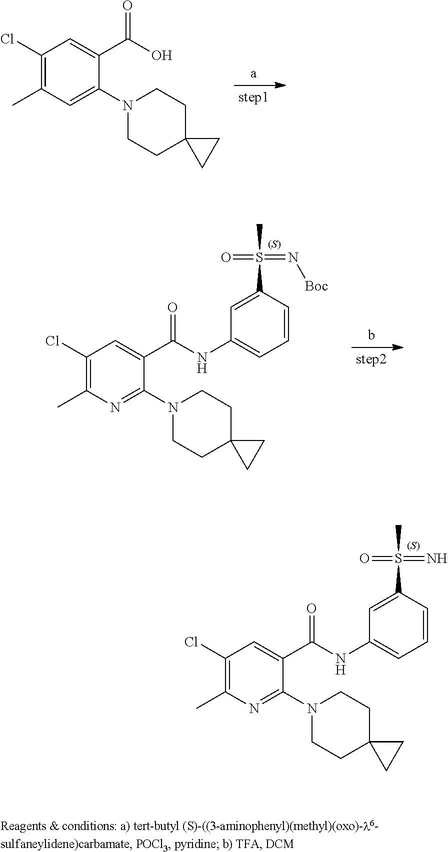

Step 1: tert-butyl (S)-((3-(5-chloro-6-methyl-2-(6-azaspiro[2.5]octan-6-yl)nicotinamido)phenyl)(methyl)(oxo)-λ⁶-sulfaneylidene)carbamate: A mixture of 5-chloro-6-methyl-2-(6-azaspiro[2.5]octan-6-yl)nicotinic acid (200 mg, 0.7143 mmol) and tert-butyl (S)-((3-aminophenyl)(methyl)(oxo)-λ⁶-sulfaneylidene)carbamate (232.29 mg, 0.8571 mmol) in pyridine (5 mL) was added POCl₃ (500 μL) dropwise at room temperature. The reaction was monitored by LCMS. After the reaction was completed, the resulting solution was diluted with water (20 mL) and extracted with DCM (20 mL×3). The combined organic phases were washed with brine, dried over sodium sulfate, concentrated under vacuum. The residue was purified by flash column chromatography on silica gel (PE/EtOAc=2/1) to give tert-butyl (S)-((3-(5-chloro-6-methyl-2-(6-azaspiro[2.5]octan-6-yl)nicotinamido)phenyl)(methyl)(oxo)-λ⁶-sulfaneylidene)carbamate (120 mg, 31.5%) as a white solid. LCMS (ESI) calcd. for C₂₆H₃₄ClN₄O₄S [M+H]⁺ m/z 533.20, found 533.15.

Step 2: (S)-5-chloro-6-methyl-N-(3-(S-methylsulfonimidoyl)phenyl)-2-(6-azaspiro[2.5]octan-6-yl)nicotinamide: A solution of tert-butyl (S)-((3-(5-chloro-6-methyl-2-(6-azaspiro[2.5]octan-6-yl)nicotinamido)phenyl)(methyl)(oxo)-λ⁶-sulfaneylidene)carbamate (120 mg, 0.2169 mmol) in DCM (3 mL) was added TFA (1 mL) at room temperature. The reaction mixture was stirred at room temperature for 2 hours. The reaction was monitored by LCMS. After the reaction was completed, the mixture was diluted with water (10 mL) and extracted with DCM (10 mL×3). The combined organic phases were washed with brine, dried over sodium sulfate, concentrated under vacuum. The residue was purified by prep-HPLC (Gemini 5 um C₁₈ column, 150×21.2 mm, eluting with 45% to 95% ACN-H₂O containing 0.1% formic acid) to provide (S)-5-chloro-6-methyl-N-(3-(S-methylsulfonimidoyl)phenyl)-2-(6-azaspiro[2.5]octan-6-yl)nicotinamide (42 mg, 44.72%) as a white solid. ¹H NMR (400 MHz, DMSO-d₆, ppm) δ 10.81 (s, 1H), 8.39 (s, 1H), 7.88 (d, J=8.0 Hz, 1H), 7.80 (s, 1H), 7.65 (d, J=7.8 Hz, 1H), 7.59 (t, J=7.9 Hz, 1H), 3.36 (d, J=5.2 Hz, 4H), 3.06 (s, 3H), 2.47 (s, 3H), 1.43-1.31 (m, 4H), 0.29 (s, 4H). LCMS (ESI) calcd. for C₂₁H₂₆ClN₄O₂S [M+H]⁺ m/z 433.15, found 433.10.

### Example 28

### (R)-3-(4,4-Difluoroazepan-1-yl)-5-methyl-N-(3-(S-methylsulfonimidoyl)phenyl)-6-(trifluoromethyl)pyridazine-4-carboxamide

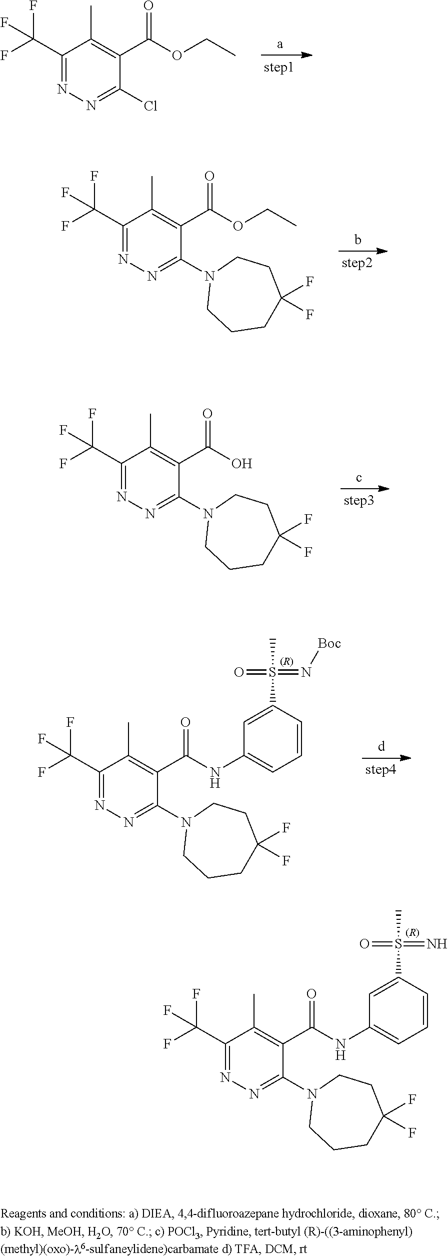

Step 1: ethyl 3-(4,4-difluoroazepan-1-yl)-5-methyl-6-(trifluoromethyl)pyridazine-4-carboxylate: A solution of ethyl 3-chloro-5-methyl-6-(trifluoromethyl)pyridazine-4-carboxylate (800 mg, 2.99 mmol), 4,4-difluoroazepane hydrochloride (613 mg, 3.58 mmol) and DIEA (1155 mg, 8.96 mmol) in dioxane (10 mL) was heated at 80° C. for 1 h. LCMS showed the reaction was completed. The mixture concentrated under vacuum and purified by flash column chromatography on silica gel (PE/EtOAc=3/1) to provide ethyl 3-(4,4-difluoroazepan-1-yl)-5-methyl-6-(trifluoromethyl)pyridazine-4-carboxylate (800 mg, 73.1%) as a yellow solid. LCMS (ESI) calcd. for C₁₅H₁₉F₅N₃O₂ [M+H]⁺ m/z 368.14, found 367.95.

Step 2: 3-(4,4-difluoroazepan-1-yl)-5-methyl-6-(trifluoromethyl)pyridazine-4-carboxylic acid: To a solution of ethyl 3-(4,4-difluoroazepan-1-yl)-5-methyl-6-(trifluoromethyl)pyridazine-4-carboxylate (800 mg, 2.48 mmol) in MeOH/H₂O (1/1, 10 mL) was added KOH (872 mg, 21.80 mmol) at room temperature. The mixture was heated at 70° C. for 5 hours. After the reaction was completed, the mixture was concentrated to remove most MeOH. The aqueous phase was adjusted to pH=3-4 with 1N HCl then extracted with EtOAc (20 mL×3). The combined organic phases were washed with brine, dried with Na₂SO₄, and concentrated under reduced pressure to afford 3-(4,4-difluoroazepan-1-yl)-5-methyl-6-(trifluoromethyl)pyridazine-4-carboxylic acid (600 mg, 81.2%) as a yellow solid. LCMS (ESI) calcd. for C₁₃H₁₅F₅N₃O₂ [M+H]⁺ m/z 340.11, found 340.00.

Step 3: tert-butyl (R)-((3-(3-(4,4-difluoroazepan-1-yl)-5-methyl-6-(trifluoromethyl)pyridazine-4-carboxamido)phenyl)(methyl)(oxo)-λ⁶-sulfaneylidene)carbamate: To a solution of 3-(4,4-difluoroazepan-1-yl)-5-methyl-6-(trifluoromethyl)pyridazine-4-carboxylic acid (200 mg, 0.59 mmol) and tert-butyl (R)-((3-aminophenyl)(methyl)(oxo)-λ⁶-sulfaneylidene)carbamate (175 mg, 0.65 mmol) in pyridine (10 mL) was added POCl₃ (100 L) dropwise at 50° C. The mixture was heated at 50° C. for 2 hours. LCMS showed the reaction was completed. The mixture was quenched with water (40 mL) and extracted with EtOAc (40 mL×3). The combined organic phases were washed with brine, dried over sodium sulfate, concentrated under vacuum. The residue was purified by flash column chromatography on silica gel (PE/EtOAc=1/3) to provide tert-butyl (R)-((3-(3-(4,4-difluoroazepan-1-yl)-5-methyl-6-(trifluoromethyl)pyridazine-4-carboxamido)phenyl)(methyl)(oxo)-λ⁶-sulfaneylidene)carbamate (90 mg, 25.9%) as a yellow solid. LCMS (ESI) calcd. for C₂₀H₂₃F₅N₅O₂S [M-Boc+H]⁺ m/z 492.15, found 492.15.

Step 4: (R)-3-(4,4-difluoroazepan-1-yl)-5-methyl-N-(3-(S-methylsulfonimidoyl)phenyl)-6-(trifluoromethyl)pyridazine-4-carboxamide: To a solution of tert-butyl (R)-((3-(3-(4,4-difluoroazepan-1-yl)-5-methyl-6-(trifluoromethyl)pyridazine-4-carboxamido)phenyl)(methyl)(oxo)-λ⁶-sulfaneylidene)carbamate (90 mg, 0.15 mmol) in DCM (1.5 mL) was added TFA (0.5 mL) at room temperature. The mixture was stirred at room temperature for 1 hour. LCMS showed the reaction was completed. The final mixture was concentrated in vacuum and purified by prep-HPLC (Gemini 5 um C₁₈ column, 150×21.2 mm, eluting with 30% to 90% ACN-H₂O containing 0.1% ammonium hydroxide) to afford (R)-3-(4,4-difluoroazepan-1-yl)-5-methyl-N-(3-(S-methylsulfonimidoyl)phenyl)-6-(trifluoromethyl)pyridazine-4-carboxamide (15 mg, 20.3%) as a yellow solid. ¹H NMR (400 MHz, DMSO-d₆) δ 11.11 (s, 1H), 8.27 (s, 1H), 7.88 (d, J=8.1 Hz, 1H), 7.72 (d, J=7.8 Hz, 1H), 7.63 (t, J=7.9 Hz, 1H), 4.28 (s, 1H), 3.85 (s, 2H), 3.69 (t, J=5.9 Hz, 2H), 3.08 (s, 3H), 2.33 (s, 3H), 2.11-1.99 (m, 2H), 1.93-1.81 (m, 2H), 1.23 (s, 2H). LCMS (ESI) calcd. for C₂₀H₂₃F₅N₅O₂S [M+H]⁺ m/z 492.15, found 492.10.

### Example 29

### (S)-3-(4,4-Difluoroazepan-1-yl)-5-methyl-N-(3-(S-methylsulfonimidoyl)phenyl)-6-(trifluoromethyl)pyridazine-4-carboxamide

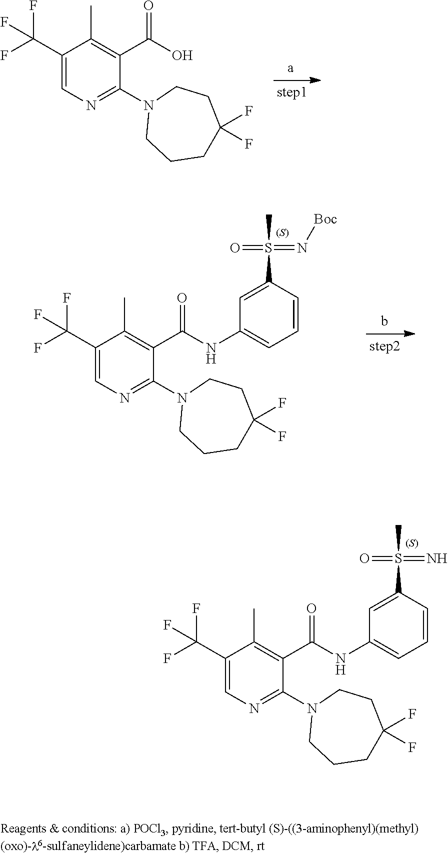

Step 1: tert-butyl (S)-((3-(3-(4,4-difluoroazepan-1-yl)-5-methyl-6-(trifluoromethyl)pyridazine-4-carboxamido)phenyl)(methyl)(oxo)-λ⁶-sulfaneylidene)carbamate: A mixture of 3-(4,4-difluoroazepan-1-yl)-5-methyl-6-(trifluoromethyl)pyridazine-4-carboxylic acid (200 mg, 0.59 mmol) and tert-butyl (S)-((3-aminophenyl)(methyl)(oxo)-16-sulfaneylidene)carbamate (191.16 mg, 0.708 mmol) in pyridine (3.2 mL) was added POCl₃ (400 L) dropwise at 50° C. The reaction was monitored by LCMS. After the reaction was completed, the mixture was cooled to room temperature. The resulting solution was diluted with water (20 mL) and extracted with DCM (20 mL×3). The combined organic phases were washed with brine, dried over sodium sulfate, concentrated under vacuum. The residue was purified by flash column chromatography on silica gel (PE/EtOAc=1/1) to give tert-butyl (S)-((3-(3-(4,4-difluoroazepan-1-yl)-5-methyl-6-(trifluoromethyl)pyridazine-4-carboxamido)phenyl)(methyl)(oxo)-λ⁶-sulfaneylidene)carbamate (120 mg, 34.2%) as a yellow solid. LCMS (ESI) calcd. for C₂₅H₃₁F₅N₅O₄S [M+H]⁺ m/z 592.19, found 592.15.

Step 2: (S)-3-(4,4-difluoroazepan-1-yl)-5-methyl-N-(3-(S-methylsulfonimidoyl)phenyl)-6-(trifluoromethyl)pyridazine-4-carboxamide: A solution of give tert-butyl (S)-((3-(3-(4,4-difluoroazepan-1-yl)-5-methyl-6-(trifluoromethyl)pyridazine-4-carboxamido)phenyl)(methyl)(oxo)-λ⁶-sulfaneylidene)carbamate (120 mg, 0.203 mmol) in DCM (3 mL) was added TFA (1 mL) at room temperature. The reaction mixture was stirred at room temperature for 2 hours. After the reaction was completed, the mixture was diluted with water (10 mL) and extracted with DCM (10 mL×3). The combined organic phases were washed with brine, dried over sodium sulfate, concentrated under vacuum. The residue was purified by prep-HPLC (Gemini 5 um C₁₈ column, 150×21.2 mm, eluting with 30% to 90% ACN-H₂O containing 0.1% formic acid) to obtain (S)-3-(4,4-difluoroazepan-1-yl)-5-methyl-N-(3-(S-methylsulfonimidoyl)phenyl)-6-(trifluoromethyl)pyridazine-4-carboxamide (20 mg, 20%) as a white solid. ¹H NMR (400 MHz, DMSO-d₆) δ 11.12 (s, 1H), 8.27 (s, 1H), 7.88 (d, J=8.1 Hz, 1H), 7.73 (d, J=7.9 Hz, 1H), 7.63 (t, J=7.9 Hz, 1H), 3.85 (s, 2H), 3.69 (t, J=5.8 Hz, 2H), 3.08 (s, 3H), 2.37 (d, J=16.1 Hz, 2H), 2.33 (s, 3H), 2.12-2.02 (m, 2H), 1.92-1.85 (m, 2H). LCMS (ESI) calcd. for C₂₀H₂₃F₅N₅O₂S [M+H]⁺ m/z 492.15, found 492.00.

### Example 30

### (S)-6-(3-Buten-1-yl)-2-(4,4-difluoroazepan-1-yl)-N-(3-(S-methylsulfonimidoyl)phenyl)nicotinamide

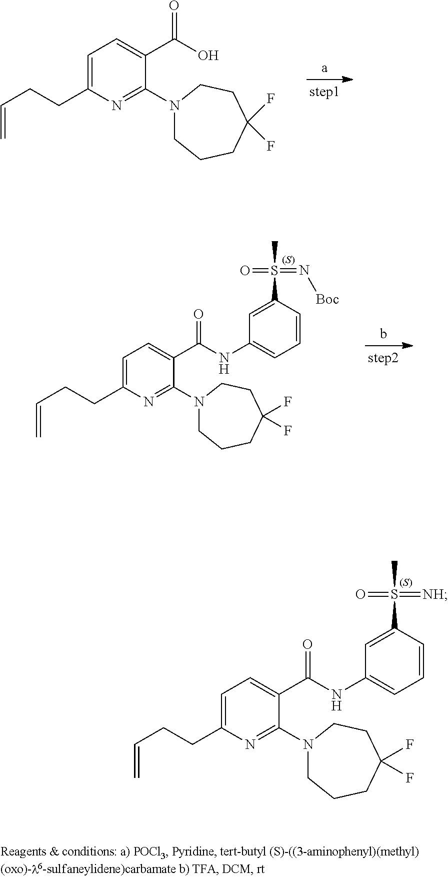

Step 1: tert-butyl (S)-((3-(6-(3-buten-1-yl)-2-(4,4-difluoroazepan-1-yl)nicotinamido)phenyl)(methyl)(oxo)-λ⁶-sulfaneylidene)carbamate: A mixture of 6-(3-buten-1-yl)-2-(4,4-difluoroazepan-1-yl)nicotinic acid (175 mg, 0.564 mmol) and tert-butyl (S)-((3-aminophenyl)(methyl)(oxo)-λ⁶-sulfaneylidene)carbamate (168 mg, 0.620 mmol) in pyridine (4 mL) was added POCl₃ (250 L) at 50° C. After the reaction was completed, the mixture was cooled to room temperature. The resulting solution was diluted with water (15 mL) and extracted with DCM (15 mL×3). The combined organic phases were washed with brine, dried over Na₂SO₄, concentrated under vacuum. The residue was purified by flash column chromatography on silica gel (PE/EtAOc=1/1) to give tert-butyl (S)-((3-(6-(3-buten-1-yl)-2-(4,4-difluoroazepan-1-yl)nicotinamido)phenyl)(methyl)(oxo)-λ⁶-sulfaneylidene)carbamate (80 mg, 25.15%) as a yellow oil. LCMS (ESI) calcd. for C₂₈H₃₇F₂N₄O₄S [M+H]⁺ m/z 563.25, found 563.20.

Step 2: (S)-6-(3-buten-1-yl)-2-(4,4-difluoroazepan-1-yl)-N-(3-(S-methylsulfonimidoyl)phenyl)nicotinamide: A solution of tert-butyl (S)-((3-(6-(3-buten-1-yl)-2-(4,4-difluoroazepan-1-yl)nicotinamido)phenyl)(methyl)(oxo)-λ⁶-sulfaneylidene)carbamate (80 mg, 0.143 mmol) in DCM (3 mL) was added TFA (1 mL) at room temperature. The reaction mixture was stirred at room temperature for 2 hours. After the reaction was completed, the solution was diluted with water (10 mL) and extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 30% to 90% ACN-H₂O containing 0.1% formic acid) to obtain (S)-6-(3-buten-1-yl)-2-(4,4-difluoroazepan-1-yl)-N-(3-(S-methylsulfonimidoyl)phenyl)nicotinamide (23 mg, 35%) as a white solid. ¹H NMR (400 MHz, DMSO-d₆) δ 10.68 (s, 1H), 8.37 (s, 1H), 7.91 (d, J=7.6 Hz, 1H), 7.66-7.56 (m, 2H), 6.65 (d, J=8 Hz, 1H), 5.92-5.82 (m, 1H), 5.07-4.95 (m, 2H), 3.64-3.62 (m, 2H), 3.43-3.40 (m, 2H), 3.14 (s, 3H), 2.74-2.67 (m, 2H), 2.45-2.43 (m, 2H), 2.34-2.32 (s, 2H), 1.93-1.84 (m, 4H). LCMS (ESI) calcd. for C₂₃H₂₉F₂N₄O₂S [M+H]⁺ m/z 463.20, found 463.10.

### Example 31

### 3-(4,4-Difluoroazepan-1-yl)-5-methyl-N-(pyridazin-4-yl)-6-(trifluoromethyl)pyridazine-4-carboxamide

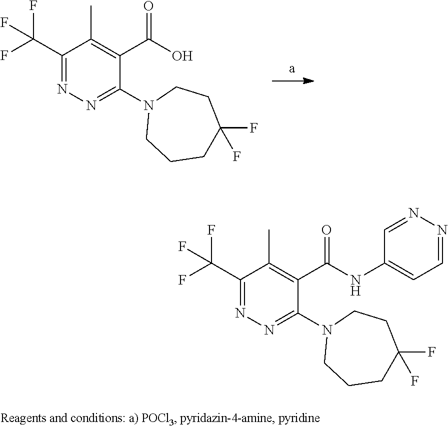 [SMILES: Cc1c(C(F)(F)F)nnc(N2CCCC(F)(F)CC2)c1C(=O)O ; Cc1c(C(F)(F)F)nnc(N2CCCC(F)(F)CC2)c1C(=O)Nc1ccnnc1]

A mixture of 3-(4,4-difluoroazepan-1-yl)-5-methyl-6-(trifluoromethyl)pyridazine-4-carboxylic acid (150 mg, 0.44 mmol) and pyridazin-4-amine (84 mg, 0.88 mmol) in pyridine (6 mL) was added POCl₃ (150 μL) dropwise at room temperature. The reaction was stirred at room temperature for 2 hours. After the reaction was completed, the resulting solution was diluted with water (40 mL) and extracted with DCM (30 mL×3). The combined organic phases were washed with brine, dried over sodium sulfate, concentrated under vacuum. The residue was first purified by flash column chromatography on silica gel (DCM/MeOH=20/1) then further purified by prep-HPLC (Gemini 5 um C₁₈ column, 150×21.2 mm, eluting with 35% to 75% ACN-H₂O containing 0.1% TFA) to give 3-(4,4-difluoroazepan-1-yl)-5-methyl-N-(pyridazin-4-yl)-6-(trifluoromethyl)pyridazine-4-carboxamide (25.2 mg, 13%) as a white solid. LCMS (ESI) calcd. for C₁₇H₁₈F₅N₆O [M+H]⁺ m/z 417.15, found 417.00. ¹H NMR (400 MHz, DMSO-d₆) δ 11.51 (s, 1H), 9.31 (d, J=2.3 Hz, 1H), 9.18 (d, J=5.9 Hz, 1H), 8.07 (dd, J=5.8, 2.7 Hz, 1H), 3.83 (s, 2H), 3.61 (t, J=5.9 Hz, 2H), 2.37 (d, J=15.7 Hz, 2H), 2.32 (s, 3H), 2.16-1.94 (m, 2H), 1.94-1.79 (m, 2H).

### Example 32

### 4-(4,4-Difluoroazepan-1-yl)-N-(pyridazin-4-yl)-6-(trifluoromethyl)pyridazine-3-carboxamide

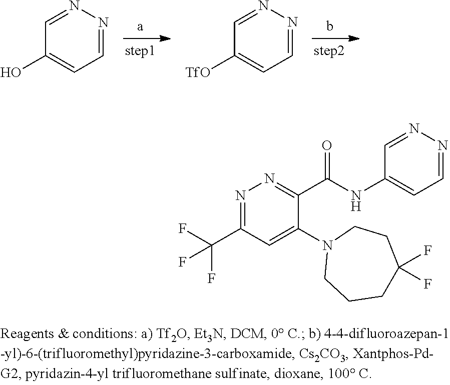 [SMILES: Oc1ccnnc1 ; O=S(=O)(Oc1ccnnc1)C(F)(F)F ; O=C(Nc1ccnnc1)c1nnc(C(F)(F)F)cc1N1CCCC(F)(F)CC1]

Step 1: pyridazin-4-yl trifluoromethanesulfonate: To a solution of pyridazin-4-ol (300 mg, 3.12 mmol) and Et₃N (630 mg, 6.24 mmol) in DCM (20 mL) was added Tf₂O (1056 mg, 3.74 mmol) at 0° C. The mixture was stirred at the same temperature for 30 minutes. The mixture was quenched with water (20 mL) and extracted with DCM (20 mL×2). The combined organic phases were washed with brine, dried over sodium sulfate, concentrated under vacuum. The residue was purified by flash column chromatography on silica gel (PE/EtOAc=3/1) to provide pyridazin-4-yl trifluoromethane sulfinate (100 mg, 14% yield) as a yellow oil. ¹H NMR (400 MHz, DMSO-d₆) δ 8.81 (d, J=8.5 Hz, 1H), 8.17 (d, J=2.7 Hz, 1H), 6.63 (dd, J=8.6, 2.8 Hz, 1H).

Step 2: 4-(4,4-difluoroazepan-1-yl)-N-(pyridazin-4-yl)-6-(trifluoromethyl)pyridazine-3-carboxamide: To a solution of 4-(4,4-difluoroazepan-1-yl)-6-(trifluoromethyl) pyridazine-3-carboxamide (100 mg, 0.31 mmol), pyridazin-4-yl trifluoromethane sulfinate (100 mg, 0.46 mmol) and Cs₂CO₃ (202 mg, 0.62 mmol) in 1,4-dioxane (5 mL) was added Xantphos-Pd-G2 (53 mg, 0.06 mmol). Then the mixture was heated at 100° C. for 16 hours under an atmosphere of N₂. The resulting mixture was filtered through celite. The filtrate was diluted with water (20 mL) and extracted with EtOAc (20 mL×3). The combined organic phases were washed with brine, dried over sodium sulfate, concentrated under vacuum. The residue was first purified by flash column chromatography on silica gel (PE/EtOAc=1/1), then further purified by prep-HPLC (Gemini 5 um C₁₈ column, 150×21.2 mm, eluting with 30% to 90% ACN-H₂O containing 0.1% formic acid) to afford 4-(4,4-difluoroazepan-1-yl)-N-(pyridazin-4-yl)-6-(trifluoromethyl)pyridazine-3-carboxamide (7 mg, 6%) as a white solid. ¹H NMR (400 MHz, DMSO-d₆) δ 11.68 (s, 1H), 9.51 (d, J=2.2 Hz, 1H), 9.13 (d, J=5.9 Hz, 1H), 8.10 (dd, J=5.8, 2.5 Hz, 1H), 7.49 (s, 1H), 3.71-3.60 (m, 2H), 3.49 (t, J=5.6 Hz, 2H), 2.32 (s, 2H), 2.04 (t, J=12.4 Hz, 2H), 1.90 (d, J=4.9 Hz, 2H). LCMS (ESI) calcd. for C₁₆H₁₆F₅N₆O [M+H]⁺ m/z 403.12, found 402.95.

### Example 33

### (R)-2-(4,4-Difluoroazepan-1-yl)-4-methyl-N-(3-(S-methylsulfonimidoyl)phenyl)-6-(trifluoromethyl)nicotinamide

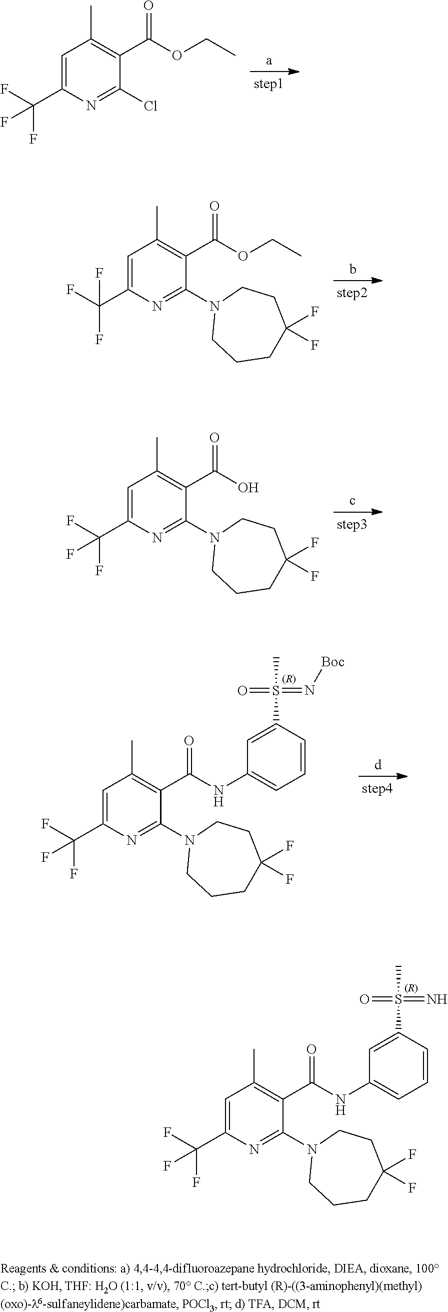

Step 1: ethyl 2-(4,4-difluoroazepan-1-yl)-4-methyl-6-(trifluoromethyl)nicotinate: A solution of ethyl 2-chloro-4-methyl-6-(trifluoromethyl)nicotinate (prepared by a known method WO2019166822) (800 mg, 2.9962 mmol), 4,4-difluoroazepane hydrochloride (1.03 g, 5.99 mmol) and DIEA (1.16 g, 8.99 mmol) in dioxane (12 mL) was heated at 100° C. for 4 hours. LCMS showed the reaction was completed. The mixture concentrated under vacuum and purified by flash column chromatography on silica gel (PE/EtOAc=3/1) to provide ethyl 2-(4,4-difluoroazepan-1-yl)-4-methyl-6-(trifluoromethyl)nicotinate (710 mg, 64.74%) as a yellow solid. LCMS (ESI) calcd. for C₁₆H₂₀F₅N₂O₂ [M+H]⁺ m/z 367.15, found 367.10.

Step 2: 2-(4,4-difluoroazepan-1-yl)-4-methyl-6-(trifluoromethyl)nicotinic acid: To a solution of ethyl 2-(4,4-difluoroazepan-1-yl)-4-methyl-6-(trifluoromethyl)nicotinate (710 mg, 1.94 mmol) in THF/H₂O (1/1, 10 mL) was added KOH (1.09 g, 19.40 mmol) at room temperature. The mixture was heated at 70° C. for 5 hours. After the reaction was completed, the mixture was concentrated to remove most THF. The aqueous phase was adjusted to pH=3-4 with 1N HCl then extracted with EtOAc (20 mL×3). The combined organic phases were washed with brine, dried with Na₂SO₄, and concentrated under reduced pressure to afford 2-(4,4-difluoroazepan-1-yl)-4-methyl-6-(trifluoromethyl)nicotinic acid (540 mg, 82.4%) as a yellow solid. LCMS (ESI) calcd. for C₁₄H₁₆F₅N₂O₂ [M+H]⁺ m/z 339.12, found 339.05.

Step 3: tert-butyl (R)-((3-(2-(4,4-difluoroazepan-1-yl)-4-methyl-6 (trifluoromethyl)nicotinamido)phenyl)(methyl)(oxo)-λ⁶-sulfaneylidene)carbamate: A mixture of 2-(4,4-difluoroazepan-1-yl)-4-methyl-6-(trifluoromethyl)nicotinic acid (100 mg, 0.2958 mmol) and tert-butyl (R)-((3-aminophenyl)(methyl)(oxo)-λ⁶-sulfaneylidene)carbamate (96.21 mg, 0.3550 mmol) in pyridine (3 mL) was added POCl₃ (100 L) dropwise at room temperature. The reaction solution was stirred at room temperature for 1 hour. After the reaction was completed, the resulting solution was diluted with water (20 mL) and extracted with DCM (20 mL×3). The combined organic phases were washed with brine, dried over sodium sulfate, concentrated under vacuum. The residue was purified by flash column chromatography on silica gel (PE/EtOAc=2/1) to give tert-butyl (R)-((3-(2-(4,4-difluoroazepan-1-yl)-4-methyl-6-(trifluoromethyl)nicotinamido)phenyl)(methyl)(oxo)-λ⁶-sulfaneylidene)carbamate (50 mg, 29%) as a yellow solid. LCMS (ESI) calcd. for C₂₆H₃₂F₅N₄O₄S [M+H]⁺ m/z 591.22, found 591.15.

Step 4: (R)-2-(4,4-difluoroazepan-1-yl)-4-methyl-N-(3-(S-methylsulfonimidoyl)phenyl)-6-(trifluoromethyl)nicotinamide: A solution of tert-butyl (R)-((3-(2-(4,4-difluoroazepan-1-yl)-4-methyl-6-(trifluoromethyl) nicotinamido)phenyl)(methyl)(oxo)-λ⁶-sulfaneylidene)carbamate (50 mg, 0.0846 mmol) in DCM (3 mL) was added TFA (1 mL) at room temperature. The reaction mixture was stirred at room temperature for 2 hours. The reaction was monitored by LCMS. After the reaction was completed, the mixture was diluted with water (10 mL) and extracted with DCM (10 mL×3). The combined organic phases were washed with brine, dried over sodium sulfate, concentrated under vacuum. The residue was purified by prep-HPLC (Gemini 5 um C₁₈ column, 150×21.2 mm, eluting with 50% to 55% ACN-H₂O containing 0.1% formic acid) to provide (R)-2-(4,4-difluoroazepan-1-yl)-4-methyl-N-(3-(S-methylsulfonimidoyl)phenyl)-6-(trifluoromethyl)nicotinamide (14.4 mg, 34.7%) as a white solid. ¹H NMR (400 MHz, DMSO-d₆) δ 10.90 (s, 1H), 8.33 (s, 1H), 7.90 (d, J=8.3 Hz, 1H), 7.69 (d, J=7.9 Hz, 1H), 7.61 (t, J=7.9 Hz, 1H), 7.16 (s, 1H), 3.68-3.63 (m, 2H), 3.58 (t, J=6.0 Hz, 2H), 3.10 (s, 3H), 2.36-2.21 (m, 5H), 2.06-1.95 (m, 2H), 1.88-1.75 (m, 2H). LCMS (ESI) calcd. for C₂₁H₂₄F₅N₄O₂S [M+H]⁺ m/z 491.16, found 491.00.

### Example 34

### (S)-2-(4,4-Difluoroazepan-1-yl)-4-methyl-N-(3-(S-methylsulfonimidoyl)phenyl)-6-(trifluoromethyl)nicotinamide

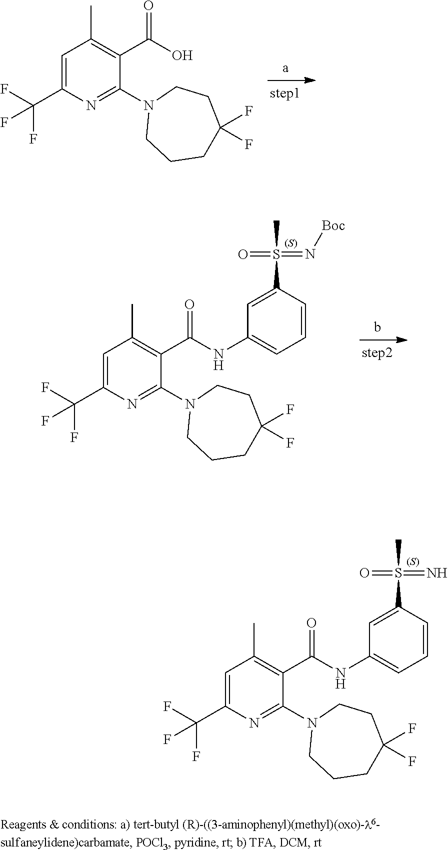

Step 1: tert-butyl (S)-((3-(2-(4,4-difluoroazepan-1-yl)-4-methyl-6-(trifluoromethyl)nicotinamido)phenyl)(methyl)(oxo)-16-sulfaneylidene)carbamate: A mixture of 2-(4,4-difluoroazepan-1-yl)-4-methyl-6-(trifluoromethyl)nicotinic acid (150 mg, 0.4438 mmol) and tert-butyl (S)-((3-aminophenyl)(methyl)(oxo)-λ⁶-sulfaneylidene)carbamate (144.32 mg, 0.5325 mmol) in pyridine (3 mL) was added POCl₃ (100 L) dropwise at room temperature. The reaction was monitored by LCMS. After the reaction was completed, the resulting solution was diluted with water (20 mL) and extracted with DCM (20 mL×3). The combined organic phases were washed with brine, dried over sodium sulfate, concentrated under vacuum. The residue was purified by flash column chromatography on silica gel (PE/EtOAc=2/1) to give tert-butyl (S)-((3-(2-(4,4-difluoroazepan-1-yl)-4-methyl-6-(trifluoromethyl)nicotinamido)phenyl)(methyl)(oxo)-λ⁶sulfaneylidene)carbamate (50 mg, 19.06%) as a yellow solid. LCMS (ESI) calcd. for C₂₆H₃₂F₅N₄O₄S [M+H]⁺ m/z 591.22, found 591.15.

Step 2: (S)-2-(4,4-difluoroazepan-1-yl)-4-methyl-N-(3-(S-methylsulfonimidoyl)phenyl)-6-(trifluoromethyl)nicotinamide: A solution of tert-butyl (S)-((3-(2-(4,4-difluoroazepan-1-yl)-4-methyl-6-(trifluoromethyl)nicotinamido)phenyl)(methyl)(oxo)-λ⁶-sulfaneylidene)carbamate (50 mg, 0.0846 mmol) in DCM (3 mL) was added TFA (1 mL) at room temperature. The reaction mixture was stirred at room temperature for 2 hours. The reaction was monitored by LCMS. After the reaction was completed, the mixture was diluted with water (10 mL) and extracted with DCM (10 mL×3). The combined organic phases were washed with brine, dried over sodium sulfate, concentrated under vacuum. The residue was purified by prep-HPLC (Gemini 5 um C₁₈ column, 150×21.2 mm, eluting with 50% to 55% ACN-H₂O containing 0.1% formic acid) to provide (S)-2-(4,4-difluoroazepan-1-yl)-4-methyl-N-(3-(S-methylsulfonimidoyl)phenyl)-6-(trifluoromethyl)nicotinamide (20.6 mg, 49.3%) as a white solid. ¹H NMR (400 MHz, DMSO-d₆) δ 10.90 (s, 1H), 8.33 (s, 1H), 7.90 (d, J=8.0 Hz, 1H), 7.72-7.57 (m, 2H), 7.16 (s, 1H), 3.65 (d, J=7.9 Hz, 2H), 3.58 (t, J=6.0 Hz, 2H), 3.09 (s, 3H), 2.36-2.20 (m, 5H), 2.08-1.94 (m, 2H), 1.87-1.77 (m, 2H). LCMS (ESI) calcd. for C₂₁H₂₄F₅N₄O₂S [M+H]⁺ m/z 491.16, found 491.00.

### Example 35

### (R)-6-(But-3-en-1-yl)-2-(4,4-difluoroazepan-1-yl)-N-(3-(S-methylsulfonimidoyl)phenyl)nicotinamide

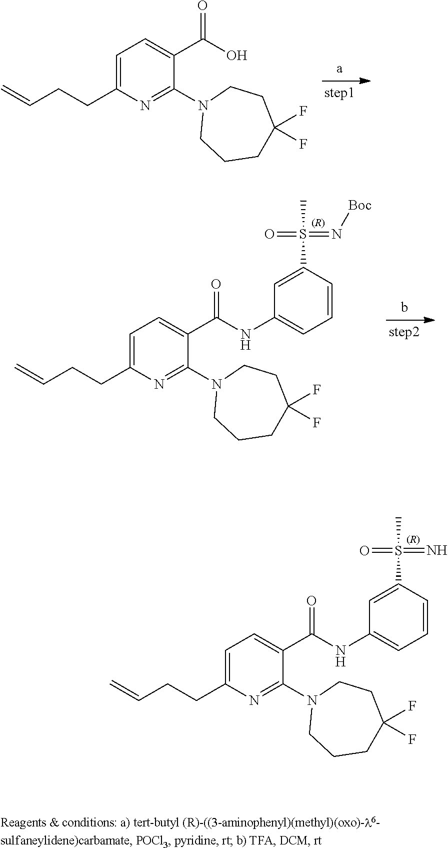

Step 1: tert-butyl (R)-((3-(6-(but-3-en-1-yl)-2-(4,4-difluoroazepan-1-yl)nicotinamido)phenyl)(methyl)(oxo)-λ⁶-sulfaneylidene)carbamate: A mixture of 6-(but-3-en-1-yl)-2-(4,4-difluoroazepan-1-yl)nicotinic acid (250 mg, 0.806 mmol) and tert-butyl (R)-((3-aminophenyl)(methyl)(oxo)-λ⁶-sulfaneylidene)carbamate (239 mg, 0.887 mmol) in pyridine (4 mL) was added POCl₃ (350 L) at 50° C. The mixture was stirred at room temperature for 1 h, then cooled to room temperature. The resulting solution was diluted with water (15 mL) and extracted with DCM (15 mL×3). The combined organic phases were washed with brine, dried over Na₂SO₄, concentrated under vacuum. The residue was purified by flash column chromatography on silica gel (PE/EtAOc=1/1) to give tert-butyl (R)-((3-(6-(but-3-en-1-yl)-2-(4,4-difluoroazepan-1-yl)nicotinamido)phenyl)(methyl)(oxo)-λ⁶-sulfaneylidene)carbamate (60 mg, 13%) as a yellow oil. LCMS (ESI) calcd. for C₂₈H₃₇F₂N₄O₄S [M+H]⁺ m/z 563.25, found 563.15.

Step 2: (R)-6-(but-3-en-1-yl)-2-(4,4-difluoroazepan-1-yl)-N-(3-(S-methylsulfonimidoyl)phenyl)nicotinamide: A solution of tert-butyl (R)-((3-(6-(but-3-en-1-yl)-2-(4,4-difluoroazepan-1-yl)nicotinamido)phenyl)(methyl)(oxo)-λ⁶-sulfaneylidene)carbamate (80 mg, 0.143 mmol) in DCM (3 mL) was added TFA (1 mL) at room temperature. The reaction mixture was stirred at room temperature for 2 hours. After the reaction was completed, the solution was diluted with water (10 mL) and extracted with DCM (10 mL×3). The combined organic phases were washed with brine, dried over sodium sulfate, and concentrated under vacuum. The residue was purified by prep-HPLC (Gemini 5 um C₁₈ column, 150×21.2 mm, eluting with 50% to 80% ACN-H₂O containing 0.1% formic acid) to obtain (R)-6-(but-3-en-1-yl)-2-(4,4-difluoroazepan-1-yl)-N-(3-(S-methylsulfonimidoyl)phenyl)nicotinamide (23 mg, 35%) as a white solid. ¹H NMR (400 MHz, DMSO-d₆) δ 10.73 (s, 1H), 8.43 (s, 1H), 7.68-7.63 (m, 1H), 7.62-7.61 (m, 3H), 6.66 (d, J=7.6 Hz, 1H), 5.89-5.82 (m, 1H), 5.02-4.95 (m, 2H), 3.64-3.62 (m, 2H), 3.43-3.40 (m, 2H), 3.31 (s, 3H), 2.75-2.71 (m, 2H), 2.45-2.43 (m, 2H), 2.42-2.32 (m, 2H), 1.93-1.84 (m, 4H). LCMS (ESI) calcd. for C₂₃H₂₉F₂N₄O₂S [M+H]⁺ m/z 463.20, found 463.05.

### Example 36

### 4-(Azepan-1-yl)-N-(2-oxo-1,2-dihydropyridin-4-yl)-6-(trifluoromethyl)pyridazine-3-carboxamide

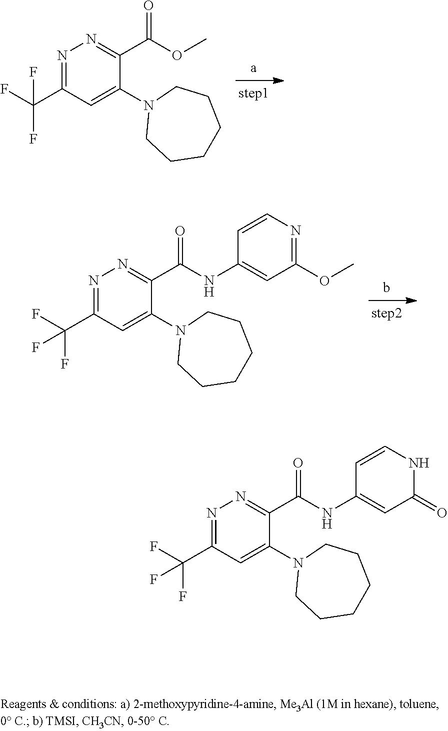 [SMILES: COC(=O)c1nnc(C(F)(F)F)cc1N1CCCCCC1 ; COc1cc(NC(=O)c2nnc(C(F)(F)F)cc2N2CCCCCC2)ccn1 ; O=C(Nc1cc[nH]c(=O)c1)c1nnc(C(F)(F)F)cc1N1CCCCCC1]

Step 1: 4-(azepan-1-yl)-N-(2-methoxypyridin-4-yl)-6-(trifluoromethyl) pyridazine-3-carboxamide: To a solution of 2-methoxypyridin-4-amine (368 mg, 2.97 mmol) in toluene (4 mL) was added Me₃Al (1M in hexane, 3.26 mL, 3.26 mmol) dropwise at 0° C. The mixture was stirred at 0° C. for 0.5 h. Then a solution of methyl 4-(azepan-1-yl)-6-(trifluoromethyl)pyridazine-3-carboxylate (300 mg, 0.99 mmol) in toluene (2 mL) was added at the same temperature. The mixture was heated at 90° C. for 2 hours. LCMS showed the reaction was completed. The mixture was diluted with water (30 mL) and extracted with EtOAc (20 mL×3). The combined organic phases were washed with brine, dried with Na₂SO₄, concentrated, and purified by silica gel column chromatography (eluting with EtOAc/PE=1/1) to give 4-(azepan-1-yl)-N-(2-methoxypyridin-4-yl)-6-(trifluoromethyl) pyridazine-3-carboxamide (300 mg, 76.9%) as a yellow solid. LCMS (ESI) calcd. for C₁₈H₂₁F₃N₅O₂ [M+H]⁺ m/z 396.16, found 396.00.

Step 2: 4-(azepan-1-yl)-N-(2-oxo-1,2-dihydropyridin-4-yl)-6-(trifluoromethyl)pyridazine-3-carboxamide: To a solution of 4-(azepan-1-yl)-N-(2-methoxypyridin-4-yl)-6-(trifluoromethyl)pyridazine-3-carboxamide 7 (200 mg, 0.51 mmol) in ACN (3 mL) was added TMSI (141 mg, 1.01 mmol) at room temperature. The mixture was heated at 50° C. for 5 hours. LCMS showed the reaction was completed. The final mixture was quenched with water and extracted with EtOAc. The combined organic phases were washed with water and brine, dried with sodium sulfate, concentrated, and purified by Prep-HPLC (Column: Gemini-C18, 150×21.2 mm, 5 um; Mobile Phase: ACN-H₂O (0.1% FA), Gradient: 20%-38%-60%) to give 4-(azepan-1-yl)-N-(2-oxo-1,2-dihydropyridin-4-yl)-6-(trifluoromethyl)pyridazine-3-carboxamide (15 mg, 7.8%) as a white solid. ¹H NMR (400 MHz, DMSO-d₆) δ 11.31 (s, 1H), 11.05 (s, 1H), 7.35 (t, J=3.5 Hz, 2H), 6.84 (d, J=1.9 Hz, 1H), 6.44 (dd, J=7.2, 1.8 Hz, 1H), 3.57-3.50 (m, 4H), 1.74 (s, 4H), 1.48 (s, 4H). LCMS (ESI) calcd. for C₁₇H₁₉F₃N₅O₂ [M+H]⁺ m/z 382.15, found 382.00.

### Example 37

### 4-(Azepan-1-yl)-N-(3-(methylsulfonyl)phenyl)-6-(trifluoromethyl)pyridazine-3-carboxamide

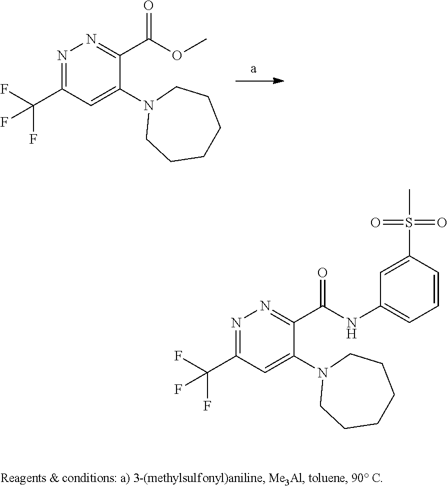 [SMILES: COC(=O)c1nnc(C(F)(F)F)cc1N1CCCCCC1 ; CS(=O)(=O)c1cccc(NC(=O)c2nnc(C(F)(F)F)cc2N2CCCCCC2)c1]

To a solution of 3-(methylsulfonyl)aniline (338 mg, 1.98 mmol) in toluene (5 mL) was added Me₃Al (1M in hexane, 2.18 mL, 2.18 mmol) dropwise at 0° C. The mixture was stirred at 0° C. for 0.5 h. Then a solution of methyl 4-(azepan-1-yl)-6-(trifluoromethyl)pyridazine-3-carboxylate (200 mg, 0.66 mmol) in toluene (3 mL) was added at the same temperature. The resulting mixture was heated at 90° C. for 2 hours. LCMS showed the reaction was completed. The mixture was quenched with water (30 mL) and extracted with EtOAc (20 mL×3). The combined organic phases were washed with brine, dried with Na₂SO₄, concentrated, purified by prep-HPLC (Column: Gemini-C₁₈, 150×21.2 mm, 5 um; Mobile Phase: ACN-H₂O (0.1% FA), Gradient: 20%-38%-60%) to give 4-(azepan-1-yl)-N-(3-(methylsulfonyl)phenyl)-6-(trifluoromethyl)pyridazine-3-carboxamide (80 mg, 28%) as a white solid. ¹H NMR (400 MHz, DMSO-d₆) δ 11.41 (s, 1H), 8.39 (s, 1H), 8.01 (d, J=7.5 Hz, 1H), 7.76-7.62 (m, 2H), 7.36 (s, 1H), 3.63-3.52 (m, 4H), 3.24 (s, 3H), 1.74 (s, 4H), 1.47 (s, 4H). LCMS (ESI) calcd. for C₁₉H₂₂F₃N₄O₃S [M+H]⁺ m/z 443.13, found 443.10.

### Example 38

### N-(3-Carbamoylphenyl)-4-(4,4-difluoroazepan-1-yl)-6-(trifluoromethyl)pyridazine-3-carboxamide

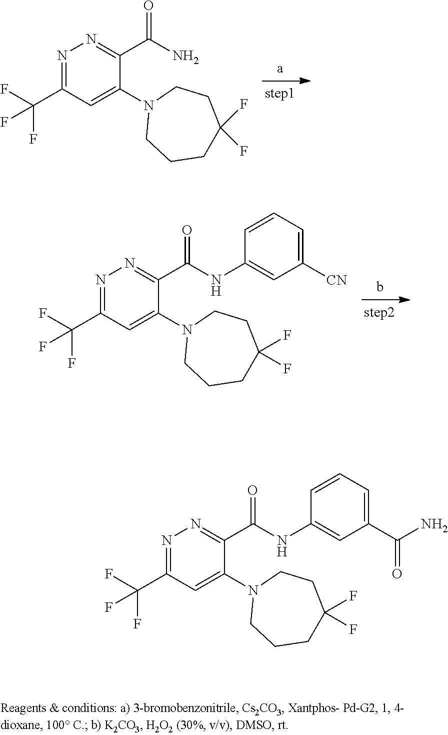 [SMILES: NC(=O)c1nnc(C(F)(F)F)cc1N1CCCC(F)(F)CC1 ; N#Cc1cccc(NC(=O)c2nnc(C(F)(F)F)cc2N2CCCC(F)(F)CC2)c1 ; NC(=O)c1cccc(NC(=O)c2nnc(C(F)(F)F)cc2N2CCCC(F)(F)CC2)c1]

Step 1: A mixture of 4-(4,4-difluoroazepan-1-yl)-6-(trifluoromethyl)pyridazine-3-carboxamide (250 mg, 0.62 mmol), 3-bromobenzonitrile (335 mg, 1.86 mmol), Cs₂CO₃(606 mg, 1.86 mmol) and Xantphos-Pd-G2 (52 mg, 0.06 mmol) in 1,4-dioxane (5 mL) was heated at 100° C. overnight under an atmosphere of N₂. LCMS showed the reaction was completed. The mixture was filtered through celite and the filtrate was concentrated under vacuum. The residue was purified by flash column chromatography on silica gel (PE/EtOAc=4/1) to give N-(3-cyanophenyl)-4-(4,4-difluoroazepan-1-yl)-6-(trifluoromethyl)pyridazine-3-carboxamide (200 mg, 75.7%) as a white solid. LCMS (ESI) calcd. for C₁₉H₁₇F₅N₅O [M+H]⁺ m/z 426.14, found 426.00.

Step 2: N-(3-carbamoylphenyl)-4-(4,4-difluoroazepan-1-yl)-6-(trifluoromethyl)pyridazine-3-carboxamide: To a solution of N-(3-cyanophenyl)-4-(4,4-difluoroazepan-1-yl)-6-(trifluoromethyl)pyridazine-3-carboxamide (100 mg, 0.24 mmol) in DMSO (5 mL) was added K₂CO₃ (100 mg, 0.72 mmol) and H₂O₂(30% solution, 0.5 mL). The solution was stirred at room temperature for 4 hours. LCMS showed the reaction was completed. The resulting mixture was diluted with water and extracted with EtOAc. The combined organic phases were washed with water and brine, dried over sodium sulfate, and concentrated under vacuum. The residue was purified by prep-HPLC (Column: Gemini-C18, 150×21.2 mm, 5 um; Mobile Phase: ACN-H₂O (0.05% NH₃), Gradient: 20%-80%) to give N-(3-carbamoylphenyl)-4-(4,4-difluoroazepan-1-yl)-6-(trifluoromethyl)pyridazine-3-carboxamide (40 mg, 38%) as a white solid. LCMS (ESI) calcd. for C₁₉H₂₀F₅N₅O₂ [M+H]⁺ m/z 444.15, found 444.30. ¹H NMR (400 MHz, DMSO-d₆) δ 11.11 (s, 1H), 8.22 (s, 1H), 7.99 (s, 1H), 7.86 (d, J=8.2 Hz, 1H), 7.64 (d, J=7.6 Hz, 1H), 7.46 (t, J=7.9 Hz, 1H), 7.41 (s, 2H), 3.72-3.63 (m, 2H), 3.58 (t, J=5.7 Hz, 2H), 2.37-2.24 (m, 2H), 2.13-2.19 (m, 2H), 1.93-1.84 (m, 2H).

### Example 39

### 4-(4,4-Difluoroazepan-1-yl)-N-(3-(methylsulfonyl)phenyl)-6-(trifluoromethyl)pyridazine-3-carboxamide

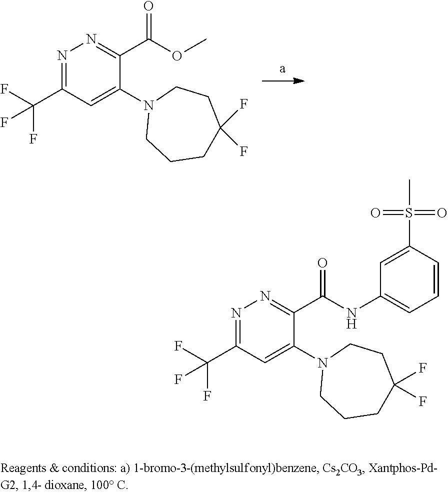 [SMILES: COC(=O)c1nnc(C(F)(F)F)cc1N1CCCC(F)(F)CC1 ; CS(=O)(=O)c1cccc(NC(=O)c2nnc(C(F)(F)F)cc2N2CCCC(F)(F)CC2)c1]

A solution of 4-(4,4-difluoroazepan-1-yl)-6-(trifluoromethyl)pyridazine-3-carboxamide (100 mg, 0.30 mmol), 1-bromo-3-(methylsulfonyl)benzene (143 mg, 0.61 mmol), Cs₂CO₃ (293 mg, 0.9 mmol) and Xantphos-Pd-G2 (27 mg, 0.03 mmol) in 1,4-dioxane (3 mL) was heated at 100° C. overnight under an atmosphere of N₂. LCMS showed the reaction was complete. The mixture was filtered through celite and the filtrate was concentrated under vacuum. The residue was purified by prep-HPLC (Column: Gemini-C18, 150×21.2 mm, 5 um; Mobile Phase: ACN-H₂O (0.05% FA), Gradient: 35%-85%) to give 4-(4,4-difluoroazepan-1-yl)-N-(3-(methylsulfonyl)phenyl)-6-(trifluoromethyl)pyridazine-3-carboxamide (36 mg, 25%) as a white solid. ¹H NMR (400 MHz, DMSO-d₆) δ 11.41 (s, 1H), 8.40 (s, 1H), 8.05 (d, J=7.5 Hz, 1H), 7.88-7.61 (m, 2H), 7.44 (s, 1H), 3.70-3.63 (m, 2H), 3.55 (t, J=5.7 Hz, 2H), 3.24 (s, 3H), 2.37-2.24 (s, 2H), 2.16-1.96 (m, 2H), 1.94-1.83 (m, 2H). LCMS (ESI) calcd. for C₁₉H₂₀F₅N₄O₃S [M+H]⁺ m/z 479.12, found 479.05.

### Example 40

### 4-(4,4-Difluoroazepan-1-yl)-N-(2-oxo-1,2-dihydropyridin-4-yl)-6-(trifluoromethyl)pyridazine-3-carboxamide

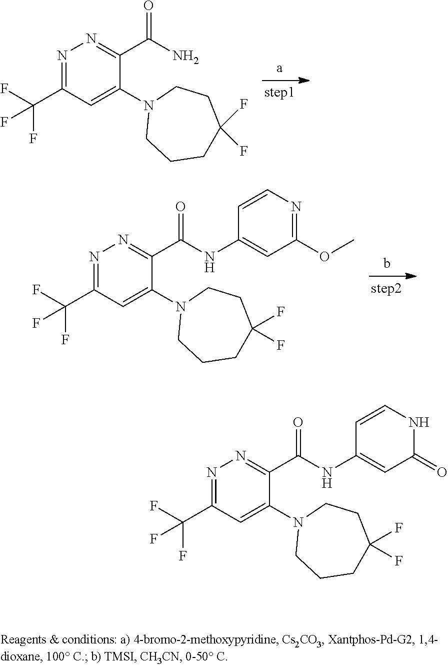

Step 1: 4-(4,4-difluoroazepan-1-yl)-N-(2-methoxypyridin-4-yl)-6-(trifluoromethyl)pyridazine-3-carboxamide: A mixture of 4-(4,4-difluoroazepan-1-yl)-6-(trifluoromethyl)pyridazine-3-carboxamide (250 mg, 0.77 mmol), 4-bromo-2-methoxypyridine (215 mg, 1.16 mmol), Cs₂CO₃ (625 mg, 1.93 mmol) and Xantphos-Pd-G2 (18 mg, 0.02 mmol) in dioxane (5 mL) was heated at 100° C. for 16 hours. LCMS showed the reaction was completed. The mixture was diluted with water (20 mL) and extracted with EtOAc (20 mL×3). The combined organic phases were washed with brine, dried over sodium sulfate, concentrated under vacuum. The residue was purified by flash column chromatography on silica gel (PE/EtOAc=1/3) to provide 4-(4,4-difluoroazepan-1-yl)-N-(2-methoxypyridin-4-yl)-6-(trifluoromethyl)pyridazine-3-carboxamide (200 mg, 61.9%) as a yellow solid. LCMS (ESI) calcd. for C₁₈H₁₉F₅N₅O₂ [M+H]⁺ m/z 432.15, found 432.10.

Step 2: 4-(4,4-difluoroazepan-1-yl)-N-(2-oxo-1,2-dihydropyridin-4-yl)-6-(trifluoromethyl)pyridazine-3-carboxamide: To a solution of 4-(4,4-difluoroazepan-1-yl)-N-(2-methoxypyridin-4-yl)-6-(trifluoromethyl) pyridazine-3-carboxamide (150 mg, 0.35 mmol) in MeCN (2 mL) was added TMSI (97 mg, 0.70 mmol) at room temperature. The mixture was heated at 50° C. for 5 hours. LCMS showed the reaction was completed. The final mixture was concentrated under vacuum. The residue was first purified by flash column chromatography on silica gel (DCM/MeOH=10/1), then further purified by prep-HPLC (Gemini 5 um C₁₈ column, 150×21.2 mm, eluting with 30% to 90% ACN-H₂O containing 0.1% formic acid) to afford 4-(4,4-difluoroazepan-1-yl)-N-(2-oxo-1,2-dihydropyridin-4-yl)-6-(trifluoromethyl)pyridazine-3-carboxamide (62 mg, 43%) as a white solid. ¹H NMR (400 MHz, DMSO-d₆) δ 11.34 (s, 1H), 11.08 (s, 1H), 7.44 (s, 1H), 7.36 (d, J=7.0 Hz, 1H), 6.86 (d, J=1.8 Hz, 1H), 6.47 (d, J=7.1 Hz, 1H), 3.70-3.58 (m, 2H), 3.51 (t, J=5.6 Hz, 2H), 2.36-2.24 (m, 2H), 2.11-2.01 (m, 2H), 1.92-1.85 (m, 2H). LCMS (ESI) calcd. for C₁₇H₁₇F₅N₅O₂ [M+H]⁺ m/z 418.13, found 418.05.

### Example 41

### 5-Chloro-2-(4,4-difluoroazepan-1-yl)-4-methyl-N-(3-(S-methylsulfonimidoyl)phenyl)-6-(trifluoromethyl)nicotinamide

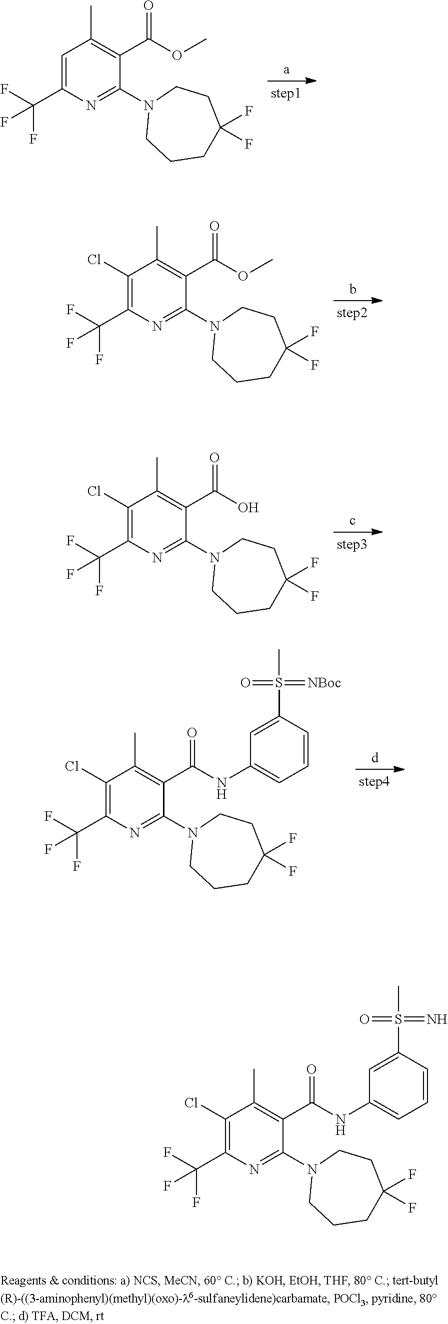

Step 1: methyl 5-chloro-2-(4,4-difluoroazepan-1-yl)-4-methyl-6-(trifluoromethyl)nicotinate: To a solution of methyl 2-(4,4-difluoroazepan-1-yl)-4-methyl-6-(trifluoromethyl)nicotinate (500 mg, 1.42 mmol) in MeCN (10 mL) was added NCS (228 mg, 1.70 mmol) at 25° C. The mixture was heated at 60° C. for 6 hours. The mixture was quenched with water (50 mL) and extracted with EtOAc (50 mL×2). The combine organic phases were washed with brine, dried over sodium sulfate, concentrated under vacuum. The residue was purified by flash column chromatography on silica gel (PE/EtOAc=3/1) to provide methyl 5-chloro-2-(4,4-difluoroazepan-1-yl)-4-methyl-6-(trifluoromethyl)nicotinate (450 mg, 79.0%) as a yellow solid. LCMS (ESI) calcd. for C₁₆H₁₉ClF₅N₂O₂ [M+H]⁺ m/z 401.11, found 400.95.

Step 2: 5-chloro-2-(4,4-difluoroazepan-1-yl)-4-methyl-6-(trifluoromethyl)nicotinic acid: To a solution of ethyl 5-chloro-2-(4,4-difluoroazepan-1-yl)-4-methyl-6-(trifluoromethyl)nicotinate (450 mg, 1.12 mmol) in THF/EtOH/H₂O (1/1, 10 mL) was added KOH (1.25 g, 22.44 mmol) at room temperature. The mixture was heated at 80° C. for 5 hours. After the reaction was completed, the mixture was concentrated to remove most THE and EtOH. The aqueous phase was adjusted to pH=3-4 with 1N HCl then extracted with EtOAc (20 mL×3). The combined organic phases were washed with brine, dried with Na₂SO₄, and concentrated under reduced pressure to afford 5-chloro-2-(4,4-difluoroazepan-1-yl)-4-methyl-6-(trifluoromethyl)nicotinic acid (350 mg, 83.7%) as a yellow solid. LCMS (ESI) calcd. for C₁₄H₁₅ClF₅N₂O₂ [M+H]⁺ m/z 373.08, found 373.05.

Step 3: tert-butyl ((3-(5-chloro-2-(4,4-difluoroazepan-1-yl)-4-methyl-6-(trifluoromethyl)nicotinamido)phenyl)(methyl)(oxo)-16-sulfaneylidene)carbamate: A mixture of 5-chloro-2-(4,4-difluoroazepan-1-yl)-4-methyl-6-(trifluoromethyl)nicotinic acid (200 mg, 0.73 mmol) and tert-butyl ((3-aminophenyl)(methyl)(oxo)-16-sulfaneylidene)carbamate (174 mg, 0.87 mmol) in pyridine (5 mL) was added POCl₃ (150 L) dropwise at room temperature. The reaction solution was stirred at room temperature for 1 hour. After the reaction was completed, the resulting solution was diluted with water (20 mL) and extracted with DCM (20 mL×3). The combined organic phases were washed with brine, dried over sodium sulfate, concentrated under vacuum. The residue was purified by flash column chromatography on silica gel (PE/EtOAc=2/1) to give tert-butyl ((3-(5-chloro-2-(4,4-difluoroazepan-1-yl)-4-methyl-6-(trifluoromethyl)nicotinamido)phenyl)(methyl)(oxo)-16-sulfaneylidene)carbamate (50 mg, 28.6%) as a yellow solid. LCMS (ESI) calcd. for C₂₆H₃₀ClF₅N₄O₄SNa [M+H]⁺ m/z 647.15, found 647.10.

Step 4: 5-chloro-2-(4,4-difluoroazepan-1-yl)-4-methyl-N-(3-(S-methylsulfonimidoyl)phenyl)-6-(trifluoromethyl)nicotinamide: A solution of tert-butyl ((3-(5-chloro-2-(4,4-difluoroazepan-1-yl)-4-methyl-6-(trifluoromethyl)nicotinamido)phenyl)(methyl)(oxo)-λ⁶-sulfaneylidene)carbamate (50 mg, 0.08 mmol) in DCM (3 mL) was added TFA (1 mL) at room temperature. The reaction mixture was stirred at room temperature for 2 hours. After the reaction was completed, the mixture was diluted with water (10 mL) and extracted with DCM (10 mL×3). The combined organic phases were washed with brine, dried over sodium sulfate, concentrated under vacuum. The residue was purified by prep-HPLC (Gemini 5 um C₁₈ column, 150*21.2 mm, eluting with 30% to 85% MeCN/H₂O containing 0.1% formic acid) to provide 5-chloro-2-(4,4-difluoroazepan-1-yl)-4-methyl-N-(3-(S-methylsulfonimidoyl)phenyl)-6-(trifluoromethyl)nicotinamide (14.1 mg, 33.5%) as a white solid. ¹H NMR (400 MHz, DMSO-d₆) δ 10.98 (s, 1H), 8.29 (s, 1H), 7.89 (d, J=8.2 Hz, 1H), 7.70 (d, J=7.9 Hz, 1H), 7.61 (t, J=7.9 Hz, 1H), 4.24 (s, 1H), 3.62 (dt, J=12.0, 5.3 Hz, 4H), 3.07 (s, 3H), 2.41-2.21 (m, 5H), 2.08-1.96 (m, 2H), 1.86-1.78 (m, 2H). LCMS (ESI) calcd. for C₂₁H₂₃ClF₅N₄O₂S [M+H]⁺ m/z 525.12, found 525.10.

### Example 42

### 2-(4,4-Difluoroazepan-1-yl)-4-methyl-N-(3-(S-methylsulfonimidoyl)phenyl)-5-phenylnicotinamide

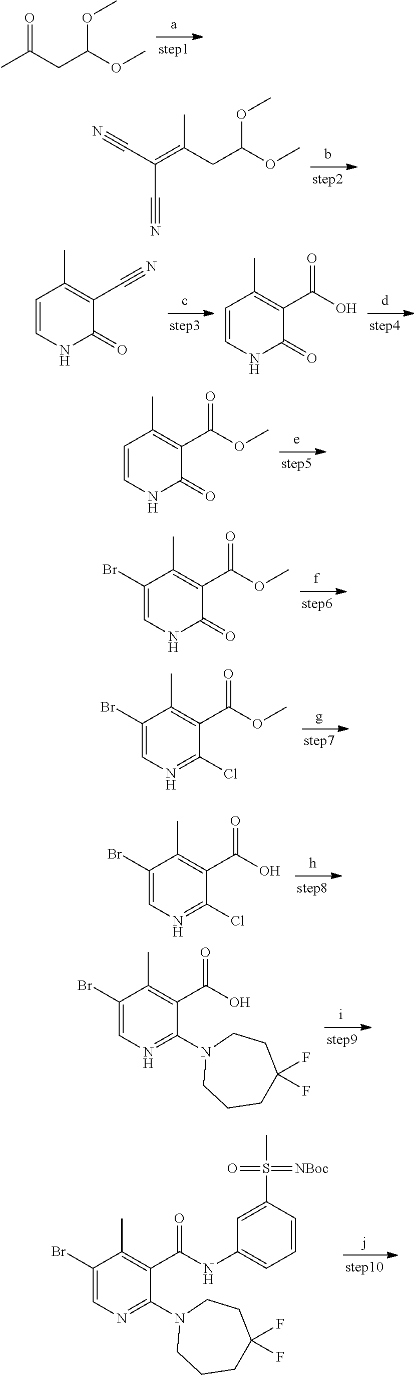

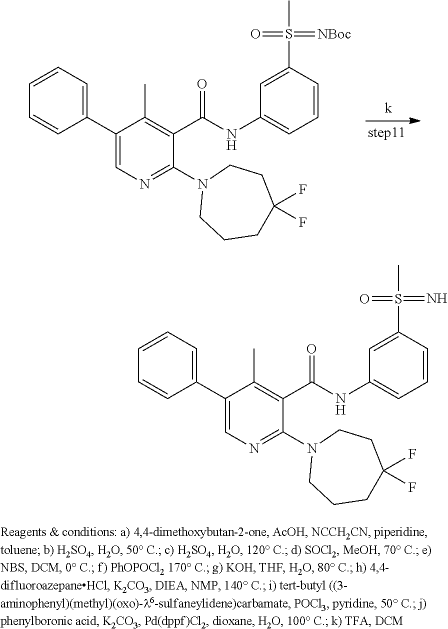

Step 1: 2-(4,4-dimethoxybutan-2-ylidene)malononitrile: To a stirred solution of 4,4-dimethoxybutan-2-one (100 g, 0.76 mol), acetic acid ((4.54 g, 0.076 mol) and malononitrile (45 g, 0.68 mol) in toluene (250 mL) was added piperidine (6.44 g, 0.076 mol) in portions over 20 minutes. The reaction mixture was stirred overnight at room temperature. The mixture was washed with H₂O (200 mL), dried over Na₂SO₄ and concentrated in vacuo to afford the crude 2-(4,4-dimethoxybutan-2-ylidene)malononitrile (50 g) which was used directly in the next step.

Step 2: 4-methyl-2-oxo-1,2-dihydropyridine-3-carbonitrile: Crude 2-(4,4-dimethoxybutan-2-ylidene)malononitrile (50 g, about 278 mmol) was added dropwise to a stirred solution of concentrated H₂SO₄ (30 mL) at a rate that the reaction temperature did not exceed 30° C. The reaction mixture was then heated at 50° C. for 2 h. The resulting reaction mixture was cooled to room temperature and added slowly into ice-H₂O (200 mL). The precipitate was collected by filtration, washed with water, and dried under vacuum to give the 4-methyl-2-oxo-1,2-dihydropyridine-3-carbonitrile (25 g, 67%) as white solid. LCMS (ESI) calcd. for C₇H₇N₂O [M+H]⁺ m/z 135.06, found 134.95.

Step 3: 4-methyl-2-oxo-1,2-dihydropyridine-3-carboxylic acid: A solution of 4-methyl-2-oxo-1,2-dihydropyridine-3-carbonitrile (25 g, 187 mmol) in 50% aqueous H₂SO₄ (40 mL) was heated at 120° C. for 8 hours. Then the reaction mixture was cooled to room temperature slowly poured into ice-water. The precipitate was collected by filtration, washed with water, and dried under vacuum to give crude 4-methyl-2-oxo-1,2-dihydropyridine-3-carboxylic acid (15 g, 52.1% yield) which was used in the next step without further purification. LCMS (ESI) calcd. for C₇H₇NO₃ [M+H]⁺ m/z 154.05, found 154.0.

Step 4: methyl 4-methyl-2-oxo-1,2-dihydropyridine-3-carboxylate: To a solution of 4-methyl-2-oxo-1,2-dihydropyridine-3-carboxylic acid (10 g, 65.3 mmol) in MeOH (120 mL) was added SOC₂ (15 mL) dropwise. Then the mixture was heated at 65° C. for 16 hours. LCMS showed the reaction was completed. The mixture was concentrated to give the crude. The crude was diluted with ice-water, extracted with DCM (3×80 mL). The combined organic phases were washed with brine, dried with Na₂SO₄, and concentrated under reduced pressure to afford crude methyl 4-methyl-2-oxo-1,2-dihydropyridine-3-carboxylate (10 g, 91.7%) which was used in the next step without further purification. LCMS (ESI) calcd. for C₅H₁₀NO₃ [M+H]⁺ m/z 168.07, found 168.0.

Step 5: methyl 5-bromo-4-methyl-2-oxo-1,2-dihydropyridine-3-carboxylate: To a solution of methyl 4-methyl-2-oxo-1,2-dihydropyridine-3-carboxylate (10.0 g, 59.8 mmol) in DCM (100 mL) was added NBS (10.64 g, 59.8 mmol) at 0° C. The solution was stirred at the same temperature for 30 minutes. Then the mixture was washed with brine, dried with Na₂SO₄, and concentrated under reduced pressure to afford crude methyl 5-bromo-4-methyl-2-oxo-1,2-dihydropyridine-3-carboxylate (16.5 g, 80% purity, 89.6%). LCMS (ESI) calcd. for C₈H₈BrNO₃ [M+H]⁺ m/z 245.98, found 246.

Step 6: methyl 5-bromo-2-chloro-4-methylnicotinate: A solution of methyl 5-bromo-4-methyl-2-oxo-1,2-dihydropyridine-3-carboxylate (4.0 g, 16.3 mmol) in phenyl dichlorophosphate (30 mL) was heated at 170° C. for 4 hours. The solution was cooled to room temperature, diluted with water, and extracted with DCM (2×70 mL). The combined organics were washed with brine (100 mL), dried over Na₂SO₄, concentrated under reduced pressure to afford the crude. The crude was purified by flash column chromatography on silica gel (PE/EtOAc=12/1) to give methyl 5-bromo-2-chloro-4-methylnicotinate (2.7 g, 63.2%). LCMS (ESI) calcd. for C₈H₈BrClNO₂ [M+H]⁺ m/z 263.94, found 263.75.

Step 7: 5-bromo-2-chloro-4-methylnicotinic acid: To a solution of methyl 5-bromo-2-chloro-4-methylnicotinate (1.5 g, 5.7 mmol) in THF/H₂O (1/1, 30 mL) was added KOH (3.19 g, 57 mmol) at room temperature. The mixture was heated at 80° C. for 16 hours. After the reaction was completed, the mixture was concentrated to remove most THF. The aqueous phase was adjusted to pH=3-4 with 1N HCl then extracted with EtOAc (50 mL×3). The combined organic phases were washed with brine, dried with Na₂SO₄, and concentrated under reduced pressure to afford 5-bromo-2-chloro-4-methylnicotinic acid (1.30 g, 90.9%) as a white solid. LCMS (ESI) calcd. for C₇H₆BrClNO₂ [M+H]⁺ m/z 249.93, found 251.85.

Step 8: 5-bromo-2-(4,4-difluoroazepan-1-yl)-4-methylnicotinic acid: A solution of 5-bromo-2-chloro-4-methylnicotinic acid (400 mg, 1.59 mmol), 4,4-difluoroazepane hydrochloride (543 mg, 3.18 mmol), K₂CO₃ (1.43 g, 10.34 mmol) and DIEA (821 mg, 6.36 mmol) in NMP (10 mL) was heated at 140° C. for 6 hours. LCMS showed the reaction was completed. The mixture concentrated under vacuum and directly purified by flash column chromatography on silica gel (PE/EtOAc=2/1 to 1/1) to provide 5-bromo-2-(4,4-difluoroazepan-1-yl)-4-methylnicotinic acid (320 mg, 60% purity, 34.7% yield) as a yellow solid. LCMS (ESI) calcd. for C₁₃H₁₆BrF₂N₂O₂ [M+H]⁺ m/z 351.04, found 350.9.

Step 9: tert-butyl ((3-(5-bromo-2-(4,4-difluoroazepan-1-yl)-4-methylnicotinamido)phenyl)(methyl)(oxo)-λ⁶-sulfaneylidene)carbamate: A mixture of 5-bromo-2-(4,4-difluoroazepan-1-yl)-4-methylnicotinic acid (150 mg, 0.43 mmol) and tert-butyl ((3-aminophenyl)(methyl)(oxo)-λ⁶-sulfaneylidene)carbamate (232 mg, 0.86 mmol) in pyridine (3 mL) was added POCl₃ (100 L) dropwise at 50° C. The reaction solution was stirred at 50° C. for 1 hour. After the reaction was completed, the resulting solution was diluted with water (20 mL) and extracted with DCM (20 mL×3). The combined organic phases were washed with brine, dried over sodium sulfate, concentrated under vacuum. The residue was purified by flash column chromatography on silica gel (PE/EtOAc=2/1) to give tert-butyl ((3-(5-bromo-2-(4,4-difluoroazepan-1-yl)-4-methylnicotinamido)phenyl)(methyl)(oxo)-λ⁶-sulfaneylidene)carbamate (80 mg, 31%) as a yellow solid. LCMS (ESI) calcd. for C₂₅H₃₂BrF₂N₄O₄S [M+H]⁺ m/z 603.13, found 603.05.

Step 10: tert-butyl ((3-(2-(4,4-difluoroazepan-1-yl)-4-methyl-5-phenylnicotinamido)phenyl)(methyl)(oxo)-λ⁶-sulfaneylidene)carbamate: A mixture of tert-butyl ((3-(5-bromo-2-(4,4-difluoroazepan-1-yl)-4-methylnicotinamido)phenyl)(methyl)(oxo)-λ⁶-sulfaneylidene)carbamate (50 mg, 0.083 mmol), phenylboronic acid (31 mg, 0.25 mmol), potassium carbonate (34.5 mg, 0.25 mmol) and [1,1′-Bis(diphenylphosphino)ferrocene]dichloropalladium (II) (6 mg, 0.008 mmol) in 1,4-dioxane/H₂O (4/1, 2 mL) was heated at 100° C. for 16 h under an atmosphere of N₂. After the reaction was completed, the mixture was cooled to room temperature. The resulting solution was diluted with water (20 mL) and extracted with EtOAc (20 mL×3). The combined organic phases were washed with brine, dried over Na₂SO₄, concentrated under vacuum. The residue was purified by flash column chromatography on silica gel (PE/EtAOc=3/1) to give tert-butyl ((3-(2-(4,4-difluoroazepan-1-yl)-4-methyl-5-phenylnicotinamido)phenyl)(methyl)(oxo)-λ⁶-sulfaneylidene)carbamate (40 mg, 80%) as a white oil. LCMS (ESI) calcd. for C₃₁H₃₇F₂N₄O₄S [M+H]⁺ m/z 599.25, found 599.20.

Step 11: 2-(4,4-difluoroazepan-1-yl)-4-methyl-N-(3-(S-methylsulfonimidoyl)phenyl)-5-phenylnicotinamide: A solution of tert-butyl ((3-(2-(4,4-difluoroazepan-1-yl)-4-methyl-5-phenylnicotinamido) phenyl)(methyl)(oxo)-λ⁶-sulfaneylidene)carbamate (40 mg, 0.067 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 30% to 85% ACN-H₂O containing 0.1% FA) to provide 2-(4,4-difluoroazepan-1-yl)-4-methyl-N-(3-(S-methylsulfonimidoyl)phenyl)-5-phenylnicotinamide (14.5 mg, 42.6%) as a white solid. ¹H NMR (400 MHz, DMSO-d₆) δ 10.88 (s, 1H), 8.38 (s, 1H), 8.08 (s, 1H), 7.90 (d, J=8.2 Hz, 1H), 7.66 (d, J=7.9 Hz, 1H), 7.58 (t, J=7.9 Hz, 1H), 7.47 (t, J=7.4 Hz, 2H), 7.37 (dd, J=19.8, 7.2 Hz, 3H), 4.22 (s, 1H), 3.58 (s, 4H), 3.06 (s, 3H), 2.31 (d, J=13.4 Hz, 2H), 2.15 (s, 3H), 2.01 (s, 2H), 1.82 (d, J=5.8 Hz, 2H). LCMS (ESI) calcd. for C₂₆H₂₉F₂N₄O₂S [M+H]⁺ m/z 499.20, found 499.10.

### Example 43

### (R)-3-(4,4-difluoroazepan-1-yl)-5-methyl-N-(3-(S-methyl-N-(methylglycyl)sulfonimidoyl)phenyl)-6-(trifluoromethyl)pyridazine-4-carboxamide

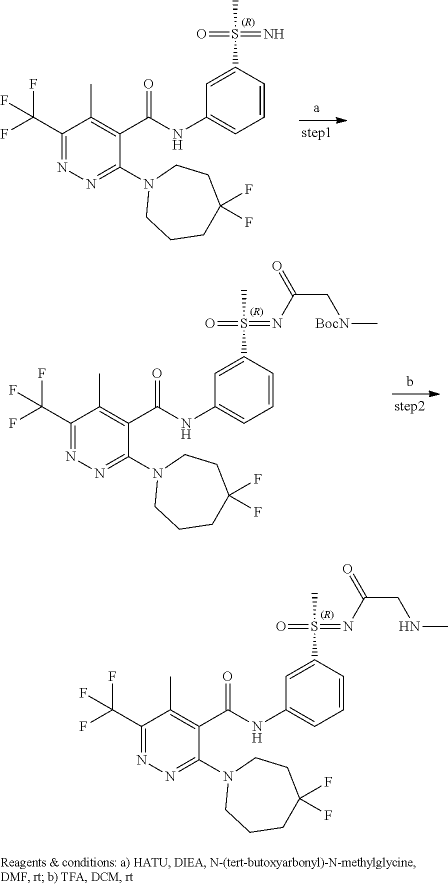

Step 1: tert-butyl (R)-(2-(((3-(3-(4,4-difluoroazepan-1-yl)-5-methyl-6-(trifluoromethyl)pyridazine-4-carboxamido)phenyl)(methyl)(oxo)-λ⁶-sulfaneylidene)amino)-2-oxoethyl)(methyl)carbamate: A mixture of (R)-3-(4,4-difluoroazepan-1-yl)-5-methyl-N-(3-(S-methylsulfonimidoyl)phenyl)-6-(trifluoromethyl)pyridazine-4-carboxamide (150 mg, 0.31 mmol) and N-(tert-butoxycarbonyl)-N-methylglycine (87 mg, 0.46 mmol) in DMF (10 mL) was added HATU (175 mg, 0.46 mmol) and DIEA (120 mg, 0.93 mmol). The mixture was stirred at room temperature for 4 hours. After the reaction was completed, the resulting solution was diluted with water (20 mL) and extracted with DCM (20 mL×3). The combined organic phases were washed with brine, dried over Na₂SO₄, concentrated under vacuum. The residue was purified by flash column chromatography on silica gel (PE/EtOAc=2/1 to 1/1) to give tert-butyl (R)-(2-(((3-(3-(4,4-difluoroazepan-1-yl)-5-methyl-6-(trifluoromethyl)pyridazine-4-carboxamido)phenyl)(methyl)(oxo)-λ⁶-sulfaneylidene)amino)-2-oxoethyl)(methyl)carbamate (110 mg, 53.6%) as a white oil. LCMS (ESI) calcd. for C₂₈H₃₆F₅N₆O₅S [M+H]⁺ m/z 663.24, found 663.20.

Step 2: (R)-3-(4,4-difluoroazepan-1-yl)-5-methyl-N-(3-(S-methyl-N-(methylglycyl)sulfonimidoyl)phenyl)-6-(trifluoromethyl)pyridazine-4-carboxamide: A solution of tert-butyl (R)-(2-(((3-(3-(4,4-difluoroazepan-1-yl)-5-methyl-6-(trifluoromethyl)pyridazine-4-carboxamido)phenyl)(methyl)(oxo)-λ⁶-sulfaneylidene)amino)-2-oxoethyl)(methyl)carbamate (110 mg, 0.16 mmol) in DCM (5 mL) was added TFA (0.5 mL) at room temperature. The reaction mixture was stirred at room temperature for 2 hours. After the reaction was completed, the solution was diluted with saturated aqueous NaHCO₃ (10 mL) and extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.1% NH₃) to obtain (R)-3-(4,4-difluoroazepan-1-yl)-5-methyl-N-(3-(S-methyl-N-(methylglycyl)sulfonimidoyl)phenyl)-6-(trifluoromethyl)pyridazine-4-carboxamide (51.3 mg, 56.9%) as a white solid. ¹H NMR (400 MHz, DMSO-d₆) δ 11.21 (s, 1H), 8.29 (s, 1H), 7.94 (d, J=7.8 Hz, 1H), 7.75-7.66 (m, 2H), 2.85 (s, 2H), 3.68 (t, J=6.0 Hz, 2H), 3.47 (s, 3H), 3.19 (s, 2H), 2.41-2.30 (m, 5H), 2.23 (s, 3 H), 2.14-1.97 (m, 2H), 1.95-1.83 (m, 2H). LCMS (ESI) calcd. for C₂₃H₂₈F₅N₆O₃S [M+H]⁺ m/z 563.19, found 563.

### Example 44

### (S)-3-(4,4-difluoroazepan-1-yl)-5-methyl-N-(3-(S-methyl-N-(methylglycyl)sulfonimidoyl)phenyl)-6-(trifluoromethyl)pyridazine-4-carboxamide

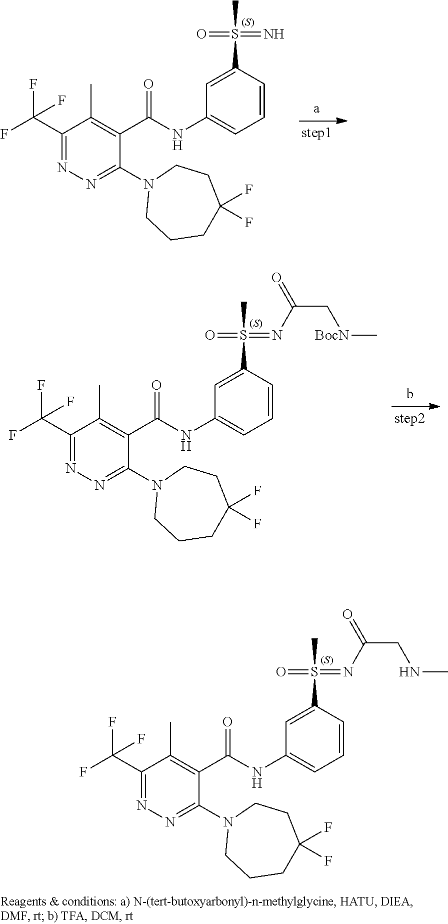

Step 1: tert-butyl (S)-(2-(((3-(3-(4,4-difluoroazepan-1-yl)-5-methyl-6-(trifluoromethyl)pyridazine-4-carboxamido)phenyl)(methyl)(oxo)-λ⁶-sulfaneylidene)amino)-2-oxoethyl)(methyl)carbamate: A mixture of (S)-3-(4,4-difluoroazepan-1-yl)-5-methyl-N-(3-(S-methylsulfonimidoyl)phenyl)-6-(trifluoromethyl)pyridazine-4-carboxamide(100 mg, 0.204 mmol) and N-(tert-butoxycarbonyl)-N-methylglycine (66 mg, 0.346 mmol) in DMF (10 mL) was added HATU (101 mg, 0.265 mmol) and DIEA (85 mg, 0.653 mmol). The mixture was stirred at room temperature for 4 hours. The reaction was monitored by LCMS. After the reaction was completed, the resulting solution was diluted with water (20 mL) and extracted with DCM (20 mL×3). The combined organic phases were washed with brine, dried over Na₂SO₄, concentrated under vacuum. The residue was purified by flash column chromatography on silica gel (PE/EtOAc=2/1 to 1/1) to give tert-butyl (S)-(2-(((3-(3-(4,4-difluoroazepan-1-yl)-5-methyl-6-(trifluoromethyl)pyridazine-4-carboxamido)phenyl)(methyl)(oxo)-λ⁶-sulfaneylidene)amino)-2-oxoethyl)(methyl)carbamate (85 mg, 62.96%) as a white oil. LCMS (ESI) calcd. for C₂₈H₃₆F₅N₆O₅S [M+H]⁺ m/z 663.24, found 663.15.

Step 2: (S)-3-(4,4-difluoroazepan-1-yl)-5-methyl-N-(3-(S-methyl-N-(methylglycyl)sulfonimidoyl)phenyl)-6-(trifluoromethyl)pyridazine-4-carboxamide: A solution of tert-butyl (S)-(2-(((3-(3-(4,4-difluoroazepan-1-yl)-5-methyl-6-(trifluoromethyl)pyridazine-4-carboxamido)phenyl)(methyl)(oxo)-λ⁶-sulfaneylidene)amino)-2-oxoethyl)(methyl)carbamate (85 mg, 0.128 mmol) in DCM (5 mL) was added TFA (0.5 mL) at room temperature. The reaction mixture was stirred at room temperature for 2 hours. After the reaction was completed, the solution was diluted with water (10 mL) and extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.1% formic acid) to obtain (S)-3-(4,4-difluoroazepan-1-yl)-5-methyl-N-(3-(S-methyl-N-(methylglycyl)sulfonimidoyl)phenyl)-6-(trifluoromethyl)pyridazine-4-carboxamide (43.98 mg, 60.93%) as a white solid. ¹H NMR (400 MHz, DMSO-d₆) δ 11.39 (bs, 1H), 8.28 (s, 1H), 7.92 (d, J=7.2 Hz, 1H), 7.67-7.63 (m, 2H), 3.85-3.78 (m, 2H), 3.71-3.68 (m, 2H), 3.45 (s, 3H), 3.14 (s, 2H), 2.40-2.38 (m, 2H), 2.32 (s, 3H), 2.23 (s, 3H), 2.08-2.01 (m, 2H), 1.89-1.87 (m, 2H). LCMS (ESI) calcd. for C₂₃H₂₈F₅N₆O₃S [M+H]⁺ m/z 563.19, found 563.15.

### Example 45

### (R)-2-(4,4-difluoroazepan-1-yl)-4-methyl-5-(1-methyl-1H-pyrazol-4-yl)-N-(3-(S-methylsulfonimidoyl)phenyl)nicotinamide

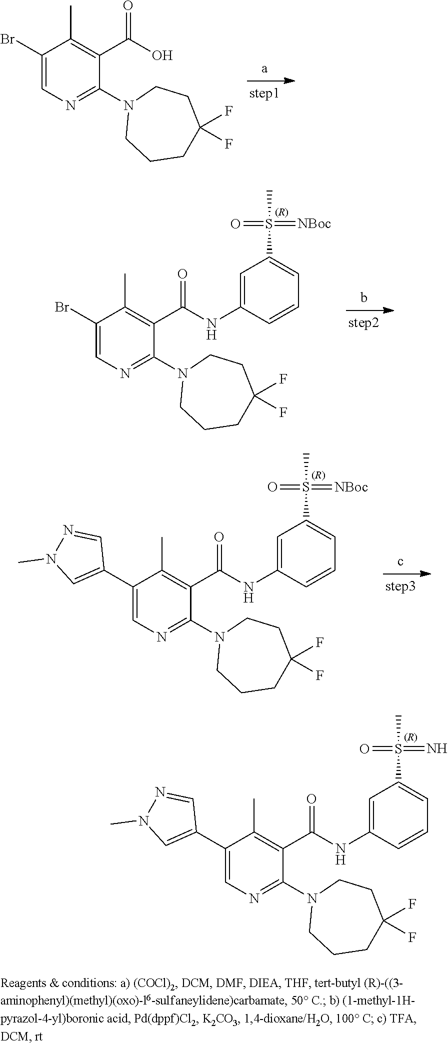

Step 1: tert-butyl (R)-((3-(5-bromo-2-(4,4-difluoroazepan-1-yl)-4-methylnicotinamido)phenyl)(methyl)(oxo)-λ⁶-sulfaneylidene)carbamate: To a solution of 5-bromo-2-(4,4-difluoroazepan-1-yl)-4-methylnicotinic acid (2 g, 5.75 mmol) in DCM (20 mL) was added (COCl)₂ (1.3 g, 10.34 mmol) and DMF (0.05 mL). The mixture was stirred at room temperature for 0.5 h. Then the mixture was concentrated in vacuum. The residue was diluted with THE (20 mL) and added to a solution of tert-butyl (R)-((3-aminophenyl)(methyl)(oxo)-16-sulfaneylidene)carbamate (1.7 g, 6.33 mmol) and DIEA (2.2 g, 17.25 mmol) in THE (20 mL). The reaction solution was heated at 50° C. for 1 hour. After the reaction was completed, the resulting solution was concentrated under vacuum. The residue was purified by flash column chromatography on silica gel (PE/EtOAc=1/2) to give tert-butyl (R)-((3-(5-bromo-2-(4,4-difluoroazepan-1-yl)-4-methylnicotinamido)phenyl)(methyl)(oxo)-λ⁶-sulfaneylidene)carbamate (2.1 g, 61.05%) as a yellow solid. LCMS (ESI) calcd. for C₂₅H₃₂BrF₂N₄O₄S [M+H]⁺ m/z 603.13, found 603.00.

Step 2: tert-butyl (R)-((3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)carbamate: A mixture of tert-butyl (R)-((3-(5-bromo-2-(4,4-difluoroazepan-1-yl)-4-methylnicotinamido)phenyl)(methyl)(oxo)-λ⁶-sulfaneylidene)carbamate (900 mg, 1.5 mmol), (1-methyl-1H-pyrazol-4-yl)boronic acid (378 mg, 3.0 mmol), K₂CO₃ (621 mg, 4.5 mmol) and [1,1′-Bis(diphenylphosphino)ferrocene]dichloropalladium (II) (120 mg, 0.16 mmol) in 1,4-dioxane/H₂O (4/1, 40 mL) was heated at 100° C. for 16 h under an atmosphere of N₂. After the reaction was completed, the mixture was diluted with water (100 mL) and extracted with EtOAc (80 mL×3). The combine organic phases were washed with brine, dried over Na₂SO₄, and concentrated under vacuum. The residue was purified by flash column chromatography on silica gel (PE/EtAOc=1/1) to provide tert-butyl (R)-((3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)carbamate (750 mg, 83%) as a white oil. LCMS (ESI) calcd. for C₂₉H₃₇F₂N₆O₄S [M+H]⁺ m/z 603.26, found 603.15.

Step 3: (R)-2-(4,4-difluoroazepan-1-yl)-4-methyl-5-(1-methyl-1H-pyrazol-4-yl)-N-(3-(S-methylsulfonimidoyl)phenyl)nicotinamide: A solution of tert-butyl (R)-((3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)carbamate (650 mg, 1.08 mmol) in DCM (10 mL) was added TFA (1 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (50 mL×3). The combined organic phases were washed with brine, dried over sodium sulfate, concentrated under vacuum. The residue was purified by prep-HPLC (Gemini 5 um C₁₈ column, 150*21.2 mm, eluting with 30% to 85% MeCN/H₂O containing 0.1% formic acid) to provide (R)-2-(4,4-difluoroazepan-1-yl)-4-methyl-5-(1-methyl-1H-pyrazol-4-yl)-N-(3-(S-methylsulfonimidoyl)phenyl)nicotinamide (311 mg, 57.4%) as a white solid. ¹H NMR (400 MHz, DMSO-d₆) δ 10.85 (s, 1H), 8.37 (s, 1H), 8.17 (s, 1H), 7.89 (d, J=3.4 Hz, 2H), 7.67 (d, J=7.8 Hz, 1H), 7.63-7.55 (m, 2H), 4.22 (s, 1H), 3.88 (s, 3H), 3.65-3.49 (m, 4H), 3.06 (s, 3H), 2.30-2.24 (m, 5H), 1.98 (t, J=11.3 Hz, 2H), 1.80 (d, J=5.6 Hz, 2H). LCMS (ESI) calcd. for C₂₄H₂₉F₂N₆O₂S [M+H]⁺ m/z 503.20, found 503.15.

### Example 46

### (R)-2-(4,4-difluoroazepan-1-yl)-4-methyl-5-(1-methyl-1H-pyrazol-4-yl)-N-(3-(S-methyl-N-(methylglycyl)sulfonimidoyl)phenyl)nicotinamide

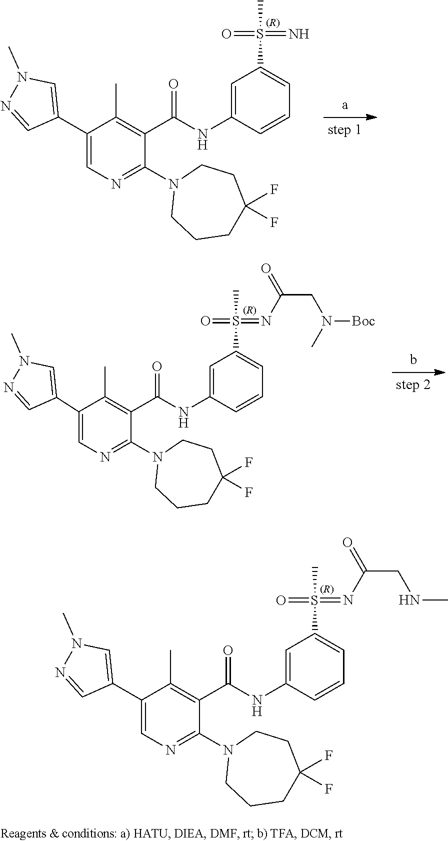

Step 1: tert-butyl (R)-(2-(((3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)amino)-2-oxoethyl)(methyl)carbamate: A solution of (R)-2-(4,4-difluoroazepan-1-yl)-4-methyl-5-(1-methyl-1H-pyrazol-4-yl)-N-(3-(S-methylsulfonimidoyl)phenyl)nicotinamide (60 mg, 0.12 mmol), N-(tert-butoxycarbonyl)-N-methylglycine (45.2 mg, 0.24 mmol) and HATU (59 mg, 0.155 mmol) in DMF (5 mL) was added DIEA (46.3 mg, 0.36 mmol) at room temperature. The reaction mixture was stirred at room temperature for 2 hours. After the reaction was completed, the mixture was diluted with water (20 mL) and extracted with DCM (20 mL×3). The combined organic phases were washed with brine, dried over sodium sulfate, concentrated under vacuum. The residue was purified by flash column chromatography on silica gel (PE/EtOAc=2/1) to give tert-butyl (R)-(2-(((3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)amino)-2-oxoethyl)(methyl)carbamate (55 mg, 68%) as a yellow oil. LCMS (ESI) calcd. for C₃₂H₄₂F₂N₇O₅S [M+H]⁺ m/z 674.29, found 674.25.

Step 2: (R)-2-(4,4-difluoroazepan-1-yl)-4-methyl-5-(1-methyl-1H-pyrazol-4-yl)-N-(3-(S-methyl-N-(methylglycyl)sulfonimidoyl)phenyl)nicotinamide: To a solution of tert-butyl (R)-(2-(((3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)amino)-2-oxoethyl)(methyl)carbamate (55 mg, 0.17 mmol) in DCM (5 mL) was added TFA (1 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 35% to 75% MeCN/H₂O containing 0.05% NH₃) to afford (R)-2-(4,4-difluoroazepan-1-yl)-4-methyl-5-(1-methyl-1H-pyrazol-4-yl)-N-(3-(S-methyl-N-(methylglycyl)sulfonimidoyl)phenyl)nicotinamide (15.3 mg, 16%) as a white solid. ¹H NMR (400 MHz, DMSO-d₆) δ 10.94 (s, 1H), 8.38 (s, 1H), 8.18 (s, 1H), 7.98 (dt, J=6.5, 2.1 Hz, 1H), 7.90 (s, 1H), 7.67 (dd, J=9.6, 4.2 Hz, 2H), 7.61 (s, 1H), 3.89 (s, 3H), 3.68-3.58 (m, 2H), 3.54 (t, J=6.1 Hz, 2H), 3.45 (s, 3H), 3.20 (s, 2H), 2.32-2.19 (m, 8H), 1.99 (dd, J=15.6, 9.9 Hz, 2H), 1.80 (d, J=5.7 Hz, 2H). LCMS (ESI) calcd. for C₂₇H₃₄F₂N₇O₃S [M+H]⁺ m/z 574.24, found 574.20.

### Example 47

### (R)-2-(4,4-difluoroazepan-1-yl)-5-(2,3-dihydrobenzo[b][1,4]dioxin-6-yl)-4-methyl-N-(3-(S-methylsulfonimidoyl)phenyl)nicotinamide

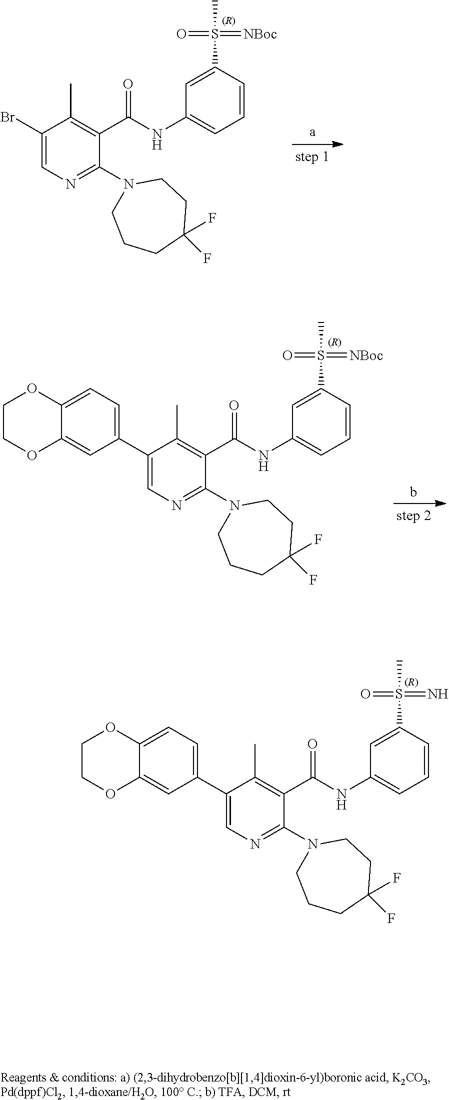

Step 1: tert-butyl (R)-((3-(2-(4,4-difluoroazepan-1-yl)-5-(2,3-dihydrobenzo[b][1,4]dioxin-6-yl)-4-methylnicotinamido)phenyl)(methyl)(oxo)-16-sulfaneylidene)carbamate: A mixture of tert-butyl (R)-((3-(5-bromo-2-(4,4-difluoroazepan-1-yl)-4-methylnicotinamido)phenyl)(methyl)(oxo)-λ⁶-sulfaneylidene)carbamate (200 mg, 0.33 mmol), (2,3-dihydrobenzo[b][1,4]dioxin-6-yl)boronic acid (120 mg, 0.67 mmol), K₂CO₃ (93 mg, 0.67 mmol) and Pd(dppf)Cl₂ (22 mg, 0.03 mmol) in dioxane (8 mL) and H₂O (2 mL) was heated at 100° C. for 16 hours under an atmosphere of N₂. After the reaction was completed, the mixture was cooled to room temperature. The resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtOAc=1/1 to 1/2) to give tert-butyl (R)-((3-(2-(4,4-difluoroazepan-1-yl)-5-(2,3-dihydrobenzo[b][1,4]dioxin-6-yl)-4-methylnicotinamido)phenyl)(methyl)(oxo)-λ⁶-sulfaneylidene)carbamate (190 mg, 87.7%) as a yellow solid. LCMS (ESI) calcd. for C₃₃H₃₉F₂N₄O₆S [M+H]⁺ m/z 657.26, found 657.15

Step 2: (R)-2-(4,4-difluoroazepan-1-yl)-5-(2,3-dihydrobenzo[b][1,4]dioxin-6-yl)-4-methyl-N-(3-(S-methylsulfonimidoyl)phenyl)nicotinamide: A solution of tert-butyl (R)-((3-(2-(4,4-difluoroazepan-1-yl)-5-(2,3-dihydrobenzo[b][1,4]dioxin-6-yl)-4-methylnicotinamido)phenyl)(methyl)(oxo)-λ⁶-sulfaneylidene)carbamate (190 mg, 0.29 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The half of residue was purified by prep-HPLC (Gemini 5 um C₁₈ column, 150*21.2 mm, eluting with 50% to 90% MeCN/H₂O containing 0.05% NH₃) to provide (R)-2-(4,4-difluoroazepan-1-yl)-5-(2,3-dihydrobenzo[b][1,4]dioxin-6-yl)-4-methyl-N-(3-(S-methylsulfonimidoyl)phenyl)nicotinamide (23.1 mg, 29%) as a white solid. ¹H NMR (400 MHz, DMSO-d₆) δ 10.84 (s, 1H), 8.38 (s, 1H), 8.03 (s, 1H), 7.90 (d, J=8.3 Hz, 1H), 7.71-7.52 (m, 2H), 6.93 (d, J=8.2 Hz, 1H), 6.86-6.72 (m, 2H), 4.28 (s, 4H), 4.21 (s, 1H), 3.68-3.49 (m, 4H), 3.05 (s, 3H), 2.31-2.20 (m, 2H), 2.15 (s, 3H), 2.07-1.95 (m, 2H), 1.84-1.76 (m, 2H). LCMS (ESI) calcd. for C₂₈H₃₁F₂N₄O₄S [M+H]⁺ m/z 557.21, found 557.15.

### Example 48

### (R)-2-(4,4-difluoroazepan-1-yl)-5-(2,3-dihydrobenzo[b][1,4]dioxin-6-yl)-4-methyl-N-(3-(S-methyl-N-(methylglycyl)sulfonimidoyl)phenyl)nicotinamide

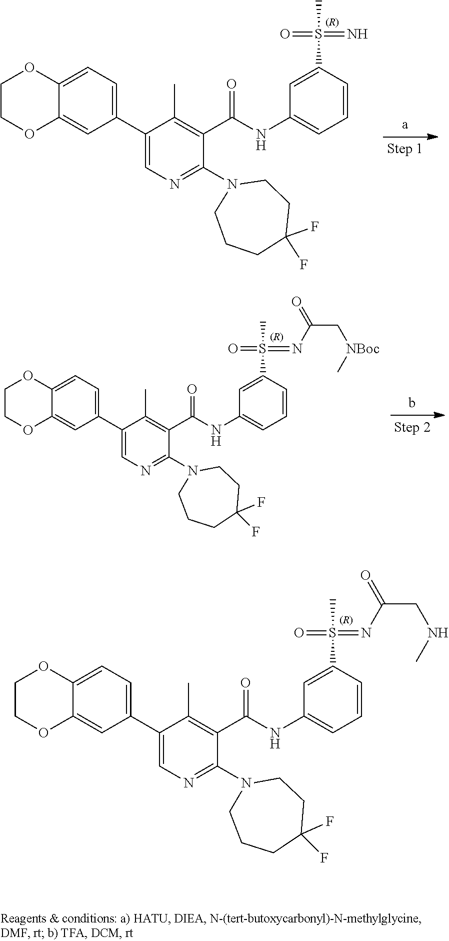

Step 1: tert-butyl (R)-(2-(((3-(2-(4,4-difluoroazepan-1-yl)-5-(2,3-dihydrobenzo[b][1,4]dioxin-6-yl)-4-methylnicotinamido)phenyl)(methyl)(oxo)-λ⁶-sulfaneylidene)amino)-2-oxoethyl)(methyl)carbamate: A mixture of (R)-2-(4,4-difluoroazepan-1-yl)-5-(2,3-dihydrobenzo[b][1,4]dioxin-6-yl)-4-methyl-N-(3-(S-methylsulfonimidoyl)phenyl)nicotinamide(100 mg, 0.18 mmol) and N-(tert-butoxycarbonyl)-N-methylglycine (41 mg, 0.22 mmol) in DMF (10 mL) was added HATU (103 mg, 0.27 mmol) and DIEA (70 mg, 0.54 mmol). The mixture was stirred at room temperature for 4 hours. The reaction was monitored by LCMS. After the reaction was completed, the resulting solution was diluted with water (20 mL) and extracted with DCM (20 mL×3). The combined organic phases were washed with brine, dried over Na₂SO₄, concentrated under vacuum. The residue was purified by flash column chromatography on silica gel (PE/EtOAc=2/1 to 1/1) to give tert-butyl (R)-(2-(((3-(2-(4,4-difluoroazepan-1-yl)-5-(2,3-dihydrobenzo[b][1,4]dioxin-6-yl)-4-methylnicotinamido)phenyl)(methyl)(oxo)-λ⁶-sulfaneylidene)amino)-2-oxoethyl)(methyl)carbamate (75 mg, 57.2%) as a yellow solid. LCMS (ESI) calcd. for C₃₆H₄₄F₂N₅O₇S [M+H]⁺ m/z 728.30, found 728.25.

Step 2: (R)-2-(4,4-difluoroazepan-1-yl)-5-(2,3-dihydrobenzo[b][1,4]dioxin-6-yl)-4-methyl-N-(3-(S-methyl-N-(methylglycyl)sulfonimidoyl)phenyl)nicotinamide: A solution of tert-butyl (R)-(2-(((3-(2-(4,4-difluoroazepan-1-yl)-5-(2,3-dihydrobenzo[b][1,4]dioxin-6-yl)-4-methylnicotinamido)phenyl)(methyl)(oxo)-λ⁶-sulfaneylidene)amino)-2-oxoethyl)(methyl)carbamate (75 mg, 0.10 mmol) in DCM (5 mL) was added TFA (0.5 mL) at room temperature. The reaction mixture was stirred at room temperature for 2 hours. After the reaction was completed, the solution was diluted with water (10 mL) and extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 50% to 95% MeCN/H₂O containing 0.05% NH₃) to obtain (R)-2-(4,4-difluoroazepan-1-yl)-5-(2,3-dihydrobenzo[b][1,4]dioxin-6-yl)-4-methyl-N-(3-(S-methyl-N-(methylglycyl)sulfonimidoyl)phenyl)nicotinamide (30.3 mg, 48.2%) as a white solid. ¹H NMR (400 MHz, DMSO-d₆) δ 10.94 (s, 1H), 8.39 (s, 1H), 8.10-7.91 (m, 2H), 7.72-7.60 (m, 2H), 6.93 (d, J=8.2 Hz, 1H), 6.85-6.72 (m, 2H), 4.28 (s, 4H), 3.68-3.51 (m, 4H), 3.45 (s, 3H), 3.19 (s, 2H), 2.35-2.19 (m, 5H), 2.15 (s, 3H), 2.07-1.94 (m, 2H), 1.84-1.76 (m, 2H). LCMS (ESI) calcd. for C₃₁H₃₆F₂N₅O₅S [M+H]⁺ m/z 628.24, found 628.15.

### Example 49

### (R)-5-(4-cyanophenyl)-2-(4,4-difluoroazepan-1-yl)-4-methyl-N-(3-(S-methylsulfonimidoyl)phenyl)nicotinamide

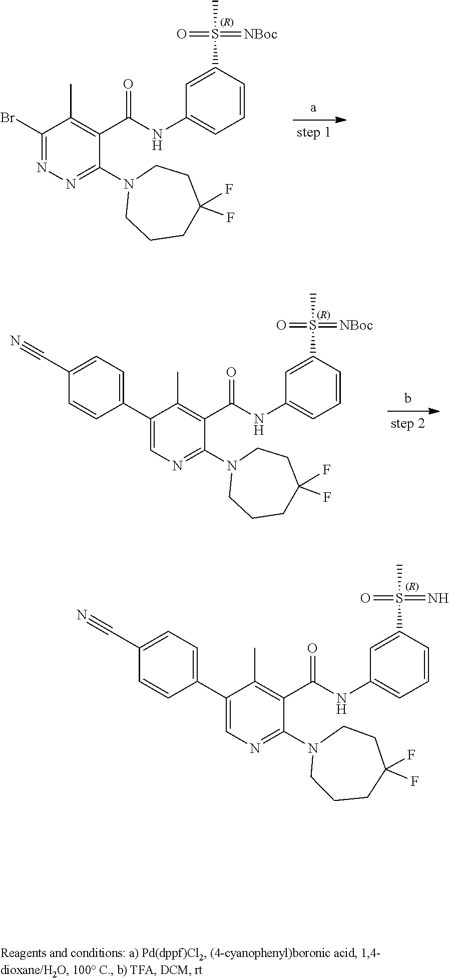

Step 1: tert-butyl (R)-((3-(5-(4-cyanophenyl)-2-(4,4-difluoroazepan-1-yl)-4-methylnicotinamido)phenyl)(methyl)(oxo)-λ⁶-sulfaneylidene)carbamate: A mixture of tert-butyl (R)-((3-(5-bromo-2-(4,4-difluoroazepan-1-yl)-4-methylnicotinamido)phenyl)(methyl)(oxo)-λ⁶-sulfaneylidene)carbamate (500 mg, 0.83 mmol), (4-cyanophenyl)boronic acid (380 mg, 2.50 mmol), potassium carbonate (345 mg, 2.50 mmol) and [1,1′-Bis(diphenylphosphino)ferrocene]dichloropalladium (II) (130 mg, 0.17 mmol) in 1,4-dioxane/H₂O (4/1, 50 mL) was heated at 100° C. for 6 hours under an atmosphere of N₂. After the reaction was completed, the mixture was diluted with water (200 mL) and extracted with DCM (80 mL×3). The combined organic phases were washed with brine, dried over Na₂SO₄, and concentrated under vacuum. The residue was purified by flash column chromatography on silica gel (PE/EtOAc=1/1) to give tert-butyl (R)-((3-(5-(4-cyanophenyl)-2-(4,4-difluoroazepan-1-yl)-4-methylnicotinamido)phenyl)(methyl)(oxo)-λ⁶-sulfaneylidene)carbamate (510 mg, 98%) as a yellow solid. LCMS (ESI) calcd. for C₃₂H₃₆F₂N₅O₄S [M+H]⁺ m/z 624.24, found 624.20.

Step 2: (R)-5-(4-cyanophenyl)-2-(4,4-difluoroazepan-1-yl)-4-methyl-N-(3-(S-methylsulfonimidoyl)phenyl)nicotinamide: A solution of tert-butyl (R)-((3-(5-(4-cyanophenyl)-2-(4,4-difluoroazepan-1-yl)-4-methylnicotinamido)phenyl)(methyl)(oxo)-λ⁶-sulfaneylidene)carbamate (510 mg, 0.82 mmol) in DCM (10 mL) was added TFA (1 mL) at room temperature. The reaction mixture was stirred at room temperature for 2 hours. After the reaction was completed, the solution was quenched with saturated aqueous NaHCO₃ (50 mL) and extracted with DCM (50 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.1% formic acid) to obtain (R)-5-(4-cyanophenyl)-2-(4,4-difluoroazepan-1-yl)-4-methyl-N-(3-(S-methylsulfonimidoyl)phenyl)nicotinamide (245 mg, 57%) as a white solid. ¹H NMR (400 MHz, DMSO-d₆) δ 10.89 (s, 1H), 8.36 (s, 1H), 8.13 (s, 1H), 7.93-7.89 (m, 3H), 7.67 (d, J=7.6 Hz, 1H), 7.60-7.56 (m, 3H), 4.22 (s, 1H), 3.68-3.66 (m, 2H), 3.61-3.58 (m, 2H), 3.06 (s, 3H), 2.32-2.29 (m, 2H), 2.16 (s, 3H), 2.02-2.00 (m, 2H), 1.83-1.82 (m, 2H). LCMS (ESI) calcd. for C₂₇H₂₈F₂N₅O₂S [M+H]⁺ m/z 524.19, found 524.15.

### Example 50

### (R)-5-(4-cyanophenyl)-2-(4,4-difluoroazepan-1-yl)-4-methyl-N-(3-(S-methyl-N-(methylglycyl)sulfonimidoyl)phenyl)nicotinamide

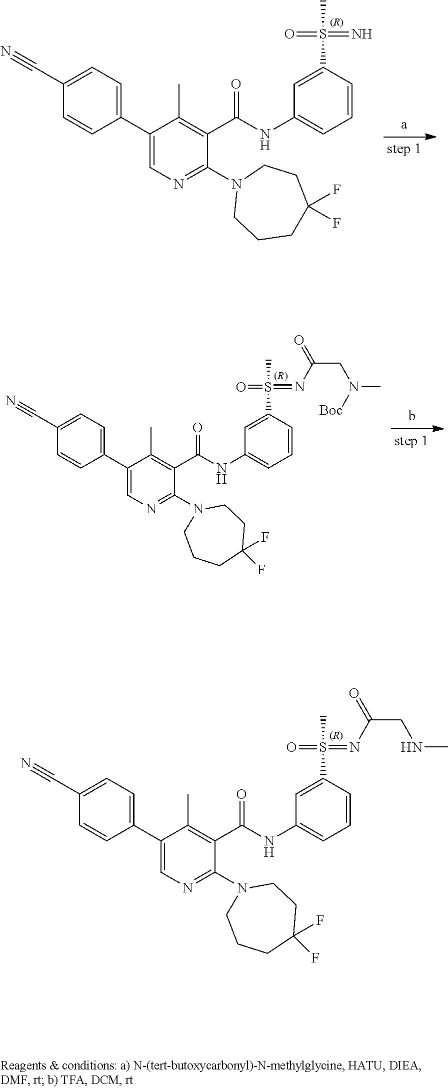

Step 1: tert-butyl (R)-(2-(((3-(5-(4-cyanophenyl)-2-(4,4-difluoroazepan-1-yl)-4-methylnicotinamido)phenyl)(methyl)(oxo)-λ⁶-sulfaneylidene)amino)-2-oxoethyl)(methyl)carbamate: A mixture of (R)-5-(4-cyanophenyl)-2-(4,4-difluoroazepan-1-yl)-4-methyl-N-(3-(S-methylsulfonimidoyl)phenyl)nicotinamide (130 mg, 0.25 mmol), N-(tert-butoxycarbonyl)-N-methylglycine (94.5 mg, 0.5 mmol) and HATU (141.6 mg, 0.37 mmol) in DMF (3 mL) was added DIEA (96.3 mg, 0.74 mmol) at room temperature. The reaction mixture was stirred at room temperature for 4 hours. After the reaction was completed, the resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtAOc=1/1) to give tert-butyl (R)-(2-(((3-(5-(4-cyanophenyl)-2-(4,4-difluoroazepan-1-yl)-4-methylnicotinamido)phenyl)(methyl)(oxo)-λ⁶-sulfaneylidene)amino)-2-oxoethyl)(methyl)carbamate (100 mg, 57.6%) as a yellow solid. LCMS (ESI) calcd. for C₃₅H₄₁F₂N₆O₅S [M+H]⁺ m/z 695.28, found 695.25.

Step 2: (R)-5-(4-cyanophenyl)-2-(4,4-difluoroazepan-1-yl)-4-methyl-N-(3-(S-methyl-N-(methylglycyl)sulfonimidoyl)phenyl)nicotinamide: A solution of tert-butyl (R)-(2-(((3-(5-(4-cyanophenyl)-2-(4,4-difluoroazepan-1-yl)-4-methylnicotinamido)phenyl)(methyl)(oxo)-λ⁶-sulfaneylidene)amino)-2-oxoethyl)(methyl)carbamate (100 mg, 0.144 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.05% NH₃) to provide (R)-5-(4-cyanophenyl)-2-(4,4-difluoroazepan-1-yl)-4-methyl-N-(3-(S-methyl-N-(methylglycyl)sulfonimidoyl)phenyl)nicotinamide (55.3 mg, 64.6%) as a white solid. ¹H NMR (400 MHz, DMSO-d₆) δ 10.98 (s, 1H), 8.38 (s, 1H), 8.14 (s, 1H), 8.00-7.96 (m, 1H), 7.93 (d, J=8.1 Hz, 2H), 7.68-7.63 (m, 2H), 7.58 (d, J=8.1 Hz, 2H), 3.68 (d, J=4.5 Hz, 2H), 3.59 (t, J=6.0 Hz, 2H), 3.45 (s, 3H), 3.19 (s, 2H), 2.31 (d, J=7.6 Hz, 2H), 2.23 (s, 3H), 2.16 (s, 3H), 2.06-1.96 (m, 2H), 1.86-1.80 (m, 2H). LCMS (ESI) calcd. for C₃₀H₃₃F₂N₆O₃S [M+H]⁺ m/z 595.23, found 595.15.

### Example 51

### (R)-N-(3-(N-(azetidine-3-carbonyl)-S-methylsulfonimidoyl)phenyl)-2-(4,4-difluoroazepan-1-yl)-4-methyl-5-(1-methyl-1H-pyrazol-4-yl)nicotinamide formate

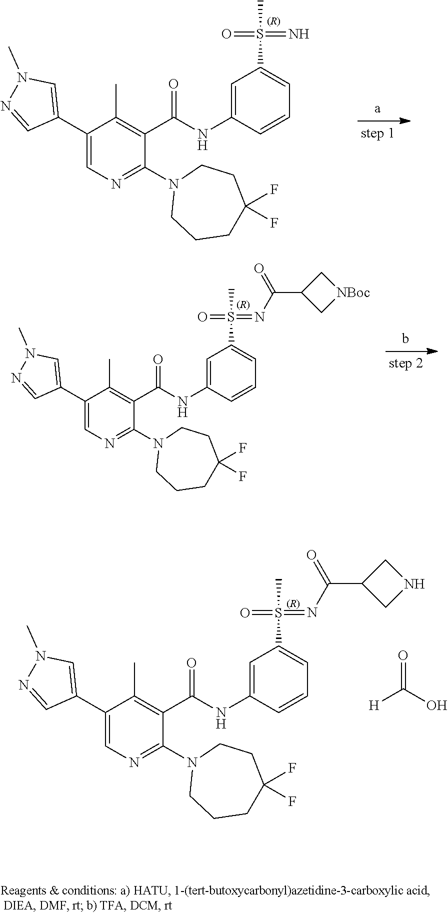

Step 1: tert-butyl (R)-3-(((3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)carbamoyl)azetidine-1-carboxylate: To a mixture of (R)-2-(4,4-difluoroazepan-1-yl)-4-methyl-5-(1-methyl-1H-pyrazol-4-yl)-N-(3-(S-methylsulfonimidoyl)phenyl)nicotinamide (100 mg, 0.199 mmol), 1-(tert-butoxycarbonyl)azetidine-3-carboxylic acid (80 mg, 0.398 mmol) and HATU (151 mg, 0.398 mmol) in DMF (5 mL) was added DIEA (77 mg, 0.597 mmol). The mixture was stirred at room temperature for 2 hours. After the reaction was completed, the resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtOAc=1/4) to give tert-butyl (R)-3-(((3-(2-(4,4-difluoroazepan-1-yl)-4-methyl-5-(1-methyl-1H-pyrazol-4-yl)nicotinamido)phenyl)(methyl)(oxo)-λ⁶--sulfaneylidene)carbamoyl)azetidine-1-carboxylate (92 mg, 67.65%) as a yellow oil. LCMS (ESI) calcd. for C₃₃H₄₂F₂N₇O₅S [M+H]⁺ m/z 686.30, found 686.20.

Step 2: (R)-N-(3-(N-(azetidine-3-carbonyl)-S-methylsulfonimidoyl)phenyl)-2-(4,4-difluoroazepan-1-yl)-4-methyl-5-(1-methyl-1H-pyrazol-4-yl)nicotinamide formate: A solution of tert-butyl (R)-3-(((3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)carbamoyl)azetidine-1-carboxylate (92 mg, 0.134 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.1% formic acid) to provide (R)-N-(3-(N-(azetidine-3-carbonyl)-S-methylsulfonimidoyl)phenyl)-2-(4,4-difluoroazepan-1-yl)-4-methyl-5-(1-methyl-1H-pyrazol-4-yl)nicotinamide formate (45 mg, 57.69%) as a white solid. ¹H NMR (400 MHz, DMSO-d₆) δ 11.25 (s, 1H), 8.34 (s, 1H), 8.24 (s, 1H), 8.17 (s, 1H), 8.09 (d, J=7.3 Hz, 1H), 7.90 (s, 1H), 7.73-7.65 (m, 2H), 7.61 (s, 1H), 3.98-3.78 (m, 8H), 3.63-3.53 (m, 5H), 3.48 (s, 3H), 2.33-2.18 (m, 5H), 2.07-1.94 (m, 2H), 1.85-1.74 (m, 2H). LCMS (ESI) calcd. for C₂₈H₃₄F₂N₇O₃S [M+H]⁺ m/z 586.24, found 586.15.

### Example 52

### (R)-N-(3-(N-(2-amino-2-methylpropanoyl)-S-methylsulfonimidoyl)phenyl)-2-(4,4-difluoroazepan-1-yl)-4-methyl-5-(1-methyl-1H-pyrazol-4-yl)nicotinamide formate

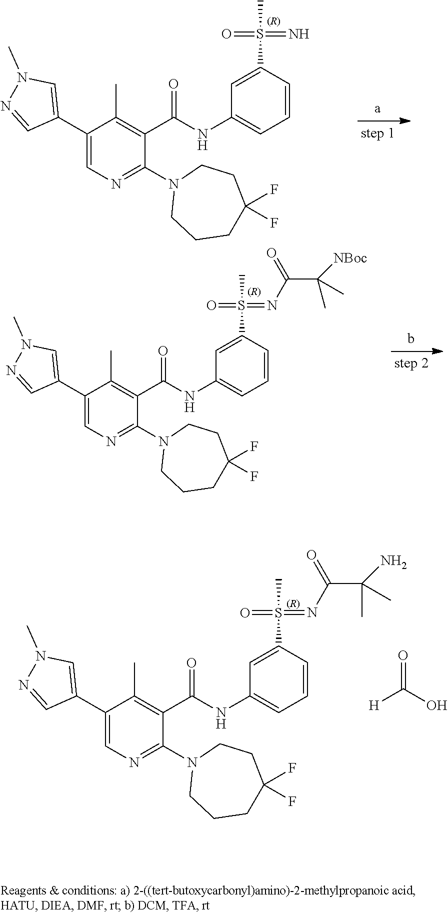

Step 1: tert-butyl (R)-(1-(((3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)amino)-2-methyl-1-oxopropan-2-yl)carbamate: To a mixture of (R)-2-(4,4-difluoroazepan-1-yl)-4-methyl-5-(1-methyl-1H-pyrazol-4-yl)-N-(3-(S-methylsulfonimidoyl)phenyl)nicotinamide (100 mg, 0.20 mmol), 2-((tert-butoxycarbonyl)amino)-2-methylpropanoic acid (81 mg, 0.40 mmol) and HATU (151 mg, 0.40 mmol) in DMF (5 mL) was added DIEA (77 mg, 0.60 mmol). The mixture was stirred at 50° C. for 16 hours. After the reaction was completed, the resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtOAc=1/1 to 1/4) to give tert-butyl (R)-(1-(((3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)amino)-2-methyl-1-oxopropan-2-yl)carbamate (85 mg, 62.04%) as a yellow oil. LCMS (ESI) calcd. for C₃₃H₄₄F₂N₇O₅S [M+H]⁺ m/z 688.31, found 688.25.

Step 2: (R)-N-(3-(N-(2-amino-2-methylpropanoyl)-S-methylsulfonimidoyl)phenyl)-2-(4,4-difluoroazepan-1-yl)-4-methyl-5-(1-methyl-1H-pyrazol-4-yl)nicotinamide formate: A solution of tert-butyl (R)-(1-(((3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)amino)-2-methyl-1-oxopropan-2-yl)carbamate (85 mg, 0.12 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.1% formic acid) to provide (R)-N-(3-(N-(2-amino-2-methylpropanoyl)-S-methylsulfonimidoyl)phenyl)-2-(4,4-difluoroazepan-1-yl)-4-methyl-5-(1-methyl-1H-pyrazol-4-yl)nicotinamide formate (30.5 mg, 43.6%) as a white solid. ¹H NMR (400 MHz, DMSO-d₆) δ 11.01 (s, 1H), 8.44 (d, J=1.6 Hz, 1H), 8.33 (d, J=3.7 Hz, 1H), 8.18 (d, J=2.6 Hz, 1H), 8.03-7.87 (m, 2H), 7.75-7.57 (m, 3H), 3.88 (s, 3H), 3.63-3.58 (m, 2 H), 3.56-3.52 (m, 2H), 3.49 (s, 3H), 2.34-2.21 (m, 5H), 2.09-1.93 (m, 2H), 1.87-1.74 (m, 2H), 1.40-1.27 (m, 6H). LCMS (ESI) calcd. for C₂₈H₃₆F₂N₇O₃S [M+H]⁺ m/z 588.26, found 588.20.

### Example 53

### (R)-N-(3-(N-(1-aminocyclopropane-1-carbonyl)-S-methylsulfonimidoyl)phenyl)-2-(4,4-difluoroazepan-1-yl)-4-methyl-5-(1-methyl-1H-pyrazol-4-yl)nicotinamide formate

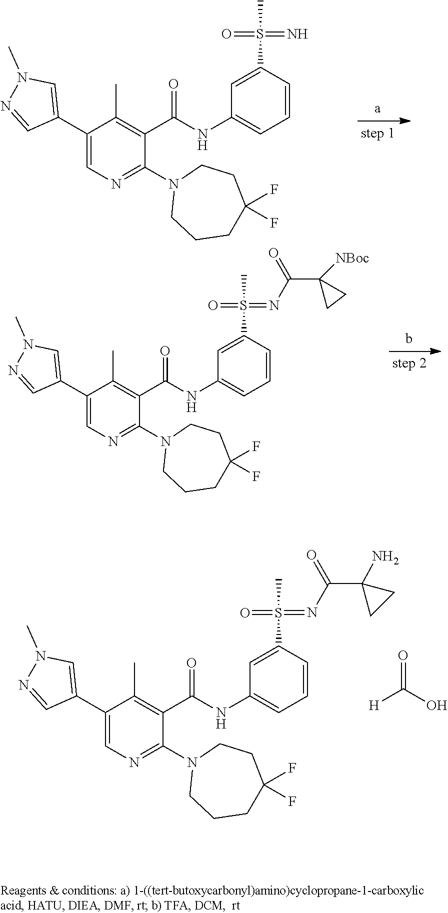

Step 1: tert-butyl (R)-(1-(((3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)carbamoyl)cyclopropyl)carbamate: To a mixture of (R)-2-(4,4-difluoroazepan-1-yl)-4-methyl-5-(1-methyl-1H-pyrazol-4-yl)-N-(3-(S-methylsulfonimidoyl)phenyl)nicotinamide (100 mg, 0.20 mmol), 1-((tert-butoxycarbonyl)amino)cyclopropane-1-carboxylic acid (80 mg, 0.40 mmol) and HATU (151 mg, 0.40 mmol) in DMF (5 mL) was added DIEA (77 mg, 0.60 mmol). The mixture was stirred at 50° C. for 16 hours. After the reaction was completed, the resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtOAc=1/4) to give tert-butyl (R)-(1-(((3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)carbamoyl)cyclopropyl)carbamate (85 mg, 62.5%) as a yellow oil. LCMS (ESI) calcd. for C₃₃H₄₂F₂N₇O₅S [M+H]⁺ m/z 686.30, found 686.25.

Step 2: (R)-N-(3-(N-(1-aminocyclopropane-1-carbonyl)-S-methylsulfonimidoyl)phenyl)-2-(4,4-difluoroazepan-1-yl)-4-methyl-5-(1-methyl-1H-pyrazol-4-yl)nicotinamide formate: A solution of tert-butyl (R)-(1-(((3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)carbamoyl)cyclopropyl)carbamate (85 mg, 0.12 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.1% formic acid) to provide (R)-N-(3-(N-(1-aminocyclopropane-1-carbonyl)-S-methylsulfonimidoyl)phenyl)-2-(4,4-difluoroazepan-1-yl)-4-methyl-5-(1-methyl-1H-pyrazol-4-yl)nicotinamide formate (52.3 mg, 72.1%) as a white solid. ¹H NMR (400 MHz, DMSO-d₆) δ 10.95 (s, 1H), 8.38 (s, 1H), 8.18 (s, 2H), 7.98 (d, J=5.9 Hz, 1H), 7.90 (s, 1H), 7.69-7.62 (m, 2H), 7.61 (s, 1H), 3.89 (s, 3H), 3.65-3.59 (m, 2H), 3.55 (t, J=5.9 Hz, 2H), 3.44 (s, 3H), 2.32-2.15 (m, 5H), 2.06-1.91 (m, 2H), 1.85-1.76 (m, 2H), 1.29-1.17 (m, 2H), 0.87-0.79 (m, 2H). LCMS (ESI) calcd. for C₂₈H₃₄F₂N₇O₃S [M+H]⁺ m/z 586.24, found 586.20.

### Example 54

### (R)-2-(4,4-difluoroazepan-1-yl)-4-methyl-5-(1-methyl-1H-pyrazol-4-yl)-N-(3-(S-methyl-N-(oxetane-3-carbonyl)sulfonimidoyl)phenyl)nicotinamide

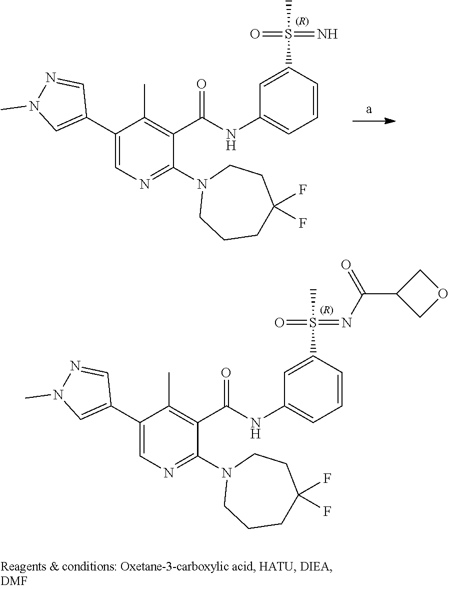

To a mixture of (R)-2-(4,4-difluoroazepan-1-yl)-4-methyl-5-(1-methyl-1H-pyrazol-4-yl)-N-(3-(S-methylsulfonimidoyl)phenyl)nicotinamide (120 mg, 0.24 mmol), oxetane-3-carboxylic acid (49 mg, 0.48 mmol) and HATU (182 mg, 0.48 mmol) in DMF (5 mL) was added DIEA (92 mg, 0.72 mmol). The mixture was stirred at room temperature for 2 hours. After the reaction was completed, the resulting solution was diluted with water (50 mL) and extracted with EtOAc (30 mL×3). The combined organic layers were washed with brine, dried over Na₂SO₄, concentrated under vacuum. The residue was purified by flash column chromatography on silica gel (PE/EtOAc=1/4) to give the crude product. Then the residue was purified by prep-HPLC (Gemini 5 um C₁₈ column, 150*21.2 mm, eluting with 50% to 95% MeCN/H₂O containing 0.1% formic acid) to provide (R)-2-(4,4-difluoroazepan-1-yl)-4-methyl-5-(1-methyl-1H-pyrazol-4-yl)-N-(3-(S-methyl-N-(oxetane-3-carbonyl)sulfonimidoyl)phenyl)nicotinamide (85.2 mg, 60.87%) as a yellow oil. ¹H NMR (400 MHz, DMSO-d₆) δ 10.96 (s, 1H), 8.41 (s, 1H), 8.18 (s, 1H), 7.97 (d, J=6.4 Hz, 1H), 7.90 (s, 1H), 7.71-7.64 (m, 2H), 7.61 (s, 1H), 4.71-4.58 (m, 4H), 3.88 (s, 3H), 3.85-3.77 (m, 1H), 3.63-3.58 (m, 2H), 3.54 (t, J=5.9 Hz, 2H), 3.48 (s, 3H), 2.31-2.19 (m, 5H), 2.06-1.93 (m, 2H), 1.85-1.74 (m, 2H). LCMS (ESI) calcd. for C₂₈H₃₃F₂N₆O₄S [M+H]⁺ m/z 587.23, found 587.15.

### Example 55

### (R)-N-(3-(N-(2-amino-2-methylpropanoyl)-S-methylsulfonimidoyl)phenyl)-5-(4-cyanophenyl)-2-(4,4-difluoroazepan-1-yl)-4-methylnicotinamide

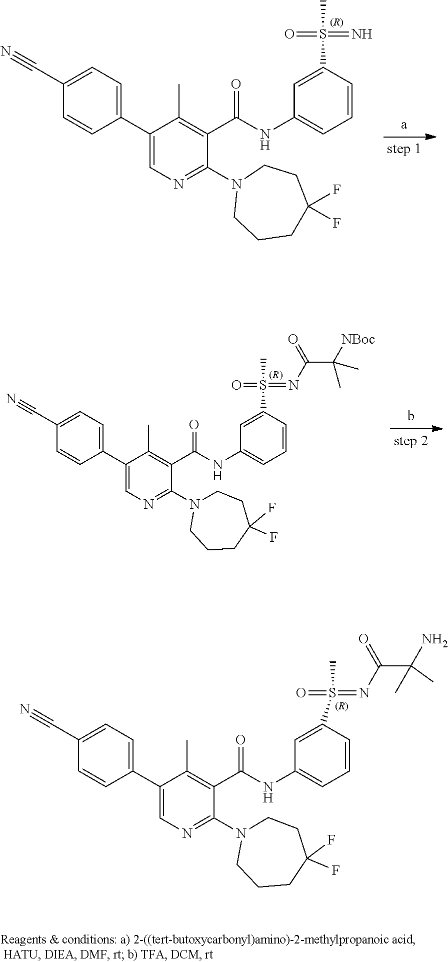

Step 1: tert-butyl (R)-(1-(((3-(5-(4-cyanophenyl)-2-(4,4-difluoroazepan-1-yl)-4-methylnicotinamido)phenyl)(methyl)(oxo)-λ⁶-sulfaneylidene)amino)-2-methyl-1-oxopropan-2-yl)carbamate: A mixture of (R)-5-(4-cyanophenyl)-2-(4,4-difluoroazepan-1-yl)-4-methyl-N-(3-(S-methylsulfonimidoyl)phenyl)nicotinamide (100 mg, 0.19 mmol), 2-((tert-butoxycarbonyl)amino)-2-methylpropanoic acid (77.6 mg, 0.38 mmol) and HATU (109 mg, 0.29 mmol) in DMF (3 mL) was added DIEA (74 mg, 0.57 mmol) at room temperature. The reaction mixture was stirred at 50° C. for 16 hours. After the reaction was completed, the resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtAOc=1/1) to give tert-butyl (R)-(1-(((3-(5-(4-cyanophenyl)-2-(4,4-difluoroazepan-1-yl)-4-methylnicotinamido)phenyl)(methyl)(oxo)-λ⁶-sulfaneylidene)amino)-2-methyl-1-oxopropan-2-yl)carbamate (95 mg, 70.6%) as a yellow solid. LCMS (ESI) calcd. for C₃₆H₄₃F₂N₆O₅S [M+H]⁺ m/z 709.30, found 709.20.

Step 2: (R)-N-(3-(N-(2-amino-2-methylpropanoyl)-S-methylsulfonimidoyl)phenyl)-5-(4-cyanophenyl)-2-(4,4-difluoroazepan-1-yl)-4-methylnicotinamide: A solution of tert-butyl (R)-(1-(((3-(5-(4-cyanophenyl)-2-(4,4-difluoroazepan-1-yl)-4-methylnicotinamido)phenyl)(methyl)(oxo)-λ⁶-sulfaneylidene)amino)-2-methyl-1-oxopropan-2-yl)carbamate (95 mg, 0.13 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.05% NH₃) to provide (R)-N-(3-(N-(2-amino-2-methylpropanoyl)-S-methylsulfonimidoyl)phenyl)-5-(4-cyanophenyl)-2-(4,4-difluoroazepan-1-yl)-4-methylnicotinamide (41.3 mg, 67.9%) as a white solid. ¹H NMR (400 MHz, DMSO-d₆) δ 10.98 (s, 1H), 8.41 (s, 1H), 8.14 (s, 1H), 7.93 (d, J=8.4 Hz, 3H), 7.69-7.64 (m, 2H), 7.58 (d, J=8.3 Hz, 2H), 3.71-3.64 (m, 2H), 3.59 (t, J=6.0 Hz, 2H), 3.43 (s, 3H), 2.36-2.27 (m, 2H), 2.16 (s, 3H), 2.06-1.96 (m, 2H), 1.87-1.67 (m, 4H), 1.20 (d, J=3.6 Hz, 6H). LCMS (ESI) calcd. for C₃₁H₃₅F₂N₆O₃S [M+H]⁺ m/z 609.25, found 609.20.

### Example 56

### (R)-5-cyano-2-(4,4-difluoroazepan-1-yl)-4-methyl-N-(3-(S-methylsulfonimidoyl)phenyl)-6-(trifluoromethyl)nicotinamide

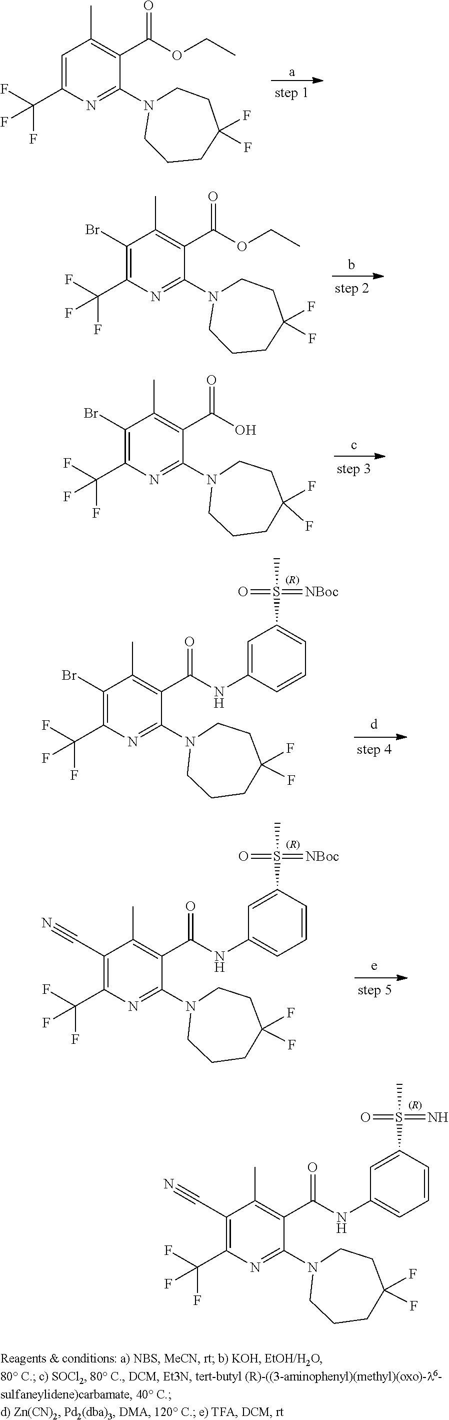

Step 1: ethyl 5-bromo-2-(4,4-difluoroazepan-1-yl)-4-methyl-6-(trifluoromethyl)nicotinate: A solution of ethyl 2-(4,4-difluoroazepan-1-yl)-4-methyl-6-(trifluoromethyl)nicotinate (1.4 g, 3.82 mmol) and NBS (817 mg, 4.59 mmol) in MeCN (30 mL) was stirred at room temperature for 2 hours. LCMS showed the reaction was completed. The mixture was diluted with water (60 mL) and extracted with EtOAc (50 mL×3). The combined organic phases were washed with brine, dried over sodium sulfate, concentrated under vacuum. The residue was purified by flash column chromatography on silica gel (PE/EtOAc=5/1) to provide ethyl 5-bromo-2-(4,4-difluoroazepan-1-yl)-4-methyl-6-(trifluoromethyl)nicotinate (1.6 g, 94.1%) as yellow solid. LCMS (ESI) calcd. for C₁₆H₁₉BrF₅N₂O₂ [M+H]⁺ m/z 447.06, found 447.05.

Step 2: 5-bromo-2-(4,4-difluoroazepan-1-yl)-4-methyl-6-(trifluoromethyl)nicotinic acid: To a solution of ethyl 5-bromo-2-(4,4-difluoroazepan-1-yl)-4-methyl-6-(trifluoromethyl)nicotinate (1.6 g, 3.59 mmol) in EtOH/THF/H₂O (1/1/1, 30 mL) was added KOH (2.01 g, 35.9 mmol) at 25° C. The mixture was heated at 80° C. for 16 hours. After the reaction was completed, the mixture was concentrated to remove most EtOH/THF. The aqueous phase was adjusted to pH=3-4 with 1N HCl then extracted with EtOAc (50 mL×3). The combined organic phases were washed with brine, dried with Na₂SO₄, and concentrated under reduced pressure to afford 5-bromo-2-(4,4-difluoroazepan-1-yl)-4-methyl-6-(trifluoromethyl)nicotinic acid (1.2 g, 80.1%) as a yellow solid. LCMS (ESI) calcd. for C₁₄H₁₅BrF₅N₂O₂ [M+H]⁺ m/z 417.03, found 417.00.

Step 3: tert-butyl (R)-((3-(5-bromo-2-(4,4-difluoroazepan-1-yl)-4-methyl-6-(trifluoromethyl)nicotinamido)phenyl)(methyl)(oxo)-λ⁶-sulfaneylidene)carbamate: A solution of 5-bromo-2-(4,4-difluoroazepan-1-yl)-4-methyl-6-(trifluoromethyl)nicotinic acid (600 mg, 1.44 mmol) in SOCl₂ (6 mL) was heated to 80° C. for 0.5 h. The solution was concentrated under vacuum to provide the chloride intermediate. Then the chloride intermediate dissolved in DCM (5 mL) was added to a solution of tert-butyl (R)-((3-aminophenyl)(methyl)(oxo)-λ⁶-sulfaneylidene)carbamate (465 mg, 1.72 mmol) and TEA (436 mg, 4.30 mmol) in DCM (6 mL) at 0° C. The resulting mixture was stirred at 40° C. for 2 hours. Then the mixture was quenched with water (50 mL) and extracted with DCM (50 mL×3). The combined organic phases were washed with brine, dried over sodium sulfate, concentrated under vacuum. The residue was purified by flash column chromatography on silica gel (PE/EtOAc=1/1) to provide tert-butyl (R)-((3-(5-bromo-2-(4,4-difluoroazepan-1-yl)-4-methyl-6-(trifluoromethyl)nicotinamido)phenyl)(methyl)(oxo)-λ⁶-sulfaneylidene)carbamate (450 mg, 32.7%) as a yellow solid. LCMS (ESI) calcd. for C₂₆H₃₁BrF₅N₄O₄S [M+H]⁺ m/z 671.12, found 671.05.

Step 4: tert-butyl (R)-((3-(5-cyano-2-(4,4-difluoroazepan-1-yl)-4-methyl-6-(trifluoromethyl)nicotinamido)phenyl)(methyl)(oxo)-λ⁶-sulfaneylidene)carbamate: A mixture of tert-butyl (R)-((3-(5-bromo-2-(4,4-difluoroazepan-1-yl)-4-methyl-6-(trifluoromethyl)nicotinamido)phenyl)(methyl)(oxo)-λ⁶-sulfaneylidene)carbamate (200 mg, 0.299 mmol), Zn(CN)₂ (39 mg, 0.329 mmol), Pd₂(dba)₃ (55 mg, 0.060 mmol) in DMA (2 mL) was heated at 120° C. for 16 h under an atmosphere of N₂. After the reaction was completed, the mixture was cooled to room temperature. The resulting solution was diluted with water (30 mL) and extracted with EtOAc (30 mL×3). The combined organic phases were washed with brine, dried over Na₂SO₄, concentrated under vacuum. The residue was purified by flash column chromatography on silica gel (PE/EtOAc=1/1) to give tert-butyl (R)-((3-(5-cyano-2-(4,4-difluoroazepan-1-yl)-4-methyl-6-(trifluoromethyl)nicotinamido)phenyl)(methyl)(oxo)-λ⁶-sulfaneylidene)carbamate(110 mg, 53.8%) as a yellow solid. LCMS (ESI) calcd. for C₂₇H₃₀F5N₅O₄SNa [M+Na]⁺ m/z 618.19, found 638.15.

Step 5: (R)-5-cyano-2-(4,4-difluoroazepan-1-yl)-4-methyl-N-(3-(S-methylsulfonimidoyl)phenyl)-6-(trifluoromethyl)nicotinamide: A solution of tert-butyl (R)-((3-(5-cyano-2-(4,4-difluoroazepan-1-yl)-4-methyl-6-(trifluoromethyl)nicotinamido)phenyl)(methyl)(oxo)-λ⁶-sulfaneylidene)carbamate (110 mg, 0.179 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 35% to 80% MeCN/H₂O containing 0.1% formic acid) to provide (R)-5-cyano-2-(4,4-difluoroazepan-1-yl)-4-methyl-N-(3-(S-methylsulfonimidoyl)phenyl)-6-(trifluoromethyl)nicotinamide (72.1 mg, 76.6%) as a white solid. ¹H NMR (400 MHz, DMSO-d₆) δ 10.98 (s, 1H), 8.27 (s, 1H), 7.89 (d, J=8.2 Hz, 1H), 7.71 (d, J=7.9 Hz, 1H), 7.62 (t, J=7.9 Hz, 1H), 4.26 (s, 1H), 3.69 (t, J=25.8 Hz, 4H), 3.07 (s, 3H), 2.47 (s, 3H), 2.32 (d, J=8.9 Hz, 2H), 2.08-1.99 (m, 2H), 1.86 (s, 2H). LCMS (ESI) calcd. for C₂₂H₂₃F₅N₅O₂S [M+H]⁺ m/z 516.15, found 516.35.

### Example 57

### (R)-N-(3-(N-(azetidine-3-carbonyl)-S-methylsulfonimidoyl)phenyl)-5-cyano-2-(4,4-difluoroazepan-1-yl)-4-methyl-6-(trifluoromethyl)nicotinamide formate

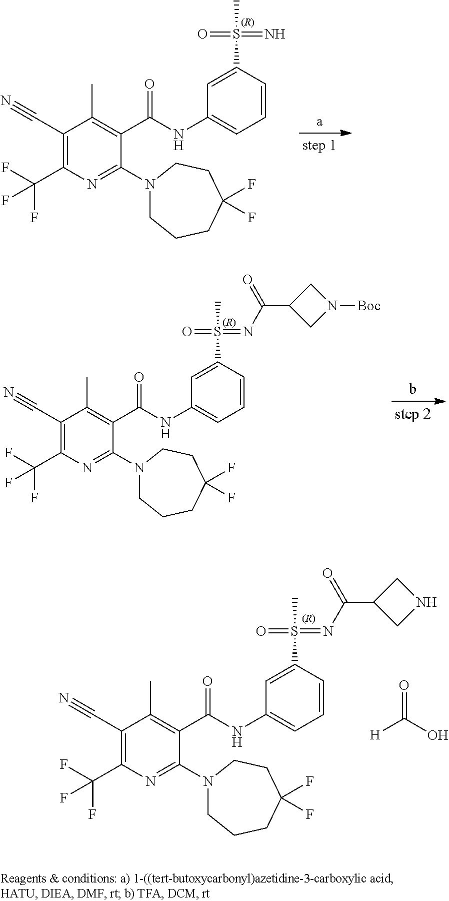

Step 1: tert-butyl (R)-3-(((3-(5-cyano-2-(4,4-difluoroazepan-1-yl)-4-methyl-6-(trifluoromethyl)nicotinamido)phenyl)(methyl)(oxo)-λ⁶-sulfaneylidene)carbamoyl) azetidine-1-carboxylate: A solution of (R)-5-cyano-2-(4,4-difluoroazepan-1-yl)-4-methyl-N-(3-(S-methylsulfonimidoyl) phenyl)-6-(trifluoromethyl)nicotinamide (95 mg, 0.18 mmol), 1-(tert-butoxycarbonyl)azetidine-3-carboxylic acid (75 mg, 0.37 mmol), HATU (140 mg, 0.37 mmol) and DIEA (71 mg, 0.55 mmol) in DMF (5 mL) was stirred at room temperature for 16 hours. LCMS showed the reaction was completed. The mixture was diluted with water (40 mL) and extracted with EtOAc (30 mL×3). The combined organic layers were washed with brine, dried over sodium sulfate, concentrated under vacuum. The residue was purified by flash column chromatography on silica gel (DCM/MeOH=95/5) to provide tert-butyl (R)-3-(((3-(5-cyano-2-(4,4-difluoroazepan-1-yl)-4-methyl-6-(trifluoromethyl)nicotinamido)phenyl)(methyl)(oxo)-λ⁶-sulfaneylidene)carbamoyl) azetidine-1-carboxylate (100 mg, 77.7%) as brown oil. LCMS (ESI) calcd. for C₃₁H₃₅F₅N₆O₅SNa [M+Na]⁺ m/z 721.22, found 721.20.

Step 2: (R)-N-(3-(N-(azetidine-3-carbonyl)-S-methylsulfonimidoyl)phenyl)-5-cyano-2-(4,4-difluoroazepan-1-yl)-4-methyl-6-(trifluoromethyl)nicotinamide formate A solution of tert-butyl (R)-3-(((3-(5-cyano-2-(4,4-difluoroazepan-1-yl)-4-methyl-6-(trifluoromethyl)nicotinamido)phenyl)(methyl)(oxo)-λ⁶-sulfaneylidene)carbamoyl)azetidine-1-carboxylate (100 mg, 0.14 mmol) in DCM (4 mL) was added TFA (0.4 mL) at room temperature. The reaction mixture was stirred at room temperature for 1 hour. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 30% to 95% MeCN/H₂O containing 0.1% formic acid) to provide (R)-N-(3-(N-(azetidine-3-carbonyl)-S-methylsulfonimidoyl)phenyl)-5-cyano-2-(4,4-difluoroazepan-1-yl)-4-methyl-6-(trifluoromethyl)nicotinamide formate (38.72 mg, 43.74%) as a white solid. ¹H NMR (400 MHz, CD₃OD) δ 8.53 (d, J=9.3 Hz, 2H), 7.88 (d, J=8.0 Hz, 1H), 7.81 (d, J=8.5 Hz, 1H), 7.69 (t, J=8.0 Hz, 1H), 4.28-4.15 (m, 4H), 3.90-3.77 (m, 2H), 3.74-3.69 (m, 3H), 3.48 (s, 3H), 2.54 (s, 3H), 2.36-2.26 (m, 2H), 2.09-1.88 (m, 4H). LCMS (ESI) calcd. for C₂₆H₂₈F₅N₆O₃S [M+H]⁺ m/z 599.19, found 599.15.

### Example 58

### (R)-N-(3-(N-(3-aminopropanoyl)-S-methylsulfonimidoyl)phenyl)-5-cyano-2-(4,4-difluoroazepan-1-yl)-4-methyl-6-(trifluoromethyl)nicotinamide formate

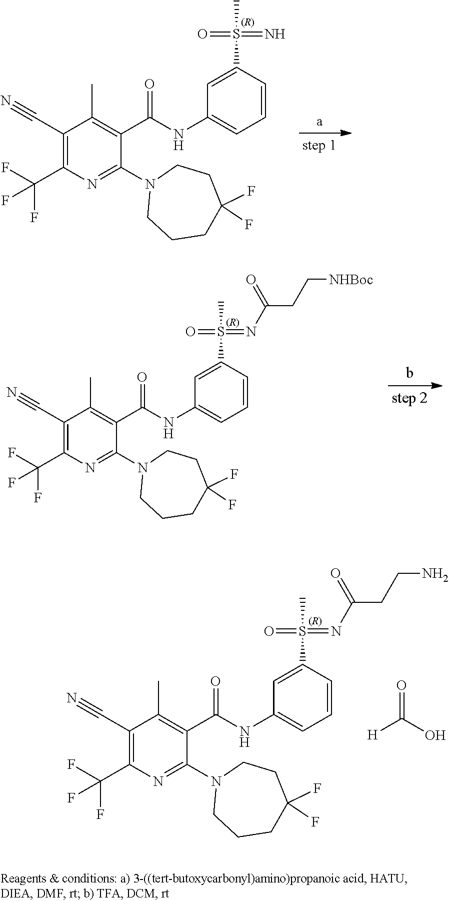

Step 1: tert-butyl (R)-(3-(((3-(5-cyano-2-(4,4-difluoroazepan-1-yl)-4-methyl-6-(trifluoromethyl)nicotinamido)phenyl)(methyl)(oxo)-λ⁶-sulfaneylidene)amino)-3-oxopropyl)carbamate: A solution of (R)-5-cyano-2-(4,4-difluoroazepan-1-yl)-4-methyl-N-(3-(S-methylsulfonimidoyl)phenyl)-6-(trifluoromethyl)nicotinamide (110 mg, 0.21 mmol), 3-((tert-butoxycarbonyl)amino)propanoic acid (61 mg, 0.32 mmol), HATU (162 mg, 0.43 mmol) and DIEA (83 mg, 0.64 mmol) in DMF (5 mL) was stirred at room temperature for 16 hours. LCMS showed the reaction was completed. The mixture was diluted with water (50 mL) and extracted with EtOAc (30 mL×3). The combined organic layers were washed with brine, dried over sodium sulfate, concentrated under vacuum. The residue was purified by flash column chromatography on silica gel (DCM/MeOH=95/5) to provide tert-butyl (R)-(3-(((3-(5-cyano-2-(4,4-difluoroazepan-1-yl)-4-methyl-6-(trifluoromethyl)nicotinamido)phenyl)(methyl)(oxo)-λ⁶-sulfaneylidene)amino)-3-oxopropyl)carbamate (100 mg, 63.2%) as brown oil. LCMS (ESI) calcd. for C₃₀H₃₆F₅N₆O₅S [M+H]⁺ m/z 687.24, found 687.10.

Step 2: (R)-N-(3-(N-(3-aminopropanoyl)-S-methylsulfonimidoyl)phenyl)-5-cyano-2-(4,4-difluoroazepan-1-yl)-4-methyl-6-(trifluoromethyl)nicotinamide formate: A solution of tert-butyl (R)-(3-(((3-(5-cyano-2-(4,4-difluoroazepan-1-yl)-4-methyl-6-(trifluoromethyl)nicotinamido)phenyl)(methyl)(oxo)-λ⁶-sulfaneylidene)amino)-3-oxopropyl)carbamate (100 mg, 0.15 mmol) in DCM (5 mL) was added TFA (0.5 mL) at room temperature. The reaction mixture was stirred at room temperature for 1 hour. After the reaction was completed, the mixture was concentrated. The residue was adjusted to pH=8-9 with saturated aqueous NaHCO₃. Then the aqueous solution was extracted with DCM (30 mL×3). The combined organic layers were washed with brine, dried over sodium sulfate, concentrated under vacuum. The residue was purified by prep-HPLC (Gemini 5 um C₁₈ column, 150*21.2 mm, eluting with 30% to 95% MeCN/H₂O containing 0.1% formic acid) to provide (R)-N-(3-(N-(3-aminopropanoyl)-S-methylsulfonimidoyl)phenyl)-5-cyano-2-(4,4-difluoroazepan-1-yl)-4-methyl-6-(trifluoromethyl)nicotinamide formate (31.3 mg, 36.2%) as a white solid. ¹H NMR (400 MHz, CD₃OD) δ 8.53 (s, 2H), 7.91-7.84 (m, 1H), 7.83-7.86 (m, 1H), 7.69 (t, J=8.0 Hz, 1H), 3.84 (s, 2H), 3.77-3.71 (m, 2H), 3.47 (s, 3H), 3.15 (t, J=6.5 Hz, 2H), 2.76 (t, J=6.5 Hz, 2H), 2.55 (s, 3H), 2.39-2.25 (m, 2H), 2.11-1.87 (m, 4H). LCMS (ESI) calcd. for C₂₅H₂₈F₅N₆O₃S [M+H]⁺ m/z 587.19, found 586.95.

### Example 59

### (R)-2-(4,4-difluoroazepan-1-yl)-5-(3,4-difluorophenyl)-4-methyl-N-(3-(S-methylsulfonimidoyl)phenyl)nicotinamide

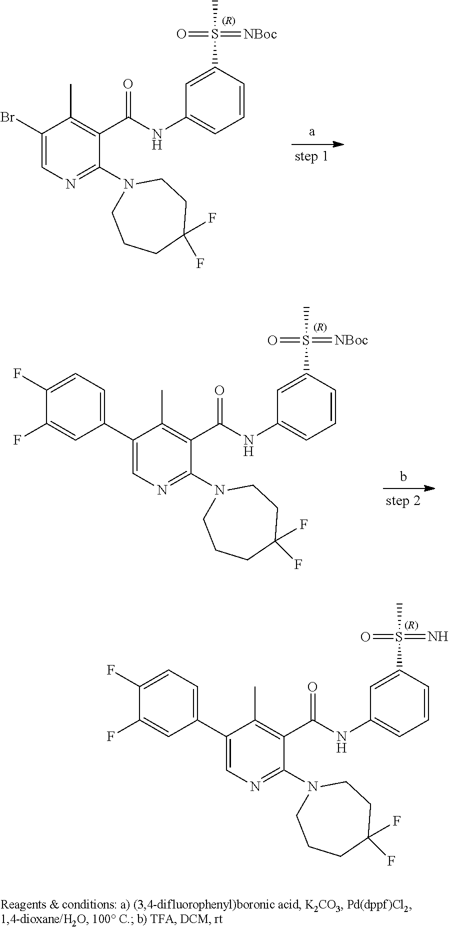

Step 1: tert-butyl (R)-((3-(2-(4,4-difluoroazepan-1-yl)-5-(3,4-difluorophenyl)-4-methylnicotinamido)phenyl)(methyl)(oxo)-λ⁶-sulfaneylidene)carbamate: A mixture of tert-butyl (R)-((3-(5-bromo-2-(4,4-difluoroazepan-1-yl)-4-methylnicotinamido)phenyl)(methyl)(oxo)-λ⁶-sulfaneylidene)carbamate (100 mg, 0.17 mmol), (3,4-difluorophenyl)boronic acid (52.5 mg, 0.33 mmol), K₂CO₃ (68.9 mg, 0.5 mmol) and Pd(dppf)Cl₂ (24.3 mg, 0.03 mmol) in 1,4-dioxane/H₂O (4/1, 5 mL) was heated at 100° C. for 5 hours under an atmosphere of N₂. After the reaction was completed, the mixture was cooled to room temperature. The resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtAOc=1/1) to give tert-butyl (R)-((3-(2-(4,4-difluoroazepan-1-yl)-5-(3,4-difluorophenyl)-4-methylnicotinamido)phenyl)(methyl)(oxo)-λ⁶-sulfaneylidene)carbamate (86 mg, 81.6%) as a yellow oil. LCMS (ESI) calcd. for C₃₁H₃₅F₄N₄O₄S [M+H]⁺ m/z 635.23, found 635.10.

Step 2: (R)-2-(4,4-difluoroazepan-1-yl)-5-(3,4-difluorophenyl)-4-methyl-N-(3-(S-methylsulfonimidoyl)phenyl)nicotinamide: A solution of tert-butyl (R)-((3-(2-(4,4-difluoroazepan-1-yl)-5-(3,4-difluorophenyl)-4-methylnicotinamido)phenyl)(methyl)(oxo)-λ⁶-sulfaneylidene)carbamate (86 mg, 0.136 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.05% NH₃) to provide (R)-2-(4,4-difluoroazepan-1-yl)-5-(3,4-difluorophenyl)-4-methyl-N-(3-(S-methylsulfonimidoyl)phenyl)nicotinamide (21.1 mg, 29%) as a white solid. ¹H NMR (400 MHz, DMSO-d₆) δ 10.87 (s, 1H), 8.37 (s, 1H), 8.10 (s, 1H), 7.90 (d, J=8.1 Hz, 1H), 7.67 (d, J=7.9 Hz, 1H), 7.59 (t, J=7.9 Hz, 1H), 7.56-7.49 (m, 1H), 7.49-7.43 (m, 1H), 7.21-7.17 (m, 1H), 4.22 (s, 1H), 3.68-3.64 (m, 2H), 3.59 (t, J=6.1 Hz, 2H), 3.06 (s, 3H), 2.37-2.22 (m, 2H), 2.16 (s, 3H), 2.07-1.94 (m, 2H), 1.85-1.79 (m, 2H). LCMS (ESI) calcd. for C₂₆H₂₇F₄N₄O₂S [M+H]⁺ m/z 535.18, found 535.15

### Example 60 (R)-2-(4,4-difluoroazepan-1-yl)-5-(3,4-difluorophenyl)-4-methyl-N-(3-(S-methyl-N-(methylglycyl)sulfonimidoyl phenyl)nicotinamide

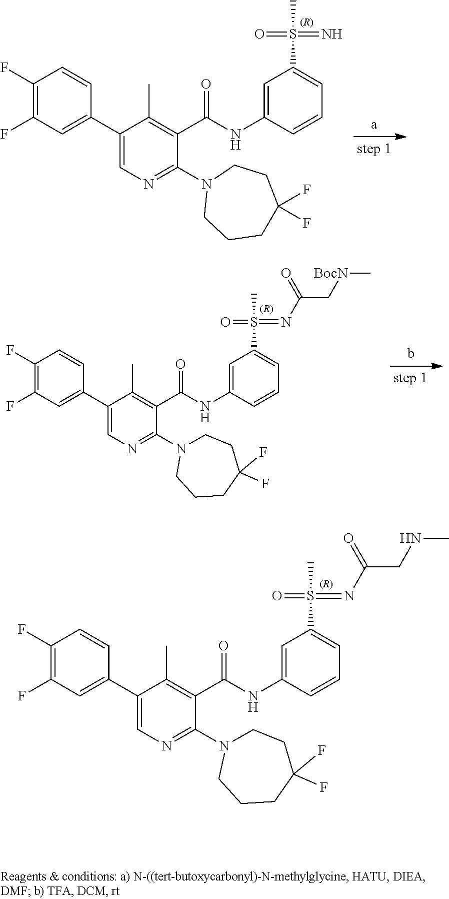

Step 1: tert-butyl (R)-(2-(((3-(2-(4,4-difluoroazepan-1-yl)-5-(3,4-difluorophenyl)-4-methylnicotinamido)phenyl)(methyl)(oxo)-λ⁶-sulfaneylidene)amino)-2-oxoethyl)(methyl)carbamate: A mixture of (R)-2-(4,4-difluoroazepan-1-yl)-5-(3,4-difluorophenyl)-4-methyl-N-(3-(S-methylsulfonimidoyl)phenyl)nicotinamide (140 mg, 0.26 mmol), N-(tert-butoxycarbonyl)-N-methylglycine (99.1 mg, 0.52 mmol) and HATU (149.4 mg, 0.39 mmol) in DMF (3 mL) was added DIEA (101.6 mg, 0.79 mmol) at room temperature. The reaction mixture was stirred at room temperature for 4 hours. After the reaction was completed, the resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtAOc=1/1) to give tert-butyl (R)-(2-(((3-(2-(4,4-difluoroazepan-1-yl)-5-(3,4-difluorophenyl)-4-methylnicotinamido)phenyl)(methyl)(oxo)-λ⁶-sulfaneylidene)amino)-2-oxoethyl)(methyl)carbamate (120 mg, 55.5%) as a yellow solid. LCMS (ESI) calcd. for C₃₄H₄₀F4N₅O₅S [M+H]⁺ m/z 706.27, found 706.25.

Step 2: (R)-2-(4,4-difluoroazepan-1-yl)-5-(3,4-difluorophenyl)-4-methyl-N-(3-(S-methyl-N-(methylglycyl)sulfonimidoyl)phenyl)nicotinamide: A solution of tert-butyl (R)-(2-(((3-(2-(4,4-difluoroazepan-1-yl)-5-(3,4-difluorophenyl)-4-methylnicotinamido)phenyl)(methyl)(oxo)-λ⁶-sulfaneylidene)amino)-2-oxoethyl)(methyl)carbamate (120 mg, 0.17 mmol) in DCM (3 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.05% NH₃) to provide (R)-2-(4,4-difluoroazepan-1-yl)-5-(3,4-difluorophenyl)-4-methyl-N-(3-(S-methyl-N-(methylglycyl)sulfonimidoyl)phenyl)nicotinamide (56.2 mg, 64.6%) as a white solid. ¹H NMR (400 MHz, DMSO-d₆) δ 10.97 (s, 1H), 8.39 (s, 1H), 8.11 (s, 1H), 7.99 (dd, J=6.8, 2.1 Hz, 1H), 7.70-7.63 (m, 2H), 7.56-7.42 (m, 2H), 7.23-7.16 (m, 1H), 3.70-3.64 (m, 2H), 3.58 (t, J=6.0 Hz, 2H), 3.46 (s, 3H), 3.20 (s, 2H), 2.39-2.7 (m, 2H), 2.24 (s, 3H), 2.17 (s, 3H), 2.08-1.95 (m, 2H), 1.87-1.77 (m, 2H). LCMS (ESI) calcd. for C₂₉H₃₂F₄N₅O₃S [M+H]⁺ m/z 606.22, found 606.15.

### Example 61

### (R)-2-(4,4-difluoroazepan-1-yl)-5-(3,5-difluorophenyl)-4-methyl-N-(3-(S-methylsulfonimidoyl)phenyl)nicotinamide

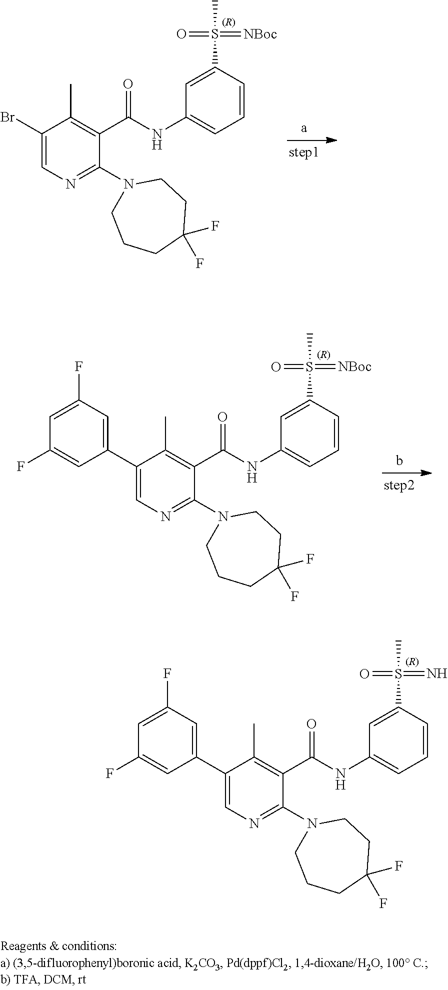

Step 1: tert-butyl (R)-((3-(2-(4,4-difluoroazepan-1-yl)-5-(3,5-difluorophenyl)-4-methylnicotinamido)phenyl)(methyl)(oxo)-λ⁶-sulfaneylidene)carbamate: A mixture of tert-butyl (R)-((3-(5-bromo-2-(4,4-difluoroazepan-1-yl)-4-methylnicotinamido)phenyl)(methyl)(oxo)-λ⁶-sulfaneylidene)carbamate(100 mg, 0.17 mmol), (3,5-difluorophenyl)boronic acid (53 mg, 0.33 mmol), K₂CO₃, (69 mg, 0.5 mmol) and Pd(dppf)Cl₂ (24 mg, 0.03 mmol) in 1,4-dioxane/H₂O (4/1, 5 mL) was heated at 100° C. for 5 h under an atmosphere of N₂. After the reaction was completed, the mixture was cooled to room temperature. The resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtAOc=1/1) to give tert-butyl (R)-((3-(2-(4,4-difluoroazepan-1-yl)-5-(3,5-difluorophenyl)-4-methylnicotinamido)phenyl)(methyl)(oxo)-λ⁶-sulfaneylidene)carbamate (70 mg, 64.8%) as a yellow oil. LCMS (ESI) calcd. for C₃₁H₃₅F₄N₄O₄S [M+H]⁺ m/z 635.23, found 635.20.

Step 2: (R)-2-(4,4-difluoroazepan-1-yl)-5-(3,5-difluorophenyl)-4-methyl-N-(3-(S-methylsulfonimidoyl)phenyl)nicotinamide: A solution of give tert-butyl (R)-((3-(2-(4,4-difluoroazepan-1-yl)-5-(3,5-difluorophenyl)-4-methylnicotinamido)phenyl)(methyl)(oxo)-λ⁶-sulfaneylidene)carbamate (70 mg, 0.11 mmol) in DCM (5 mL) was added TFA (1 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.05% NH₃) to provide (R)-2-(4,4-difluoroazepan-1-yl)-5-(3,5-difluorophenyl)-4-methyl-N-(3-(S-methylsulfonimidoyl)phenyl)nicotinamide (33.2 mg, 56.4%) as a white solid. ¹H NMR (400 MHz, DMSO-d₆) δ 10.87 (s, 1H), 8.36 (d, J=1.8 Hz, 1H), 8.12 (s, 1H), 7.90 (d, J=9.0 Hz, 1H), 7.70-7.53 (dt, J=15.8, 8.0 Hz, 2H), 7.33-7.19 (m, 1H), 7.15-7.04 (m, 2H), 4.22 (s, 1H), 3.70-3.55 (m, 4H), 3.06 (d, J=0.7 Hz, 3H), 2.32-2.23 (m, 2H), 2.18 (s, 3H), 2.07-1.95 (m, 2H), 1.88-1.76 (m, 2H). LCMS (ESI) calcd. for C₂₆H₂₇F₄N₄O₂S [M+H]⁺ m/z 535.18, found 535.10

### Example 62

### (R)-2-(4,4-difluoroazepan-1-yl)-5-(3,5-difluorophenyl)-4-methyl-N-(3-(S-methyl-N-(methylglycyl)sulfonimidoyl)phenyl)nicotinamide

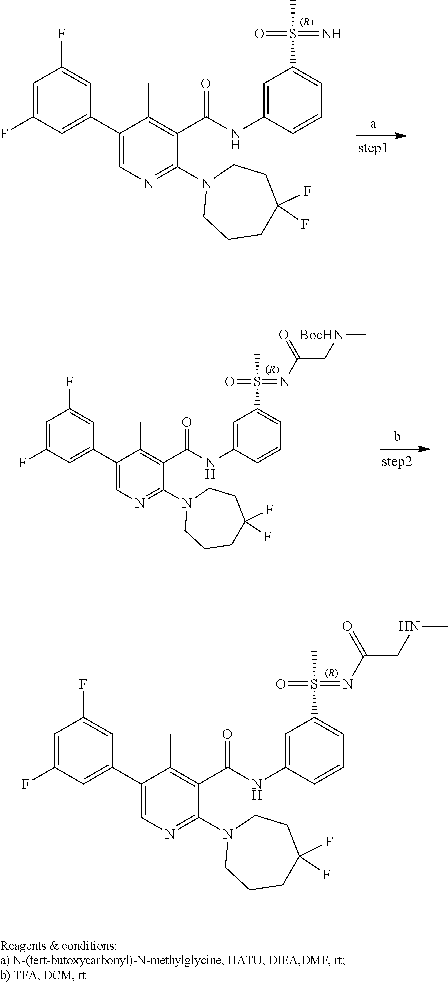

Step 1: tert-butyl (R)-(2-(((3-(2-(4,4-difluoroazepan-1-yl)-5-(3,5-difluorophenyl)-4-methylnicotinamido)phenyl)(methyl)(oxo)-λ⁶-sulfaneylidene)amino)-2-oxoethyl)(methyl)carbamate: A mixture of (R)-2-(4,4-difluoroazepan-1-yl)-5-(3,5-difluorophenyl)-4-methyl-N-(3-(S-methylsulfonimidoyl)phenyl)nicotinamide (130 mg, 0.24 mmol) and N-(tert-butoxycarbonyl)-N-methylglycine (55 mg, 0.29 mmol) in DMF (5 mL) was added HATU (137 mg, 0.36 mmol) and DIEA (93 mg, 0.72 mmol). The mixture was stirred at room temperature for 4 hours. The reaction was monitored by LCMS. After the reaction was completed, the resulting solution was diluted with water (50 mL) and extracted with DCM (20 mL×3). The combined organic phases were washed with brine, dried over Na₂SO₄, concentrated under vacuum. The residue was purified by flash column chromatography on silica gel (PE/EtOAc=2/1 to 1/1) to give tert-butyl (R)-(2-(((3-(2-(4,4-difluoroazepan-1-yl)-5-(3,5-difluorophenyl)-4-methylnicotinamido)phenyl)(methyl)(oxo)-λ⁶-sulfaneylidene)amino)-2-oxoethyl)(methyl)carbamate (105 mg, 61.9%) as a yellow solid. LCMS (ESI) calcd. for C₃₄H₄₀F4N₅O₅S [M+H]⁺ m/z 706.27, found 706.20.

Step 2: (R)-2-(4,4-difluoroazepan-1-yl)-5-(3,5-difluorophenyl)-4-methyl-N-(3-(S-methyl-N-(methylglycyl)sulfonimidoyl)phenyl)nicotinamide: A solution of tert-butyl (R)-(2-(((3-(2-(4,4-difluoroazepan-1-yl)-5-(3,5-difluorophenyl)-4-methylnicotinamido)phenyl)(methyl)(oxo)-λ⁶-sulfaneylidene)amino)-2-oxoethyl)(methyl)carbamate (105 mg, 0.15 mmol) in DCM (5 mL) was added TFA (0.5 mL) at room temperature. The reaction mixture was stirred at room temperature for 2 hours. After the reaction was completed, the solution was diluted with water (10 mL) and extracted with DCM (20 mL×3). The combined organic phases were washed with brine, dried over sodium sulfate, and concentrated under vacuum. The residue was purified by prep-HPLC (Gemini 5 um C₁₈ column, 150*21.2 mm, eluting with 50% to 95% MeCN/H₂O containing 0.05% NH₃) to obtain (R)-2-(4,4-difluoroazepan-1-yl)-5-(3,5-difluorophenyl)-4-methyl-N-(3-(S-methyl-N-(methylglycyl)sulfonimidoyl)phenyl)nicotinamide (53.5 mg, 58.8%) as a white solid. ¹H NMR (400 MHz, DMSO-d₆) δ 10.97 (s, 1H), 8.39 (d, J=10.8 Hz, 1H), 8.13 (s, 1H), 8.02-7.93 (m, 1H), 7.71-7.62 (m, 2H), 7.27 (t, J=9.5 Hz, 1H), 7.11 (d, J=6.8 Hz, 2H), 3.69-3.56 (m, 4H), 3.45 (s, 3H), 3.19 (s, 2H), 2.35-2.18 (m, 8H), 2.07-1.95 (m, 2H), 1.86-1.78 (m, 2H). LCMS (ESI) calcd. for C₂₉H₃₂F₄N₅O₃S [M+H]⁺ m/z 606.22, found 606.15.

### Example 63

### (R)-5-(cyclopropylethynyl)-2-(4,4-difluoroazepan-1-yl)-4-methyl-N-(3-(S-methylsulfonimidoyl)phenyl)nicotinamide

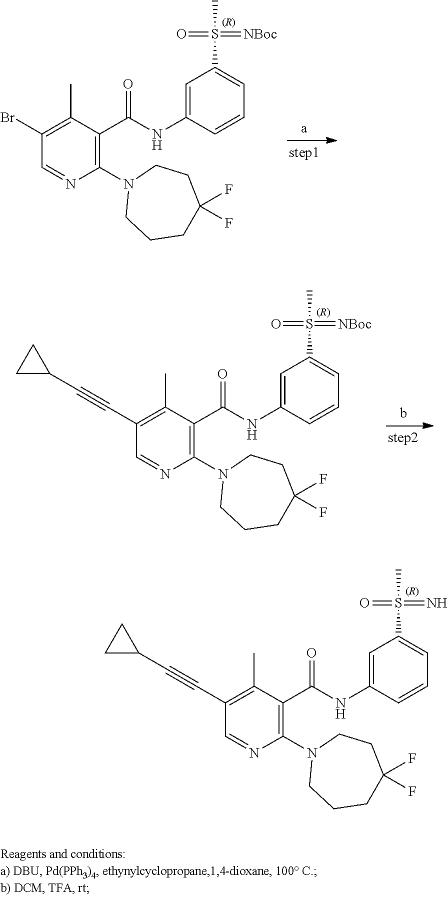

Step 1: tert-butyl (R)-((3-(5-(cyclopropylethynyl)-2-(4,4-difluoroazepan-1-yl)-4-methylnicotinamido) phenyl)(methyl)(oxo)-λ⁶-sulfaneylidene)carbamate: A mixture of tert-butyl (R)-((3-(5-bromo-2-(4,4-difluoroazepan-1-yl)-4-methylnicotinamido)phenyl)(methyl)(oxo)-λ⁶-sulfaneylidene)carbamate (200 mg, 0.33 mmol), ethynylcyclopropane (66 mg, 0.80 mmol), DBU (150 mg, 0.99 mmol) and Pd(PPh₃)₄ (20 mg, 0.03 mmol) in 1,4-dioxane (4 mL) was heated at 100° C. for 16 h under an atmosphere of N₂.

After the reaction was completed, the mixture was cooled to room temperature. The resulting solution was diluted with water (40 mL) and extracted with EtOAc (30 mL×3). The combined organic layers were washed with brine, dried over Na₂SO₄, concentrated under vacuum. The residue was purified by flash column chromatography on silica gel (PE/EtOAc=3/1) to give tert-butyl (R)-((3-(5-(cyclopropylethynyl)-2-(4,4-difluoroazepan-1-yl)-4-methylnicotinamido)phenyl)(methyl)(oxo)-λ⁶-sulfaneylidene)carbamate (210 mg, 81.6%) as a yellow solid. LCMS (ESI) calcd. for C₃₀H₃₇F₂N₄O₄S [M+H]⁺ m/z 587.25, found 587.25.

Step 2: (R)-5-(cyclopropylethynyl)-2-(4,4-difluoroazepan-1-yl)-4-methyl-N-(3-(S-methylsulfonimidoyl)phenyl)nicotinamide: A solution of tert-butyl (R)-((3-(5-(cyclopropylethynyl)-2-(4,4-difluoroazepan-1-yl)-4-methylnicotinamido)phenyl)(methyl)(oxo)-λ⁶-sulfaneylidene)carbamate (210 mg, 0.35 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The combined organic phases were washed with brine, dried with Na₂SO₄, and concentrated under reduced pressure to afford (R)-5-(cyclopropylethynyl)-2-(4,4-difluoroazepan-1-yl)-4-methyl-N-(3-(S-methylsulfonimidoyl)phenyl)nicotinamide (200 mg, crude) as a yellow solid. LCMS (ESI) calcd. for C₂₅H₂₉F₂N₄O₂S [M+H]⁺ m/z 487.20, found 487.10.

### Example 64

### (R)-5-(cyclopropylethynyl)-2-(4,4-difluoroazepan-1-yl)-4-methyl-N-(3-(S-methyl-N-(methylglycyl)sulfonimidoyl)phenyl)nicotinamide

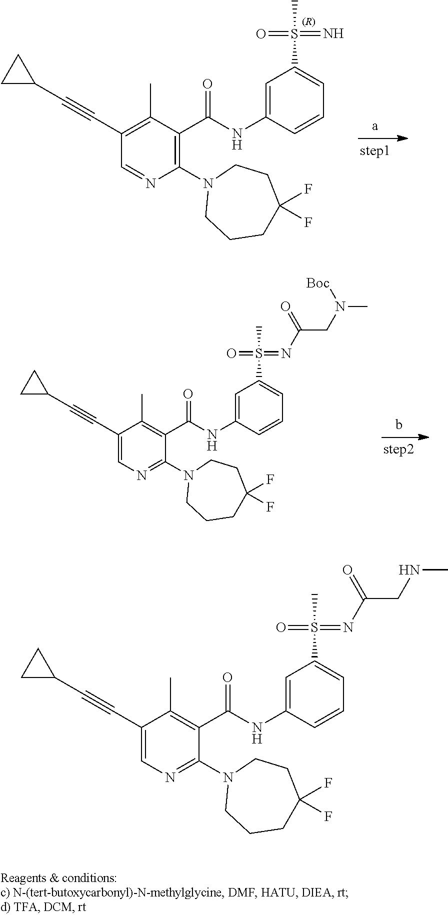

Step 1: tert-butyl (R)-(2-(((3-(5-(cyclopropylethynyl)-2-(4,4-difluoroazepan-1-yl)-4-methylnicotinamido)phenyl)(methyl)(oxo)-λ⁶-sulfaneylidene)amino)-2-oxoethyl) (methyl) carbamate: A solution of (R)-5-(cyclopropylethynyl)-2-(4,4-difluoroazepan-1-yl)-4-methyl-N-(3-(S-methylsulfonimidoyl)phenyl)nicotinamide (200 mg, 0.41 mmol) in DMF (2 mL) was added N-(tert-butoxycarbonyl)-N-methylglycine (117 mg, 0.66 mmol), DIEA (169 mg, 1.23 mmol) and HATU (277 mg, 0.82 mmol) at 0° C. The resulting mixture was stirred at 25° C. for 1 hour. Then the mixture was quenched with water (20 mL) and extracted with EtOAc (30 mL×2). The combined organic layers were washed with brine, dried over sodium sulfate, concentrated under vacuum. The residue was purified by flash column chromatography on silica gel (PE/EtOAc=1/1) to afford tert-butyl (R)-(2-(((3-(5-(cyclopropylethynyl)-2-(4,4-difluoroazepan-1-yl)-4-methylnicotinamido)phenyl)(methyl)(oxo)-λ⁶-sulfaneylidene)amino)-2-oxoethyl)(methyl)carbamate (120 mg, 38.4%) as a yellow solid. LCMS (ESI) calcd. for C₃₃H₄₂F₂N₅O₅S [M+H]⁺ m/z 658.29, found 658.25.

Step 2: (R)-5-(cyclopropylethynyl)-2-(4,4-difluoroazepan-1-yl)-4-methyl-N-(3-(S-methyl-N-(methylglycyl)sulfonimidoyl)phenyl)nicotinamide: A solution of tert-butyl (R)-(2-(((3-(5-(cyclopropylethynyl)-2-(4,4-difluoroazepan-1-yl)-4-methylnicotinamido)phenyl)(methyl)(oxo)-λ⁶-sulfaneylidene)amino)-2-oxoethyl)(methyl)carbamate (120 mg, 0.18 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 30% to 90% MeCN/H₂O containing 0.05% NH₃) to provide (R)-5-(cyclopropylethynyl)-2-(4,4-difluoroazepan-1-yl)-4-methyl-N-(3-(S-methyl-N-(methylglycyl)sulfonimidoyl)phenyl)nicotinamide (35.5 mg, 37.5%) as a white solid. ¹H NMR (400 MHz, DMSO-d₆) δ 10.87 (s, 1H), 8.35 (s, 1H), 8.17 (s, 1H), 7.96 (d, J=7.3 Hz, 1H), 7.67 (t, J=7.2 Hz, 2H), 3.66-3.59 (m, 2H), 3.56-3.51 (m, 2H), 3.46 (s, 3H), 3.19 (s, 2H), 2.29-2.19 (m, 8H), 2.03-1.94 (m, 2H), 1.83-1.76 (m, 2H), 1.61-1.53 (m, 1H), 0.92-0.87 (m, 2H), 0.74-0.69 (m, 2H). LCMS (ESI) calcd. for C₂₈H₃₄F₂N₅O₃S [M+H]⁺ m/z 558.23, found 558.20.

### Example 65

### (R)-5-cyano-2-(4,4-difluoroazepan-1-yl)-4-methyl-N-(3-(S-methyl-N-(methylglycyl)sulfonimidoyl)phenyl)-6-(trifluoromethyl)nicotinamide

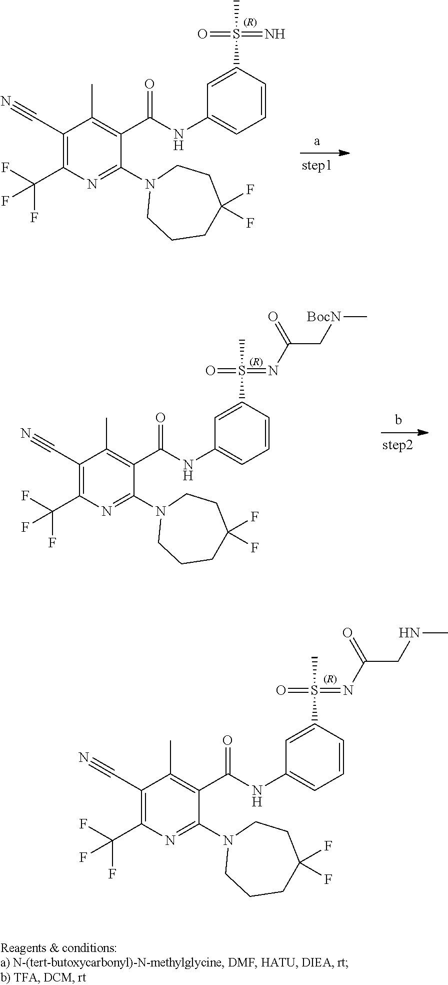

Step 1: tert-butyl (R)-(2-(((3-(5-cyano-2-(4,4-difluoroazepan-1-yl)-4-methyl-6-(trifluoromethyl)nicotinamido)phenyl)(methyl)(oxo)-λ⁶-sulfaneylidene)amino)-2-oxoethyl)(methyl)carbamate: A mixture of (R)-5-cyano-2-(4,4-difluoroazepan-1-yl)-4-methyl-N-(3-(S-methylsulfonimidoyl)phenyl)-6-(trifluoromethyl)nicotinamide (100 mg, 0.19 mmol) and N-(tert-butoxycarbonyl)-N-methylglycine (74 mg, 0.39 mmol) in DMF (10 mL) was added HATU (96 mg, 0.252 mmol) and DIEA (80 mg, 0.621 mmol). The mixture was stirred at room temperature for 4 hours. The reaction was monitored by LCMS. After the reaction was completed, the resulting solution was diluted with water (50 mL) and extracted with DCM (20 mL×3). The combined organic phases were washed with brine, dried over Na₂SO₄, concentrated under vacuum. The residue was purified by flash column chromatography on silica gel (PE/EtOAc=2/1 to 1/1) to give tert-butyl (R)-(2-(((3-(5-cyano-2-(4,4-difluoroazepan-1-yl)-4-methyl-6-(trifluoromethyl)nicotinamido)phenyl)(methyl)(oxo)-λ⁶-sulfaneylidene)amino)-2-oxoethyl)(methyl)carbamate (105 mg, 78.9%) as a white oil. LCMS (ESI) calcd. for C₃₀H₃₄F₅N₆O₅S [M−H]⁺ m/z 685.13, found 685.10.

Step 2: (R)-5-cyano-2-(4,4-difluoroazepan-1-yl)-4-methyl-N-(3-(S-methyl-N-(methylglycyl)sulfonimidoyl)phenyl)-6-(trifluoromethyl)nicotinamide: A solution of tert-butyl (R)-(2-(((3-(5-cyano-2-(4,4-difluoroazepan-1-yl)-4-methyl-6-(trifluoromethyl)nicotinamido)phenyl)(methyl)(oxo)-λ⁶-sulfaneylidene)amino)-2-oxoethyl)(methyl)carbamate (105 mg, 0.15 mmol) in DCM (5 mL) was added TFA (0.5 mL) at room temperature. The reaction mixture was stirred at room temperature for 2 hours. After the reaction was completed, the solution was diluted with water (10 mL) and extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.1% formic acid) to obtain (R)-5-cyano-2-(4,4-difluoroazepan-1-yl)-4-methyl-N-(3-(S-methyl-N-(methylglycyl)sulfonimidoyl)phenyl)-6-(trifluoromethyl)nicotinamide (46.1 mg, 51.79%) as a white solid. ¹H NMR (400 MHz, CD₃OD) δ 8.34 (s, 1H), 7.86 (d, J=8.4 Hz, 1H), 7.71 (d, J=8 Hz, 1H), 7.60-7.56 (m, 1H), 3.78-3.75 (m, 2H), 3.66-3.63 (m, 2H), 3.34 (s, 3H), 3.26 (s, 2H), 2.45 (s, 3H), 2.26 (s, 3H), 2.26-2.21 (m, 2H), 1.96-1.89 (m, 2H), 1.86-1.83 (m, 2H). LCMS (ESI) calcd. for C₂₅H₂₈F₅N₆O₃S [M+H]⁺ m/z 587.28, found 587.15.

### Example 66

### (R)-N-(3-(N-(1-aminocyclopropane-1-carbonyl)-S-methylsulfonimidoyl)phenyl)-5-cyano-2-(4,4-difluoroazepan-1-yl)-4-methyl-6-(trifluoromethyl)nicotinamide

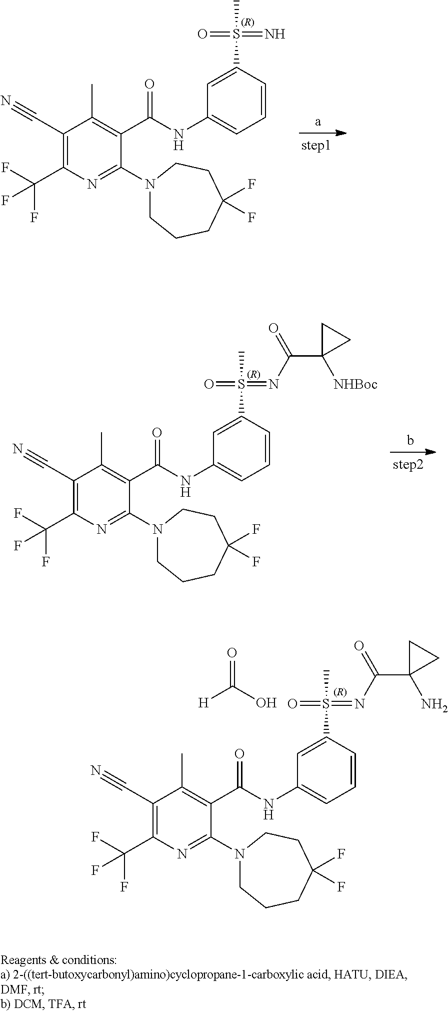

Step 1: tert-butyl (R)-(1-(((3-(5-cyano-2-(4,4-difluoroazepan-1-yl)-4-methyl-6-(trifluoromethyl)nicotinamido)phenyl)(methyl)(oxo)-λ⁶-sulfaneylidene)carbamoyl)cyclopropyl)carbamate: A mixture of (R)-5-cyano-2-(4,4-difluoroazepan-1-yl)-4-methyl-N-(3-(S-methylsulfonimidoyl)phenyl)-6-(trifluoromethyl)nicotinamide (110 mg, 0.21 mmol) and 1-((tert-butoxycarbonyl)amino)cyclopropane-1-carboxylic acid (37 mg, 0.32 mmol) in DMF (10 mL) was added HATU (162 mg, 0.43 mmol) and DIEA (82 mg, 0.64 mmol). The mixture was stirred at room temperature for 4 hours. After the reaction was completed, the resulting solution was diluted with water (40 mL) and extracted with DCM (40 mL×3). The combined organic phases were washed with brine, dried over Na₂SO₄, concentrated under vacuum. The residue was purified by flash column chromatography on silica gel (PE/EtOAc=2/1 to 1/1) to give tert-butyl (R)-(1-(((3-(5-cyano-2-(4,4-difluoroazepan-1-yl)-4-methyl-6-(trifluoromethyl)nicotinamido)phenyl)(methyl)(oxo)-λ⁶-sulfaneylidene)carbamoyl)cyclopropyl)carbamate (105 mg, 80.32%) as a colorless oil. LCMS (ESI) calcd. for C₃₁H₃₆F₅N₆O₅S [M−H]⁺ m/z 699.24, found 699.10.

Step 2: (R)-N-(3-(N-(1-aminocyclopropane-1-carbonyl)-S-methylsulfonimidoyl)phenyl)-5-cyano-2-(4,4-difluoroazepan-1-yl)-4-methyl-6-(trifluoromethyl)nicotinamide: A solution of tert-butyl (R)-(1-(((3-(5-cyano-2-(4,4-difluoroazepan-1-yl)-4-methyl-6-(trifluoromethyl)nicotinamido)phenyl)(methyl)(oxo)-λ⁶-sulfaneylidene)carbamoyl)cyclopropyl)carbamate (105 mg, 0.15 mmol) in DCM (5 mL) was added TFA (0.5 mL) at room temperature. The reaction mixture was stirred at room temperature for 2 hours. After the reaction was completed, the solution was diluted with water (20 mL) and extracted with DCM (20 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.1% formic acid) to obtain (R)-N-(3-(N-(1-aminocyclopropane-1-carbonyl)-S-methylsulfonimidoyl)phenyl)-5-cyano-2-(4,4-difluoroazepan-1-yl)-4-methyl-6-(trifluoromethyl)nicotinamide formate (60.5 mg, 66.56%) as a white solid. ¹H NMR (400 MHz, DMSO-d₆) δ 11.17-11.04 (m, 1H), 8.31 (s, 1H), 8.23-8.16 (m, 1H), 7.94 (d, J=6.9 Hz, 1H), 7.70 (q, J=8.6, 7.9 Hz, 2H), 3.88-3.71 (m, 2H), 3.66 (t, J=5.5 Hz, 2H), 3.46 (s, 3H), 2.48 (s, 3H), 2.36-2.22 (m, 2H), 2.10-1.96 (m, 2H), 1.91-1.80 (m, 2H), 1.28-1.18 (m, 2H), 0.92-0.79 (m, 2H). LCMS (ESI) calcd. for C₂₆H₂₈F₅N₆O₃S [M+H]⁺ m/z 599.19, found 599.20.

### Example 67

### (R)-5-(4-chlorophenyl)-2-(4,4-difluoroazepan-1-yl)-4-methyl-N-(3-(S-methylsulfonimidoyl)phenyl)nicotinamide

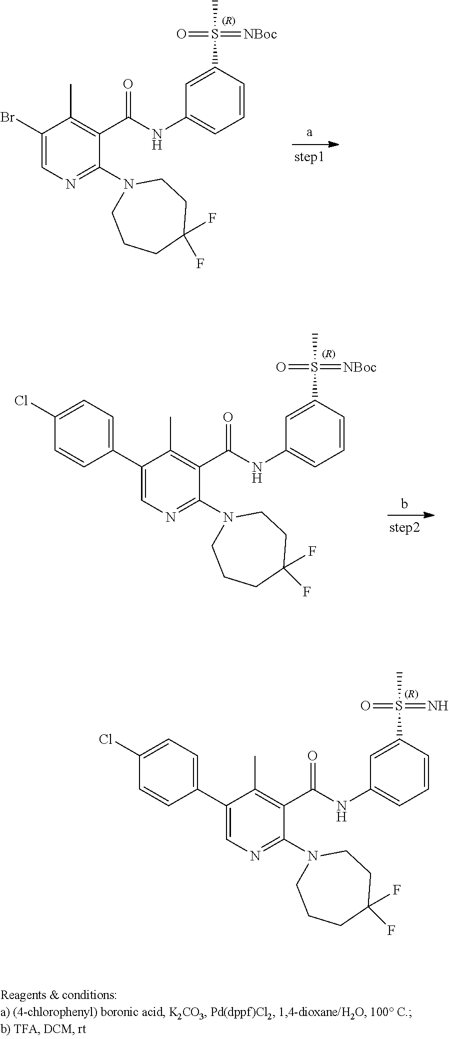

Step 1: tert-butyl (R)-((3-(5-(4-chlorophenyl)-2-(4,4-difluoroazepan-1-yl)-4-methylnicotinamido)phenyl)(methyl)(oxo)-λ⁶-sulfaneylidene)carbamate: A solution of tert-butyl (R)-((3-(5-bromo-2-(4,4-difluoroazepan-1-yl)-4-methylnicotinamido)phenyl)(methyl)(oxo)-λ⁶-sulfaneylidene)carbamate (100 mg, 0.17 mmol), (4-chlorophenyl) boronic acid (32 mg, 0.20 mmol), K₂CO₃ (46 mg, 0.33 mmol) and Pd(dppf)Cl₂ (12 mg, 0.02 mmol) in dioxane (10 mL) and H₂O (2.5 mL) was heated at 100° C. for 6 hours. LCMS showed the reaction was completed. Then the mixture was diluted with water (10 mL) and extracted with EtOAc (10 mL×3). The combined organic layers were washed with brine, dried over sodium sulfate, concentrated under vacuum. The residue was purified by flash column chromatography on silica gel (PE/EtOAc=1/1) to provide tert-butyl (R)-((3-(5-(4-chlorophenyl)-2-(4,4-difluoroazepan-1-yl)-4-methylnicotinamido)phenyl)(methyl)(oxo)-λ⁶-sulfaneylidene)carbamate (90 mg, 85.71%) as yellow oil. LCMS (ESI) calcd. for C₃₁H₃₆ClF₂N₄O₄S [M+H]⁺ m/z 633.21, found 633.10.

Step 2: (R)-5-(4-chlorophenyl)-2-(4,4-difluoroazepan-1-yl)-4-methyl-N-(3-(S-methylsulfonimidoyl)phenyl)nicotinamide: A solution of tert-butyl (R)-((3-(5-(4-chlorophenyl)-2-(4,4-difluoroazepan-1-yl)-4-methylnicotinamido)phenyl)(methyl)(oxo)-λ⁶-sulfaneylidene)carbamate (90 mg, 0.14 mmol) in DCM (3 mL) was added TFA (0.3 mL) at room temperature. The reaction mixture was stirred at room temperature for 1 hour. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.05% NH₃·H₂O) to provide (R)-5-(4-chlorophenyl)-2-(4,4-difluoroazepan-1-yl)-4-methyl-N-(3-(S-methylsulfonimidoyl)phenyl)nicotinamide (45.07 mg, 58.27%) as a white solid. ¹H NMR (400 MHz, DMSO-d₆) δ 10.88 (s, 1H), 8.37 (s, 1H), 8.08 (s, 1H), 7.90 (d, J=8.1 Hz, 1H), 7.67 (d, J=7.7 Hz, 1H), 7.58 (t, J=7.8 Hz, 1H), 7.52 (d, J=8.3 Hz, 2H), 7.37 (d, J=8.4 Hz, 2H), 4.22 (s, 1H), 3.70-3.62 (m, 2H), 3.61-3.54 (m, 2H), 3.06 (s, 3H), 2.31-2.23 (m, 2H), 2.15 (s, 3H), 2.06-1.96 (m, 2H), 1.85-1.76 (m, 2H). LCMS (ESI) calcd. for C₂₆H₂₈ClF₂N₄O₂S [M+H]⁺ m/z 533.16, found 533.15.

### Example 68

### (R)-5-(4-chlorophenyl)-2-(4,4-difluoroazepan-1-yl)-4-methyl-N-(3-(S-methyl-N-(methylglycyl)sulfonimidoyl)phenyl)nicotinamide

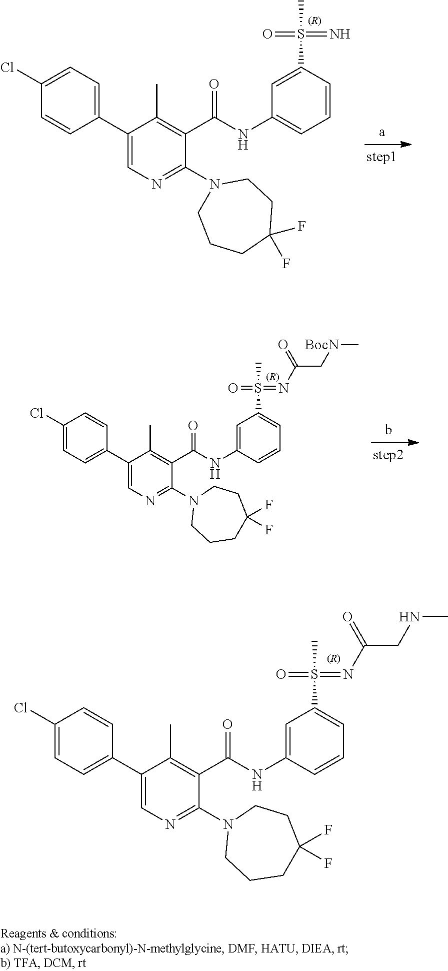

Step 1: tert-butyl (R)-(2-(((3-(5-(4-chlorophenyl)-2-(4,4-difluoroazepan-1-yl)-4-methylnicotinamido)phenyl)(methyl)(oxo)-λ⁶-sulfaneylidene)amino)-2-oxoethyl)(methyl)carbamate: A solution of (R)-5-(4-chlorophenyl)-2-(4,4-difluoroazepan-1-yl)-4-methyl-N-(3-(S-methylsulfonimidoyl)phenyl)nicotinamide (110 mg, 0.21 mmol), N-(tert-butoxycarbonyl)-N-methylglycine (78 mg, 0.41 mmol), HATU (157 mg, 0.41 mmol) and DIEA (80 mg, 0.62 mmol) in DMF (5 mL) was stirred at room temperature for 16 hours. LCMS showed the reaction was completed. The mixture was diluted with water (40 mL) and extracted with EtOAc (20 mL×3). The combined organic layers were washed with brine, dried over sodium sulfate, concentrated under vacuum. The residue was purified by flash column chromatography on silica gel (PE/EtOAc=1/1) to provide tert-butyl (R)-(2-(((3-(5-(4-chlorophenyl)-2-(4,4-difluoroazepan-1-yl)-4-methylnicotinamido)phenyl)(methyl)(oxo)-λ⁶-sulfaneylidene)amino)-2-oxoethyl)(methyl)carbamate (110 mg, 75.86%) as yellow oil. LCMS (ESI) calcd. for C₃₄H₄₁ClF₂N₅O₅S [M+H]⁺ m/z 704.25, found 704.20.

Step 2: (R)-5-(4-chlorophenyl)-2-(4,4-difluoroazepan-1-yl)-4-methyl-N-(3-(S-methyl-N-(methylglycyl)sulfonimidoyl)phenyl)nicotinamide: A solution of tert-butyl (R)-(2-(((3-(5-(4-chlorophenyl)-2-(4,4-difluoroazepan-1-yl)-4-methylnicotinamido)phenyl)(methyl)(oxo)-λ⁶-sulfaneylidene)amino)-2-oxoethyl)(methyl)carbamate (110 mg, 0.16 mmol) in DCM (2 mL) was added TFA (0.2 mL) at room temperature. The reaction mixture was stirred at room temperature for 1 hour. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 30% to 95% MeCN/H₂O containing 0.1% formic acid) to provide (R)-5-(4-chlorophenyl)-2-(4,4-difluoroazepan-1-yl)-4-methyl-N-(3-(S-methyl-N-(methylglycyl)sulfonimidoyl)phenyl)nicotinamide formate (36.88 mg, 98.7%) as a white solid. ¹H NMR (400 MHz, DMSO-d₆) δ 11.12 (s, 1H), 8.39 (d, J=9.4 Hz, 2H), 8.08 (s, 1H), 8.01 (d, J=7.2 Hz, 1H), 7.71-7.63 (m, 2H), 7.52 (d, J=8.3 Hz, 2H), 7.37 (d, J=8.3 Hz, 2H), 3.66 (s, 2H), 3.58 (t, J=5.7 Hz, 2H), 3.47 (s, 3H), 3.28 (s, 2H), 2.36-2.21 (s, 5H), 2.15 (s, 3H), 2.06-1.95 (m, 2H), 1.86-1.76 (m, 2H). LCMS (ESI) calcd. for C₂₉H₃₃ClF₂N₅O₃S [M+H]⁺ m/z 604.20, found 604.15.

### Example 69

### (R)-5-(1-cyclopropyl-1H-pyrazol-4-yl)-2-(4,4-difluoroazepan-1-yl)-4-methyl-N-(3-(S-methylsulfonimidoyl)phenyl)nicotinamide

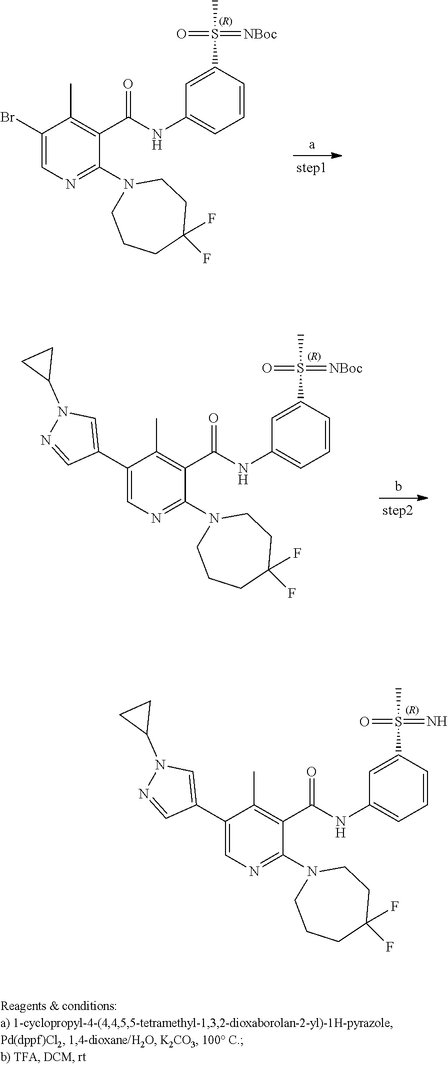

Step 1: tert-butyl (R)-((3-(5-(1-cyclopropyl-1H-pyrazol-4-yl)-2-(4,4-difluoroazepan-1-yl)-4-methylnicotinamido)phenyl)(methyl)(oxo)-λ⁶-sulfaneylidene)carbamate: A mixture of tert-butyl (R)-((3-(5-bromo-2-(4,4-difluoroazepan-1-yl)-4-methylnicotinamido)phenyl)(methyl)(oxo)-λ⁶-sulfaneylidene)carbamate (300 mg, 0.49 mmol), 1-cyclopropyl-4-(4,4,5,5-tetramethyl-1,3,2-dioxaborolan-2-yl)-1H-pyrazole (234 mg, 0.99 mmol), K₂CO₃ (207 mg, 1.49 mmol) and [1,1′-Bis(diphenylphosphino)ferrocene]dichloropalladium (II) (37 mg, 0.05 mmol) in 1,4-dioxane/H₂O (4/1, 15 mL) was heated at 100° C. for 6 hours under an atmosphere of N₂. The reaction was monitored by LCMS. After the reaction was completed, the mixture was cooled to room temperature. The resulting solution was diluted with water (80 mL) and extracted with EA (40 mL×3). The combined organic layers were washed with brine, dried over Na₂SO₄, and concentrated under vacuum. The residue was purified by flash column chromatography on silica gel (PE/EtOAc=2/1 to 1/1) to give tert-butyl (R)-((3-(5-(1-cyclopropyl-1H-pyrazol-4-yl)-2-(4,4-difluoroazepan-1-yl)-4-methylnicotinamido)phenyl)(methyl)(oxo)-λ⁶-sulfaneylidene)carbamate (300 mg, 95.54%) as a yellow solid. LCMS (ESI) calcd. for C₃₁H₃₉F₂N₆O₄S [M+H]⁺ m/z 629.27, found 629.35.

Step 2: (R)-5-(1-cyclopropyl-1H-pyrazol-4-yl)-2-(4,4-difluoroazepan-1-yl)-4-methyl-N-(3-(S-methylsulfonimidoyl)phenyl)nicotinamide: A solution of tert-butyl (R)-((3-(5-(1-cyclopropyl-1H-pyrazol-4-yl)-2-(4,4-difluoroazepan-1-yl)-4-methylnicotinamido)phenyl)(methyl)(oxo)-λ⁶-sulfaneylidene)carbamate (100 mg, 0.16 mmol) in DCM (5 mL) was added TFA (0.5 mL) at room temperature. The reaction mixture was stirred at room temperature for 2 hours. After the reaction was completed, the solution was diluted with water (20 mL) and extracted with DCM (30 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.1% formic acid) to obtain (R)-5-(1-cyclopropyl-1H-pyrazol-4-yl)-2-(4,4-difluoroazepan-1-yl)-4-methyl-N-(3-(S-methylsulfonimidoyl)phenyl)nicotinamide (60.7 mg, 71.4%) as a white solid. ¹H NMR (400 MHz, DMSO-d₆) δ 10.85 (s, 1H), 8.37 (s, 1H), 8.17 (s, 1H), 7.98 (s, 1H), 7.90 (d, J=8.6 Hz, 1H), 7.66 (d, J=7.9 Hz, 1H), 7.62-7.53 (m, 2H), 4.22 (s, 1H), 3.79-3.74 (m, 1H), 3.64-3.51 (m, 4H), 3.06 (s, 3H), 2.34-2.20 (m, 5H), 2.05-1.92 (m, 2H), 1.86-1.72 (m, 2H), 1.13-1.05 (m, 2H), 1.03-0.94 (m, 2H). LCMS (ESI) calcd. for C₂₆H₃₁F₂N₆O₂S [M+H]⁺ m/z 529.22, found 529.20.

### Example 70

### (R)-5-(1-cyclopropyl-1H-pyrazol-4-yl)-2-(4,4-difluoroazepan-1-yl)-4-methyl-N-(3-(S-methyl-N-(methylglycyl)sulfonimidoyl)phenyl)nicotinamide

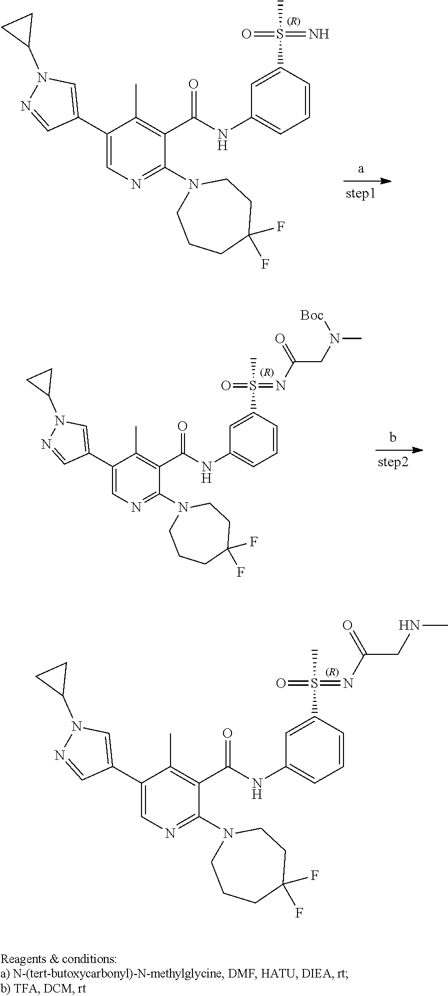

Step 1: tert-butyl (R)-(2-(((3-(5-(1-cyclopropyl-1H-pyrazol-4-yl)-2-(4,4-difluoroazepan-1-yl)-4-methylnicotinamido)phenyl)(methyl)(oxo)-λ⁶-sulfaneylidene)amino)-2-oxoethyl)(methyl)carbamate: A mixture of (R)-5-(1-cyclopropyl-1H-pyrazol-4-yl)-2-(4,4-difluoroazepan-1-yl)-4-methyl-N-(3-(S-methylsulfonimidoyl)phenyl)nicotinamide (140 mg, 0.27 mmol), N-(tert-butoxycarbonyl)-N-methylglycine (100 mg, 0.53 mmol) and HATU (201 mg, 0.53 mmol) in DMF (3 mL) was added DIEA (104 mg, 0.81 mmol) at room temperature. The reaction mixture was heated at 50° C. for 2 hours. After the reaction was completed, the resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtAOc=1/1) to give tert-butyl (R)-(2-(((3-(5-(1-cyclopropyl-1H-pyrazol-4-yl)-2-(4,4-difluoroazepan-1-yl)-4-methylnicotinamido)phenyl)(methyl)(oxo)-λ⁶-sulfaneylidene)amino)-2-oxoethyl)(methyl)carbamate (140 mg, 75.68%) as a yellow solid. LCMS (ESI) calcd. for C₃₄H₄₄F₂N₇O₅S [M+H]⁺ m/z 700.31, found 700.25.

Step 2: (R)-5-(1-cyclopropyl-1H-pyrazol-4-yl)-2-(4,4-difluoroazepan-1-yl)-4-methyl-N-(3-(S-methyl-N-(methylglycyl)sulfonimidoyl)phenyl)nicotinamide: A solution of tert-butyl (R)-(2-(((3-(5-(1-cyclopropyl-1H-pyrazol-4-yl)-2-(4,4-difluoroazepan-1-yl)-4-methylnicotinamido)phenyl)(methyl)(oxo)-λ⁶-sulfaneylidene)amino)-2-oxoethyl)(methyl)carbamate (140 mg, 0.20 mmol) in DCM (3 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.05% NH₃) to provide (R)-5-(1-cyclopropyl-1H-pyrazol-4-yl)-2-(4,4-difluoroazepan-1-yl)-4-methyl-N-(3-(S-methyl-N-(methylglycyl)sulfonimidoyl)phenyl)nicotinamide (82.5 mg, 68.75%) as a white solid. ¹H NMR (400 MHz, DMSO-d₆) δ 10.93 (s, 1H), 8.38 (s, 1H), 8.18 (s, 1H), 8.01-7.95 (m, 2H), 7.69-7.63 (m, 2H), 7.60 (d, J=0.5 Hz, 1H), 3.80-3.72 (m, 1H), 3.63-3.58 (m, 2H), 3.54 (t, J=6.1 Hz, 2H), 3.45 (s, 3H), 3.19 (s, 2H), 2.34-2.17 (m, 8H), 2.06-1.92 (m, 2H), 1.84-1.74 (m, 2H), 1.12-1.05 (m, 2H), 1.02-0.94 (m, 2H). LCMS (ESI) calcd. for C₂₉H₃₆F₂N₇O₃S [M+H]⁺ m/z 600.26, found 600.40.

### Example 71

### (R)-3-(4,4-difluoroazepan-1-yl)-5-methyl-6-(1-methyl-1H-pyrazol-4-yl)-N-(3-(S-methylsulfonimidoyl)phenyl)pyridazine-4-carboxamide

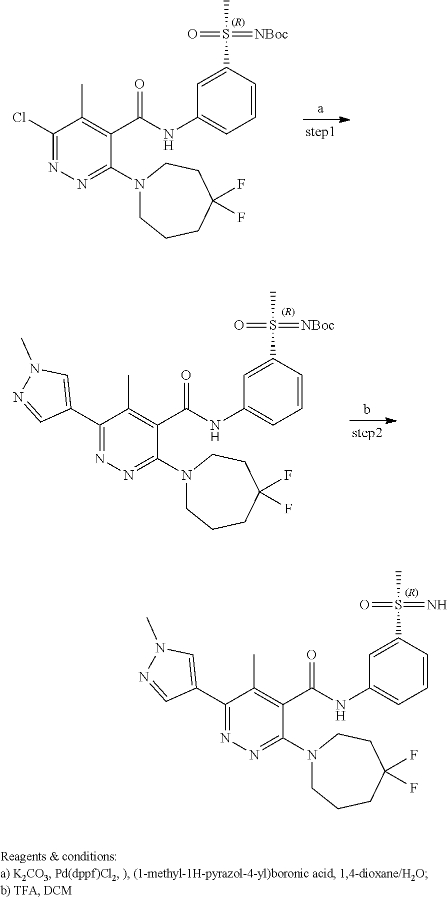

Step 1: tert-butyl (R)-((3-(3-(4,4-difluoroazepan-1-yl)-5-methyl-6-(1-methyl-1H-pyrazol-4-yl)pyridazine-4-carboxamido)phenyl)(methyl)(oxo)-λ⁶-sulfaneylidene)carbamate: A mixture of tert-butyl (R)-((3-(6-chloro-3-(4,4-difluoroazepan-1-yl)-5-methylpyridazine-4-carboxamido)phenyl)(methyl)(oxo)-λ⁶-sulfaneylidene)carbamate (300 mg, 0.54 mmol), (1-methyl-1H-pyrazol-4-yl)boronic acid (204 mg, 1.62 mmol), K₂CO₃ (224 mg, 1.62 mmol) and Pd(dppf)Cl₂ (36 mg, 0.05 mmol) in 1,4-dioxane (20 mL) and H₂O (5 mL) was heated at 100° C. for 16 hours under an atmosphere of N₂. After the reaction was completed, the mixture was cooled to room temperature. The resulting solution was diluted with water (100 mL) and extracted with EtOAc (40 mL×3). The combined organic phases were washed with brine, dried over Na₂SO₄, concentrated under vacuum. The residue was purified by flash column chromatography on silica gel (PE/EtOAc=1/1) to give tert-butyl (R)-((3-(3-(4,4-difluoroazepan-1-yl)-5-methyl-6-(1-methyl-1H-pyrazol-4-yl)pyridazine-4-carboxamido)phenyl)(methyl)(oxo)-λ⁶-sulfaneylidene)carbamate (290 mg, 89.2%) as a yellow solid. LCMS (ESI) calcd. for C₂₈H₃₆F₂N₇O₄S [M+H]⁺ m/z 604.25, found 604.15.

Step 2: (R)-3-(4,4-difluoroazepan-1-yl)-5-methyl-6-(1-methyl-1H-pyrazol-4-yl)-N-(3-(S-methylsulfonimidoyl)phenyl)pyridazine-4-carboxamide: A solution of tert-butyl (R)-((3-(3-(4,4-difluoroazepan-1-yl)-5-methyl-6-(1-methyl-1H-pyrazol-4-yl)pyridazine-4-carboxamido)phenyl)(methyl)(oxo)-λ⁶-sulfaneylidene)carbamate (290 mg, 0.48 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (30 mL×3). The combined organic phases were washed with brine, dried over sodium sulfate, concentrated under vacuum to give the crude product. Half of the crude product was purified by prep-HPLC (Gemini 5 um C₁₈ column, 150*21.2 mm, eluting with 40% to 90% MeCN/H₂O containing 0.1% NH₃) to provide (R)-3-(4,4-difluoroazepan-1-yl)-5-methyl-6-(1-methyl-1H-pyrazol-4-yl)-N-(3-(S-methylsulfonimidoyl)phenyl)pyridazine-4-carboxamide (15.1 mg, 12.4%) as a white solid. ¹H NMR (400 MHz, DMSO-d₆) δ 11.07 (s, 1H), 8.32 (s, 1H), 8.18 (s, 1H), 7.88 (d, J=7.5 Hz, 2H), 7.70 (d, J=8.0 Hz, 1H), 7.61 (t, J=7.9 Hz, 1H), 4.25 (s, 1H), 3.92 (s, 3H), 3.76-3.68 (m, 2H), 3.62 (t, J=6.0 Hz, 2H), 3.07 (s, 3H), 2.36-2.87 (s, 5H), 2.09-1.98 (m, 2H), 1.88-1.79 (m, 2H). LCMS (ESI) calcd. for C₂₃H₂₇F₂N₇O₂S [M+H]⁺ m/z 504.20, found 504.10.

### Example 72

### (R)-3-(4,4-difluoroazepan-1-yl)-5-methyl-6-(1-methyl-1H-pyrazol-4-yl)-N-(3-(S-methyl-N-(methylglycyl)sulfonimidoyl)phenyl)pyridazine-4-carboxamide

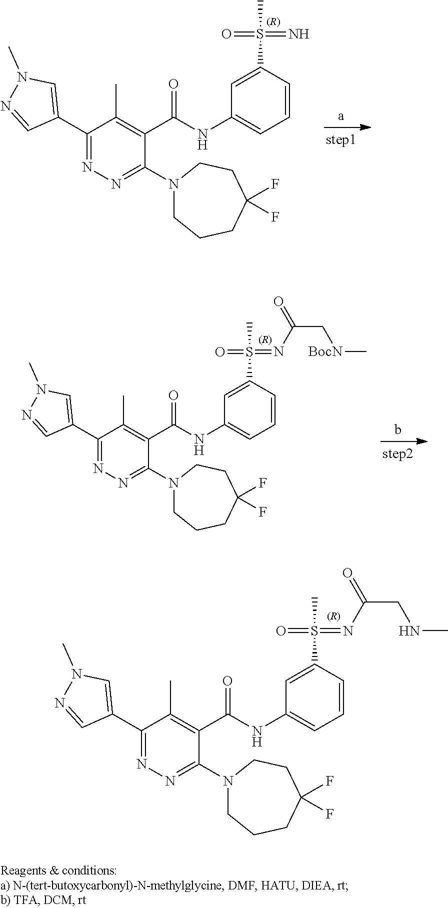

Step 1: tert-butyl (R)-(2-(((3-(3-(4,4-difluoroazepan-1-yl)-5-methyl-6-(1-methyl-1H-pyrazol-4-yl)pyridazine-4-carboxamido)phenyl)(methyl)(oxo)-λ⁶-sulfaneylidene)amino)-2-oxoethyl)(methyl)carbamate: A mixture of (R)-3-(4,4-difluoroazepan-1-yl)-5-methyl-6-(1-methyl-1H-pyrazol-4-yl)-N-(3-(S-methylsulfonimidoyl)phenyl)pyridazine-4-carboxamide (90 mg, 0.18 mmol), N-(tert-butoxycarbonyl)-N-methylglycine (68 mg, 0.36 mmol) and HATU (103 mg, 0.27 mmol) in DMF (3 mL) was added DIEA (70 mg, 0.54 mmol) at room temperature. The reaction mixture was stirred at room temperature for 3 hours. LCMS showed the reaction was completed. The mixture was diluted with water and extracted with EtOAc (3×30 mL). The combined organic layers were washed with brine, dried with Na₂SO₄, and concentrated under vacuum. The residue was purified by flash column chromatography on silica gel (eluting with MeOH/DCM, 0 to 10%) to give tert-butyl (R)-(2-(((3-(3-(4,4-difluoroazepan-1-yl)-5-methyl-6-(1-methyl-1H-pyrazol-4-yl)pyridazine-4-carboxamido)phenyl)(methyl)(oxo)-λ⁶-sulfaneylidene)amino)-2-oxoethyl)(methyl)carbamate (95 mg, 78.31%) as a yellow oil. LCMS (ESI) calcd. for C₃₁H₄₁F₂N₈O₅S [M+H]⁺ m/z 675.29, found 675.30.

Step 2: (R)-3-(4,4-difluoroazepan-1-yl)-5-methyl-6-(1-methyl-1H-pyrazol-4-yl)-N-(3-(S-methyl-N-(methylglycyl)sulfonimidoyl)phenyl)pyridazine-4-carboxamide: A solution of tert-butyl (R)-(2-(((3-(3-(4,4-difluoroazepan-1-yl)-5-methyl-6-(1-methyl-1H-pyrazol-4-yl)pyridazine-4-carboxamido)phenyl)(methyl)(oxo)-λ⁶-sulfaneylidene)amino)-2-oxoethyl)(methyl)carbamate (95 mg, 0.14 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 40% to 90% MeCN/H₂O containing 0.05% NH₃) to provide (R)-3-(4,4-difluoroazepan-1-yl)-5-methyl-6-(1-methyl-1H-pyrazol-4-yl)-N-(3-(S-methyl-N-(methylglycyl)sulfonimidoyl)phenyl)pyridazine-4-carboxamide (30.20 mg, 37.58%) as a white solid. ¹H NMR (400 MHz, CD₃OD) δ 8.48 (t, J=1.9 Hz, 1H), 8.06 (s, 1H), 8.02-7.95 (m, 1H), 7.91 (s, 1H), 7.89-7.81 (m, 1H), 7.71 (t, J=8.0 Hz, 1H), 4.00 (s, 3H), 3.86-3.79 (m, 2H), 3.73 (t, J=6.1 Hz, 2H), 3.47 (s, 3H), 3.40 (s, 2H), 2.44 (s, 3H), 2.41 (s, 3H), 2.39-2.29 (m, 2H), 2.15-2.07 (m, 2H), 1.99-1.95 (m, 2H). LCMS (ESI) calcd. for C₂₆H₃₃F₂N₈O₃S [M+H]⁺ m/z 575.24, found 575.20.

### Example 73

### (R)-N-(3-(N-(1-aminocyclobutane-1-carbonyl)-S-methylsulfonimidoyl)phenyl)-5-cyano-2-(4,4-difluoroazepan-1-yl)-4-methyl-6-(trifluoromethyl)nicotinamide

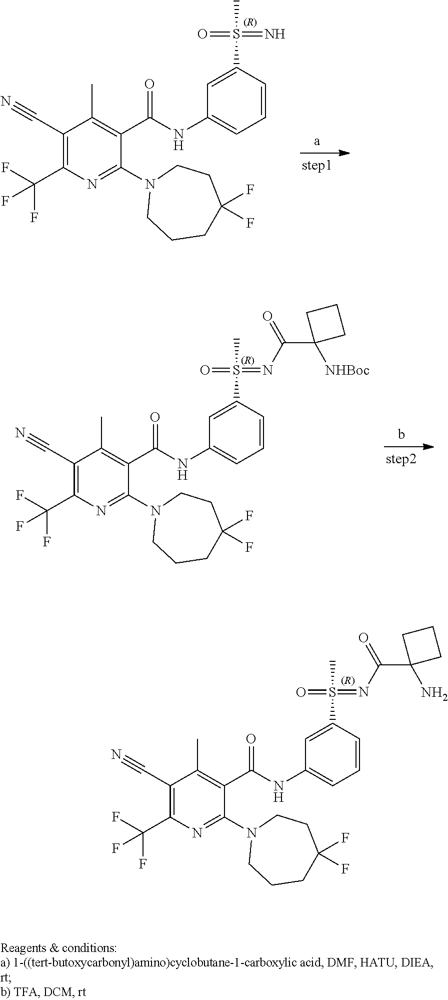

Step 1: tert-butyl (R)-(1-(((3-(5-cyano-2-(4,4-difluoroazepan-1-yl)-4-methyl-6-(trifluoromethyl)nicotinamido)phenyl)(methyl)(oxo)-λ⁶-sulfaneylidene)carbamoyl)cyclobutyl)carbamate: A solution of (R)-5-cyano-2-(4,4-difluoroazepan-1-yl)-4-methyl-N-(3-(S-methylsulfonimidoyl)phenyl)-6-(trifluoromethyl)nicotinamide (80 mg, 0.16 mmol), 1-((tert-butoxycarbonyl)amino)cyclobutane-1-carboxylic acid (50 mg, 0.23 mmol), HATU (118 mg, 0.31 mmol) and DIEA (60 mg, 0.47 mmol) in DMF (5 mL) was stirred at room temperature for 16 hours. LCMS showed the reaction was completed. The mixture was diluted with water (40 mL) and extracted with EtOAc (30 mL×3). The combined organic layers were washed with brine, dried over sodium sulfate, concentrated under vacuum. The residue was purified by flash column chromatography on silica gel (DCM/MeOH=95/5) to provide tert-butyl (R)-(1-(((3-(5-cyano-2-(4,4-difluoroazepan-1-yl)-4-methyl-6-(trifluoromethyl)nicotinamido)phenyl)(methyl)(oxo)-λ⁶-sulfaneylidene)carbamoyl)cyclobutyl)carbamate (60 mg, 54.19%) as brown oil. LCMS (ESI) calcd. for C₃₂H₃₈F₅N₆O₅S [M+H]⁺ m/z 713.26, found 713.15.

Step 2: (R)-N-(3-(N-(1-aminocyclobutane-1-carbonyl)-S-methylsulfonimidoyl)phenyl)-5-cyano-2-(4,4-difluoroazepan-1-yl)-4-methyl-6-(trifluoromethyl)nicotinamide: A solution of tert-butyl (R)-(1-(((3-(5-cyano-2-(4,4-difluoroazepan-1-yl)-4-methyl-6-(trifluoromethyl)nicotinamido)phenyl)(methyl)(oxo)-λ⁶-sulfaneylidene)carbamoyl)cyclobutyl)carbamate (60 mg, 0.08 mmol) in DCM (4 mL) was added TFA (0.4 mL) at room temperature. The reaction mixture was stirred at room temperature for 1 hour. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 30% to 95% MeCN/H₂O containing 0.1% formic acid) to provide (R)-N-(3-(N-(1-aminocyclobutane-1-carbonyl)-S-methylsulfonimidoyl)phenyl)-5-cyano-2-(4,4-difluoroazepan-1-yl)-4-methyl-6-(trifluoromethyl)nicotinamide (16.20 mg, 31.39%) as a white solid. ¹H NMR (400 MHz, DMSO-d₆) δ 11.14 (s, 1H), 8.38 (s, 1H), 7.91 (d, J=7.8 Hz, 1H), 7.77-7.65 (m, 2H), 3.75 (s, 2H), 3.65 (t, J=5.8 Hz, 2H), 3.50 (s, 3H), 2.47 (s, 3H), 2.45-2.37 (m, 2H), 2.36-2.23 (m, 2H), 2.10-1.96 (m, 2H), 1.94-1.72 (m, 6H). LCMS (ESI) calcd. for C₂₇H₃₀F₅N₆O₃S [M+H]⁺ m/z 613.20, found 613.00.

### Example 74

### (R)-2-(4,4-difluoroazepan-1-yl)-N-(3-(N-glycyl-S-methylsulfonimidoyl)phenyl)-4-methyl-5-(1-methyl-1H-pyrazol-4-yl)nicotinamide

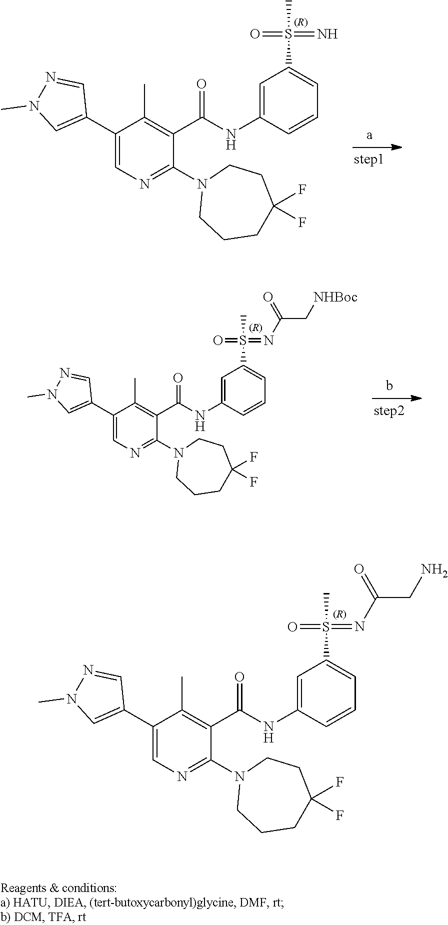

Step 1: tert-butyl (R)-(2-(((3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)amino)-2-oxoethyl)carbamate: A solution of (R)-2-(4,4-difluoroazepan-1-yl)-4-methyl-5-(1-methyl-1H-pyrazol-4-yl)-N-(3-(S-methylsulfonimidoyl)phenyl)nicotinamide (100 mg, 0.19 mmol), (tert-butoxycarbonyl)glycine (53 mg, 0.29 mmol), HATU (151 mg, 0.39 mmol) and DIEA (77 mg, 0.59 mmol) in DMF (5 mL) was stirred at room temperature for 16 hours. LCMS showed the reaction was completed. The mixture was diluted with water (40 mL) and extracted with EtOAc (30 mL×3). The combined organic layers were washed with brine, dried over sodium sulfate, concentrated under vacuum. The residue was purified by flash column chromatography on silica gel (DCM/MeOH=95/5) to provide tert-butyl (R)-(2-(((3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)amino)-2-oxoethyl)carbamate (70 mg, 47.99%) as yellow solid. LCMS (ESI) calcd. for C₃₁H₄₀F2N₇O₅S [M+H]⁺ m/z 660.28, found 660.25.

Step 2: (R)-2-(4,4-difluoroazepan-1-yl)-N-(3-(N-glycyl-S-methylsulfonimidoyl)phenyl)-4-methyl-5-(1-methyl-1H-pyrazol-4-yl)nicotinamide: A solution of tert-butyl (R)-(2-(((3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)amino)-2-oxoethyl)carbamate (70 mg, 0.11 mmol) in DCM (4 mL) was added TFA (0.4 mL) at room temperature. The reaction mixture was stirred at room temperature for 1 hour. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 30% to 80% MeCN/H₂O containing 0.05% NH₃) to provide (R)-2-(4,4-difluoroazepan-1-yl)-N-(3-(N-glycyl-S-methylsulfonimidoyl)phenyl)-4-methyl-5-(1-methyl-1H-pyrazol-4-yl)nicotinamide (20 mg, 21.33%) as a white solid. ¹H NMR (400 MHz, DMSO-d₆) δ 10.93 (s, 1H), 8.35 (s, 1H), 8.18 (s, 1H), 8.01 (d, J=6.8 Hz, 1H), 7.90 (s, 1H), 7.66 (d, J=6.7 Hz, 2H), 7.61 (s, 1H), 3.88 (s, 3H), 3.61 (s, 2H), 3.54 (t, J=6.0 Hz, 2H), 3.45 (s, 3H), 3.23 (s, 2H), 2.34-2.26 (m, 2H), 2.25 (s, 3H), 2.06-1.94 (m, 2H), 1.84-1.74 (m, 2H). LCMS (ESI) calcd. for C₂₆H₃₂F₂N₇O₃S [M+H]⁺ m/z 560.23, found 560.15.

### Example 75

### N-(3-((R)-N-(D-alanyl)-S-methylsulfonimidoyl)phenyl)-2-(4,4-difluoroazepan-1-yl)-4-methyl-5-(1-methyl-1H-pyrazol-4-yl)nicotinamide

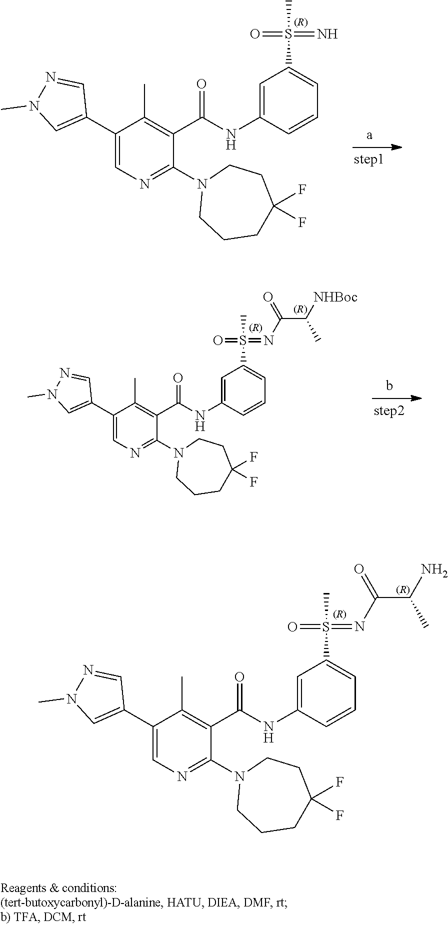

Step 1: tert-butyl ((R)-1-(((R)-(3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)amino)-1-oxopropan-2-yl)carbamate: A mixture of (R)-2-(4,4-difluoroazepan-1-yl)-4-methyl-5-(1-methyl-1H-pyrazol-4-yl)-N-(3-(S-methylsulfonimidoyl)phenyl)nicotinamide (100 mg, 0.19 mmol), (tert-butoxycarbonyl)-D-alanine (53 mg, 0.29 mmol) and HATU (144 mg, 0.38 mmol) in DMF (3 mL) was added DIEA (74 mg, 0.57 mmol) at room temperature. The reaction mixture was stirred at room temperature for 3 hours. LCMS showed the reaction was completed. The mixture was diluted with water and extracted with EtOAc (3×30 mL). The combined organic layers were washed with brine, dried with Na₂SO₄, and concentrated under vacuum. The residue was purified by flash column chromatography on silica gel (eluting with MeOH/DCM, 0 to 10%) to give tert-butyl ((R)-1-(((R)-(3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)amino)-1-oxopropan-2-yl)carbamate (90 mg, 72.31%) as a yellow solid. LCMS (ESI) calcd. for C₃₂H₄₂F₂N₇O₅S [M+H]⁺ m/z 674.29, found 675.20.

Step 2: N-(3-((R)-N-(D-alanyl)-S-methylsulfonimidoyl)phenyl)-2-(4,4-difluoroazepan-1-yl)-4-methyl-5-(1-methyl-1H-pyrazol-4-yl)nicotinamide: A solution of tert-butyl ((R)-1-(((R)-(3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)amino)-1-oxopropan-2-yl)carbamate (95 mg, 0.14 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 40% to 90% MeCN/H₂O containing 0.05% NH₃) to provide N-(3-((R)-N-(D-alanyl)-S-methylsulfonimidoyl)phenyl)-2-(4,4-difluoroazepan-1-yl)-4-methyl-5-(1-methyl-1H-pyrazol-4-yl)nicotinamide (20.20 mg, 27.58%) as a white solid. ¹H NMR (400 MHz, DMSO-d₆) δ 10.94 (s, 1H), 8.38 (s, 1H), 8.18 (s, 1H), 7.98 (dd, J=7.1, 2.1 Hz, 1H), 7.90 (s, 1H), 7.68 (dd, J=10.8, 4.6 Hz, 2H), 7.61 (s, 1H), 3.88 (s, 3H), 3.62-3.58 (m, 2H), 3.54 (t, J=6.1 Hz, 2H), 3.45 (s, 3H), 3.29 (d, J=6.9 Hz, 1H), 2.35-2.26 (m, 2H), 2.25 (s, 3H), 2.04-1.94 (s, 2H), 1.82-1.76 (m, 2H), 1.18 (d, J=6.9 Hz, 3H). LCMS (ESI) calcd. for C₂₇H₃₄F₂N₇O₃S [M+H]⁺ m/z 574.24, found 574.00.

### Example 76

### N-(3-((R)-N-(L-alanyl)-S-methylsulfonimidoyl)phenyl)-2-(4,4-difluoroazepan-1-yl)-4-methyl-5-(1-methyl-1H-pyrazol-4-yl)nicotinamide formate

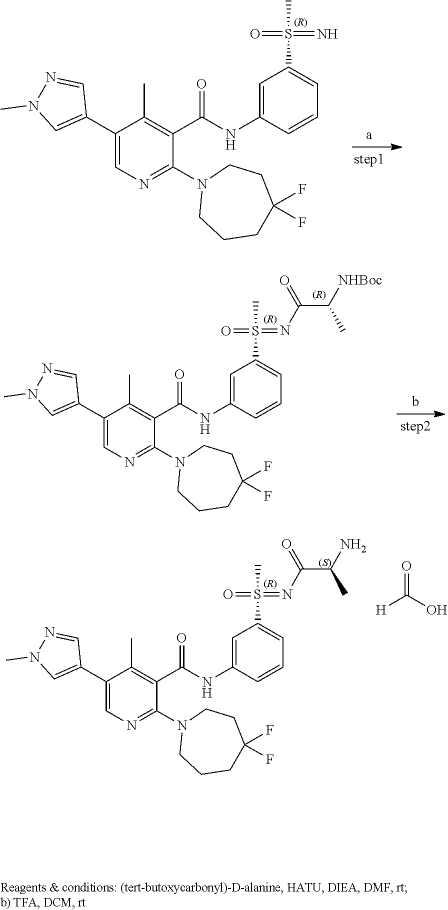

Step 1: tert-butyl ((S)-1-(((R)-(3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)amino)-1-oxopropan-2-yl)carbamate: To a solution of (R)-2-(4,4-difluoroazepan-1-yl)-4-methyl-5-(1-methyl-1H-pyrazol-4-yl)-N-(3-(S-methylsulfonimidoyl)phenyl)nicotinamide (100 mg, 0.20 mmol), (tert-butoxycarbonyl)-L-alanine (95 mg, 0.50 mmol) and HATU (114 mg, 0.30 mmol) in DMF (3 mL) and was added DIEA (77 mg, 0.60 mmol). Then the mixture was stirred at 25° C. for 3 hours. LCMS showed the reaction was completed. The mixture was diluted with water and extracted with EtOAc (3×6 mL). The combined organic layers were washed with brine, dried with Na₂SO₄, and concentrated under vacuum. The residue was purified by silica gel column chromatography (eluting with MeOH/DCM, 0 to 10%) to give tert-butyl ((S)-1-(((R)-(3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)amino)-1-oxopropan-2-yl)carbamate (110 mg, 781.72%) as a yellow oil. LCMS (ESI) calcd. for C₃₂H₄₂F₂N₇O₅S [M+H]⁺ m/z 674.29, found 674.20.

Step 2: N-(3-((R)-N-(L-alanyl)-S-methylsulfonimidoyl)phenyl)-2-(4,4-difluoroazepan-1-yl)-4-methyl-5-(1-methyl-1H-pyrazol-4-yl)nicotinamide formate: A solution of tert-butyl ((S)-1-(((R)-(3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)amino)-1-oxopropan-2-yl)carbamate (110 mg, 0.16 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (10 mL×3). The combined organic phases were washed with brine, dried over sodium sulfate, concentrated under vacuum. The residue was purified by prep-HPLC (Gemini 5 um C₁₈ column, 150*19 mm, eluting with 20% to 45% ACN/H₂O containing 0.1% FA) to provide N-(3-((R)-N-(L-alanyl)-S-methylsulfonimidoyl)phenyl)-2-(4,4-difluoroazepan-1-yl)-4-methyl-5-(1-methyl-1H-pyrazol-4-yl)nicotinamide formate (35.80 mg, 36.14% o) as a white solid. ¹H NMR (400 MHz, CD₃OD, ppm) δ 8.64 (d, J=2.0 Hz, 1H), 8.55 (s, 1H), 8.19 (s, 1H), 7.95-7.86 (m, 1H), 7.84-7.74 (m, 2H), 7.71 (t, J=8.0 Hz, 1H), 7.60 (s, 1H), 3.97 (s, 3H), 3.96-3.90 (m, 1H), 3.69-3.64 (m, 2H), 3.62 (t, J=6.2 Hz, 2H), 3.53 (d, J=10.4 Hz, 3H), 2.36 (s, 3H), 2.34-2.23 (m, 2H), 2.12-2.00 (m, 2H), 1.95-1.85 (m, 2H), 1.59 (t, J=6.9 Hz, 3H). LCMS (ESI) calcd. for C₂₇H₃₃F₂N₇O₃SNa [M+Na]⁺ m/z 596.22, found 595.95.

### Example 77

### (R)-5-chloro-2-(4,4-difluoroazepan-1-yl)-N-(3-(S-methylsulfonimidoyl)phenyl)-4-(trifluoromethyl)benzamide

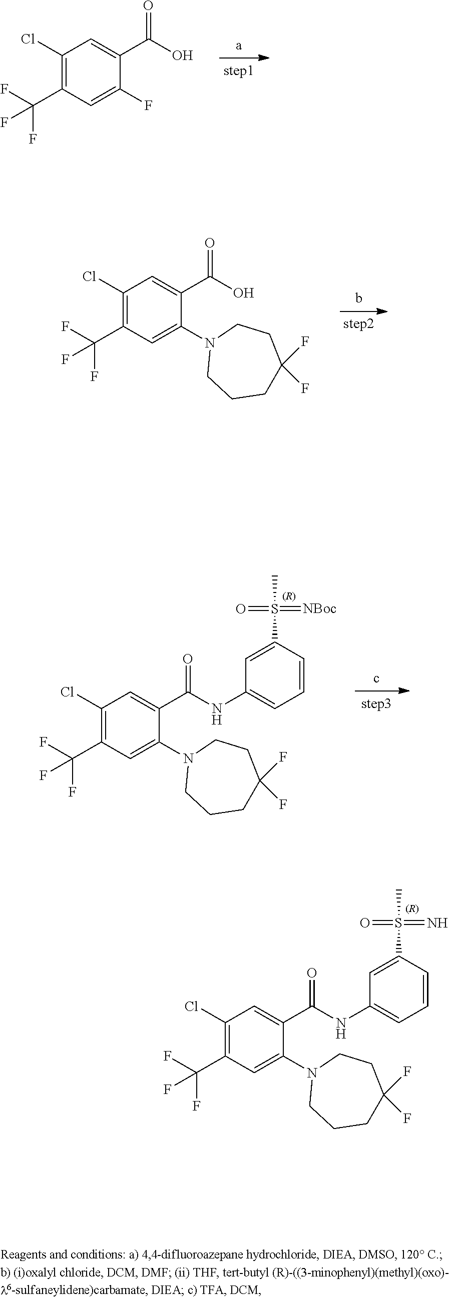

Step 1: 5-chloro-2-(4,4-difluoroazepan-1-yl)-4-(trifluoromethyl)benzoic acid: A mixture of 5-chloro-2-fluoro-4-(trifluoromethyl)benzoic acid (1.2 g, 4.96 mmol), 4,4-difluoroazepane hydrochloride (933 mg, 5.45 mmol) and DIEA (1.9 g, 14.88 mmol) in DMSO (20 mL) was heated at 120° C. for 16 hours. After the reaction was completed, the mixture was cooled to room temperature. The resulting solution was diluted with water (60 mL) and extracted with EtOAc (60 mL×3). The combined organic layers were washed with brine, dried over Na₂SO₄, concentrated under vacuum. The residue was purified by flash column chromatography on silica gel (PE/EtOAc=1/4) to provide 5-chloro-2-(4,4-difluoroazepan-1-yl)-4-(trifluoromethyl)benzoic acid (800 mg, 45.20%) as a yellow solid. LCMS (ESI) calcd. for C₁₄H₁₄ClF₅NO₂ [M+H]⁺ m/z 358.07, found 357.90.

Step 2: tert-butyl (R)-((3-(5-chloro-2-(4,4-difluoroazepan-1-yl)-4-(trifluoromethyl)benzamido)phenyl)(methyl)(oxo)-λ⁶-sulfaneylidene)carbamate: To a solution of 5-chloro-2-(4,4-difluoroazepan-1-yl)-4-(trifluoromethyl)benzoic acid (150 mg, 0.42 mmol) in DCM (5 mL) was added Oxalyl chloride (96 mg, 0.76 mmol) and DMF (0.02 mL). The mixture was stirred at room temperature for 1 h. Then the mixture was concentrated in vacuum. The residue was diluted with THF (5 mL) and added to a solution of tert-butyl (R)-((3-aminophenyl)(methyl)(oxo)-λ⁶-sulfaneylidene)carbamate (125 mg, 0.46 mmol) and DIEA (163 mg, 1.26 mmol) in THE (3 mL). The mixture was stirred at room temperature for 1 hour. After the reaction was completed, the mixture was concentrated under vacuum. The residue was purified by flash column chromatography on silica gel (PE/EtOAc=1/1) to provide tert-butyl (R)-((3-(5-chloro-2-(4,4-difluoroazepan-1-yl)-4-(trifluoromethyl)benzamido)phenyl)(methyl)(oxo)-λ⁶-sulfaneylidene)carbamate (100 mg, 49.02%) as a yellow oil. LCMS (ESI) calcd. for C₂₁H₂₂ClF₅N₃O₂S [M+H-Boc]⁺ m/z 510.11, found 510.00.

Step 3: (R)-5-chloro-2-(4,4-difluoroazepan-1-yl)-N-(3-(S-methylsulfonimidoyl)phenyl)-4-(trifluoromethyl)benzamide: To a solution of tert-butyl (R)-((3-(5-chloro-2-(4,4-difluoroazepan-1-yl)-4-(trifluoromethyl)benzamido)phenyl)(methyl)(oxo) λ⁶-sulfaneylidene)carbamate (100 mg, 0.16 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 30% to 90% MeCN/H₂O containing 0.05% NH₃) to provide (R)-5-chloro-2-(4,4-difluoroazepan-1-yl)-N-(3-(S-methylsulfonimidoyl)phenyl)-4-(trifluoromethyl)benzamide (56 mg, 67.47%) as a white solid. ¹H NMR (400 MHz, DMSO-d₆) δ 10.87 (s, 1H), 8.34 (t, J=1.7 Hz, 1H), 7.92 (d, J=8.0 Hz, 1H), 7.73-7.65 (m, 2H), 7.60 (t, J=7.9 Hz, 1H), 7.34 (s, 1H), 4.23 (s, 1H), 3.44-3.35 (m, 4H), 3.05 (s, 3H), 2.29-2.17 (m, 2H), 2.10-1.98 (m, 2H), 1.85-1.72 (m, 2H). LCMS (ESI) calcd. for C₂₁H₂₂ClF₅N₃O₂S [M+H]⁺ m/z 510.11, found 510.05.

### Example 78

### (R)-6-(4,4-difluoroazepan-1-yl)-2-methyl-3-(1-methyl-1H-pyrazol-4-yl)-N-(3-(S-methylsulfonimidoyl)phenyl)benzamide

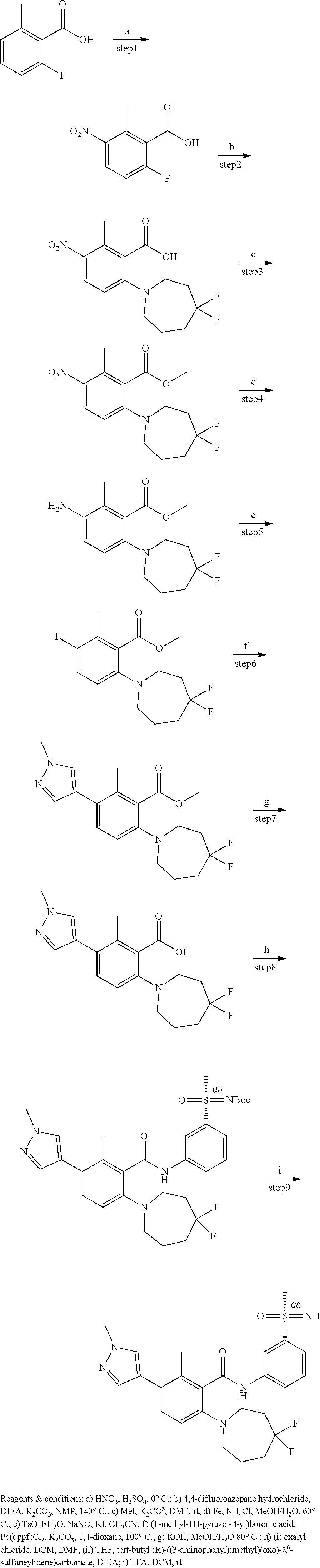

Step 1: 6-fluoro-2-methyl-3-nitrobenzoic acid: To a solution of fuming HNO₃ (0.69 mL, 15.6 mmol) in sulfuric acid (4.4 mL) was added a solution of 2-fluoro-6-methylbenzoic acid (1.6 g, 10.4 mmol) in concentrated sulfuric acid (15 mL) at 0° C. and the mixture was stirred at 0° C. for 30 min. Then the mixture was poured into ice water (50 mL). The precipitate was collected by filtration, washed with water, and dried under vacuum to provide 6-fluoro-2-methyl-3-nitrobenzoic acid (1.7 g, 82.1%) as a yellow solid. LCMS (ESI) calcd. for C₈H₅FNO₄ [M−H]-m/z 198.02, found 198.05.

Step 2: 6-(4,4-difluoroazepan-1-yl)-2-methyl-3-nitrobenzoic acid: A mixture of 6-fluoro-2-methyl-3-nitrobenzoic acid (1.7 g, 8.54 mmol) and 4,4-difluoroazepane hydrochloride (1.76 g, 10.24 mmol) in NMP (30 mL) was added DIEA (5.52 g, 42.68 mmol) and K₂CO₃ (5.90 g, 42.68 mmol) at room temperature. The reaction mixture was heated at 140° C. for 16 hours. After the reaction was completed, the solution was adjusted to pH=5-6 with 1N HCl and extracted with DCM (150 mL×2). The combined organic layers were washed with brine (150 mL×3), dried over sodium sulfate, then concentrated and purified by flash column chromatography on silica gel (DCM/MeOH=20/1 to 10/1) to give 6-(4,4-difluoroazepan-1-yl)-2-methyl-3-nitrobenzoic acid (1.6 g, 55.9%) as a white solid. LCMS (ESI) calcd. for C₁₄H₁₇F₂N₂O₄ [M+H]⁺ m/z 315.12, found 315.05.

Step 3: methyl 6-(4,4-difluoroazepan-1-yl)-2-methyl-3-nitrobenzoate: To a solution of 6-(4,4-difluoroazepan-1-yl)-2-methyl-3-nitrobenzoic acid (1.6 g, 5.09 mmol) in DMF (20 mL) was added K₂CO₃ (1.41 g, 10.18 mmol) and Mel (1.08 g, 7.64 mmol). The mixture was stirred at room temperature for 12 hours. After the reaction was completed, solution was diluting with DCM (100 mL) and washed with brine (100 mL×3), dried over sodium sulfate, then concentrated under vacuum. The residue was purified by flash column chromatography on silica gel (PE/EtOAc=1/1 to 1/3) to provide methyl 6-(4,4-difluoroazepan-1-yl)-2-methyl-3-nitrobenzoate (1.5 g, 89.7%) as a light-yellow solid. LCMS (ESI) calcd. for C₁₅H₁₉F₂N₂O₄ [M+H]⁺ m/z 329.13, found 329.10.

Step 4: methyl 3-amino-6-(4,4-difluoroazepan-1-yl)-2-methylbenzoate: A solution of methyl 6-(4,4-difluoroazepan-1-yl)-2-methyl-3-nitrobenzoate (1.4 g, 4.26 mmol) in MeOH (30 mL) and water (10 mL) was added NH₄Cl (1.60 g, 29.85 mmol), Fe (1.19 g, 21.32 mmol). The mixture was heated to 60° C. for 1 hour. After the reaction was completed, the mixture was filtered through celite. The filtrate was diluted with water (100 mL) and extracted with EtOAc (50 mL×3). The combined organic layers were washed with brine, dried over sodium sulfate, concentrated under vacuum to provide methyl 3-amino-6-(4,4-difluoroazepan-1-yl)-2-methylbenzoate (1.2 g, 94.3%) as a yellow solid. LCMS (ESI) calcd. for C₁₅H₂₁F₂N₂O₂ [M+H]+m/z 299.16, found 299.10.

Step 5: methyl 6-(4,4-difluoroazepan-1-yl)-3-iodo-2-methylbenzoate: To a solution of TsOH·H₂O (1.53 g, 8.04 mmol) and methyl 3-amino-6-(4,4-difluoroazepan-1-yl)-2-methylbenzoate (800 mg, 2.68 mmol) in MeCN (30 mL) was added a solution of NaNO₂ (370 mg, 5.36 mmol) and KI (1.11 g, 6.70 mmol) in H₂O (5 mL) at 0° C. The mixture was stirred at room temperature for 2.5 hours. After the reaction was completed, the mixture was quenched with water (100 mL), adjusted to pH=8-9 with saturated aqueous NaHCO₃. Then the solution was extracted with EtOAc (200 mL×3). The combined organic layers were washed with brine, dried over sodium sulfate, concentrated under vacuum. The residue was purified by flash column chromatography on silica gel (PE/EtOAc=10/1 to 1/1) to provide methyl 6-(4,4-difluoroazepan-1-yl)-3-iodo-2-methylbenzoate (500 mg, 45.6%) as yellow solid. LCMS (ESI) calcd. for C₁₅H₁₉F₂INO₂ [M+H]⁺ m/z 410.05, found 410.00.

Step 6: methyl 6-(4,4-difluoroazepan-1-yl)-2-methyl-3-(1-methyl-1H-pyrazol-4-yl)benzoate: A mixture of methyl 6-(4,4-difluoroazepan-1-yl)-3-iodo-2-methylbenzoate (400 mg, 0.98 mmol) and (1-methyl-1H-pyrazol-4-yl)boronic acid (184 mg, 1.47 mmol) in 1,4-dioxane/H₂O (4/1, 10 mL) was added potassium carbonate (405 mg, 2.93 mmol) and Pd(dppf)Cl₂ (143 mg, 0.20 mmol). Then the mixture was heated at 100° C. for 6 hours under an atmosphere of N₂. The reaction was monitored by LCMS. After the reaction was completed, the mixture was cooled to room temperature. The resulting solution was diluted with water (20 mL) and extracted with DCM (20 mL×3). The combined organic layers were washed with brine, dried over Na₂SO₄, concentrated under vacuum. The residue was purified by flash column chromatography on silica gel (PE/EtAOc=1/1) to give methyl 6-(4,4-difluoroazepan-1-yl)-2-methyl-3-(1-methyl-1H-pyrazol-4-yl)benzoate (300 mg, 84.5%) as a yellow oil. LCMS (ESI) calcd. for C₁₉H₂₄F₂N₃O₂ [M+H]⁺ m/z 364.19, found 364.10.

Step 7: 6-(4,4-difluoroazepan-1-yl)-2-methyl-3-(1-methyl-1H-pyrazol-4-yl)benzoic acid: To a solution of methyl 6-(4,4-difluoroazepan-1-yl)-2-methyl-3-(1-methyl-1H-pyrazol-4-yl)benzoate (200 mg, 0.55 mmol) in MeOH/H₂O (1/1, 10 mL) was added KOH (309 mg, 5.50 mmol) at room temperature. The reaction mixture was heated at 80° C. for 18 hours. After the reaction was completed, the mixture was concentrated. The residue was adjusted to pH=3-4 with aqueous HCl (1M). Then the solution was extracted with EtOAc (30 mL×3). The combined organic layers were washed with brine, dried over sodium sulfate, concentrated under vacuum to provide 6-(4,4-difluoroazepan-1-yl)-2-methyl-3-(1-methyl-1H-pyrazol-4-yl)benzoic acid (150 mg, 78.00%) as a white solid. LCMS (ESI) calcd. for C₁₈H₂₂F₂N₃O₂ [M+H]⁺ m/z 350.17, found 350.10.

Step 8: tert-butyl (R)-((3-(6-(4,4-difluoroazepan-1-yl)-2-methyl-3-(1-methyl-1H-pyrazol-4-yl)benzamido)phenyl)(methyl)(oxo)-λ⁶-sulfaneylidene)carbamate: To a solution of 6-(4,4-difluoroazepan-1-yl)-2-methyl-3-(1-methyl-1H-pyrazol-4-yl)benzoic acid (150 mg, 0.43 mmol) in DCM (5 mL) was added oxalyl chloride (163 mg, 1.29 mmol) and DMF (20 L). The mixture was stirred at room temperature for 0.5 hour. Then the mixture was concentrated under vacuum. The residue was dissolved in THE (5 mL) and added to a solution of tert-butyl (R)-((3-aminophenyl)(methyl)(oxo)-16-sulfaneylidene)carbamate (139 mg, 0.52 mmol) and TEA (217 mg, 2.15 mmol) in THE (5 mL). The mixture was stirred at room temperature for 1 hour. After the reaction was completed, the mixture was concentrated under vacuum. The residue was purified by flash column chromatography on silica gel (PE/EtOAc=2/1) to give tert-butyl (R)-((3-(6-(4,4-difluoroazepan-1-yl)-2-methyl-3-(1-methyl-1H-pyrazol-4-yl)benzamido)phenyl)(methyl)(oxo)-λ⁶-sulfaneylidene)carbamate (150 mg, 58.1%) as a yellow oil. LCMS (ESI) calcd. for C₃₀H₃₈F2N₅O₄S [M+H]⁺ m/z 602.26, found 602.15.

Step 9: (R)-6-(4,4-difluoroazepan-1-yl)-2-methyl-3-(1-methyl-1H-pyrazol-4-yl)-N-(3-(S-methylsulfonimidoyl)phenyl)benzamide: A solution of tert-butyl (R)-((3-(6-(4,4-difluoroazepan-1-yl)-2-methyl-3-(1-methyl-1H-pyrazol-4-yl)benzamido)phenyl)(methyl)(oxo)-λ⁶-sulfaneylidene)carbamate (150 mg, 0.25 mmol) in DCM (5 mL) was added TFA (0.5 mL) at room temperature. The reaction mixture was stirred at room temperature for 2 hours. After the reaction was completed, the solution was diluted with water (10 mL) and extracted with DCM (10 mL×3). The combined organic layers were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.1% NH₄OH) to obtain (R)-6-(4,4-difluoroazepan-1-yl)-2-methyl-3-(1-methyl-1H-pyrazol-4-yl)-N-(3-(S-methylsulfonimidoyl)phenyl)benzamide (42.5 mg, 33.9%) as a white solid. ¹H NMR (400 MHz, DMSO-d₆) δ 10.67 (s, 1H), 8.44 (s, 1H), 7.88 (d, J=7.3 Hz, 2H), 7.66-7.51 (m, 3H), 7.35 (d, J=8.3 Hz, 1H), 7.14 (d, J=8.3 Hz, 1H), 4.19 (s, 1H), 3.88 (s, 3H), 3.17-3.08 (m, 4H), 3.03 (s, 3H), 2.28 (s, 3H), 2.13-1.88 (m, 4H), 1.68 (m, 2H). LCMS (ESI) calcd. for C₂₅H₃₀F₂N₅O₂S [M+H]⁺ m/z 502.21, found 502.15.

### Example 79

### 3-(Azepan-1-yl)-5-methyl-N-(3-(S-methylsulfonimidoyl)phenyl)-6-(3-methylthiophen-2-yl)pyridazine-4-carboxamide

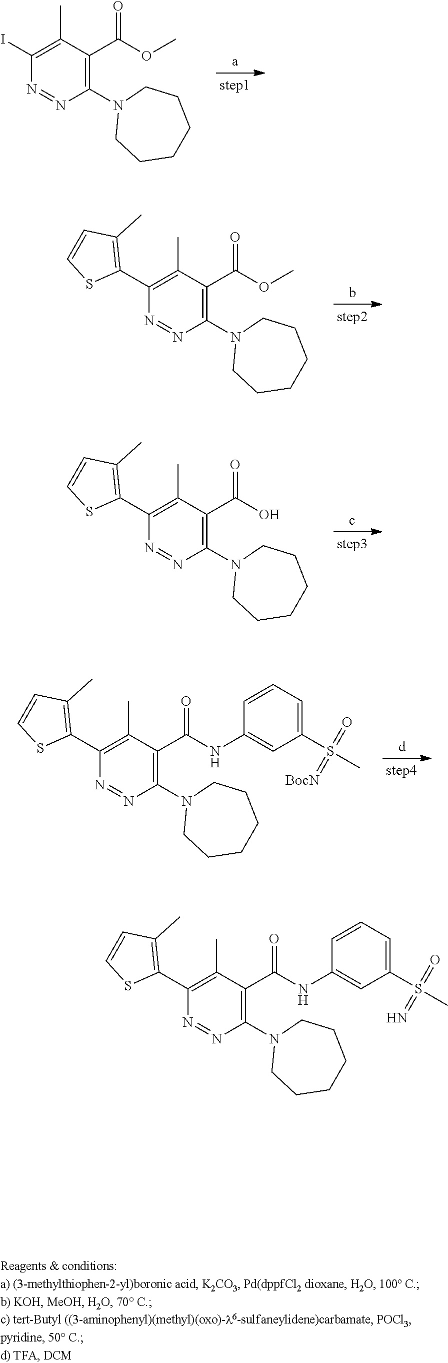

Step 1: methyl 3-(azepan-1-yl)-5-methyl-6-(3-methylthiophen-2-yl)pyridazine-4-carboxylate: A mixture of methyl 3-(azepan-1-yl)-6-iodo-5-methylpyridazine-4-carboxylate (450 mg, 1.20 mmol) and (3-methylthiophen-2-yl)boronic acid (340 mg, 2.400 mmol) in 1,4-dioxane/H₂O (4/1, 20 mL), potassium carbonate (324 mg, 2.400 mmol) and [1,1′-Bis(diphenylphosphino)ferrocene]dichloropalladium (II) (87 mg, 0.12 mmol) was heated at 100° C. for 6 hours under an atmosphere of N₂. After the reaction was completed, the mixture was diluted with water (20 mL) and extracted with DCM (20 mL×3). The combine organic phases were washed with brine, dried over Na₂SO₄, concentrated under vacuum. The residue was purified by flash column chromatography on silica gel (PE/EtOAc=1/1) to give methyl 3-(azepan-1-yl)-5-methyl-6-(3-methylthiophen-2-yl)pyridazine-4-carboxylate (320 mg, 77.29%) as a white solid. LCMS (ESI) calcd. for C₁₈H₂₄N₃O₂S [M+H]⁺ m/z 346.15, found 346.00.

Step 2: 3-(azepan-1-yl)-5-methyl-6-(3-methylthiophen-2-yl)pyridazine-4-carboxylic acid: To a solution of methyl 3-(azepan-1-yl)-5-methyl-6-(3-methylthiophen-2-yl)pyridazine-4-carboxylate (270 mg, 0.783 mmol) in MeOH/H₂O (1/1, 8 mL) was added KOH (438 mg, 7.83 mmol) at room temperature. The mixture was heated at 70° C. for 5 hours. After the reaction was completed, the mixture was cooled to room temperature and concentrated to remove most MeOH. The aqueous phase was adjusted to pH=3-4 with 1N HCl and extracted with EtOAc (20 mL×3). The combined organic phases were washed with brine, dried with Na₂SO₄, and concentrated under reduced pressure to give 3-(azepan-1-yl)-5-methyl-6-(3-methylthiophen-2-yl)pyridazine-4-carboxylic acid (180 mg, 69.49%) as a white solid. LCMS (ESI) calcd. for C₁₇H₂₂N₃O₂S [M+H]⁺ m/z 332.14, found 332.05.

Step 3: tert-butyl ((3-(3-(azepan-1-yl)-5-methyl-6-(3-methylthiophen-2-yl)pyridazine-4-carboxamido)phenyl)(methyl)(oxo)-λ⁶-sulfaneylidene)carbamate: To a solution of 3-(azepan-1-yl)-5-methyl-6-(3-methylthiophen-2-yl)pyridazine-4-carboxylic acid (130 mg, 0.392 mmol) and tert-butyl ((3-aminophenyl)(methyl)(oxo)-λ⁶-sulfaneylidene)carbamate (159 mg, 0.589 mmol) in pyridine (4 mL) was added POCl₃ (305 L) at 50° C. The mixture was heated at 50° C. for 1 hour. LCMS showed the reaction was completed. The final mixture was quenched with water and extracted with EtOAc. The combined organic phases were washed with water and brine, dried with sodium sulfate, concentrated, and purified by silica gel column chromatography (eluting with EtOAc/PE=1/2) to give tert-butyl ((3-(3-(azepan-1-yl)-5-methyl-6-(3-methylthiophen-2-yl)pyridazine-4-carboxamido)phenyl)(methyl)(oxo)-λ⁶-sulfaneylidene)carbamate (50 mg, 21.83%) as a yellow oil. LCMS (ESI) calcd. for C₂₉H₃₈N₅O₄S₂ [M+H]⁺ m/z 584.23, found 584.15.

Step 4: 3-(azepan-1-yl)-5-methyl-N-(3-(S-methylsulfonimidoyl)phenyl)-6-(3-methylthiophen-2-yl)pyridazine-4-carboxamide: To a solution of the tert-rt-butyl ((3-(3-(azepan-1-yl)-5-methyl-6-(3-methylthiophen-2-yl)pyridazine-4-carboxamido)phenyl)(methyl)(oxo)-λ⁶-sulfaneylidene)carbamate (50 mg, 0.086 mmol) in DCM (2 mL) was added TFA (0.4 mL) at 0° C. The mixture was stirred room temperate for 2 hours. Then the mixture was concentrated and the residue was purified by prep-HPLC (Column: Gemini-C₁₈, 150×21.2 mm, 5 um; Mobile Phase: ACN-H₂O (0.1% FA), Gradient: 30%-40%-70%) to give 3-(azepan-1-yl)-5-methyl-N-(3-(S-methylsulfonimidoyl)phenyl)-6-(3-methylthiophen-2-yl)pyridazine-4-carboxamide (3 mg, 12.20%) as a white solid. ¹H NMR (400 MHz, DMSO-d₆) δ 11.09 (s, 1H), 8.30 (s, 1H), 7.89-7.88 (m, 1H), 7.70-7.68 (m, 1H), 7.60-7.58 (m, 2H), 7.05-7.03 (m, 1H), 4.24 (s, 1H), 3.70-3.68 (m, 4H), 3.07-3.05 (m, 4H), 2.09-2.07 (m, 4H), 1.77 (s, 3H), 1.51 (s, 3H), 1.23 (s, 3H). LCMS (ESI) calcd. for C₂₄H₃₀N₅O₂S₂ [M+H]⁺ m/z 484.18, found 484.15.

### Example 80

### 6-(cyclopropylmethyl)-N-(3-(S-methylsulfonimidoyl)phenyl)-2-(6-azaspiro[2.5]octan-6-yl)nicotinamide

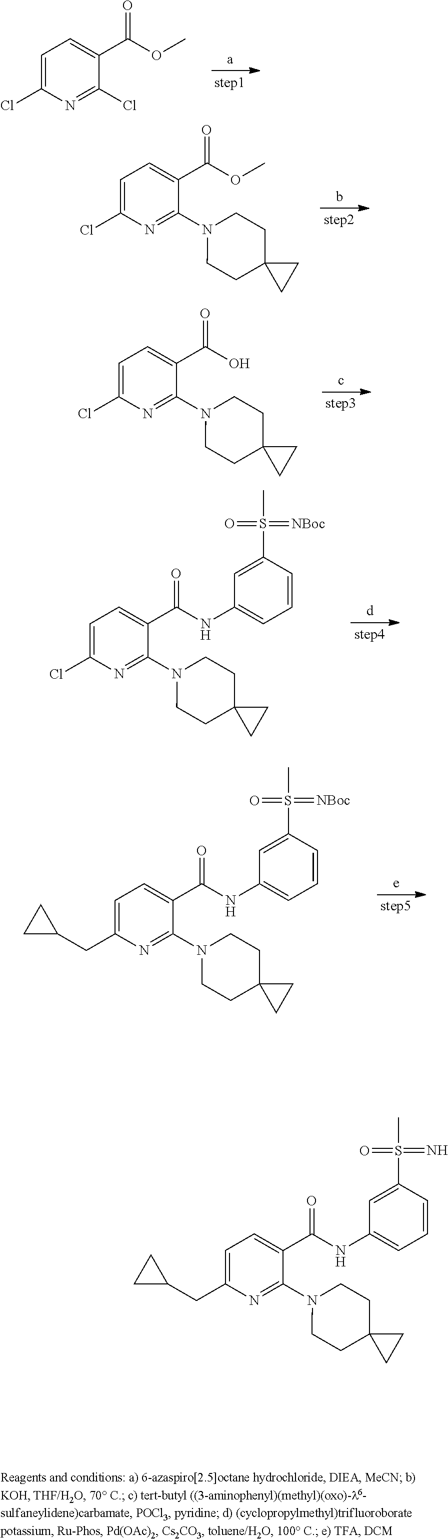

Step 1: methyl 6-chloro-2-(6-azaspiro[2.5]octan-6-yl)nicotinate: A mixture of methyl 2,6-dichloronicotinate (2.0 g, 9.75 mmol) and 6-azaspiro[2.5]octane hydrochloride (1.43 g, 9.75 mmol) in MeCN (20 mL) was added DIEA (2.52 g, 19.5 mmol) at room temperature. The reaction mixture was stirred at 25° C. for 16 hours. After the reaction was completed, the mixture was diluted with water (40 mL) and extracted with DCM (40 mL×3). The combined organic phases were washed with brine, dried over sodium sulfate, concentrated under vacuum. The residue was purified by flash column chromatography on silica gel (PE/EtOAc=3/1) to provide methyl 6-chloro-2-(6-azaspiro[2.5]octan-6-yl)nicotinate (1.67 g, 61.1%) as a yellow solid. LCMS (ESI) calcd. for C₁₄H₁₈ClN₂O₂ [M+H]⁺ m/z 281.11, found 281.05.

Step 2: 6-chloro-2-(6-azaspiro[2.5]octan-6-yl)nicotinic acid: To a solution methyl 6-chloro-2-(6-azaspiro[2.5]octan-6-yl)nicotinate (1.6 g, 5.69 mmol) in THF/H₂O (1/1, 15 mL) was added KOH (3.18 g, 56.9 mmol) at room temperature. The mixture was heated at 70° C. for 6 hours. After the reaction was completed, the mixture was cooled to room temperature and extracted with DCM (20 mL). Then the aqueous phase was adjusted to pH=3 with 1N HCl and extracted with DCM (20 mL×3). The combined organic phases were dried with Na₂SO₄ and concentrated under reduced pressure to give 6-chloro-2-(6-azaspiro[2.5]octan-6-yl)nicotinic acid (1.4 g, 79.56%) as a yellow solid. LCMS (ESI) calcd. for C₁₃H₁₆ClN₂O₂ [M+H]⁺ m/z 267.09, found 266.95.

Step 3: tert-butyl ((3-(6-chloro-2-(6-azaspiro[2.5]octan-6-yl)nicotinamido)phenyl)(methyl)(oxo)-λ⁶-sulfaneylidene)carbamate: A mixture of 6-chloro-2-(6-azaspiro[2.5]octan-6-yl)nicotinic acid (300 mg, 1.12 mmol) and tert-butyl ((3-aminophenyl)(methyl)(oxo)-λ⁶-sulfaneylidene)carbamate (364 mg, 1.34 mmol) in pyridine (5 mL) was added POCl₃ (100 L) dropwise at 0° C. The reaction solution was stirred at 0° C. for 1 hour. After the reaction was completed, the resulting solution was diluted with water (20 mL) and extracted with DCM (20 mL×3). The combined organic phases were washed with brine, dried over sodium sulfate, concentrated under vacuum. The residue was purified by flash column chromatography on silica gel (PE/EtOAc=2/1) to give tert-butyl ((3-(6-chloro-2-(6-azaspiro[2.5]octan-6-yl)nicotinamido)phenyl)(methyl)(oxo)-λ⁶-sulfaneylidene)carbamate (200 mg, 49.8%) as a yellow oil. LCMS (ESI) calcd. for C₂₅H₃₂ClN₄O₄S [M+Na]⁺ m/z 519.19, found 519.10.

Step 4: tert-butyl ((3-(6-(cyclopropylmethyl)-2-(6-azaspiro[2.5]octan-6-yl)nicotinamido)phenyl)(methyl)(oxo)-λ⁶-sulfaneylidene)carbamate: A mixture tert-butyl ((3-(6-chloro-2-(6-azaspiro[2.5]octan-6-yl)nicotinamido)phenyl)(methyl)(oxo)-λ⁶-sulfaneylidene)carbamate (180 mg, 0.34 mmol), (cyclopropylmethyl)trifluoroborate potassium (110 mg, 0.68 mmol), cesium carbonate (332 mg, 1.02 mmol), Pd(OAc)₂ (23 mg, 0.10 mmol) and Ru-phos (32 mg, 0.068 mmol) in toluene/H₂O (10/1, 11 mL) was heated at 100° C. for 16 h under an atmosphere of N₂. After the reaction was completed, the mixture was cooled to room temperature. The resulting solution was diluted with water (20 mL) and extracted with EtOAc (20 mL×3). The combined organic phases were washed with brine, dried over Na₂SO₄, concentrated under vacuum. The residue was purified by flash column chromatography on silica gel (PE/EtOAc=3/1) to give tert-butyl ((3-(6-(cyclopropylmethyl)-2-(6-azaspiro[2.5]octan-6-yl)nicotinamido)phenyl)(methyl)(oxo)-λ⁶-sulfaneylidene)carbamate (90 mg, 51.3%). LCMS (ESI) calcd. for C₂₉H₃₉N₄O₄S [M+H]⁺ m/z 539.27, found 539.25.

Step 5: 6-(cyclopropylmethyl)-N-(3-(S-methylsulfonimidoyl)phenyl)-2-(6-azaspiro[2.5]octan-6-yl)nicotinamide: A solution of tert-butyl ((3-(6-(cyclopropylmethyl)-2-(6-azaspiro[2.5]octan-6-yl)nicotinamido)phenyl)(methyl)(oxo)-λ⁶-sulfaneylidene)carbamate (90 mg, 0.17 mmol) in DCM (3 mL) was added TFA (0.3 mL) at room temperature. The reaction mixture was stirred at room temperature for 2 hours. After the reaction was completed, the mixture was concentrated, and the residue was adjusted to pH=8-9 with saturated aqueous NaHCO₃. The aqueous solution was extracted with DCM (30 mL×3). The combined organic phases were washed with brine, dried over sodium sulfate, concentrated under vacuum. The residue was purified by prep-HPLC (Gemini 5 um C₁₈ column, 150*21.2 mm, eluting with 30% to 85% MeCN/H₂O containing 0.1% trifluoroacetic acid) to provide 6-(cyclopropylmethyl)-N-(3-(S-methylsulfonimidoyl)phenyl)-2-(6-azaspiro[2.5]octan-6-yl)nicotinamide (45.2 mg, 60.5%) as a white solid. ¹H NMR (400 MHz, DMSO-d₆) δ 10.82 (s, 1H), 8.49 (s, 1H), 7.86 (d, J=7.9 Hz, 1H), 7.78 (d, J=7.5 Hz, 1H), 7.67-7.53 (m, 2H), 6.90 (d, J=7.7 Hz, 1H), 4.20 (s, 1H), 3.33 (s, 4H), 3.05 (s, 3H), 2.57 (d, J=7.0 Hz, 2H), 1.39 (s, 4H), 1.08 (s, 1H), 0.48 (d, J=7.7 Hz, 2H), 0.35-0.16 (m, 6H). LCMS (ESI) calcd. for C₂₄H₃₁N₄O₂S [M+H]⁺ m/z 439.22, found 439.10.

### Example 81

### 5-(4-Cyanophenyl)-2-(4,4-difluoroazepan-1-yl)-4-methyl-N-(3-(S-methylsulfonimidoyl phenyl)nicotinamide

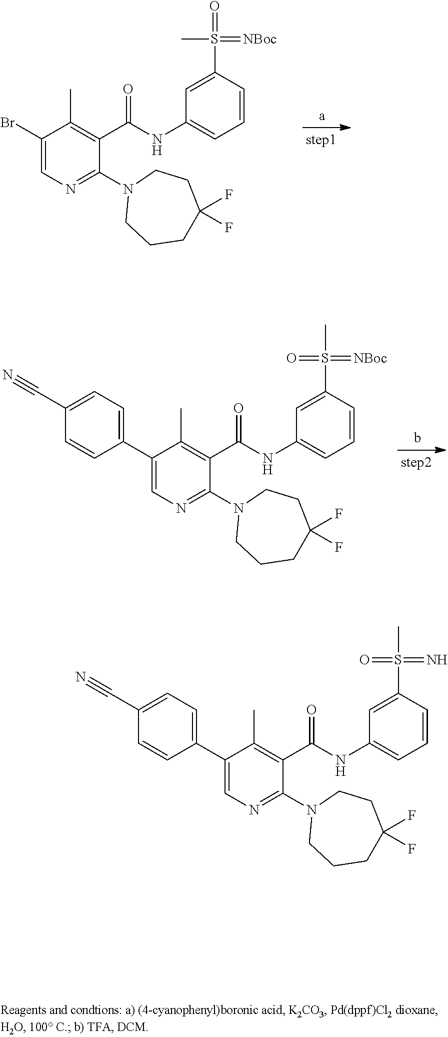

Step 1: tert-butyl ((3-(5-(4-cyanophenyl)-2-(4,4-difluoroazepan-1-yl)-4-methylnicotinamido)phenyl)(methyl)(oxo)-λ⁶-sulfaneylidene)carbamate: A mixture of tert-butyl ((3-(5-bromo-2-(4,4-difluoroazepan-1-yl)-4-methylnicotinamido)phenyl)(methyl)(oxo)-λ⁶--sulfaneylidene)carbamate (100 mg, 0.167 mmol), (4-cyanophenyl)boronic acid (38 mg, 0.250 mmol), potassium carbonate (58 mg, 0.418 mmol) and [1,1′-Bis(diphenylphosphino)ferrocene]dichloropalladium (II) (13 mg, 0.017 mmol) in 1,4-dioxane/H₂O (4/1, 10 mL) was heated at 100° C. for 6 hours under an atmosphere of N₂. After the reaction was completed, the mixture was diluted with water (10 mL) and extracted with DCM (10 mL×3). The combined organic phases were washed with brine, dried over Na₂SO₄ and concentrated under vacuum. The residue was purified by flash column chromatography on silica gel (PE/EtAOc=1/1) to give tert-butyl ((3-(5-(4-cyanophenyl)-2-(4,4-difluoroazepan-1-yl)-4-methylnicotinamido)phenyl)(methyl)(oxo)-λ⁶-sulfaneylidene)carbamate (74 mg, 71.15%) as a yellow oil. LCMS (ESI) calcd. for C₃₂H₃₆F₂N₅O₄S [M+H]⁺ m/z 624.24, found 624.20.

Step 2: 5-(4-cyanophenyl)-2-(4,4-difluoroazepan-1-yl)-4-methyl-N-(3-(S-methylsulfonimidoyl)phenyl)nicotinamide: A solution of tert-butyl ((3-(5-(4-cyanophenyl)-2-(4,4-difluoroazepan-1-yl)-4-methylnicotinamido)phenyl)(methyl)(oxo)-λ⁶-sulfaneylidene)carbamate (74 mg, 0.119 mmol) in DCM (5 mL) was added TFA (0.5 mL) at room temperature. The reaction mixture was stirred at room temperature for 2 hours. After the reaction was completed, the solution was diluted with water (10 mL) and extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.1% formic acid) to obtain 5-(4-cyanophenyl)-2-(4,4-difluoroazepan-1-yl)-4-methyl-N-(3-(S-methylsulfonimidoyl)phenyl)nicotinamide (24.5 mg, 39.52%) as a white solid. ¹H NMR (400 MHz, DMSO-d₆) δ 10.89 (s, 1H), 8.36 (s, 1H), 8.13 (s, 1H), 7.93-7.89 (m, 3H), 7.67 (d, J=7.6 Hz, 1H), 7.60-7.56 (m, 3H), 4.22 (s, 1H), 3.68-3.66 (m, 2H), 3.61-3.58 (m, 2H), 3.06 (s, 3H), 2.32-2.29 (m, 2H), 2.16 (s, 3H), 2.02-2.00 (m, 2H), 1.83-1.82 (m, 2H). LCMS (ESI) calcd. for C₂₇H₂₈F₂N₅O₂S [M+H]⁺ m/z 524.19, found 524.15.

### Example 82

### 2-(4,4-difluoroazepan-1-yl)-5-(3-fluorophenyl)-4-methyl-N-(3-(S-methylsulfonimidoyl)phenyl)nicotinamide

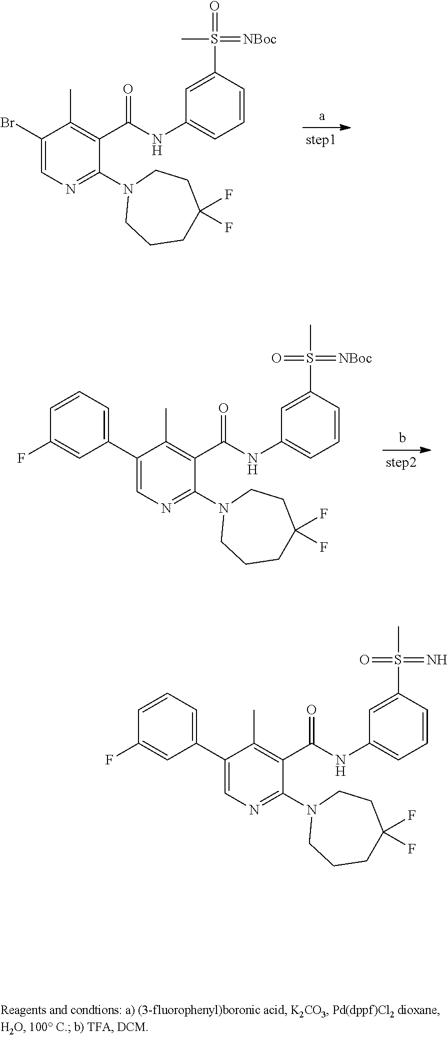

Step 1: tert-butyl ((3-(2-(4,4-difluoroazepan-1-yl)-5-(3-fluorophenyl)-4-methylnicotinamido)phenyl)(methyl)(oxo)-λ⁶-sulfaneylidene)carbamate: A mixture of tert-butyl ((3-(5-bromo-2-(4,4-difluoroazepan-1-yl)-4-methylnicotinamido)phenyl)(methyl)(oxo)-λ⁶-sulfaneylidene)carbamate (100 mg, 0.167 mmol), (3-fluorophenyl)boronic acid (35 mg, 0.250 mmol), potassium carbonate (58 mg, 0.418 mmol) and [1,1′-bis(diphenylphosphino)ferrocene]dichloropalladium (II) (13 mg, 0.017 mmol) in 1,4-dioxane/H₂O (4/1, 10 mL) was heated at 100° C. for 6 hours under an atmosphere of N₂. After the reaction was completed, the mixture was diluted with water (10 mL) and extracted with DCM (10 mL×3). The combined organic phases were washed with brine, dried over Na₂SO₄ and concentrated under vacuum. The residue was purified by flash column chromatography on silica gel (PE/EtAOc=1/1) to give tert-butyl ((3-(2-(4,4-difluoroazepan-1-yl)-5-(3-fluorophenyl)-4-methylnicotinamido)phenyl)(methyl)(oxo)-λ⁶-sulfaneylidene)carbamate (65 mg, 63.72%) as a yellow oil. LCMS (ESI) calcd. for C₃₁H₃₆F₃N₄O₄S [M+H]⁺ m/z 617.23, found 617.25.

Step 2: 2-(4,4-difluoroazepan-1-yl)-5-(3-fluorophenyl)-4-methyl-N-(3-(S-methylsulfonimidoyl)phenyl)nicotinamide: A solution of give tert-butyl ((3-(2-(4,4-difluoroazepan-1-yl)-5-(3-fluorophenyl)-4-methylnicotinamido)phenyl)(methyl)(oxo)-λ⁶-sulfaneylidene)carbamate (65 mg, 0.106 mmol) in DCM (5 mL) was added TFA (0.5 mL) at room temperature. The reaction mixture was stirred at room temperature for 2 hours. After the reaction was completed, the solution was diluted with water (10 mL) and extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 55% to 90% MeCN/H₂O containing 0.1% formic acid) to obtain 2-(4,4-difluoroazepan-1-yl)-5-(3-fluorophenyl)-4-methyl-N-(3-(S-methylsulfonimidoyl)phenyl)nicotinamide (25.1 mg, 46.29%) as a white solid. ¹H NMR (400 MHz, DMSO-d₆) δ 10.87 (s, 1H), 8.37 (s, 1H), 8.11 (s, 1H), 7.90 (d, J=8.4 Hz, 1H), 7.66 (d, J=8 Hz, 1H), 7.58-7.56 (m, 1H), 7.53-7.47 (m, 1H), 4.22 (s, 1H), 3.65-3.60 (m, 2H), 3.58-3.57 (m, 2H), 3.05 (s, 3H), 2.32-2.26 (m, 2H), 2.16 (s, 3H), 2.00-1.96 (m, 2H), 1.83-1.81 (m, 2H). LCMS (ESI) calcd. for C₂₆H₂₈F₃N₄O₂S [M+H]⁺ m/z 517.18, found 517.15.

### Example 83

### 2-(4,4-difluoroazepan-1-yl)-4-methyl-5-(1-methyl-1H-pyrazol-4-yl)-N-(3-(S-methylsulfonimidoyl)phenyl)nicotinamide

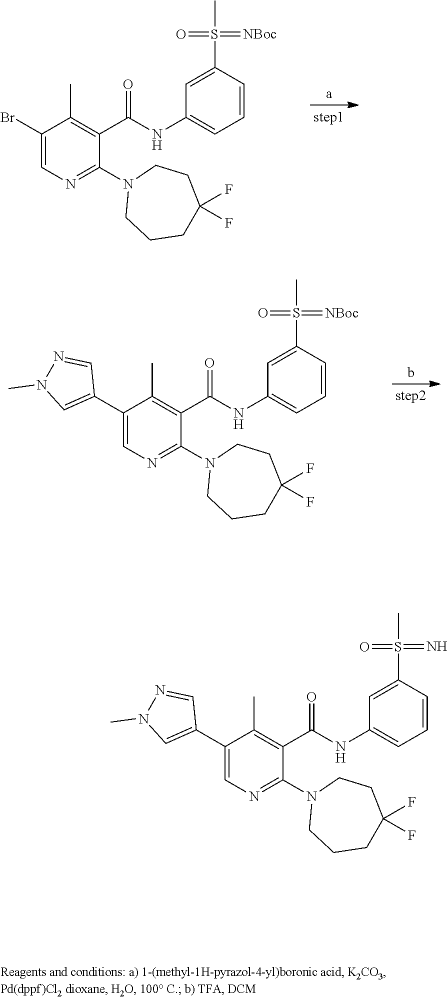

Step 1: tert-butyl ((3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)carbamate: A mixture of tert-butyl ((3-(5-bromo-2-(4,4-difluoroazepan-1-yl)-4-methylnicotinamido)phenyl)(methyl)(oxo)-λ⁶-sulfaneylidene)carbamate (100 mg, 0.166 mmol), (1-methyl-1H-pyrazol-4-yl)boronic acid (63 mg, 0.50 mmol), potassium carbonate (69.0 mg, 0.50 mmol) and [1,1′-Bis(diphenylphosphino)ferrocene]dichloropalladium (II) (12 mg, 0.016 mmol) in 1,4-dioxane/H₂O (4/1, 4 mL) was heated at 100° C. for 16 h under an atmosphere of N₂. After the reaction was completed, the mixture was diluted with water (30 mL) and extracted with EtOAc (30 mL×3). The combine organic phases were washed with brine, dried over Na₂SO₄ and concentrated under vacuum. The residue was purified by flash column chromatography on silica gel (PE/EtAOc=1/1) to give tert-butyl ((3-(2-(4,4-difluoroazepan-1-yl)-4-methyl-5-(1-methyl-1H-pyrazol-4-yl)nicotinamido)phenyl)(methyl)(oxo)-λ⁶--sulfaneylidene)carbamate (78 mg, 78%) as a white oil. LCMS (ESI) calcd. for C₂₉H₃₇F₂N₆O₄S [M+H]⁺ m/z 603.26, found 603.15.

Step 2: 2-(4,4-difluoroazepan-1-yl)-4-methyl-5-(1-methyl-1H-pyrazol-4-yl)-N-(3-(S-methylsulfonimidoyl)phenyl)nicotinamide: A solution of tert-butyl ((3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)carbamate (78 mg, 0.13 mmol) in DCM (3 mL) was added TFA (1 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 30% to 85% MeCN/H₂O containing 0.1% formic acid) to provide 2-(4,4-difluoroazepan-1-yl)-4-methyl-5-(1-methyl-1H-pyrazol-4-yl)-N-(3-(S-methylsulfonimidoyl)phenyl)nicotinamide (23.5 mg, 36%) as a white solid. ¹H NMR (400 MHz, DMSO-d₆) δ 10.85 (s, 1H), 8.37 (s, 1H), 8.17 (s, 1H), 7.89 (d, J=3.4 Hz, 2H), 7.67 (d, J=7.8 Hz, 1H), 7.63-7.55 (m, 2H), 4.22 (s, 1H), 3.88 (s, 3H), 3.65-3.49 (m, 4H), 3.06 (s, 3H), 2.27 (d, J=16.4 Hz, 5H), 1.98 (t, J=11.3 Hz, 2H), 1.80 (d, J=5.6 Hz, 2H). LCMS (ESI) calcd. for C₂₄H₂₉F₂N₆O₂S [M+H]⁺ m/z 503.21, found 503.15.

### Example 84

### 6-(4,4-difluoroazepan-1-yl)-4-methyl-N-(3-(S-methylsulfonimidoyl)phenyl)-[3,3bipyridine]-5-carboxamide

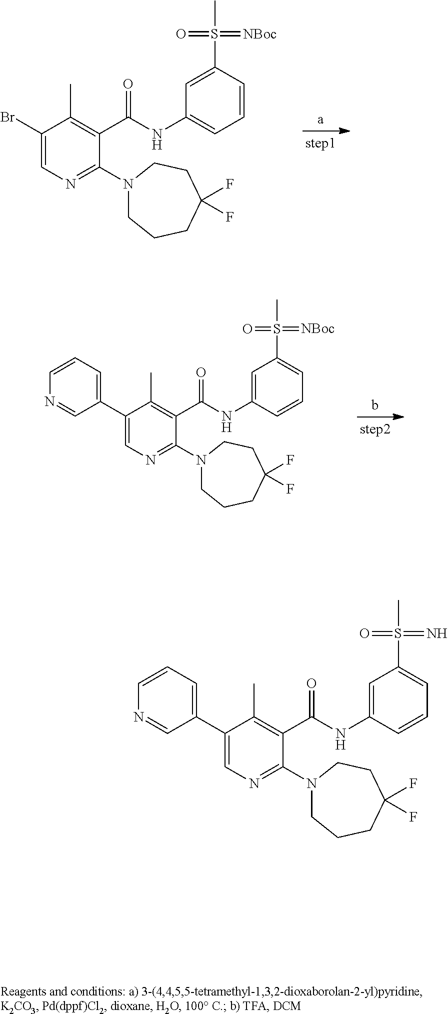

Step 1: tert-butyl ((3-(6-(4,4-difluoroazepan-1-yl)-4-methyl-[3,3 [symbol] bipyridine]-5-carboxamido)phenyl)(methyl)(oxo)-λ⁶-sulfaneylidene)carbamate: A mixture of tert-butyl ((3-(5-bromo-2-(4,4-difluoroazepan-1-yl)-4-methylnicotinamido)phenyl)(methyl)(oxo)-λ⁶-sulfaneylidene)carbamate (100 mg, 0.166 mmol), 3-(4,4,5,5-tetramethyl-1,3,2-dioxaborolan-2-yl)pyridine (102.5 mg, 0.50 mmol), potassium carbonate (69.0 mg, 0.50 mmol) and [1,1′-Bis(diphenylphosphino)ferrocene]dichloropalladium (II) (12 mg, 0.016 mmol) in 1,4-dioxane/H₂O (4/1, 4 mL) was heated at 100° C. for 16 h under an atmosphere of N₂. After the reaction was completed, the mixture was cooled to room temperature. The resulting solution was diluted with water (30 mL) and extracted with EtOAc (30 mL×3). The combine organic phases were washed with brine, dried over Na₂SO₄ and concentrated under vacuum. The residue was purified by flash column chromatography on silica gel (PE/EtAOc=1/1) to give tert-butyl ((3-(6-(4,4-difluoroazepan-1-yl)-4-methyl-[3,3 [symbol] bipyridine]-5-carboxamido)phenyl)(methyl)(oxo)-λ⁶-sulfaneylidene)carbamate (85 mg, 85.4%) as a white oil. LCMS (ESI) calcd. for C₃₀H₃₆F₂N₅O₄S [M+H]⁺ m/z 600.25, found 600.20.

Step 2: 6-(4,4-difluoroazepan-1-yl)-4-methyl-N-(3-(S-methylsulfonimidoyl)phenyl)-[3,3 [symbol] bipyridine]-5-carboxamide: A solution of tert-butyl ((3-(6-(4,4-difluoroazepan-1-yl)-4-methyl-[3,3 [symbol] bipyridine]-5-carboxamido)phenyl)(methyl)(oxo)-λ⁶-sulfaneylidene)carbamate (85 mg, 0.14 mmol) in DCM (3 mL) was added TFA (1 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 30% to 85% MeCN/H₂O containing 0.1% formic acid) to provide 6-(4,4-difluoroazepan-1-yl)-4-methyl-N-(3-(S-methylsulfonimidoyl)phenyl)-[3,3-bipyridine]-5-carboxamide (22.3 mg, 32%) as a white solid. ¹H NMR (400 MHz, DMSO-d₆) δ 10.90 (s, 1H), 8.65-8.52 (m, 2H), 8.38 (s, 1H), 8.13 (s, 1H), 7.86 (dd, J=37.2, 7.9 Hz, 2H), 7.67 (d, J=7.8 Hz, 1H), 7.62-7.47 (m, 2H), 3.70-3.58 (m, 4H), 3.07 (s, 3H), 2.30 (t, J=10.9 Hz, 2H), 2.16 (s, 3H), 2.00 (dd, J=14.7, 7.3 Hz, 2H), 1.83 (d, J=5.4 Hz, 2H). LCMS (ESI) calcd. for C₂₅H₂₈F₂N₅O₂S [M+H]⁺ m/z 500.20, found 500.10.

### Example 85

### 6-(4,4-difluoroazepan-1-yl)-4-methyl-N-(3-(S-methylsulfonimidoyl)phenyl)-[3,4bipyridine]-5-carboxamide

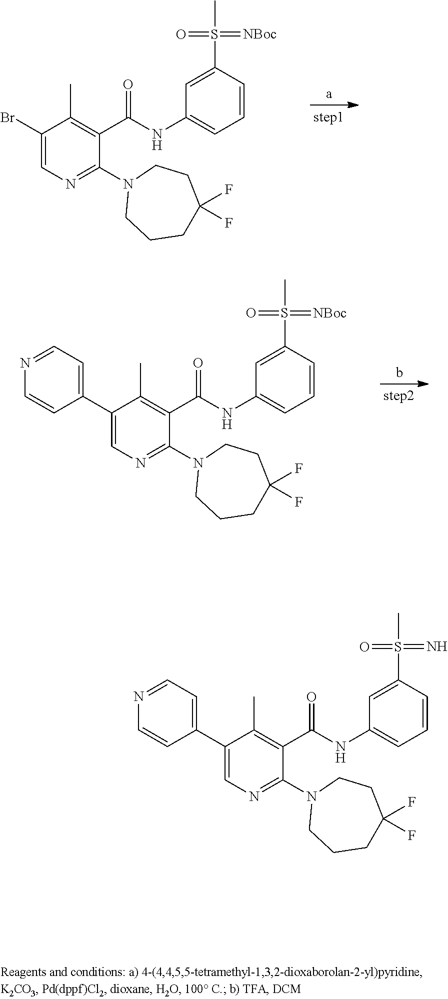

Step 1: tert-butyl ((3-(6-(4,4-difluoroazepan-1-yl)-4-methyl-[3,4 [symbol] bipyridine]-5-carboxamido)phenyl)(methyl)(oxo)-λ⁶-sulfaneylidene)carbamate: A mixture of tert-butyl ((3-(5-bromo-2-(4,4-difluoroazepan-1-yl)-4-methylnicotinamido)phenyl)(methyl)(oxo)-λ⁶-sulfaneylidene)carbamate (120 mg, 0.200 mmol), 4-(4,4,5,5-tetramethyl-1,3,2-dioxaborolan-2-yl)pyridine (61 mg, 0.300 mmol), potassium carbonate (69 mg, 0.500 mmol) and [1,1′-Bis(diphenylphosphino)ferrocene]dichloropalladium (II) (15 mg, 0.020 mmol) in 1,4-dioxane/H₂O (4/1, 10 mL) was heated at 100° C. for 6 hours under an atmosphere of N₂. After the reaction was completed, the mixture was diluted with water (10 mL) and extracted with DCM (10 mL×3). The combined organic phases were washed with brine, dried over Na₂SO₄ and concentrated under vacuum. The residue was purified by flash column chromatography on silica gel (PE/EtAOc=1/1) to give tert-butyl ((3-(6-(4,4-difluoroazepan-1-yl)-4-methyl-[3,4 [symbol] bipyridine]-5-carboxamido)phenyl)(methyl)(oxo)-λ⁶-sulfaneylidene)carbamate (90 mg, 74.89%) as a yellow oil. LCMS (ESI) calcd. for C₃₀H₃₆F₂N₅O₄S [M+H]⁺ m/z 600.24, found 600.20.

Step 2: 6-(4,4-difluoroazepan-1-yl)-4-methyl-N-(3-(S-methylsulfonimidoyl)phenyl)-[3,4 [symbol] bipyridine]-5-carboxamide: A solution of give tert-butyl ((3-(6-(4,4-difluoroazepan-1-yl)-4-methyl-[3,4 [symbol] bipyridine]-5-carboxamido)phenyl)(methyl)(oxo)-λ⁶-sulfaneylidene)carbamate (90 mg, 0.150 mmol) in DCM (5 mL) was added TFA (0.5 mL) at room temperature. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 55% to 90% MeCN/H₂O containing 0.1% formic acid) to obtain 6-(4,4-difluoroazepan-1-yl)-4-methyl-N-(3-(S-methylsulfonimidoyl)phenyl)-[3,4 [symbol] bipyridine]-5-carboxamide (39.85 mg, 53.13%) as a white solid. ¹H NMR (400 MHz, DMSO-d₆) δ 10.89 (s, 1H), 8.64 (d, J=5.2 Hz, 2H), 8.36 (s, 1H), 8.15 (s, 1H), 7.90 (d, J=8.4 Hz, 1H), 7.67 (d, J=7.6 Hz, 1H), 7.60-7.56 (m, 1H), 7.41 (d, J=5.6 Hz, 2H), 4.22 (s, 1H), 3.69-3.66 (m, 2H), 3.60-3.58 (m, 2H), 3.06 (s, 3H), 2.32-2.29 (m, 2H), 2.19 (s, 3H), 2.03-1.96 (m, 2H), 1.84-1.82 (m, 2H). LCMS (ESI) calcd. for C₂₅H₂₈F₂N₅O₂S [M+H]⁺ m/z 500.19, found 500.10.

### Example 86

### 5-bromo-2-(4,4-difluoroazepan-1-yl)-4-methyl-N-(3-(S-methylsulfonimidoyl)phenyl)nicotinamide

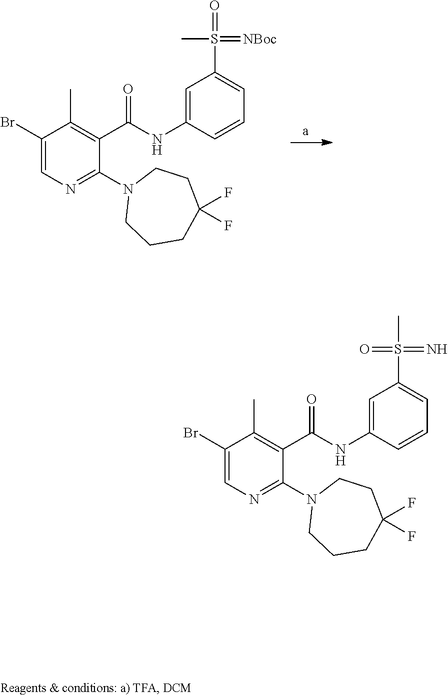

□ A solution of tert--butyl ((3-(5-bromo-2-(4,4-difluoroazepan-1-yl)-4-methylnicotinamido)phenyl)(methyl)(oxo)-λ⁶-sulfaneylidene)carbamate (60 mg, 0.1 mmol) in DCM (2 mL) was added TFA (0.5 mL) at room temperature. The solution was stirred at room temperature for 2 hours. Then the solution was concentrated and the residue was adjusted to pH=8-9 with saturated aqueous NaHCO₃. The aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 55% to 90% MeCN/H₂O containing 0.1% formic acid) to obtain 5-bromo-2-(4,4-difluoroazepan-1-yl)-4-methyl-N-(3-(S-methylsulfonimidoyl)phenyl)nicotinamide (25.7 mg, 51%) as a white solid. ¹H NMR (400 MHz, DMSO-d₆) δ 10.88 (s, 1H), 8.31 (d, J=4.2 Hz, 2H), 7.89 (d, J=8.2 Hz, 1H), 7.68 (d, J=7.9 Hz, 1H), 7.59 (t, J=7.9 Hz, 1H), 4.27 (s, 1H), 3.66-3.46 (m, 4H), 3.06 (s, 3H), 2.28 (d, J=12.4 Hz, 5H), 1.98 (s, 2H), 1.80 (d, J=5.6 Hz, 2H). LCMS (ESI) calcd. for C₂₀H₂₄BrF₂N₄O₂S [M+H]⁺ m/z 501.07, found 501.05

### Example 87

### 2-(4,4-difluoroazepan-1-yl)-5-(4-(difluoromethyl)phenyl)-4-methyl-N-(3-(S-methylsulfonimidoyl)phenyl)nicotinamide

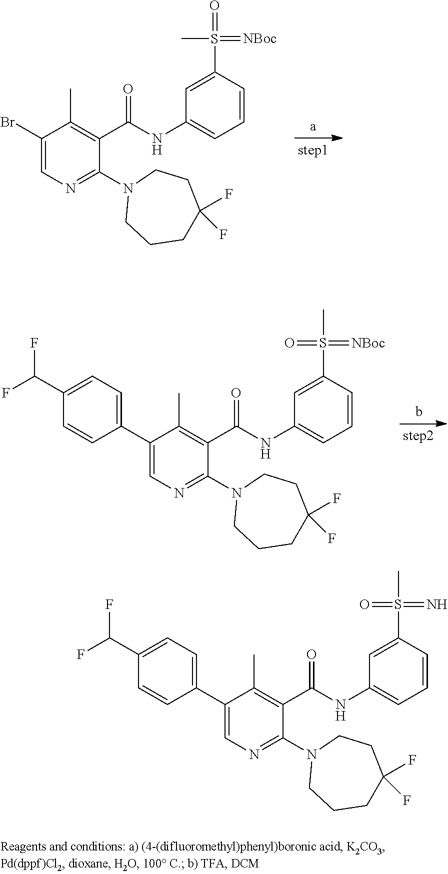

Step 1: tert-butyl ((3-(2-(4,4-difluoroazepan-1-yl)-5-(4-(difluoromethyl)phenyl)-4-methylnicotinamido)phenyl)(methyl)(oxo)-λ⁶-sulfaneylidene)carbamate: A mixture of tert-butyl ((3-(5-bromo-2-(4,4-difluoroazepan-1-yl)-4-methylnicotinamido)phenyl)(methyl)(oxo)-λ⁶-sulfaneylidene)carbamate (100 mg, 0.166 mmol), (4-(difluoromethyl)phenyl)boronic acid (43 mg, 0.249 mmol), potassium carbonate (92 mg, 0.664 mmol) and [1,1′-Bis(diphenylphosphino)ferrocene]dichloropalladium (II) (12 mg, 0.017 mmol) in 1,4-dioxane/H₂O (4/1, 10 mL) was heated at 100° C. for 6 hours under an atmosphere of N₂. After the reaction was completed, the mixture was diluted with water (10 mL) and extracted with DCM (10 mL×3). The combined organic phases were washed with brine, dried over Na₂SO₄, and concentrated under vacuum. The residue was purified by flash column chromatography on silica gel (PE/EtAOc=1/1) to give tert-butyl ((3-(2-(4,4-difluoroazepan-1-yl)-5-(4-(difluoromethyl)phenyl)-4-methylnicotinamido)phenyl)(methyl)(oxo)-λ⁶-sulfaneylidene)carbamate (80 mg, 74%) as a yellow oil. LCMS (ESI) calcd. for C₃₂H₃₇F₄N₄O₄S [M+H]⁺ m/z 649.24, found 649.20.

Step 2: 2-(4,4-difluoroazepan-1-yl)-5-(4-(difluoromethyl)phenyl)-4-methyl-N-(3-(S-methylsulfonimidoyl)phenyl)nicotinamide: A solution of tert-butyl ((3-(2-(4,4-difluoroazepan-1-yl)-5-(4-(difluoromethyl)phenyl)-4-methylnicotinamido)phenyl)(methyl)(oxo)-λ⁶-sulfaneylidene)carbamate (80 mg, 0.123 mmol) in DCM (5 mL) was added TFA (0.5 mL) at room temperature. The solution was stirred at room temperature for 2 hours then concentrated under vacuum. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 55% to 90% MeCN/H₂O containing 0.1% formic acid) to obtain 2-(4,4-difluoroazepan-1-yl)-5-(4-(difluoromethyl)phenyl)-4-methyl-N-(3-(S-methylsulfonimidoyl)phenyl)nicotinamide (28 mg, 41%) as a white solid. ¹H NMR (400 MHz, DMSO-d₆) δ 10.89 (s, 1H), 8.37 (s, 1H), 8.11 (s, 1H), 7.91 (d, J=8.4 Hz, 1H), 7.66 (d, J=7.9 Hz, 3H), 7.59 (d, J=7.9 Hz, 1H), 7.50 (d, J=7.9 Hz, 2H), 7.10 (s, 1H), 4.22 (s, 1H), 3.66 (s, 2H), 3.59 (t, J=6.0 Hz, 2H), 3.06 (s, 3H), 2.31 (d, J=13.6 Hz, 2H), 2.16 (s, 3H), 2.01 (s, 2H), 1.82 (s, 2H). LCMS (ESI) calcd. for C₂₇H₂₉F₄N₄O₂S [M+H]⁺ m/z 549.19, found 549.15.

### Example 88

### 2-(4,4-difluoroazepan-1-yl)-5-(4-fluorophenyl)-4-methyl-N-(3-(S-methylsulfonimidoyl)phenyl)nicotinamide

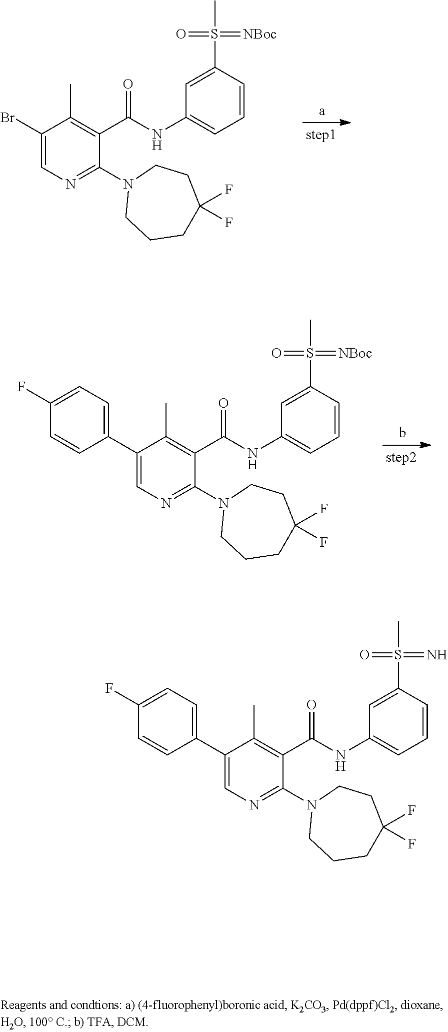

Step 1: tert-butyl ((3-(2-(4,4-difluoroazepan-1-yl)-5-(4-fluorophenyl)-4-methylnicotinamido)phenyl)(methyl)(oxo)-λ⁶-sulfaneylidene)carbamate: A mixture of tert-butyl ((3-(5-bromo-2-(4,4-difluoroazepan-1-yl)-4-methylnicotinamido)phenyl)(methyl)(oxo)-λ⁶-sulfaneylidene)carbamate (100 mg, 0.167 mmol), (4-fluorophenyl)boronic acid (35 mg, 0.250 mmol), potassium carbonate (57 mg, 0.417 mmol) and [1,1′-Bis(diphenylphosphino)ferrocene]dichloropalladium (II) (12 mg, 0.017 mmol) in 1,4-dioxane/H₂O (4/1, 10 mL) was heated at 100° C. for 16 hours under an atmosphere of N₂. After the reaction was completed, the mixture was concentrated under vacuum. The residue was directly purified by flash column chromatography on silica gel (PE/EtAOc=1/1) to give tert-butyl ((3-(2-(4,4-difluoroazepan-1-yl)-5-(4-fluorophenyl)-4-methylnicotinamido)phenyl)(methyl)(oxo)-λ⁶-sulfaneylidene)carbamate (70 mg, 67.96%) as a yellow solid. LCMS (ESI) calcd. for C₃₁H₃₆F₃N₄O₄S [M+H]⁺ m/z 617.24, found 617.20.

Step 2: 2-(4,4-difluoroazepan-1-yl)-5-(4-fluorophenyl)-4-methyl-N-(3-(S-methylsulfonimidoyl)phenyl)nicotinamide: A solution of tert-butyl ((3-(2-(4,4-difluoroazepan-1-yl)-5-(4-fluorophenyl)-4-methylnicotinamido)phenyl)(methyl)(oxo)-λ⁶-sulfaneylidene)carbamate (70 mg, 0.113 mmol) in DCM (5 mL) was added TFA (0.5 mL) at room temperature. The solution was stirred at room temperature for 2 hours then concentrated under vacuum. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 55% to 90% MeCN/H₂O containing 0.1% formic acid) to obtain 2-(4,4-difluoroazepan-1-yl)-5-(4-fluorophenyl)-4-methyl-N-(3-(S-methylsulfonimidoyl)phenyl)nicotinamide (38 mg, 64.85%) as a white solid. ¹H NMR (400 MHz, DMSO-d₆) δ 10.87 (s, 1H), 8.37 (s, 1H), 8.07 (s, 1H), 7.90 (d, J=8.1 Hz, 1H), 7.70-7.54 (m, 2H), 7.42-7.25 (m, 4H), 4.22 (s, 1H), 3.71-3.49 (m, 4H), 3.06 (s, 3H), 2.35-2.22 (m, 2H), 2.14 (s, 3H), 2.07-1.95 (m, 2H), 1.86-1.76 (m, 2H). LCMS (ESI) calcd. for C₂₆H₂₈F₃N₄O₂S [M+H]⁺ m/z 517.19, found 517.15.

### Example 89

### 2-(4,4-difluoroazepan-1-yl)-4-methyl-5-(1-methyl-1H-pyrazol-3-yl)-N-(3-(S-methylsulfonimidoyl)phenyl)nicotinamide

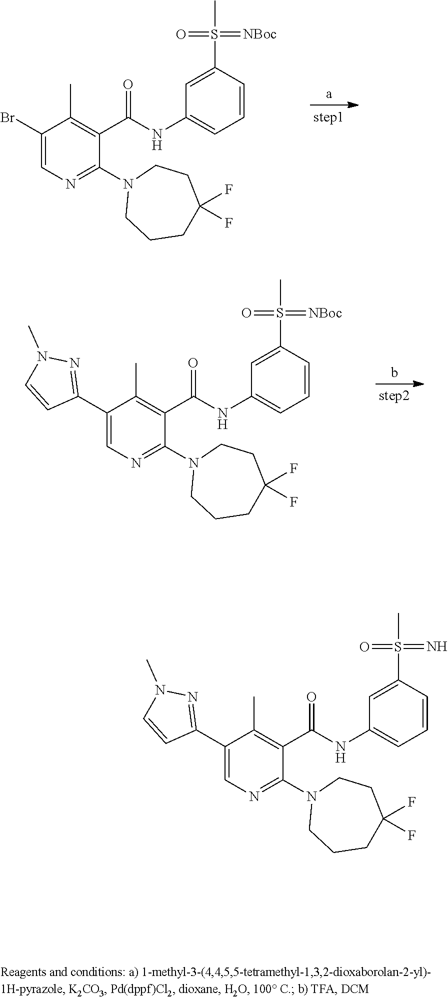

Step 1: tert-butyl ((3-(2-(4,4-difluoroazepan-1-yl)-4-methyl-5-(1-methyl-1H-pyrazol-3-yl)nicotinamido)phenyl)(methyl)(oxo)-λ⁶-sulfaneylidene)carbamate: A mixture of tert-butyl ((3-(5-bromo-2-(4,4-difluoroazepan-1-yl)-4-methylnicotinamido)phenyl)(methyl)(oxo)-λ⁶-sulfaneylidene)carbamate (100 mg, 0.167 mmol), 1-methyl-3-(4,4,5,5-tetramethyl-1,3,2-dioxaborolan-2-yl)-1H-pyrazole (52 mg, 0.250 mmol), potassium carbonate (57 mg, 0.417 mmol) and [1,1′-Bis(diphenylphosphino)ferrocene]dichloropalladium (II) (12 mg, 0.017 mmol) in 1,4-dioxane/H₂O (4/1, 10 mL) was heated at 100° C. for 16 hours under an atmosphere of N₂.

After the reaction was completed, the mixture was concentrated under vacuum. The residue was purified by flash column chromatography on silica gel (PE/EtAOc=1/1) to give tert-butyl ((3-(2-(4,4-difluoroazepan-1-yl)-4-methyl-5-(1-methyl-1H-pyrazol-3-yl)nicotinamido)phenyl)(methyl)(oxo)-λ⁶-sulfaneylidene)carbamate (80 mg, 79.76%) as a yellow solid. LCMS (ESI) calcd. for C₂₉H₃₇F₂N₆O₄S [M+H]⁺ m/z 603.26, found 603.30.

Step 2: 2-(4,4-difluoroazepan-1-yl)-4-methyl-5-(1-methyl-1H-pyrazol-3-yl)-N-(3-(S-methylsulfonimidoyl)phenyl)nicotinamide: A solution of tert-butyl ((3-(2-(4,4-difluoroazepan-1-yl)-4-methyl-5-(1-methyl-1H-pyrazol-3-yl)nicotinamido)phenyl)(methyl)(oxo)-λ⁶-sulfaneylidene)carbamate (80 mg, 0.133 mmol) in DCM (5 mL) was added TFA (0.5 mL) at room temperature. The solution was stirred at room temperature for 2 hours then concentrated under vacuum. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 55% to 90% MeCN/H₂O containing 0.1% formic acid) to obtain 2-(4,4-difluoroazepan-1-yl)-4-methyl-5-(1-methyl-1H-pyrazol-3-yl)-N-(3-(S-methylsulfonimidoyl)phenyl)nicotinamide (40 mg, 59.97%) as a white solid. ¹H NMR (400 MHz, DMSO-d₆) δ 10.86 (s, 1H), 8.37 (s, 1H), 8.29 (s, 1H), 7.91 (d, J=8.0 Hz, 1H), 7.76 (d, J=2.0 Hz, 1H), 7.68-7.55 (m, 2H), 6.44 (d, J=2.1 Hz, 1H), 4.22 (s, 1H), 3.89 (s, 3H), 3.66-3.52 (m, 4H), 3.06 (s, 3H), 2.38-2.19 (m, 5H), 2.07-1.95 (m, 2H), 1.81 (d, J=5.5 Hz, 2H). LCMS (ESI) calcd. for C₂₄H₂₉F₂N₆O₂S [M+H]⁺ m/z 503.20, found 503.10.

### Example 90

### (S)-2-(4,4-difluoroazepan-1-yl)-4-methyl-N-(3-(S-methylsulfonimidoyl)phenyl)-5-(2-oxa-6-azaspiro[3.3]heptan-6-yl)nicotinamide

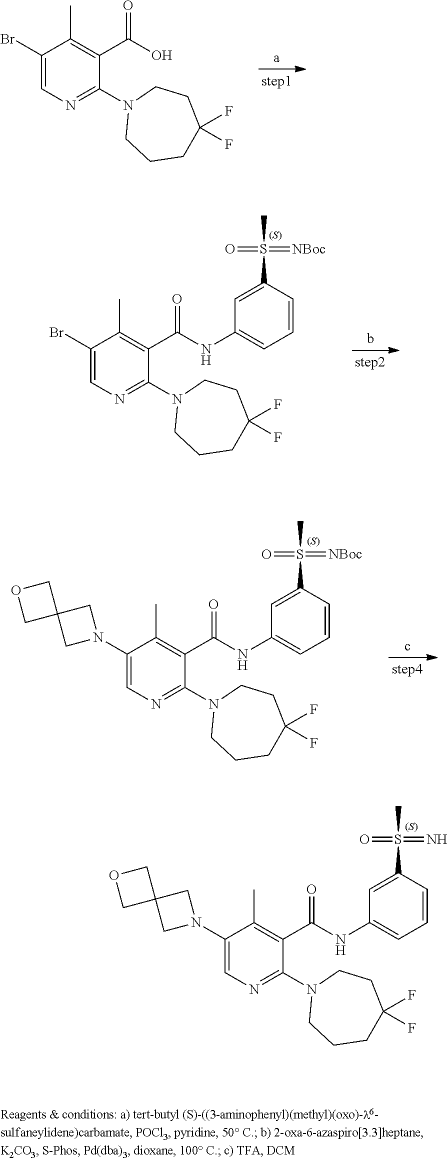

Step 1: tert-butyl (S)-((3-(5-bromo-2-(4,4-difluoroazepan-1-yl)-4-methylnicotinamido)phenyl)(methyl)(oxo)-λ⁶-sulfaneylidene)carbamate: A mixture of 5-bromo-2-(4,4-difluoroazepan-1-yl)-4-methylnicotinic acid (200 mg, 0.57 mmol) and tert-butyl (S)-((3-aminophenyl)(methyl)(oxo)-λ⁶-sulfaneylidene)carbamate (309 mg, 1.15 mmol) in pyridine (4 mL) was added POCl₃ (150 L) dropwise at 50° C. The reaction was stirred at 50° C. for 1 hour. After the reaction was completed, the resulting solution was quenched with water (20 mL) and extracted with DCM (20 mL×3). The combined organic phases were washed with brine, dried over sodium sulfate and concentrated under vacuum. The residue was purified by flash column chromatography on silica gel (PE/EtOAc=2/1) to give tert-butyl (S)-((3-(5-bromo-2-(4,4-difluoroazepan-1-yl)-4-methylnicotinamido)phenyl)(methyl)(oxo)-λ⁶-sulfaneylidene)carbamate (100 mg, 28.99%) as a yellow solid. LCMS (ESI) calcd. for C₂₅H₃₂BrF₂N₄O₄S [M+H]⁺ m/z 603.13, found 603.10.

Step 2: tert-butyl (S)-((3-(2-(4,4-difluoroazepan-1-yl)-4-methyl-5-(2-oxa-6-azaspiro[3.3]heptan-6-yl)nicotinamido)phenyl)(methyl)(oxo)-λ⁶-sulfaneylidene)carbamate: A mixture of tert-butyl (S)-((3-(5-bromo-2-(4,4-difluoroazepan-1-yl)-4-methylnicotinamido)phenyl)(methyl)(oxo)-λ⁶-sulfaneylidene)carbamate (100 mg, 0.167 mmol), 2-oxa-6-azaspiro[3.3]heptane (25 mg, 0.250 mmol), potassium carbonate (57 mg, 0.417 mmol), S-Phos (7 mg, 0.017 mmol) and Pd(dba)₃ (10 mg, 0.017 mmol) in 1,4-dioxane (10 mL) was heated at 100° C. for 16 hours under an atmosphere of N₂. After the reaction was completed, the mixture was concentrated under vacuum. The residue was purified by flash column chromatography on silica gel (PE/EtAOc=1/1) to give tert-butyl (S)-((3-(2-(4,4-difluoroazepan-1-yl)-4-methyl-5-(2-oxa-6-azaspiro[3.3]heptan-6-yl)nicotinamido)phenyl)(methyl)(oxo)-λ⁶-sulfaneylidene)carbamate (90 mg, 87.38%) as a yellow oil. LCMS (ESI) calcd. for C₃₀H₄₀F2N₅O₅S [M+H]⁺ m/z 620.27, found 620.25.

Step 3: (S)-2-(4,4-difluoroazepan-1-yl)-4-methyl-N-(3-(S-methylsulfonimidoyl)phenyl)-5-(2-oxa-6-azaspiro[3.3]heptan-6-yl)nicotinamide: A solution of tert-butyl (S)-((3-(2-(4,4-difluoroazepan-1-yl)-4-methyl-5-(2-oxa-6-azaspiro[3.3]heptan-6-yl)nicotinamido)phenyl)(methyl)(oxo)-λ⁶-sulfaneylidene)carbamate (90 mg, 0.145 mmol) in DCM (5 mL) was added TFA (0.5 mL) at room temperature. The mixture was stirred at room temperature for 2 h hours then concentrated under vacuum. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 55% to 90% MeCN/H₂O containing 0.1% formic acid) to obtain (S)-2-(4,4-difluoroazepan-1-yl)-4-methyl-N-(3-(S-methylsulfonimidoyl)phenyl)-5-(2-oxa-6-azaspiro[3.3]heptan-6-yl)nicotinamide (43 mg, 56.97%) as a white solid. ¹H NMR (400 MHz, DMSO-d₆) δ 10.72 (s, 1H), 8.37 (s, 1H), 7.90 (d, J=7.9 Hz, 1H), 7.66 (d, J=7.8 Hz, 1H), 7.62-7.53 (m, 2H), 4.72 (s, 4H), 4.21 (s, 1H), 4.00 (s, 4H), 3.42-3.36 (m, 4H), 3.05 (s, 3H), 2.23-2.11 (m, 2H), 2.07 (s, 3H), 2.03-1.91 (m, 2H), 1.77-1.66 (m, 2H). LCMS (ESI) calcd. for C₂₅H₃₂F₂N₅O₃S [M+H]⁺ m/z 520.22, found 520.20.

### Example 91

### 5-(3-cyanophenyl)-2-(4,4-difluoroazepan-1-yl)-4-methyl-N-(3-(S-methylsulfonimidoyl)phenyl)nicotinamide

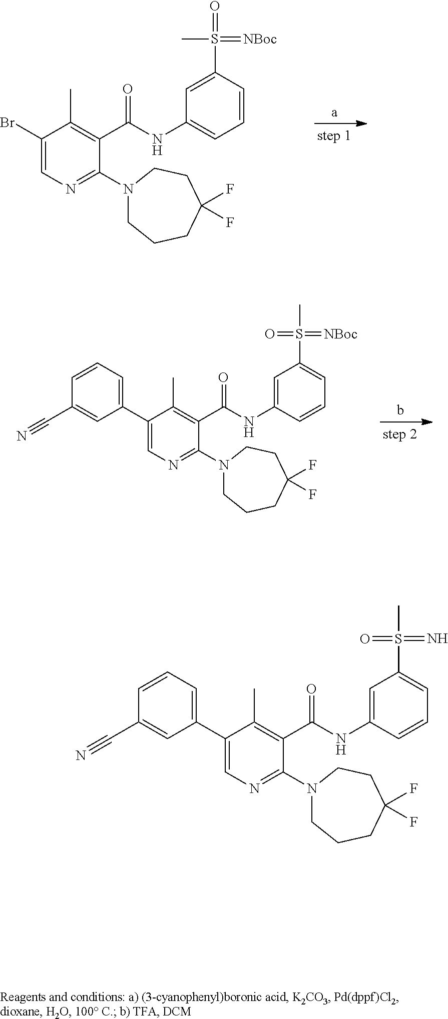

Step 1: tert-butyl ((3-(5-(3-cyanophenyl)-2-(4,4-difluoroazepan-1-yl)-4-methylnicotinamido)phenyl)(methyl)(oxo)-λ⁶-sulfaneylidene)carbamate: A mixture of tert-butyl ((3-(5-bromo-2-(4,4-difluoroazepan-1-yl)-4-methylnicotinamido)phenyl)(methyl)(oxo)-λ⁶-sulfaneylidene)carbamate (100 mg, 0.166 mmol), (3-cyanophenyl)boronic acid (37 mg, 0.249 mmol), potassium carbonate (92 mg, 0.664 mmol) and [1,1′-Bis(diphenylphosphino)ferrocene]dichloropalladium (II) (12 mg, 0.017 mmol) in 1,4-dioxane/H₂O (4/1, 10 mL) was heated at 100° C. for 6 hours under an atmosphere of N₂. After the reaction was completed, the mixture was diluted with water (20 mL) and extracted with DCM (20 mL×3). The combined organic phases were washed with brine, dried over Na₂SO₄, and concentrated under vacuum. The residue was purified by flash column chromatography on silica gel (PE/EtAOc=1/1) to give tert-butyl ((3-(5-(3-cyanophenyl)-2-(4,4-difluoroazepan-1-yl)-4-methylnicotinamido)phenyl)(methyl)(oxo)-λ⁶-sulfaneylidene)carbamate (80 mg, 77%) as a yellow oil. LCMS (ESI) calcd. for C₃₂H₃₆F₂N₅O₄S [M+H]⁺ m/z 624.24, found 624.20.

Step 2: 5-(3-cyanophenyl)-2-(4,4-difluoroazepan-1-yl)-4-methyl-N-(3-(S-methylsulfonimidoyl) phenyl) nicotinamide: A solution of tert-butyl ((3-(5-(3-cyanophenyl)-2-(4,4-difluoroazepan-1-yl)-4-methylnicotinamido)phenyl)(methyl)(oxo)-λ⁶--sulfaneylidene)carbamate (80 mg, 0.128 mmol) in DCM (5 mL) was added TFA (0.5 mL) at room temperature. The solution was stirred at room temperature for 2 hours then concentrated under vacuum. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 55% to 90% MeCN/H₂O containing 0.1% formic acid) to obtain 5-(3-cyanophenyl)-2-(4,4-difluoroazepan-1-yl)-4-methyl-N-(3-(S-methylsulfonimidoyl)phenyl)nicotinamide (35 mg, 52%) as a white solid. ¹H NMR (400 MHz, DMSO-d₆) δ 10.89 (s, 1H), 8.37 (s, 1H), 8.12 (s, 1H), 7.95-7.77 (m, 3H), 7.75-7.63 (m, 3H), 7.59 (t, J=7.9 Hz, 1H), 4.31 (s, 1H), 3.67 (s, 2H), 3.60 (t, J=6.1 Hz, 2H), 3.07 (s, 3H), 2.31 (d, J=12.2 Hz, 2H), 2.16 (s, 3H), 2.00 (s, 2H), 1.83 (d, J=5.5 Hz, 2H). LCMS (ESI) calcd. for C₂₇H₂₈F₂N₅O₂S [M+H]⁺ m/z 524.19, found 524.20.

### Example 92

### 2-(4,4-difluoroazepan-1-yl)-5-(2-fluorophenyl)-4-methyl-N-(3-(S-methylsulfonimidoyl)phenyl)nicotinamide

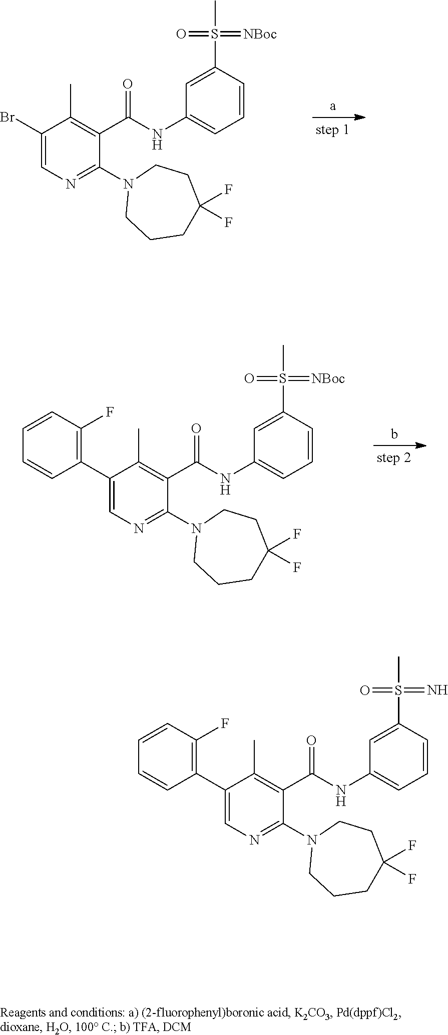

Step 1: tert-butyl ((3-(2-(4,4-difluoroazepan-1-yl)-5-(2-fluorophenyl)-4-methylnicotinamido)phenyl)(methyl)(oxo)-λ⁶--sulfaneylidene)carbamate: A mixture tert-butyl ((3-(5-bromo-2-(4,4-difluoroazepan-1-yl)-4-methylnicotinamido)phenyl)(methyl)(oxo)-λ⁶-sulfaneylidene)carbamate (100 mg, 0.16 mmol), (2-fluorophenyl)boronic acid (35 mg, 0.25 mmol), K₂CO₃ (23 mg, 0.16 mmol) and Pd(dppf)Cl₂ (12 mg, 0.02 mmol) in dioxane/H₂O (10/1, 11 mL) was heated at 100° C. for 16 h under an atmosphere of N₂. After the reaction was completed, the mixture was cooled to room temperature. The resulting solution was diluted with water (20 mL) and extracted with EtOAc (20 mL×3). The combined organic phases were washed with brine, dried over Na₂SO₄, concentrated under vacuum. The residue was purified by flash column chromatography on silica gel (PE/EtOAc=3/1) to give tert-butyl ((3-(2-(4,4-difluoroazepan-1-yl)-5-(2-fluorophenyl)-4-methylnicotinamido)phenyl)(methyl)(oxo)-λ⁶-sulfaneylidene)carbamate (60 mg, 51.3%). LCMS (ESI) calcd. for C₃₁H₃₆F₃N₄O₄S [M+H]⁺ m/z 617.24, found 617.30.

Step 2: Preparation of 2-(4,4-difluoroazepan-1-yl)-5-(2-fluorophenyl)-4-methyl-N-(3-(S-methylsulfonimidoyl)phenyl)nicotinamide: A solution of tert-butyl ((3-(2-(4,4-difluoroazepan-1-yl)-5-(2-fluorophenyl)-4-methylnicotinamido)phenyl)(methyl)(oxo)-λ⁶--sulfaneylidene)carbamate (60 mg, 0.10 mmol) in DCM (3 mL) was added TFA (0.3 mL) at room temperature. The reaction mixture was stirred at room temperature for 2 hours. After the reaction was completed, the mixture was concentrated, and the residue was adjusted to pH=8-9 with saturated aqueous NaHCO₃. The aqueous solution was extracted with DCM (30 mL×3). The combined organic phases were washed with brine, dried over sodium sulfate, concentrated under vacuum. The residue was purified by prep-HPLC (Gemini 5 um C₁₈ column, 150*21.2 mm, eluting with 35% to 85% MeCN/H₂O containing 0.1% trifluoroacetic acid) to provide 2-(4,4-difluoroazepan-1-yl)-5-(2-fluorophenyl)-4-methyl-N-(3-(S-methylsulfonimidoyl)phenyl)nicotinamide (19 mg, 37%) as a white solid. ¹H NMR (400 MHz, DMSO-d₆) δ 10.90 (s, 1H), 8.38 (s, 1H), 8.06 (s, 1H), 7.91 (d, J=7.8 Hz, 1H), 7.67 (d, J=8.0 Hz, 1H), 7.58 (t, J=7.9 Hz, 1H), 7.51-7.43 (m, 1H), 7.36-7.28 (m, 3H), 4.21 (s, 1H), 3.67 (s, 2H), 3.59 (t, J=6.1 Hz, 2H), 3.06 (s, 3H), 2.31 (d, J=13.4 Hz, 2H), 2.06 (s, 3H), 2.01 (s, 2H), 1.83 (d, J=5.6 Hz, 2H). LCMS (ESI) calcd. for C₂₆H₂₈F₃N₄O₂S [M+H]⁺ m/z 517.19, found 517.10.

### Example 93

### 2-(4,4-difluoroazepan-1-yl)-5-(4-methoxyphenyl)-4-methyl-N-(3-(S-methylsulfonimidoyl)phenyl)nicotinamide

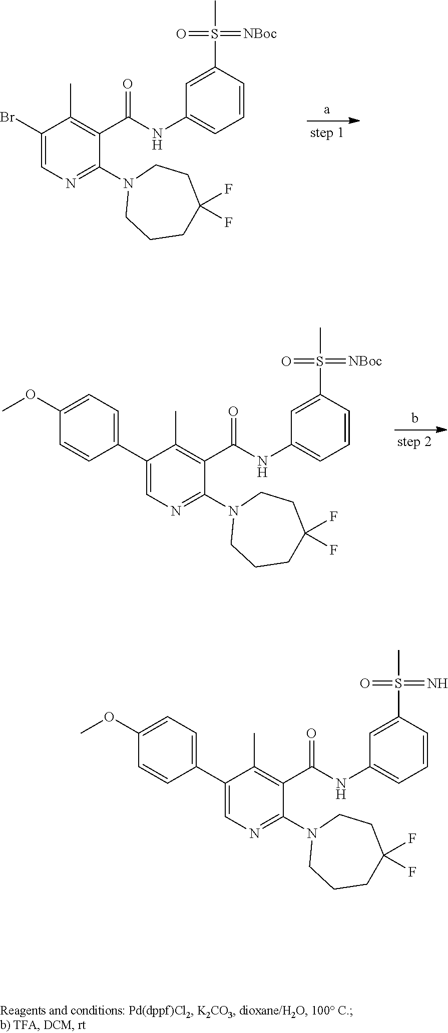

Step 1: tert-butyl ((3-(2-(4,4-difluoroazepan-1-yl)-5-(4-methoxyphenyl)-4-methylnicotinamido)phenyl)(methyl)(oxo)-λ⁶-sulfaneylidene)carbamate: A solution of tert-butyl ((3-(5-bromo-2-(4,4-difluoroazepan-1-yl)-4-methylnicotinamido)phenyl)(methyl)(oxo)-λ⁶-sulfaneylidene)carbamate (100 mg, 0.17 mmol), (4-methoxyphenyl)boronic acid (39 mg, 0.26 mmol), K₂CO₃ (69 mg, 0.50 mmol) and PdCl₂(dppf) (12 mg, 0.02 mmol) in dioxane/H₂O (10/1, 11 mL) was heated at 100° C. for 16 h under an atmosphere of N₂. The resulting solution was diluted with water (20 mL) and extracted with EtOAc (20 mL×3). The combined organic phases were washed with brine, dried over Na₂SO₄, and concentrated under vacuum. The residue was purified by flash column chromatography on silica gel (PE/EtOAc=3/1) to give tert-butyl ((3-(2-(4,4-difluoroazepan-1-yl)-5-(4-methoxyphenyl)-4-methylnicotinamido)phenyl)(methyl)(oxo)-λ⁶-sulfaneylidene)carbamate (80 mg, 53.8%) as a yellow solid. LCMS (ESI) calcd. for C₃₂H₃₉F₂N₄O₅S [M+H]⁺ m/z 629.26, found 629.20.

Step 2: 2-(4,4-difluoroazepan-1-yl)-5-(4-methoxyphenyl)-4-methyl-N-(3-(S-methylsulfonimidoyl)phenyl)nicotinamide: A solution of tert-butyl ((3-(2-(4,4-difluoroazepan-1-yl)-5-(4-methoxyphenyl)-4-methylnicotinamido)phenyl)(methyl)(oxo)-λ⁶-sulfaneylidene)carbamate (80 mg, 0.13 mmol) in DCM (1.5 mL) was added TFA (0.5 mL) at room temperature. The reaction mixture was stirred at room temperature for 2 hours. After the reaction was completed, the mixture was concentrated. The residue was dissolved in THE (2 mL) then adjusted to pH=8-9 with saturated aqueous NaHCO₃. The resulting solution was extracted with DCM (10 mL×3). The combined organic phases were washed with brine, dried over sodium sulfate and concentrated under vacuum. The residue was purified by prep-HPLC (Gemini 5 um C₁₈ column, 150*21.2 mm, eluting with 40% to 95% MeCN/H₂O containing 0.05% NH₄OH) to give 2-(4,4-difluoroazepan-1-yl)-5-(4-methoxyphenyl)-4-methyl-N-(3-(S-methylsulfonimidoyl)phenyl)nicotinamide (20 mg, 29%) as a white solid. ¹H NMR (400 MHz, DMSO-d₆) δ 10.86 (s, 1H), 8.38 (s, 1H), 8.05 (s, 1H), 7.90 (d, J=8.3 Hz, 1H), 7.68-7.55 (m, 2H), 7.26 (d, J=8.6 Hz, 2H), 7.02 (d, J=8.6 Hz, 2H), 4.21 (s, 1H), 3.80 (s, 3H), 3.63 (s, 2H), 3.57 (t, J=6.1 Hz, 2H), 3.05 (s, 3H), 2.28 (s, 2H), 2.15 (s, 3H), 2.00 (s, 2H), 1.81 (s, 2H). LCMS (ESI) calcd. for C₂₇H₃₁F₂N₄O₃S [M+H]⁺ m/z 529.21, found 529.15.

### Example 94

### 5-methyl-N-(3-(S-methylsulfonimidoyl)phenyl)-3-(6-azaspiro[2.5]octan-6-yl)-6-(trifluoromethyl)pyridazine-4-carboxamide

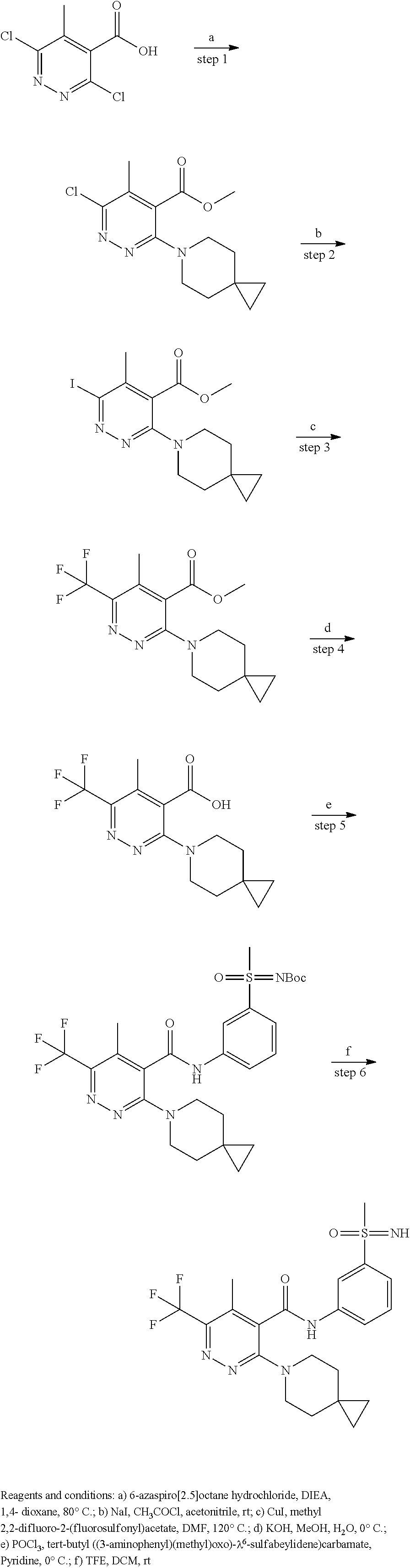

Step 1: methyl 6-chloro-5-methyl-3-(6-azaspiro[2.5]octan-6-yl)pyridazine-4-carboxylate: To a solution of methyl 3,6-dichloro-5-methylpyridazine-4-carboxylate (1.0 g, 4.52 mmol) and 6-azaspiro[2.5]octane hydrochloride (0.68 g, 4.6 mmol) in dioxane (20 mL) was added DIEA (2.91 g, 22.62 mmol). The mixture was heated at 80° C. for 2 hours. LCMS showed the reaction was completed. Then the mixture was diluted with water (50 mL) and extracted with EtOAc (70 mL×3). The combined organic phases were washed with brine, dried over sodium sulfate, concentrated under vacuum. The residue was purified by flash column chromatography on silica gel (PE/EtOAc=10/1 to 2/1) to provide methyl 6-chloro-5-methyl-3-(6-azaspiro[2.5]octan-6-yl)pyridazine-4-carboxylate (0.5 g, 31.11%) as a white solid. LCMS (ESI) calcd. for C₁₄H₁₉ClN₃O₂ [M+H]⁺ m/z 296.11, found 296.00.

Step 2: methyl 6-iodo-5-methyl-3-(6-azaspiro[2.5]octan-6-yl)pyridazine-4-carboxylate: To a mixture of NaI (2.02 g, 13.52 mmol) in MeCN (10 mL) was added CH₃COCl(0.40 g, 5.07 mmol) in MeCN (5 mL). Then a solution of methyl 6-chloro-5-methyl-3-(6-azaspiro[2.5]octan-6-yl)pyridazine-4-carboxylate (0.5 g, 1.69 mmol) in MeCN (10 mL) was added dropwise to the mixture at room temperature. The mixture was stirred at room temperature for 0.5 h. LCMS showed the reaction was completed. The mixture was quenched with water (50 mL) and extracted with EtOAc (50 mL×3). The combined organic phases were washed with brine, dried over sodium sulfate, concentrated under vacuum. The residue was purified by flash column chromatography on silica gel (PE/EtOAc=2/1) to provide methyl 6-iodo-5-methyl-3-(6-azaspiro[2.5]octan-6-yl)pyridazine-4-carboxylate (370 mg, 62.04%) as a yellow oil. LCMS (ESI) calcd. for C₁₄H₁₉IN₃O₂ [M+H]⁺ m/z 388.05, found 387.90.

Step 3: methyl 5-methyl-3-(6-azaspiro[2.5]octan-6-yl)-6-(trifluoromethyl)pyridazine-4-carboxylate: A solution of methyl 6-iodo-5-methyl-3-(6-azaspiro [2.5]octan-6-yl)pyridazine-4-carboxylate (350 mg, 0.90 mmol), CuI (343 mg, 1.81 mmol) and methyl 2,2-difluoro-2-(fluorosulfonyl)acetate (1.38 g, 7.2 mmol) in DMF (5 mL) was heated at 120° C. for 2 hours. LCMS showed the reaction was completed. Then the mixture was filtered through celite. The filtrate was diluted with water (30 mL) and extracted with EtOAc (30 mL×3). The combined organic phases were washed with brine, dried over sodium sulfate, concentrated under vacuum. The residue was purified by flash column chromatography on silica gel (PE/EtOAc=4/1) to provide methyl 5-methyl-3-(6-azaspiro [2.5]octan-6-yl)-6-(trifluoromethyl) pyridazine-4-carboxylate (200 mg, 52.94%) as a yellow solid. LCMS (ESI) calcd. for C₁₅H₁₉F₃N₃O₂ [M+H]⁺ m/z 330.14, found 330.00.

Step 4: 5-methyl-3-(6-azaspiro[2.5]octan-6-yl)-6-(trifluoromethyl)pyridazine-4-carboxylic acid: To a solution of methyl 5-methyl-3-(6-azaspiro[2.5]octan-6-yl)-6-(trifluoromethyl)pyridazine-4-carboxylate (200 mg, 0.61 mmol) in MeOH/H₂O (1/1, 4 mL) was added KOH (272 mg, 4.86 mmol) at 0° C. The mixture was stirred at 0° C. for 1 hours. After the reaction was completed, the mixture was concentrated to remove most MeOH. The aqueous phase was adjusted to pH=3-4 with 1N HCl then extracted with EtOAc (20 mL×3). The combined organic phases were washed with brine, dried with Na₂SO₄, and concentrated under reduced pressure to provide crude 5-methyl-3-(6-azaspiro[2.5]octan-6-yl)-6-(trifluoromethyl)pyridazine-4-carboxylic acid (200 mg) as a yellow solid which was used in next step without further purification. LCMS (ESI) calcd. for C₁₄H₁₇F₃N₃O₂ [M+H]⁺ m/z 316.13, found 315.95.

Step 5: tert-butyl (methyl(3-(5-methyl-3-(6-azaspiro[2.5]octan-6-yl)-6-(trifluoromethyl)pyridazine-4-carboxamido)phenyl)(oxo)-λ⁶-sulfaneylidene)carbamate: To a solution of 5-methyl-3-(6-azaspiro[2.5]octan-6-yl)-6-(trifluoromethyl)pyridazine-4-carboxylic acid (200 mg, 0.63 mmol) and tert-butyl ((3-aminophenyl)(methyl)(oxo)-λ⁶-sulfaneylidene)carbamate (205 mg, 0.76 mmol) in pyridine (10 mL) was added POCl₃ (10 mg, 0.063 mmol). The mixture was stirred at room temperate for 1 hours. Then the mixture was diluted with water (20 mL) and extracted with EtOAc (20 mL×3). The combined organic phases were washed with brine, dried over sodium sulfate, concentrated under vacuum. The residue was purified by flash column chromatography on silica gel (PE/EtOAc=5/1) to afford tert-butyl (methyl(3-(5-methyl-3-(6-azaspiro[2.5]octan-6-yl)-6-(trifluoromethyl)pyridazine-4-carboxamido)phenyl)(oxo)-λ⁶-sulfaneylidene)carbamate (70 mg, 21.95%) as a yellow solid. LCMS (ESI) calcd. for C₂₆H₃₃F₃N₅O₄S [M+H]⁺ m/z 569.22, found 568.25.

Step 6: 5-methyl-N-(3-(S-methylsulfonimidoyl)phenyl)-3-(6-azaspiro[2.5]octan-6-yl)-6-(trifluoromethyl)pyridazine-4-carboxamide: To a solution of tert-butyl (methyl(3-(5-methyl-3-(6-azaspiro[2.5]octan-6-yl)-6-(trifluoromethyl)pyridazine-4-carboxamido)phenyl)(oxo)-λ⁶-sulfaneylidene)carbamate (70 mg, 0.12 mmol) in DCM (1 mL) was added TFA (0.3 mL) at 0° C. The mixture was stirred at room temperature for 1 hour. After the reaction was completed, the mixture was diluted with water (10 mL) and extracted with DCM (10 mL×3). The combined organic phases were washed with brine, dried over sodium sulfate, concentrated under vacuum. The residue was purified by prep-HPLC (Gemini 5 um C₁₈ column, 150*21.2 mm, eluting with 30% to 85% MeCN/H₂O containing 0.1% formic acid) to provide 5-methyl-N-(3-(S-methylsulfonimidoyl)phenyl)-3-(6-azaspiro[2.5]octan-6-yl)-6-(trifluoromethyl)pyridazine-4-carboxamide (29 mg, 33.5%) as a white solid. ¹H NMR (400 MHz, DMSO-d₆) δ 11.14 (s, 1H), 8.30 (s, 1H), 7.86 (d, J=9.0 Hz, 1H), 7.72 (d, J=7.4 Hz, 1H), 7.62 (t, J=8.0 Hz, 1H), 4.26 (s, 1H), 3.76-3.62 (m, 4H), 3.07 (s, 3H), 2.33 (s, 3H), 1.43-1.32 (m, 4H), 0.31 (s, 4H). LCMS (ESI) calcd. for C₂₁H₂₅F₃N₅O₂S [M+H]⁺ m/z 468.17, found 468.05.

### Example 95

### 5-chloro-2-(4,4-difluoroazepan-1-yl)-4-methyl-N-(3-(S-methylsulfonimidoyl)phenyl)nicotinamide

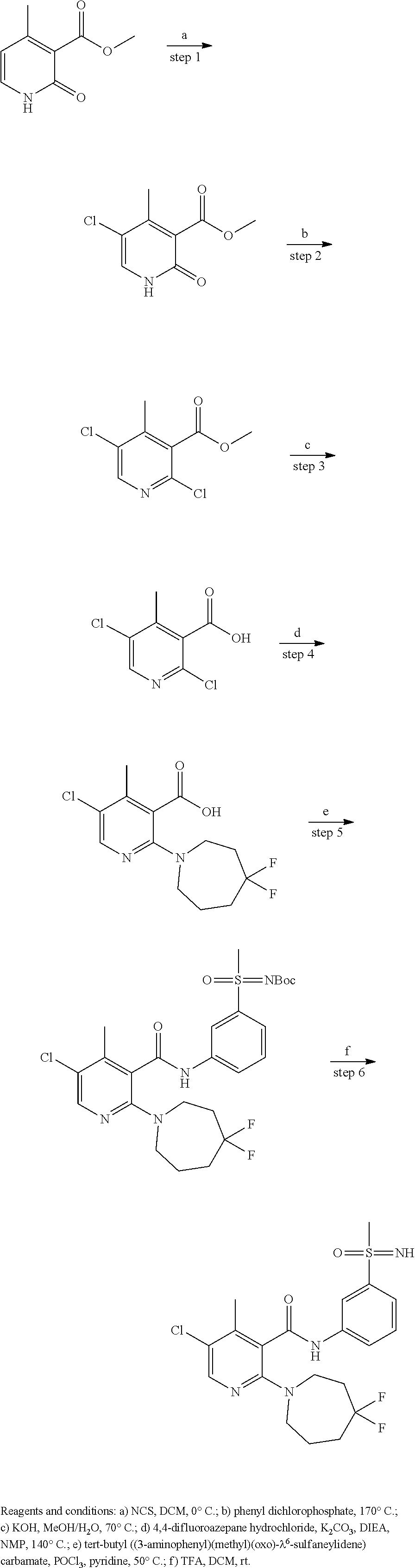

Step 1: methyl 5-chloro-2-hydroxy-4-methylnicotinate: To a solution of methyl 4-methyl-2-oxo-1,2-dihydropyridine-3-carboxylate (5.0 g, 30 mmol) in DCM (50 mL) was added NCS (4.0 g, 30 mmol) at 0° C. The mixture was stirred at the same temperature for 60 minutes. Then the mixture was quenched with water (50 mL) and extracted with DCM (2×50 mL). The combined organic phases were washed with brine, dried over sodium sulfate, concentrated under vacuum. The residue was purified by triturate in PE/EtOAc (3/1) to provide methyl 5-chloro-2-hydroxy-4-methylnicotinate (4 g, 66%) as a light brown solid. LCMS (ESI) calcd. for C₈H₉ClNO₃ [M+H]⁺ m/z 202.03, found 202.10.

Step 2: methyl 2,5-dichloro-4-methylnicotinate: A solution of methyl 5-chloro-2-hydroxy-4-methylnicotinate (2.0 g, 10 mmol) in phenyl dichlorophosphate (10 mL) was heated to 170° C. for 2 h. The resulting solution was cooled to room temperature, quenched with water (50 mL) and extracted with EtOAc (30 mL×3). The combined organic phases were washed with brine, dried over sodium sulfate, concentrated under vacuum. The residue was purified by flash column chromatography on silica gel (PE/EtOAc=10/1) to provide methyl 2,5-dichloro-4-methylnicotinate (1 g, 45%) as a light-yellow oil. LCMS (ESI) calcd. for C₈H₈Cl₂NO₂ [M+H]⁺ m/z 219.99, found 219.80.

Step 3: 2,5-dichloro-4-methylnicotinic acid: To a solution of methyl 2,5-dichloro-4-methylnicotinate (500 mg, 2.3 mmol) in MeOH/H₂O (v/v, 10 mL) was added KOH (129 mg, 23 mmol) at room temperature. The mixture was heated at 70° C. for 5 hours. After the reaction was completed, the mixture was concentrated to remove most MeOH. The aqueous phase was adjusted to pH=3-4 with 1N HCl then extracted with EtOAc (20 mL×3). The combined organic phases were washed with brine, dried with Na₂SO₄, and concentrated under reduced pressure to afford 2,5-dichloro-4-methylnicotinic acid (400 mg, 85.6%) as a white solid. LCMS (ESI) calcd. for C₇H₆C₁₂NO₂ [M+H]⁺ m/z 205.98, found 206.05.

Step 4: 5-chloro-2-(4,4-difluoroazepan-1-yl)-4-methylnicotinic acid: A solution of 2,5-dichloro-4-methylnicotinic acid (400 mg, 1.94 mmol), 4,4-difluoroazepane hydrochloride (498 mg, 2.91 mmol), K₂CO₃ (1.61 g, 11.64 mmol) and DIEA (1.0 g, 7.76 mmol) in NMP (10 mL) was heated at 140° C. for 6 hours. LCMS showed the reaction was completed. The mixture concentrated under vacuum and directly purified by flash column chromatography on silica gel (PE/EtOAc=2/1 to 1/2) to provide 5-chloro-2-(4,4-difluoroazepan-1-yl)-4-methylnicotinic acid (300 mg, 40%) as a yellow solid. LCMS (ESI) calcd. for C₁₃H₁₆ClF₂N₂O₂ [M+H]⁺ m/z 305.09, found 305.05.

Step 5: tert-butyl ((3-(5-chloro-2-(4,4-difluoroazepan-1-yl)-4-methylnicotinamido)phenyl)(methyl)(oxo)-λ⁶-sulfaneylidene)carbamate: A mixture of 5-chloro-2-(4,4-difluoroazepan-1-yl)-4-methylnicotinic acid (300 mg, 0.98 mmol) and tert-butyl ((3-aminophenyl) (methyl)(oxo)-λ⁶-sulfaneylidene) carbamate (264 mg, 0.98 mmol) in pyridine (6 mL) was added POCl₃ (200 L) dropwise at 50° C. The reaction solution was stirred at 50° C. for 1 hour. After the reaction was completed, the resulting solution was diluted with water (20 mL) and extracted with DCM (20 mL×3). The combined organic phases were washed with brine, dried over sodium sulfate, concentrated under vacuum. The residue was purified by flash column chromatography on silica gel (PE/EtOAc=2/1) to give tert-butyl ((3-(5-chloro-2-(4,4-difluoroazepan-1-yl)-4-methylnicotinamido)phenyl)(methyl)(oxo)-λ⁶-sulfaneylidene)carbamate (50 mg, 22.8%) as a yellow solid. LCMS (ESI) calcd. for C₂₅H₃₂ClF₂N₄O₄S [M+H]⁺ m/z 557.18, found 557.15.

Step 6: 5-chloro-2-(4,4-difluoroazepan-1-yl)-4-methyl-N-(3-(S-methylsulfonimidoyl)phenyl)nicotinamide: A solution of tert-butyl ((3-(5-chloro-2-(4,4-difluoroazepan-1-yl)-4-methylnicotinamido)phenyl)(methyl)(oxo)-λ⁶-sulfaneylidene)carbamate (50 mg, 0.089 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 30% to 85% MeCN/H₂O containing 0.1% formic acid) to provide 5-chloro-2-(4,4-difluoroazepan-1-yl)-4-methyl-N-(3-(S-methylsulfonimidoyl)phenyl)nicotinamide (14.9 mg, 36.3%) as a white solid. ¹H NMR (400 MHz, DMSO-d₆) δ 10.88 (s, 1H), 8.32 (s, 1H), 8.22 (s, 1H), 7.89 (d, J=7.8 Hz, 1H), 7.68 (d, J=8.2 Hz, 1H), 7.60 (t, J=7.8 Hz, 1H), 4.23 (s, 1H), 3.60 (s, 2H), 3.53 (t, J=6.1 Hz, 2H), 3.07 (s, 3H), 2.28 (s, 5H), 1.98 (s, 2H), 1.80 (d, J=5.5 Hz, 2H). LCMS (ESI) calcd. for C₂₀H₂₄ClF₂N₄O₂S [M+H]⁺ m/z 457.13, found 457.15.

### Example 96

### 2-(4,4-difluoroazepan-1-yl)-4-methyl-N-(3-(S-methylsulfonimidoyl)phenyl)-5-(p-tolyl)nicotinamide

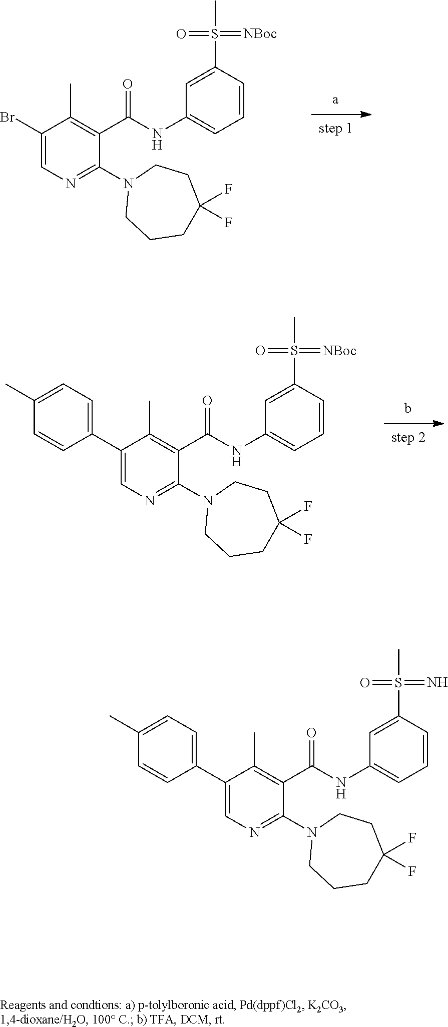

Step 1: tert-butyl ((3-(2-(4,4-difluoroazepan-1-yl)-4-methyl-5-(p-tolyl)nicotinamido)phenyl)(methyl)(oxo)-λ⁶-sulfaneylidene)carbamate: A mixture of tert-butyl ((3-(5-bromo-2-(4,4-difluoroazepan-1-yl)-4-methylnicotinamido)phenyl)(methyl)(oxo)-λ⁶-sulfaneylidene)carbamate (100 mg, 0.166 mmol), p-tolylboronic acid (68.0 mg, 0.50 mmol), K₂CO₃ (69.0 mg, 0.50 mmol) and [1,1′-Bis(diphenylphosphino)ferrocene]dichloropalladium (II) (12 mg, 0.016 mmol) in 1,4-dioxane/H₂O (4/1, 4 mL) was heated at 100° C. for 16 h under an atmosphere of N₂. After the reaction was completed, the mixture was cooled to room temperature. The resulting solution was diluted with water (30 mL) and extracted with EtOAc (30 mL×3). The combined organic phases were washed with brine, dried over Na₂SO₄, concentrated under vacuum. The residue was purified by flash column chromatography on silica gel (PE/EtAOc=1/1) to give tert-butyl ((3-(2-(4,4-difluoroazepan-1-yl)-4-methyl-5-(p-tolyl)nicotinamido)phenyl)(methyl)(oxo)-λ⁶-sulfaneylidene)carbamate (90 mg, 88.5%) as a white oil. LCMS (ESI) calcd. for C₃₂H₃₉F₂N₄O₄S [M+H]⁺ m/z 613.27, found 613.35.

Step 2: 2-(4,4-difluoroazepan-1-yl)-4-methyl-N-(3-(S-methylsulfonimidoyl)phenyl)-5-(p-tolyl)nicotinamide: A solution of give tert-butyl ((3-(2-(4,4-difluoroazepan-1-yl)-4-methyl-5-(p-tolyl)nicotinamido)phenyl)(methyl)(oxo)-λ⁶-sulfaneylidene)carbamate (90 mg, 0.147 mmol) in DCM (3 mL) was added TFA (1 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.05% NH₃) to provide 2-(4,4-difluoroazepan-1-yl)-4-methyl-N-(3-(S-methylsulfonimidoyl)phenyl)-5-(p-tolyl)nicotinamide (50.59 mg, 67.2%) as a white solid. ¹H NMR (400 MHz, DMSO-d₆) δ 10.86 (s, 1H), 8.38 (s, 1H), 8.05 (s, 1H), 7.90 (d, J=8.1 Hz, 1H), 7.66 (d, J=7.9 Hz, 1H), 7.58 (t, J=7.9 Hz, 1H), 7.27 (d, J=7.9 Hz, 2H), 7.22 (d, J=8.0 Hz, 2H), 4.22 (s, 1H), 3.64 (d, J=2.5 Hz, 2H), 3.57 (t, J=6.1 Hz, 2H), 3.05 (s, 3H), 2.35 (s, 3H), 2.28 (s, 2H), 2.15 (s, 3H), 2.00 (s, 2H), 1.82 (d, J=5.5 Hz, 2H). LCMS (ESI) calcd. for C₂₇H₃₁F₂N₄O₂S [M+H]⁺ m/z 513.21, found 513.20.

### Example 97

### 5-cyano-2-(4,4-difluoroazepan-1-yl)-4-methyl-N-(3-(S-methylsulfonimidoyl)phenyl)nicotinamide

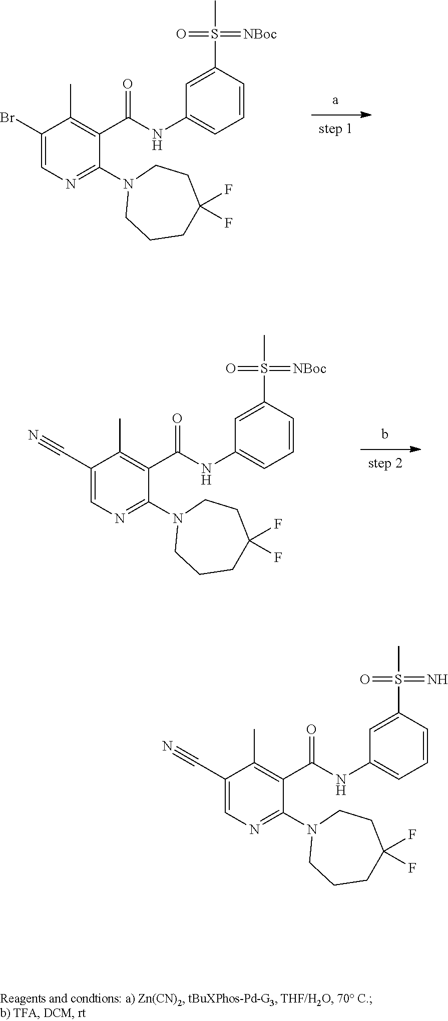

Step 1: tert-butyl ((3-(5-cyano-2-(4,4-difluoroazepan-1-yl)-4-methylnicotinamido)phenyl)(methyl)(oxo)-λ⁶-sulfaneylidene)carbamate: A mixture of tert-butyl ((3-(5-bromo-2-(4,4-difluoroazepan-1-yl)-4-methylnicotinamido)phenyl)(methyl)(oxo)-λ⁶-sulfaneylidene)carbamate (100 mg, 0.167 mmol), Zn(CN)₂ (39 mg, 0.334 mmol), tBuXPhos-Pd-G₃ (13 mg, 0.017 mmol) in THF/H₂O (4/1, 10 mL) was heated at 70° C. for 16 hours under an atmosphere of N₂. After the reaction was completed, the mixture was concentrated under vacuum. The residue was purified by flash column chromatography on silica gel (PE/EtAOc=1/1) to give tert-butyl ((3-(5-cyano-2-(4,4-difluoroazepan-1-yl)-4-methylnicotinamido)phenyl)(methyl)(oxo)-λ⁶-sulfaneylidene)carbamate (70 mg, 76.92%) as a yellow oil. LCMS (ESI) calcd. for C₂₆H₃₂F₂N₅O₄S [M+H]⁺ m/z 548.21, found 548.20.

Step 2: 5-cyano-2-(4,4-difluoroazepan-1-yl)-4-methyl-N-(3-(S-methylsulfonimidoyl)phenyl)nicotinamide: A solution of tert-butyl ((3-(5-cyano-2-(4,4-difluoroazepan-1-yl)-4-methylnicotinamido)phenyl)(methyl)(oxo)-λ⁶-sulfaneylidene)carbamate (70 mg, 0.128 mmol) in DCM (5 mL) was added TFA (0.5 mL) at room temperature. The solution was stirred at room temperature for 2 hours then concentrated under vacuum. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 55% to 90% MeCN/H₂O containing 0.1% formic acid) to obtain 5-cyano-2-(4,4-difluoroazepan-1-yl)-4-methyl-N-(3-(S-methylsulfonimidoyl)phenyl)nicotinamide (20.5 mg, 32.79%) as a white solid. ¹H NMR (400 MHz, DMSO-d₆) δ 10.91 (s, 1H), 8.56 (s, 1H), 8.29 (t, J=1.8 Hz, 1H), 7.93-7.84 (m, 1H), 7.74-7.65 (m, 1H), 7.60 (t, J=7.9 Hz, 1H), 4.24 (s, 1H), 3.62-3.32 (m, 4H), 3.06 (d, J=0.9 Hz, 3H), 2.40-2.21 (m, 5H), 2.08-1.93 (m, 2H), 1.89-1.78 (m, 2H). LCMS (ESI) calcd. for C₂₁H₂₄F₂N₅O₂S [M+H]⁺ m/z 448.16, found 448.05.

### Example 98

### 2-(4,4-difluoroazepan-1-yl)-4-methyl-N-(3-(S-methylsulfonimidoyl)phenyl)-5-(thiazol-2-yl)nicotinamide

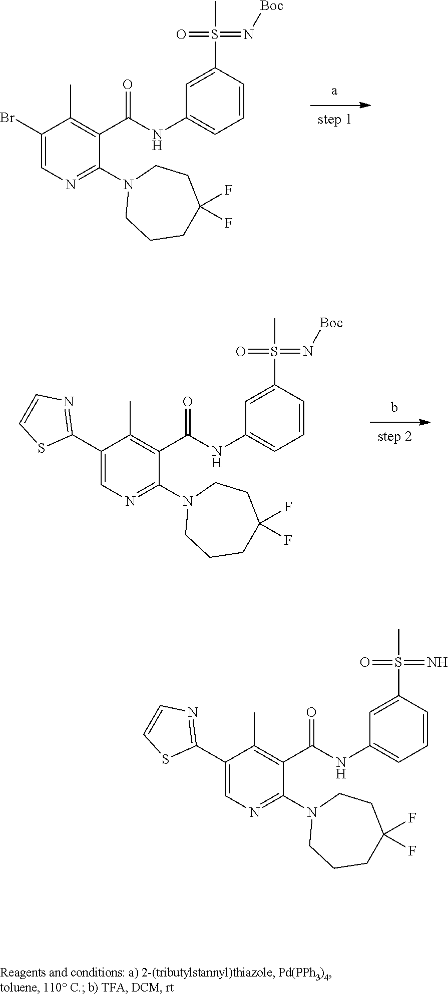 [SMILES: Cc1c(Br)cnc(N2CCCC(F)(F)CC2)c1C(=O)Nc1cccc(S(C)(=O)=NC(=O)OC(C)(C)C)c1 ; Cc1c(-c2nccs2)cnc(N2CCCC(F)(F)CC2)c1C(=O)Nc1cccc(S(C)(=O)=NC(=O)OC(C)(C)C)c1 ; Cc1c(-c2nccs2)cnc(N2CCCC(F)(F)CC2)c1C(=O)Nc1cccc(S(C)(=N)=O)c1]

Step 1: tert-butyl ((3-(2-(4,4-difluoroazepan-1-yl)-4-methyl-5-(thiazol-2-yl)nicotinamido)phenyl)(methyl)(oxo)-λ⁶-sulfaneylidene)carbamate: A mixture tert-butyl ((3-(5-bromo-2-(4,4-difluoroazepan-1-yl)-4-methylnicotinamido)phenyl)(methyl)(oxo)-λ⁶-sulfaneylidene)carbamate (100 mg, 0.16 mmol), 2-(tributylstannyl)thiazole (125 mg, 0.32 mmol) and Pd(PPh₃)₄ (20 mg, 0.02 mmol) in toluene (5 mL) was heated at 110° C. for 16 h under an atmosphere of N₂. After the reaction was completed, the mixture was cooled to room temperature. The resulting solution was diluted with water (20 mL) and extracted with EtOAc (20 mL×3). The combined organic phases were washed with brine, dried over Na₂SO₄, concentrated under vacuum. The residue was purified by flash column chromatography on silica gel (PE/EtOAc=3/1) to give tert-butyl ((3-(2-(4,4-difluoroazepan-1-yl)-4-methyl-5-(thiazol-2-yl)nicotinamido)phenyl)(methyl)(oxo)-λ⁶-sulfaneylidene)carbamate (75 mg, 77.3%). LCMS (ESI) calcd. for C₂₈H₃₄F₂N₅O₄S₂ [M+H]⁺ m/z 606.20, found 606.15.

Step 2: 2-(4,4-difluoroazepan-1-yl)-4-methyl-N-(3-(S-methylsulfonimidoyl)phenyl)-5-(thiazol-2-yl)nicotinamide: A solution of tert-butyl ((3-(2-(4,4-difluoroazepan-1-yl)-4-methyl-5-(thiazol-2-yl)nicotinamido)phenyl)(methyl)(oxo)-λ⁶-sulfaneylidene)carbamate (75 mg, 0.12 mmol) in DCM (3 mL) was added TFA (0.3 mL) at room temperature. The reaction mixture was stirred at room temperature for 2 hours. After the reaction was completed, the mixture was concentrated, and the residue was adjusted to pH=8-9 with saturated aqueous NaHCO₃. The aqueous solution was extracted with DCM (30 mL×3). The combined organic phases were washed with brine, dried over sodium sulfate, concentrated under vacuum. The residue was purified by prep-HPLC (Gemini 5 um C₁₈ column, 150*21.2 mm, eluting with 35% to 95% MeCN/H₂O containing 0.1% NH₃) to provide 2-(4,4-difluoroazepan-1-yl)-4-methyl-N-(3-(S-methylsulfonimidoyl)phenyl)-5-(thiazol-2-yl)nicotinamide (17.5 mg, 29.6%) as a white solid. ¹H NMR (400 MHz, DMSO-d₆) δ 10.95 (s, 1H), 8.48 (s, 1H), 8.36 (s, 1H), 7.97 (d, J=3.3 Hz, 1H), 7.91 (d, J=8.0 Hz, 1H), 7.82 (d, J=3.3 Hz, 1H), 7.68 (d, J=7.9 Hz, 1H), 7.60 (t, J=7.9 Hz, 1H), 4.23 (s, 1H), 3.75-3.60 (m, 4H), 3.07 (s, 3H), 2.43 (s, 3H), 2.31 (t, J=16.0 Hz, 2H), 2.09-1.96 (m, 2H), 1.87-1.80 (m, 2H). LCMS (ESI) calcd. for C₂₃H₂₆F₂N₅O₂S₂ [M+H]⁺ m/z 506.15, found 506.05.

### Example 99

### 6 C(4,4-difluoroazepan-1-yl)-4methyl-N-(3-(S-methylsulfonimidoyl)phenyl)-[2,3bipyridine]-5carboxamide

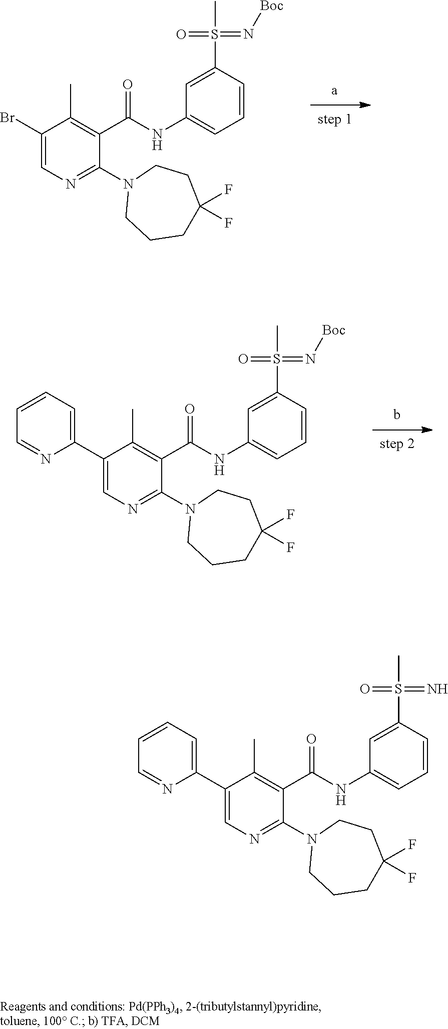 [SMILES: Cc1c(Br)cnc(N2CCCC(F)(F)CC2)c1C(=O)Nc1cccc(S(C)(=O)=NC(=O)OC(C)(C)C)c1 ; Cc1c(-c2ccccn2)cnc(N2CCCC(F)(F)CC2)c1C(=O)Nc1cccc(S(C)(=O)=NC(=O)OC(C)(C)C)c1 ; Cc1c(-c2ccccn2)cnc(N2CCCC(F)(F)CC2)c1C(=O)Nc1cccc(S(C)(=N)=O)c1]

Step 1: tert-butyl [symbol] ((3-(6 [symbol] (4,4-difluoroazepan-1-yl)-4 [symbol] methyl-[2,3 [symbol] bipyridine]-5 [symbol] carboxamido)phenyl)(methyl)(oxo)-λ⁶-sulfaneylidene)carbamate: A mixture tert-butyl ((3-(5-bromo-2-(4,4-difluoroazepan-1-yl)-4-methylnicotinamido)phenyl)(methyl)(oxo)-λ⁶-sulfaneylidene)carbamate (100 mg, 0.16 mmol), 2-(tributylstannyl)pyridine (118 mg, 0.32 mmol) and Pd(PPh₃)₄ (20 mg, 0.02 mmol) in toluene (5 mL) was heated at 100° C. for 16 h under an atmosphere of N₂. After the reaction was completed, the mixture was cooled to room temperature. The resulting solution was diluted with water (20 mL) and extracted with EtOAc (20 mL×3). The combined organic phases were washed with brine, dried over Na₂SO₄, concentrated under vacuum. The residue was purified by flash column chromatography on silica gel (PE/EtOAc=3/1) to give tert-butyl ((3-(6 [symbol] (4,4 [symbol] difluoroazepan-1-yl)-4 [symbol] methyl-[2,3 [symbol] bipyridine]-5 [symbol] carboxamido)phenyl)(methyl)(oxo)-λ⁶-sulfaneylidene)carbamate (52 mg, 54.2% y) as a white oil. LCMS (ESI) calcd. for C₃₀H₃₆F₂N₅O₄S [M+H]⁺ m/z 600.25, found 600.25.

Step 2: 6 [symbol] (4,4-difluoroazepan-1-yl)-4 [symbol] methyl-N-(3-(S-methylsulfonimidoyl)phenyl)-[2,3 [symbol] bipyridine]-5 [symbol] carboxamide: A solution of tert-butyl ((3-(61-(4,4-difluoroazepan-1-yl)-4 [symbol] methyl-[2,3 [symbol] bipyridine]-5 [symbol] carboxamido)phenyl)(methyl)(oxo)-λ⁶-sulfaneylidene)carbamate (52 mg, 0.09 mmol) in DCM (3 mL) was added TFA (0.3 mL) at room temperature. The reaction mixture was stirred at room temperature for 2 hours. After the reaction was completed, the mixture was concentrated, and the residue was adjusted to pH=8-9 with saturated aqueous NaHCO₃. The aqueous solution was extracted with DCM (30 mL×3). The combined organic phases were washed with brine, dried over sodium sulfate, concentrated under vacuum. The residue was purified by prep-HPLC (Gemini 5 um C₁₈ column, 150*21.2 mm, eluting with 30% to 90% MeCN/H₂O containing 0.1% NH₃) to provide 6 [symbol] (4,4-difluoroazepan-1-yl)-4 [symbol] methyl-N-(3-(S-methylsulfonimidoyl)phenyl)-[2,3 [symbol] bipyridine]-5 [symbol] carboxamide (16.58 mg, 36.8%) as a white solid. ¹H NMR (400 MHz, DMSO-d₆) δ 10.91 (s, 1H), 8.67 (d, J=4.7 Hz, 1H), 8.38 (s, 1H), 8.25 (s, 1H), 7.95-7.83 (m, 2H), 7.67 (d, J=7.9 Hz, 1H), 7.63-7.49 (m, 2H), 7.36 (dd, J=7.4, 4.9 Hz, 1H), 4.22 (s, 1H), 3.72-3.57 (m, 4H), 3.06 (s, 3H), 2.38-2.22 (m, 5H), 2.10-1.95 (m, 2H), 1.83 (dt, J=11.2, 5.8 Hz, 2H). LCMS (ESI) calcd. for C₂₅H₂₈F₂N₅O₂S [M+H]⁺ m/z 500.19, found 500.10.

### Example 100

### 5-methyl-N-(3-(S-methylsulfonimidoyl)phenyl)-3-(2-oxa-6-azaspiro[3.3]heptan-6-yl)-6-(trifluoromethyl)pyridazine-4-carboxamide

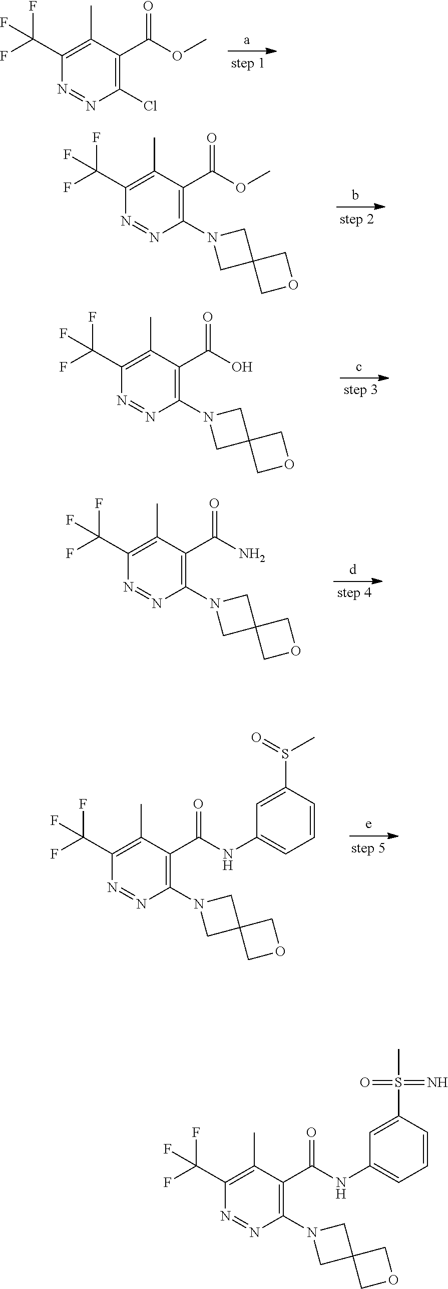

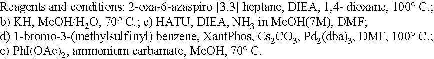

Step 1: methyl 5-methyl-3-(2-oxa-6-azaspiro[3.3]heptan-6-yl)-6-(trifluoromethyl)pyridazine-4-carboxylate: To a solution of methyl 3-chloro-5-methyl-6-(trifluoromethyl) pyridazine-4-carboxylate (400 mg, 1.57 mmol) in 1,4-dioxane (5 mL) was added 2-oxa-6-azaspiro [3.3]heptane (186 mg, 1.88 mmol) and DIPEA (304 mg, 2.35 mmol). The mixture was heated to 100° C. and stirred for 2 hours. Then the mixture was concentrated under vacuum. The residue was purified by flash column chromatography on silica gel (PE/EtOAc=1/1) to provide methyl 5-methyl-3-(2-oxa-6-azaspiro[3.3]heptan-6-yl)-6-(trifluoromethyl)pyridazine-4-carboxylate (400 mg, 88%) as a light brown solid. LCMS (ESI) calcd. for C₁₃H₁₅F₃N₃O₃ [M+H]⁺ m/z 318.11, found 318.05.

Step 2: 5-methyl-3-(2-oxa-6-azaspiro[3.3]heptan-6-yl)-6-(trifluoromethyl)pyridazine-4-carboxylic acid: To a solution of methyl 5-methyl-3-(2-oxa-6-azaspiro[3.3]heptan-6-yl)-6-(trifluoromethyl)pyridazine-4-carboxylate (400 mg, 1.26 mmol) in MeOH/H₂O (1/1, 10 mL) was added KOH (705 mg, 12.6 mmol) at room temperature. The mixture was heated at 70° C. for 5 hours. After the reaction was completed, the mixture was concentrated to remove most MeOH. The aqueous phase was adjusted to pH=3-4 with 1N HCl then extracted with EtOAc (20 mL×3). The combined organic phases were washed with brine, dried with Na₂SO₄, and concentrated under reduced pressure to afford 5-methyl-3-(2-oxa-6-azaspiro[3.3]heptan-6-yl)-6-(trifluoromethyl)pyridazine-4-carboxylic acid (300 mg, 78.5%) as a white solid. LCMS (ESI) calcd. for C₁₂H₁₃F₃N₃O₃ [M+H]⁺ m/z 304.09, found 304.00.

Step 3: 5-methyl-3-(2-oxa-6-azaspiro[3.3]heptan-6-yl)-6-(trifluoromethyl)pyridazine-4-carboxamide: To a solution of 5-methyl-3-(2-oxa-6-azaspiro[3.3]heptan-6-yl)-6-(trifluoromethyl)pyridazine-4-carboxylic acid (200 mg, 0.66 mmol) and HATU (300 mg, 0.79 mmol) in DMF (5 mL) was added DIPEA (128 mg, 0.99 mmol). The mixture was heated to 40° C. and stirred for 2 hours. Then NH₃-MeOH (3.3 mL, 7M) was added, the mixture was stirred for 5 hours at 40° C. After the reaction was completed, the mixture was concentrated under vacuum. The residue was purified by flash column chromatography on silica gel (PE/EtOAc=1/1) to provide 5-methyl-3-(2-oxa-6-azaspiro[3.3]heptan-6-yl)-6-(trifluoromethyl)pyridazine-4-carboxamide (120 mg, 60.3%) as a yellow solid. LCMS (ESI) calcd. for C₁₂H₁₄F₃N₄O₂ [M+H]+m/z 303.11, found 303.10.

Step 4: 5-methyl-N-(3-(methylsulfinyl)phenyl)-3-(2-oxa-6-azaspiro[3.3]heptan-6-yl)-6-(trifluoromethyl)pyridazine-4-carboxamide: A solution of 5-methyl-3-(2-oxa-6-azaspiro[3.3]heptan-6-yl)-6-(trifluoromethyl)pyridazine-4-carboxamide (120 mg, 0.40 mmol), 1-bromo-3-(methylsulfinyl) benzene (105 mg, 0.48 mmol), Cs₂CO₃ (261 mg, 0.80 mmol), XantPhos (46 mg, 0.08 mmol) and Pd₂(dba)₃ (37 mg, 0.04 mmol) in DMF (2 mL) was heated at 100° C. overnight under an atmosphere of N₂. LCMS showed the reaction was completed. The mixture was filtered through celite, and the filtrate was concentrated under vacuum. The residue was purified by flash column chromatography on silica gel (PE/EtOAc=1/1) to give 5-methyl-N-(3-(methylsulfinyl)phenyl)-3-(2-oxa-6-azaspiro[3.3]heptan-6-yl)-6-(trifluoromethyl)pyridazine-4-carboxamide (90 mg, 51.7%) as a yellow oil. LCMS (ESI) calcd. for C₁₉H₂₀F₃N₄O₃S [M+H]⁺ m/z 441.12, found 441.10.

Step 5: 5-methyl-N-(3-(S-methylsulfonimidoyl)phenyl)-3-(2-oxa-6-azaspiro[3.3]heptan-6-yl)-6-(trifluoromethyl)pyridazine-4-carboxamide: To a solution of 5-methyl-N-(3-(methylsulfinyl)phenyl)-3-(2-oxa-6-azaspiro[3.3]heptan-6-yl)-6-(trifluoromethyl)pyridazine-4-carboxamide (90 mg, 0.20 mmol) in MeOH (5 mL) was added PhI(OAc)₂ (161 mg, 0.5 mmol) and ammonium carbamate (47 mg, 0.6 mmol) at room temperature. The reaction mixture was heated at 70° C. for 3 hours. Then the mixture was cooled to room temperature, diluted with water (20 mL) and extracted with DCM (30 mL×3). The combined organic phases were washed with brine, dried over sodium sulfate, concentrated under vacuum. The residue was purified by prep-HPLC (Gemini 5 um C₁₈ column, 150*21.2 mm, eluting with 50% to 55% MeCN/H₂O containing 0.1% formic acid) to afford 5-methyl-N-(3-(S-methylsulfonimidoyl)phenyl)-3-(2-oxa-6-azaspiro[3.3]heptan-6-yl)-6-(trifluoromethyl)pyridazine-4-carboxamide (17.9 mg, 19.3%) as a white solid. ¹H NMR (400 MHz, DMSO-d₆) δ 11.18 (s, 1H), 8.39 (s, 1H), 7.97 (d, J=9.1 Hz, 1H), 7.82 (d, J=8.4 Hz, 1H), 7.72 (t, J=8.0 Hz, 1H), 4.69 (s, 4H), 4.37 (s, 4H), 3.38 (s, 3H), 2.28 (s, 3H). LCMS (ESI) calcd. for C₁₉H₂₁F₃N₅O₃S [M+H]⁺ m/z 456.13, found 456.10.

### Example 101

### 2-(4,4-difluoroazepan-1-yl)-6-methyl-N-(2-(S-methylsulfonimidoyl)pyridin-4-yl)-5-(trifluoromethyl)nicotinamide

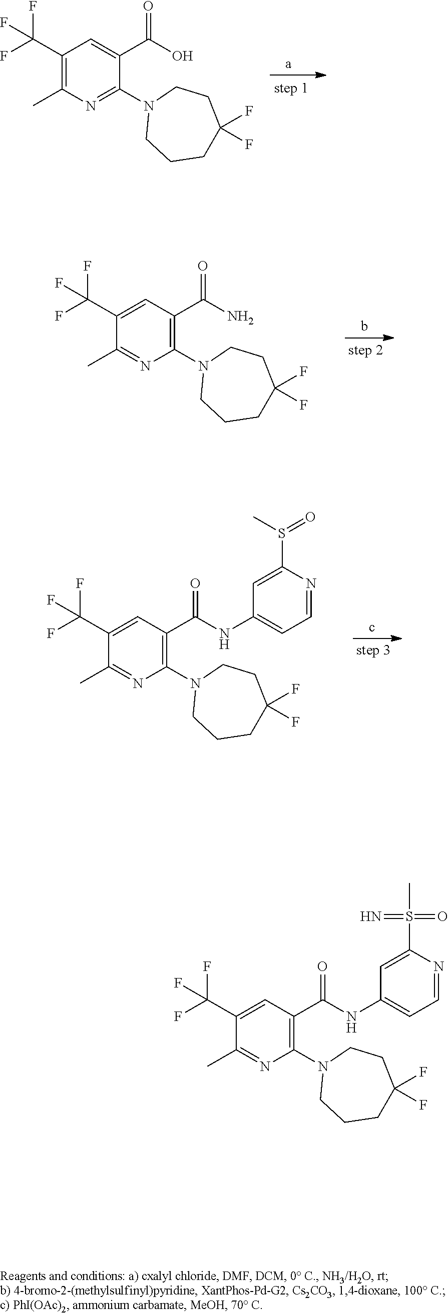 [SMILES: Cc1nc(N2CCCC(F)(F)CC2)c(C(=O)O)cc1C(F)(F)F ; Cc1nc(N2CCCC(F)(F)CC2)c(C(N)=O)cc1C(F)(F)F ; Cc1nc(N2CCCC(F)(F)CC2)c(C(=O)Nc2ccnc(S(C)=O)c2)cc1C(F)(F)F ; Cc1nc(N2CCCC(F)(F)CC2)c(C(=O)Nc2ccnc(S(C)(=N)=O)c2)cc1C(F)(F)F]

Step 1: 2-(4,4-difluoroazepan-1-yl)-6-methyl-5-(trifluoromethyl)nicotinamide: A solution of 2-(4,4-difluoroazepan-1-yl)-6-methyl-5-(trifluoromethyl)nicotinic acid (200 mg, 0.59 mmol) in DCM (5 mL) and DMF (1 drop) was added dropwise oxalyl chloride (86 μL) at 0° C. The mixture was stirred at 0° C. for 1 hour. Then the mixture was concentrated to remove the solvent. Then the residue was dissolved in THE (5 mL) and was added to vigorously stirred NH₃—H₂O (5 mL). The mixture was stirred at room temperature for 1 hour. The resulting mixture was diluted with water (10 mL) and extracted with EtOAc (20 mL×3). The combined organic phases were washed with water and brine, dried over sodium sulfate, and concentrated under vacuum to give crude 2-(4,4-difluoroazepan-1-yl)-6-methyl-5-(trifluoromethyl)nicotinamide (190 mg) which was used directly in next step. LCMS (ESI) calcd. for C₁₄H₁₇F₅N₃O [M+H]⁺ m/z 338.13, found 338.05.

Step 2: 2-(4,4-difluoroazepan-1-yl)-6-methyl-N-(2-(methylsulfinyl)pyridin-4-yl)-5-(trifluoromethyl)nicotinamide: A solution of 2-(4,4-difluoroazepan-1-yl)-6-methyl-5-(trifluoromethyl)nicotinamide (175 mg, 0.52 mmol) in 1,4-dioxane (8 mL) was added 4-bromo-2-(methylsulfinyl)pyridine (457.38 mg, 1.53 mmol), Cs₂CO₃ (440.5 mg, 1.35 mmol) and XantPhos-Pd-G2 (92.4 mg, 0.1 mmol) at room temperature. The reaction mixture was heated at 100° C. for 16 hours under nitrogen. The reaction was monitored by LCMS. After the reaction was completed, the mixture was diluted with water (20 mL) and extracted with DCM (20 mL×3). The combined organic phases were washed with brine, dried over sodium sulfate, concentrated under vacuum. The residue was purified by flash column chromatography on silica gel (PE/EtOAc=1/1) to give 2-(4,4-difluoroazepan-1-yl)-6-methyl-N-(2-(methylsulfinyl)pyridin-4-yl)-5-(trifluoromethyl)nicotinamide (120 mg, 48.5%) as a white solid. LCMS (ESI) calcd. for C₂₀H₂₂F₅N₄O₂S [M+H]⁺ m/z 477.14, found 477.10.

Step 3: 2-(4,4-difluoroazepan-1-yl)-6-methyl-N-(2-(S-methylsulfonimidoyl)pyridin-4-yl)-5-(trifluoromethyl)nicotinamide: To a solution of 2-(4,4-difluoroazepan-1-yl)-6-methyl-N-(2-(methylsulfinyl)pyridin-4-yl)-5-(trifluoromethyl)nicotinamide (120 mg, 0.252 mmol) in MeOH (5 mL) was added PhI(OAc)₂ (203 mg, 0.63 mmol) and ammonium carbamate (59 mg, 0.756 mmol) at room temperature. The reaction mixture was heated at 70° C. for 16 hours. After the reaction was completed, the mixture was cooled to room temperature, diluted with water (5 mL), and extracted with DCM (5 mL×3). The combined organic phases were washed with brine, dried over sodium sulfate and concentrated under vacuum. The residue was first purified by flash column chromatography on silica gel (PE/EtOAc=1/1), then further purified by prep-HPLC (Gemini 5 um C₁₈ column, 150*21.2 mm, eluting with 50% to 90% MeCN/H₂O containing 0.05% NH₃) to afford 2-(4,4-difluoroazepan-1-yl)-6-methyl-N-(2-(S-methylsulfonimidoyl)pyridin-4-yl)-5-(trifluoromethyl)nicotinamide (45.5 mg, 36.8%) as a white solid. ¹H NMR (400 MHz, DMSO-d₆) δ 11.16 (s, 1H), 8.60 (d, J=5.5 Hz, 1H), 8.37 (d, J=1.8 Hz, 1H), 8.01 (s, 1H), 7.87 (dd, J=5.5, 2.0 Hz, 1H), 4.35 (s, 1H), 3.73-3.67 (m, 2H), 3.41 (t, J=5.6 Hz, 2H), 3.14 (d, J=0.9 Hz, 3H), 2.52 (d, J=1.4 Hz, 3H), 2.38-2.27 (m, 2H), 1.95 (s, 2H), 1.88 (d, J=5.3 Hz, 2H). LCMS (ESI) calcd. for C₂₀H₂₃F₅N₅O₂S [M+H]⁺ m/z 492.15, found 492.20.

### Example 102

### 3-(4,4-difluoroazepan-1-yl)-5-methyl-N-(2-(S-methylsulfonimidoyl)pyridin-4-yl)-6-(trifluoromethyl)pyridazine-4-carboxamide

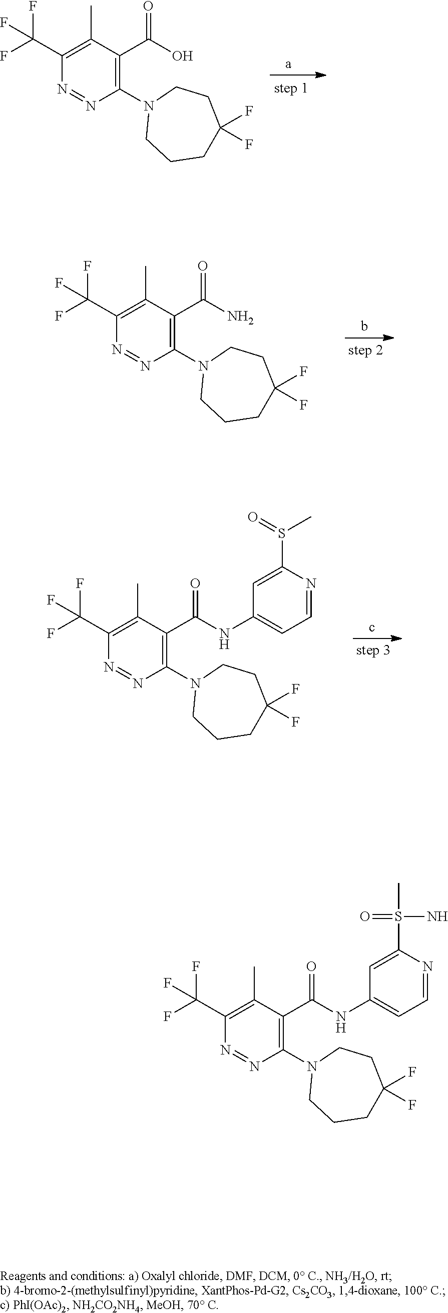

Step 1: 3-(4,4-difluoroazepan-1-yl)-5-methyl-6-(trifluoromethyl)pyridazine-4-carboxamide: A solution of 3-(4,4-difluoroazepan-1-yl)-5-methyl-6-(trifluoromethyl)pyridazine-4-carboxylic acid (250 mg, 0.7374 mmol) in DCM (5 mL) was added oxalyl chloride (0.3 mL) and DMF (1 drop) at room temperature. After addition, the reaction mixture was stirred at room temperature for 1 hour. After the reaction was completed, the reaction mixture was concentrated. The residue was diluted with THE and added dropwise to a stirred solution of ammonium hydroxide (5 mL). Then the mixture was stirred at room temperature for 1 hour. After the reaction was completed, the mixture was diluted with water (10 mL) and extracted with DCM (10 mL×3). The combined organic phases were washed with brine, dried over sodium sulfate, concentrated under vacuum. The residue was purified by flash column chromatography on silica gel (PE/EtOAc=1/1) to give 3-(4,4-difluoroazepan-1-yl)-5-methyl-6-(trifluoromethyl)pyridazine-4-carboxamide (200 mg, 80.10%) as a yellow solid. LCMS (ESI) calcd. for C₁₃H₁₆F₅N₄O [M+H]⁺ m/z 339.12, found 339.10.

Step 2: 3-(4,4-difluoroazepan-1-yl)-5-methyl-N-(2-(methylsulfinyl)pyridin-4-yl)-6-(trifluoromethyl)pyridazine-4-carboxamide: A solution of 3-(4,4-difluoroazepan-1-yl)-5-methyl-6-(trifluoromethyl)pyridazine-4-carboxamide (180 mg, 0.5325 mmol) in 1,4-dioxane (10 mL) was added 4-bromo-2-(methylsulfinyl)pyridine (349 mg, 1.597 mmol), cesium carbonate (451 mg, 1.3845 mmol) and Xantphos-Pd-G2 (94 mg, 0.1065 mmol) at room temperature. The reaction mixture was heated at 100° C. for 16 hours under nitrogen. After the reaction was completed, the mixture was diluted with water (20 mL) and extracted with DCM (20 mL×3). The combined organic phases were washed with brine, dried over sodium sulfate, concentrated under vacuum. The residue was purified by flash column chromatography on silica gel (PE/EtOAc=1/1) to give 3-(4,4-difluoroazepan-1-yl)-5-methyl-N-(2-(methylsulfinyl)pyridin-4-yl)-6-(trifluoromethyl)pyridazine-4-carboxamide (120 mg, 47.24%) as a yellow oil. LCMS (ESI) calcd. for C₁₉H₂₁F₅N₅O₂S [M+H]⁺ m/z 478.13, found 478.15.

Step 3: 3-(4,4-difluoroazepan-1-yl)-5-methyl-N-(2-(S-methylsulfonimidoyl)pyridin-4-yl)-6-(trifluoromethyl)pyridazine-4-carboxamide: To a solution of 3-(4,4-difluoroazepan-1-yl)-5-methyl-N-(2-(methylsulfinyl)pyridin-4-yl)-6-(trifluoromethyl)pyridazine-4-carboxamide (120 mg, 0.251 mmol) in MeOH (5 mL) was added PhI(OAc)₂ (202 mg, 0.627 mmol) and ammonium carbamate (58 mg, 0.753 mmol) at room temperature. The reaction mixture was heated at 70° C. for 5 hours. After the reaction was completed, the mixture was cooled to room temperature, diluted with water (5 mL), and extracted with DCM (5 mL×3). The combined organic phases were washed with brine, dried over sodium sulfate and concentrated under vacuum. The residue was first purified by flash column chromatography on silica gel (PE/EtOAc=1/1), then further purified by prep-HPLC (Gemini 5 um C₁₈ column, 150*21.2 mm, eluting with 35% to 95% MeCN/H₂O containing 0.1% formic acid) to afford 3-(4,4-difluoroazepan-1-yl)-5-methyl-N-(2-(S-methylsulfonimidoyl)pyridin-4-yl)-6-(trifluoromethyl)pyridazine-4-carboxamide (54.46 mg, 41.57%) as a white solid. ¹H NMR (400 MHz, DMSO-d₆) δ 11.50 (s, 1H), 8.66 (d, J=5.2 Hz, 1H), 8.35 (d, J=1.6 Hz, 1H), 7.80 (dd, J=5.2 Hz, 1.6 Hz, 1H), 4.42 (s, 1H), 3.83-3.81 (m, 2 H), 3.64-3.61 (m, 2H), 3.15 (s, 3H), 1.83-1.81 (m, 2H), 2.31 (s, 3H), 2.08-2.04 (m, 2H), 1.89-1.87 (m, 2H). LCMS (ESI) calcd. for C₁₉H₂₂F₅N₆O₂S [M+H]⁺ m/z 493.14, found 493.10.

### Example 103

### (S)-2-(4,4-difluoroazepan-1-yl)-4-methyl-N-(3-(S-methylsulfonimidoyl)phenyl)-5-phenylnicotinamide

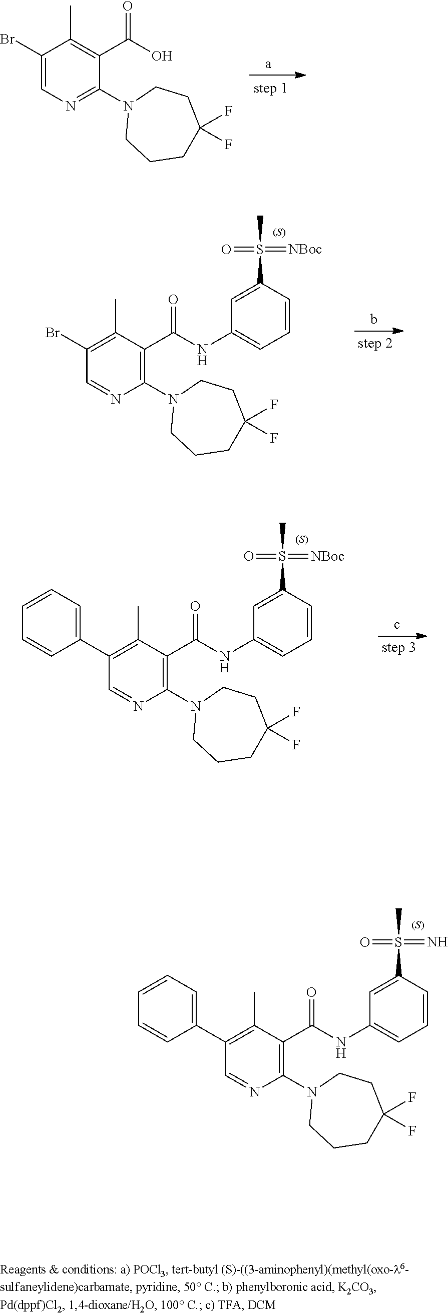

Step 1: tert-butyl (S)-((3-(5-bromo-2-(4,4-difluoroazepan-1-yl)-4-methylnicotinamido)phenyl)(methyl)(oxo)-λ⁶-sulfaneylidene)carbamate: A mixture of 5-bromo-2-(4,4-difluoroazepan-1-yl)-4-methylnicotinic acid (120 mg, 0.34 mmol) and tert-butyl (S)-((3-aminophenyl)(methyl)(oxo)-λ⁶-sulfaneylidene)carbamate (112 mg, 0.41 mmol) in pyridine (5 mL) was added POCl₃ (100 L) dropwise at 50° C. The reaction solution was stirred at 50° C. 1 hour. After the reaction was completed, the resulting solution was diluted with water (20 mL) and extracted with DCM (20 mL×3). The combined organic phases were washed with brine, dried over sodium sulfate, concentrated under vacuum. The residue was purified by flash column chromatography on silica gel (PE/EtOAc=2/1) to give tert-butyl (S)-((3-(5-bromo-2-(4,4-difluoroazepan-1-yl)-4-methylnicotinamido)phenyl)(methyl)(oxo)-λ⁶-sulfaneylidene)carbamate (60 mg, 29.3%) as a yellow solid. LCMS (ESI) calcd. for C₂₅H₃₂BrF₂N₄O₄S [M+H]⁺ m/z 603.13, found 603.10.

Step 2: tert-butyl (S)-((3-(2-(4,4-difluoroazepan-1-yl)-4-methyl-5-phenylnicotinamido)phenyl)(methyl)(oxo)-λ⁶-sulfaneylidene)carbamate: A mixture of tert-butyl (S)-((3-(5-bromo-2-(4,4-difluoroazepan-1-yl)-4-methylnicotinamido)phenyl)(methyl)(oxo)-λ⁶-sulfaneylidene)carbamate (60 mg, 0.10 mmol), phenylboronic acid (61 mg, 0.50 mmol), K₂CO₃ (42 mg, 0.30 mmol) and Pd(dppf)Cl₂ (7 mg, 0.01 mmol) in 1,4-dioxane/H₂O (4/1, 10 mL) was heated at 100° C. for 16 hours under an atmosphere of N₂. After the reaction was completed, the mixture was concentrated under vacuum. The residue was directly purified by flash column chromatography on silica gel (PE/EtAOc=3/1) to give tert-butyl (S)-((3-(2-(4,4-difluoroazepan-1-yl)-4-methyl-5-phenylnicotinamido)phenyl)(methyl)(oxo)-λ⁶-sulfaneylidene)carbamate (30 mg, 50.1%) as a yellow solid. LCMS (ESI) calcd. for C₃₁H₃₇F₂N₄O₄S [M+H]⁺ m/z 599.25, found 599.25.

Step 3: (S)-2-(4,4-difluoroazepan-1-yl)-4-methyl-N-(3-(S-methylsulfonimidoyl)phenyl)-5-phenylnicotinamide: A solution of tert-butyl (S)-((3-(2-(4,4-difluoroazepan-1-yl)-4-methyl-5-phenylnicotinamido)phenyl)(methyl)(oxo)-λ⁶-sulfaneylidene)carbamate (30 mg, 0.05 mmol) in DCM (3 mL) was added TFA (0.3 mL) at: room temperature. The reaction mixture was stirred at room temperature for 2 hours. After the reaction was completed, the mixture was concentrated, and the residue was adjusted to pH=8-9 with saturated aqueous NaHCO₃. The aqueous solution was extracted with DCM (30 mL×3). The combined organic phases were washed with brine, dried over sodium sulfate, concentrated under vacuum. The residue was purified by prep-HPLC (Gemini 5 um C₁₈ column, 150*21.2 mm, eluting with 40% to 95% MeCN/H₂O containing 0.1% NH₃) to provide (S)-2-(4,4-difluoroazepan-1-yl)-4-methyl-N-(3-(S-methylsulfonimidoyl)phenyl)-5-phenylnicotinamide (14.28 mg, 57.2%) as a white solid. ¹H NMR (400 MHz, DMSO-d₆) δ 10.88 (s, 1H), 8.38 (s, 1H), 8.08 (s, 1H), 7.90 (d, J=8.6 Hz, 1H), 7.67 (d, J=7.9 Hz, 1H), 7.58 (t, J=7.9 Hz, 1H), 7.47 (t, J=7.4 Hz, 2H), 7.42-7.30 (m, 3H), 4.22 (s, 1H), 3.69-3.54 (m, 4H), 3.06 (s, 3H), 2.29 (t, J=16.1 Hz, 2H), 2.16 (s, 3H), 2.05-1.96 (m, 2H), 1.86-1.76 (m, 2H). LCMS (ESI) calcd. for C₂₆H₂₉F₂N₄O₂S [M+H]⁺ m/z 499.20, found 499.10.

### Example 104

### 2-(4,4-difluoroazepan-1-yl)-5-(2,6-difluorophenyl)-4-methyl-N-(3-(S-methylsulfonimidoyl)phenyl)nicotinamide

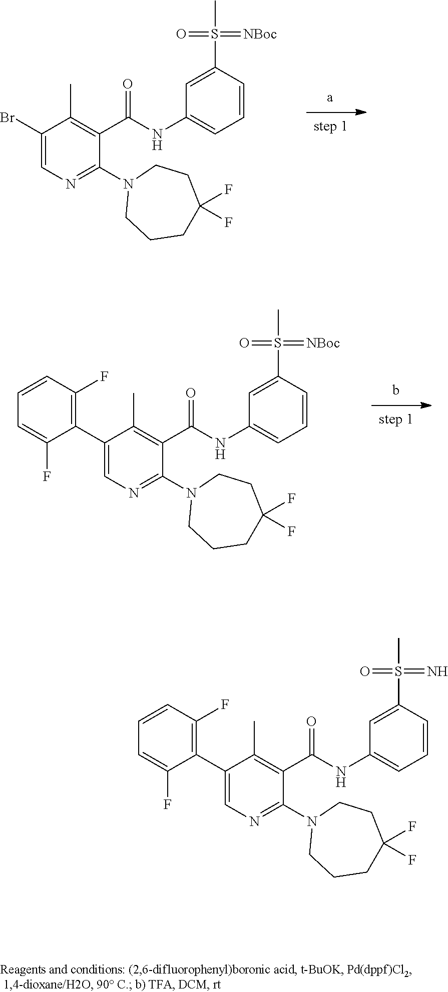

Step 1: tert-butyl ((3-(2-(4,4-difluoroazepan-1-yl)-5-(2,6-difluorophenyl)-4-methylnicotinamido)phenyl)(methyl)(oxo)-λ⁶-sulfaneylidene)carbamate: A mixture of tert-butyl ((3-(5-bromo-2-(4,4-difluoroazepan-1-yl)-4-methylnicotinamido)phenyl)(methyl)(oxo)-λ⁶-sulfaneylidene)carbamate (200 mg, 0.333 mmol), (2,6-difluorophenyl)boronic acid (105 mg, 0.667 mmol), tBuOK (75 mg, 0.667 mmol) and [1,1′-bis(diphenylphosphino)ferrocene]dichloropalladium (II) (24 mg, 0.033 mmol) in 1,4-dioxane/H₂O (4/1, 10 mL) was heated at 90° C. for 16 hours under an atmosphere of N₂. After the reaction was completed, the mixture was concentrated under vacuum. The residue was purified by flash column chromatography on silica gel (PE/EtAOc=1/1) to give tert-butyl ((3-(2-(4,4-difluoroazepan-1-yl)-5-(2,6-difluorophenyl)-4-methylnicotinamido)phenyl)(methyl)(oxo)-λ⁶-sulfaneylidene)carbamate (30 mg, 14.22%) as a yellow solid. LCMS (ESI) calcd. for C₃₁H₃₅F₄N₄O₄S [M+H]⁺ m/z 635.23, found 635.20.

Step 2: 2-(4,4-difluoroazepan-1-yl)-5-(2,6-difluorophenyl)-4-methyl-N-(3-(S-methylsulfonimidoyl)phenyl)nicotinamide: A solution of tert-butyl ((3-(2-(4,4-difluoroazepan-1-yl)-5-(2,6-difluorophenyl)-4-methylnicotinamido)phenyl)(methyl)(oxo)-λ⁶-sulfaneylidene)carbamate (30 mg, 0.047 mmol) in DCM (3 mL) was added TFA (0.3 mL) at room temperature. The solution was stirred at room temperature for 2 hours then concentrated under vacuum. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 55% to 90% MeCN/H₂O containing 0.1% formic acid) to obtain 2-(4,4-difluoroazepan-1-yl)-5-(2,6-difluorophenyl)-4-methyl-N-(3-(S-methylsulfonimidoyl)phenyl)nicotinamide (7 mg, 28.48%) as a white solid. ¹H NMR (400 MHz, DMSO-d₆) δ 10.94 (s, 1H), 8.38 (t, J=1.8 Hz, 1H), 8.08 (s, 1H), 7.91 (d, J=8.9 Hz, 1H), 7.69-7.49 (m, 3H), 7.25 (t, J=7.9 Hz, 2H), 4.24 (s, 1H), 3.70-3.57 (m, 4H), 3.06 (s, 3H), 2.36-2.24 (m, 2H), 2.08-1.98 (m, 5H), 1.86-1.78 (m, 2H). LCMS (ESI) calcd. for C₂₆H₂₇F₄N₄O₂S [M+H]⁺ m/z 535.18, found 535.15.

### Example 105

### 2-(4,4-difluoroazepan-1-yl)-4-methyl-N-(3-(S-methylsulfonimidoyl)phenyl)nicotinamide

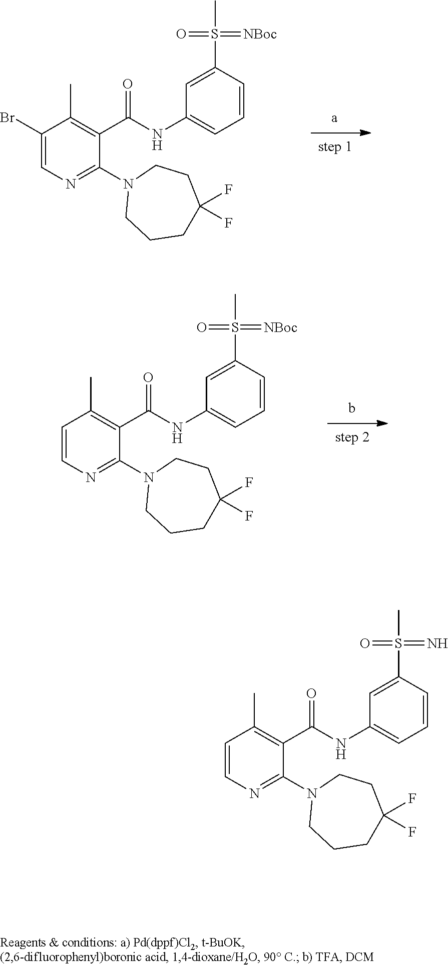

Step 1: tert-butyl ((3-(2-(4,4-difluoroazepan-1-yl)-4-methylnicotinamido)phenyl)(methyl)(oxo)-λ⁶-sulfaneylidene)carbamate: A mixture of tert-butyl ((3-(5-bromo-2-(4,4-difluoroazepan-1-yl)-4-methylnicotinamido)phenyl)(methyl)(oxo)-λ⁶-sulfaneylidene)carbamate (200 mg, 0.333 mmol), (2,6-difluorophenyl)boronic acid (105 mg, 0.667 mmol), t-BuOK (75 mg, 0.667 mmol) and [1,1′-Bis(diphenylphosphino)ferrocene]dichloropalladium (II) (24 mg, 0.033 mmol) in 1,4-dioxane/H₂O (4/1, 10 mL) was heated at 90° C. for 16 hours under an atmosphere of N₂. After the reaction was completed, the mixture was concentrated under vacuum. The residue was purified by flash column chromatography on silica gel (PE/EtAOc=1/2) to give tert-butyl ((3-(2-(4,4-difluoroazepan-1-yl)-4-methylnicotinamido)phenyl)(methyl)(oxo)-λ⁶-sulfaneylidene)carbamate (80 mg, 45.98%, yellow solid) as a byproduct of Suzuki coupling. LCMS (ESI) calcd. for C₂₅H₃₃F₂N₄O₄S [M+H]⁺ m/z 523.22, found 523.15.

Step 2: 2-(4,4-difluoroazepan-1-yl)-4-methyl-N-(3-(S-methylsulfonimidoyl)phenyl)nicotinamide: A solution of tert-butyl ((3-(2-(4,4-difluoroazepan-1-yl)-4-methylnicotinamido)phenyl)(methyl)(oxo)-λ⁶-sulfaneylidene)carbamate 11 (80 mg, 0.153 mmol) in DCM (3 mL) was added TFA (0.3 mL) at room temperature. The solution was stirred at room temperature for 2 hours then concentrated under vacuum. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 55% to 90% MeCN/H₂O containing 0.1% formic acid) to obtain 2-(4,4-difluoroazepan-1-yl)-4-methyl-N-(3-(S-methylsulfonimidoyl)phenyl)nicotinamide (22 mg, 34.38%) as a white solid. ¹H NMR (400 MHz, DMSO-d₆, ppm) δ 10.78 (s, 1H), 8.37 (s, 1H), 8.08 (d, J=4.9 Hz, 1H), 7.91 (d, J=8.4 Hz, 1H), 7.72-7.55 (m, 2H), 6.69 (d, J=5.0 Hz, 1H), 3.63-3.57 (m, 2H), 3.52 (t, J=6.1 Hz, 2H), 3.13 (s, 3H), 2.30-2.18 (m, 5H), 2.06-1.93 (m, 2H), 1.84-1.73 (m, 2H). LCMS (ESI) calcd. for C₂₀H₂₅F₂N₄O₂S [M+H]⁺ m/z 423.17, found 423.05.

### Example 106

### 5-chloro-2-(4,4-difluoroazepan-1-yl)-4,6-dimethyl-N-(3-(S-methylsulfonimidoyl)phenyl)nicotinamide

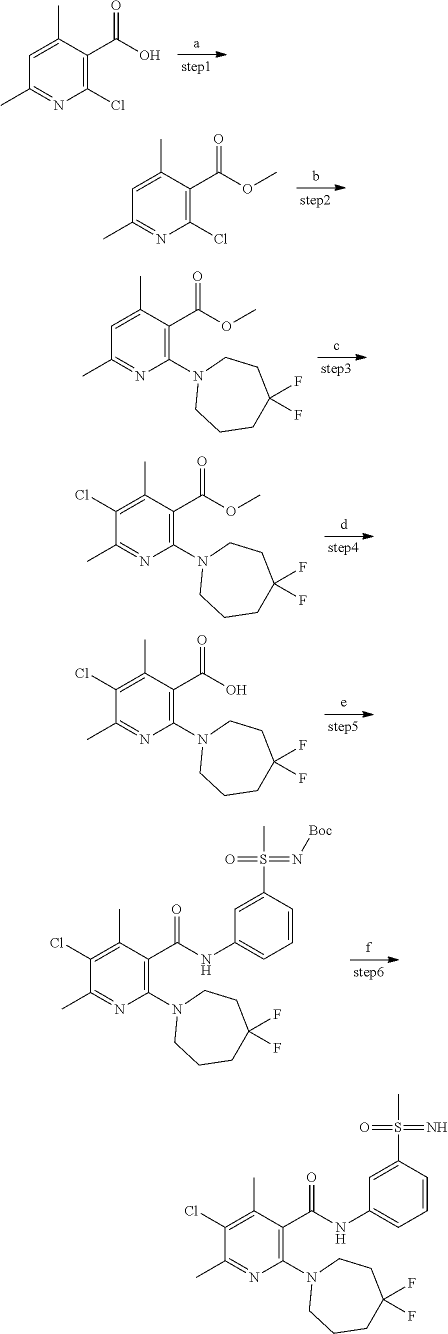

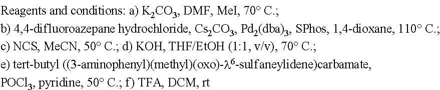

Step 1: methyl 2-chloro-4,6-dimethylnicotinate: To a solution of 2-chloro-4,6-dimethylnicotinic acid (2 g, 10.81 mmol) and K₂CO₃ (4.48 g, 32.43 mmol) in DMF (20 mL) was added Mel(3.07 g, 21.62 mmol) dropwise. Then the mixture was heated at 70° C. for 16 hours. LCMS showed the reaction was completed. The mixture was diluted with water, extracted with EtOAc (3×20 mL). The combined organic phases were washed with brine, dried with Na₂SO₄, and concentrated under vacuum. The residue was purified by silica gel column chromatography (eluting with EtOAc/PE, 0 to 10%) to give methyl 2-(4,4-difluoroazepan-1-yl)-4,6-dimethylnicotinate (1.6 g, 74.38%) as a colorless oil. LCMS (ESI) calcd. for C₉H₁₁ClNO₂ [M+H]⁺ m/z 200.05, found 200.10.

Step 2: methyl 2-(4,4-difluoroazepan-1-yl)-4,6-dimethylnicotinate: A solution of methyl 2-chloro-4,6-dimethylnicotinate (1 g, 5.03 mmol), 4,4-difluoroazepane hydrochloride (1.29 g, 7.54 mmol), Cs₂CO₃ (8.17 g, 25.15 mmol), Pd₂(dba)₃ (0.46 g, 0.50 mmol) and SPhos (0.41 g, 1.00 mmol) in dioxane (10 mL) was stirred at 110° C. for 12 h under N₂ atmosphere. Then the mixture was diluted with water and extracted with EtOAc (3×20 mL). The combined organic phases were washed with brine, dried with Na₂SO₄, and concentrated under vacuum. The residue was purified by silica gel column chromatography (eluting with EtOAc/PE, 0 to 20%) to give methyl 2-(4,4-difluoroazepan-1-yl)-4,6-dimethylnicotinate (0.4 g, 26.64%) as a yellow solid. LCMS (ESI) calcd. for C₁₅H₂₁F₂N₂O₂ [M+H]⁺ m/z 299.16, found 299.15.

Step 3: methyl 5-chloro-2-(4,4-difluoroazepan-1-yl)-4,6-dimethylnicotinate: A solution of methyl 2-(4,4-difluoroazepan-1-yl)-4,6-dimethylnicotinate (400 mg, 1.34 mmol) and NCS (270 mg, 2.01 mmol) in MeCN (5 mL) was heated at 60° C. for 12 h. Then the mixture was diluted with water, extracted with EtOAc (3×10 mL). The combined organic phases were washed with brine, dried with Na₂SO₄, and concentrated under vacuum. The residue was purified by silica gel column chromatography (eluting with EtOAc/PE, 0 to 20%) to give methyl 5-chloro-2-(4,4-difluoroazepan-1-yl)-4,6-dimethylnicotinate (260 mg, 58.44%) as a yellow solid. LCMS (ESI) calcd. for C₁₅H₂₀ClF₂N₂O₂ [M+H]⁺ m/z 333.12, found 333.00.

Step 4: 5-chloro-2-(4,4-difluoroazepan-1-yl)-4,6-dimethylnicotinic acid: To a solution of methyl 5-chloro-2-(4,4-difluoroazepan-1-yl)-4,6-dimethylnicotinate (260 mg, 0.78 mmol) in THF/EtOH (1/1, 4 mL) was added KOH (874 mg, 15.60 mmol) at room temperature. The mixture was heated at 70° C. for 6 hours. After the reaction was completed, the mixture was cooled to room temperature and concentrated to remove THE and EtOH. The residue was dissolved in H₂O (4 mL) and adjusted to pH=3-4 with 1N HCl and extracted with EtOAc (5 mL×3). The combined organic phases were washed with brine, dried with Na₂SO₄, and concentrated under reduced pressure to give 5-chloro-2-(4,4-difluoroazepan-1-yl)-4,6-dimethylnicotinic acid (200 mg, 80.63%). LCMS (ESI) calcd. for C₁₄H₁₈ClF₂N₂O₂ [M+H]⁺ m/z 319.10, found 319.10.

Step 5: tert-butyl ((3-(5-chloro-2-(4,4-difluoroazepan-1-yl)-4,6-dimethylnicotinamido)phenyl)(methyl)(oxo)-λ⁶-sulfaneylidene)carbamate: A mixture of 5-chloro-2-(4,4-difluoroazepan-1-yl)-4,6-dimethylnicotinic acid (200 mg, 0.63 mmol) and tert-butyl ((3-aminophenyl)(methyl)(oxo)-λ⁶-sulfaneylidene)carbamate (255 mg, 0.95 mmol) in pyridine (10 mL) was added POCl₃ (1 mL) dropwise at 50° C. The reaction solution was stirred at 50° C. for 1 hour. After the reaction was completed, the resulting solution was diluted with water (20 mL) and extracted with EtOAc (20 mL×3). The combined organic phases were washed with brine, dried over sodium sulfate, concentrated under vacuum. The residue was purified by flash column chromatography on silica gel (PE/EtOAc=2/1) to give tert-butyl ((3-(5-chloro-2-(4,4-difluoroazepan-1-yl)-4,6-dimethylnicotinamido)phenyl)(methyl)(oxo)-λ⁶-sulfaneylidene)carbamate (130 mg, 36%) as a yellow solid. LCMS (ESI) calcd. for C₂₆H₃₄ClF₂N₄O₄S [M+H]⁺ m/z 571.19, found 571.20.

Step 6: 5-chloro-2-(4,4-difluoroazepan-1-yl)-4,6-dimethyl-N-(3-(S-methylsulfonimidoyl)phenyl)nicotinamide: A solution of tert-butyl ((3-(5-chloro-2-(4,4-difluoroazepan-1-yl)-4,6-dimethylnicotinamido)phenyl)(methyl)(oxo)-λ⁶-sulfaneylidene)carbamate (130 mg, 0.228 mmol) in DCM (2 mL) was added TFA (0.2 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (10 mL×3). The combined organic phases were washed with brine, dried over sodium sulfate, concentrated under vacuum. The residue was purified by prep-HIPLC (Gemini 5 um C₁₈ column, 150*21.2 mm, eluting with 50% to 95% MeCN/H₂O containing 0.05% NH₃) to afford 5-chloro-2-(4,4-difluoroazepan-1-yl)-4,6-dimethyl-N-(3-(S-methylsulfonimidoyl)phenyl)nicotinamide (40 mg, 37.33%) as a white solid. LCMS (ESI) calcd. for C₂₁H₂₆ClF₂N₄O₂S [M+H]⁺ m/z 471.14, found 471.15. ¹H NMR (400 MHz, DMSO-d₆) δ 10.81 (s, 1H), 8.32 (t, J=1.8 Hz, 1H), 7.88 (d, J=8.8 Hz, 1H), 7.62 (m, J=15.8, 8.0 Hz, 2H), 4.23 (s, 1H), 3.56 (m, J=31.8, 8.7, 4.4 Hz, 4H), 3.05 (s, 3H), 2.46 (s, 3H), 2.26 (d, J=8.9 Hz, 5H), 2.03-1.92 (m, 2H), 1.84-1.73 (m, 2H).

### Example 107

### 2-(4,4-difluoroazepan-1-yl)-6-methyl-N-(2-(N-methylsulfamidimidoyl) pyridine-4-yl)-5-(trifluoromethyl)nicotinamide

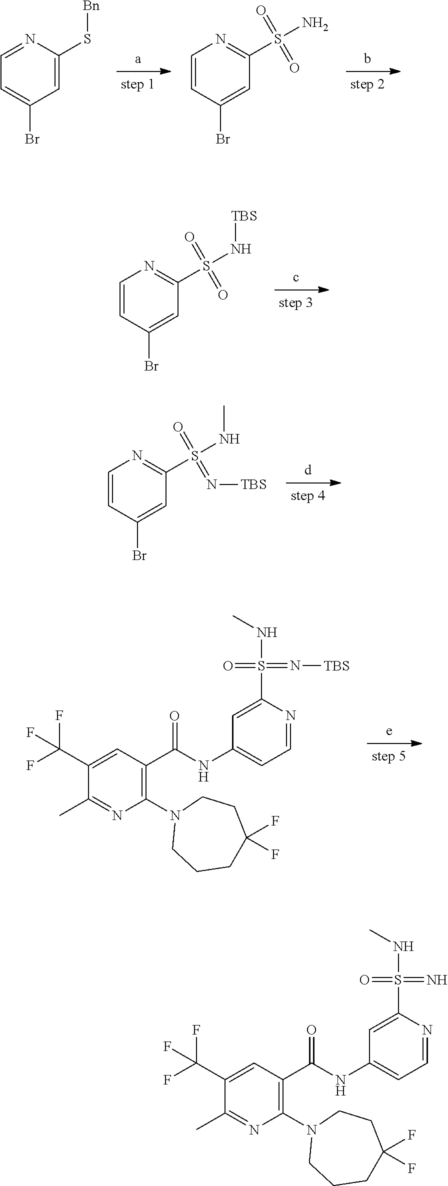

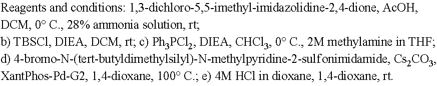

Step 1: 4-bromopyridine-2-sulfonamide: To a stirred solution of 2-(benzylthio)-4-bromopyridine (5.0 g, 17.9 mmoL) in DCM (60 mL), AcOH (8.6 mL) and water (17 mL) was added 1,3-dichloro-5,5-imethyl-imidazolidine-2,4-dione (10.5 g, 53.7 mmol) slowly at 0° C. The mixture was stirred at room temperature for overnight. Then the mixture was diluted with water and extracted with DCM. The combined organic layers were washed with brine, dried over anhydrous magnesium sulfate, filtered, and concentrated under reduced pressure to give the crude sulfonyl chloride. The crude sulfonyl chloride was dissolved in THE (20 mL) and added dropwise to stirred 28% ammonia (20 mL). The solution was stirred at room temperature for 30 min. Then the mixture was diluted with water (40 mL) and extracted with DCM (20 mL×3). The combined organic phases were washed with brine, dried over sodium sulfate, and concentrated under vacuum to give the crude 4-bromopyridine-2-sulfonamide (5 g) which was used directly in next step without further purification. LCMS (ESI) calcd. for C₅H₆BrN₂O₂S [M+H]⁺ m/z 236.93, found 236.90.

Step 2: 4-bromo-N-(tert-butyldimethylsilyl)pyridine-2-sulfonamide: To a solution of 4-bromopyridine-2-sulfonamide (5.0 g, 18.9 mmol) and DIEA (3.67 g, 28.4 mmol) in DCM (30 mL) was added TBSCl (3.4 g, 22.7 mmol) at room temperature. The solution as stirred at room temperature for 3 hours. Then the mixture was diluted with water (40 mL) and extracted with DCM (40 mL×3). The combined organic phases were washed with brine, dried over sodium sulfate, and concentrated under vacuum. The residue was purified by flash column chromatography on silica gel (PE/EtOAc=10/1 to 5/1) to give 4-bromo-N-(tert-butyldimethylsilyl)pyridine-2-sulfonamide (4.5 g, 67%) as a white solid. LCMS (ESI) calcd. for C₁₁H₂₀BrN₂O₂SSi [M+H]⁺ m/z 351.02, found 351.00.

Step 3: 4-bromo-N [symbol] (tert-butyldimethylsilyl)-N-methylpyridine-2-sulfonimidamide: To a 500 mL 3-necked round-bottom flask purged and maintained with an inert atmosphere of nitrogen, was added a solution of Ph₃PCl₂ (3.1 g, 9.3 mmol) in chloroform (40 mL). This was followed by the addition of DIEA (1.88 g, 18.6 mmol) dropwise with stirring at room temperature. The resulting solution was stirred for 10 min at room temperature, then the reaction solution was cooled to 0° C. To this ice-cold solution was added a solution of 4-bromo-N-(tert-butyldimethylsilyl)pyridine-2-sulfonamide (814 mg, 2.32 mmol) in chloroform (8 mL) dropwise at 0° C. The resulting solution was stirred for 30 min at 0° C. Then methylamine (10 mL, 2M in THF) was added at the same temperature. The solution was stirred for 1 hour at room temperature. The resulting solution was diluted with water (40 mL) and extracted with DCM (40 mL×3). The combined organic layers were dried over anhydrous sodium sulfate and concentrated under vacuum. The residue was purified by flash column chromatography on silica gel (PE/EtOAc=10/1) to give 4-bromo-N [symbol] (tert-butyldimethylsilyl)-N-methylpyridine-2-sulfonimidamide (450 mg, 53.4%) as a white solid. LCMS (ESI) calcd. for C₁₂H₂₃BrN₃OSSi [M+H]⁺ m/z 364.05, found 363.90.

Step 4: N-(2-(N [symbol] (tert-butyldimethylsilyl)-N-methylsulfamidimidoyl)pyridin-4-yl)-2-(4,4-difluoroazepan-1-yl)-6-methyl-5-(trifluoromethyl)nicotinamide: A solution of 2-(4,4-difluoroazepan-1-yl)-6-methyl-5-(trifluoromethyl)nicotinamide (125 mg, 0.371 mmol) in dioxane (10 mL) was 4-bromo-N [symbol] tert-butyldimethylsilyl)-N-methylpyridine-2-sulfonimidamide (202 mg, 0.556 mmol), cesium carbonate (314 mg, 0.9644 mmol) and XantPhos-Pd-G2 (66 mg, 0.074 mmol) at room temperature. The reaction mixture was heated at 100° C. for 16 hours under nitrogen. After the reaction was completed, the mixture was diluted with water (20 mL) and extracted with DCM (20 mL×3). The combined organic phases were washed with brine, dried over sodium sulfate, concentrated under vacuum. The residue was purified by flash column chromatography on silica gel (PE/EtOAc=1/1) to give N-(2-(N [symbol] (tert-butyldimethylsilyl)-N-methylsulfamidimidoyl)pyridin-4-yl)-2-(4,4-difluoroazepan-1-yl)-6-methyl-5-(trifluoromethyl)nicotinamide (60 mg, 26.08%) as a yellow oil. LCMS (ESI) calcd. for C₂₆H₃₈F5N₆O₂SSi [M+H]⁺ m/z 621.24, found 621.20.

Step 5: 2-(4,4-difluoroazepan-1-yl)-6-methyl-N-(2-(N-methylsulfamidimidoyl) pyridine-4-yl)-5-(trifluoromethyl)nicotinamide: To a solution of N-(2-(N [symbol] (tert-butyldimethylsilyl)-N-methylsulfamidimidoyl)pyridin-4-yl)-2-(4,4-difluoroazepan-1-yl)-6-methyl-5-(trifluoromethyl)nicotinamide (60 mg, 0.097 mmol) in 1,4-dioxane (5 mL) was added 4M HCl-dioxane (5 mL) at room temperature. The reaction mixture was stirred at room temperature for 1 hour. After the reaction was completed, the mixture was diluted with water (20 mL) and extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 35% to 95% MeCN/H₂O containing 0.1% formic acid) to afford 2-(4,4-difluoroazepan-1-yl)-6-methyl-N-(2-(N-methylsulfamidimidoyl)pyridin-4-yl)-5-(trifluoromethyl)nicotinamide (21.5 mg, 44.79%) as a white solid. ¹H NMR (400 MHz, DMSO-d₆) δ 11.09 (bs, 1H), 8.49 (s, 1H), 8.24 (s, 1H), 7.97 (s, 1H), 7.78 (s, 1H), 6.83 (bs, 1H), 4.21 (bs, 1H), 3.69-3.68 (m, 2H), 3.33-3.32 (m, 2H), 2.49 (s, 3H), 2.47 (s, 3H), 2.32-2.30 (m, 2H), 1.95-1.91 (m, 2H), 1.87-1.86 (m, 2H). LCMS (ESI) calcd. for C₂₀H₂₄F₅N₆O₂S [M+H]⁺ m/z 507.16, found 507.10.

### Example 108

### (S)-5-chloro-2-(4,4-difluoroazepan-1-yl)-4-methyl-N-(3-(S-methylsulfonimidoyl)phenyl)-6-(trifluoromethyl)nicotinamide

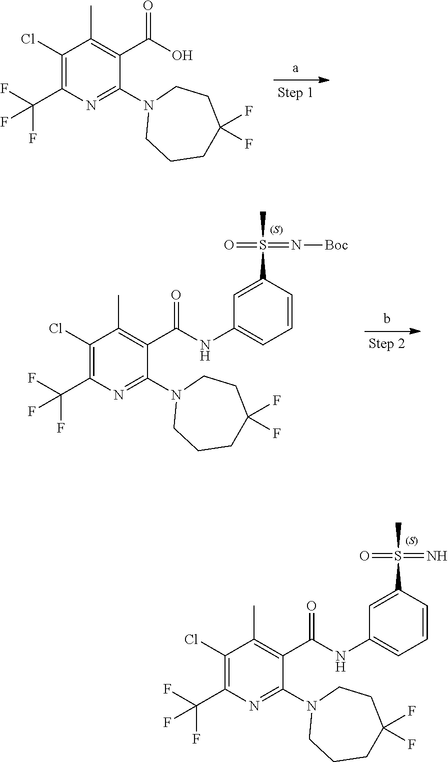

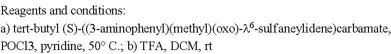

Step 1: tert-butyl (S)-((3-(5-chloro-2-(4,4-difluoroazepan-1-yl)-4-methyl-6-(trifluoromethyl)nicotinamido)phenyl)(methyl)(oxo)-λ⁶-sulfaneylidene)carbamate: A mixture of 5-chloro-2-(4,4-difluoroazepan-1-yl)-4-methyl-6-(trifluoromethyl)nicotinic acid (250 mg, 0.676 mmol) and tert-butyl (S)-((3-aminophenyl)(methyl)(oxo)-λ⁶-sulfaneylidene)carbamate (274 mg, 1.013 mmol) in pyridine (15 mL) was added POCl₃ (450 L) at 50° C. After the reaction was completed, the mixture was diluted with water (15 mL) and extracted with DCM (15 mL×3). The combined organic phases were washed with brine, dried over Na₂SO₄ and concentrated under vacuum. The residue was purified by flash column chromatography on silica gel (PE/EtAOc=1/1) to give tert-butyl (S)-((3-(5-chloro-2-(4,4-difluoroazepan-1-yl)-4-methyl-6-(trifluoromethyl)nicotinamido)phenyl)(methyl)(oxo)-λ⁶-sulfaneylidene)carbamate (40 mg, 9.47%) as a yellow oil. LCMS (ESI) calcd. for C₂₆H₂₉ClF₅N₄O₄S [M−H]⁻ m/z 623.15, found 623.35.

Step 2: (S)-5-chloro-2-(4,4-difluoroazepan-1-yl)-4-methyl-N-(3-(S-methylsulfonimidoyl)phenyl)-6-(trifluoromethyl)nicotinamide: A solution of tert-butyl (S)-((3-(5-chloro-2-(4,4-difluoroazepan-1-yl)-4-methyl-6-(trifluoromethyl)nicotinamido)phenyl)(methyl)(oxo)-λ⁶-sulfaneylidene)carbamate (40 mg, 0.064 mmol) in DCM (3 mL) was added TFA (1 mL) at room temperature. The reaction mixture was stirred at room temperature for 2 hours. After the reaction was completed, the solution was diluted with water (10 mL) and extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 50% to 80% MeCN/H₂O containing 0.1% formic acid) to (S)-5-chloro-2-(4,4-difluoroazepan-1-yl)-4-methyl-N-(3-(S-methylsulfonimidoyl)phenyl)-6-(trifluoromethyl)nicotinamide (12.5 mg, 37.87%) as a white solid. ¹H NMR (400 MHz, DMSO-d₆) δ 10.98 (s, 1H), 8.28 (s, 1H), 7.88 (d, J=7.9 Hz, 1H), 7.70 (d, J=7.3 Hz, 1H), 7.61 (t, J=7.7 Hz, 1H), 4.24 (s, 1H), 3.65 (s, 2H), 3.58 (t, J=6.0 Hz, 2H), 3.06 (s, 3H), 2.36 (s, 3H), 2.31 (d, J=18.2 Hz, 2H), 2.08-1.95 (m, 2H), 1.86-1.77 (m, 2H). LCMS (ESI) calcd. for C₂₁H₂₃ClF₅N₄O₂S [M+H]⁺ m/z 525.11, found 525.15.

### Example 109

### (R)-5-chloro-2-(4,4-difluoroazepan-1-yl)-4-methyl-N-(3-(S-methylsulfonimidoyl)phenyl)-6-(trifluoromethyl)nicotinamide

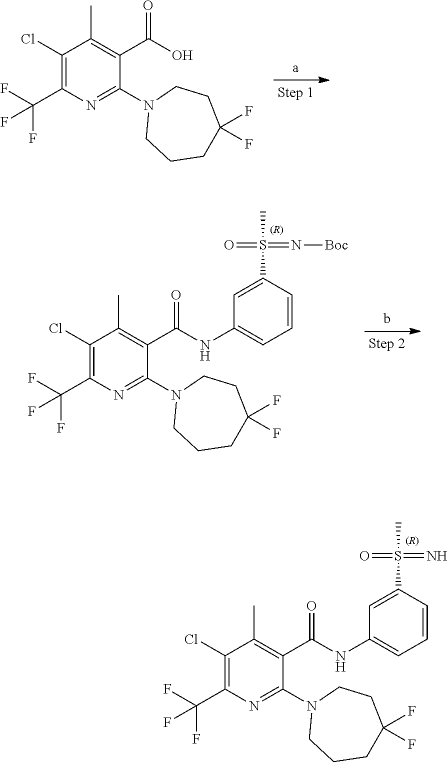

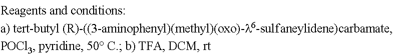

Step 1: tert-butyl (R)-((3-(5-chloro-2-(4,4-difluoroazepan-1-yl)-4-methyl-6-(trifluoromethyl)nicotinamido)phenyl)(methyl)(oxo)-λ⁶-sulfaneylidene)carbamate: A mixture of 5-chloro-2-(4,4-difluoroazepan-1-yl)-4-methyl-6-(trifluoromethyl)nicotinic acid (250 mg, 0.6756 mmol) and tert-butyl (R)-((3-aminophenyl)(methyl)(oxo)-λ⁶-sulfaneylidene)carbamate (274 mg, 1.0134 mmol) in pyridine (15 mL) was added POCl₃ (450 L) at 50° C. After the reaction was completed, the mixture was cooled to room temperature. The resulting solution was diluted with water (25 mL) and extracted with DCM (25 mL×3). The combined organic phases were washed with brine, dried over Na₂SO₄, concentrated under vacuum. The residue was purified by flash column chromatography on silica gel (PE/EtAOc=1/1) to give tert-butyl (R)-((3-(5-chloro-2-(4,4-difluoroazepan-1-yl)-4-methyl-6-(trifluoromethyl)nicotinamido)phenyl)(methyl)(oxo)-λ⁶-sulfaneylidene)carbamate (40 mg, 9.47%) as a yellow oil. LCMS (ESI) calcd. for C₂₆H₃₁ClF₅N₄O₄S [M+H]⁺ m/z 625.17, found 525.10.

Step 2: (R)-5-chloro-2-(4,4-difluoroazepan-1-yl)-4-methyl-N-(3-(S-methylsulfonimidoyl)phenyl)-6-(trifluoromethyl)nicotinamide: A solution of tert-butyl (R)-((3-(5-chloro-2-(4,4-difluoroazepan-1-yl)-4-methyl-6-(trifluoromethyl)nicotinamido)phenyl)(methyl)(oxo)-λ⁶-sulfaneylidene)carbamate (40 mg, 0.0641 mmol) in DCM (3 mL) was added TFA (1 mL) at room temperature. The reaction mixture was stirred at room temperature for 2 hours. After the reaction was completed, the solution was diluted with water (10 mL) and extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 50% to 80% MeCN/H₂O containing 0.1% formic acid) to (R)-5-chloro-2-(4,4-difluoroazepan-1-yl)-4-methyl-N-(3-(S-methylsulfonimidoyl)phenyl)-6-(trifluoromethyl)nicotinamide (7.5 mg, 27.87%) as a white solid. ¹H NMR (400 MHz, DMSO-d₆) δ 10.98 (s, 1H), 8.29 (s, 1H), 7.89 (d, J=8.2 Hz, 1H), 7.70 (d, J=7.9 Hz, 1H), 7.61 (t, J=7.9 Hz, 1H), 4.25 (s, 1H), 3.65 (s, 2H), 3.58 (t, J=6.1 Hz, 2H), 3.07 (s, 3H), 2.36 (s, 3H), 2.33-2.28 (m, 2H), 2.05-1.98 (m, 2H), 1.82 (d, J=6.0 Hz, 2H). LCMS (ESI) calcd. for C₂₁H₂₃ClF₅N₄O₂S [M+H]⁺ m/z 525.11, found 525.05.

### Example 110

### (R)-2-(4,4-difluoroazepan-1-yl)-5-(3-fluorophenyl)-4-methyl-N-(3-(S-methylsulfonimidoyl)phenyl)nicotinamide

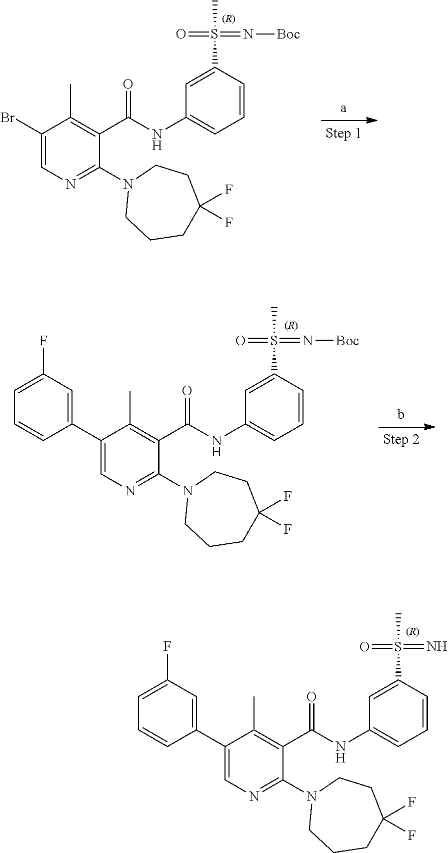

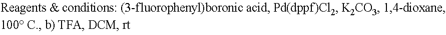

Step 1: tert-butyl (R)-((3-(2-(4,4-difluoroazepan-1-yl)-5-(3-fluorophenyl)-4-methylnicotinamido)phenyl)(methyl)(oxo)-λ⁶-sulfaneylidene)carbamate: A mixture of tert-butyl (R)-((3-(5-bromo-2-(4,4-difluoroazepan-1-yl)-4-methylnicotinamido)phenyl)(methyl)(oxo)-λ⁶-sulfaneylidene)carbamate (70 mg, 0.117 mmol), (3-fluorophenyl)boronic acid (49.0 mg, 0.35 mmol), potassium carbonate (47.3 mg, 0.35 mmol) and [1,1′-Bis(diphenylphosphino)ferrocene]dichloropalladium (II) (17.1 mg, 0.0234 mmol) in 1,4-dioxane/H₂O (4/1, 4 mL) was heated at 100° C. for 16 h under an atmosphere of N₂. After the reaction was completed, the mixture was diluted with water (30 mL) and extracted with EtOAc (30 mL×3). The combined organic phases were washed with brine, dried over Na₂SO₄, concentrated under vacuum. The residue was purified by flash column chromatography on silica gel (PE/EtAOc=1/1) to give tert-butyl (R)-((3-(2-(4,4-difluoroazepan-1-yl)-5-(3-fluorophenyl)-4-methylnicotinamido)phenyl)(methyl)(oxo)-λ⁶-sulfaneylidene)carbamate (60 mg, 83.15%) as a white oil. LCMS (ESI) calcd. for C₃₁H₃₆F₃N₄O₄S [M+H]⁺ m/z 617.24, found 617.20.

Step 2: (R)-2-(4,4-difluoroazepan-1-yl)-5-(3-fluorophenyl)-4-methyl-N-(3-(S-methylsulfonimidoyl)phenyl)nicotinamide: A solution of tert-butyl (R)-((3-(2-(4,4-difluoroazepan-1-yl)-5-(3-fluorophenyl)-4-methylnicotinamido)phenyl)(methyl)(oxo)-λ⁶-sulfaneylidene)carbamate (60 mg, 0.0974 mmol) in DCM (3 mL) was added TFA (0.5 mL) at room temperature. The reaction mixture was stirred at room temperature for 2 hours. Then the mixture was concentrated. The residue was adjusted to pH=8-9 with saturated aqueous NaHCO₃. The aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 50% to 90% MeCN/H₂O containing 0.05% NH₃) to provide (R)-2-(4,4-difluoroazepan-1-yl)-5-(3-fluorophenyl)-4-methyl-N-(3-(S-methylsulfonimidoyl)phenyl)nicotinamide (11.2 mg, 22.5%) as a white solid. ¹H NMR (400 MHz, DMSO-d₆) δ 10.88 (s, 1H), 8.38 (s, 1H), 8.11 (s, 1H), 7.90 (d, J=8.1 Hz, 1H), 7.67 (d, J=7.9 Hz, 1H), 7.58 (t, J=7.9 Hz, 1H), 7.51 (dd, J=14.3, 7.9 Hz, 1H), 7.25-7.17 (m, 3H), 4.23 (s, 1H), 3.66 (dd, J=6.5, 3.6 Hz, 2H), 3.59 (t, J=6.1 Hz, 2H), 3.06 (s, 3H), 2.35-2.23 (m, 2H), 2.17 (s, 3H), 2.09-1.96 (m, 2H), 1.83 (d, J=5.7 Hz, 2H). LCMS (ESI) calcd. for C₂₆H₂₈F₃N₄O₂S [M+H]⁺ m/z 517.19, found 517.15

### Example 111

### (S)-2-(4,4-difluoroazepan-1-yl)-5-(3-fluorophenyl)-4-methyl-N-(3-(S-methylsulfonimidoyl)phenyl)nicotinamide

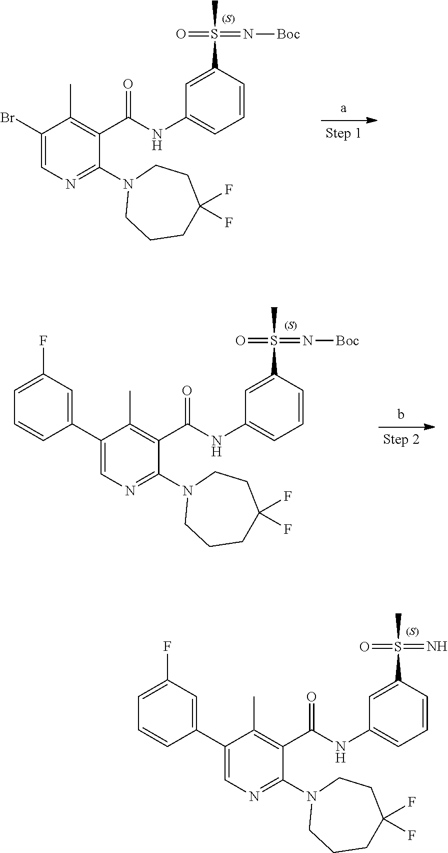

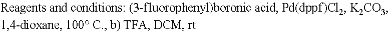

Step 1: tert-butyl (S)-((3-(2-(4,4-difluoroazepan-1-yl)-5-(3-fluorophenyl)-4-methylnicotinamido)phenyl)(methyl)(oxo)-λ⁶-sulfaneylidene)carbamate: A mixture of tert-butyl (S)-((3-(5-bromo-2-(4,4-difluoroazepan-1-yl)-4-methylnicotinamido)phenyl)(methyl)(oxo)-λ⁶-sulfaneylidene)carbamate (100 mg, 0.167 mmol), (3-fluorophenyl)boronic acid (117 mg, 0.833 mmol), potassium carbonate (69 mg, 0.501 mmol) and [1,1′-Bis(diphenylphosphino)ferrocene]dichloropalladium (II) (13 mg, 0.0167 mmol) in 1,4-dioxane/H₂O (4/1, 10 mL) was heated at 100° C. for 6 hours under an atmosphere of N₂. The reaction was monitored by LCMS. After the reaction was completed, the mixture was diluted with water (20 mL) and extracted with DCM (20 mL×3). The combined organic phases were washed with brine, dried over Na₂SO₄, concentrated under vacuum. The residue was purified by flash column chromatography on silica gel (PE/EtAOc=1/1) to give tert-butyl (S)-((3-(2-(4,4-difluoroazepan-1-yl)-5-(3-fluorophenyl)-4-methylnicotinamido)phenyl)(methyl)(oxo)-λ⁶-sulfaneylidene)carbamate (68 mg, 66.67%) as a yellow oil. LCMS (ESI) calcd. for C₃₁H₃₆F₃N₄O₄S [M+H]⁺ m/z 617.24, found 617.20.

Step 2: (S)-2-(4,4-difluoroazepan-1-yl)-5-(3-fluorophenyl)-4-methyl-N-(3-(S-methylsulfonimidoyl)phenyl)nicotinamide: A solution of tert-butyl (S)-((3-(2-(4,4-difluoroazepan-1-yl)-5-(3-fluorophenyl)-4-methylnicotinamido)phenyl)(methyl)(oxo)-λ⁶-sulfaneylidene)carbamate (68 mg, 0.110 mmol) in DCM (5 mL) was added TFA (0.5 mL) at room temperature. The reaction mixture was stirred at room temperature for 2 hours. After the reaction was completed, the solution was diluted with water (10 mL) and extracted with DCM (10 mL×3). The combined organic phases were washed with brine, dried over sodium sulfate, and concentrated under vacuum. The residue was purified by prep-HPLC (Gemini 5 um C_{18 column,} 150*21.2 mm, eluting with 40% to 90% MeCN/H₂O containing 0.1% formic acid) to obtain (S)-2-(4,4-difluoroazepan-1-yl)-5-(3-fluorophenyl)-4-methyl-N-(3-(S-methylsulfonimidoyl)phenyl)nicotinamide (12.5 mg, 22.32%) as a white solid. ¹H NMR (400 MHz, DMSO-d₆, ppm) δ 10.88 (s, 1H), 8.38-8.36 (m, 1H), 8.10 (s, 1H), 7.90 (d, J=8.8 Hz, 1H), 7.66 (d, J=8 Hz, 1H), 7.58-7.56 (m, 1H), 7.51-7.47 (m, 1H), 7.24-7.17 (m, 3H), 4.22 (s, 1H), 3.67-3.65 (m, 2H), 3.60-3.57 (m, 2H), 3.06 (s, 3H), 2.32-2.26 (m, 2H), 2.14 (s, 3H), 2.07-1.96 (m, 2H), 1.83-1.82 (m, 2H). LCMS (ESI) calcd. for C₂₆H₂₈F₃N₄O₂S [M+H]⁺ m/z 517.19, found 517.10.

### Example 112

### 6(4,4-difluoroazepan-1-yl)-5-fluoro-4methyl-N-(3-(S-methylsulfonimidoyl)phenyl)-[2,3bipyridine]-5carboxamide

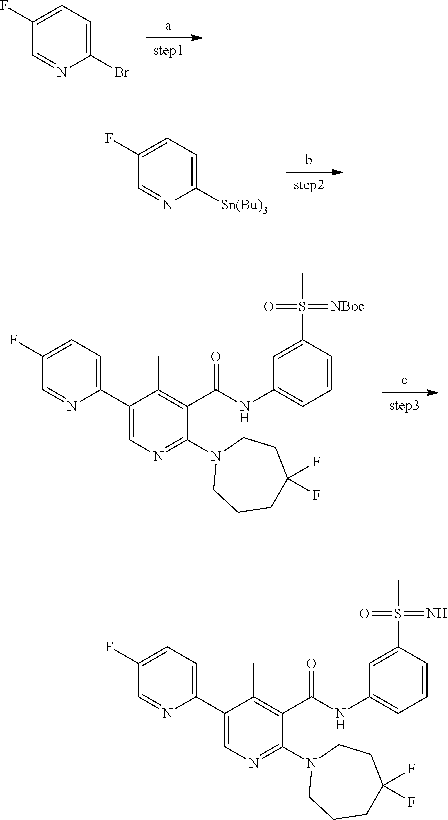

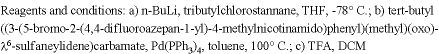

Step 1: 5-fluoro-2-(tributylstannyl)pyridine: To a mixture of 2-bromo-5-fluoropyridine (1 g, 5.714 mmol) in THE (15 mL) was added n-BuLi (2.86 mL, 6.587 mmol) dropwise at −78° C. The mixture was stirred at −78° C. for 0.5 h. Then tributylchlorostannane (2.15 g, 6.587 mmol) was added to the mixture at −78° C. The mixture was stirred at room temperature for 2 hours under an atmosphere of N₂. After the reaction was completed, the mixture was quenched with saturated aqueous ammonium chloride (30 mL) and extracted with EtOAc (20 mL×3). The combined organic phases were washed with brine, dried over Na₂SO₄, concentrated under vacuum. The residue was purified by flash column chromatography on silica gel (PE/EtAOc=10/1) to give 5-fluoro-2-(tributylstannyl)pyridine (1.1 g, 49.77%) as a colorless oil. LCMS (ESI) calcd. for C₁₇H₃₁FNSn [M+H]⁺ m/z 388.14, found 386.05.

Step 2: tert-butyl ((3-(6 [symbol] (4,4-difluoroazepan-1-yl)-5-fluoro-4 [symbol] methyl-[2,3 [symbol] bipyridine]-5 [symbol] carboxamido)phenyl)(methyl)(oxo)-λ⁶-sulfaneylidene)carbamate: A mixture of tert-butyl ((3-(5-bromo-2-(4,4-difluoroazepan-1-yl)-4-methylnicotinamido)phenyl)(methyl)(oxo)-λ⁶-sulfaneylidene)carbamate (100 mg, 0.167 mmol), 5-fluoro-2-(tributylstannyl)pyridine (389 mg, 1.002 mmol), Pd(PPh₃)₄ (19 mg, 0.017 mmol) in toluene (10 mL) was heated at 100° C. for 16 hours under an atmosphere of N₂. After the reaction was completed, the mixture was concentrated under vacuum. The residue was purified by flash column chromatography on silica gel (PE/EtAOc=1/1) to give tert-butyl ((3-(6 [symbol] (4,4-difluoroazepan-1-yl)-5-fluoro-4 [symbol] methyl-[2,3 [symbol] bipyridine]-5 [symbol] carboxamido)phenyl)(methyl)(oxo)-λ⁶-sulfaneylidene)carbamate (70 mg, 67.96%) as a yellow oil. LCMS (ESI) calcd. for C₃₀H₃₅F₃N₅O₄S [M+H]⁺ m/z 618.24, found 618.20.

Step 3: 6 [symbol] (4,4-difluoroazepan-1-yl)-5-fluoro-4 [symbol] methyl-N-(3-(S-methylsulfonimidoyl)phenyl)-[2,3 [symbol] bipyridine]-5 [symbol] carboxamide: A solution of tert-butyl ((3-(6 [symbol] (4,4-difluoroazepan-1-yl)-5-fluoro-4 [symbol] methyl-[2,3 [symbol] bipyridine]-5 [symbol] carboxamido)phenyl)(methyl)(oxo)-λ⁶-sulfaneylidene)carbamate (70 mg, 0.113 mmol) in DCM (3 mL) was added TFA (0.3 mL) at room temperature. The solution was stirred at room temperature for 2 hours then concentrated under vacuum. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 55% to 90% MeCN/H₂O containing 0.1% formic acid) to obtain 61-(4,4-difluoroazepan-1-yl)-5-fluoro-4 [symbol] methyl-N-(3-(S-methylsulfonimidoyl)phenyl)-[2,3 [symbol] bipyridine]-5 [symbol] carboxamide (30.4 mg, 51.88%) as a white solid. ¹H NMR (400 MHz, DMSO-d₆) δ 10.94 (s, 1H), 8.73-8.66 (m, 1H), 8.53 (d, J=4.8 Hz, 1H), 8.37 (d, J=1.8 Hz, 1H), 8.14 (d, J=1.8 Hz, 1H), 7.91 (d, J=7.2 Hz, 1H), 7.68 (d, J=7.5 Hz, 1H), 7.59 (td, J=7.9, 2.0 Hz, 1H), 7.52-7.42 (m, 1H), 4.23 (s, 1H), 3.69 (dd, J=5.1, 2.6 Hz, 2H), 3.61 (t, J=5.3 Hz, 2H), 3.06 (d, J=1.2 Hz, 3H), 2.39-2.25 (m, 2H), 2.12-2.07 (m, 3H), 2.06-1.95 (m, 2H), 1.88-1.78 (m, 2H). LCMS (ESI) calcd. for C₂₅H₂₇F₃N₅O₂S [M+H]⁺ m/z 518.19, found 518.10.

### Example 113

### 2-(4,4-difluoroazepan-1-yl)-4-methyl-N-(3-(S-methylsulfonimidoyl)phenyl)-5-(3-methylthiophen-2-yl)nicotinamide

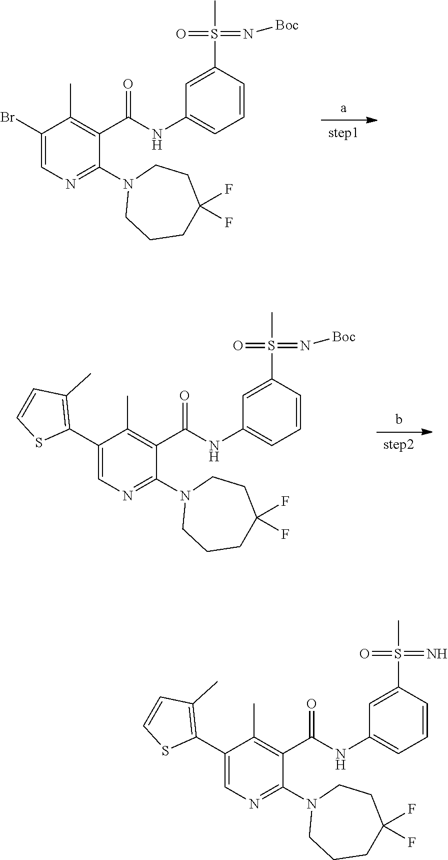 [SMILES: Cc1c(Br)cnc(N2CCCC(F)(F)CC2)c1C(=O)Nc1cccc(S(C)(=O)=NC(=O)OC(C)(C)C)c1 ; Cc1ccsc1-c1cnc(N2CCCC(F)(F)CC2)c(C(=O)Nc2cccc(S(C)(=O)=NC(=O)OC(C)(C)C)c2)c1C ; Cc1ccsc1-c1cnc(N2CCCC(F)(F)CC2)c(C(=O)Nc2cccc(S(C)(=N)=O)c2)c1C]

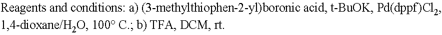

Step 1: tert-butyl ((3-(2-(4,4-difluoroazepan-1-yl)-4-methyl-5-(3-methylthiophen-2-yl)nicotinamido)phenyl)(methyl)(oxo)-λ⁶-sulfaneylidene)carbamate: A mixture of tert-butyl ((3-(5-bromo-2-(4,4-difluoroazepan-1-yl)-4-methylnicotinamido)phenyl)(methyl)(oxo)-λ⁶-sulfaneylidene)carbamate (200 mg, 0.33 mmol), (3-methylthiophen-2-yl)boronic acid (95 mg, 0.66 mmol), t-BuOK (75 mg, 0.66 mmol) and [1,1′-Bis(diphenylphosphino)ferrocene]dichloropalladium (II) (24 mg, 0.033 mmol) in 1,4-dioxane/H₂O (4/1, 10 mL) was heated at 100° C. for 16 hours under an atmosphere of N₂. After the reaction was completed, the mixture was concentrated under vacuum. The residue was purified by flash column chromatography on silica gel (PE/EtAOc=1/2) to give tert-butyl ((3-(2-(4,4-difluoroazepan-1-yl)-4-methyl-5-(3-methylthiophen-2-yl)nicotinamido)phenyl)(methyl)(oxo)-λ⁶-sulfaneylidene)carbamate (100 mg, 49.0%) as a yellow solid. LCMS (ESI) calcd. for C₃₀H₃₇F₂N₄O₄S₂ [M+H]⁺ m/z 619.22, found 619.20.

Step 2: 2-(4,4-difluoroazepan-1-yl)-4-methyl-N-(3-(S-methylsulfonimidoyl)phenyl)-5-(3-methylthiophen-2-yl)nicotinamide: A solution of tert-butyl ((3-(2-(4,4-difluoroazepan-1-yl)-4-methyl-5-(3-methylthiophen-2-yl)nicotinamido)phenyl)(methyl)(oxo)-λ⁶-sulfaneylidene)carbamate (60 mg, 0.09 mmol) in DCM (1.5 mL) was added TFA (0.5 mL) at room temperature. The solution was stirred at room temperature for 2 hours then concentrated under vacuum. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.1% NH₃) to obtain 2-(4,4-difluoroazepan-1-yl)-4-methyl-N-(3-(S-methylsulfonimidoyl)phenyl)-5-(3-methylthiophen-2-yl)nicotinamide (27.9 mg, 99.2%) as a white solid. ¹H NMR (400 MHz, DMSO-d₆) 10.88 (s, 1H), 8.35 (t, J=1.8 Hz, 1H), 8.06 (s, 1H), 7.92 (d, J=8.1 Hz, 1H), 7.67 (d, J=8.1 Hz, 1H), 7.59 (t, J=7.9 Hz, 1H), 7.52 (d, J=5.1 Hz, 1H), 7.03 (d, J=5.1 Hz, 1H), 4.23 (s, 1H), 3.71-3.62 (m, 2H), 3.59 (t, J=6.1 Hz, 2H), 3.06 (s, 3H), 2.29 (t, J=13.2 Hz, 2H), 2.07 (d, J=3.2 Hz, 3H), 2.04-1.96 (m, 5H), 1.86-1.78 (m, 2H). LCMS (ESI) calcd. for C₂₅H₂₉F₂N₄O₂S₂ [M+H]⁺ m/z 519.17, found 519.10.

### Example 114

### 2-(4,4-difluoroazepan-1-yl)-6-methyl-N-(2-sulfamidimidoylpyridin-4-yl)-5-(trifluoromethyl)nicotinamide

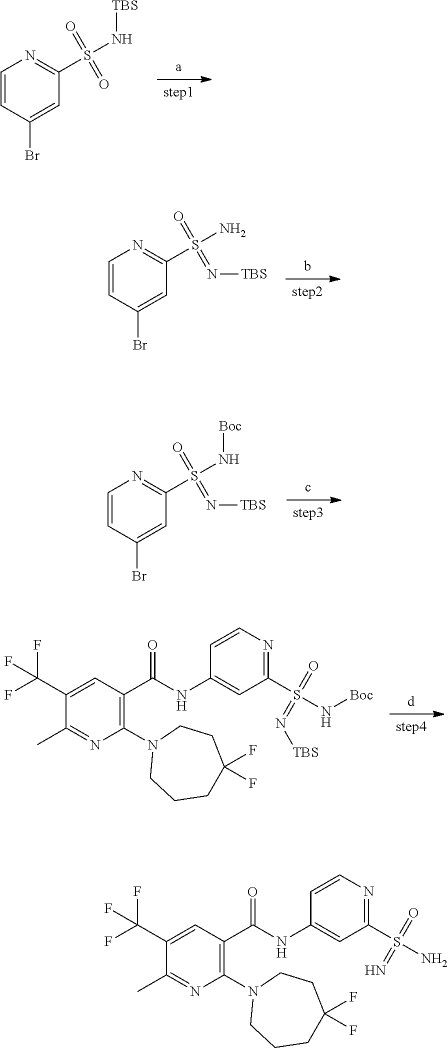

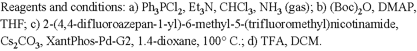

Step 1: 4-bromo-N [symbol] tert-butyldimethylsilyl)pyridine-2-sulfonimidamide: To a 250 mL 3-necked round-bottom flask purged and maintained with an inert atmosphere of nitrogen, was added a solution of Ph₃PCl₂ (1.13 g, 3.4 mmol) in chloroform (24 mL). This was followed by the addition of TEA (0.96 mL) dropwise with stirring at room temperature. The resulting solution was stirred for 10 min at room temperature, then the reaction solution was cooled to 0° C. To this ice-cold solution was added a solution of 4-bromo-N-(tert-butyldimethylsilyl)pyridine-2-sulfonamide (600 mg, 1.7 mmol) in chloroform (8 mL) dropwise at 0° C. The resulting solution was stirred for 30 min at 25° C. And then ammonia gas was introduced into the reaction system. The solution was stirred for 1 hour at room temperature. The resulting solution was diluted with water (40 mL) and extracted with DCM (40 mL×3). The combined organic layers were dried over anhydrous sodium sulfate and concentrated under vacuum. The residue was purified by flash column chromatography on silica gel (PE/EtOAc=2/1) to give 4-bromo-N [symbol] (tert-butyldimethylsilyl)pyridine-2-sulfonimidamide (300 mg, 50.5%) as a white solid. LCMS (ESI) calcd. for C₁₁H₂₁BrN₃OSSi [M+H]⁺ m/z 352.04, found 352.00.

Step 2: tert-butyl (4-bromo-N-(tert-butyldimethylsilyl)pyridine-2-sulfonimidoyl)carbamate: A solution of 4-bromo-N [symbol] tert-butyldimethylsilyl)pyridine-2-sulfonimidamide (300 mg, 0.86 mmol) and DMAP (126 mg, 1.03 mmol) in THE (5 mL) was added di-tert-butyl dicarbonate (750 mg, 3.44 mmol) at room temperature. The solution as stirred at room temperature for 1 hour. After the reaction was completed, the mixture was diluted with water (20 mL) and extracted with DCM (20 mL×3). The combined organic phases were washed with brine, dried over sodium sulfate, concentrated under vacuum. The residue was purified by flash column chromatography on silica gel (PE/EtOAc=2/1) to give tert-butyl (4-bromo-N-(tert-butyldimethylsilyl)pyridine-2-sulfonimidoyl)carbamate (250 mg, 64.7%) as a white solid. LCMS (ESI) calcd. for C₁₆H₂₉BrN₃O₃SSi [M+H]⁺ m/z 450.09, found 450.05.

Step 3: tert-butyl (N-(tert-butyldimethylsilyl)-4-(2-(4,4-difluoroazepan-1-yl)-6-methyl-5-(trifluoromethyl)nicotinamido)pyridine-2-sulfonimidoyl)carbamate: A solution of 2-(4,4-difluoroazepan-1-yl)-6-methyl-5-(trifluoromethyl)nicotinamide (165 mg, 0.49 mmol) in dioxane (10 mL) was added tert-butyl (4-bromo-N-(tert-butyldimethylsilyl)pyridine-2-sulfonimidoyl)carbamate (220 mg, 0.49 mmol), cesium carbonate (415 mg, 1.27 mmol) and XantPhos-Pd-G2 (43.5 mg, 0.049 mmol) at room temperature. The reaction mixture was heated at 100° C. for 16 hours under nitrogen. After the reaction was completed, the mixture was diluted with water (20 mL) and extracted with DCM (20 mL×3). The combined organic phases were washed with brine, dried over sodium sulfate, concentrated under vacuum. The residue was purified by flash column chromatography on silica gel (PE/EtOAc=1/1) to give tert-butyl (N-(tert-butyldimethylsilyl)-4-(2-(4,4-difluoroazepan-1-yl)-6-methyl-5-(trifluoromethyl)nicotinamido)pyridine-2-sulfonimidoyl)carbamate (120 mg, 34.7%) as a yellow oil. LCMS (ESI) calcd. for C₃₀H₄₄F₅N₆O₄SSi [M+H]⁺ m/z 707.28, found 707.35.

Step 4: 2-(4,4-difluoroazepan-1-yl)-6-methyl-N-(2-sulfamidimidoylpyridin-4-yl)-5-(trifluoromethyl)nicotinamide: To a solution of tert-butyl (N-(tert-butyldimethylsilyl)-4-(2-(4,4-difluoroazepan-1-yl)-6-methyl-5-(trifluoromethyl)nicotinamido)pyridine-2-sulfonimidoyl)carbamate (100 mg, 0.14 mmol) in DCM (5 mL) was added TFA (1 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 45% to 95% MeCN/H₂O containing 0.05% NH₃) to afford 2-(4,4-difluoroazepan-1-yl)-6-methyl-N-(2-sulfamidimidoylpyridin-4-yl)-5-(trifluoromethyl)nicotinamide (33.2 mg, 48.2%) as a white solid. ¹H NMR (400 MHz, DMSO-d₆) δ 11.09 (s, 1H), 8.55 (d, J=5.4 Hz, 1H), 8.34 (d, J=1.5 Hz, 1H), 8.00 (s, 1H), 7.82 (dd, J=5.4, 1.8 Hz, 1H), 6.71 (s, 2H), 3.74-3.67 (m, 2H), 3.41 (t, J=5.3 Hz, 2H), 2.52 (s, 3H), 2.33 (s, 2H), 1.94 (d, J=15.4 Hz, 2H), 1.88 (d, J=4.5 Hz, 2H). LCMS (ESI) calcd. for C₁₉H₂₂F₅N₆O₂S [M+H]⁺ m/z 493.15, found 493.10.

### Example 115

### 3-(4,4-difluoroazepan-1-yl)-5-methyl-N-(3-sulfamidimidoylphenyl)-6-(trifluoromethyl)pyridazine-4-carboxamide

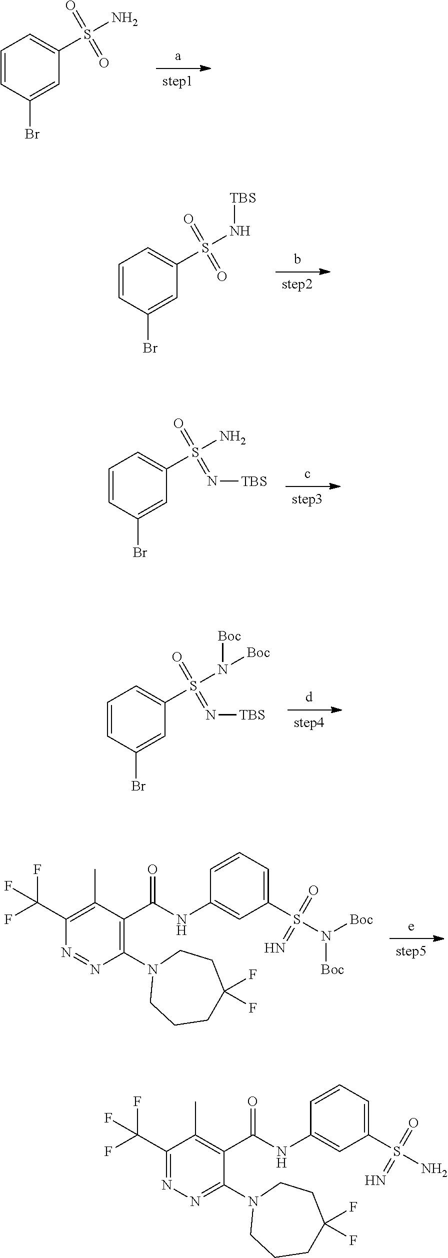

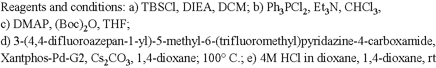

Step 1: 3-bromo-N-(tert-butyldimethylsilyl)benzenesulfonamide: To a solution of 3-bromobenzenesulfonamide (5.0 g, 21.2 mmol) and DIEA (4.11 g, 31.8 mmol) in DCM (30 mL) was added TBSCl (3.84 g, 25.4 mmol) at room temperature. The solution as stirred at room temperature for 3 hours. Then the mixture was diluted with water (40 mL) and extracted with DCM (40 mL×3). The combined organic phases were washed with brine, dried over sodium sulfate, and concentrated under vacuum. The residue was purified by flash column chromatography on silica gel (PE/EtOAc=10/1 to 5/1) to give 3-bromo-N-(tert-butyldimethylsilyl)benzenesulfonamide (4.7 g, 63.5%) as a white solid. ¹H NMR (400 MHz, DMSO-d₆) δ 7.85 (t, J=1.8 Hz, 1H), 7.74-7.66 (m, 3H), 7.45 (t, J=7.9 Hz, 1H), 0.76 (s, 9H), 0.00 (s, 6H)

Step 2: 3-bromo-N [symbol] (tert-butyldimethylsilyl)benzenesulfonimidamide: To a 500 mL 3-necked round-bottom flask purged and maintained with an atmosphere of nitrogen, was added a solution of Ph₃PCl₂ (6 g, 18.1 mmol) in chloroform (40 mL). This was followed by the addition of Et₃N (2.30 g, 22.8 mmol) dropwise with stirring at room temperature. The resulting solution was stirred for 10 min at room temperature, then the reaction solution was cooled to 0° C. To this ice-cold solution was added a solution of 3-bromo-N-(tert-butyldimethylsilyl)benzenesulfonamide (4 g, 11.4 mmol) in chloroform (8 mL) dropwise at 0° C. The resulting solution was stirred for 30 min at 25° C. And then ammonia gas was introduced into the reaction system. The solution was stirred for 1 hour at room temperature. The resulting solution was diluted with water (40 mL) and extracted with DCM (40 mL×3). The combined organic layers were dried over anhydrous sodium sulfate and concentrated under vacuum. The residue was purified by flash column chromatography on silica gel (PE/EtOAc=10/1) to give 3-bromo-N [symbol] (tert-butyldimethylsilyl)benzenesulfonimidamide (2 g, 50.4%) as a white solid. LCMS (ESI) calcd. for C₁₂H₂₂BrN₂OSSi [M+H]⁺ m/z 351.04, found 350.95.

Step 3: tert-butyl (3-bromo-N-(tert-butyldimethylsilyl)phenylsulfonimidoyl)(tert-butoxycarbonyl)carbamate: A solution of 3-bromo-N [symbol] tert-butyldimethylsilyl)benzenesulfonimidamide (2 g, 5.73 mmol), DMAP (839 mg, 6.87 mmol) and di-tert-butyl dicarbonate (4.99 g, 22.92 mmol) in THE (20 mL) stirred at room temperature for 3 hours. Then the mixture was diluted with water (40 mL) and extracted with DCM (40 mL×3). The combined organic phases were washed with brine, dried over sodium sulfate, and concentrated under vacuum. The residue was purified by flash column chromatography on silica gel (PE/EtOAc=20/1) to give tert-butyl (3-bromo-N-(tert-butyldimethylsilyl)phenylsulfonimidoyl)(tert-butoxycarbonyl)carbamate(1.29 g, 41.0%) as a white solid. LCMS (ESI) calcd. for C₂₂H₃₈BrN₂O₅SSi [M+H]⁺ m/z 551.15, found 551.15.

Step 4: tert-butyl (tert-butoxycarbonyl)(3-(3-(4,4-difluoroazepan-1-yl)-5-methyl-6-(trifluoromethyl)pyridazine-4-carboxamido)phenylsulfonimidoyl)carbamate: A mixture of 3-(4,4-difluoroazepan-1-yl)-5-methyl-6-(trifluoromethyl)pyridazine-4-carboxamide (150 mg, 0.44 mmol), tert-butyl (3-bromo-N-(tert-butyldimethylsilyl)phenylsulfonimidoyl)(tert-butoxycarbonyl)carbamate (362 mg, 0.66 mmol), Cs₂CO₃ (287 mg, 0.88 mmol) and Xantphos-Pd-G2 (35 mg, 0.044 mmol) in 1,4-dioxane (10 mL) was heated at 100° C. for 16 hours under an atmosphere of N₂. After the reaction was completed, the mixture was concentrated under vacuum. The residue was directly purified by flash column chromatography on silica gel (PE/EtOAc=1/2) to give tert-butyl (tert-butoxycarbonyl)(3-(3-(4,4-difluoroazepan-1-yl)-5-methyl-6-(trifluoromethyl)pyridazine-4-carboxamido)phenylsulfonimidoyl)carbamate (75 mg, 24.6%) as a yellow solid. LCMS (ESI) calcd. for C₂₉H₃₈F₅N₆O₆S [M+H]⁺ m/z 693.25, found 693.20.

Step 5: 3-(4,4-difluoroazepan-1-yl)-5-methyl-N-(3-sulfamidimidoylphenyl)-6-(trifluoromethyl)pyridazine-4-carboxamide: A solution of tert-butyl (tert-butoxycarbonyl)(3-(3-(4,4-difluoroazepan-1-yl)-5-methyl-6-(trifluoromethyl)pyridazine-4-carboxamido)phenylsulfonimidoyl)carbamate (75 mg, 0.11 mmol) in 1,4-dioxane (1 mL) was added 4M HCl dioxane (1 mL) at room temperature. The solution was stirred at room temperature for 2 hours then concentrated under vacuum. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 35% to 95% MeCN/H₂O containing 0.1% NH₃) to obtain 3-(4,4-difluoroazepan-1-yl)-5-methyl-N-(3-sulfamidimidoylphenyl)-6-(trifluoromethyl)pyridazine-4-carboxamide (18.3 mg, 33.7%) as a white solid. ¹H NMR (400 MHz, DMSO-d₆, ppm) δ 11.04 (s, 1H), 8.27-8.23 (m, 1H), 7.76-7.71 (m, 2H), 7.51 (s, 1H), 3.85 (s, 2H), 3.71 (s, 2H), 2.41-2.34 (m, 2H), 2.32 (s, 3H), 2.11-2.03 (m, 2H), 1.91-1.86 (m, 2H). LCMS (ESI) calcd. for C₁₉H₂₂F₅N₆O₂S [M+H]⁺ m/z 493.15, found 493.05.

### Example 116

### (R)-2-(4,4-difluoroazepan-1-yl)-4-methyl-N-(3-(S-methylsulfonimidoyl)phenyl)-5-(2-oxa-6-azaspiro[3.3]heptan-6-1)nicotinamide

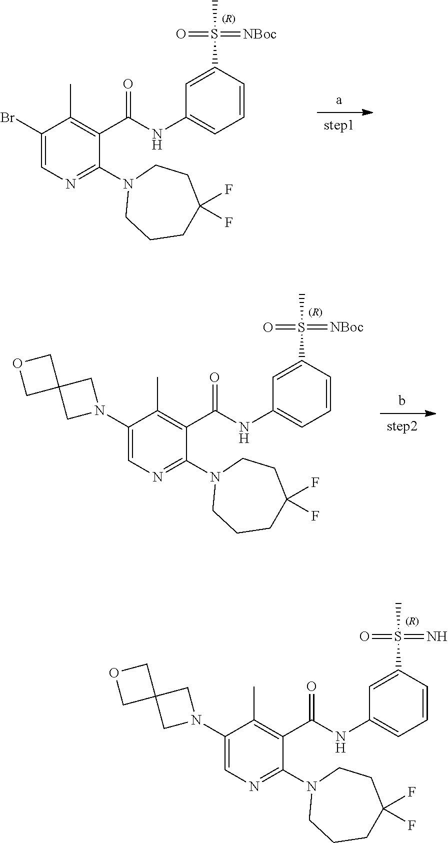

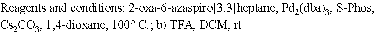

Step 1: tert-butyl (R)-((3-(2-(4,4-difluoroazepan-1-yl)-4-methyl-5-(2-oxa-6-azaspiro[3.3]heptan-6-yl)nicotinamido)phenyl)(methyl)(oxo)-λ⁶-sulfaneylidene)carbamate: A mixture of tert-butyl (R)-((3-(5-bromo-2-(4,4-difluoroazepan-1-yl)-4-methylnicotinamido)phenyl)(methyl)(oxo)-λ⁶-sulfaneylidene)carbamate(80 mg, 0.133 mmol), 2-oxa-6-azaspiro[3.3]heptane (20 mg, 0.200 mmol), Cs₂CO₃ (130 mg, 0.400 mmol) and Pd₂(dba)₃ (12 mg, 0.013 mmol), S-Phos (5 mg, 0.013 mmol) in 1,4-dioxane (10 mL) was heated at 100° C. for 16 hours under an atmosphere of N₂. After the reaction was completed, the mixture was concentrated under vacuum. The residue was purified by flash column chromatography on silica gel (PE/EtAOc=1/1) to give tert-butyl (R)-((3-(2-(4,4-difluoroazepan-1-yl)-4-methyl-5-(2-oxa-6-azaspiro[3.3]heptan-6-yl)nicotinamido)phenyl)(methyl)(oxo)-λ⁶-sulfaneylidene)carbamate (60 mg, 73.17%) as a yellow oil. LCMS (ESI) calcd. for C₃₀H₄₀F₂N₅O₅S [M+H]⁺ m/z 620.27, found 620.25.

Step 2: (R)-2-(4,4-difluoroazepan-1-yl)-4-methyl-N-(3-(S-methylsulfonimidoyl)phenyl)-5-(2-oxa-6-azaspiro[3.3]heptan-6-yl)nicotinamide: A solution of tert-butyl (R)-((3-(2-(4,4-difluoroazepan-1-yl)-4-methyl-5-(2-oxa-6-azaspiro[3.3]heptan-6-yl)nicotinamido)phenyl)(methyl)(oxo)-λ⁶-sulfaneylidene)carbamate (60 mg, 0.097 mmol) in DCM (3 mL) was added TFA (0.3 mL) at room temperature. The solution was stirred at room temperature for 2 hours then concentrated under vacuum. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 55% to 90% MeCN/H₂O containing 0.1% formic acid) to obtain (R)-2-(4,4-difluoroazepan-1-yl)-4-methyl-N-(3-(S-methylsulfonimidoyl)phenyl)-5-(2-oxa-6-azaspiro[3.3]heptan-6-yl)nicotinamide (28 mg, 56.80%) as a white solid. ¹H NMR (400 MHz, DMSO-d₆) δ 10.72 (s, 1H), 8.36 (s, 1H), 7.89 (d, J=7.8 Hz, 1H), 7.68-7.62 (m, 1H), 7.60-7.52 (m, 2H), 4.71 (s, 4H), 4.21 (s, 1H), 3.99 (s, 4H), 3.34 (s, 4H), 3.04 (s, 3H), 2.24-2.12 (m, 2H), 2.06 (s, 3H), 2.03-1.92 (m, 2H), 1.76-1.66 (m, 2H). LCMS (ESI) calcd. for C₂₅H₃₂F₂N₅O₃S [M+H]⁺ m/z 520.22, found 520.35.

### Example 117 and 118

### (S)-3-(4,4-difluoroazepan-1-yl)-5-methyl-N-(3-(S-methylsulfonimidoyl)phenyl)-6-phenylpyridazine-4-carboxamide and (R)-3-(4,4-difluoroazepan-1-yl)-5-methyl-N-(3-(S-methylsulfonimidoyl)phenyl)-6-phenylpyridazine-4-carboxamide

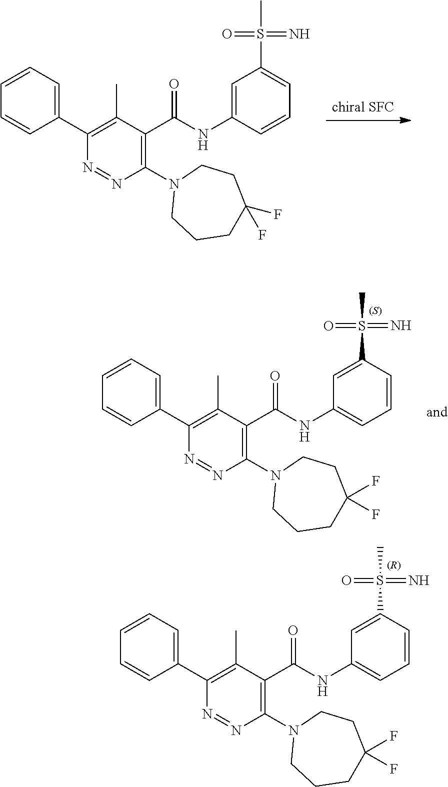

3-(4,4-difluoroazepan-1-yl)-5-methyl-N-(3-(S-methylsulfonimidoyl)phenyl)-6-phenylpyridazine-4-carboxamide (40 mg) was purified by Chiral SFC (chiralpak-AS-H, 150*21.2 mm, eluting with 25% CO₀₂-MeOH containing 0.1% formic acid). The first elution was lyophilized to afford (S)-3-(4,4-difluoroazepan-1-yl)-5-methyl-N-(3-(S-methylsulfonimidoyl)phenyl)-6-phenylpyridazine-4-carboxamide (13.7 mg, 34%) as a white solid. ¹H NMR (400 MHz, DMSO-d₆) δ 11.10 (s, 1H), 8.33-8.31 (m, 1H), 7.89 (d, J=8 Hz, 1H), 7.70 (d, J=8 Hz, 1H), 7.63-7.59 (m, 1H), 7.57-7.46 (m, 5H), 4.26 (s, 1H), 3.79-3.77 (m, 2H), 3.67-3.64 (m, 2H), 3.07 (s, 3H), 2.35-2.33 (m, 2H), 2.19 (s, 3H), 2.06-2.04 (m, 2H), 1.88-1.86 (m, 2H). LCMS (ESI) calcd. for C₂₅H₂₈F₂N₅O₂S [M+H]⁺ m/z 500.19, found 500.15. and the second elution was lyophilized to afford (R)-3-(4,4-difluoroazepan-1-yl)-5-methyl-N-(3-(S-methylsulfonimidoyl)phenyl)-6-phenylpyridazine-4-carboxamide (13.3 mg, 33%) as a white solid. ¹H NMR (400 MHz, DMSO-d₆) δ 11.10 (s, 1H), 8.33-8.31 (m, 1H), 7.88 (d, J=8.8 Hz, 1H), 7.70 (d, J=8 Hz, 1H), 7.63-7.59 (m, 1H), 7.57-7.53 (m, 2H), 7.51-7.48 (m, 2H), 7.48-7.46 (m, 1H), 4.25 (s, 1H), 3.79-3.77 (m, 2H), 3.67-3.64 (m, 2H), 3.07 (s, 3H), 2.35-2.33 (m, 2H), 2.19 (s, 3H), 2.06-2.04 (m, 2H), 1.88-1.86 (m, 2H). LCMS (ESI) calcd. for C₂₅H₂₈F₂N₅O₂S [M+H]⁺ m/z 500.19, found 500.15.

### Example 119

### 3-(4,4-difluoroazepan-1-yl)-5-methyl-N-(2-sulfamidimidoylpyridin-4-yl)-6-(trifluoromethyl)pyridazine-4-carboxamide

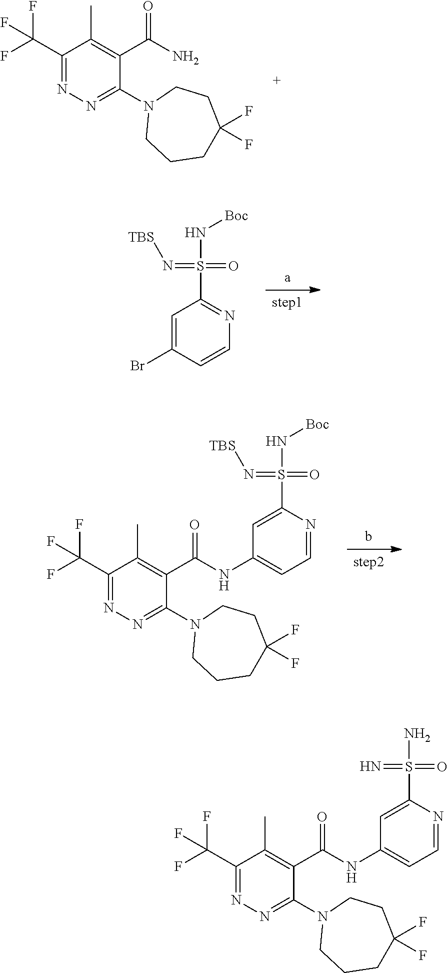

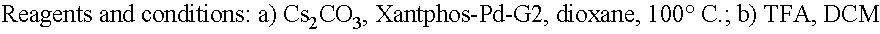

Step 1: A solution of 3-(4,4-difluoroazepan-1-yl)-5-methyl-6-(trifluoromethyl)pyridazine-4-carboxamide (250 mg, 0.74 mmol) in dioxane (12 mL) was added tert-butyl (4-bromo-N-(tert-butyldimethylsilyl)pyridine-2-sulfonimidoyl)carbamate (367 mg, 0.81 mmol), cesium carbonate (626 mg, 1.92 mmol) and XantPhos-Pd-G2 (65.7 mg, 0.074 mmol) at room temperature. The reaction mixture was heated at 100° C. for 16 hours under nitrogen. After the reaction was completed, the mixture was diluted with water (30 mL) and extracted with DCM (30 mL×3). The combined organic phases were washed with brine, dried over sodium sulfate, concentrated under vacuum. The residue was purified by flash column chromatography on silica gel (PE/EtOAc=1/1) to give tert-butyl (N-(tert-butyldimethylsilyl)-4-(3-(4,4-difluoroazepan-1-yl)-5-methyl-6-(trifluoromethyl)pyridazine-4-carboxamido)pyridine-2-sulfonimidoyl)carbamate (180 mg, 34.4%) as a yellow oil. LCMS (ESI) calcd. for C₂₉H₄₃F₅N₇O₄SSi [M+H]⁺ m/z 708.28, found 708.25.

Step 2: To a solution of tert-butyl (N-(tert-butyldimethylsilyl)-4-(3-(4,4-difluoroazepan-1-yl)-5-methyl-6-(trifluoromethyl)pyridazine-4-carboxamido)pyridine-2-sulfonimidoyl)carbamate (180 mg, 0.25 mmol) in DCM (7.5 mL) was added TFA (1.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (30 mL×3). The combined organic phases were washed with brine, dried over sodium sulfate, concentrated under vacuum. The residue was purified by prep-HPLC (Gemini 5 um C₁₈ column, 150*21.2 mm, eluting with 30% to 85% MeCN/H₂O containing 0.05% NH₃) to afford 3-(4,4-difluoroazepan-1-yl)-5-methyl-N-(2-sulfamidimidoylpyridin-4-yl)-6-(trifluoromethyl)pyridazine-4-carboxamide (53.5 mg, 42.7%) as a white solid. ¹H NMR (400 MHz, DMSO-d₆) δ 11.45 (s, 1H), 8.53 (s, 1H), 8.25 (s, 1H), 7.78-7.63 (m, 1H), 5.8-6.9 (bs, 2H), 3.84 (s, 2H), 3.68 (s, 2H), 2.33 (s, 2H), 2.30 (s, 3H), 2.06 (d, J=13.6 Hz, 2H), 1.87 (d, J=5.3 Hz, 2H). LCMS (ESI) calcd. for C₁₈H₂₁F₅N₇O₂S [M+H]⁺ m/z 494.14, found 494.10.

### Intermediate 1

### di-tert-butyl ((3-bromophenyl)(methyl)-λ⁶-sulfanediylidene)dicarbamate

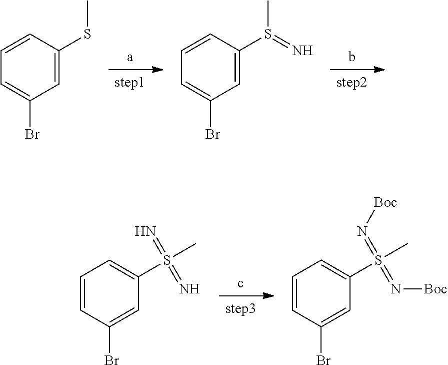

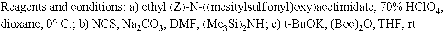

Step 1: (3-bromophenyl)(methyl)-λ⁴-sulfanimine: To a solution of ethyl (Z)-N-((mesitylsulfonyl)oxy)acetimidate (4.4 g, 15.4 mmol) in dioxane (15 mL) was added HClO₄ (70%, 15 mL) dropwise at 0° C. The mixture was stirred 0° C. for 15 minutes. The mixture was quenched with cold water (30 mL) and extracted with DCM (15 mL×3). The combined organic phases were washed with brine, dried over sodium sulfate. Then a solution of (3-bromophenyl)(methyl)sulfane (2 g, 9.85 mmol) in DCM (15 mL) was added dropwise to the mixture at 0° C. The mixture was stirred at room temperature for 2 h. After the reaction was completed, half of the DCM was evaporated, and diethyl ether (50 mL) was added to the mixture. After storing at 0° C. overnight, the crystal precipitation was collected and dried under vacuum to provide (3-bromophenyl)(methyl)-λ⁴-sulfanimine (1.5 g, 70%) was crystallized upon storage at 0° C. overnight. LCMS (ESI) calcd. for C₇H₉BrNS [M+H]⁺ m/z 219.96, found 219.90.

Step 2: (3-bromophenyl)(methyl)-λ⁶-sulfanediimine: To a solution of (3-bromophenyl)(methyl)-λ⁴-sulfanimine (1.5 g, 6.88 mmol) in DMF (15 mL) was added NCS (1.1 g, 0.008 mol) and Na₂CO₃ (4.3 g, 0.041 mol) at 0° C. under N₂. The mixture was stirred at 0° C. for 15 minutes. Then (Me₃Si)₂NH (3.3 g, 0.021 mol) was added to the mixture at 0° C. The mixture was stirred at room temperature for 16 hours. After the reaction was completed, the mixture was cooled to room temperature. The resulting solution was diluted with water (30 mL) and extracted with DCM (30 mL×3). The combined organic phases were washed with brine, dried over Na₂SO₄, concentrated under vacuum. The residue was purified by prep-HPLC (Gemini 5 um C₁₈ column, 150*21.2 mm, eluting with 5% to 30% MeCN/H₂O containing 0.05% NH₃) to provide (3-bromophenyl)(methyl)-λ⁶-sulfanediimine (200 mg, 12.48% yield) as a white solid. LCMS (ESI) calcd. for C₇H₁₀BrN₂S [M+H]⁺ m/z 232.98, found 232.85.

Step 3: di-tert-butyl ((3-bromophenyl)(methyl)-λ⁶-sulfanediylidene)dicarbamate: A (3-bromophenyl)(methyl)-λ⁶-sulfanediimine (200 mg, 0.862 mmol), t-BuOK (241 mg, 2.155 mmol) and Boc₂O (563 mg, 2.586 mmol) in THE (10 mL) was stirred at room temperature for 16 h. After the reaction was completed, the mixture was cooled to room temperature. The resulting solution was diluted with water (30 mL) and extracted with EtOAc (30 mL×3). The combined organic phases were washed with brine, dried over Na₂SO₄, concentrated under vacuum. The residue was purified by flash column chromatography on silica gel (PE/EtAOc=3/1) to give di-tert-butyl ((3-bromophenyl)(methyl)-λ⁶-sulfanediylidene)dicarbamate (80 mg, 21.51%) as a yellow solid. LCMS (ESI) calcd. for C₁₇H₂₆BrN₂O₄S [M+H]⁺ m/z 433.08, found 433.00.

### Example 120

### 3-(4,4-difluoroazepan-1-yl)-N-{3-[diimino(methyl)-λ⁶-sulfanyl]phenyl}-5-methyl-6-(trifluoromethyl)pyridazine-4-carboxamide

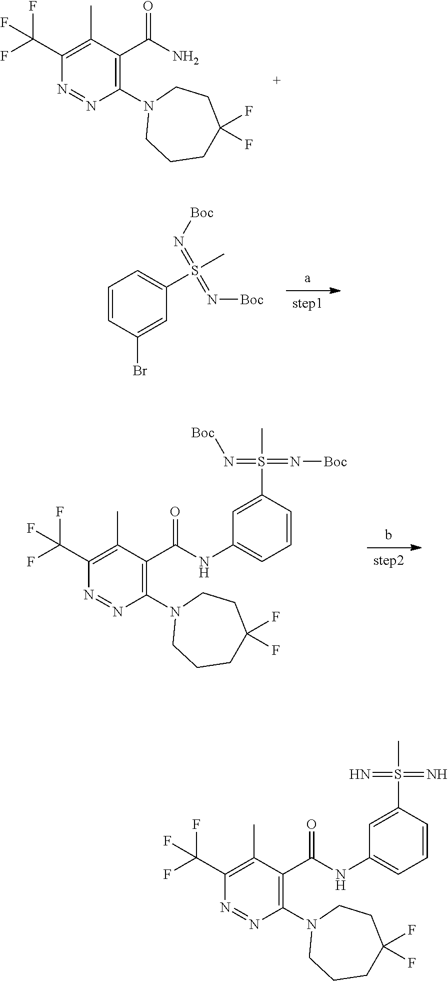 [SMILES: Cc1c(C(F)(F)F)nnc(N2CCCC(F)(F)CC2)c1C(N)=O ; CC(C)(C)OC(=O)N=S(C)(=NC(=O)OC(C)(C)C)c1cccc(Br)c1 ; Cc1c(C(F)(F)F)nnc(N2CCCC(F)(F)CC2)c1C(=O)Nc1cccc(S(C)(=NC(=O)OC(C)(C)C)=NC(=O)OC(C)(C)C)c1 ; Cc1c(C(F)(F)F)nnc(N2CCCC(F)(F)CC2)c1C(=O)Nc1cccc(S(C)(=N)=N)c1]

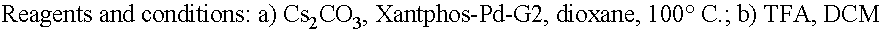

Step 1: di-tert-butyl ((3-(3-(4,4-difluoroazepan-1-yl)-5-methyl-6-(trifluoromethyl)pyridazine-4-carboxamido)phenyl)(methyl)-λ⁶-sulfanediylidene)dicarbamate: A mixture of 3-(4,4-difluoroazepan-1-yl)-5-methyl-6-(trifluoromethyl)pyridazine-4-carboxamide (80 mg, 0.237 mmol), di-tert-butyl ((3-bromophenyl)(methyl)-λ⁶-sulfanediylidene)dicarbamate (80 mg, 0.185 mmol), Cs₂CO₃ (231 mg, 0.711 mmol) and Xantphos-Pd-G2 (21 mg, 0.024 mmol) in dioxane (10 mL) was heated at 100° C. for 16 h under an atmosphere of N₂. After the reaction was completed, the mixture was cooled to room temperature. The resulting solution was diluted with water (30 mL) and extracted with EtOAc (30 mL×3). The combined organic phases were washed with brine, dried over Na₂SO₄, concentrated under vacuum. The residue was purified by flash column chromatography on silica gel (PE/EtAOc=1/1) to give di-tert-butyl ((3-(3-(4,4-difluoroazepan-1-yl)-5-methyl-6-(trifluoromethyl)pyridazine-4-carboxamido)phenyl)(methyl)-λ⁶-sulfanediylidene)dicarbamate (30 mg, 18.41%) as a yellow oil. LCMS (ESI) calcd. for C₃₀H₄₀F₅N₆O₅S [M+H]⁺ m/z 691.27, found 691.15.

Step 2: 3-(4,4-difluoroazepan-1-yl)-N-{3-[diimino(methyl)-λ⁶-sulfanyl]phenyl}-5-methyl-6-(trifluoromethyl)pyridazine-4-carboxamide: A solution of di-tert-butyl ((3-(3-(4,4-difluoroazepan-1-yl)-5-methyl-6-(trifluoromethyl)pyridazine-4-carboxamido)phenyl)(methyl)-λ⁶-sulfanediylidene)dicarbamate (30 mg, 0.043 mmol) in DCM (2 mL) was added TFA (0.2 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 45% to 95% MeCN/H₂O containing 0.1% formic acid) to provide 3-(4,4-difluoroazepan-1-yl)-N-{3-[diimino(methyl)-λ⁶-sulfanyl]phenyl}-5-methyl-6-(trifluoromethyl)pyridazine-4-carboxamide (3.1 mg, 14.6%) as a white solid. ¹H NMR (400 MHz, DMSO-d₆) δ 11.10 (s, 1H), 8.35 (s, 1H), 7.86 (dd, J=17.8, 7.9 Hz, 2H), 7.59 (td, J=7.9, 2.9 Hz, 1H), 3.85 (s, 2H), 3.69 (s, 2H), 3.02 (d, J=2.9 Hz, 3H), 2.33 (s, 5H), 2.14-2.00 (m, 2H), 1.88 (s, 2H). LCMS (ESI) calcd. for C₂₀H₂₄F₅N₆OS [M+H]⁺ m/z 491.16, found 491.05.

### Example 121

### 2-(4,4-difluoroazepan-1-yl)-6-methyl-N-(3-sulfamidimidoylphenyl)-5-(trifluoromethyl)nicotinamide

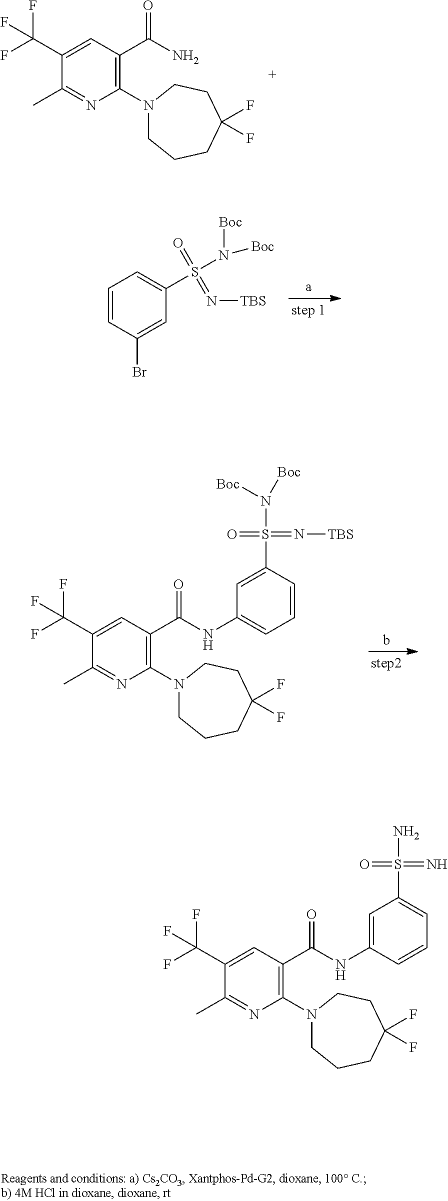

Step 1: tert-butyl (tert-butoxycarbonyl)(3-(2-(4,4-difluoroazepan-1-yl)-6-methyl-5-(trifluoromethyl)nicotinamido)phenylsulfonimidoyl)carbamate: A solution of 2-(4,4-difluoroazepan-1-yl)-6-methyl-5-(trifluoromethyl)nicotinamide (200 mg, 0.593 mmol) in dioxane (10 mL) was tert-butyl (3-bromo-N-(tert-butyldimethylsilyl)phenylsulfonimidoyl)(tert-butoxycarbonyl)carbamate (975 mg, 1.779 mmol), cesium carbonate (502 mg, 1.542 mmol) and XantPhos-Pd-G2 (106 mg, 0.119 mmol) at room temperature. The reaction mixture was heated at 100° C. for 16 hours under nitrogen. After the reaction was completed, the mixture was diluted with water (20 mL) and extracted with DCM (20 mL×3). The combined organic phases were washed with brine, dried over sodium sulfate, concentrated under vacuum. The residue was purified by flash column chromatography on silica gel (PE/EtOAc=1/1) to give tert-butyl (tert-butoxycarbonyl)(3-(2-(4,4-difluoroazepan-1-yl)-6-methyl-5-(trifluoromethyl)nicotinamido)phenylsulfonimidoyl)carbamate (100 mg, 24.39%) as a white oil. LCMS (ESI) calcd. for C₃₀H₃₉F₅N₅O₆S [M+H]⁺ m/z 692.25, found 692.20.

Step 2: 2-(4,4-difluoroazepan-1-yl)-6-methyl-N-(3-sulfamidimidoylphenyl)-5-(trifluoromethyl)nicotinamide: To a solution of tert-butyl (tert-butoxycarbonyl)(3-(2-(4,4-difluoroazepan-1-yl)-6-methyl-5-(trifluoromethyl)nicotinamido)phenylsulfonimidoyl)carbamate (50 mg, 0.072 mmol) in 1,4-dioxane (5 mL) was added 4M HCl-dioxane (5 mL) at room temperature. The reaction mixture was stirred at room temperature for 1 hour. After the reaction was completed, the mixture was diluted with water (20 mL) and extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 35% to 95% MeCN/H₂O containing 0.1% formic acid) to afford 2-(4,4-difluoroazepan-1-yl)-6-methyl-N-(3-sulfamidimidoylphenyl)-5-(trifluoromethyl)nicotinamide (16.23 mg, 54.10%) as a white solid. ¹H NMR (400 MHz, DMSO-d₆) δ 10.72 (s, 1H), 8.28-8.26 (m, 1H), 7.90 (s, 1H), 7.82 (d, J=8.4 Hz, 1H), 7.64 (d, J=8 Hz, 1H), 7.50-7.46 (m, 1H), 6.5-5.5 (bs, 2H), 3.72-3.70 (m, 2H), 3.49-3.46 (m, 2H), 2.49 (s, 3H), 2.33-2.30 (m, 2H), 1.98-1.92 (m, 2H), 1.88-1.87 (m, 2H). LCMS (ESI) calcd. for C₂₀H₂₃F₅N₅O₂S [M+H]⁺ m/z 492.15, found 492.10.

### Example 122

### (R)-2-(4,4-difluoroazepan-1-yl)-4-methyl-N-(3-(S-methylsulfonimidoyl)phenyl)-5-phenylnicotinamide

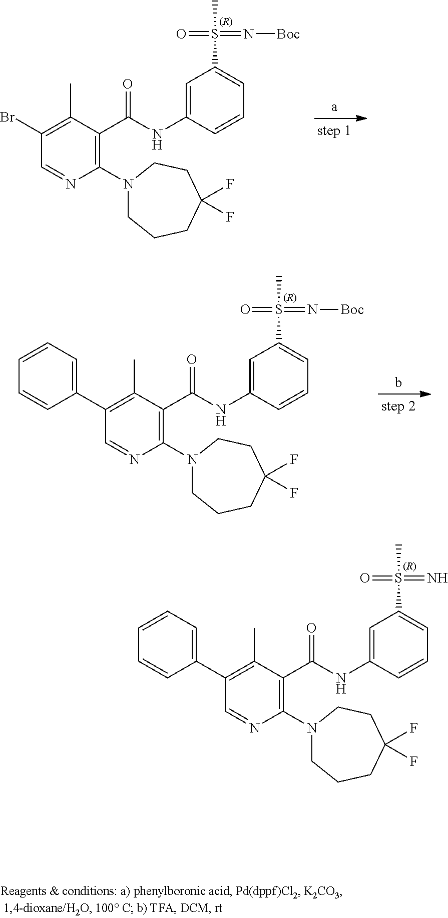

Step 1: tert-butyl (R)-((3-(2-(4,4-difluoroazepan-1-yl)-4-methyl-5-phenylnicotinamido)phenyl)(methyl)(oxo)-λ⁶-sulfaneylidene)carbamate: A mixture of tert-butyl (R)-((3-(5-bromo-2-(4,4-difluoroazepan-1-yl)-4-methylnicotinamido)phenyl)(methyl)(oxo)-λ⁶-sulfaneylidene)carbamate (100 mg, 0.17 mmol), phenylboronic acid (41 mg, 0.34 mmol), K₂CO₃ (47 mg, 0.34 mmol) and Pd(dppf)Cl₂ (7 mg, 0.01 mmol) in 1,4-dioxane/H₂O (4/1, 10 mL) was heated at 100° C. for 16 hours under an atmosphere of N₂. After the reaction was completed, the mixture was concentrated under vacuum. The residue was directly purified by flash column chromatography on silica gel (PE/EtAOc=3/1) to give tert-butyl (R)-((3-(2-(4,4-difluoroazepan-1-yl)-4-methyl-5-phenylnicotinamido)phenyl)(methyl)(oxo)-λ⁶-sulfaneylidene)carbamate (50 mg, 83.6%) as a yellow solid. LCMS (ESI) calcd. for C₃₁H₃₇F₂N₄O₄S [M+H]⁺ m/z 599.25, found 599.20.

Step 2: (R)-2-(4,4-difluoroazepan-1-yl)-4-methyl-N-(3-(S-methylsulfonimidoyl)phenyl)-5-phenylnicotinamide: A solution of tert-butyl (R)-((3-(2-(4,4-difluoroazepan-1-yl)-4-methyl-5-phenylnicotinamido)phenyl)(methyl)(oxo)-λ⁶-sulfaneylidene)carbamate(50 mg, 0.08 mmol) in DCM (3 mL) was added TFA (0.3 mL) at room temperature. The reaction mixture was stirred at room temperature for 2 hours. After the reaction was completed, the mixture was concentrated, and the residue was adjusted to pH=8-9 with saturated aqueous NaHCO₃. The aqueous solution was extracted with DCM (30 mL×3). The combined organic phases were washed with brine, dried over sodium sulfate, concentrated under vacuum. The residue was purified by prep-HPLC (Gemini 5 um C₁₈ column, 150*21.2 mm, eluting with 40% to 95% MeCN/H₂O containing 0.1% NH₃) to provide (R)-2-(4,4-difluoroazepan-1-yl)-4-methyl-N-(3-(S-methylsulfonimidoyl)phenyl)-5-phenylnicotinamide (16.25 mg, 40.7%) as a white solid. ¹H NMR (400 MHz, DMSO-d₆) δ 10.88 (s, 1H), 8.38 (s, 1H), 8.08 (s, 1H), 7.90 (d, J=8.1 Hz, 1H), 7.66 (d, J=8.2 Hz, 1H), 7.58 (t, J=7.9 Hz, 1H), 7.47 (t, J=7.3 Hz, 2H), 7.42-7.31 (m, 3H), 4.22 (s, 1H), 3.71-3.54 (m, 4H), 3.06 (s, 3H), 2.36-2.21 (m, 2H), 2.15 (s, 3H), 2.09-1.94 (m, 2H), 1.87-1.73 (m, 2H). LCMS (ESI) calcd. for C₂₆H₂₉F₂N₄O₂S [M+H]⁺ m/z 499.20, found 499.15.

### Example 123

### (R)-2-(4,4-difluoroazepan-1-yl)-5-(3-hydroxyprop-1-yn-1-yl)-4-methyl-N-(3-(S-methylsulfonimidoyl)phenyl)nicotinamide

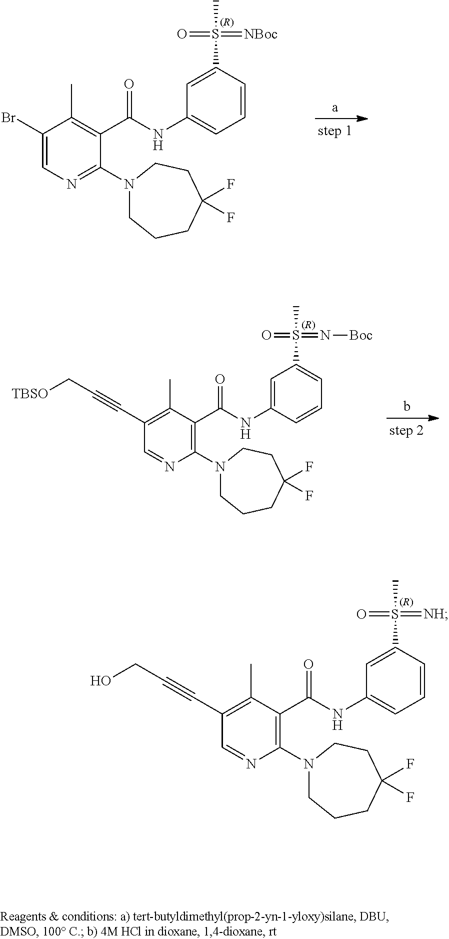

Step 1: tert-butyl (R)-((3-(5-(3-((tert-butyldimethylsilyl)oxy)prop-1-yn-1-yl)-2-(4,4-difluoroazepan-1-yl)-4-methylnicotinamido)phenyl)(methyl)(oxo)-λ⁶-sulfaneylidene)carbamate: A mixture of tert-butyl (R)-((3-(5-bromo-2-(4,4-difluoroazepan-1-yl)-4-methylnicotinamido)phenyl)(methyl)(oxo)-λ⁶-sulfaneylidene)carbamate (80 mg, 0.133 mmol), tert-butyldimethyl(prop-2-yn-1-yloxy)silane (45 mg, 0.267 mmol), DBU (41 mg, 0.267 mmol) and Pd(PPh₃)₄ (15 mg, 0.013 mmol) in DMSO (5 mL) was heated at 100° C. for 16 h under an atmosphere of N₂. After the reaction was completed, the mixture was cooled to room temperature. The resulting solution was diluted with water (30 mL) and extracted with EtOAc (30 mL×3). The combined organic phases were washed with brine, dried over Na₂SO₄, concentrated under vacuum. The residue was purified by flash column chromatography on silica gel (PE/EtAOc=1/1) to give tert-butyl (R)-((3-(5-(3-((tert-butyldimethylsilyl)oxy)prop-1-yn-1-yl)-2-(4,4-difluoroazepan-1-yl)-4-methylnicotinamido)phenyl)(methyl)(oxo)-λ⁶-sulfaneylidene)carbamate (50 mg, 54.35%) as a yellow oil. LCMS (ESI) calcd. for C₃₄H₄₉F₂N₄O₅SSi [M+H]⁺ m/z 691.31, found 691.30.

Step 2: (R)-2-(4,4-difluoroazepan-1-yl)-5-(3-hydroxyprop-1-yn-1-yl)-4-methyl-N-(3-(S-methylsulfonimidoyl)phenyl)nicotinamide: A solution of tert-butyl (R)-((3-(5-(3-((tert-butyldimethylsilyl)oxy)prop-1-yn-1-yl)-2-(4,4-difluoroazepan-1-yl)-4-methylnicotinamido)phenyl)(methyl)(oxo)-λ⁶-sulfaneylidene)carbamate (50 mg, 0.072 mmol) in 1,4-dioxane (3 mL) was added HCl-1,4-dioxane (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 45% to 95% MeCN/H₂O containing 0.1% formic acid) to provide (R)-2-(4,4-difluoroazepan-1-yl)-5-(3-hydroxyprop-1-yn-1-yl)-4-methyl-N-(3-(S-methylsulfonimidoyl)phenyl)nicotinamide (15.2 mg, 35.59%) as a white solid. ¹H NMR (400 MHz, DMSO-d₆) δ 10.85 (s, 1H), 8.34 (t, J=1.8 Hz, 1H), 8.22 (s, 1H), 7.89 (d, J=8.9 Hz, 1H), 7.67 (d, J=8.2 Hz, 1H), 7.59 (t, J=7.9 Hz, 1H), 5.33 (t, J=5.9 Hz, 1H), 4.33 (d, J=5.9 Hz, 2H), 4.23 (s, 1H), 3.65 (dd, J=6.4, 3.9 Hz, 2H), 3.56 (t, J=6.1 Hz, 2H), 3.06 (d, J=0.7 Hz, 3H), 2.35-2.20 (m, 5H), 2.04-1.94 (m, 2H), 1.84-1.76 (m, 2H). LCMS (ESI) calcd. for C₂₃H₂₇F₂N₄O₃S [M+H]⁺ m/z 477.18, found 477.15.

### Example 124

### (R)-2-(4,4-difluoroazepan-1-yl)-5-(3-(dimethylamino)prop-1-yn-1-yl)-4-methyl-N-(3-(S-methylsulfonimidoyl)phenyl)nicotinamide

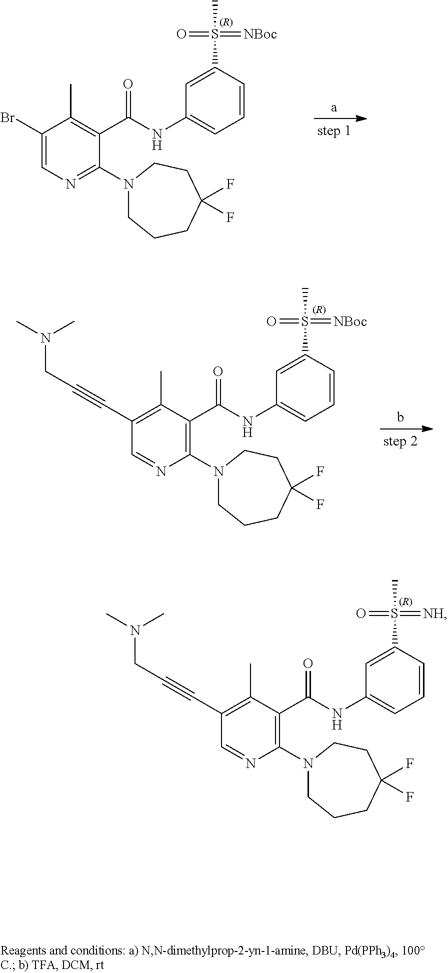

Step 1: tert-butyl (R)-((3-(2-(4,4-difluoroazepan-1-yl)-5-(3-(dimethylamino)prop-1-yn-1-yl)-4-methylnicotinamido)phenyl)(methyl)(oxo)-λ⁶-sulfaneylidene)carbamate: To a solution of tert-butyl (R)-((3-(5-bromo-2-(4,4-difluoroazepan-1-yl)-4-methylnicotinamido)phenyl)(methyl)(oxo)-λ⁶-sulfaneylidene)carbamate (100 mg, 0.17 mmol), N,N-dimethylprop-2-yn-1-amine (16 mg, 0.19 mmol) and DBU (57 mg, 0.37 mmol) in DMSO (2 mL) was added Pd(PPh₃)₄ (10 mg, 0.0085 mmol). Then the mixture was heated at 100° C. for 12 hours under N₂. LCMS showed the reaction was completed. The mixture was diluted with water and extracted with EtOAc (3×5 mL). The combined organic phases were washed with brine, dried with Na₂SO₄, and concentrated under vacuum. The residue was purified by silica gel column chromatography (eluting with MeOH/DCM, 0 to 10%) to give tert-butyl (R)-((3-(2-(4,4-difluoroazepan-1-yl)-5-(3-(dimethylamino)prop-1-yn-1-yl)-4-methylnicotinamido)phenyl)(methyl)(oxo)-λ⁶-sulfaneylidene)carbamate(70 mg, 68.29% yield) as a yellow oil. LCMS (ESI) calcd. for C₃₀H₄₀F₂N₅O₄S [M+H]⁺ m/z 604.28, found 604.25.

Step 2: (R)-2-(4,4-difluoroazepan-1-yl)-5-(3-(dimethylamino)prop-1-yn-1-yl)-4-methyl-N-(3-(S-methylsulfonimidoyl)phenyl)nicotinamide: A solution of tert-butyl (R)-((3-(2-(4,4-difluoroazepan-1-yl)-5-(3-(dimethylamino)prop-1-yn-1-yl)-4-methylnicotinamido)phenyl)(methyl)(oxo)-λ⁶-sulfaneylidene)carbamate (70 mg, 0.12 mmol) in DCM (2 mL) was added TFA (0.2 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (10 mL×3). The combined organic phases were washed with brine, dried over sodium sulfate, concentrated under vacuum. The residue was purified by prep-HPLC (Gemini 5 um C₁₈ column, 150*21.2 mm, eluting with 40% to 95% MeCN/H₂O containing 0.05% NH₃) to afford (R)-2-(4,4-difluoroazepan-1-yl)-5-(3-(dimethylamino)prop-1-yn-1-yl)-4-methyl-N-(3-(S-methylsulfonimidoyl)phenyl)nicotinamide (20.17 mg, 33.42%) as a white solid. LCMS (ESI) calcd. for C₂₅H₃₂F₂N₅O₂S [M+H]⁺ m/z 504.22, found 504.25. ¹H NMR (400 MHz, DMSO-d₆) δ 10.84 (s, 1H), 8.28 (d, J=33.4 Hz, 2H), 7.89 (d, J=7.4 Hz, 1H), 7.69-7.57 (m, 2H), 4.23 (s, 1H), 3.68-3.54 (m, 4H), 3.49 (s, 2H), 3.06 (s, 3H), 2.33 (s, 3H), 2.24 (s, 6H), 1.99 (d, J=7.8 Hz, 2H), 1.81 (d, J=4.9 Hz, 2H), 1.23 (s, 2H).

### Example 125

### (R)-5-cyano-2-(4,4-difluoroazepan-1-yl)-4-methyl-N-(3-(S-methylsulfonimidoyl)phenyl)nicotinamide

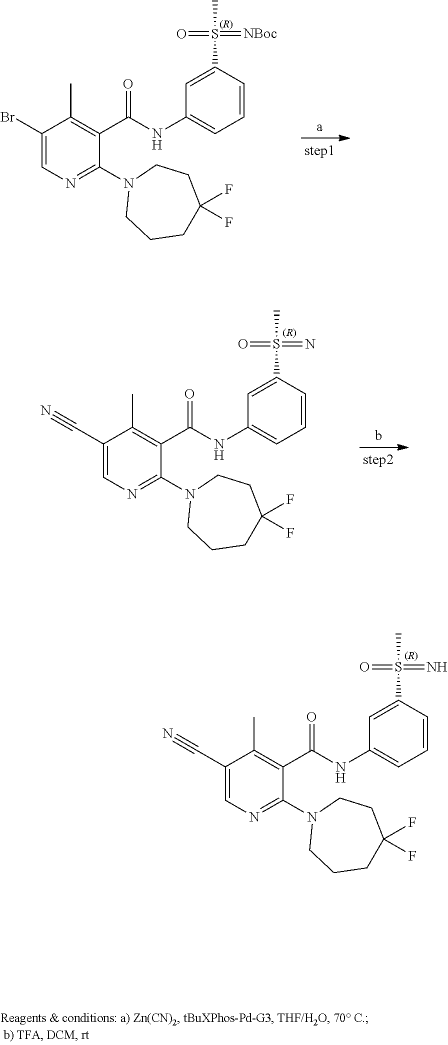

Step 1: tert-butyl (R)-((3-(5-cyano-2-(4,4-difluoroazepan-1-yl)-4-methylnicotinamido)phenyl)(methyl)(oxo)-λ⁶-sulfaneylidene)carbamate: A mixture of tert-butyl (R)-((3-(5-bromo-2-(4,4-difluoroazepan-1-yl)-4-methylnicotinamido)phenyl)(methyl)(oxo)-λ⁶-sulfaneylidene)carbamate (100 mg, 0.167 mmol), Zn(CN)₂ (39 mg, 0.333 mmol), tBuXPhos-Pd-G3 (14 mg, 0.017 mmol) in THF/H₂O (4/1, 5 mL) was heated at 70° C. for 16 h under an atmosphere of N₂. After the reaction was completed, the mixture was cooled to room temperature. The resulting solution was diluted with water (30 mL) and extracted with EtOAc (30 mL×3). The combined organic phases were washed with brine, dried over Na₂SO₄, concentrated under vacuum. The residue was purified by flash column chromatography on silica gel (PE/EtOAc=1/1) to give tert-butyl (R)-((3-(5-cyano-2-(4,4-difluoroazepan-1-yl)-4-methylnicotinamido)phenyl)(methyl)(oxo)-λ⁶-sulfaneylidene)carbamate (60 mg, 65.93%) as a yellow oil. LCMS (ESI) calcd. for C₂₆H₃₂F₂N₅O₄S [M+H]⁺ m/z 548.21, found 548.15.

Step 2: (R)-5-cyano-2-(4,4-difluoroazepan-1-yl)-4-methyl-N-(3-(S-methylsulfonimidoyl)phenyl)nicotinamide: A solution of tert-butyl (R)-((3-(5-cyano-2-(4,4-difluoroazepan-1-yl)-4-methylnicotinamido)phenyl)(methyl)(oxo)-λ⁶-sulfaneylidene)carbamate (60 mg, 0.109 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 45% to 95% MeCN/H₂O containing 0.1% formic acid) to provide (R)-5-cyano-2-(4,4-difluoroazepan-1-yl)-4-methyl-N-(3-(S-methylsulfonimidoyl)phenyl)nicotinamide (38.5 mg, 77.55%) as a white solid. ¹H NMR (400 MHz, DMSO-d₆) δ 10.91 (s, 1H), 8.56 (s, 1H), 8.29 (s, 1H), 7.89 (d, J=8.1 Hz, 1H), 7.69 (d, J=8.0 Hz, 1H), 7.60 (t, J=7.9 Hz, 1H), 4.24 (s, 1H), 3.74 (s, 2H), 3.62 (t, J=5.8 Hz, 2H), 3.07 (s, 3H), 2.37 (s, 3H), 2.35-2.21 (m, 2H), 2.07-1.93 (m, 2H), 1.88-1.79 (m, 2H). LCMS (ESI) calcd. for C₂₁H₂₄F₂N₅O₂S [M+H]⁺ m/z 448.16, found 448.00.

### Example 126

### (R)-5-cyano-2-(4,4-difluoroazepan-1-yl)-4-methyl-N-(3-(S-methyl-N-(methylglycyl)sulfonimidoyl)phenyl)nicotinamide

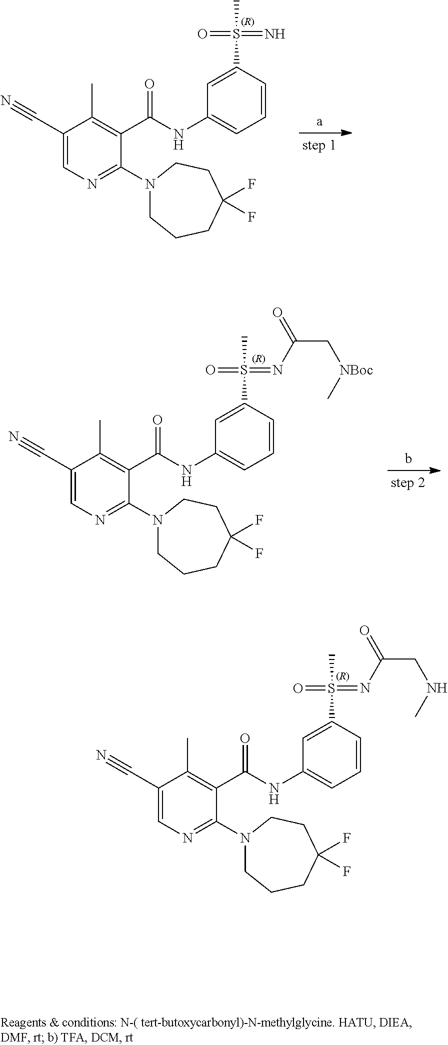

Step 1: tert-butyl (R)-(2-(((3-(5-cyano-2-(4,4-difluoroazepan-1-yl)-4-methylnicotinamido)phenyl)(methyl)(oxo)-λ⁶-sulfaneylidene)amino)-2-oxoethyl)(methyl)carbamate: A mixture of (R)-5-cyano-2-(4,4-difluoroazepan-1-yl)-4-methyl-N-(3-(S-methylsulfonimidoyl)phenyl)nicotinamide (110 mg, 0.246 mmol), N-(tert-butoxycarbonyl)-N-methylglycine (70 mg, 0.369 mmol), HATU (187 mg, 0.492 mmol) and DIEA (95 mg, 0.738 mmol) in DMF (3 mL) was stirred at room temperature for 16 hours. After the reaction was completed, the mixture was cooled to room temperature. The resulting solution was diluted with water (30 mL) and extracted with EtOAc (30 mL×3). The combined organic phases were washed with brine, dried over Na₂SO₄, concentrated under vacuum. The residue was purified by flash column chromatography on silica gel (PE/EtOAc=2/1) to give tert-butyl (R)-(2-(((3-(5-cyano-2-(4,4-difluoroazepan-1-yl)-4-methylnicotinamido)phenyl)(methyl)(oxo)-λ⁶-sulfaneylidene)amino)-2-oxoethyl)(methyl)carbamate (90 mg, 59.21%) as a yellow oil. LCMS (ESI) calcd. for C₂₉H₃₇F₂N₆O₅S [M+H]⁺ m/z 619.25, found 619.20.

Step 2: (R)-5-cyano-2-(4,4-difluoroazepan-1-yl)-4-methyl-N-(3-(S-methyl-N-(methylglycyl)sulfonimidoyl)phenyl)nicotinamide: A solution of tert-butyl (R)-(2-(((3-(5-cyano-2-(4,4-difluoroazepan-1-yl)-4-methylnicotinamido)phenyl)(methyl)(oxo)-λ⁶-sulfaneylidene)amino)-2-oxoethyl)(methyl)carbamate (90 mg, 0.145 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 45% to 95% MeCN/H₂O containing 0.1% formic acid) to provide (R)-5-cyano-2-(4,4-difluoroazepan-1-yl)-4-methyl-N-(3-(S-methyl-N-(methylglycyl)sulfonimidoyl)phenyl)nicotinamide (62 mg, 82.12%) as a white solid. ¹H NMR (400 MHz, DMSO-d₆) δ 11.09 (s, 1H), 8.56 (s, 1H), 8.34 (s, 1H), 8.24 (s, 1H), 7.97 (d, J=7.7 Hz, 1H), 7.69 (dt, J=15.6, 7.9 Hz, 2H), 3.74 (s, 2H), 3.61 (t, J=5.8 Hz, 2H), 3.50 (s, 3H), 3.41 (s, 2H), 2.37 (s, 3H), 2.33 (s, 3H), 2.31-2.22 (m, 2H), 2.06-1.95 (m, 2H), 1.89-1.79 (m, 2H). LCMS (ESI) calcd. for C₂₄H₂₉F₂N₆O₃S [M+H]⁺ m/z 519.20, found 519.15.

### Example 127

### (R)-2-(4,4-difluoroazepan-1-yl)-4-methyl-N-(3-(S-methylsulfonimidoyl)phenyl)-5-(prop-1-yn-1-yl)nicotinamide

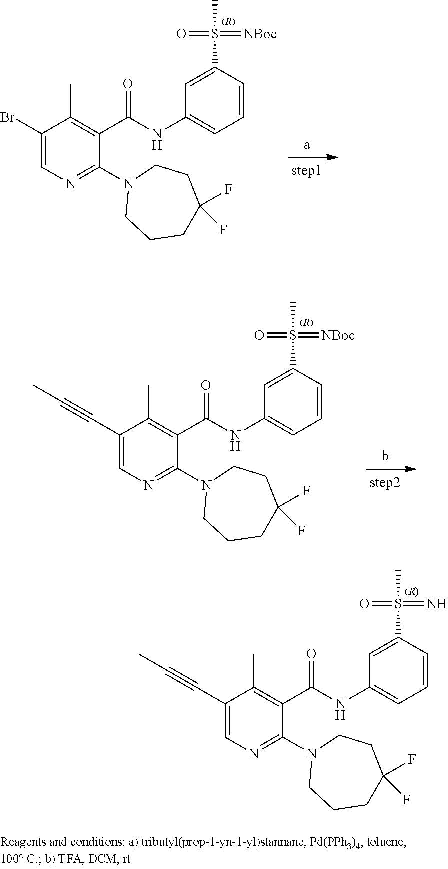

Step 1: tert-butyl (R)-((3-(2-(4,4-difluoroazepan-1-yl)-4-methyl-5-(prop-1-yn-1-yl)nicotinamido)phenyl)(methyl)(oxo)-λ⁶-sulfaneylidene)carbamate: A mixture of tert-butyl (R)-((3-(5-bromo-2-(4,4-difluoroazepan-1-yl)-4-methylnicotinamido)phenyl)(methyl)(oxo)-λ⁶-sulfaneylidene)carbamate (120 mg, 0.199 mmol), tributyl(prop-1-yn-1-yl)stannane (79 mg, 0.239 mmol) and Pd(PPh₃)₄ (23.0 mg, 0.02 mmol) in toluene (6 mL) was heated at 100° C. for 16 h under an atmosphere of N₂. After the reaction was completed, the mixture was cooled to room temperature. The resulting solution was diluted with water (30 mL) and extracted with EtOAc (30 mL×3). The combined organic phases were washed with brine, dried over Na₂SO₄, concentrated under vacuum. The residue was purified by flash column chromatography on silica gel (PE/EtAOc=1/1) to give tert-butyl (R)-((3-(2-(4,4-difluoroazepan-1-yl)-4-methyl-5-(prop-1-yn-1-yl)nicotinamido)phenyl)(methyl)(oxo)-λ⁶-sulfaneylidene)carbamate (70 mg, 62.5%) as a yellow oil. LCMS (ESI) calcd. for C₂₈H₃₅F₂N₄O₄S [M+H]⁺ m/z 561.23, found 561.15.

Step 2: (R)-2-(4,4-difluoroazepan-1-yl)-4-methyl-N-(3-(S-methylsulfonimidoyl)phenyl)-5-(prop-1-yn-1-yl)nicotinamide: A solution of tert-butyl (R)-((3-(2-(4,4-difluoroazepan-1-yl)-4-methyl-5-(prop-1-yn-1-yl)nicotinamido)phenyl)(methyl)(oxo)-λ⁶-sulfaneylidene)carbamate (70 mg, 0.125 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 45% to 95% MeCN/H₂O containing 0.1% formic acid) to provide (R)-2-(4,4-difluoroazepan-1-yl)-4-methyl-N-(3-(S-methylsulfonimidoyl)phenyl)-5-(prop-1-yn-1-yl)nicotinamide (25.2 mg, 43.86%) as a white solid. ¹H NMR (400 MHz, DMSO-d₆) δ 10.83 (s, 1H), 8.34 (s, 1H), 8.19 (s, 1H), 7.88 (d, J=7.9 Hz, 1H), 7.67 (d, J=7.8 Hz, 1H), 7.58 (t, J=7.9 Hz, 1H), 4.22 (s, 1H), 3.62 (d, J=5.2 Hz, 2H), 3.55 (t, J=5.9 Hz, 2H), 3.06 (s, 3H), 2.29 (s, 3H), 2.28-2.17 (m, 2H), 2.08 (s, 3H), 2.04-1.93 (m, 2H), 1.84-1.74 (m, 2H). LCMS (ESI) calcd. for C₂₃H₂₇F₂N₄O₂S [M+H]⁺ m/z 461.18, found 461.05.

### Example 128

### N-(3-carbamoylbicyclo[1.1.1]pentan-1-yl)-3-(4,4-difluoroazepan-1-yl)-5-methyl-6-(trifluoromethyl)pyridazine-4-carboxamide

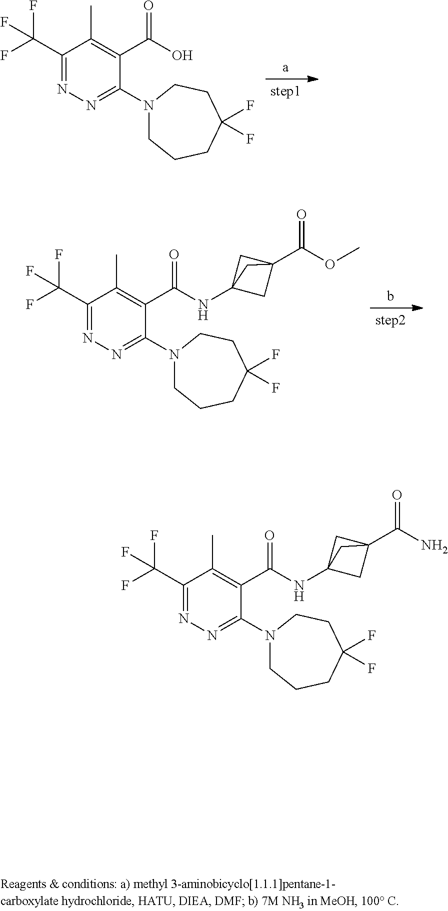 [SMILES: Cc1c(C(F)(F)F)nnc(N2CCCC(F)(F)CC2)c1C(=O)O ; COC(=O)C12CC(NC(=O)c3c(N4CCCC(F)(F)CC4)nnc(C(F)(F)F)c3C)(C1)C2 ; Cc1c(C(F)(F)F)nnc(N2CCCC(F)(F)CC2)c1C(=O)NC12CC(C(N)=O)(C1)C2]

Step 1: methyl 3-(3-(4,4-difluoroazepan-1-yl)-5-methyl-6-(trifluoromethyl)pyridazine-4-carboxamido)bicyclo[1.1.1]pentane-1-carboxylate: A solution of 3-(4,4-difluoroazepan-1-yl)-5-methyl-6-(trifluoromethyl)pyridazine-4-carboxylic acid (200 mg, 0.59 mmol), methyl 3-aminobicyclo[1.1.1]pentane-1-carboxylate hydrochloride (125.8 mg, 0.71 mmol) and HATU (336 mg, 0.89 mmol) in DMF (5 mL) was added DIEA (228 mg, 1.77 mmol) at room temperature. The reaction mixture was stirred at room temperature for 2 hours. After the reaction was completed, the resulting solution was diluted with water (30 mL) and extracted with EtOAc (30 mL×3). The combined organic phases were washed with brine, dried over Na₂SO₄, concentrated under vacuum. The residue was purified by flash column chromatography on silica gel (PE/EtOAc=1/1) to give methyl 3-(3-(4,4-difluoroazepan-1-yl)-5-methyl-6-(trifluoromethyl)pyridazine-4-carboxamido)bicyclo[1.1.1]pentane-1-carboxylate (110 mg, 40.3%) as a yellow oil. LCMS (ESI) calcd. for C₂₀H₂₄F₅N₄O₃ [M+H]⁺ m/z 463.18, found 463.15.

Step 2: N-(3-carbamoylbicyclo[1.1.1]pentan-1-yl)-3-(4,4-difluoroazepan-1-yl)-5-methyl-6-(trifluoromethyl)pyridazine-4-carboxamide: A solution of methyl 3-(3-(4,4-difluoroazepan-1-yl)-5-methyl-6-(trifluoromethyl)pyridazine-4-carboxamido)bicyclo[1.1.1]pentane-1-carboxylate (110 mg, 0.24 mmol) in 7M NH₃ MeOH solution (5 mL) was heated at 100° C. for 8 hours in a sealed tube. After the reaction was completed, the mixture was concentrated. The residue was purified by prep-HPLC (Gemini 5 um C₁₈ column, 150*21.2 mm, eluting with 30% to 80% MeCN/H₂O containing 0.1% formic acid) to provide N-(3-carbamoylbicyclo[1.1.1]pentan-1-yl)-3-(4,4-difluoroazepan-1-yl)-5-methyl-6-(trifluoromethyl)pyridazine-4-carboxamide(56.6 mg, 52.6%) as a white solid. ¹H NMR (400 MHz, DMSO-d₆) δ 9.38 (s, 1H), 7.31 (s, 1H), 7.02 (s, 1H), 3.84-3.76 (m, 2H), 3.68 (t, J=6.0 Hz, 2H), 2.39-2.28 (m, 2H), 2.24 (d, J=1.2 Hz, 3H), 2.21 (s, 6H), 2.13-2.02 (m, 2H), 1.95-1.87 (m, 2H). LCMS (ESI) calcd. for C₁₉H₂₃F₅N₅O₂ [M+H]⁺ m/z 448.18, found 448.05.

### Example 129

### (R)-2-(4,4-difluoroazepan-1-yl)-4-methyl-N₃-(3-(S-methylsulfonimidoyl)phenyl)pyridine-3,5-dicarboxamide

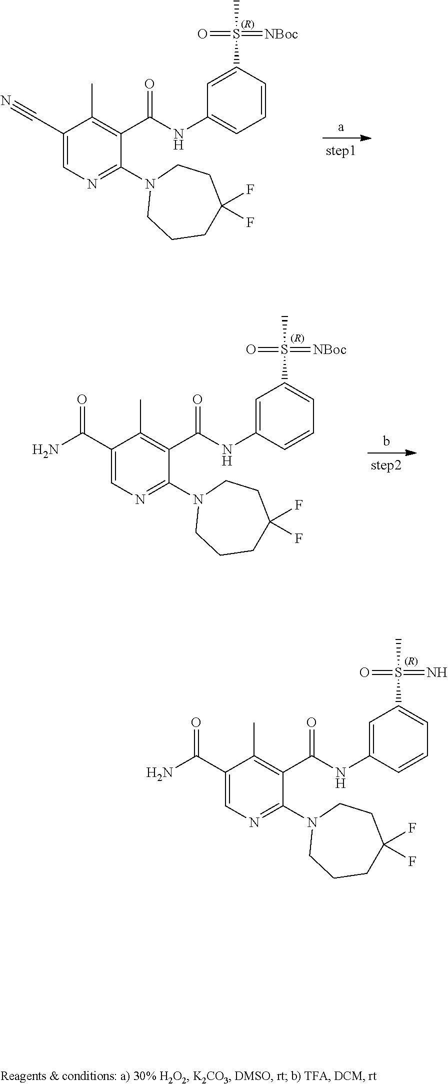

Step 1: tert-butyl (R)-((3-(5-carbamoyl-2-(4,4-difluoroazepan-1-yl)-4-methylnicotinamido)phenyl)(methyl)(oxo)-λ⁶-sulfaneylidene)carbamate: A mixture of tert-butyl (R)-((3-(5-cyano-2-(4,4-difluoroazepan-1-yl)-4-methylnicotinamido)phenyl)(methyl)(oxo)-λ⁶-sulfaneylidene)carbamate (100 mg, 0.183 mmol), K₂CO₃ (76 mg, 0.548 mmol) and 30% H₂O₂ (2 mL) in DMSO (5 mL) was stirred at room temperature for 4 h. After the reaction was completed, the mixture was cooled to room temperature. The resulting solution was diluted with water (30 mL) and extracted with EtOAc (30 mL×3). The combined organic phases were washed with brine, dried over Na₂SO₄, concentrated under vacuum. The residue was purified by flash column chromatography on silica gel (PE/EtOAc=1/5) to give tert-butyl (R)-((3-(5-carbamoyl-2-(4,4-difluoroazepan-1-yl)-4-methylnicotinamido)phenyl)(methyl)(oxo)-λ⁶-sulfaneylidene)carbamate (92 mg, 87.62%) as a yellow oil. LCMS (ESI) calcd. for C₂₆H₃₄F₂N₅O₅S [M+H]⁺ m/z 566.22, found 566.20.

Step 2: (R)-2-(4,4-difluoroazepan-1-yl)-4-methyl-N₃-(3-(S-methylsulfonimidoyl)phenyl) pyridine-3,5-dicarboxamide: A solution of tert-butyl (R)-((3-(5-carbamoyl-2-(4,4-difluoroazepan-1-yl)-4-methylnicotinamido)phenyl)(methyl)(oxo)-λ⁶-sulfaneylidene)carbamate (92 mg, 0.163 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 45% to 95% MeCN/H₂O containing 0.1% formic acid) to provide (R)-2-(4,4-difluoroazepan-1-yl)-4-methyl-N₃-(3-(S-methylsulfonimidoyl)phenyl)pyridine-3,5-dicarboxamide (61 mg, 80.58%) as a white solid. ¹H NMR (400 MHz, DMSO-d₆) δ 10.87 (s, 1H), 8.34 (s, 1H), 8.26 (s, 1H), 7.89 (d, J=8.1 Hz, 1H), 7.77 (s, 1H), 7.67 (d, J=7.8 Hz, 1H), 7.58 (t, J=7.9 Hz, 1H), 7.34 (s, 1H), 4.23 (s, 1H), 3.65 (s, 2H), 3.57 (t, J=5.9 Hz, 2H), 3.06 (s, 3H), 2.32 (s, 3H), 2.31-2.22 (m, 2H), 2.05-1.92 (m, 2H), 1.86-1.77 (m, 2H). LCMS (ESI) calcd. for C₂₁H₂₆F₂N₅O₃S [M+H]⁺ m/z 466.17, found 466.15.

### Example 130

### (R)-5-bromo-2-(4,4-difluoroazepan-1-yl)-4-methyl-N-(3-(S-methylsulfonimidoyl)phenyl)-6-(trifluoromethyl)nicotinamide

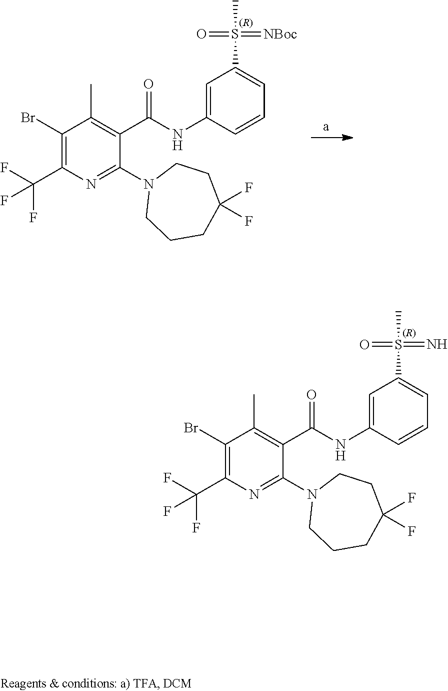

A solution of tert-butyl (R)-((3-(5-bromo-2-(4,4-difluoroazepan-1-yl)-4-methyl-6-(trifluoromethyl)nicotinamido)phenyl)(methyl)(oxo)-λ⁶-sulfaneylidene)carbamate (100 mg, 0.150 mmol) in DCM (5 mL) was added TFA (0.5 mL) at room temperature. The reaction mixture was stirred at room temperature for 2 hours. After the reaction was completed, the solution was diluted with water (10 mL) and extracted with DCM (10 mL×3). The combined organic layers were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.1% NH₃·H₂O) to obtain (R)-5-bromo-2-(4,4-difluoroazepan-1-yl)-4-methyl-N-(3-(S-methylsulfonimidoyl)phenyl)-6-(trifluoromethyl)nicotinamide (30.51 mg, 34.94%) as a white solid. ¹H NMR (400 MHz, DMSO-d₆) δ 10.98 (s, 1H), 8.29 (t, J=2.0 Hz, 1H), 7.91-7.85 (m, 1H), 7.70 (dt, J=8.0, 1.3 Hz, 1H), 7.61 (t, J=7.9 Hz, 1H), 4.32-4.20 (m, 1H), 3.67-3.52 (m, 4H), 3.07 (s, 3H), 2.40 (s, 3H), 2.33-2.23 (m, 2H), 2.09-1.95 (m, 2H), 1.86-1.77 (m, 2H). LCMS (ESI) calcd. for C₂₁H₂₃BrF₅N₄O₂S [M+H]⁺ m/z 571.06, found 571.05.

### Example 131

### (R)-5-(4-cyanophenyl)-2-(4,4-difluoroazepan-1-yl)-4-methyl-N-(3-(S-methylsulfonimidoyl)phenyl)-6-(trifluoromethyl)nicotinamide

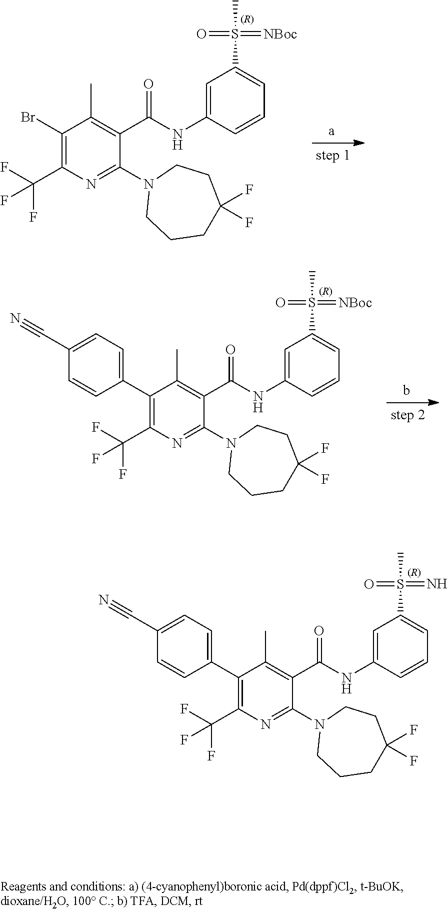

Step 1: tert-butyl (R)-((3-(5-(4-cyanophenyl)-2-(4,4-difluoroazepan-1-yl)-4-methyl-6-(trifluoromethyl)nicotinamido)phenyl)(methyl)(oxo)-λ⁶-sulfaneylidene)carbamate: A mixture of tert-butyl (R)-((3-(5-bromo-2-(4,4-difluoroazepan-1-yl)-4-methyl-6-(trifluoromethyl)nicotinamido)phenyl)(methyl)(oxo)-λ⁶-sulfaneylidene)carbamate (150 mg, 0.224 mmol), (4-cyanophenyl)boronic acid (0.098 g, 0.672 mmol), t-BuOK (51 mg, 0.448 mmol), Pd(dppf)Cl₂ (17 mg, 0.022 mmol) in dioxane/H₂O (4/1, 10 mL) was heated at 100° C. for 16 h under an atmosphere of N₂. After the reaction was completed, the mixture was cooled to room temperature. The resulting solution was diluted with water (30 mL) and extracted with EtOAc (30 mL×3). The combined organic phases were washed with brine, dried over Na₂SO₄, concentrated under vacuum. The residue was purified by flash column chromatography on silica gel (PE/EtOAc=1/1) to give tert-butyl (R)-((3-(5-(4-cyanophenyl)-2-(4,4-difluoroazepan-1-yl)-4-methyl-6-(trifluoromethyl)nicotinamido)phenyl)(methyl)(oxo)-λ⁶-sulfaneylidene)carbamate (70 mg, 45.1%) as a yellow solid. LCMS (ESI) calcd. for C₃₃H₃₄F₅N₅O₄SNa [M+Na]⁺ m/z 714.22, found 714.15.

Step 2: (R)-5-(4-cyanophenyl)-2-(4,4-difluoroazepan-1-yl)-4-methyl-N-(3-(S-methylsulfonimidoyl)phenyl)-6-(trifluoromethyl)nicotinamide: A solution of tert-butyl (R)-((3-(5-(4-cyanophenyl)-2-(4,4-difluoroazepan-1-yl)-4-methyl-6-(trifluoromethyl)nicotinamido)phenyl)(methyl)(oxo)-λ⁶-sulfaneylidene)carbamate (70 mg, 0.109 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 35% to 80% MeCN/H₂O containing 0.1% formic acid) to provide (R)-5-(4-cyanophenyl)-2-(4,4-difluoroazepan-1-yl)-4-methyl-N-(3-(S-methylsulfonimidoyl)phenyl)-6-(trifluoromethyl)nicotinamide (21.3 mg, 35.9%) as a white solid. ¹H NMR (400 MHz, DMSO-d₆) δ 11.03 (s, 1H), 8.34 (s, 1H), 7.96 (d, J=8.6 Hz, 2H), 7.89 (d, J=8.1 Hz, 1H), 7.69 (d, J=8.0 Hz, 1H), 7.60 (t, J=7.9 Hz, 1H), 7.45 (d, J=7.0 Hz, 2H), 3.73-3.61 (m, 4H), 3.07 (s, 3H), 2.37-2.26 (m, 2H), 2.11-1.98 (m, 2H), 1.91 (s, 3H), 1.88-1.79 (m, 2H). LCMS (ESI) calcd. for C₂₈H₂₇F₅N₅O₂S [M+H]⁺ m/z 592.18, found 592.15.

### Example 132

### N-(3-carbamoylbicyclo[1.1.1]pentan-1-yl)-2-(4,4-difluoroazepan-1-yl)-4-methyl-5-(1-methyl-1H-pyrazol-4-yl)nicotinamide

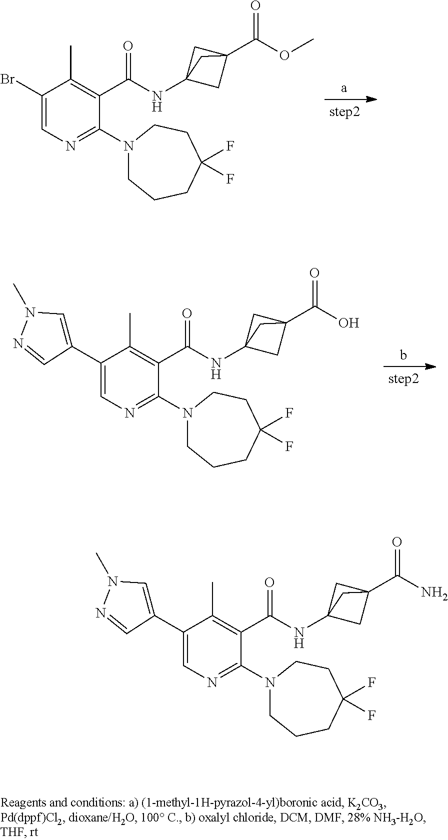

Step 1: 3-(2-(4,4-difluoroazepan-1-yl)-4-methyl-5-(1-methyl-1H-pyrazol-4-yl)nicotinamido)bicyclo[1.1.1]pentane-1-carboxylic acid: A mixture of methyl 3-(5-bromo-2-(4,4-difluoroazepan-1-yl)-4-methylnicotinamido)bicyclo[1.1.1]pentane-1-carboxylate (100 mg, 0.212 mmol), (1-methyl-1H-pyrazol-4-yl)boronic acid (40 mg, 0.318 mmol), K₂CO₃ (88 mg, 0.637 mmol) and Pd(dppf)Cl₂ (16 mg, 0.021 mmol) in dioxane/H₂O (4/1, 10 mL) was heated at 100° C. for 16 h under an atmosphere of N₂. After the reaction was completed, the mixture was cooled to room temperature. The resulting solution was diluted with water (30 mL) and extracted with EtOAc (30 mL×3). The combined organic phases were washed with brine, dried over Na₂SO₄, concentrated under vacuum. The residue was purified by flash column chromatography on silica gel (DCM/MeOH=9/1) to give 3-(2-(4,4-difluoroazepan-1-yl)-4-methyl-5-(1-methyl-1H-pyrazol-4-yl)nicotinamido)bicyclo[1.1.1]pentane-1-carboxylic acid (60 mg, 61.86%) as a yellow solid. LCMS (ESI) calcd. for C₂₃H₂₈F₂N₅O₃ [M+H]⁺ m/z 460.21, found 460.10.

Step 2: N-(3-carbamoylbicyclo[1.1.1]pentan-1-yl)-2-(4,4-difluoroazepan-1-yl)-4-methyl-5-(1-methyl-1H-pyrazol-4-yl)nicotinamide: A solution of 3-(2-(4,4-difluoroazepan-1-yl)-4-methyl-5-(1-methyl-1H-pyrazol-4-yl)nicotinamido)bicyclo[1.1.1]pentane-1-carboxylic acid (60 mg, 0.130 mmol) in DCM (6 mL) and DMF (0.02 mL) was added oxalyl chloride (33 mg, 0.261 mmol). The solution was stirred at room temperature for 30 min. Then the solution was concentrated under vacuum. The residue was diluted with THE (5 mL) and added to a stirred solution of 28% NH₃—H₂O (2 mL) in THE (5 mL). The mixture was stirred at room temperature for 3 hours. Then the mixture was quenched with water (20 mL) and extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 35% to 80% MeCN/H₂O containing 0.1% formic acid) to provide N-(3-carbamoylbicyclo[1.1.1]pentan-1-yl)-2-(4,4-difluoroazepan-1-yl)-4-methyl-5-(1-methyl-1H-pyrazol-4-yl)nicotinamide (21.4 mg, 35.67%) as a white solid. ¹H NMR (400 MHz, DMSO-d₆) δ 9.02 (s, 1H), 8.06 (s, 1H), 7.85 (s, 1H), 7.56 (s, 1H), 7.28 (s, 1H), 6.99 (s, 1H), 3.87 (s, 3H), 3.61-3.46 (m, 4H), 2.29-2.14 (m, 11H), 2.10-1.97 (m, 2H), 1.86-1.80 (m, 2H). LCMS (ESI) calcd. for C₂₃H₂₉F₂N₆O₂ [M+H]⁺ m/z 459.23, found 459.10.

### Example 133

### (R)-2-(4,4-difluoroazepan-1-yl)-4-methyl-5-(1-methyl-1H-pyrazol-4-yl)-N-(3-(S-methylsulfonimidoyl)phenyl)-6-(trifluoromethyl)nicotinamide

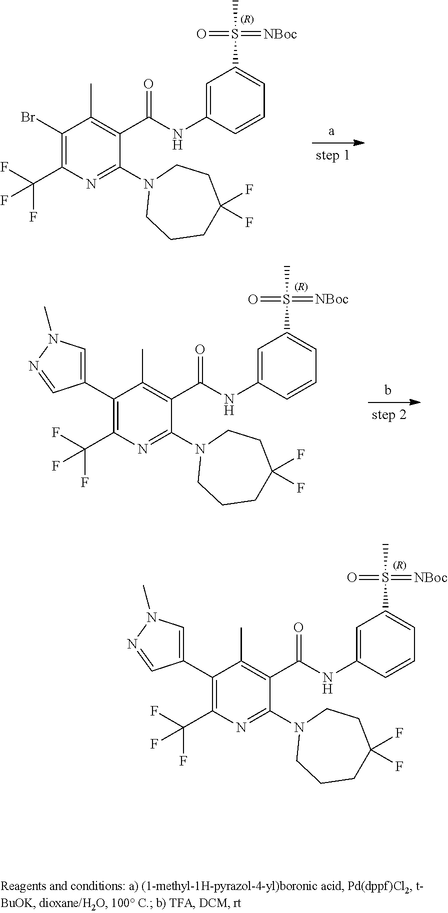

Step 1: tert-butyl (R)-((3-(2-(4,4-difluoroazepan-1-yl)-4-methyl-5-(1-methyl-1H-pyrazol-4-yl)-6-(trifluoromethyl)nicotinamido)phenyl)(methyl)(oxo)-λ⁶-sulfaneylidene)carbamate: A mixture of tert-butyl (R)-((3-(5-bromo-2-(4,4-difluoroazepan-1-yl)-4-methyl-6-(trifluoromethyl)nicotinamido)phenyl)(methyl)(oxo)-λ⁶-sulfaneylidene)carbamate (200 mg, 0.299 mmol) and (1-methyl-1H-pyrazol-4-yl)boronic acid (188 mg, 1.50 mmol) in 1,4-dioxane/H₂O (4/1, 10 mL), cesium carbonate (195 mg, 0.598 mmol) and [1,1′-Bis(diphenylphosphino)ferrocene]dichloropalladium (II) (22 mg, 0.030 mmol) was heated at 100° C. for 6 hours under an atmosphere of N₂. The reaction was monitored by LCMS. After the reaction was completed, the mixture was cooled to room temperature. The resulting solution was diluted with water (20 mL) and extracted with DCM (20 mL×3). The combined organic phases were washed with brine, dried over Na₂SO₄, concentrated under vacuum. The residue was purified by flash column chromatography on silica gel (PE/EtOAc=2/1 to 1/1) to give tert-butyl (R)-((3-(2-(4,4-difluoroazepan-1-yl)-4-methyl-5-(1-methyl-1H-pyrazol-4-yl)-6-(trifluoromethyl)nicotinamido)phenyl)(methyl)(oxo)-λ⁶--sulfaneylidene)carbamate (150 mg, 75.00%) as a yellow oil. LCMS (ESI) calcd. for C₃₀H₃₆F₅N₆O₄S [M+H]⁺ m/z 671.24, found 671.20.

Step 2: (R)-2-(4,4-difluoroazepan-1-yl)-4-methyl-5-(1-methyl-1H-pyrazol-4-yl)-N-(3-(S-methylsulfonimidoyl)phenyl)-6-(trifluoromethyl)nicotinamide: A solution of tert-butyl (R)-((3-(2-(4,4-difluoroazepan-1-yl)-4-methyl-5-(1-methyl-1H-pyrazol-4-yl)-6-(trifluoromethyl)nicotinamido)phenyl)(methyl)(oxo)-λ⁶-sulfaneylidene)carbamate (120 mg, 0.179 mmol) in DCM (5 mL) was added TFA (0.5 mL) at room temperature. The reaction mixture was stirred at room temperature for 2 hours. After the reaction was completed, the solution was diluted with water (10 mL) and extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.1% formic acid) to obtain (R)-2-(4,4-difluoroazepan-1-yl)-4-methyl-5-(1-methyl-1H-pyrazol-4-yl)-N-(3-(S-methylsulfonimidoyl)phenyl)-6-(trifluoromethyl)nicotinamide (44.21 mg, 43.13%) as a white solid. ¹H NMR (400 MHz, DMSO-d₆) δ 10.98 (s, 1H), 8.35 (s, 1H), 7.88 (d, J=8.0 Hz, 1H), 7.71 (s, 1H), 7.68 (d, J=8.0 Hz, 1H), 7.58-7.60 (m, 1H), 7.36 (s, 1H), 4.24 (s, 1H), 3.89 (s, 3H), 3.68-3.66 (m, 2H), 3.62-3.59 (m, 2H), 3.06 (s, 3H), 2.31-2.29 (m, 2H), 2.03 (s, 3H), 1.99-1.97 (m, 2H), 1.84-1.82 (m, 2H). LCMS (ESI) calcd. for C₂₅H₂₈F₅N₆O₂S [M+H]⁺ m/z 571.19, found 571.20.

### Example 134

### N-(3-carbamoylbicyclo[1.1.1]pentan-1-yl)-5-(4-cyanophenyl)-2-(4,4-difluoroazepan-1-yl)-4-methylnicotinamide

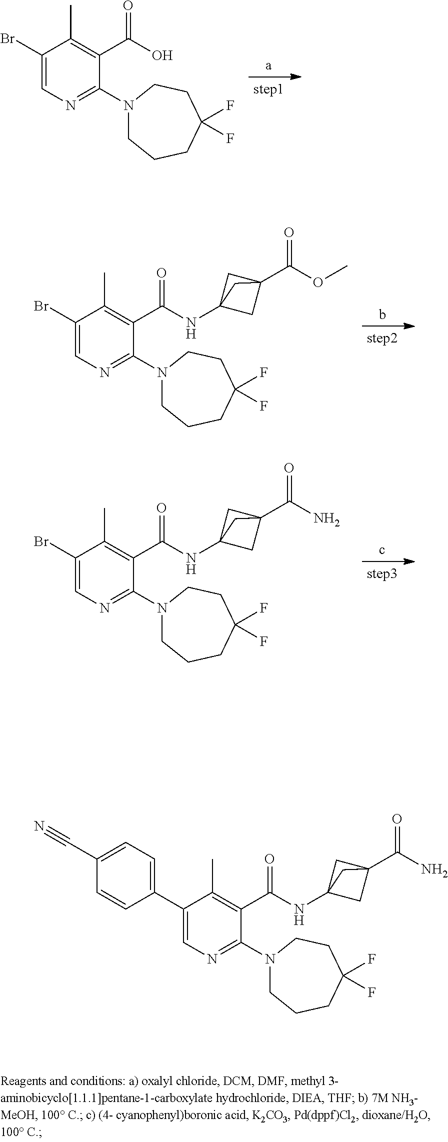 [SMILES: Cc1c(Br)cnc(N2CCCC(F)(F)CC2)c1C(=O)O ; COC(=O)C12CC(NC(=O)c3c(N4CCCC(F)(F)CC4)ncc(Br)c3C)(C1)C2 ; Cc1c(Br)cnc(N2CCCC(F)(F)CC2)c1C(=O)NC12CC(C(N)=O)(C1)C2 ; Cc1c(-c2ccc(C#N)cc2)cnc(N2CCCC(F)(F)CC2)c1C(=O)NC12CC(C(N)=O)(C1)C2]

Step 1: methyl 3-(5-bromo-2-(4,4-difluoroazepan-1-yl)-4-methylnicotinamido)bicyclo[1.1.1]pentane-1-carboxylate: A solution of 5-bromo-2-(4,4-difluoroazepan-1-yl)-4-methylnicotinic acid (700 mg, 2.01 mmol) in DCM (10 mL) and DMF (0.05 mL) was added oxalyl chloride (383 mg, 3.17 mmol). The solution was stirred at room temperature for 30 min. Then the solution was concentrated under vacuum. The residue was diluted with THE (5 mL) and added to a stirred solution of methyl 3-aminobicyclo[1.1.1]pentane-1-carboxylate hydrochloride (427 mg, 2.41 mmol) and DIEA (778 mg, 6.03 mmol) in THE (5 mL). The mixture was heated at 50° C. for 1 h. Then the mixture was quenched with water (20 mL) and extracted with DCM (20 mL×3). The combined organic phases were washed with brine, dried over sodium sulfate, concentrated under vacuum. The residue was purified by flash column chromatography on silica gel (PE/EtOAc=1/4) to provide methyl 3-(5-bromo-2-(4,4-difluoroazepan-1-yl)-4-methylnicotinamido)bicyclo[1.1.1]pentane-1-carboxylate (750 mg, 79.20%) as a yellow solid. LCMS (ESI) calcd. for C₂₀H₂₅BrF₂N₃O₃ [M+H]⁺ m/z 474.10, found 475.05.

Step 2: 5-bromo-N-(3-carbamoylbicyclo[1.1.1]pentan-1-yl)-2-(4,4-difluoroazepan-1-yl)-4-methylnicotinamide: A mixture of methyl 3-(5-bromo-2-(4,4-difluoroazepan-1-yl)-4-methylnicotinamido)bicyclo[1.1.1]pentane-1-carboxylate(750 mg, 1.59 mmol) in 7M NH₃-MeOH (10 mL) was heated at 100° C. for 8 hours in a sealed tube. After the reaction was completed, the mixture was concentrated under vacuum. The residue was purified by flash column chromatography on silica gel (PE/EtAOc=1/4) to give 5-bromo-N-(3-carbamoylbicyclo[1.1.1]pentan-1-yl)-2-(4,4-difluoroazepan-1-yl)-4-methylnicotinamide (700 mg, 96.42%) as a yellow solid. LCMS (ESI) calcd. for C₁₉H₂₄BrF₂N₄O₂ [M+H]⁺ m/z 459.10, found 458.95.

Step 3: N-(3-carbamoylbicyclo[1.1.1]pentan-1-yl)-5-(4-cyanophenyl)-2-(4,4-difluoroazepan-1-yl)-4-methylnicotinamide: A solution of 5-bromo-N-(3-carbamoylbicyclo[1.1.1]pentan-1-yl)-2-(4,4-difluoroazepan-1-yl)-4-methylnicotinamide (120 mg, 0.26 mmol), (4-cyanophenyl)boronic acid (76 mg, 0.52 mmol), Pd(dppf)Cl₂ (19 mg, 0.026 mmol) and K₂CO₃ (108 mg, 0.78 mmol) in dioxane (2 mL) and H₂O (0.5 mL) was heated at 100° C. for 12 h under N₂ atmosphere. Then the mixture was diluted with water and extracted with EtOAc (3×10 mL). The combined organic phases were washed with brine, dried with Na₂SO₄, and concentrated under vacuum. The residue was purified by prep-HPLC (Gemini 5 um C₁₈ column, 150*19 mm, eluting with 20% to 70% MeCN/H₂O containing 0.1% FA) to afford N-(3-carbamoylbicyclo[1.1.1]pentan-1-yl)-5-(4-cyanophenyl)-2-(4,4-difluoroazepan-1-yl)-4-methylnicotinamide (57.4 mg, 23.04%) as a white solid. LCMS (ESI) calcd. for C₂₆H₂₈F₂N₅O₂ [M+H]⁺ m/z 480.22, found 480.20. ¹H NMR (400 MHz, DMSO-d₆) δ 9.07 (s, 1H), 8.02 (s, 1H), 7.90 (d, J=8.2 Hz, 2H), 7.52 (d, J=8.2 Hz, 2H), 7.28 (s, 1H), 6.99 (s, 1H), 3.69-3.53 (m, 4H), 2.37-2.22 (m, 2H), 2.19 (s, 6H), 2.10-1.99 (m, 5H), 1.91-1.81 (m, 2H).

### Example 135

### (R)-4-(4,4-difluoroazepan-1-yl)-6-methyl-N-(3-(S-methylsulfonimidoyl)phenyl)-2-(trifluoromethyl)pyrimidine-5-carboxamide

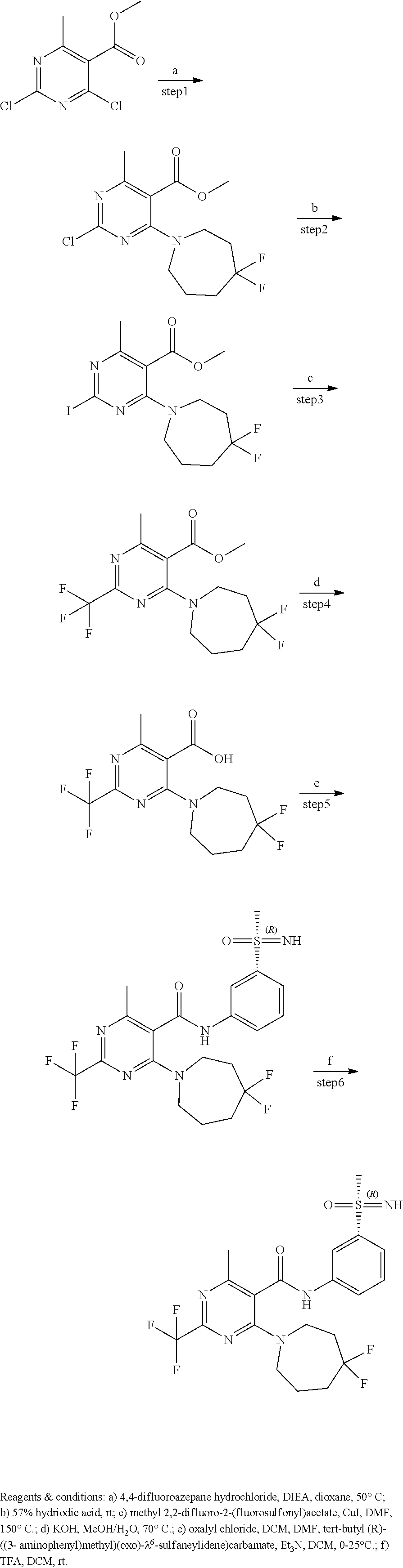

Step 1: methyl 2-chloro-4-(4,4-difluoroazepan-1-yl)-6-methylpyrimidine-5-carboxylate: A solution of methyl 2,4-dichloro-6-methylpyrimidine-5-carboxylate (3.50 g, 15.83 mmol), 4,4-difluoroazepane hydrochloride (2.71 g, 15.83 mmol) and DIPEA (5.11 g, 39.58 mmol) in dioxane (50 mL) was heated at 50° C. for 6 hours. LCMS showed the reaction was completed. The mixture was diluted with water (60 mL) and extracted with EtOAc (60 mL×3). The combined organic layers were washed with brine, dried over sodium sulfate, concentrated under vacuum. The residue was purified by flash column chromatography on silica gel (PE/EtOAc=5/1) to provide methyl 2-chloro-4-(4,4-difluoroazepan-1-yl)-6-methylpyrimidine-5-carboxylate (3.6 g, 67.55%) as white solid. LCMS (ESI) calcd. for C₁₃H₁₇ClF₂N₃O₂ [M+H]⁺ m/z 320.10, found 320.10.

Step 2: methyl 4-(4,4-difluoroazepan-1-yl)-2-iodo-6-methylpyrimidine-5-carboxylate: A solution of methyl 2-chloro-4-(4,4-difluoroazepan-1-yl)-6-methylpyrimidine-5-carboxylate (1 g, 3.13 mmol) in 57% hydriodic acid (5 mL) was stirred at 25° C. for 48 hours. LCMS showed the reaction was completed. The mixture was diluted with water (30 mL) and extracted with EtOAc (60 mL×3). The combined organic layers were washed with brine, dried over sodium sulfate, concentrated under vacuum. The residue was purified by flash column chromatography on silica gel (PE/EtOAc=5/1) to provide methyl 4-(4,4-difluoroazepan-1-yl)-2-iodo-6-methylpyrimidine-5-carboxylate (670 mg, 50.08%) as a brown solid. LCMS (ESI) calcd. for C₁₃H₁₇F₂IN₃O₂ [M+H]⁺ m/z 411.04, found 412.00.

Step 3: methyl 4-(4,4-difluoroazepan-1-yl)-6-methyl-2-(trifluoromethyl)pyrimidine-5-carboxylate: A solution of methyl 4-(4,4-difluoroazepan-1-yl)-2-iodo-6-methylpyrimidine-5-carboxylate (670 mg, 1.63 mmol), methyl 2,2-difluoro-2-(fluorosulfonyl)acetate (626 mg, 3.26 mmol) and CuI (619 mg, 3.26 mmol) in DMF (10 mL) heated at 150° C. for 2 hours under an atmosphere of N₂. LCMS showed the reaction was completed. The mixture was diluted with water (30 mL) and extracted with EtOAc (60 mL×3). The combined organic phases were washed with brine (60 mL×3), dried over sodium sulfate, concentrated under vacuum. The residue was purified by flash column chromatography on silica gel (PE/EtOAc=2/1) to provide methyl 4-(4,4-difluoroazepan-1-yl)-6-methyl-2-(trifluoromethyl)pyrimidine-5-carboxylate (380 mg, 66.26%) as a brown solid. LCMS (ESI) calcd. for C₁₄H₁₇F₅N₃O₂ [M+H]⁺ m/z 354.12, found 354.10.

Step 4: 4-(4,4-difluoroazepan-1-yl)-6-methyl-2-(trifluoromethyl)pyrimidine-5-carboxylic acid: To a solution of methyl 4-(4,4-difluoroazepan-1-yl)-6-methyl-2-(trifluoromethyl)pyrimidine-5-carboxylate (380 mg, 1.08 mmol) in MeOH/H₂O (1/1, 4 mL) was added KOH (603 mg, 10.76 mmol) at room temperature. The mixture was heated at 70° C. for 12 hours. After the reaction was completed, the mixture was cooled to room temperature and concentrated to remove most MeOH. The residue was dissolved with H₂O (10 mL), adjusted to pH=3-4 with 1N HCl and extracted with EtOAc (10 mL×3). The combined organic layers were washed with brine, dried with Na₂S₀₄, and concentrated under reduced pressure to give 4-(4,4-difluoroazepan-1-yl)-6-methyl-2-(trifluoromethyl)pyrimidine-5-carboxylic acid (310 mg, 40.74%). LCMS (ESI) calcd. for C₁₃H₁₅F₅N₃O₂ [M+H]⁺ m/z 340.11, found 340.10.

Step 5: tert-butyl (R)-((3-(4-(4,4-difluoroazepan-1-yl)-6-methyl-2-(trifluoromethyl)pyrimidine-5-carboxamido)phenyl)(methyl)(oxo)-λ⁶-sulfaneylidene)carbamate: A solution of 4-(4,4-difluoroazepan-1-yl)-6-methyl-2-(trifluoromethyl)pyrimidine-5-carboxylic acid (150 mg, 0.44 mmol) in DCM (3 mL) and DMF (one drop) was added oxalyl chloride (119 mg, 1 mmol). The solution was stirred at room temperature for 5 min. Then the solution was added to a stirred solution of tert-butyl (R)-((3-aminophenyl)(methyl)(oxo)-λ⁶-sulfaneylidene)carbamate (180 mg, 0.68 mmol) and Et₃N (141 mg, 1.39 mmol) in DCM (3 mL) at 0° C. The resulting mixture was stirred at 25° C. for 1 hour. Then the mixture was quenched with water (20 mL) and extracted with DCM (20 mL×2). The combined organic layers were washed with brine, dried over sodium sulfate, concentrated under vacuum. The residue was purified by flash column chromatography on silica gel (PE/EtOAc=1/1) to provide tert-butyl (R)-((3-(4-(4,4-difluoroazepan-1-yl)-6-methyl-2-(trifluoromethyl)pyrimidine-5-carboxamido)phenyl)(methyl)(oxo)-λ⁶-sulfaneylidene)carbamate (98 mg, 37.73%) as a brown oil. LCMS (ESI) calcd. for C₂₅H₃₁F₅N₅O₄S [M+H]⁺ m/z 592.20, found 592.15.

Step 6: (R)-4-(4,4-difluoroazepan-1-yl)-6-methyl-N-(3-(S-methylsulfonimidoyl)phenyl)-2-(trifluoromethyl)pyrimidine-5-carboxamide: A solution of tert-butyl (R)-((3-(4-(4,4-difluoroazepan-1-yl)-6-methyl-2-(trifluoromethyl)pyrimidine-5-carboxamido)phenyl)(methyl)(oxo)-λ⁶-sulfaneylidene)carbamate (98 mg, 0.166 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 35% to 80% MeCN/H₂O containing 0.1% NH₃·H₂O) to provide (R)-4-(4,4-difluoroazepan-1-yl)-6-methyl-N-(3-(S-methylsulfonimidoyl)phenyl)-2-(trifluoromethyl)pyrimidine-5-carboxamide (75.4 mg, 88.28%) as a white solid. ¹H NMR (400 MHz, DMSO-d₆) δ 11.01 (s, 1H), 8.27 (s, 1H), 7.87 (d, J=7.7 Hz, 1H), 7.72-7.55 (m, 2H), 4.24 (s, 1H), 3.85-3.62 (m, 4H), 3.07 (s, 3H), 2.41 (s, 3H), 2.28 (d, J=15.2 Hz, 2H), 2.11-1.99 (m, 2H), 1.90-1.80 (m, 2H). LCMS (ESI) calcd. for C₂₀H₂₃F₅N₅O₂S [M+H]⁺ m/z 492.15, found 492.10.

### Example 136

### N-(3-carbamoylbicyclo[1.1.1]pentan-1-yl)-5-cyano-2-(4,4-difluoroazepan-1-yl)-4-methylnicotinamide

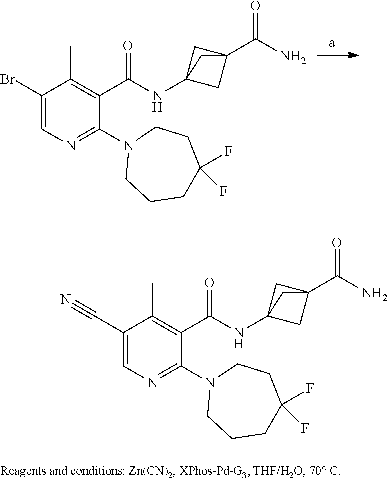 [SMILES: Cc1c(Br)cnc(N2CCCC(F)(F)CC2)c1C(=O)NC12CC(C(N)=O)(C1)C2 ; Cc1c(C#N)cnc(N2CCCC(F)(F)CC2)c1C(=O)NC12CC(C(N)=O)(C1)C2]

A mixture of 5-bromo-N-(3-carbamoylbicyclo[1.1.1]pentan-1-yl)-2-(4,4-difluoroazepan-1-yl)-4-methylnicotinamide (100 mg, 0.219 mmol), Zn(CN)₂ (51 mg, 0.439 mmol) and t-Bu-XPhos-Pd-G₃ (17 mg, 0.022 mmol) in THF/H₂O (4/1, 10 mL) was heated at 70° C. for 16 h under an atmosphere of N₂. After the reaction was completed, the mixture was cooled to room temperature. The resulting solution was diluted with water (30 mL) and extracted with EtOAc (30 mL×3). The combined organic phases were washed with brine, dried over Na₂SO₄, concentrated under vacuum. The residue was purified by prep-HPLC (Gemini 5 um C₁₈ column, 150*21.2 mm, eluting with 35% to 80% MeCN/H₂O containing 0.1% formic acid) to provide N-(3-carbamoylbicyclo[1.1.1]pentan-1-yl)-5-cyano-2-(4,4-difluoroazepan-1-yl)-4-methylnicotinamide (57.8 mg, 65.68%) as a white solid. ¹H NMR (400 MHz, DMSO-d₆) δ 9.15 (s, 1H), 8.45 (d, J=1.2 Hz, 1H), 7.29 (s, 1H), 7.00 (s, 1H), 3.74-3.59 (m, 4H), 2.32-2.19 (m, 11H), 2.08-1.97 (m, 2H), 1.90-1.82 (m, 2H). LCMS (ESI) calcd. for C₂₀H₂₄F₂N₅O₂ [M+H]⁺ m/z 404.19, found 404.05.

### Examples 137 and 138

### (S)-3-(4,4-difluoroazepan-1-yl)-6-(4-methoxyphenyl)-5-methyl-N-(3-(S-methylsulfonimidoyl)phenyl)pyridazine-4-carboxamide and (R)-3-(4,4-difluoroazepan-1-yl)-6-(4-methoxyphenyl)-5-methyl-N-(3-(S-methylsulfonimidoyl)phenyl)pyridazine-4-carboxamide

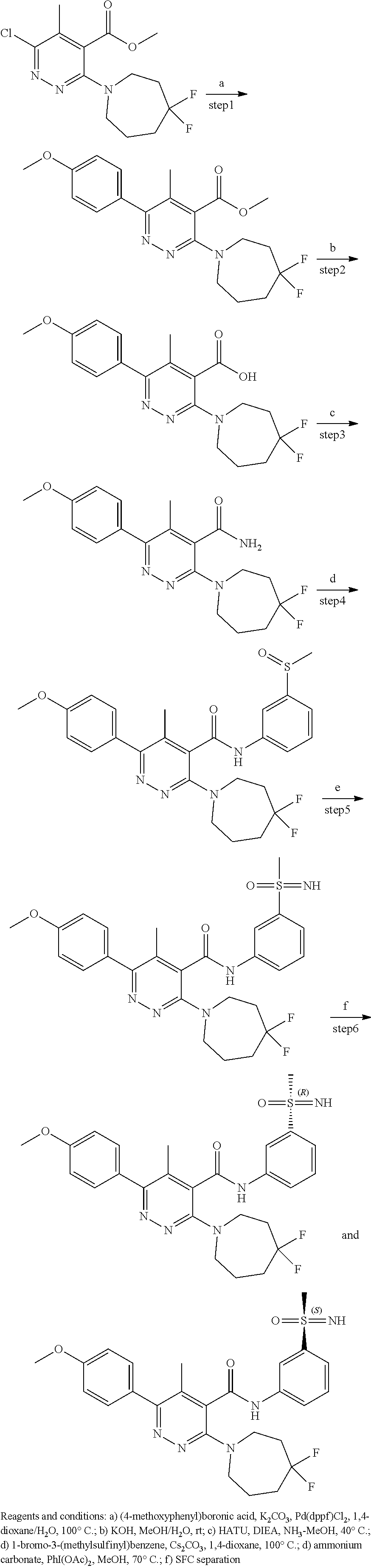

Step 1: methyl 3-(4,4-difluoroazepan-1-yl)-6-(4-methoxyphenyl)-5-methylpyridazine-4-carboxylate: A mixture of methyl 6-chloro-3-(4,4-difluoroazepan-1-yl)-5-methylpyridazine-4-carboxylate (800 mg, 2.50 mmol) and (4-methoxyphenyl)boronic acid (570 mg, 3.75 mmol) in 1,4-dioxane/H₂O (4/1, 20 mL), potassium carbonate (1036 mg, 7.50 mmol) and Pd(dppf)Cl₂·DCM (183 mg, 0.250 mmol) was heated at 100° C. for 6 h under an atmosphere of N₂. The reaction was monitored by LCMS. After the reaction was completed, the mixture was cooled to room temperature. The resulting solution was diluted with water (20 mL) and extracted with DCM (20 mL×3). The combined organic phases was washed with brine, dried over Na₂SO₄, concentrated under vacuum. The residue was purified by flash column chromatography on silica gel (PE/EtAOc=3/1) to give methyl 3-(4,4-difluoroazepan-1-yl)-6-(4-methoxyphenyl)-5-methylpyridazine-4-carboxylate (830 mg, 84.69%) as a white oil. LCMS (ESI) calcd. for C₂₀H₂₄F₂N₃O₃ [M+H]⁺ m/z 392.18, found 392.25.

Step 2: 3-(4,4-difluoroazepan-1-yl)-6-(4-methoxyphenyl)-5-methylpyridazine-4-carboxylic acid: To a solution methyl 3-(4,4-difluoroazepan-1-yl)-6-(4-methoxyphenyl)-5-methylpyridazine-4-carboxylate (800 mg, 2.04 mmol) in MeOH/H₂O (1/1, 10 mL) was added KOH (1144.64 mg, 20.40 mmol) at room temperature. The mixture was heated at 70° C. for 6 hours. After the reaction was completed, the mixture was cooled to room temperature and extracted with DCM (10 mL). Then the aqueous phase was adjusted to pH=3 with 1N HCl and extracted with DCM (10 mL×3). The combined organic phases was dried with Na₂SO₄ and concentrated under reduced pressure to give crude 3-(4,4-difluoroazepan-1-yl)-6-(4-methoxyphenyl)-5-methylpyridazine-4-carboxylic acid (670 mg, 87.13%) as a white solid. LCMS (ESI) calcd. for C₁₉H₂₂F₂N₃O₃ [M+H]⁺ m/z 378.16, found 378.10.

Step 3: 3-(4,4-difluoroazepan-1-yl)-6-(4-methoxyphenyl)-5-methylpyridazine-4-carboxamide: A solution of 3-(4,4-difluoroazepan-1-yl)-6-(4-methoxyphenyl)-5-methylpyridazine-4-carboxylic acid (650 mg, 1.719 mmol) in DMF (10 mL) was added HATU (850 mg, 2.235 mmol), DIEA (710 mg, 5.500 mmol) and NH₃-MeOH (5 mL) at room temperature. The mixture was heated at 40° C. for 16 hours. After the reaction was completed, the mixture was cooled to room temperature, diluted with water 2 mL) and extracted with DCM (20 mL×3). The combined organic phases was washed with brine, dried over sodium sulfate, concentrated under vacuum. The residue was purified by flash column chromatography on silica gel (PE/EtOAc=1/1) to give 3-(4,4-difluoroazepan-1-yl)-6-(4-methoxyphenyl)-5-methylpyridazine-4-carboxamide (500 mg, 76.92%) as a white solid. LCMS (ESI) calcd. for C₁₉H₂₃F₂N₄O₂ [M+H]⁺ m/z 377.18, found 377.05.

Step 4: 3-(4,4-difluoroazepan-1-yl)-6-(4-methoxyphenyl)-5-methyl-N-(3-(methylsulfinyl)phenyl)pyridazine-4-carboxamide: A solution of 3-(4,4-difluoroazepan-1-yl)-6-(4-methoxyphenyl)-5-methylpyridazine-4-carboxamide (450 mg, 1.194 mmol) in dioxane (8 mL) was added 1-bromo-3-(methylsulfinyl)benzene (785 mg, 3.582 mmol), Cs₂CO₃ (1011 mg, 3.104 mmol) and Xantphos-Pd-G2 (212 mg, 0.239 mmol) at room temperature. The reaction mixture was stirred at 100° C. for 16 hours under nitrogen. The reaction was monitored by LCMS. After the reaction was completed, the filtrate was diluted with water (20 mL) and extracted with DCM (20 mL×3). The combined organic phases was washed with brine, dried over sodium sulfate, concentrated under vacuum. The residue was purified by flash column chromatography on silica gel (PE/EtOAc=1/1) to give 3-(4,4-difluoroazepan-1-yl)-6-(4-methoxyphenyl)-5-methyl-N-(3-(methylsulfinyl)phenyl)pyridazine-4-carboxamide (300 mg, 48.86%) as a white solid. LCMS (ESI) calcd. for C₂₆H₂₉F₂N₄O₃S [M+H]⁺ m/z 515.19, found 515.10.

Step 5: 3-(4,4-difluoroazepan-1-yl)-6-(4-methoxyphenyl)-5-methyl-N-(3-(S-methylsulfonimidoyl)phenyl)pyridazine-4-carboxamide: To a solution of 3-(4,4-difluoroazepan-1-yl)-6-(4-methoxyphenyl)-5-methyl-N-(3-(methylsulfinyl)phenyl)pyridazine-4-carboxamide (280 mg, 0.544 mmol) in MeOH (5 mL) was added PhI(OAc)₂ (438 mg, 1.359 mmol) and ammonium carbamate (127 mg, 1.632 mmol) at room temperature. The reaction mixture was heated at 70° C. for 1 hour. After the reaction was completed, the mixture was cooled to room temperature, diluted with water (5 mL), and extracted with DCM (5 mL×3). The combined organic phases were washed with brine, dried over sodium sulfate, concentrated under vacuum. The residue was first purified by flash column chromatography on silica gel (PE/EtOAc=1/1), then further purified by prep-HPLC (Gemini 5 um C₁₈ column, 150*21.2 mm, eluting with 30% to 90% MeCN/H₂O containing 0.1% formic acid) to afford 3-(4,4-difluoroazepan-1-yl)-6-(4-methoxyphenyl)-5-methyl-N-(3-(S-methylsulfonimidoyl)phenyl)pyridazine-4-carboxamide (60 mg, 20.83%) as a white solid. LCMS (ESI) calcd. for C₂₆H₃₀F₂N₅O₃S [M+H]⁺ m/z 530.20, found 530.15.

Step 6: (S)-3-(4,4-difluoroazepan-1-yl)-6-(4-methoxyphenyl)-5-methyl-N-(3-(S-methylsulfonimidoyl)phenyl)pyridazine-4-carboxamide and (R)-3-(4,4-difluoroazepan-1-yl)-6-(4-methoxyphenyl)-5-methyl-N-(3-(S-methylsulfonimidoyl)phenyl)pyridazine-4-carboxamide: A racemic mixture of 3-(4,4-difluoroazepan-1-yl)-6-(4-methoxyphenyl)-5-methyl-N-(3-(S-methylsulfonimidoyl)phenyl)pyridazine-4-carboxamide (60 mg) was purified by Chiral SFC (chiralpak-AD-H, 150*21.2 mm, eluting with 40% CO₀₂-MeOH containing 0.1% NH₃). The first eluting isomer was lyophilized to afford (S)-3-(4,4-difluoroazepan-1-yl)-6-(4-methoxyphenyl)-5-methyl-N-(3-(S-methylsulfonimidoyl)phenyl)pyridazine-4-carboxamide (16.2 mg, 95.0% purity) as a white solid. ¹H NMR (400 MHz, DMSO-d₆) δ 11.07 (s, 1H), 8.32-8.31 (m, 1H), 7.88 (d, J=8 Hz, 1H), 7.70 (d, J=8 Hz, 1H), 7.60-7.58 (m, 1H), 7.50 (d, J=8.8 Hz, 2H), 7.06 (d, J=8.8 Hz, 2H), 4.25 (s, 1H), 3.82 (s, 3H), 3.78-3.75 (m, 2H), 3.66-3.63 (m, 2H), 3.06 (s, 3H), 2.33-2.32 (m, 2H), 2.19 (s, 3H), 2.05-2.03 (m, 2H), 1.87-1.85 (m, 2H). LCMS (ESI) calcd. for C₂₆H₃₀F₂N₅O₃S [M+H]⁺ m/z 530.20, found 530.15. The second eluting isomer was lyophilized to afford (R)-3-(4,4-difluoroazepan-1-yl)-6-(4-methoxyphenyl)-5-methyl-N-(3-(S-methylsulfonimidoyl)phenyl)pyridazine-4-carboxamide (21.5 mg) as a white solid. ¹H NMR (400 MHz, DMSO-d₆) δ 11.07 (s, 1H), 8.32-8.31 (m, 1H), 7.89 (d, J=9.2 Hz, 1H), 7.70 (d, J=8 Hz, 1H), 7.60-7.58 (m, 1H), 7.50 (d, J=8.8 Hz, 2H), 7.06 (d, J=8.8 Hz, 2H), 4.25 (s, 1H), 3.82 (s, 3H), 3.77-3.75 (m, 2H), 3.66-3.63 (m, 2H), 3.06 (s, 3H), 2.33-2.32 (m, 2H), 2.19 (s, 3H), 2.05-2.03 (m, 2H), 1.87-1.85 (m, 2H). LCMS (ESI) calcd. for C₂₆H₃₀F₂N₅O₃S [M+H]⁺ m/z 530.20, found 530.15.

### Example 139

### (R)-6-cyano-3-(4,4-difluoroazepan-1-yl)-5-methyl-N-(3-(S-methylsulfonimidoyl)phenyl)pyridazine-4-carboxamide

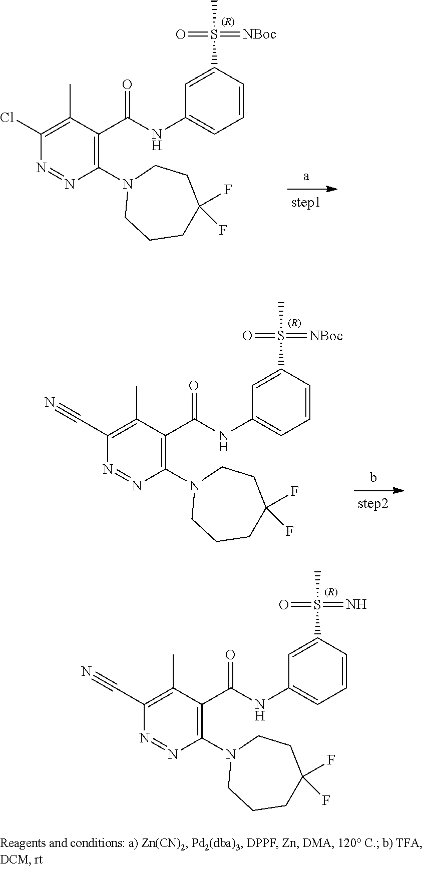

Step 1: tert-butyl (R)-((3-(6-cyano-3-(4,4-difluoroazepan-1-yl)-5-methylpyridazine-4-carboxamido)phenyl)(methyl)(oxo)-λ⁶-sulfaneylidene)carbamate: A mixture of tert-butyl (R)-((3-(6-chloro-3-(4,4-difluoroazepan-1-yl)-5-methylpyridazine-4-carboxamido)phenyl)(methyl)(oxo)-λ⁶-sulfaneylidene)carbamate (200 mg, 0.358 mmol), Zn(CN)₂ (42 mg, 0.358 mmol), Pd₂(dba)₃ (66 mg, 0.072 mmol), DPPF (79 mg, 0.143 mmol) and Zn (5 mg, 0.072 mmol) in DMA (2 mL) was heated at 120° C. for 16 hours under an atmosphere of N₂. After the reaction was completed, the mixture was cooled to room temperature. The resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtOAc=1/1) to give tert-butyl (R)-((3-(6-cyano-3-(4,4-difluoroazepan-1-yl)-5-methylpyridazine-4-carboxamido)phenyl)(methyl)(oxo)-λ⁶-sulfaneylidene)carbamate (150 mg, 76.4%) as a colorless oil. LCMS (ESI) calcd. for C₂₅H₃₁F₂N₆O₄S [M+H]⁺ m/z 549.21, found 549.20.

Step 2: (R)-6-cyano-3-(4,4-difluoroazepan-1-yl)-5-methyl-N-(3-(S-methylsulfonimidoyl)phenyl)pyridazine-4-carboxamide: A solution of tert-butyl (R)-((3-(6-cyano-3-(4,4-difluoroazepan-1-yl)-5-methylpyridazine-4-carboxamido)phenyl)(methyl)(oxo)-λ⁶-sulfaneylidene)carbamate (150 mg, 0.274 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (10 mL×3). The combined organic layers were washed with brine, dried over sodium sulfate, concentrated under vacuum. The residue was purified by prep-HPLC (Gemini 5 um C₁₈ column, 150*21.2 mm, eluting with 35% to 80% MeCN/H₂O containing 0.1% NH₃—H₂O) to provide (R)-6-cyano-3-(4,4-difluoroazepan-1-yl)-5-methyl-N-(3-(S-methylsulfonimidoyl)phenyl)pyridazine-4-carboxamide (27.4 mg, 22.3%) as a white solid. ¹H NMR (400 MHz, DMSO-d₆) δ 11.09 (s, 1H), 8.21 (s, 1H), 7.83 (s, 1H), 7.71-7.48 (m, 2H), 4.21 (s, 1H), 3.95-8.82 (m, 2H), 3.76-3.64 (m, 2H), 3.06 (s, 3H), 2.39-2.30 (m, 5H), 2.11-2.00 (m, 2H), 1.94-1.85 (m, 2H). LCMS (ESI) calcd. for C₂₀H₂₃F₂N₆O₂S [M+H]⁺ m/z 449.16, found 449.05.

### Example 140

### (R)-3-(4,4-difluoroazepan-1-yl)-5-methyl-N-(3-(S-methylsulfonimidoyl)phenyl)-6-(2-oxa-6-azaspiro[3.3]heptan-6-yl)pyridazine-4-carboxamide

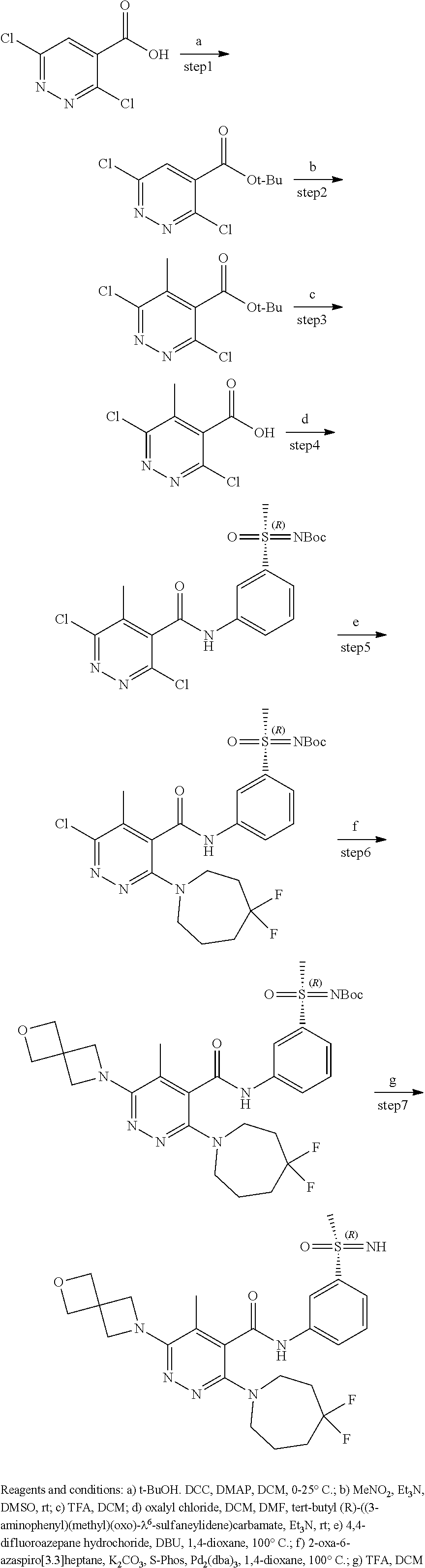

Step 1: Preparation of tert-butyl 3,6-dichloropyridazine-4-carboxylate: A mixture of 3,6-dichloropyridazine-4-carboxylic acid (20 g, 0.10 mol) in DCM (200 mL) was added dropwise t-BuOH (76.7 g, 10.4 mol) and DMAP (1.27 g, 0.01 mol) at 0° C. The mixture was stirred at 0° C. for 10 minutes, then DCC (42.8 g, 0.21 mol) was added dropwise. The resulting mixture was stirred at room temperature for 4 hours. Then the mixture was filtered through celite, and the filtrate was concentrated under vacuum. The residue was purified by flash column chromatography on silica gel (PE/EtOAc=10/1) to tert-butyl 3,6-dichloropyridazine-4-carboxylate (17 g, 68.5%) as a white solid. LCMS (ESI) calcd. for C₉H₁₁C₁₂N₂O₂ [M+H]⁺ m/z 249.02, found 249.00.

Step 2: tert-butyl 3,6-dichloro-5-methylpyridazine-4-carboxylate: A solution of tert-butyl 3,6-dichloropyridazine-4-carboxylate (15 g, 60.2 mmol) in DMSO (150 mL) was added dropwise MeNO₂ (18.4 g, 301 mmol) at room temperature. The mixture was stirred for 30 minutes and added dropwise TEA (9.14 g, 90.3 mmol) at 0° C. Then the mixture was stirred at room temperature for 16 hours. After the reaction was completed, the mixture was quenched with water (200 mL) and extracted with EtOAc (200 mL×3). The combined organic layers were washed with water and brine, dried over Na₂SO₄, filtered and concentrated under vacuum. The residue was purified by silica gel column chromatography (PE/EtOAc, 10/1) to afford tert-butyl 3,6-dichloro-5-methylpyridazine-4-carboxylate (10 g, 63.4%) as a yellow solid. LCMS (ESI) calcd. for C₁₀H₁₃C₁₂N₂O₂ [M+H]⁺ m/z 263.03, found 263.00.

Step 3: 3,6-dichloro-5-methylpyridazine-4-carboxylic acid: A solution of tert-butyl 3,6-dichloro-5-methylpyridazine-4-carboxylate (3 g, 11.4 mmol) in DCM (30 mL) was added TFA (30 mL) at room temperature. The reaction mixture was stirred at room temperature for 16 hours. The reaction was monitored by LCMS. After the reaction was completed, the mixture was concentrated to afford crude 3,6-dichloro-5-methylpyridazine-4-carboxylic acid (2 g, 85.6%) as yellow solid. LCMS (ESI) calcd. for C₆H₅C₂N₂O₂ [M+H]⁺ m/z 206.97, found 206.90.

Step 4: tert-butyl (R)-((3-(3,6-dichloro-5-methylpyridazine-4-carboxamido)phenyl)(methyl)(oxo)-λ⁶-sulfaneylidene)carbamate: To a solution of crude 3,6-dichloro-5-methylpyridazine-4-carboxylic acid (500 mg, 2.42 mmol) in DCM (5 mL) and DMF (10 uL) was added oxalyl chloride (460 mg, 3.62 mmol) at 0° C. The mixture was stirred at room temperature for 5 minutes. Then the solution was added dropwise to a solution of tert-butyl (R)-((3-aminophenyl)(methyl)(oxo)-λ⁶-sulfaneylidene)carbamate (653 mg, 2.42 mmol) and Et₃N (733 mg, 7.25 mmol). The mixture was stirred at room temperature for 1 hour. The resulting mixture was diluted with water (20 mL) and extracted with EtOAc (50 mL×3). The combined organic layers were washed with water and brine, dried over Na₂SO₄, filtered, concentrated to give the crude. The crude was purified by silica gel column chromatography (PE/EtOAc, 1/1) to afford tert-butyl (R)-((3-(3,6-dichloro-5-methylpyridazine-4-carboxamido)phenyl)(methyl)(oxo)-λ⁶-sulfaneylidene)carbamate (300 mg, 27%) as a yellow solid. LCMS (ESI) calcd. for C₁₈H₁₉Cl₂N₄O₄S [M−H]⁻ m/z 457.05, found 457.00.

Step 5: tert-butyl (R)-((3-(6-chloro-3-(4,4-difluoroazepan-1-yl)-5-methylpyridazine-4-carboxamido)phenyl)(methyl)(oxo)-λ⁶-sulfaneylidene)carbamate: A mixture tert-butyl (R)-((3-(3,6-dichloro-5-methylpyridazine-4-carboxamido)phenyl)(methyl)(oxo)-λ⁶-sulfaneylidene)carbamate (300 mg, 0.66 mmol) and 4,4-difluoroazepane hydrochloride (162.3 mg, 0.99 mmol) in 1,4-dioxane (6 mL) was added DBU (300 mg, 1.98 mmol) at room temperature. The reaction mixture was heated at 100° C. for 16 hours. Then the mixture was concentrated and directly purified by flash column chromatography on silica gel (PE/EtOAc=1/1) to give tert-butyl (R)-((3-(6-chloro-3-(4,4-difluoroazepan-1-yl)-5-methylpyridazine-4-carboxamido)phenyl)(methyl)(oxo)-λ⁶-sulfaneylidene)carbamate (200 mg, 55.4%). LCMS (ESI) calcd. C₂₄H₃₁ClF₂N₅O₄S [M+H]⁺ m/z 558.17, found 558.15.

Step 6: tert-butyl (R)-((3-(3-(4,4-difluoroazepan-1-yl)-5-methyl-6-(2-oxa-6-azaspiro[3.3]heptan-6-yl)pyridazine-4-carboxamido)phenyl)(methyl)(oxo)-λ⁶-sulfaneylidene)carbamate: A solution of tert-butyl (R)-((3-(6-chloro-3-(4,4-difluoroazepan-1-yl)-5-methylpyridazine-4-carboxamido)phenyl)(methyl)(oxo)-λ⁶sulfaneylidene)carbamate (100 mg, 0.18 mmol), 2-oxa-6-azaspiro[3.3]heptane (35.6 mg, 0.36 mmol), K₂CO₃(74.3 mg, 0.54 mmol), S-Phos (14.7 mg, 0.036 mmol) and Pd₂(dba)₃(16.4 mg, 0.018 mmol) in 1,4-dioxane (3 mL) was heated at 100° C. for overnight under an atmosphere of N₂. LCMS showed the reaction was completed. The mixture was filtered through celite, and the filtrate was concentrated under vacuum. The residue was purified by flash column chromatography on silica gel (PE/EtOAc=1/2) to give tert-butyl (R)-((3-(3-(4,4-difluoroazepan-1-yl)-5-methyl-6-(2-oxa-6-azaspiro[3.3]heptan-6-yl)pyridazine-4-carboxamido)phenyl)(methyl)(oxo)-λ⁶-sulfaneylidene)carbamate (50 mg, 49%) as a yellow oil. LCMS (ESI) calcd. for C₂₉H₃₉F₂N₆O₅S [M+H]⁺ m/z 621.27, found 621.25.

Step 7: (R)-3-(4,4-difluoroazepan-1-yl)-5-methyl-N-(3-(S-methylsulfonimidoyl)phenyl)-6-(2-oxa-6-azaspiro[3.3]heptan-6-yl)pyridazine-4-carboxamide: A solution of tert-butyl (R)-((3-(3-(4,4-difluoroazepan-1-yl)-5-methyl-6-(2-oxa-6-azaspiro[3.3]heptan-6-yl)pyridazine-4-carboxamido)phenyl)(methyl)(oxo)-λ⁶-sulfaneylidene)carbamate (50 mg, 0.08 mmol) in DCM (3 mL) was added TFA (1 mL) at room temperature. The reaction mixture was stirred at room temperature for 2 hours. After the reaction was completed, the mixture was diluted with water (10 mL) and extracted with DCM (10 mL×3). The combine organic phases were washed with brine, dried over sodium sulfate, concentrated under vacuum. The residue was purified by prep-HPLC (Gemini 5 um C₁₈ column, 150*21.2 mm, eluting with 35% to 80% ACN/H₂O containing 0.05% NH₃) to (R)-3-(4,4-difluoroazepan-1-yl)-5-methyl-N-(3-(S-methylsulfonimidoyl)phenyl)-6-(2-oxa-6-azaspiro[3.3]heptan-6-yl)pyridazine-4-carboxamide (9.5 mg, 22.8%) as a white solid. ¹H NMR (400 MHz, DMSO-d₆) δ 10.95 (s, 1H), 8.32 (s, 1H), 7.91 (d, J=8.9 Hz, 1H), 7.72 (d, J=8.0 Hz, 1H), 7.62 (t, J=7.9 Hz, 1H), 4.66 (s, 4H), 4.26 (s, 1H), 4.15 (s, 4H), 3.31-3.28 (m, 4H), 3.08 (s, 3H), 2.34-2.21 (m, 4H), 2.15 (s, 3H), 1.84 (d, J=5.2 Hz, 2H). LCMS (ESI) calcd. for C₂₄H₃₁F₂N₆O₃S [M+H]⁺ m/z 521.22, found 521.15.

### Example 141

### N-(3-carbamoylbicyclo[1.1.1]pentan-1-yl)-3-(4,4-difluoroazepan-1-yl)-5-methyl-6-(1-methyl-1H-pyrazol-4-yl)pyridazine-4-carboxamide

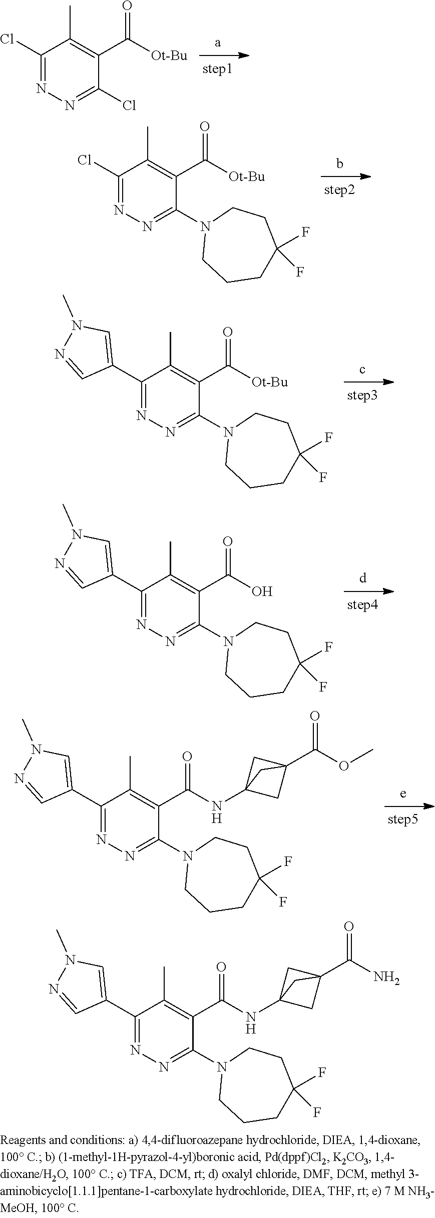

Step 1: tert-butyl 6-chloro-3-(4,4-difluoroazepan-1-yl)-5-methylpyridazine-4-carboxylate: A mixture of tert-butyl 3,6-dichloro-5-methylpyridazine-4-carboxylate (1 g, 3.9 mmol) and 4,4-difluoroazepane hydrochloride (1 g, 5.85 mmol) in 1,4-dioxane (15 mL) was added DIEA (2.52 g, 19.5 mmol) at room temperature. The reaction mixture was heated at 100° C. for 16 hours. Then the mixture was concentrated and directly purified by flash column chromatography on silica gel (PE/EtOAc=1/1) to give tert-butyl 6-chloro-3-(4,4-difluoroazepan-1-yl)-5-methylpyridazine-4-carboxylate (700 mg, 49.7%) as a white solid. LCMS (ESI) calcd. for C₁₆H₂₃ClF₂N₃O₂ [M+H]⁺ m/z 362.14, found 362.05.

Step 2: tert-butyl 3-(4,4-difluoroazepan-1-yl)-5-methyl-6-(1-methyl-1H-pyrazol-4-yl)pyridazine-4-carboxylate: A solution of tert-butyl 6-chloro-3-(4,4-difluoroazepan-1-yl)-5-methylpyridazine-4-carboxylate (700 mg, 1.93 mmol), (1-methyl-1H-pyrazol-4-yl)boronic acid (292.4 mg, 2.32 mmol), K₂CO₃ (1068 mg, 7.74 mmol) and Pd(dppf)Cl₂ (283 mg, 0.39 mmol) in 1,4-dioxane/H₂O (4/1, 15 mL) was heated at 100° C. for overnight under an atmosphere of N₂. LCMS showed the reaction was completed. The mixture was filtered through celite, and the filtrate was concentrated under vacuum. The residue was purified by flash column chromatography on silica gel (PE/EtOAc=2/1 to 1/2) to give tert-butyl 3-(4,4-difluoroazepan-1-yl)-5-methyl-6-(1-methyl-1H-pyrazol-4-yl)pyridazine-4-carboxylate (500 mg, 63.7%) as a yellow oil. LCMS (ESI) calcd. for C₂₀H₂₈F₂N₅O₂ [M+H]⁺ m/z 408.22, found 408.20.

Step 3: 3-(4,4-difluoroazepan-1-yl)-5-methyl-6-(1-methyl-1H-pyrazol-4-yl)pyridazine-4-carboxylic acid: To a solution of tert-butyl 3-(4,4-difluoroazepan-1-yl)-5-methyl-6-(1-methyl-1H-pyrazol-4-yl)pyridazine-4-carboxylate (500 mg, 1.229 mmol) in DCM (5 mL) was added TFA (5 mL). The mixture was stirred at room temperature for 3 hours. After the reaction was completed, the solution was quenched with saturated aqueous NaHCO₃ (30 mL) and extracted with DCM (30 mL×3). The combined organic phases were washed with brine, dried over sodium sulfate, and concentrated under vacuum to give the crude 3-(4,4-difluoroazepan-1-yl)-5-methyl-6-(1-methyl-1H-pyrazol-4-yl)pyridazine-4-carboxylic acid (300 mg) as a yellow solid. LCMS (ESI) calcd. for C₁₆H₂₀F₂N₅O₂ [M+H]⁺ m/z 352.16, found 352.15.

Step 4: methyl 3-(3-(4,4-difluoroazepan-1-yl)-5-methyl-6-(1-methyl-1H-pyrazol-4-yl)pyridazine-4-carboxamido)bicyclo[1.1.1]pentane-1-carboxylate: A solution of 3-(4,4-difluoroazepan-1-yl)-5-methyl-6-(1-methyl-1H-pyrazol-4-yl)pyridazine-4-carboxylic acid (150 mg, 0.426 mmol) in DCM (6 mL) and DMF (0.02 mL) was added oxalyl chloride (97 mg, 0.767 mmol). The solution was stirred at room temperature for 30 min. Then the solution was concentrated under vacuum. The residue was diluted with THF (5 mL) and added to a stirred solution of methyl 3-aminobicyclo[1.1.1]pentane-1-carboxylate hydrochloride (123 mg, 0.639 mmol) and DIEA (165 mg, 1.278 mmol) in THE (5 mL). The mixture was stirred at room temperature for 3 hours. Then the mixture was quenched with water (20 mL) and extracted with DCM (20 mL×3). The combined organic phases were washed with brine, dried over sodium sulfate, concentrated under vacuum. The residue was purified by flash column chromatography on silica gel (PE/EtOAc=1/1 to 1/3) to give methyl 3-(3-(4,4-difluoroazepan-1-yl)-5-methyl-6-(1-methyl-1H-pyrazol-4-yl)pyridazine-4-carboxamido)bicyclo[1.1.1]pentane-1-carboxylate 7 (100 mg, 49.50%) as a yellow solid. LCMS (ESI) calcd. for C₂₃H₂₉F₂N₆O₃ [M+H]⁺ m/z 475.23, found 475.15.

Step 5: N-(3-carbamoylbicyclo[1.1.1]pentan-1-yl)-3-(4,4-difluoroazepan-1-yl)-5-methyl-6-(1-methyl-1H-pyrazol-4-yl)pyridazine-4-carboxamide: A solution of methyl 3-(3-(4,4-difluoroazepan-1-yl)-5-methyl-6-(1-methyl-1H-pyrazol-4-yl)pyridazine-4-carboxamido)bicyclo[1.1.1]pentane-1-carboxylate (100 mg, 0.211 mmol) in 7M NH₃-MeOH (5 mL) was heated at 100° C. for 8 hours in a sealed tube. Then the mixture was concentrated under vacuum. The residue was purified by prep-HPLC (Gemini 5 um C₁₈ column, 150*21.2 mm, eluting with 35% to 80% MeCN/H₂O containing 0.1% formic acid) to provide N-(3-carbamoylbicyclo[1.1.1]pentan-1-yl)-3-(4,4-difluoroazepan-1-yl)-5-methyl-6-(1-methyl-1H-pyrazol-4-yl)pyridazine-4-carboxamide (33 mg, 34.38% yield) as a white solid. ¹H NMR (400 MHz, DMSO-d₆, ppm) δ 9.28 (s, 1H), 8.13 (s, 1H), 7.85 (s, 1H), 7.30 (s, 1H), 7.01 (s, 1H), 3.91 (s, 3H), 3.72-3.65 (m, 2H), 3.61 (t, J=6.1 Hz, 2H), 2.37-2.27 (m, 2H), 2.23 (s, 3H), 2.21 (s, 6H), 2.12-2.01 (m, 2H), 1.92-1.82 (m, 2H). LCMS (ESI) calcd. for C₂₂H₂₈F₂N₇O₂ [M+H]⁺ m/z 460.23, found 460.10.

### Example 142

### 3-(2-(4,4-difluoroazepan-1-yl)-4-methyl-5-(1-methyl-1H-pyrazol-4-yl)nicotinamido)bicyclo[1.1.1]pentane-1-carboxylic acid

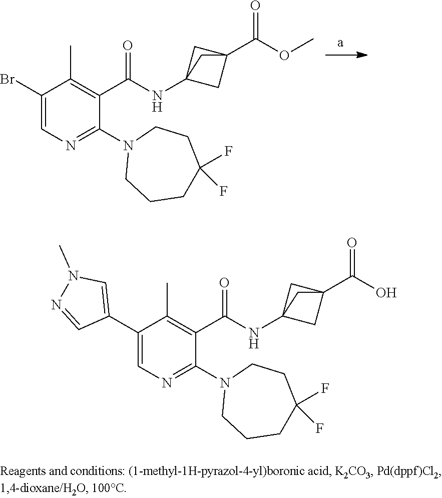 [SMILES: COC(=O)C12CC(NC(=O)c3c(N4CCCC(F)(F)CC4)ncc(Br)c3C)(C1)C2 ; Cc1c(-c2cnn(C)c2)cnc(N2CCCC(F)(F)CC2)c1C(=O)NC12CC(C(=O)O)(C1)C2]

3-(2-(4,4-difluoroazepan-1-yl)-4-methyl-5-(1-methyl-1H-pyrazol-4-yl)nicotinamido)bicyclo[1.1.1]pentane-1-carboxylic acid: A mixture of methyl 3-(5-bromo-2-(4,4-difluoroazepan-1-yl)-4-methylnicotinamido)bicyclo[1.1.1]pentane-1-carboxylate (30 mg, 0.064 mmol), (1-methyl-1H-pyrazol-4-yl)boronic acid (12 mg, 0.095 mmol), K₂CO₃ (26 mg, 0.191 mmol) and Pd(dppf)Cl₂ (5 mg, 0.006 mmol) in 1,4-dioxane/H₂O (4/1, 10 mL) was heated at 100° C. for 16 h under an atmosphere of N₂. After the reaction was completed, the mixture was cooled to room temperature. The resulting solution was diluted with water (30 mL) and extracted with EtOAc (30 mL×3). The combined organic phases were washed with brine, dried over Na₂SO₄, concentrated under vacuum. The residue was purified by prep-HPLC (Gemini 5 um C₁₈ column, 150*21.2 mm, eluting with 35% to 80% MeCN/H₂O containing 0.1% formic acid) to provide 3-(2-(4,4-difluoroazepan-1-yl)-4-methyl-5-(1-methyl-1H-pyrazol-4-yl)nicotinamido)bicyclo[1.1.1]pentane-1-carboxylic acid (12 mg, 41.38%) as a white solid. ¹H NMR (400 MHz, DMSO-d₆) δ 12.41 (s, 1H), 9.08 (s, 1H), 8.07 (s, 1H), 7.85 (s, 1H), 7.56 (s, 1H), 3.87 (s, 3H), 3.59-3.51 (m, 4H), 2.25 (s, 8H), 2.17 (s, 3H), 2.09-1.98 (m, 2H), 1.88-1.80 (m, 2H). LCMS (ESI) calcd. for C₂₃H₂₈F₂N₅O₃ [M+H]⁺ m/z 460.21, found 460.10.

### Example 143

### (S)-4-(4,4-difluoroazepan-1-yl)-6-methyl-N-(3-(S-methylsulfonimidoyl)phenyl)-2-(trifluoromethyl)pyrimidine-5-carboxamide

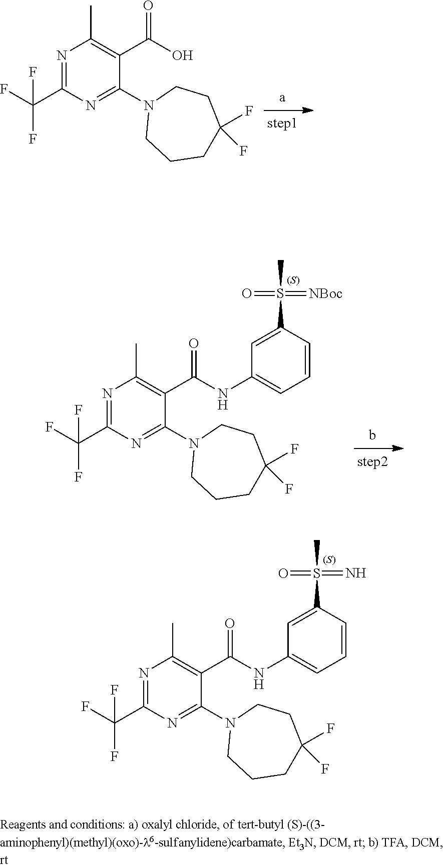

Step 1: tert-butyl (S)-((3-(4-(4,4-difluoroazepan-1-yl)-6-methyl-2-(trifluoromethyl)pyrimidine-5-carboxamido)phenyl)(methyl)(oxo)-λ⁶-sulfaneylidene)carbamate: A solution of 4-(4,4-difluoroazepan-1-yl)-6-methyl-2-(trifluoromethyl)pyrimidine-5-carboxylic acid (100 mg, 0.295 mmol) in DCM (3 mL) and DMF (one drop) was added oxalyl chloride (127 mg, 1 mmol). The solution was stirred at room temperature for 5 min. Then the solution was added to a stirred solution of tert-butyl (S)-((3-aminophenyl)(methyl)(oxo)-λ⁶-sulfaneylidene)carbamate (159 mg, 0.59 mmol) and TEA (141 mg, 1.39 mmol) in DCM (3 mL) at 0° C. The resulting mixture was stirred at 25° C. for 1 hour. Then the mixture was quenched with water (10 mL) and extracted with DCM (10 mL×2). The combined organic layers were washed with brine, dried over sodium sulfate, concentrated under vacuum. The residue was purified by flash column chromatography on silica gel (PE/EtOAc=1/1) to provide tert-butyl (S)-((3-(4-(4,4-difluoroazepan-1-yl)-6-methyl-2-(trifluoromethyl)pyrimidine-5-carboxamido)phenyl)(methyl)(oxo)-λ⁶-sulfaneylidene)carbamate (60 mg, 34.4% yield) as a brown oil. LCMS (ESI) calcd. for C₂₅H₃₁F₅N₅O₄S [M+H]⁺ m/z 592.20, found 592.10.

Step 2: (S)-4-(4,4-difluoroazepan-1-yl)-6-methyl-N-(3-(S-methylsulfonimidoyl)phenyl)-2-(trifluoromethyl)pyrimidine-5-carboxamide: A solution of tert-butyl (S)-(3-(4-(4,4-difluoroazepan-1-yl)-6-methyl-2-(trifluoromethyl)pyrimidine-5-carboxamido)phenyl)(methyl)(oxo)-λ⁶-sulfaneylidene)carbamate (60 mg, 0.101 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (10 mL×3). The combined organic layers were washed with brine, dried over sodium sulfate, concentrated under vacuum. The residue was purified by prep-HPLC (Gemini 5 um C₁₈ column, 150*21.2 mm, eluting with 35% to 80% MeCN/H₂O containing 0.1% NH₃·H₂O) to provide (S)-4-(4,4-difluoroazepan-1-yl)-6-methyl-N-(3-(S-methylsulfonimidoyl)phenyl)-2-(trifluoromethyl)pyrimidine-5-carboxamide (31.5 mg, 63.3%) as a white solid. ¹H NMR (400 MHz, DMSO-d₆) δ 11.00 (s, 1H), 8.28 (s, 1H), 7.88 (d, J=7.9 Hz, 1H), 7.65 (dt, J=15.8, 7.9 Hz, 2H), 4.25 (s, 1H), 3.83-3.54 (m, 4H), 3.07 (s, 3H), 2.41 (s, 3H), 2.35-2.24 (m, 2H), 2.12-1.99 (m, 2H), 1.90-1.78 (m, 2H). LCMS (ESI) calcd. for C₂₀H₂₃F₅N₅O₂S [M+H]⁺ m/z 492.15, found 492.10.

### Example 144

### (R)-2-(4,4-difluoroazepan-1-yl)-4-methyl-N-(3-(S-methylsulfonimidoyl)phenyl)-5-(1H-pyrazol-4-yl)nicotinamide

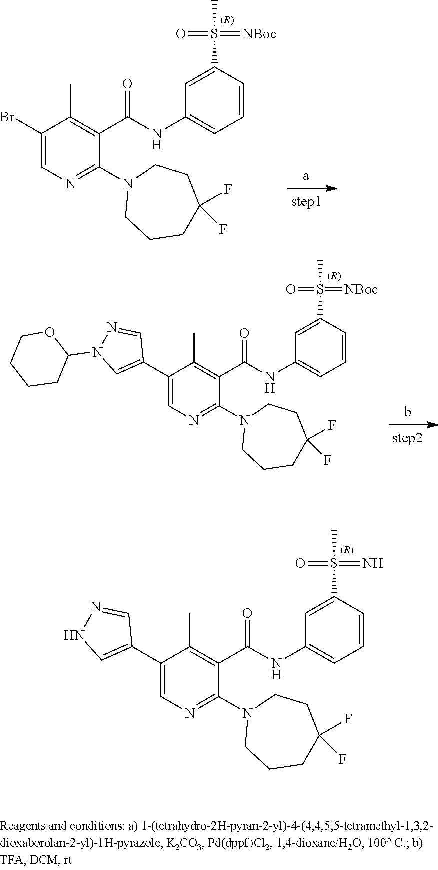

Step 1: tert-butyl ((1R)-(3-(2-(4,4-difluoroazepan-1-yl)-4-methyl-5-(1-(tetrahydro-2H-pyran-2-yl)-1H-pyrazol-4-yl)nicotinamido)phenyl)(methyl)(oxo)-λ⁶-sulfaneylidene)carbamate: A mixture of tert-butyl (R)-((3-(5-bromo-2-(4,4-difluoroazepan-1-yl)-4-methylnicotinamido)phenyl)(methyl)(oxo)-λ⁶-sulfaneylidene)carbamate (150 mg, 0.25 mmol), 1-(tetrahydro-2H-pyran-2-yl)-4-(4,4,5,5-tetramethyl-1,3,2-dioxaborolan-2-yl)-1H-pyrazole (70 mg, 0.50 mmol), K₂CO₃ (69 mg, 0.50 mmol) and Pd(dppf)Cl₂ (18 mg, 0.02 mmol) in dioxane (8 mL) and H₂O (2 mL) was heated at 100° C. for 16 hours under an atmosphere of N₂. After the reaction was completed, the mixture was cooled to room temperature. The resulting solution was diluted with water (30 mL) and extracted with EtOAc (30 mL×3). The combined organic phases were washed with brine, dried over Na₂SO₄, concentrated under vacuum. The residue was purified by flash column chromatography on silica gel (PE/EtOAc=1/2) to give tert-butyl ((1R)-(3-(2-(4,4-difluoroazepan-1-yl)-4-methyl-5-(1-(tetrahydro-2H-pyran-2-yl)-1H-pyrazol-4-yl)nicotinamido)phenyl)(methyl)(oxo)-λ⁶-sulfaneylidene)carbamate (110 mg, 65.3%) as a yellow solid. LCMS (ESI) calcd. for C₃₃H₄₃F₂N₆O₅S [M+H]⁺ m/z 673.30, found 673.25.

Step 2: (R)-2-(4,4-difluoroazepan-1-yl)-4-methyl-N-(3-(S-methylsulfonimidoyl)phenyl)-5-(1H-pyrazol-4-yl)nicotinamide: A solution of tert-butyl ((1R)-(3-(2-(4,4-difluoroazepan-1-yl)-4-methyl-5-(1-(tetrahydro-2H-pyran-2-yl)-1H-pyrazol-4-yl)nicotinamido)phenyl)(methyl)(oxo)-λ⁶-sulfaneylidene)carbamate (110 mg, 0.16 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 30% to 90% MeCN/H₂O containing 0.1% NH₃) to provide (R)-2-(4,4-difluoroazepan-1-yl)-4-methyl-N-(3-(S-methylsulfonimidoyl)phenyl)-5-(1H-pyrazol-4-yl)nicotinamide (41.4 mg, 52.9% yield) as a white solid. ¹H NMR (400 MHz, DMSO-d₆) δ 12.97 (s, 1H), 10.85 (s, 1H), 8.37 (t, J=1.8 Hz, 1H), 8.19 (s, 1H), 7.90 (d, J=8.1 Hz, 1H), 7.80 (s, 2H), 7.66 (d, J=8.0 Hz, 1H), 7.58 (t, J=7.9 Hz, 1H), 4.22 (s, 1H), 3.63-3.50 (m, 4H), 3.06 (s, 3H), 2.31-2.18 (m, 5H), 2.09-1.90 (m, 2H), 1.87-1.71 (m, 2H). LCMS (ESI) calcd. for C₂₃H₂₇F₂N₆O₂S [M+H]⁺ m/z 489.19, found 489.10.

### Example 145

### (S)-5-bromo-2-(4,4-difluoroazepan-1-yl)-4-methyl-N-(3-(S-methylsulfonimidoyl)phenyl)nicotinamide

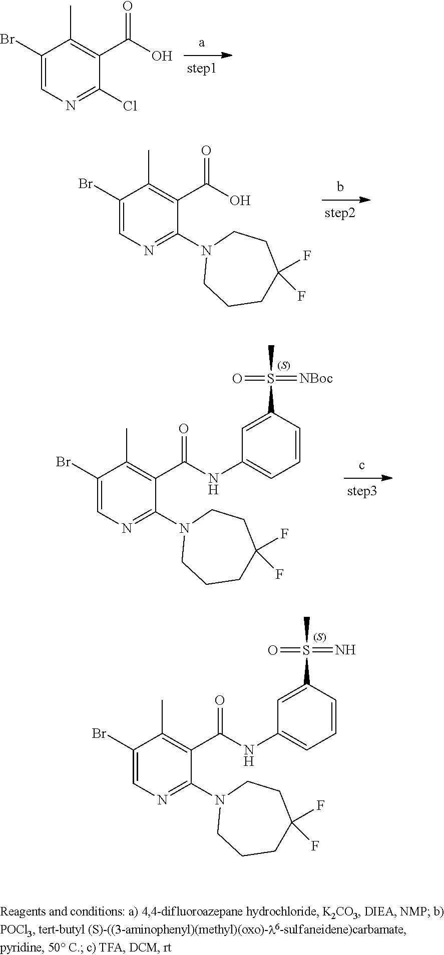

Step 1: 5-bromo-2-(4,4-difluoroazepan-1-yl)-4-methylnicotinic acid: A solution of 5-bromo-2-chloro-4-methylnicotinic acid (4.00 g, 15.9 mmol), 4,4-difluoroazepane hydrochloride (5.43 g, 31.8 mmol), K₂CO₃ (13.20 g, 95.4 mmol) and DIEA (8.21 g, 63.6 mmol) in NMP (120 mL) was heated at 140° C. for 6 hours. LCMS showed the reaction was completed. The mixture was diluted with water. Then pH of the resulting mixture was adjusted to 4 with 2N aqueous HCl. The solution was extracted with EtOAc. The combined organic layers were dried over anhydrous Na₂SO₄ and concentrated to afford the crude. The crude was purified by flash column chromatography on silica gel (PE/EtOAc=2/1) to provide 5-bromo-2-(4,4-difluoroazepan-1-yl)-4-methylnicotinic acid (1.6 g, 28.6%) as a yellow solid. LCMS (ESI) calcd. for C₁₃H₁₆BrF₂N₂O₂ [M+H]⁺ m/z 351.04, found 348.90.

Step 2: tert-butyl (S)-((3-(5-bromo-2-(4,4-difluoroazepan-1-yl)-4-methylnicotinamido)phenyl)(methyl)(oxo)-λ⁶-sulfaneylidene)carbamate: A mixture of 5-bromo-2-(4,4-difluoroazepan-1-yl)-4-methylnicotinic acid (1.60 g, 4.58 mmol) and tert-butyl (S)-((3-aminophenyl)(methyl)(oxo)-λ⁶-sulfaneylidene)carbamate (2.48 g, 9.17 mmol) in pyridine (30 mL) was added POCl₃ (500 L) dropwise at 50° C. The reaction solution was stirred at 50° C. for 2 hours. After the reaction was completed, the resulting solution was diluted with water (200 mL) and extracted with EtOAc (80 mL×3). The combined organic layers were washed with brine, dried over sodium sulfate, concentrated under vacuum. The residue was purified by flash column chromatography on silica gel (PE/EtOAc=2/1) to give tert-butyl (S)-((3-(5-bromo-2-(4,4-difluoroazepan-1-yl)-4-methylnicotinamido)phenyl)(methyl)(oxo)-λ⁶-sulfaneylidene)carbamate (1.55 g, 56.4%) as a yellow solid. LCMS (ESI) calcd. for C₂₅H₃₂BrF₂N₄O₄S [M+H]⁺ m/z 603.13, found 601.05.

Step 3: (S)-5-bromo-2-(4,4-difluoroazepan-1-yl)-4-methyl-N-(3-(S-methylsulfonimidoyl)phenyl)nicotinamide: A solution of tert-butyl (S)-((3-(5-bromo-2-(4,4-difluoroazepan-1-yl)-4-methylnicotinamido)phenyl)(methyl)(oxo)-λ⁶-sulfaneylidene)carbamate (100 mg, 0.17 mmol) in DCM (5 mL) was added TFA (0.5 mL) at room temperature. The reaction mixture was stirred at room temperature for 2 hours. After the reaction was completed, the solution was diluted with water (10 mL) and extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 50% to 95% MeCN/H₂O containing 0.1% NH₃) to give (S)-5-bromo-2-(4,4-difluoroazepan-1-yl)-4-methyl-N-(3-(S-methylsulfonimidoyl)phenyl)nicotinamide (30.6 mg, 38.2%) as a white solid. ¹H NMR (400 MHz, DMSO-d₆) δ 10.89 (s, 1H), 8.37-8.24 (m, 2H), 7.89 (d, J=9.0 Hz, 1H), 7.68 (d, J=8.3 Hz, 1H), 7.59 (t, J=7.9 Hz, 1H), 4.23 (s, 1H), 3.63-3.48 (m, 4H), 3.06 (s, 3H), 2.31-2.18 (m, 5H), 2.06-1.92 (m, 2H), 1.84-1.74 (m, 2H). LCMS (ESI) calcd. for C₂₀H₂₄BrF₂N₄O₂S [M+H]⁺ m/z 501.08, found 500.95.

### Example 146

### (R)-2-(4,4-difluoroazepan-1-yl)-5-(1,3-dimethyl-1H-pyrazol-4-yl)-4-methyl-N-(3-(S-methylsulfonimidoyl)phenyl)nicotinamide

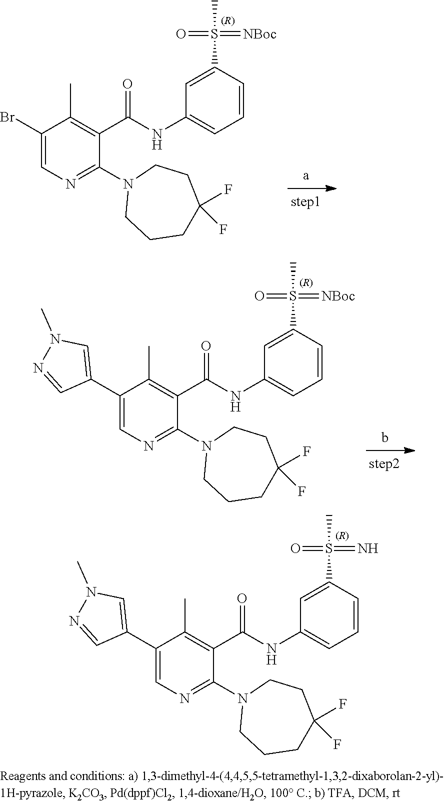

Step 1: tert-butyl (R)-((3-(2-(4,4-difluoroazepan-1-yl)-5-(1,3-dimethyl-1H-pyrazol-4-yl)-4-methylnicotinamido)phenyl)(methyl)(oxo)-λ⁶-sulfaneylidene)carbamate: A mixture of tert-butyl (R)-((3-(5-bromo-2-(4,4-difluoroazepan-1-yl)-4-methylnicotinamido)phenyl)(methyl)(oxo)-λ⁶-sulfaneylidene)carbamate (100 mg, 0.17 mmol), 1,3-dimethyl-4-(4,4,5,5-tetramethyl-1,3,2-dioxaborolan-2-yl)-1H-pyrazole (73.3 mg, 0.33 mmol), K₂CO₃ (68.9 mg, 0.5 mmol) and Pd(dppf)Cl₂ (24.3 mg, 0.03 mmol) in 1,4-dioxane/H₂O (4/1, 5 mL) was heated at 100° C. for 5 hours under an atmosphere of N₂. After the reaction was completed, the mixture was cooled to room temperature. The resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtOAc=1/1) to give tert-butyl (R)-((3-(2-(4,4-difluoroazepan-1-yl)-5-(1,3-dimethyl-1H-pyrazol-4-yl)-4-methylnicotinamido)phenyl)(methyl)(oxo)-λ⁶-sulfaneylidene)carbamate (82 mg, 78.3%) as a yellow oil. LCMS (ESI) calcd. for C₃₀H₃₉F₂N₆O₄S [M+H]⁺ m/z 617.27, found 617.20.

Step 2: (R)-2-(4,4-difluoroazepan-1-yl)-5-(1,3-dimethyl-1H-pyrazol-4-yl)-4-methyl-N-(3-(S-methylsulfonimidoyl)phenyl)nicotinamide: A solution of give tert-butyl (R)-((3-(2-(4,4-difluoroazepan-1-yl)-5-(1,3-dimethyl-1H-pyrazol-4-yl)-4-methylnicotinamido)phenyl)(methyl)(oxo)-λ⁶-sulfaneylidene)carbamate (82 mg, 0.133 mmol) in DCM (5 mL) was added TFA (1 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 50% to 85% MeCN/H₂O containing 0.05% NH₃) to provide (R)-2-(4,4-difluoroazepan-1-yl)-5-(1,3-dimethyl-1H-pyrazol-4-yl)-4-methyl-N-(3-(S-methylsulfonimidoyl)phenyl)nicotinamide (45.2 mg, 65.9%) as a white solid. ¹H NMR (400 MHz, DMSO-d₆) δ 10.85 (s, 1H), 8.37 (s, 1H), 7.99 (s, 1H), 7.90 (d, J=8.0 Hz, 1H), 7.66 (d, J=7.9 Hz, 1H), 7.63 (s, 1H), 7.58 (t, J=7.9 Hz, 1H), 4.23 (s, 1H), 3.80 (s, 3H), 3.64-3.53 (m, 4H), 3.06 (s, 3H), 2.29-2.23 (m, 2H), 2.10 (s, 3H), 2.06 (s, 3H), 2.03-1.94 (m, 2H), 1.84-1.75 (m, 2H). LCMS (ESI) calcd. for C₂₅H₃₁F₂N₆O₂S [M+H]⁺ m/z 517.22, found 517.15

### Example 147 (R)-2-(4,4-difluoroazepan-1-yl)-4-methyl-N-(3-(S-methylsulfonimidoyl)phenyl)-5-(oxazol-5-yl)nicotinamide

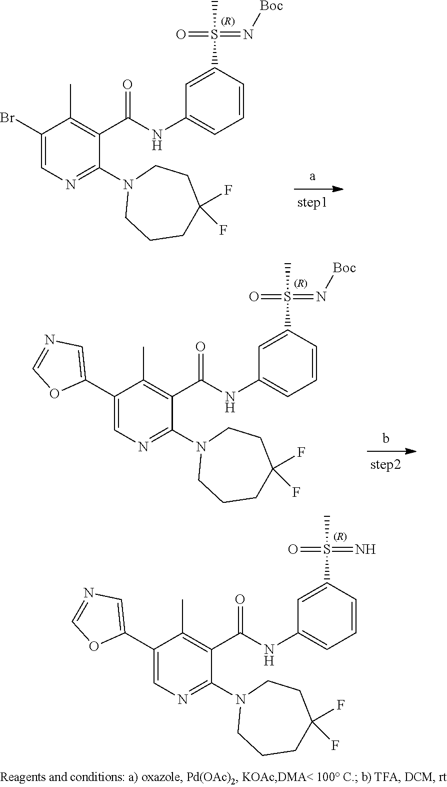

Step 1: tert-butyl (R)-((3-(2-(4,4-difluoroazepan-1-yl)-4-methyl-5-(oxazol-5-yl)nicotinamido)phenyl)(methyl)(oxo)-λ⁶-sulfaneylidene)carbamate: A mixture of tert-butyl (R)-((3-(5-bromo-2-(4,4-difluoroazepan-1-yl)-4-methylnicotinamido)phenyl)(methyl)(oxo)-λ⁶-sulfaneylidene)carbamate(100 mg, 0.17 mmol), oxazole (23 mg, 0.33 mmol), KOAc (50 mg, 0.51 mmol) and Pd(OAc)₂ (6 mg, 0.03 mmol) in DMA (5 mL) was heated at 100° C. for 16 hours under an atmosphere of N₂. After the reaction was completed, the mixture was cooled to room temperature. The resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtOAc=1/5) to give tert-butyl (R)-((3-(2-(4,4-difluoroazepan-1-yl)-4-methyl-5-(oxazol-5-yl)nicotinamido)phenyl)(methyl)(oxo)-λ⁶-sulfaneylidene)carbamate (65 mg, 65.31% yield) as a yellow oil. LCMS (ESI) calcd. for C₂₈H₃₄F₂N₅O₅S [M+H]⁺ m/z 590.23, found 590.15.

Step 2: (R)-2-(4,4-difluoroazepan-1-yl)-4-methyl-N-(3-(S-methylsulfonimidoyl)phenyl)-5-(oxazol-5-yl)nicotinamide: A solution of tert-butyl (R)-((3-(2-(4,4-difluoroazepan-1-yl)-4-methyl-5-(oxazol-5-yl)nicotinamido)phenyl)(methyl)(oxo)-λ⁶-sulfaneylidene)carbamate (65 mg, 0.11 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.1% formic acid) to provide (R)-2-(4,4-difluoroazepan-1-yl)-4-methyl-N-(3-(S-methylsulfonimidoyl)phenyl)-5-(oxazol-5-yl)nicotinamide (11.2 mg, 20.74%) as a white solid. ¹H NMR (400 MHz, DMSO-d₆) δ 10.93 (s, 1H), 8.48 (s, 1H), 8.40 (s, 1H), 8.35 (s, 1H), 7.91 (d, J=7.7 Hz, 1H), 7.68 (d, J=7.7 Hz, 1H), 7.60 (t, J=7.9 Hz, 1H), 7.40 (s, 1H), 4.24 (s, 1H), 3.72-3.65 (m, 2H), 3.61 (t, J=5.9 Hz, 2H), 3.06 (s, 3H), 2.35-2.21 (m, 5H), 2.08-1.94 (m, 2H), 1.88-1.77 (m, 2H). LCMS (ESI) calcd. for C₂₃H₂₆F₂N₅O₃S [M+H]⁺ m/z 490.17, found 490.15.

### Example 148

### (R)-6-cyano-2-(4,4-difluoroazepan-1-yl)-4-methyl-N-(3-(S-methylsulfonimidoyl)phenyl)nicotinamide

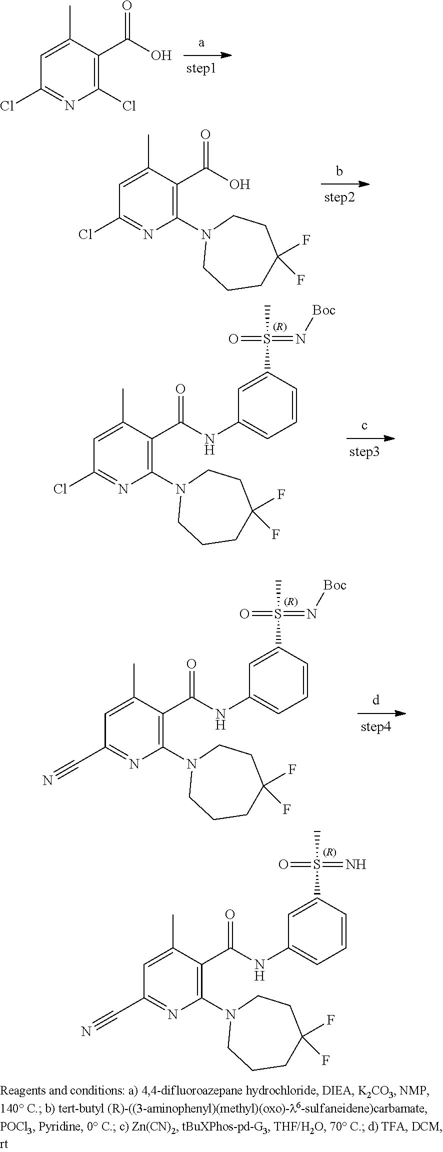

Step 1: 6-chloro-2-(4,4-difluoroazepan-1-yl)-4-methylnicotinic acid: A mixture of 2,6-dichloro-4-methylnicotinic acid (1 g, 4.95 mmol) and 4,4-difluoroazepane hydrochloride (916 mg, 5.34 mmol) in NMP (15 mL) was added DIEA (1.88 g, 14.56 mmol) and K₂CO₃ (2.01 g, 14.56 mmol) at room temperature. The reaction mixture was heated at 140° C. for 16 hours. After the reaction was completed, the solution was diluting with DCM (100 mL) and washed with brine (100 mL×3), dried over sodium sulfate, then concentrated and purified by flash column chromatography on silica gel (PE/EtOAc=1/1 to 1/3) to give 6-chloro-2-(4,4-difluoroazepan-1-yl)-4-methylnicotinic acid (1.1 g, 66.9%) as a white solid. LCMS (ESI) calcd. for C₁₃H₁₆ClF₂N₂O₂ [M+H]⁺ m/z 305.09, found 304.95.

Step 2: tert-butyl (R)-((3-(6-chloro-2-(4,4-difluoroazepan-1-yl)-4-methylnicotinamido)phenyl)(methyl)(oxo)-λ⁶-sulfaneylidene)carbamate: A mixture of 6-chloro-2-(4,4-difluoroazepan-1-yl)-4-methylnicotinic acid (700 mg, 2.30 mmol) and tert-butyl (R)-((3-aminophenyl)(methyl)(oxo)-λ⁶-sulfaneylidene)carbamate (748 mg, 2.76 mmol) in pyridine (6 mL) was added POCl₃ (100 L) dropwise at 0° C. The reaction solution was stirred at 0° C. for 2 hours. After the reaction was completed, the resulting solution was diluted with water (50 mL) and extracted with EtOAc (50 mL×3). The combined organic layers were washed with brine, dried over sodium sulfate, concentrated under vacuum. The residue was purified by flash column chromatography on silica gel (PE/EtOAc=2/1) to give tert-butyl (R)-((3-(6-chloro-2-(4,4-difluoroazepan-1-yl)-4-methylnicotinamido)phenyl)(methyl)(oxo)-λ⁶-sulfaneylidene)carbamate (500 mg, 39.0%) as a yellow solid. LCMS (ESI) calcd. for C₂₅H₃₂ClF₂N₄O₄S [M+H]⁺ m/z 557.18, found 557.20

Step 3: tert-butyl (R)-((3-(6-cyano-2-(4,4-difluoroazepan-1-yl)-4-methylnicotinamido)phenyl)(methyl)(oxo)-λ⁶-sulfaneylidene)carbamate: A solution of tert-butyl (R)-((3-(6-chloro-2-(4,4-difluoroazepan-1-yl)-4-methylnicotinamido)phenyl)(methyl)(oxo)-λ⁶-sulfaneylidene)carbamate (300 mg, 0.54 mmol), Zn(CN)₂ (126 mg, 1.08 mmol) and tBuXphos-pd-G₃ (87 mg, 0.11 mmol) in THF/H₂O (4/1, 5 mL) was heated at 70° C. overnight under an atmosphere of N₂. LCMS showed the reaction was completed. The mixture was filtered through celite, and the filtrate was concentrated under vacuum. The residue was purified by flash column chromatography on silica gel (PE/EtOAc=2/1 to 1/2) to give tert-butyl (R)-((3-(6-cyano-2-(4,4-difluoroazepan-1-yl)-4-methylnicotinamido)phenyl)(methyl)(oxo)-λ⁶-sulfaneylidene)carbamate (200 mg, 67.8%) as a yellow oil. LCMS (ESI) calcd. for C₂₆H₃₂F₂N₅O₄S [M+H]⁺ m/z 548.22, found 548.20

Step 4: (R)-6-cyano-2-(4,4-difluoroazepan-1-yl)-4-methyl-N-(3-(S-methylsulfonimidoyl)phenyl)nicotinamide: A solution of tert-butyl (R)-((3-(6-cyano-2-(4,4-difluoroazepan-1-yl)-4-methylnicotinamido)phenyl)(methyl)(oxo)-λ⁶-sulfaneylidene)carbamate (100 mg, 0.18 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (5 mL×3). The combined organic phases were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.1% NH₃) to give (R)-6-cyano-2-(4,4-difluoroazepan-1-yl)-4-methyl-N-(3-(S-methylsulfonimidoyl)phenyl)nicotinamide (30.6 mg, 37.4%) as a white solid. ¹H NMR (400 MHz, DMSO-d₆) δ 10.92 (s, 1H), 8.29 (t, J=2.0 Hz, 1H), 7.89 (dd, J=8.1, 2.2 Hz, 1H), 7.72-7.66 (m, 1H), 7.60 (t, J=7.9 Hz, 1H), 7.33 (s, 1H), 4.24 (s, 1H), 3.68-3.59 (m, 2H), 3.56 (t, J=6.1 Hz, 2H), 3.06 (s, 3H), 2.34-2.22 (m, 5H), 2.08-1.96 (m, 2H), 1.86-1.75 (m, 2H). LCMS (ESI) calcd. for C₂₁H₂₄F₂N₅O₂S [M+H]⁺ m/z 448.16, found 448.05.

### Example 149

### (R)-2-(4,4-difluoroazepan-1-yl)-5-(3,3-difluoroazetidin-1-yl)-4-methyl-N-(3-(S-methylsulfonimidoyl)phenyl)nicotinamide

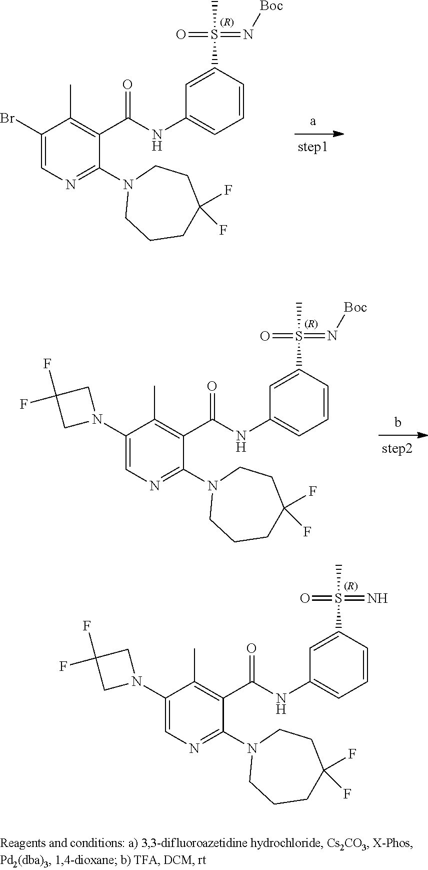

Step 1: tert-butyl (R)-((3-(2-(4,4-difluoroazepan-1-yl)-5-(3,3-difluoroazetidin-1-yl)-4-methylnicotinamido)phenyl)(methyl)(oxo)-λ⁶-sulfaneylidene)carbamate: A mixture of tert-butyl (R)-((3-(5-bromo-2-(4,4-difluoroazepan-1-yl)-4-methylnicotinamido)phenyl)(methyl)(oxo)-λ⁶-sulfaneylidene)carbamate (150 mg, 0.250 mmol) and 3,3-difluoroazetidine hydrochloride (220 mg, 1.25 mmol) in 1,4-dioxane (10 mL) was added Cs₂CO₃ (540 mg, 1.25 mmol), Pd₂(dba)₃ (40 mg, 0.025 mmol) and X-Phos (40 mg, 0.05 mmol). The mixture was heated at 100° C. for 6 hours under an atmosphere of N₂. The reaction was monitored by LCMS. After the reaction was completed, the mixture was cooled to room temperature, diluted with water (20 mL) and extracted with DCM (20 mL×3). The combined organic layers were washed with brine, dried over Na₂SO₄, and concentrated under vacuum. The residue was purified by flash column chromatography on silica gel (PE/EtOAc=2/1 to 1/1) to give tert-butyl (R)-((3-(2-(4,4-difluoroazepan-1-yl)-5-(3,3-difluoroazetidin-1-yl)-4-methylnicotinamido)phenyl)(methyl)(oxo)-λ⁶-sulfaneylidene)carbamate (77 mg, 49.00%) as a yellow oil. LCMS (ESI) calcd. for C₂₈H₃₆F₄N₅O₄S [M+H]⁺ m/z 614.24, found 614.15.

Step 2: (R)-2-(4,4-difluoroazepan-1-yl)-5-(3,3-difluoroazetidin-1-yl)-4-methyl-N-(3-(S-methylsulfonimidoyl)phenyl)nicotinamide: A solution of tert-butyl (R)-((3-(2-(4,4-difluoroazepan-1-yl)-5-(3,3-difluoroazetidin-1-yl)-4-methylnicotinamido)phenyl)(methyl)(oxo)-λ⁶-sulfaneylidene)carbamate (77 mg, 0.125 mmol) in DCM (5 mL) was added TFA (0.5 mL) at room temperature. The reaction mixture was stirred at room temperature for 2 hours. After the reaction was completed, the solution was diluted with water (10 mL) and extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.1% formic acid) to obtain (R)-2-(4,4-difluoroazepan-1-yl)-5-(3,3-difluoroazetidin-1-yl)-4-methyl-N-(3-(S-methylsulfonimidoyl)phenyl)nicotinamide (32.8 mg, 50.1%) as a white solid. ¹H NMR (400 MHz, DMSO-d₆) δ 10.75 (s, 1H), 8.35-8.34 (m, 1H), 7.90 (d, J=8.8 Hz, 1H), 7.72 (s, 1H), 7.65 (d, J=8.4 Hz, 1H), 7.57 (t, J=8 Hz, 1H), 4.28 (t, J=12.4 Hz, 4H), 4.21 (s, 1H), 3.46-3.43 (m, 2H), 3.41-3.38 (m, 2H), 3.04 (s, 3H), 2.22-2.16 (m, 2H), 2.08 (s, 3H), 1.98-1.92 (m, 2H), 1.76-1.72 (m, 2H). LCMS (ESI) calcd. for

### Example 150

### (R)-4-(4,4-difluoroazepan-1-yl)-6-methyl-2-(1-methyl-1H-pyrazol-4-yl)-N-(3-(S-methylsulfonimidoyl)phenyl)pyrimidine-5-carboxamide

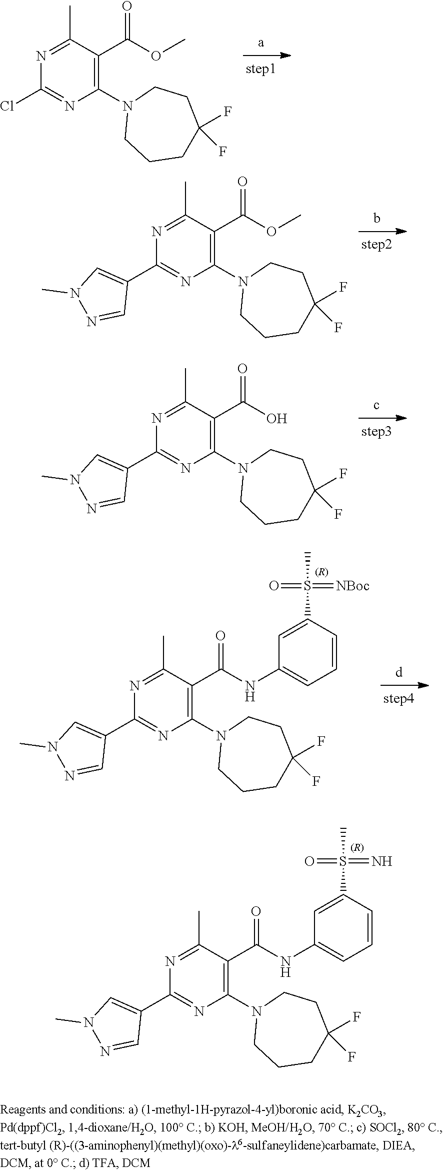

Step 1: methyl 4-(4,4-difluoroazepan-1-yl)-6-methyl-2-(1-methyl-1H-pyrazol-4-yl)pyrimidine-5-carboxylate: A mixture of methyl 2-chloro-4-(4,4-difluoroazepan-1-yl)-6-methylpyrimidine-5-carboxylate (300 mg, 0.94 mmol), (1-methyl-1H-pyrazol-4-yl)boronic acid (178 mg, 1.41 mmol), K₂CO₃ (259 mg, 1.88 mmol), Pd(dppf)Cl₂ (66 mg, 0.09 mmol) in 1,4-dioxane/H₂O (4/1, 10 mL) was heated at 100° C. for 16 h under an atmosphere of N₂. After the reaction was completed, the mixture was cooled to room temperature. The resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtOAc=3/1) to give methyl 4-(4,4-difluoroazepan-1-yl)-6-methyl-2-(1-methyl-1H-pyrazol-4-yl)pyrimidine-5-carboxylate (280 mg, 81.6% yield) as a yellow solid. LCMS (ESI) calcd. for C₁₇H₂₁F₂N₅O₂Na [M+Na]⁺ m/z 366.17, found 366.10.

Step 2: 4-(4,4-difluoroazepan-1-yl)-6-methyl-2-(1-methyl-1H-pyrazol-4-yl)pyrimidine-5-carboxylic acid: To a solution of methyl 4-(4,4-difluoroazepan-1-yl)-6-methyl-2-(1-methyl-1H-pyrazol-4-yl)pyrimidine-5-carboxylate (280 mg, 0.76 mmol) in MeOH/H₂O (1/1, 10 mL) was added KOH (428 mg, 7.65 mmol) at room temperature. The mixture was heated at 70° C. for 16 hours. After the reaction was completed, the mixture was concentrated to remove most MeOH. The aqueous phase was adjusted to pH=3-4 with 1N HCl then extracted with EtOAc (20 mL×3). The combined organic phases were washed with brine, dried with Na₂SO₄, and concentrated under reduced pressure to afford 4-(4,4-difluoroazepan-1-yl)-6-methyl-2-(1-methyl-1H-pyrazol-4-yl)pyrimidine-5-carboxylic acid (190 mg, 71.0%) as a yellow solid. LCMS (ESI) calcd. for C₁₆H₂₀F₂N₅O₂ [M+H]⁺ m/z 352.16, found 352.15.

Step 3: tert-butyl (R)-((3-(4-(4,4-difluoroazepan-1-yl)-6-methyl-2-(1-methyl-1H-pyrazol-4-yl)pyrimidine-5-carboxamido)phenyl)(methyl)(oxo)-λ⁶-sulfaneylidene)carbamate: A solution of 4-(4,4-difluoroazepan-1-yl)-6-methyl-2-(1-methyl-1H-pyrazol-4-yl)pyrimidine-5-carboxylic acid (100 mg, 0.28 mmol) in SOCl₂ (2 mL) was heated to 80° C. and stirred for 1 hour. Then the solution was concentrated under vacuum to provide the chloride intermediate. The chloride intermediate was added to a stirred solution of tert-butyl (R)-((3-aminophenyl)(methyl)(oxo)-λ⁶-sulfaneylidene)carbamate (91 mg, 0.34 mmol) and DIEA (72 mg, 0.56 mmol) in DCM (2 mL) at 0° C. The resulting mixture was stirred at 25° C. for 1 hour. Then the mixture was quenched with water (10 mL) and extracted with DCM (10 mL×2). The combined organic layers were washed with brine, dried over sodium sulfate, concentrated under vacuum. The residue was purified by flash column chromatography on silica gel (PE/EtOAc=1/1) to afford tert-butyl (R)-((3-(4-(4,4-difluoroazepan-1-yl)-6-methyl-2-(1-methyl-1H-pyrazol-4-yl)pyrimidine-5-carboxamido)phenyl)(methyl)(oxo)-λ⁶-sulfaneylidene)carbamate (65 mg, 38.4%) as a yellow solid. LCMS (ESI) calcd. for C₂₈H₃₆F₂N₇O₄S [M+H]⁺ m/z 604.25, found 604.25.

Step 4: (R)-4-(4,4-difluoroazepan-1-yl)-6-methyl-2-(1-methyl-1H-pyrazol-4-yl)-N-(3-(S-methylsulfonimidoyl)phenyl)pyrimidine-5-carboxamide: A solution of tert-butyl (R)-((3-(4-(4,4-difluoroazepan-1-yl)-6-methyl-2-(1-methyl-1H-pyrazol-4-yl)pyrimidine-5-carboxamido)phenyl)(methyl)(oxo)-λ⁶-sulfaneylidene)carbamate (65 mg, 0.11 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 30% to 90% MeCN/H₂O containing 0.05% NH₃) to provide (R)-4-(4,4-difluoroazepan-1-yl)-6-methyl-2-(1-methyl-1H-pyrazol-4-yl)-N-(3-(S-methylsulfonimidoyl)phenyl)pyrimidine-5-carboxamide (20.8 mg, 37.5%) as a white solid. ¹H NMR (400 MHz, DMSO-d₆) δ 10.86 (s, 1H), 8.31 (s, 2H), 7.97 (s, 1H), 7.88 (d, J=8.1 Hz, 1H), 7.67 (d, J=7.9 Hz, 1H), 7.59 (t, J=7.9 Hz, 1H), 4.23 (s, 1H), 3.90 (s, 3H), 3.77 (s, 2H), 3.62 (t, J=6.0 Hz, 2H), 3.06 (s, 3H), 2.41-2.25 (m, 5H), 2.11-1.95 (m, 2H), 1.89-1.79 (m, 2H). LCMS (ESI) calcd. for C₂₃H₂₈F₂N₇O₂S [M+H]⁺ m/z 504.20, found 504.15.

### Example 151

### (R)-2-(4,4-difluoroazepan-1-yl)-4-methyl-6-(1-methyl-1H-pyrazol-4-yl)-N-(3-(S-methylsulfonimidoyl)phenyl)nicotinamide

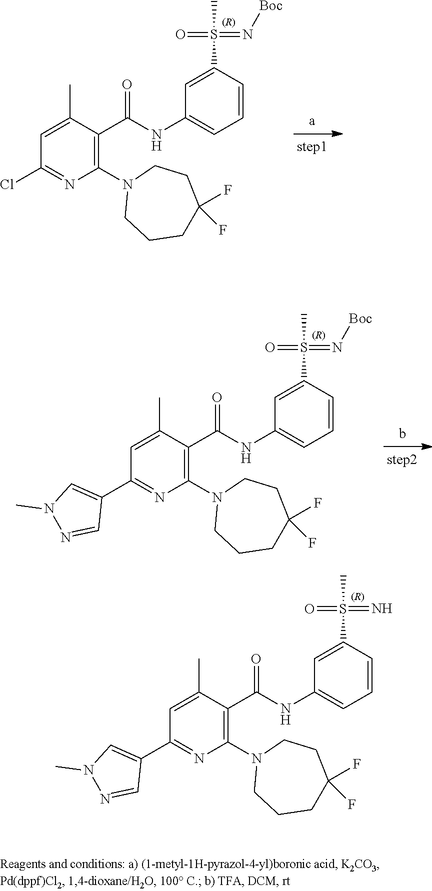

Step 1: tert-butyl (R)-((3-(2-(4,4-difluoroazepan-1-yl)-4-methyl-6-(1-methyl-1H-pyrazol-4-yl)nicotinamido)phenyl)(methyl)(oxo)-λ⁶-sulfaneylidene)carbamate: A solution of tert-butyl (R)-((3-(6-chloro-2-(4,4-difluoroazepan-1-yl)-4-methylnicotinamido) phenyl)(methyl)(oxo)-λ⁶-sulfaneylidene)carbamate (150 mg, 0.27 mmol), (1-methyl-1H-pyrazol-4-yl)boronic acid (40 mg, 0.32 mmol), K₂CO₃ (74 mg, 0.54 mmol) and Pd(dppf)Cl₂ (20 mg, 0.03 mmol) in 1,4-dioxane (10 mL) and H₂O (2.5 mL) was heated at 100° C. for 6 hours. LCMS showed the reaction was completed. The mixture was diluted with water (10 mL) and extracted with EtOAc (10 mL×3). The combined organic layers were washed with brine, dried over sodium sulfate, concentrated under vacuum. The residue was purified by flash column chromatography on silica gel (PE/EtOAc=1/10) to provide tert-butyl (R)-((3-(2-(4,4-difluoroazepan-1-yl)-4-methyl-6-(1-methyl-1H-pyrazol-4-yl)nicotinamido)phenyl)(methyl)(oxo)-λ⁶-sulfaneylidene)carbamate (120 mg, 74.07%) as yellow oil. LCMS (ESI) calcd. for C₂₉H₃₇F₂N₆O₄S [M+H]⁺ m/z 603.26, found 603.25.

Step 2: (R)-2-(4,4-difluoroazepan-1-yl)-4-methyl-6-(1-methyl-1H-pyrazol-4-yl)-N-(3-(S-methylsulfonimidoyl)phenyl)nicotinamide: A solution of tert-butyl (R)-((3-(2-(4,4-difluoroazepan-1-yl)-4-methyl-6-(1-methyl-1H-pyrazol-4-yl)nicotinamido)phenyl)(methyl)(oxo)-λ⁶-sulfaneylidene)carbamate (120 mg, 0.20 mmol) in DCM (5 mL) was added TFA (0.5 mL) at room temperature. The reaction mixture was stirred at room temperature for 1 hour. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 20% to 95% MeCN/H₂O containing 0.05% NH₃·H₂O) to provide (R)-2-(4,4-difluoroazepan-1-yl)-4-methyl-6-(1-methyl-1H-pyrazol-4-yl)-N-(3-(S-methylsulfonimidoyl)phenyl)nicotinamide (53.25 mg, 52.26% yield) as a white solid. ¹H NMR (400 MHz, DMSO-d₆) δ 10.71 (s, 1H), 8.37 (t, J=1.8 Hz, 1H), 8.21 (s, 1H), 7.96 (s, 1H), 7.90 (d, J=8.0 Hz, 1H), 7.65 (d, J=7.9 Hz, 1H), 7.57 (t, J=7.9 Hz, 1H), 6.97 (s, 1H), 4.40 (bs, 1H), 3.89 (s, 3H), 3.67-3.65 (m, 2H), 3.55 (t, J=6.1 Hz, 2H), 3.07 (s, 3H), 2.33-2.34 (m, 2H), 2.25 (s, 3H), 2.00-1.94 (m, 2H), 1.84-1.81 (m, 2H). LCMS (ESI) calcd. for C₂₄H₂₉F₂N₆O₂S [M+H]⁺ m/z 503.21, found 503.15.

### Example 152

### (R)-2-(4,4-difluoroazepan-1-yl)-5-(3,5-dimethyl-1H-pyrazol-4-yl)-4-methyl-N-(3-(S-methylsulfonimidoyl)phenyl)nicotinamide

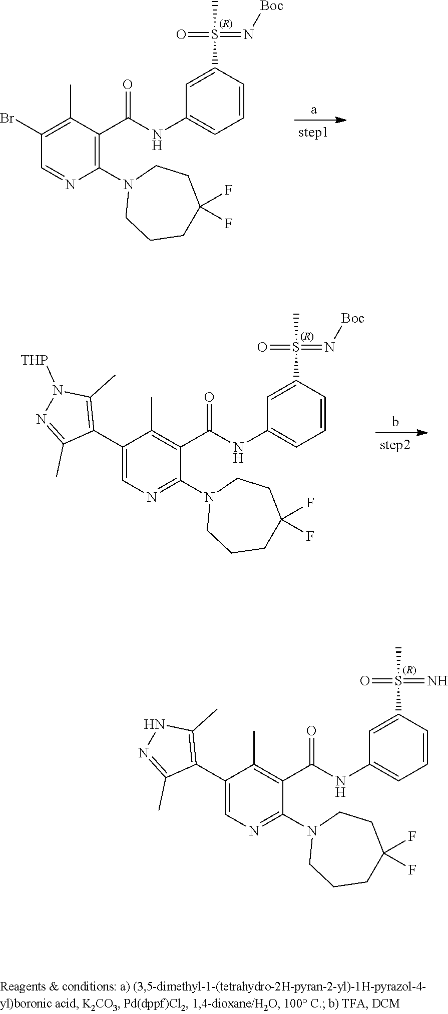

Step 1: tert-butyl ((1R)-(3-(2-(4,4-difluoroazepan-1-yl)-5-(3,5-dimethyl-1-(tetrahydro-2H-pyran-2-yl)-1H-pyrazol-4-yl)-4-methylnicotinamido)phenyl)(methyl)(oxo)-λ⁶-sulfaneylidene)carbamate: A mixture of tert-butyl (R)-((3-(5-bromo-2-(4,4-difluoroazepan-1-yl)-4-methylnicotinamido) phenyl)(methyl)(oxo)-λ⁶-sulfaneylidene)carbamate (100 mg, 0.17 mmol), (3,5-dimethyl-1-(tetrahydro-2H-pyran-2-yl)-1H-pyrazol-4-yl)boronic acid (112 mg, 0.50 mmol), K₂CO₃ (69 mg, 0.50 mmol), Pd(dppf)Cl₂ (22 mg, 0.02 mmol) in dioxane/H₂O (4/1, 10 mL) was heated at 100° C. for 16 hours under an atmosphere of N₂. After the reaction was completed, the mixture was cooled to room temperature. The resulting solution was diluted with water (50 mL) and extracted with EtOAc (40 mL×3). The combined organic phases were washed with brine, dried over Na₂SO₄, concentrated under vacuum. The residue was purified by flash column chromatography on silica gel (PE/EtOAc=3/1) to give tert-butyl ((1R)-(3-(2-(4,4-difluoroazepan-1-yl)-5-(3,5-dimethyl-1-(tetrahydro-2H-pyran-2-yl)-1H-pyrazol-4-yl)-4-methylnicotinamido)phenyl)(methyl)(oxo)-λ⁶-sulfaneylidene)carbamate (98 mg, 57.1%) as a yellow solid. LCMS (ESI) calcd. for C₃₅H₄₇F₂N₆O₅S [M+H]⁺ m/z 701.33, found 701.20.

Step 2: (R)-2-(4,4-difluoroazepan-1-yl)-5-(3,5-dimethyl-1H-pyrazol-4-yl)-4-methyl-N-(3-(S-methylsulfonimidoyl)phenyl)nicotinamide: A solution of tert-butyl ((1R)-(3-(2-(4,4-difluoroazepan-1-yl)-5-(3,5-dimethyl-1-(tetrahydro-2H-pyran-2-yl)-1H-pyrazol-4-yl)-4-methylnicotinamido)phenyl)(methyl)(oxo)-λ⁶-sulfaneylidene)carbamate (98 mg, 0.14 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (30 mL×3). The combined organic layers were washed with brine, dried over sodium sulfate, concentrated under vacuum. The residue was purified by prep-HPLC (Gemini 5 um C₁₈ column, 150*21.2 mm, eluting with 30% to 80% MeCN/H₂O containing 0.05% NH₃) to provide (R)-2-(4,4-difluoroazepan-1-yl)-5-(3,5-dimethyl-1H-pyrazol-4-yl)-4-methyl-N-(3-(S-methylsulfonimidoyl)phenyl)nicotinamide (41.5 mg, 57.3%) as a white solid. ¹H NMR (400 MHz, DMSO-d₆) δ 10.84 (s, 1H), 8.36 (s, 1H), 7.93 (d, J=5.7 Hz, 2H), 7.67-7.56 (m, 2H), 4.22 (s, 1H), 3.65-3.53 (m, 4H), 3.05 (s, 3H), 2.32-2.18 (m, 2H), 2.09-1.91 (m, 11H), 1.87-1.72 (m, 2H). LCMS (ESI) calcd. for C₂₅H₃₁F₂N₆O₂S [M+H]⁺ m/z 517.22, found 517.15.

### Example 153

### (R)-6-(4,4-difluoroazepan-1-yl)-4-methyl-N₅-(3-(S-methylsulfonimidoyl)phenyl)pyridine-2,5-dicarboxamide

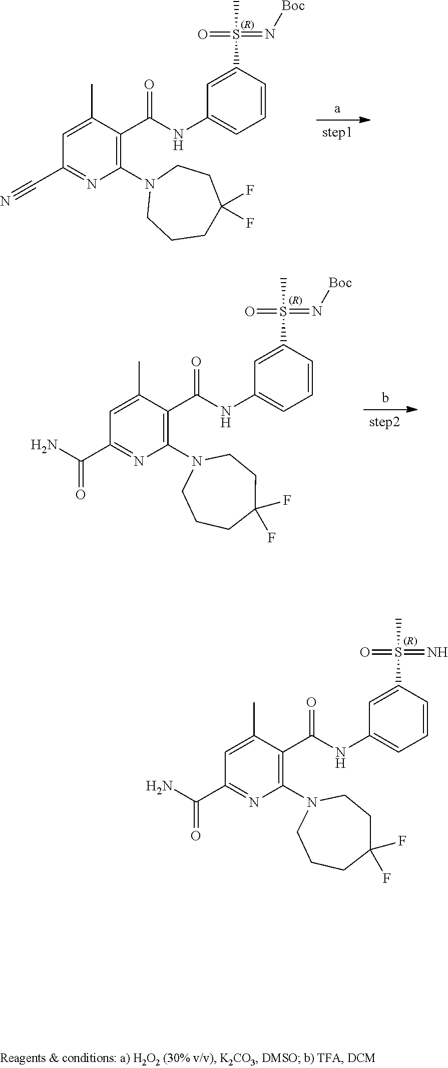

Step 1: tert-butyl (R)-((3-(6-carbamoyl-2-(4,4-difluoroazepan-1-yl)-4-methylnicotinamido)phenyl)(methyl)(oxo)-λ⁶-sulfaneylidene)carbamate: A mixture of tert-butyl (R)-((3-(6-cyano-2-(4,4-difluoroazepan-1-yl)-4-methylnicotinamido) phenyl)(methyl)(oxo)-λ⁶-sulfaneylidene)carbamate (100 mg, 0.18 mmol), K₂CO₃ (76 mg, 0.55 mmol) and 30% H₂O₂(2 mL) in DMSO (5 mL) was stirred at room temperature for 4 hour. After the reaction was completed, the mixture was cooled to room temperature. The resulting solution was diluted with water (30 mL) and extracted with EtOAc (30 mL×3). The combined organic phases were washed with brine, dried over Na₂SO₄, concentrated under vacuum. The residue was purified by flash column chromatography on silica gel (PE/EtOAc=1/5) to give tert-butyl (R)-((3-(6-carbamoyl-2-(4,4-difluoroazepan-1-yl)-4-methylnicotinamido)phenyl)(methyl)(oxo)-λ⁶-sulfaneylidene)carbamate (72 mg, 69.51%) as a yellow oil. LCMS (ESI) calcd. for C₂₆H₃₄F₂N₅O₅S [M+H]⁺ m/z 566.23, found 566.20.

Step 2: (R)-6-(4,4-difluoroazepan-1-yl)-4-methyl-N₅-(3-(S-methylsulfonimidoyl)phenyl)pyridine-2,5-dicarboxamide: A solution of tert-butyl (R)-((3-(6-carbamoyl-2-(4,4-difluoroazepan-1-yl)-4-methylnicotinamido)phenyl)(methyl)(oxo)-λ⁶-sulfaneylidene)carbamate (72 mg, 0.13 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 45% to 95% MeCN/H₂O containing 0.1% formic acid) to provide (R)-6-(4,4-difluoroazepan-1-yl)-4-methyl-N₅-(3-(S-methylsulfonimidoyl)phenyl)pyridine-2,5-dicarboxamide (26.1 mg, 43.93%) as a white solid. ¹H NMR (400 MHz, DMSO-d₆) δ 10.84 (s, 1H), 8.33 (d, J=1.7 Hz, 1H), 7.93-7.79 (m, 2H), 7.69-7.55 (m, 3H), 7.31 (s, 1H), 4.23 (s, 1H), 3.76-3.66 (m, 2H), 3.59 (t, J=6.0 Hz, 2H), 3.06 (s, 3H), 2.35-2.20 (s, 5H), 2.06-1.92 (m, 2H), 1.86-1.74 (m, 2H). LCMS (ESI) calcd. for C₂₁H₂₆F₂N₅O₃S [M+H]⁺ m/z 466.17, found 466.05.

### Example 154

### (R)-6-(4-cyanophenyl)-3-(4,4-difluoroazepan-1-yl)-5-methyl-N-(3-(S-methylsulfonimidoyl)phenyl)pyridazine-4-carboxamide

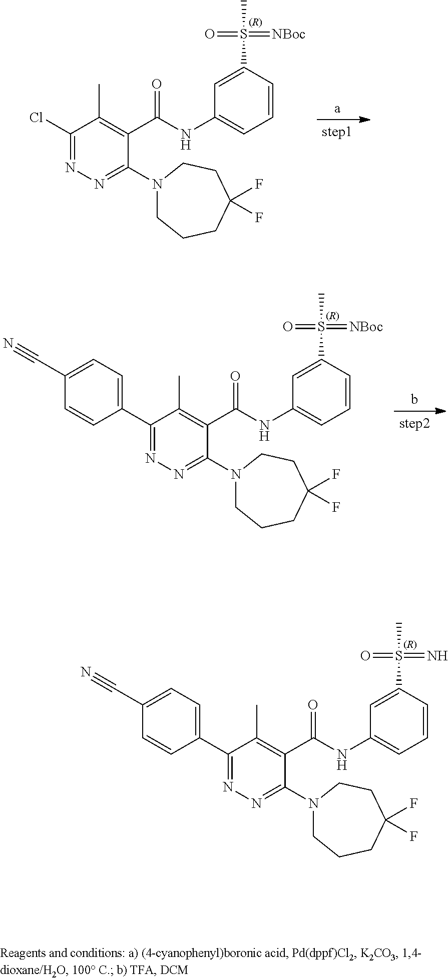

Step 1: tert-butyl (R)-((3-(6-(4-cyanophenyl)-3-(4,4-difluoroazepan-1-yl)-5-methylpyridazine-4-carboxamido)phenyl)(methyl)(oxo)-λ⁶-sulfaneylidene)carbamate: A mixture of tert-butyl (R)-((3-(6-chloro-3-(4,4-difluoroazepan-1-yl)-5-methylpyridazine-4-carboxamido)phenyl)(methyl)(oxo)-λ⁶-sulfaneylidene)carbamate (150 mg, 0.27 mmol), (4-cyanophenyl)boronic acid (79 mg, 0.54 mmol), K₂CO₃ (112 mg, 0.81 mmol) and Pd(dppf)Cl₂ (18 mg, 0.02 mmol) in dioxane (8 mL) and H₂O (2 mL) was heated at 100° C. for 16 hours under an atmosphere of N₂. After the reaction was completed, the mixture was cooled to room temperature. The resulting solution was diluted with water (40 mL) and extracted with EtOAc (40 mL×3). The combined organic phases were washed with brine, dried over Na₂SO₄, concentrated under vacuum. The residue was purified by flash column chromatography on silica gel (PE/EtOAc=1/1) to give tert-butyl (R)-((3-(6-(4-cyanophenyl)-3-(4,4-difluoroazepan-1-yl)-5-methylpyridazine-4-carboxamido)phenyl)(methyl)(oxo)-λ⁶-sulfaneylidene)carbamate (98 mg, 58.3%) as a yellow solid. LCMS (ESI) calcd. for C₃₁H₃₅F₂N₆O₄S [M+H]⁺ m/z 625.24, found 625.15.

Step 2: (R)-6-(4-cyanophenyl)-3-(4,4-difluoroazepan-1-yl)-5-methyl-N-(3-(S-methylsulfonimidoyl)phenyl)pyridazine-4-carboxamide: A solution of tert-butyl (R)-((3-(6-(4-cyanophenyl)-3-(4,4-difluoroazepan-1-yl)-5-methylpyridazine-4-carboxamido)phenyl)(methyl)(oxo)-λ⁶-sulfaneylidene)carbamate (98 mg, 0.16 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (30 mL×3). The combined organic phases were washed with brine, dried over sodium sulfate, concentrated under vacuum. The residue was purified by prep-HPLC (Gemini 5 um C₁₈ column, 150*21.2 mm, eluting with 35% to 90% MeCN/H₂O containing 0.1% NH₃) to provide (R)-6-(4-cyanophenyl)-3-(4,4-difluoroazepan-1-yl)-5-methyl-N-(3-(S-methylsulfonimidoyl)phenyl)pyridazine-4-carboxamide (21.3 mg, 25.9%) as a white solid. ¹H NMR (400 MHz, DMSO-d₆) δ 11.09 (s, 1H), 8.31 (s, 1H), 7.99 (d, J=8.4 Hz, 2H), 7.89 (d, J=7.9 Hz, 1H), 7.80 (d, J=8.4 Hz, 2H), 7.71 (d, J=7.6 Hz, 1H), 7.61 (t, J=7.9 Hz, 1H), 4.25 (s, 1H), 3.87-3.78 (m, 2H), 3.70-3.61 (m, 2H), 3.07 (s, 3H), 2.43-2.27 (m, 2H), 2.21 (s, 3H), 2.11-1.97 (m, 2H), 1.91-1.83 (m, 2H). LCMS (ESI) calcd. for C₂₆H₂₇F₂N₆O₂S [M+H]⁺ m/z 525.19, found 525.15.

### Example 155

### (R)-4-(4,4-difluoroazepan-1-yl)-6-methyl-N-(3-(S-methylsulfonimidoyl)phenyl)-2-morpholinopyrimidine-5-carboxamide

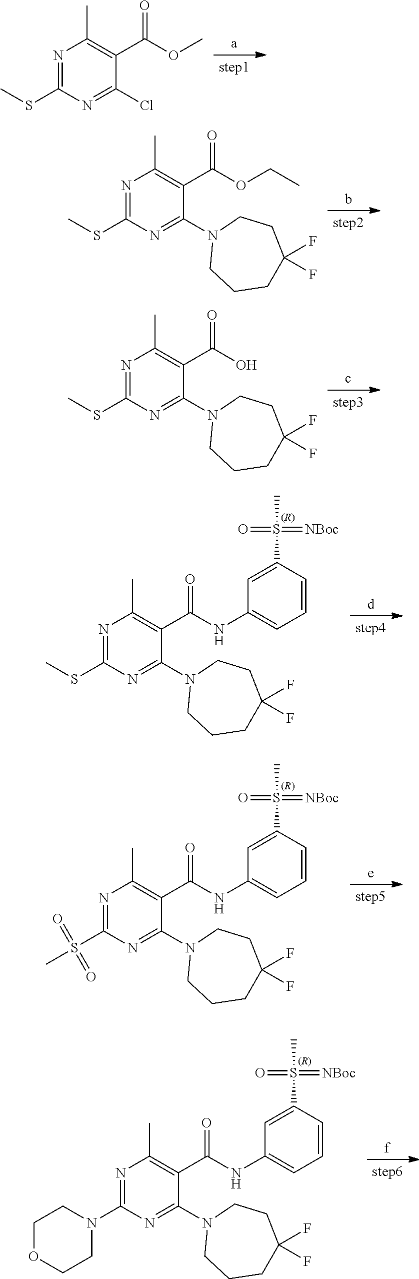

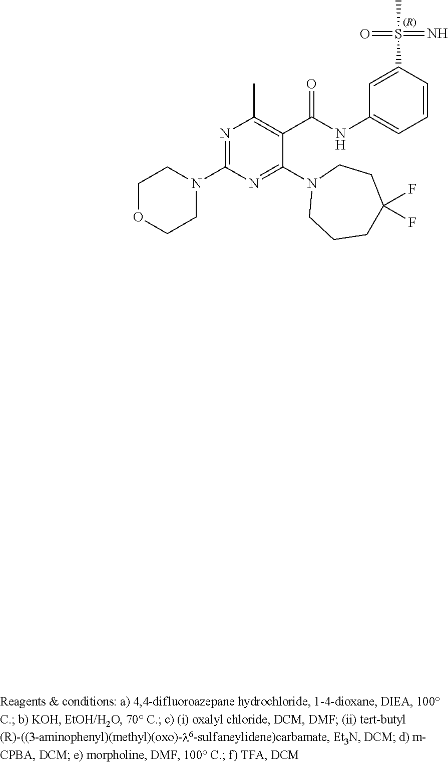

Step 1: ethyl 4-(4,4-difluoroazepan-1-yl)-6-methyl-2-(methylthio)pyrimidine-5-carboxylate: A mixture of ethyl 4-chloro-6-methyl-2-(methylthio)pyrimidine-5-carboxylate (1 g, 4.06 mmol) and 4,4-difluoroazepane hydrochloride (834 mg, 4.87 mmol) in 1,4-dioxane (20 mL) was added DIEA (1.05 g, 8.12 mmol) at room temperature. The reaction mixture was heated at 100° C. for 4 hours. Then the mixture was concentrated and directly purified by flash column chromatography on silica gel (PE/EtOAc=3/1) to give ethyl 4-(4,4-difluoroazepan-1-yl)-6-methyl-2-(methylthio)pyrimidine-5-carboxylate (1.3 g, 92.5%) as a yellow solid. LCMS (ESI) calcd. for C₁₅H₂₂F₂N₃O₂S [M+H]⁺ m/z 346.14, found 345.95.

Step 2: 4-(4,4-difluoroazepan-1-yl)-6-methyl-2-(methylthio)pyrimidine-5-carboxylic acid: To a solution of ethyl 4-(4,4-difluoroazepan-1-yl)-6-methyl-2-(methylthio)pyrimidine-5-carboxylate (1.3 g, 3.75 mmol) in EtOH/H₂O (1/1, 20 mL) was added KOH (421 mg, 7.50 mmol) at room temperature. The mixture was heated at 70° C. for 16 hours. After the reaction was completed, the mixture was concentrated to remove most EtOH. The aqueous phase was adjusted to pH=3-4 with 1N HCl then extracted with EtOAc (40 mL×3). The combined organic phases were washed with brine, dried with Na₂SO₄, and concentrated under reduced pressure to afford 4-(4,4-difluoroazepan-1-yl)-6-methyl-2-(methylthio)pyrimidine-5-carboxylic acid (860 mg, 72.1%) as a yellow solid. LCMS (ESI) calcd. for C₁₃H₁₈F₂N₃O₂S [M+H]⁺ m/z 318.11, found 317.95.

Step 3: tert-butyl (R)-((3-(4-(4,4-difluoroazepan-1-yl)-6-methyl-2-(methylthio)pyrimidine-5-carboxamido)phenyl)(methyl)(oxo)-λ⁶-sulfaneylidene)carbamate: To a solution of 4-(4,4-difluoroazepan-1-yl)-6-methyl-2-(methylthio)pyrimidine-5-carboxylic acid (860 mg, 2.70 mmol) in DCM (10 mL) and DMF (20 uL) was added oxalyl chloride (446 mg, 3.51 mmol) at 0° C. The mixture was stirred at room temperature for 5 minutes. Then the solution was added dropwise to a solution of tert-butyl (R)-((3-aminophenyl)(methyl)(oxo)-λ⁶-sulfaneylidene)carbamate (878 mg, 3.24 mmol) and Et₃N (545 mg, 5.40 mmol). The mixture was stirred at room temperature for 1 hour. The resulting mixture was diluted with water (20 mL) and extracted with DCM (40 mL×3). The combined organic layers were washed with water and brine, dried over Na₂SO₄, filtered, concentrated to give the crude. The crude was purified by silica gel column chromatography (PE/EtOAc, 1/1) to afford tert-butyl (R)-((3-(4-(4,4-difluoroazepan-1-yl)-6-methyl-2-(methylthio)pyrimidine-5-carboxamido)phenyl)(methyl)(oxo)-λ⁶-sulfaneylidene)carbamate (750 mg, 48.7%) as a yellow solid. LCMS (ESI) calcd. for C₂₅H₃₄F₂N₅O₄S₂ [M+H]⁺ m/z 570.20, found 570.15.

Step 4: tert-butyl (R)-((3-(4-(4,4-difluoroazepan-1-yl)-6-methyl-2-(methylsulfonyl)pyrimidine-5-carboxamido)phenyl)(methyl)(oxo)-λ⁶-sulfaneylidene)carbamate: A mixture of tert-butyl (R)-((3-(4-(4,4-difluoroazepan-1-yl)-6-methyl-2-(methylthio)pyrimidine-5-carboxamido)phenyl)(methyl)(oxo)-λ⁶-sulfaneylidene)carbamate (750 mg, 1.32 mmol) and m-CPBA (273 mg, 1.58 mmol) in DCM (20 mL) was stirred at room temperature for 2 hours. Then the mixture was concentrated and directly purified by flash column chromatography on silica gel (PE/EtOAc=3/1) to give tert-butyl (R)-((3-(4-(4,4-difluoroazepan-1-yl)-6-methyl-2-(methylsulfonyl)pyrimidine-5-carboxamido)phenyl)(methyl)(oxo)-λ⁶-sulfaneylidene)carbamate (530 mg, 66.7%) as a yellow solid. LCMS (ESI) calcd. for C₂₅H₃₄F₂N₅O₆S₂ [M+H]⁺ m/z 602.19, found 602.15.

Step 5: tert-butyl (R)-((3-(4-(4,4-difluoroazepan-1-yl)-6-methyl-2-morpholinopyrimidine-5-carboxamido)phenyl)(methyl)(oxo)-λ⁶-sulfaneylidene)carbamate: A mixture of tert-butyl (R)-((3-(4-(4,4-difluoroazepan-1-yl)-6-methyl-2-(methylsulfonyl)pyrimidine-5-carboxamido)phenyl)(methyl)(oxo)-λ⁶-sulfaneylidene)carbamate (100 mg, 0.16 mmol) and morpholine (30 mg, 0.33 mmol) in DMF (5 mL) was heated at 100° C. for 6 hours. After the reaction was completed, the mixture was cooled to room temperature. The resulting solution was diluted with water (50 mL) and extracted with EtOAc (30 mL×3). The combined organic phases were washed with brine, dried over Na₂SO₄, concentrated under vacuum. The residue was purified by flash column chromatography on silica gel (PE/EtOAc=4/1) to give tert-butyl (R)-((3-(4-(4,4-difluoroazepan-1-yl)-6-methyl-2-morpholinopyrimidine-5-carboxamido)phenyl)(methyl)(oxo)-λ⁶-sulfaneylidene)carbamate (75 mg, 76.9%) as a yellow solid. LCMS (ESI) calcd. for C₂₈H₃₉F₂N₆O₅S [M+H]⁺ m/z 609.27, found 609.25.

Step 6: (R)-4-(4,4-difluoroazepan-1-yl)-6-methyl-N-(3-(S-methylsulfonimidoyl)phenyl)-2-morpholinopyrimidine-5-carboxamide: A solution of tert-butyl (R)-((3-(4-(4,4-difluoroazepan-1-yl)-6-methyl-2-morpholinopyrimidine-5-carboxamido)phenyl)(methyl)(oxo)-λ⁶-sulfaneylidene)carbamate (75 mg, 0.12 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 40% to 90% MeCN/H₂O containing 0.05% NH₃) to provide (R)-4-(4,4-difluoroazepan-1-yl)-6-methyl-N-(3-(S-methylsulfonimidoyl)phenyl)-2-morpholinopyrimidine-5-carboxamide (26.5 mg, 37.5%) as a white solid. ¹H NMR (400 MHz, DMSO-d₆) δ 10.62 (s, 1H), 8.31 (t, J=1.8 Hz, 1H), 7.86 (d, J=8.8 Hz, 1H), 7.64 (d, J=8.0 Hz, 1H), 7.56 (t, J=7.9 Hz, 1H), 4.20 (s, 1H), 3.70-3.60 (m, 10H), 3.53 (t, J=6.1 Hz, 2H), 3.05 (s, 3H), 2.31-2.14 (m, 5H), 2.04-1.93 (m, 2H), 1.85-1.76 (m, 2H). LCMS (ESI) calcd. for C₂₃H₃₁F₂N₆O₃S [M+H]⁺ m/z 509.22, found 509.20.

### Example 156

### (R)-2-(4,4-difluoroazepan-1-yl)-5-(3,5-dimethylisoxazol-4-yl)-4-methyl-N-(3-(S-methylsulfonimidoyl)phenyl)nicotinamide

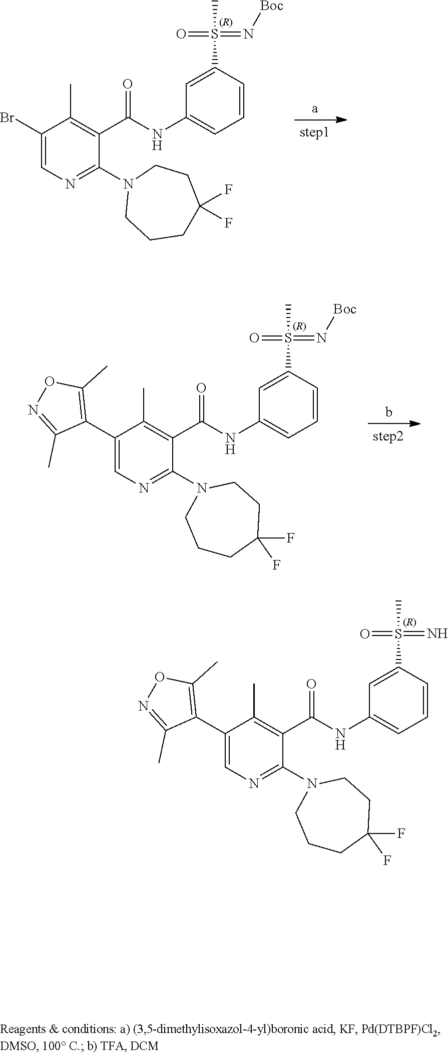

Step 1: tert-butyl (R)-((3-(2-(4,4-difluoroazepan-1-yl)-5-(3,5-dimethylisoxazol-4-yl)-4-methylnicotinamido)phenyl)(methyl)(oxo)-λ⁶--sulfaneylidene)carbamate: A mixture of tert-butyl (R)-((3-(5-bromo-2-(4,4-difluoroazepan-1-yl)-4-methylnicotinamido)phenyl)(methyl)(oxo)-λ⁶-sulfaneylidene)carbamate (100 mg, 0.17 mmol), (3,5-dimethylisoxazol-4-yl)boronic acid (35 mg, 0.25 mmol), KF (29 mg, 0.50 mmol) and Pd(DTBPF)Cl₂ (22 mg, 0.03 mmol) in DMSO (4 mL) and H₂O (1 mL) was heated at 100° C. for 16 hours under an atmosphere of N₂. After the reaction was completed, the mixture was cooled to room temperature. The resulting solution was diluted with water (40 mL) and extracted with EtOAc (40 mL×3). The combined organic phases were washed with brine, dried over Na₂SO₄, concentrated under vacuum. The residue was purified by flash column chromatography on silica gel (PE/EtOAc=1/1) to give tert-butyl (R)-((3-(2-(4,4-difluoroazepan-1-yl)-5-(3,5-dimethylisoxazol-4-yl)-4-methylnicotinamido)phenyl)(methyl)(oxo)-λ⁶-sulfaneylidene)carbamate (64 mg, 62.2%) as a yellow solid. LCMS (ESI) calcd. for C₃₀H₃₈F₂N₅O₅S [M+H]⁺ m/z 618.26, found 618.20.

Step 2: (R)-2-(4,4-difluoroazepan-1-yl)-5-(3,5-dimethylisoxazol-4-yl)-4-methyl-N-(3-(S-methylsulfonimidoyl)phenyl)nicotinamide: A solution of tert-butyl (R)-((3-(2-(4,4-difluoroazepan-1-yl)-5-(3,5-dimethylisoxazol-4-yl)-4-methylnicotinamido)phenyl)(methyl)(oxo)-λ⁶-sulfaneylidene)carbamate (64 mg, 0.16 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (30 mL×3). The combined organic phases were washed with brine, dried over sodium sulfate, concentrated under vacuum. The residue was purified by prep-HPLC (Gemini 5 um C₁₈ column, 150*21.2 mm, eluting with 35% to 90% MeCN/H₂O containing 0.1% NH₃) to provide (R)-2-(4,4-difluoroazepan-1-yl)-5-(3,5-dimethylisoxazol-4-yl)-4-methyl-N-(3-(S-methylsulfonimidoyl)phenyl)nicotinamide (50.3 mg, 91.9% yield) as a white solid. ¹H NMR (400 MHz, DMSO-d₆) δ 10.87 (s, 1H), 8.34 (t, J=1.8 Hz, 1H), 8.02 (s, 1H), 7.96-7.89 (m, 1H), 7.69-7.64 (m, 1H), 7.59 (t, J=7.9 Hz, 1H), 4.23 (s, 1H), 3.70-3.51 (m, 4H), 3.08-3.00 (m, 3H), 2.35-2.20 (m, 5H), 2.14-1.91 (m, 8H), 1.87-1.77 (m, 2H). LCMS (ESI) calcd. for C₂₅H₃₀F₂N₅O₃S [M+H]⁺ m/z 518.21, found 517.95.

### Example 157

### (R)-4-(4,4-difluoroazepan-1-yl)-6-methyl-2-(4-methylpiperazin-1-yl)-N-(3-(S-methylsulfonimidoyl)phenyl)pyrimidine-5-carboxamide

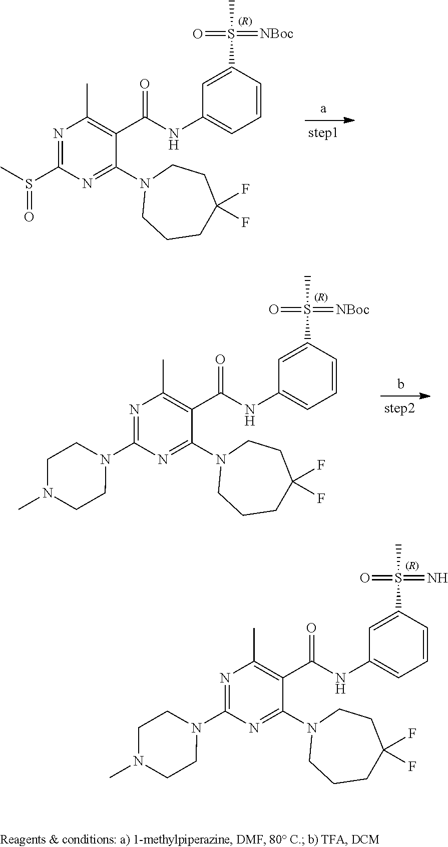

Step 1: tert-butyl (R)-((3-(4-(4,4-difluoroazepan-1-yl)-6-methyl-2-(4-methylpiperazin-1-yl)pyrimidine-5-carboxamido)phenyl)(methyl)(oxo)-λ⁶-sulfaneylidene)carbamate: A mixture of tert-butyl ((1R)-(3-(4-(4,4-difluoroazepan-1-yl)-6-methyl-2-(methylsulfinyl)pyrimidine-5-carboxamido)phenyl)(methyl)(oxo)-λ⁶-sulfaneylidene)carbamate (100 mg, 0.17 mmol) and 1-methylpiperazine (34 mg, 0.34 mmol) in DMF (5 mL) was heated at 80° C. for 6 hours. After the reaction was completed, the mixture was cooled to room temperature. The resulting solution was diluted with water (50 mL) and extracted with EtOAc (30 mL×3). The combined organic phases were washed with brine, dried over Na₂SO₄, concentrated under vacuum. The residue was purified by flash column chromatography on silica gel (PE/EtOAc=4/1) to give tert-butyl (R)-((3-(4-(4,4-difluoroazepan-1-yl)-6-methyl-2-(4-methylpiperazin-1-yl)pyrimidine-5-carboxamido)phenyl)(methyl)(oxo)-λ⁶-sulfaneylidene)carbamate (56 mg, 53%) as a yellow solid. LCMS (ESI) calcd. for C₂₉H₄₂F₂N₇O₄S [M+H]⁺ m/z 622.30, found 622.20.

Step 2: (R)-4-(4,4-difluoroazepan-1-yl)-6-methyl-2-(4-methylpiperazin-1-yl)-N-(3-(S-methylsulfonimidoyl)phenyl)pyrimidine-5-carboxamide: A solution of tert-butyl (R)-((3-(4-(4,4-difluoroazepan-1-yl)-6-methyl-2-(4-methylpiperazin-1-yl)pyrimidine-5-carboxamido)phenyl)(methyl)(oxo)-λ⁶-sulfaneylidene)carbamate (56 mg, 0.09 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 30% to 80% MeCN/H₂O containing 0.05% NH₃) to provide (R)-4-(4,4-difluoroazepan-1-yl)-6-methyl-2-(4-methylpiperazin-1-yl)-N-(3-(S-methylsulfonimidoyl)phenyl)pyrimidine-5-carboxamide (11.50 mg, 24.5%) as a white solid. ¹H NMR (400 MHz, DMSO-d₆) δ 10.61 (s, 1H), 8.31 (t, J=1.8 Hz, 1H), 7.86 (d, J=8.0 Hz, 1H), 7.63 (d, J=8.2 Hz, 1H), 7.55 (t, J=7.9 Hz, 1H), 4.20 (s, 1H), 3.77-3.65 (m, 4H), 3.65-3.50 (m, 4H), 3.05 (s, 3H), 2.37-2.24 (m, 6H), 2.23-2.15 (m, 6H), 2.05-1.91 (m, 2H), 1.86-1.74 (m, 2H). LCMS (ESI) calcd. for C₂₄H₃₄F₂N₇O₂S [M+H]⁺ m/z 522.25, found 522.20.

### Example 158

### (R)-4-(4,4-difluoroazepan-1-yl)-2-(3,3-difluoroazetidin-1-yl)-6-methyl-N-(3-(S-methylsulfonimidoyl)phenyl)pyrimidine-5-carboxamide

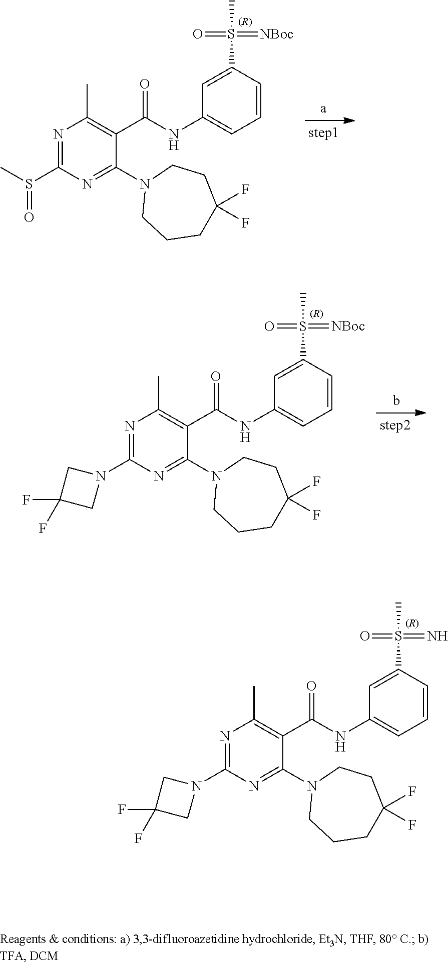

Step 1: tert-butyl (R)-((3-(4-(4,4-difluoroazepan-1-yl)-2-(3,3-difluoroazetidin-1-yl)-6-methylpyrimidine-5-carboxamido)phenyl)(methyl)(oxo)-λ⁶-sulfaneylidene)carbamate: A mixture of tert-butyl ((1R)-(3-(4-(4,4-difluoroazepan-1-yl)-6-methyl-2-(methylsulfinyl)pyrimidine-5-carboxamido)phenyl)(methyl)(oxo)-λ⁶-sulfaneylidene)carbamate (100 mg, 0.17 mmol), 3,3-difluoroazetidine hydrochloride (44 mg, 0.34 mmol) and Et₃N (52 mg, 0.51 mmol) in THE (5 mL) was heated at 80° C. for 16 hours in sealed tube. After the reaction was completed, the mixture was cooled to room temperature. The resulting solution was diluted with water (50 mL) and extracted with EtOAc (30 mL×3). The combined organic layers were washed with brine, dried over Na₂SO₄, concentrated under vacuum. The residue was purified by flash column chromatography on silica gel (PE/EtOAc=4/1) to give tert-butyl (R)-((3-(4-(4,4-difluoroazepan-1-yl)-2-(3,3-difluoroazetidin-1-yl)-6-methylpyrimidine-5-carboxamido)phenyl)(methyl)(oxo)-λ⁶-sulfaneylidene)carbamate (54 mg, 51.6%) as a yellow solid. LCMS (ESI) calcd. for C₂₇H₃₅F₄N₆O₄S [M+H]⁺ m/z 615.24, found 615.15.

Step 2: (R)-4-(4,4-difluoroazepan-1-yl)-2-(3,3-difluoroazetidin-1-yl)-6-methyl-N-(3-(S-methylsulfonimidoyl)phenyl)pyrimidine-5-carboxamide: A solution of tert-butyl (R)-((3-(4-(4,4-difluoroazepan-1-yl)-2-(3,3-difluoroazetidin-1-yl)-6-methylpyrimidine-5-carboxamido)phenyl)(methyl)(oxo)-λ⁶-sulfaneylidene)carbamate (54 mg, 0.08 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 30% to 80% MeCN/H₂O containing 0.05% NH₃) to provide (R)-4-(4,4-difluoroazepan-1-yl)-2-(3,3-difluoroazetidin-1-yl)-6-methyl-N-(3-(S-methylsulfonimidoyl)phenyl)pyrimidine-5-carboxamide (17.20 mg, 38.3% yield) as a white solid. ¹H NMR (400 MHz, DMSO-d₆) δ 10.72 (s, 1H), 8.30 (s, 1H), 7.86 (d, J=7.3 Hz, 1H), 7.70-7.52 (m, 2H), 4.41 (t, J=12.4 Hz, 4H), 4.22 (s, 1H), 3.69-3.53 (m, 4H), 3.06 (s, 3H), 2.34-2.19 (m, 5H), 2.06-1.92 (m, 2H), 1.86-1.74 (m, 2H). LCMS (ESI) calcd. for C₂₂H₂₇F₄N₆O₂S [M+H]⁺ m/z 515.19, found 515.20.

### Example 159

### (R)-2-(4,4-difluoroazepan-1-yl)-5-(1-(difluoromethyl)-1H-pyrazol-4-yl)-4-methyl-N-(3-(S-methylsulfonimidoyl)phenyl)nicotinamide

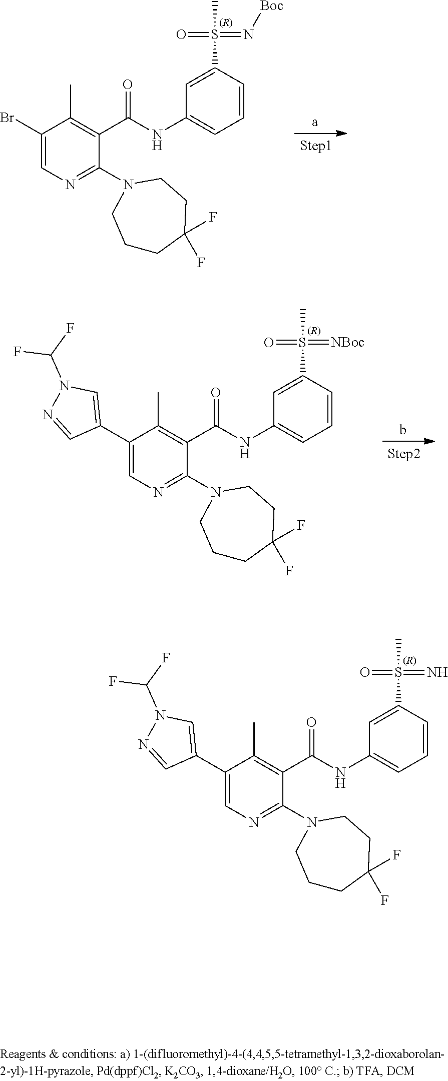

Step 1: tert-butyl (R)-((3-(2-(4,4-difluoroazepan-1-yl)-5-(1,3-dimethyl-1H-pyrazol-4-yl)-4-methylnicotinamido)phenyl)(methyl)(oxo)-λ⁶-sulfaneylidene)carbamate: A mixture of tert-butyl (R)-((3-(5-bromo-2-(4,4-difluoroazepan-1-yl)-4-methylnicotinamido)phenyl)(methyl)(oxo)-λ⁶-sulfaneylidene)carbamate (250 mg, 0.41 mmol), 1-(difluoromethyl)-4-(4,4,5,5-tetramethyl-1,3,2-dioxaborolan-2-yl)-1H-pyrazole (202.5 mg, 0.83 mmol), potassium carbonate (172 mg, 1.2 mmol) and [1,1′-Bis(diphenylphosphino)ferrocene]dichloropalladium (II) (30 mg, 0.04 mmol) in 1,4-dioxane/H₂O (4/1, 15 mL) was heated at 100° C. for 5 h under an atmosphere of N₂. After the reaction was completed, the mixture was cooled to room temperature. The resulting solution was diluted with water (50 mL) and extracted with EtOAc (50 mL×3). The combine organic phases were washed with brine, dried over Na₂SO₄, concentrated under vacuum. The residue was purified by flash column chromatography on silica gel (PE/EtAOc=1/1) to give tert-butyl (R)-((3-(2-(4,4-difluoroazepan-1-yl)-5-(1-(difluoromethyl)-1H-pyrazol-4-yl)-4-methylnicotinamido)phenyl)(methyl)(oxo)-λ⁶-sulfaneylidene)carbamate (260 mg, 98%) as a yellow oil. LCMS (ESI) calcd. for C₂₉H₃₅F₄N₆O₄S [M+H]⁺ m/z 639.24, found 639.15.

Step 2: (R)-2-(4,4-difluoroazepan-1-yl)-5-(1-(difluoromethyl)-1H-pyrazol-4-yl)-4-methyl-N-(3-(S-methylsulfonimidoyl)phenyl)nicotinamide: A solution of tert-butyl (R)-((3-(2-(4,4-difluoroazepan-1-yl)-5-(1-(difluoromethyl)-1H-pyrazol-4-yl)-4-methylnicotinamido)phenyl)(methyl)(oxo)-λ⁶-sulfaneylidene)carbamate (80 mg, 0.13 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 40% to 90% MeCN/H₂O containing 0.05% NH₃) to provide (R)-2-(4,4-difluoroazepan-1-yl)-5-(1-(difluoromethyl)-1H-pyrazol-4-yl)-4-methyl-N-(3-(S-methylsulfonimidoyl)phenyl)nicotinamide (30.6 mg, 43.8%) as a white solid. ¹H NMR (400 MHz, DMSO-d₆) δ 10.88 (s, 1H), 8.48-8.33 (m, 2H), 8.23 (s, 1H), 8.08-7.64 (m, 5H), 7.59 (t, J=7.8 Hz, 1H), 4.23 (s, 1H), 3.69-3.53 (m, 4H), 3.06 (s, 3H), 2.26 (s, 5H), 1.99 (s, 2H), 1.81 (s, 2H). LCMS (ESI) calcd. for C₂₄H₂₇F₄N₆O₂S [M+H]⁺ m/z 539.19, found 539.15

### Example 160

### (R)-2-(4,4-difluoroazepan-1-yl)-5-(1-isopropyl-1H-pyrazol-4-yl)-4-methyl-N-(3-(S-methylsulfonimidoyl)phenyl)nicotinamide

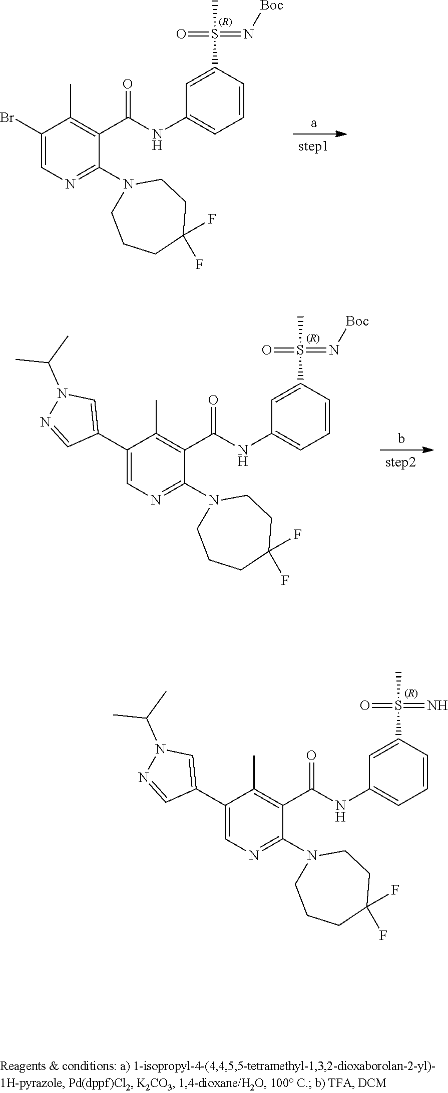

Step 1: tert-butyl (R)-((3-(2-(4,4-difluoroazepan-1-yl)-5-(1-isopropyl-1H-pyrazol-4-yl)-4-methylnicotinamido)phenyl)(methyl)(oxo)-λ⁶-sulfaneylidene)carbamate: To a solution of tert-butyl (R)-((3-(5-bromo-2-(4,4-difluoroazepan-1-yl)-4-methylnicotinamido)phenyl)(methyl)(oxo)-λ⁶-sulfaneylidene)carbamate (260 mg, 0.43 mmol), 1-isopropyl-4-(4,4,5,5-tetramethyl-1,3,2-dioxaborolan-2-yl)-1H-pyrazole (312 mg, 1.32 mmol) and K₂CO₃ (182 mg, 1.32 mmol) in 1,4-dioxane (8 mL) and H₂O (2 mL) was added Pd(dppf)Cl₂ (28 mg, 0.03 mmol). Then the mixture was heated at 100° C. for 12 hours under N₂. LCMS showed the reaction was completed. The mixture was diluted with water and extracted with EtOAc (3×50 mL). The combined organic phases were washed with brine, dried with Na₂SO₄, and concentrated under vacuum. The residue was purified by silica gel column chromatography (eluting with MeOH/DCM, 0 to 10%) to give tert-butyl (R)-((3-(2-(4,4-difluoroazepan-1-yl)-5-(1-isopropyl-1H-pyrazol-4-yl)-4-methylnicotinamido)phenyl)(methyl)(oxo)-λ⁶-sulfaneylidene)carbamate (250 mg, 39.68%) as a yellow oil. LCMS (ESI) calcd. for C₃₁H₄₁F₂N₆O₄S [M+H]⁺ m/z 631.28, found 631.20.

Step 2: (R)-2-(4,4-difluoroazepan-1-yl)-5-(1-isopropyl-1H-pyrazol-4-yl)-4-methyl-N-(3-(S-methylsulfonimidoyl)phenyl)nicotinamide: A solution of tert-butyl (R)-((3-(2-(4,4-difluoroazepan-1-yl)-5-(1-isopropyl-1H-pyrazol-4-yl)-4-methylnicotinamido)phenyl)(methyl)(oxo)-λ⁶--sulfaneylidene)carbamate (120 mg, 0.19 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 40% to 90% MeCN/H₂O containing 0.05% NH₃) to provide (59.9 mg, 59.5%) as a white solid. ¹H NMR (400 MHz, DMSO-d₆) δ 10.84 (s, 1H), 8.37 (t, J=1.8 Hz, 1H), 8.19 (s, 1H), 7.96 (s, 1H), 7.90 (d, J=8.8 Hz, 1H), 7.67 (d, J=8.0 Hz, 1H), 7.59 (dd, J=13.4, 5.4 Hz, 2H), 4.55-4.49 (m, 1H), 4.22 (s, 1H), 3.63-3.52 (m, 4H), 3.06 (s, 3H), 2.37-2.17 (m, 5H), 2.15-1.92 (m, 2H), 1.87-1.75 (m, 2H), 1.46 (s, 3H), 1.45 (s, 3H). LCMS (ESI) calcd. for C₂₆H₃₃F₂N₆O₂S [M+H]⁺ m/z 531.23, found 531.20.

### Example 161

### (R)-4-(4,4-difluoroazepan-1-yl)-6-methyl-N-(3-(S-methylsulfonimidoyl)phenyl)-2-(piperazin-1-yl)pyrimidine-5-carboxamide

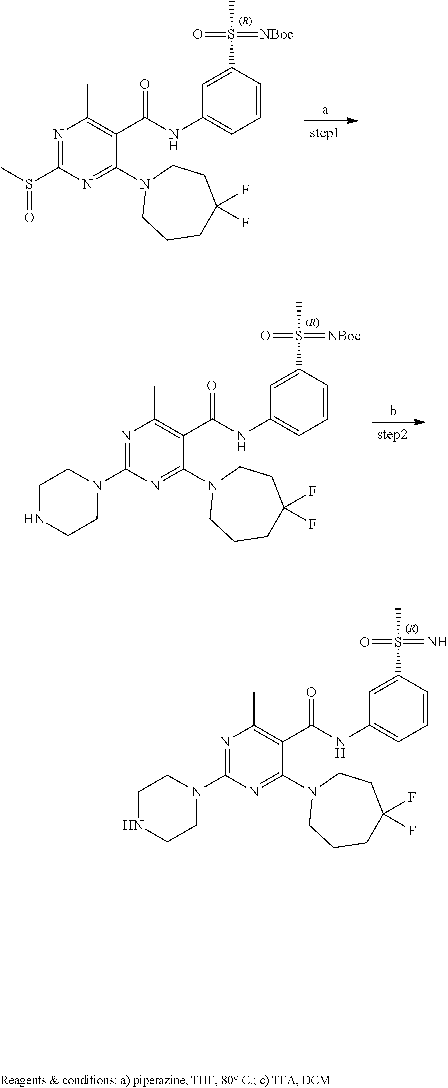

Step 1: tert-butyl (R)-((3-(4-(4,4-difluoroazepan-1-yl)-6-methyl-2-(piperazin-1-yl)pyrimidine-5-carboxamido)phenyl)(methyl)(oxo)-λ⁶-sulfaneylidene)carbamate: A mixture of tert-butyl ((1R)-(3-(4-(4,4-difluoroazepan-1-yl)-6-methyl-2-(methylsulfinyl)pyrimidine-5-carboxamido)phenyl)(methyl)(oxo)-λ⁶-sulfaneylidene)carbamate (100 mg, 0.17 mmol) and piperazine (29 mg, 0.34 mmol) in THE (5 mL) was heated at 80° C. for 16 hours in a sealed tube. After the reaction was completed, the mixture was cooled to room temperature. The resulting solution was diluted with water (50 mL) and extracted with EtOAc (30 mL×3). The combined organic phases were washed with brine, dried over Na₂SO₄, concentrated under vacuum. The residue was purified by flash column chromatography on silica gel (PE/EtOAc=4/1) to give tert-butyl ((1R)-(3-(4-(4,4-difluoroazepan-1-yl)-6-methyl-2-(methylsulfinyl)pyrimidine-5-carboxamido)phenyl)(methyl)(oxo)-λ⁶-sulfaneylidene)carbamate (53 mg, 51.6%) as a yellow solid. LCMS (ESI) calcd. for C₂₈H₄₀F₂N₇O₄S [M+H]⁺ m/z 608.29, found 608.20.

Step 2: (R)-4-(4,4-difluoroazepan-1-yl)-6-methyl-N-(3-(S-methylsulfonimidoyl)phenyl)-2-(piperazin-1-yl)pyrimidine-5-carboxamide. A solution of tert-butyl ((1R)-(3-(4-(4,4-difluoroazepan-1-yl)-6-methyl-2-(methylsulfinyl)pyrimidine-5-carboxamido)phenyl)(methyl)(oxo)-λ⁶-sulfaneylidene)carbamate (54 mg, 0.08 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 30% to 80% MeCN/H₂O containing 0.05% NH₃) to provide (R)-4-(4,4-difluoroazepan-1-yl)-6-methyl-N-(3-(S-methylsulfonimidoyl)phenyl)-2-(piperazin-1-yl)pyrimidine-5-carboxamide (13.30 mg, 32.7%) as a white solid. ¹H NMR (400 MHz, DMSO-d₆) δ 10.62 (s, 1H), 8.31 (s, 1H), 7.86 (d, J=8.1 Hz, 1H), 7.69-7.49 (m, 2H), 4.20 (s, 1H), 3.68-3.48 (m, 8H), 3.05 (s, 3H), 2.79-2.58 (m, 4H), 2.32-2.16 (m, 5H), 2.05-1.90 (m, 2H), 1.84-1.73 (m, 2H). LCMS (ESI) calcd. for C₂₃H₃₂F₂N₇O₂S [M+H]⁺ m/z 508.23, found 508.10.

### Example 162

### (R)-5-chloro-2-(4,4-difluoroazepan-1-yl)-4-methyl-6-(1-methyl-1H-pyrazol-4-yl)-N-(3-(S-methylsulfonimidoyl)phenyl)nicotinamide

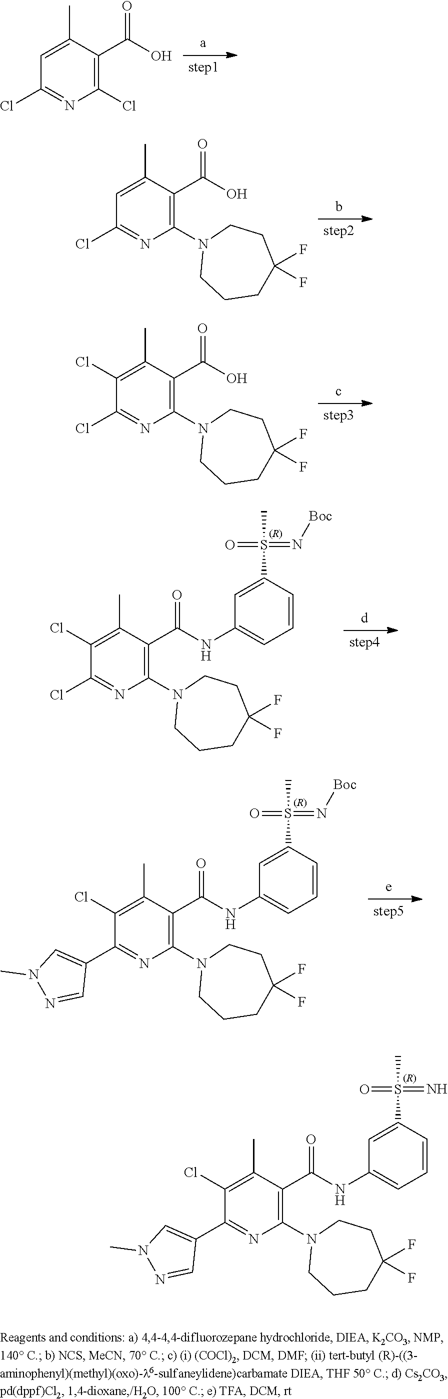

Step 1: 6-chloro-2-(4,4-difluoroazepan-1-yl)-4-methylnicotinic acid: A solution of 2,6-dichloro-4-methylnicotinic acid (3.00 g, 14.56 mmol), 4,4-4,4-difluoroazepane hydrochloride (3.00 g, 17.47 mmol), K₂CO₃ (10.06 g, 72.81 mmol) and DIEA (9.41 g, 63.6 mmol) in NMP (40 mL) was heated at 140° C. for 6 hours. LCMS showed the reaction was completed. The mixture was diluted with water. The mixture was adjusted to pH=3-4 with 2N HCl. The solution was extracted with EtOAc. The combined organic layers were dried over anhydrous Na₂SO₄ and concentrated to afford the crude. The residue was purified by flash column chromatography on silica gel (PE/EtOAc=2/1) to provide 6-chloro-2-(4,4-difluoroazepan-1-yl)-4-methylnicotinic acid (3.20 g, 72.12%) as a white solid. LCMS (ESI) calcd. for C₁₃H₁₆ClF₂N₂O₂ [M+H]⁺ m/z 305.09, found 305.05.

Step 2: 5,6-dichloro-2-(4,4-difluoroazepan-1-yl)-4-methylnicotinic acid: A solution of 6-chloro-2-(4,4-difluoroazepan-1-yl)-4-methylnicotinic acid (2.00 g, 6.56 mmol) in MeCN (40 mL) was added NCS (964 mg, 7.22 mmol) at room temperature. The reaction mixture was heated to 70° C. for 5 hours. LCMS showed the reaction was completed. The mixture was diluted with water and extracted with EtOAc (80 mL×3). The combined organic layers were dried over anhydrous Na₂SO₄ and concentrated to afford the crude. The residue was purified by flash column chromatography on silica gel (PE/EtOAc=4/1) to provide 5,6-dichloro-2-(4,4-difluoroazepan-1-yl)-4-methylnicotinic acid (1.10 g, 49.4%) as a brown solid. LCMS (ESI) calcd. for C₁₃H₁₅C₁₂F₂N₂O₂ [M+H]⁺ m/z 339.05, found 339.05.

Step 3: tert-butyl (R)-((3-(5,6-dichloro-2-(4,4-difluoroazepan-1-yl)-4-methylnicotinamido)phenyl)(methyl)(oxo)-λ⁶-sulfaneylidene)carbamate: A solution of 5,6-dichloro-2-(4,4-difluoroazepan-1-yl)-4-methylnicotinic acid (200 mg, 0.59 mmol) in DCM (5 mL) was added DMF (4 drop) and oxalyl chloride (112 mg, 0.88 mmol) at room temperature. The reaction mixture was stirred at room temperature for 5 minutes. Then the solution was added dropwise to a solution of tert-butyl (R)-((3-aminophenyl)(methyl)(oxo)-λ⁶-sulfaneylidene)carbamate (653 mg, 2.42 mmol) and DIEA (229 mg, 1.77 mmol) in THE (10 mL). The mixture was heated to 50° C. for 5 hours. After the reaction was completed, the resulting solution was diluted with water (80 mL) and extracted with DCM (10 mL×3). The combined organic phases were washed with brine, dried over sodium sulfate, concentrated under vacuum. The residue was purified by flash column chromatography on silica gel (DCM/MeOH=10/1) to give tert-butyl (R)-((3-(5,6-dichloro-2-(4,4-difluoroazepan-1-yl)-4-methylnicotinamido)phenyl)(methyl)(oxo)-λ⁶-sulfaneylidene)carbamate (95 mg, 27.2%). LCMS (ESI) calcd. for C₂₅H₃₁C₁₂F₂N₄O₄S [M+H]⁺ m/z 591.14, found 591.10.

Step 4: tert-butyl (R)-((3-(5-chloro-2-(4,4-difluoroazepan-1-yl)-4-methyl-6-(1-methyl-1H-pyrazol-4-yl)nicotinamido)phenyl)(methyl)(oxo)-λ⁶-sulfaneylidene)carbamate: A solution of tert-butyl (R)-((3-(5,6-dichloro-2-(4,4-difluoroazepan-1-yl)-4-methylnicotinamido)phenyl)(methyl)(oxo)-λ⁶-sulfaneylidene)carbamate (60 mg, 0.10 mmol), (1-methyl-1H-pyrazol-4-yl)boronic acid (25 mg, 0.20 mmol), Cs₂CO₃ (98 mg, 0.30 mmol) and Pd(dppf)Cl₂ (13 mg, 0.02 mmol) in dioxane (4 mL) and H₂O (1 mL) was heated at 100° C. for 6 hours. LCMS showed the reaction was completed. The mixture was diluted with water (30 mL) and extracted with EtOAc (20 mL×3). The combined organic layers were washed with brine, dried over sodium sulfate, concentrated under vacuum. The residue was purified by flash column chromatography on silica gel (PE/EtOAc=10/1 to 1/1) to provide tert-butyl (R)-((3-(5-chloro-2-(4,4-difluoroazepan-1-yl)-4-methyl-6-(1-methyl-1H-pyrazol-4-yl)nicotinamido)phenyl)(methyl)(oxo)-λ⁶-sulfaneylidene)carbamate (49 mg, 90.03%) as yellow oil. LCMS (ESI) calcd. for C₂₉H₃₆ClF₂N₆O₄S [M+H]⁺ m/z 637.22, found 637.15.

Step 5: (R)-5-chloro-2-(4,4-difluoroazepan-1-yl)-4-methyl-6-(1-methyl-1H-pyrazol-4-yl)-N-(3-(S-methylsulfonimidoyl)phenyl)nicotinamide: A solution of tert-butyl (R)-((3-(5-chloro-2-(4,4-difluoroazepan-1-yl)-4-methyl-6-(1-methyl-1H-pyrazol-4-yl)nicotinamido)phenyl)(methyl)(oxo)-λ⁶-sulfaneylidene)carbamate (49 mg, 0.077 mmol) in DCM (2 mL) was added TFA (0.2 mL) at room temperature. The reaction mixture was stirred at room temperature for 1 hour. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 20% to 95% MeCN/H₂O containing 0.05% NH₃·H₂O) to provide (R)-5-chloro-2-(4,4-difluoroazepan-1-yl)-4-methyl-6-(1-methyl-1H-pyrazol-4-yl)-N-(3-(S-methylsulfonimidoyl)phenyl)nicotinamide (20.4 mg, 49.5%) as a white solid. ¹H NMR (400 MHz, DMSO-d₆) δ 10.87 (s, 1H), 8.45 (s, 1H), 8.33 (t, J=1.8 Hz, 1H), 8.11 (d, J=0.6 Hz, 1H), 7.89 (d, J=9.0 Hz, 1H), 7.69-7.54 (m, 2H), 4.23 (s, 1H), 3.93 (s, 3H), 3.70-3.51 (m, 4H), 3.06 (s, 3H), 2.36-2.26 (m, 5H), 2.06-1.94 (m, 2H), 1.87-1.78 (m, 2H). LCMS (ESI) calcd. for C₂₄H₂₈ClF₂N₆O₂S [M+H]⁺ m/z 537.17, found 537.10.

### Example 163 and 164

### (S)-2-(4,4-difluoroazepan-1-yl)-4-methyl-N-(3-(S-methylsulfonimidoyl)phenyl)-5-(trifluoromethyl)nicotinamide and (R)-2-(4,4-difluoroazepan-1-yl)-4-methyl-N-(3-(S-methylsulfonimidoyl)phenyl)-5-(trifluoromethyl)nicotinamide

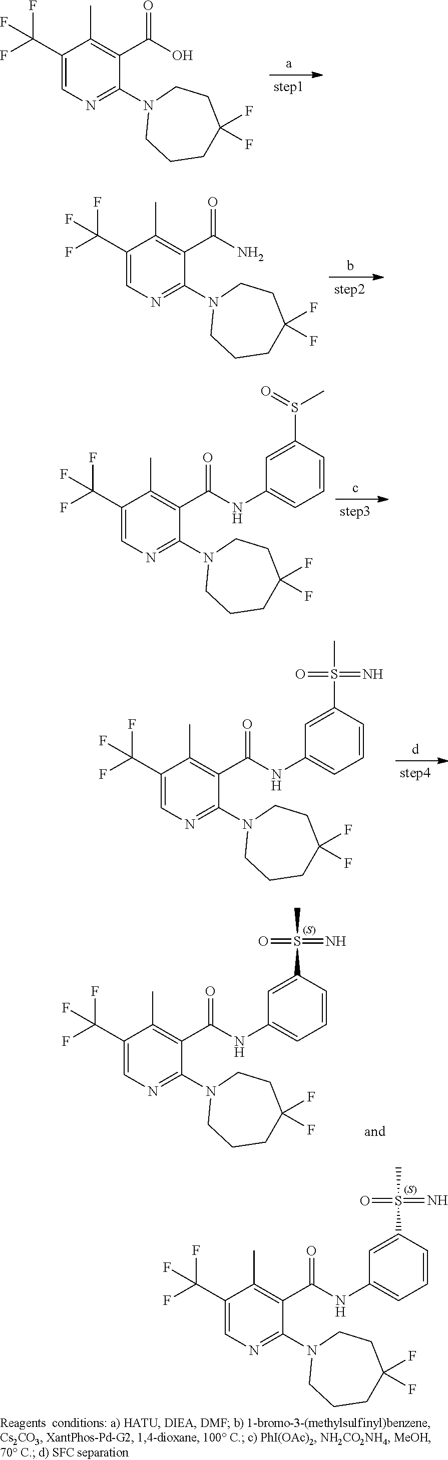

Step 1: 2-(4,4-difluoroazepan-1-yl)-4-methyl-5-(trifluoromethyl)nicotinamide: A solution of 2-(4,4-difluoroazepan-1-yl)-4-methyl-5-(trifluoromethyl)nicotinic acid (500 mg, 1.48 mmol) in DMF (20 mL) was added HATU (1.03 g, 1.92 mmol), DIEA (856 mg, 4.74 mmol) and NH₃-MeOH (10 mL) at room temperature. The mixture was stirred at room temperature for 16 hours. After the reaction was completed, the mixture was diluted with water (100 mL) and extracted with DCM (40 mL×3). The combined organic layers were washed with brine, dried over sodium sulfate, concentrated under vacuum. The residue was purified by flash column chromatography on silica gel (PE/EtOAc=1/1) to give 2-(4,4-difluoroazepan-1-yl)-4-methyl-5-(trifluoromethyl)nicotinamide (410 mg, 82.0%) as a white solid. LCMS (ESI) calcd. for C₁₄H₁₇F₅N₃O [M+H]⁺ m/z 338.13, found 338.00.

Step 2: 2-(4,4-difluoroazepan-1-yl)-4-methyl-N-(3-(methylsulfinyl)phenyl)-5-(trifluoromethyl)nicotinamide: A solution of 2-(4,4-difluoroazepan-1-yl)-4-methyl-5-(trifluoromethyl)nicotinamide (400 mg, 1.18 mmol) in dioxane (10 mL) was added 1-bromo-3-(methylsulfinyl)benzene (1.20 g, 3.08 mmol), Cs₂CO₃ (2.03 g, 3.55 mmol) and Xantphos-Pd-G2 (180 mg, 0.12 mmol) at room temperature. The reaction mixture was stirred at 100° C. for 16 hours under N₂. After the reaction was completed, the mixture was diluted with water (50 mL) and extracted with DCM (40 mL×3). The combine organic phases were washed with brine, dried over sodium sulfate, concentrated under vacuum. The residue was purified by flash column chromatography on silica gel (PE/EtOAc=1/1) to give 2-(4,4-difluoroazepan-1-yl)-4-methyl-N-(3-(methylsulfinyl)phenyl)-5-(trifluoromethyl)nicotinamide (280 mg, 49.73%) as a white solid. LCMS (ESI) calcd. for C₂₁H₂₃F₅N₃O₂S [M+H]⁺ m/z 476.15, found 475.90.

Step 3: 2-(4,4-difluoroazepan-1-yl)-4-methyl-N-(3-(S-methylsulfonimidoyl)phenyl)-5-(trifluoromethyl)nicotinamide: To a solution of 2-(4,4-difluoroazepan-1-yl)-4-methyl-N-(3-(methylsulfinyl)phenyl)-5-(trifluoromethyl)nicotinamide (280 mg, 0.59 mmol) in MeOH (10 mL) was added PhI(OAc)₂ (670 mg, 1.47 mmol) and ammonium carbamate (195 mg, 1.76 mmol) at room temperature. The reaction mixture was heated at 70° C. for 2 hours. After the reaction was completed, the mixture was cooled to room temperature, diluted with water (50 mL) and extracted with DCM (30 mL×3). The combined organic phases were washed with brine, dried over sodium sulfate, concentrated under vacuum. The residue was purified by flash column chromatography on silica gel (PE/EtOAc=1/1) to give the crude. The residue was further purified by prep-HPLC (Gemini 5 um C₁₈ column, 150*21.2 mm, eluting with 30% to 90% MeCN/H₂O containing 0.1% formic acid) to afford 2-(4,4-difluoroazepan-1-yl)-4-methyl-N-(3-(S-methylsulfonimidoyl)phenyl)-5-(trifluoromethyl)nicotinamide (120 mg, 41.7%) as a white solid. LCMS (ESI) calcd. for C₂₁H₂₄F₅N₄O₂S [M+H]⁺ m/z 491.16, found 491.10.

Step 4: (R)-2-(4,4-difluoroazepan-1-yl)-4-methyl-N-(3-(S-methylsulfonimidoyl)phenyl)-5-(trifluoromethyl)nicotinamide and (S)-2-(4,4-difluoroazepan-1-yl)-4-methyl-N-(3-(S-methylsulfonimidoyl)phenyl)-5-(trifluoromethyl)nicotinamide: 2-(4,4-difluoroazepan-1-yl)-4-methyl-N-(3-(S-methylsulfonimidoyl)phenyl)-5-(trifluoromethyl)nicotinamide (120 mg, 0.24 mmol) purified by prep-Chiral-SFC (chiralpak-AS-H, 150*21.2 mm, eluting with 30% CO₀₂-MeOH containing 0.1% NH₃). The first elution was lyophilized to afford (S)-2-(4,4-difluoroazepan-1-yl)-4-methyl-N-(3-(S-methylsulfonimidoyl)phenyl)-5-(trifluoromethyl)nicotinamide (63.5 mg, 52.9%) as a white solid. ¹H NMR (400 MHz, DMSO-d₆) δ 10.95 (s, 1H), 8.45 (s, 1H), 8.31 (s, 1H), 7.90 (d, J=8.4 Hz, 1H), 7.69 (d, J=8 Hz, 1H), 7.62-7.58 (m, 1H), 4.23 (s, 1H), 3.74-3.71 (m, 2H), 3.64-3.61 (m, 2H), 3.06 (s, 3H), 2.32 (s, 3H), 2.29-2.25 (m, 2H), 2.03-1.96 (m, 2H), 1.86-1.81 (m, 2H). LCMS (ESI) calcd. for C₂₁H₂₄F₅N₄O₂S [M+H]⁺ m/z 491.16, found 491.05. The second elution was lyophilized to afford (R)-2-(4,4-difluoroazepan-1-yl)-4-methyl-N-(3-(S-methylsulfonimidoyl)phenyl)-5-(trifluoromethyl)nicotinamide(56.2 mg, 46.8%) as a white solid. ¹H NMR (400 MHz, DMSO-d₆) δ 10.95 (s, 1H), 8.45 (s, 1H), 8.31 (s, 1H), 7.90 (d, J=8 Hz, 1H), 7.69 (d, J=8 Hz, 1H), 7.62-7.58 (m, 1H), 4.23 (s, 1H), 3.74-3.71 (m, 2H), 3.64-3.61 (m, 2H), 3.06 (s, 3H), 2.32 (s, 3H), 2.29-2.25 (m, 2H), 2.07-1.96 (m, 2H), 1.86-1.81 (m, 2H). LCMS (ESI) calcd. for C₂₁H₂₄F₅N₄O₂S [M+H]⁺ m/z 491.16, found 491.05.

### Example 165

### 2-(4,4-difluoroazepan-1-yl)-4-methyl-5-(1-methyl-1H-pyrazol-4-yl)-N-(3-sulfamidimidoylphenyl)nicotinamide

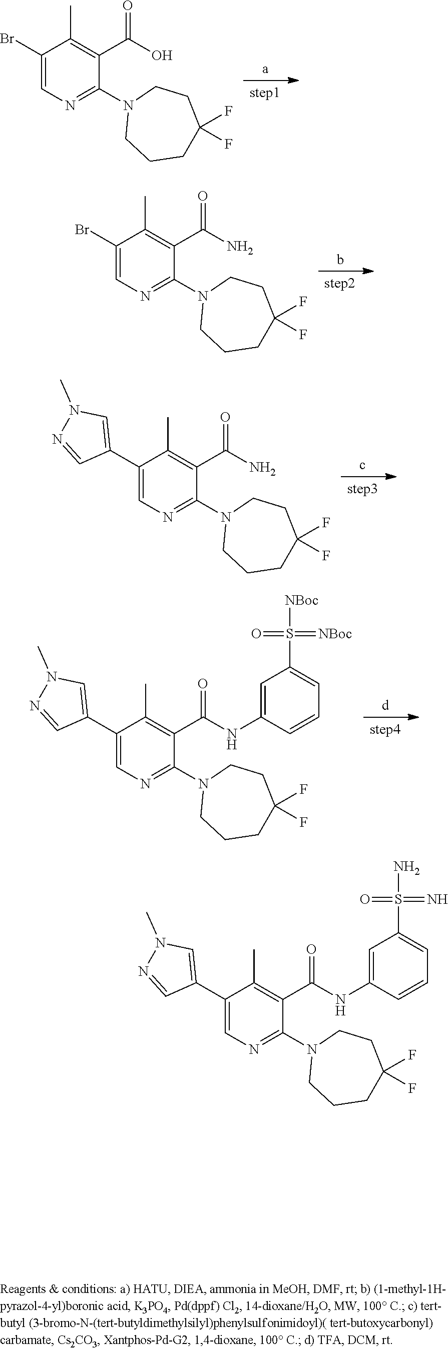

Step 1: 5-bromo-2-(4,4-difluoroazepan-1-yl)-4-methylnicotinamide: To a solution of 5-bromo-2-(4,4-difluoroazepan-1-yl)-4-methylnicotinic acid (1 g, 2.8 mmol) in DMF (10 mL) was added HATU (1.4 g, 3.7 mmol), DIEA (1.2 g, 9 mmol) and NH₃-MeOH (10 mL) at room temperature. The mixture was stirred at room temperature for 16 hours. After the reaction was completed, the mixture was diluted with water (80 mL) and extracted with DCM (30 mL×3). The combined organic layers were washed with brine, dried over sodium sulfate, concentrated under vacuum. The residue was purified by flash column chromatography on silica gel (PE/EtOAc=1/1) to give 5-bromo-2-(4,4-difluoroazepan-1-yl)-4-methylnicotinamide (800 mg, 82.13%) as a white solid. LCMS (ESI) calcd. for C₁₃H₁₇BrF₂N₃O [M+H]⁺ m/z 350.05, found 349.90.

Step 2: 2-(4,4-difluoroazepan-1-yl)-4-methyl-5-(1-methyl-1H-pyrazol-4-yl)nicotinamide: A mixture of 5-bromo-2-(4,4-difluoroazepan-1-yl)-4-methylnicotinamide (800 mg, 2.30 mmol) and (1-methyl-1H-pyrazol-4-yl)boronic acid (725 mg, 5.75 mmol), K₃PO₄ (1.59 g, 6.9 mmol) and [1,1′-Bis(diphenylphosphino)ferrocene]dichloropalladium (II) (168 mg, 0.23 mmol) in 1,4-dioxane/H₂O (10/1, 20 mL) was heated at 100° C. in a microwave reactor for 6 hours under an atmosphere of N₂. The reaction was monitored by LCMS. After the reaction was completed, the mixture was cooled to room temperature. The resulting solution was diluted with water (80 mL) and extracted with DCM (20 mL×3). The combined organic layers were washed with brine, dried over Na₂SO₄, concentrated under vacuum. The residue was purified by flash column chromatography on silica gel (PE/EtAOc=3/1) to give 2-(4,4-difluoroazepan-1-yl)-4-methyl-5-(1-methyl-1H-pyrazol-4-yl)nicotinamide (650 mg, 76.5%) as a white oil. LCMS (ESI) calcd. for C₁₇H₂₂F₂N₅O [M+H]⁺ m/z 350.18, found 350.00.

Step 3: tert-butyl (tert-butoxycarbonyl)(3-(2-(4,4-difluoroazepan-1-yl)-4-methyl-5-(1-methyl-1H-pyrazol-4-yl)nicotinamido)phenylsulfonimidoyl)carbamate: To a solution of 2-(4,4-difluoroazepan-1-yl)-4-methyl-5-(1-methyl-1H-pyrazol-4-yl)nicotinamide (200 mg, 0.57 mmol) in dioxane (10 mL) was added tert-butyl (3-bromo-N-(tert-butyldimethylsilyl)phenylsulfonimidoyl)(tert-butoxycarbonyl)carbamate (313 mg, 0.57 mmol), cesium carbonate (484 mg, 1.49 mmol) and Xantphos-Pd-G2 (101 mg, 0.12 mmol) at room temperature. The reaction mixture was stirred at 100° C. for 16 hours under nitrogen. The reaction was monitored by LCMS. After the reaction was completed, the filtrate was diluted with water (80 mL) and extracted with EtOAc (50 mL×3). The combine organic phases were washed with brine, dried over sodium sulfate, concentrated under vacuum. The residue was purified by flash column chromatography on silica gel (PE/EtOAc=1/1) to give tert-butyl (tert-butoxycarbonyl)(3-(2-(4,4-difluoroazepan-1-yl)-4-methyl-5-(1-methyl-1H-pyrazol-4-yl)nicotinamido)phenylsulfonimidoyl)carbamate (150 mg, 37.31%) as a white solid. LCMS (ESI) calcd. for C₃₃H₄₄F₂N₇O₆S [M+H]⁺ m/z 704.31, found 704.20.

Step 4: 2-(4,4-difluoroazepan-1-yl)-4-methyl-5-(1-methyl-1H-pyrazol-4-yl)-N-(3-sulfamidimidoylphenyl)nicotinamide. A solution of tert-butyl (tert-butoxycarbonyl)(3-(2-(4,4-difluoroazepan-1-yl)-4-methyl-5-(1-methyl-1H-pyrazol-4-yl)nicotinamido)phenylsulfonimidoyl)carbamate (150 mg, 0.21 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (30 mL×3). The combined organic phases were washed with brine, dried over sodium sulfate, concentrated under vacuum. The residue was purified by prep-HPLC (Gemini 5 um C₁₈ column, 150*21.2 mm, eluting with 35% to 90% MeCN/H₂O containing 0.1% NH₃) to provide 2-(4,4-difluoroazepan-1-yl)-4-methyl-5-(1-methyl-1H-pyrazol-4-yl)-N-(3-sulfamidimidoylphenyl)nicotinamide (35.4 mg, 33.1%) as a white solid. ¹H NMR (400 MHz, DMSO-d₆) δ 10.77 (s, 1H), 8.37 (s, 1H), 8.16 (s, 1H), 7.89 (s, 1H), 7.75 (d, J=8 Hz, 1H), 7.65 (d, J=8 Hz, 1H), 7.60 (s, 1H), 7.50-7.46 (m, 1H), 5.80 (bs, 2H), 3.88 (s, 3H), 3.63-3.60 (m, 2H), 3.57-3.54 (m, 2H), 2.32-2.34 (m, 2H), 2.24 (s, 3H), 2.02-1.99 (m, 2H), 1.81-1.79 (m, 2H). LCMS (ESI) calcd. for C₂₃H₂₈F₂N₇O₂S [M+H]⁺ m/z 504.20, found 504.10.

### Example 166

### (R)-5-(4-chloro-3-fluorophenyl)-2-(4,4-difluoroazepan-1-yl)-4-methyl-N-(3-(S-methylsulfonimidoyl)phenyl)nicotinamide

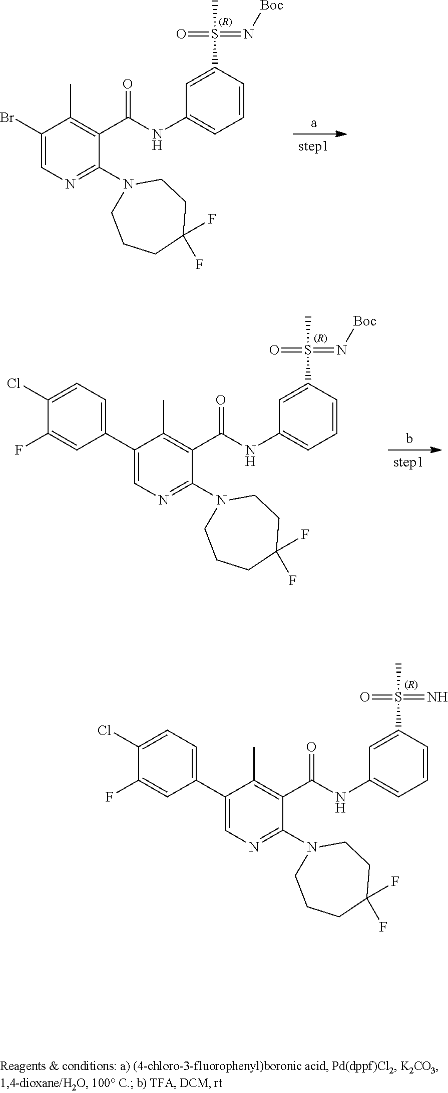

Step 1: tert-butyl (R)-((3-(5-(4-chloro-3-fluorophenyl)-2-(4,4-difluoroazepan-1-yl)-4-methylnicotinamido)phenyl)(methyl)(oxo)-λ⁶-sulfaneylidene)carbamate: A mixture of tert-butyl (R)-((3-(5-bromo-2-(4,4-difluoroazepan-1-yl)-4-methylnicotinamido)phenyl)(methyl)(oxo)-λ⁶-sulfaneylidene)carbamate (150 mg, 0.25 mmol), (4-chloro-3-fluorophenyl)boronic acid (87 mg, 0.5 mmol), K₂CO₃ (69 mg, 0.5 mmol) and Pd(dppf)Cl₂ (15 mg, 0.02 mmol) in 1,4-dioxane/H₂O (4/1, 10 mL) was heated at 100° C. for 16 hours under an atmosphere of N₂. The reaction was monitored by LCMS. After the reaction was completed, the mixture was cooled to room temperature. The resulting solution was diluted with water (80 mL) and extracted with EA (40 mL×3). The combined organic layers were washed with brine, dried over Na₂SO₄, and concentrated under vacuum. The residue was purified by flash column chromatography on silica gel (PE/EtOAc=2/1 to 1/1) to give tert-butyl (R)-((3-(5-(4-chloro-3-fluorophenyl)-2-(4,4-difluoroazepan-1-yl)-4-methylnicotinamido)phenyl)(methyl)(oxo)-λ⁶-sulfaneylidene)carbamate (90 mg, 55.3%) as a yellow solid. LCMS (ESI) calcd. for C₃₁H₃₅ClF₃N₄O₄S [M+H]⁺ m/z 651.20, found 651.10.

Step 2: (R)-5-(4-chloro-3-fluorophenyl)-2-(4,4-difluoroazepan-1-yl)-4-methyl-N-(3-(S-methylsulfonimidoyl)phenyl)nicotinamide: A solution of tert-butyl (R)-((3-(5-(4-chloro-3-fluorophenyl)-2-(4,4-difluoroazepan-1-yl)-4-methylnicotinamido)phenyl)(methyl)(oxo)-λ⁶-sulfaneylidene)carbamate (90 mg, 0.14 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, and concentrated under vacuum. The residue was purified by prep-HPLC (Gemini 5 um C₁₈ column, 150*21.2 mm, eluting with 50% to 95% MeCN/H₂O containing 0.05% NH₃) to (R)-5-(4-chloro-3-fluorophenyl)-2-(4,4-difluoroazepan-1-yl)-4-methyl-N-(3-(S-methylsulfonimidoyl)phenyl)nicotinamide (51.3 mg, 71.5%) as a white solid. ¹H NMR (400 MHz, DMSO-d₆) δ 10.88 (s, 1H), 8.36 (d, J=1.8 Hz, 1H), 8.12 (s, 1H), 7.90 (d, J=8.1 Hz, 1H), 7.71-7.63 (m, 2H), 7.58 (t, J=7.9 Hz, 1H), 7.45 (dd, J=10.3, 1.9 Hz, 1H), 7.22 (dd, J=8.3, 1.6 Hz, 1H), 4.23 (s, 1H), 3.71-3.55 (m, 4H), 3.06 (s, 3H), 2.33-2.22 (m, 2H), 2.17 (s, 3H), 2.06-1.94 (m, 2H), 1.86-1.78 (m, 2H). LCMS (ESI) calcd. for C₂₆H₂₇ClF₃N₄O₂S [M+H]⁺ m/z 551.15, found 551.05.

### Example 167

### (R)-3-(4,4-difluoroazepan-1-yl)-6-(4-(difluoromethyl)phenyl)-5-methyl-N-(3-(S-methylsulfonimidoyl)phenyl)pyridazine-4-carboxamide

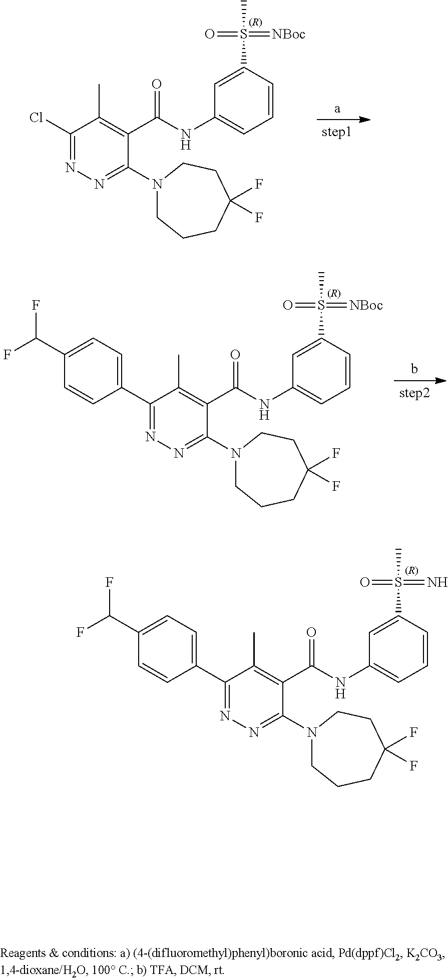

Step 1: tert-butyl (R)-((3-(3-(4,4-difluoroazepan-1-yl)-6-(4-(difluoromethyl)phenyl)-5-methylpyridazine-4-carboxamido)phenyl)(methyl)(oxo)-λ⁶-sulfaneylidene)carbamate: A mixture of tert-butyl (R)-((3-(6-chloro-3-(4,4-difluoroazepan-1-yl)-5-methylpyridazine-4-carboxamido)phenyl)(methyl)(oxo)-λ⁶-sulfaneylidene)carbamate (150 mg, 0.27 mmol), (4-(difluoromethyl)phenyl)boronic acid (69 mg, 0.41 mmol), K₂CO₃ (112 mg, 0.81 mmol) and Pd(dppf)Cl₂ (18 mg, 0.02 mmol) in dioxane (8 mL) and H₂O (2 mL) was heated at 100° C. for 16 hours under an atmosphere of N₂. After the reaction was completed, the mixture was cooled to room temperature. The resulting solution was diluted with water (40 mL) and extracted with EtOAc (30 mL×3). The combined organic phases were washed with brine, dried over Na₂SO₄, concentrated under vacuum. The residue was purified by flash column chromatography on silica gel (PE/EtOAc=1/1) to give tert-butyl (R)-((3-(3-(4,4-difluoroazepan-1-yl)-6-(4-(difluoromethyl)phenyl)-5-methylpyridazine-4-carboxamido)phenyl)(methyl)(oxo)-λ⁶-sulfaneylidene)carbamate (90 mg, 51.13%) as a yellow solid. LCMS (ESI) calcd. for C₃₁H₃₆F₄N₅O₄S [M+H]⁺ m/z 650.24, found 650.15.

Step 2: (R)-3-(4,4-difluoroazepan-1-yl)-6-(4-(difluoromethyl)phenyl)-5-methyl-N-(3-(S-methylsulfonimidoyl)phenyl)pyridazine-4-carboxamide: A solution of tert-butyl (R)-((3-(3-(4,4-difluoroazepan-1-yl)-6-(4-(difluoromethyl)phenyl)-5-methylpyridazine-4-carboxamido)phenyl)(methyl)(oxo)-λ⁶-sulfaneylidene)carbamate (98 mg, 0.16 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (30 mL×3). The combined organic phases were washed with brine, dried over sodium sulfate, concentrated under vacuum. The residue was purified by prep-HPLC (Gemini 5 um C₁₈ column, 150*21.2 mm, eluting with 35% to 90% MeCN/H₂O containing 0.1% NH₃) to provide (R)-3-(4,4-difluoroazepan-1-yl)-6-(4-(difluoromethyl)phenyl)-5-methyl-N-(3-(S-methylsulfonimidoyl)phenyl)pyridazine-4-carboxamide (35.8 mg, 40.7% y) as a white solid. ¹H NMR (400 MHz, DMSO-d₆) δ 11.11 (s, 1H), 8.33 (s, 1H), 7.90 (d, J=8.4 Hz, 1H), 7.74-7.69 (m, 5H), 7.63-7.59 (m, 1H), 7.13 (t, J=5.6 Hz, 1H), 4.25 (s, 1H), 3.81-3.79 (m, 2H), 3.69-3.66 (m, 2H), 3.07 (s, 3H), 2.39-2.33 (m, 2H), 2.21 (s, 3H), 2.07-2.01 (m, 2H), 1.89-1.88 (m, 2H). LCMS (ESI) calcd. for C₂₆H₂₈F₄N₅O₂S [M+H]⁺ m/z 550.19, found 550.15.

### Example 168

### (R)-2-(4,4-difluoroazepan-1-yl)-5-fluoro-4-methyl-N-(3-(S-methylsulfonimidoyl)phenyl)nicotinamide

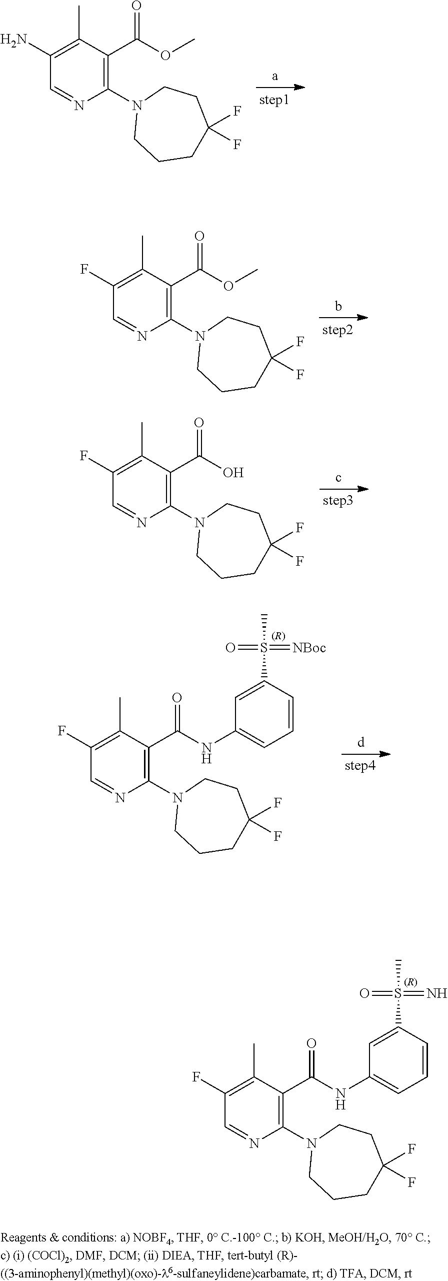

Step 1: methyl 2-(4,4-difluoroazepan-1-yl)-5-fluoro-4-methylnicotinate: To a solution of methyl 5-amino-2-(4,4-difluoroazepan-1-yl)-4-methylnicotinate (600 mg, 2.0 mmol) in THE (10 mL) was added nitrosyl tetrafluoroborate (282 mg, 2.4 mmol) at 0° C. Then the mixture was stirred at 0° C. for 1 hour. The resulting brown precipitate was collected by filtration and washed with THF. Then the filter cake was heated to 150° C. for 0.5 h. After the reaction was completed, the mixture was cooled to room temperature. The residue was purified by flash column chromatography on silica gel (PE/EtOAc=3/1) to give methyl 2-(4,4-difluoroazepan-1-yl)-5-fluoro-4-methylnicotinate (150 mg, 24.7%) as a yellow solid. LCMS (ESI) calcd. for C₁₄H₁₈F₃N₂O₂ [M+H]⁺ m/z 303.13, found 302.75.

Step 2: 2-(4,4-difluoroazepan-1-yl)-5-fluoro-4-methylnicotinic acid: A solution of methyl 2-(4,4-difluoroazepan-1-yl)-5-fluoro-4-methylnicotinate (150 mg, 0.5 mmol) and KOH (280 mg, 5 mmol) in MeOH/H₂O (1/1, 4 mL) was heated at 70° C. for 16 hours. After the reaction was completed, the mixture was cooled to room temperature. The residue was adjusted to pH=3-4 with aqueous HCl (1M). Then the solution was extracted with EtOAc (80 mL×3). The combined organic layers were washed with brine, dried over Na₂SO₄, concentrated under vacuum to give crude 2-(4,4-difluoroazepan-1-yl)-5-fluoro-4-methylnicotinic acid (110 mg) as a yellow oil. LCMS (ESI) calcd. for C₁₃H₁₆F₃N₂O₂ [M+H]⁺ m/z 289.12, found 288.75.

Step 3: tert-butyl (R)-((3-(2-(4,4-difluoroazepan-1-yl)-5-fluoro-4-methylnicotinamido)phenyl)(methyl)(oxo)-λ⁶-sulfaneylidene)carbamate: To a solution of 2-(4,4-difluoroazepan-1-yl)-5-fluoro-4-methylnicotinic acid (100 mg, 0.35 mmol) in DCM (5 mL) was added oxalyl chloride (80 mg, 0.62 mmol) and DMF (0.02 mL). The mixture was stirred at room temperature for 1 h. Then the mixture was concentrated under vacuum. The residue was diluted with THE (3 mL) and added to a solution of tert-butyl (R)-((3-aminophenyl)(methyl)(oxo)-λ⁶-sulfaneylidene)carbamate (104 mg, 0.38 mmol) and DIEA (135 mg, 1.04 mmol) in THE (3 mL). The mixture was heated at 50° C. for 1 hour. After the reaction was completed, the mixture was cooled to room temperature. The resulting solution was concentrated under vacuum. The residue was purified by flash column chromatography on silica gel (PE/EtOAc=1/1) to give tert-butyl (R)-((3-(2-(4,4-difluoroazepan-1-yl)-5-fluoro-4-methylnicotinamido)phenyl)(methyl)(oxo)-λ⁶-sulfaneylidene)carbamate (80 mg, 42.8%) as a yellow solid. LCMS (ESI) calcd. for C₂₅H₃₂F₃N₄O₄S [M+H]⁺ m/z 541.21, found 541.15.

Step 4: (R)-2-(4,4-difluoroazepan-1-yl)-5-fluoro-4-methyl-N-(3-(S-methylsulfonimidoyl)phenyl)nicotinamide: A solution of tert-butyl (R)-((3-(2-(4,4-difluoroazepan-1-yl)-5-fluoro-4-methylnicotinamido)phenyl)(methyl)(oxo)-λ⁶-sulfaneylidene)carbamate (80 mg, 0.15 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (30 mL×3). The combined organic layers were washed with brine, dried over sodium sulfate, concentrated under vacuum. The residue was purified by prep-HPLC (Gemini 5 um C₁₈ column, 150*21.2 mm, eluting with 30% to 80% MeCN/H₂O containing 0.05% NH₃) to provide (R)-2-(4,4-difluoroazepan-1-yl)-5-fluoro-4-methyl-N-(3-(S-methylsulfonimidoyl)phenyl)nicotinamide (32.5 mg, 49.9%) as a white solid. ¹H NMR (400 MHz, DMSO-d₆) δ 10.86 (s, 1H), 8.33 (t, J=1.8 Hz, 1H), 8.18 (s, 1H), 7.93-7.86 (m, 1H), 7.68 (d, J=8.0 Hz, 1H), 7.59 (t, J=7.9 Hz, 1H), 4.23 (s, 1H), 3.58-3.52 (m, 2H), 3.48 (t, J=6.1 Hz, 2H), 3.06 (s, 3H), 2.29-2.14 (m, 5H), 2.05-1.92 (m, 2H), 1.83-1.71 (m, 2H). LCMS (ESI) calcd. for C₂₀H₂₄F₃N₄O₂S [M+H]⁺ m/z 441.16, found 441.10.

### Intermediate 2

### tert-butyl (R)-((3-(5-bromo-2-(4,4-difluoroazepan-1-yl)-4-methyl-6-(trifluoromethyl)nicotinamido)phenyl)(methyl)(oxo)-λ⁶--sulfaneylidene)carbamate

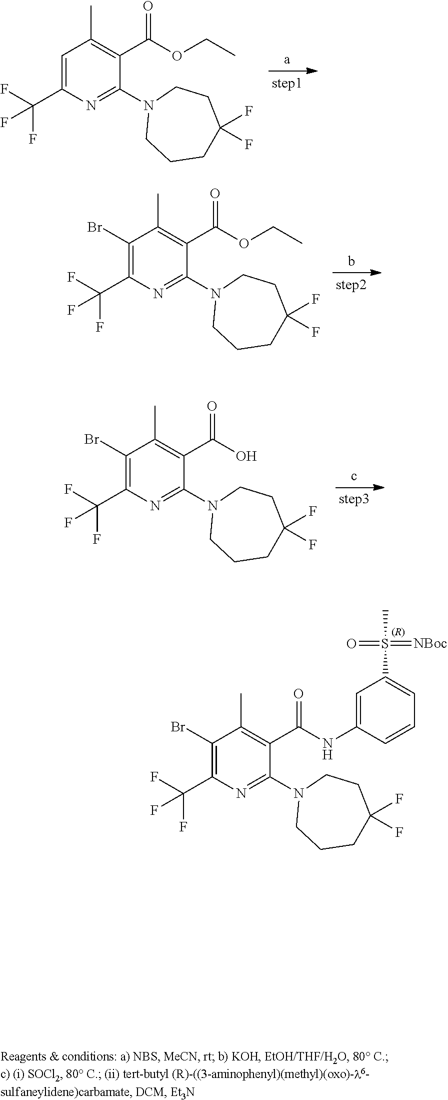

Step 1: ethyl 5-bromo-2-(4,4-difluoroazepan-1-yl)-4-methyl-6-(trifluoromethyl)nicotinate: A solution of ethyl 2-(4,4-difluoroazepan-1-yl)-4-methyl-6-(trifluoromethyl)nicotinate (1.4 g, 3.82 mmol) and NBS (817 mg, 4.59 mmol) in MeCN (30 mL) was stirred at room temperature for 2 hours. LCMS showed the reaction was completed. The mixture was diluted with water (60 mL) and extracted with EtOAc (50 mL×3). The combined organic phases were washed with brine, dried over sodium sulfate, concentrated under vacuum. The residue was purified by flash column chromatography on silica gel (PE/EtOAc=5/1) to provide ethyl 5-bromo-2-(4,4-difluoroazepan-1-yl)-4-methyl-6-(trifluoromethyl)nicotinate (1.6 g, 94.1%) as yellow solid. LCMS (ESI) calcd. for C₁₆H₁₉BrF₅N₂O₂ [M+H]⁺ m/z 447.06, found 447.05.

Step 2: 5-bromo-2-(4,4-difluoroazepan-1-yl)-4-methyl-6-(trifluoromethyl)nicotinic acid: To a solution of ethyl 5-bromo-2-(4,4-difluoroazepan-1-yl)-4-methyl-6-(trifluoromethyl)nicotinate (1.6 g, 3.59 mmol) in EtOH/THF/H₂O (1/1/1, 30 mL) was added KOH (2.01 g, 35.9 mmol) at 25° C. The mixture was heated at 80° C. for 16 hours. After the reaction was completed, the mixture was concentrated to remove most EtOH/THF. The aqueous phase was adjusted to pH=3-4 with 1N HCl then extracted with EtOAc (50 mL×3). The combined organic phases were washed with brine, dried with Na₂SO₄, and concentrated under reduced pressure to afford 5-bromo-2-(4,4-difluoroazepan-1-yl)-4-methyl-6-(trifluoromethyl)nicotinic acid (1.2 g, 80.1%) as a yellow solid. LCMS (ESI) calcd. for C₁₄H₁₅BrF₅N₂O₂ [M+H]⁺ m/z 417.03, found 417.00.

Step 3: tert-butyl (R)-((3-(5-bromo-2-(4,4-difluoroazepan-1-yl)-4-methyl-6-(trifluoromethyl)nicotinamido)phenyl)(methyl)(oxo)-λ⁶-sulfaneylidene)carbamate: A solution of 5-bromo-2-(4,4-difluoroazepan-1-yl)-4-methyl-6-(trifluoromethyl)nicotinic acid (600 mg, 1.44 mmol) in SOCl₂ (6 mL) was heated to 80° C. for 0.5 h. The solution was concentrated under vacuum to provide the chloride intermediate. Then the chloride intermediate dissolved in DCM (5 mL) was added to a solution of tert-butyl (R)-((3-aminophenyl)(methyl)(oxo)-λ⁶-sulfaneylidene)carbamate (465 mg, 1.72 mmol) and TEA (436 mg, 4.30 mmol) in DCM (6 mL) at 0° C. The resulting mixture was stirred at 40° C. for 2 hours. Then the mixture was quenched with water (50 mL) and extracted with DCM (50 mL×3). The combined organic phases were washed with brine, dried over sodium sulfate, concentrated under vacuum. The residue was purified by flash column chromatography on silica gel (PE/EtOAc=1/1) to provide tert-butyl (R)-((3-(5-bromo-2-(4,4-difluoroazepan-1-yl)-4-methyl-6-(trifluoromethyl)nicotinamido)phenyl)(methyl)(oxo)-λ⁶-sulfaneylidene)carbamate (450 mg, 32.7%) as a yellow solid. LCMS (ESI) calcd. for C₂₆H₃₁BrF₅N₄O₄S [M+H]⁺ m/z 671.12, found 671.05.

### Example 169

### (R)-2-(4,4-difluoroazepan-1-yl)-4-methyl-N-(3-(S-methylsulfonimidoyl)phenyl)-5-(4-(trifluoromethyl)phenyl)nicotinamide

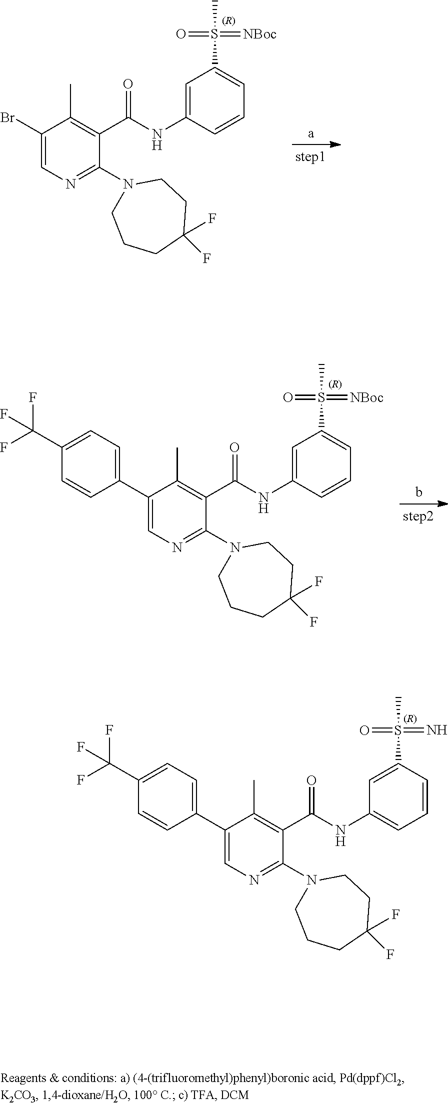

Step 1: tert-butyl (R)-((3-(2-(4,4-difluoroazepan-1-yl)-4-methyl-5-(4-(trifluoromethyl)phenyl)nicotinamido)phenyl)(methyl)(oxo)-λ⁶-sulfaneylidene)carbamate: A mixture of tert-butyl (R)-((3-(5-bromo-2-(4,4-difluoroazepan-1-yl)-4-methylnicotinamido)phenyl)(methyl)(oxo)-λ⁶-sulfaneylidene)carbamate (100 mg, 0.17 mmol), (4-(trifluoromethyl)phenyl)boronic acid (63.1 mg, 0.33 mmol), potassium carbonate (162.5 mg, 0.5 mmol) and [1,1′-Bis(diphenylphosphino)ferrocene]dichloropalladium (II) (24.3 mg, 0.03 mmol) in 1,4-dioxane/H₂O (4/1, 10 mL) was heated at 100° C. for 5 h under an atmosphere of N₂. After the reaction was completed, the mixture was cooled to room temperature. The resulting solution was diluted with water (30 mL) and extracted with EtOAc (50 mL×3). The combine organic phases were washed with brine, dried over Na₂SO₄, concentrated under vacuum. The residue was purified by flash column chromatography on silica gel (PE/EtAOc=1/1) to give tert-butyl (R)-((3-(2-(4,4-difluoroazepan-1-yl)-4-methyl-5-(4-(trifluoromethyl)phenyl)nicotinamido)phenyl)(methyl)(oxo)-λ⁶-sulfaneylidene)carbamate 2 (96 mg, 86.6%) as a yellow oil. LCMS (ESI) calcd. for C₃₂H₃₆F₅N₄O₄S [M+H]⁺ m/z 667.24, found 667.20.

Step 2: (R)-2-(4,4-difluoroazepan-1-yl)-4-methyl-N-(3-(S-methylsulfonimidoyl)phenyl)-5-(4-(trifluoromethyl)phenyl)nicotinamide: A solution of give tert-butyl (R)-((3-(2-(4,4-difluoroazepan-1-yl)-4-methyl-5-(4-(trifluoromethyl)phenyl)nicotinamido)phenyl)(methyl)(oxo)-λ⁶-sulfaneylidene)carbamate (96 mg, 0.15 mmol) in DCM (5 mL) was added TFA (1 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 40% to 90% MeCN/H₂O containing 0.05% NH₃) to provide (R)-2-(4,4-difluoroazepan-1-yl)-4-methyl-N-(3-(S-methylsulfonimidoyl)phenyl)-5-(4-(trifluoromethyl)phenyl)nicotinamide (45.3 mg, 55.5%) as a white solid. ¹H NMR (400 MHz, DMSO-d₆) δ 10.90 (s, 1H), 8.37 (s, 1H), 8.13 (s, 1H), 7.91 (d, J=8.0 Hz, 1H), 7.85-7.79 (m, 2H), 7.67 (d, J=7.9 Hz, 1H), 7.63-7.56 (m, 3H), 4.23 (s, 1H), 3.71-3.65 (m, 2H), 3.60 (t, J=6.0 Hz, 2H), 3.06 (s, 3H), 2.36-2.27 (m, 2H), 2.17 (s, 3H), 2.09-1.96 (m, 2H), 1.89-1.78 (m, 2H). LCMS (ESI) calcd. for C₂₇H₂₈F₅N₄O₂S [M+H]⁺ m/z 567.19, found 567.15.

### Example 170

### (R)-5,6-dicyano-2-(4,4-difluoroazepan-1-yl)-4-methyl-N-(3-(S-methylsulfonimidoyl)phenyl)nicotinamide

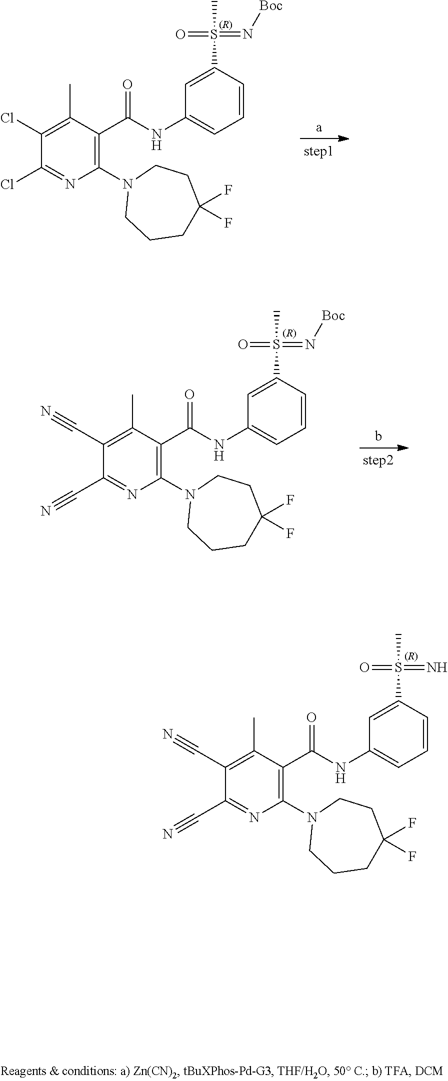

Step 1: tert-butyl (R)-((3-(5,6-dicyano-2-(4,4-difluoroazepan-1-yl)-4-methylnicotinamido)phenyl)(methyl)(oxo)-λ⁶-sulfaneylidene)carbamate: A solution of tert-butyl (R)-((3-(5,6-dichloro-2-(4,4-difluoroazepan-1-yl)-4-methylnicotinamido)phenyl)(methyl)(oxo)-λ⁶-sulfaneylidene)carbamate (100 mg, 0.17 mmol), Zn(CN)₂ (40 mg, 0.34 mmol) and tBuXPhos-Pd-G3 (27 mg, 0.03 mmol) in THE (8 mL) and H₂O (2 mL) was heated at 50° C. for 6 hours. LCMS showed the reaction was completed. The mixture was diluted with water (10 mL) and extracted with EtOAc (10 mL×3). The combined organic layers were washed with brine, dried over sodium sulfate, concentrated under vacuum. The residue was purified by flash column chromatography on silica gel (PE/EtOAc=1/1) to provide tert-butyl (R)-((3-(5,6-dicyano-2-(4,4-difluoroazepan-1-yl)-4-methylnicotinamido)phenyl)(methyl)(oxo)-λ⁶-sulfaneylidene)carbamate (60 mg, 61.88%) as a yellow solid. LCMS (ESI) calcd. for C₂₇H₃₀F₂N₆O₄SNa [M+H]⁺ m/z 595.19, found 595.15.

Step 2: (R)-5,6-dicyano-2-(4,4-difluoroazepan-1-yl)-4-methyl-N-(3-(S-methylsulfonimidoyl)phenyl)nicotinamide: A solution of tert-butyl (R)-((3-(5,6-dicyano-2-(4,4-difluoroazepan-1-yl)-4-methylnicotinamido)phenyl)(methyl)(oxo)-λ⁶-sulfaneylidene)carbamate (60 mg, 0.10 mmol) in DCM (2 mL) was added TFA (0.2 mL) at room temperature. The reaction mixture was stirred at room temperature for 1 hour. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.05% NH₃·H₂O) to provide (R)-5,6-dicyano-2-(4,4-difluoroazepan-1-yl)-4-methyl-N-(3-(S-methylsulfonimidoyl)phenyl)nicotinamide (24.2 mg, 48.85%) as a white solid. ¹H NMR (400 MHz, DMSO-d₆) δ 11.02 (s, 1H), 8.25 (s, 1H), 7.88 (d, J=7.9 Hz, 1H), 7.76-7.67 (m, 1H), 7.62 (t, J=7.9 Hz, 1H), 4.26 (s, 1H), 3.69 (d, J=45.8 Hz, 4H), 3.07 (s, 3H), 2.43 (s, 3H), 2.36-2.22 (m, 2H), 2.12-1.95 (m, 2H), 1.92-1.77 (m, 2H). LCMS (ESI) calcd. for C₂₂H₂₃F₂N₆O₂S [M+H]⁺ m/z 473.16, found 473.10.

### Example 171

### (R)-5-chloro-6-cyano-2-(4,4-difluoroazepan-1-yl)-4-methyl-N-(3-(S-methylsulfonimidoyl)phenyl)nicotinamide

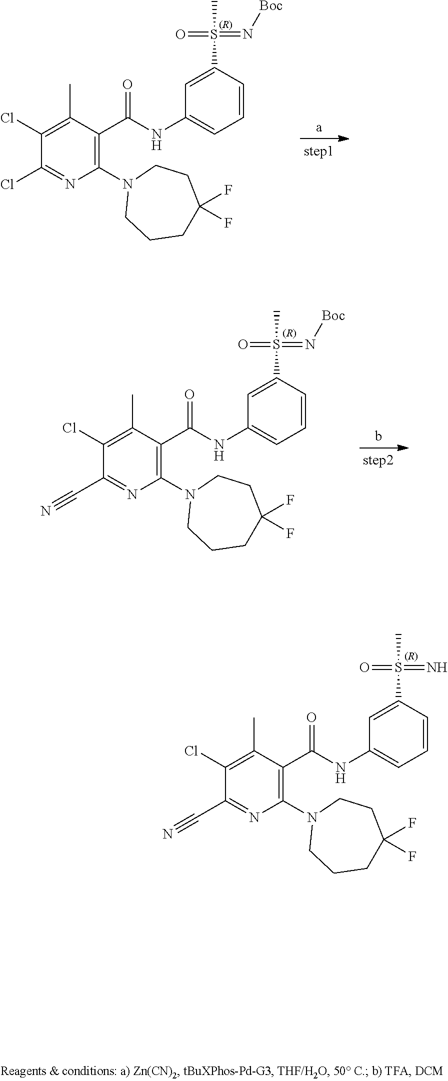

Step 1: tert-butyl (R)-((3-(5-chloro-6-cyano-2-(4,4-difluoroazepan-1-yl)-4-methylnicotinamido)phenyl)(methyl)(oxo)-λ⁶-sulfaneylidene)carbamate: A solution of tert-butyl (R)-((3-(5,6-dichloro-2-(4,4-difluoroazepan-1-yl)-4-methylnicotinamido)phenyl)(methyl)(oxo)-λ⁶-sulfaneylidene)carbamate (100 mg, 0.17 mmol), Zn(CN)₂ (20 mg, 0.17 mmol) and tBuXPhos-Pd-G3 (27 mg, 0.03 mmol) in THE (8 mL) and H₂O (2 mL) was heated at 50° C. for 6 hours. LCMS showed the reaction was completed. The mixture was diluted with water (10 mL) and extracted with EtOAc (10 mL×3). The combined organic layers were washed with brine, dried over sodium sulfate, concentrated under vacuum. The residue was purified by flash column chromatography on silica gel (PE/EtOAc=1/1) to provide tert-butyl (R)-((3-(5-chloro-6-cyano-2-(4,4-difluoroazepan-1-yl)-4-methylnicotinamido)phenyl)(methyl)(oxo)-λ⁶-sulfaneylidene)carbamate (20 mg, 20.33%) as a yellow solid. LCMS (ESI) calcd. for C₂₆H₃₀ClF₂N₅O₄SNa [M+Na]⁺ m/z 604.17, found 604.10.

Step 2: (R)-5-chloro-6-cyano-2-(4,4-difluoroazepan-1-yl)-4-methyl-N-(3-(S-methylsulfonimidoyl)phenyl)nicotinamide: A solution of tert-butyl (R)-((3-(5-chloro-6-cyano-2-(4,4-difluoroazepan-1-yl)-4-methylnicotinamido)phenyl)(methyl)(oxo)-λ⁶-sulfaneylidene)carbamate (20 mg, 0.03 mmol) in DCM (2 mL) was added TFA (0.2 mL) at room temperature. The reaction mixture was stirred at room temperature for 1 hour. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.05% NH₃·H₂O) to provide (R)-5-chloro-6-cyano-2-(4,4-difluoroazepan-1-yl)-4-methyl-N-(3-(S-methylsulfonimidoyl)phenyl)nicotinamide (5.2 mg, 30.32%) as a white solid. ¹H NMR (400 MHz, DMSO-d₆) δ 11.02 (s, 1H), 8.27 (d, J=1.7 Hz, 1H), 7.88 (d, J=9.0 Hz, 1H), 7.71 (d, J=8.2 Hz, 1H), 7.61 (t, J=7.9 Hz, 1H), 4.25 (s, 1H), 3.62 (d, J=5.1 Hz, 2H), 3.56 (t, J=6.0 Hz, 2H), 3.06 (d, J=0.6 Hz, 3H), 2.34 (s, 3H), 2.31-2.18 (m, 2H), 2.07-1.94 (m, 2H), 1.87-1.76 (m, 2H). LCMS (ESI) calcd. for C₂₁H₂₃ClF₂N₅O₂S [M+H]⁺ m/z 482.13, found 482.05.

### Example 172

### (R)-5-bromo-2-(4,4-difluoroazepan-1-yl)-4,6-dimethyl-N-(3-(S-methylsulfonimidoyl)phenyl)nicotinamide

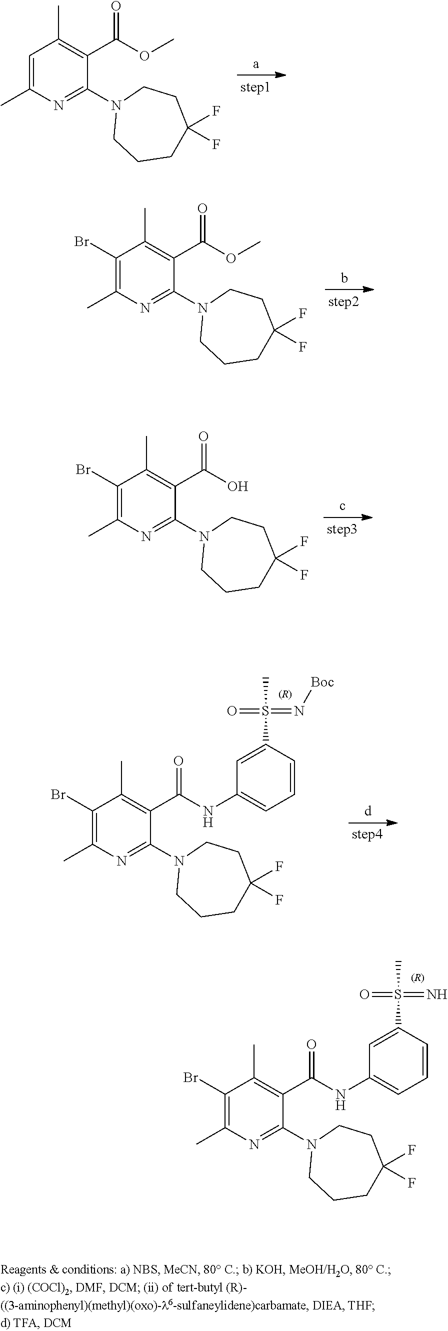

Step 1: methyl 5-bromo-2-(4,4-difluoroazepan-1-yl)-4,6-dimethylnicotinate: A solution of methyl 2-(4,4-difluoroazepan-1-yl)-4,6-dimethylnicotinate (700 mg, 2.35 mmol) and NBS (459 mg, 2.58 mmol) in MeCN (10 mL) was stirred heated at 80° C. for 2 hours. LCMS showed the reaction was completed. The mixture was diluted with water (30 mL) and extracted with EtOAc (30 mL×3). The combined organic phases were washed with brine, dried over sodium sulfate, concentrated under vacuum. The residue was purified by flash column chromatography on silica gel (PE/EtOAc=5/1) to provide methyl 5-bromo-2-(4,4-difluoroazepan-1-yl)-4,6-dimethylnicotinate (780 mg, 88.1%) as yellow solid. LCMS (ESI) calcd. for C₁₅H₂₀BrF₂N₂O₂ [M+H]⁺ m/z 377.07, found 377.00.

Step 2: 5-bromo-2-(4,4-difluoroazepan-1-yl)-4,6-dimethylnicotinic acid: To a solution of methyl 5-bromo-2-(4,4-difluoroazepan-1-yl)-4,6-dimethylnicotinate (780 mg, 2.07 mmol) in MeOH/H₂O (1/1, 10 mL) was added KOH (1.16 g, 20.68 mmol) at 25° C. The mixture was heated at 80° C. for 16 hours. After the reaction was completed, the mixture was concentrated to remove most MeOH. The aqueous phase was adjusted to pH=3-4 with 1N HCl then extracted with EtOAc (50 mL×3). The combined organic phases were washed with brine, dried with Na₂SO₄, and concentrated under reduced pressure to afford 5-bromo-2-(4,4-difluoroazepan-1-yl)-4,6-dimethylnicotinic acid (650 mg, 86.6%) as a yellow solid. LCMS (ESI) calcd. for C₁₄H₁₈BrF₂N₂O₂ [M+H]⁺ m/z 363.05, found 363.00.

Step 3: tert-butyl (R)-((3-(5-bromo-2-(4,4-difluoroazepan-1-yl)-4,6-dimethylnicotinamido)phenyl)(methyl)(oxo)-λ⁶-sulfaneylidene)carbamate: To a solution of 5-bromo-2-(4,4-difluoroazepan-1-yl)-4,6-dimethylnicotinic acid (650 mg, 1.79 mmol) in DCM (10 mL) was added oxalyl chloride (1.14 g, 8.95 mmol) and DMF (20 L). The mixture was stirred at room temperature for 0.5 hour. Then the mixture was concentrated under vacuum. The residue was dissolved in DCM (10 mL) and added to a solution of tert-butyl (R)-((3-aminophenyl)(methyl)(oxo)-λ⁶-sulfaneylidene)carbamate (583 mg, 2.15 mmol) and TEA (906 mg, 8.95 mmol) in DCM (10 mL). The mixture was stirred at room temperature for 1 hour. After the reaction was completed, the mixture was concentrated under vacuum. The residue was purified by flash column chromatography on silica gel (PE/EtOAc=2/1) to give tert-butyl (R)-((3-(5-bromo-2-(4,4-difluoroazepan-1-yl)-4,6-dimethylnicotinamido)phenyl)(methyl)(oxo)-λ⁶-sulfaneylidene)carbamate (570 mg, 51.7%) as a yellow oil. LCMS (ESI) calcd. for C₂₆H₃₄BrF₂N₄O₄S [M+H]⁺ m/z 615.15, found 615.10.

Step 4: (R)-5-bromo-2-(4,4-difluoroazepan-1-yl)-4,6-dimethyl-N-(3-(S-methylsulfonimidoyl)phenyl)nicotinamide: A solution of tert-butyl (R)-((3-(5-bromo-2-(4,4-difluoroazepan-1-yl)-4,6-dimethylnicotinamido)phenyl)(methyl)(oxo)-λ⁶-sulfaneylidene)carbamate (110 mg, 0.18 mmol) in DCM (5 mL) was added TFA (0.5 mL) at room temperature. The reaction mixture was stirred at room temperature for 2 hours. After the reaction was completed, the solution was diluted with water (10 mL) and extracted with DCM (10 mL×3). The combine organic phases were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.1% NH₄OH) to obtain (R)-5-bromo-2-(4,4-difluoroazepan-1-yl)-4,6-dimethyl-N-(3-(S-methylsulfonimidoyl)phenyl)nicotinamide (50.2 mg, 54.6%) as a white solid. ¹H NMR (400 MHz, DMSO-d₆) δ 10.82 (s, 1H), 8.32 (t, J=1.8 Hz, 1H), 7.91-7.83 (m, 1H), 7.69-7.54 (m, 2H), 4.23 (s, 1H), 3.63-3.45 (m, 4H), 3.05 (s, 3H), 2.51 (s, 3H), 2.34-2.19 (m, 5H), 2.04-1.91 (m, 2H), 1.82-1.73 (m, 2H). LCMS (ESI) calcd. for C₂₁H₂₆BrF₂N₄O₂S [M+H]⁺ m/z 517.09, found 517.05.

### Example 173

### 5-(azetidin-3-yl)-2-(4,4-difluoroazepan-1-yl)-4-methyl-N-(3-(S-methylsulfonimidoyl)phenyl)nicotinamide

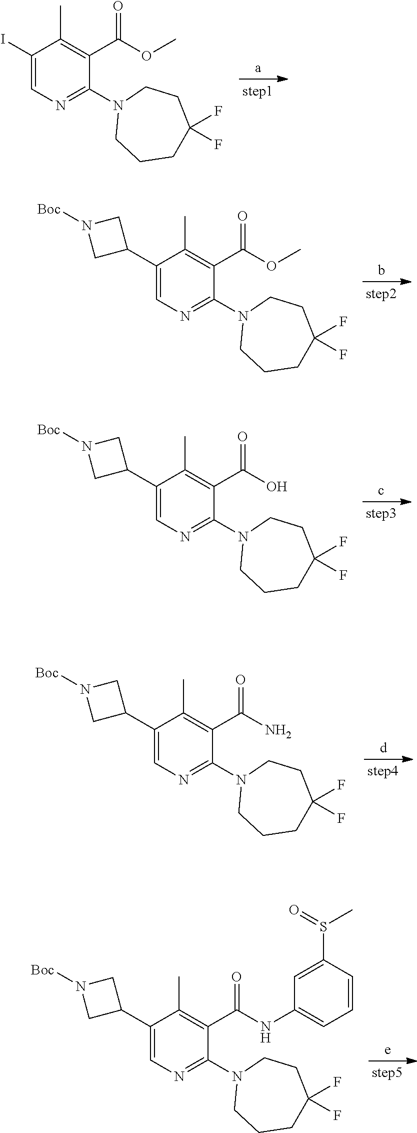 [SMILES: COC(=O)c1c(N2CCCC(F)(F)CC2)ncc(I)c1C ; COC(=O)c1c(N2CCCC(F)(F)CC2)ncc(C2CN(C(=O)OC(C)(C)C)C2)c1C ; Cc1c(C2CN(C(=O)OC(C)(C)C)C2)cnc(N2CCCC(F)(F)CC2)c1C(=O)O ; Cc1c(C2CN(C(=O)OC(C)(C)C)C2)cnc(N2CCCC(F)(F)CC2)c1C(N)=O ; Cc1c(C2CN(C(=O)OC(C)(C)C)C2)cnc(N2CCCC(F)(F)CC2)c1C(=O)Nc1cccc(S(C)=O)c1]

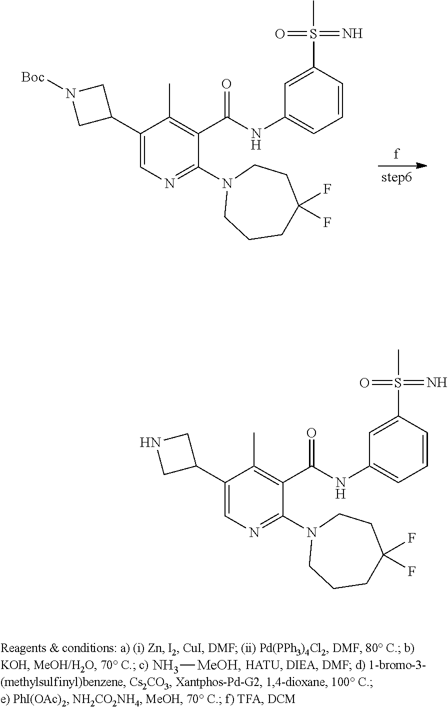

Step 1: methyl 5-(1-(tert-butoxycarbonyl)azetidin-3-yl)-2-(4,4-difluoroazepan-1-yl)-4-methylnicotinate: A mixture of Zn power (2.8 g, 0.044 mol) and 12 (250 mg, 0.984 mmol) in a 250 mL three-necked flask was evacuated and backfilled with N₂ three times and then charged with N₂. The flask was heated carefully with a hot air blower until 12 was sublimated. Then a solution of tert-butyl 3-iodoazetidine-1-carboxylate (1.5 g, 0.0053 mol) in DMF (5 mL) was added intermediately via a syringe. The mixture was stirred for 10 minutes at room temperature. Then the heater and stirrer were removed. The upper layer clear solution was added dropwise into a stirred solution of methyl 2-(4,4-difluoroazepan-1-yl)-5-iodo-4-methylnicotinate (1 g, 0.0024 mol), CuI (200 mg, 1.053 mmol) and Pd(pph₃)₂C₁₂ (200 mg, 0.284 mmol) in DMF (4 mL) under an atmosphere of N₂. The mixture was heated at 80° C. for 1 hour. After the reaction was completed, the mixture was cooled to room temperature and the mixture was diluted with water (20 mL) and extracted with DCM (20 mL×3). The combine organic phases were washed with brine, dried over sodium sulfate, concentrated under vacuum. The residue was purified by flash column chromatography on silica gel (PE/EtOAc=1/1) to give methyl 5-(1-(tert-butoxycarbonyl)azetidin-3-yl)-2-(4,4-difluoroazepan-1-yl)-4-methylnicotinate (700 mg, 70.38%) as a white oil. LCMS (ESI) calcd. for C₂₂H₃₂F₂N₃O₄ [M+H]⁺ m/z 440.24, found 440.10.

Step 2: 5-(1-(tert-butoxycarbonyl)azetidin-3-yl)-2-(4,4-difluoroazepan-1-yl)-4-methylnicotinic acid: To a solution of methyl 5-(1-(tert-butoxycarbonyl)azetidin-3-yl)-2-(4,4-difluoroazepan-1-yl)-4-methylnicotinate (700 mg, 1.59 mmol) in MeOH/H₂O (v:v=1:1, 20 mL) was added potassium hydroxide (892 mg, 15.90 mmol) at room temperature. The mixture was stirred for 10 hours at 80° C. The reaction was monitored by LCMS. After the reaction was completed, the mixture was cooled to room temperature, the reaction was extracted with DCM (20 mL), then the aqueous phase was adjusted to pH=3 with 1 N HCl and extracted with DCM (20 mL×3). The organic phase was dried with Na₂SO₄ and concentrated under reduced pressure to give 5-(1-(tert-butoxycarbonyl)azetidin-3-yl)-2-(4,4-difluoroazepan-1-yl)-4-methylnicotinic acid (550 mg, 81.24%) as a white solid. LCMS (ESI) calcd. for C₂₁H₃₀F₂N₃O₄ [M+H]⁺ m/z 426.22, found 426.10.

Step 3: tert-butyl 3-(5-carbamoyl-6-(4,4-difluoroazepan-1-yl)-4-methylpyridin-3-yl)azetidine-1-carboxylate: A solution of 5-(1-(tert-butoxycarbonyl)azetidin-3-yl)-2-(4,4-difluoroazepan-1-yl)-4-methylnicotinic acid (550 mg, 1.291 mmol) in DMF (20 mL) was added HATU (638 mg, 1.678 mmol), DIEA (534 mg, 4.131 mmol) and NH₃-MeOH (20 mL) at room temperature. The mixture was stirred at room temperature for 16 hours. After the reaction was completed, the mixture was cooled to room temperature and the mixture was diluted with water (20 mL) and extracted with DCM (20 mL×3). The combine organic phases were washed with brine, dried over sodium sulfate, concentrated under vacuum. The residue was purified by flash column chromatography on silica gel (PE/EtOAc=1/1) to give tert-butyl 3-(5-carbamoyl-6-(4,4-difluoroazepan-1-yl)-4-methylpyridin-3-yl)azetidine-1-carboxylate (480 mg, 87.59%) as a yellow solid. LCMS (ESI) calcd. for C₂₁H₃₁F₂N₄O₃ [M+H]⁺ m/z 425.24, found 425.10.

Step 4: tert-butyl 3-(6-(4,4-difluoroazepan-1-yl)-4-methyl-5-((3-(methylsulfinyl)phenyl)carbamoyl)pyridin-3-yl)azetidine-1-carboxylate: A solution of tert-butyl 3-(5-carbamoyl-6-(4,4-difluoroazepan-1-yl)-4-methylpyridin-3-yl)azetidine-1-carboxylate (480 mg, 1.129 mmol) in dioxane (10 mL) was added 1-bromo-3-(methylsulfinyl)benzene (749 mg, 3.388 mmol), cesium carbonate (956 mg, 2.935 mmol) and Xantphos-Pd-G2 (100 mg, 0.113 mmol) at room temperature. The reaction mixture was stirred at 100° C. for 16 hours under nitrogen. The reaction was monitored by LCMS. After the reaction was completed, the filtrate was diluted with water (20 mL) and extracted with DCM (20 mL×3). The combine organic phases were washed with brine, dried over sodium sulfate, concentrated under vacuum. The residue was purified by flash column chromatography on silica gel (PE/EtOAc=1/1) to give tert-butyl 3-(6-(4,4-difluoroazepan-1-yl)-4-methyl-5-((3-(methylsulfinyl)phenyl)carbamoyl)pyridin-3-yl)azetidine-1-carboxylate (250 mg, 39.37%) as a yellow oil. LCMS (ESI) calcd. for C₂₈H₃₇F₂N₄O₄S [M+H]⁺ m/z 563.25, found 563.15.

Step 5: tert-butyl 3-(6-(4,4-difluoroazepan-1-yl)-4-methyl-5-((3-(S-methylsulfonimidoyl)phenyl)carbamoyl)pyridin-3-yl)azetidine-1-carboxylate: To a solution of tert-butyl 3-(6-(4,4-difluoroazepan-1-yl)-4-methyl-5-((3-(methylsulfinyl)phenyl)carbamoyl)pyridin-3-yl)azetidine-1-carboxylate (250 mg, 0.444 mmol) in MeOH (10 mL) was added PhI(OAc)₂ (358 mg, 1.110 mmol) and ammonium carbamate (104 mg, 1.332 mmol) at room temperature. The reaction mixture was heated at 70° C. for 1 hour. After the reaction was completed, the mixture was cooled to room temperature, diluted with water (10 mL), and extracted with DCM (10 mL×3). The combine organic phases were washed with brine, dried over sodium sulfate, concentrated under vacuum. The residue was first purified by flash column chromatography on silica gel (PE/EtOAc=1/1), then further purified by prep-HPLC (Gemini 5 um C₁₈ column, 150*21.2 mm, eluting with 30% to 90% MeCN/H₂O containing 0.1% formic acid) to afford tert-butyl 3-(6-(4,4-difluoroazepan-1-yl)-4-methyl-5-((3-(S-methylsulfonimidoyl)phenyl)carbamoyl)pyridin-3-yl)azetidine-1-carboxylate (70 mg, 27.34%) as a white oil. LCMS (ESI) calcd. for C₂₈H₃₈F₂N₅O₄S [M+H]⁺ m/z 578.26, found 578.20.

Step 6: 5-(azetidin-3-yl)-2-(4,4-difluoroazepan-1-yl)-4-methyl-N-(3-(S-methylsulfonimidoyl)phenyl)nicotinamide: A solution of tert-butyl 3-(6-(4,4-difluoroazepan-1-yl)-4-methyl-5-((3-(S-methylsulfonimidoyl)phenyl)carbamoyl)pyridin-3-yl)azetidine-1-carboxylate (70 mg, 0.121 mmol) in DCM (5 mL) was added TFA (0.5 mL) at room temperature. The reaction mixture was stirred at room temperature for 2 hours. After the reaction was completed, the solution was diluted with saturated aqueous NaHCO₃ (10 mL) and extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.1% NH₃) to afford 5-(azetidin-3-yl)-2-(4,4-difluoroazepan-1-yl)-4-methyl-N-(3-(S-methylsulfonimidoyl)phenyl)nicotinamide (4.20 mg, 7.17%) as a white solid. ¹H NMR (400 MHz, DMSO-d₆) δ 10.77 (s, 1H), 8.34 (s, 1H), 8.10 (s, 1H), 7.88 (d, J=8.8 Hz, 1H), 7.66 (d, J=7.6 Hz, 1H), 7.59-7.55 (m, 1H), 4.21 (s, 1H), 3.94-3.92 (m, 1H), 3.71-3.67 (m, 4H), 3.55-3.49 (m, 2H), 3.36-3.35 (m, 2H), 3.05 (s, 3H), 2.23-2.05 (m, 2H), 2.06 (s, 3H), 1.99-1.91 (m, 2H), 178-1.76 (m, 2H). LCMS (ESI) calcd. for C₂₃H₃₀F₂N₅O₂S [M+H]⁺ m/z 478.21, found 478.05.

### Example 174

### N-(3-((R)-N-(2-amino-3,3,3-trifluoropropanoyl)-S-methylsulfonimidoyl)phenyl)-2-(4,4-difluoroazepan-1-yl)-4-methyl-5-(1-methyl-1H-pyrazol-4-yl)nicotinamide

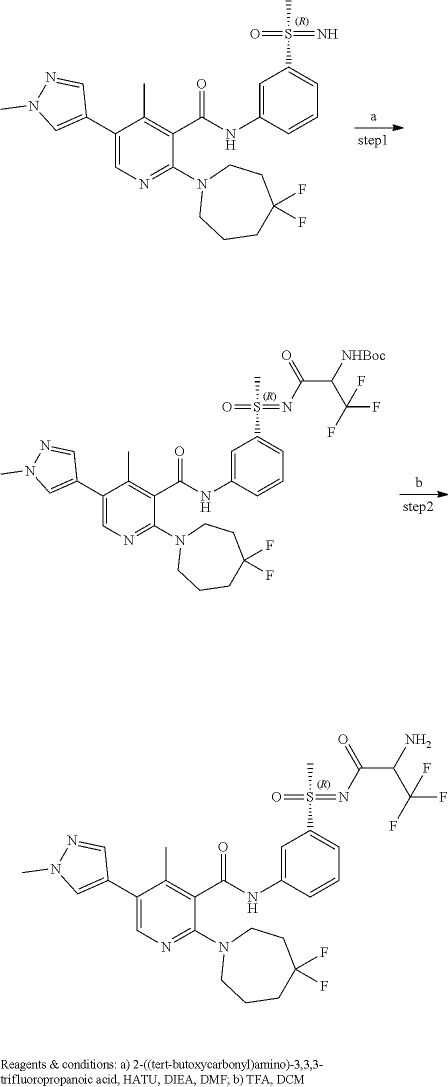

Step 1: tert-butyl (3-(((R)-(3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)amino)-1,1,1-trifluoro-3-oxopropan-2-yl)carbamate: To a solution of (R)-2-(4,4-difluoroazepan-1-yl)-4-methyl-5-(1-methyl-1H-pyrazol-4-yl)-N-(3-(S-methylsulfonimidoyl)phenyl)nicotinamide (150 mg, 0.30 mmol), 2-((tert-butoxycarbonyl)amino)-3,3,3-trifluoropropanoic acid (109 mg, 0.45 mmol) and TCFH (148 mg, 0.61 mmol) in MeCN (5 mL) and was added NMI (50 mg, 0.61 mmol). Then the mixture was stirred at 25° C. for 3 hours. LCMS showed the reaction was completed. The mixture was concentrated under vacuum. The residue was purified by flash column chromatography on silica gel (PE/EtOAc=1/4) to provide tert-butyl (3-(((R)-(3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)amino)-1,1,1-trifluoro-3-oxopropan-2-yl)carbamate (130 mg, 59.91%) as a yellow oil. LCMS (ESI) calcd. for C₃₂H₃₉F₅N₇O₅S [M+H]⁺ m/z 728.27, found 728.25.

Step 2: N-(3-((R)-N-(2-amino-3,3,3-trifluoropropanoyl)-S-methylsulfonimidoyl)phenyl)-2-(4,4-difluoroazepan-1-yl)-4-methyl-5-(1-methyl-1H-pyrazol-4-yl)nicotinamide: To a solution of tert-butyl (3-(((R)-(3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)amino)-1,1,1-trifluoro-3-oxopropan-2-yl)carbamate (130 mg, 0.18 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (10 mL×3). The combined organic phases were washed with brine, dried over sodium sulfate, concentrated under vacuum. The residue was purified by prep-HPLC (Gemini 5 um C₁₈ column, 150*21.2 mm, eluting with 30% to 90% MeCN/H₂O containing 0.05% NH₃) to provide N-(3-((R)-N-(2-amino-3,3,3-trifluoropropanoyl)-S-methylsulfonimidoyl)phenyl)-2-(4,4-difluoroazepan-1-yl)-4-methyl-5-(1-methyl-1H-pyrazol-4-yl)nicotinamide (68 mg, 60.71%) as a white solid. ¹H NMR (400 MHz, DMSO-d₆) δ 10.96 (d, J=1.4 Hz, 1H), 8.43 (d, J=1.6 Hz, 1H), 8.18 (s, 1H), 8.02-7.94 (m, 1H), 7.90 (s, 1H), 7.71-7.64 (m, 2H), 7.61 (s, 1H), 4.00-3.91 (m, 1H), 3.89 (s, 3H), 3.64-3.57 (m, 2H), 3.57-3.49 (m, 5H), 2.34-2.16 (m, 7H), 2.06-1.93 (m, 2H), 1.85-1.72 (m, 2H). LCMS (ESI) calcd. for C₂₇H₃₁F₅N₇O₃S [M+H]⁺ m/z 628.22, found 628.10.

### Example 175

### (R)-6-(4,4-difluoroazepan-1-yl)-2-methyl-3-(1-methyl-1H-pyrazol-4-yl)-N-(3-(S-methyl-N-(methylglycyl)sulfonimidoyl)phenyl)benzamide

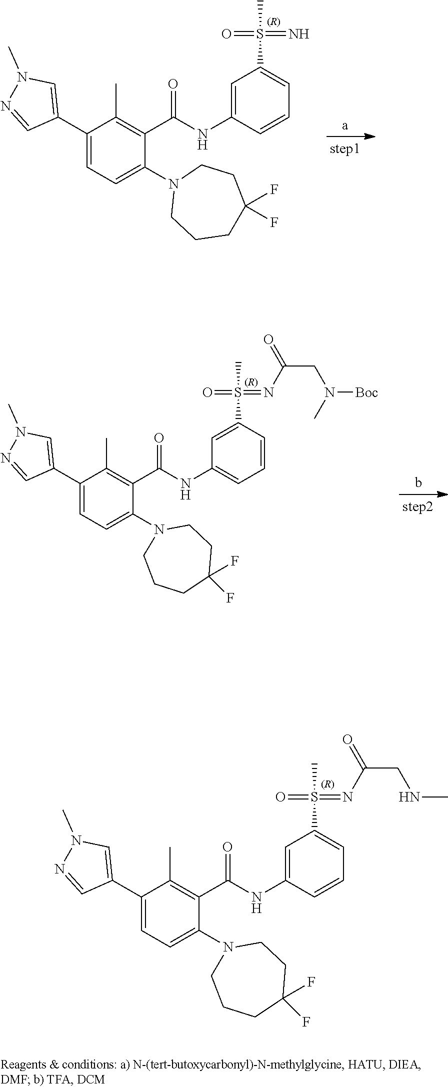

Step 1: tert-butyl (R)-(2-(((3-(6-(4,4-difluoroazepan-1-yl)-2-methyl-3-(1-methyl-1H-pyrazol-4-yl)benzamido)phenyl)(methyl)(oxo)-λ⁶-sulfaneylidene)amino)-2-oxoethyl)(methyl)carbamate: To a solution of (R)-6-(4,4-difluoroazepan-1-yl)-2-methyl-3-(1-methyl-1H-pyrazol-4-yl)-N-(3-(S-methylsulfonimidoyl)phenyl)benzamide (70 mg, 0.14 mmol), N-(tert-butoxycarbonyl)-N-methylglycine (32 mg, 0.17 mmol) and HATU (106 mg, 0.28 mmol) in DMF (3 mL) and was added DIEA (54 mg, 0.42 mmol). Then the mixture was stirred at 25° C. for 5 hours. LCMS showed the reaction was completed. The mixture was diluted with water and extracted with EtOAc (3×6 mL). The combined organic layers were washed with brine, dried with Na₂SO₄, and concentrated under vacuum. The residue was purified by silica gel column chromatography (eluting with PE/EtOAc, 40% to 70%) to give tert-butyl (R)-(2-(((3-(6-(4,4-difluoroazepan-1-yl)-2-methyl-3-(1-methyl-1H-pyrazol-4-yl)benzamido)phenyl)(methyl)(oxo)-λ⁶-sulfaneylidene)amino)-2-oxoethyl)(methyl)carbamate (60 mg, 80.2%) as a yellow oil. LCMS (ESI) calcd. for C₃₃H₄₃F₂N₆O₅S [M+H]⁺ m/z 673.30, found 673.20.

Step 2: (R)-6-(4,4-difluoroazepan-1-yl)-2-methyl-3-(1-methyl-1H-pyrazol-4-yl)-N-(3-(S-methyl-N-(methylglycyl)sulfonimidoyl)phenyl)benzamide: A solution of tert-butyl (R)-(2-(((3-(6-(4,4-difluoroazepan-1-yl)-2-methyl-3-(1-methyl-1H-pyrazol-4-yl)benzamido)phenyl)(methyl)(oxo)-λ⁶-sulfaneylidene)amino)-2-oxoethyl)(methyl)carbamate (60 mg, 0.09 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (10 mL×3). The combined organic layers were washed with brine, dried over sodium sulfate, concentrated under vacuum. The residue was purified by prep-HPLC (Gemini 5 um C₁₈ column, 150*21.2 mm, eluting with 40% to 90% MeCN/H₂O containing 0.1% NH₄OH) to obtain (R)-6-(4,4-difluoroazepan-1-yl)-2-methyl-3-(1-methyl-1H-pyrazol-4-yl)-N-(3-(S-methyl-N-(methylglycyl)sulfonimidoyl)phenyl)benzamide (31.3 mg, 61.5%) as a white solid. ¹H NMR (400 MHz, DMSO-d₆) δ 10.76 (s, 1H), 8.44 (s, 1H), 7.98 (dt, J=5.2, 2.7 Hz, 1H), 7.90 (s, 1H), 7.67-7.61 (m, 2H), 7.59 (s, 1H), 7.36 (d, J=8.4 Hz, 1H), 7.15 (d, J=8.4 Hz, 1H), 3.88 (s, 3H), 3.42 (s, 3H), 3.20 (s, 2H), 3.16-3.09 (m, 4H), 2.30-2.20 (m, 6H), 2.12-1.88 (m, 4H), 1.71-1.62 (m, 2H). LCMS (ESI) calcd. for C₂₈H₃₅F₂N₆O₃S [M+H]⁺ m/z 573.25, found 573.05.

### Example 176

### (R)-2-(4,4-difluoroazepan-1-yl)-4-methyl-N-(3-(S-methylsulfonimidoyl)phenyl)-5-(1-(trifluoromethyl)-1H-pyrazol-4-yl)nicotinamide

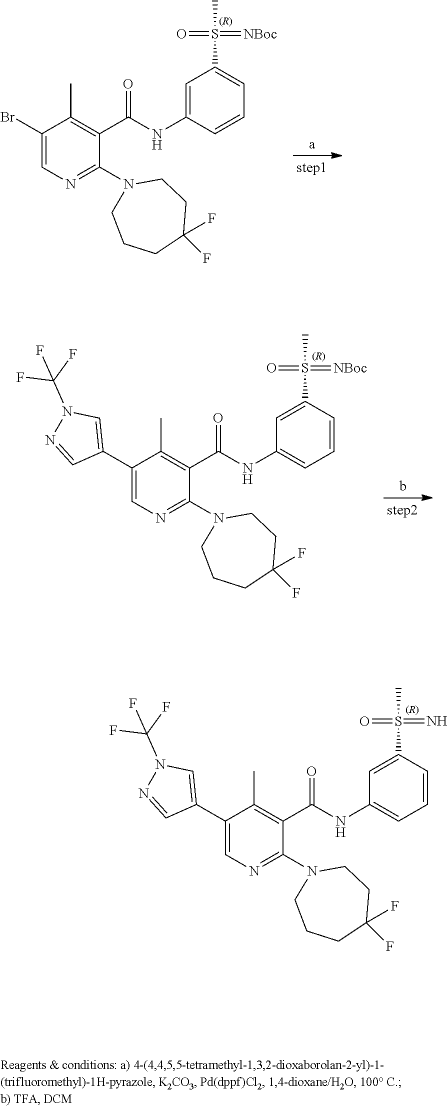

Step 1: tert-butyl (R)-((3-(2-(4,4-difluoroazepan-1-yl)-4-methyl-5-(1-(trifluoromethyl)-1H-pyrazol-4-yl)nicotinamido)phenyl)(methyl)(oxo)-λ⁶-sulfaneylidene)carbamate: A mixture of tert-butyl (R)-((3-(5-bromo-2-(4,4-difluoroazepan-1-yl)-4-methylnicotinamido)phenyl)(methyl)(oxo)-λ⁶-sulfaneylidene)carbamate (100 mg, 0.17 mmol), 4-(4,4,5,5-tetramethyl-1,3,2-dioxaborolan-2-yl)-1-(trifluoromethyl)-1H-pyrazole (87 mg, 0.33 mmol), K₂CO₃ (46 mg, 0.33 mmol) and Pd(dppf)Cl₂ (12 mg, 0.02 mmol) in dioxane (8 mL) and H₂O (2 mL) was heated at 100° C. for 16 hours under an atmosphere of N₂. After the reaction was completed, the mixture was cooled to room temperature. The resulting solution was diluted with water (40 mL) and extracted with EtOAc (40 mL×3). The combined organic phases were washed with brine, dried over Na₂SO₄, concentrated under vacuum. The residue was purified by flash column chromatography on silica gel (PE/EtOAc=2/1) to give tert-butyl (R)-((3-(2-(4,4-difluoroazepan-1-yl)-4-methyl-5-(1-(trifluoromethyl)-1H-pyrazol-4-yl)nicotinamido)phenyl)(methyl)(oxo)-λ⁶-sulfaneylidene)carbamate (50 mg, 47.5%) as a yellow solid. LCMS (ESI) calcd. for C₂₉H₃₄F₅N₆O₄S [M+H]⁺ m/z 657.23, found 657.25.

Step 2: (R)-2-(4,4-difluoroazepan-1-yl)-4-methyl-N-(3-(S-methylsulfonimidoyl)phenyl)-5-(1-(trifluoromethyl)-1H-pyrazol-4-yl)nicotinamide. A solution of tert-butyl (R)-((3-(2-(4,4-difluoroazepan-1-yl)-4-methyl-5-(1-(trifluoromethyl)-1H-pyrazol-4-yl)nicotinamido)phenyl)(methyl)(oxo)-λ⁶-sulfaneylidene)carbamate (50 mg, 0.08 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (30 mL×3). The combined organic layers were washed with brine, dried over sodium sulfate, concentrated under vacuum. The residue was purified by prep-HPLC (Gemini 5 um C₁₈ column, 150*21.2 mm, eluting with 25% to 75% MeCN/H₂O containing 0.1% NH₃) to provide (R)-2-(4,4-difluoroazepan-1-yl)-4-methyl-N-(3-(S-methylsulfonimidoyl)phenyl)-5-(1-(trifluoromethyl)-1H-pyrazol-4-yl)nicotinamide (15.30 mg, 34.7%) as a white solid. ¹H NMR (400 MHz, DMSO-d₆) δ 10.89 (s, 1H), 8.69 (s, 1H), 8.42-8.16 (m, 3H), 7.90 (d, J=8.1 Hz, 1H), 7.67 (d, J=8.1 Hz, 1H), 7.59 (t, J=7.9 Hz, 1H), 4.23 (s, 1H), 3.72-3.55 (m, 4H), 3.06 (s, 3H), 2.31-2.21 (m, 5H), 2.06-1.95 (m, 2H), 1.85-1.76 (m, 2H). LCMS (ESI) calcd. for C₂₄H₂₆F₅N₆O₂S [M+H]⁺ m/z 557.18, found 557.05.

### Example 177

### (R)-3-(4,4-difluoroazepan-1-yl)-N-(3-(N-glycyl-S-methylsulfonimidoyl)phenyl)-5-methyl-6-(trifluoromethyl)pyridazine-4-carboxamide

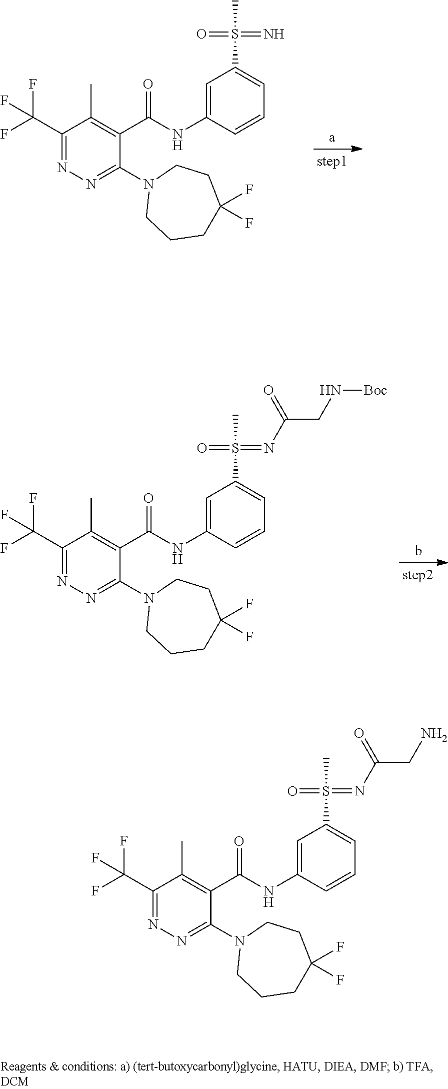 [SMILES: Cc1c(C(F)(F)F)nnc(N2CCCC(F)(F)CC2)c1C(=O)Nc1cccc([S@](C)(=N)=O)c1 ; Cc1c(C(F)(F)F)nnc(N2CCCC(F)(F)CC2)c1C(=O)Nc1cccc([S@@](C)(=O)=NC(=O)CNC(=O)OC(C)(C)C)c1 ; Cc1c(C(F)(F)F)nnc(N2CCCC(F)(F)CC2)c1C(=O)Nc1cccc([S@@](C)(=O)=NC(=O)CN)c1]

Step 1: tert-butyl (R)-(2-(((3-(3-(4,4-difluoroazepan-1-yl)-5-methyl-6-(trifluoromethyl)pyridazine-4-carboxamido)phenyl)(methyl)(oxo)-λ⁶-sulfaneylidene)amino)-2-oxoethyl)carbamate: A mixture of (R)-3-(4,4-difluoroazepan-1-yl)-5-methyl-N-(3-(S-methylsulfonimidoyl)phenyl)-6-(trifluoromethyl)pyridazine-4-carboxamide (80 mg, 0.16 mmol) and (tert-butoxycarbonyl)glycine (57 mg, 0.32 mmol) in DMF (5 mL) was added HATU (91 mg, 0.24 mmol) and DIEA (62 mg, 0.48 mmol). The mixture was stirred at room temperature for 16 hours. The reaction was monitored by LCMS. After the reaction was completed, the resulting solution was diluted with water (20 mL) and extracted with DCM (20 mL×3). The combined organic layers were washed with brine, dried over Na₂SO₄, concentrated under vacuum. The residue was purified by flash column chromatography on silica gel (PE/EtOAc=2/1 to 1/1) to give tert-butyl (R)-(2-(((3-(3-(4,4-difluoroazepan-1-yl)-5-methyl-6-(trifluoromethyl)pyridazine-4-carboxamido)phenyl)(methyl)(oxo)-λ⁶-sulfaneylidene)amino)-2-oxoethyl)carbamate (70 mg, 67.4%) as a yellow solid. LCMS (ESI) calcd. for C₂₇H₃₄F₅N₆O₅S [M+H]⁺ m/z 649.23, found 649.20.

Step 2: (R)-3-(4,4-difluoroazepan-1-yl)-N-(3-(N-glycyl-S-methylsulfonimidoyl)phenyl)-5-methyl-6-(trifluoromethyl)pyridazine-4-carboxamide: A solution of tert-butyl (R)-(2-(((3-(3-(4,4-difluoroazepan-1-yl)-5-methyl-6-(trifluoromethyl)pyridazine-4-carboxamido)phenyl)(methyl)(oxo)-λ⁶-sulfaneylidene)amino)-2-oxoethyl)carbamate (70 mg, 0.11 mmol) in DCM (5 mL) was added TFA (0.5 mL) at room temperature. The reaction mixture was stirred at room temperature for 2 hours. After the reaction was completed, the solution was diluted with water (10 mL) and extracted with DCM (10 mL×3). The combined organic layers were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.05% NH₃) to obtain (R)-3-(4,4-difluoroazepan-1-yl)-N-(3-(N-glycyl-S-methylsulfonimidoyl)phenyl)-5-methyl-6-(trifluoromethyl)pyridazine-4-carboxamide (18.5 mg, 30.6%) as a white solid. ¹H NMR (400 MHz, DMSO-d₆) δ 11.20 (s, 1H), 8.26 (s, 1H), 7.96 (d, J=7.7 Hz, 1H), 7.79-7.61 (m, 2H), 3.85 (s, 2H), 3.69 (s, 2H), 3.46 (s, 3H), 3.23 (s, 2H), 2.38-2.27 (m, 5H), 2.12-2.01 (m, 2H), 1.93-1.85 (m, 2H). LCMS (ESI) calcd. for C₂₂H₂₆F₅N₆O₃S [M+H]⁺ m/z 549.17, found 549.10.

### Example 178

### (R)-2-(4,4-difluoroazepan-1-yl)-4-methyl-5-(2-methyloxazol-5-yl)-N-(3-(S-methylsulfonimidoyl)phenyl)nicotinamide

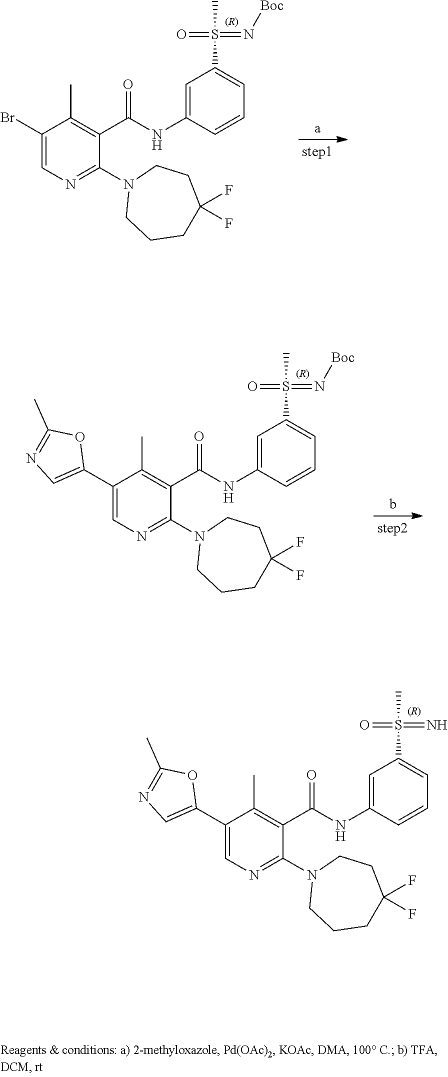

Step 1: tert-butyl (R)-((3-(2-(4,4-difluoroazepan-1-yl)-4-methyl-5-(2-methyloxazol-5-yl)nicotinamido)phenyl)(methyl)(oxo)-λ⁶-sulfaneylidene)carbamate: A mixture of tert-butyl (R)-((3-(5-bromo-2-(4,4-difluoroazepan-1-yl)-4-methylnicotinamido)phenyl)(methyl)(oxo)-λ⁶-sulfaneylidene)carbamate (100 mg, 0.167 mmol) and 2-methyloxazole (50 mg, 0.501 mmol) in DMA (10 mL) was added Pd(OAc)₂ (5 mg, 0.017 mmol) and KOAc (60 mg, 0.501 mmol). The mixture was heated at 100° C. for 6 hours under an atmosphere of N₂. The reaction was monitored by LCMS. After the reaction was completed, the mixture was cooled to room temperature. The resulting solution was diluted with water (20 mL) and extracted with DCM (20 mL×3). The combined organic phases were washed with brine, dried over Na₂SO₄, concentrated under vacuum. The residue was purified by flash column chromatography on silica gel (PE/EtOAc=2/1 to 1/1) to give tert-butyl (R)-((3-(2-(4,4-difluoroazepan-1-yl)-4-methyl-5-(2-methyloxazol-5-yl)nicotinamido)phenyl)(methyl)(oxo)-λ⁶-sulfaneylidene)carbamate (90 mg, 49.00%) as a yellow solid. LCMS (ESI) calcd. for C₂₉H₃₆F₂N₅O₅S [M+H]⁺ m/z 604.24, found 604.25.

Step 2: (R)-2-(4,4-difluoroazepan-1-yl)-4-methyl-5-(2-methyloxazol-5-yl)-N-(3-(S-methylsulfonimidoyl)phenyl)nicotinamide: A solution of tert-butyl (R)-((3-(2-(4,4-difluoroazepan-1-yl)-4-methyl-5-(2-methyloxazol-5-yl)nicotinamido)phenyl)(methyl)(oxo)-λ⁶-sulfaneylidene)carbamate (90 mg, 0.149 mmol) in DCM (5 mL) was added TFA (0.5 mL) at room temperature. The reaction mixture was stirred at room temperature for 2 hours. After the reaction was completed, the solution was diluted with water (10 mL) and extracted with DCM (10 mL×3). The combined organic layers were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.1% formic acid) to (R)-2-(4,4-difluoroazepan-1-yl)-4-methyl-5-(2-methyloxazol-5-yl)-N-(3-(S-methylsulfonimidoyl)phenyl)nicotinamide (44.7 mg, 50.10%) as a white solid. ¹H NMR (400 MHz, DMSO-d₆) δ 10.91 (s, 1H), 8.36-8.35 (m, 2H), 7.91 (d, J=8.8 Hz, 1H), 7.68 (d, J=8.0 Hz, 1H), 7.61-7.57 (m, 1H), 7.22 (s, 1H), 4.23 (s, 1H), 3.69-3.66 (m, 2H), 3.61-3.58 (m, 2H), 3.06 (s, 3H), 2.47 (s, 3H), 2.33-2.32 (m, 2H), 2.29 (s, 3H), 2.00-1.99 (m, 2H), 1.83-1.82 (m, 2H). LCMS (ESI) calcd. for C₂₄H₂₈F₂N₅O₃S [M+H]⁺ m/z 504.19, found 504.15.

### Intermediate 3

### 2-((tert-butyldimethylsilyl)oxy)acetic acid

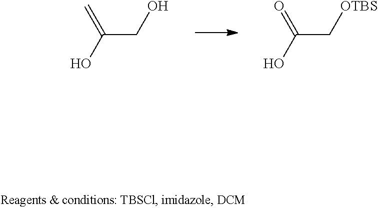

2-((tert-butyldimethylsilyl)oxy)acetic acid: To a solution of 2-hydroxyacetic acid (1 g, 13.16 mmol) and imidazole (2.68 g, 39.47 mmol) in DCM (15 mL) was added TBSCl (2.38 g, 15.79 mmol) at 0° C. Then the mixture was stirred at 25° C. for 16 hours. After the reaction was completed. The mixture was diluted with water and extracted with DCM (3×30 mL). The combined organic layers were washed with brine, dried with Na₂SO₄, and concentrated under vacuum to give crude 2-((tert-butyldimethylsilyl)oxy)acetic acid (1.6 g) which was used directly in next step without further purification.

### Intermediate 4

### (S)-2-(tert-butyldimethylsily)oxy)propanoic acid

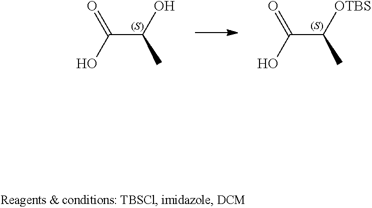

To a solution of (S)-2-hydroxypropanoic acid (1 g, 11.11 mmol) and imidazole (2.27 g, 33.32 mmol) in DCM (15 mL) was added TBSCl (2.00 g, 13.33 mmol) at 0° C. Then the mixture was heated at 25° C. for 16 hours. After the reaction was completed, the mixture was diluted with water and extracted with DCM (3×30 mL). The combined organic layers were washed with brine, dried with Na₂SO₄, and concentrated under vacuum to give crude (S)-2-((tert-butyldimethylsilyl)oxy)propanoic acid (1.4 g) which was used directly in next step without further purification.

### Example 179

### 3-(4,4-difluoroazepan-1-yl)-N-(3-((R)-N-((R)-2-hydroxypropanoyl)-S-methylsulfonimidoyl)phenyl)-5-methyl-6-(trifluoromethyl)pyridazine-4-carboxamide

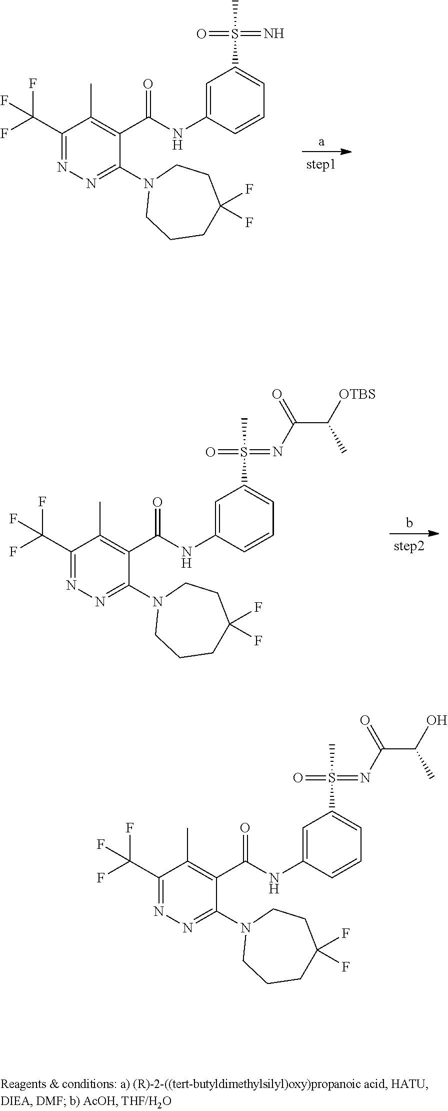 [SMILES: Cc1c(C(F)(F)F)nnc(N2CCCC(F)(F)CC2)c1C(=O)Nc1cccc([S@](C)(=N)=O)c1 ; Cc1c(C(F)(F)F)nnc(N2CCCC(F)(F)CC2)c1C(=O)Nc1cccc([S@@](C)(=O)=NC(=O)[C@@H](C)O[Si](C)(C)C(C)(C)C)c1 ; Cc1c(C(F)(F)F)nnc(N2CCCC(F)(F)CC2)c1C(=O)Nc1cccc([S@@](C)(=O)=NC(=O)[C@@H](C)O)c1]

Step 1: N-(3-((R)-N-((R)-2-((tert-butyldimethylsilyl)oxy)propanoyl)-S-methylsulfonimidoyl)phenyl)-3-(4,4-difluoroazepan-1-yl)-5-methyl-6-(trifluoromethyl)pyridazine-4-carboxamide: A mixture of (R)-3-(4,4-difluoroazepan-1-yl)-5-methyl-N-(3-(S-methylsulfonimidoyl)phenyl)-6-(trifluoromethyl)pyridazine-4-carboxamide (100 mg, 0.20 mmol) and (R)-2-((tert-butyldimethylsilyl)oxy)propanoic acid (83 mg, 0.41 mmol) in DMF (5 mL) was added HATU (116 mg, 0.31 mmol) and DIEA (79 mg, 0.61 mmol). The mixture was stirred at room temperature for 16 hours. The reaction was monitored by LCMS. After the reaction was completed, the resulting solution was diluted with water (50 mL) and extracted with DCM (20 mL×3). The combined organic phases were washed with brine, dried over Na₂SO₄, concentrated under vacuum. The residue was purified by flash column chromatography on silica gel (PE/EtOAc=2/1 to 1/1) to give N-(3-((R)-N-((R)-2-((tert-butyldimethylsilyl)oxy)propanoyl)-S-methylsulfonimidoyl)phenyl)-3-(4,4-difluoroazepan-1-yl)-5-methyl-6-(trifluoromethyl)pyridazine-4-carboxamide (80 mg, 52.2%) as a yellow solid. LCMS (ESI) calcd. for C₂₉H₄₁F₅N₅O₄SSi [M+H]⁺ m/z 678.26, found 678.20.

Step 2: 3-(4,4-difluoroazepan-1-yl)-N-(3-((R)-N-((R)-2-hydroxypropanoyl)-S-methylsulfonimidoyl)phenyl)-5-methyl-6-(trifluoromethyl)pyridazine-4-carboxamide: A solution of N-(3-((R)-N-((R)-2-((tert-butyldimethylsilyl)oxy)propanoyl)-S-methylsulfonimidoyl)phenyl)-3-(4,4-difluoroazepan-1-yl)-5-methyl-6-(trifluoromethyl)pyridazine-4-carboxamide (80 mg, 0.12 mmol) in THE (3 mL) and H₂O (1 mL) was added AcOH (3 mL) at room temperature. The reaction mixture was stirred at room temperature for 2 hours. After the reaction was completed, the solution was diluted with water (30 mL) and extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 95% MeCN/H₂O containing 0.05% NH₃) to obtain 3-(4,4-difluoroazepan-1-yl)-N-(3-((R)-N-((R)-2-hydroxypropanoyl)-S-methylsulfonimidoyl)phenyl)-5-methyl-6-(trifluoromethyl)pyridazine-4-carboxamide (29.2 mg, 43.1%) as a white solid. ¹H NMR (400 MHz, DMSO-d₆) δ 11.22 (s, 1H), 8.30 (d, J=1.8 Hz, 1H), 7.98-7.91 (m, 1H), 7.77-7.66 (m, 2H), 4.74 (d, J=5.4 Hz, 1H), 4.03 (dd, J=6.8, 5.4 Hz, 1H), 3.85 (s, 2H), 3.68 (t, J=5.9 Hz, 2H), 3.49 (s, 3H), 2.41-2.29 (m, 5H), 2.14-2.01 (m, 2H), 1.94-1.83 (m, 2H), 1.27 (d, J=6.8 Hz, 3H). LCMS (ESI) calcd. for C₂₃H₂₇F₅N₅O₄S [M+H]⁺ m/z 564.17, found 564.05.

### Example 180

### (R)-N-(3-(N-(1-aminocyclobutane-1-carbonyl)-S-methylsulfonimidoyl)phenyl)-2-(4,4-difluoroazepan-1-yl)-4-methyl-5-(trifluoromethyl)nicotinamide

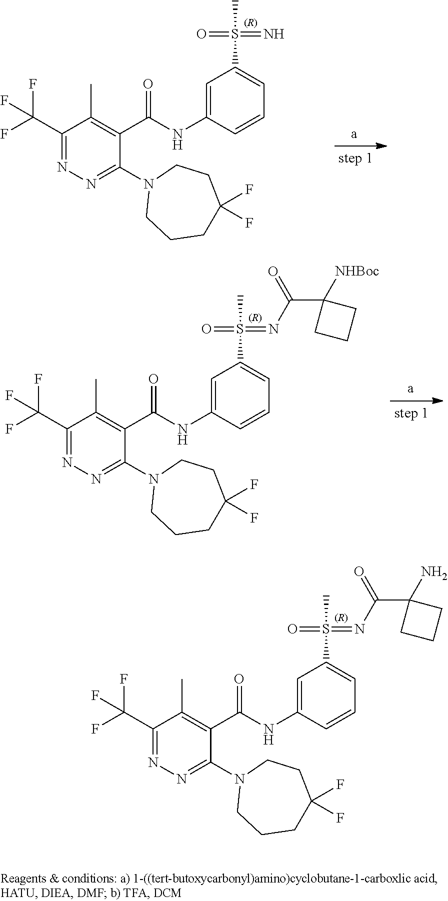

Step 1: tert-butyl (R)-(1-(((3-(2-(4,4-difluoroazepan-1-yl)-4-methyl-5-(trifluoromethyl)nicotinamido)phenyl)(methyl)(oxo)-λ⁶-sulfaneylidene)carbamoyl)cyclobutyl)carbamate: A mixture of (R)-2-(4,4-difluoroazepan-1-yl)-4-methyl-N-(3-(S-methylsulfonimidoyl)phenyl)-5-(trifluoromethyl)nicotinamide (50 mg, 0.10 mmol) and 1-((tert-butoxycarbonyl)amino)cyclobutane-1-carboxylic acid (37 mg, 0.17 mmol) in DMF (5 mL) was added HATU (50 mg, 0.13 mmol) and DIEA (42 mg, 0.32 mmol). The mixture was stirred at room temperature for 4 hours. The reaction was monitored by LCMS. After the reaction was completed, the resulting solution was diluted with water (40 mL) and extracted with DCM (20 mL×3). The combined organic phases were washed with brine, dried over Na₂SO₄, concentrated under vacuum. The residue was purified by flash column chromatography on silica gel (PE/EtOAc=2/1 to 1/1) to give tert-butyl (R)-(1-(((3-(2-(4,4-difluoroazepan-1-yl)-4-methyl-5-(trifluoromethyl)nicotinamido)phenyl)(methyl)(oxo)-λ⁶-sulfaneylidene)carbamoyl)cyclobutyl)carbamate (40 mg, 62.5%) as a white oil. LCMS (ESI) calcd. for C₃₁H₃₉F₅N₅O₅S [M+H]⁺ m/z 688.26, found 688.20.

Step 2: (R)-N-(3-(N-(1-aminocyclobutane-1-carbonyl)-S-methylsulfonimidoyl)phenyl)-2-(4,4-difluoroazepan-1-yl)-4-methyl-5-(trifluoromethyl)nicotinamide. A solution of tert-butyl (R)-(1-(((3-(2-(4,4-difluoroazepan-1-yl)-4-methyl-5-(trifluoromethyl)nicotinamido)phenyl)(methyl)(oxo)-λ⁶-sulfaneylidene)carbamoyl)cyclobutyl)carbamate (40 mg, 0.06 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.1% formic acid) to obtain (R)-N-(3-(N-(1-aminocyclobutane-1-carbonyl)-S-methylsulfonimidoyl)phenyl)-2-(4,4-difluoroazepan-1-yl)-4-methyl-5-(trifluoromethyl)nicotinamide (26.7 mg, 72.9%) as a white solid. ¹H NMR (400 MHz, DMSO-d₆) δ 11.04 (s, 1H), 8.46 (s, 1H), 8.40 (s, 1H), 7.93 (d, J=7.2 Hz, 1H), 7.72-7.66 (m, 2H), 3.74-3.71 (m, 2H), 3.62-3.60 (m, 2H), 3.48 (s, 3H), 2.34-2.30 (m, 5H), 2.03-2.00 (m, 2H), 1.98-1.94 (m, 2H), 1.79-1.70 (m, 6H). LCMS (ESI) calcd. for C₂₆H₃₁F₅N₅O₃S [M+H]⁺ m/z 588.21, found 588.10.

### Example 181

### (R)-2-(4,4-difluoroazepan-1-yl)-N-(3-(N-glycyl-S-methylsulfonimidoyl)phenyl)-4-methyl-5-(trifluoromethyl)nicotinamide

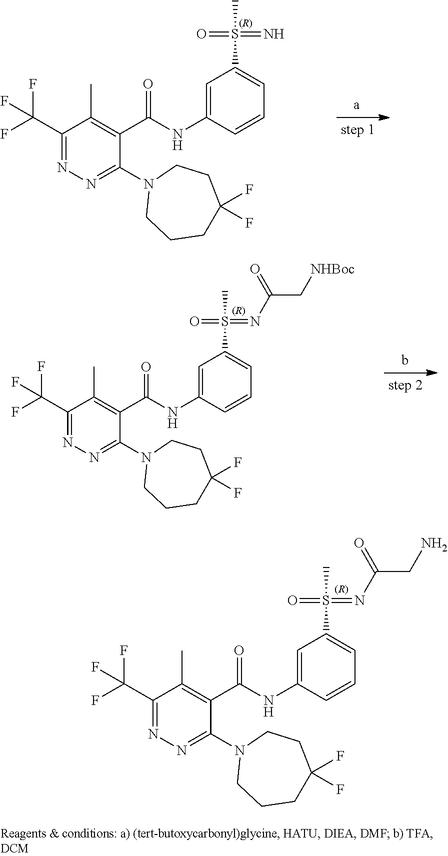

Step 1: tert-butyl (R)-(2-(((3-(2-(4,4-difluoroazepan-1-yl)-4-methyl-5-(trifluoromethyl)nicotinamido)phenyl)(methyl)(oxo)-λ⁶-sulfaneylidene)amino)-2-oxoethyl)carbamate: A mixture of (R)-2-(4,4-difluoroazepan-1-yl)-4-methyl-N-(3-(S-methylsulfonimidoyl)phenyl)-5-(trifluoromethyl)nicotinamide (50 mg, 0.10 mmol) and (tert-butoxycarbonyl)glycine (30 mg, 0.17 mmol) in DMF (5 mL) was added HATU (50 mg, 0.13 mmol) and DIEA (42 mg, 0.32 mmol). The mixture was stirred at room temperature for 4 hours. The reaction was monitored by LCMS. After the reaction was completed, the resulting solution was diluted with water (20 mL) and extracted with DCM (20 mL×3). The combined organic phases were washed with brine, dried over Na₂SO₄, concentrated under vacuum. The residue was purified by flash column chromatography on silica gel (PE/EtOAc=2/1 to 1/1) to give tert-butyl (R)-(2-(((3-(2-(4,4-difluoroazepan-1-yl)-4-methyl-5-(trifluoromethyl)nicotinamido)phenyl)(methyl)(oxo)-λ⁶-sulfaneylidene)amino)-2-oxoethyl)carbamate (40 mg, 62.5%) as a white oil. LCMS (ESI) calcd. for C₂₈H₃₅F₅N₅O₅S [M+H]⁺ m/z 648.23, found 648.10.

Step 2: (R)-2-(4,4-difluoroazepan-1-yl)-N-(3-(N-glycyl-S-methylsulfonimidoyl)phenyl)-4-methyl-5-(trifluoromethyl)nicotinamide: A solution of tert-butyl (R)-(2-(((3-(2-(4,4-difluoroazepan-1-yl)-4-methyl-5-(trifluoromethyl)nicotinamido)phenyl)(methyl)(oxo)-λ⁶-sulfaneylidene)amino)-2-oxoethyl)carbamate (40 mg, 0.06 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. The combined organic phases were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.1% formic acid) to obtain (R)-2-(4,4-difluoroazepan-1-yl)-N-(3-(N-glycyl-S-methylsulfonimidoyl)phenyl)-4-methyl-5-(trifluoromethyl)nicotinamide (17.5 mg, 54.7%) as a white solid. ¹H NMR (400 MHz, DMSO-d₆) δ 11.03 (s, 1H), 8.46 (s, 1H), 8.30 (s, 1H), 7.99 (d, J=7.2 Hz, 1H), 7.71-7.65 (m, 2H), 3.74-3.71 (m, 2H), 3.62-3.60 (m, 2H), 3.46 (s, 3H), 3.24 (s, 2H), 2.32 (s, 3H), 2.32-2.30 (m, 2H), 2.03-2.00 (m, 2H), 1.88-1.83 (m, 2H). LCMS (ESI) calcd. for C₂₃H₂₇F₅N₅O₃S [M+H]⁺ m/z 548.18, found 548.10.

### Example 182

### N-(3-((R)-N-(D-alanyl)-S-methylsulfonimidoyl)phenyl)-2-(4,4-difluoroazepan-1-yl)-4-methyl-5-(trifluoromethyl)nicotinamide

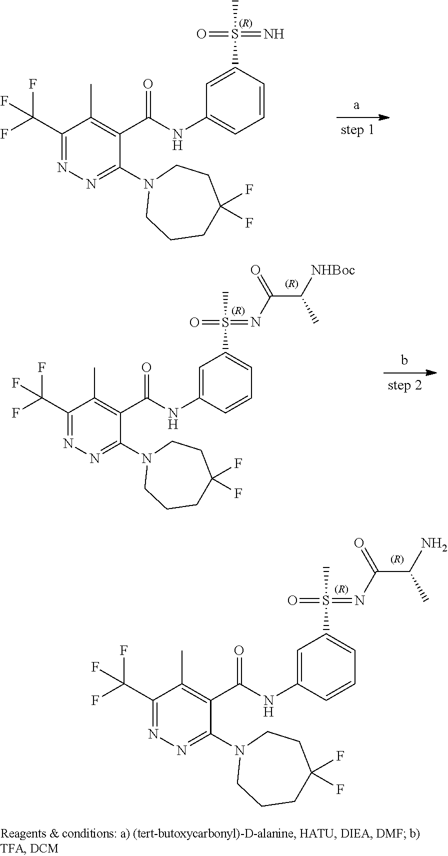

Step 1: tert-butyl ((R)-1-(((R)-(3-(2-(4,4-difluoroazepan-1-yl)-4-methyl-5-(trifluoromethyl)nicotinamido)phenyl)(methyl)(oxo)-λ⁶-sulfaneylidene)amino)-1-oxopropan-2-yl)carbamate: A mixture of (R)-2-(4,4-difluoroazepan-1-yl)-4-methyl-N-(3-(S-methylsulfonimidoyl)phenyl)-5-(trifluoromethyl)nicotinamide (60 mg, 0.12 mmol), (tert-butoxycarbonyl)-D-alanine (35 mg, 0.18 mmol) and HATU (91 mg, 0.24 mmol) in DMF (3 mL) was added DIEA (46 mg, 0.36 mmol) at room temperature. The reaction mixture was stirred at room temperature for 4 hours. After the reaction was completed, the resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtAOc=1/1) to provide tert-butyl ((R)-1-(((R)-(3-(2-(4,4-difluoroazepan-1-yl)-4-methyl-5-(trifluoromethyl)nicotinamido)phenyl)(methyl)(oxo)-λ⁶-sulfaneylidene)amino)-1-oxopropan-2-yl)carbamate (75 mg, 82.4%) as a yellow solid. LCMS (ESI) calcd. for C₂₉H₃₇F₅N₅O₅S [M+H]⁺ m/z 662.25, found 662.10.

Step 2: N-(3-((R)-N-(D-alanyl)-S-methylsulfonimidoyl)phenyl)-2-(4,4-difluoroazepan-1-yl)-4-methyl-5-(trifluoromethyl)nicotinamide: To a solution of tert-butyl ((R)-1-(((R)-(3-(2-(4,4-difluoroazepan-1-yl)-4-methyl-5-(trifluoromethyl)nicotinamido)phenyl)(methyl)(oxo)-λ⁶-sulfaneylidene)amino)-1-oxopropan-2-yl)carbamate (75 mg, 0.11 mmol) in DCM (3 mL) was added TFA (0.3 mL). The mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.05% NH₃) to provide N-(3-((R)-N-(D-alanyl)-S-methylsulfonimidoyl)phenyl)-2-(4,4-difluoroazepan-1-yl)-4-methyl-5-(trifluoromethyl)nicotinamide (20 mg, 31.45%) as a white solid. ¹H NMR (400 MHz, DMSO-d₆) δ 11.03 (s, 1H), 8.46 (s, 1H), 8.34 (s, 1H), 7.97 (d, J=7.6 Hz, 1H), 7.73-7.63 (m, 2H), 3.72 (s, 2H), 3.62 (t, J=5.8 Hz, 2H), 3.46 (s, 3H), 2.36-2.23 (m, 5H), 2.07-1.94 (m, 3H), 1.89-1.80 (m, 2H), 1.18 (d, J=6.9 Hz, 3H). LCMS (ESI) calcd. for C₂₄H₂₉F₅N₅O₃S [M+H]⁺ m/z 562.19, found 562.15.

### Example 183

### N-(3-((R)-N-(D-alanyl)-S-methylsulfonimidoyl)phenyl)-3-(4,4-difluoroazepan-1-yl)-5-methyl-6-(trifluoromethyl)pyridazine-4-carboxamide

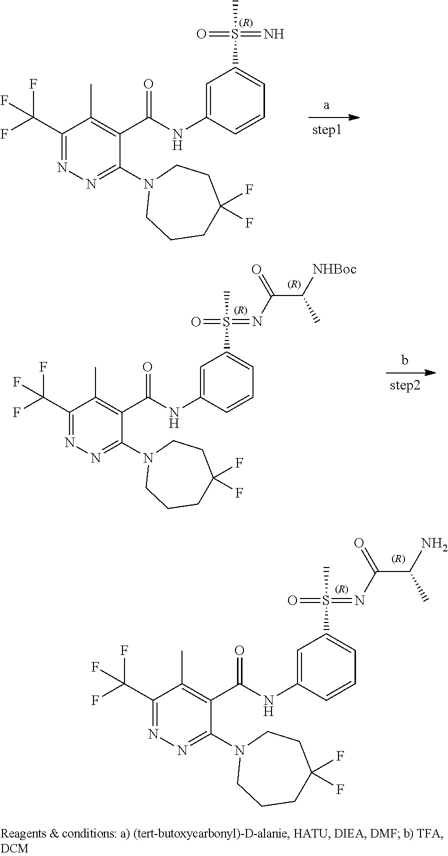

Step 1: tert-butyl ((R)-1-(((R)-(3-(3-(4,4-difluoroazepan-1-yl)-5-methyl-6-(trifluoromethyl)pyridazine-4-carboxamido)phenyl)(methyl)(oxo)-λ⁶-sulfaneylidene)amino)-1-oxopropan-2-yl)carbamate: A mixture of (R)-3-(4,4-difluoroazepan-1-yl)-5-methyl-N-(3-(S-methylsulfonimidoyl)phenyl)-6-(trifluoromethyl)pyridazine-4-carboxamide (100 mg, 0.20 mmol) and (tert-butoxycarbonyl)-D-alanine (77 mg, 0.41 mmol) in DMF (5 mL) was added HATU (116 mg, 0.31 mmol) and DIEA (79 mg, 0.61 mmol). The mixture was stirred at room temperature for 16 hours. The reaction was monitored by LCMS. After the reaction was completed, the resulting solution was diluted with water (40 mL) and extracted with DCM (20 mL×3). The combined organic phases were washed with brine, dried over Na₂SO₄, concentrated under vacuum. The residue was purified by flash column chromatography on silica gel (PE/EtOAc=2/1 to 1/1) to give tert-butyl ((R)-1-(((R)-(3-(3-(4,4-difluoroazepan-1-yl)-5-methyl-6-(trifluoromethyl)pyridazine-4-carboxamido)phenyl)(methyl)(oxo)-λ⁶-sulfaneylidene)amino)-1-oxopropan-2-yl)carbamate (80 mg, 53.4%) as a yellow solid. LCMS (ESI) calcd. for C₂₈H₃₆F₅N₆O₅S [M+H]⁺ m/z 663.24, found 663.25.

Step 2: N-(3-((R)-N-(D-alanyl)-S-methylsulfonimidoyl)phenyl)-3-(4,4-difluoroazepan-1-yl)-5-methyl-6-(trifluoromethyl)pyridazine-4-carboxamide. A solution of tert-butyl ((R)-1-(((R)-(3-(3-(4,4-difluoroazepan-1-yl)-5-methyl-6-(trifluoromethyl)pyridazine-4-carboxamido)phenyl)(methyl)(oxo)-λ⁶-sulfaneylidene)amino)-1-oxopropan-2-yl)carbamate (80 mg, 0.12 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 95% MeCN/H₂O containing 0.05% NH₃) to N-(3-((R)-N-(D-alanyl)-S-methylsulfonimidoyl)phenyl)-3-(4,4-difluoroazepan-1-yl)-5-methyl-6-(trifluoromethyl)pyridazine-4-carboxamide (26.4 mg, 38.1%) as a white solid. ¹H NMR (400 MHz, DMSO-d₆) δ11.24 (s, 1H), 8.36 (s, 1H), 7.94 (d, J=8.1 Hz, 1H), 7.82-7.68 (m, 2H), 6.72 (s, 2H), 3.85 (s, 2H), 3.73-3.64 (m, 3H), 3.56 (s, 3H), 2.40-2.30 (m, 5H), 2.13-2.01 (m, 2H), 1.93-1.83 (m, 2H), 1.35 (d, J=7.1 Hz, 3H). LCMS (ESI) calcd. for C₂₃H₂₈F₅N₆O₃S [M+H]⁺ m/z 563.19, found 563.10.

### Example 184

### (R)-2-(4,4-difluoroazepan-1-yl)-N-(3-(N-glycyl-S-methylsulfonimidoyl)phenyl)-4-methyl-5-(1-methyl-1H-pyrazol-4-yl)-6-(trifluoromethyl)nicotinamide

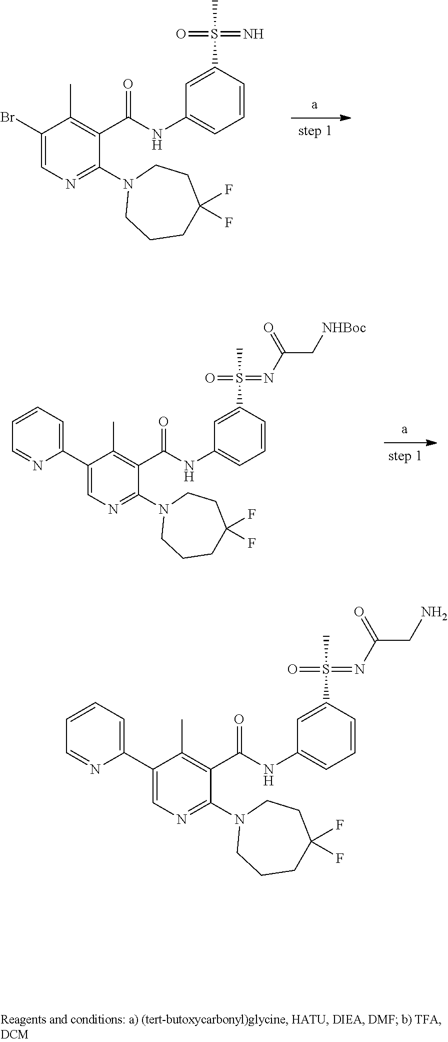 [SMILES: Cc1c(Br)cnc(N2CCCC(F)(F)CC2)c1C(=O)Nc1cccc([S@](C)(=N)=O)c1 ; Cc1c(-c2ccccn2)cnc(N2CCCC(F)(F)CC2)c1C(=O)Nc1cccc([S@@](C)(=O)=NC(=O)CNC(=O)OC(C)(C)C)c1 ; Cc1c(-c2ccccn2)cnc(N2CCCC(F)(F)CC2)c1C(=O)Nc1cccc([S@@](C)(=O)=NC(=O)CN)c1]

Step 1: tert-butyl (R)-(2-(((3-(2-(4,4-difluoroazepan-1-yl)-4-methyl-5-(1-methyl-1H-pyrazol-4-yl)-6-(trifluoromethyl)nicotinamido)phenyl)(methyl)(oxo)-λ⁶-sulfaneylidene)amino)-2-oxoethyl)carbamate: A mixture of (R)-2-(4,4-difluoroazepan-1-yl)-4-methyl-5-(1-methyl-1H-pyrazol-4-yl)-N-(3-(S-methylsulfonimidoyl)phenyl)-6-(trifluoromethyl)nicotinamide (100 mg, 0.19 mmol) and (tert-butoxycarbonyl)glycine (51 mg, 0.29 mmol) in DMF (5 mL) was added HATU (147 mg, 0.39 mmol) and DIEA (75 mg, 0.58 mmol). The mixture was stirred at room temperature for 16 hours. The reaction was monitored by LCMS. After the reaction was completed, the resulting solution was diluted with water (20 mL) and extracted with DCM (20 mL×3). The combined organic phases were washed with brine, dried over Na₂SO₄, concentrated under vacuum. The residue was purified by flash column chromatography on silica gel (PE/EtOAc=2/1 to 1/1) to give tert-butyl (R)-(2-(((3-(2-(4,4-difluoroazepan-1-yl)-4-methyl-5-(1-methyl-1H-pyrazol-4-yl)-6-(trifluoromethyl)nicotinamido)phenyl)(methyl)(oxo)-λ⁶-sulfaneylidene)amino)-2-oxoethyl)carbamate (80 mg, 57.0%) as a yellow solid. LCMS (ESI) calcd. for C₃₂H₃₉F₅N₇O₅S [M+H]⁺ m/z 728.27, found 728.20

Step 2: (R)-2-(4,4-difluoroazepan-1-yl)-N-(3-(N-glycyl-S-methylsulfonimidoyl)phenyl)-4-methyl-5-(1-methyl-1H-pyrazol-4-yl)-6-(trifluoromethyl)nicotinamide: A solution of tert-butyl (R)-(2-(((3-(2-(4,4-difluoroazepan-1-yl)-4-methyl-5-(1-methyl-1H-pyrazol-4-yl)-6-(trifluoromethyl)nicotinamido)phenyl)(methyl)(oxo)-λ⁶-sulfaneylidene)amino)-2-oxoethyl)carbamate (80 mg, 0.11 mmol) in DCM (5 mL) was added TFA (0.5 mL) at room temperature. The reaction mixture was stirred at room temperature for 2 hours. After the reaction was completed, the solution was diluted with water (10 mL) and extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 80% MeCN/H₂O containing 0.1% NH₃·H₂O) to obtain (R)-2-(4,4-difluoroazepan-1-yl)-N-(3-(N-glycyl-S-methylsulfonimidoyl)phenyl)-4-methyl-5-(1-methyl-1H-pyrazol-4-yl)-6-(trifluoromethyl)nicotinamide (30.5 mg, 44.2%) as a white solid. 1H NMR (400 MHz, DMSO-d₆) δ 11.06 (s, 1H), 8.39-8.28 (m, 1H), 8.04-7.91 (m, 1H), 7.75-7.62 (m, 3H), 7.36 (s, 1H), 3.90 (s, 3H), 3.71-3.56 (m, 4H), 3.45 (s, 3H), 3.23 (s, 2H), 2.38-2.24 (m, 2H), 2.09-1.96 (m, 5H), 1.88-1.79 (m, 2H). LCMS (ESI) calcd. for C₂₇H₃₀F₅N₇O₃S [M+H]⁺ m/z 628.22, found 628.15.

### Example 185 and Example 186

### (S)-2-(4,4-difluoroazepan-1-yl)-4,6-dimethyl-N-(3-(S-methylsulfonimidoyl)phenyl)-5-(trifluoromethyl)nicotinamide and (R)-2-(4,4-difluoroazepan-1-yl)-4,6-dimethyl-N-(3-(S-methylsulfonimidoyl)phenyl)-5-(trifluoromethyl)nicotinamide

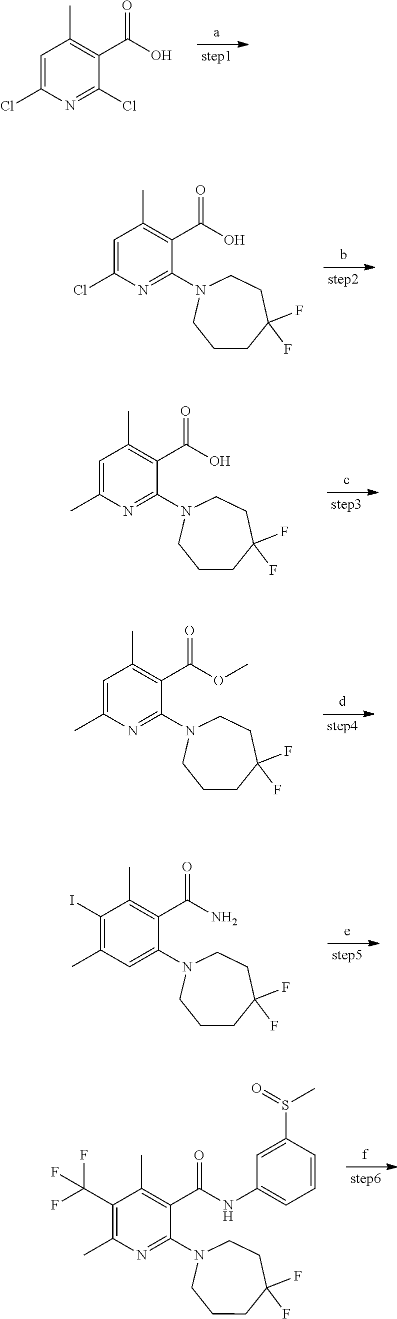 [SMILES: Cc1cc(Cl)nc(Cl)c1C(=O)O ; Cc1cc(Cl)nc(N2CCCC(F)(F)CC2)c1C(=O)O ; Cc1cc(C)c(C(=O)O)c(N2CCCC(F)(F)CC2)n1 ; COC(=O)c1c(C)cc(C)nc1N1CCCC(F)(F)CC1 ; Cc1cc(N2CCCC(F)(F)CC2)c(C(N)=O)c(C)c1I ; Cc1nc(N2CCCC(F)(F)CC2)c(C(=O)Nc2cccc(S(C)=O)c2)c(C)c1C(F)(F)F]

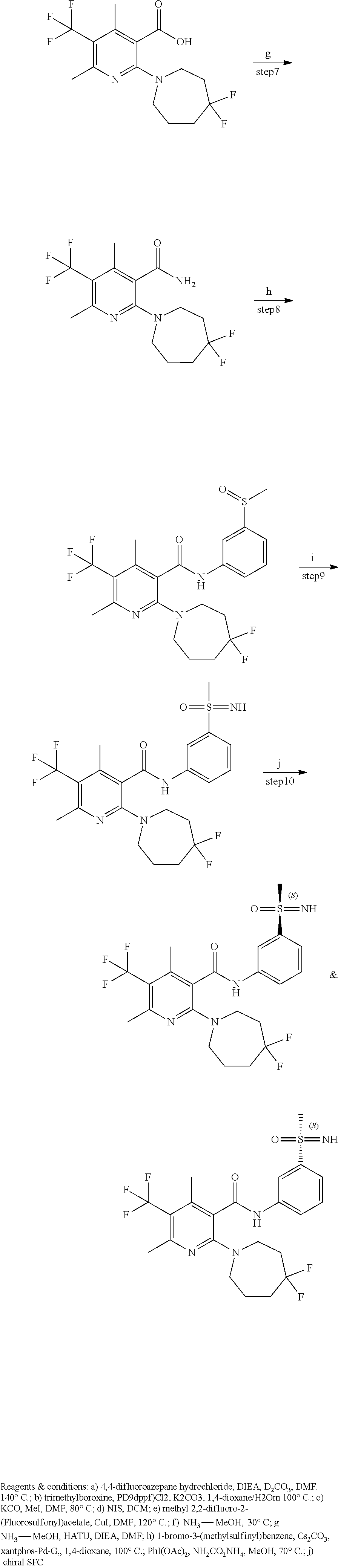

Step 1: 6-chloro-2-(4,4-difluoroazepan-1-yl)-4-methylnicotinic acid: A mixture of 2,6-dichloro-4-methylnicotinic acid (4.0 g, 19.51 mmol), 4,4-difluoroazepane hydrochloride (4.0 g, 23.41 mmol), K₂CO₃ (8.0 g, 58.53 mmol) and DIEA (6.3 g, 48.78 mmol) in NMP (60 mL) was heated at 140° C. for 8 hours under an atmosphere of N₂. The reaction was monitored by LCMS. After the reaction was completed, the mixture was cooled to room temperature. The resulting solution was diluted with water (150 mL) and extracted with EtOAc (80 mL×3). The combined organic layers were washed with brine, dried over Na₂SO₄, and concentrated under vacuum. The residue was purified by flash column chromatography on silica gel (DCM/MeOH=50/1) to give 6-chloro-2-(4,4-difluoroazepan-1-yl)-4-methylnicotinic acid (4.5 g, 75.89%) as a yellow solid. LCMS (ESI) calcd. for C₁₃H₁₆ClF₂N₂O₂ [M+H]⁺ m/z 305.09, found 304.90.

Step 2: 2-(4,4-difluoroazepan-1-yl)-4,6-dimethylnicotinic acid: A mixture of 6-chloro-2-(4,4-difluoroazepan-1-yl)-4-methylnicotinic acid (4.5 g, 14.80 mmol), Trimethylboroxine (5.6 g, 44.37 mmol), K₂CO₃ (6.1 g, 44.41 mmol) and Pd(dppf)Cl₂ (1.1 g, 1.48 mmol) in 1,4-dioxane/H₂O (4/1, 60 mL) was heated at 100° C. for 16 hours under an atmosphere of N₂. The reaction was monitored by LCMS. After the reaction was completed, the mixture was cooled to room temperature. The resulting solution was diluted with water (150 mL) and extracted with EtOAc (80 mL×3). The combined organic layers were washed with brine, dried over Na₂SO₄, and concentrated under vacuum. The residue was purified by flash column chromatography on silica gel (DCM/MeOH=10/1) to give 2-(4,4-difluoroazepan-1-yl)-4,6-dimethylnicotinic acid (3.2 g, 76.19%) as a yellow solid. LCMS (ESI) calcd. for C₁₄H₁₉F₂N₂O₂ [M+H]⁺ m/z 285.14, found 284.95.

Step 3: methyl 2-(4,4-difluoroazepan-1-yl)-4,6-dimethylnicotinate: A mixture of 2-(4,4-difluoroazepan-1-yl)-4,6-dimethylnicotinic acid (3.2 g, 11.27 mmol), K₂CO₃ (4.7 g, 33.80 mmol) and Mel (3.2 g, 22.54 mmol) in DMF (40 mL) was heated at 80° C. for 4 hours. The reaction was monitored by LCMS. After the reaction was completed, the mixture was cooled to room temperature. The resulting solution was diluted with water (100 mL) and extracted with EtOAc (50 mL×3). The combined organic layers were washed with brine, dried over Na₂SO₄, and concentrated under vacuum. The residue was purified by flash column chromatography on silica gel (PE/EtOAc=1/10) to give methyl 2-(4,4-difluoroazepan-1-yl)-4,6-dimethylnicotinate (2.8 g, 83.58%) as a yellow solid. LCMS (ESI) calcd. for C₁₅H₂₁F₂N₂O₂ [M+H]⁺ m/z 299.16, found 298.75.

Step 4: methyl 2-(4,4-difluoroazepan-1-yl)-5-iodo-4,6-dimethylnicotinate: To a solution of methyl 2-(4,4-difluoroazepan-1-yl)-4,6-dimethylnicotinate (2.5 g, 8.39 mmol) in DCM (30 mL) was added NIS (2.1 g, 9.23 mmol). The mixture was stirred at room temperature for 2 hours. LCMS showed the reaction was completed. The resulting solution was concentrated under vacuum. The residue was purified by flash column chromatography on silica gel (PE/EtOAc=5/1) to give methyl 2-(4,4-difluoroazepan-1-yl)-5-iodo-4,6-dimethylnicotinate (3.1 g, 87.32%) as a yellow solid. LCMS (ESI) calcd. for C₁₅H₂₀F₂IN₂O₂ [M+H]⁺ m/z 425.05, found 424.75.

Step 5: methyl 2-(4,4-difluoroazepan-1-yl)-4,6-dimethyl-5-(trifluoromethyl)nicotinate: A mixture of methyl 2-(4,4-difluoroazepan-1-yl)-5-iodo-4,6-dimethylnicotinate (2 g, 4.72 mmol), methyl 2,2-difluoro-2-(fluorosulfonyl)acetate (4.5 g, 23.58 mmol) and CuI (1.8 g, 9.44 mmol) in DMF (30 mL) was heated at 120° C. for 3 hours. The reaction was monitored by LCMS. After the reaction was completed, the mixture was cooled to room temperature. The resulting solution was diluted with water (80 mL) and extracted with EtOAc (40 mL×3). The combined organic layers were washed with brine, dried over Na₂SO₄, and concentrated under vacuum. The residue was purified by flash column chromatography on silica gel (PE/EtOAc=5/1) to give methyl 2-(4,4-difluoroazepan-1-yl)-4,6-dimethyl-5-(trifluoromethyl)nicotinate (1.4 g, 80.46%) as a yellow solid. LCMS (ESI) calcd. for C₁₆H₂₀F₅N₂O₂ [M+H]⁺ m/z 367.14, found 366.95.

Step 6: 2-(4,4-difluoroazepan-1-yl)-4,6-dimethyl-5-(trifluoromethyl)nicotinic acid: A solution of methyl 2-(4,4-difluoroazepan-1-yl)-4,6-dimethyl-5-(trifluoromethyl)nicotinate (1.4 g, 3.81 mmol) in NH₃-MeOH (30 mL) was heated at 130° C. for 8 hours in a high-pressure reactor. The reaction was monitored by LCMS. After the reaction was completed, the mixture was cooled to room temperature. The resulting solution was concentrated under vacuum. The residue was purified by flash column chromatography on silica gel (PE/EtOAc=1/2) to give 2-(4,4-difluoroazepan-1-yl)-4,6-dimethyl-5-(trifluoromethyl)nicotinic acid (600 mg, 44.78%) as a yellow solid. LCMS (ESI) calcd. for C₁₅H₁₈F₅N₂O₂ [M+H]⁺ m/z 353.13, found 352.80.

Step 7: 2-(4,4-difluoroazepan-1-yl)-4,6-dimethyl-5-(trifluoromethyl)nicotinamide: To a mixture of 2-(4,4-difluoroazepan-1-yl)-4,6-dimethyl-5-(trifluoromethyl)nicotinic acid (600 mg, 1.70 mmol), HATU (1.3 g, 3.41 mmol) and DIEA (660 mg, 5.11 mmol) in DMF (20 mL) was added NH₃-MeOH (7M, 10 mL). The reaction was stirred at room temperature for 1 hour. The reaction was monitored by LCMS. After the reaction was completed, the mixture was cooled to room temperature. The resulting solution was diluted with water (50 mL) and extracted with EtOAc (30 mL×3). The combined organic layers were washed with brine, dried over Na₂SO₄, and concentrated under vacuum. The residue was purified by flash column chromatography on silica gel (PE/EtOAc=1/3) to give 2-(4,4-difluoroazepan-1-yl)-4,6-dimethyl-5-(trifluoromethyl)nicotinamide (440 mg, 73.58%) as a yellow solid. LCMS (ESI) calcd. for C₁₅H₁₉F₅N₃O [M+H]⁺ m/z 352.15, found 351.90.

Step 8: 2-(4,4-difluoroazepan-1-yl)-4,6-dimethyl-N-(3-(methylsulfinyl)phenyl)-5-(trifluoromethyl)nicotinamide: A mixture of 2-(4,4-difluoroazepan-1-yl)-4,6-dimethyl-5-(trifluoromethyl)nicotinamide (440 mg, 1.25 mmol), 1-bromo-3-(methylsulfinyl)benzene (410 mg, 1.88 mmol), Cs₂CO₃ (1.2 g, 3.75 mmol) and Xantphos-Pd-G2 (111 mg, 0.13 mmol) in 1,4-dioxane (10 mL) was heated at 100° C. for 16 hours under an atmosphere of N₂. The reaction was monitored by LCMS. After the reaction was completed, the mixture was cooled to room temperature. The resulting solution was concentrated under vacuum. The residue was purified by flash column chromatography on silica gel (PE/EtOAc=1/1) to give 2-(4,4-difluoroazepan-1-yl)-4,6-dimethyl-N-(3-(methylsulfinyl)phenyl)-5-(trifluoromethyl)nicotinamide (350 mg, 57.19%) as a yellow solid. LCMS (ESI) calcd. for C₂₂H₂₅F₅N₃O₂S [M+H]⁺ m/z 490.16, found 490.05.

Step 9: 2-(4,4-difluoroazepan-1-yl)-4,6-dimethyl-N-(3-(S-methylsulfonimidoyl)phenyl)-5-(trifluoromethyl)nicotinamide: A mixture of 2-(4,4-difluoroazepan-1-yl)-4,6-dimethyl-N-(3-(methylsulfinyl)phenyl)-5-(trifluoromethyl)nicotinamide (350 mg, 0.71 mmol), PhI(OAc)₂ (710 mg, 2.14 mmol) and NH₂CO₂NH₄ (167 mg, 2.14 mmol) in MeOH (10 mL) was heated at 70° C. for 16 hours under an atmosphere of N₂. The reaction was monitored by LCMS. After the reaction was completed, the mixture was cooled to room temperature. The resulting solution was concentrated under vacuum. The residue was first purified by flash column chromatography on silica gel (PE/EtOAc=1/1), then further purified by prep-HPLC (Gemini 5 um C₁₈ column, 150*21.2 mm, eluting with 50% to 95% MeCN/H₂O containing 0.05% NH₃) to give 2-(4,4-difluoroazepan-1-yl)-4,6-dimethyl-N-(3-(S-methylsulfonimidoyl)phenyl)-5-(trifluoromethyl)nicotinamide (160 mg, 44.44%) as a white solid. LCMS (ESI) calcd. for C₂₂H₂₆F₅N₄O₂S [M+H]⁺ m/z 505.17, found 505.05.

Step 10: (S)-2-(4,4-difluoroazepan-1-yl)-4,6-dimethyl-N-(3-(S-methylsulfonimidoyl)phenyl)-5-(trifluoromethyl)nicotinamide and (R)-2-(4,4-difluoroazepan-1-yl)-4,6-dimethyl-N-(3-(S-methylsulfonimidoyl)phenyl)-5-(trifluoromethyl)nicotinamide: 2-(4,4-difluoroazepan-1-yl)-4,6-dimethyl-N-(3-(S-methylsulfonimidoyl)phenyl)-5-(trifluoromethyl)nicotinamide was further purified by Chiral-Prep-HPLC (Daicel 1H, 4.6*250 mm, eluting with 20% CO₀₂-MeOH containing 0.1% NH₃) to afford first eluting isomer (S)-2-(4,4-difluoroazepan-1-yl)-4,6-dimethyl-N-(3-(S-methylsulfonimidoyl)phenyl)-5-(trifluoromethyl)nicotinamide. ¹H NMR (400 MHz, DMSO-d₆) δ 10.87 (s, 1H), 8.32 (s, 1H), 7.89 (d, J=8.0 Hz, 1H), 7.68 (d, J=7.7 Hz, 1H), 7.59 (t, J=7.9 Hz, 1H), 4.24 (s, 1H), 3.71 (s, 2H), 3.59 (d, J=5.3 Hz, 2H), 3.06 (s, 3H), 2.52 (s, 3H), 2.36-2.21 (m, 5H), 2.06-1.91 (m, 2H), 1.86-1.72 (m, 2H). LCMS (ESI) calcd. for C₂₂H₂₆F₅N₄O₂S [M+H]⁺ m/z 505.17, found 505.00 and the second eluting isomer (R)-2-(4,4-difluoroazepan-1-yl)-4,6-dimethyl-N-(3-(S-methylsulfonimidoyl)phenyl)-5-(trifluoromethyl)nicotinamide as a white solid. ¹H NMR (400 MHz, DMSO-d₆) δ 10.86 (s, 1H), 8.32 (s, 1H), 7.89 (d, J=8.1 Hz, 1H), 7.68 (d, J=7.9 Hz, 1H), 7.59 (t, J=7.9 Hz, 1H), 4.23 (s, 1H), 3.71 (s, 2H), 3.60 (t, J=5.8 Hz, 2H), 3.06 (s, 3H), 2.52 (s, 3H), 2.35-2.22 (m, 5H), 2.06-1.92 (m, 2H), 1.87-1.75 (m, 2H). LCMS (ESI) calcd. for C₂₂H₂₆F₅N₄O₂S [M+H]⁺ m/z 505.17, found 505.00.

### Example 187

### (R)-N-(3-(N-(2-amino-2-methylpropanoyl)-S-methylsulfonimidoyl)phenyl)-2-(4,4-diflu oroazepan-1-yl)-4-methyl-5-(trifluoromethyl)nicotinamide

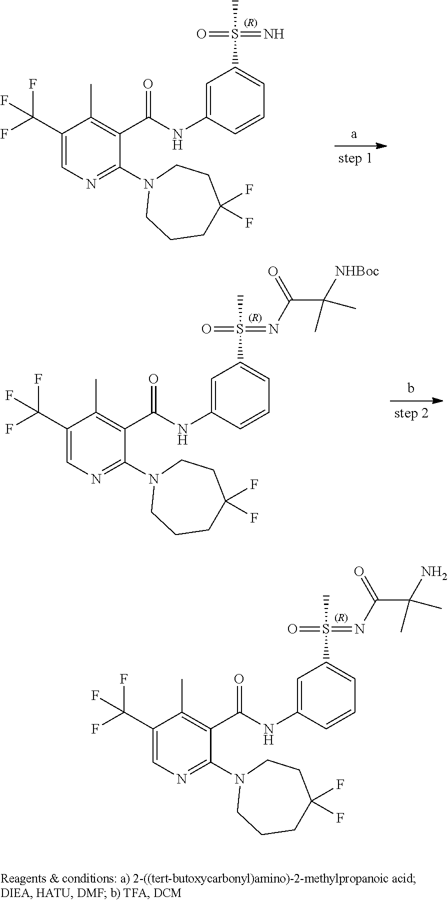

Step 1: tert-butyl (R)-(1-(((3-(2-(4,4-difluoroazepan-1-yl)-4-methyl-5-(trifluoromethyl)nicotinamido)phenyl)(methyl)(oxo)-λ⁶-sulfaneylidene)amino)-2-methyl-1-oxopropan-2-yl)carbamate: A mixture of (R)-2-(4,4-difluoroazepan-1-yl)-4-methyl-N-(3-(S-methylsulfonimidoyl)phenyl)-5-(trifluoromethyl)nicotinamide (50 mg, 0.10 mmol) and 2-((tert-butoxycarbonyl)amino)-2-methylpropanoic acid (35 mg, 0.17 mmol) in DMF (5 mL) was added HATU (50 mg, 0.13 mmol) and DIEA (42 mg, 0.32 mmol). The mixture was stirred at room temperature for 4 hours. The reaction was monitored by LCMS. After the reaction was completed, the resulting solution was diluted with water (40 mL) and extracted with DCM (20 mL×3). The combined organic layers were washed with brine, dried over Na₂SO₄, concentrated under vacuum. The residue was purified by flash column chromatography on silica gel (PE/EtOAc=2/1 to 1/1) to give tert-butyl (R)-(1-(((3-(2-(4,4-difluoroazepan-1-yl)-4-methyl-5-(trifluoromethyl)nicotinamido)phenyl)(methyl)(oxo)-λ⁶-sulfaneylidene)amino)-2-methyl-1-oxopropan-2-yl)carbamate (40 mg, 57.97%) as a white oil. LCMS (ESI) calcd. for C₃₀H₃₉F₅N₅O₅S [M+H]⁺ m/z 676.26, found 676.15.

Step 2: (R)-N-(3-(N-(2-amino-2-methylpropanoyl)-S-methylsulfonimidoyl)phenyl)-2-(4,4-difluoroazepan-1-yl)-4-methyl-5-(trifluoromethyl)nicotinamide A solution of tert-butyl (R)-(1-(((3-(2-(4,4-difluoroazepan-1-yl)-4-methyl-5-(trifluoromethyl)nicotinamido)phenyl)(methyl)(oxo)-λ⁶-sulfaneylidene)amino)-2-methyl-1-oxopropan-2-yl)carbamate (40 mg, 0.06 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.1% formic acid) to obtain (R)-N-(3-(N-(2-amino-2-methylpropanoyl)-S-methylsulfonimidoyl)phenyl)-2-(4,4-difluoroazepan-1-yl)-4-methyl-5-(trifluoromethyl)nicotinamide (15.2 mg, 44.18%) as a white solid. ¹H NMR (400 MHz, DMSO-d₆) δ 11.02 (s, 1H), 8.46 (s, 1H), 8.37 (s, 1H), 7.93 (d, J=7.3 Hz, 1H), 7.74-7.62 (m, 2H), 3.75-3.68 (m, 2H), 3.63 (t, J=6.0 Hz, 2H), 3.44 (s, 3H), 2.35-2.22 (m, 5H), 2.06-1.93 (m, 2H), 1.92-1.71 (m, 4H), 1.21 (d, J=3.9 Hz, 6H). LCMS (ESI) calcd. for C₂₅H₃₁F₅N₅O₃S [M+H]⁺ m/z 576.21, found 576.10.

### Example 188 and Example 189

### (S)-2-(4,4-difluoroazepan-1-yl)-4-methyl-N-(2-(S-methylsulfonimidoyl)pyridin-4-yl)-5-(trifluoromethyl)nicotinamide and (R)-2-(4,4-difluoroazepan-1-yl)-4-methyl-N-(2-(S-methylsulfonimidoyl)pyridin-4-yl)-5-(trifluoromethyl)nicotinamide

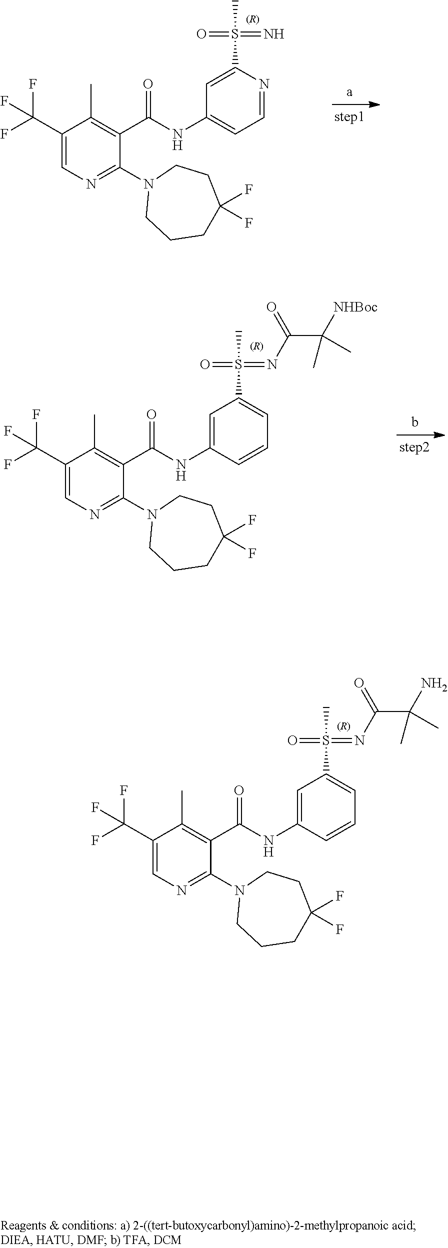

Step 1: 2-(4,4-difluoroazepan-1-yl)-4-methyl-N-(2-(methylsulfinyl)pyridin-4-yl)-5-(trifluoromethyl)nicotinamide: A solution of 2-(4,4-difluoroazepan-1-yl)-4-methyl-5-(trifluoromethyl)nicotinamide (680 mg, 2.02 mmol) in dioxane (8 mL) was added 4-bromo-2-(methylsulfinyl)pyridine (423 mg, 2.02 mmol), cesium carbonate (1970 mg, 6.06 mmol) and Xantphos-Pd-G2 (180 mg, 0.202 mmol) at room temperature. The reaction mixture was stirred at 100° C. for 16 hours under nitrogen. The reaction was monitored by LCMS. After the reaction was completed, the mixture was diluted with water (20 mL) and extracted with DCM (20 mL×3). The combine organic layers were washed with brine, dried over sodium sulfate, concentrated under vacuum. The residue was purified by flash column chromatography on silica gel (PE/EtOAc=1/1) to give 2-(4,4-difluoroazepan-1-yl)-4-methyl-N-(2-(methylsulfinyl)pyridin-4-yl)-5-(trifluoromethyl)nicotinamide (750 mg, 77.88%) as a white solid. LCMS (ESI) calcd. for C₂₀H₂₂F₅N₄O₂S [M+H]⁺ m/z 477.14, found 477.00.

Step 2: 2-(4,4-difluoroazepan-1-yl)-4-methyl-N-(2-(S-methylsulfonimidoyl)pyridin-4-yl)-5-(trifluoromethyl)nicotinamide: To a solution of 2-(4,4-difluoroazepan-1-yl)-4-methyl-N-(2-(methylsulfinyl)pyridin-4-yl)-5-(trifluoromethyl)nicotinamide (750 mg, 1.57 mmol) in MeOH (10 mL) was added PhI(OAc)₂ (1266 mg, 3.93 mmol) and ammonium carbamate (364 mg, 4.72 mmol) at room temperature. The reaction mixture was heated at 70° C. for 1 hour. After the reaction was completed, the mixture was diluted with water (25 mL), and extracted with DCM (25 mL×3). The combine organic layers were washed with brine, dried over sodium sulfate, concentrated under vacuum. The residue was first purified by flash column chromatography on silica gel (PE/EtOAc=1/1), then further purified by prep-HPLC (Gemini 5 um C₁₈ column, 150*21.2 mm, eluting with 30% to 90% MeCN/H₂O containing 0.1% formic acid) to obtain 2-(4,4-difluoroazepan-1-yl)-4-methyl-N-(2-(S-methylsulfonimidoyl)pyridin-4-yl)-5-(trifluoromethyl)nicotinamide (220 mg, 37.78%) as a white solid. ¹H NMR (400 MHz, DMSO-d₆) δ 11.34 (s, 1H), 8.63 (d, J=5.6 Hz, 1H), 8.48 (s, 1H), 8.39 (d, J=1.6 Hz, 1H), 7.82 (dd, J=5.6, 1.6 Hz, 1H), 4.40 (s, 1H), 3.71-3.69 (m, 2H), 3.57-3.55 (m, 2H), 3.15 (s, 3H), 2.32-2.30 (m, 5H), 2.02-1.95 (m, 2H), 1.84-1.81 (m, 2H). LCMS (ESI) calcd. for C₂₀H₂₃F₅N₅O₂S [M+H]⁺ m/z 492.15, found 492.05.

Step 3: (S)-2-(4,4-difluoroazepan-1-yl)-4-methyl-N-(2-(S-methylsulfonimidoyl)pyridin-4-yl)-5-(trifluoromethyl)nicotinamide and (R)-2-(4,4-difluoroazepan-1-yl)-4-methyl-N-(2-(S-methylsulfonimidoyl)pyridin-4-yl)-5-(trifluoromethyl)nicotinamide: 2-(4,4-difluoroazepan-1-yl)-4-methyl-N-(2-(S-methylsulfonimidoyl)pyridin-4-yl)-5-(trifluoromethyl)nicotinamide was further purified by Chiral-Prep-HPLC (1H-40% D-2.5, 4.6*250 mm, eluting with 35% CO₀₂-MeOH containing 0.1% NH₃) to afford first eluting isomer (S)-2-(4,4-difluoroazepan-1-yl)-4-methyl-N-(2-(S-methylsulfonimidoyl)pyridin-4-yl)-5-(trifluoromethyl)nicotinamide (53.40 mg) as a white solid. ¹H NMR (400 MHz, DMSO-d₆) δ 11.34 (s, 1H), 8.63 (d, J=5.6 Hz, 1H), 8.48 (s, 1H), 8.39 (d, J=1.6 Hz, 1H), 7.82 (dd, J=5.6, 1.6 Hz, 1H), 4.40 (s, 1H), 3.71-3.69 (m, 2H), 3.57-3.55 (m, 2H), 3.15 (s, 3H), 2.32-2.30 (m, 5H), 2.02-1.95 (m, 2H), 1.84-1.81 (m, 2H). LCMS (ESI) calcd. for C₂₀H₂₃F₅N₅O₂S [M+H]⁺ m/z 492.15, found 492.05 and the second eluting isomer (R)-2-(4,4-difluoroazepan-1-yl)-4-methyl-N-(2-(S-methylsulfonimidoyl)pyridin-4-yl)-5-(trifluoromethyl)nicotinamide (51.20 mg) as a white solid. ¹H NMR (400 MHz, DMSO-d₆) δ 11.34 (s, 1H), 8.63 (d, J=5.6 Hz, 1H), 8.48 (s, 1H), 8.39 (d, J=1.6 Hz, 1H), 7.82 (dd, J=5.6, 1.6 Hz, 1H), 4.40 (s, 1H), 3.71-3.69 (m, 2H), 3.57-3.55 (m, 2H), 3.15 (s, 3H), 2.32-2.30 (m, 5H), 2.02-1.95 (m, 2H), 1.84-1.81 (m, 2H). LCMS (ESI) calcd. for C₂₀H₂₃F₅N₅O₂S [M+H]⁺ m/z 492.15, found 492.05.

### Example 190

### N-(3-((R)-N-(L-prolyl)-S-methylsulfonimidoyl)phenyl)-2-(4,4-difluoroazepan-1-yl)-4-methyl-5-(1-methyl-1H-pyrazol-4-yl)-6-(trifluoromethyl)nicotinamide

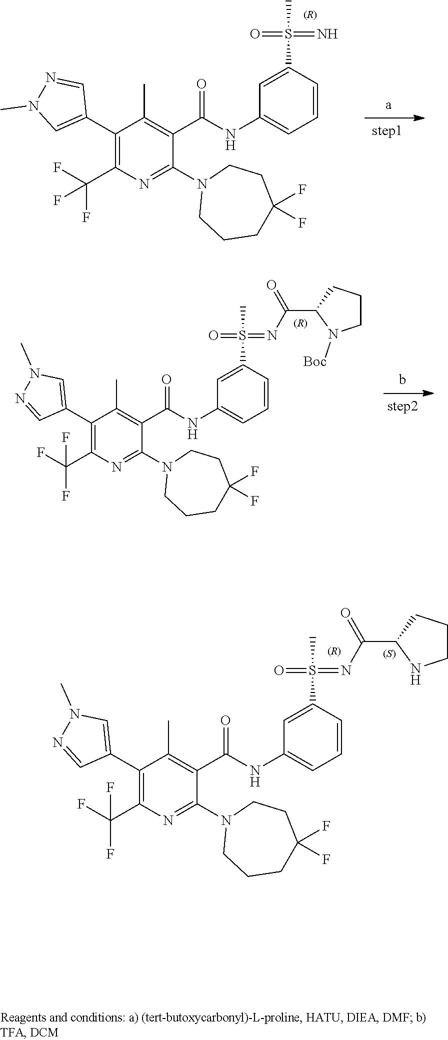

Step 1: tert-butyl (S)-2-(((R)-(3-(2-(4,4-difluoroazepan-1-yl)-4-methyl-5-(1-methyl-1H-pyrazol-4-yl)-6-(trifluoromethyl)nicotinamido)phenyl)(methyl)(oxo)-λ⁶-sulfaneylidene)carbamoyl)pyrrolidine-1-carboxylate: A mixture of (R)-2-(4,4-difluoroazepan-1-yl)-4-methyl-5-(1-methyl-1H-pyrazol-4-yl)-N-(3-(S-methylsulfonimidoyl)phenyl)-6-(trifluoromethyl)nicotinamide (100 mg, 0.19 mmol) and (tert-butoxycarbonyl)-L-proline (61 mg, 0.29 mmol) in DMF (5 mL) was added HATU (147 mg, 0.39 mmol) and DIEA (75 mg, 0.58 mmol). The mixture was stirred at room temperature for 16 hours. The reaction was monitored by LCMS. After the reaction was completed, the resulting solution was diluted with water (20 mL) and extracted with DCM (20 mL×3). The combined organic layers were washed with brine, dried over Na₂SO₄, concentrated under vacuum. The residue was purified by flash column chromatography on silica gel (PE/EtOAc=2/1 to 1/1) to give tert-butyl (S)-2-(((R)-(3-(2-(4,4-difluoroazepan-1-yl)-4-methyl-5-(1-methyl-1H-pyrazol-4-yl)-6-(trifluoromethyl)nicotinamido)phenyl)(methyl)(oxo)-λ⁶-sulfaneylidene)carbamoyl)pyrrolidine-1-carboxylate (70 mg, 48.0%) as a yellow solid. LCMS (ESI) calcd. for C₃₅H₄₃F₅N₇O₅S [M+H]⁺ m/z 768.30, found 768.20.

Step 2: N-(3-((R)-N-(L-prolyl)-S-methylsulfonimidoyl)phenyl)-2-(4,4-difluoroazepan-1-yl)-4-methyl-5-(1-methyl-1H-pyrazol-4-yl)-6-(trifluoromethyl)nicotinamide: A solution of tert-butyl (S)-2-(((R)-(3-(2-(4,4-difluoroazepan-1-yl)-4-methyl-5-(1-methyl-1H-pyrazol-4-yl)-6-(trifluoromethyl)nicotinamido)phenyl)(methyl)(oxo)-λ⁶-sulfaneylidene)carbamoyl)pyrrolidine-1-carboxylate (70 mg, 0.09 mmol) in DCM (5 mL) was added TFA (0.5 mL) at room temperature. The reaction mixture was stirred at room temperature for 2 hours. After the reaction was completed, the solution was diluted with water (10 mL) and extracted with DCM (10 mL×3). The combined organic layers were washed with brine, dried over sodium sulfate, and concentrated under vacuum. The residue was purified by prep-HPLC (Gemini 5 um C₁₈ column, 150*21.2 mm, eluting with 40% to 80% MeCN/H₂O containing 0.1% NH₃·H₂O) to obtain N-(3-((R)-N-(L-prolyl)-S-methylsulfonimidoyl)phenyl)-2-(4,4-difluoroazepan-1-yl)-4-methyl-5-(1-methyl-1H-pyrazol-4-yl)-6-(trifluoromethyl)nicotinamide (26.2 mg, 42.0%) as a white solid. ¹H NMR (400 MHz, DMSO-d₆) δ 11.08 (s, 1H), 8.40 (s, 1H), 7.92-7.85 (m, 1H), 7.73-7.63 (m, 3H), 7.36 (s, 1H), 3.90 (s, 3H), 3.70-3.57 (m, 4H), 3.52 (dd, J=8.5, 5.9 Hz, 1H), 3.45 (s, 3H), 2.94-2.84 (m, 1H), 2.69-2.58 (m, 1H), 2.37-2.24 (m, 2H), 2.10-1.94 (m, 6H), 1.89-1.69 (m, 4H), 1.65-1.44 (m, 2H). LCMS (ESI) calcd. for C₃₀H₃₅F₅N₇O₃S [M+H]⁺ m/z 668.25, found 668.20.

### Example 191

### (R)-2-(4,4-difluoroazepan-1-yl)-N-(3-(N-(2-hydroxyacetyl)-S-methylsulfonimidoyl)phenyl)-4-methyl-5-(1-methyl-1H-pyrazol-4-yl)-6-(trifluoromethyl)nicotinamide

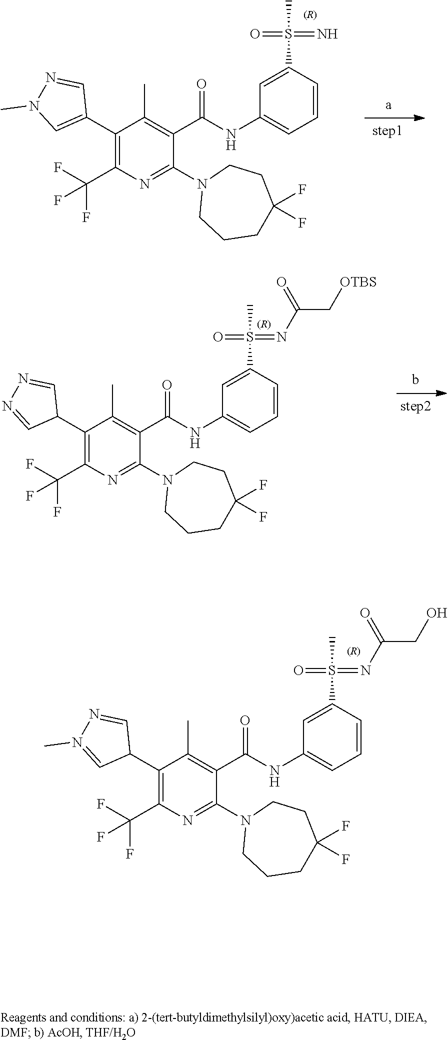

Step 1: (R)-N-(3-(N-(2-((tert-butyldimethylsilyl)oxy)acetyl)-S-methylsulfonimidoyl)phenyl)-2-(4,4-difluoroazepan-1-yl)-4-methyl-5-(1-methyl-1H-pyrazol-4-yl)-6-(trifluoromethyl)nicotinamide: A mixture of (R)-2-(4,4-difluoroazepan-1-yl)-4-methyl-5-(1-methyl-1H-pyrazol-4-yl)-N-(3-(S-methylsulfonimidoyl)phenyl)-6-(trifluoromethyl)nicotinamide (100 mg, 0.20 mmol) and 2-((tert-butyldimethylsilyl)oxy)acetic acid (78 mg, 0.41 mmol) in DMF (5 mL) was added HATU (116 mg, 0.31 mmol) and DIEA (79 mg, 0.61 mmol). The mixture was stirred at room temperature for 16 hours. The reaction was monitored by LCMS. After the reaction was completed, the resulting solution was diluted with water (50 mL) and extracted with DCM (20 mL×3). The combined organic phases were washed with brine, dried over Na₂SO₄, concentrated under vacuum. The residue was purified by flash column chromatography on silica gel (PE/EtOAc=2/1 to 1/1) to give (R)-N-(3-(N-(2-((tert-butyldimethylsilyl)oxy)acetyl)-S-methylsulfonimidoyl)phenyl)-2-(4,4-difluoroazepan-1-yl)-4-methyl-5-(1-methyl-1H-pyrazol-4-yl)-6-(trifluoromethyl)nicotinamide (80 mg, 52.2%) as a yellow solid. LCMS (ESI) calcd. for C₃₃H₄₄F₅N₆O₄SSi [M+H]⁺ m/z 743.28, found 743.15.

Step 2: (R)-2-(4,4-difluoroazepan-1-yl)-N-(3-(N-(2-hydroxyacetyl)-S-methylsulfonimidoyl)phenyl)-4-methyl-5-(1-methyl-1H-pyrazol-4-yl)-6-(trifluoromethyl)nicotinamide: A solution of (R)-N-(3-(N-(2-((tert-butyldimethylsilyl)oxy)acetyl)-S-methylsulfonimidoyl)phenyl)-2-(4,4-difluoroazepan-1-yl)-4-methyl-5-(1-methyl-1H-pyrazol-4-yl)-6-(trifluoromethyl)nicotinamide (80 mg, 0.12 mmol) in THF (3 mL) and H₂O (1 mL) was added AcOH (3 mL) at room temperature. The reaction mixture was stirred at room temperature for 2 hours. After the reaction was completed, the solution was diluted with water (30 mL) and extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 95% MeCN/H₂O containing 0.05% NH₃) to obtain (R)-2-(4,4-difluoroazepan-1-yl)-N-(3-(N-(2-hydroxyacetyl)-S-methylsulfonimidoyl)phenyl)-4-methyl-5-(1-methyl-1H-pyrazol-4-yl)-6-(trifluoromethyl)nicotinamide (24.2 mg, 40.1%) as a white solid. ¹H NMR (400 MHz, DMSO-d₆) δ 11.07 (s, 1H), 8.32 (s, 1H), 8.01-7.95 (m, 1H), 7.71-7.65 (m, 3H), 7.36 (s, 1H), 4.79 (t, J=6.3 Hz, 1H), 3.94 (d, J=6.3 Hz, 2H), 3.90 (s, 3H), 3.68 (s, 2H), 3.61 (t, J=6.0 Hz, 2H), 3.46 (s, 3H), 2.32 (s, 2H), 2.10-2.00 (m, 5H), 1.84 (d, J=5.7 Hz, 2H). LCMS (ESI) calcd. for C₂₇H₃₀F₅N₆O₄S [M+H]⁺ m/z 629.20, found 629.10.

### Example 192

### 2-(4,4-difluoroazepan-1-yl)-N-(3-((R)-N-((S)-2-hydroxypropanoyl)-S-methylsulfonimidoyl)phenyl)-4-methyl-5-(trifluoromethyl)nicotinamide

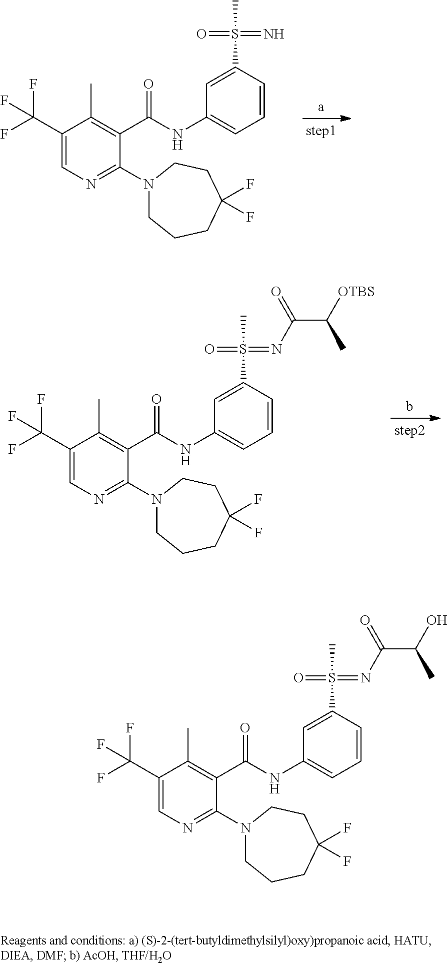 [SMILES: Cc1c(C(F)(F)F)cnc(N2CCCC(F)(F)CC2)c1C(=O)Nc1cccc([S@](C)(=N)=O)c1 ; Cc1c(C(F)(F)F)cnc(N2CCCC(F)(F)CC2)c1C(=O)Nc1cccc([S@@](C)(=O)=NC(=O)[C@H](C)O[Si](C)(C)C(C)(C)C)c1 ; Cc1c(C(F)(F)F)cnc(N2CCCC(F)(F)CC2)c1C(=O)Nc1cccc([S@@](C)(=O)=NC(=O)[C@H](C)O)c1]

Step 1: N-(3-((R)-N-((S)-2-((tert-butyldimethylsilyl)oxy)propanoyl)-S-methylsulfonimidoyl)phenyl)-2-(4,4-difluoroazepan-1-yl)-4-methyl-5-(trifluoromethyl)nicotinamide: A mixture of (R)-2-(4,4-difluoroazepan-1-yl)-4-methyl-N-(3-(S-methylsulfonimidoyl)phenyl)-5-(trifluoromethyl)nicotinamide(100 mg, 0.20 mmol) and (S)-2-((tert-butyldimethylsilyl)oxy)propanoic acid (208 mg, 1.02 mmol) in DMF (5 mL) was added HATU (101 mg, 0.26 mmol) and DIEA (84 mg, 0.64 mmol). The mixture was stirred at room temperature for 4 hours. The reaction was monitored by LCMS. After the reaction was completed, the resulting solution was diluted with water (20 mL) and extracted with DCM (20 mL×3). The combined organic phases were washed with brine, dried over Na₂SO₄, concentrated under vacuum. The residue was purified by flash column chromatography on silica gel (PE/EtOAc=2/1 to 1/1) to give N-(3-((R)-N-((S)-2-((tert-butyldimethylsilyl)oxy)propanoyl)-S-methylsulfonimidoyl)phenyl)-2-(4,4-difluoroazepan-1-yl)-4-methyl-5-(trifluoromethyl)nicotinamide (60 mg, 44.44%) as a white oil. LCMS (ESI) calcd. for C₃₀H₄₂F₅N₄O₄SSi [M+H]⁺ m/z 677.26, found 677.05.

Step 2: 2-(4,4-difluoroazepan-1-yl)-N-(3-((R)-N-((S)-2-hydroxypropanoyl)-S-methylsulfonimidoyl)phenyl)-4-methyl-5-(trifluoromethyl)nicotinamide: A solution of N-(3-((R)-N-((S)-2-((tert-butyldimethylsilyl)oxy)propanoyl)-S-methylsulfonimidoyl)phenyl)-2-(4,4-difluoroazepan-1-yl)-4-methyl-5-(trifluoromethyl)nicotinamide (60 mg, 0.09 mmol) in THE (0.5 mL) was added AcOH (1.5 mL) and H₂O (1.5 mL) at room temperature. The reaction mixture was stirred at room temperature for 2 hours. After the reaction was completed, the solution was diluted with water (10 mL) and extracted with DCM (10 mL×3). The combined organic layers were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.1% formic acid) to obtain 2-(4,4-difluoroazepan-1-yl)-N-(3-((R)-N-((S)-2-hydroxypropanoyl)-S-methylsulfonimidoyl)phenyl)-4-methyl-5-(trifluoromethyl)nicotinamide (35.60 mg, 70.2%) as a white solid. ¹H NMR (400 MHz, DMSO-d₆) δ 11.04 (s, 1H), 8.46 (s, 1H), 8.36 (s, 1H), 7.96-7.93 (m, 1H), 7.69-7.68 (m, 2H), 4.71 (d, J=5.6 Hz, 1H), 4.05-4.02 (m, 1H), 3.74-3.71 (m, 2H), 3.62-3.60 (m, 2H), 3.46 (s, 3H), 2.34-2.30 (m, 5H), 2.07-1.96 (m, 2H), 1.85-1.83 (m, 2H)), 1.25 (d, J=6.8 Hz, 3H). LCMS (ESI) calcd. for C₂₄H₂₈F₅N₄O₄S [M+H]⁺ m/z 563.18, found 563.05.

### Example 193

### 2-(4,4-difluoroazepan-1-yl)-N-(3-((R)-N-((R)-2-hydroxypropanoyl)-S-methylsulfonimidoyl)phenyl)-4-methyl-5-(trifluoromethyl)nicotinamide

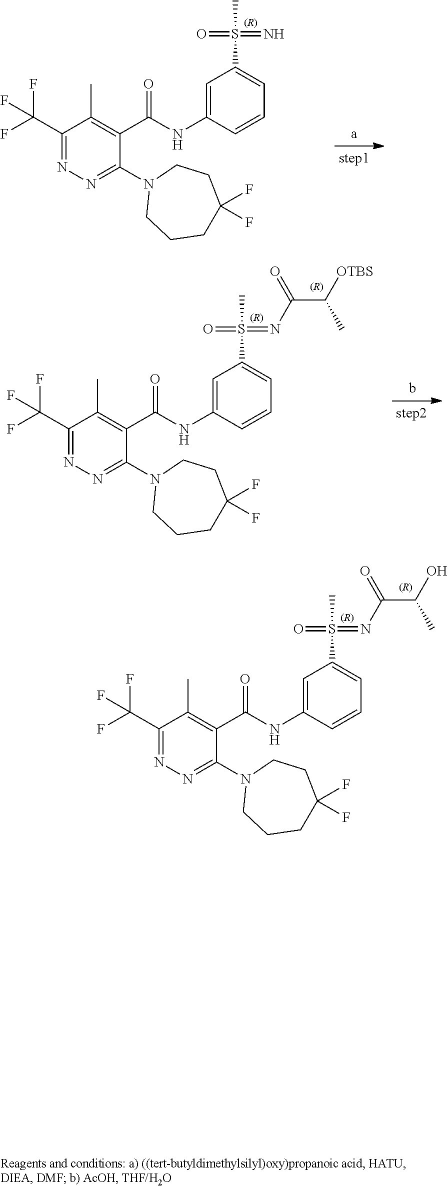

Step 1: N-(3-((R)-N-((R)-2-((tert-butyldimethylsilyl)oxy)propanoyl)-S-methylsulfonimidoyl)phenyl)-2-(4,4-difluoroazepan-1-yl)-4-methyl-5-(trifluoromethyl)nicotinamide: A mixture of (R)-2-(4,4-difluoroazepan-1-yl)-4-methyl-N-(3-(S-methylsulfonimidoyl)phenyl)-5-(trifluoromethyl)nicotinamide (100 mg, 0.20 mmol) and (R)-2-((tert-butyldimethylsilyl)oxy)propanoic acid (208 mg, 1.02 mmol) in DMF (5 mL) was added HATU (101 mg, 0.26 mmol) and DIEA (84 mg, 0.64 mmol). The mixture was stirred at room temperature for 4 hours. The reaction was monitored by LCMS. After the reaction was completed, the resulting solution was diluted with water (20 mL) and extracted with DCM (20 mL×3). The combined organic layers were washed with brine, dried over Na₂SO₄, concentrated under vacuum. The residue was purified by flash column chromatography on silica gel (PE/EtOAc=2/1 to 1/1) to give N-(3-((R)-N-((R)-2-((tert-butyldimethylsilyl)oxy)propanoyl)-S-methylsulfonimidoyl)phenyl)-2-(4,4-difluoroazepan-1-yl)-4-methyl-5-(trifluoromethyl)nicotinamide (60 mg, 44.44%) as a white oil. LCMS (ESI) calcd. for C₃₀H₄₂F₅N₄O₄SSi [M+H]⁺ m/z 677.26, found 677.10.

Step 2: 2-(4,4-difluoroazepan-1-yl)-N-(3-((R)-N-((R)-2-hydroxypropanoyl)-S-methylsulfonimidoyl)phenyl)-4-methyl-5-(trifluoromethyl)nicotinamide. A solution of N-(3-((R)-N-((R)-2-((tert-butyldimethylsilyl)oxy)propanoyl)-S-methylsulfonimidoyl)phenyl)-2-(4,4-difluoroazepan-1-yl)-4-methyl-5-(trifluoromethyl)nicotinamide (60 mg, 0.09 mmol) in THE (0.5 mL) was added AcOH (1.5 mL) and H₂O (1.5 mL) at room temperature. The reaction mixture was stirred at room temperature for 2 hours. After the reaction was completed, the solution was diluted with water (10 mL) and extracted with DCM (10 mL×3). The combined organic layers were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.1% formic acid) to obtain 2-(4,4-difluoroazepan-1-yl)-N-(3-((R)-N-((R)-2-hydroxypropanoyl)-S-methylsulfonimidoyl)phenyl)-4-methyl-5-(trifluoromethyl)nicotinamide (17.80 mg, 35.6%) as a white solid. 1H NMR (400 MHz, DMSO-d₆) δ 11.04 (s, 1H), 8.46 (s, 1H), 8.34-8.33 (m, 1H), 7.98-7.95 (m, 1H), 7.71-7.65 (m, 2H), 4.73 (d, J=5.2 Hz, 1H), 4.04-4.01 (m, 1H), 3.74-3.71 (m, 2H), 3.62-3.60 (m, 2H), 3.47 (s, 3H), 2.32-2.30 (m, 5H), 2.03-2.00 (m, 2H), 1.84-1.82 (m, 2H), 1.27 (d, J=6.8 Hz, 3H). LCMS (ESI) calcd. for C₂₄H₂₈F₅N₄O₄S [M+H]⁺ m/z 563.18, found 563.00.

### Example 194

### (R)-2-(4,4-difluoroazepan-1-yl)-4-methyl-N-(3-(S-methyl-N-(methylglycyl)sulfonimidoyl)phenyl)-5-(trifluoromethyl)nicotinamide

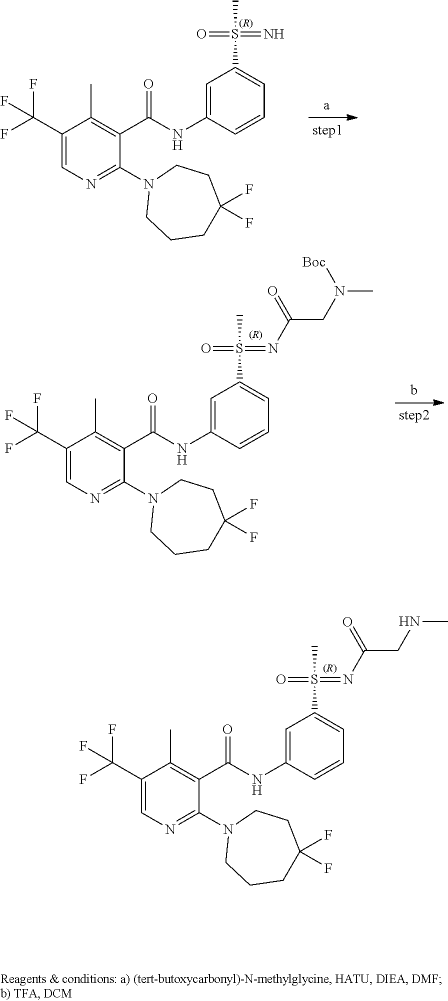

Step 1: tert-butyl (R)-(2-(((3-(2-(4,4-difluoroazepan-1-yl)-4-methyl-5-(trifluoromethyl)nicotinamido)phenyl)(methyl)(oxo)-λ⁶-sulfaneylidene)amino)-2-oxoethyl)(methyl)carbamate: A mixture of (R)-2-(4,4-difluoroazepan-1-yl)-4-methyl-N-(3-(S-methylsulfonimidoyl)phenyl)-5-(trifluoromethyl)nicotinamide (80 mg, 0.16 mmol) and N-(tert-butoxycarbonyl)-N-methylglycine (50 mg, 0.28 mmol) in DMF (5 mL) was added HATU (81 mg, 0.21 mmol) and DIEA (68 mg, 0.51 mmol). The mixture was stirred at room temperature for 4 hours. The reaction was monitored by LCMS. After the reaction was completed, the resulting solution was diluted with water (20 mL) and extracted with DCM (20 mL×3). The combined organic layers were washed with brine, dried over Na₂SO₄, concentrated under vacuum. The residue was purified by flash column chromatography on silica gel (PE/EtOAc=2/1 to 1/1) to give tert-butyl (R)-(2-(((3-(2-(4,4-difluoroazepan-1-yl)-4-methyl-5-(trifluoromethyl)nicotinamido)phenyl)(methyl)(oxo)-λ⁶-sulfaneylidene)amino)-2-oxoethyl)(methyl)carbamate (40 mg, 39.21%) as a white oil. LCMS (ESI) calcd. for C₂₉H₃₇F₅N₅O₅S [M+H]⁺ m/z 662.25, found 662.10.

Step 2: (R)-2-(4,4-difluoroazepan-1-yl)-4-methyl-N-(3-(S-methyl-N-(methylglycyl)sulfonimidoyl)phenyl)-5-(trifluoromethyl)nicotinamide. A solution of tert-butyl (R)-(2-(((3-(2-(4,4-difluoroazepan-1-yl)-4-methyl-5-(trifluoromethyl)nicotinamido)phenyl)(methyl)(oxo)-λ⁶-sulfaneylidene)amino)-2-oxoethyl)(methyl)carbamate (40 mg, 0.06 mmol) in DCM (5 mL) was added TFA (0.5 mL) at room temperature. The reaction mixture was stirred at room temperature for 2 hours. After the reaction was completed, the solution was diluted with water (10 mL) and extracted with DCM (10 mL×3). The combined organic layers were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.1% NH₄OH) to obtain (R)-2-(4,4-difluoroazepan-1-yl)-4-methyl-N-(3-(S-methyl-N-(methylglycyl)sulfonimidoyl)phenyl)-5-(trifluoromethyl)nicotinamide (5.30 mg, 15.15%) as a white solid. ¹H NMR (400 MHz, DMSO-d₆) δ 11.03 (s, 1H), 8.46 (s, 1H), 8.36-8.33 (m, 1H), 7.98-7.96 (m, 1H), 7.71-7.65 (m, 2H), 3.74-3.71 (m, 2H), 3.62-3.60 (m, 2H), 3.46 (s, 3H), 3.21 (s, 2H), 2.34-2.30 (m, 5H), 2.24 (s, 3H), 2.02-1.98 (m, 2H), 1.85-1.81 (m, 2H). LCMS (ESI) calcd. for C₂₄H₂₉F₅N₅O₃S [M+H]⁺ m/z 562.19, found 562.10.

### Example 195

### (R)-2-(4,4-difluoroazepan-1-yl)-N-(3-(N-(2-hydroxyacetyl)-S-methylsulfonimidoyl)phenyl)-4-methyl-5-(trifluoromethyl)nicotinamide

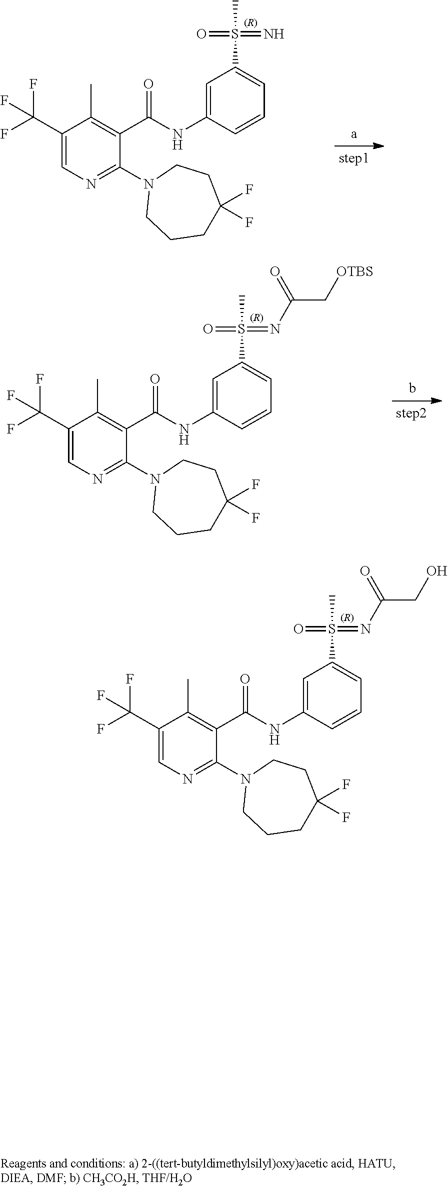

Step 1: (R)-N-(3-(N-(2-((tert-butyldimethylsilyl)oxy)acetyl)-S-methylsulfonimidoyl)phenyl)-2-(4,4-difluoroazepan-1-yl)-4-methyl-5-(trifluoromethyl)nicotinamide: A mixture of (R)-2-(4,4-difluoroazepan-1-yl)-4-methyl-N-(3-(S-methylsulfonimidoyl)phenyl)-5-(trifluoromethyl)nicotinamide (90 mg, 0.18 mmol) and 2-((tert-butyldimethylsilyl)oxy)acetic acid (175 mg, 0.92 mmol) in DMF (5 mL) was added HATU (91 mg, 0.24 mmol) and DIEA (76 mg, 0.59 mmol). The mixture was stirred at room temperature for 4 hours. The reaction was monitored by LCMS. After the reaction was completed, the resulting solution was diluted with water (15 mL) and extracted with DCM (15 mL×3). The combined organic layers were washed with brine, dried over Na₂SO₄, concentrated under vacuum. The residue was purified by flash column chromatography on silica gel (PE/EtOAc=2/1 to 1/1) to give (R)-N-(3-(N-(2-((tert-butyldimethylsilyl)oxy)acetyl)-S-methylsulfonimidoyl)phenyl)-2-(4,4-difluoroazepan-1-yl)-4-methyl-5-(trifluoromethyl)nicotinamide (60 mg, 50.42%) as white oil. LCMS (ESI) calcd. for C₂₉H₄₀F₅N₄O₄SSi [M+H]⁺ m/z 663.25, found 663.10.

Step 2: (R)-2-(4,4-difluoroazepan-1-yl)-N-(3-(N-(2-hydroxyacetyl)-S-methylsulfonimidoyl)phenyl)-4-methyl-5-(trifluoromethyl)nicotinamide: A solution of (R)-N-(3-(N-(2-((tert-butyldimethylsilyl)oxy)acetyl)-S-methylsulfonimidoyl)phenyl)-2-(4,4-difluoroazepan-1-yl)-4-methyl-5-(trifluoromethyl)nicotinamide (60 mg, 0.09 mmol) in THE (0.5 mL) was added CH₃CO₂H (1.5 mL) and H₂O (1.5 mL) at room temperature. The reaction mixture was stirred at room temperature for 2 hours. After the reaction was completed, the solution was diluted with water (10 mL) and extracted with DCM (10 mL×3). The combined organic layers were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.1% formic acid) to obtain (R)-2-(4,4-difluoroazepan-1-yl)-N-(3-(N-(2-hydroxyacetyl)-S-methylsulfonimidoyl)phenyl)-4-methyl-5-(trifluoromethyl)nicotinamide (11.30 mg, 22.9%) as a white solid. ¹H NMR (400 MHz, DMSO-d₆) δ 11.02 (s, 1H), 8.46 (s, 1H), 8.32-8.29 (m, 1H), 8.01-7.99 (m, 1H), 7.71-7.67 (m, 2H), 4.79-4.76 (m, 1H), 3.94 (d, J=6.4 Hz, 2H), 3.73-3.71 (m, 2H), 3.64-3.61 (m, 2H), 3.47 (s, 3H), 2.34-2.30 (m, 5H), 2.03-2.00 (m, 2H), 1.85-1.83 (m, 2H). LCMS (ESI) calcd. for C₂₃H₂₆F₅N₄O₄S [M+H]⁺ m/z 549.16, found 549.05.

### Example 196

### (R)-N-(3-(N-(2-aminoethyl)-S-methylsulfonimidoyl)phenyl)-5-cyano-2-(4,4-difluoroazepan-1-yl)-4-methyl-6-(trifluoromethyl)nicotinamide

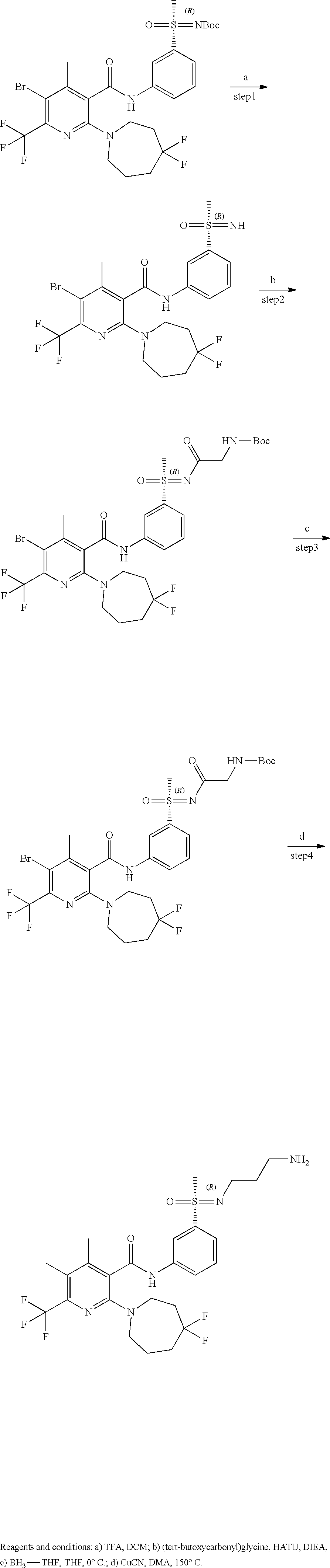

Step 1: (R)-5-bromo-2-(4,4-difluoroazepan-1-yl)-4-methyl-N-(3-(S-methylsulfonimidoyl)phenyl)-6-(trifluoromethyl)nicotinamide: A solution of tert-butyl (R)-((3-(5-bromo-2-(4,4-difluoroazepan-1-yl)-4-methyl-6-(trifluoromethyl)nicotinamido)phenyl)(methyl)(oxo)-λ⁶-sulfaneylidene)carbamate (500 mg, 0.75 mmol) in DCM (6 mL) was added TFA (0.6 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum to provide (R)-5-bromo-2-(4,4-difluoroazepan-1-yl)-4-methyl-N-(3-(S-methylsulfonimidoyl)phenyl)-6-(trifluoromethyl)nicotinamide (400 mg, 94.2%) as a yellow solid. LCMS (ESI) calcd. for C₂₁H₂₃BrF₅N₄O₂S [M+H]⁺ m/z 569.07, found 570.75.

Step 2: tert-butyl (R)-(2-(((3-(5-bromo-2-(4,4-difluoroazepan-1-yl)-4-methyl-6-(trifluoromethyl)nicotinamido)phenyl)(methyl)(oxo)-λ⁶-sulfaneylidene)amino)-2-oxoethyl)carbamate: A solution of (R)-5-bromo-2-(4,4-difluoroazepan-1-yl)-4-methyl-N-(3-(S-methylsulfonimidoyl)phenyl)-6-(trifluoromethyl)nicotinamide (400 mg, 0.70 mmol), (tert-butoxycarbonyl)glycine (248 mg, 1.41 mmol), HATU (401 mg, 1.05 mmol) and DIEA (272 mg, 2.11 mmol) in DMF (10 mL) was stirred at room temperature for 16 hours. LCMS showed the reaction was completed. The mixture was diluted with water (30 mL) and extracted with EtOAc (30 mL×3). The combined organic layers were washed with brine, dried over sodium sulfate, concentrated under vacuum. The residue was purified by flash column chromatography on silica gel (PE/EtOAc=3/1) to provide tert-butyl (R)-(2-(((3-(5-bromo-2-(4,4-difluoroazepan-1-yl)-4-methyl-6-(trifluoromethyl)nicotinamido)phenyl)(methyl)(oxo)-λ⁶-sulfaneylidene)amino)-2-oxoethyl)carbamate (410 mg, 72.3%) as yellow solid. LCMS (ESI) calcd. for C₂₈H₃₄BrF₅N₅O₅S [M+H]⁺ m/z 726.14, found 727.95.

Step 3: tert-butyl (R)-(2-(((3-(5-bromo-2-(4,4-difluoroazepan-1-yl)-4-methyl-6-(trifluoromethyl)nicotinamido)phenyl)(methyl)(oxo)-λ⁶-sulfaneylidene)amino)ethyl)carbamate: A solution of tert-butyl (R)-(2-(((3-(5-bromo-2-(4,4-difluoroazepan-1-yl)-4-methyl-6-(trifluoromethyl)nicotinamido)phenyl)(methyl)(oxo)-λ⁶-sulfaneylidene)amino)-2-oxoethyl)carbamate (400 mg, 0.55 mmol) in THE (10 mL) was added BH₃-THF (1.5 mL) at 0° C. The reaction mixture was stirred at 0° C. for 1 hour. LCMS showed the reaction was completed. The mixture was diluted with water (30 mL) and extracted with EtOAc (30 mL×3). The combined organic layers were washed with brine, dried over sodium sulfate, concentrated under vacuum. The residue was purified by flash column chromatography on silica gel (PE/EtOAc=3/1) to provide tert-butyl (R)-(2-(((3-(5-bromo-2-(4,4-difluoroazepan-1-yl)-4-methyl-6-(trifluoromethyl)nicotinamido)phenyl)(methyl)(oxo)-λ⁶-sulfaneylidene)amino)ethyl)carbamate (160 mg, 40.8%) as a yellow solid. LCMS (ESI) calcd. for C₂₈H₃₆BrF₅N₅O₄S [M+H]⁺ m/z 712.16, found 712.00.

Step 4: (R)-N-(3-(N-(2-aminoethyl)-S-methylsulfonimidoyl)phenyl)-5-cyano-2-(4,4-difluoroazepan-1-yl)-4-methyl-6-(trifluoromethyl)nicotinamide. A mixture of tert-butyl (R)-(2-(((3-(5-bromo-2-(4,4-difluoroazepan-1-yl)-4-methyl-6-(trifluoromethyl)nicotinamido)phenyl)(methyl)(oxo)-λ⁶-sulfaneylidene)amino)ethyl)carbamate (160 mg, 0.22 mmol), CuCN (201 mg, 2.24 mmol) in DMA (10 mL) was heated to 150° C. and stirred for 2 hours under an atmosphere of N₂. After cooling to ambient temperature, the mixture was filtered through celite and the filtrate was concentrated under vacuum. The mixture was diluted with water (30 mL) and extracted with EtOAc (30 mL×3). The combined organic layers were washed with brine, dried over sodium sulfate, concentrated under vacuum. The residue was purified by prep-HPLC (Gemini 5 um C₁₈ column, 150*21.2 mm, eluting with 35% to 40% MeCN/H₂O containing 0.1% formic acid) to provide (R)-N-(3-(N-(2-aminoethyl)-S-methylsulfonimidoyl)phenyl)-5-cyano-2-(4,4-difluoroazepan-1-yl)-4-methyl-6-(trifluoromethyl)nicotinamide (22.3 mg, 17.3%) as a white solid. ¹H NMR (400 MHz, DMSO-d₆) δ 11.38 (s, 1H), 8.23 (s, 1H), 8.01-7.91 (m, 1H), 7.66 (d, J=4.9 Hz, 2H), 3.84-3.63 (m, 4H), 3.19 (s, 3H), 3.09-2.69 (m, 4H), 2.47 (s, 3H), 2.36-2.24 (m, 2H), 2.11-1.95 (m, 2H), 1.93-1.80 (m, 2H). ¹H NMR (400 MHz, CD₃OD) δ 8.52 (s, 1H), 8.41 (s, 1H), 7.87 (d, J=8.0 Hz, 1H), 7.77-7.65 (m, 2H), 3.90-3.72 (m, 4H), 3.24 (s, 3H), 3.22-3.07 (m, 2H), 3.05 (d, J=4.9 Hz, 2H), 2.55 (s, 3H), 2.39-2.27 (m, 2H), 2.09-1.91 (m, 4H). LCMS (ESI) calcd. for C₂₄H₂₈F₅N₆O₂S [M+H]⁺ m/z 559.19, found 559.00.

### Example 197

### (R)-5-(4,4-difluoroazepan-1-yl)-3-methyl-N-(3-(S-methylsulfonimidoyl)phenyl)-2-(trifluoromethyl)isonicotinamide

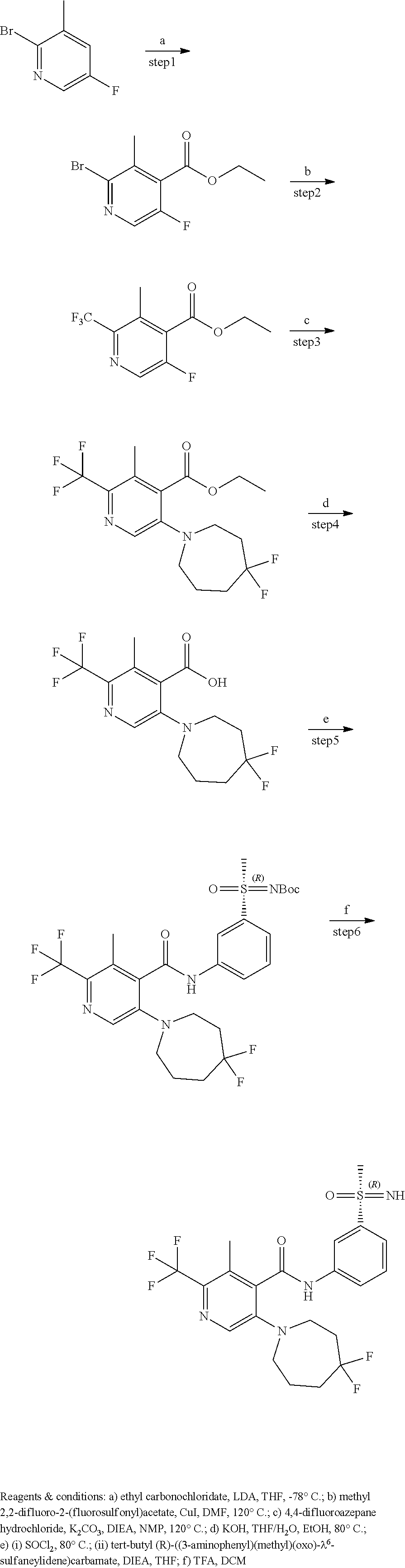

Step 1: ethyl 2-bromo-5-fluoro-3-methylisonicotinate: A solution of 2-bromo-5-fluoro-3-methylpyridine (4 g, 21.17 mmol) in THE (30 mL) at −78° C. under an atmosphere of N₂ was added dropwise a solution of LDA (19.7 mL). The mixture was stirred at −78° C. for 1 hour. Then a solution of ethyl carbonochloridate (6.87 g, 63.31 mmol) in THE (10 mL) was added dropwise. The reaction mixture was stirred at 25° C. for 2 hours. After the reaction was completed, the reaction was quenched with saturated NH₄Cl (80 mL) and extracted with EtOAc (100 mL×3). The combined organic layers were dried over sodium sulfate and evaporated to give a residue, which was purified by flash column chromatography on silica gel (PE/EtAOc=10/1) to give ethyl 2-bromo-5-fluoro-3-methylisonicotinate (1.2 g, 21.9%) as white oil. LCMS (ESI) calcd. for C₉H₁₀BrFNO₂ [M+H]⁺ m/z 261.99, found 261.80.

Step 2: ethyl 5-fluoro-3-methyl-2-(trifluoromethyl)isonicotinate: A mixture of ethyl 2-bromo-5-fluoro-3-methylisonicotinate (1.2 g, 4.58 mmol), methyl 2,2-difluoro-2-(fluorosulfonyl)acetate (4.42 g, 23.01 mmol) and CuI (1.75 g, 9.19 mmol) in DMF (15 mL) was heated to 120° C. and refluxed for 5 hours. After the reaction was completed, the mixture was cooled to room temperature. The resulting solution was diluted with EtOAc (50 mL) and washed with water (50 mL×3). The combined organic layers were washed with brine, dried over sodium sulfate, concentrated under vacuum. The residue was purified by flash column chromatography on silica gel (PE/EtOAc=10/1) to provide ethyl 5-fluoro-3-methyl-2-(trifluoromethyl)isonicotinate (950 mg, 82.3%) as white oil. LCMS (ESI) calcd. for C₁₀H₁₀F₄NO₂ [M+H]⁺ m/z 252.07, found 251.60.

Step 3: ethyl 5-(4,4-difluoroazepan-1-yl)-3-methyl-2-(trifluoromethyl)isonicotinate: A solution of ethyl 5-fluoro-3-methyl-2-(trifluoromethyl)isonicotinate (950 mg, 3.78 mmol), 4,4-difluoroazepane hydrochloride (1.3 g, 7.57 mmol), K₂CO₃ (3.1 g, 22.46 mmol) and DIEA (2.9 g, 22.44 mmol) in NMP (15 mL) was heated to 120° C. and refluxed for 16 hours. After the reaction was completed, the mixture was cooled to room temperature. The resulting solution was diluted with EtOAc (50 mL) and washed with water (50 mL×3). The combined organic layers were washed with brine, dried over sodium sulfate, concentrated under vacuum. The residue was purified by flash column chromatography on silica gel (PE/EtOAc=1/1) to provide ethyl 5-(4,4-difluoroazepan-1-yl)-3-methyl-2-(trifluoromethyl)isonicotinate (500 mg, 36%) as a white solid. LCMS (ESI) calcd. for C₁₆H₂₀F₅N₂O₂ [M+H]⁺ m/z 367.15, found 366.95.

Step 4: 5-(4,4-difluoroazepan-1-yl)-3-methyl-2-(trifluoromethyl)isonicotinic acid: A solution of ethyl 5-(4,4-difluoroazepan-1-yl)-3-methyl-2-(trifluoromethyl)isonicotinate (450 mg, 1.23 mmol) in THF/H₂O/EtOH (1/1/1, 12 mL) was added KOH (689 mg, 12.28 mmol) at room temperature. The reaction mixture was heated to 80° C. and refluxed for 16 hours. After the reaction was completed, the residue was adjusted to pH4-5 with 3M HCl. Then the aqueous solution was extracted with DCM (30 mL×3). The combine organic layers were washed with brine, dried over Na₂SO₄, concentrated under vacuum to give 5-(4,4-difluoroazepan-1-yl)-3-methyl-2-(trifluoromethyl)isonicotinic acid (400 mg, 98.6%) as a white solid. LCMS (ESI) calcd. for C₁₄H₁₆F₅N₂O₂ [M+H]⁺ m/z 339.12, found 338.95.

Step 5: tert-butyl (R)-((3-(5-(4,4-difluoroazepan-1-yl)-3-methyl-2-(trifluoromethyl)isonicotinamido)phenyl)(methyl)(oxo)-λ⁶-sulfaneylidene)carbamate: A solution of 5-(4,4-difluoroazepan-1-yl)-3-methyl-2-(trifluoromethyl)isonicotinic acid (200 mg, 0.59 mmol) in SOCl₂ (5 mL) was heated to 80° C. and stirred for 2 hours. The solution was concentrated under vacuum to provide the chloride intermediate. Then the chloride intermediate dissolved in THE (2 mL) was added to a solution of tert-butyl (R)-((3-aminophenyl)(methyl)(oxo)-λ⁶-sulfaneylidene)carbamate (240 mg, 0.89 mmol) and DIEA (382 mg, 2.96 mmol) in THE (3 mL) at 0° C. The resulting mixture was stirred at 25° C. for 16 hours. Then the mixture was quenched with water (20 mL) and extracted with DCM (20 mL×3). The combined organic layers were washed with brine, dried over sodium sulfate, concentrated under vacuum. The residue was purified by flash column chromatography on silica gel (PE/EtOAc=1/1) to provide tert-butyl (R)-((3-(5-(4,4-difluoroazepan-1-yl)-3-methyl-2-(trifluoromethyl)isonicotinamido)phenyl)(methyl)(oxo)-λ⁶-sulfaneylidene)carbamate (160 mg, 46% yield) as white oil. LCMS (ESI) calcd. for C₂₆H₃₂F₅N₄O₄S [M+H]⁺ m/z 591.21, found 591.10.

Step 6: (R)-5-(4,4-difluoroazepan-1-yl)-3-methyl-N-(3-(S-methylsulfonimidoyl)phenyl)-2-(trifluoromethyl)isonicotinamide: A solution of tert-butyl (R)-((3-(5-(4,4-difluoroazepan-1-yl)-3-methyl-2-(trifluoromethyl)isonicotinamido)phenyl)(methyl)(oxo)-λ⁶-sulfaneylidene)carbamate (160 mg, 0.27 mmol) in DCM (5 mL) was added TFA (1 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (15 mL×3). The combined organic layers were washed with brine, dried over sodium sulfate, concentrated under vacuum. The residue was purified by prep-HPLC (Gemini 5 um C₁₈ column, 150*21.2 mm, eluting with 40% to 90% MeCN/H₂O containing 0.05% NH₃) to provide (R)-5-(4,4-difluoroazepan-1-yl)-3-methyl-N-(3-(S-methylsulfonimidoyl)phenyl)-2-(trifluoromethyl)isonicotinamide (43.5 mg, 32.9%) as a white solid. ¹H NMR (400 MHz, DMSO-d₆) δ 11.01 (s, 1H), 8.33 (s, 2H), 7.89 (d, J=8.0 Hz, 1H), 7.70 (d, J=7.8 Hz, 1H), 7.61 (t, J=7.9 Hz, 1H), 4.25 (s, 1H), 3.56-3.44 (m, 4H), 3.06 (s, 3H), 2.33 (s, 3H), 2.21 (s, 2H), 2.11-1.99 (m, 2H), 1.81 (d, J=5.5 Hz, 2H). LCMS (ESI) calcd. for C₂₁H₂₄F₅N₄O₂S [M+H]⁺ m/z 491.16, found 491.00.

### Example 198

### (R)-3-(4,4-difluoroazepan-1-yl)-N-(3-(N-(2-(3,3-difluoroazetidin-1-yl)acetyl)-S-methylsulfonimidoyl)phenyl)-5-methyl-6-(trifluoromethyl)pyridazine-4-carboxamide

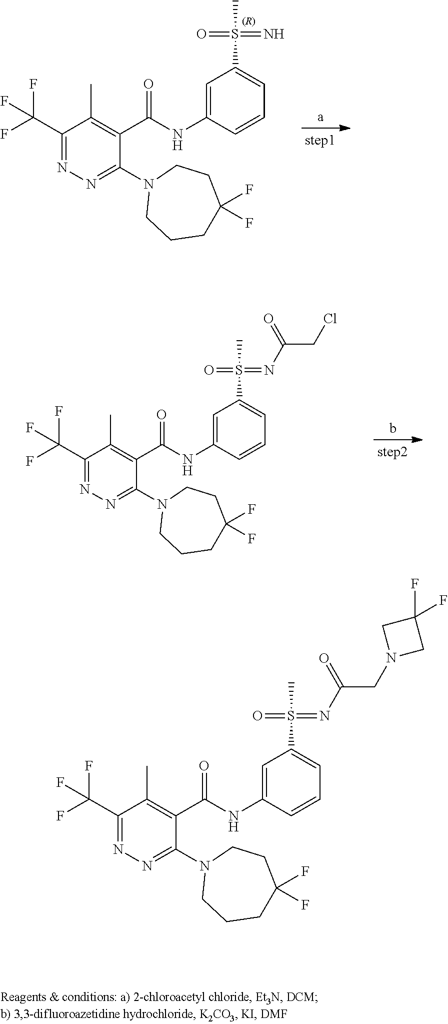

Step 1: (R)-N-(3-(N-(2-chloroacetyl)-S-methylsulfonimidoyl)phenyl)-3-(4,4-difluoroazepan-1-yl)-5-methyl-6-(trifluoromethyl)pyridazine-4-carboxamide: A mixture of (R)-3-(4,4-difluoroazepan-1-yl)-5-methyl-N-(3-(S-methylsulfonimidoyl)phenyl)-6-(trifluoromethyl)pyridazine-4-carboxamide (200 mg, 0.41 mmol) and TEA (124 mg, 1.22 mmol) in DCM (10 mL) was added 2-chloroacetyl chloride (51 mg, 0.45 mmol) at room temperature. The reaction mixture was stirred at room temperature for 16 hours. After the reaction was completed, the resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtAOc=1/1) to provide (R)-N-(3-(N-(2-chloroacetyl)-S-methylsulfonimidoyl)phenyl)-3-(4,4-difluoroazepan-1-yl)-5-methyl-6-(trifluoromethyl)pyridazine-4-carboxamide (150 mg, 64.9%) as a yellow solid. LCMS (ESI) calcd. for C₂₂H₂₄ClF₅N₅O₃S [M+H]⁺ m/z 568.12, found 568.05.

Step 2: (R)-3-(4,4-difluoroazepan-1-yl)-N-(3-(N-(2-(3,3-difluoroazetidin-1-yl)acetyl)-S-methylsulfonimidoyl)phenyl)-5-methyl-6-(trifluoromethyl)pyridazine-4-carboxamide: A mixture of (R)-N-(3-(N-(2-chloroacetyl)-S-methylsulfonimidoyl)phenyl)-3-(4,4-difluoroazepan-1-yl)-5-methyl-6-(trifluoromethyl)pyridazine-4-carboxamide (140 mg, 0.25 mmol), KI (82 mg, 0.50 mmol) and K₂CO₃ (68 mg, 0.50 mmol) in DMF (5 mL) was added 3,3-difluoroazetidine hydrochloride (64 mg, 0.50 mmol) at room temperature. The reaction mixture was stirred at room temperature for 16 hours. After the reaction was completed, the resulting solution was diluted with water (15 mL) and extracted with EtOAc (15 mL×3). The combined organic layers were washed with brine, dried over sodium sulfate, concentrated under vacuum. The residue was purified by prep-HPLC (Gemini 5 um C₁₈ column, 150*21.2 mm, eluting with 40% to 85% MeCN/H₂O containing 0.05% NH₃) to provide (R)-3-(4,4-difluoroazepan-1-yl)-N-(3-(N-(2-(3,3-difluoroazetidin-1-yl)acetyl)-S-methylsulfonimidoyl)phenyl)-5-methyl-6-(trifluoromethyl)pyridazine-4-carboxamide (34.60 mg, 22.1%) as a white solid. ¹H NMR (400 MHz, DMSO-d₆) δ 11.20 (s, 1H), 8.29 (s, 1H), 7.93 (d, J=7.2 Hz, 1H), 7.76-7.66 (m, 2H), 3.85 (s, 2H), 3.71-3.60 (m, 6H), 3.47 (s, 3H), 3.37 (s, 2H), 2.44-2.31 (m, 5H), 2.12-2.01 (m, 2H), 1.94-1.83 (m, 2H). LCMS (ESI) calcd. for C₂₅H₂₈F₇N₆O₃S [M+H]⁺ m/z 625.19, found 625.10.

### Example 199

### (R)-5-chloro-2-(4,4-difluoroazepan-1-yl)-N-(2-(S-methylsulfonimidoyl)pyridin-4-yl)-4-(trifluoromethyl)benzamide

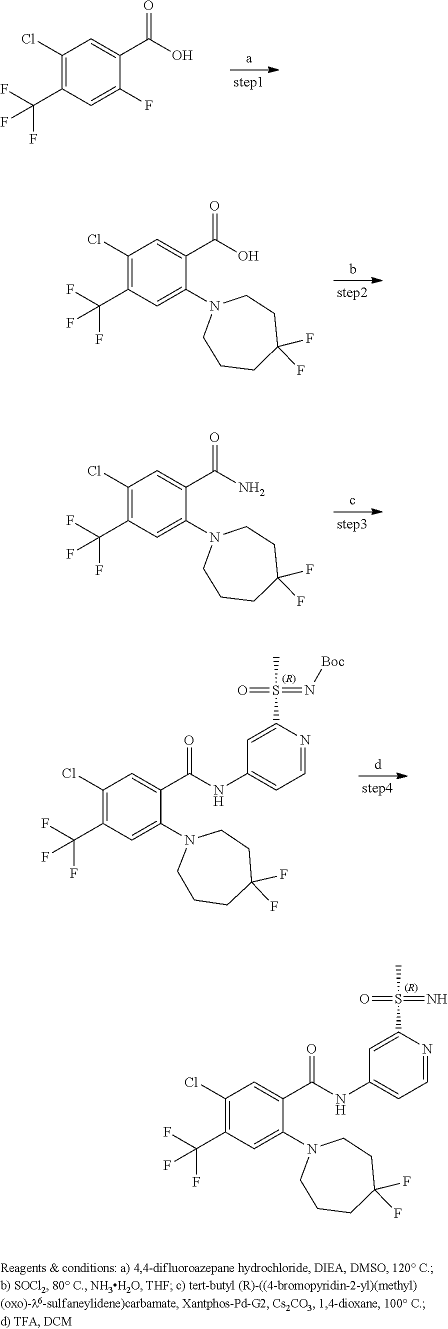

Step 1: 5-chloro-2-(4,4-difluoroazepan-1-yl)-4-(trifluoromethyl)benzoic acid: A solution of 5-chloro-2-fluoro-4-(trifluoromethyl)benzoic acid (300 mg, 1.23 mmol), 4,4-difluoroazepane hydrochloride (380 mg, 2.22 mmol), and DIEA (637 mg, 4.93 mmol) in DMSO (6 mL) was heated to 120° C. and stirred for 16 hours. After the reaction was completed, the mixture was cooled to room temperature. The resulting solution was diluted with EtOAc (50 mL) and washed with water (20 mL×3). The combined organic layers were washed with brine, dried over sodium sulfate, concentrated under vacuum. The residue was purified by flash column chromatography on silica gel (PE/EtOAc=1/1) to provide 5-chloro-2-(4,4-difluoroazepan-1-yl)-4-(trifluoromethyl)benzoic acid (180 mg, 40.7%) as yellow oil. LCMS (ESI) calcd. for C₁₄H₁₄ClF₅NO₂ [M+H]⁺ m/z 358.07, found 358.00.

Step 2: 5-chloro-2-(4,4-difluoroazepan-1-yl)-4-(trifluoromethyl)benzamide: A solution of 5-chloro-2-(4,4-difluoroazepan-1-yl)-4-(trifluoromethyl)benzoic acid (180 mg, 0.50 mmol) in SOCl₂ (5 mL) was heated to 80° C. and stirred for 0.5 hour. The solution was concentrated under vacuum to provide the chloride intermediate. Then the chloride intermediate dissolved in THE (5 mL) was added to a solution of THE (5 mL) in NH₃·H₂O (5 mL) at 0° C. The resulting mixture was stirred at 25° C. for 1 hour. Then the mixture was quenched with water (20 mL) and extracted with DCM (20 mL×3). The combined organic layers were washed with brine, dried over sodium sulfate, concentrated under vacuum. The residue was purified by flash column chromatography on silica gel (PE/EtOAc=1/1) to provide 5-chloro-2-(4,4-difluoroazepan-1-yl)-4-(trifluoromethyl)benzamide (84 mg, 46.8%) as yellow oil. LCMS (ESI) calcd. for C₁₄H₁₅ClF₅N₂O [M+H]⁺ m/z 357.08, found 357.00.

Step 3: tert-butyl (R)-((4-(5-chloro-2-(4,4-difluoroazepan-1-yl)-4-(trifluoromethyl)benzamido)pyridin-2-yl)(methyl)(oxo)-λ⁶-sulfaneylidene)carbamate: A mixture of 5-chloro-2-(4,4-difluoroazepan-1-yl)-4-(trifluoromethyl)benzamide (84 mg, 0.24 mmol), tert-butyl (R)-((4-bromopyridin-2-yl)(methyl)(oxo)-λ⁶-sulfaneylidene)carbamate (79 mg, 0.24 mmol), Cs₂CO₃ (200 mg, 0.61 mmol) and Xantphos-Pd-G2 (42 mg, 0.05 mmol) in 1,4-dioxane (5 mL) was heated to 100° C. and stirred for 2 hours under nitrogen. After the reaction was completed, the mixture was filtered through celite. The filtrate was concentrated and residue was purified by flash column chromatography on silica gel (PE/EtOAc=1/1) to provide tert-butyl (R)-((4-(5-chloro-2-(4,4-difluoroazepan-1-yl)-4-(trifluoromethyl)benzamido)pyridin-2-yl)(methyl)(oxo)-λ⁶-sulfaneylidene)carbamate (110 mg, 91.6%) as yellow solid. LCMS (ESI) calcd. for C₂₅H₂₉ClF₅N₄O₄S [M+H]⁺ m/z 611.15, found 611.10.

Step 4: (R)-5-chloro-2-(4,4-difluoroazepan-1-yl)-N-(2-(S-methylsulfonimidoyl)pyridin-4-yl)-4-(trifluoromethyl)benzamide: A solution of tert-butyl (R)-((4-(5-chloro-2-(4,4-difluoroazepan-1-yl)-4-(trifluoromethyl)benzamido)pyridin-2-yl)(methyl)(oxo)-λ⁶-sulfaneylidene)carbamate (110 mg, 0.18 mmol) in DCM (5 mL) was added TFA (0.5 mL) at room temperature. The reaction mixture was stirred at room temperature for 1 hour. After the reaction was completed, the mixture was concentrated. The residue was adjusted to pH=8-9 with saturated aqueous NaHCO₃. Then the aqueous solution was extracted with DCM (15 mL×3). The combined organic layers were washed with brine, dried over sodium sulfate, concentrated under vacuum. The residue was purified by prep-HPLC (Gemini 5 um C₁₈ column, 150*21.2 mm, eluting with 40% to 90% MeCN/H₂O containing 0.05% NH₃) to provide (R)-5-chloro-2-(4,4-difluoroazepan-1-yl)-N-(2-(S-methylsulfonimidoyl)pyridin-4-yl)-4-(trifluoromethyl)benzamide (35.60 mg, 38.1%) as a white solid. ¹H NMR (400 MHz, DMSO-d₆) δ 11.23 (s, 1H), 8.60 (d, J=4.9 Hz, 1H), 8.38 (s, 1H), 7.82 (d, 1H), 7.72 (s, 1H), 7.34 (s, 1H), 4.33 (s, 1H), 3.44-3.38 (m, 2H), 3.36 (t, J=5.8 Hz, 2H), 3.14 (s, 3H), 2.31-2.15 (m, 2H), 2.12-1.98 (m, 2H), 1.84-1.74 (m, 2H). LCMS (ESI) calcd. for C₂₀H₂₁ClF₅N₄O₂S [M+H]⁺ m/z 511.10, found 511.00.

### Example 200

### (R)-2-(4,4-difluoroazepan-1-yl)-4-methyl-5-(1-methyl-1H-pyrazol-4-yl)-N-(2-(S-methylsulfonimidoyl)pyridin-4-yl)nicotinamide

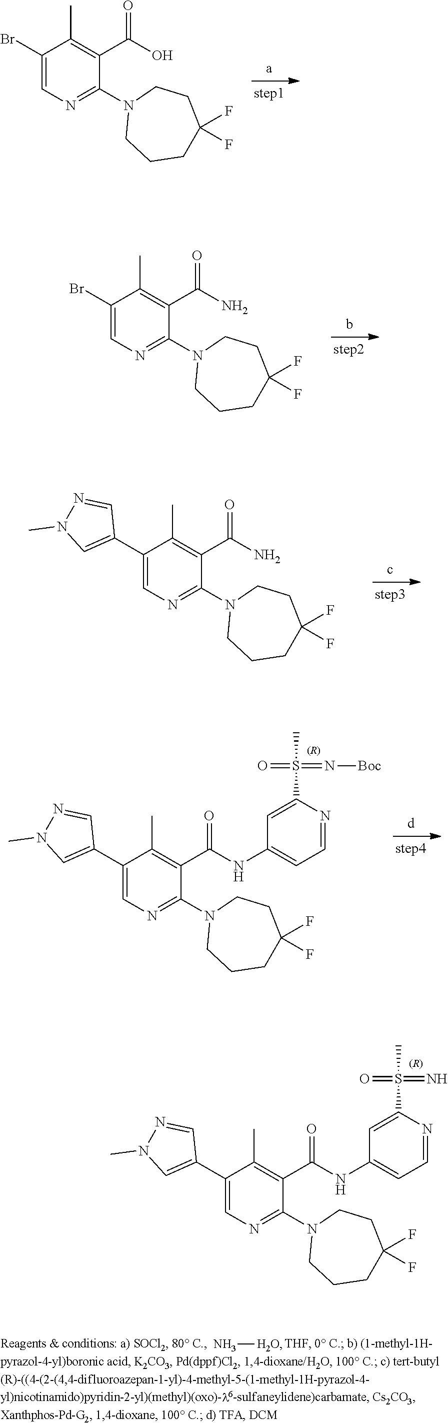

Step 1: 5-bromo-2-(4,4-difluoroazepan-1-yl)-4-methylnicotinamide. A solution of 5-bromo-2-(4,4-difluoroazepan-1-yl)-4-methylnicotinic acid (400 mg, 1.15 mmol) in SOCl₂ (3 mL) was heated to 80° C. and stirred for 2 hours. The solution was concentrated under vacuum to provide the chloride intermediate. Then the chloride intermediate dissolved in THF (2 mL) was added to NH₃·H₂O (6 mL) at 0° C. The resulting mixture was stirred at 25° C. for 2 hours. Then the mixture was quenched with water (30 mL) and extracted with DCM (30 mL×3). The combined organic layers were washed with brine, dried over sodium sulfate, concentrated under vacuum. The residue was purified by flash column chromatography on silica gel (PE/EtOAc=1/1) to provide 5-bromo-2-(4,4-difluoroazepan-1-yl)-4-methylnicotinamide (300 mg, 71.5%) as a yellow solid. LCMS (ESI) calcd. for C₁₃H₁₇BrF₂N₃O [M+H]⁺ m/z 348.05, found 349.85

Step 2: 2-(4,4-difluoroazepan-1-yl)-4-methyl-5-(1-methyl-1H-pyrazol-4-yl)nicotinamide: A solution of 5-bromo-2-(4,4-difluoroazepan-1-yl)-4-methylnicotinamide (300 mg, 0.86 mmol), (1-methyl-1H-pyrazol-4-yl)boronic acid (217 mg, 1.72 mmol), K₂CO₃ (357 mg, 2.59 mmol) and Pd(dppf)Cl₂ (63 mg, 0.09 mmol) in 1,4-dioxane/H₂O (4/1, 10 mL) was heated to 100° C. and refluxed for 16 hours under nitrogen. After the reaction was completed, the mixture was cooled to room temperature. The mixture was quenched with water (30 mL) and extracted with DCM (30 mL×3). The combined organic layers were washed with brine, dried over sodium sulfate, concentrated under vacuum. The residue was purified by flash column chromatography on silica gel (PE/EtOAc=1/1) to provide 2-(4,4-difluoroazepan-1-yl)-4-methyl-5-(1-methyl-1H-pyrazol-4-yl)nicotinamide (200 mg, 63.2%) as a white solid. LCMS (ESI) calcd. for C₁₇H₂₂F₂N₅O [M+H]⁺ m/z 350.18, found 350.05.

Step 3: tert-butyl (R)-((4-(2-(4,4-difluoroazepan-1-yl)-4-methyl-5-(1-methyl-1H-pyrazol-4-yl)nicotinamido)pyridin-2-yl)(methyl)(oxo)-λ⁶-sulfaneylidene)carbamate: A solution of 2-(4,4-difluoroazepan-1-yl)-4-methyl-5-(1-methyl-1H-pyrazol-4-yl)nicotinamide (200 mg, 0.57 mmol), tert-butyl (R)-((4-bromopyridin-2-yl)(methyl)(oxo)-λ⁶-sulfaneylidene)carbamate (289 mg, 0.86 mmol), Cs₂CO₃ (560 mg, 1.72 mmol) and Xantphos-Pd-G2 (51 mg, 0.06 mmol) in 1,4-dioxane (6 mL) was heated to 100° C. and refluxed for 16 hours under nitrogen. After the reaction was completed, the mixture was cooled to room temperature. The mixture was quenched with water (20 mL) and extracted with DCM (20 mL×3). The combined organic layers were washed with brine, dried over sodium sulfate, concentrated under vacuum. The residue was purified by flash column chromatography on silica gel (PE/EtOAc=1/1) to provide tert-butyl (R)-((4-(2-(4,4-difluoroazepan-1-yl)-4-methyl-5-(1-methyl-1H-pyrazol-4-yl)nicotinamido)pyridin-2-yl)(methyl)(oxo)-λ⁶-sulfaneylidene)carbamate (180 mg, 49.5%) as a white solid. LCMS (ESI) calcd. for C₂₈H₃₆F₂N₇O₄S [M+H]⁺ m/z 604.25, found 604.15.

Step 4: (R)-2-(4,4-difluoroazepan-1-yl)-4-methyl-5-(1-methyl-1H-pyrazol-4-yl)-N-(2-(S-methylsulfonimidoyl)pyridin-4-yl)nicotinamide: A solution of tert-butyl (R)-((4-(2-(4,4-difluoroazepan-1-yl)-4-methyl-5-(1-methyl-1H-pyrazol-4-yl)nicotinamido)pyridin-2-yl)(methyl)(oxo)-λ⁶-sulfaneylidene)carbamate (180 mg, 0.30 mmol) in DCM (5 mL) was added TFA (1 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (15 mL×3). The combined organic layers were washed with brine, dried over sodium sulfate, concentrated under vacuum. The residue was purified by prep-HPLC (Gemini 5 um C₁₈ column, 150*21.2 mm, eluting with 30% to 70% MeCN/H₂O containing 0.05% NH₃) to provide (R)-2-(4,4-difluoroazepan-1-yl)-4-methyl-5-(1-methyl-1H-pyrazol-4-yl)-N-(2-(S-methylsulfonimidoyl)pyridin-4-yl)nicotinamide(96.70 mg, 64.1%) as a white solid. ¹H NMR (400 MHz, DMSO-d₆) δ 11.26 (s, 1H), 8.61 (d, J=5.4 Hz, 1H), 8.43 (m, 1H), 8.20 (s, 1H), 7.90 (s, 1H), 7.82 (m, 1H), 7.61 (s, 1H), 4.38 (s, 1H), 3.88 (s, 3H), 3.62-3.48 (m, 4H), 3.15 (s, 3H), 2.33-2.22 (m, 5H), 2.08-1.96 (m, 2H), 1.80 (m, 2H). LCMS (ESI) calcd. for C₂₃H₂₈F₂N₇O₂S [M+H]⁺ m/z 504.20, found 504.10.

### Example 201

### (R)-5-cyano-2-(4,4-difluoroazepan-1-yl)-4-methyl-N-(2-(S-methylsulfonimidoyl)pyridin-4-yl)nicotinamide

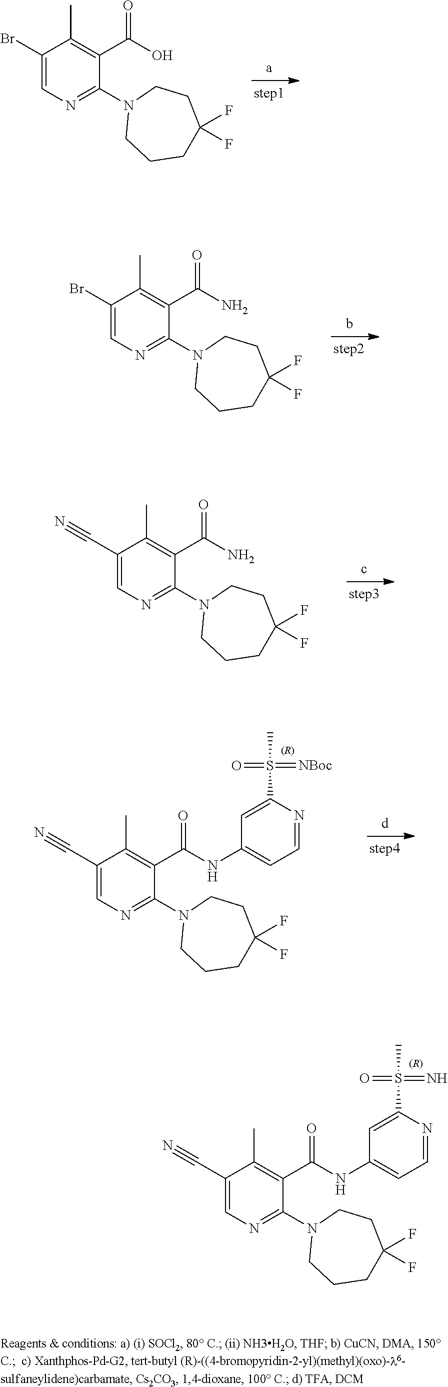

Step 1: 5-bromo-2-(4,4-difluoroazepan-1-yl)-4-methylnicotinamide: A solution of 5-bromo-2-(4,4-difluoroazepan-1-yl)-4-methylnicotinic acid (500 mg, 1.44 mmol) in SOCl₂ (5 mL) was heated to 80° C. and refluxed for 2 hours. The solution was concentrated under vacuum to provide the chloride intermediate. Then the chloride intermediate dissolved in THE (2 mL) was added to a solution of NH₃·H₂O (8 mL) in THE (8 mL) at 0° C. The resulting mixture was stirred at 25° C. for 2 hours. Then the mixture was quenched with water (30 mL) and extracted with DCM (30 mL×3). The combined organic layers were washed with brine, dried over sodium sulfate, concentrated under vacuum. The residue was purified by flash column chromatography on silica gel (PE/EtOAc=1/1) to provide 5-bromo-2-(4,4-difluoroazepan-1-yl)-4-methylnicotinamide (380 mg, 72.5%) as a yellow solid. LCMS (ESI) calcd. for C₁₃H₁₇BrF₂N₃O [M+H]⁺ m/z 348.05, found 347.80.

Step 2: 5-cyano-2-(4,4-difluoroazepan-1-yl)-4-methylnicotinamide: A solution of 5-bromo-2-(4,4-difluoroazepan-1-yl)-4-methylnicotinamide (330 mg, 0.95 mmol) and CuCN (849 mg, 9.48 mmol) in DMA (5 mL) was heated to 150° C. and stirred for 16 hours under nitrogen. After the reaction was completed, the resulting solution was diluted with EtOAc (15 mL) and filtered through celite. The filtrate was washed with water (15 mL×3) and combined organic layers were washed with brine, dried over sodium sulfate, concentrated under vacuum. The residue was purified by flash column chromatography on silica gel (PE/EtOAc=1/1) to provide 5-cyano-2-(4,4-difluoroazepan-1-yl)-4-methylnicotinamide (200 mg, 71.7%) as a yellow solid. LCMS (ESI) calcd. for C₁₄H₁₇F₂N₄O [M+H]⁺ m/z 295.14, found 294.90.

Step 3: tert-butyl (R)-((4-(5-cyano-2-(4,4-difluoroazepan-1-yl)-4-methylnicotinamido)pyridin-2-yl)(methyl)(oxo)-λ⁶-sulfaneylidene)carbamate: A solution of 5-cyano-2-(4,4-difluoroazepan-1-yl)-4-methylnicotinamide (150 mg, 0.51 mmol), tert-butyl (R)-((4-bromopyridin-2-yl)(methyl)(oxo)-λ⁶-sulfaneylidene)carbamate (257 mg, 0.76 mmol), Cs₂CO₃ (498 mg, 1.53 mmol) and Xantphos-Pd-G2 (45 mg, 0.05 mmol) in dioxane (5 mL) was heated to 100° C. and refluxed for 4 hours under nitrogen. After the reaction was completed, the mixture was concentrated. The residue was purified by flash column chromatography on silica gel (PE/EtOAc=1/1) to provide tert-butyl (R)-((4-(5-cyano-2-(4,4-difluoroazepan-1-yl)-4-methylnicotinamido)pyridin-2-yl)(methyl)(oxo)-λ⁶-sulfaneylidene)carbamate (200 mg, 71.3%) as a yellow solid. LCMS (ESI) calcd. for C₂₅H₃₁F₂N₆O₄S [M+H]⁺ m/z 549.21, found 549.10.

Step 4: (R)-5-cyano-2-(4,4-difluoroazepan-1-yl)-4-methyl-N-(2-(S-methylsulfonimidoyl)pyridin-4-yl)nicotinamide: A solution of tert-butyl (R)-((4-(5-cyano-2-(4,4-difluoroazepan-1-yl)-4-methylnicotinamido)pyridin-2-yl)(methyl)(oxo)-λ⁶-sulfaneylidene)carbamate (180 mg, 0.33 mmol) in DCM (5 mL) was added TFA (1 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (15 mL×3). The combined organic layers were washed with brine, dried over sodium sulfate, concentrated under vacuum. The residue was purified by prep-HPLC (Gemini 5 um C₁₈ column, 150*21.2 mm, eluting with 30% to 70% MeCN/H₂O containing 0.05% NH₃) to provide (R)-5-cyano-2-(4,4-difluoroazepan-1-yl)-4-methyl-N-(2-(S-methylsulfonimidoyl)pyridin-4-yl)nicotinamide (73.36 mg, 50%) as a white solid. ¹H NMR (400 MHz, DMSO-d₆) δ 11.30 (s, 1H), 8.63 (d, J=5.4 Hz, 1H), 8.58 (s, 1H), 8.37 (d, J=1.7 Hz, 1H), 7.82 (dd, J=5.4, 1.9 Hz, 1H), 4.40 (s, 1H), 3.72 (s, 2H), 3.57 (t, J=5.6 Hz, 2H), 3.15 (s, 3H), 2.37 (s, 3H), 2.34-2.23 (m, 2H), 2.08-1.94 (m, 2H), 1.89-1.77 (m, 2H). LCMS (ESI) calcd. for C₂₀H₂₃F₂N₆O₂S [M+H]⁺ m/z 449.16, found 449.05.

### Example 202

### (R)-2-(4,4-difluoroazepan-1-yl)-N-(3-(N-(2-(3,3-difluoroazetidin-1-yl)acetyl)-S-methylsulfonimidoyl)phenyl)-4-methyl-5-(trifluoromethyl)nicotinamide

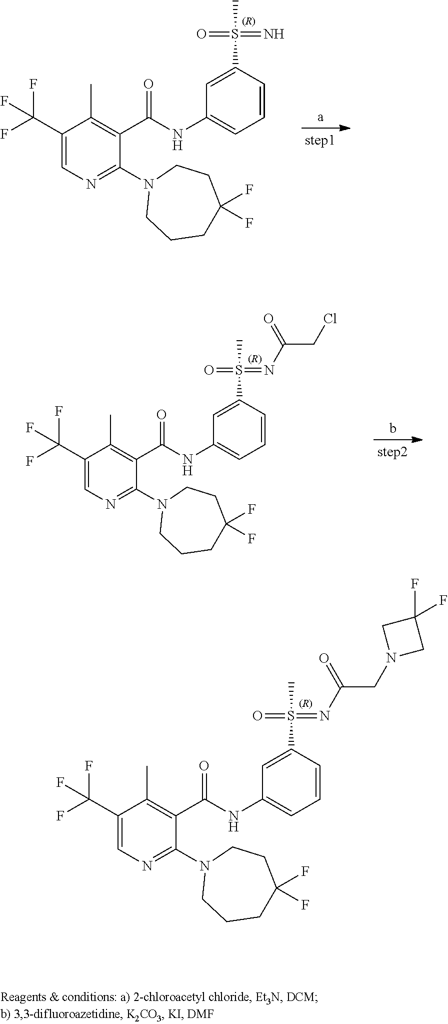

Step 1: (R)-N-(3-(N-(2-chloroacetyl)-S-methylsulfonimidoyl)phenyl)-2-(4,4-difluoroazepan-1-yl)-4-methyl-5-(trifluoromethyl)nicotinamide: A mixture of (R)-2-(4,4-difluoroazepan-1-yl)-4-methyl-N-(3-(S-methylsulfonimidoyl)phenyl)-5-(trifluoromethyl)nicotinamide (110 mg, 0.22 mmol) and Et₃N (68 mg, 0.67 mmol) in DCM (5 mL) was added 2-chloroacetyl chloride (76 mg, 0.67 mmol) at room temperature. The reaction mixture was stirred at room temperature for 16 hours. After the reaction was completed, the resulting solution was diluted with water (15 mL) and extracted with DCM (15 mL×3). The combined organic layers were washed with brine, dried over Na₂SO₄, concentrated under vacuum. The residue was purified by flash column chromatography on silica gel (PE/EtAOc=1/1) to provide (R)-N-(3-(N-(2-chloroacetyl)-S-methylsulfonimidoyl)phenyl)-2-(4,4-difluoroazepan-1-yl)-4-methyl-5-(trifluoromethyl)nicotinamide (100 mg, 77.8%) as a yellow solid. LCMS (ESI) calcd. for C₂₃H₂₅ClF₅N₄O₃S [M+H]⁺ m/z 567.13, found 567.1.

Step 2: (R)-2-(4,4-difluoroazepan-1-yl)-N-(3-(N-(2-(3,3-difluoroazetidin-1-yl)acetyl)-S-methylsulfonimidoyl)phenyl)-4-methyl-5-(trifluoromethyl)nicotinamide: A mixture of (R)-N-(3-(N-(2-chloroacetyl)-S-methylsulfonimidoyl)phenyl)-2-(4,4-difluoroazepan-1-yl)-4-methyl-5-(trifluoromethyl)nicotinamide (90 mg, 0.16 mmol), KI (53 mg, 0.32 mmol) and K₂CO₃ (44 mg, 0.32 mmol) in DMF (5 mL) was added 3,3-difluoroazetidine hydrochloride (41 mg, 0.32 mmol) at room temperature. The reaction mixture was heated to 40° C. and stirred for 16 hours. After the reaction was completed, the resulting solution was diluted with water (15 mL) and extracted with EtOAc (15 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 80% MeCN/H₂O containing 0.05% NH₃) to provide (R)-2-(4,4-difluoroazepan-1-yl)-N-(3-(N-(2-(3,3-difluoroazetidin-1-yl)acetyl)-S-methylsulfonimidoyl)phenyl)-4-methyl-5-(trifluoromethyl)nicotinamide (61.20 mg, 61.1%) as a white solid. ¹H NMR (400 MHz, DMSO-d₆) δ 10.97 (s, 1H), 8.45 (s, 1H), 8.33 (s, 1H), 7.96 (dt, J=6.8, 2.1 Hz, 1H), 7.73-7.63 (m, 2H), 3.75-3.60 (m, 8H), 3.46 (s, 3H), 3.36 (s, 2H), 2.35-2.23 (m, 5H), 2.08-1.96 (m, 2H), 1.88-1.78 (m, 2H). LCMS (ESI) calcd. for C₂₆H₂₉F₇N₅O₃S [M+H]⁺ m/z 624.19, found 624.1.

### Example 203

### (R)-2-(4,4-difluoroazepan-1-yl)-4-methyl-5-(1-methyl-1H-pyrazol-4-yl)-N-(3-(S-methyl-N-(2-(methylamino)ethyl)sulfonimidoyl)phenyl)nicotinamide

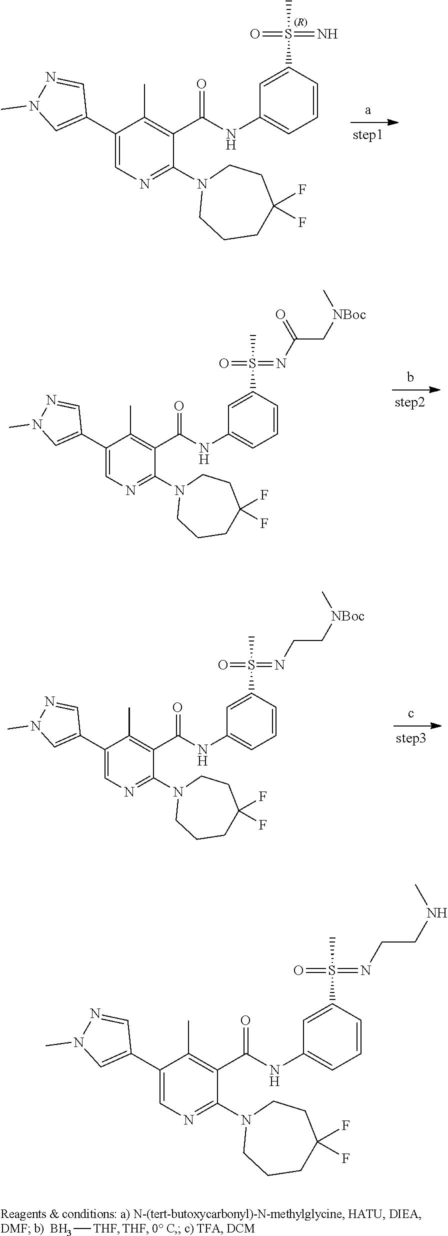

Step 1: tert-butyl (R)-(2-(((3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)amino)-2-oxoethyl)(methyl)carbamate: A solution of (R)-2-(4,4-difluoroazepan-1-yl)-4-methyl-5-(1-methyl-1H-pyrazol-4-yl)-N-(3-(S-methylsulfonimidoyl)phenyl)nicotinamide (300 mg, 0.60 mmol), N-(tert-butoxycarbonyl)-N-methylglycine (226 mg, 1.19 mmol), HATU (340 mg, 0.90 mmol) and DIEA (231 mg, 1.79 mmol) in DMF (10 mL) was stirred at room temperature for 16 hours. LCMS showed the reaction was completed. The mixture was diluted with water (30 mL) and extracted with EtOAc (30 mL×3). The combined organic layers were washed with brine, dried over sodium sulfate, concentrated under vacuum. The residue was purified by flash column chromatography on silica gel (PE/EtOAc=1/1) to provide tert-butyl (R)-(2-(((3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)amino)-2-oxoethyl)(methyl)carbamate (220 mg, 54.7%) as a yellow solid. LCMS (ESI) calcd. for C₃₂H₄₂F₂N₇O₅S [M+H]⁺ m/z 674.30, found 674.20.

Step 2: tert-butyl (R)-(2-(((3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)amino)ethyl)(methyl)carbamate: A solution of tert-butyl (R)-(2-(((3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)amino)-2-oxoethyl)(methyl)carbamate (180 mg, 0.27 mmol) in THE (5 mL) was added BH₃-THF (1 mL) at 0° C. The reaction mixture was stirred at 0° C. for 4 hours. LCMS showed the reaction was completed. The mixture was diluted with water (15 mL) and extracted with EtOAc (15 mL×3). The combined organic layers were washed with brine, dried over sodium sulfate, concentrated under vacuum. The residue was purified by flash column chromatography on silica gel (PE/EtOAc=1/1) to provide tert-butyl (R)-(2-(((3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)amino)ethyl)(methyl)carbamate (50 mg, 28.4%) as a yellow solid. LCMS (ESI) calcd. for C₃₂H₄₄F₂N₇O₄S [M+H]⁺ m/z 660.32, found 660.20.

Step 2: (R)-2-(4,4-difluoroazepan-1-yl)-4-methyl-5-(1-methyl-1H-pyrazol-4-yl)-N-(3-(S-methyl-N-(2-(methylamino)ethyl)sulfonimidoyl)phenyl)nicotinamide: A mixture of tert-butyl (R)-(2-(((3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)amino)ethyl)(methyl)carbamate (50 mg, 0.08 mmol) in DCM (4 mL) was added TFA (0.8 mL) at room temperature. The reaction mixture was stirred at 25° C. for 2 hours. The mixture was diluted with water (10 mL) and extracted with EtOAc (10 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 80% MeCN/H₂O containing 0.05% NH₃) to provide (R)-2-(4,4-difluoroazepan-1-yl)-4-methyl-5-(1-methyl-1H-pyrazol-4-yl)-N-(3-(S-methyl-N-(2-(methylamino)ethyl)sulfonimidoyl)phenyl)nicotinamide (3.20 mg, 7.52%) as a white solid. ¹H NMR (400 MHz, DMSO-d₆) δ 10.86 (s, 1H), 8.30 (s, 1H), 8.18 (s, 1H), 7.91 (d, J=8.5 Hz, 2H), 7.64-7.56 (m, 3H), 3.89 (s, 3H), 3.60-3.52 (m, 4H), 3.13 (s, 3H), 2.90-2.73 (m, 2H), 2.55-2.50 (m, 2H), 2.32-2.20 (m, 8H), 2.04-1.94 (m, 2H), 1.82-1.75 (m, 2H). LCMS (ESI) calcd. for C₂₇H₃₆F₂N₇O₂S [M+H]⁺ m/z 560.26, found 560.2.

### Example 204

### (R)-6-cyano-2-(4,4-difluoroazepan-1-yl)-4-methyl-N-(2-(S-methylsulfonimidoyl)pyridin-4-yl)nicotinamide

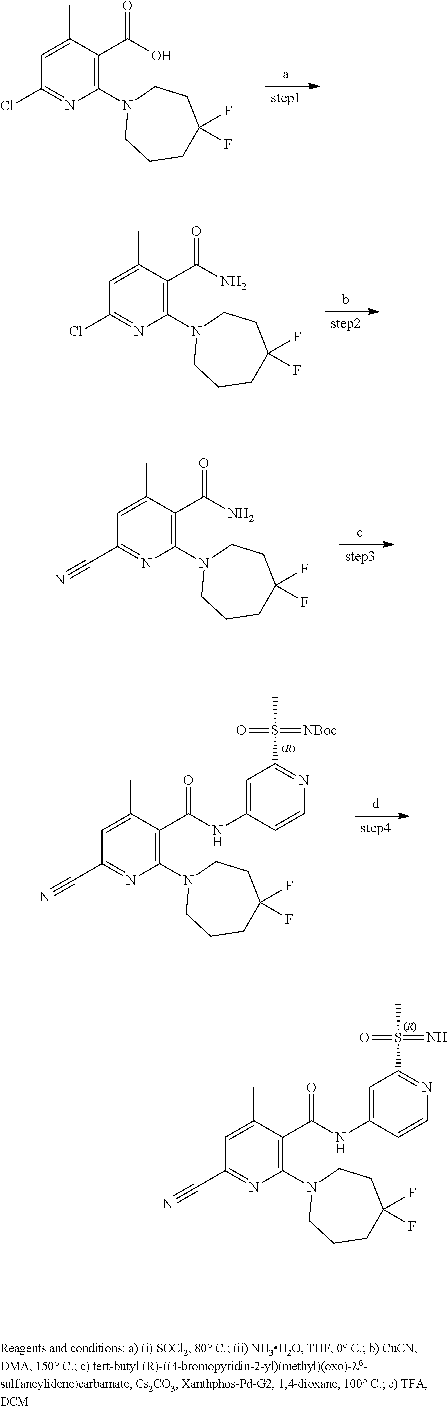

Step 1: 6-chloro-2-(4,4-difluoroazepan-1-yl)-4-methylnicotinamide: A solution of 6-chloro-2-(4,4-difluoroazepan-1-yl)-4-methylnicotinic acid (800 mg, 2.63 mmol) in SOCl₂ (5 mL) was heated to 80° C. and stirred for 2 hours. The solution was concentrated under vacuum to provide the chloride intermediate. Then the chloride intermediate dissolved in THF (2 mL) was added to NH₃·H₂O (6 mL) at 0° C. The resulting mixture was stirred at 25° C. for 2 hours. Then the mixture was quenched with water (30 mL) and extracted with DCM (30 mL×3). The combined organic layers were washed with brine, dried over sodium sulfate, concentrated under vacuum. The residue was purified by flash column chromatography on silica gel (PE/EtOAc=1/1) to provide 6-chloro-2-(4,4-difluoroazepan-1-yl)-4-methylnicotinamide (500 mg, 62.1%) as a yellow solid. LCMS (ESI) calcd. for C₁₃H₁₇ClF₂N₃O [M+H]⁺ m/z 304.10, found 303.9

Step 2: 6-cyano-2-(4,4-difluoroazepan-1-yl)-4-methylnicotinamide: A solution of 6-chloro-2-(4,4-difluoroazepan-1-yl)-4-methylnicotinamide (500 mg, 1.65 mmol) and CuCN (1.47 g, 16.46 mmol) in DMA (20 mL) was heated to 150° C. and stirred for 16 hours under nitrogen. After the reaction was completed, the mixture was cooled to room temperature. The mixture was quenched with water (60 mL) and extracted with DCM (60 mL×3). The combined organic layers were washed with brine, dried over sodium sulfate, concentrated under vacuum. The residue was purified by flash column chromatography on silica gel (PE/EtOAc=1/1) to provide 6-cyano-2-(4,4-difluoroazepan-1-yl)-4-methylnicotinamide (110 mg, 22.5%) as a white solid. LCMS (ESI) calcd. for C₁₄H₁₇F₂N₄O [M+H]⁺ m/z 295.14, found 295.1.

Step 3: tert-butyl (R)-((4-(6-cyano-2-(4,4-difluoroazepan-1-yl)-4-methylnicotinamido)pyridin-2-yl)(methyl)(oxo)-λ⁶-sulfaneylidene)carbamate: A solution of 6-cyano-2-(4,4-difluoroazepan-1-yl)-4-methylnicotinamide (100 mg, 0.34 mmol), tert-butyl (R)-((4-bromopyridin-2-yl)(methyl)(oxo)-16-sulfaneylidene)carbamate (229 mg, 0.68 mmol), Cs₂CO₃ (221 mg, 0.68 mmol) and Xantphos-Pd-G2 (30 mg, 0.03 mmol) in 1,4-dioxane (5 mL) was heated to 100° C. and refluxed for 16 hours under nitrogen. After the reaction was completed, the mixture was cooled to room temperature. The mixture was quenched with water (15 mL) and extracted with DCM (15 mL×3). The combined organic layers were washed with brine, dried over sodium sulfate, concentrated under vacuum. The residue was purified by flash column chromatography on silica gel (PE/EtOAc=1/1) to provide tert-butyl (R)-((4-(6-cyano-2-(4,4-difluoroazepan-1-yl)-4-methylnicotinamido)pyridin-2-yl)(methyl)(oxo)-λ⁶-sulfaneylidene)carbamate (100 mg, 53.5%) as a white solid. LCMS (ESI) calcd. for C₂₅H₃₁F₂N₆O₄S [M+H]⁺ m/z 549.21, found 549.2.

Step 4: (R)-6-cyano-2-(4,4-difluoroazepan-1-yl)-4-methyl-N-(2-(S-methylsulfonimidoyl)pyridin-4-yl)nicotinamide. A solution of tert-butyl (R)-((4-(6-cyano-2-(4,4-difluoroazepan-1-yl)-4-methylnicotinamido)pyridin-2-yl)(methyl)(oxo)-λ⁶-sulfaneylidene)carbamate (100 mg, 0.18 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (15 mL×3). The combined organic layers were washed with brine, dried over sodium sulfate, concentrated under vacuum. The residue was purified by prep-HPLC (Gemini 5 um C₁₈ column, 150*21.2 mm, eluting with 35% to 70% MeCN/H₂O containing 0.05% NH₃) to provide (R)-6-cyano-2-(4,4-difluoroazepan-1-yl)-4-methyl-N-(2-(S-methylsulfonimidoyl)pyridin-4-yl)nicotinamide (70.40 mg, 83.4%) as a white solid. ¹H NMR (400 MHz, DMSO-d₆) δ 11.23 (s, 1H), 8.62 (d, J=5.4 Hz, 1H), 8.36 (s, 1H), 7.81 (d, J=3.8 Hz, 1H), 7.31 (s, 1H), 4.29 (s, 1 H), 3.66-3.48 (m, 4H), 3.15 (s, 3H), 2.34-2.20 (m, 5H), 2.07-1.94 (m, 2H), 1.86-1.77 (m, 2H). LCMS (ESI) calcd. for C₂₀H₂₃F₂N₆O₂S [M+H]⁺ m/z 449.16, found 449.1.

### Example 205 and Example 206

### (S)-5-chloro-2-(4,4-difluoroazepan-1-yl)-N-(4-fluoro-3-(methylsulfinyl)phenyl)-4-(trifluoromethyl)benzamide and (R)-5-chloro-2-(4,4-difluoroazepan-1-yl)-N-(4-fluoro-3-(methylsulfinyl)phenyl)-4-(trifluoromethyl)benzamide

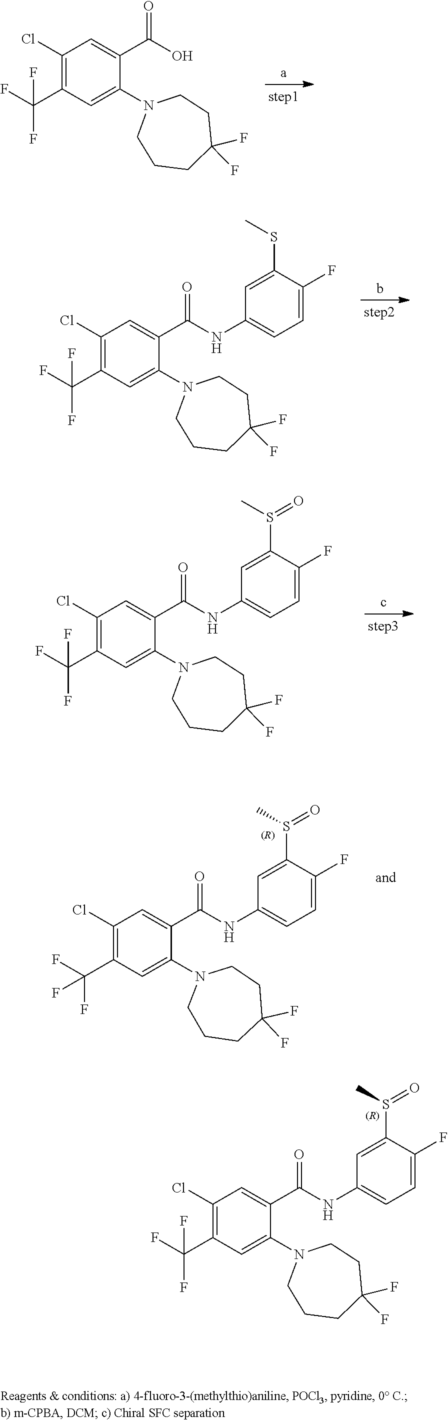

Step 1: 5-chloro-2-(4,4-difluoroazepan-1-yl)-N-(4-fluoro-3-(methylthio)phenyl)-4-(trifluoromethyl)benzamide: A solution of 5-chloro-2-(4,4-difluoroazepan-1-yl)-4-(trifluoromethyl)benzoic acid (400 mg, 1.12 mmol), 4-fluoro-3-(methylthio)aniline (176 mg, 1.12 mmol) in pyridine (5 mL) was added POCl₃ (257 mg, 1.68 mmol) at 0° C. The reaction solution was stirred at room temperature for 2 hours. Then the mixture was quenched with water (50 mL) and extracted with DCM (50 mL×3). The combined organic layers were washed with brine, dried over sodium sulfate, concentrated under vacuum. The residue was purified by flash column chromatography on silica gel (PE/EtOAc=5/1) to provide 5-chloro-2-(4,4-difluoroazepan-1-yl)-N-(4-fluoro-3-(methylthio)phenyl)-4-(trifluoromethyl)benzamide (220 mg, 39.59%) as a yellow oil. LCMS (ESI) calcd. for C₂₁H₂₀ClF₆N₂OS [M+H]⁺ m/z 497.09, found 497.0.

Step 2: 5-chloro-2-(4,4-difluoroazepan-1-yl)-N-(4-fluoro-3-(methylsulfinyl)phenyl)-4-(trifluoromethyl)benzamide: A solution of 5-chloro-2-(4,4-difluoroazepan-1-yl)-N-(4-fluoro-3-(methylthio)phenyl)-4-(trifluoromethyl)benzamide (400 mg, 0.81 mmol) in DCM (10 mL) was added m-CPBA (194 mg, 1.13 mmol) at 0° C. The reaction solution was stirred at room temperature for 1 hour. After the reaction was completed, the resulting solution was diluted with water (50 mL) and extracted with EtOAc (50 mL×3). The combined organic layers were washed with brine, dried over Na₂SO₄, concentrated under vacuum. The residue was purified by flash column chromatography on silica gel (PE/EtOAc=4/1) to give 5-chloro-2-(4,4-difluoroazepan-1-yl)-N-(4-fluoro-3-(methylsulfinyl)phenyl)-4-(trifluoromethyl)benzamide (200 mg, 50.0%) as a white solid. LCMS (ESI) calcd. for C₂₁H₂₀ClF₆N₂O₂S [M+H]⁻ m/z 513.09, found 513.1.

Step 3: (S)-5-chloro-2-(4,4-difluoroazepan-1-yl)-N-(4-fluoro-3-(methylsulfinyl)phenyl)-4-(trifluoromethyl)benzamide & (R)-5-chloro-2-(4,4-difluoroazepan-1-yl)-N-(4-fluoro-3-(methylsulfinyl)phenyl)-4-(trifluoromethyl)benzamide: 5-chloro-2-(4,4-difluoroazepan-1-yl)-N-(4-fluoro-3-(methylsulfinyl)phenyl)-4-(trifluoromethyl)benzamide was further purified by Chiral-Prep-SFC ((R,R)-Whelk-01, 20 mm I.D.*250 mm L, 5 m, eluting with 30% MeOH/CO₂ containing 0.1% NH₃) to give (S)-5-chloro-2-(4,4-difluoroazepan-1-yl)-N-(4-fluoro-3-(methylsulfinyl)phenyl)-4-(trifluoromethyl)benzamide (45.0 mg) as the first elution. ¹H NMR (400 MHz, DMSO-d₆) δ 10.87 (s, 1H), 8.14 (dd, J=6.1, 2.7 Hz, 1H), 7.92-7.84 (m, 1H), 7.71 (s, 1H), 7.40 (t, J=9.1 Hz, 1H), 7.34 (s, 1H), 3.46-3.34 (m, 4H), 2.83 (s, 3H), 2.23 (d, J=6.5 Hz, 2H), 2.13-1.99 (m, 2H), 1.81 (d, J=5.3 Hz, 2H). LCMS (ESI) calcd. for C₂₁H₂₀ClF₆N₂O₂S [M+H]⁺ m/z 513.08, found 513.1 and (R)-5-chloro-2-(4,4-difluoroazepan-1-yl)-N-(4-fluoro-3-(methylsulfinyl)phenyl)-4-(trifluoromethyl)benzamide (42.0 mg) as the second elution. ¹H NMR (400 MHz, DMSO-d₆) δ 10.86 (s, 1H), 8.14 (dd, J=6.0, 2.5 Hz, 1H), 7.92-7.84 (m, 1H), 7.71 (s, 1H), 7.40 (t, J=9.1 Hz, 1H), 7.34 (s, 1H), 3.47-3.34 (m, 4H), 2.83 (s, 3H), 2.22 (dd, J=8.5, 5.5 Hz, 2H), 2.13-1.99 (m, 2H), 1.81 (d, J=5.2 Hz, 2H). LCMS (ESI) calcd. for C₂₁H₂₀ClF₆N₂O₂S [M+H]⁺ m/z 513.08, found 513.1.

### Example 207 and Example 208

### (S)-3-chloro-5-(4,4-difluoroazepan-1-yl)-N-(3-(S-methylsulfonimidoyl)phenyl)-2-(trifluoromethyl)isonicotinamide and (R)-3-chloro-5-(4,4-difluoroazepan-1-yl)-N-(3-(S-methylsulfonimidoyl)phenyl)-2-(trifluoromethyl)isonicotinamide

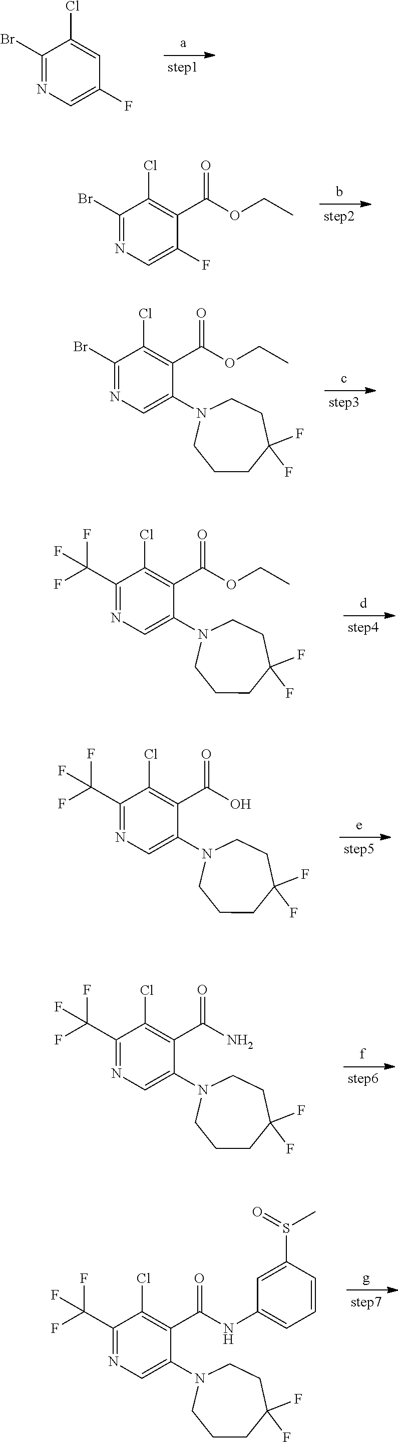

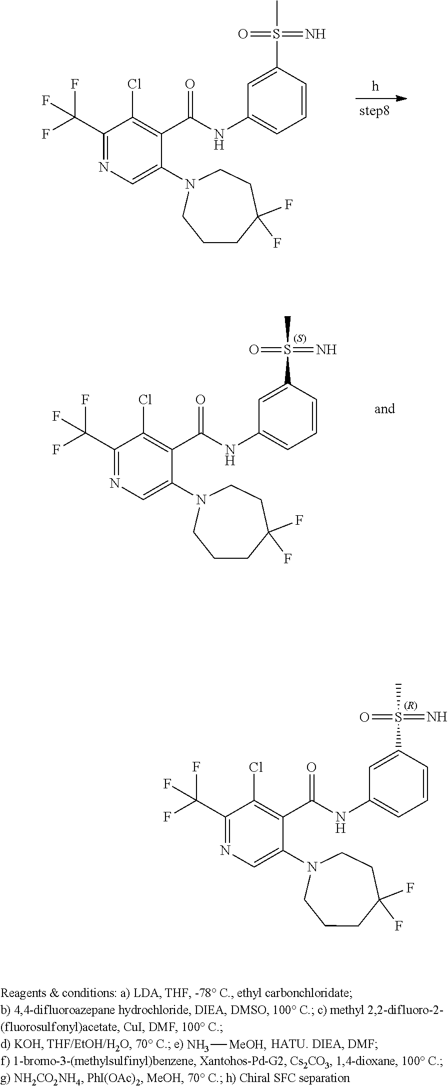

Step 1: ethyl 2-bromo-3-chloro-5-fluoroisonicotinate. To a solution of 2-bromo-3-chloro-5-fluoropyridine (7 g, 0.033 mol) in THF (50 mL) was added LDA (3.88 g, 0.036 mmol) dropwise at −78° C. under N₂ atmosphere. The solution was stirred at the same temperature for 1 hour. Then to the solution was added ethyl carbonochloridate (5.35 g, 0.050 mol) at −78. The resulting solution was slowly warmed to room temperature and stirred for 2 hours. The final mixture was quenched with saturated aqueous NH₄Cl and extracted with EtOAc (50 mL×3). Then the combined organic layers were washed with brine, dried over Na₂SO₄, concentrated under vacuum. The residue was purified by flash column chromatography on silica gel (PE/EtOAc=10/1) to give ethyl 2-bromo-3-chloro-5-fluoroisonicotinate (5 g, 53.94%) as a yellow oil. LCMS (ESI) calcd. for ClH₇BrClFNO₂ [M+H]t m/z 281.93, found 281.90.

Step 2: ethyl 2-bromo-3-chloro-5-(4,4-difluoroazepan-1-yl)isonicotinate: A solution of ethyl 2-bromo-3-chloro-5-fluoroisonicotinate (5 g, 0.018 mol) and DIEA (6.85 g, 0.53 mol) in DMSO (20 ml) was added 4,4-difluoroazepane hydrochloride (6.07 g, 0.035 mol). Then the mixture was heated at 100° C. for 10 hours. After the reaction was completed, the resulting solution was diluted with water (60 mL) and extracted with EtOAc (50 mL×3). The combined organic layers were washed with brine, dried over Na₂SO₄, concentrated under vacuum. The residue was purified by flash column chromatography on silica gel (PE/EtOAc=10/1) to give ethyl 2-bromo-3-chloro-5-(4,4-difluoroazepan-1-yl)isonicotinate (1.2 g, 16.8%) as a yellow solid. LCMS (ESI) calcd. for C₁₄H₁₇BrClF₂N₂O₂ [M+H]⁺ m/z 399.01, found 398.80.

Step 3: ethyl 3-chloro-5-(4,4-difluoroazepan-1-yl)-2-(trifluoromethyl)isonicotinate: A solution of ethyl 2-bromo-3-chloro-5-(4,4-difluoroazepan-1-yl)isonicotinate (1.2 g, 0.003 mol) and CuI (1.15 g, 0.006 mol) in DMF (10 ml) was added methyl 2,2-difluoro-2-(fluorosulfonyl)acetate (2.90 g, 0.015 mol) at 100° C. and stirred for 10 hours under N₂. After the reaction was completed, the resulting solution was diluted with water (50 mL) and extracted with EtOAc (30 mL×3). The combined organic layers were washed with brine, dried over Na₂SO₄, concentrated under vacuum. The residue was purified by flash column chromatography on silica gel (PE/EtOAc=10/1) to give ethyl 3-chloro-5-(4,4-difluoroazepan-1-yl)-2-(trifluoromethyl)isonicotinate (0.9 g, 77.6%) as a yellow solid. LCMS (ESI) calcd. for C₁₅H₁₇ClF₅N₂O₂ [M+H]⁺ m/z 387.09, found 387.00.

Step 4: 3-chloro-5-(4,4-difluoroazepan-1-yl)-2-(trifluoromethyl)isonicotinic acid: A solution of ethyl 3-chloro-5-(4,4-difluoroazepan-1-yl)-2-(trifluoromethyl)isonicotinate (900 mg, 2.33 mmol) in THF/H₂O/EtOH=1:1:1 (9 ml) was added KOH (1301 mg, 23.30 mmol). The mixture was heated at 70° C. for 16 hours. After the reaction was completed, the organic solvent was removed under reduced pressure. The aqueous solution was adjusted to pH 2-3 with 2N HCl (10 mL). Then the solution was extracted with EtOAc (20 mL×3). The combined organic layers were washed with brine, dried over Na₂SO₄, concentrated under vacuum. The residue was purified by flash column chromatography on silica gel (DCM/MeOH=10/1) to give 3-chloro-5-(4,4-difluoroazepan-1-yl)-2-(trifluoromethyl)isonicotinic acid (410 mg, 49.2%) as a yellow solid. LCMS (ESI) calcd. for C₁₃H₁₃ClF₅N₂O₂ [M+H]⁺ m/z 359.06, found 358.70.

Step 5: 3-chloro-5-(4,4-difluoroazepan-1-yl)-2-(trifluoromethyl)isonicotinamide: A solution of 3-chloro-5-(4,4-difluoroazepan-1-yl)-2-(trifluoromethyl)isonicotinic acid (410 mg, 1.15 mmol), HATU (869 mg, 2.29 mmol) and DIEA (739 mg, 5.72 mmol) in DMF (5 ml) was added NH₃-MeOH (7M, 1 mL) at 25° C. The mixture was stirred at the same temperature for 16 hours. After the reaction was completed, the resulting solution was diluted with water (20 mL) and extracted with EtOAc (20 mL×3). The combined organic layers were washed with brine, dried over Na₂SO₄, concentrated under vacuum. The residue was purified by flash column chromatography on silica gel (PE/EtOAc=1/1) to give 3-chloro-5-(4,4-difluoroazepan-1-yl)-2-(trifluoromethyl)isonicotinamide (200 mg, 44.0%) as a yellow solid. LCMS (ESI) calcd. for C₁₃H₁₄ClF₅N₃O [M+H]⁺ m/z 358.07, found 357.90.

Step 6: 3-chloro-5-(4,4-difluoroazepan-1-yl)-N-(3-(methylsulfinyl)phenyl)-2-(trifluoromethyl)isonicotinamide: A mixture of 3-chloro-5-(4,4-difluoroazepan-1-yl)-2-(trifluoromethyl)isonicotinamide (200 mg, 0.56 mmol), Cs₂CO₃ (547 mg, 1.68 mmol) and Xantohos-Pd-G2 (99 mg, 0.11 mmol) in 1,4-dioxane (5 mL) was added 1-bromo-3-(methylsulfinyl)benzene (306 mg, 1.40 mmol). The mixture was heated at 100° C. for 16 hours under N₂. After the reaction was completed, the resulting solution was diluted with water (20 mL) and extracted with EtOAc (20 mL×3). The combined organic layers were washed with brine, dried over Na₂SO₄, concentrated under vacuum. The residue was purified by flash column chromatography on silica gel (PE/EtOAc=1/5 to 1/1) to give 3-chloro-5-(4,4-difluoroazepan-1-yl)-N-(3-(methylsulfinyl)phenyl)-2-(trifluoromethyl)isonicotinamide (130 mg, 42.2%) as a yellow solid. LCMS (ESI) calcd. for C₂₀H₂₀ClF₅N₃O₂S [M+H]⁺ m/z 496.09, found 495.80.

Step 7: 3-chloro-5-(4,4-difluoroazepan-1-yl)-N-(3-(S-methylsulfonimidoyl)phenyl)-2-(trifluoromethyl)isonicotinamide: A solution of 3-chloro-5-(4,4-difluoroazepan-1-yl)-N-(3-(methylsulfinyl)phenyl)-2-(trifluoromethyl)isonicotinamide (130 mg, 0.26 mmol), PhI(OAc)₂ (211 mg, 0.66 mmol) in MeOH (3 ml) was added ammonium carbamate (61 mg, 0.79 mmol). Then the solution was heated at 70° C. for 16 hours. After the reaction was completed, the resulting solution was diluted with water (50 mL) and extracted with EtOAc (10 mL×3). The combined organic layers were washed with brine, dried over Na₂SO₄, concentrated under vacuum. Then the residue was purified by prep-HPLC (Gemini 5 um C₁₈ column, 150*21.2 mm, eluting with 40% to 95% MeCN/H₂O containing 0.05% NH₃-H₂O) to obtain 3-chloro-5-(4,4-difluoroazepan-1-yl)-N-(3-(S-methylsulfonimidoyl)phenyl)-2-(trifluoromethyl)isonicotinamide (44 mg, 32.8%) as a white solid. LCMS (ESI) calcd. for C₂₀H₂₁ClF₅N₄O₂S [M+H]⁺ m/z 511.10, found 511.00.

Step 8: (S)-3-chloro-5-(4,4-difluoroazepan-1-yl)-N-(3-(S-methylsulfonimidoyl)phenyl)-2-(trifluoromethyl)isonicotinamide and (R)-3-chloro-5-(4,4-difluoroazepan-1-yl)-N-(3-(S-methylsulfonimidoyl)phenyl)-2-(trifluoromethyl)isonicotinamide: 3-chloro-5-(4,4-difluoroazepan-1-yl)-N-(3-(S-methylsulfonimidoyl)phenyl)-2-(trifluoromethyl)isonicotinamide was further purified by Chiral-Prep-SFC (DAICEL 1H, 20 mm I.D.*250 mm L, 5 m, eluting with 30% MeOH/CO₂ containing 0.1% NH₃) to provide (S)-3-chloro-5-(4,4-difluoroazepan-1-yl)-N-(3-(S-methylsulfonimidoyl)phenyl)-2-(trifluoromethyl)isonicotinamide (15.2 mg) as the first elution. ¹H NMR (400 MHz, DMSO-d₆) δ 11.20 (s, 1H), 8.38 (s, 1H), 8.27 (s, 1H), 7.88 (d, J=8.3 Hz, 1H), 7.72 (d, J=7.9 Hz, 1H), 7.62 (t, J=7.9 Hz, 1H), 4.26 (s, 1H), 3.68-3.54 (m, 4H), 3.07 (s, 3H), 2.32-2.20 (m, 2H), 2.15-2.00 (m, 2H), 1.90-1.80 (m, 2H). LCMS (ESI) calcd. for C₂₀H₂₁ClF₅N₄O₂S [M+H]⁺ m/z 511.10, found 511.00 and (R)-3-chloro-5-(4,4-difluoroazepan-1-yl)-N-(3-(S-methylsulfonimidoyl)phenyl)-2-(trifluoromethyl)isonicotinamide (19.4 mg) as the second elution. ¹H NMR (400 MHz, DMSO-d₆) δ 11.20 (s, 1H), 8.38 (s, 1H), 8.27 (s, 1H), 7.88 (d, J=8.2 Hz, 1H), 7.72 (d, J=7.9 Hz, 1H), 7.62 (t, J=7.9 Hz, 1H), 4.26 (s, 1H), 3.66-3.52 (m, 4H), 3.07 (s, 3H), 2.33-2.20 (m, 2H), 2.15-2.00 (m, 2H), 1.90-1.80 (m, 2H). LCMS (ESI) calcd. for C₂₀H₂₁ClF₅N₄O₂S [M+H]⁺ m/z 511.10, found 511.00.

### Example 209

### (R)-N-(3-(N-(2-aminoethyl)-S-methylsulfonimidoyl)phenyl)-5-bromo-2-(4,4-difluoroazepan-1-yl)-4-methylnicotinamide 2,2,2-trifluoroacetate

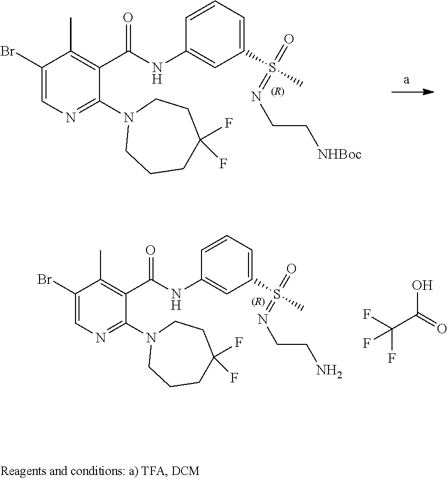

A solution of tert-butyl (R)-(2-(((3-(5-bromo-2-(4,4-difluoroazepan-1-yl)-4-methylnicotinamido)phenyl)(methyl)(oxo)-λ⁶-sulfaneylidene)amino)ethyl)carbamate (60 mg, 0.09 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (10 mL×3). The combined organic layers were washed with brine, dried over sodium sulfate, concentrated under vacuum. The residue was purified by prep-HPLC (Gemini 5 um C₁₈ column, 150*21.2 mm, eluting with 30% to 70% ACN/H₂O containing 0.1% TFA) to provide (R)-N-(3-(N-(2-aminoethyl)-S-methylsulfonimidoyl)phenyl)-5-bromo-2-(4,4-difluoroazepan-1-yl)-4-methylnicotinamide 2,2,2-trifluoroacetate (31.2 mg, 61.5%) as a white solid. ¹H NMR (400 MHz, DMSO-d₆) δ 10.94 (s, 1H), 8.32 (s, 2H), 7.92-7.85 (m, 1H), 7.80-7.68 (m, 3H), 7.67-7.62 (m, 2H), 3.66-3.48 (m, 4H), 3.21 (s, 3H), 3.08-2.83 (m, 4H), 2.35-2.17 (m, 5H), 2.09-1.91 (m, 2H), 1.86-1.72 (m, 2H). LCMS (ESI) calcd. for C₂₂H₂₉BrF₂N₅O₂S [M+H]⁺ m/z 544.12, found 546.1.

### Example 210

### (R)-N-(3-(N-(2-aminoethyl)-S-methylsulfonimidoyl)phenyl)-2-(4,4-difluoroazepan-1-yl)-4-methyl-5-(1-methyl-1H-pyrazol-4-yl)nicotinamide 2,2,2-trifluoroacetate

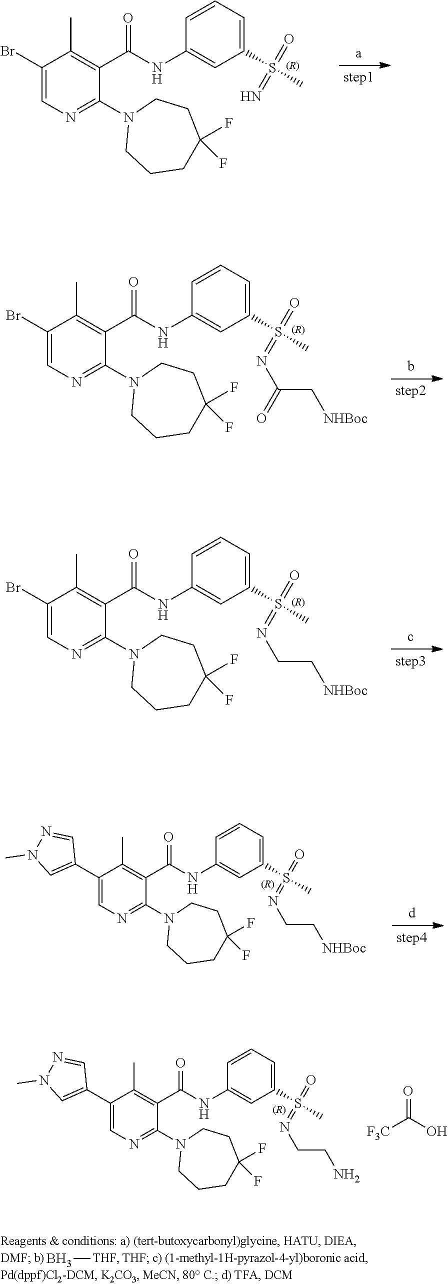

Step 1: tert-butyl (R)-(2-(((3-(5-bromo-2-(4,4-difluoroazepan-1-yl)-4-methylnicotinamido)phenyl)(methyl)(oxo)-λ⁶-sulfaneylidene)amino)-2-oxoethyl)carbamate. A solution of (R)-5-bromo-2-(4,4-difluoroazepan-1-yl)-4-methyl-N-(3-(S-methylsulfonimidoyl)phenyl)nicotinamide (400 mg, 0.80 mmol), (tert-butoxycarbonyl)glycine (280 mg, 1.60 mmol), HATU (455 mg, 1.20 mmol) and DIEA (309 mg, 2.39 mmol) in DMF (8 mL) was stirred at room temperature for 16 hours. LCMS showed the reaction was completed. The mixture was diluted with water (20 mL) and extracted with EtOAc (20 mL×3). The combined organic layers were washed with brine, dried over sodium sulfate, concentrated under vacuum. The residue was purified by flash column chromatography on silica gel (PE/EtOAc=1/1) to provide tert-butyl (R)-(2-(((3-(5-bromo-2-(4,4-difluoroazepan-1-yl)-4-methylnicotinamido)phenyl)(methyl)(oxo)-λ⁶-sulfaneylidene)amino)-2-oxoethyl)carbamate (450 mg, 85.5%) as a yellow solid. LCMS (ESI) calcd. for C₂₇H₃₅BrF₂N₅O₅S [M+H]⁺ m/z 658.15, found 660.0.

Step 2: tert-butyl (R)-(2-(((3-(5-bromo-2-(4,4-difluoroazepan-1-yl)-4-methylnicotinamido)phenyl)(methyl)(oxo)-λ⁶-sulfaneylidene)amino)ethyl)carbamate: A solution of tert-butyl (R)-(2-(((3-(5-bromo-2-(4,4-difluoroazepan-1-yl)-4-methylnicotinamido)phenyl)(methyl)(oxo)-λ⁶-sulfaneylidene)amino)-2-oxoethyl)carbamate (450 mg, 0.68 mmol) in THE (6 mL) was added BH₃-THF (1.2 mL) at 0° C. The reaction mixture was stirred at 0° C. for 3 hours. LCMS showed the reaction was completed. The mixture was quenched with water (20 mL) and extracted with EtOAc (20 mL×3). The combined organic layers were washed with brine, dried over sodium sulfate, concentrated under vacuum. The residue was purified by flash column chromatography on silica gel (PE/EtOAc=1/1) to provide tert-butyl (R)-(2-(((3-(5-bromo-2-(4,4-difluoroazepan-1-yl)-4-methylnicotinamido)phenyl)(methyl)(oxo)-16-sulfaneylidene)amino)ethyl)carbamate (340 mg, 77.2%) as a yellow solid. LCMS (ESI) calcd. for C₂₇H₃₇BrF₂N₅O₄S [M+H]⁺ m/z 644.17, found 644.1.

Step 3: tert-butyl (R)-(2-(((3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)amino)ethyl)carbamate: A solution of tert-butyl (R)-(2-(((3-(5-bromo-2-(4,4-difluoroazepan-1-yl)-4-methylnicotinamido)phenyl)(methyl)(oxo)-λ⁶-sulfaneylidene)amino)ethyl)carbamate (250 mg, 0.39 mmol), (1-methyl-1H-pyrazol-4-yl)boronic acid (98 mg, 0.78 mmol), K₂CO₃ (161 mg, 1.16 mmol) and Pd(dppf)Cl₂-DCM (32 mg, 0.04 mmol) in MeCN (6 mL) was heated to 80° C. and refluxed for 16 hours. After the reaction was completed, the mixture was concentrated. The residue was purified by flash column chromatography on silica gel (PE/EtOAc=1/1) to provide tert-butyl (R)-(2-(((3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)amino)ethyl)carbamate (200 mg, 79.8%) as a yellow solid. LCMS (ESI) calcd. for C₃₁H₄₂F₂N₇O₄S [M+H]⁺ m/z 646.30, found 646.2.

Step 4: (R)-N-(3-(N-(2-aminoethyl)-S-methylsulfonimidoyl)phenyl)-2-(4,4-difluoroazepan-1-yl)-4-methyl-5-(1-methyl-1H-pyrazol-4-yl)nicotinamide 2,2,2-trifluoroacetate: A solution of tert-butyl (R)-(2-(((3-(2-(4,4-difluoroazepan-1-yl)-4-methyl-5-(1-methyl-1H-pyrazol-4-yl)nicotinamido)phenyl)(methyl)(oxo)-λ⁶-sulfaneylidene)amino)ethyl)carbamate (200 mg, 0.31 mmol) in DCM (6 mL) was added TFA (1 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (15 mL×3). The combined organic layers were washed with brine, dried over sodium sulfate, concentrated under vacuum. The residue was purified by prep-HPLC (Gemini 5 um C₁₈ column, 150*21.2 mm, eluting with 20% to 60% ACN/H₂O containing 0.1% TFA) to provide (R)-N-(3-(N-(2-aminoethyl)-S-methylsulfonimidoyl)phenyl)-2-(4,4-difluoroazepan-1-yl)-4-methyl-5-(1-methyl-1H-pyrazol-4-yl)nicotinamide 2,2,2-trifluoroacetate (115.60 mg, 68.4%) as a white solid. ¹H NMR (400 MHz, DMSO-d₆) δ 10.90 (s, 1H), 8.36 (s, 1H), 8.18 (s, 1H), 7.93-7.87 (m, 2H), 7.78-7.66 (m, 3H), 7.65-7.60 (m, 3H), 3.89 (s, 3H), 3.64-3.50 (m, 4H), 3.21 (s, 3H), 3.10-2.82 (m, 4H), 2.33-2.21 (m, 5H), 2.08-1.95 (m, 2H), 1.85-1.74 (m, 2H). LCMS (ESI) calcd. for C₂₆H₃₄F₂N₇O₂S [M+H]⁺ m/z 546.25, found 546.2.

### Example 211

### (R)-5-bromo-2-(4,4-difluoroazepan-1-yl)-4-methyl-N-(3-(S-methyl-N-(2-(methylamino)ethyl)sulfonimidoyl)phenyl)nicotinamide 2,2,2-trifluoroacetate

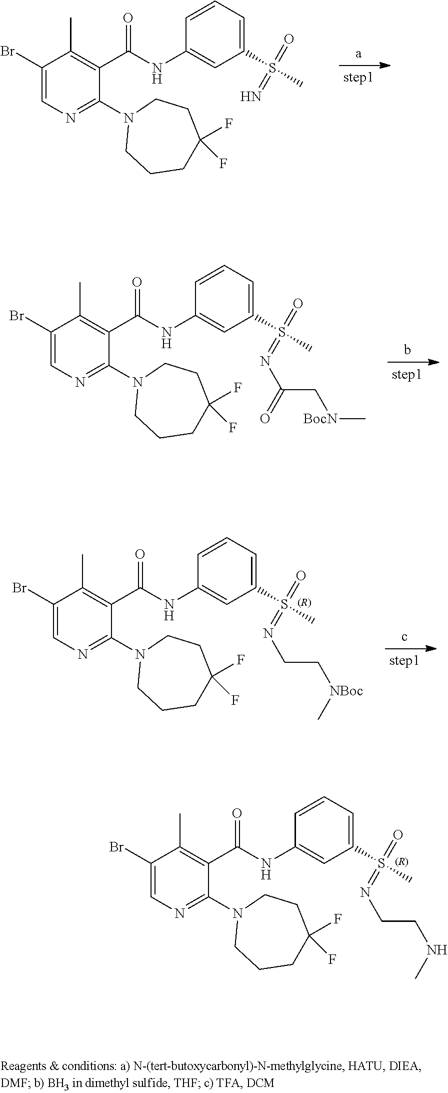

Step 1: tert-butyl (R)-(2-(((3-(5-bromo-2-(4,4-difluoroazepan-1-yl)-4-methylnicotinamido)phenyl)(methyl)(oxo)-λ⁶-sulfaneylidene)amino)-2-oxoethyl)(methyl)carbamate: A solution of (R)-5-bromo-2-(4,4-difluoroazepan-1-yl)-4-methyl-N-(3-(S-methylsulfonimidoyl)phenyl)nicotinamide (400 mg, 0.80 mmol), N-(tert-butoxycarbonyl)-N-methylglycine (302 mg, 1.60 mmol), HATU (455 mg, 1.20 mmol) and DIEA (309 mg, 2.39 mmol) in DMF (8 mL) was stirred at room temperature for 16 hours. LCMS showed the reaction was completed. The mixture was diluted with water (30 mL) and extracted with EtOAc (30 mL×3). The combined organic layers were washed with brine, dried over sodium sulfate, concentrated under vacuum. The residue was purified by flash column chromatography on silica gel (PE/EtOAc=1/1) to provide tert-butyl (R)-(2-(((3-(5-bromo-2-(4,4-difluoroazepan-1-yl)-4-methylnicotinamido)phenyl)(methyl)(oxo)-λ⁶-sulfaneylidene)amino)-2-oxoethyl)(methyl)carbamate (460 mg, 85.6%) as a yellow solid. LCMS (ESI) calcd. for C₂₈H₃₇BrF₂N₅O₅S [M+H]⁺ m/z 672.17, found 672.00.

Step 2: tert-butyl (R)-(2-(((3-(5-bromo-2-(4,4-difluoroazepan-1-yl)-4-methylnicotinamido)phenyl)(methyl)(oxo)-λ⁶-sulfaneylidene)amino)ethyl)(methyl)carbamate: A solution of tert-butyl (R)-(2-(((3-(5-bromo-2-(4,4-difluoroazepan-1-yl)-4-methylnicotinamido)phenyl)(methyl)(oxo)-λ⁶-sulfaneylidene)amino)-2-oxoethyl)(methyl)carbamate (460 mg, 0.68 mmol) in THE (6 mL) was added BH₃ in Dimethyl sulfide (1.2 mL) at 0° C. The reaction mixture was stirred at 0° C. for 6 hours. LCMS showed the reaction was completed. The mixture was diluted with water (30 mL) and extracted with EtOAc (30 mL×3). The combined organic layers were washed with brine, dried over sodium sulfate, concentrated under vacuum. The residue was purified by flash column chromatography on silica gel (PE/EtOAc=1/1) to provide tert-butyl (R)-(2-(((3-(5-bromo-2-(4,4-difluoroazepan-1-yl)-4-methylnicotinamido)phenyl)(methyl)(oxo)-λ⁶-sulfaneylidene)amino)ethyl)(methyl)carbamate (300 mg, 66.6%) as a yellow solid. LCMS (ESI) calcd. for C₂₈H₃₉BrF₂N₅O₄S [M+H]⁺ m/z 658.19, found 660.1.

Step 3: provide (R)-5-bromo-2-(4,4-difluoroazepan-1-yl)-4-methyl-N-(3-(S-methyl-N-(2-(methylamino)ethyl)sulfonimidoyl)phenyl)nicotinamide 2,2,2-trifluoroacetate: A solution of tert-butyl (R)-(2-(((3-(5-bromo-2-(4,4-difluoroazepan-1-yl)-4-methylnicotinamido)phenyl)(methyl)(oxo)-λ⁶-sulfaneylidene)amino)ethyl)(methyl)carbamate (60 mg, 0.09 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (10 mL×3). The combined organic layers were washed with brine, dried over sodium sulfate, concentrated under vacuum. The residue was purified by prep-HPLC (Gemini 5 um C₁₈ column, 150*21.2 mm, eluting with 30% to 70% ACN/H₂O containing 0.1% TFA) to provide (R)-5-bromo-2-(4,4-difluoroazepan-1-yl)-4-methyl-N-(3-(S-methyl-N-(2-(methylamino)ethyl)sulfonimidoyl)phenyl)nicotinamide 2,2,2-trifluoroacetate (31.5 mg, 61.9%) as a white solid. ¹H NMR (400 MHz, DMSO-d₆) δ 10.95 (s, 1H), 8.33 (d, J=4.8 Hz, 4H), 7.88 (dt, J=6.9, 2.2 Hz, 1H), 7.69-7.62 (m, 2H), 3.67-3.45 (m, 4H), 3.22 (s, 3H), 3.13-2.89 (m, 4H), 2.58 (t, J=5.5 Hz, 3H), 2.34-2.19 (m, 5H), 2.05-1.92 (m, 2H), 1.84-1.73 (m, 2H). LCMS (ESI) calcd. for C₂₃H₃₁BrF₂N₅O₂S [M+H]⁺ m/z 558.13, found 560.10.

### Example 212

### (R)-5-chloro-2-(4,4-difluoroazepan-1-yl)-4-methyl-6-(1-methyl-1H-pyrazol-4-yl)-N-(2-(S-methylsulfonimidoyl)pyridin-4-yl)nicotinamide

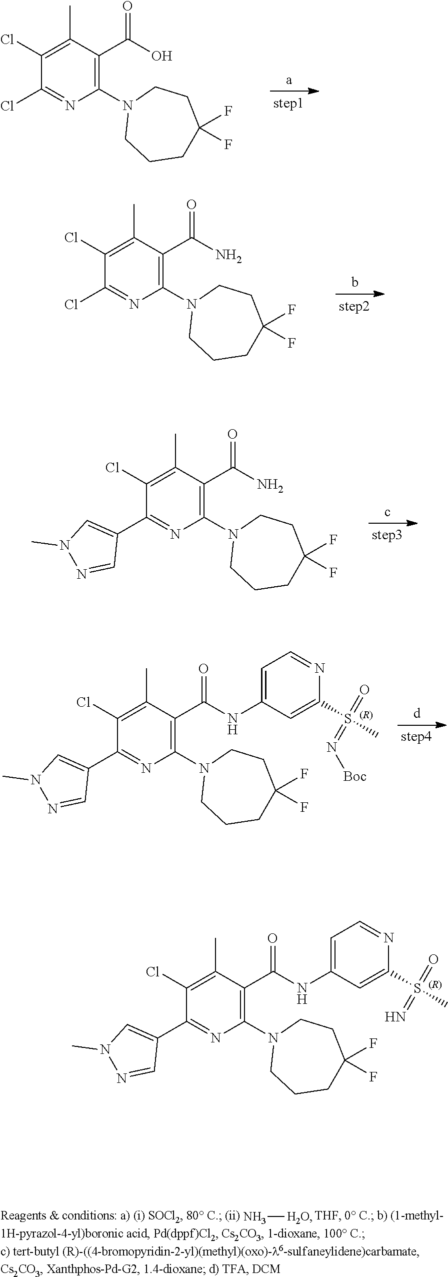

Step 1: 5,6-dichloro-2-(4,4-difluoroazepan-1-yl)-4-methylnicotinamide: A solution of 5,6-dichloro-2-(4,4-difluoroazepan-1-yl)-4-methylnicotinic acid (300 mg, 0.88 mmol) in SOCl₂ (5 mL) was heated to 80° C. and stirred for 2 hours. The solution was concentrated under vacuum to provide the chloride intermediate. Then the chloride intermediate was added to a stirred solution of NH₃·H₂O (5 mL) in THE (8 mL) at 0° C. The resulting mixture was stirred at 25° C. for 0.5 hour. Then the mixture was quenched with water (20 mL) and extracted with EtOAc (20 mL×3). The combined organic layers were washed with brine, dried over sodium sulfate, concentrated under vacuum to provide 5,6-dichloro-2-(4,4-difluoroazepan-1-yl)-4-methylnicotinamide (260 mg, 86.96%) as a yellow solid. LCMS (ESI) calcd. for C₁₃H₁₆C₁₂F₂N₃O [M+H]⁺ m/z 338.07, found 338.0.

Step 2: 5-chloro-2-(4,4-difluoroazepan-1-yl)-4-methyl-6-(1-methyl-1H-pyrazol-4-yl)nicotinamide: A solution of 5,6-dichloro-2-(4,4-difluoroazepan-1-yl)-4-methylnicotinamide (260 mg, 0.77 mmol), (1-methyl-1H-pyrazol-4-yl)boronic acid (194 mg, 1.54 mmol), Cs₂CO₃ (751 mg, 2.31 mmol) and Pd(dppf)Cl₂ (113 mg, 0.15 mmol) in 1,4-dioxane (10 mL) and H₂O (2.5 mL) was heated to 100° C. and refluxed for 16 hours. LCMS showed the reaction was completed. The mixture was diluted with water (20 mL) and extracted with EtOAc (20 mL×3). The combined organic layers were washed with brine, dried over sodium sulfate, concentrated under vacuum. The residue was purified by flash column chromatography on silica gel (PE/EtOAc=1/1) to provide 5-chloro-2-(4,4-difluoroazepan-1-yl)-4-methyl-6-(1-methyl-1H-pyrazol-4-yl)nicotinamide (170 mg, 57.63%) as a yellow solid. LCMS (ESI) calcd. for C₁₇H₂₁ClF₂N₅O [M+H]⁺ m/z 384.14, found 384.0.

Step 3: tert-butyl (R)-((4-(5-chloro-2-(4,4-difluoroazepan-1-yl)-4-methyl-6-(1-methyl-1H-pyrazol-4-yl)nicotinamido)pyridin-2-yl)(methyl)(oxo)-λ⁶-sulfaneylidene)carbamate: A solution of 5-chloro-2-(4,4-difluoroazepan-1-yl)-4-methyl-6-(1-methyl-1H-pyrazol-4-yl)nicotinamide (170 mg, 0.44 mmol), tert-butyl (R)-((4-bromopyridin-2-yl)(methyl)(oxo)-λ⁶-sulfaneylidene)carbamate (164 mg, 0.49 mmol), Cs₂CO₃ (433 mg, 1.33 mmol) and Xantphos-Pd-G2 (79 mg, 0.09 mmol) in 1,4-dioxane (8 mL) was heated to 100° C. and refluxed for 16 hours. LCMS showed the reaction was completed. The mixture was diluted with water (20 mL) and extracted with EtOAc (20 mL×3). The combined organic layers were washed with brine, dried over sodium sulfate, concentrated under vacuum. The residue was purified by flash column chromatography on silica gel (PE/EtOAc=1/1) to provide tert-butyl (R)-((4-(5-chloro-2-(4,4-difluoroazepan-1-yl)-4-methyl-6-(1-methyl-1H-pyrazol-4-yl)nicotinamido)pyridin-2-yl)(methyl)(oxo)-λ⁶-sulfaneylidene)carbamate (200 mg, 70.92%) as yellow oil. LCMS (ESI) calcd. for C₂₈H₃₅ClF₂N₇O₄S [M+H]⁺ m/z 638.21, found 638.2.

Step 4: (R)-5-chloro-2-(4,4-difluoroazepan-1-yl)-4-methyl-6-(1-methyl-1H-pyrazol-4-yl)-N-(2-(S-methylsulfonimidoyl)pyridin-4-yl)nicotinamide: A solution of tert-butyl (R)-((4-(5-chloro-2-(4,4-difluoroazepan-1-yl)-4-methyl-6-(1-methyl-1H-pyrazol-4-yl)nicotinamido)pyridin-2-yl)(methyl)(oxo)-λ⁶-sulfaneylidene)carbamate (200 mg, 0.31 mmol) in DCM (5 mL) was added TFA (0.5 mL) at room temperature. The reaction mixture was stirred at room temperature for 1 hour. After the reaction was completed, the mixture was concentrated. The residue was adjusted to pH=8-9 with saturated aqueous NaHCO₃. Then the aqueous solution was extracted with DCM (10 mL×3). The combined organic layers were washed with brine, dried over sodium sulfate, concentrated under vacuum. The residue was purified by prep-HPLC (Gemini 5 um C₁₈ column, 150*21.2 mm, eluting with 20% to 95% MeCN/H₂O containing 0.05% NH₃·H₂O) to provide (R)-5-chloro-2-(4,4-difluoroazepan-1-yl)-4-methyl-6-(1-methyl-1H-pyrazol-4-yl)-N-(2-(S-methylsulfonimidoyl)pyridin-4-yl)nicotinamide (126.50 mg, 75.03%) as a white solid. ¹H NMR (400 MHz, DMSO-d₆) δ 11.26 (s, 1H), 8.62 (d, J=5.4 Hz, 1H), 8.46 (s, 1H), 8.40 (d, J=1.8 Hz, 1H), 8.11 (s, 1H), 7.82 (dd, J=5.4, 2.0 Hz, 1H), 4.38 (s, 1H), 3.93 (s, 3H), 3.70-3.62 (m, 2H), 3.52 (t, J=6.0 Hz, 2H), 3.15 (s, 3H), 2.32 (s, 5H), 2.06-1.94 (m, 2H), 1.86-1.76 (m, 2H). LCMS (ESI) calcd. for C₂₃H₂₇ClF₂N₇O₂S [M+H]⁺ m/z 538.16, found 538.1.

### Example 213 and Example 214

### (S)-2-(4,4-difluoroazepan-1-yl)-N-(4-fluoro-3-(S-methylsulfonimidoyl)phenyl)-4-methyl-5-(1-methyl-1H-pyrazol-4-yl)nicotinamide and (R)-2-(4,4-difluoroazepan-1-yl)-N-(4-fluoro-3-(S-methylsulfonimidoyl)phenyl)-4-methyl-5-(1-methyl-1H-pyrazol-4-yl)nicotinamide

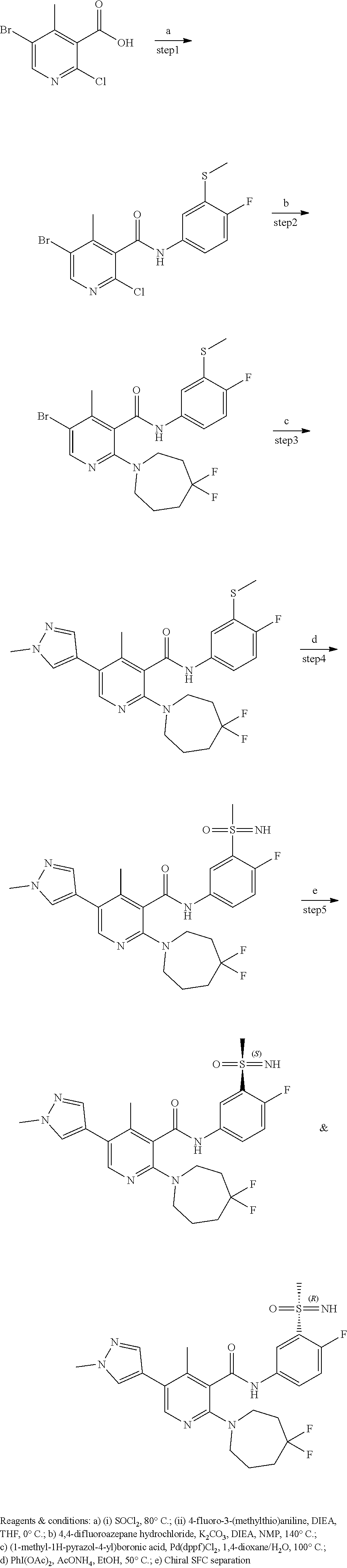

Step 1: 5-bromo-2-chloro-N-(4-fluoro-3-(methylthio)phenyl)-4-methylnicotinamide: A solution of 5-bromo-2-chloro-4-methylnicotinic acid (500 mg, 2.0 mmol) in SOCl₂ (6 mL) was heated to 80° C. for 1 hour. The solution was concentrated under vacuum to provide the chloride intermediate. Then the chloride intermediate dissolved in THF (5 mL) was added to a solution of 4-fluoro-3-(methylthio)aniline (313.8 mg, 2.0 mmol) and DIEA (1.3 g, 10.0 mmol) in THF (5 mL) at 0° C. The resulting mixture was stirred at 25° C. for 2 hours. Then the mixture was quenched with water (20 mL) and extracted with DCM (20 mL×3). The combined organic layers were washed with brine, dried over sodium sulfate, concentrated under vacuum. The residue was purified by flash column chromatography on silica gel (PE/EtOAc=1/1) to provide 5-bromo-2-chloro-N-(4-fluoro-3-(methylthio)phenyl)-4-methylnicotinamide (500 mg, 64.4%) as a white solid. LCMS (ESI) calcd. for C₁₄H₁₂BrClFN₂OS [M+H]⁺ m/z 388.95, found 390.8.

Step 2: 5-bromo-2-(4,4-difluoroazepan-1-yl)-N-(4-fluoro-3-(methylthio)phenyl)-4-methylnicotinamide: A solution of 5-bromo-2-chloro-N-(4-fluoro-3-(methylthio)phenyl)-4-methylnicotinamide (500 mg, 1.28 mmol), 4,4-difluoroazepane hydrochloride (330 mg, 1.92 mmol), K₂CO₃ (532 mg, 3.85 mmol) and DIEA (497.5 mg, 3.85 mmol) in NMP (8 mL) was heated to 140° C. and refluxed for 16 hours. After the reaction was completed, the mixture was cooled to room temperature. The resulting solution was diluted with EtOAc (30 mL) and washed with water (30 mL×3). The combined organic layers were washed with brine, dried over sodium sulfate, concentrated under vacuum. The residue was purified by flash column chromatography on silica gel (PE/EtOAc=1/2) to provide 5-bromo-2-(4,4-difluoroazepan-1-yl)-N-(4-fluoro-3-(methylthio)phenyl)-4-methylnicotinamide (260 mg, 41.7%) as a white solid. LCMS (ESI) calcd. for C₂₀H₂₂BrF₃N₃OS [M+H]⁺ m/z 490.06, found 490.0.

Step 3: 2-(4,4-difluoroazepan-1-yl)-N-(4-fluoro-3-(methylthio)phenyl)-4-methyl-5-(1-methyl-1H-pyrazol-4-yl)nicotinamide: A solution of 5-bromo-2-(4,4-difluoroazepan-1-yl)-N-(4-fluoro-3-(methylthio)phenyl)-4-methylnicotinamide (260 mg, 0.53 mmol), (1-methyl-1H-pyrazol-4-yl)boronic acid (134.1 mg, 1.06 mmol), K₂CO₃ (220.7 mg, 1.6 mmol) and Pd(dppf)Cl₂ (39 mg, 0.05 mmol) in 1,4-dioxane/H₂O (4/1, 10 mL) was heated to 100° C. and stirred for 16 hours under an atmosphere of N₂. After the reaction was completed, the mixture was cooled to room temperature. The resulting solution was diluted with water (10 mL) and extracted with EtOAc (10 mL×3). The combine organic layers were washed with brine, dried over Na₂SO₄, concentrated under vacuum. The residue was purified by flash column chromatography on silica gel (PE/EtAOc=1/1 to 1/3) to give 2-(4,4-difluoroazepan-1-yl)-N-(4-fluoro-3-(methylthio)phenyl)-4-methyl-5-(1-methyl-1H-pyrazol-4-yl)nicotinamide (200 mg, 77.1%) as a yellow solid. LCMS (ESI) calcd. for C₂₄H₂₇F₃N₅OS [M+H]⁺ m/z 490.19, found 490.1.

Step 4: 2-(4,4-difluoroazepan-1-yl)-N-(4-fluoro-3-(S-methylsulfonimidoyl)phenyl)-4-methyl-5-(1-methyl-1H-pyrazol-4-yl)nicotinamide: A solution of 2-(4,4-difluoroazepan-1-yl)-N-(4-fluoro-3-(methylthio)phenyl)-4-methyl-5-(1-methyl-1H-pyrazol-4-yl)nicotinamide (200 mg, 0.41 mmol), PhI(OAc)₂ (394.7 mg, 1.2 mmol), and AcONH₄ (63 mg, 0.82 mmol) in EtOH (6 mL) was heated to 50° C. and stirred for 2 hours. After the reaction was completed, the mixture was cooled to room temperature. The resulting solution was diluted with water (20 mL) and extracted with EtOAc (20 mL×3). The combine organic layers were washed with brine, dried over Na₂SO₄, concentrated under vacuum. The residue was purified by prep-HPLC (Gemini 5 um C₁₈ column, 150*21.2 mm, eluting with 25% to 80% MeCN/H₂O containing 0.05% NH₃) give 2-(4,4-difluoroazepan-1-yl)-N-(4-fluoro-3-(S-methylsulfonimidoyl)phenyl)-4-methyl-5-(1-methyl-1H-pyrazol-4-yl)nicotinamide (80 mg, 37.7%) as a white solid. LCMS (ESI) calcd. for C₂₄H₂₈F₃N₆O₂S [M+H]⁺ m/z 521.19, found 521.1.

Step 5: (S)-2-(4,4-difluoroazepan-1-yl)-N-(4-fluoro-3-(S-methylsulfonimidoyl)phenyl)-4-methyl-5-(1-methyl-1H-pyrazol-4-yl)nicotinamide and (R)-2-(4,4-difluoroazepan-1-yl)-N-(4-fluoro-3-(S-methylsulfonimidoyl)phenyl)-4-methyl-5-(1-methyl-1H-pyrazol-4-yl)nicotinamide: 2-(4,4-difluoroazepan-1-yl)-N-(4-fluoro-3-(S-methylsulfonimidoyl)phenyl)-4-methyl-5-(1-methyl-1H-pyrazol-4-yl)nicotinamide was further purified by Chiral-Prep-SFC (DAICEL OJ-H, 20 mm I.D.*250 mm L, 5 m, eluting with 75/25 CO₀₂/MeOH containing 0.1% NH₃) to give and (S)-2-(4,4-difluoroazepan-1-yl)-N-(4-fluoro-3-(S-methylsulfonimidoyl)phenyl)-4-methyl-5-(1-methyl-1H-pyrazol-4-yl)nicotinamide (35.3 mg) as the first elution. ¹H NMR (400 MHz, DMSO-d₆) 10.85 (s, 1H), 8.31 (dd, J=6.5, 2.7 Hz, 1H), 8.17 (s, 1H), 7.98-7.91 (m, 1H), 7.89 (s, 1H), 7.60 (s, 1H), 7.44 (t, J=9.3 Hz, 1H), 4.70 (s, 1H), 3.88 (s, 3H), 3.63-3.52 (m, 4H), 3.19 (s, 3H), 2.32-2.20 (m, 5H), 2.07-1.94 (m, 2H), 1.84-1.74 (m, 2H). LCMS (ESI) calcd. for C₂₄H₂₈F₃N₆O₂S [M+H]⁺ m/z 521.19, found 521.1 and (R)-2-(4,4-difluoroazepan-1-yl)-N-(4-fluoro-3-(S-methylsulfonimidoyl)phenyl)-4-methyl-5-(1-methyl-1H-pyrazol-4-yl)nicotinamide (30.1 mg) as the second elution. ¹H NMR (400 MHz, DMSO-d₆) 10.85 (s, 1H), 8.32 (d, J=6.4 Hz, 1H), 8.17 (d, J=2.2 Hz, 1H), 7.98-7.91 (m, 1H), 7.89 (s, 1H), 7.60 (s, 1H), 7.44 (t, J=9.3 Hz, 1H), 4.71 (s, 1H), 3.89 (d, J=2.1 Hz, 3H), 3.65-3.51 (m, 4H), 3.20 (s, 3H), 2.35-2.18 (m, 5H), 2.06-1.93 (m, 2H), 1.85-1.74 (m, 2H). LCMS (ESI) calcd. for C₂₄H₂₈F₃N₆O₂S [M+H]⁺ m/z 521.19, found 521.2.

### Example 215

### 5-chloro-N-(3-((R)—S-methylsulfonimidoyl)phenyl)-4-(trifluoromethyl)-2-((S)-2-(trifluoromethyl)morpholino)benzamide

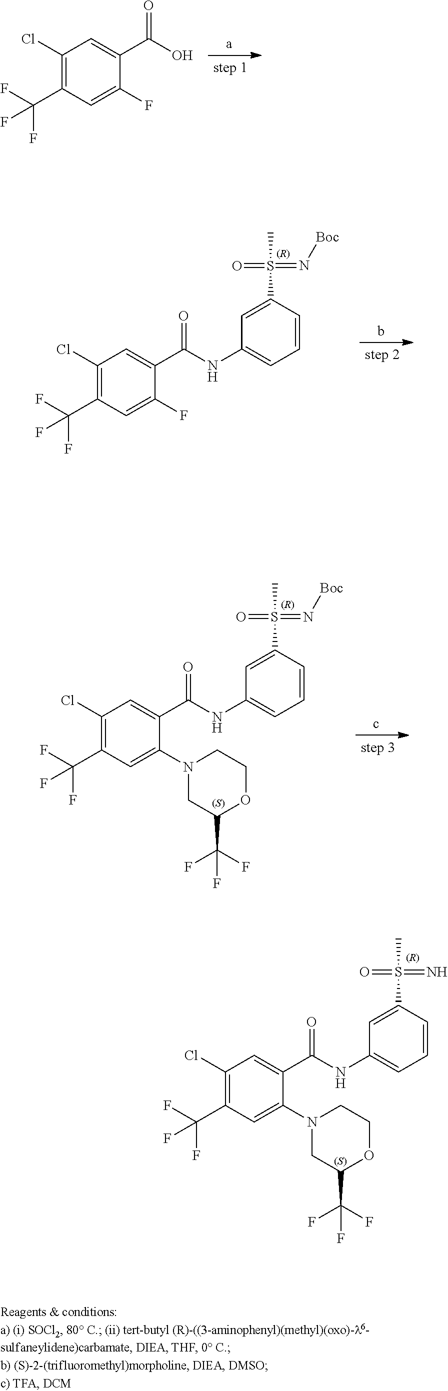

Step 1: tert-butyl (R)-((3-(5-chloro-2-fluoro-4-(trifluoromethyl)benzamido)phenyl)(methyl)(oxo)-λ⁶-sulfaneylidene)carbamate: A solution of 5-chloro-2-fluoro-4-(trifluoromethyl)benzoic acid (600 mg, 2.47 mmol) in SOCl₂ (6 mL) was heated to 80° C. and stirred for 0.5 hour. The solution was concentrated under vacuum to provide the chloride intermediate. Then the chloride intermediate in THE (2 mL) was added to a stirred solution of tert-butyl (R)-((3-aminophenyl)(methyl)(oxo)-λ⁶-sulfaneylidene)carbamate (738 mg, 2.72 mmol) and DIEA (1.59 g, 12.37 mmol) in THE (10 mL) at 0° C. The resulting mixture was stirred at 25° C. for 1 hour. Then the mixture was quenched with water (30 mL) and extracted with EtOAc (30 mL×3). The combined organic layers were washed with brine, dried over sodium sulfate, concentrated under vacuum. The residue was purified by flash column chromatography on silica gel (PE/EtOAc=3/2) to provide tert-butyl (R)-((3-(5-chloro-2-fluoro-4-(trifluoromethyl)benzamido)phenyl)(methyl)(oxo)-λ⁶-sulfaneylidene)carbamate (550 mg, 44.89%) as a white solid. LCMS (ESI) calcd. for C₂₀H₁₉ClF₄N₂O₄SNa [M+Na]m/z 517.06, found 516.8.

Step 2: tert-butyl ((R)-(3-(5-chloro-4-(trifluoromethyl)-2-((S)-2-(trifluoromethyl)morpholino)benzamido)phenyl)(methyl)(oxo)-λ⁶-sulfaneylidene)carbamate: A mixture of tert-butyl (R)-((3-(5-chloro-2-fluoro-4-(trifluoromethyl)benzamido)phenyl)(methyl)(oxo)-λ⁶-sulfaneylidene)carbamate (150 mg, 0.30 mmol) and (S)-2-(trifluoromethyl)morpholine (116 mg, 0.61 mmol) in DMSO (2 mL) was added DIEA (391 mg, 3.02 mmol) at room temperature. The reaction mixture was heated to 100° C. and stirred for 24 hours. After the reaction was completed, the resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtAOc=1/1) to give tert-butyl ((R)-(3-(5-chloro-4-(trifluoromethyl)-2-((S)-2-(trifluoromethyl)morpholino)benzamido)phenyl)(methyl)(oxo)-λ⁶-sulfaneylidene)carbamate (80 mg, 41.9%) as a yellow solid. LCMS (ESI) calcd. for C₂₅H₂₇ClF₆N₃O₅S [M+H]⁺ m/z 630.13, found 630.1.

Step 3: 5-chloro-N-(3-((R)—S-methylsulfonimidoyl)phenyl)-4-(trifluoromethyl)-2-((S)-2-(trifluoromethyl)morpholino)benzamide: A solution of tert-butyl ((R)-(3-(5-chloro-4-(trifluoromethyl)-2-((S)-2-(trifluoromethyl)morpholino)benzamido)phenyl)(methyl)(oxo)-λ⁶-sulfaneylidene)carbamate (80 mg, 0.13 mmol) in DCM (3 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 80% MeCN/H₂O containing 0.05% NH₃) to provide 5-chloro-N-(3-((R)—S-methylsulfonimidoyl)phenyl)-4-(trifluoromethyl)-2-((S)-2-(trifluoromethyl)morpholino)benzamide (23.4 mg, 34.8%) as a white solid. ¹H NMR (400 MHz, DMSO-d₆) δ 10.87 (s, 1H), 8.39 (s, 1H), 7.92 (d, J=7.9 Hz, 1H), 7.85 (s, 1H), 7.70 (d, J=7.7 Hz, 1H), 7.65-7.57 (m, 2H), 4.24 (s, 1H), 4.20-4.11 (m, 1H), 4.06-3.96 (m, 1H), 3.64-3.53 (m, 1H), 3.48-3.41 (m, 1H), 3.23-3.08 (m, 3H), 3.06 (s, 3H). LCMS (ESI) calcd. for C₂₀H₁₉ClF₆N₃O₃S [M+H]⁺ m/z 530.07, found 530.0.

### Example 216

### 5-chloro-N-(3-((R)—S-methylsulfonimidoyl)phenyl)-4-(trifluoromethyl)-2-((R)-2-(trifluoromethyl)morpholino)benzamide

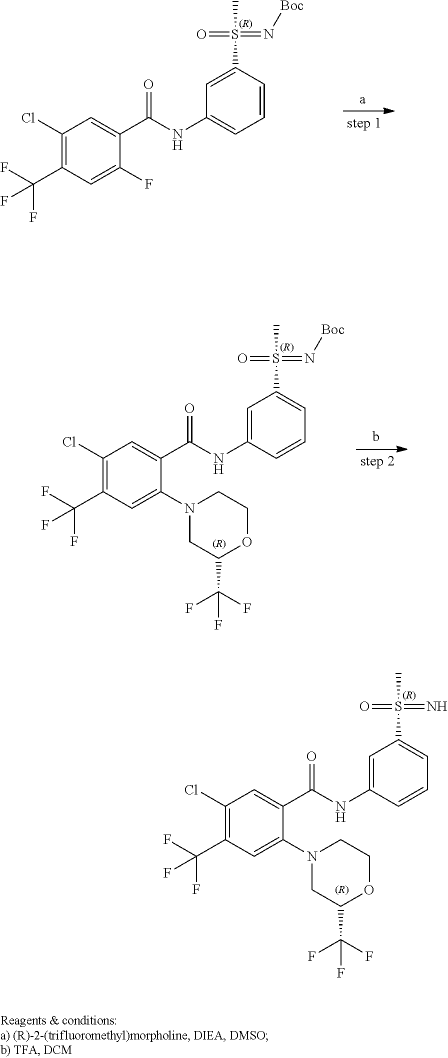

Step 1: tert-butyl ((R)-(3-(5-chloro-4-(trifluoromethyl)-2-((R)-2-(trifluoromethyl)morpholino)benzamido)phenyl)(methyl)(oxo)-λ⁶-sulfaneylidene)carbamate: A mixture of tert-butyl (R)-((3-(5-chloro-2-fluoro-4-(trifluoromethyl)benzamido)phenyl)(methyl)(oxo)-λ⁶-sulfaneylidene)carbamate (150 mg, 0.30 mmol) and (R)-2-(trifluoromethyl)morpholine (124 mg, 0.80 mmol) in DMSO (2 mL) was added DIEA (547 mg, 4.24 mmol) at room temperature. The reaction mixture was heated to 100° C. and stirred for 24 hours. After the reaction was completed, the resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtAOc=1/1) to give tert-butyl ((R)-(3-(5-chloro-4-(trifluoromethyl)-2-((R)-2-(trifluoromethyl)morpholino)benzamido)phenyl)(methyl)(oxo)-λ⁶-sulfaneylidene)carbamate (70 mg, 37.1%) as a yellow solid. LCMS (ESI) calcd. for C₂₅H₂₇ClF₆N₃O₅S [M+H]⁺ m/z 630.13, found 630.0.

Step 2: 5-chloro-N-(3-((R)—S-methylsulfonimidoyl)phenyl)-4-(trifluoromethyl)-2-((R)-2-(trifluoromethyl)morpholino)benzamide: A solution of tert-butyl ((R)-(3-(5-chloro-4-(trifluoromethyl)-2-((R)-2-(trifluoromethyl)morpholino)benzamido)phenyl)(methyl)(oxo)-λ⁶-sulfaneylidene)carbamate (70 mg, 0.11 mmol) in DCM (3 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 80% MeCN/H₂O containing 0.05% NH₃) to provide 5-chloro-N-(3-((R)—S-methylsulfonimidoyl)phenyl)-4-(trifluoromethyl)-2-((R)-2-(trifluoromethyl)morpholino)benzamide (26.2 mg, 44.5%) as a white solid. ¹H NMR (400 MHz, DMSO-d₆) δ 10.87 (s, 1H), 8.39 (s, 1H), 7.91 (d, J=8.0 Hz, 1H), 7.85 (s, 1H), 7.70 (d, J=7.8 Hz, 1H), 7.66-7.57 (m, 2H), 4.24 (s, 1H), 4.21-4.13 (m, 1H), 4.13-3.95 (m, 1H), 3.69-3.49 (m, 1H), 3.49-3.38 (m, 1H), 3.20-3.03 (m, 6H). LCMS (ESI) calcd. for C₂₀H₁₉ClF₆N₃O₃S [M+H]⁺ m/z 530.07, found 530.0.

### Example 217

### 5-chloro-N-(2-((R)—S-methylsulfonimidoyl)pyridin-4-yl)-4-(trifluoromethyl)-2-((R)-2-(trifluoromethyl)morpholino)benzamide

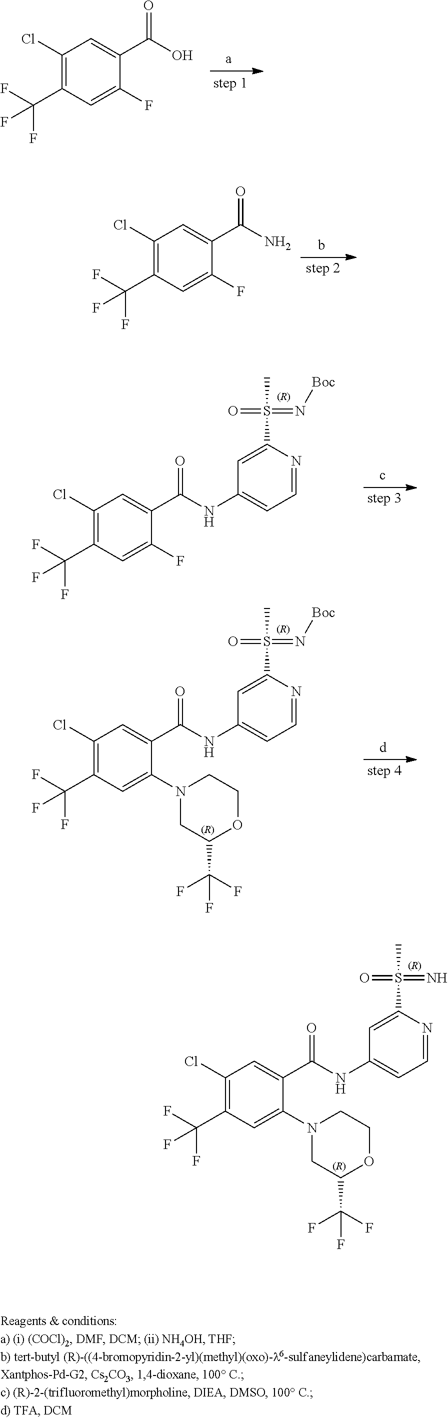

Step 1: 5-chloro-2-fluoro-4-(trifluoromethyl)benzamide: A solution of 5-chloro-2-fluoro-4-(trifluoromethyl)benzoic acid (500 mg, 2.07 mmol) in DCM (10 mL) was added DMF (2 drops) and oxalyl chloride (0.7 mL) at room temperature. After addition, the reaction mixture was stirred at room temperature for 1 hour. After the reaction was completed, the reaction mixture was concentrated. The residue was dissolved in THF (10 mL) and added dropwise to a stirred solution of ammonium hydroxide (5 mL). Then the mixture was stirred at room temperature for 1 hour. After the reaction was completed, the mixture was diluted with water (10 mL) and extracted with EtOAc (10 mL×3). The combine organic layers were washed with brine, dried over sodium sulfate, concentrated under vacuum. The residue was purified by flash column chromatography on silica gel (PE/EtOAc=1/1 to 1/2) to give 5-chloro-2-fluoro-4-(trifluoromethyl)benzamide (450 mg, 90.36%) as a white solid.

Step 2: tert-butyl (R)-((4-(5-chloro-2-fluoro-4-(trifluoromethyl)benzamido)pyridin-2-yl)(methyl)(oxo)-λ⁶-sulfaneylidene)carbamate: A solution of 5-chloro-2-fluoro-4-(trifluoromethyl)benzamide (450 mg, 1.86 mmol) in 1,4-dioxane (10 mL) was added tert-butyl (R)-((4-bromopyridin-2-yl)(methyl)(oxo)-λ⁶-sulfaneylidene)carbamate (745 mg, 2.23 mmol), Cs₂CO₃ (1.8 g, 5.58 mmol) and Xantphos-Pd-G2 (165 mg, 0.18 mol) at room temperature under nitrogen. Then the reaction mixture was heated to 100° C. and stirred for 16 hours under nitrogen. The reaction was monitored by LCMS. After the reaction was completed, the filtrate was diluted with water (20 mL) and extracted with DCM (20 mL×3). The combine organic layers were washed with brine, dried over sodium sulfate, concentrated under vacuum. The residue was purified by flash column chromatography on silica gel (PE/EtOAc=1/1) to give tert-butyl (R)-((4-(5-chloro-2-fluoro-4-(trifluoromethyl)benzamido)pyridin-2-yl)(methyl)(oxo)-λ⁶-sulfaneylidene)carbamate (500 mg, 54.23%) as a yellow solid. LCMS (ESI) calcd. for C₁₉H₁₉ClF₄N₃O₄S [M+H]⁺ m/z 496.07, found 495.9.

Step 3: tert-butyl ((R)-(4-(5-chloro-4-(trifluoromethyl)-2-((R)-2-(trifluoromethyl)morpholino)benzamido)pyridin-2-yl)(methyl)(oxo)-λ⁶-sulfaneylidene)carbamate: A mixture of tert-butyl (R)-((4-(5-chloro-2-fluoro-4-(trifluoromethyl)benzamido)pyridin-2-yl)(methyl)(oxo)-λ⁶-sulfaneylidene)carbamate (150 mg, 0.30 mmol) and (R)-2-(trifluoromethyl)morpholine (116 mg, 0.61 mmol) in DMSO (2 mL) was added DIEA (391 mg, 3.02 mmol) at room temperature. Then the reaction mixture was heated to 100° C. and stirred for 24 hours. After the reaction was completed, the resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtAOc=1/1 to 1/2) to give tert-butyl ((R)-(4-(5-chloro-4-(trifluoromethyl)-2-((R)-2-(trifluoromethyl)morpholino)benzamido)pyridin-2-yl)(methyl)(oxo)-16-sulfaneylidene)carbamate (70 mg, 36.6%) as a yellow solid. LCMS (ESI) calcd. for C₂₄H₂₆ClF₆N₄O₅S [M+H]⁺ m/z 631.12, found 631.1.

Step 4: 5-chloro-N-(2-((R)—S-methylsulfonimidoyl)pyridin-4-yl)-4-(trifluoromethyl)-2-((R)-2-(trifluoromethyl)morpholino)benzamide: A solution of tert-butyl ((R)-(4-(5-chloro-4-(trifluoromethyl)-2-((R)-2-(trifluoromethyl)morpholino)benzamido)pyridin-2-yl)(methyl)(oxo)-16-sulfaneylidene)carbamate (70 mg, 0.11 mmol) in DCM (3 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 80% MeCN/H₂O containing 0.05% NH₃) to provide 5-chloro-N-(2-((R)—S-methylsulfonimidoyl)pyridin-4-yl)-4-(trifluoromethyl)-2-((R)-2-(trifluoromethyl)morpholino)benzamide (17.8 mg, 30.2%) as a white solid. ¹H NMR (400 MHz, DMSO-d₆) δ 11.17 (s, 1H), 8.62 (d, J=5.3 Hz, 1H), 8.43 (s, 1H), 7.91-7.76 (m, 2H), 7.61 (s, 1H), 4.37 (s, 1H), 4.23-4.12 (m, 1H), 4.03-3.95 (m, 1H), 3.60-3.50 (m, 1H), 3.46-3.40 (m, 1H), 3.15 (s, 3H), 3.14-2.99 (m, 3H). LCMS (ESI) calcd. for C₁₉H₁₈ClF₆N₄O₃S [M+H]⁺ m/z 531.07, found 531.1.

### Example 218

### 5-chloro-N-(2-((R)—S-methylsulfonimidoyl)pyridin-4-yl)-4-(trifluoromethyl)-2-((S)-2-(trifluoromethyl)morpholino)benzamide

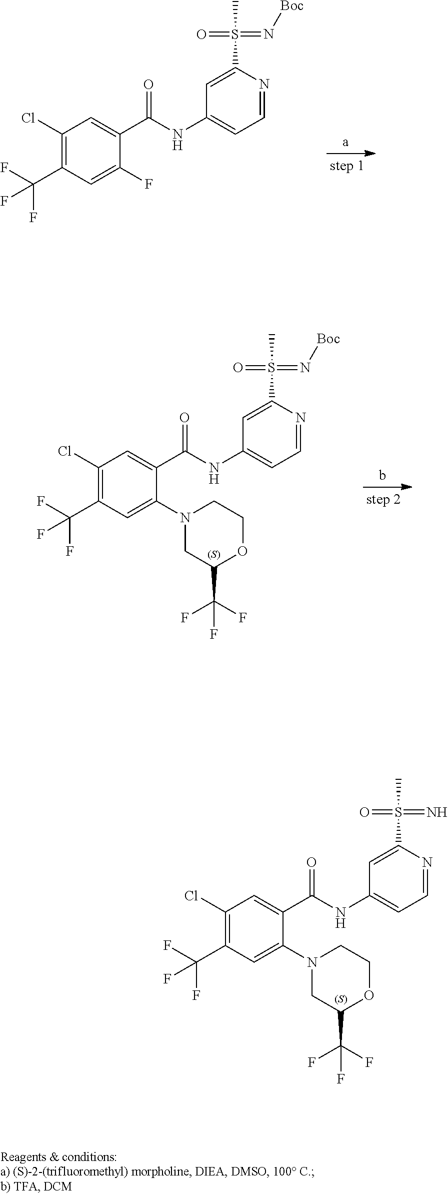

Step 1: tert-butyl ((R)-(4-(5-chloro-4-(trifluoromethyl)-2-((S)-2-(trifluoromethyl)morpholino)benzamido)pyridin-2-yl)(methyl)(oxo)-λ⁶-sulfaneylidene)carbamate: A mixture of tert-butyl (R)-((4-(5-chloro-2-fluoro-4-(trifluoromethyl)benzamido)pyridin-2-yl)(methyl)(oxo)-λ⁶-sulfaneylidene)carbamate (150 mg, 0.30 mmol) and (S)-2-(trifluoromethyl)morpholine hydrochloride (116 mg, 0.61 mmol) in DMSO (2 mL) was added DIEA (391 mg, 3.02 mmol) at room temperature. The reaction mixture was heated to 100° C. and stirred for 24 hours. After the reaction was completed, the resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtAOc=1/1) to give tert-butyl ((R)-(4-(5-chloro-4-(trifluoromethyl)-2-((S)-2-(trifluoromethyl)morpholino)benzamido)pyridin-2-yl)(methyl)(oxo)-λ⁶-sulfaneylidene)carbamate (70 mg, 36.6%) as a yellow solid. LCMS (ESI) calcd. for C₂₄H₂₆ClF₆N₄O₅S [M+H]⁺ m/z 631.12, found 631.1.

Step 2: 5-chloro-N-(2-((R)—S-methylsulfonimidoyl)pyridin-4-yl)-4-(trifluoromethyl)-2-((S)-2-(trifluoromethyl)morpholino)benzamide. A solution of tert-butyl ((R)-(4-(5-chloro-4-(trifluoromethyl)-2-((S)-2-(trifluoromethyl)morpholino)benzamido)pyridin-2-yl)(methyl)(oxo)-λ⁶-sulfaneylidene)carbamate (70 mg, 0.11 mmol) in DCM (3 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 60% to 90% MeCN/H₂O containing 0.05% NH₃·H₂O) to provide 5-chloro-N-(2-((R)—S-methylsulfonimidoyl)pyridin-4-yl)-4-(trifluoromethyl)-2-((S)-2-(trifluoromethyl)morpholino)benzamide (12.5 mg, 21.2%) as a white solid. ¹H NMR (400 MHz, DMSO-d₆) δ 11.24 (s, 1H), 8.64 (d, J=5.4 Hz, 1H), 8.45 (s, 1H), 7.96-7.75 (m, 2H), 7.62 (s, 1H), 4.38 (s, 1H), 4.25-4.11 (m, 1H), 4.05-3.93 (m, 1H), 3.62-3.51 (m, 1H), 3.47-3.38 (m, 1H), 3.15 (s, 3H), 3.14-2.98 (m, 3H). LCMS (ESI) calcd. for C₁₉H₁₈ClF₆N₄O₃S [M+H]⁺ m/z 531.07, found 531.0.

### Example 219

### (R)-5-chloro-N-(3-(S-methylsulfonimidoyl)phenyl)-4-(trifluoromethyl)-2-(4-(trifluoromethyl)piperidin-1-yl)benzamide

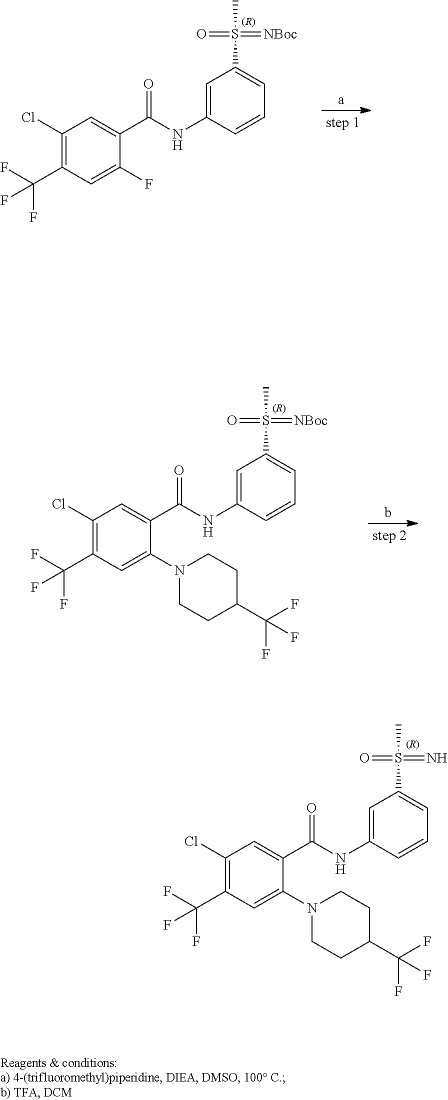

Step 1: tert-butyl (R)-((3-(5-chloro-4-(trifluoromethyl)-2-(4-(trifluoromethyl)piperidin-1-yl)benzamido)phenyl)(methyl)(oxo)-λ⁶-sulfaneylidene)carbamate: A solution of tert-butyl (R)-((3-(5-chloro-2-fluoro-4-(trifluoromethyl)benzamido)phenyl)(methyl)(oxo)-λ⁶-sulfaneylidene)carbamate (150 mg, 0.30 mmol), 4-(trifluoromethyl)piperidine (46 mg, 0.30 mmol) and DIEA (157 mg, 1.21 mmol) in DMSO (10 mL) was heated to 100° C. and stirred for 16 hours. LCMS showed the reaction was completed. The mixture was diluted with water (20 mL) and extracted with EtOAc (20 mL×3). The combined organic layers were washed with brine, dried over sodium sulfate, concentrated under vacuum. The residue was purified by flash column chromatography on silica gel (PE/EtOAc=2/1) to provide tert-butyl (R)-((3-(5-chloro-4-(trifluoromethyl)-2-(4-(trifluoromethyl)piperidin-1-yl)benzamido)phenyl)(methyl)(oxo)-λ⁶-)carbamate (100 mg, 52.52%) as yellow oil. LCMS (ESI) calcd. for C₂₆H₂₉ClF₆N₃O₄S [M+H]⁺ m/z 628.15, found 628.1.

Step 2: (R)-5-chloro-N-(3-(S-methylsulfonimidoyl)phenyl)-4-(trifluoromethyl)-2-(4-(trifluoromethyl)piperidin-1-yl)benzamide: A solution of tert-butyl (R)-((3-(5-chloro-4-(trifluoromethyl)-2-(4-(trifluoromethyl)piperidin-1-yl)benzamido)phenyl)(methyl)(oxo)-λ⁶-sulfaneylidene)carbamate (100 mg, 0.16 mmol) in DCM (5 mL) was added TFA (0.5 mL) at room temperature. The reaction mixture was stirred at room temperature for 1 hour. After the reaction was completed, the mixture was concentrated and the residue was adjusted to pH=8-9 with saturated aqueous NaHCO₃. Then the aqueous solution was extracted with DCM (10 mL*3). The combined organic layers were washed with brine, dried over sodium sulfate, concentrated under vacuum. The residue was purified by prep-HPLC (Gemini 5 um C₁₈ column, 150*21.2 mm, eluting with 40% to 60% MeCN/H₂O containing 0.05% NH₃) to provide (R)-5-chloro-N-(3-(S-methylsulfonimidoyl)phenyl)-4-(trifluoromethyl)-2-(4-(trifluoromethyl)piperidin-1-yl)benzamide (39.6 mg, 51.30%). ¹H NMR (400 MHz, DMSO-d₆) δ 10.92 (s, 1H), 8.42 (s, 1H), 7.89-7.82 (m, 2H), 7.70-7.58 (m, 2H), 7.47 (s, 1H), 4.21 (s, 1H), 3.42-3.32 (m, 2H), 3.04 (s, 3H), 2.93-2.84 (m, 2H), 2.46-2.35 (m, 1H), 1.89-1.81 (m, 2H), 1.61-1.43 (m, 2H). LCMS (ESI) calcd. for C₂₁H₂₁ClF₆N₃O₂S [M+H]⁺ m/z 528.09, found 528.0.

### Example 220

### (R)-6-cyclopropyl-2-(4,4-difluoroazepan-1-yl)-4-methyl-N-(2-(S-methylsulfonimidoyl)pyridin-4-yl)-5-(trifluoromethyl)nicotinamide

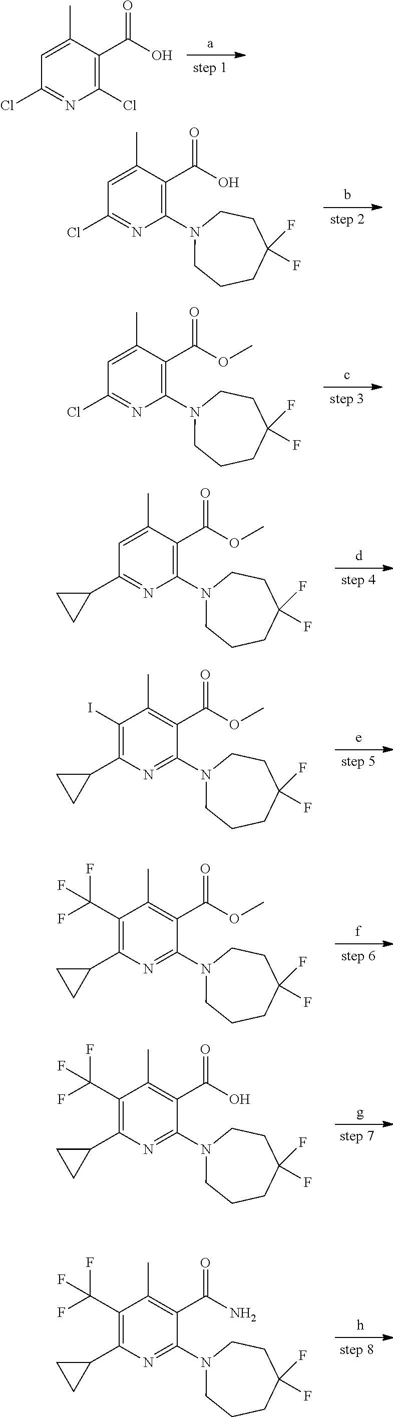

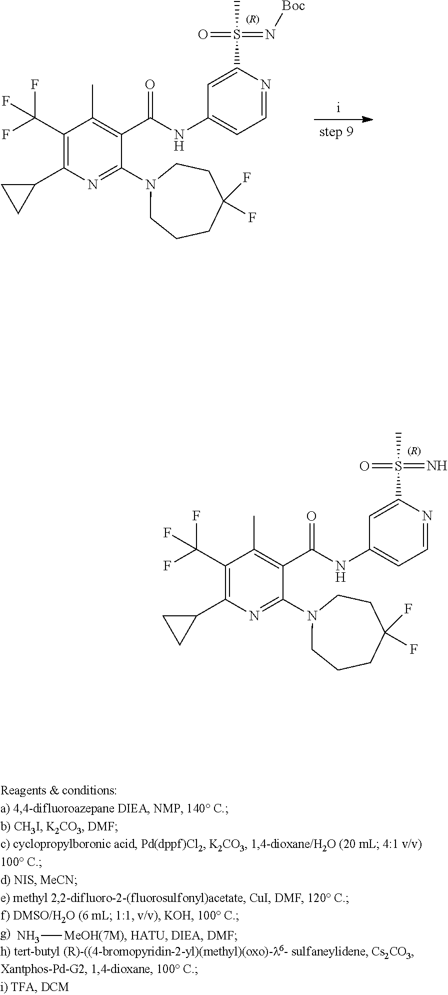

Step 1: 6-chloro-2-(4,4-difluoroazepan-1-yl)-4-methylnicotinic acid: A mixture of 2,6-dichloro-4-methylnicotinic acid (1 g, 4.88 mmol) and 4,4-difluoroazepane (790 mg, 5.86 mmol) in NMP (20 mL) was added DIEA (3.15 g, 24.4 mmol) at room temperature. The mixture was heated to 140° C. and stirred for 6 hours. The reaction was monitored by LCMS. After the reaction was completed, the resulting solution was cooled and diluted with water (40 mL) and extracted with EtOAc (30 mL×3). The combined organic layers were washed with brine, dried over Na₂SO₄, concentrated under vacuum. The residue was purified by flash column chromatography on silica gel (DCM/MeOH=20/1 to 10/1) to give 6-chloro-2-(4,4-difluoroazepan-1-yl)-4-methylnicotinic acid (1 g, 67.1%) as a yellow solid. LCMS (ESI) calcd. C₁₃H₁₆ClF₂N₂O₂ for [M+H]⁺ m/z 305.09, found 305.1.

Step 2: methyl 6-chloro-2-(4,4-difluoroazepan-1-yl)-4-methylnicotinate: A solution of 6-chloro-2-(4,4-difluoroazepan-1-yl)-4-methylnicotinic acid (1 g, 3.29 mmol) in DMF (20 mL) was added K₂CO₃ (1.36 g, 9.87 mmol) and CH₃I (701 mg, 4.94 mmol) at room temperature. The reaction mixture was stirred at room temperature for 3 hours. The reaction was monitored by LCMS. After the reaction was completed, the resulting solution was diluted with water (40 mL) and extracted with EtOAc (20 mL×3). The combined organic layers were washed with brine, dried over Na₂SO₄, concentrated under vacuum. The residue was purified by flash column chromatography on silica gel (PE/EtOAc=10/1 to 5/1) to give methyl 6-chloro-2-(4,4-difluoroazepan-1-yl)-4-methylnicotinate (800 mg, 76.5%) as yellow oil. LCMS (ESI) calcd. for C₁₄H₁₈ClF₂N₂O₂ [M+H]⁺ m/z 319.10, found 318.7.

Step 3: methyl 6-cyclopropyl-2-(4,4-difluoroazepan-1-yl)-4-methylnicotinate: A solution of methyl 6-chloro-2-(4,4-difluoroazepan-1-yl)-4-methylnicotinate (800 mg, 2.52 mmol), cyclopropylboronic acid (650 mg, 7.56 mmol), K₂CO₃ (1.39 g, 10.1 mmol) and Pd(dppf)Cl₂ (368 mg, 0.50 mmol) in 1,4-dioxane/H₂O=4:1 (20 mL) was stirred under nitrogen. The reaction mixture was stirred to 100° C. and stirred for 16 hours. The reaction was monitored by LCMS. After the reaction was completed, the resulting solution was cooled and diluted with water (40 mL) and extracted with EtOAc (30 mL×3). The combined organic layers were washed with brine, dried over Na₂SO₄, concentrated under vacuum. The residue was purified by flash column chromatography on silica gel (PE/EtOAc=10/1 to 4/1) to give methyl 6-cyclopropyl-2-(4,4-difluoroazepan-1-yl)-4-methylnicotinate (500 mg, 61.2%) as yellow oil. LCMS (ESI) calcd. for C₁₇H₂₃F₂N₂O₂ [M+H]⁺ m/z 325.17, found 324.8.

Step 4: methyl 6-cyclopropyl-2-(4,4-difluoroazepan-1-yl)-5-iodo-4-methylnicotinate: A solution of methyl 6-cyclopropyl-2-(4,4-difluoroazepan-1-yl)-4-methylnicotinate (500 mg, 1.54 mmol) in MeCN (10 mL) was added NIS (415.8 mg, 1.85 mmol) at 0° C. The reaction mixture was stirred at 25° C. for 16 hours. The reaction was monitored by LCMS. After the reaction was completed, the resulting solution was diluted with water (20 mL) and extracted with EtOAc (20 mL×3). The combined organic layers were washed with brine, dried over Na₂SO₄, concentrated under vacuum. The residue was purified by flash column chromatography on silica gel (PE/EtOAc=20/1 to 10/1) to give methyl 6-cyclopropyl-2-(4,4-difluoroazepan-1-yl)-5-iodo-4-methylnicotinate (400 mg, 57.8%) as yellow oil. LCMS (ESI) calcd. for C₁₇H₂₂F₂IN₂O₂ [M+H]⁺ m/z 451.07, found 450.8.

Step 5: methyl 6-cyclopropyl-2-(4,4-difluoroazepan-1-yl)-4-methyl-5-(trifluoromethyl)nicotinate: A solution of methyl 6-cyclopropyl-2-(4,4-difluoroazepan-1-yl)-5-iodo-4-methylnicotinate (400 mg, 0.89 mmol) and CuI (339 mg, 1.78 mmol) in DMF (10 ml) was added methyl 2,2-difluoro-2-(fluorosulfonyl)acetate (867.8 mg, 4.45 mmol) at room temperature. The reaction mixture was heated to 120° C. and stirred for 5 hours under N₂. After the reaction was completed, the resulting solution was cooled and diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtOAc=10/1 to 5/1) to give methyl 6-cyclopropyl-2-(4,4-difluoroazepan-1-yl)-4-methyl-5-(trifluoromethyl)nicotinate (250 mg, 71.7%) as yellow solid. LCMS (ESI) calcd. for C₁₈H₂₂F₅N₂O₂ [M+H]⁺ m/z 393.16, found 393.0.

Step 6: 6-cyclopropyl-2-(4,4-difluoroazepan-1-yl)-4-methyl-5-(trifluoromethyl)nicotinic acid: A solution of methyl 6-cyclopropyl-2-(4,4-difluoroazepan-1-yl)-4-methyl-5-(trifluoromethyl)nicotinate (250 mg, 0.64 mmol) in DMSO/H₂O=1/1 (6 mL) was added KOH (2.87 g, 51.2 mmol) at room temperature. The reaction mixture was heated to 100° C. and stirred for 1 hour. After the reaction was completed, the reaction mixture was adjusted to pH=3-4 with aqueous HCl (1M). Then the solution was extracted with EtOAc (30 mL×3). The combined organic layers were washed with brine, dried over Na₂SO₄, concentrated under vacuum to obtain 6-cyclopropyl-2-(4,4-difluoroazepan-1-yl)-4-methyl-5-(trifluoromethyl)nicotinic acid (180 mg, 74.2%) as a yellow solid which was used without further purification. LCMS (ESI) calcd. for C₁₇H₂₀F₅N₂O₂ [M+H]⁺ m/z 379.14, found 379.1.

Step 7: 6-cyclopropyl-2-(4,4-difluoroazepan-1-yl)-4-methyl-5-(trifluoromethyl) nicotinamide: A solution of 6-cyclopropyl-2-(4,4-difluoroazepan-1-yl)-4-methyl-5-(trifluoromethyl)nicotinic acid (180 mg, 0.48 mmol), HATU (328.3 mg, 0.86 mmol) and DIEA (309.6 mg, 2.4 mmol) in DMF (5 ml) was added NH₃-MeOH (7 M, 1 mL). The solution was stirred for 16 hours at room temperature. After the reaction was completed, the resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtOAc=5/1 to 1/1) to give 6-cyclopropyl-2-(4,4-difluoroazepan-1-yl)-4-methyl-5-(trifluoromethyl)nicotinamide (150 mg, 82.6%) as a yellow solid. LCMS (ESI) calcd. for C₁₇H₂₁F₅N₃O [M+H]⁺ m/z 378.16, found 377.8.

Step 8: tert-butyl (R)-((4-(6-cyclopropyl-2-(4,4-difluoroazepan-1-yl)-4-methyl-5-(trifluoromethyl)nicotinamido)pyridin-2-yl)(methyl)(oxo)-λ⁶-sulfaneylidene)carbamate: A solution of 6-cyclopropyl-2-(4,4-difluoroazepan-1-yl)-4-methyl-5-(trifluoromethyl)nicotinamide (150 mg, 0.40 mmol), Cs₂CO₃ (391 mg, 1.20 mmol), Xantphos-Pd-G2 (72 mg, 0.08 mmol) and tert-butyl (R)-((4-bromopyridin-2-yl)(methyl)(oxo)-λ⁶-sulfaneylidene)carbamate (200 mg, 0.60 mmol) in 1,4-dioxane (5 ml) was heated to 100° C. and stirred for 16 hours under N₂. After the reaction was completed, the resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtOAc=1/1 to 1/2) to give tert-butyl (R)-((4-(6-cyclopropyl-2-(4,4-difluoroazepan-1-yl)-4-methyl-5-(trifluoromethyl)nicotinamido)pyridin-2-yl)(methyl)(oxo)-λ⁶-sulfaneylidene)carbamate (130 mg, 82.6%) as yellow solid. LCMS (ESI) calcd. for C₂₈H₃₅F₅N₅O₄S [M+H]⁺ m/z 632.23, found 632.1.

Step 9: (R)-6-cyclopropyl-2-(4,4-difluoroazepan-1-yl)-4-methyl-N-(2-(S-methylsulfonimidoyl)pyridin-4-yl)-5-(trifluoromethyl)nicotinamide: A solution of tert-butyl (R)-((4-(6-cyclopropyl-2-(4,4-difluoroazepan-1-yl)-4-methyl-5-(trifluoromethyl)nicotinamido)pyridin-2-yl)(methyl)(oxo)-λ⁶-sulfaneylidene)carbamate (130 mg, 0.21 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (10 mL×3). The combined organic layers were washed with brine, dried over sodium sulfate, concentrated under vacuum. The residue was purified by prep-HPLC (Gemini 5 um C₁₈ column, 150*21.2 mm, eluting with 40% to 95% MeCN/H₂O containing 0.05% NH₃·H₂O) to obtain (R)-6-cyclopropyl-2-(4,4-difluoroazepan-1-yl)-4-methyl-N-(2-(S-methylsulfonimidoyl)pyridin-4-yl)-5-(trifluoromethyl)nicotinamide (38 mg, 33.9%) as a white solid. ¹H NMR (400 MHz, DMSO-d₆) δ 11.20 (s, 1H), 8.61 (d, J=5.4 Hz, 1H), 8.38 (d, J=1.8 Hz, 1H), 7.81 (dd, J=5.4, 1.9 Hz, 1H), 4.37 (s, 1H), 3.63 (s, 2H), 3.53-3.47 (m, 2H), 3.15 (s, 3H), 2.31-2.15 (m, 6H), 2.04-1.89 (m, 2H), 1.83-1.74 (m, 2H), 1.07 (s, 2H), 1.02-0.96 (m, 2H). LCMS (ESI) calcd. for C₂₃H₂₇F₅N₅O₂S [M+H]⁺ m/z 532.18, found 532.1.

### Example 221

### (S)-6-cyclopropyl-2-(4,4-difluoroazepan-1-yl)-4-methyl-N-(2-(S-methylsulfonimidoyl)pyridin-4-yl)-5-(trifluoromethyl)nicotinamide

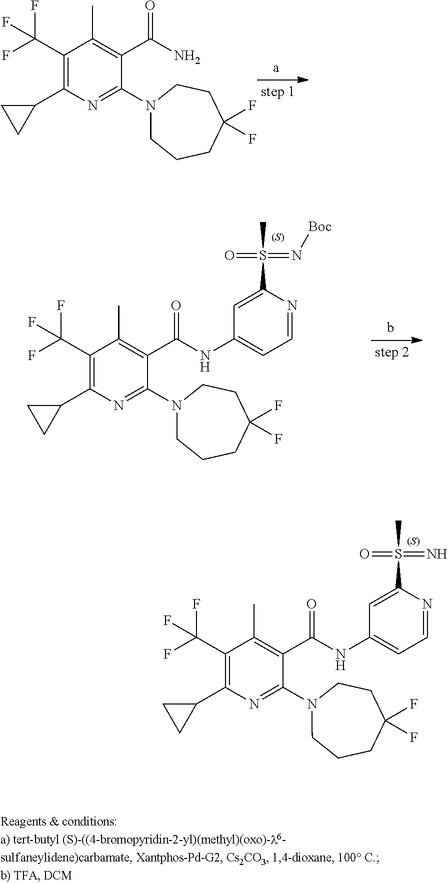

Step 1: tert-butyl (S)-((4-(6-cyclopropyl-2-(4,4-difluoroazepan-1-yl)-4-methyl-5-(trifluoromethyl)nicotinamido)pyridin-2-yl)(methyl)(oxo)-λ⁶-sulfaneylidene)carbamate: A solution of 6-cyclopropyl-2-(4,4-difluoroazepan-1-yl)-4-methyl-5-(trifluoromethyl)nicotinamide (150 mg, 0.40 mmol), Cs₂CO₃ (391 mg, 1.20 mmol), Xantphos-Pd-G2 (72 mg, 0.08 mmol) and tert-butyl (S)-((4-bromopyridin-2-yl)(methyl)(oxo)-λ⁶-sulfaneylidene)carbamate (200 mg, 0.60 mmol) in 1,4-dioxane (5 ml) was heated to 100° C. and stirred for 16 hours under N₂. After the reaction was completed, the resulting solution was cooled and diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtOAc=1/1) to give tert-butyl (S)-((4-(6-cyclopropyl-2-(4,4-difluoroazepan-1-yl)-4-methyl-5-(trifluoromethyl)nicotinamido)pyridin-2-yl)(methyl)(oxo)-λ⁶-sulfaneylidene)carbamate (130 mg, 82.6%) as a yellow solid. LCMS (ESI) calcd. for C₂₈H₃₅F₅N₅O₄S [M+H]⁺ m/z 632.24, found 632.05.

Step 2: (S)-6-cyclopropyl-2-(4,4-difluoroazepan-1-yl)-4-methyl-N-(2-(S-methylsulfonimidoyl)pyridin-4-yl)-5-(trifluoromethyl)nicotinamide: A solution of give tert-butyl (S)-((4-(6-cyclopropyl-2-(4,4-difluoroazepan-1-yl)-4-methyl-5-(trifluoromethyl)nicotinamido)pyridin-2-yl)(methyl)(oxo)-16-sulfaneylidene)carbamate (130 mg, 0.21 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (10 mL×3). The combined organic layers were washed with brine, dried over sodium sulfate, concentrated under vacuum. The residue was purified by prep-HPLC (Gemini 5 um C₁₈ column, 150*21.2 mm, eluting with 40% to 95% MeCN/H₂O containing 0.05% NH₃·H₂O) to obtain (S)-6-cyclopropyl-2-(4,4-difluoroazepan-1-yl)-4-methyl-N-(2-(S-methylsulfonimidoyl)pyridin-4-yl)-5-(trifluoromethyl)nicotinamide (50 mg, 44.8%) as a white solid. ¹H NMR (400 MHz, DMSO-d₆) δ 11.20 (s, 1H), 8.61 (d, J=5.4 Hz, 1H), 8.38 (d, J=1.8 Hz, 1H), 7.81 (dd, J=5.4, 2.0 Hz, 1H), 4.37 (s, 1H), 3.64 (s, 2H), 3.50 (s, 2H), 3.15 (s, 3H), 2.31-2.15 (m, 6H), 2.02-1.90 (m, 2H), 1.86-1.70 (m, 2H), 1.08 (s, 2H), 1.03-0.91 (m, 2H). LCMS (ESI) calcd. for C₂₃H₂₇F₅N₅O₂S [M+H]⁺ m/z 532.18, found 532.10.

### Example 222

### (R)-5-chloro-2-(4,4-difluoropiperidin-1-yl)-N-(3-(S-methylsulfonimidoyl)phenyl)-4-(trifluoromethyl)benzamide

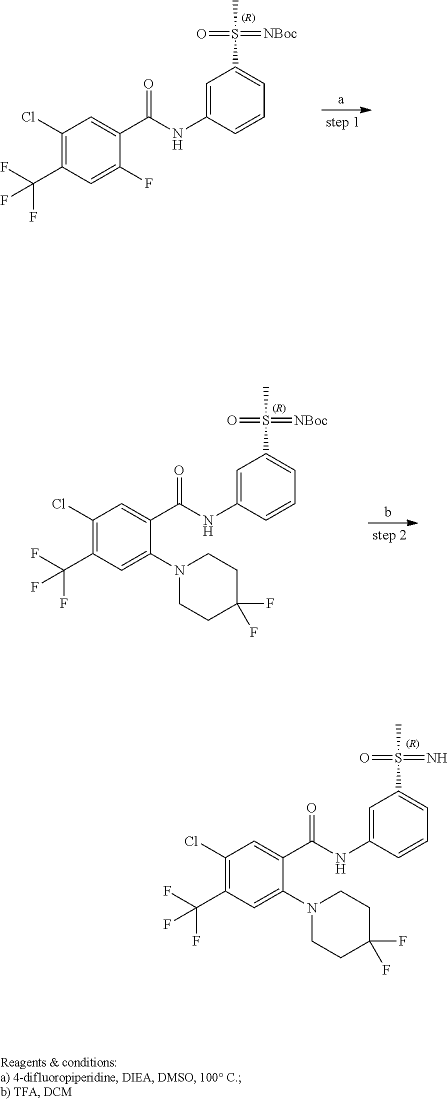

Step 1: tert-butyl (R)-((3-(5-chloro-2-(4,4-difluoropiperidin-1-yl)-4-(trifluoromethyl)benzamido)phenyl)(methyl)(oxo)-λ⁶-sulfaneylidene)carbamate: A solution of tert-butyl (R)-((3-(5-chloro-2-fluoro-4-(trifluoromethyl)benzamido)phenyl)(methyl)(oxo)-λ⁶-sulfaneylidene)carbamate (150 mg, 0.30 mmol), 4,4-difluoropiperidine (183 mg, 1.51 mmol) and DIEA (195 mg, 1.51 mmol) in DMSO (5 mL) was heated to 100° C. and stirred for 16 hours. LCMS showed the reaction was completed. The mixture was cooled and diluted with water (20 mL) and extracted with EtOAc (20 mL×3). The combined organic layers were washed with brine, dried over sodium sulfate, concentrated under vacuum. The residue was purified by flash column chromatography on silica gel (PE/EtOAc=2/1 to 1/1) to provide tert-butyl (R)-((3-(5-chloro-2-(4,4-difluoropiperidin-1-yl)-4-(trifluoromethyl)benzamido)phenyl)(methyl)(oxo)-λ⁶-sulfaneylidene)carbamate (130 mg, 71.9%) as yellow solid. LCMS (ESI) calcd. for C₂₅H₂₈ClF₅N₃O₄S [M+H]⁺ m/z 596.14, found 596.1.

Step 2: (R)-5-chloro-2-(4,4-difluoropiperidin-1-yl)-N-(3-(S-methylsulfonimidoyl)phenyl)-4-(trifluoromethyl)benzamide: A solution of tert-butyl (R)-((3-(5-chloro-2-(4,4-difluoropiperidin-1-yl)-4-(trifluoromethyl)benzamido)phenyl)(methyl)(oxo)-λ⁶-sulfaneylidene)carbamate (130 mg, 0.22 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 40% to 50% ACN/H₂O containing 0.1% TFA) to provide (R)-5-chloro-2-(4,4-difluoropiperidin-1-yl)-N-(3-(S-methylsulfonimidoyl)phenyl)-4-(trifluoromethyl)benzamide (65 mg, 60.0%) as a white solid. ¹H NMR (400 MHz, DMSO-d₆) δ 10.87 (s, 1H), 8.41 (d, J=1.7 Hz, 1H), 7.90 (d, J=8.0 Hz, 1H), 7.81 (s, 1H), 7.69 (d, J=8.0 Hz, 1H), 7.62 (t, J=7.9 Hz, 1H), 7.53 (s, 1H), 4.23 (s, 1H), 3.20 (d, J=4.9 Hz, 4H), 3.06 (s, 3H), 2.09-1.94 (m, 4H). LCMS (ESI) calcd. for C₂₀H₂₀ClF₅N₃O₂S [M+H]⁺ m/z 496.09, found 496.0.

### Example 223 and Example 224

### (S)-6-cyclopropyl-2-(4,4-difluoroazepan-1-yl)-4-methyl-N-(3-(S-methylsulfonimidoyl)phenyl)-5-(trifluoromethyl)nicotinamide and (R)-6-cyclopropyl-2-(4,4-difluoroazepan-1-yl)-4-methyl-N-(3-(S-methylsulfonimidoyl)phenyl)-5-(trifluoromethyl)nicotinamide

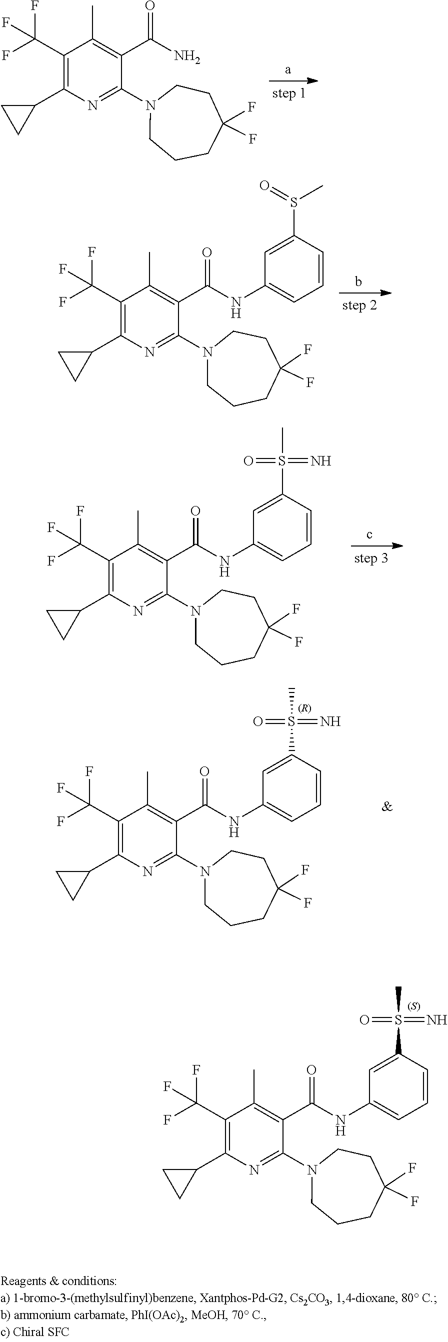

Step 1: 6-cyclopropyl-2-(4,4-difluoroazepan-1-yl)-4-methyl-N-(3-(methylsulfinyl)phenyl)-5-(trifluoromethyl)nicotinamide: A solution of 6-cyclopropyl-2-(4,4-difluoroazepan-1-yl)-4-methyl-5-(trifluoromethyl)nicotinamide (200 mg, 0.53 mmol), Cs₂CO₃ (522 mg, 1.59 mmol), Xantphos-Pd-G2 (96 mg, 0.11 mmol) and 1-bromo-3-(methylsulfinyl)benzene (140 mg, 0.64 mmol) in 1,4-dioxane (5 mL) was heated to 80° C. and stirred for 16 hours under N₂. After the reaction was completed, the resulting solution diluted with water (20 mL) and extracted with EtOAc (20 mL×3). The combined organic layers were washed with brine, dried over Na₂SO₄, concentrated under vacuum. The residue was purified by flash column chromatography on silica gel (PE/EtOAc=1/1 to 1/2) to give 6-cyclopropyl-2-(4,4-difluoroazepan-1-yl)-4-methyl-N-(3-(methylsulfinyl)phenyl)-5-(trifluoromethyl)nicotinamide (200 mg, 73.3%) as a yellow solid. LCMS (ESI) calcd. for C₂₄H₂₇F₅N₃O₂S [M+H]⁺ m/z 516.17, found 515.9.

Step 2: 6-cyclopropyl-2-(4,4-difluoroazepan-1-yl)-4-methyl-N-(3-(S-methylsulfonimidoyl)phenyl)-5-(trifluoromethyl)nicotinamide: A solution of 6-cyclopropyl-2-(4,4-difluoroazepan-1-yl)-4-methyl-N-(3-(methylsulfinyl)phenyl)-5-(trifluoromethyl)nicotinamide (200 mg, 0.39 mmol) in MeOH (10 mL) was added ammonium carbamate (73 mg, 1.17 mmol) and PhI(OAc)₂ (314 mg, 0.98 mmol) at room temperature. The reaction mixture was heated to 70° C. and stirred for 16 hours. After the reaction was completed, the mixture was concentrated under vacuum. The residue was purified by flash column chromatography on silica gel (PE/EtOAc=2/1 to 1/1) to give 6-cyclopropyl-2-(4,4-difluoroazepan-1-yl)-4-methyl-N-(3-(S-methylsulfonimidoyl)phenyl)-5-(trifluoromethyl)nicotinamide (160 mg, 77.4%) as a yellow solid. LCMS (ESI) calcd. for C₂₄H₂₈F₅N₄O₂S [M+H]⁺ m/z 531.18, found 531.2.

Step 3: (S)-6-cyclopropyl-2-(4,4-difluoroazepan-1-yl)-4-methyl-N-(3-(S-methylsulfonimidoyl)phenyl)-5-(trifluoromethyl)nicotinamide and (R)-6-cyclopropyl-2-(4,4-difluoroazepan-1-yl)-4-methyl-N-(3-(S-methylsulfonimidoyl)phenyl)-5-(trifluoromethyl)nicotinamide: 6-cyclopropyl-2-(4,4-difluoroazepan-1-yl)-4-methyl-N-(3-(S-methylsulfonimidoyl)phenyl)-5-(trifluoromethyl)nicotinamide was further purified by Chiral-Prep-SFC ((R,R)-Whelk-01, 20 mm*250 mm L, 5 m, eluting with 75% CO₀₂/MeOH containing 0.1% NH₃) to provide (S)-6-cyclopropyl-2-(4,4-difluoroazepan-1-yl)-4-methyl-N-(3-(S-methylsulfonimidoyl)phenyl)-5-(trifluoromethyl)nicotinamide (64.3 mg) as the first elution: H NMR (400 MHz, DMSO-d₆) δ 10.81 (s, 1H), 8.31 (s, 1H), 7.89 (d, J=7.8 Hz, 1H), 7.67 (d, J=7.8 Hz, 1H), 7.58 (t, J=7.9 Hz, 1H), 4.22 (s, 1H), 3.70-3.50 (m, 4H), 3.06 (s, 3H), 2.33-2.27 (m, 3H), 2.27-2.15 (m, 3H), 2.03-1.91 (m, 2H), 1.83-1.74 (m, 2H), 1.07 (s, 2H), 1.03-0.90 (m, 2H). LCMS (ESI) calcd. for C₂₄H₂₈F₅N₄O₂S [M+H]⁺ m/z 531.18, found 531.1 and (R)-6-cyclopropyl-2-(4,4-difluoroazepan-1-yl)-4-methyl-N-(3-(S-methylsulfonimidoyl)phenyl)-5-(trifluoromethyl)nicotinamide (62.3 mg) as the second elution. ¹H NMR (400 MHz, DMSO-d₆) δ 10.81 (s, 1H), 8.31 (s, 1H), 7.89 (d, J=8.1 Hz, 1H), 7.67 (d, J=8.0 Hz, 1H), 7.58 (t, J=7.9 Hz, 1H), 4.21 (s, 1H), 3.68-3.52 (m, 4H), 3.06 (s, 3H), 2.31-2.27 (m, 3H), 2.26-2.16 (m, 3 H), 2.03-1.89 (m, 2H), 1.83-1.75 (m, 2H), 1.07 (s, 2H), 1.01-0.92 (m, 2H). LCMS (ESI) calcd. for C₂₄H₂₈F₅N₄O₂S [M+H]⁺ m/z 531.18, found 531.1.

### Example 225

### (R)-3-(4,4-difluoroazepan-1-yl)-5-methyl-N-(3-(S-methylsulfonimidoyl)phenyl)-6-(p-tolyl)pyridazine-4-carboxamide

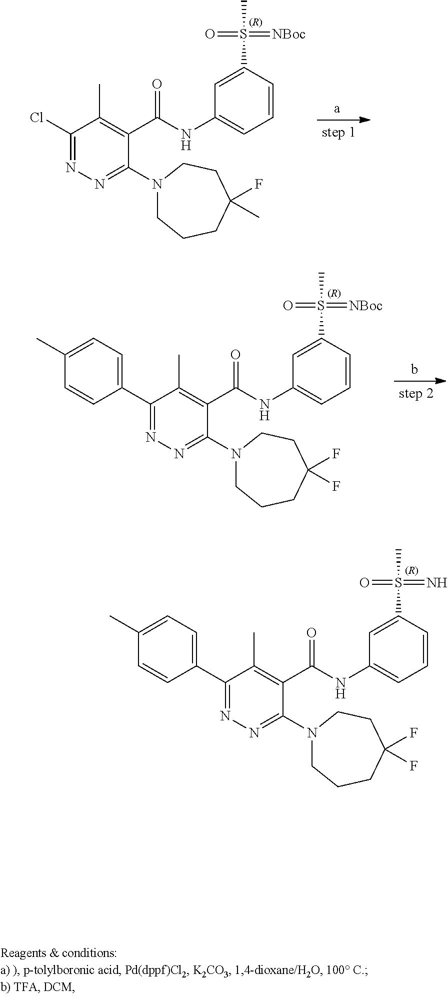

Step 1: tert-butyl (R)-((3-(3-(4,4-difluoroazepan-1-yl)-5-methyl-6-(p-tolyl)pyridazine-4-carboxamido)phenyl)(methyl)(oxo)-λ⁶-sulfaneylidene)carbamate: A mixture of tert-butyl (R)-((3-(6-chloro-3-(4,4-difluoroazepan-1-yl)-5-methylpyridazine-4-carboxamido)phenyl)(methyl)(oxo)-λ⁶-sulfaneylidene)carbamate (150 mg, 0.27 mmol), p-tolylboronic acid (74 mg, 0.54 mmol), K₂CO₃ (112 mg, 0.81 mmol) and Pd(dppf)Cl₂ (18 mg, 0.02 mmol) in 1,4-dioxane (8 mL) and H₂O (2 mL) was heated at 100° C. for 16 hours under an atmosphere of N₂. After the reaction was completed, the mixture was cooled to room temperature. The resulting solution was diluted with water (40 mL) and extracted with EtOAc (40 mL×3). The combined organic phases were washed with brine, dried over Na₂SO₄, concentrated under vacuum. The residue was purified by flash column chromatography on silica gel (PE/EtOAc=1/1) to give tert-butyl (R)-((3-(3-(4,4-difluoroazepan-1-yl)-5-methyl-6-(p-tolyl)pyridazine-4-carboxamido)phenyl)(methyl)(oxo)-λ⁶-sulfaneylidene)carbamate (105 mg, 63.6%) as a yellow solid. LCMS (ESI) calcd. for C₃₁H₃₈F₂N₅O₄S [M+H]⁺ m/z 614.26, found 614.20.

Step 2: (R)-3-(4,4-difluoroazepan-1-yl)-5-methyl-N-(3-(S-methylsulfonimidoyl)phenyl)-6-(p-tolyl)pyridazine-4-carboxamide: A solution of tert-butyl (R)-((3-(3-(4,4-difluoroazepan-1-yl)-5-methyl-6-(p-tolyl)pyridazine-4-carboxamido)phenyl)(methyl)(oxo)-λ⁶-sulfaneylidene)carbamate (105 mg, 0.17 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (30 mL×3). The combined organic phases were washed with brine, dried over sodium sulfate, concentrated under vacuum. The residue was purified by prep-HPLC (Gemini 5 um C₁₈ column, 150*21.2 mm, eluting with 35% to 90% MeCN/H₂O containing 0.1% NH₃) to provide (R)-3-(4,4-difluoroazepan-1-yl)-5-methyl-N-(3-(S-methylsulfonimidoyl)phenyl)-6-(p-tolyl)pyridazine-4-carboxamide (36.7 mg, 42.1%) as a white solid. ¹H NMR (400 MHz, DMSO-d₆) δ 11.07 (s, 1H), 8.32 (s, 1H), 7.89 (d, J=7.6 Hz, 1H), 7.70 (d, J=7.6 Hz, 1H), 7.61 (t, J=7.9 Hz, 1H), 7.45 (d, J=8.0 Hz, 2H), 7.32 (d, J=8.0 Hz, 2H), 4.25 (s, 1H), 3.84-3.70 (m, 2H), 3.65 (t, J=6.0 Hz, 2H), 3.06 (s, 3H), 2.44-2.25 (m, 5H), 2.19 (s, 3H), 2.14-2.00 (m, 2H), 1.92-1.80 (m, 2H). LCMS (ESI) calcd. for C₂₆H₃₀F₂N₅O₂S [M+H]⁺ m/z 514.21, found 514.15.

### Example 226

### (R)-5-chloro-2-(4,4-difluoroazepan-1-yl)-4-fluoro-N-(3-(S-methylsulfonimidoyl)phenyl)nicotinamide

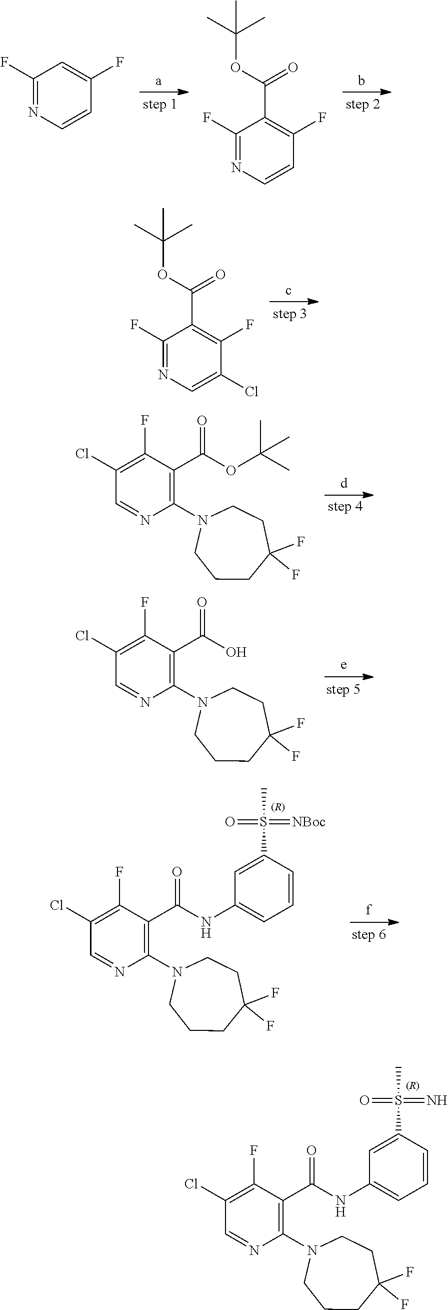

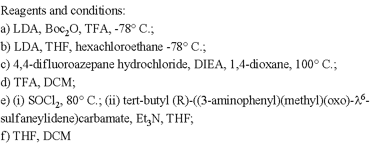

Step 1: tert-butyl 2,4-difluoronicotinate: To a solution of 2,4-difluoropyridine (5 g, 43.4 mmol) in THE (30 mL) at −78° C. under an atmosphere of N₂ was added a solution of LDA (32.2 mL, 2M in THF). The mixture was stirred at −78° C. for 10 min. Then a solution of Boc₂O (28.4 g, 130.2 mmol) in THE (20 mL) was added to the solution dropwise. The reaction mixture was stirred at 25° C. for 15 min. After the reaction was completed, the reaction was quenched with saturated NH₄Cl (100 mL) and extracted with EtOAc (150 mL×3). The combined organic layers were dried over sodium sulfate and evaporated to give the crude which was purified by flash column chromatography on silica gel (PE/EtAOc=10/1) to give tert-butyl 2,4-difluoronicotinate (7.3 g, 78.2%) as white oil. LCMS (ESI) calcd. for C₁₀H₁₂F₂NO₂ [M+H]⁺ m/z 216.09, found 215.90.

Step 2: tert-butyl 5-chloro-2,4-difluoronicotinate: To a solution of tert-butyl 2,4-difluoronicotinate (1.5 g, 7 mmol) in THE (15 mL) was added LDA (5.2 mL, 2M, in THF) at −78° C. under an atmosphere of N₂. The mixture was stirred at −78° C. for 30 min. Then a solution of hexachloroethane (2.07 g, 8.75 mmol) in THE (10 mL) was added to the mixture dropwise. The reaction mixture was stirred at 25° C. for 1 hour. After the reaction was completed, the solution was quenched with saturated NH₄Cl (80 mL) and extracted with EtOAc (100 mL×3). The combined organic layers were dried over sodium sulfate and evaporated to give the crude which was purified by flash column chromatography on silica gel (PE/EtAOc=10/1) to give tert-butyl 5-chloro-2,4-difluoronicotinate (600 mg, 34.4%) as white oil. LCMS (ESI) calcd. for C₁₀H₁₁ClF₂NO₂ [M+H]⁺ m/z 250.05, found 250.00.

Step 3: tert-butyl 5-chloro-2-(4,4-difluoroazepan-1-yl)-4-fluoronicotinate: A mixture of tert-butyl 5-chloro-2,4-difluoronicotinate (300 mg, 1.2 mmol), 4,4-difluoroazepane hydrochloride (247 mg, 1.44 mmol) and DIEA (464.5 mg, 3.6 mmol) in 1,4-dioxane (5 mL) was heated at 100° C. for 5 hours. After the reaction was completed, the mixture was cooled to room temperature. Then the mixture was concentrated to give the crude. The residue was purified by flash column chromatography on silica gel (PE/EtOAc=10/1) to give tert-butyl 5-chloro-2-(4,4-difluoroazepan-1-yl)-4-fluoronicotinate (400 mg, 91.6%). LCMS (ESI) calcd. for C₁₆H₂₁ClF₃N₂O₂ [M+H]⁺ m/z 365.13, found 365.10.

Step 4: 5-chloro-2-(4,4-difluoroazepan-1-yl)-4-fluoronicotinic acid: A solution of tert-butyl 5-chloro-2-(4,4-difluoroazepan-1-yl)-4-fluoronicotinate (400 mg, 1.1 mmol) in DCM (3 mL) was added TFA (3 mL) at room temperature. The reaction mixture was stirred at room temperature for 2 hours. After the reaction was completed, the mixture was concentrated to give 5-chloro-2-(4,4-difluoroazepan-1-yl)-4-fluoronicotinic acid (320 mg, 94.4%) as a white solid. LCMS (ESI) calcd. for C₁₂H₁₃ClF₃N₂O₂ [M+H]⁺ m/z 309.06, found 308.75.

Step 5: tert-butyl (R)-((3-(5-chloro-2-(4,4-difluoroazepan-1-yl)-4-fluoronicotinamido)phenyl)(methyl)(oxo)-λ⁶-sulfaneylidene)carbamate: A solution of 5-chloro-2-(4,4-difluoroazepan-1-yl)-4-fluoronicotinic acid (260 mg, 0.84 mmol) in SOCl₂ (5 mL) was heated at 80° C. for 0.5 h. The solution was concentrated under vacuum to provide the chloride intermediate. Then the chloride intermediate dissolved in THF (2 mL) was added to a solution of tert-butyl (R)-((3-aminophenyl)(methyl)(oxo)-λ⁶-sulfaneylidene)carbamate (274.3 mg, 1.01 mmol) and Et₃N (255.7 mg, 2.53 mmol) in THE (3 mL) at 0° C. The resulting mixture was stirred at 25° C. for 1 h. Then the mixture was quenched with water (50 mL) and extracted with DCM (50 mL×3). The combined organic phases were washed with brine, dried over sodium sulfate, concentrated under vacuum. The residue was purified by flash column chromatography on silica gel (PE/EtOAc=1/1) to provide tert-butyl (R)-((3-(5-chloro-2-(4,4-difluoroazepan-1-yl)-4-fluoronicotinamido)phenyl)(methyl)(oxo)-λ⁶-sulfaneylidene)carbamate (150 mg, 31.8%) as a white oil. LCMS (ESI) calcd. for C₂₄H₂₉ClF₃N₄O₄S [M+H]⁺ m/z 561.16, found 561.10.

Step 6: (R)-5-chloro-2-(4,4-difluoroazepan-1-yl)-4-fluoro-N-(3-(S-methylsulfonimidoyl)phenyl)nicotinamide: A solution of tert-butyl (R)-((3-(5-chloro-2-(4,4-difluoroazepan-1-yl)-4-fluoronicotinamido)phenyl)(methyl)(oxo)-λ⁶-sulfaneylidene)carbamate (150 mg, 0.27 mmol) in DCM (5 mL) was added TFA (1 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 40% to 90% MeCN/H₂O containing 0.05% NH₃) to provide (R)-5-chloro-2-(4,4-difluoroazepan-1-yl)-4-fluoro-N-(3-(S-methylsulfonimidoyl)phenyl)nicotinamide (40.5 mg, 32.6%) as a white solid. ¹H NMR (400 MHz, DMSO-d₆) δ 11.11 (s, 1H), 8.36 (d, J=10.1 Hz, 1H), 8.26 (d, J=1.9 Hz, 1H), 7.92-7.86 (m, 1H), 7.72-7.66 (m, 1H), 7.61 (s, 1H), 4.24 (s, 1H), 3.73-3.60 (m, 2 H), 3.53 (s, 2H), 3.07 (s, 3H), 2.35-2.26 (m, 2H), 2.06-1.93 (m, 2H), 1.88-1.79 (m, 2H). LCMS (ESI) calcd. for C₁₉H₂₁ClF₃N₄O₂S [M+H]⁺ m/z 461.10, found 461.05.

### Example 227

### (R)-N-(3-(N-(2-amino-2-methylpropanoyl)-S-methylsulfonimidoyl)phenyl)-5-cyano-2-(4,4-difluoroazepan-1-yl)-4-methyl-6-(trifluoromethyl)nicotinamide formate

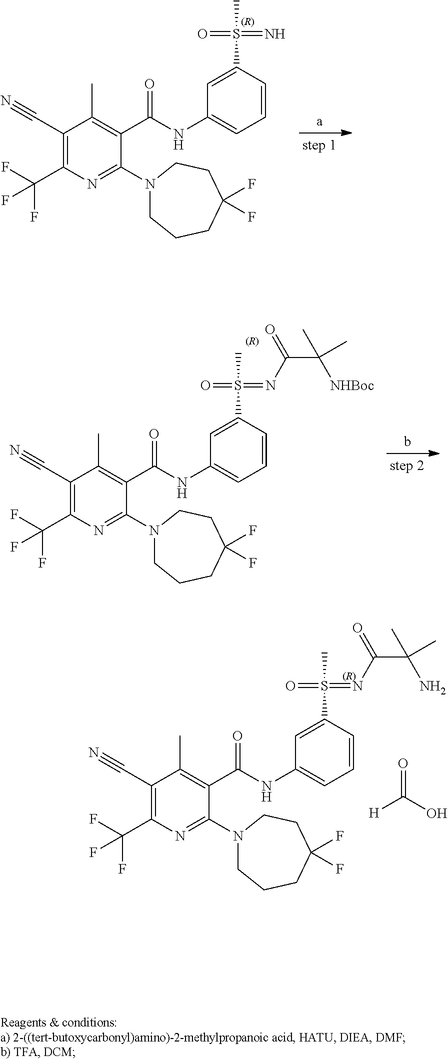

Step 1: tert-butyl (R)-(1-(((3-(5-cyano-2-(4,4-difluoroazepan-1-yl)-4-methyl-6-(trifluoromethyl)nicotinamido)phenyl)(methyl)(oxo)-λ⁶-sulfaneylidene)amino)-2-methyl-1-oxopropan-2-yl)carbamate: A solution of (R)-5-cyano-2-(4,4-difluoroazepan-1-yl)-4-methyl-N-(3-(S-methylsulfonimidoyl)phenyl)-6-(trifluoromethyl)nicotinamide (200 mg, 0.39 mmol), 2-((tert-butoxycarbonyl)amino)-2-methylpropanoic acid (95 mg, 0.47 mmol), HATU (221 mg, 0.58 mmol) and DIEA (150 mg, 1.16 mmol) in DMF (5 mL) was stirred at room temperature for 16 hours. LCMS showed the reaction was completed. The mixture was diluted with water (40 mL) and extracted with EtOAc (30 mL×3). The combined organic layers were washed with brine, dried over sodium sulfate, concentrated under vacuum. The residue was purified by flash column chromatography on silica gel (DCM/MeOH=95/5) to provide tert-butyl (R)-(1-(((3-(5-cyano-2-(4,4-difluoroazepan-1-yl)-4-methyl-6-(trifluoromethyl)nicotinamido)phenyl)(methyl)(oxo)-λ⁶-sulfaneylidene)amino)-2-methyl-1-oxopropan-2-yl)carbamate (75 mg, 27.6%) as brown oil. LCMS (ESI) calcd. for C₃₁H₃₈F₅N₆O₅S [M+H]⁺ m/z 701.26, found 701.25.

Step 2: (R)-N-(3-(N-(2-amino-2-methylpropanoyl)-S-methylsulfonimidoyl)phenyl)-5-cyano-2-(4,4-difluoroazepan-1-yl)-4-methyl-6-(trifluoromethyl)nicotinamide formate: A solution of tert-butyl (R)-(1-(((3-(5-cyano-2-(4,4-difluoroazepan-1-yl)-4-methyl-6-(trifluoromethyl)nicotinamido)phenyl)(methyl)(oxo)-λ⁶-sulfaneylidene)amino)-2-methyl-1-oxopropan-2-yl)carbamate (75 mg, 0.11 mmol) in DCM (4 mL) was added TFA (0.4 mL) at room temperature. The reaction mixture was stirred at room temperature for 1 hour. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 30% to 95% MeCN/H₂O containing 0.1% formic acid) to provide (R)-N-(3-(N-(2-amino-2-methylpropanoyl)-S-methylsulfonimidoyl)phenyl)-5-cyano-2-(4,4-difluoroazepan-1-yl)-4-methyl-6-(trifluoromethyl)nicotinamide formate (31.2 mg, 48.1%) as a white solid. ¹H NMR (400 MHz, DMSO-d₆) δ 11.16 (s, 1H), 8.43 (s, 1H), 8.11 (s, 3H), 7.90 (d, J=8.1 Hz, 1H), 7.79-7.68 (m, 2H), 3.88-3.61 (m, 4H), 3.59 (s, 3H), 2.47 (s, 3H), 2.38-2.23 (m, 2H), 2.11-1.98 (m, 2H), 1.91-1.79 (m, 2H), 1.49 (s, 3H), 1.45 (s, 3H). LCMS (ESI) calcd. for C₂₆H₃₀F₅N₆O₃S [M+H]⁺ m/z 601.20, found 601.15.

### Example 228

### (R)-5-chloro-2-(4,4-difluoroazepan-1-yl)-N-(3-(S-methyl-N-(methylglycyl)sulfonimidoyl)phenyl)-4-(trifluoromethyl)benzamide

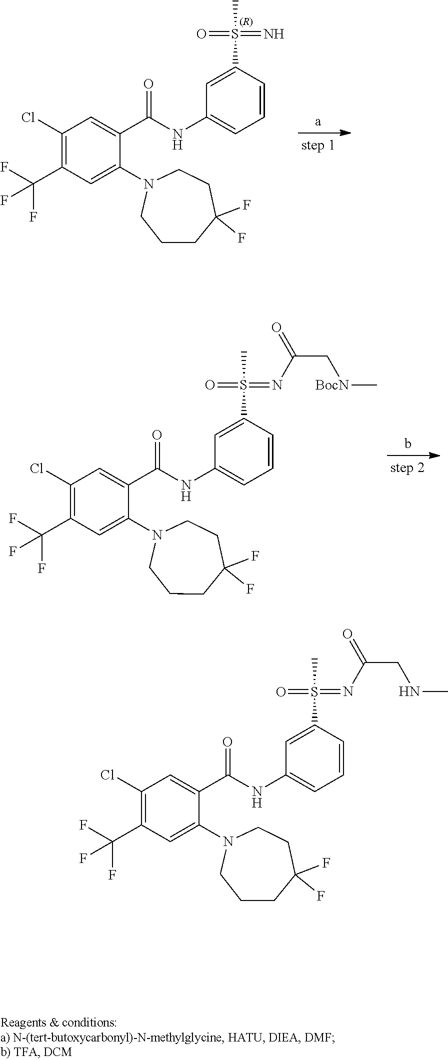

Step 1: tert-butyl (R)-(2-(((3-(5-chloro-2-(4,4-difluoroazepan-1-yl)-4-(trifluoromethyl)benzamido)phenyl)(methyl)(oxo)-λ⁶-sulfaneylidene)amino)-2-oxoethyl)(methyl)carbamate: A mixture of (R)-5-chloro-2-(4,4-difluoroazepan-1-yl)-N-(3-(S-methylsulfonimidoyl)phenyl)-4-(trifluoromethyl)benzamide (100 mg, 0.196 mmol) and N-(tert-butoxycarbonyl)-N-methylglycine (63 mg, 0.334 mmol) in DMF (5 mL) was added HATU (97 mg, 0.26 mmol) and DIEA (81 mg, 0.62 mmol). The mixture was stirred at room temperature for 4 hours. The reaction was monitored by LCMS. After the reaction was completed, the resulting solution was diluted with water (20 mL) and extracted with DCM (20 mL×3). The combined organic phases were washed with brine, dried over Na₂SO₄, concentrated under vacuum. The residue was purified by flash column chromatography on silica gel (PE/EtOAc=2/1 to 1/1) to give tert-butyl (R)-(2-(((3-(5-chloro-2-(4,4-difluoroazepan-1-yl)-4-(trifluoromethyl)benzamido)phenyl)(methyl)(oxo)-λ⁶-sulfaneylidene)amino)-2-oxoethyl)(methyl)carbamate (80 mg, 70.17%) as a white oil. LCMS (ESI) calcd. for C 29H₃₅ClF₅N₄O₅S [M+H]⁺ m/z 681.19, found 681.00.

Step 2: (R)-5-chloro-2-(4,4-difluoroazepan-1-yl)-N-(3-(S-methyl-N-(methylglycyl)sulfonimidoyl)phenyl)-4-(trifluoromethyl)benzamide: A solution of tert-butyl (R)-(2-(((3-(5-chloro-2-(4,4-difluoroazepan-1-yl)-4-(trifluoromethyl)benzamido)phenyl)(methyl)(oxo)-λ⁶--sulfaneylidene)amino)-2-oxoethyl)(methyl)carbamate (80 mg, 0.14 mmol) in DCM (5 mL) was added TFA (0.5 mL) at room temperature. The reaction mixture was stirred at room temperature for 2 hours. After the reaction was completed, the solution was diluted with water (10 mL) and extracted with DCM (10 mL×3). The combined organic phases were washed with brine, dried over sodium sulfate, concentrated under vacuum. The residue was purified by prep-HPLC (Gemini 5 um C₁₈ column, 150*21.2 mm, eluting with 40% to 90% MeCN/H₂O containing 0.1% formic acid) to obtain (R)-5-chloro-2-(4,4-difluoroazepan-1-yl)-N-(3-(S-methyl-N-(methylglycyl)sulfonimidoyl)phenyl)-4-(trifluoromethyl)benzamide (37.5 mg, 46.25%) as a white solid. ¹H NMR (400 MHz, DMSO-d₆, ppm) δ 10.97 (s, 1H), 8.36 (s, 1H), 7.99-7.97 (m, 1H), 7.72-7.65 (m, 3H), 7.34 (s, 1H), 3.46 (s, 3H), 3.43-3.40 (m, 2H), 3.39-3.36 (m, 2H), 3.20 (s, 2H), 2.20-2.25 (m, 5H), 2.08-2.05 (m, 2H), 1.82-1.80 (m, 2H). LCMS (ESI) calcd. for C₂₄H₂₇ClF₅N₄O₃S [M+H]⁺ m/z 581.14, found 581.10.

### Example 229

### (R)-2-(4,4-difluoroazepan-1-yl)-N-(3-(N-(2-hydroxyacetyl)-S-methylsulfonimidoyl)phenyl)-4-methyl-5-(1-methyl-1H-pyrazol-4-yl)nicotinamide

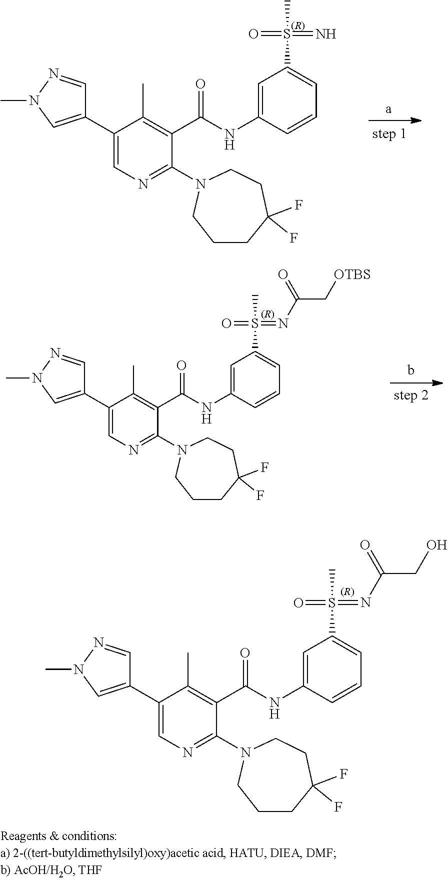

Step 1: (R)-N-(3-(N-(2-((tert-butyldimethylsilyl)oxy)acetyl)-S-methylsulfonimidoyl)phenyl)-2-(4,4-difluoroazepan-1-yl)-4-methyl-5-(1-methyl-1H-pyrazol-4-yl)nicotinamide: To a solution of (R)-2-(4,4-difluoroazepan-1-yl)-4-methyl-5-(1-methyl-1H-pyrazol-4-yl)-N-(3-(S-methylsulfonimidoyl)phenyl)nicotinamide (200 mg, 0.40 mmol), 2-((tert-butyldimethylsilyl)oxy)acetic acid (378 mg, 1.99 mmol) and HATU (303 mg, 0.80 mmol) in DMF (5 mL) and was added DIEA (154 mg, 1.20 mmol). Then the mixture was heated at 25° C. for 2 hours. LCMS showed the reaction was completed. The mixture was diluted with water and extracted with EtOAc (3×30 mL). The combined organic layers were washed with brine, dried with Na₂SO₄, and concentrated under vacuum. The residue was purified by flash column chromatography on silica gel (PE/EtOAc=1/1) to give (R)-N-(3-(N-(2-((tert-butyldimethylsilyl)oxy)acetyl)-S-methylsulfonimidoyl)phenyl)-2-(4,4-difluoroazepan-1-yl)-4-methyl-5-(1-methyl-1H-pyrazol-4-yl)nicotinamide (110 mg, 38.46%) as a yellow oil. LCMS (ESI) calcd. for C₃₂H₄₅F₂N₆O₄SSi [M+H]⁺ m/z 675.30, found 675.25.

Step 2: (R)-2-(4,4-difluoroazepan-1-yl)-N-(3-(N-(2-hydroxyacetyl)-S-methylsulfonimidoyl)phenyl)-4-methyl-5-(1-methyl-1H-pyrazol-4-yl)nicotinamide: To a solution of (R)-N-(3-(N-(2-((tert-butyldimethylsilyl)oxy)acetyl)-S-methylsulfonimidoyl)phenyl)-2-(4,4-difluoroazepan-1-yl)-4-methyl-5-(1-methyl-1H-pyrazol-4-yl)nicotinamide (110 mg, 0.16 mmol) in THE (1 mL) was added H₂O (3 mL) and AcOH (3 mL) at room temperature. The reaction mixture was stirred at room temperature for 2 hours. After the reaction was completed, the mixture was diluted with water and extracted with EtOAc (3×30 mL). The combined organic layers were washed with brine, dried with Na₂SO₄, and concentrated under vacuum. The residue was purified by prep-HPLC (Gemini 5 um C₁₈ column, 150*19 mm, eluting with 20% to 30% ACN/H₂O containing 0.1% formic acid) to provide (R)-2-(4,4-difluoroazepan-1-yl)-N-(3-(N-(2-hydroxyacetyl)-S-methylsulfonimidoyl)phenyl)-4-methyl-5-(1-methyl-1H-pyrazol-4-yl)nicotinamide (45 mg, 49.45%) as a white solid. ¹H NMR (400 MHz, DMSO-d₆) δ 10.94 (s, 1H), 8.35 (s, 1H), 8.18 (s, 1H), 8.04-7.96 (m, 1H), 7.90 (s, 1H), 7.70-7.63 (m, 2H), 7.61 (s, 1H), 4.78 (t, J=6.3 Hz, 1H), 3.95 (d, J=6.3 Hz, 2H), 3.88 (s, 3H), 3.61 (dd, J=6.5, 3.8 Hz, 2H), 3.54 (t, J=6.1 Hz, 2H), 3.46 (s, 3H), 2.34-2.20 (m, 5H), 2.07-1.93 (m, 2H), 1.86-1.75 (m, 2H). LCMS (ESI) calcd. for C₂₆H₃₁F₂N₆O₄S [M+H]⁺ m/z 561.21, found 561.05.

### Example 230

### 2-(4,4-difluoroazepan-1-yl)-N-(3-((R)-N-((R)-2-hydroxypropanoyl)-S-methylsulfonimidoyl)phenyl)-4-methyl-5-(1-methyl-1H-pyrazol-4-yl)nicotinamide

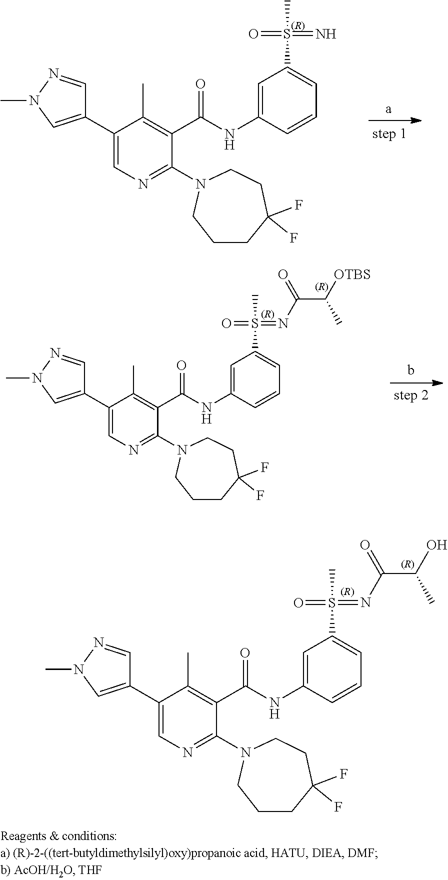

Step 1: N-(3-((R)-N-((R)-2-((tert-butyldimethylsilyl)oxy)propanoyl)-S-methylsulfonimidoyl)phenyl)-2-(4,4-difluoroazepan-1-yl)-4-methyl-5-(1-methyl-1H-pyrazol-4-yl)nicotinamide: To a solution of (R)-2-(4,4-difluoroazepan-1-yl)-4-methyl-5-(1-methyl-1H-pyrazol-4-yl)-N-(3-(S-methylsulfonimidoyl)phenyl)nicotinamide (200 mg, 0.40 mmol), (R)-2-((tert-butyldimethylsilyl)oxy)propanoic acid (406 mg, 1.99 mmol) and HATU (303 mg, 0.80 mmol) in DMF (5 mL) was added DIEA (154 mg, 1.20 mmol). Then the mixture was heated at 25° C. for 2 hours. LCMS showed the reaction was completed. The mixture was diluted with water and extracted with EtOAc (3×30 mL). The combined organic layers were washed with brine, dried with Na₂SO₄, and concentrated under vacuum. The residue was purified by flash column chromatography on silica gel (PE/EtOAc=1/1) to give N-(3-((R)-N-((R)-2-((tert-butyldimethylsilyl)oxy)propanoyl)-S-methylsulfonimidoyl)phenyl)-2-(4,4-difluoroazepan-1-yl)-4-methyl-5-(1-methyl-1H-pyrazol-4-yl)nicotinamide (110 mg, 40.15%) as a yellow oil. LCMS (ESI) calcd. for C₃₃H₄₇F₂N₆O₄SSi [M+H]⁺ m/z 689.31, found 689.25

Step 2: 2-(4,4-difluoroazepan-1-yl)-N-(3-((R)-N-((R)-2-hydroxypropanoyl)-S-methylsulfonimidoyl)phenyl)-4-methyl-5-(1-methyl-1H-pyrazol-4-yl)nicotinamide: To a solution of N-(3-((R)-N-((R)-2-((tert-butyldimethylsilyl)oxy)propanoyl)-S-methylsulfonimidoyl)phenyl)-2-(4,4-difluoroazepan-1-yl)-4-methyl-5-(1-methyl-1H-pyrazol-4-yl)nicotinamide (110 mg, 0.16 mmol) in THE (1 mL) was added H₂O (3 mL) and AcOH (3 mL) at room temperature. The reaction mixture was stirred at room temperature for 2 hours. After the reaction was completed, the mixture was diluted with water and extracted with EtOAc (3×30 mL). The combined organic layers were washed with brine, dried with Na₂SO₄, and concentrated under vacuum. The residue was purified by prep-HPLC (Gemini 5 um C₁₈ column, 150*19 mm, eluting with 20% to 30% ACN/H₂O containing 0.1% formic acid) to provide 2-(4,4-difluoroazepan-1-yl)-N-(3-((R)-N-((R)-2-hydroxypropanoyl)-S-methylsulfonimidoyl)phenyl)-4-methyl-5-(1-methyl-1H-pyrazol-4-yl)nicotinamide (47 mg, 51.25%) as a white solid. ¹H NMR (400 MHz, DMSO-d₆) δ 10.94 (s, 1H), 8.39 (s, 1H), 8.18 (s, 1H), 8.01-7.95 (m, 1H), 7.90 (s, 1H), 7.70-7.63 (m, 2H), 7.61 (s, 1H), 4.71 (d, J=5.3 Hz, 1H), 4.08-3.99 (m, 1H), 3.88 (s, 3H), 3.65-3.57 (m, 2H), 3.54 (t, J=6.1 Hz, 2H), 3.47 (s, 3H), 2.34-2.18 (m, 5H), 2.07-1.93 (m, 2H), 1.86-1.73 (m, 2H), 1.27 (d, J=6.8 Hz, 3H). LCMS (ESI) calcd. for C₂₇H₃₃F₂N₆O₄S [M+H]⁺ m/z 575.23, found 575.15.

### Example 231

### 2-(4,4-difluoroazepan-1-yl)-N-(3-((R)-N-((S)-2-hydroxypropanoyl)-S-methylsulfonimidoyl)phenyl)-4-methyl-5-(1-methyl-1H-pyrazol-4-yl)nicotinamide

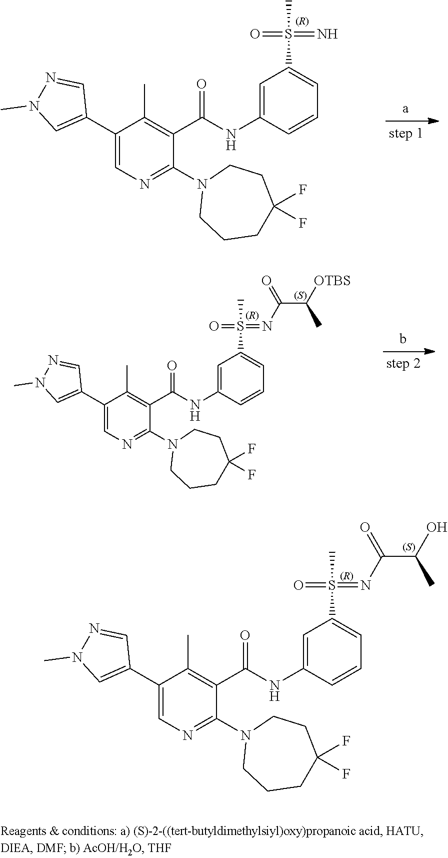

Step 1: N-(3-((R)-N-((S)-2-((tert-butyldimethylsilyl)oxy)propanoyl)-S-methylsulfonimidoyl)phenyl)-2-(4,4-difluoroazepan-1-yl)-4-methyl-5-(1-methyl-1H-pyrazol-4-yl)nicotinamide: To a solution of (R)-2-(4,4-difluoroazepan-1-yl)-4-methyl-5-(1-methyl-1H-pyrazol-4-yl)-N-(3-(S-methylsulfonimidoyl)phenyl)nicotinamide (200 mg, 0.40 mmol), (S)-2-((tert-butyldimethylsilyl)oxy)propanoic acid (406 mg, 1.99 mmol) and HATU (303 mg, 0.80 mmol) in DMF (5 mL) was added DIEA (154 mg, 1.20 mmol). Then the mixture was heated at 25° C. for 2 hours. LCMS showed the reaction was completed. The mixture was diluted with water and extracted with EtOAc (3×30 mL). The combined organic layers were washed with brine, dried with Na₂SO₄, and concentrated under vacuum. The residue was purified by flash column chromatography on silica gel (PE/EtOAc=1/1) to give N-(3-((R)-N-((S)-2-((tert-butyldimethylsilyl)oxy)propanoyl)-S-methylsulfonimidoyl)phenyl)-2-(4,4--1-yl)-4-methyl-5-(1-methyl-1H-pyrazol-4-yl)nicotinamide (120 mg, 43.8%) as a yellow oil. LCMS (ESI) calcd. for C₃₃H₄₇F₂N₆O₄SSi [M+H]⁺ m/z 689.31, found 689.25.

Step 2: 2-(4,4-difluoroazepan-1-yl)-N-(3-((R)-N-((S)-2-hydroxypropanoyl)-S-methylsulfonimidoyl)phenyl)-4-methyl-5-(1-methyl-1H-pyrazol-4-yl)nicotinamide: To a solution of N-(3-((R)-N-((S)-2-((tert-butyldimethylsilyl)oxy)propanoyl)-S-methylsulfonimidoyl)phenyl)-2-(4,4-difluoroazepan-1-yl)-4-methyl-5-(1-methyl-1H-pyrazol-4-yl)nicotinamide (120 mg, 0.17 mmol) in THE (1 mL) was added H₂O (3 mL) and AcOH (3 mL) at room temperature. The reaction mixture was stirred at room temperature for 2 hours. After the reaction was completed, the mixture was diluted with water and extracted with EtOAc (3×30 mL). The combined organic layers were washed with brine, dried with Na₂SO₄, and concentrated under vacuum. The residue was purified by prep-HPLC (Gemini 5 um C₁₈ column, 150*19 mm, eluting with 20% to 30% ACN/H₂O containing 0.1% formic acid) to provide 2-(4,4-difluoroazepan-1-yl)-N-(3-((R)-N-((S)-2-hydroxypropanoyl)-S-methylsulfonimidoyl)phenyl)-4-methyl-5-(1-methyl-1H-pyrazol-4-yl)nicotinamide (72 mg, 71.86%) as a white solid. ¹H NMR (400 MHz, DMSO-d₆, ppm) δ 10.94 (s, 1H), 8.41 (s, 1H), 8.18 (s, 1H), 7.99-7.93 (m, 1H), 7.90 (s, 1H), 7.70-7.63 (m, 2H), 7.61 (d, J=0.6 Hz, 1H), 4.69 (d, J=5.5 Hz, 1H), 4.11-3.98 (m, 1H), 3.88 (s, 3H), 3.65-3.58 (m, 2H), 3.54 (t, J=6.0 Hz, 2H), 3.45 (s, 3H), 2.35-2.20 (m, 5H), 2.06-1.91 (m, 2H), 1.87-1.74 (m, 2H), 1.26 (d, J=6.8 Hz, 3H). LCMS (ESI) calcd. for C₂₇H₃₃F₂N₆O₄S [M+H]⁺ m/z 575.23, found 575.05.

### Example 232

### (R)-3-(4,4-difluoroazepan-1-yl)-N-(3-(N-(2-hydroxyacetyl)-S-methylsulfonimidoyl)phenyl)-5-methyl-6-(trifluoromethyl)pyridazine-4-carboxamide

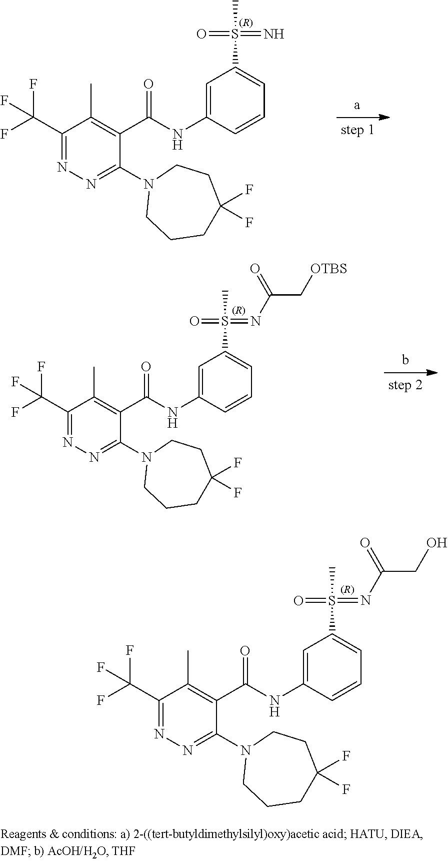

Step 1: (R)-N-(3-(N-(2-((tert-butyldimethylsilyl)oxy)acetyl)-S-methylsulfonimidoyl)phenyl)-3-(4,4-difluoroazepan-1-yl)-5-methyl-6-(trifluoromethyl)pyridazine-4-carboxamide: A mixture of (R)-3-(4,4-difluoroazepan-1-yl)-5-methyl-N-(3-(S-methylsulfonimidoyl)phenyl)-6-(trifluoromethyl)pyridazine-4-carboxamide (100 mg, 0.20 mmol) and 2-((tert-butyldimethylsilyl)oxy)acetic acid (77 mg, 0.40 mmol) in DMF (5 mL) was added HATU (114 mg, 0.30 mmol) and DIEA (77 mg, 0.60 mmol). The mixture was stirred at room temperature for 16 hours. The reaction was monitored by LCMS. After the reaction was completed, the resulting solution was diluted with water (50 mL) and extracted with DCM (20 mL×3). The combined organic phases were washed with brine, dried over Na₂SO₄, concentrated under vacuum. The residue was purified by flash column chromatography on silica gel (PE/EtOAc=2/1 to 1/1) to give (R)-N-(3-(N-(2-((tert-butyldimethylsilyl)oxy)acetyl)-S-methylsulfonimidoyl)phenyl)-3-(4,4-difluoroazepan-1-yl)-5-methyl-6-(trifluoromethyl)pyridazine-4-carboxamide (80 mg, 60.2%) as a yellow solid. LCMS (ESI) calcd. for C₂₈H₃₉F₅N₅O₄SSi [M+H]⁺ m/z 664.24, found 664.20.

Step 2: (R)-3-(4,4-difluoroazepan-1-yl)-N-(3-(N-(2-hydroxyacetyl)-S-methylsulfonimidoyl)phenyl)-5-methyl-6-(trifluoromethyl)pyridazine-4-carboxamide: A solution of (R)-N-(3-(N-(2-((tert-butyldimethylsilyl)oxy)acetyl)-S-methylsulfonimidoyl)phenyl)-3-(4,4-difluoroazepan-1-yl)-5-methyl-6-(trifluoromethyl)pyridazine-4-carboxamide (80 mg, 0.12 mmol) in THE (1 mL) and H₂O (3 mL) was added AcOH (3 mL) at room temperature. The reaction mixture was stirred at room temperature for 2 hours. After the reaction was completed, the solution was diluted with water (30 mL) and extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 80% MeCN/H₂O containing 0.05% NH₃) to obtain (R)-3-(4,4-difluoroazepan-1-yl)-N-(3-(N-(2-hydroxyacetyl)-S-methylsulfonimidoyl)phenyl)-5-methyl-6-(trifluoromethyl)pyridazine-4-carboxamide(20.5 mg, 31%) as a white solid. ¹H NMR (400 MHz, DMSO-d₆) δ 11.21 (s, 1H), 8.26 (s, 1H), 7.98 (d, J=7.7 Hz, 1H), 7.77-7.66 (m, 2H), 4.81 (t, J=6.3 Hz, 1H), 3.95 (d, J=6.2 Hz, 2H), 3.91-3.63 (m, 4H), 3.48 (s, 3H), 2.46-2.28 (m, 5H), 2.12-2.01 (m, 2H), 1.95-1.83 (m, 2H). LCMS (ESI) calcd. for C₂₂H₂₅F₅N₅O₄S [M+H]⁺ m/z 550.16, found 549.95.

### Example 233

### 3-(4,4-difluoroazepan-1-yl)-N-(3-((R)-N-((S)-2-hydroxypropanoyl)-S-methylsulfonimidoyl)phenyl)-5-methyl-6-(trifluoromethyl)pyridazine-4-carboxamide

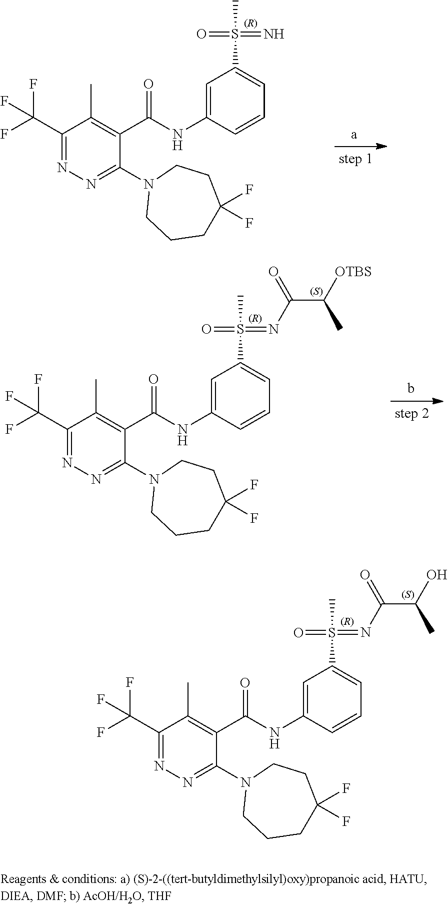

Step 1: N-(3-((R)-N-((S)-2-((tert-butyldimethylsilyl)oxy)propanoyl)-S-methylsulfonimidoyl)phenyl)-3-(4,4-difluoroazepan-1-yl)-5-methyl-6-(trifluoromethyl)pyridazine-4-carboxamide: A mixture of (R)-3-(4,4-difluoroazepan-1-yl)-5-methyl-N-(3-(S-methylsulfonimidoyl)phenyl)-6-(trifluoromethyl)pyridazine-4-carboxamide (100 mg, 0.20 mmol) and (S)-2-((tert-butyldimethylsilyl)oxy)propanoic acid (82 mg, 0.40 mmol) in DMF (5 mL) was added HATU (114 mg, 0.30 mmol) and DIEA (77 mg, 0.60 mmol). The mixture was stirred at room temperature for 16 hours. The reaction was monitored by LCMS. After the reaction was completed, the resulting solution was diluted with water (40 mL) and extracted with DCM (20 mL×3). The combined organic phases were washed with brine, dried over Na₂SO₄, concentrated under vacuum. The residue was purified by flash column chromatography on silica gel (PE/EtOAc=2/1 to 1/1) to give N-(3-((R)-N-((S)-2-((tert-butyldimethylsilyl)oxy)propanoyl)-S-methylsulfonimidoyl)phenyl)-3-(4,4-difluoroazepan-1-yl)-5-methyl-6-(trifluoromethyl)pyridazine-4-carboxamide (80 mg, 58.9%) as a yellow solid. LCMS (ESI) calcd. for C₂₉H₄₁F₅N₅O₄SSi [M+H]⁺ m/z 678.25, found 678.20.

Step 2: 3-(4,4-difluoroazepan-1-yl)-N-(3-((R)-N-((S)-2-hydroxypropanoyl)-S-methylsulfonimidoyl)phenyl)-5-methyl-6-(trifluoromethyl)pyridazine-4-carboxamide: A solution of N-(3-((R)-N-((S)-2-((tert-butyldimethylsilyl)oxy)propanoyl)-S-methylsulfonimidoyl)phenyl)-3-(4,4-difluoroazepan-1-yl)-5-methyl-6-(trifluoromethyl)pyridazine-4-carboxamide (80 mg, 0.12 mmol) in THE (1 mL) and H₂O (3 mL) was added AcOH (3 mL) at room temperature. The reaction mixture was stirred at room temperature for 2 hours. After the reaction was completed, the solution was diluted with water (10 mL) and extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 80% MeCN/H₂O containing 0.1% formic acid) to obtain 3-(4,4-difluoroazepan-1-yl)-N-(3-((R)-N-((S)-2-hydroxypropanoyl)-S-methylsulfonimidoyl)phenyl)-5-methyl-6-(trifluoromethyl)pyridazine-4-carboxamide (21.5 mg, 31.7%) as a white solid. ¹H NMR (400 MHz, DMSO-d₆) δ 11.21 (s, 1H), 8.31 (s, 1H), 7.98-7.86 (m, 1H), 7.79-7.63 (m, 2H), 4.72 (d, J=5.5 Hz, 1H), 4.04 (dd, J=6.8, 5.7 Hz, 1H), 3.94-3.61 (m, 4H), 3.46 (s, 3H), 2.43-2.29 (m, 5H), 2.13-2.00 (m, 2H), 1.94-1.82 (m, 2H), 1.25 (d, J=6.8 Hz, 3H). LCMS (ESI) calcd. for C₂₃H₂₇F₅N₅O₄S [M+H]⁺ m/z 564.17, found 564.00.

### Example 234

### (R)-N-(3-(N-(1-aminocyclobutane-1-carbonyl)-S-methylsulfonimidoyl)phenyl)-3-(4,4-difluoroazepan-1-yl)-5-methyl-6-(trifluoromethyl)pyridazine-4-carboxamide

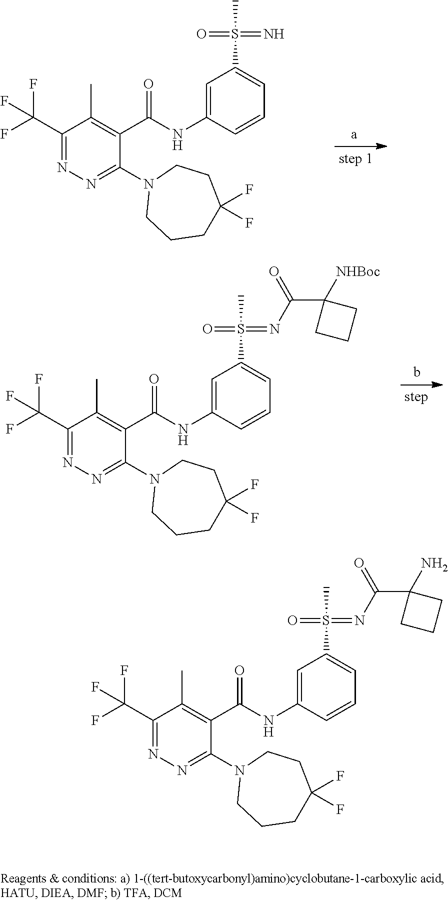 [SMILES: Cc1c(C(F)(F)F)nnc(N2CCCC(F)(F)CC2)c1C(=O)Nc1cccc([S@](C)(=N)=O)c1 ; Cc1c(C(F)(F)F)nnc(N2CCCC(F)(F)CC2)c1C(=O)Nc1cccc([S@@](C)(=O)=NC(=O)C2(NC(=O)OC(C)(C)C)CCC2)c1 ; Cc1c(C(F)(F)F)nnc(N2CCCC(F)(F)CC2)c1C(=O)Nc1cccc([S@@](C)(=O)=NC(=O)C2(N)CCC2)c1]

Step 1: tert-butyl (R)-(1-(((3-(3-(4,4-difluoroazepan-1-yl)-5-methyl-6-(trifluoromethyl)pyridazine-4-carboxamido)phenyl)(methyl)(oxo)-_⁶-sulfaneylidene)carbamoyl)cyclobutyl)carbamate: A mixture of (R)-3-(4,4-difluoroazepan-1-yl)-5-methyl-N-(3-(S-methylsulfonimidoyl)phenyl)-6-(trifluoromethyl)pyridazine-4-carboxamide (80 mg, 0.16 mmol) and 1-((tert-butoxycarbonyl)amino)cyclobutane-1-carboxylic acid (69 mg, 0.32 mmol) in DMF (5 mL) was added HATU (91 mg, 0.24 mmol) and DIEA (62 mg, 0.48 mmol). The mixture was heated at 50° C. for 16 hours. The reaction was monitored by LCMS. After the reaction was completed, the resulting solution was diluted with water (40 mL) and extracted with DCM (20 mL×3). The combined organic phases were washed with brine, dried over Na₂SO₄, concentrated under vacuum. The residue was purified by flash column chromatography on silica gel (PE/EtOAc=2/1 to 1/1) to give tert-butyl (R)-(1-(((3-(3-(4,4-difluoroazepan-1-yl)-5-methyl-6-(trifluoromethyl)pyridazine-4-carboxamido)phenyl)(methyl)(oxo)-λ⁶-sulfaneylidene)carbamoyl)cyclobutyl)carbamate (80 mg, 72.5%) as a yellow solid. LCMS (ESI) calcd. for C₃₀H₃₈F₅N₆O₅S [M+H]⁺ m/z 689.26, found 689.10.

Step 2: (R)-N-(3-(N-(1-aminocyclobutane-1-carbonyl)-S-methylsulfonimidoyl)phenyl)-3-(4,4-difluoroazepan-1-yl)-5-methyl-6-(trifluoromethyl)pyridazine-4-carboxamide: A solution of tert-butyl (R)-(1-(((3-(3-(4,4-difluoroazepan-1-yl)-5-methyl-6-(trifluoromethyl)pyridazine-4-carboxamido)phenyl)(methyl)(oxo)-λ⁶-sulfaneylidene)carbamoyl)cyclobutyl)carbamate (80 mg, 0.12 mmol) in DCM (5 mL) was added TFA (0.5 mL) at room temperature. The reaction mixture was stirred at room temperature for 2 hours. After the reaction was completed, the solution was diluted with water (10 mL) and extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 95% MeCN/H₂O containing 0.05% NH₃) to obtain (R)-N-(3-(N-(1-aminocyclobutane-1-carbonyl)-S-methylsulfonimidoyl)phenyl)-3-(4,4-difluoroazepan-1-yl)-5-methyl-6-(trifluoromethyl)pyridazine-4-carboxamide (23.6 mg, 33.3%) as a white solid. ¹H NMR (400 MHz, DMSO-d₆) δ 11.21 (s, 1H), 8.34 (s, 1H), 7.89 (s, 1H), 7.80-7.61 (m, 2H), 3.91-3.79 (m, 2H), 3.74-3.64 (m, 2H), 3.47 (s, 3H), 2.38-2.30 (m, 5H), 2.12-1.97 (m, 4H), 1.95-1.60 (m, 8H). LCMS (ESI) calcd. for C₂₅H₃₀F₅N₆O₃S [M+H]⁺ m/z 589.20, found 589.20.

### Example 235

### (R)-N-(3-(N-(2-amino-2-methylpropanoyl)-S-methylsulfonimidoyl)phenyl)-3-(4,4-difluoroazepan-1-yl)-5-methyl-6-(trifluoromethyl)pyridazine-4-carboxamide

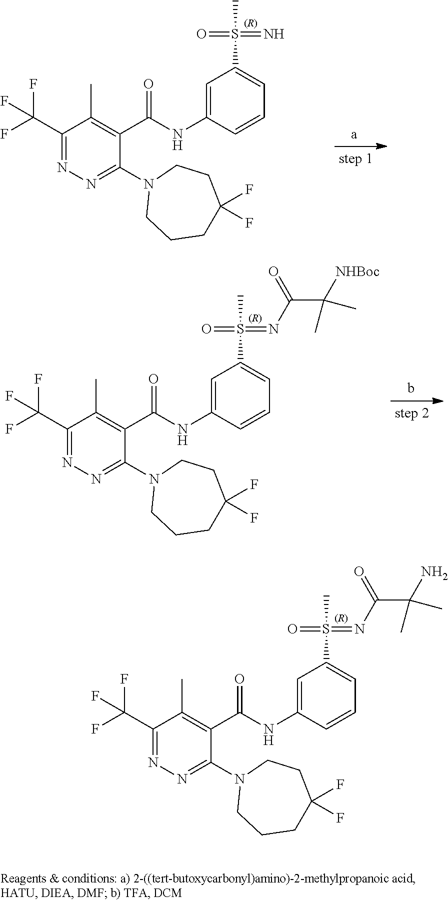

Step 1: tert-butyl (R)-(1-(((3-(3-(4,4-difluoroazepan-1-yl)-5-methyl-6-(trifluoromethyl)pyridazine-4-carboxamido)phenyl)(methyl)(oxo)-λ⁶-sulfaneylidene)amino)-2-methyl-1-oxopropan-2-yl)carbamate: A mixture of (R)-3-(4,4-difluoroazepan-1-yl)-5-methyl-N-(3-(S-methylsulfonimidoyl)phenyl)-6-(trifluoromethyl)pyridazine-4-carboxamide (80 mg, 0.16 mmol) and 2-((tert-butoxycarbonyl)amino)-2-methylpropanoic acid (65 mg, 0.32 mmol) in DMF (5 mL) was added HATU (91 mg, 0.24 mmol) and DIEA (62 mg, 0.48 mmol). The mixture was heated at 50° C. for 16 hours. The reaction was monitored by LCMS. After the reaction was completed, the resulting solution was diluted with water (20 mL) and extracted with DCM (20 mL×3). The combined organic phases were washed with brine, dried over Na₂SO₄, concentrated under vacuum. The residue was purified by flash column chromatography on silica gel (PE/EtOAc=2/1 to 1/1) to give tert-butyl (R)-(1-(((3-(3-(4,4-difluoroazepan-1-yl)-5-methyl-6-(trifluoromethyl)pyridazine-4-carboxamido)phenyl)(methyl)(oxo)-λ⁶-sulfaneylidene)amino)-2-methyl-1-oxopropan-2-yl)carbamate (60 mg, 55.3%) as a yellow solid. LCMS (ESI) calcd. for C₂₉H₃₈F₅N₆O₅S [M+H]⁺ m/z 677.25, found 677.30.

Step 2: (R)-N-(3-(N-(2-amino-2-methylpropanoyl)-S-methylsulfonimidoyl)phenyl)-3-(4,4-difluoroazepan-1-yl)-5-methyl-6-(trifluoromethyl)pyridazine-4-carboxamide: A solution of tert-butyl (R)-(1-(((3-(3-(4,4-difluoroazepan-1-yl)-5-methyl-6-(trifluoromethyl)pyridazine-4-carboxamido)phenyl)(methyl)(oxo)-λ⁶-sulfaneylidene)amino)-2-methyl-1-oxopropan-2-yl)carbamate (60 mg, 0.09 mmol) in DCM (5 mL) was added TFA (0.5 mL) at room temperature. The reaction mixture was stirred at room temperature for 2 hours. After the reaction was completed, the solution was diluted with water (10 mL) and extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 95% MeCN/H₂O containing 0.05% NH₃) to obtain (R)-N-(3-(N-(2-amino-2-methylpropanoyl)-S-methylsulfonimidoyl)phenyl)-3-(4,4-difluoroazepan-1-yl)-5-methyl-6-(trifluoromethyl)pyridazine-4-carboxamide (11.3 mg, 24.4%) as a white solid. ¹H NMR (400 MHz, DMSO-d₆) δ 11.20 (s, 1H), 8.32 (s, 1H), 7.90 (d, J=7.6 Hz, 1H), 7.76-7.63 (m, 2H), 3.95-3.64 (m, 4H), 3.44 (s, 3H), 2.39-2.30 (m, 5H), 2.14-2.01 (m, 2H), 1.92-1.83 (m, 2H), 1.75 (s, 2H), 1.20 (d, J=4.1 Hz, 6H). LCMS (ESI) calcd. for C₂₄H₃₀F₅N₆O₃S [M+H]⁺ m/z 577.20, found 577.15.

### Example 236

### 2-(4,4-difluoroazepan-1-yl)-N-(3-((R)-N-((R)-2-hydroxypropanoyl)-S-methylsulfonimidoyl)phenyl)-4-methyl-5-(1-methyl-1H-pyrazol-4-yl)-6-(trifluoromethyl)nicotinamide

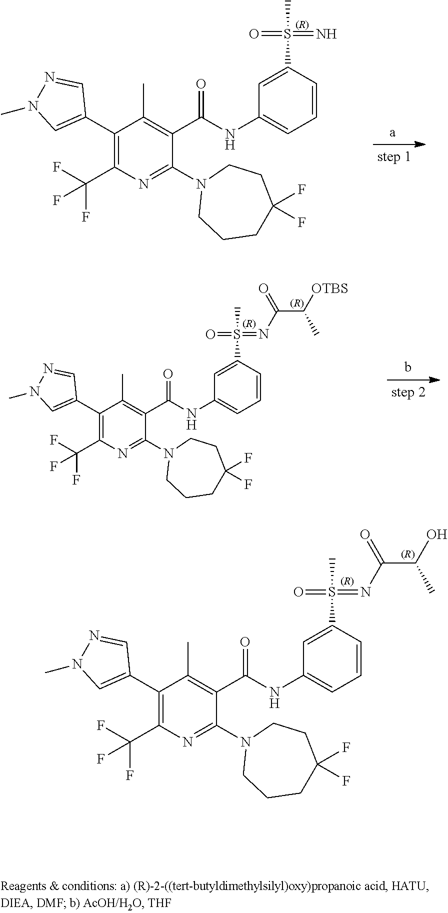

Step 1: N-(3-((R)-N-((R)-2-((tert-butyldimethylsilyl)oxy)propanoyl)-S-methylsulfonimidoyl)phenyl)-2-(4,4-difluoroazepan-1-yl)-4-methyl-5-(1-methyl-1H-pyrazol-4-yl)-6-(trifluoromethyl)nicotinamide: A mixture of (R)-2-(4,4-difluoroazepan-1-yl)-4-methyl-5-(1-methyl-1H-pyrazol-4-yl)-N-(3-(S-methylsulfonimidoyl)phenyl)-6-(trifluoromethyl)nicotinamide (100 mg, 0.18 mmol) and (R)-2-((tert-butyldimethylsilyl)oxy)propanoic acid (54 mg, 0.26 mmol) in DMF (5 mL) was added HATU (133 mg, 0.35 mmol) and DIEA (68 mg, 0.53 mmol). The mixture was stirred at room temperature for 16 hours. The reaction was monitored by LCMS. After the reaction was completed, the resulting solution was diluted with water (20 mL) and extracted with DCM (20 mL×3). The combined organic phases were washed with brine, dried over Na₂SO₄, concentrated under vacuum. The residue was purified by flash column chromatography on silica gel (PE/EtOAc=2/1 to 1/1) to give N-(3-((R)-N-((R)-2-((tert-butyldimethylsilyl)oxy)propanoyl)-S-methylsulfonimidoyl)phenyl)-2-(4,4-difluoroazepan-1-yl)-4-methyl-5-(1-methyl-1H-pyrazol-4-yl)-6-(trifluoromethyl)nicotinamide (66 mg, 49.7%) as a yellow solid. LCMS (ESI) calcd. for C₃₄H₄₆F₅N₆O₄SSi [M+H]⁺ m/z 757.30, found 757.10.

Step 2: 2-(4,4-difluoroazepan-1-yl)-N-(3-((R)-N-((R)-2-hydroxypropanoyl)-S-methylsulfonimidoyl)phenyl)-4-methyl-5-(1-methyl-1H-pyrazol-4-yl)-6-(trifluoromethyl)nicotinamide: A mixture of N-(3-((R)-N-((R)-2-((tert-butyldimethylsilyl)oxy)propanoyl)-S-methylsulfonimidoyl)phenyl)-2-(4,4-difluoroazepan-1-yl)-4-methyl-5-(1-methyl-1H-pyrazol-4-yl)-6-(trifluoromethyl)nicotinamide (66 mg, 0.09 mmol) in THF (3 mL) and H₂O (1 mL) was added AcOH (3 mL) at room temperature. The reaction mixture was stirred at room temperature for 2 hours. After the reaction was completed, the solution was diluted with water (30 mL) and extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 95% MeCN/H₂O containing 0.1% formic acid) to obtain 2-(4,4-difluoroazepan-1-yl)-N-(3-((R)-N-((R)-2-hydroxypropanoyl)-S-methylsulfonimidoyl)phenyl)-4-methyl-5-(1-methyl-1H-pyrazol-4-yl)-6-(trifluoromethyl)nicotinamide (13.2 mg, 23.5%) as a white solid. ¹H NMR (400 MHz, DMSO-d₆) δ 11.12 (s, 1H), 8.40 (s, 1H), 8.02-7.96 (m, 1H), 7.77-7.68 (m, 3H), 7.40 (s, 1H), 4.79 (d, J=5.3 Hz, 1H), 4.11-4.03 (m, 1H), 3.94 (s, 3H), 3.72 (s, 2H), 3.65 (t, J=5.9 Hz, 2H), 3.51 (s, 3H), 2.41-2.29 (m, 2H), 2.13-2.02 (m, 5H), 1.91-1.83 (m, 2H), 1.31 (d, J=6.8 Hz, 3H). LCMS (ESI) calcd. for C₂₈H₃₂F₅N₆O₄S [M+H]⁺ m/z 643.21, found 643.10.

### Example 237

### 4-methyl-N-(2-((R)—S-methylsulfonimidoyl)pyridin-4-yl)-5-(trifluoromethyl)-2-((S)-2-(trifluoromethyl)morpholino)nicotinamide

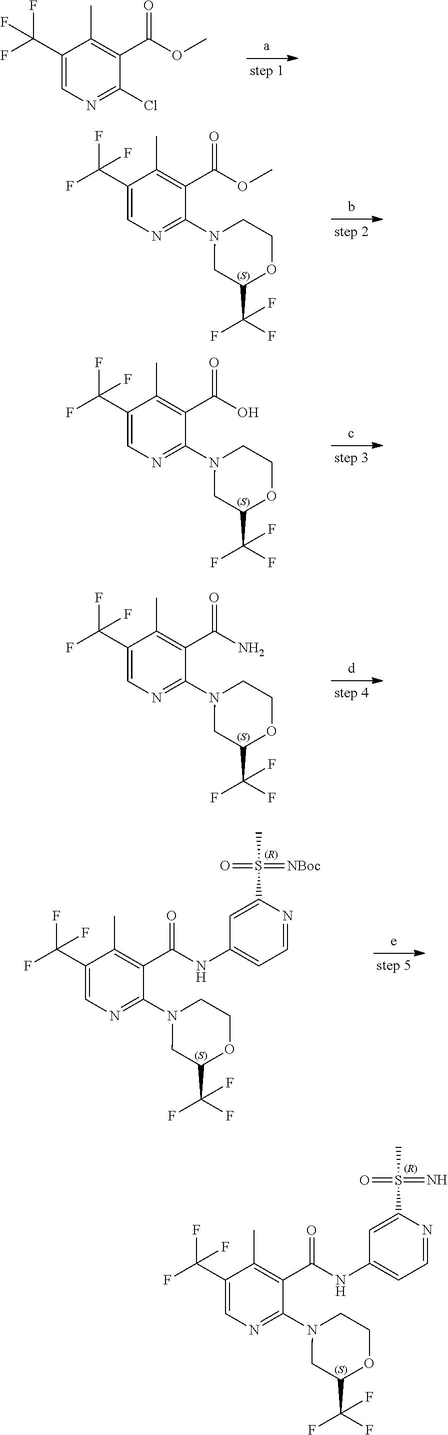

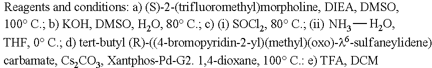

Step 1: methyl (S)-4-methyl-5-(trifluoromethyl)-2-(2-(trifluoromethyl)morpholino)nicotinate: A mixture of methyl 2-chloro-4-methyl-5-(trifluoromethyl)nicotinate (160 mg, 0.63 mmol), (S)-2-(trifluoromethyl)morpholine (196 mg, 1.26 mmol) and DIEA (815 mg, 6.31 mmol) in DMSO (3 mL) was heated to 100° C. and stirred for 5 hours. Then the mixture was quenched with water (10 mL) and extracted with DCM (10 mL×3). The combined organic layers were washed with brine, dried over sodium sulfate, concentrated under vacuum. The residue was purified by flash column chromatography on silica gel (PE/EtOAc=10/1) to afford methyl (S)-4-methyl-5-(trifluoromethyl)-2-(2-(trifluoromethyl)morpholino)nicotinate (110 mg, 46.90%) as a yellow solid. LCMS (ESI) calcd. for C₁₄H₁₈F₆N₂O₃ [M+H]⁺ m/z 373.10, found 372.9.

Step 2: (S)-4-methyl-5-(trifluoromethyl)-2-(2-(trifluoromethyl)morpholino)nicotinic acid: To a solution of methyl (S)-4-methyl-5-(trifluoromethyl)-2-(2-(trifluoromethyl)morpholino)nicotinate (110 mg, 0.30 mmol) in DMSO (2 mL) was added KOH (336 mg, 6.00 mmol) in H₂O (2 mL) at 25° C. The mixture was heated to 80° C. and stirred for 2 hours. After the reaction was completed, the aqueous phase was adjusted to pH=3-4 with 1N HCl, extracted with EtOAc (10 mL×3). The combined organic layers were washed with brine, dried with Na₂SO₄, and concentrated under vacuum to afford (S)-4-methyl-5-(trifluoromethyl)-2-(2-(trifluoromethyl)morpholino)nicotinic acid (100 mg, 93.1%) as yellow oil. LCMS (ESI) calcd. for C₁₃H₁₃F₆N₂O₃ [M+H]⁺ m/z 359.09, found 359.0.

Step 3: (S)-4-methyl-5-(trifluoromethyl)-2-(2-(trifluoromethyl)morpholino)nicotinamide: A solution of (S)-4-methyl-5-(trifluoromethyl)-2-(2-(trifluoromethyl)morpholino)nicotinic acid (100 mg, 0.28 mmol) in SOCl₂ (3 mL) was heated to 80° C. and stirred for 2 hours. The solution was concentrated under vacuum to provide the chloride intermediate. Then the chloride intermediate dissolved in THE (3 mL) was added to NH₃·H₂O (3 mL) at 0° C. The resulting mixture was stirred at 25° C. for 1 hour. Then the mixture was quenched with water (10 mL) and extracted with DCM (10 mL×3). The combined organic layers were washed with brine, dried over sodium sulfate, concentrated under vacuum. The residue was purified by flash column chromatography on silica gel (PE/EtOAc=3/2) to provide (S)-4-methyl-5-(trifluoromethyl)-2-(2-(trifluoromethyl)morpholino)nicotinamide (90 mg, 90.0%) as yellow oil. LCMS (ESI) calcd. for C₁₃H₁₄F₆N₃O₂ [M+H]⁺ m/z 358.10, found 357.9.

Step 4: tert-butyl ((R)-methyl(4-(4-methyl-5-(trifluoromethyl)-2-((S)-2-(trifluoromethyl)morpholino)nicotinamido)pyridin-2-yl)(oxo)-λ⁶-sulfaneylidene)carbamate: A solution of (S)-4-methyl-5-(trifluoromethyl)-2-(2-(trifluoromethyl)morpholino)nicotinamide (90 mg, 0.25 mmol), tert-butyl (R)-((4-bromopyridin-2-yl)(methyl)(oxo)-λ⁶-sulfaneylidene)carbamate (102 mg, 0.30 mmol), Cs₂CO₃ (246 mg, 0.76 mmol) and Xantphos-Pd-G2 (22 mg, 0.03 mmol) in 1,4-dioxane (5 mL) was heated to 100° C. and refluxed for 4 hours under nitrogen. After the reaction was completed, the mixture was concentrated. The residue was purified by flash column chromatography on silica gel (PE/EtOAc=1/1) to provide tert-butyl ((R)-methyl(4-(4-methyl-5-(trifluoromethyl)-2-((S)-2-(trifluoromethyl)morpholino)nicotinamido)pyridin-2-yl)(oxo)-λ⁶-sulfaneylidene)carbamate (90 mg, 58.9%) as a yellow solid. LCMS (ESI) calcd. for C₂₄H₂₈F₆N₅O₅S [M+H]⁺ m/z 612.17, found 612.0.

Step 5: 4-methyl-N-(2-((R)—S-methylsulfonimidoyl)pyridin-4-yl)-5-(trifluoromethyl)-2-((S)-2-(trifluoromethyl)morpholino)nicotinamide: A solution of tert-butyl ((R)-methyl(4-(4-methyl-5-(trifluoromethyl)-2-((S)-2-(trifluoromethyl)morpholino)nicotinamido)pyridin-2-yl)(oxo)-λ⁶-sulfaneylidene)carbamate (90 mg, 0.15 mmol) in DCM (5 mL) was added TFA (1 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (15 mL×3). The combined organic layers were washed with brine, dried over sodium sulfate, concentrated under vacuum. The residue was purified by prep-HPLC (Gemini 5 um C₁₈ column, 150*21.2 mm, eluting with 45% to 80% MeCN/H₂O containing 0.05% NH₃) to provide 4-methyl-N-(2-((R)—S-methylsulfonimidoyl)pyridin-4-yl)-5-(trifluoromethyl)-2-((S)-2-(trifluoromethyl)morpholino)nicotinamide (55.00 mg, 71.7%) as a white solid. ¹H NMR (400 MHz, DMSO-d₆) δ 11.40 (s, 1H), 8.68-8.57 (m, 2H), 8.39 (d, J=1.5 Hz, 1H), 7.83 (dd, J=5.4, 1.9 Hz, 1H), 4.39 (s, 1H), 4.24-4.12 (m, 2H), 4.00-3.92 (m, 1H), 3.83-3.74 (m, 1H), 3.60-3.50 (m, 1H), 3.25-3.11 (m, 5H), 2.36 (s, 3H). LCMS (ESI) calcd. for C₁₉H₂₀F₆N₅O₃S [M+H]⁺ m/z 512.12, found 512.1.

### Example 238

### 4-methyl-N-(3-((R)—S-methylsulfonimidoyl)phenyl)-5-(trifluoromethyl)-2-((R)-2-(trifluoromethyl)morpholino)nicotinamide

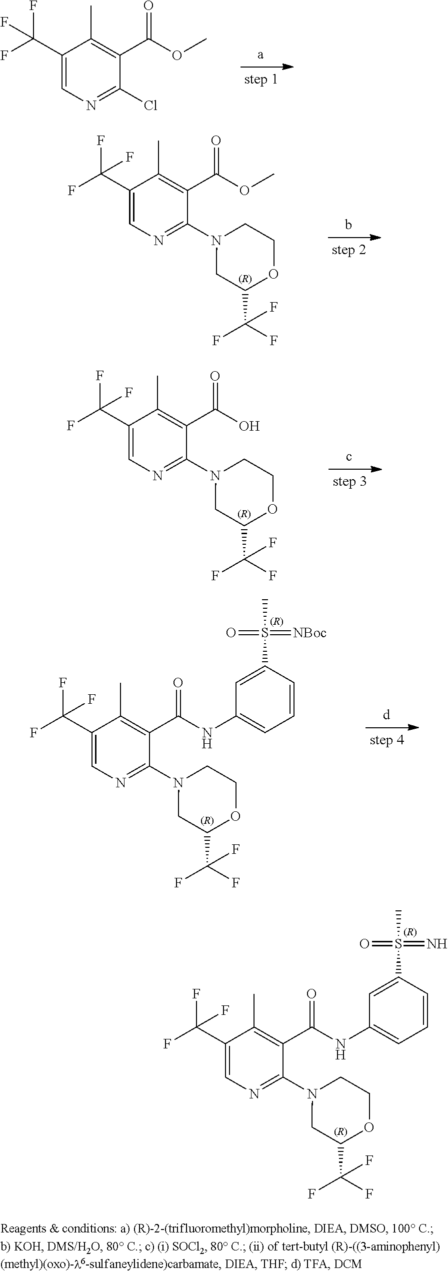

Step 1: methyl (R)-4-methyl-5-(trifluoromethyl)-2-(2-(trifluoromethyl)morpholino)nicotinate: A mixture of methyl 2-chloro-4-methyl-5-(trifluoromethyl)nicotinate (300 mg, 1.18 mmol) and (R)-2-(trifluoromethyl)morpholine (367 mg, 2.37 mmol) in DMSO (5 mL) was added DIEA (459 mg, 3.55 mmol) at room temperature. Then the reaction mixture was heated to 100° C. and stirred for 16 hours. After the reaction was completed, the resulting solution was diluted with water (15 mL) and extracted with EtOAc (15 mL×3). The combined organic layers were washed with brine, dried over Na₂SO₄, concentrated under vacuum. The residue was purified by flash column chromatography on silica gel (PE/EtAOc=1/1) to give methyl (R)-4-methyl-5-(trifluoromethyl)-2-(2-(trifluoromethyl)morpholino)nicotinate (300 mg, 68.1%) as a yellow solid. LCMS (ESI) calcd. for C₁₄H₁₅F₆N₂O₃ [M+H]⁺ m/z 373.10, found 373.1.

Step 2: (R)-4-methyl-5-(trifluoromethyl)-2-(2-(trifluoromethyl)morpholino)nicotinic acid: To a solution of methyl (R)-4-methyl-5-(trifluoromethyl)-2-(2-(trifluoromethyl)morpholino)nicotinate (300 mg, 0.81 mmol) in DMSO/H₂O (1/1, 10 mL) was added KOH (904 mg, 16.10 mmol). The reaction mixture was heated to 80° C. and stirred for 16 hours. After the reaction was completed, the mixture was concentrated. The residue was adjusted to pH=3-4 with aqueous HCl (1 M). Then the solution was extracted with EtOAc (20 mL×3). The combined organic layers were washed with brine, dried over sodium sulfate, concentrated under vacuum to provide (R)-4-methyl-5-(trifluoromethyl)-2-(2-(trifluoromethyl)morpholino)nicotinic acid (250 mg, 86.6%) as a yellow solid. LCMS (ESI) calcd. for C₁₃H₁₃F₆N₂O₃ [M+H]⁺ m/z 359.09, found 359.1.

Step 3: tert-butyl ((R)-methyl(3-(4-methyl-5-(trifluoromethyl)-2-((R)-2-(trifluoromethyl)morpholino)nicotinamido)phenyl)(oxo)-λ⁶-sulfaneylidene)carbamate: A solution of (R)-4-methyl-5-(trifluoromethyl)-2-(2-(trifluoromethyl)morpholino)nicotinic acid (120 mg, 0.34 mmol) in SOCl₂ (3 mL) was heated to 80° C. and stirred for 1 hour. The solution was concentrated under vacuum to provide the chloride intermediate. Then the chloride intermediate in THE (3 mL) was added to a stirred solution of tert-butyl (R)-((3-aminophenyl)(methyl)(oxo)-λ⁶-sulfaneylidene)carbamate (109 mg, 0.40 mmol) and DIEA (130 mg, 1.01 mmol) in THE (3 mL) at 25° C. The resulting mixture was heated to 50° C. and stirred for 16 hours. Then the mixture was quenched with water (20 mL) and extracted with EtOAc (20 mL×3). The combined organic layers were washed with brine, dried over sodium sulfate, concentrated under vacuum. The residue was purified by flash column chromatography on silica gel (PE/EtOAc=3/2) to provide tert-butyl ((R)-methyl(3-(4-methyl-5-(trifluoromethyl)-2-((R)-2-(trifluoromethyl)morpholino)nicotinamido)phenyl)(oxo)-λ⁶-sulfaneylidene)carbamate (90 mg, 44%) as a white solid. LCMS (ESI) calcd. for C₂₅H₂₉F₆N₄O₅S [M+H]⁺ m/z 611.18, found 611.1.

Step 4: 4-methyl-N-(3-((R)—S-methylsulfonimidoyl)phenyl)-5-(trifluoromethyl)-2-((R)-2-(trifluoromethyl)morpholino)nicotinamide: A solution of tert-butyl ((R)-methyl(3-(4-methyl-5-(trifluoromethyl)-2-((R)-2-(trifluoromethyl)morpholino)nicotinamido)phenyl)(oxo)-λ⁶-sulfaneylidene)carbamate (90 mg, 0.15 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (15 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 80% MeCN/H₂O containing 0.05% NH₃) to provide 4-methyl-N-(3-((R)—S-methylsulfonimidoyl)phenyl)-5-(trifluoromethyl)-2-((R)-2-(trifluoromethyl)morpholino)nicotinamide (24.00 mg, 31.8%) as a white solid. ¹H NMR (400 MHz, DMSO-d₆) δ 11.01 (s, 1H), 8.58 (s, 1H), 8.33 (s, 1H), 7.90 (d, J=8.1 Hz, 1H), 7.71 (d, J=7.9 Hz, 1H), 7.62 (t, J=7.9 Hz, 1H), 4.27-4.12 (m, 3H), 4.00-3.85 (m, 2H), 3.62-3.52 (m, 1H), 3.23-3.12 (m, 2H), 3.06 (s, 3H), 2.37 (s, 3H). LCMS (ESI) calcd. for C₂₀H₂₁F₆N₄O₃S [M+H]⁺ m/z 511.13, found 511.0.

### Example 239

### 4-methyl-N-(2-((R)—S-methylsulfonimidoyl)pyridin-4-yl)-5-(trifluoromethyl)-2-((R)-2-(trifluoromethyl)morpholino)nicotinamide

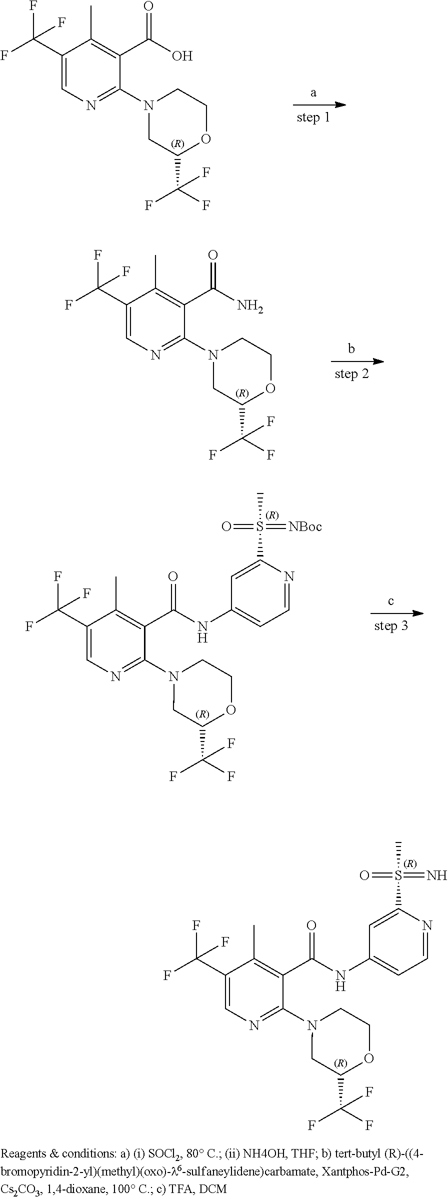

Step 1: (R)-4-methyl-5-(trifluoromethyl)-2-(2-(trifluoromethyl)morpholino)nicotinamide: A solution of (R)-4-methyl-5-(trifluoromethyl)-2-(2-(trifluoromethyl)morpholino)nicotinic acid (120 mg, 0.34 mmol) in SOCl₂ (3 mL) was heated to 80° C. and stirred for 1 hour. The solution was concentrated under vacuum to provide the chloride intermediate. Then the chloride intermediate in THF (3 mL) was added to a stirred solution of NH₄OH (3 mL) at 25° C. The resulting mixture was stirred at 25° C. for 2 hours. Then the mixture was quenched with water (20 mL) and extracted with EtOAc (20 mL×3). The combined organic layers were washed with brine, dried over sodium sulfate, concentrated under vacuum. The residue was purified by flash column chromatography on silica gel (PE/EtOAc=2/1) to provide (R)-4-methyl-5-(trifluoromethyl)-2-(2-(trifluoromethyl)morpholino)nicotinamide (100 mg, 83.5%) as a white solid. LCMS (ESI) calcd. for C₁₃H₁₄F₆N₃O₂ [M+H]⁺ m/z 358.10, found 357.9.

Step 2: tert-butyl ((R)-methyl(4-(4-methyl-5-(trifluoromethyl)-2-((R)-2-(trifluoromethyl)morpholino)nicotinamido)pyridin-2-yl)(oxo)-λ⁶-sulfaneylidene)carbamate: A solution of (R)-4-methyl-5-(trifluoromethyl)-2-(2-(trifluoromethyl)morpholino)nicotinamide (100 mg, 0.28 mmol) in 1,4-dioxane (5 mL) was added tert-butyl (R)-((4-bromopyridin-2-yl)(methyl)(oxo)-λ⁶-sulfaneylidene)carbamate (141 mg, 0.42 mmol), Cs₂CO₃ (274 mg, 0.84 mmol) and Xantphos-Pd-G2 (25 mg, 0.03 mmol) at room temperature under nitrogen. Then the reaction mixture was heated to 100° C. and stirred for 16 hours under nitrogen. The reaction was monitored by LCMS. After the reaction was completed, the filtrate was diluted with water (15 mL) and extracted with DCM (15 mL×3). The combine organic layers were washed with brine, dried over sodium sulfate, concentrated under vacuum. The residue was purified by flash column chromatography on silica gel (PE/EtOAc=1/1) to give tert-butyl ((R)-methyl(4-(4-methyl-5-(trifluoromethyl)-2-((R)-2-(trifluoromethyl)morpholino)nicotinamido)pyridin-2-yl)(oxo)-λ⁶-sulfaneylidene)carbamate (110 mg, 64.3%) as a yellow solid. LCMS (ESI) calcd. for C₂₄H₂₈F₆N₅O₅S [M+H]⁺ m/z 612.17, found 612.1.

Step 3: 4-methyl-N-(2-((R)—S-methylsulfonimidoyl)pyridin-4-yl)-5-(trifluoromethyl)-2-((R)-2-(trifluoromethyl)morpholino)nicotinamide: A solution of tert-butyl ((R)-methyl(4-(4-methyl-5-(trifluoromethyl)-2-((R)-2-(trifluoromethyl)morpholino)nicotinamido)pyridin-2-yl)(oxo)-λ⁶-sulfaneylidene)carbamate (110 mg, 0.18 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (15 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 80% MeCN/H₂O containing 0.05% NH₃) to provide 4-methyl-N-(2-((R)—S-methylsulfonimidoyl)pyridin-4-yl)-5-(trifluoromethyl)-2-((R)-2-(trifluoromethyl)morpholino)nicotinamide (37.00 mg, 40.2%) as a white solid. ¹H NMR (400 MHz, DMSO-d₆) δ 11.41 (s, 1H), 8.70-8.56 (m, 2H), 8.40 (d, J=1.7 Hz, 1H), 7.83 (dd, J=5.2, 1.9 Hz, 1H), 4.40 (s, 1H), 4.26-4.10 (m, 2H), 4.03-3.91 (m, 1H), 3.84-3.73 (m, 1H), 3.61-3.49 (m, 1H), 3.24-3.11 (m, 5H), 2.36 (s, 3H). LCMS (ESI) calcd. for C₁₉H₂₀F₆N₅O₃S [M+H]⁺ m/z 512.12, found 512.1.

### Example 240

### 4-methyl-N-(3-((R)—S-methylsulfonimidoyl)phenyl)-5-(trifluoromethyl)-2-((S)-2-(trifluoromethyl)morpholino)nicotinamide

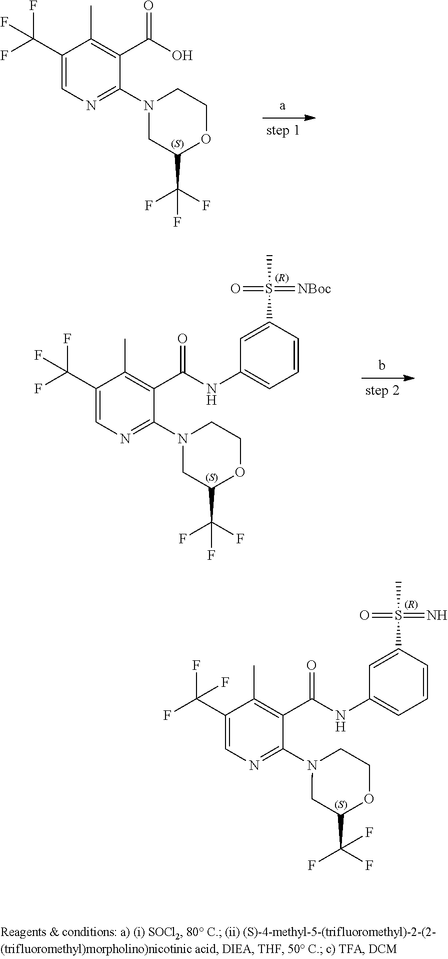

Step 1: tert-butyl ((R)-methyl(3-(4-methyl-5-(trifluoromethyl)-2-((S)-2-(trifluoromethyl)morpholino)nicotinamido)phenyl)(oxo)-λ⁶-sulfaneylidene)carbamate: A solution of (S)-4-methyl-5-(trifluoromethyl)-2-(2-(trifluoromethyl)morpholino)nicotinic acid (60 mg, 0.17 mmol) in SOCl₂ (1 mL) was heated at 80° C. for 2 hours. The solution was concentrated under vacuum to provide the chloride intermediate. Then the chloride intermediate dissolved in THF (1 mL) was added to a solution of tert-butyl (R)-((3-aminophenyl)(methyl)(oxo)-λ⁶-sulfaneylidene)carbamate (45.3 mg, 0.17 mmol) and DIEA (108.2 mg, 0.84 mmol) in THE (1 mL) at 0° C. The resulting mixture was stirred at 50° C. for 16 hours. Then the mixture was quenched with water (50 mL) and extracted with DCM (50 mL×3). The combined organic phases were washed with brine, dried over sodium sulfate, concentrated under vacuum. The residue was purified by flash column chromatography on silica gel (PE/EtOAc=1/1) to provide tert-butyl ((R)-methyl(3-(4-methyl-5-(trifluoromethyl)-2-((S)-2-(trifluoromethyl)morpholino)nicotinamido)phenyl)(oxo)-λ⁶-sulfaneylidene)carbamate (50 mg, 48.9%) as a white solid. LCMS (ESI) calcd. for C₂₅H₂₉F₆N₄O₅S [M+H]⁺ m/z 611.18, found 611.1.

Step 2: 4-methyl-N-(3-((R)—S-methylsulfonimidoyl)phenyl)-5-(trifluoromethyl)-2-((S)-2-(trifluoromethyl)morpholino)nicotinamide: A solution of tert-butyl ((R)-methyl(3-(4-methyl-5-(trifluoromethyl)-2-((S)-2-(trifluoromethyl)morpholino)nicotinamido)phenyl)(oxo)-λ⁶-sulfaneylidene)carbamate (50 mg, 0.08 mmol) in DCM (3 mL) was added TFA (0.5 mL) at room temperature. The reaction mixture was stirred at room temperature for 2 hours. After the reaction was completed, the solution was diluted with water (10 mL) and extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 45% to 80% MeCN/H₂O containing 0.05% NH₃) to obtain 4-methyl-N-(3-((R)—S-methylsulfonimidoyl)phenyl)-5-(trifluoromethyl)-2-((S)-2-(trifluoromethyl)morpholino)nicotinamide (6.00 mg, 14.7%) as a white solid. ¹H NMR (400 MHz, DMSO-d₆) δ 11.01 (s, 1H), 8.58 (s, 1H), 8.35-8.30 (m, 1H), 7.90 (d, J=8.0 Hz, 1H), 7.71 (d, J=7.9 Hz, 1H), 7.61 (t, J=7.9 Hz, 1H), 4.27-4.12 (m, 3H), 4.03-3.83 (m, 2H), 3.61-3.52 (m, 1H), 3.23-3.11 (m, 2H), 3.06 (s, 3H), 2.37 (s, 3H). LCMS (ESI) calcd. for C₂₀H₂₁F₆N₄O₃S [M+H]⁺ m/z 511.13, found 511.1.

### Example 241

### (R)-2-(4,4-difluoropiperidin-1-yl)-4-methyl-N-(3-(S-methylsulfonimidoyl)phenyl)-5-(trifluoromethyl)nicotinamide

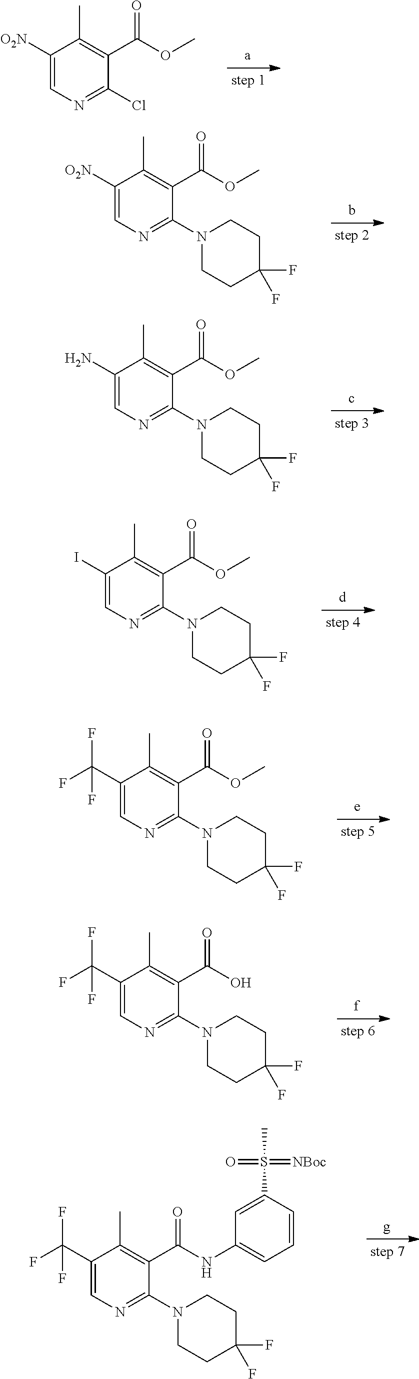

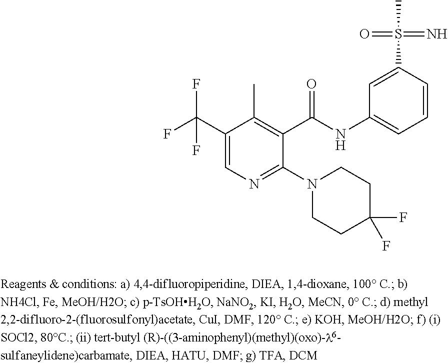 [SMILES: Cc1c(C(F)(F)F)cnc(N2CCC(F)(F)CC2)c1C(=O)Nc1cccc([S@](C)(=N)=O)c1]

Step 1: methyl 2-(4,4-difluoropiperidin-1-yl)-4-methyl-5-nitronicotinate: A solution of methyl 2-chloro-4-methyl-5-nitronicotinate (3 g, 13 mmol), 4,4-difluoropiperidine (3 g, 19.5 mmol) and DIEA (5 g, 39 mmol) in 1,4-dioxane (30 mL) was heated to 100° C. and stirred for 3 hours. LCMS showed the reaction was completed. The mixture was diluted with water and extracted with EtOAc (100 mL×3). The combined organic layers were washed with brine, dried over Na₂SO₄, and concentrated under vacuum. The residue was purified by flash column chromatography on silica gel (PE/EtOAc=10/1) to give methyl 2-(4,4-difluoropiperidin-1-yl)-4-methyl-5-nitronicotinate (3.1 g, 65.4%) as white solid. LCMS (ESI) calcd. for C₁₃H₁₆F₂N₃O₄ [M+H]⁺ m/z 316.11, found 315.9

Step 2: methyl 5-amino-2-(4,4-difluoropiperidin-1-yl)-4-methylnicotinate: A solution of methyl 2-(4,4-difluoropiperidin-1-yl)-4-methyl-5-nitronicotinate (3 g, 8.6 mmol) in MeOH (30 mL) and water (10 mL) was added NH₄Cl (2.3 g, 43 mmol), Fe (2.16 g, 38.7 mmol). The mixture was heated at 60° C. for 4 hours. After the reaction was completed. The mixture was filtered through celite. The filtrate was diluted with water (100 mL) and extracted with EtOAc (100 mL×3). The combined organic layers were washed with brine, dried over sodium sulfate, concentrated under vacuum to provide methyl 5-amino-2-(4,4-difluoropiperidin-1-yl)-4-methylnicotinate (1.5 g, 44.2%) as a yellow solid. LCMS (ESI) calcd. for C₁₃H₁₈F₂N₃O₂ [M+H]⁺ m/z 286.14, found 285.9.

Step 3: methyl 2-(4,4-difluoropiperidin-1-yl)-5-iodo-4-methylnicotinate: To a solution of NaNO₂ (0.87 g, 12.6 mmol) and KI (2.61 g, 15.7 mmol) in H₂O (4 mL) stirred at 0° C. was added a solution of p-TsOH·H₂O (3.6 g, 18.9 mmol) and methyl 5-amino-2-(4,4-difluoropiperidin-1-yl)-4-methylnicotinate (1.8 g, 6.3 mmol) in MeCN (4 mL) dropwise. The reaction mixture was stirred at 25° C. for 2 h. After the reaction was completed, the mixture was concentrated. The residue was adjusted to pH=8-9 with saturated aqueous NaHCO₃. Then the aqueous solution was extracted with EtOAc (30 mL×3). The combined organic layers were washed with brine, dried over sodium sulfate, concentrated under vacuum. The residue was purified by flash column chromatography on silica gel (PE/EtOAc=1/1) to give methyl 2-(4,4-difluoropiperidin-1-yl)-5-iodo-4-methylnicotinate (2 g, 79.3%) as yellow solid. LCMS (ESI) calcd. for C₁₃H₁₆F₂IN₂O₂[M+H]⁺ m/z 397.02, found 396.8.

Step 4: methyl 2-(4,4-difluoropiperidin-1-yl)-4-methyl-5-(trifluoromethyl)nicotinate: A solution of methyl 2-(4,4-difluoropiperidin-1-yl)-5-iodo-4-methylnicotinate (2 g, 5 mmol), methyl 2,2-difluoro-2-(fluorosulfonyl)acetate (4.80 mg, 25 mmol) and CuI (1.90 mg, 10 mmol) in DMF (50 mL) was heated to 120° C. and stirred for 5 hours under N₂. LCMS showed the reaction was completed. The mixture was diluted with water and extracted with EtOAc (50 mL×3). The combined organic layers were washed with brine, dried over Na₂SO₄, and concentrated under vacuum. The residue was purified by flash column chromatography on silica gel (PE/EtOAc=1/1) to give methyl 2-(4,4-difluoropiperidin-1-yl)-4-methyl-5-(trifluoromethyl)nicotinate (1.3 g, 76%) as yellow oil. LCMS (ESI) calcd. for C₁₄H₁₆F₅N₂O₂[M+H]⁺ m/z 339.12, found 396.8

Step 5: 2-(4,4-difluoropiperidin-1-yl)-4-methyl-5-(trifluoromethyl)nicotinic acid: To a solution of methyl 2-(4,4-difluoropiperidin-1-yl)-4-methyl-5-(trifluoromethyl)nicotinate (1.3 g, 3.8 mmol) and KOH (2.13 g, 38 mmol) in MeOH/H₂O (1/1, 20 mL) was heated to 80° C. and stirred for 16 hours. After the reaction was completed, the mixture was cooled and adjusted to pH=3-4 with 1N HCl and then extracted with EtOAc (20 mL×3). The combined organic layers were washed with brine, dried with Na₂SO₄, concentrated under reduced pressure to give 2-(4,4-difluoropiperidin-1-yl)-4-methyl-5-(trifluoromethyl)nicotinic acid (1 g, 81.5%) as yellow oil which was used without further purification. LCMS (ESI) calcd. for C₁₃H₁₄F₅N₂O₂ [M+H]⁺ m/z 325.10, found 325.1.

Step 6: tert-butyl (R)-((3-(2-(4,4-difluoropiperidin-1-yl)-4-methyl-5-(trifluoromethyl)nicotinamido)phenyl)(methyl)(oxo)-λ⁶-sulfaneylidene)carbamate: A solution of 2-(4,4-difluoropiperidin-1-yl)-4-methyl-5-(trifluoromethyl)nicotinic acid (200 mg, 0.61 mmol) in SOCl₂ (5 mL) was heated to 80° C. and stirred for 2 hours. The solution was concentrated under vacuum to provide the chloride intermediate. Then the chloride intermediate dissolved in THF (5 mL) was added to a solution of tert-butyl (R)-((3-aminophenyl)(methyl)(oxo)-λ⁶-sulfaneylidene)carbamate (249 mg, 0.92 mmol) and DIEA (238 mg, 1.84 mmol) in THE (5 mL). The resulting mixture was stirred at 50° C. for 16 hours. Then the mixture was quenched with water (20 mL) and then extracted with DCM (20 mL×3). The combined organic layers were washed with brine, dried over sodium sulfate, concentrated under vacuum. The residue was purified by flash column chromatography on silica gel (PE/EtOAc=1/1 to 1/2) to provide tert-butyl (R)-((3-(2-(4,4-difluoropiperidin-1-yl)-4-methyl-5-(trifluoromethyl)nicotinamido)phenyl)(methyl)(oxo)-λ⁶-sulfaneylidene)carbamate (200 mg, 56.32%) as a yellow solid. LCMS (ESI) calcd. for C₂₅H₃₀F₅N₄O₄S [M+H]⁺ m/z 577.19, found 0.577.1

Step 7: (R)-2-(4,4-difluoropiperidin-1-yl)-4-methyl-N-(3-(S-methylsulfonimidoyl)phenyl)-5-(trifluoromethyl)nicotinamide: A solution of tert-butyl (R)-((3-(2-(4,4-difluoropiperidin-1-yl)-4-methyl-5-(trifluoromethyl)nicotinamido)phenyl)(methyl)(oxo)-λ⁶-sulfaneylidene)carbamate (200 mg, 0.35 mmol) in DCM (10 mL) was added TFA (1 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (15 mL×3). The combined organic layers were washed with brine, dried over sodium sulfate, concentrated under vacuum. The residue was purified by prep-HPLC (Gemini 5 um C₁₈ column, 150*21.2 mm, eluting with 55% to 80% MeCN/H₂O containing 0.05% NH₃) to provide (R)-2-(4,4-difluoropiperidin-1-yl)-4-methyl-N-(3-(S-methylsulfonimidoyl)phenyl)-5-(trifluoromethyl)nicotinamide (82.8 mg, 49.6%) as a white solid. ¹H NMR (400 MHz, DMSO-d₆) δ 10.98 (s, 1H), 8.53 (s, 1H), 8.35 (s, 1H), 7.89 (d, J=8.1 Hz, 1H), 7.70 (d, J=7.8 Hz, 1H), 7.61 (t, J=7.9 Hz, 1H), 4.24 (s, 1H), 3.73-3.52 (m, 4H), 3.07 (s, 3H), 2.36 (s, 3H), 2.05-1.90 (m, 4H). LCMS (ESI) calcd. for C₂₀H₂₂F₅N₄O₂S [M+H]⁺ m/z 477.14, found 477.0.

### Example 242

### (R)-4-methyl-N-(3-(S-methylsulfonimidoyl)phenyl)-5-(trifluoromethyl)-2-(4-(trifluoromethyl)piperidin-1-yl)nicotinamide

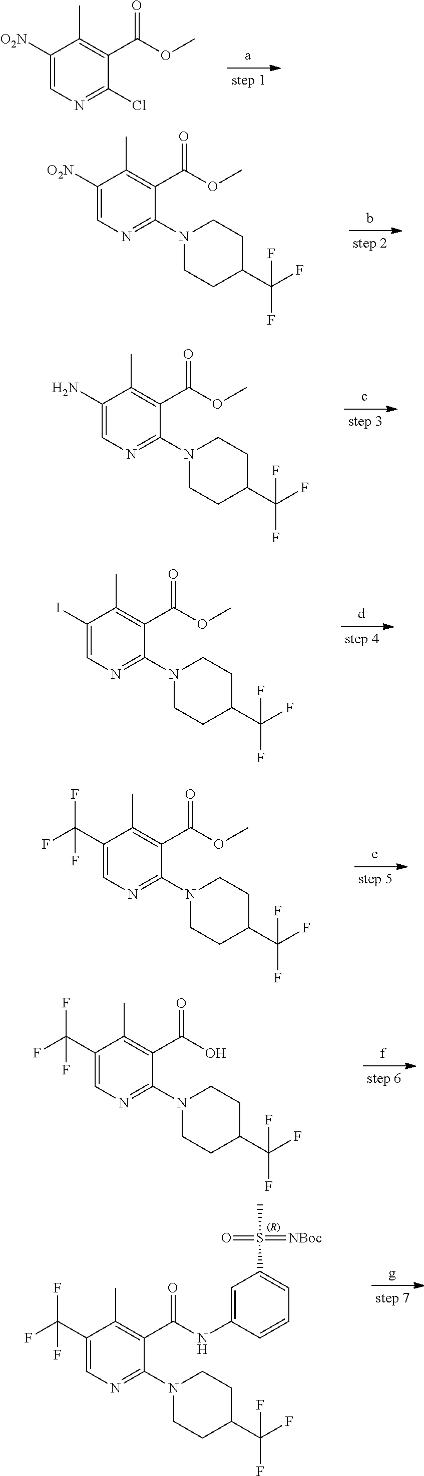

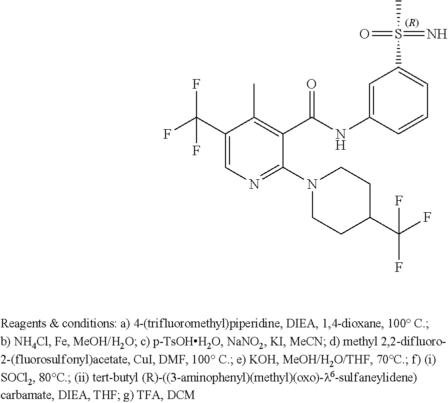

Step 1: methyl 4-methyl-5-nitro-2-(4-(trifluoromethyl)piperidin-1-yl)nicotinate: A solution of methyl 2-chloro-4-methyl-5-nitronicotinate (3 g, 13 mmol), 4-(trifluoromethyl)piperidine (3 g, 19.5 mmol) and DIEA (5 g, 39 mmol) in 1,4-dioxane (30 mL) was heated to 100° C. and stirred for 3 hours. LCMS showed the reaction was completed. The mixture was diluted with water and extracted with EtOAc (100 mL×3). The combined organic layers were washed with brine, dried over Na₂SO₄, and concentrated under vacuum. The residue was purified by flash column chromatography on silica gel (PE/EtOAc=10/1) to give methyl 4-methyl-5-nitro-2-(4-(trifluoromethyl)piperidin-1-yl)nicotinate (3.1 g, 65.4%) as white solid. LCMS (ESI) calcd. for C₁₄H₁₇F₃N₃O₄ [M+H]⁺ m/z 348.12, found 348.1.

Step 2: methyl 5-amino-4-methyl-2-(4-(trifluoromethyl)piperidin-1-yl)nicotinate: A solution of methyl 4-methyl-5-nitro-2-(4-(trifluoromethyl)piperidin-1-yl)nicotinate (3 g, 8.6 mmol) in MeOH (30 mL) and water (10 mL) was added NH₄Cl (2.3 g, 43 mmol), Fe (2.16 g, 38.7 mmol). The mixture was heated at 60° C. for 4 hours. After the reaction was completed. The mixture was filtered through celite. The filtrate was diluted with water (100 mL) and extracted with EtOAc (100 mL×3). The combined organic layers were washed with brine, dried over sodium sulfate, concentrated under vacuum to provide methyl 5-amino-4-methyl-2-(4-(trifluoromethyl)piperidin-1-yl)nicotinate (1.5 g, 44.2%) as a yellow solid. LCMS (ESI) calcd. for C₁₄H₁₉F₃N₃O₂ [M+H]⁺ m/z 318.15, found 318.1.

Step 3: methyl 5-iodo-4-methyl-2-(4-(trifluoromethyl)piperidin-1-yl)nicotinate: To a solution of methyl 5-amino-4-methyl-2-(4-(trifluoromethyl)piperidin-1-yl)nicotinate (1.5 g, 4.7 mmol) and p-TsOH·H₂O (2.68 g, 14.1 mmol) in MeCN (18 mL) stirred at 0° C. was added a solution of NaNO₂ (0.65 g, 9.4 mmol) and KI (1.95 g, 11.8 mmol) in H₂O (4 mL) dropwise. The reaction mixture was stirred at 25° C. for 2 h. After the reaction was completed, the mixture was concentrated. The residue was adjusted to pH=8-9 with saturated aqueous NaHCO₃. Then the aqueous solution was extracted with EtOAc (30 mL×3). The combined organic layers were washed with brine, dried over sodium sulfate, concentrated under vacuum. The residue was purified by flash column chromatography on silica gel (PE/EtOAc=10/1) to give methyl 5-iodo-4-methyl-2-(4-(trifluoromethyl)piperidin-1-yl)nicotinate (1.2 g, 57.5%) as yellow solid. LCMS (ESI) calcd. for C₁₄H₁₇F₃IN₂O₂ [M+H]⁺ m/z 429.03, found 429.0.

Step 4: methyl 4-methyl-5-(trifluoromethyl)-2-(4-(trifluoromethyl)piperidin-1-yl)nicotinate: A solution of methyl 5-iodo-4-methyl-2-(4-(trifluoromethyl)piperidin-1-yl)nicotinate (1.2 g, 2.8 mmol), methyl 2,2-difluoro-2-(fluorosulfonyl)acetate (2.69 g, 14 mmol) and CuI (1.07 mg, 5.6 mmol) in DMF (15 mL) was heated to 100° C. and stirred for 5 hours under N₂. LCMS showed the reaction was completed. The mixture was diluted with water and extracted with EtOAc (50 mL×3). The combined organic layers were washed with brine, dried over Na₂SO₄, and concentrated under vacuum. The residue was purified by flash column chromatography on silica gel (PE/EtOAc=10/1) to give methyl 4-methyl-5-(trifluoromethyl)-2-(4-(trifluoromethyl)piperidin-1-yl)nicotinate (0.8 g, 71.4%) as yellow oil. LCMS (ESI) calcd. for C₁₅H₁₇F₆N₂O₂ [M+H]⁺ m/z 371.12, found 371.1.

Step 5: 4-methyl-5-(trifluoromethyl)-2-(4-(trifluoromethyl)piperidin-1-yl)nicotinic acid: To a solution of methyl 4-methyl-5-(trifluoromethyl)-2-(4-(trifluoromethyl)piperidin-1-yl)nicotinate (800 mg, 2.16 mmol) and KOH (1.2 g, 21.6 mmol) in MeOH/H₂O/THF (1/1/1, 25 mL) was heated to 70° C. and stirred for 16 hours. After the reaction was completed, the mixture was cooled and adjusted to pH=3-4 with 1N HCl and then extracted with EtOAc (20 mL×3). The combined organic layers were washed with brine, dried with Na₂SO₄, concentrated under reduced pressure to give 4-methyl-5-(trifluoromethyl)-2-(4-(trifluoromethyl)piperidin-1-yl)nicotinic acid (750 mg, 97.3% yield) as yellow oil which was used without further purification. LCMS (ESI) calcd. for C₁₄H₁₅F₆N₂O₂ [M+H]⁺ m/z 357.11, found 356.9.

Step 6: tert-butyl (R)-(methyl(3-(4-methyl-5-(trifluoromethyl)-2-(4-(trifluoromethyl)piperidin-1-yl)nicotinamido)phenyl)(oxo)-λ⁶-sulfaneylidene)carbamate: A solution of 4-methyl-5-(trifluoromethyl)-2-(4-(trifluoromethyl)piperidin-1-yl)nicotinic acid (200 mg, 0.56 mmol) in SOCl₂ (5 mL) was heated to 80° C. and stirred for 2 hours. The solution was concentrated under vacuum to provide the chloride intermediate. Then the chloride intermediate dissolved in THE (5 mL) was added to a solution of tert-butyl (R)-((3-aminophenyl)(methyl)(oxo)-λ⁶-sulfaneylidene)carbamate (104.8 mg, 0.62 mmol) and DIEA (361.8 mg, 2.80 mmol) in THE (5 mL). The resulting mixture was heated at 50° C. for 16 hours. Then the mixture was quenched with water (20 mL) and then extracted with DCM (20 mL×3). The combined organic layers were washed with brine, dried over sodium sulfate, concentrated under vacuum. The residue was purified by flash column chromatography on silica gel (PE/EtOAc=1/1) to provide tert-butyl (R)-(methyl(3-(4-methyl-5-(trifluoromethyl)-2-(4-(trifluoromethyl)piperidin-1-yl)nicotinamido)phenyl)(oxo)-λ⁶-sulfaneylidene)carbamate (200 mg, 58.7%) as a yellow solid. LCMS (ESI) calcd. for C₂₆H₃₁F₆N₄O₄S [M+H]⁺ m/z 609.20, found 609.0

Step 7: (R)-4-methyl-N-(3-(S-methylsulfonimidoyl)phenyl)-5-(trifluoromethyl)-2-(4-(trifluoromethyl)piperidin-1-yl)nicotinamide: A solution of tert-butyl (R)-(methyl(3-(4-methyl-5-(trifluoromethyl)-2-(4-(trifluoromethyl)piperidin-1-yl)nicotinamido)phenyl)(oxo)-λ⁶-sulfaneylidene)carbamate (200 mg, 0.33 mmol) in DCM (10 mL) was added TFA (1 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (15 mL×3). The combined organic layers were washed with brine, dried over sodium sulfate, concentrated under vacuum. The residue was purified by prep-HPLC (Gemini 5 um C₁₈ column, 150*21.2 mm, eluting with 40% to 80% MeCN/H₂O containing 0.05% NH₃) to provide (R)-4-methyl-N-(3-(S-methylsulfonimidoyl)phenyl)-5-(trifluoromethyl)-2-(4-(trifluoromethyl)piperidin-1-yl)nicotinamide(75.2 mg, 45.0%) as a white solid. ¹H NMR (400 MHz, DMSO-d₆) δ 10.93 (s, 1H), 8.50 (s, 1H), 8.35 (s, 1H), 7.89 (d, J=8.1 Hz, 1H), 7.69 (d, J=7.9 Hz, 1H), 7.60 (t, J=7.9 Hz, 1H), 4.25-4.13 (m, 3H), 3.05 (s, 3H), 3.03-2.93 (m, 2H), 2.63-2.52 (m, 1H), 2.35 (s, 3H), 1.85-1.74 (m, 2H), 1.48-1.32 (m, 2H). LCMS (ESI) calcd. for C₂₁H₂₃F₆N₄O₂S [M+H]⁺ m/z 509.15, found 509.1.

### Example 243

### (R)-5-chloro-N-(2-(S-methylsulfonimidoyl)pyridin-4-yl)-4-(trifluoromethyl)-2-(4-(trifluoromethyl)piperidin-1-yl)benzamide

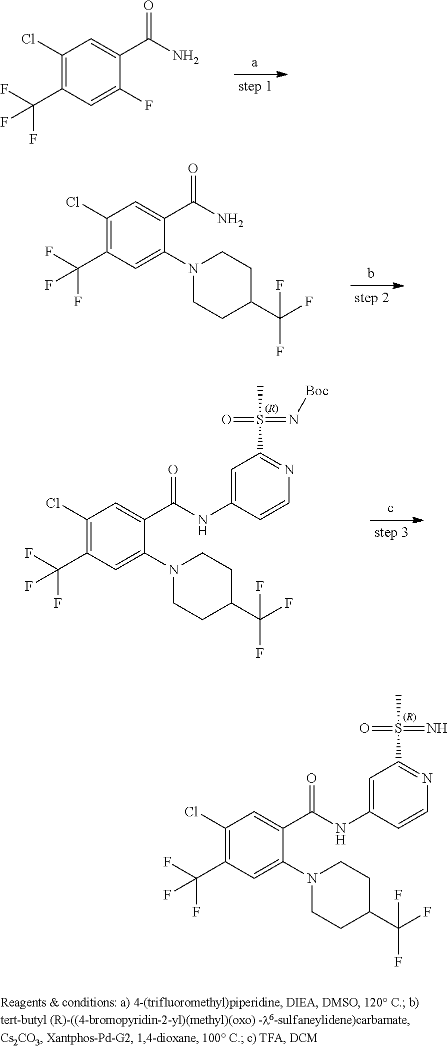

Step 1: 5-chloro-4-(trifluoromethyl)-2-(4-(trifluoromethyl)piperidin-1-yl)benzamide: A solution of 5-chloro-2-fluoro-4-(trifluoromethyl)benzamide (3 g, 12.4 mmol), 4-(trifluoromethyl)piperidine (1.99 g, 13.0 mmol), and DIEA (4.01 g, 31.0 mmol) in DMSO (50 mL) was heated to 120° C. and stirred for 16 hours. After the reaction was completed, the mixture was cooled to room temperature. The resulting solution was diluted with water (100 mL) and extracted with EtOAc (100 mL×3). The combined organic layers were washed with brine, dried over sodium sulfate, concentrated under vacuum. The residue was purified by flash column chromatography on silica gel (PE/EtOAc=1/1) to provide 5-chloro-4-(trifluoromethyl)-2-(4-(trifluoromethyl)piperidin-1-yl)benzamide (3.0 g, 64.52%) as yellow solid. LCMS (ESI) calcd. for C₁₄H₁₄ClF₆N₂O [M+H]⁺ m/z 375.07, found 375.0.

Step 2: tert-butyl (R)-((4-(5-chloro-4-(trifluoromethyl)-2-(4-(trifluoromethyl)piperidin-1-yl)benzamido)pyridin-2-yl)(methyl)(oxo)-λ⁶-sulfaneylidene)carbamate: A mixture of 5-chloro-4-(trifluoromethyl)-2-(4-(trifluoromethyl)piperidin-1-yl)benzamide (200 mg, 0.53 mmol), tert-butyl (R)-((4-bromopyridin-2-yl)(methyl)(oxo)-λ⁶-sulfaneylidene)carbamate (187 mg, 0.56 mmol), Cs₂CO₃ (452 mg, 1.39 mmol) and Xantphos-Pd-G2 (95 mg, 0.11 mmol) in 1,4-dioxane (5 mL) was stirred at 100° C. for 2 hours under nitrogen. After the reaction was completed, the mixture was filtered through celite. The filtrate was concentrated and residue was purified by flash column chromatography on silica gel (PE/EtOAc=1/1) to provide tert-butyl (R)-((4-(5-chloro-4-(trifluoromethyl)-2-(4-(trifluoromethyl)piperidin-1-yl)benzamido)pyridin-2-yl)(methyl)(oxo)-λ⁶-sulfaneylidene)carbamate (200 mg, 59.68% yield) as a yellow solid. LCMS (ESI) calcd. for C₂₅H₂₈ClF₆N₄O₄S [M+H]⁺ m/z 629.14, found 629.1.

Step 3: (R)-5-chloro-N-(2-(S-methylsulfonimidoyl)pyridin-4-yl)-4-(trifluoromethyl)-2-(4-(trifluoromethyl)piperidin-1-yl)benzamide: A solution of tert-butyl (R)-((4-(5-chloro-4-(trifluoromethyl)-2-(4-(trifluoromethyl)piperidin-1-yl)benzamido)pyridin-2-yl)(methyl)(oxo)-λ⁶-sulfaneylidene)carbamate (200 mg, 0.32 mmol) in DCM (5 mL) was added TFA (0.5 mL) at room temperature. The reaction mixture was stirred at room temperature for 1 hour. After the reaction was completed, the mixture was concentrated. The residue was adjusted to pH=8-9 with saturated aqueous NaHCO₃. Then the aqueous solution was extracted with DCM (15 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.05% NH₃) to provide (R)-5-chloro-N-(2-(S-methylsulfonimidoyl)pyridin-4-yl)-4-(trifluoromethyl)-2-(4-(trifluoromethyl)piperidin-1-yl)benzamide (58.40 mg, 34.78%) as a white solid. ¹H NMR (400 MHz, DMSO-d₆) δ 11.24 (s, 1H), 8.64 (d, J=5.4 Hz, 1H), 8.50 (s, 1H), 7.84 (s, 1H), 7.78 (dd, J=5.4, 1.7 Hz, 1H), 7.49 (s, 1H), 4.36 (s, 1H), 3.36 (d, J=11.8 Hz, 2H), 3.14 (s, 3H), 2.90 (t, J=12.0 Hz, 2H), 2.47-2.35 (m, 1H), 1.84 (d, J=11.9 Hz, 2H), 1.56-1.38 (m, 2H). LCMS (ESI) calcd. for C₂₀H₂₀ClF₆N₄O₂S [M+H]⁺ m/z 529.09, found 529.1.

### Example 244

### (S)-5-chloro-N-(2-(S-methylsulfonimidoyl)pyridin-4-yl)-4-(trifluoromethyl)-2-(4-(trifluoromethyl)piperidin-1-yl)benzamide

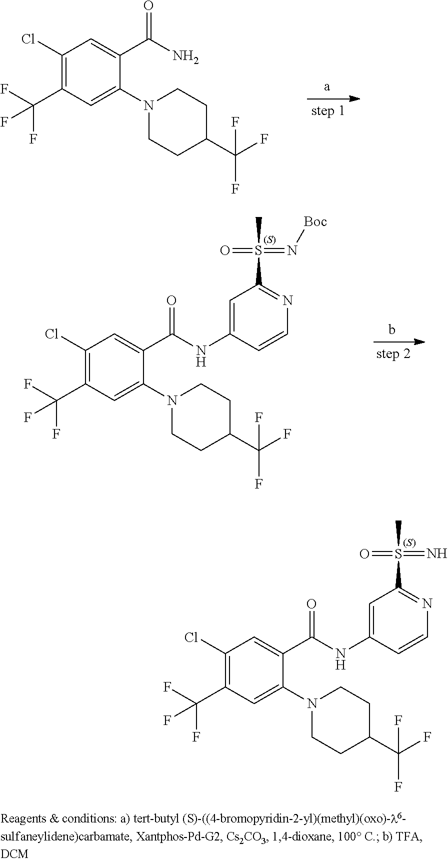

Step 1: tert-butyl (S)-((4-(5-chloro-4-(trifluoromethyl)-2-(4-(trifluoromethyl)piperidin-1-yl)benzamido)pyridin-2-yl)(methyl)(oxo)-λ⁶-sulfaneylidene)carbamate: A mixture of 5-chloro-4-(trifluoromethyl)-2-(4-(trifluoromethyl)piperidin-1-yl)benzamide (200 mg, 0.53 mmol), tert-butyl (S)-((4-bromopyridin-2-yl)(methyl)(oxo)-λ⁶-sulfaneylidene)carbamate (187 mg, 0.56 mmol), Cs₂CO₃ (452 mg, 1.39 mmol) and Xantphos-Pd-G2 (95 mg, 0.11 mmol) in 1,4-dioxane (5 mL) was heated to 100° C. and refluxed for 2 hours under nitrogen. After the reaction was completed, the mixture was filtered through celite. The filtrate was concentrated and residue was purified by flash column chromatography on silica gel (PE/EtOAc=1/1) to provide tert-butyl (S)-((4-(5-chloro-4-(trifluoromethyl)-2-(4-(trifluoromethyl)piperidin-1-yl)benzamido)pyridin-2-yl)(methyl)(oxo)-16-sulfaneylidene)carbamate (200 mg, 59.68%) as a yellow solid. LCMS (ESI) calcd. for C₂₅H₂₈ClF₆N₄O₄S [M+H]⁺ m/z 629.14, found 629.1.

Step 2: (S)-5-chloro-N-(2-(S-methylsulfonimidoyl)pyridin-4-yl)-4-(trifluoromethyl)-2-(4-(trifluoromethyl)piperidin-1-yl)benzamide: A solution of tert-butyl (S)-((4-(5-chloro-4-(trifluoromethyl)-2-(4-(trifluoromethyl)piperidin-1-yl)benzamido)pyridin-2-yl)(methyl)(oxo)-λ⁶-sulfaneylidene)carbamate (200 mg, 0.32 mmol) in DCM (5 mL) was added TFA (0.5 mL) at room temperature. The reaction mixture was stirred at room temperature for 1 hour. After the reaction was completed, the mixture was concentrated. The residue was adjusted to pH=8-9 with saturated aqueous NaHCO₃. Then the aqueous solution was extracted with DCM (15 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.05% NH₃) to provide (S)-5-chloro-N-(2-(S-methylsulfonimidoyl)pyridin-4-yl)-4-(trifluoromethyl)-2-(4-(trifluoromethyl)piperidin-1-yl)benzamide (84.30 mg, 50.18%) as a white solid. ¹H NMR (400 MHz, DMSO-d₆) δ 11.24 (s, 1H), 8.64 (d, J=5.4 Hz, 1H), 8.51 (s, 1H), 7.84 (s, 1H), 7.78 (dd, J=5.3, 1.6 Hz, 1H), 7.49 (s, 1H), 4.36 (s, 1H), 3.36 (d, J=11.8 Hz, 2H), 3.14 (s, 3H), 2.90 (t, J=12.0 Hz, 2H), 2.46-2.38 (m, 1H), 1.85 (d, J=11.9 Hz, 2H), 1.57-1.36 (m, 2H). LCMS (ESI) calcd. for C₂₀H₂₀ClF₆N₄O₂S [M+H]⁺ m/z 529.09, found 529.1.

### Example 245

### (R)-5-chloro-N-(4-fluoro-3-(S-methylsulfonimidoyl)phenyl)-4-(trifluoromethyl)-2-(4-(trifluoromethyl)piperidin-1-yl)benzamide

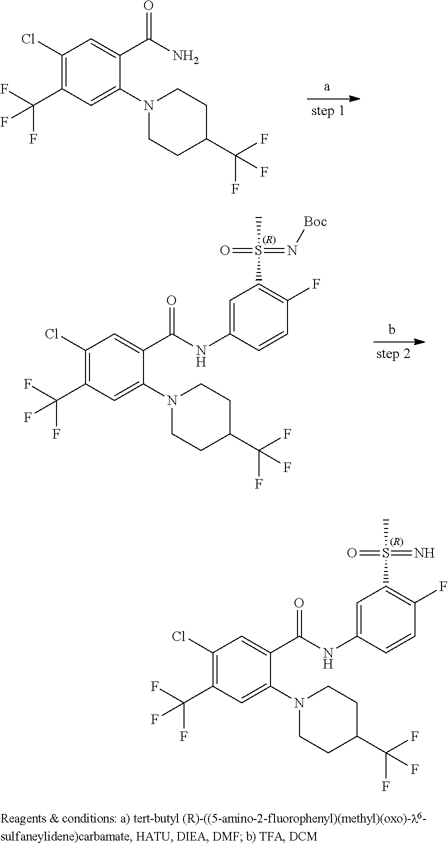

Step 1: tert-butyl (R)-((5-(5-chloro-4-(trifluoromethyl)-2-(4-(trifluoromethyl)piperidin-1-yl)benzamido)-2-fluorophenyl)(methyl)(oxo)-λ⁶-sulfaneylidene)carbamate: A mixture of 5-chloro-4-(trifluoromethyl)-2-(4-(trifluoromethyl)piperidin-1-yl)benzoic acid (150 mg, 0.40 mmol), tert-butyl (R)-((5-amino-2-fluorophenyl)(methyl)(oxo)-λ⁶-sulfaneylidene)carbamate (138 mg, 0.48 mmol), HATU (273 mg, 0.72 mmol) and DIEA (155 mg, 1.20 mmol) in DMF (5 mL) was stirred at room temperature for 16 hours. After the reaction was completed, the resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtOAc=2/1 to 1/1) to give tert-butyl (R)-((5-(5-chloro-4-(trifluoromethyl)-2-(4-(trifluoromethyl)piperidin-1-yl)benzamido)-2-fluorophenyl)(methyl)(oxo)-λ⁶-sulfaneylidene)carbamate (200 mg, 69.8%) as a yellow solid. LCMS (ESI) calcd. for C₂₆H₂₈ClF₇N₃O₄S [M+H]⁺ m/z 646.14, found 646.1.

Step 2: (R)-5-chloro-N-(4-fluoro-3-(S-methylsulfonimidoyl)phenyl)-4-(trifluoromethyl)-2-(4-(trifluoromethyl)piperidin-1-yl)benzamide: A solution of tert-butyl (R)-((5-(5-chloro-4-(trifluoromethyl)-2-(4-(trifluoromethyl)piperidin-1-yl)benzamido)-2-fluorophenyl)(methyl)(oxo)-λ⁶-sulfaneylidene)carbamate (200 mg, 0.37 mmol) in DCM (5 mL) was added TFA (0.5 mL) at room temperature. The reaction mixture was stirred at room temperature for 3 hours. After the reaction was completed, the mixture was concentrated. The residue was adjusted to pH=8-9 with saturated aqueous NaHCO₃. Then the aqueous solution was extracted with DCM (15 mL×3). The combined organic layers were washed with brine, dried over sodium sulfate, concentrated under vacuum. The residue was purified by prep-IPLC (Gemini 5 um C₁₈ column, 150*21.2 mm, eluting with 50% to 95% MeCN/H₂O containing 0.05% NH₃) to provide (R)-5-chloro-N-(4-fluoro-3-(S-methylsulfonimidoyl)phenyl)-4-(trifluoromethyl)-2-(4-(trifluoromethyl)piperidin-1-yl)benzamide (60 mg, 29.7%) as a white solid. ¹H NMR (400 MHz, DMSO-d₆, ppm) δ 10.88 (s, 1H), 8.38 (dd, J=6.5, 2.6 Hz, 1H), 7.96-7.84 (m, 1H), 7.80 (s, 1H), 7.52-7.43 (m, 2H), 4.67 (s, 1H), 3.40-3.86 (m, 2H), 3.19 (s, 3H), 2.91-2.86 (m, 2H), 2.52-2.47 (m, 1H), 1.87-1.83 (m, 2H), 1.57-1.39 (m, 2H). LCMS (ESI) calcd. for C₂₁H₂₀ClF₇N₃O₂S [M+H]⁺ m/z 546.08, found 546.1.

### Example 246

### (S)-5-chloro-N-(4-fluoro-3-(S-methylsulfonimidoyl)phenyl)-4-(trifluoromethyl)-2-(4-(trifluoromethyl)piperidin-1-yl)benzamide

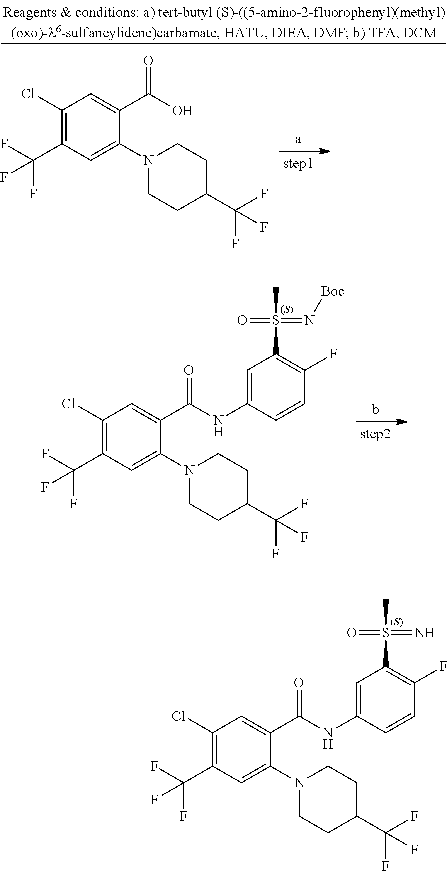

Step 1: tert-butyl (S)-((5-(5-chloro-4-(trifluoromethyl)-2-(4-(trifluoromethyl)piperidin-1-yl)benzamido)-2-fluorophenyl)(methyl)(oxo)-λ⁶-sulfaneylidene)carbamate: A mixture of 5-chloro-4-(trifluoromethyl)-2-(4-(trifluoromethyl)piperidin-1-yl)benzoic acid (150 mg, 0.40 mmol), tert-butyl (S)-((5-amino-2-fluorophenyl)(methyl)(oxo)-λ⁶-sulfaneylidene)carbamate (138 mg, 0.48 mmol), HATU (273 mg, 0.72 mmol) and DIEA (155 mg, 1.20 mmol) in DMF (5 mL) was stirred at room temperature for 16 hours. After the reaction was completed, the resulting solution was diluted with water (30 mL) and extracted with EtOAc (20 mL×3). The combined organic phases were washed with brine, dried over Na₂SO₄, concentrated under vacuum. The residue was purified by flash column chromatography on silica gel (PE/EtOAc=2/1 to 1/2) to give tert-butyl (S)-((5-(5-chloro-4-(trifluoromethyl)-2-(4-(trifluoromethyl)piperidin-1-yl)benzamido)-2-fluorophenyl)(methyl)(oxo)-λ⁶-sulfaneylidene)carbamate (200 mg, 69.8%) as a yellow solid. LCMS (ESI) calcd. for C₂₆H₂₈ClF₇N₃O₄S [M+H]⁺ m/z 646.14, found 646.1.

Step 2: (S)-5-chloro-N-(4-fluoro-3-(S-methylsulfonimidoyl)phenyl)-4-(trifluoromethyl)-2-(4-(trifluoromethyl)piperidin-1-yl)benzamide: A solution of tert-butyl (S)-((5-(5-chloro-4-(trifluoromethyl)-2-(4-(trifluoromethyl)piperidin-1-yl)benzamido)-2-fluorophenyl)(methyl)(oxo)-λ⁶-sulfaneylidene)carbamate (200 mg, 0.37 mmol) in DCM (5 mL) was added TFA (0.5 mL) at room temperature. The reaction mixture was stirred at room temperature for 3 hours. After the reaction was completed, the mixture was concentrated. The residue was adjusted to pH=8-9 with saturated aqueous NaHCO₃. Then the aqueous solution was extracted with DCM (15 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.05% NH₃) to provide (S)-5-chloro-N-(4-fluoro-3-(S-methylsulfonimidoyl)phenyl)-4-(trifluoromethyl)-2-(4-(trifluoromethyl)piperidin-1-yl)benzamide (65 mg, 32.2%) as a white solid. ¹H NMR (400 MHz, DMSO-d₆) δ 10.88 (s, 1H), 8.38 (dd, J=6.5, 2.7 Hz, 1H), 7.97-7.84 (m, 1H), 7.80 (s, 1H), 7.55-7.25 (m, 2H), 4.67 (s, 1H), 3.40-3.36 (m, 2H), 3.19 (s, 3H), 2.92-2.87 (m, 2H), 2.47-2.37 (m, 1H), 1.87-1.83 (m, 2H), 1.56-1.43 (m, 2H). LCMS (ESI) calcd. for C₂₁H₂₀ClF₇N₃O₂S [M+H]⁺ m/z 546.08, found 546.0.

### Example 247

### (S)-5-chloro-2-(4,4-difluoropiperidin-1-yl)-N-(2-(S-methylsulfonimidoyl)pyridin-4-yl)-4-(trifluoromethyl)benzamide

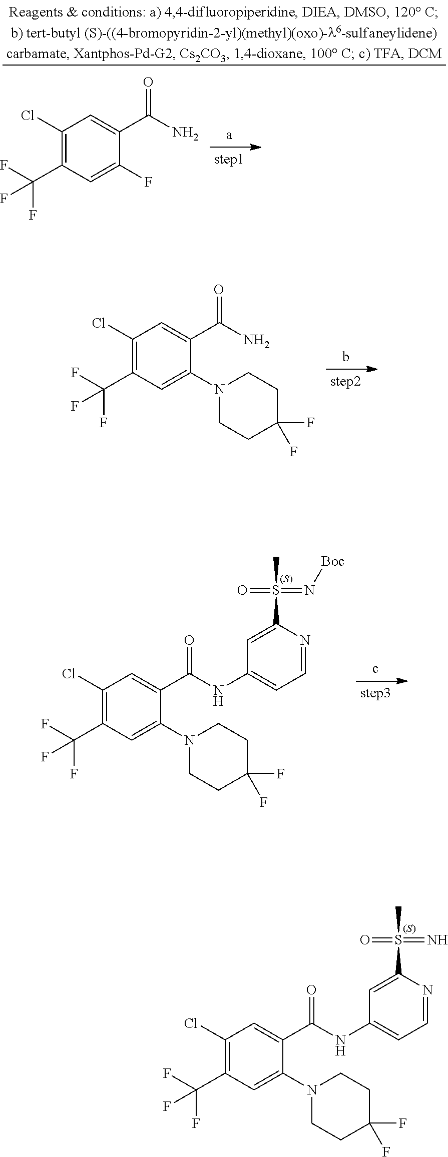

Step 1: 5-chloro-2-(4,4-difluoropiperidin-1-yl)-4-(trifluoromethyl)benzamide: A solution of 5-chloro-2-fluoro-4-(trifluoromethyl)benzamide (3 g, 12.4 mmol), 4,4-difluoropiperidine (1.65 g, 13.7 mmol) and DIEA (4.01 g, 31.0 mmol) in DMSO (50 mL) was heated to 120° C. and stirred for 16 hours. After the reaction was completed, the mixture was cooled to room temperature. The resulting solution was diluted with water (200 mL) and extracted with EtOAc (200 mL×3). The combined organic layers were washed with brine, dried over sodium sulfate, concentrated under vacuum. The residue was purified by flash column chromatography on silica gel (PE/EtOAc=1/1 to 1/2) to provide 5-chloro-2-(4,4-difluoropiperidin-1-yl)-4-(trifluoromethyl)benzamide (3.0 g, 70.42%) as a yellow solid. LCMS (ESI) calcd. for C₁₃H₁₃ClF₅N₂O [M+H]⁺ m/z 343.06, found 343.1.

Step 2: tert-butyl (S)-((4-(5-chloro-2-(4,4-difluoropiperidin-1-yl)-4-(trifluoromethyl)benzamido)pyridin-2-yl)(methyl)(oxo)-λ⁶-sulfaneylidene)carbamate: A mixture of 5-chloro-2-(4,4-difluoropiperidin-1-yl)-4-(trifluoromethyl)benzamide (200 mg, 0.58 mmol), tert-butyl (S)-((4-bromopyridin-2-yl)(methyl)(oxo)-λ⁶-sulfaneylidene)carbamate (206 mg, 0.61 mmol), Cs₂CO₃ (494 mg, 1.52 mmol) and Xantphos-Pd-G2 (104 mg, 0.12 mmol) in 1,4-dioxane (5 mL) was heated to 100° C. and stirred for 2 hours under a nitrogen atmosphere. After the reaction was completed, the mixture was filtered through celite. The filtrate was concentrated and residue was purified by flash column chromatography on silica gel (PE/EtOAc=2/1 to 1/1) to provide tert-butyl (S)-((4-(5-chloro-2-(4,4-difluoropiperidin-1-yl)-4-(trifluoromethyl)benzamido)pyridin-2-yl)(methyl)(oxo)-λ⁶-sulfaneylidene)carbamate (200 mg, 57.30%) as a yellow solid. LCMS (ESI) calcd. for C₂₄H₂₇ClF₅N₄O₄S [M+H]⁺ m/z 597.14, found 597.1.

Step 3: (S)-5-chloro-2-(4,4-difluoropiperidin-1-yl)-N-(2-(S-methylsulfonimidoyl)pyridin-4-yl)-4-(trifluoromethyl)benzamide: To a solution of tert-butyl (S)-((4-(5-chloro-2-(4,4-difluoropiperidin-1-yl)-4-(trifluoromethyl)benzamido)pyridin-2-yl)(methyl)(oxo)-λ⁶-sulfaneylidene)carbamate (200 mg, 0.33 mmol) in DCM (5 mL) was added TFA (0.5 mL) at room temperature. The reaction mixture was stirred at room temperature for 1 hour. After the reaction was completed, the mixture was concentrated. The residue was adjusted to pH=8-9 with saturated aqueous NaHCO₃. Then the aqueous solution was extracted with DCM (15 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.05% NH₃) to provide (S)-5-chloro-2-(4,4-difluoropiperidin-1-yl)-N-(2-(S-methylsulfonimidoyl)pyridin-4-yl)-4-(trifluoromethyl)benzamide (102 mg, 64.21%) as a white solid. ¹H NMR (400 MHz, DMSO-d₆) δ 11.32 (s, 1H), 8.69 (s, 1H), 8.56 (s, 1H), 7.85 (s, 2H), 7.56 (s, 1H), 3.36 (s, 3H), 3.20 (d, J=5.0 Hz, 4H), 2.04-1.92 (m, 4H). LCMS (ESI) calcd. for C₁₉H₁₉ClF₅N₄O₂S [M+H]⁺ m/z 497.08, found 497.1.

### Example 248

### (R)-5-chloro-2-(4,4-difluoropiperidin-1-yl)-N-(2-(S-methylsulfonimidoyl)pyridin-4-yl)-4-(trifluoromethyl)benzamide

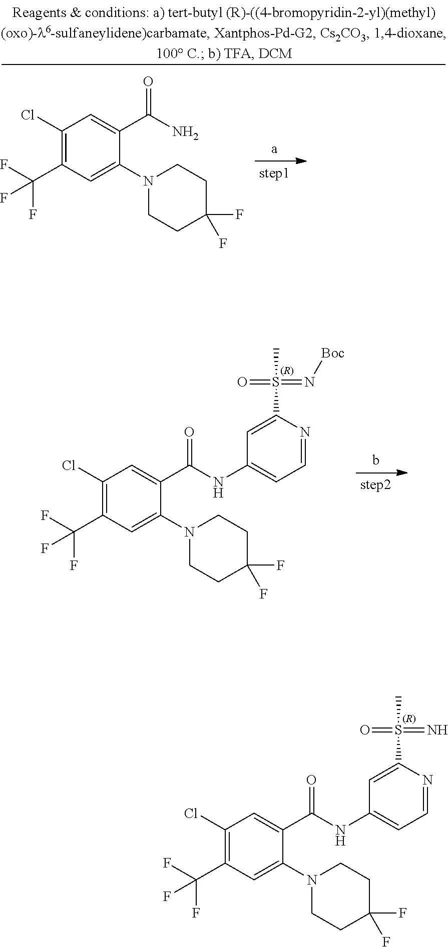

Step 1: tert-butyl (R)-((4-(5-chloro-2-(4,4-difluoropiperidin-1-yl)-4-(trifluoromethyl)benzamido)pyridin-2-yl)(methyl)(oxo)-λ⁶-sulfaneylidene)carbamate: A mixture of 5-chloro-2-(4,4-difluoropiperidin-1-yl)-4-(trifluoromethyl)benzamide (200 mg, 0.58 mmol), tert-butyl (R)-((4-bromopyridin-2-yl)(methyl)(oxo)-λ⁶-sulfaneylidene)carbamate (206 mg, 0.61 mmol), Cs₂CO₃ (494 mg, 1.52 mmol) and Xantphos-Pd-G2 (104 mg, 0.12 mmol) in 1,4-dioxane (5 mL) was heated to 100° C. and stirred for 2 hours under a nitrogen atmosphere. After the reaction was completed, the mixture was filtered through celite. The filtrate was concentrated and residue was purified by flash column chromatography on silica gel (PE/EtOAc=1/1) to provide tert-butyl (R)-((4-(5-chloro-2-(4,4-difluoropiperidin-1-yl)-4-(trifluoromethyl)benzamido)pyridin-2-yl)(methyl)(oxo)-λ⁶-sulfaneylidene)carbamate (200 mg, 57.30%) as a yellow solid. LCMS (ESI) calcd. for C₂₄H₂₇ClF₅N₄O₄S [M+H]⁺ m/z 597.14, found 597.1.

Step 2: (R)-5-chloro-2-(4,4-difluoropiperidin-1-yl)-N-(2-(S-methylsulfonimidoyl)pyridin-4-yl)-4-(trifluoromethyl)benzamide: A solution of tert-butyl (R)-((4-(5-chloro-2-(4,4-difluoropiperidin-1-yl)-4-(trifluoromethyl)benzamido)pyridin-2-yl)(methyl)(oxo)-λ⁶-sulfaneylidene)carbamate (200 mg, 0.33 mmol) in DCM (5 mL) was added TFA (0.5 mL) at room temperature. The reaction mixture was stirred at room temperature for 1 hour. After the reaction was completed, the mixture was concentrated. The residue was adjusted to pH=8-9 with saturated aqueous NaHCO₃. Then the aqueous solution was extracted with DCM (15 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.05% NH₃) to provide (R)-5-chloro-2-(4,4-difluoropiperidin-1-yl)-N-(2-(S-methylsulfonimidoyl)pyridin-4-yl)-4-(trifluoromethyl)benzamide (95 mg, 58.77%) as a white solid. ¹H NMR (400 MHz, DMSO-d₆) δ 11.32 (s, 1H), 8.70 (d, J=5.4 Hz, 1H), 8.56 (s, 1H), 7.85 (s, 2H), 7.56 (s, 1H), 3.36 (s, 3H), 3.28-3.14 (m, 4H), 2.07-1.91 (m, 4H). LCMS (ESI) calcd. for C₁₉H₁₉ClF₅N₄O₂S [M+H]⁺ m/z 497.08, found 497.1.

### Example 249

### (S)-5-chloro-2-(4,4-difluoropiperidin-1-yl)-N-(4-fluoro-3-(S-methylsulfonimidoyl)phenyl)-4-(trifluoromethyl)benzamide

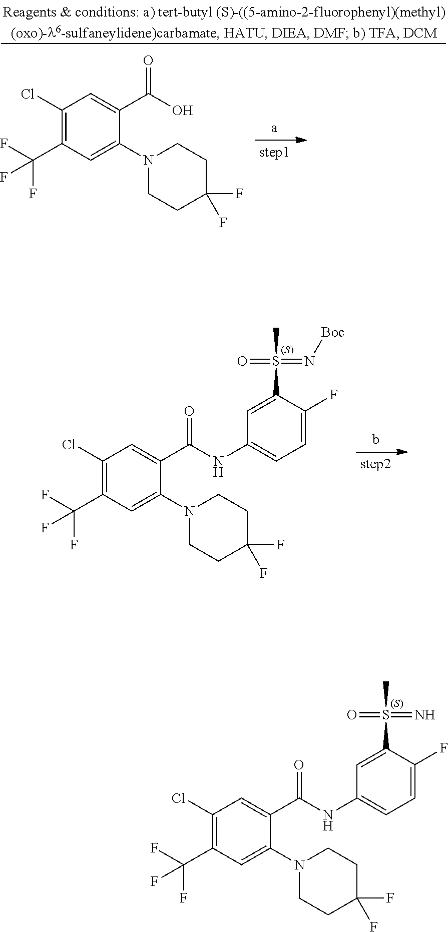

Step 1: tert-butyl (S)-((5-(5-chloro-2-(4,4-difluoropiperidin-1-yl)-4-(trifluoromethyl)benzamido)-2-fluorophenyl)(methyl)(oxo)-λ⁶-sulfaneylidene)carbamate: To a solution of 5-chloro-2-(4,4-difluoropiperidin-1-yl)-4-(trifluoromethyl)benzoic acid (100 mg, 0.29 mmol), tert-butyl (S)-((5-amino-2-fluorophenyl)(methyl)(oxo)-λ⁶-sulfaneylidene)carbamate (126 mg, 0.44 mmol), HATU (166 mg, 0.44 mmol) and DIEA (113 mg, 0.87 mmol) in DMF (5 mL) stirred 25° C. for 16 h. After the reaction was completed, the mixture was diluted with water and extracted with EtOAc (50 mL×3). The combined organic layers were washed with brine, dried over Na₂SO₄, and concentrated under vacuum. The residue was purified by flash column chromatography on silica gel (PE/EtOAc=1/1) to give tert-butyl (S)-((5-(5-chloro-2-(4,4-difluoropiperidin-1-yl)-4-(trifluoromethyl)benzamido)-2-fluorophenyl)(methyl)(oxo)-λ⁶-sulfaneylidene)carbamate (100 mg, 55.9%) as yellow solid. LCMS (ESI) calcd. for C₂₅H₂₆ClF₆N₃NaO₄S [M+Na]⁺ m/z 636.11, found 636.0.

Step 2: (S)-5-chloro-2-(4,4-difluoropiperidin-1-yl)-N-(4-fluoro-3-(S-methylsulfonimidoyl)phenyl)-4-(trifluoromethyl)benzamide: A solution of tert-butyl (S)-((5-(5-chloro-2-(4,4-difluoropiperidin-1-yl)-4-(trifluoromethyl)benzamido)-2-fluorophenyl)(methyl)(oxo)-λ⁶-sulfaneylidene)carbamate (100 mg, 0.16 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (15 mL×3). The combined organic layers were washed with brine, dried over sodium sulfate, concentrated under vacuum. The residue was purified by prep-HPLC (Gemini 5 um C₁₈ column, 150*21.2 mm, eluting with 60% to 95% MeCN/H₂O containing 0.05% NH₃) to provide (S)-5-chloro-2-(4,4-difluoropiperidin-1-yl)-N-(4-fluoro-3-(S-methylsulfonimidoyl)phenyl)-4-(trifluoromethyl)benzamide (25.9 mg, 31.5%) as a white solid. ¹H NMR (400 MHz, DMSO-d₆) δ 10.86 (s, 1H), 8.41-8.29 (m, 1H), 7.98-7.88 (m, 1H), 7.80 (s, 1H), 7.56-7.41 (m, 2H), 4.70 (s, 1H), 3.26-3.16 (m, 7H), 2.10-1.93 (m, 4H). LCMS (ESI) calcd. for C₂₀H₁₉ClF₆N₃O₂S [M+H]⁺ m/z 514.08, found 514.0.

### Example 250

### (R)-4-cyclopropyl-6-(4,4-difluoroazepan-1-yl)-2-methyl-N-(2-(S-methylsulfonimidoyl)pyridin-4-yl)-3-(trifluoromethyl)benzamide

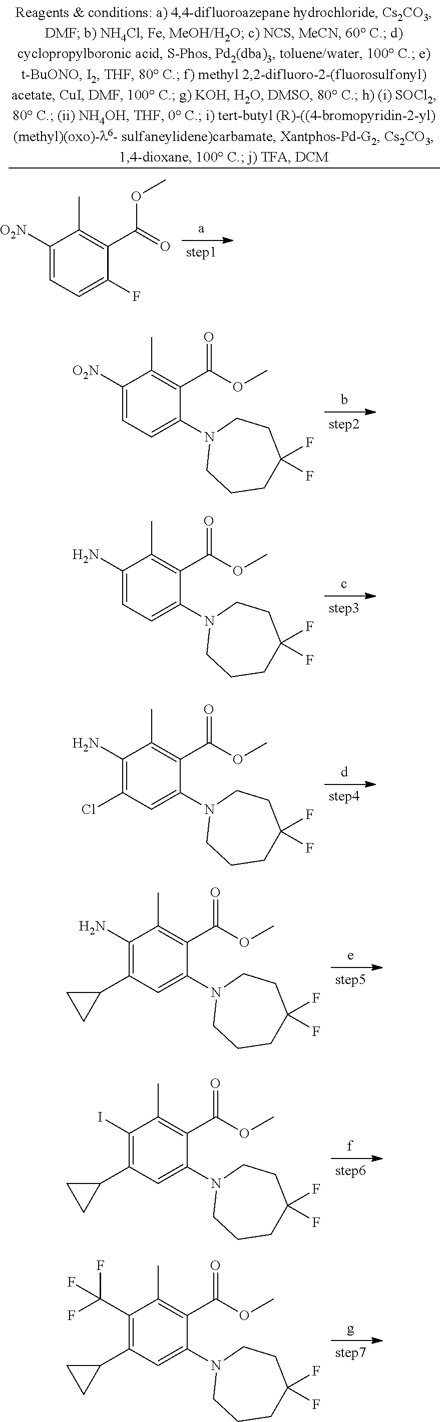

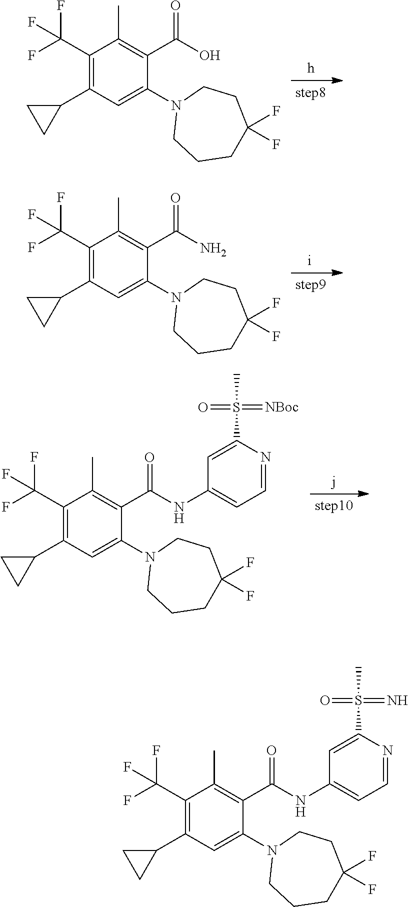

Step 1: methyl 6-(4,4-difluoroazepan-1-yl)-2-methyl-3-nitrobenzoate. A solution of methyl 6-fluoro-2-methyl-3-nitrobenzoate (8 g, 37.5 mmol), 4,4-difluoroazepane hydrochloride (7.08 g, 41.3 mmol), and cesium carbonate (36.65 g, 112.5 mmol) in DMF (100 mL) was heated to 50° C. and stirred for 16 hours. After the reaction was completed, the mixture was cooled to room temperature. The resulting solution was diluted with water (100 mL) and extracted with EtOAc (100 mL×3). The combined organic layers were washed with brine, dried over sodium sulfate, concentrated under vacuum. The residue was purified by flash column chromatography on silica gel (PE/EtOAc=4/1) to provide methyl 6-(4,4-difluoroazepan-1-yl)-2-methyl-3-nitrobenzoate (7.5 g, 60.80%) as yellow solid. LCMS (ESI) calcd. for C₁₅H₁₉F₂N₂O₄ [M+H]⁺ m/z 329.12, found 329.1.

Step 2: methyl 3-amino-6-(4,4-difluoroazepan-1-yl)-2-methylbenzoate: A solution of methyl 6-(4,4-difluoroazepan-1-yl)-2-methyl-3-nitrobenzoate (7.9 g, 24.1 mmol) in MeOH (90 mL) and water (30 mL) was added NH₄Cl (6.45 g, 120.5 mmol), Fe (6.06 g, 108.5 mmol). The mixture was heated at 60° C. for 5 hours. After the reaction was completed. The mixture was filtered through celite. The filtrate was diluted with water (100 mL) and extracted with EtOAc (100 mL×3). The combined organic layers were washed with brine, dried over sodium sulfate, concentrated under vacuum to provide methyl 3-amino-6-(4,4-difluoroazepan-1-yl)-2-methylbenzoate (6 g, 83.40%) as a yellow solid which was used directly in next step without further purification. LCMS (ESI) calcd. For C₁₅H₂₁F₂N₂O₂ [M+H]⁺ m/z 299.15, found 299.1.

Step 3: methyl 3-amino-4-chloro-6-(4,4-difluoroazepan-1-yl)-2-methylbenzoate: A solution of methyl 3-amino-6-(4,4-difluoroazepan-1-yl)-2-methylbenzoate (3.7 g, 12.4 mmol) and NCS (1.99 g, 14.9 mmol) in MeCN (80 mL) was heated to 60° C. and stirred for 6 hours. After the reaction was completed, the mixture was cooled to room temperature. The resulting solution was diluted with water (100 mL) and extracted with EtOAc (100 mL×3). The combined organic layers were washed with brine, dried over sodium sulfate, concentrated under vacuum. The residue was purified by flash column chromatography on silica gel (PE/EtOAc=2/1) to provide methyl 3-amino-4-chloro-6-(4,4-difluoroazepan-1-yl)-2-methylbenzoate (1.5 g, 36.29%) as yellow oil. LCMS (ESI) calcd. for C₁₅H₂₀ClF₂N₂O₂ [M+H]⁺ m/z 333.11, found 333.0.

Step 4: methyl 3-amino-4-cyclopropyl-6-(4,4-difluoroazepan-1-yl)-2-methylbenzoate: To a solution of methyl 3-amino-4-chloro-6-(4,4-difluoroazepan-1-yl)-2-methylbenzoate (1.5 g, 4.51 mmol) and cyclopropylboronic acid (387 mg, 4.51 mmol) in toluene (20 mL) was added K₃PO₄ (1.91 g, 9.02 mmol), S-Phos (185 mg, 0.45 mmol), Pd₂(dba)₃ (413 mg, 0.45 mmol) and H₂O (5 mL) at room temperature. The mixture was heated at 100° C. for 16 h under an atmosphere of N₂. After the reaction was completed, the mixture was quenched with water (50 mL) and extracted with EtOAc (50 mL×3). The combined organic layers were washed with brine, dried over sodium sulfate, concentrated under vacuum. The residue was purified by flash column chromatography on silica gel (PE/EtOAc=3/1) to provide methyl 3-amino-4-cyclopropyl-6-(4,4-difluoroazepan-1-yl)-2-methylbenzoate (1 g, 65.56%) as yellow oil. LCMS (ESI) calcd. for C₁₈H₂₅F₂N₂O₂ [M+H]⁺ m/z 339.18, found 339.2.

Step 5: methyl 4-cyclopropyl-6-(4,4-difluoroazepan-1-yl)-3-iodo-2-methylbenzoate: To a solution of methyl 3-amino-4-cyclopropyl-6-(4,4-difluoroazepan-1-yl)-2-methylbenzoate (620 mg, 1.83 mmol) in THE (10 mL) was added I₂ (698 mg, 2.75 mmol. The reaction solution was stirred at room temperature for 0.5 h. Then t-BuONO (283 mg, 2.75 mmol) was added, and the reaction solution was heated at 80° C. for 1 hour. After the reaction was completed, the mixture was quenched with water (50 mL) and extracted with EtOAc (50 mL×3). The combined organic layers were washed with brine, dried over sodium sulfate, concentrated under vacuum. The residue was purified by flash column chromatography on silica gel (PE/EtOAc=10/1) to provide methyl 4-cyclopropyl-6-(4,4-difluoroazepan-1-yl)-3-iodo-2-methylbenzoate (300 mg, 36.44% yield) as yellow oil. LCMS (ESI) calcd. for C₁₈H₂₃F₂₁NO₂ [M+H]⁺ m/z 450.07, found 450.0.

Step 6: methyl 4-cyclopropyl-6-(4,4-difluoroazepan-1-yl)-2-methyl-3-(trifluoromethyl)benzoate: To a solution of methyl 4-cyclopropyl-6-(4,4-difluoroazepan-1-yl)-3-iodo-2-methylbenzoate (400 mg, 0.89 mmol) and CuI (339 mg, 1.78 mmol) in DMF (10 mL) was added methyl 2,2-difluoro-2-(fluorosulfonyl)acetate (2.05 g, 10.68 mmol) dropwise at room temperature under an atmosphere of N₂. The mixture was heated at 100° C. for 2 hours. After the reaction was completed, the resulting solution was diluted with water (50 mL) and extracted with EtOAc (50 mL×3). The combined organic phases were washed with brine, dried over sodium sulfate, concentrated under vacuum. The residue was purified by flash column chromatography on silica gel (PE/EtOAc=10/1) to give methyl 4-cyclopropyl-6-(4,4-difluoroazepan-1-yl)-2-methyl-3-(trifluoromethyl)benzoate (245 mg, 70.31%). LCMS (ESI) calcd. for C₁₉H₂₃F₅NO₂ [M+H]⁺ m/z 392.16, found 392.1.

Step 7: 4-cyclopropyl-6-(4,4-difluoroazepan-1-yl)-2-methyl-3-(trifluoromethyl)benzoic acid: To a solution of methyl 4-cyclopropyl-6-(4,4-difluoroazepan-1-yl)-2-methyl-3-(trifluoromethyl)benzoate (100 mg, 0.26 mmol) in DMSO/H₂O (1/1, 10 mL) was added KOH (143 mg, 2.56 mmol) at room temperature. The mixture was stirred at 80° C. for 3 hours. After the reaction was completed. The aqueous phase was adjusted to pH=3-4 with 1N HCl then extracted with EtOAc (30 mL×3). The combined organic layers were washed with brine, dried with Na₂SO₄, concentrated under reduced pressure to give 4-cyclopropyl-6-(4,4-difluoroazepan-1-yl)-2-methyl-3-(trifluoromethyl)benzoic acid (80 mg, 82.97%) as a yellow solid. LCMS (ESI) calcd. for C₁₈H₂₁F₅NO₂ [M+H]⁺ m/z 378.14, found 378.2.

Step 8: 4-cyclopropyl-6-(4,4-difluoroazepan-1-yl)-2-methyl-3-(trifluoromethyl)benzamide: A solution of 4-cyclopropyl-6-(4,4-difluoroazepan-1-yl)-2-methyl-3-(trifluoromethyl)benzoic acid (80 mg, 0.21 mmol) in SOCl₂ (5 mL) was heated to 80° C. and stirred for 0.5 hour. The solution was concentrated under vacuum to provide the chloride intermediate. Then the chloride intermediate dissolved in THE (5 mL) was added to a solution of THF (5 mL) in NH₃·H₂O (5 mL) and at 0° C. The resulting mixture was stirred at 25° C. for 1 hour. Then the mixture was quenched with water (20 mL) and extracted with DCM (20 mL×3). The combined organic layers were washed with brine, dried over sodium sulfate, concentrated under vacuum. The residue was purified by flash column chromatography on silica gel (PE/EtOAc=1/1) to provide 4-cyclopropyl-6-(4,4-difluoroazepan-1-yl)-2-methyl-3-(trifluoromethyl)benzamide (70 mg, 87.74%) as yellow oil. LCMS (ESI) calcd. for C₁₈H₂₂F₅N₂O [M+H]⁺ m/z 377.16, found 377.2.

Step 9: tert-butyl (R)-((4-(4-cyclopropyl-6-(4,4-difluoroazepan-1-yl)-2-methyl-3-(trifluoromethyl)benzamido)pyridin-2-yl)(methyl)(oxo)-λ⁶-sulfaneylidene)carbamate: A mixture of 4-cyclopropyl-6-(4,4-difluoroazepan-1-yl)-2-methyl-3-(trifluoromethyl)benzamide (70 mg, 0.19 mmol), tert-butyl (R)-((4-bromopyridin-2-yl)(methyl)(oxo)-λ⁶-sulfaneylidene)carbamate (66 mg, 0.20 mmol), Cs₂CO₃ (158 mg, 0.48 mmol) and Xantphos-Pd-G2 (33 mg, 0.04 mmol) in 1,4-dioxane (5 mL) was stirred at 100° C. for 2 hours under nitrogen. After the reaction was completed, the mixture was filtered through celite. The filtrate was concentrated and residue was purified by flash column chromatography on silica gel (PE/EtOAc=1/1) to provide tert-butyl (R)-((4-(4-cyclopropyl-6-(4,4-difluoroazepan-1-yl)-2-methyl-3-(trifluoromethyl)benzamido)pyridin-2-yl)(methyl)(oxo)-λ⁶-sulfaneylidene)carbamate (100 mg, 85.11%) as a yellow solid. LCMS (ESI) calcd. for C₂₉H₃₆F₅N₄O₄S [M+H]⁺ m/z 631.23, found 631.3.

Step 10: (R)-4-cyclopropyl-6-(4,4-difluoroazepan-1-yl)-2-methyl-N-(2-(S-methylsulfonimidoyl)pyridin-4-yl)-3-(trifluoromethyl)benzamide: A solution of tert-butyl (R)-((4-(4-cyclopropyl-6-(4,4-difluoroazepan-1-yl)-2-methyl-3-(trifluoromethyl)benzamido)pyridin-2-yl)(methyl)(oxo)-λ⁶-sulfaneylidene)carbamate (100 mg, 0.16 mmol) in DCM (5 mL) was added TFA (0.5 mL) at room temperature. The reaction mixture was stirred at room temperature for 1 hour. After the reaction was completed, the mixture was concentrated. The residue was adjusted to pH=8-9 with saturated aqueous NaHCO₃. Then the aqueous solution was extracted with DCM (15 mL×3). The combined organic layers were washed with brine, dried over sodium sulfate, concentrated under vacuum. The residue was purified by prep-IPLC (Gemini 5 um C₁₈ column, 150*21.2 mm, eluting with 50% to 95% MeCN/H₂O containing 0.05% NH₃) to provide (R)-4-cyclopropyl-6-(4,4-difluoroazepan-1-yl)-2-methyl-N-(2-(S-methylsulfonimidoyl)pyridin-4-yl)-3-(trifluoromethyl)benzamide (20 mg, 23.82%) as a white solid. ¹H NMR (400 MHz, DMSO-d₆) δ 11.17 (s, 1H), 8.60 (d, J=5.3 Hz, 1H), 8.43 (s, 1H), 7.80 (d, J=4.1 Hz, 1H), 6.85 (s, 1H), 4.35 (s, 1H), 3.30-3.18 (m, 4H), 3.13 (s, 3H), 2.32 (d, J=2.9 Hz, 3H), 2.22-1.92 (m, 5H), 1.75-1.61 (m, 2H), 0.97 (d, J=7.4 Hz, 2H), 0.73 (d, J=4.9 Hz, 2H). LCMS (ESI) calcd. for C₂₄H₂₈F₅N₄O₂S [M+H]⁺ m/z 531.18, found 531.2.

### Example 251

### (S)-5-chloro-N-(3-(S-methylsulfonimidoyl)phenyl)-4-(trifluoromethyl)-2-(4-(trifluoromethyl)piperidin-1-yl)benzamide

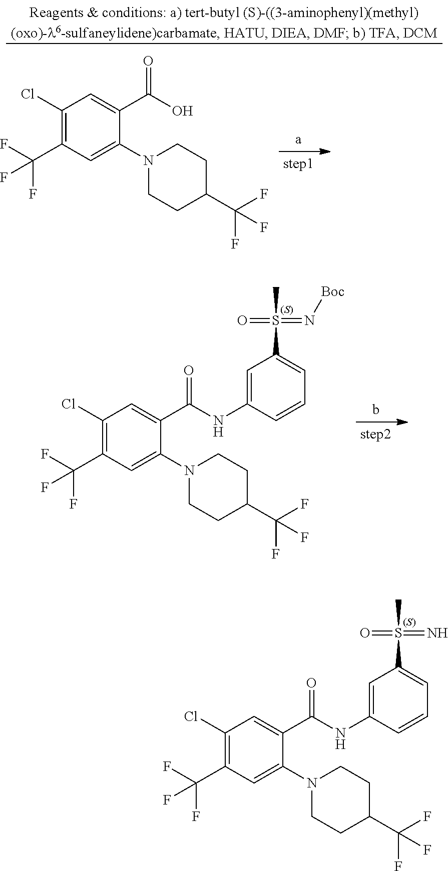

Step 1: tert-butyl (S)-((3-(5-chloro-4-(trifluoromethyl)-2-(4-(trifluoromethyl)piperidin-1-yl)benzamido)phenyl)(methyl)(oxo)-λ⁶-sulfaneylidene)carbamate: A mixture of 5-chloro-4-(trifluoromethyl)-2-(4-(trifluoromethyl)piperidin-1-yl)benzoic acid (150 mg, 0.40 mmol), tert-butyl (S)-((3-aminophenyl)(methyl)(oxo)-λ⁶-sulfaneylidene)carbamate (130 mg, 0.48 mmol), HATU (273 mg, 0.72 mmol) and DIEA (155 mg, 1.20 mmol) in DMF (5 mL) was stirred at room temperature for 16 hours. After the reaction was completed, the resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtOAc=2/1 to 1/1) to give tert-butyl (S)-((3-(5-chloro-4-(trifluoromethyl)-2-(4-(trifluoromethyl)piperidin-1-yl)benzamido)phenyl)(methyl)(oxo)-λ⁶-sulfaneylidene)carbamate (180 mg, 64.5%) as a yellow solid. LCMS (ESI) calcd. for C₂₆H₂₉ClF₆N₃O₄S [M+H]⁺ m/z 628.15, found 628.00.

Step 2: (S)-5-chloro-N-(3-(S-methylsulfonimidoyl)phenyl)-4-(trifluoromethyl)-2-(4-(trifluoromethyl)piperidin-1-yl)benzamide: A solution of tert-butyl (S)-((3-(5-chloro-4-(trifluoromethyl)-2-(4-(trifluoromethyl)piperidin-1-yl)benzamido)phenyl)(methyl)(oxo)-λ⁶-sulfaneylidene)carbamate (180 mg, 0.29 mmol) in DCM (5 mL) was added TFA (0.5 mL) at room temperature. The reaction mixture was stirred at room temperature for 3 hours. After the reaction was completed, the mixture was concentrated. The residue was adjusted to pH=8-9 with saturated aqueous NaHCO₃. Then the aqueous solution was extracted with DCM (15 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.05% NH₃) to provide (S)-5-chloro-N-(3-(S-methylsulfonimidoyl)phenyl)-4-(trifluoromethyl)-2-(4-(trifluoromethyl)piperidin-1-yl)benzamide (70 mg, 45.8%) as a white solid. ¹H NMR (400 MHz, DMSO-d₆) δ 10.92 (s, 1H), 8.42 (s, 1H), 7.90 (d, J=8.1 Hz, 1H), 7.82 (s, 1H), 7.68 (d, J=7.8 Hz, 1H), 7.61 (t, J=7.9 Hz, 1H), 7.48 (s, 1H), 4.21 (s, 1H), 3.40-3.36 (m, 2H), 3.05 (s, 3H), 2.93-2.87 (m, 2H), 2.47-2.37 (m, 1H), 1.88-1.82 (m, 2H), 1.58-1.44 (m, 2H). LCMS (ESI) calcd. for C₂₁H₂₁ClF₆N₃O₂S [M+H]⁺ m/z 528.09, found

### Example 252

### N-((trans)-3-carbamoylcyclobutyl)-5-chloro-2-(4,4-difluoroazepan-1-yl)-4-(trifluoromethyl)benzamide

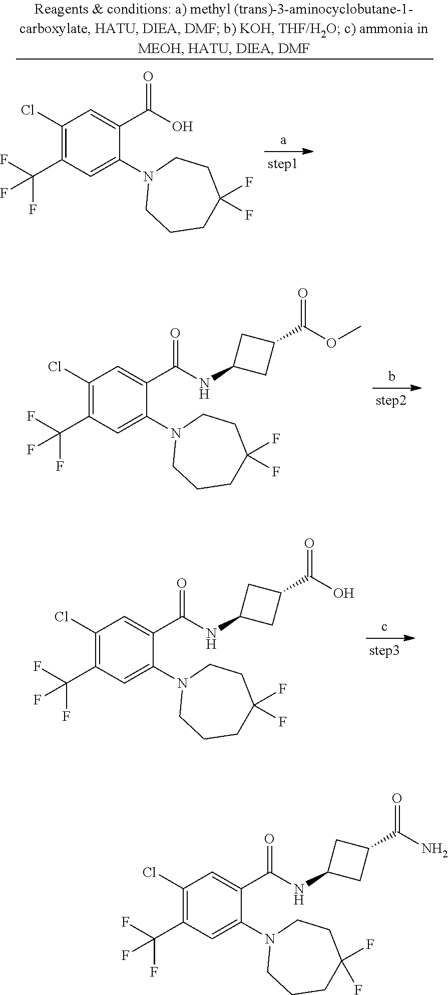 [SMILES: O=C(O)c1cc(Cl)c(C(F)(F)F)cc1N1CCCC(F)(F)CC1 ; COC(=O)[C@H]1C[C@H](NC(=O)c2cc(Cl)c(C(F)(F)F)cc2N2CCCC(F)(F)CC2)C1 ; O=C(N[C@H]1C[C@H](C(=O)O)C1)c1cc(Cl)c(C(F)(F)F)cc1N1CCCC(F)(F)CC1 ; NC(=O)[C@H]1C[C@H](NC(=O)c2cc(Cl)c(C(F)(F)F)cc2N2CCCC(F)(F)CC2)C1]

Step 1: methyl (trans)-3-(5-chloro-2-(4,4-difluoroazepan-1-yl)-4-(trifluoromethyl)benzamido)cyclobutane-1-carboxylate: A mixture of 5-chloro-2-(4,4-difluoroazepan-1-yl)-4-(trifluoromethyl)benzoic acid (200 mg, 0.56 mmol) and methyl (trans)-3-aminocyclobutane-1-carboxylate (110 mg, 0.67 mmol) in DMF (5 mL) was added HATU (277 mg, 0.73 mmol) and DIEA (232 mg, 1.80 mmol). The mixture was stirred at room temperature for 4 hours. The reaction was monitored by LCMS. After the reaction was completed, the resulting solution was diluted with water (15 mL) and extracted with DCM (15 mL×3). The combined organic layers were washed with brine, dried over Na₂SO₄, concentrated under vacuum. The residue was purified by flash column chromatography on silica gel (PE/EtOAc=2/1 to 1/1) to give methyl (trans)-3-(5-chloro-2-(4,4-difluoroazepan-1-yl)-4-(trifluoromethyl)benzamido)cyclobutane-1-carboxylate (210 mg, 79.85%) as white oil. LCMS (ESI) calcd. for C₂₀H₂₃ClF₅N₂O₃ [M+H]⁺ m/z 469.13, found 469.0.

Step 2: (trans)-3-(5-chloro-2-(4,4-difluoroazepan-1-yl)-4-(trifluoromethyl)benzamido)cyclobutane-1-carboxylic acid: A solution of methyl (trans)-3-(5-chloro-2-(4,4-difluoroazepan-1-yl)-4-(trifluoromethyl)benzamido)cyclobutane-1-carboxylate 2 (210 mg, 0.45 mmol) in THF/H₂O (1:1, 10 mL) was added KOH (239 mg, 4.50 mmol) at room temperature. The mixture was stirred at room temperature for 2 hours. The reaction was monitored by LCMS. After the reaction was completed, the reaction was extracted with DCM (20 mL), then the aqueous phase was adjusted to pH=3 with 1 N HCl and extracted with DCM (20 mL×3). The organic phase was dried with Na₂SO₄ and concentrated under vacuum to give (trans)-3-(5-chloro-2-(4,4-difluoroazepan-1-yl)-4-(trifluoromethyl)benzamido)cyclobutane-1-carboxylic acid (180 mg, 87.80%) as a white solid. LCMS (ESI) calcd. for C₁₉H₂₁ClF₅N₂O₃ [M+H]⁺ m/z 455.12, found 455.05.

Step 3: N-((trans)-3-carbamoylcyclobutyl)-5-chloro-2-(4,4-difluoroazepan-1-yl)-4-(trifluoromethyl)benzamide: A solution of (trans)-3-(5-chloro-2-(4,4-difluoroazepan-1-yl)-4-(trifluoromethyl)benzamido)cyclobutane-1-carboxylic acid (180 mg, 0.40 mmol) in DMF (5 mL) was added HATU (218 mg, 0.51 mmol), DIEA (183 mg, 1.28 mmol) and NH₃-MeOH (5 mL) at room temperature. The reaction mixture was stirred at room temperature for 2 hours. After the reaction was completed, the solution was diluted with water (10 mL) and extracted with DCM (10 mL×3). The combined organic layers were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.1% formic acid) to obtain N-((trans)-3-carbamoylcyclobutyl)-5-chloro-2-(4,4-difluoroazepan-1-yl)-4-(trifluoromethyl)benzamide (39.20 mg, 22.22%) as a white solid. ¹H NMR (400 MHz, DMSO-d₆, ppm) δ 8.84 (d, J=7.5 Hz, 1H), 7.45 (s, 1H), 7.26 (s, 2H), 6.79 (s, 1H), 4.49-4.43 (m, 1H), 3.37-3.33 (m, 2H), 3.30 (s, 3H), 2.90-2.83 (m, 1H), 2.41-2.35 (m, 2H), 2.26-2.08 (m, 6H), 1.85-1.79 (m, 2H). LCMS (ESI) calcd. for C₁₉H₂₂ClF₅N₃O₂ [M+H]⁺ m/z 454.13, found 454.0.

### Example 253

### (S)-4-cyclopropyl-6-(4,4-difluoroazepan-1-yl)-2-methyl-N-(2-(S-methylsulfonimidoyl)pyridin-4-yl)-3-(trifluoromethyl)benzamide

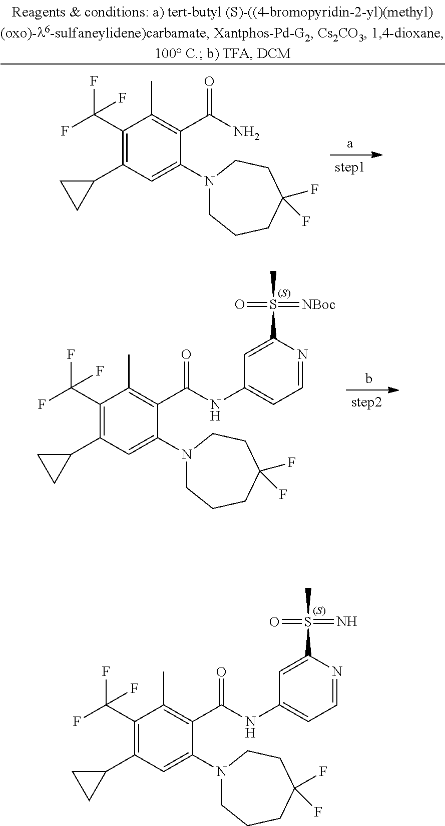

Step 1: tert-butyl (S)-((4-(4-cyclopropyl-6-(4,4-difluoroazepan-1-yl)-2-methyl-3-(trifluoromethyl)benzamido)pyridin-2-yl)(methyl)(oxo)-λ⁶-sulfaneylidene)carbamate: A mixture of 4-cyclopropyl-6-(4,4-difluoroazepan-1-yl)-2-methyl-3-(trifluoromethyl)benzamide (50 mg, 0.13 mmol), tert-butyl (S)-((4-bromopyridin-2-yl)(methyl)(oxo)-λ⁶-sulfaneylidene)carbamate (47 mg, 0.14 mmol), Cs₂CO₃ (113 mg, 0.35 mmol) and Xantphos-Pd-G2 (24 mg, 0.03 mmol) in 1,4-dioxane (5 mL) was heated at 100° C. for 2 hours under nitrogen. After the reaction was completed, the mixture was filtered through celite. The filtrate was concentrated and residue was purified by flash column chromatography on silica gel (PE/EtOAc=1/1) to provide tert-butyl (S)-((4-(4-cyclopropyl-6-(4,4-difluoroazepan-1-yl)-2-methyl-3-(trifluoromethyl)benzamido)pyridin-2-yl)(methyl)(oxo)-λ⁶-sulfaneylidene)carbamate (40 mg, 47.63%) as a yellow solid. LCMS (ESI) calcd. for C₂₉H₃₆F₅N₄O₄S [M+H]⁺ m/z 631.23, found 631.2.

Step 2: (S)-4-cyclopropyl-6-(4,4-difluoroazepan-1-yl)-2-methyl-N-(2-(S-methylsulfonimidoyl)pyridin-4-yl)-3-(trifluoromethyl)benzamide: A solution of tert-butyl (S)-((4-(4-cyclopropyl-6-(4,4-difluoroazepan-1-yl)-2-methyl-3-(trifluoromethyl)benzamido)pyridin-2-yl)(methyl)(oxo)-λ⁶-sulfaneylidene)carbamate (40 mg, 0.06 mmol) in DCM (5 mL) was added TFA (0.5 mL) at room temperature. The reaction mixture was stirred at room temperature for 1 hour. After the reaction was completed, the mixture was concentrated. The residue was adjusted to pH=8-9 with saturated aqueous NaHCO₃. Then the aqueous solution was extracted with DCM (15 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.05% NH₃) to provide (S)-4-cyclopropyl-6-(4,4-difluoroazepan-1-yl)-2-methyl-N-(2-(S-methylsulfonimidoyl)pyridin-4-yl)-3-(trifluoromethyl)benzamide (2.8 mg, 8.37%) as a white solid. ¹H NMR (400 MHz, DMSO-d₆) δ 11.17 (s, 1H), 8.61 (d, J=5.4 Hz, 1H), 8.43 (d, J=1.7 Hz, 1H), 7.81 (dd, J=5.5, 2.0 Hz, 1H), 6.85 (s, 1H), 4.35 (s, 1H), 3.25 (dd, J=12.3, 6.7 Hz, 4H), 3.13 (s, 3H), 2.35-2.30 (m, 3H), 2.19-1.93 (m, 5H), 1.74-1.65 (m, 2H), 0.98 (dd, J=8.5, 1.5 Hz, 2H), 0.73 (d, J=5.0 Hz, 2H). LCMS (ESI) calcd. for C₂₄H₂₈F₅N₄O₂S [M+H]⁺ m/z 531.18, found 531.2.

### Example 254

### (R)-5-chloro-2-(4,4-difluoropiperidin-1-yl)-N-(4-fluoro-3-(S-methylsulfonimidoyl)phenyl)-4-(trifluoromethyl)benzamide

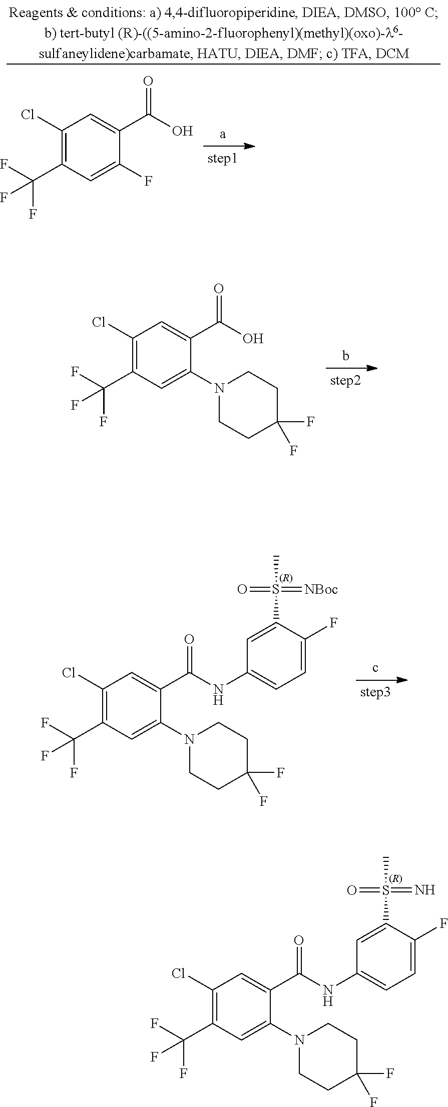

Step 1: 5-chloro-2-(4,4-difluoropiperidin-1-yl)-4-(trifluoromethyl)benzoic acid: A solution of 5-chloro-2-fluoro-4-(trifluoromethyl)benzoic acid (200 mg, 0.82 mmol), 4,4-difluoropiperidine (200 mg, 1.65 mmol) and DIEA (533 mg, 4.12 mmol) in DMSO (5 mL) was heated at 100° C. for 16 hours. After the reaction was completed, the mixture was cooled to room temperature. The resulting solution was diluted with water (30 mL) and extracted with EtOAc (30 mL×3). The combined organic layers were washed with brine, dried over sodium sulfate, concentrated under vacuum. The residue was purified by flash column chromatography on silica gel (PE/EtOAc=1/1) to provide 5-chloro-2-(4,4-difluoropiperidin-1-yl)-4-(trifluoromethyl)benzoic acid (180 mg, 63.5%) as yellow solid. LCMS (ESI) calcd. for C₁₃H₁₂ClF₅NO₂ [M+H]⁺ m/z 344.05, found 344.0.

Step 2: tert-butyl (R)-((5-(5-chloro-2-(4,4-difluoropiperidin-1-yl)-4-(trifluoromethyl)benzamido)-2-fluorophenyl)(methyl)(oxo)-λ⁶-sulfaneylidene)carbamate: To a solution of 5-chloro-2-(4,4-difluoropiperidin-1-yl)-4-(trifluoromethyl)benzoic acid (100 mg, 0.29 mmol), tert-butyl (R)-((5-amino-2-fluorophenyl)(methyl)(oxo)-16-sulfaneylidene)carbamate (126 mg, 0.44 mmol), HATU (166 mg, 0.44 mmol) and DIEA (113 mg, 0.87 mmol) in DMF (5 mL) stirred 25° C. for 16 h. After the reaction was completed, the mixture was diluted with water and extracted with EtOAc (50 mL×3). The combined organic layers were washed with brine, dried over Na₂SO₄, and concentrated under vacuum. The residue was purified by flash column chromatography on silica gel (PE/EtOAc=1/1) to give tert-butyl (R)-((5-(5-chloro-2-(4,4-difluoropiperidin-1-yl)-4-(trifluoromethyl)benzamido)-2-fluorophenyl)(methyl)(oxo)-λ⁶-sulfaneylidene)carbamate (100 mg, 50.9%) as yellow solid. LCMS (ESI) calcd. for C₂₅H₂₆ClF₆N₃NaO₄S [M+Na]⁺ m/z 636.11, found 636.0.

Step 3: (R)-5-chloro-2-(4,4-difluoropiperidin-1-yl)-N-(4-fluoro-3-(S-methylsulfonimidoyl)phenyl)-4-(trifluoromethyl)benzamide: A solution of tert-butyl (R)-((5-(5-chloro-2-(4,4-difluoropiperidin-1-yl)-4-(trifluoromethyl)benzamido)-2-fluorophenyl)(methyl)(oxo)-λ⁶-sulfaneylidene)carbamate (100 mg, 0.16 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (15 mL×3). The combined organic layers were washed with brine, dried over sodium sulfate, concentrated under vacuum. The residue was purified by prep-HPLC (Gemini 5 um C₁₈ column, 150*21.2 mm, eluting with 60% to 95% MeCN/H₂O containing 0.05% NH₃) to provide (R)-5-chloro-2-(4,4-difluoropiperidin-1-yl)-N-(4-fluoro-3-(S-methylsulfonimidoyl)phenyl)-4-(trifluoromethyl)benzamide (59.5 mg, 31.5%) as a white solid. ¹H NMR (400 MHz, DMSO-d₆) δ 10.86 (s, 1H), 8.41-8.29 (m, 1H), 7.98-7.88 (m, 1H), 7.80 (s, 1H), 7.56-7.41 (m, 2H), 4.70 (s, 1H), 3.26-3.16 (m, 7H), 2.10-1.93 (m, 4H). LCMS (ESI) calcd. for C₂₀H₁₉ClF₆N₃O₂S [M+H]⁺ m/z 514.08, found 514.0.

### Example 255

### (S)-5-chloro-2-(4,4-difluoropiperidin-1-yl)-N-(3-(S-methylsulfonimidoyl)phenyl)-4-(trifluoromethyl)benzamide

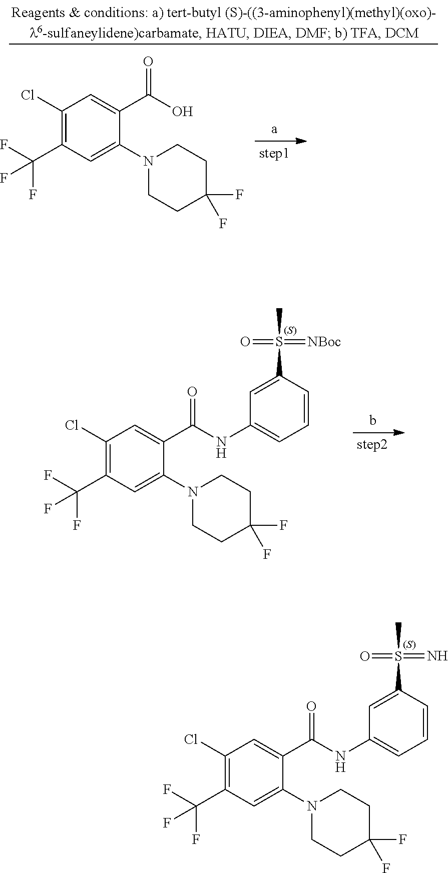

Step 1: tert-butyl (S)-((3-(5-chloro-2-(4,4-difluoropiperidin-1-yl)-4-(trifluoromethyl)benzamido)phenyl)(methyl)(oxo)-λ⁶-sulfaneylidene)carbamate: A solution of 5-chloro-2-(4,4-difluoropiperidin-1-yl)-4-(trifluoromethyl)benzoic acid (180 mg, 0.52 mmol), tert-butyl (S)-((3-aminophenyl)(methyl)(oxo)-λ⁶-sulfaneylidene)carbamate (213 mg, 0.78 mmol), HATU (299 mg, 0.78 mmol) and DIEA (203 mg, 1.57 mmol) in DMF (5 mL) was stirred at 25° C. for 16 hours. After the reaction was completed, the resulting solution was diluted with water (20 mL) and extracted with EtOAc (20 mL×3). The combined organic layers were washed with brine, dried over sodium sulfate, concentrated under vacuum. The residue was purified by flash column chromatography on silica gel (PE/EtOAc=1/1) to provide tert-butyl (S)-((3-(5-chloro-2-(4,4-difluoropiperidin-1-yl)-4-(trifluoromethyl)benzamido)phenyl)(methyl)(oxo)-λ⁶-sulfaneylidene)carbamate (120 mg, 38.3%) as yellow solid. LCMS (ESI) calcd. for C₂₅H₂₈ClF₅N₃O₄S [M+H]⁺ m/z 596.14, found 596.1.

Step 2: (S)-5-chloro-2-(4,4-difluoropiperidin-1-yl)-N-(3-(S-methylsulfonimidoyl)phenyl)-4-(trifluoromethyl)benzamide: A solution of tert-butyl (S)-((3-(5-chloro-2-(4,4-difluoropiperidin-1-yl)-4-(trifluoromethyl)benzamido)phenyl)(methyl)(oxo)-λ⁶-sulfaneylidene)carbamate (120 mg, 0.20 mmol) in DCM (5 mL) was added TFA (0.5 mL) at room temperature. The reaction mixture was stirred at room temperature for 1 hour. After the reaction was completed, the mixture was concentrated. The residue was adjusted to pH=8-9 with saturated aqueous NaHCO₃. Then the aqueous solution was extracted with DCM (15 mL×3). The combined organic layers were washed with brine, dried over sodium sulfate, concentrated under vacuum. The residue was purified by prep-HPLC (Gemini 5 um C₁₈ column, 150*21.2 mm, eluting with 45% to 90% MeCN/H₂O containing 0.05% NH₃) to provide (S)-5-chloro-2-(4,4-difluoropiperidin-1-yl)-N-(3-(S-methylsulfonimidoyl)phenyl)-4-(trifluoromethyl)benzamide (43 mg, 43.1%) as a white solid. ¹H NMR (400 MHz, DMSO-d₆) δ 10.86 (s, 1H), 8.41 (t, J=1.7 Hz, 1H), 7.90 (d, J=8.0 Hz, 1H), 7.81 (s, 1H), 7.69 (d, J=7.9 Hz, 1H), 7.62 (t, J=7.9 Hz, 1H), 7.53 (s, 1H), 4.23 (s, 1H), 3.26-3.14 (m, 4H), 3.06 (s, 3H), 2.10-1.95 (m, 4H). LCMS (ESI) calcd. for C₂₀H₂₀ClF₅N₃O₂S [M+H]⁺ m/z 496.09, found 496.1.

### Example 256

### (trans)-3-(2-(4,4-difluoroazepan-1-yl)-4-methyl-5-(trifluoromethyl)nicotinamido)cyclobutane-1-carboxylic acid

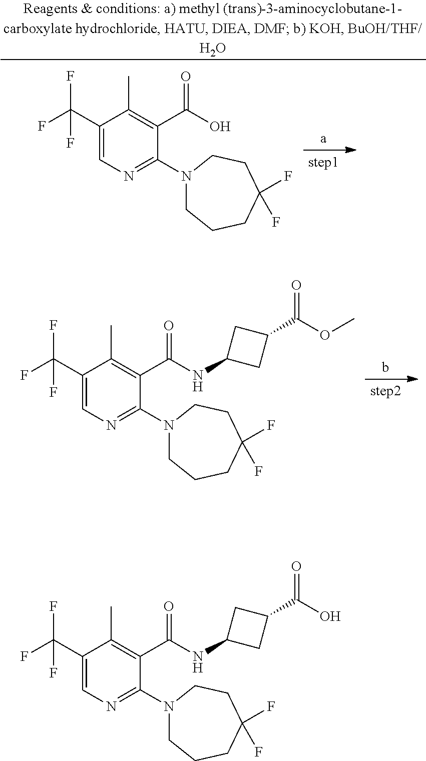 [SMILES: Cc1c(C(F)(F)F)cnc(N2CCCC(F)(F)CC2)c1C(=O)O ; COC(=O)[C@H]1C[C@H](NC(=O)c2c(N3CCCC(F)(F)CC3)ncc(C(F)(F)F)c2C)C1 ; Cc1c(C(F)(F)F)cnc(N2CCCC(F)(F)CC2)c1C(=O)N[C@H]1C[C@H](C(=O)O)C1]

Step 1: methyl (trans)-3-(2-(4,4-difluoroazepan-1-yl)-4-methyl-5-(trifluoromethyl)nicotinamido)cyclobutane-1-carboxylate: A mixture of 2-(4,4-difluoroazepan-1-yl)-4-methyl-5-(trifluoromethyl)nicotinic acid (200 mg, 0.59 mmol) and methyl (trans)-3-aminocyclobutane-1-carboxylate hydrochloride (117 mg, 0.71 mmol) in DMF (5 mL) was added HATU (293 mg, 0.77 mmol) and DIEA (244 mg, 1.89 mmol). The mixture was stirred at room temperature for 4 hours. The reaction was monitored by LCMS. After the reaction was completed, the resulting solution was diluted with water (15 mL) and extracted with DCM (15 mL×3). The combined organic layers were washed with brine, dried over Na₂SO₄, concentrated under vacuum. The residue was purified by flash column chromatography on silica gel (PE/EtOAc=2/1 to 1/1) to give methyl (trans)-3-(2-(4,4-difluoroazepan-1-yl)-4-methyl-5-(trifluoromethyl)nicotinamido)cyclobutane-1-carboxylate (180 mg, 0.68%) as white oil. LCMS (ESI) calcd. for C₂₀H₂₅F₅N₃O₃ [M+H]⁺ m/z 450.18, found 450.10.

Step 2: (trans)-3-(2-(4,4-difluoroazepan-1-yl)-4-methyl-5-(trifluoromethyl)nicotinamido)cyclobutane-1-carboxylic acid: A solution of methyl (trans)-3-(2-(4,4-difluoroazepan-1-yl)-4-methyl-5-(trifluoromethyl)nicotinamido)cyclobutane-1-carboxylate (180 mg, 0.40 mmol) in t-BuOH/THF/H₂O (1/1/1, 10 mL) was added KOH (224 mg, 4.0 mmol) at room temperature. The mixture was stirred for 2 hours at room temperature. The reaction was monitored by LCMS. After the reaction was completed, the mixture was cooled to room temperature, the reaction was extracted with DCM (20 mL), then the aqueous phase was adjusted to pH=3 with 1 N HCl and extracted with DCM (20 mL×3). The organic phase was dried with Na₂SO₄ and concentrated under vacuum to provide crude product (150 mg, 86.2% yield) as white solid. 1/3 of the above product (50 mg, 0.11 mmol) was further purified by prep-HPLC (Gemini 5 um C₁₈ column, 150*21.2 mm, eluting with 45% to 85% MeCN/H₂O containing 0.1% FA) to provide (trans)-3-(2-(4,4-difluoroazepan-1-yl)-4-methyl-5-(trifluoromethyl)nicotinamido)cyclobutane-1-carboxylic acid (7 mg) as white solid. ¹H NMR (400 MHz, DMSO-d₆) δ 12.27 (s, 1H), 8.94 (d, J=7.3 Hz, 1H), 8.36 (s, 1H), 4.53-4.41 (m, 1H), 3.71-3.57 (m, 4H), 3.00-2.89 (m, 1H), 2.46-2.40 (m, 2H), 2.32-2.17 (m, 7H), 2.07-1.94 (m, 2H), 1.89-1.80 (m, 2H). LCMS (ESI) calcd. for C₁₉H₂₃F₅N₃O₃ [M+H]⁺ m/z 436.17, found 436.2.

### Example 257

### N-((trans)-3-carbamoylcyclobutyl)-2-(4,4-difluoroazepan-1-yl)-4-methyl-5-(trifluoromethyl)nicotinamide

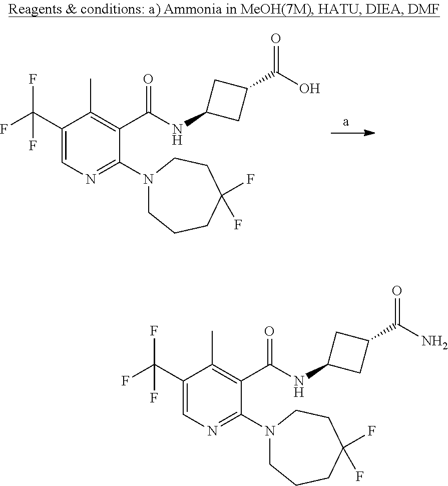 [SMILES: Cc1c(C(F)(F)F)cnc(N2CCCC(F)(F)CC2)c1C(=O)N[C@H]1C[C@H](C(=O)O)C1 ; Cc1c(C(F)(F)F)cnc(N2CCCC(F)(F)CC2)c1C(=O)N[C@H]1C[C@H](C(N)=O)C1]

A mixture of (trans)-3-(2-(4,4-difluoroazepan-1-yl)-4-methyl-5-(trifluoromethyl)nicotinamido)cyclobutane-1-carboxylic acid (100 mg, 0.23 mmol) and HATU (131 mg, 0.34 mmol) in DMF (5 mL) was added NH₃-MeOH (7M, 2 mL) and DIEA (148.4 mg, 1.15 mmol). The mixture was stirred at room temperature for 2 hours. The reaction was monitored by LCMS. After the reaction was completed, the resulting solution was diluted with water (15 mL) and extracted with DCM (15 mL×3). The combined organic layers were washed with brine, dried over Na₂SO₄, concentrated under vacuum. The residue was purified by prep-HPLC (Gemini 5 um C₁₈ column, 150*21.2 mm, eluting with 45% to 70% MeCN/H₂O containing 0.05% NH₃) to provide N-((trans)-3-carbamoylcyclobutyl)-2-(4,4-difluoroazepan-1-yl)-4-methyl-5-(trifluoromethyl)nicotinamide (27 mg, 27.1%) as white solid. ¹H NMR (400 MHz, DMSO-d₆) δ 8.87 (d, J=7.2 Hz, 1H), 8.36 (s, 1H), 7.24 (s, 1H), 6.80 (s, 1H), 4.56-4.37 (m, 1H), 3.72-3.57 (m, 4H), 2.93-2.82 (m, 1H), 2.41-2.34 (m, 2H), 2.32-2.20 (m, 5H), 2.18-2.10 (m, 2H), 2.07-1.97 (m, 2H), 1.89-1.78 (m, 2H). LCMS (ESI) calcd. for C₁₉H₂₄F₅N₄O₂ [M+H]⁺ m/z 435.18, found 435.1

### Example 258

### (R)-4-methyl-N-(2-(S-methylsulfonimidoyl)pyridin-4-yl)-2-(4-(2,2,2-trifluoroethyl)-1,4-diazepan-1-yl)-5-(trifluoromethyl)nicotinamide

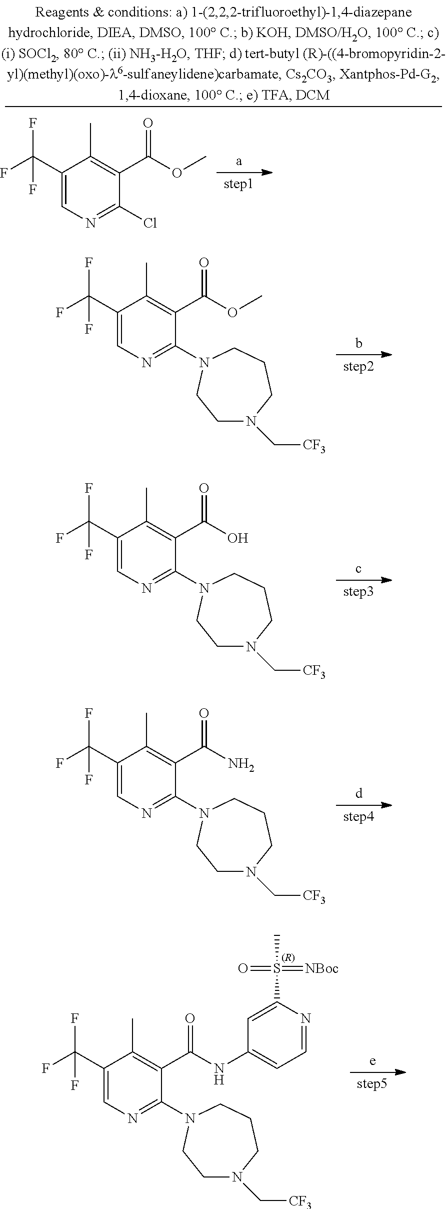

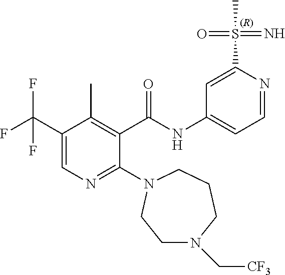

Step 1: methyl 4-methyl-2-(4-(2,2,2-trifluoroethyl)-1,4-diazepan-1-yl)-5-(trifluoromethyl)nicotinate: A solution of methyl 2-chloro-4-methyl-5-(trifluoromethyl)nicotinate (1 g, 3.91 mmol), 1-(2,2,2-trifluoroethyl)-1,4-diazepane hydrochloride (1.28 g, 5.85 mmol) and DIEA (2.52 g, 19.52 mmol) in DMSO (20 mL) was heated to 100° C. and stirred for 16 hours. After the reaction was completed, the mixture was cooled to room temperature. The resulting solution was diluted with water (100 mL) and extracted with EtOAc (100 mL×3). The combined organic layers were washed with brine, dried over sodium sulfate, concentrated under vacuum. The residue was purified by flash column chromatography on silica gel (PE/EtOAc=1/1) to provide methyl 4-methyl-2-(4-(2,2,2-trifluoroethyl)-1,4-diazepan-11-yl)-5-(trifluoromethyl)nicotinate (900 mg, 58.97%) as yellow oil. LCMS (ESI) calcd. for C₁₆H₂₀F₆N₃O 2 [M+H]⁺ m/z 400.15, found 400.0.

Step 2: 4-methyl-2-(4-(2,2,2-trifluoroethyl)-1,4-diazepan-1-yl)-5-(trifluoromethyl)nicotinic acid: A solution of methyl 4-methyl-2-(4-(2,2,2-trifluoroethyl)-1,4-diazepan-1-yl)-5-(trifluoromethyl)nicotinate (500 mg, 1.25 mmol) in DMSO/H₂O (1/1, 10 mL) was added KOH (3.51 g, 62.61 mmol). Then the solution was heated to 100° C. and stirred for 16 hours. After the reaction was completed, the mixture was cooled to room temperature. The resulting solution was adjusted to pH=3-4 with saturated HCl (1 M) and extracted with EtOAc (100 mL×3). The combined organic layers were washed with brine, dried over sodium sulfate, concentrated under vacuum to give 4-methyl-2-(4-(2,2,2-trifluoroethyl)-1,4-diazepan-1-yl)-5-(trifluoromethyl)nicotinic acid (400 mg, 89.55%) as yellow solid which was used without further purification. LCMS (ESI) calcd. for C₁₅H₁₈F₆N₃O₂ [M+H]⁺ m/z 386.13, found 385.7.

Step 3: 4-methyl-2-(4-(2,2,2-trifluoroethyl)-1,4-diazepan-1-yl)-5-(trifluoromethyl)nicotinamide: A solution of 4-methyl-2-(4-(2,2,2-trifluoroethyl)-1,4-diazepan-1-yl)-5-(trifluoromethyl)nicotinic acid (380 mg, 0.99 mmol) in SOCl₂ (10 mL) was heated to 80° C. and stirred for 2 hours. The solution was concentrated under vacuum to provide the chloride intermediate. Then the chloride intermediate dissolved in THE (10 mL) was added to NH₃·H₂O (3 mL) at 0° C. The resulting mixture was stirred at 25° C. for 1 hour. Then the mixture was quenched with water (20 mL) and extracted with DCM (20 mL×3). The combined organic layers were washed with brine, dried over sodium sulfate, concentrated under vacuum. The residue was purified by flash column chromatography on silica gel (PE/EtOAc=3/2) to provide 4-methyl-2-(4-(2,2,2-trifluoroethyl)-1,4-diazepan-1-yl)-5-(trifluoromethyl)nicotinamide (200 mg, 52.77%) as yellow oil. LCMS (ESI) calcd. for C₁₅H₁₉F₆N₄O [M+H]⁺ m/z 385.15, found 385.1.

Step 4: tert-butyl (R)-(methyl(4-(4-methyl-2-(4-(2,2,2-trifluoroethyl)-1,4-diazepan-1-yl)-5-(trifluoromethyl)nicotinamido)pyridin-2-yl)(oxo)-λ⁶-sulfaneylidene)carbamate: A mixture of 4-methyl-2-(4-(2,2,2-trifluoroethyl)-1,4-diazepan-1-yl)-5-(trifluoromethyl)nicotinamide (200 mg, 0.52 mmol), tert-butyl (R)-((4-bromopyridin-2-yl)(methyl)(oxo)-λ⁶-sulfaneylidene)carbamate (262 mg, 0.78 mmol), Cs₂CO₃ (452 mg, 1.39 mmol) and Xantphos-Pd-G2 (98 mg, 0.11 mmol) in 1,4-dioxane (5 mL) was stirred at 100° C. for 2 hours under nitrogen. After the reaction was completed, the mixture was filtered through celite. The filtrate was concentrated and residue was purified by flash column chromatography on silica gel (PE/EtOAc=1/1) to provide tert-butyl (R)-(methyl(4-(4-methyl-2-(4-(2,2,2-trifluoroethyl)-1,4-diazepan-1-yl)-5-(trifluoromethyl)nicotinamido)pyridin-2-yl)(oxo)-λ⁶-sulfaneylidene)carbamate (220 mg, 66.08%) as a yellow solid. LCMS (ESI) calcd. for C₂₆H₃₃F₆N₆O₄S [M+H]⁺ m/z 639.22, found 639.2.

Step 5: (R)-4-methyl-N-(2-(S-methylsulfonimidoyl)pyridin-4-yl)-2-(4-(2,2,2-trifluoroethyl)-1,4-diazepan-1-yl)-5-(trifluoromethyl)nicotinamide: A solution of tert-butyl (R)-(methyl(4-(4-methyl-2-(4-(2,2,2-trifluoroethyl)-1,4-diazepan-1-yl)-5-(trifluoromethyl)nicotinamido)pyridin-2-yl)(oxo)-λ⁶-sulfaneylidene)carbamate (210 mg, 0.33 mmol) in DCM (5 mL) was added TFA (0.5 mL) at room temperature. The reaction mixture was stirred at room temperature for 1 hour. After the reaction was completed, the mixture was concentrated. The residue was adjusted to pH=8-9 with saturated aqueous NaHCO₃. Then the aqueous solution was extracted with DCM (15 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.05% NH₃) to provide (R)-4-methyl-N-(2-(S-methylsulfonimidoyl)pyridin-4-yl)-2-(4-(2,2,2-trifluoroethyl)-1,4-diazepan-1-yl)-5-(trifluoromethyl)nicotinamide (65.0 mg, 36.77%) as a white solid. ¹H NMR (400 MHz, DMSO-d₆) δ 11.36 (s, 1H), 8.63 (d, J=5.4 Hz, 1H), 8.45 (s, 1H), 8.40 (d, J=1.8 Hz, 1H), 7.81 (dd, J=5.5, 2.0 Hz, 1H), 4.39 (s, 1H), 3.69-3.59 (m, 4H), 3.30-3.21 (m, 2H), 3.15 (s, 3H), 3.00-2.91 (m, 2H), 2.79-2.72 (m, 2H), 2.30 (s, 3H), 1.85-1.76 (m, 2H). LCMS (ESI) calcd. for C₂₁H₂₅F₆N₆O₂S [M+H]⁺ m/z 539.17, found 539.2.

### Example 259

### N-((cis)-3-carbamoylcyclobutyl)-2-(4,4-difluoroazepan-1-yl)-4-methyl-5-(trifluoromethyl)nicotinamide

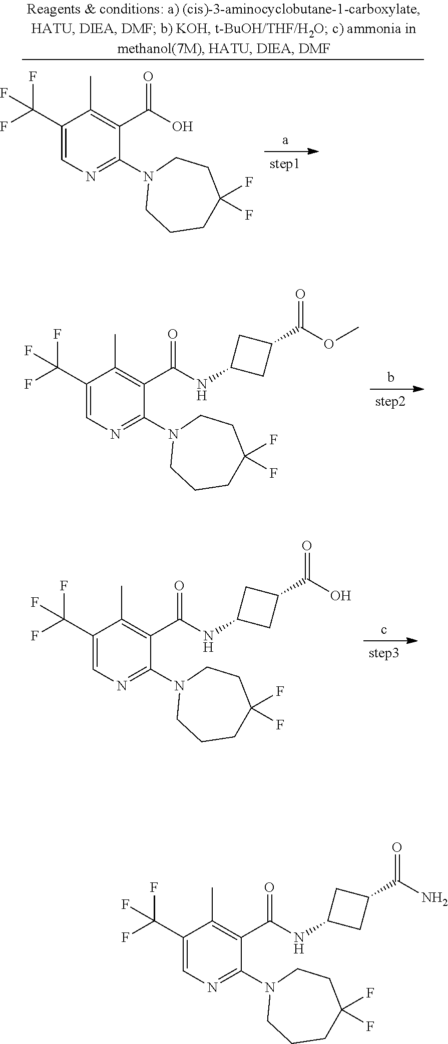 [SMILES: Cc1c(C(F)(F)F)cnc(N2CCCC(F)(F)CC2)c1C(=O)O ; COC(=O)[C@H]1C[C@@H](NC(=O)c2c(N3CCCC(F)(F)CC3)ncc(C(F)(F)F)c2C)C1 ; Cc1c(C(F)(F)F)cnc(N2CCCC(F)(F)CC2)c1C(=O)N[C@H]1C[C@@H](C(=O)O)C1 ; Cc1c(C(F)(F)F)cnc(N2CCCC(F)(F)CC2)c1C(=O)N[C@H]1C[C@@H](C(N)=O)C1]

Step 1: methyl (cis)-3-(2-(4,4-difluoroazepan-1-yl)-4-methyl-5-(trifluoromethyl)nicotinamido)cyclobutane-1-carboxylate: A mixture of 2-(4,4-difluoroazepan-1-yl)-4-methyl-5-(trifluoromethyl)nicotinic acid (200 mg, 0.59 mmol) and methyl (cis)-3-aminocyclobutane-1-carboxylate (153 mg, 1.18 mmol) in DMF (5 mL) was added HATU (337 mg, 0.89 mmol) and DIEA (229 mg, 1.77 mmol). The mixture was heated to 80° C. and stirred for 24 hours. The reaction was monitored by LCMS. After the reaction was completed, the resulting solution was diluted with water (15 mL) and extracted with DCM (15 mL×3). The combined organic layers were washed with brine, dried over Na₂SO₄, concentrated under vacuum. The residue was purified by flash column chromatography on silica gel (PE/EtOAc=2/1 to 1/1) to give methyl (cis)-3-(2-(4,4-difluoroazepan-1-yl)-4-methyl-5-(trifluoromethyl)nicotinamido)cyclobutane-1-carboxylate (150 mg, 56.9%) as a yellow solid. LCMS (ESI) calcd. for C₂₀H₂₅F₅N₃O₃ [M+H]⁺ m/z 450.18, found 450.1.

Step 2: (cis)-3-(2-(4,4-difluoroazepan-1-yl)-4-methyl-5-(trifluoromethyl)nicotinamido)cyclobutane-1-carboxylic acid: To a solution of methyl (cis)-3-(2-(4,4-difluoroazepan-1-yl)-4-methyl-5-(trifluoromethyl)nicotinamido)cyclobutane-1-carboxylate (150 mg, 0.33 mmol) in t-BuOH/THF/H₂O (1:1:1, 10 mL) was added KOH (187.3 mg, 3.34 mmol) at room temperature. The mixture was stirred for 2 hours at room temperature. The reaction was monitored by LCMS. After the reaction was completed, the mixture was cooled to room temperature, the reaction was extracted with DCM (20 mL), then the aqueous phase was adjusted to pH=3 with 1 N HCl and extracted with DCM (20 mL×3). The combined organic layers were dried with Na₂SO₄ and concentrated under vacuum to give (cis)-3-(2-(4,4-difluoroazepan-1-yl)-4-methyl-5-(trifluoromethyl)nicotinamido)cyclobutane-1-carboxylic acid (140 mg, 96.3%) as a white solid. LCMS (ESI) calcd. for C₁₉H₂₃F₅N₃O₃ [M+H]⁺ m/z 436.17, found 436.10.

Step 3: N-((cis)-3-carbamoylcyclobutyl)-2-(4,4-difluoroazepan-1-yl)-4-methyl-5-(trifluoromethyl)nicotinamide: To a solution of (cis)-3-(2-(4,4-difluoroazepan-1-yl)-4-methyl-5-(trifluoromethyl)nicotinamido)cyclobutane-1-carboxylic acid (140 mg, 0.32 mmol) and HATU (183 mg, 0.48 mmol) in DMF (5 mL) was added NH₃-MeOH (7M, 5 mL) and DIEA (208 mg, 1.61 mmol). The mixture was stirred at room temperature for 2 hours. The reaction was monitored by LCMS. After the reaction was completed, the resulting solution was diluted with water (15 mL) and extracted with DCM (15 mL×3). The combined organic layers were washed with brine, dried over Na₂SO₄, concentrated under vacuum. The residue was purified by prep-HPLC (Gemini 5 um C₁₈ column, 150*21.2 mm, eluting with 40% to 60% MeCN/H₂O containing 0.05% NH₃) to provide N-((cis)-3-carbamoylcyclobutyl)-2-(4,4-difluoroazepan-1-yl)-4-methyl-5-(trifluoromethyl)nicotinamide (28.6 mg, 20.5%) as white solid. ¹H NMR (400 MHz, DMSO-d₆) δ 8.87 (d, J=7.3 Hz, 1H), 8.35 (s, 1H), 7.22 (s, 1H), 6.75 (s, 1H), 4.33-4.14 (m, 1H), 3.74-3.55 (m, 4H), 2.73-2.62 (m, 1H), 2.40-2.19 (m, 7H), 2.10-1.97 (m, 4H), 1.89-1.76 (m, 2H). LCMS (ESI) calcd. for C₁₉H₂₄F₅N₄O₂ [M+H]⁺ m/z 435.18, found 435.1.

### Potency of the Compounds:

The potency of the examples listed above are assayed against human Nav1.8 using either SyncroPatch384PE assay or Sophion QPatch II assay described below.

### SyncroPatch384PE Assay

Compounds were tested on recombinant human Nav1.8/b3 stably transfected CHO cells using the SyncroPatch384PE system (Nanion Technologies), an automated patch clamp device. Cells were cultured at 37° C./5% CO₂ in Ham's F-12 supplemented with 10% fetal bovine serum, 100 U/mL penicillin G sodium, 100 mg/mL streptomycin sulfate and selection antibiotics (0.01 mg/mL Blasticidin, 0.4 mg/mL Zeocin and 0.25 mg/mL Hygromycin). On the day of the recordings, cells in culture dishes were washed twice with Hank's Balanced Salt Solution (HBSS) and treated with Accutase for approximately 20 minutes. Immediately before use in the SynchroPatch384PE using an 8-hole NPC-384 chip, the cells were washed in HBSS to remove the Accutase and re-suspended in extracellular solution. All experiments were performed at ambient temperature. Intracellular solution contained (mM): CsCl, 50; CsF, 90; MgCl₂, 5; EGTA, 1; HEPES, 10; pH adjusted to 7.2 with CsOH. Extracellular solution contained (mM): NaCl, 137; KCl, 4.0; CaCl₂), 3.8; MgCl₂, 1; HEPES, 10; Glucose, 10; pH adjusted to 7.4 with NaOH. 100 nM tetrodotoxin (TTX) was added to the extracellular solution to block endogenous TTX-sensitive sodium currents.

Compounds were tested in quadruplicate in 0.3% DMSO and 0.03% pluronic Acid. Compounds were diluted 1:3.33 in extracellular solution to create an 8-point concentration response curve. Each plate contained a historical positive control and up to ten compounds. 300 μM tetracaine and 0.3% DMSO+0.03% pluronic acid were used as high and low controls respectively.

Whole cell patch clamp recordings were conducted according to Nanion's standard procedure for SyncroPatch384PE®. Cells were held at a holding potential of −120 mV. A depolarization step to 10 mV for 30 ms was applied (P1 measurement), followed by a hyperpolarization step to −100 mV for 100 ms. An inactivation step at −35 mV for 10 sec was applied before stepping to −100 mV for 20 ms, followed by a step to 10 mV for 30 ms (P2 measurement) and then back to −100 mV for 30 ms. Sweep interval was 15 sec. Following establishment of the whole-cell configuration in extracellular solution, cells were washed in extracellular solution containing 0.3% DMSO and 0.03% pluronic acid to stabilize the baseline current. Compounds were then applied by the SynchroPatch384 PE system into each well and the current was recorded for five minutes in extracellular solution, followed by application of tetracaine to achieve full block at the end of experiment. The potency of the compounds was assessed on two read-outs, resting state block (P1 measurement) or inactivated state block (P2 measurement) to obtain IC₅₀ values. Values were normalized to high (tetracaine) and low (DMSO+pluronic acid) controls.

Examples 1 to 219 and Examples 225 to 236 are assayed using SyncroPatch384PE assay described above.

### Sophion Qpatch II Assay

Compounds were tested on recombinant human Nav1.8/01 stably transfected HEK293 (Eurofins, CYL3025, St. Charles, MO) cells using the Qpatch II system (Sophion Bioscience A/S, Ballerup—Denmark), an automated patch clamp device. Cells were cultured at 37° C./5% CO₂ in DMEM/F-12 supplemented with 10% fetal bovine serum, lx Non-Essential Amino Acids, and selection antibiotics (0.625 μg/mL Puromycin, 400 μg/mL Geneticin, and 100 μg/mL Hygromycin). One or two days prior to recording, cells were moved to a lower temperature 30° C./5% CO₂ incubator to increase surface expression of the channels. On the day of the recordings, cells in culture dishes were washed twice with Ca²⁺—Mg²⁺ free DPBS and treated with Detachin (Genlantis, San Diego) for 3 minutes and then resuspended in Serum Free Medium (EX-CELL® ACF CHO Medium for Cell Culture (Sigma-Aldrich) supplemented with 0.04 mg/mL soybean trypsin Inhibitor and 25 mM HEPES (all components supplied by Sigma-Aldrich, St. Louis, MO) and allowed to recover for a minimum of 20 minutes in the onboard Qstirrer. All experiments were performed at ambient temperature. Intracellular solution contained (mM): CsCl, 50; CsF, 90; MgCl₂, 2; EGTA, 5; HEPES, 10; pH adjusted to 7.2 with CsOH. Extracellular solution contained (mM): NaCl, 137; KCl, 4; CaCl₂), 3.8; MgCl₂, 1; HEPES, 10; Glucose, 10; pH adjusted to 7.4 with NaOH. 500 nM tetrodotoxin (TTX) was added to the extracellular solution to block endogenous TTX-sensitive sodium currents.

Compounds were tested in an interleaved manner across two cohorts of wells to develop an aggregate 8-point concentration response curve. Final concentrations include 0.5% DMSO and 0.03% pluronic Acid. The concentration range spanned a range from 0.01-300 nM. Compounds were diluted 1:10 with extracellular solution within their respective cohort. (R)-2-(4-fluoro-2-methylphenoxy)-N-(3-(S-methylsulfonimidoyl)phenyl)-5-(trifluoromethyl)nicotinamide served as a positive control while DMSO (0.5% by volume) served as a vehicle control to ascertain assay performance.

Whole cell patch clamp recordings were conducted according to Sophion standard procedure for Qpatch II®. Cells were held at a holding potential of −120 mV. A depolarizing step to 10 mV for 30 ms was applied (TP1 measurement), followed by a hyperpolarization step to −100 mV for 20 ms. Due to instability observed at long depolarized potentials, there were no second TP2 measurements attempted. Inter-sweep interval was 15 sec. Following establishment of the whole-cell configuration in extracellular solution, cells were washed twice in extracellular solution containing 0.5% DMSO and 0.03% pluronic acid to stabilize the baseline current. Compounds were then applied by the Qpatch II system into each well in an ascending manner within cohort and the current was recorded for five minutes for each concentration. Percent of current remaining was determined by taking the average of the last 5 sweeps within each concentration and normalizing to the latest pre-compound saline period. The potency of the compounds was assessed on resting state block (TP1 measurement) only to obtain IC₅₀ values. Wells whose initial sodium current magnitude were below 2 nA or whole cell resistance fell below 100 MQ were excluded from analysis.

Examples 220 to 224, Examples 237 to 257 and Example 259 are assayed using Sophion Qpatch II assay described above.

Table 1 shows the potency of compounds against human Nav1.8, where “A” represents an IC₅₀ less than or equal to 5 nM, “B” represents an IC₅₀ greater than 5 nM to less than or equal to 50 nM, “C” represents an IC₅₀ greater than 50 nM to less than or equal to 100 nM, “D” represents an IC₅₀ greater than 100 nM to less than or equal to 200 nM, “E” represents an IC₅₀ greater than 200 nM.

TABLE 1

| Example | P1 IC50 | P2 IC50 | Example | P1 IC50 | P2 IC50 |
|---|---|---|---|---|---|
| 1 | E | E | 2 | E | E |
| 3 | A | A | 4 | A | A |
| 5 | A | A | 6 | B | B |
| 7 | D | D | 8 | E | E |
| 9 | E | E | 10 | C | C |
| 11 | D | D | 12 | A | A |
| 13 | A | A | 14 | E | E |
| 15 | A | A | 16 | A | A |
| 17 | A | A | 18 | E | D |
| 19 | E | D | 20 | C | C |
| 21 | E | E | 22 | E | E |
| 25 | A | A | 26 | A | A |
| 27 | A | A | 28 | B | B |
| 29 | B | B | 30 | A | A |
| 31 | E | E | 32 | E | E |
| 33 | A | A | 34 | A | A |
| 35 | A | A | 36 | E | E |
| 37 | E | E | 38 | E | E |
| 39 | E | E | 40 | E | E |
| 41 | A | A | 42 | A | A |
| 43 | A | A | 44 | B | B |
| 45 | C | C | 46 | B | B |
| 47 | B | B | 48 | A | A |
| 49 | A | A | 50 | A | A |
| 51 | B | B | 52 | B | B |
| 53 | B | B | 54 | B | B |
| 55 | A | A | 56 | A | A |
| 57 | A | A | 58 | A | A |
| 59 | A | A | 60 | A | A |
| 61 | A | A | 62 | A | A |
| 63 | A | A | 64 | A | A |
| 65 | A | A | 66 | A | A |
| 67 | A | A | 68 | A | A |
| 69 | B | B | 70 | A | A |
| 71 | E | E | 72 | C | C |
| 73 | C | B | 74 | B | B |
| 75 | B | B | 76 | A | A |
| 77 | A | A | 78 | E | E |
| 79 | E | E | 80 | B | B |
| 81 | A | A | 82 | A | A |
| 83 | C | C | 84 | C | B |
| 85 | C | C | 86 | A | A |
| 87 | A | A | 88 | A | A |
| 89 | D | D | 90 | D | D |
| 91 | B | B | 92 | A | A |
| 93 | A | A | 94 | E | E |
| 95 | A | A | 96 | A | A |
| 97 | B | B | 98 | B | B |
| 99 | D | D | 100 | E | E |
| 101 | A | A | 102 | B | B |
| 103 | A | A | 104 | B | B |
| 105 | C | C | 106 | A | A |
| 107 | A | A | 108 | A | A |
| 109 | A | A | 110 | A | A |
| 111 | A | A | 112 | C | C |
| 113 | A | A | 114 | A | A |
| 115 | A | A | 116 | E | E |
| 117 | D | C | 118 | C | C |
| 119 | B | B | 120 | D | C |
| 121 | A | A | 122 | A | A |
| 123 | E | E | 124 | D | D |
| 125 | B | B | 126 | A | A |
| 127 | A | A | 128 | E | E |
| 129 | E | E | 130 | A | A |
| 131 | A | A | 132 | E | E |
| 133 | A | A | 134 | E | E |
| 135 | B | B | 136 | A | A |
| 137 | E | E | 138 | E | D |
| 139 | E | D | 140 | E | E |
| 141 | A | A | 142 | A | A |
| 143 | C | C | 144 | E | E |
| 145 | A | A | 146 | E | E |
| 147 | B | B | 148 | B | B |
| 149 | B | B | 150 | D | D |
| 151 | D | D | 152 | E | E |
| 153 | E | E | 154 | C | C |
| 155 | E | E | 156 | E | E |
| 157 | E | E | 158 | C | C |
| 159 | B | B | 160 | B | B |
| 161 | E | E | 162 | A | A |
| 163 | A | A | 164 | A | A |
| 165 | B | B | 166 | A | A |
| 167 | B | B | 168 | B | B |
| 169 | A | A | 170 | B | B |
| 171 | A | A | 172 | A | A |
| 173 | E | D | 174 | B | B |
| 175 | B | B | 176 | A | A |
| 177 | A | A | 178 | B | B |
| 179 | A | A | 180 | A | A |
| 181 | A | A | 182 | A | A |
| 183 | A | A | 184 | A | A |
| 185 | A | A | 186 | A | A |
| 187 | A | A | 188 | A | A |
| 189 | A | A | 190 | A | A |
| 191 | A | A | 192 | A | A |
| 193 | A | A | 194 | A | A |
| 195 | A | A | 196 | A | A |
| 197 | C | C | 198 | A | A |
| 199 | A | A | 200 | D | D |
| 201 | B | B | 202 | A | A |
| 203 | B | B | 204 | B | B |
| 205 | A | A | 206 | A | A |
| 207 | B | B | 208 | B | B |
| 209 | A | A | 210 | B | B |
| 211 | A | A | 212 | B | B |
| 213 | D | D | 214 | D | D |
| 215 | B | B | 216 | C | C |
| 217 | C | C | 218 | B | B |
| 219 | A | A | 220 | B | |
| 221 | B | | 222 | B | |
| 223 | A | | 224 | B | |
| 225 | B | B | 226 | A | A |
| 227 | A | A | 228 | A | A |
| 229 | B | B | 230 | B | B |
| 231 | B | B | 232 | A | A |
| 233 | A | A | 234 | A | A |
| 235 | A | A | 236 | A | A |
| 237 | C | | 238 | E | |
| 239 | E | | 240 | B | |
| 241 | B | | 242 | B | |
| 243 | C | | 244 | C | |
| 245 | B | | 246 | C | |
| 247 | D | | 248 | C | |
| 249 | C | | 250 | A | |
| 251 | C | | 252 | C | |
| 254 | B | | 255 | B | |
| 256 | C | | 257 | C | |
| 259 | C | | | | |

### Plasma Concentration

It is well known that plasma protein binding modulates the effective drug concentrations at pharmacological target sites (J. Pham Sci (2013) 102, 2953-2994). Free drug theory holds that, absent active uptake mechanisms, free drug in plasma is equal to free drug concentration in tissues and only unbound drug in tissue is contributory to target receptor binding and pharmacologic activity (Expert Opin. Drug Discov. (2007) 2, 51-64). In general, drugs of a given potency against a biological target having reduced plasma protein binding can affect pharmacology with lower total plasma concentrations than comparable drugs with higher plasma protein binding. Hence, increasing the plasma free fraction can have a favorable impact on a drug's effective modulation of a biological target and the resulting pharmacologic activity per dose.

In the present invention, without being bound by any mechanism, unexpected increases in plasma drug free fraction (reduced drug-plasma protein binding) have been found for compounds of Formula I when R₂ is a sulfoximine. For example, in certain embodiments of the invention, wherein R₂ is

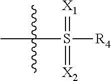

and wherein, X₁ is O and X₂ is NH. As demonstrated from the data below, the compounds comprising R₂ is sulfoximine has a better free plasma concentration as compared to the compounds of Formula (I), wherein R₂ is sulfonamido, i.e., (X₁ and X₂ are O and R₄ is NH₂).

Table 2 presents sulfoximine compounds of the invention alongside comparator sulfonamide compounds. Table 3 presents the human and rat plasma protein binding data (% unbound) for the compounds of Table 2.

TABLE 2
Comparative Sulfoximine and Sulfonamides

| Sulfoximine of Current Invention | Comparator Sulfonamide |
|---|---|
| 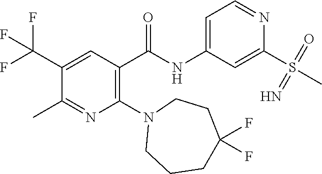 [SMILES: Cc1nc(N2CCCC(F)(F)CC2)c(C(=O)Nc2ccnc(S(C)(=N)=O)c2)cc1C(F)(F)F] | 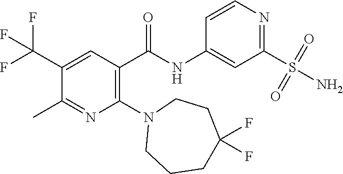 [SMILES: Cc1nc(N2CCCC(F)(F)CC2)c(C(=O)Nc2ccnc(S(N)(=O)=O)c2)cc1C(F)(F)F] |
| Ex 101 | Compound A |
| 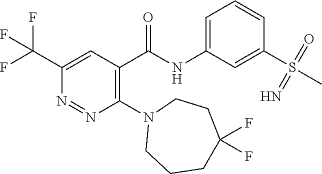 [SMILES: CS(=N)(=O)c1cccc(NC(=O)c2cc(C(F)(F)F)nnc2N2CCCC(F)(F)CC2)c1] | 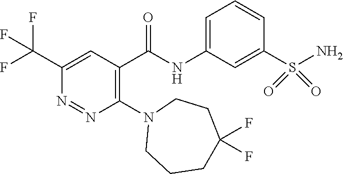 [SMILES: NS(=O)(=O)c1cccc(NC(=O)c2cc(C(F)(F)F)nnc2N2CCCC(F)(F)CC2)c1] |
| Compound E | Compound B |
| 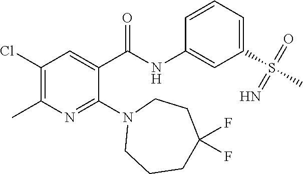 | 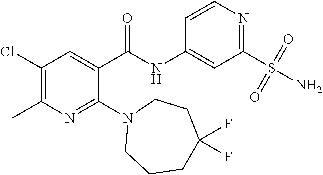 [SMILES: Cc1nc(N2CCCC(F)(F)CC2)c(C(=O)Nc2ccnc(S(N)(=O)=O)c2)cc1Cl] |
| Example 17 | Compound C |
| 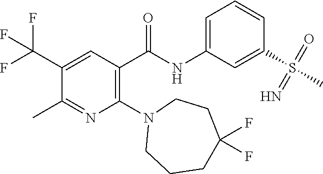 [SMILES: Cc1nc(N2CCCC(F)(F)CC2)c(C(=O)Nc2cccc([S@](C)(=N)=O)c2)cc1C(F)(F)F] | 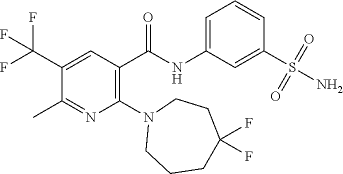 [SMILES: Cc1nc(N2CCCC(F)(F)CC2)c(C(=O)Nc2cccc(S(N)(=O)=O)c2)cc1C(F)(F)F] |
| Example 4 | Compound D |

Comparator compounds A, B, C have been previously reported as inhibitors of Na, 1.8 in WO2020/092667. Comparator D, not previously reported, is offered as an additional sulfonamido example. As indicated in Table 3, sulfoximines of the present invention, namely Example 101, Example 17 and Example 4 exhibit free fractions of 1.8, 1.7, and 0.9% in an assay measuring human plasma protein binding. Further, Example 101, Compound E, Example 4, and Example 17 exhibit plasma free fractions of 3.55, 37.8, 3.0, and 1.9% respectively in an assay measuring rat plasma protein binding. In contrast, the comparator sulfonamido Compounds A, B, C, and D have been found to exhibit plasma free fractions of 0.24, 17.4, 0.53, and 0.31% respectively in an assay measuring human plasma protein binding and 0.52, 15.8, 1.62, and 0.31% respectively in an assay measuring rat plasma protein binding.

TABLE 3
Plasma Free fractions of Compounds of
Formula I vs Comparator Compounds

| | Human Plasma, | Fold increase vs | Rat Plasma, | Fold increase vs |
|---|---|---|---|---|
| Compound | Unbound %¹ | Comparatror² | Unbound %³ | Comparator⁴ |
| Example 101 | 1.8 | 7.5× | 3.55 | 6.8× |
| Compound E⁵ | NT | NA | 37.8 | 2.4× |
| Example 17 | 1.7 | 3.2× | 3.0 | 1.9× |
| Example 4 | 0.9 | 2.9× | 1.9 | 6.1× |

Comparators

| | | | | |
|---|---|---|---|---|
| Compound A | 0.24 | NA | 0.52 | NA |
| (WO2020/092667, | | | | |
| Ex 184) | | | | |
| Compound B | 17.4 | NA | 15.8 | NA |
| (WO2020/092667, | | | | |
| Ex 179) | | | | |
| Compound C | 0.53 | NA | 1.62 | NA |
| (WO2020/092667, | | | | |
| Ex 172) | | | | |
| Compound D | 0.31 | NA | 0.31 | NA |

¹Human Plasma binding was measured by equilibrium dialysis as generally described by Jitendra Kumar et al., J Bioequiv Availab (2012), S14.
²Fold increase is reported as the ratios of Ex 101/Compound A; Ex 17/Compound C; Ex 4/Compound D
³Rat Plasma binding was measured by equilibrium dialysis as generally described by Jitendra Kumar et al., J Bioequiv Availab (2012), S14.
⁴Fold increase is reported as the ratios of Ex 101/Compound A; Compound E/Compound B; Ex 17/Compound C; Ex 4/Compound D
⁵Compound E is the racemate of Examples 18 and 19.
NT = Not tested
NA = Not applicable

Thus, on a comparable structural basis, sulfoximine compounds of the invention in Table 3 exhibit 2.9-7.5 fold higher plasma free fractions in human plasma and 1.9-6.8 fold higher plasma free fractions in rat plasma than sulfonamide comparator compounds. This unexpected increase in plasma free fraction improves the drug-like properties of sulfoximines of Formula I versus analogous sulfonamides.

### INCORPORATION BY REFERENCE

References and citations to other documents, such as patents, patent applications, patent publications, journals, books, papers, web contents, have been made throughout this disclosure.

All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

1. A compound of Formula (I): 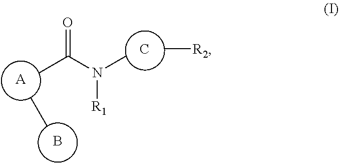
wherein:
A is an aryl or heteroaryl ring containing one or more heteroatoms independently selected from O, S, and N; wherein A is unsubstituted or substituted with one or more substituents selected from a group consisting of:
H, halo, C₁-C₆ alkyl, C₁-C₆ branched alkyl, C₁-C₆ alkenyl, C₁-C₆ alkynyl, C₁-C₆ haloalkyl, C₁-C₆ alkoxy, C₁-C₆ cycloalkoxy, C₁-C₆ haloalkoxy, nitro, cyano, —CH₂-(C₁-C₆)-cycloalkyl, —CF₂-cycloalkyl, —CH(CH₃)-cycloalkyl, —CH₂-aryl, —CF₂-aryl, —CH(—CH₃)-aryl, C(═O)—(C₁-C₆)-alkyl, —C(═O)-cycloalkyl, —C(═O)—NH-alkyl, —C(═O)NH₂, hydroxy, —COOH (and ester thereof), alkylsulfonyl, arylsulfonyl, sulfonamide, amino, —NR′R″, —NHSO₂R₁, —NHC(═O)-alkyl —NH(C═0)NR′R″, trifluoromethyl, bromo, chloro, fluoro, iodo, cyclopropylmethyl, sulfonylmethyl; 3-6-membered cycloalkyl; 3-6-membered heterocycloalkyl, 5-7 membered aryl, or 5-7 membered heteroaryl, any of which may have one or more substituents listed herein, wherein the 3-6 membered heterocycloalkyl or 5-7 membered heteroaryls comprise at least one heteroatom independently selected from O, S, and N;
B is selected from the group consisting of a monocyclic, bicyclic, or spirocyclic cycloalkyl ring, and a monocyclic, bicyclic, or spirocyclic cycloheteroalkyl ring, wherein the cycloheteroalkyl contains 1-4 heteroatoms independently selected from N, O, and S, and wherein one or more carbons of the cycloalkyl or cycloheteroalkyl ring may be optionally substituted with one or more halogens or one or more haloalkyl groups;
C is selected from the group consisting of aryl, heteroaryl, heterocyclyl, cycloalkyl, and bridged cycloalkyl, wherein said aryl, heteroaryl, heterocyclyl, and bridged cycloalkyl comprises one or more substitutions (in addition to the R₂ group) selected from a group consisting of H, halo, alkyl, cyano, haloalkyl, and nitro;
R₁ is selected from H and C₁-C₃ alkyl;
R₂ is Formula (II): 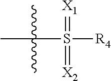
wherein:
X₁ is O and X₂ is NH or NR′;
X₁ is O and X₂ is NR₃; or
X₁ and X₂ are independently selected from NH or NR′;
wherein,
R₃ is independently selected from —(C═O)—(CH₂)ₙRₐ, —(C═O)—(CH₂)ₙ—(OCH₂CH₂)ₙRₐ, —(C═O)—(CH₂)ₙ—(OCH₂CH₂O)ₙRₐ, —(C═O)—(CH₂)ₙ—(NHCH₂CH₂)ₙRₐ, —(C═O)—(CH₂)ₙ—(NHCH₂CH₂O)ₙRₐ, —(CH₂)ₙRₐ, —(CH₂)ₙ(CR_{b}R_{c})Rₐ, —(C═O)—(NHCH₂CH₂NH)ₙRₐ, —(C═O)-(CH R_{b})Rₐ, —(CH₂)ₙ(CR_{b}R_{c}) (CH₂)ₙRₐ, or —(C═O)—(CR_{b}R_{c})Rₐ;
Rₐ is H, C₁-C₆-alkyl, branched alkyl, cycloakyl, aryl, heteroaryl, alkenyl, alkynyl, haloalkyl, alkoxy, cycloalkoxy, haloalkoxy, —CF₂-cycloalky, —CH(CH₃)-cycloalkyl, —CF₂-aryl, —NH₂, —NR′R″, amino cycloalkyl, 4 to 7 member heterocyclyl, —OH, —COONH₂, —COOH (and ester thereof), alkylsulfonyl, arylsulfonyl, sulfonamide, or amino;
R_{b} is selected from F, C₁-C₆-alkyl, haloalkyl, branched alkyl, aryl, heteroaryl, 3-7 membered carbocylyl, 4-7 membered heterocyclyl with one or more heteroatoms;
R_{c} is C₁-C₆-alkyl or F;
R_{c} R_{b} together with the carbon atom to which attached form a 3-6 membered carbocyclic ring or 4-6 membered heterocyclic ring with one or more hetero atoms;
R_{b} is selected from F, C₁-C₆-alkyl, haloalkyl, branched alkyl, aryl, heteroaryl, 3-7 membered carbocylyl, 4-7 membered heterocyclyl with one or more heteroatoms;
R_{c} is C₁-C₆-alkyl or F;
R_{c} R_{b} together with the carbon atom to which attached form a 3-6 membered carbocyclic ring or 4-6 member heterocyclic ring with one or more hetero atoms;
R₄ is selected from C₁-C₃ alkyl, C₃-C₄ cycloalkyl, haloalkyl, halocycloalkyl, aryl, heteroaryl, or heterocyclyl; and
n=0, 1,2,3,4,5,6.

2. The compound of claim 1, wherein A is an optionally substituted aryl, an optionally substituted heteroaryl an optionally substituted 5 or 6 members heteroaryl with one or more heteroatom; wherein the heteroaryl group contains a nitrogen atom in a ring, such nitrogen atom may be in the form of an N-oxide, wherein A is optionally a pyridyl N-oxide, pyrazinyl N-oxide, pyrimidinyl N-oxide, and pyridazinyl N-oxide.

3. The compound of claim 1, wherein A contains a nitrogen atom in the heteroaryl ring, such nitrogen atom may be in the form of an N-oxide selected from a group consisting of pyridyl N-oxide, pyrazinyl N-oxide, pyrimidinyl N-oxide, and pyridazinyl N-oxide, and wherein A may be substituted with one or more substituents.

4. The compound of claim 1, wherein A is an optionally substituted aryl.

5. The compound of claim 1, wherein A is an optionally substituted heteroaryl with one heteroatom.

6. The compound of claim 1, wherein A is an optionally substituted heteroaryl with two heteroatoms.

7. The compound of claim 1, wherein the heteroaryl A is substituted with at least triflouoromethyl.

8. The compound of claim 1, wherein the heteroaryl ring of A is substituted with at least cyclopropylmethyl.

9. The compound of claim 1, wherein A is represented by the following Formula(s): 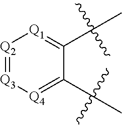
wherein each of Q₁, Q₂, Q₃ and Q₄ is independently N, N—O, or CR₅;
wherein R₅ is H, hydroxyl, halogen, —CD₃, alkyl, haloalkyl, alkoxy, haloalkoxy, alkyl sulfonyl, alkyl sulfoximinyl, alkyl sulfonamide, cyano, —CF₃, —OCF₃, heterocyclyl in which each ring has 4 to 6 members, heteroaryl having a 5 or 6 ring membered, saturated heterocyclyl, or partially unsaturated heterocyclyl, O-aryl in which each ring has 5 or 6 members, O-heteroaryl in which each ring has 5 or 6 members, O-cycloalkyl, O— cycloheteroalkyl, each of which is optionally substituted.

10. The compound of claim 1, wherein A is an optionally substituted 5 or more membered saturated or partially unsaturated heterocyclic ring having one or two heteroatoms independently selected from N, O, or S.

11. The compound of claim 1, wherein, A is represented the following Formula(s): 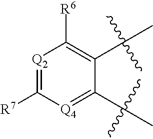
wherein:
Q₂ and Q₄ is N, N—O;
Q₂ is N, N—O; Q₄ is CR₅;
Q₂ is CR₅; Q₄ is N, N—O;
R₆ is H, halogen, —CD₃, C₁-C₆ alkyl, branched alkyl, alkenyl, alkynyl, haloalkyl, alkoxy, cycloalkyl, heterocyclyl, NH₂, NHR′, NR′R″, NHC(═O)R′, NHSO₂R, —C(═O)R′, —C(═O)NHR′, —C(═O)NR′R″, aryl, Heteroaryl, —CF₂CH₃, —CF₂CF₃;
R₇ is H, hydroxyl group, halogen, —CD₃, C₁-C₆ alkyl, branched alkyl, allyl, alkenyl, alkynyl, haloalkyl, alkoxy, cycloalkoxy, haloalkoxy, nitro, cyano, —CH₂-cycloalkyl, —CF₂CH₃, —CF₂CF₃, CH₂CF₂, —CF₂-cycloalky, —CH(CH₃)-cycloalkyl, —CH₂-aryl, —CF₂-heteroaryl, —CF₂-heterocyclyl, —CH(—CH₃)-aryl, C(═O)-alkyl, —C(═O)cycloalkyl, —C(═O)—NH-alkyl, —C(═O)NH₂, —C(═O)NHR′, —C(═O)NR′R″, hydroxy, —COOH (and ester thereof), alkylsulfonyl, arylsulfonyl, sulfonamide, amino, NR′R″, —NHSO₂R′, —NHC(═O)-alkyl —NH(C═O)NR′R″, trifluoromethyl, cyclopropylmethyl, methylsulfonyl, 3-6 membered cycloalkyl; 3-6 membered heterocycloalkyl, any of which may have one or more substitutents, wherein the 3-6 membered heterocycloalkyl comprises at least one heteroatom independently selected from O, S, and N;
wherein not more than two of Q₁, Q₂, Q₃ and Q₄ or N or N-0.

12. The compound of claim 1, wherein, A is represented the following Formula(s): 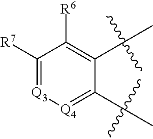
wherein:
Q₃ and Q₄ are N; wherein Q₃ is N, N—O; Q₄ is CR₅; or wherein Q₃ is CR₅, Q₄ is N, N—O;
R₅ is defined in claim 9; and
R₆ and R₇ are defined above in claim 11.

13. The compound of claim 1, wherein, A is represented the following Formula(s): 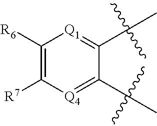
wherein Q₁ and Q₄ are N, N—O; Q₁ is N, N—O; Q₄ is CR₅; Q₁ is CR₅, Q₄ is N, N—O; or Q₁ and Q₄ are CR₅; and wherein R₆ and R₇ are defined above in claim 11.

14. The compound of claim 1, wherein, A is represented the following Formula(s): 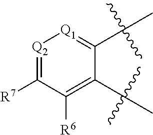
wherein:
Q₁ and Q₂ are N; and
R₆ and R₇ are defined above in claim 11.

15. The compound of claim 1, wherein, A is represented the following Formula(s): 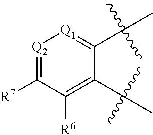
wherein:
Q₁ is CR₅, and Q₂ is N;
R₆ and R₇ are defined above in claim 11.

16. The compound of claim 1, wherein, A is represented the following Formula(s): 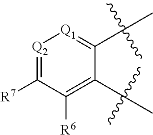
wherein Q₁ is N; and Q₂ is CR₅; and R₆ and R₇ are defined above in claim 11.

17. The compound of claim 1, wherein the heteroaryl ring of A is selected from the group consisting of:

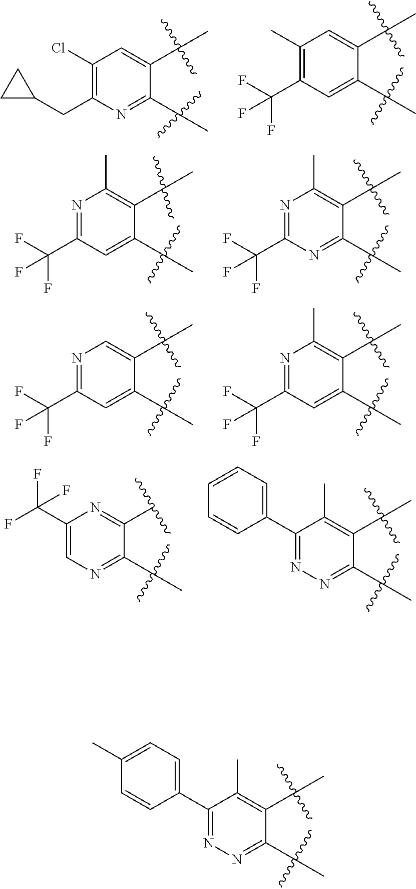

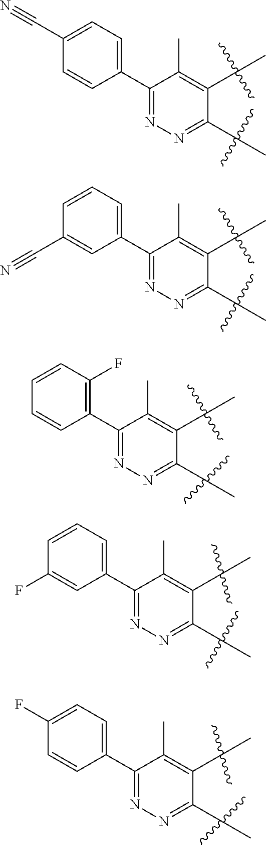 [SMILES: *c1nnc(-c2ccc(C#N)cc2)c(C)c1* ; *c1nnc(-c2cccc(C#N)c2)c(C)c1* ; *c1nnc(-c2ccccc2F)c(C)c1* ; *c1nnc(-c2cccc(F)c2)c(C)c1* ; *c1nnc(-c2ccc(F)cc2)c(C)c1*]

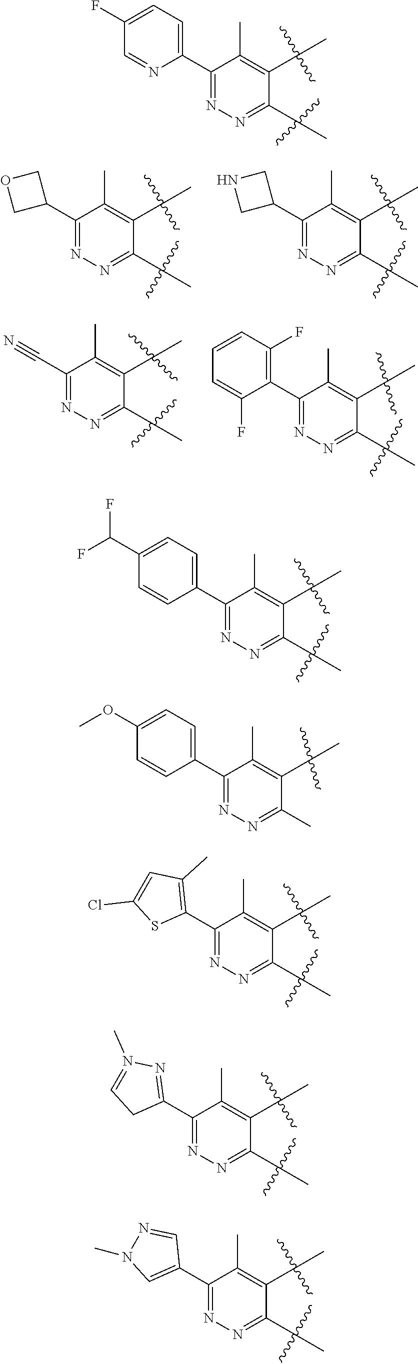

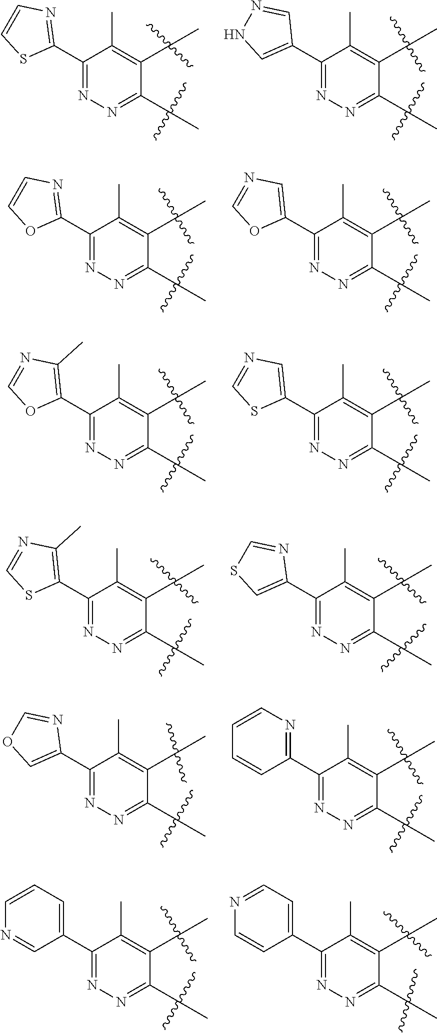

18. The compound of claim 1, wherein B is selected from the group consisting of pyrrolidine, azetidine, piperidine, piperazine, azepane, azocane, morpholine, thiomorpholine, oxazepane, isoindoline, dihydroisoquinoline, octahydroisoindole, azabicyclo[2.2.1]heptane, azabicyclo[3.1.1]heptane, azabicyclo[4.1.0]heptane, azabicyclo[3.2.1]octane, diazabicyclo-[3.2.1]octane, azabicyclo[3.2.0]heptane, oxa-azabicyclo[3.2.1]octane, azaspiro[2.5]octane, azaspiro[2.6]nonane, azaspiro[3.5]nonane, oxa-azaspiro[3.5]nonane, oxa-azaspiro[4.5]decane, dihydrothieno[3,2-c]pyridine, dihydrothiazolo[4,5-c]pyridine, dihydrooxazolo[4,5-c]pyridine, dihydroimidazo[1,2-a]pyrazine, hexahydrofuro[3,2-b]pyrrole, hexahydrocyclopenta[c]pyrrole, and azatricyclo[4.3.1.13,8]undecane.

19. The compound of claim 1, wherein C is phenyl.

20. The compound of claim 1, wherein C is pyridyl.

21. The compound of claim 1, wherein within Formula (II), X₁ is O and X₂ is NH.

22. The compound of claim 1, wherein the compound of Formula (I) is further described as Formula (III): 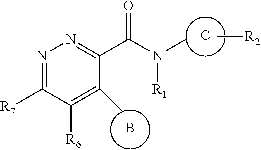
wherein:
A, B, C, R₁, and R₂ are defined above in claim 1, and R₆ and R₇ are defined above in claim 11.

23. The compound of claim 1, wherein substituted or unsubstituted B is selected from a group consisting of:

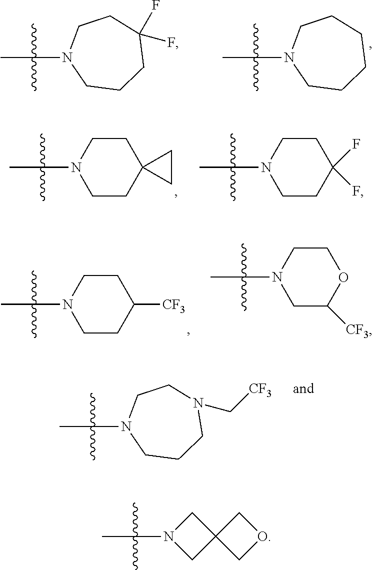

24. The compound of claim 1, wherein C is selected from a group consisting of:

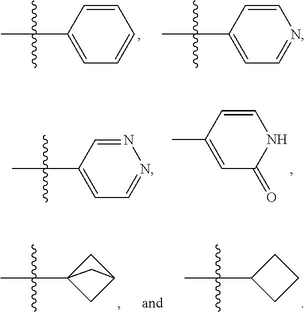

25. The compound of claim 1, wherein R₁ is H.

26. The compound of claim 1, wherein R₂ is selected from a group consisting of:

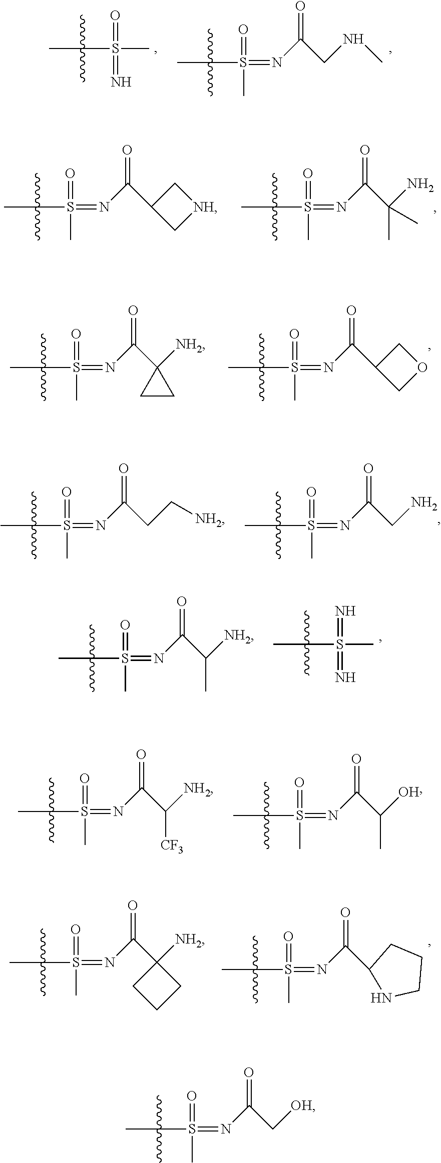

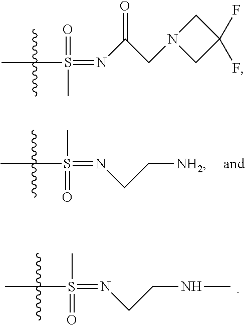

27. The compound of claim 1, wherein R₂ is:

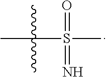 [SMILES: *S(C)(=N)=O]

28. The compound of claim 1, wherein C comprises additional substitutions, wherein the substitutions are selected from a group consisting of H, halo, and alkyl.

29. The compound of claim 28, wherein the halo is F.

30. The compound of claim 1, wherein the one or more substitutions on the A ring are selected from a group consisting of: halo, cyano, haloalkyl, cyanoalkyl, substituted or unsubstituted C₁-C₆ alkyl, aryl, C₃-C₆ cycloalkyl, C₃-C₆ heterocycloalkyl, C₃-C₆ heteroaryl, and any combination thereof, wherein the heterocycloalkyl and heteroaryl comprise one or more hetero atoms are selected from a N, O, or S.

31. The compound of claim 1, wherein the one or more substitutions on the A ring are selected from a group consisting of: methyl, trifluoromethyl, chloro, fluoro, bromo, C₁-C₆ alkyl, phenyl, cycloalkyl, methyl pyrazole, fused 1,4 dioxane, and methylcyano.

32. The compound of claim 1, wherein the one or more substitutions on the A ring are selected from a group consisting of: —CH₃, —CF₃, —Cl, —Br, —F, —CH₂—CH₂—CH═CH₂, phenyl, —CH₂—CN, —C(═O)—NH₂, 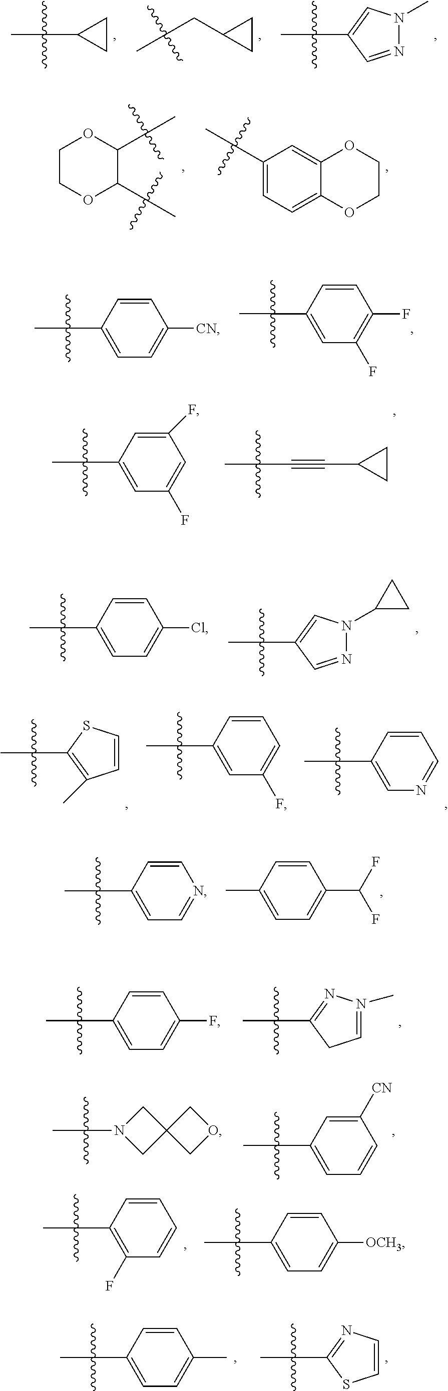 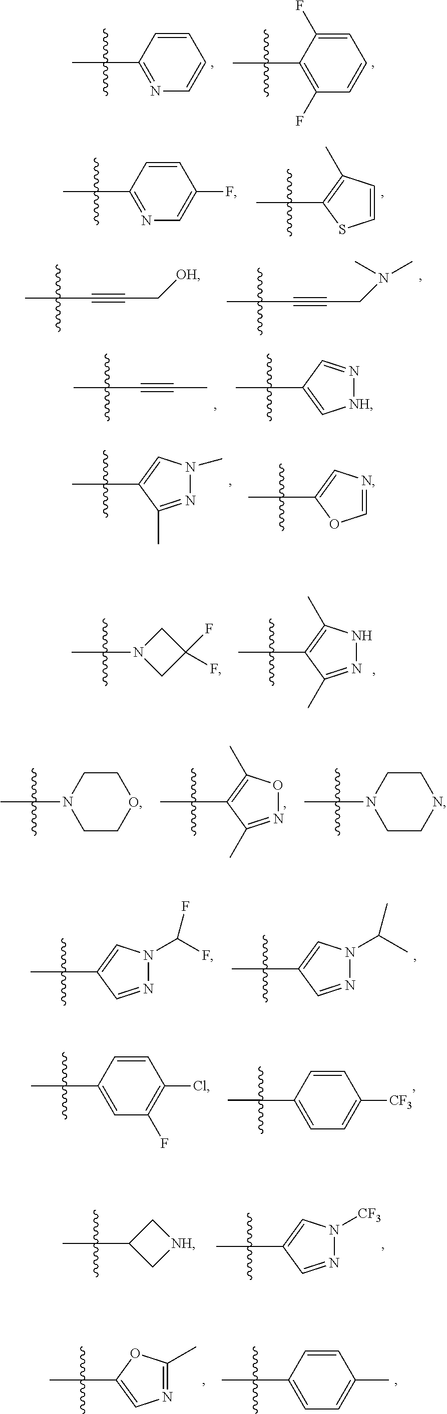
an any combination thereof.

33. A compound is selected from the group consisting of

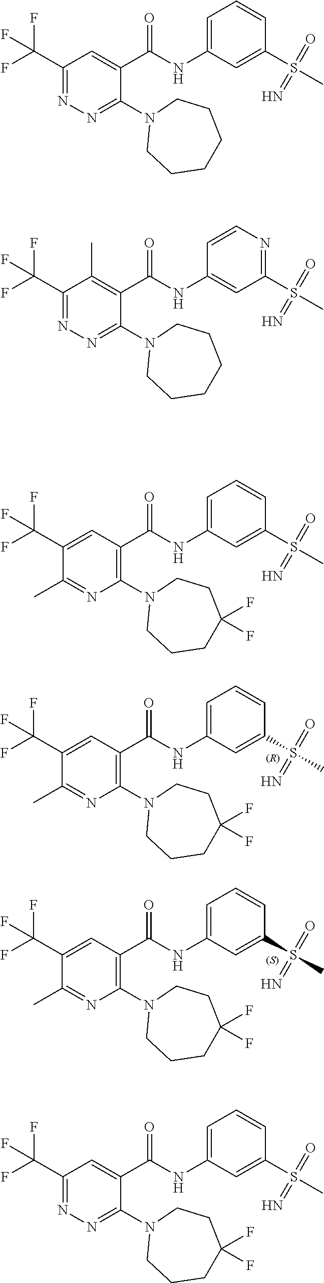

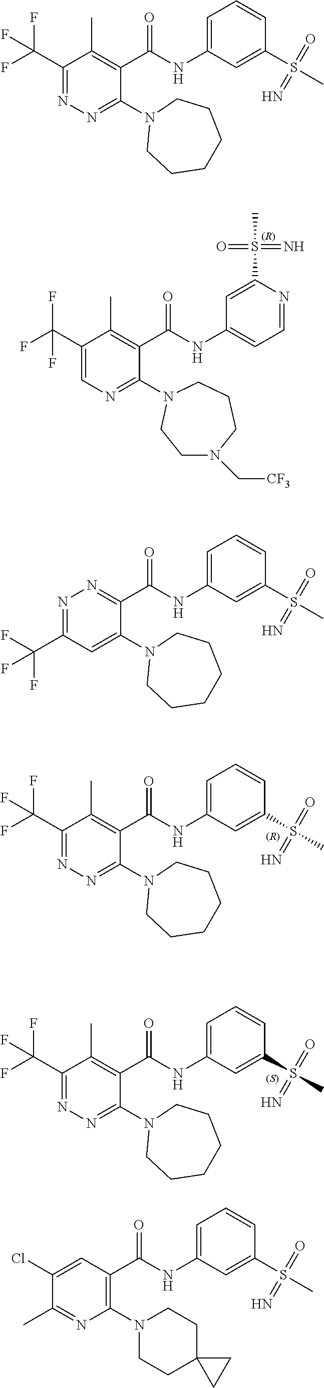

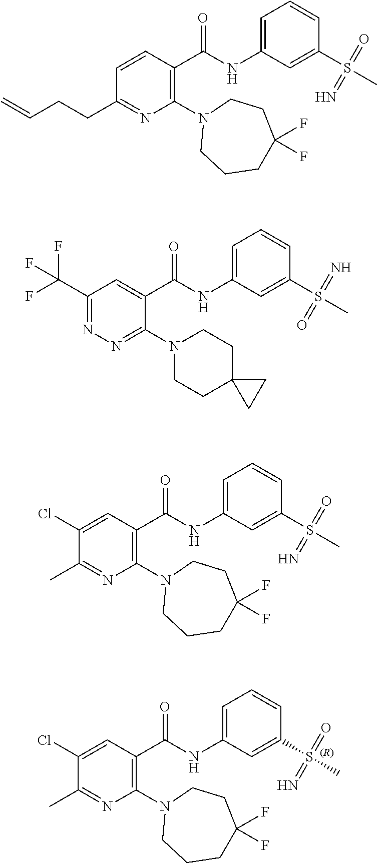

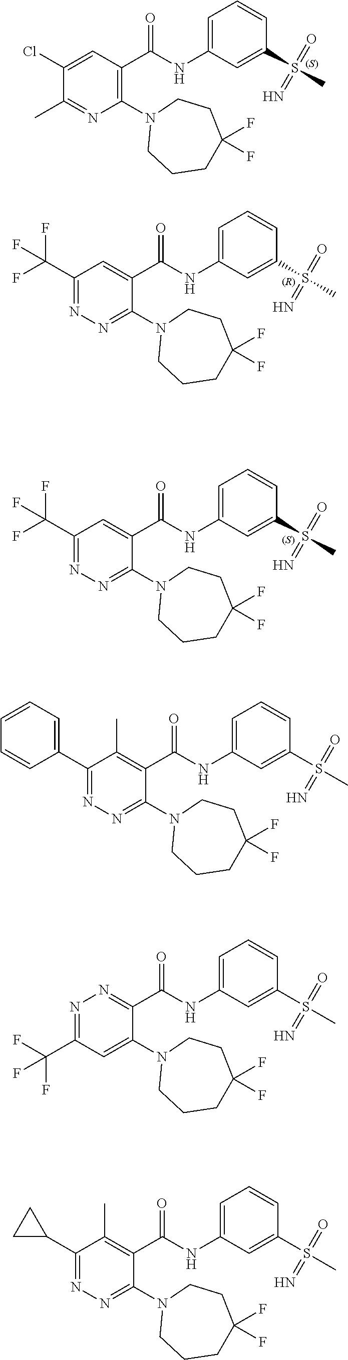

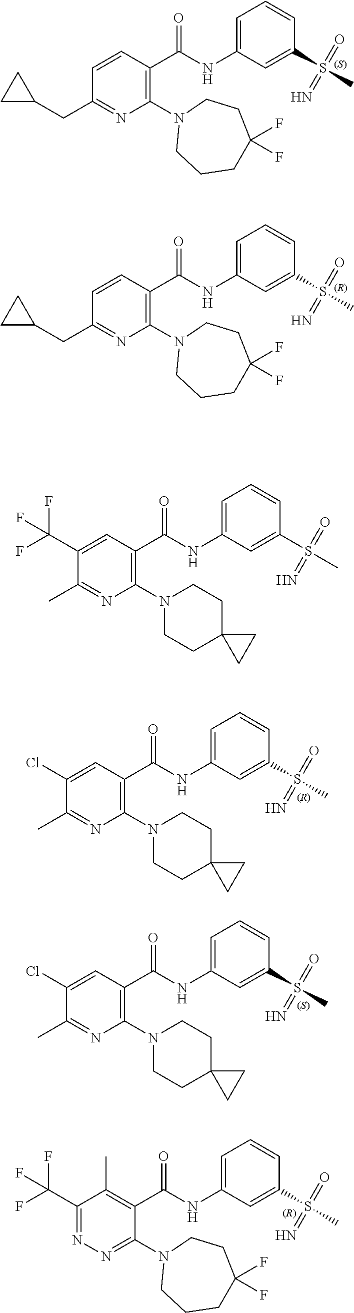

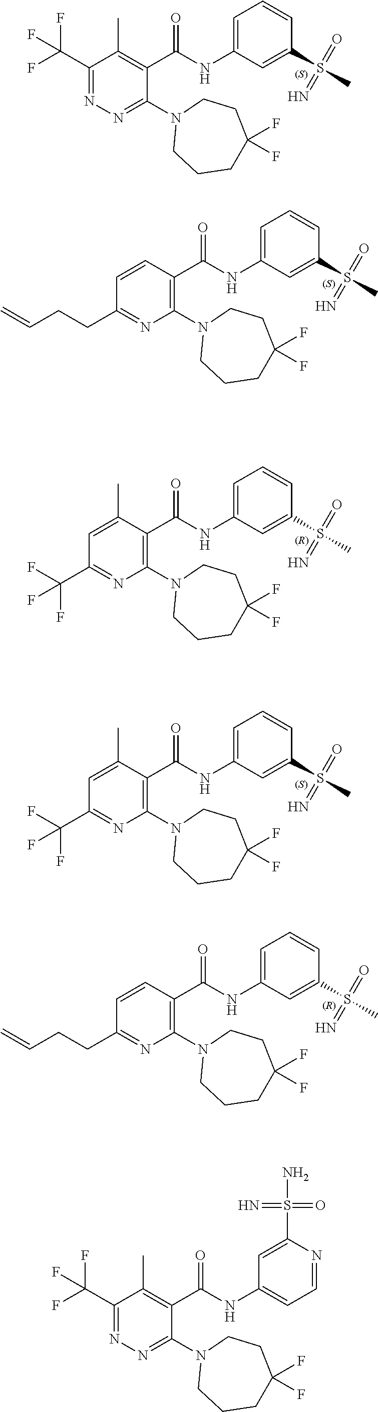

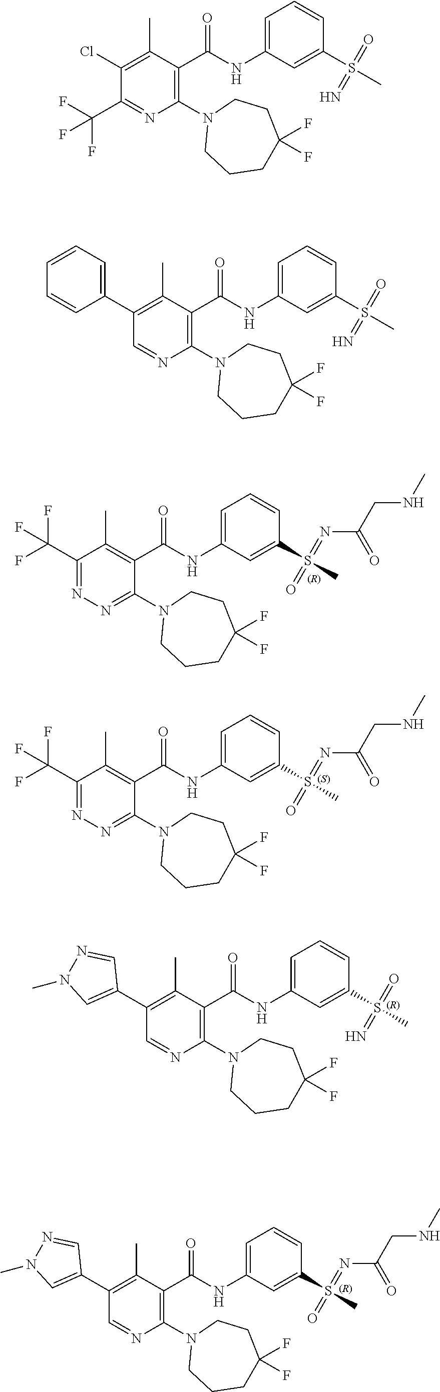

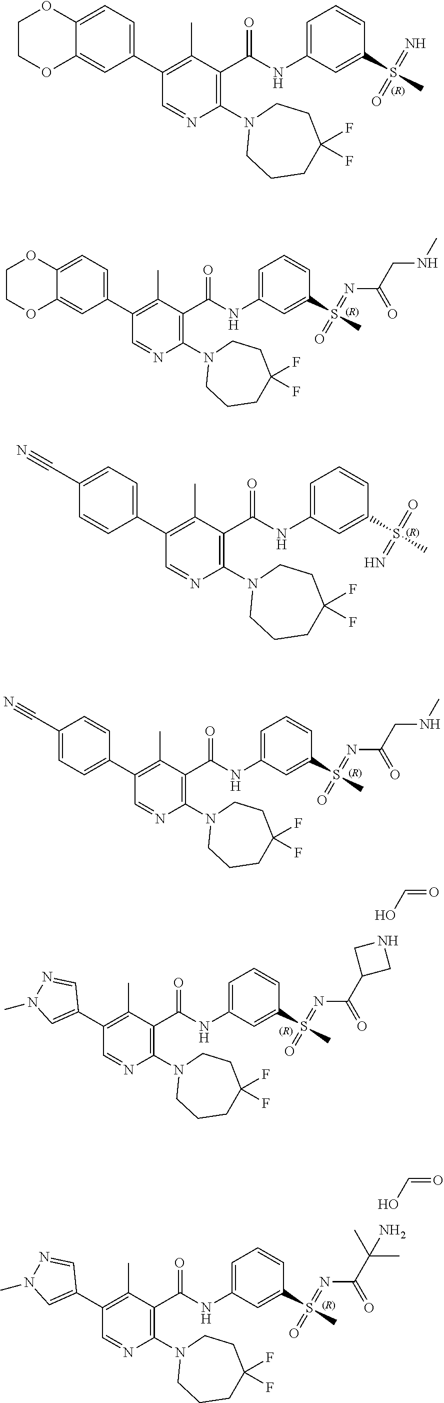

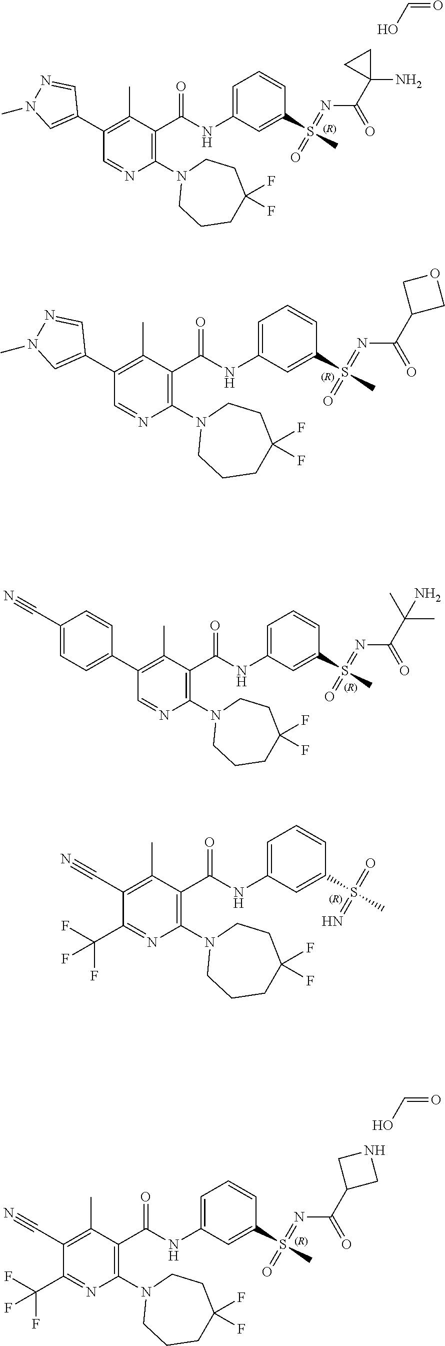

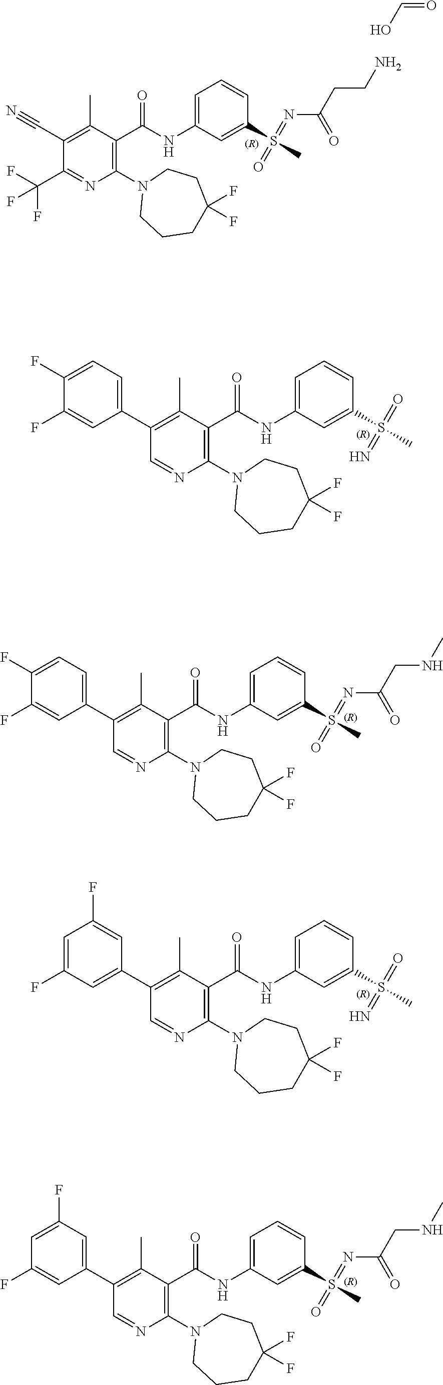

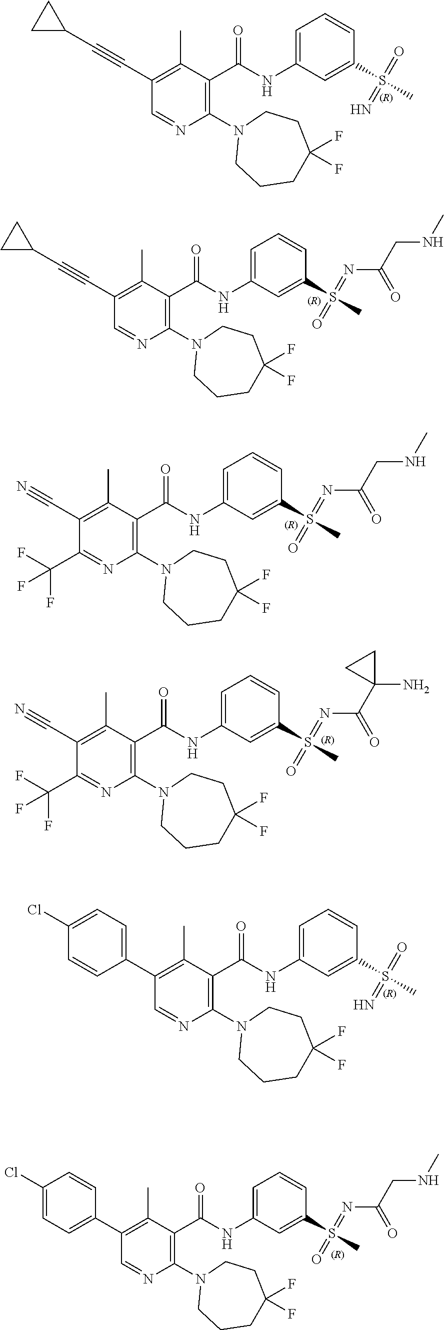

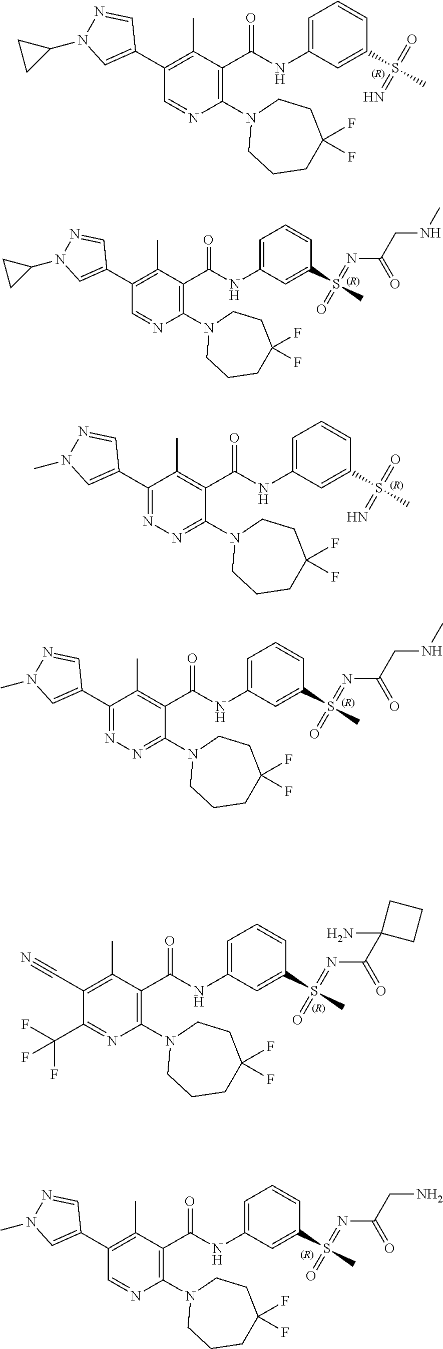

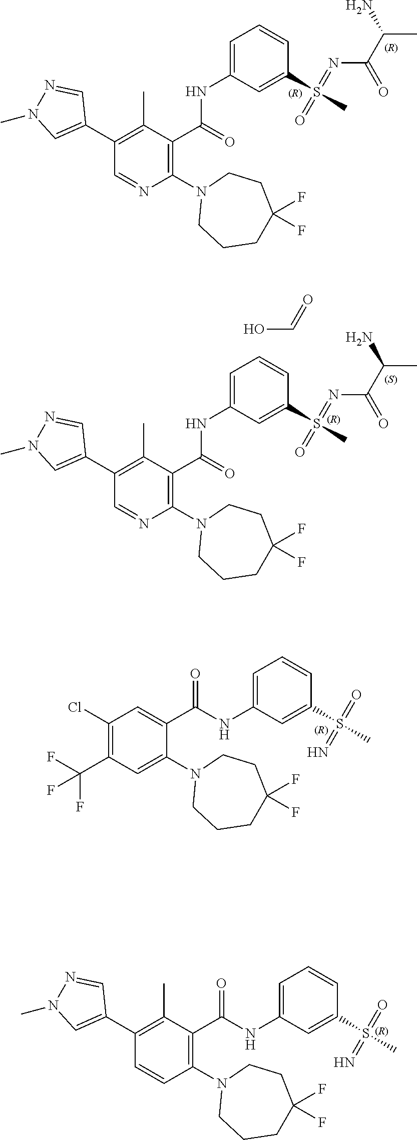

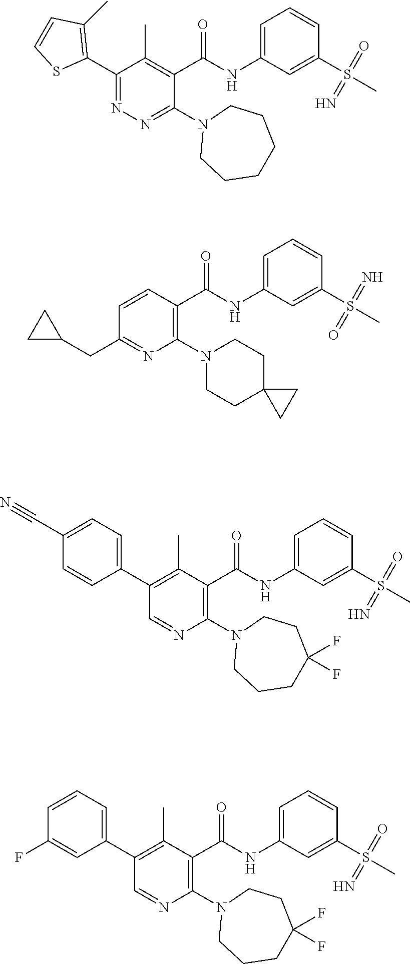 [SMILES: Cc1ccsc1-c1nnc(N2CCCCCC2)c(C(=O)Nc2cccc(S(C)(=N)=O)c2)c1C ; CS(=N)(=O)c1cccc(NC(=O)c2ccc(CC3CC3)nc2N2CCC3(CC2)CC3)c1 ; Cc1c(-c2ccc(C#N)cc2)cnc(N2CCCC(F)(F)CC2)c1C(=O)Nc1cccc(S(C)(=N)=O)c1 ; Cc1c(-c2cccc(F)c2)cnc(N2CCCC(F)(F)CC2)c1C(=O)Nc1cccc(S(C)(=N)=O)c1]

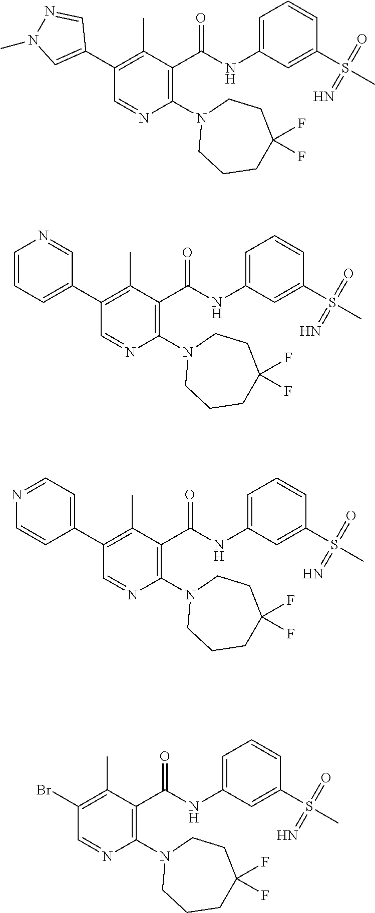

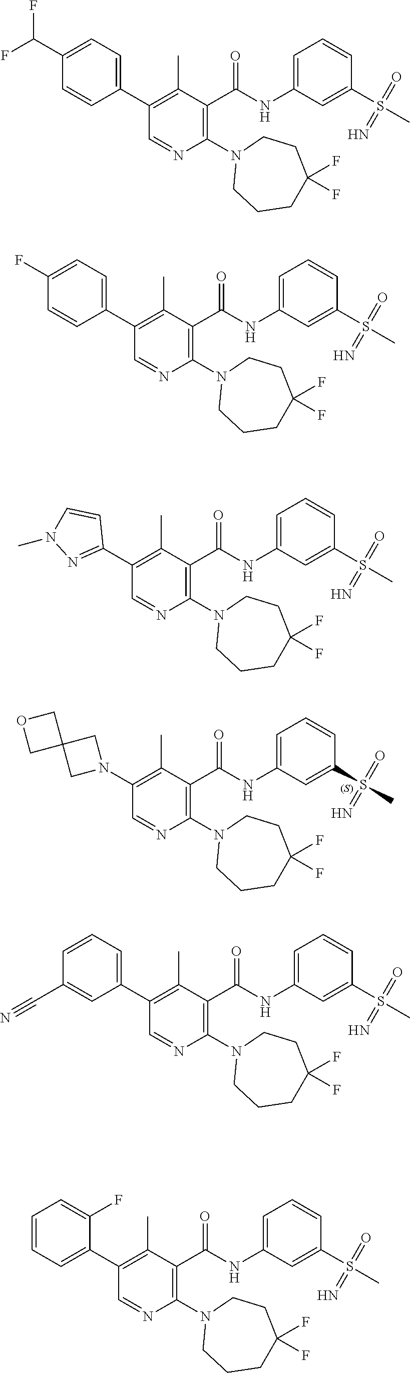

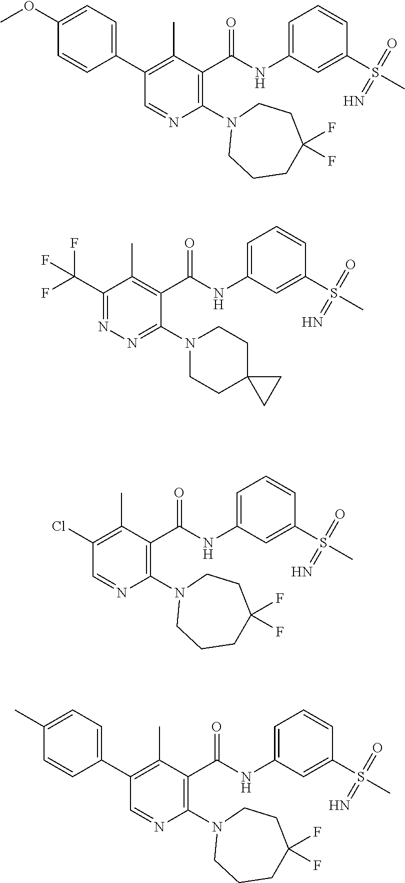

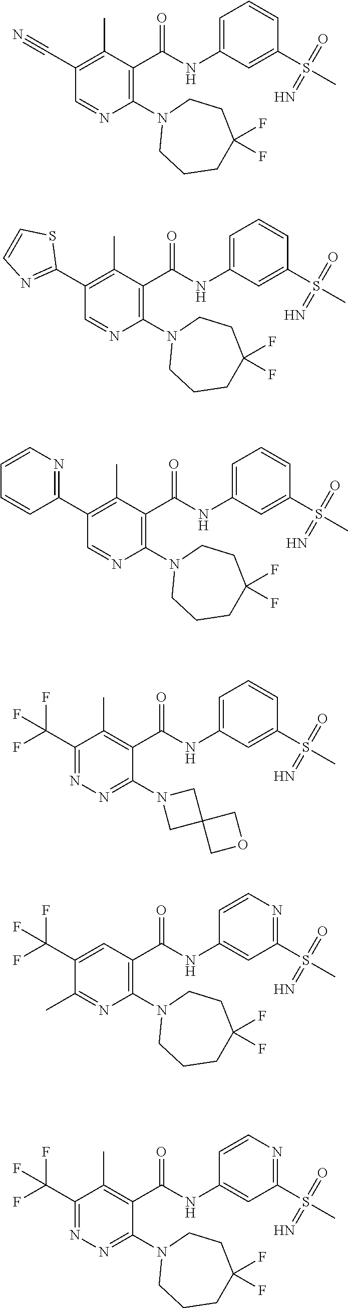 [SMILES: Cc1c(C#N)cnc(N2CCCC(F)(F)CC2)c1C(=O)Nc1cccc(S(C)(=N)=O)c1 ; Cc1c(-c2nccs2)cnc(N2CCCC(F)(F)CC2)c1C(=O)Nc1cccc(S(C)(=N)=O)c1 ; Cc1c(-c2ccccn2)cnc(N2CCCC(F)(F)CC2)c1C(=O)Nc1cccc(S(C)(=N)=O)c1 ; Cc1c(C(F)(F)F)nnc(N2CC3(COC3)C2)c1C(=O)Nc1cccc(S(C)(=N)=O)c1 ; Cc1nc(N2CCCC(F)(F)CC2)c(C(=O)Nc2ccnc(S(C)(=N)=O)c2)cc1C(F)(F)F ; Cc1c(C(F)(F)F)nnc(N2CCCC(F)(F)CC2)c1C(=O)Nc1ccnc(S(C)(=N)=O)c1]

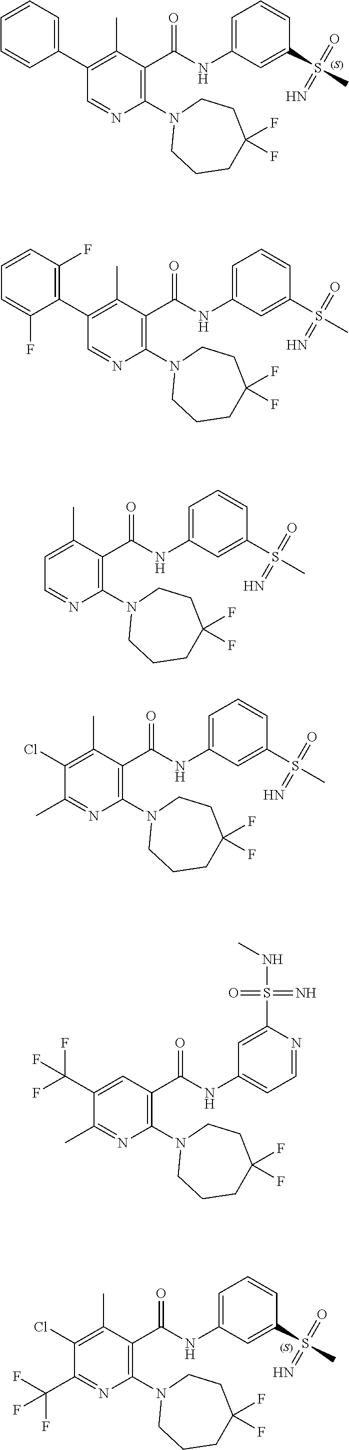

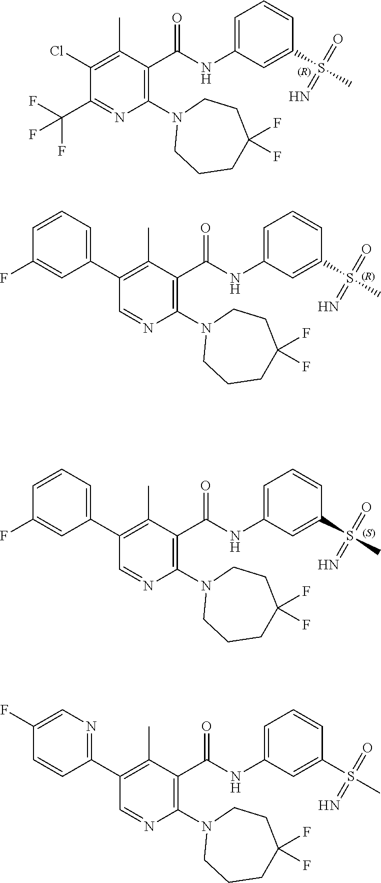

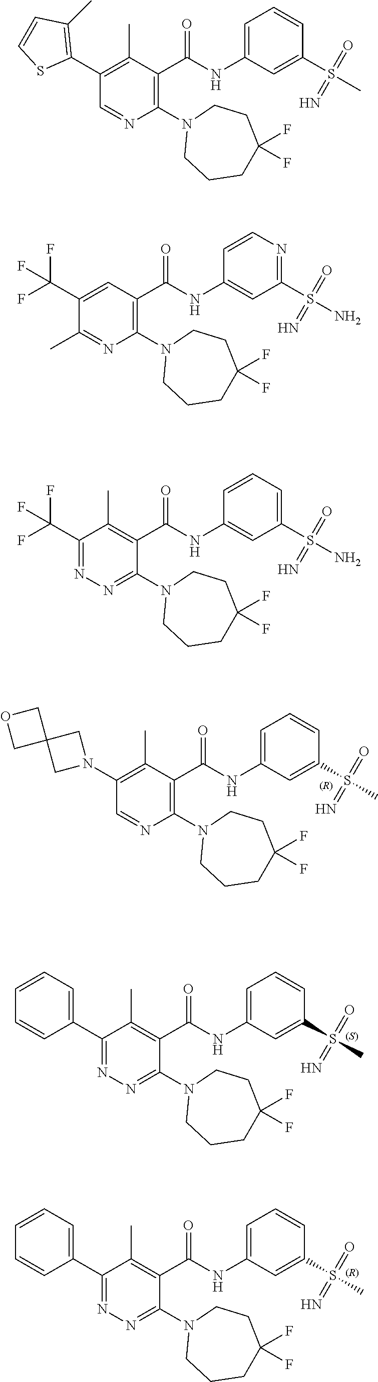

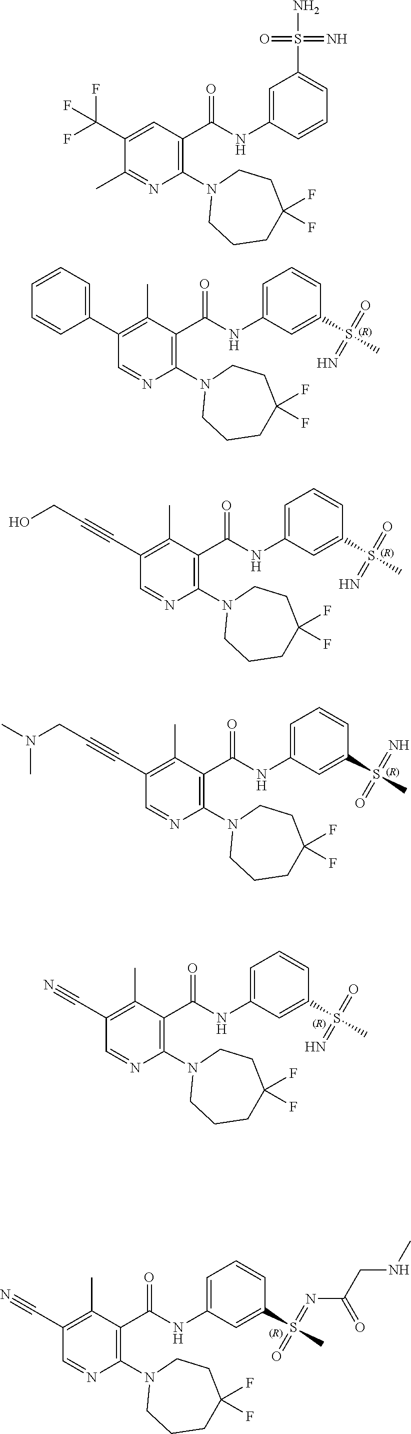

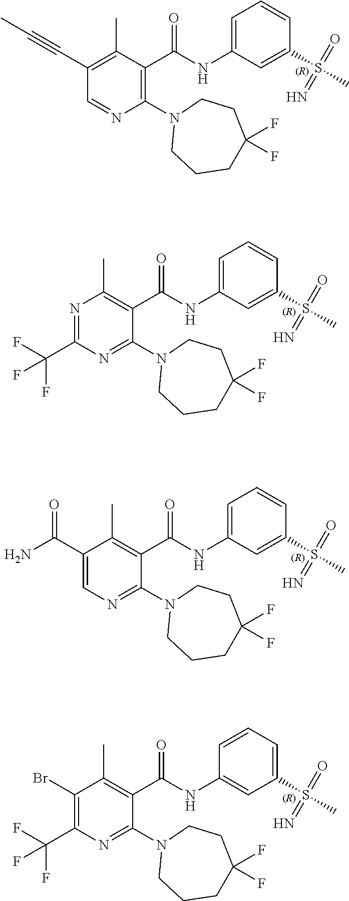

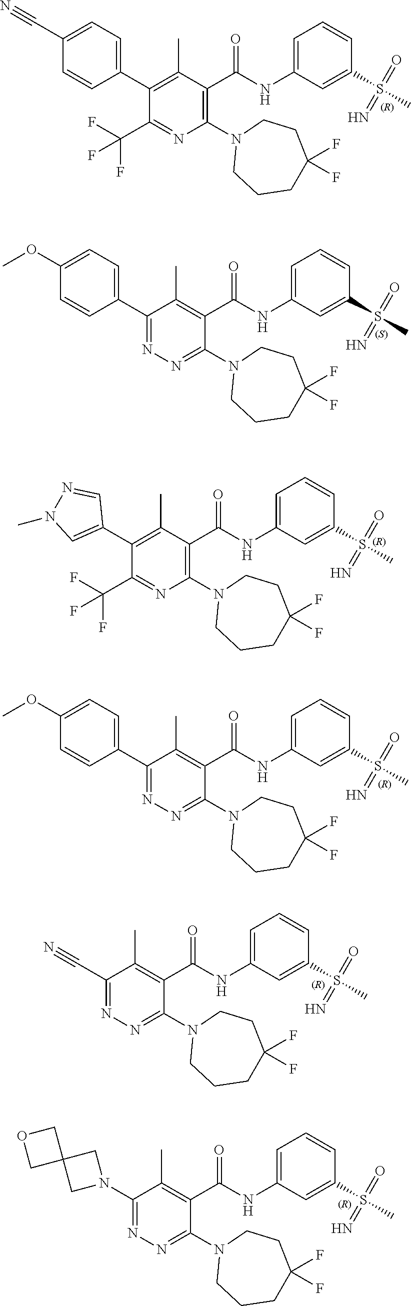

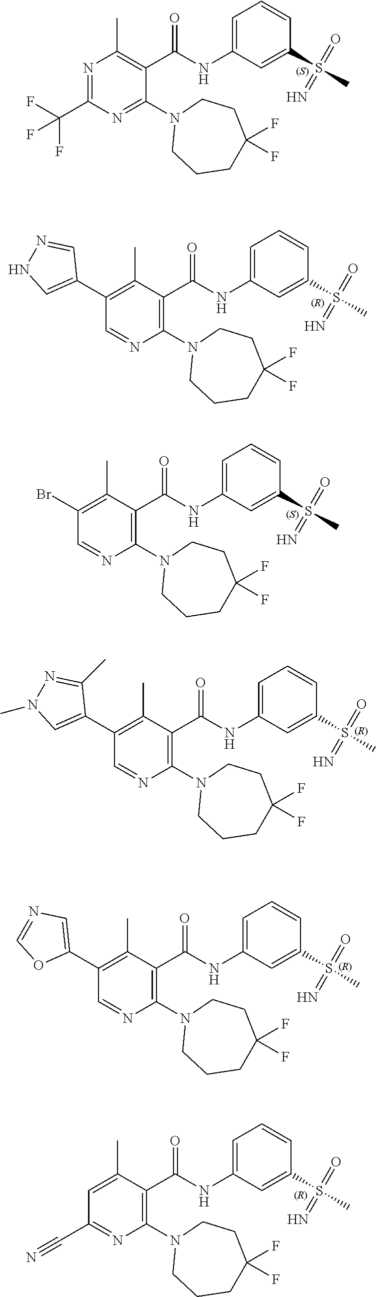

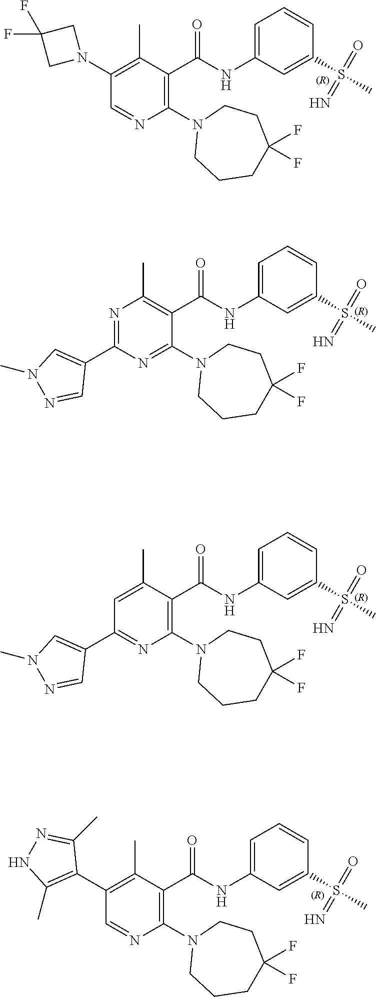

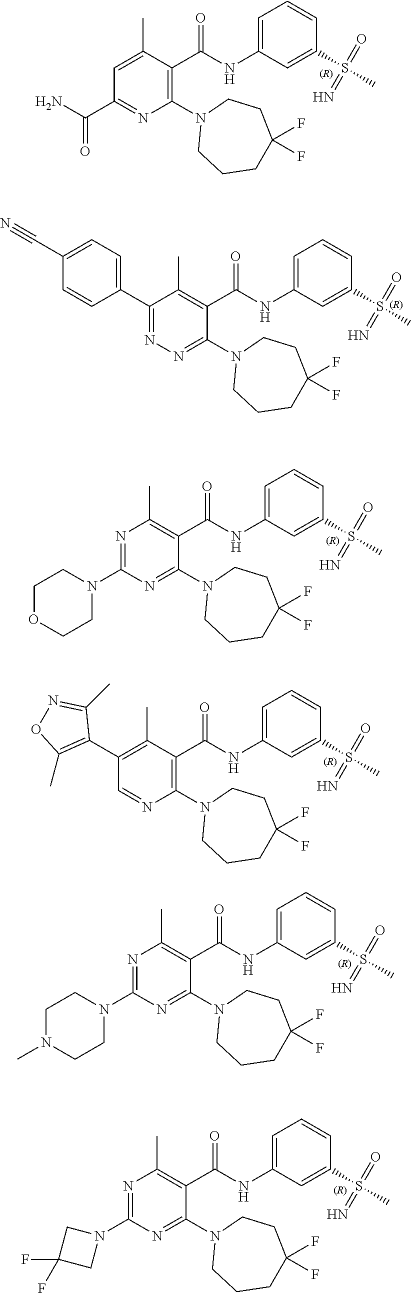

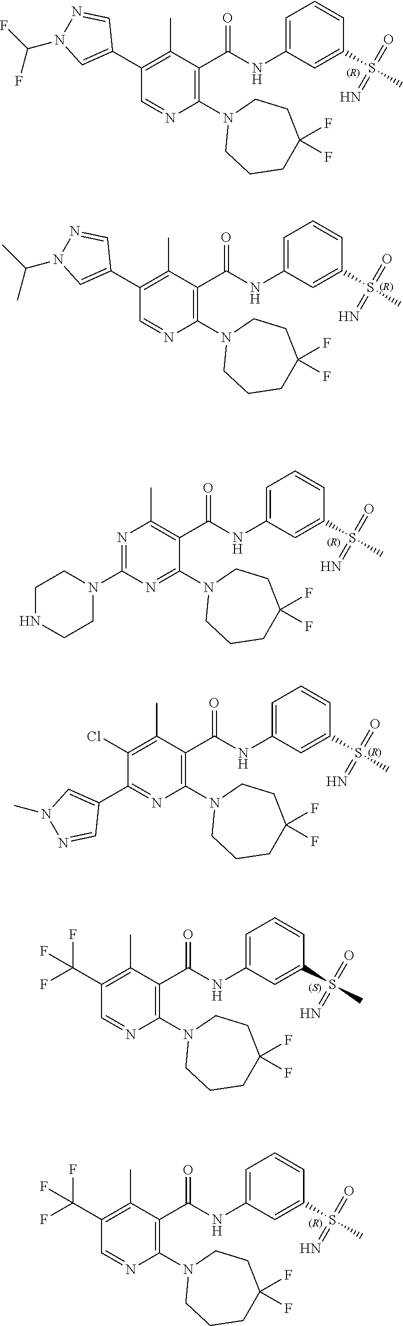

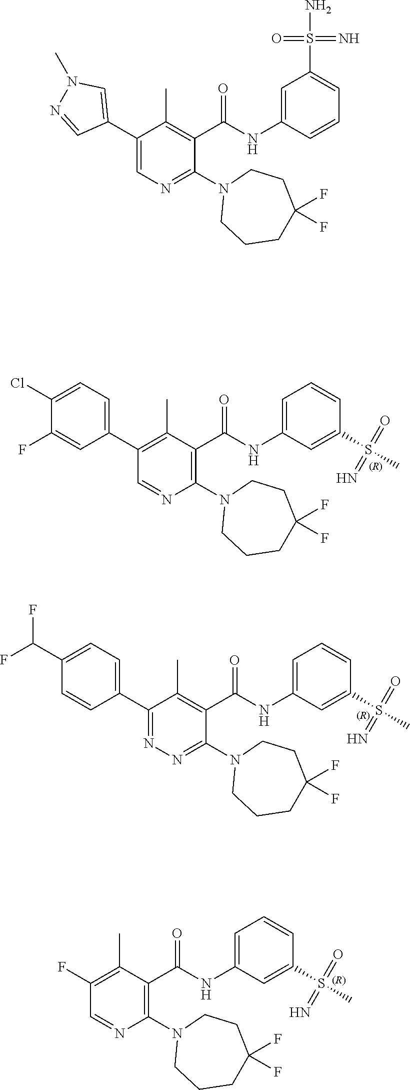

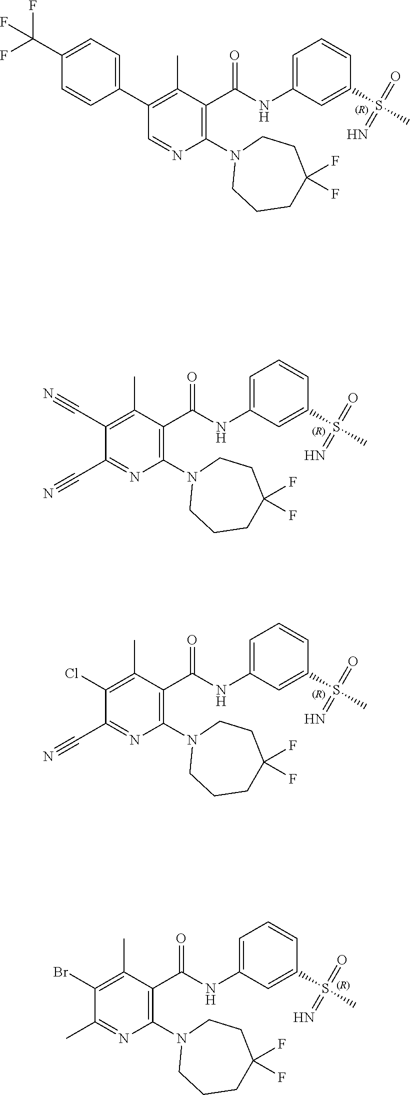

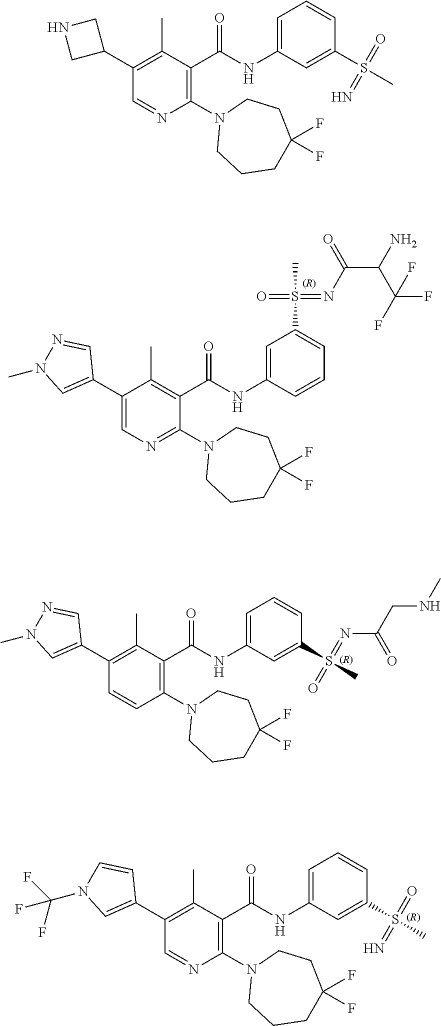

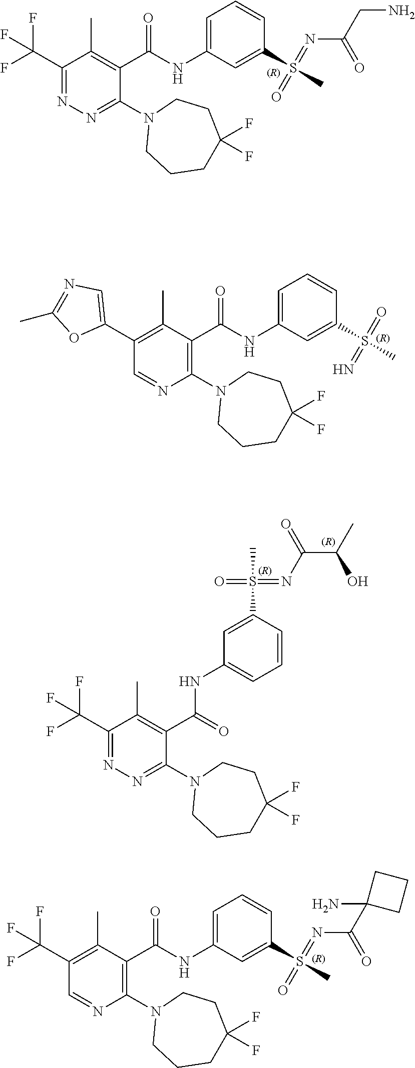

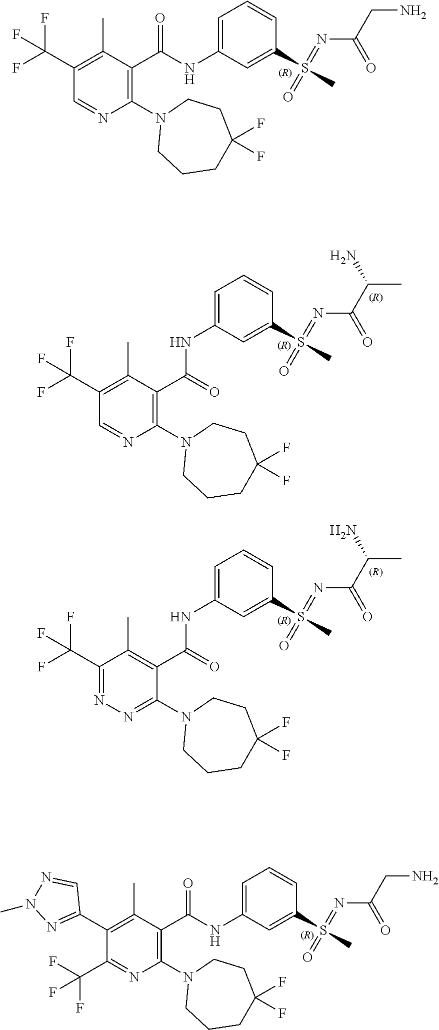

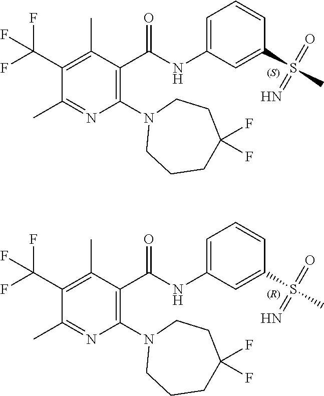

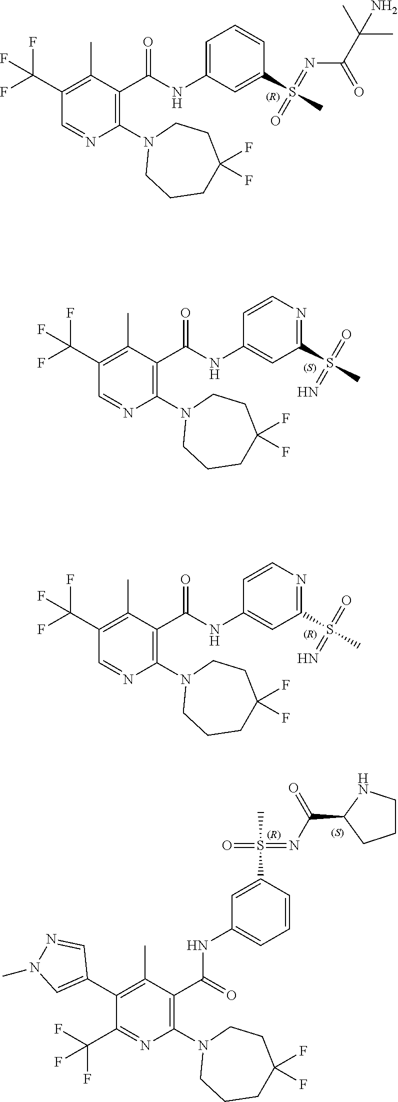

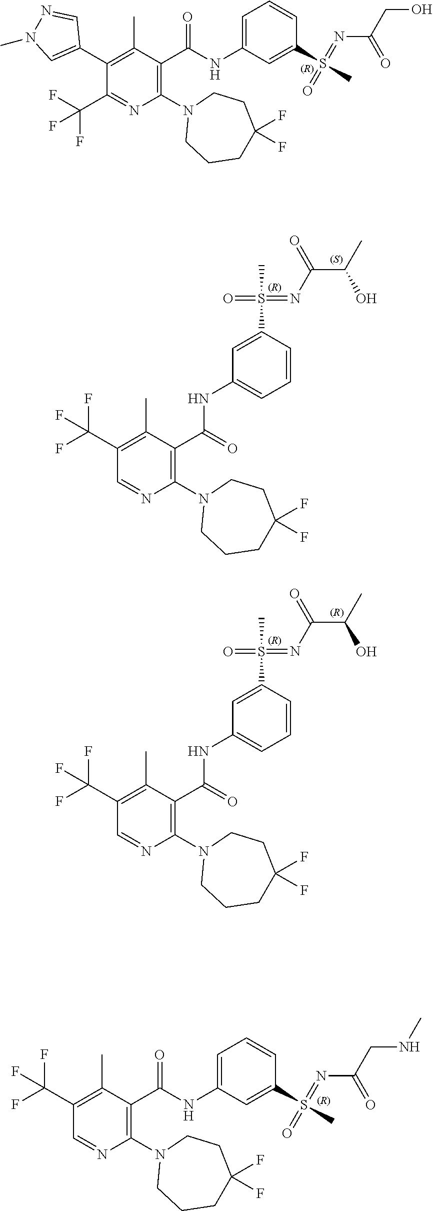

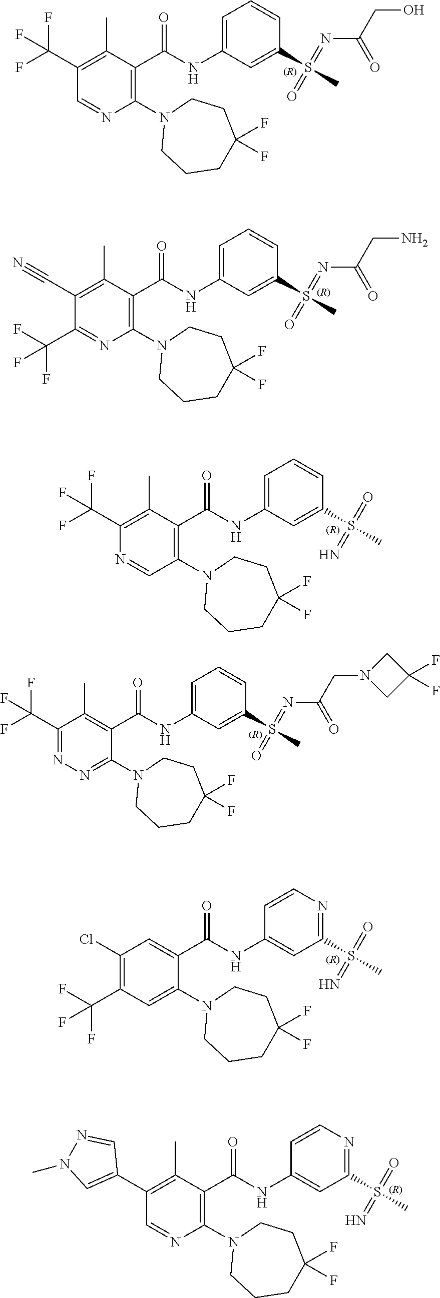

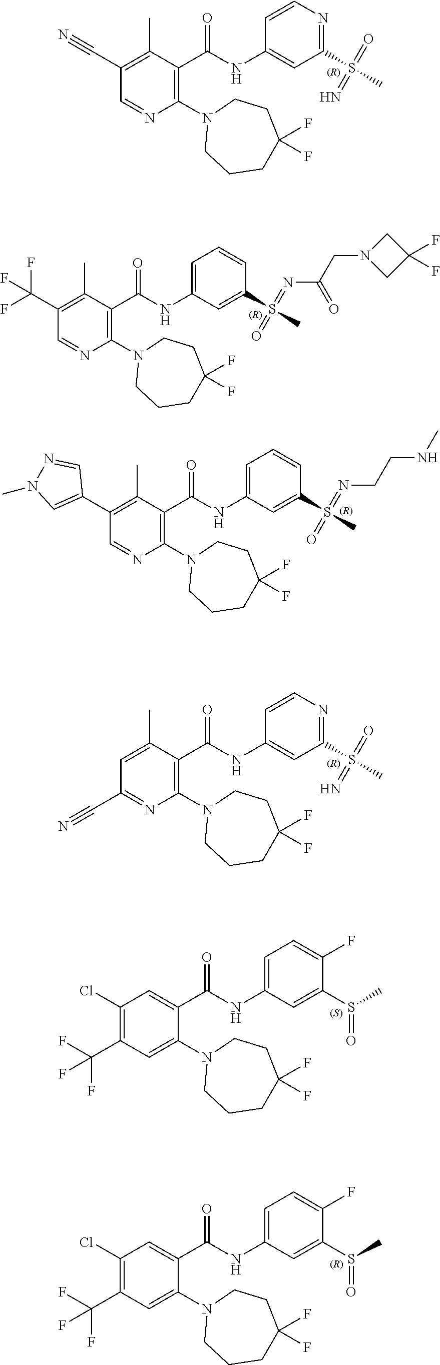

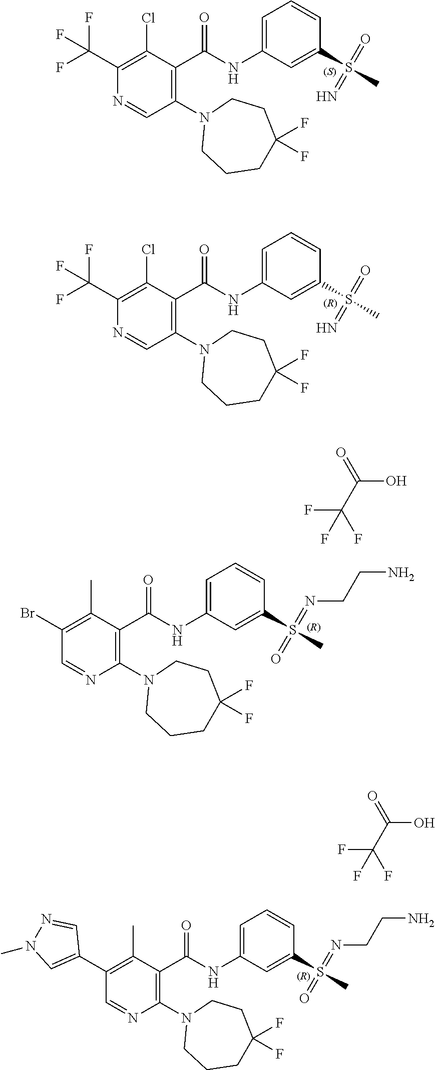

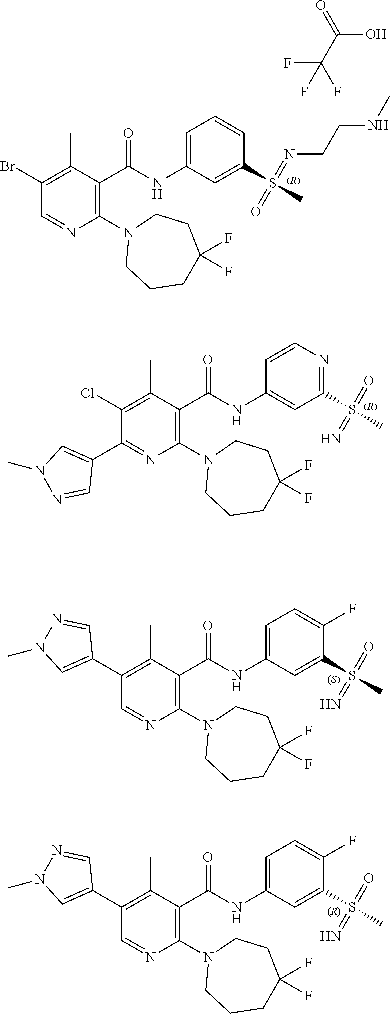

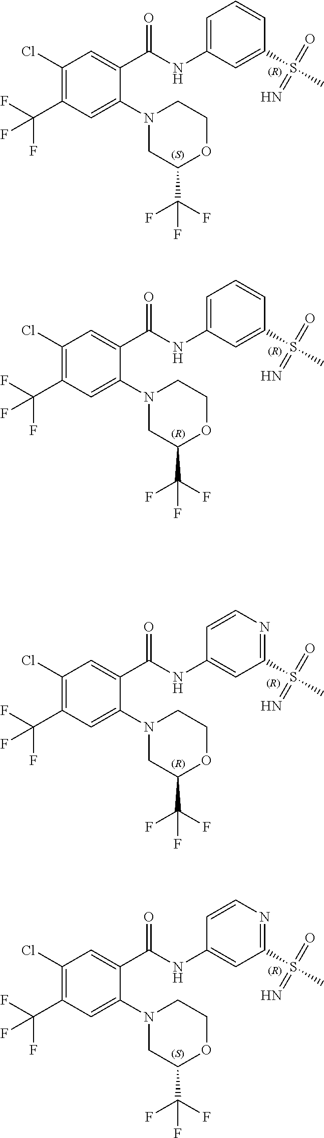

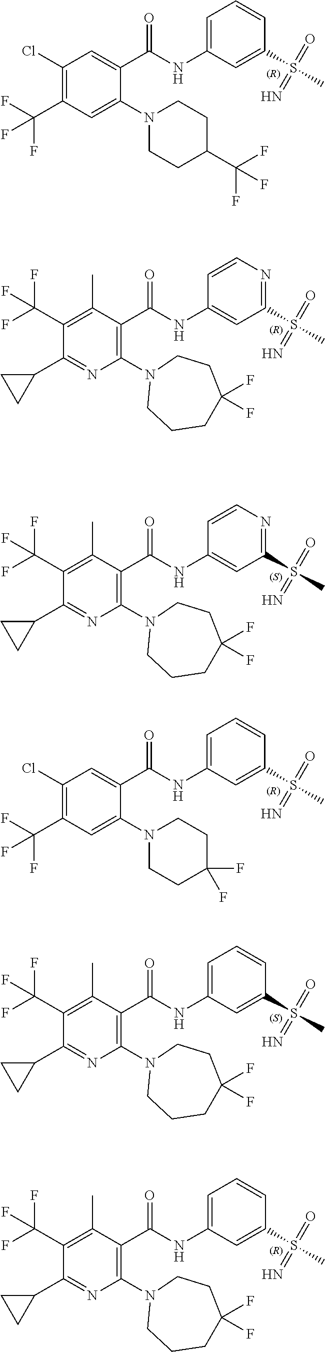

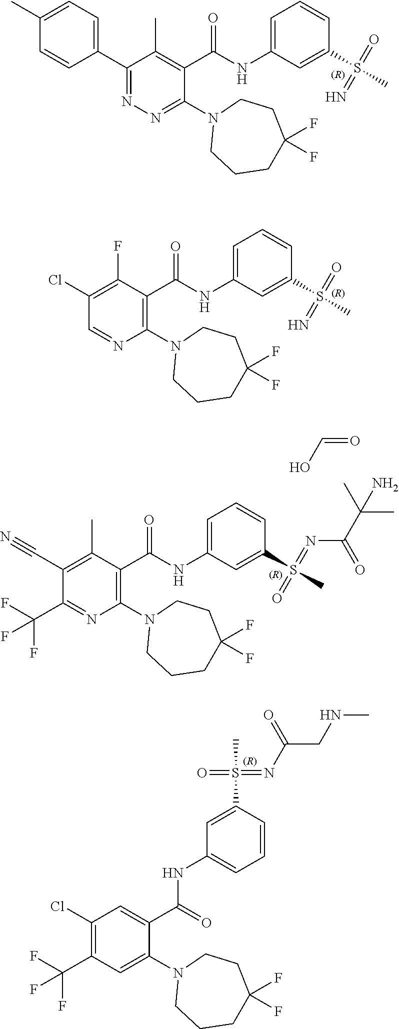

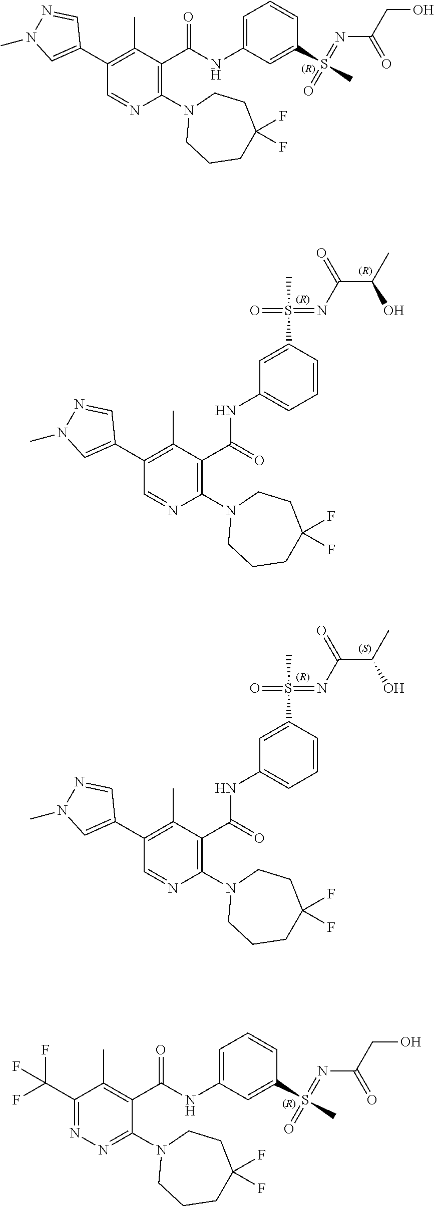

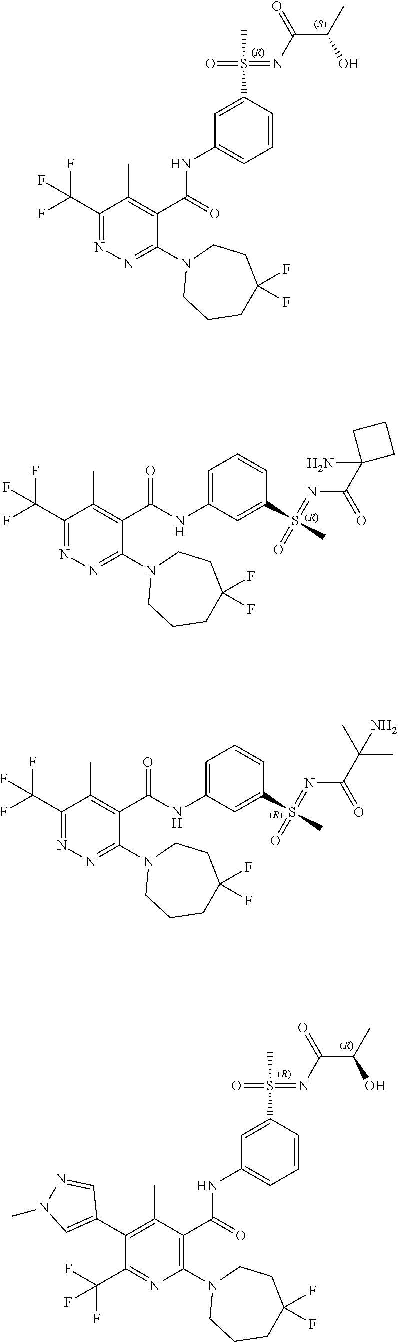

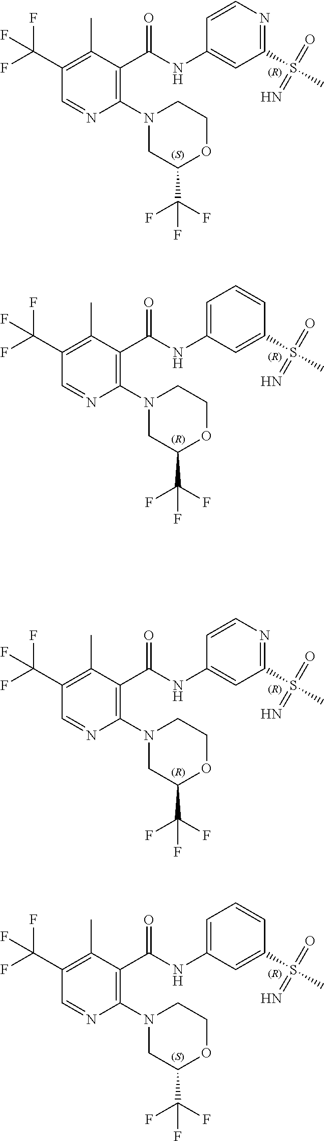

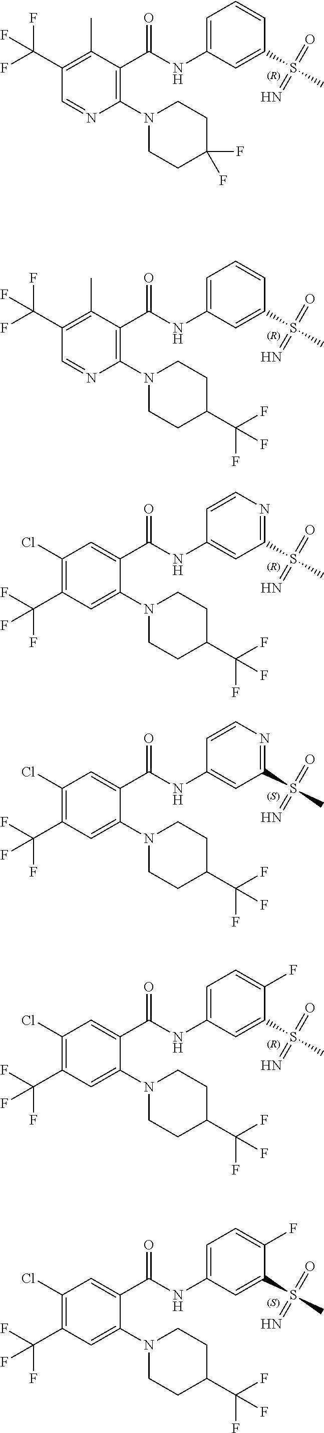

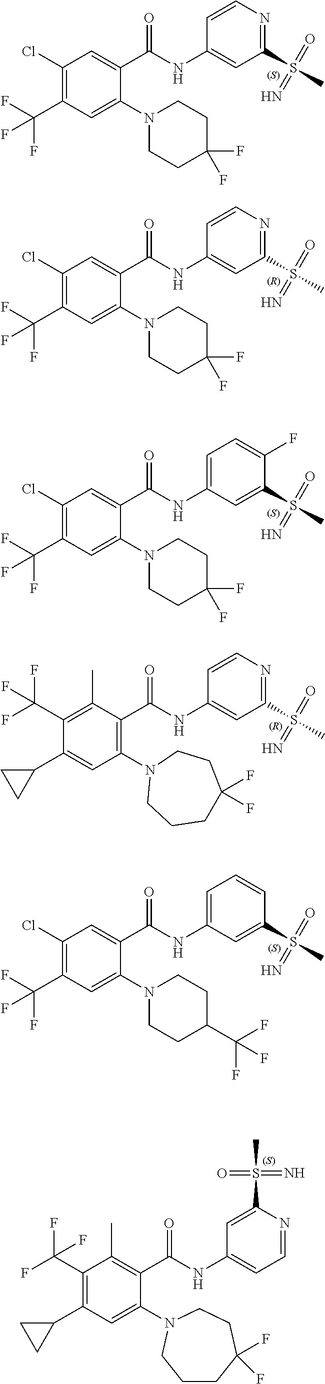

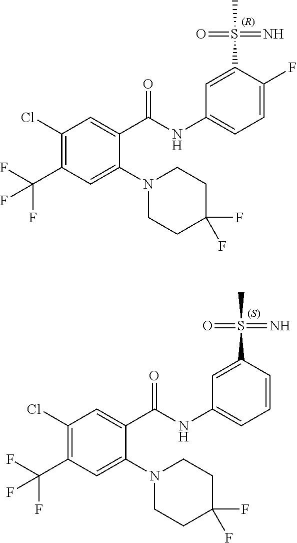

34. A method of treating a condition in a subject, the method comprising providing to a subject having a condition a compound in any of claims 1-33.

35. The method of claim 34, wherein the compound is selected from any one of the compounds recited in claims 2-34.
